(19) United States
(12) Patent Application Publication
(10) Pub. No.: US 2024/0190987 A1
(43) Pub. Date: Jun. 13, 2024

(54) Engineered EpCam Binding Antibodies

(71) Applicant: TigaTX, Boston, MA (US)

(72) Inventors: Zachary B. HILL, San Mateo, CA (US); Alexander J. MARTINKO, San Francisco, CA (US); Allison COOKE, San Francisco, CA (US); Hai TRAN, San Francisco, CA (US); Erin SIMONDS, San Francisco, CA (US); Sunandan BANERJEE, Newton Centre, MA (US)

(21) Appl. No.: 18/349,562

(22) Filed: Jul. 10, 2023

Related U.S. Application Data
(63) Continuation of application No. PCT/US2022/012056, filed on Jan. 11, 2022.
(60) Provisional application No. 63/136,111, filed on Jan. 11, 2021.

(51) Int. Cl. C07K 16/30; A61P 35/00; C07K 16/28; C07K 16/32

(52) U.S. Cl. CPC C07K 16/30; A61P 35/00; C07K 16/2803; C07K 16/32; C07K 2317/24; C07K 2317/31; C07K 2317/622

(57) ABSTRACT

The present invention uses an indinavir based mechanism of “chemically induced dimerization” to enable a precise temporal control of the activity of a T cell engaging complex in a patient.

### CROSS REFERENCE TO RELATED APPLICATIONS

This application is a continuation of International Application No. PCT/US2022/12056, filed on Jan. 11, 2022, which claims priority to U.S. Provisional Application No. 63/136,111, filed on Jan. 11, 2021, all of which are herein incorporated by reference in their entirety.

### SEQUENCE LISTING

The instant application contains a Sequence Listing which has been submitted electronically in ST.26 format and is hereby incorporated by reference in its entirety. Said copy, created on Nov. 6, 2023, is named 199828-713301 SL.xml and is 5,283,660 bytes in size.

### I. BACKGROUND OF THE INVENTION

T cell engagers are antibody derived therapeutics that transiently tether T cells via the T cell receptor complex (TCR) to surface antigens on tumor cells. This may lead to activation of T cells and direction of T cell induced lysis of the attached target tumor cells. The therapeutic potential of a T cell engager was demonstrated for example by blinatumomab, a CD19/CD3-bispecific T cell engager approved for the treatment of adult patients with relapsed/refractory acute lymphoblastic leukemia.

One of the shortcomings of the first generation of T cell engagers was a very short serum half-life. To address this, a second generation of T cell engagers was developed in which the T cell engager was fused to a human serum albumin (HSA) or Fc domain (Merlot et al., Future Med Chem. 2015; 7:553-556; Kontermann et al., Chem Biotechnol. Pharm Biotechnol. 2011; 22:868-876). However, increased serum stability has been accompanied by increased toxicity, including acute cytokine release syndrome, neurotoxicities, and/or toxicities due to engagement of the target antigen in other tissues. In some instances, such toxicities have prevented therapeutic dosing of the drugs to patients, limiting their efficacy. The toxicities are of particular concern for half-life extended T-cell engagers that may exist in a patient for weeks.

The present invention meets the need of developing more advanced therapies by providing a system that enables precise temporal control of the association of T cells with target cells, and in doing so enabling safer and more efficacious dosing of the biologics to patients.

### II. BRIEF SUMMARY OF THE INVENTION

In one aspect, the present disclosure relates to a composition comprising an indinavir binding domain comprising a) a variable heavy (VH) domain comprising a vhCDR1, vhCDR2, and vhCDR3 sequence; and b) an optional variable light (VL) domain comprising a vlCDR1, vlCDR2, and vlCDR3 sequence.

In one aspect, the present disclosure relates to a composition comprising an indinavir-complex binding domain comprising a) a variable heavy (VH) domain comprising a vhCDR1, vhCDR2, and vhCDR3 sequence; and b) a variable light (VL) domain comprising a vlCDR1, vlCDR2, and vlCDR3 sequence.

In one aspect, the present disclosure relates to a composition comprising a) a first protein comprising the composition comprising the indinavir binding domain described herein, and b) a second protein comprising the composition comprising the indinavir-complex binding domain described herein.

In one aspect, the present disclosure relates to a composition comprising a CD3 binding domain comprising a) a variable heavy (VH) domain comprising a vhCDR1, vhCDR2, and vhCDR3 sequence; and b) a variable light (VL) domain comprising a vlCDR1, vlCDR2, and vlCDR3 sequence.

In one aspect, the present disclosure related to a composition comprising a CC heterodimeric binding protein comprising: i) a first CC fusion protein comprising: 1) a first indinavir chemically induced dimerization (iCID) domain; 2) an optional domain linker, and 3) a first heterodimerization Fc domain; and ii) a second CC fusion protein comprising: 1) an anti-CD3 antigen binding domain (ABD; αCD3-ABD); 2) an optional domain linker; and 3) a second heterodimerization Fc domain. In another aspect, the present disclosure related to a composition comprising a monomeric CC binding protein comprising: a) a first indinavir chemically induced dimerization (iCID) domain; b) an optional domain linker; c) an IgG4 monomeric Fc domain; d) an optional domain linker; and e) an anti-CD3 antigen binding domain (ABD; αCD3-ABD). In another aspect, a composition comprising a CC heterodimeric binding protein comprising: a) a first CC fusion protein comprising: 1) a first indinavir chemically induced dimerization (iCID) domain; 2) an optional domain linker; 3) an αCD3-ABD; and 4) a first heterodimerization Fc domain; and ii) a second CC fusion protein comprising: a second heterodimerization Fc domain.

In one aspect, the present disclosure relates to a composition comprising a EpCAM binding domain comprising a) a variable heavy (VH) domain comprising a vhCDR1, vhCDR2, and vhCDR3 sequence; and b) a variable light (VL) domain comprising a vlCDR1, vlCDR2, and vlCDR3 sequence.

In one aspect, the present disclosure relates to a composition comprising an CT heterodimeric binding protein comprising: a) a first CT fusion protein comprising: 1) a second iCID domain; 2) an optional domain linker, and 3) a first heterodimerization Fc domain; and b) a second CT fusion protein comprising: 1) a first anti-tumor targeting ABD (αTTABD); 2) an optional domain linker, and 3) a second heterodimerization Fc domain. In another aspect, the present disclosure relates to a composition comprising a monomeric CT binding polypeptide comprising: a) a second indinavir chemically induced dimerization (iCID) domain; b) an optional domain linker(s); c) an IgG4 monomeric Fc domain; and d) an anti-tumor targeting ABD (αTTABD).

In one aspect, the present disclosure relates to a T-cell ligand induced transient engager (T-LITE) composition comprising: a) the CC binding protein described herein, and b) the CT binding protein described herein, wherein in the presence of indinavir said first and second iCID domains form a complex of said first iCID domain-indinavir-said second iCID domain, such that said T-LITE composition will bind both CD3 and said tumor.

In one aspect, the present disclosure relates to a composition comprising a CTCoS heterodimeric binding protein comprising: a) a first CTCoS fusion protein comprising: i) a second iCID domain; ii) optional domain linker(s); and iii) a first heterodimerization Fc domain; and b) a second CTCoS fusion protein comprising: i) an anti-tumor targeting antigen binding domain (αTTABD); ii) optional domain linker(s); and iii) a second heterodimerization Fc domain; wherein one of said first and second CTCoS fusion proteins further comprises a co-stimulatory domain.

In one aspect, the present disclosure relates to a co-stimulatory T-cell ligand induced transient engager (BrighT-LITE) composition comprising: a) the CC binding protein comprising the first iCID domain described herein; and b) the CTCoS binding protein comprising the second iCID domain described herein; wherein one of said first iCID domain and said second iCID domain comprises an indinavir binding domain and the other comprises an indinavir-complex binding domain, wherein in the presence of indinavir said first and second iCID domains form a complex of said first iCID domain-indinavir-said second iCID domain, such that said T-LITE composition will bind both CD3 and said tumor.

In one aspect, the present disclosure relates to a composition comprising a CTTCoS heterodimeric binding protein comprising: a) a first CTTCoS fusion protein comprising: i) a second iCID domain; ii) optional domain linker(s); iii) a first anti-tumor targeting antigen binding domain (αTTABD); iv) a first heterodimerization Fc domain; and b) a second CTTCoS fusion protein comprising: i) a T-cell co-stimulatory receptor binding domain (CoS); ii) optional domain linker(s); iii) a second αTTABD; and iv) a second heterodimerization Fc domain.

In one aspect, the present disclosure relates to a co-stimulatory dual targeting T-cell ligand induced transient engager (dual BrighT-LITE) composition comprising: a) the CC binding protein comprising the first iCID domain described herein; and b) a CTTCoS binding protein comprising the second iCID domain described herein; wherein one of said first iCID domain and said second iCID domain comprises an indinavir binding domain and the other comprises an indinavir-complex binding domain, wherein in the presence of indinavir said first and second iCID domains form a complex of said first iCID domain-indinavir-said second iCID domain, such that said T-LITE composition will bind both CD3 and said tumor.

In one aspect, the present disclosure relates to a method of treating cancer in a subject, comprising administering the composition described herein. In one aspect, the present disclosure relates to a kit comprising the composition described herein. In one aspect, the present disclosure relates to use of the composition described herein for treating cancer.

### III. BRIEF DESCRIPTION OF THE DRAWINGS

FIG. 1A illustrate a general mechanism of action of some embodiments of the invention (e.g. those that use Fc heterodimers). FIGS. 1A-1D illustrate a general T-LITE™ (“Format 1”), FIG. 2 illustrates a general brighT-LITE™ (“Format 2”), and FIGS. 3A and 3B illustrate a general “dual targeting brighT-LITE™ (“Format 3”) mechanisms of action of some embodiments of the invention (e.g. those that use Fc heterodimers). The small molecule brings together two “chemically induced dimerization” or “CID” domains and thus brings together the anti-CD3 (αCD3) antigen binding domain (ABD) and the anti-tumor target antigen (αTTA) binding domain (αTTABD), forming a final active complex and allowing for T cell engagement and tumor killing. FIGS. 1B and 1C depict exemplary final active complexes each comprising three different components: a “CT heterodimer” that has a CID domain and binds to the Tumor target antigen (and is thus a “CT” construct); a CID small molecule, that drives the formation of the complex, and a “CC heterodimer” that has a CID domain and binds to CD3, such that in the presence of the small molecule, the complex is formed and has T cell engaging activity. The Fc domains are shown as heterodimers as well. FIG. 1D depicts an example where the CC heterodimer has two tandem CID domains on one polypeptide.

FIG. 2 depicts an exemplary final active complex comprising three different components: a “CC heterodimer” that has a CID domain and a CD3 antigen binding domain (thus referred to herein as a “CC” binding protein); a “CTCoS heterodimer” that has a CID domain, a Targeting domain binding to a tumor target antigen and a Co-Stimulatory domain (CoS) (thus referred to herein as a “CTCoS” binding protein); and a CID small molecule that drives the formation of the complex.

FIG. 3 depicts an exemplary final active complex comprising three different components: a “CC heterodimer”; a “CTT heterodimer” that has a CID domain, two Targeting domains binding to two tumor targeting antigens (αTTABD) and a Co-Stimulatory domain (CoS) (thus referred to herein as a “CTTCoS” binding protein); and a CID small molecule that drives the formation of the complex. In the presence of the small molecule, the complex is formed and has T cell engaging activity. In the presence of the small molecule, the complex is formed and has T cell engaging activity. Those of skill in the art will recognize that the “αTTABD”, “αCD3,” “αCD3-ABD,” “αCD28/4-1BB,” “CoS” and “CID” domains, depicted graphically as shapes in Figures, can take on a number of different forms, including a Fab, an scFv or an scFab as disclosed herein and as known in the art.

FIG. 4 illustrates the structure of indinavir.

FIGS. 5A-5E show sequences for exemplary LS2A iCID domains that bind to indinavir. FIG. 5F shows sequences for exemplary LS2B antigens that bind to LS2A iCID domains.

FIGS. 6A-6G show sequences for exemplary LS2B iCID domains that bind to indinavir-complex. FIG. 6H shows sequences for exemplary LS2A antigens that bind to LS2B iCID domains.

FIGS. 7A-7C and 7E show exemplary sequences for αCD3 ABDs. FIG. 7D depicts exemplary CD3 antigen sequences used in Examples. In FIG. 7E, CDR sequences from the VH and VL are underlined, the linker sequences are shown in italics, and, if applicable, the junctions between domains (e.g. between the VH and VL domains and the scFv linkers in scFv formats or between the VH and CH1 or VL and CL in full length formats) are shown with slashes (“/”). As will be appreciated by those in the art and outlined herein, when scFv domains are used to bind to CD3, they can be in either orientation, VH-scFv linker-VL or VL-scFv liner-VH. As will be appreciated by those in the art, any of the variable heavy and variable light domains of the full length heavy and light chains can be used to form scFvs with an scFv linker.

FIGS. 8A-8C show exemplary sequences for αTTABDs.

FIGS. 9A-9F show exemplary sequences for CC, CT and conventional T-Cell engaging antibodies (TCE) heterodimers. The conventional TCE do not have the indinavir binding domain or the indinavir-complex binding domain.

FIG. 10 shows amino acid sequences of exemplary IgG Fc variants that find use in the present invention.

FIG. 11 shows the amino acid sequences of exemplary co-stimulatory domains. As outlined herein, the VH and VL domains can be used in different formats, including Fab, scFab and scFv constructs.

FIGS. 12A-12G illustrate different formats of CC heterodimeric binding proteins, all of which contains a first Fc fusion comprising a CID domain and a first heterodimerization Fc domain, and a second Fc fusion protein comprising an αCD3-ABD and a second heterodimerization Fc domain. FIG. 12A shows exemplary formats wherein the CID domain (e.g., BCL-2, although BCL-2 can be replaced by other CID domains, including, but not limited to, cereblon LBD, CIAP and iCID domains, with many embodiments utilizing iCID domains) is linked to the first heterodimerization Fc domain optionally via a domain linker, and the αCD3-ABD is in an scFv format and linked to the second heterodimerization Fc domain optionally via a domain linker. That is, in FIG. 12, BCL-2 can be replaced with an iCID domain.

FIG. 12B shows another exemplary format wherein the CID domain (e.g., AZ21, methotrexate ABD, both of which can be replaced by an iCID) in the format of an Fab is linked to the first heterodimerization Fc domain optionally via a domain linker, and the αCD3-ABD in an scFv format is linked to a second heterodimerization Fc domain optionally via a linker domain. FIG. 12C shows a third exemplary format wherein the CID is in the format of single chain Fab (e.g., AZ21, which can be replaced by an iCID) and linked to the first heterodimerization Fc domain optionally via a domain linker, and the αCD3-ABD is in an scFv format and linked to the second heterodimerization Fc domain optionally via a domain linker.

FIG. 12D shows further exemplary formats wherein the CID domain (again, as above, the Figures utilize BCL-2 but this domain can be replaced by an iCID domain is linked to the first heterodimerization Fc domain optionally via a domain linker on the C terminus and the αCD3-ABD in an scFv format is linked to the second heterodimerization Fc domain optionally via a linker domain on the N terminus; or the CID again, an iCID domain in many embodiments) is linked to the first heterodimerization Fc domain optionally via a domain linker on the N terminus and the αCD3-ABD in an scFv format is linked to the second heterodimerization Fc domain optionally via a linker domain on the C terminus. In all the formats, 6×His and/or FLAG epitope tags can be included in the Fc domain to facilitate purification. FIG. 12E illustrates exemplary formats of a CC heterodimeric binding protein containing an Fc fusion comprising a CID domain (e.g., an iCID domain), an αCD3-ABD and a first heterodimerization Fc domain, and an empty heterodimerization Fc domain. From the N to C terminus, the Fc fusion protein can be in the format of CID-optional domain linker-αCD3-ABD-optional domain linker-Fc, or αCD3-ABD-optional domain linker-CID-optional domain linker-Fc. The CID domain and αCD3-ABD can take various formats, for example, an scFv format. As above, Bcl-2 as labeled in these figures can be interchanged with any iCID domain as disclosed herein. FIG. 12E shows exemplary formats wherein the CID domain (e.g., iCID domains, LSA or LSB) is linked to the first heterodimerization Fc domain optionally via a domain linker, and the αCD3-ABD is in an scFv format and linked to the second heterodimerization Fc domain optionally via a domain linker. That is, in FIG. 12E, LS2A and LS2B are iCID domain. FIG. 12F shows exemplary formats wherein the CID domain (e.g., iCID domains, LSA or LSB) is linked to a second CID domain (e.g., iCID domains, LSA or LSB) via a domain linker which is then linked to the first heterodimerization Fc domain optionally via a domain linker, and the αCD3-ABD is in an scFv format and linked to the second heterodimerization Fc domain optionally via a domain linker. That is, in FIG. 12F, LS2A and LS2B are iCID domain.

FIGS. 13A-13C illustrate exemplary formats of CC heterodimeric binding proteins, each of which is composed of a first CC fusion polypeptide and second CC fusion polypeptide.

FIG. 14A-C illustrates exemplary formats of CT heterodimeric binding proteins, each of which is composed of a first CT fusion polypeptide and second CT fusion polypeptide. The CID domain, such as AZ21 and BCL-2, can be replaced by other CID domains described herein (e.g., iCID domains).

FIG. 14B depicts exemplary iCIDs of CT proteins, such as LS2A and LS2B, with monovalent engagement of EpCAM. FIG. 14C depicts exemplary iCIDs of CT proteins with bivalent engagement of EpCAM. FIGS. 14D and 14E depict additional exemplary formats a CT heterodimeric binding protein composed of a first CT fusion polypeptide and second CT fusion polypeptide.

FIG. 15 depicts the SEC-HPLC retention profiles and binding profiles to LS2A (with and without 10 μM Indinavir) measured by biolayer interferometry (BLI) for select indinavir-complex binding domains (LSBs). For SEC-HPLC data, 1.5-10 μL of 1-5 μM sample was injected onto a MabPAC 4.6×150 mm in 1×PBS-HCl at pH 7.0. BLI was performed by interrogating binding of each indinavir-complex binding domain (LSBs) to biotinylated-Ab0223 immobilized on a streptavidin biosensor. While Ab0309, Ab0310, and Ab0311 showed selective binding to Indinavir-complex, late retention times suggested non-specific column interactions. This motivated further optimization of Ab0310 to address this property.

FIG. 16 depicts improved SEC-HPLC retention profiles in LS2B after multiple rounds of engineering. For each molecule, 1.5-10 μL of 1-5 μM sample was injected onto a MabPAC 4.6×150 mm in 1×PBS-HCl at pH 7.0. Improved variants including LS2B-R5M2 and LS2B-R5M1 showed monodisperse peaks and retention times more similar to the Herceptin control, suggesting that engineering successfully mitigated non-specific column interactions.

FIG. 17 demonstrates the improvement of binding affinity of select LS2A and LS2B pairs. Binding kinetics were measured by biolayer interferometry (BLI). Biotinylated-Ab0785 (LS2A R2M34 Fab) or biotinylated-Ab0786 (LS2A R3M1 Fab) was captured by streptavidin biosensors and binding to Ab1071 (LS2B R5M2 Fab), Ab1072 (LS2B R7M1 Fab), and Ab1073 (LS2B R7M2 Fab) was measured in the presence of 10 mM indinavir. Ab1071 (LS2B R5M2 Fab) showed weaker binding to LS2A R3M1 compared to LS2A R2M34. Engineered LS2B clones R7M1 and R7M2 demonstrate improved affinities to both LS2A clones.

FIG. 18 demonstrates the improvement in acid-stability of engineered humanized LS2A antibodies. Molecules tested include Ab0188 (murine LS2A), Ab0220 (humanized LS2A), Ab0734 (humanized LS2A R2M9), and Ab0759 (humanized LS2A R2M34). The pH of the antibodies was adjusted to 3.5 and held for 1 hour at room temperature. Acid-treated and untreated samples were analyzed by size exclusion chromatography. Ab0734 and Ab0759 demonstrated improved acid-stability.

FIG. 19 demonstrates the improvement in thermostability of engineered humanized LS2A antibodies. Molecules tested include Ab0188 (murine LS2A), Ab0220 (humanized LS2A), Ab0898 (humanized LS2A R2M34), and Ab0899 (humanized LS2A R3M1). The scFv melting temperature was determined by differential scanning fluorimetry (DSF).

FIG. 20 shows the binding kinetics of free indinavir binding to LS2A R2M34 and LS2A R3M1 measured by surface plasmon resonance (SPR). Detailed methods are described in the methods section. Briefly, Ab0759 (LS2A R2M34) and Ab0899 (LS2A R3M1) were amine coupled to a CM5 sensor chip. A 3-fold serial dilution of indinavir was injected over sensor chip and binding was measured using single-cycle kinetics. The data was double reference subtracted and fit to a 1:1 Langmuir kinetic model.

FIG. 21 demonstrates LITE Switch reversibility upon indinivir (IDV) washout. Binding was measured by biolayer interferometry (BLI). Biotinylated-Ag0067 (LS2A R2M9) was captured on streptavidin biosensors. Binding of 50 nM Ab1073 (LS2B R7M2) to Ag0067 was allowed to reach steady-state in the presence of 1 μM IDV. Complex dissociation was subsequently performed for 2 hours in buffer containing (1) 50 nM Ab1073 and 1 μM IDV, (2) 50 nM Ab1073 and vehicle (washout), and (3) vehicle only. Data show that when concentrations of Ab1073 and IDV remained constant, no LITE Switch reversibility was observed. In 2 hours of IDV washout, the LITE Switch dissociated to nearly 0% complex, whereas complete complex dissociation was observed within 10 min upon removal of both Ab1073 and IDV.

FIG. 22 demonstrates that the LITE Switch assembles as a heterodimer in the presence of Indinavir. SEC complex assembly with Indinavir. Briefly, Ab0220, Ab0445, or Ab0220+Ab0445 was injected onto a MabPAC 4.6×150 mm in 1×PBS-HCl+/−10 μM Indinavir at pH 7.0. When injected individually, Ab0220 and Ab0445 each show retention times consistent with a monomer. When injected together in the presence of Indinavir, Ab0220+Ab0445 elute as a complex consistent with the molecular weight of a heterodimer.

FIG. 23 demonstrates the indinavir-dependent binding of LS2A and LS2B. Binding was measured by biolayer interferometry (BLI). Ab0902 (LS2B R5M2) was captured by anti-hIgG Fc capture (AHC) biosensors and kinetics was measured to Ab0223 (LS2A humanized parent) in the presence of 10 mM indinavir. In the absence of indinavir, no binding was detected up to a concentration of 1 mM Ab0223.

FIG. 24 depicts four LS2A variants (R3M1, R4M7, R4M8, and R4M17) that were expressed as anti-EpCAM T-LITE antibodies and assessed in a co-culture assay measuring TDCC of target cells and activation of T-cells (CD69 upregulation). The T-LITE induced activation of T-cells or killing of target cells only in the presence of indinavir, demonstrating switchable activation of the T-LITE. The degree of T-LITE activity was dose-dependent, indicating the minimum amount of anti-EpCAM T-LITE required for T-cell redirection under these conditions. T-LITE required for Some LS2A variants were more potent, exhibiting lower half-maximal effective concentration (EC50) and higher maximum Specific Cytotoxicity values. The assay was performed with 10 nM antibodies (or titrated as indicated), 1 μM indinavir or DMSO vehicle, human T-cells and HCT-116 target cells for 66 hours. In some wells, a conventional TCE (Ab1092) was titrated as a positive control.

FIG. 25 depicts four LS2A variants (R4M20, R4M23, R4M27 and R4M32) that were expressed as anti-EpCAM T-LITE antibodies and assessed in a co-culture assay as described for FIG. 24. Some LS2A variants were more potent, exhibiting lower EC50 and higher maximum Specific Cytotoxicity values.

FIG. 26 depicts four LS2A variants (R3M1, R4M7, R4M8, and R4M17) that were expressed as anti-EpCAM T-LITE antibodies and assessed in a co-culture assay measuring TDCC of target cells and activation of T-cells (CD69 upregulation). The degree of T-LITE activity was dependent on the concentration of indinavir, indicating the minimum amount of indinavir required for T-cell redirection under these conditions. Some LS2A variants were more potent, exhibiting lower EC50 and higher maximum Specific Cytotoxicity values. The assay was performed with 10 nM antibodies, indinavir (titrated as indicated) or DMSO vehicle, human T-cells and HCT-116 target cells for 66 hours.

FIG. 27 four LS2A variants (R4M20, R4M23, R4M27 and R4M32) that were expressed as anti-EpCAM T-LITE antibodies and assessed in a co-culture assay as described for FIG. 26. Some LS2A variants were more potent, exhibiting lower EC50 and higher maximum Specific Cytotoxicity values.

FIG. 28 depicts the protein expression levels of exemplary SP34 molecules. Briefly, clarified supernatants were quantified by BLI using AHQ biosensors and standard manufactures protocols. Sample concentrations were extrapolated from a standard curve generated with an IgG reference molecule diluted in Expi293F media.

FIG. 29 demonstrates the binding of humanized SP34 variants to CD3e in a single-point kinetic screen. Binding was measured by biolayer interferometry (BLI). Biotinylated-CD3e was captured by streptavidin biosensors and single-point kinetics was measured to humanized SP34 variants Ab0486-Ab0558. Figure shows the nine humanized variants that demonstrated binding to CD3e and the control Ab0599 (murine SP34).

FIG. 30 depicts eight exemplary humanized SP34 variants and the murine parental SP34 that were expressed as anti-EpCAM conventional TCE antibodies and assessed in a co-culture assay measuring TDCC of target cells and activation of T-cells (CD69 upregulation). All SP34 variants caused dose-dependent T-cell activation and target cell killing. Some SP34 variants were more potent, exhibiting lower EC50 values. The assay was performed with up to 10 nM antibodies (titrated as indicated), human T-cells and MCF-7 target cells for 70 hours.

FIG. 31 shows the pharmacokinetic profile of hSP34v68 scFv-Fc in WT mice. Ab0632 (Trastuzumab scFv-Fc) and Ab0640 (hSP34v68 scFv-Fc) were administered as a single IV dose at 6.7 mg/kg in female C57BL/6 mice aged 10-12 weeks (n=5 per group). Plasma samples were collected at 30 min, 24 hr, 72 hr, and 168 hr and concentrations of the antibodies were determined by ELISA.

FIG. 32 depicts Ab1070 binding to human and cynomolgus macaque PBMCs. Binding was measured by flow cytometry using a secondary fluorescent antibody (IgG-PE). T-cell subsets were identified using additional fluorescent antibodies against TCRab, CD4 and CD8 and gated prior to quantifying the median fluorescence intensity (MFI) of the IgG-PE channel. Binding was dose-dependent and occurred at similar EC50s across the two species, demonstrating similar binding to both species. The assay was performed with up to 10 μM antibodies (titrated as indicated).

FIG. 33 depicts anti-EpCAM Fabs expressed as conventional TCE antibodies and assessed in a co-culture assay measuring TDCC of target cells. A panel of four target cell lines was used to demonstrate that expression of human or cynomolgous macaque EpCAM on target cells was required for cell killing with antibody clone M37. The potency of killing was similar on CHO target cells recombinantly expressing human EpCAM (CHO-huEpCAM) or cynomolgus macaque EpCAM (CHO-cyEpCAM), demonstrating similar potency against target cells from the two species. A conventional TCE bearing the MOC31 anti-EpCAM Fab was included as a positive control (Ab619). Killing of HCT-116 cells was not tested with Ab682 in this experiment. The assay was performed with up to 10 nM antibodies (titrated as indicated) with human T-cells and various target cells (as indicated) for 68 hours.

FIG. 34 depicts Ab1070 binding to either an A-431 cell line that was genetically knocked-out for human EpCAM (AEKO), or AEKO cells subsequently overexpressing human EpCAM (AEPO). Binding was measured by flow cytometry using a secondary fluorescent antibody (IgG-PE) and quantified as the median fluorescence intensity (MFI) of the IgG-PE channel. Binding was dose-dependent and only observed on the AEPO cell line, demonstrating that expression of EpCAM is required for binding by the M37 anti-EpCAM clone. The assay was performed with up to 1 μM antibodies (titrated as indicated).

FIG. 35 depicts antibody clone M37 expressed as a conventional TCE antibody and assessed in a co-culture assay measuring TDCC of target cells and activation of T-cells (CD69 upregulation). Target cells were either HCT-116 cells, or an A-431 cell line that was genetically knocked-out for human EpCAM (AEKO), or AEKO cells subsequently overexpressing human EpCAM (AEPO). The assay was performed with up to 1 nM antibodies (titrated as indicated) with human T-cells and various target cells (as indicated) for 69 hours. T-cell activation and killing of target cells was only observed with AEPO or HCT-116 target cells, demonstrating a requirement for EpCAM expression for T-cell redirection by the M37 antibody clone. In a separate experiment, surface staining of EpCAM on the three cell lines was performed with a commercially available FITC-conjugated anti-EpCAM antibody (BioLegend; clone 9C4) per the manufacturer's instructions. Surface staining demonstrated that AEPO and HCT-116 cells express high levels of EpCAM whereas AEKO cells express a background level of autofluorescence.

FIG. 36 depicts schematics of formats of CCs and CTs explored in exemplary T-LITE SAR campaign. Format (Fab vs. scFv) as well as valency (1+1 vs. 2+1, vs. 2+1) were explored.

FIG. 37 depicts TDCC data for an exemplary pair from the SAR campaign, Ab439+Ab649. The T-LITE pair was assessed in a co-culture assay measuring TDCC of target cells. The T-LITE pair exhibited indinavir-dependent killing of target cells. The degree of killing was dependent on the concentration of anti-EpCAM T-LITE antibody (Ab439) and the concentration of indinavir. The assay was performed with 10 nM antibodies (or titrated as indicated), 40 μM indinavir or DMSO vehicle, human T-cells and MCF-7 target cells for 81 hours. In some wells, a conventional TCE (Ab619) was titrated as a positive control.

FIG. 38 depicts co-culture TDCC data for two exemplary T-LITE pairs. The T-LITE pairs (Ab1093+Ab1060 and Ab1094+Ab1091) were assessed in a co-culture assay measuring TDCC of target cells. Both T-LITE pairs exhibited indinavir-dependent killing of target cells. The degree of killing was dependent on the concentration of anti-EpCAM T-LITE antibody (Ab1093 or Ab1094), the concentration of anti-CD3 T-LITE antibody (Ab1060 or Ab1091), and the concentration of indinavir. The assay was performed with 10 nM antibodies (or titrated as indicated), 1 μM indinavir (or titrated as indicated) or DMSO vehicle, human T-cells and HCT-116 target cells for 69 hours.

FIG. 39 depicts co-culture TDCC data for two exemplary T-LITE pairs that exhibited indinavir-independent activity. The T-LITE pairs (Ab1059+Ab1060 and Ab1089+Ab1091) were assessed in a co-culture assay measuring TDCC of target cells. Both T-LITE pairs exhibited undesirable killing of target cells in the absence of indinavir, which could be described as lack of switchable activity. The degree of killing was dependent on the concentration of anti-EpCAM T-LITE antibody (Ab1093 or Ab1094) or the concentration of anti-CD3 T-LITE antibody (Ab1060 or Ab1091), but was independent of the concentration of indinavir. The assay was performed with 10 nM antibodies (or titrated as indicated), 1 μM indinavir (or titrated as indicated) or DMSO vehicle, human T-cells and HCT-116 target cells for 69 hours.

FIG. 40 depicts cytokine production induced by T-LITE antibodies or conventional TCE antibodies in a co-culture assay. The co-culture assay was performed with three T-LITE pairs (Ab1093+Ab1060, Ab1094+Ab1060, or Ab1094+Ab1091) as described for FIG. 38. The anti-CD3 T-LITE antibodies were titrated (as indicated) while holding the anti-EpCAM T-LITE antibodies constant at 10 nM, with or without 1 μM indinavir. In some wells, conventional TCE antibodies (Ab1070 or Ab1092) were titrated as positive controls. Cell culture media was taken for multiplexed cytokine analysis at the conclusion of the experiment (69 hours of co-culture). T-LITEs caused dose-dependent and indinavir-dependent production of cytokines, but the maximum amount of cytokine production was markedly lower than that induced by the conventional TCE controls. Even at doses of T-LITE that were sufficient to induce cytotoxicity of target cells (FIG. 38), the degree of cytokine production was not as great that induced by the conventional TCE antibodies, thereby demonstrating a decoupling of cytotoxic activity and cytokine production.

FIG. 41 depicts cytokine production by T-LITE antibodies or conventional TCE antibodies in a co-culture assay. The co-culture assay was performed with three T-LITE pairs (Ab1093+Ab1060, Ab1094+Ab1060, or Ab1094+Ab1091) as described for FIG. 38. The anti-CD3 and anti-EpCAM T-LITE antibodies were held constant at 10 nM while titrating indinavir (as indicated). Cell culture media was taken for multiplexed cytokine analysis at the conclusion of the experiment (69 hours of co-culture). T-LITEs caused dose-dependent and indinavir-dependent production of cytokines.

FIG. 42 depicts real-time measurement of TDCC in a co-culture assay for three exemplary T-LITE pairs. The T-LITE pairs (Ab778+Ab904, Ab1069+Ab1064, or Ab1069+Ab904) were assessed in a co-culture assay measuring TDCC of fluorescent-tagged target cells by real-time microscopy. T-LITE antibodies were held constant at 10 nM with indinavir (250 nM) added to some wells, as indicated (“ON”). In some wells, a conventional TCE (Ab1007) was included as a positive control. In some wells, an anti-indinavir quencher antibody (5 μM) was added at various time points, as indicated (“OFF”). All T-LITE pairs exhibited indinavir-dependent killing of target cells. In some wells, no indinavir was added (“Always OFF”). Killing was interrupted by the addition of the quencher at earlier time points (“OFF at” 30 min, 24 h, 44 h, or 49 h). Killing occurred at levels similar to the no-quencher condition (“Always ON”) if the quencher was added at the latest time point (68 h). The assay was performed with human T-cells and HCT-116-NR target cells for 120 hours. The use of a quencher to sequester indinavir in this closed in vitro system demonstrates that the formation of the active T-LITE complex can be reversed by making indinavir unavailable for complex formation, and that this reversal has functional consequences on the behavior of T-cells.

FIG. 43 depicts example expression and purification data for exemplary antibodies. SEC-HPLC data depicts a monodisperse peak for Ab1059 and Ab0160 subsequent to CH1 affinity purification and preperative SEC purification. % Main peak and % aggregate peak are denoted in each case along with final yield from 1 Liter expression in Expi293F cells. SDS-PAGE shows bands at the expected molecular weights and demonstrates that each antibody is >95% pure. Mass spectrometry using a Waters ACQUITY UPLC coupled to XevoG2-XS QTOF mass spectrometer shows peaks corresponding to the expected molecular weight (within expected accuracy of the instrument) and further supporting the purity of the preparations.

FIG. 44 shows the pharmacokinetic profile of T-LITE antibodies in WT mice. Ab1059, Ab1060, Ab1089, Ab1091 are T-LITE antibodies. Ab0654 is trastuzumab. Ab1070 is a T-cell engager antibody. All antibodies were dosed at the molar equivalent of 10 mg/kg IgG in female C57BL/6 mice (n=5 per group). Plasma samples were collected at 15 min, 1 hr, 6 hr, 24 hr, 72 hr, 144 hr, and 240 hr and concentrations of the antibodies were determined by ELISA.

FIG. 45 depicts the pharmacokinetic profile of a αCD3 T-LITE antibody in cynomolgus macaques. Cynomolgus macaques were injected with Ab1060 as a single intravenous infusion over 15 minutes at doses from 0.1 to 3.0 mg/kg (n=1 animals per dose level). Blood samples were collected at 1 hr, 3 hr, 6 hr, 1 d, 2 d, 3 d, 4 d, 5 d, 6 d, 7 d, 8 d, 9 d, and 10 d post-infusion with potassium EDTA as the anticoagulant, then processed to plasma and frozen. Antibody concentrations in plasma were measured using an ELISA specific for human IgG. Ab1060 exhibited a plasma half-life of 4-6 days in cynomolgus macaques.

FIG. 46 depicts dose-dependent binding of a αCD3 T-LITE antibody to the surface of peripheral T-cells cynomolgus macaques. Cynomolgus macaques were injected with Ab1060 as a single intravenous infusion over 15 minutes at doses from 0.1 to 3.0 mg/kg (n=1 animals per dose level). Whole blood samples were collected at 1, 24, 48, 72, 96, 168, 192 and 240 hours post-infusion with potassium EDTA as the anticoagulant. The amount of surface-bound Ab1060 was detected using a fluorescent PE-conjugated anti-human IgG antibody measured by flow cytometry. The median fluorescence intensity (MFI) was normalized to the maximum MFI observed across all samples in the experiment. Ab1060 exhibited dose-dependent binding to CD4+ T-cells in cynomolgus macaques.

FIG. 47 depicts changes in body weight and plasma cytokine levels in B-hCD3E transgenic mice treated with a single dose of the αCD3 T-LITE antibody or a control TCE. The control TCE contains an anti-mouse EpCAM Fab instead of the LS2B domains, and would therefore be expected to redirect T-cells to target endogenous EpCAM-expressing tissues. Mice were injected with Ab1062 or Ab1060 as a single bolus intravenous injection at doses of 1 or 10 mg/kg, respectively (n=4 animals per dose level). Body weights were measured 3 and 5 days post-injection. Whole blood samples were collected at −96, 2, 6, 24, 72, and 120 hours relative to the time of injection, with potassium EDTA as the anticoagulant. The concentration of 18 cytokines in the plasma were measured using a bead-based multiplex assay. The control TCE, Ab1062, induced body weight loss at dose of 1 mg/kg, whereas Ab1060 did not at a dose of 10 mg/kg. Furthermore, Ab1062 caused dramatic cytokine release within 24 hours of injection, whereas Ab1060 caused a much lower degree of cytokine release.

FIG. 48 depicts co-culture TDCC data for an exemplary T-LITE pair and a conventional TCE on isogenic cell lines with different surface expression of human EpCAM. A panel of isogenic cell lines was created using the Calu-6 human lung cancer cell line as a starting point. Based on literature values for surface expression of EpCAM, Calu-6 expresses 40,926 copies/cell, whereas HCT-116 expresses approximately 100-fold higher levels at 4,753,190 copies/cell (Casaletto et al., PNAS 2019; doi: 10.1073/pnas.1819085116). Human EpCAM was expressed on the Calu-6 background using lentiviral vectors with different strength promoters. Subclones were isolated, resulted in an isogenic panel of four Calu-6 EpCAM-overexpressing (CEPO) cell lines with varying levels of EpCAM expression (CEPO-L2A2, CEPO-HB3, CEPO-HA2, and CEPO-HB2). Relative surface expression of human EpCAM on each cell line was measured by flow cytometry, as well as parental Calu-6 cells and HCT-116 as controls. The conventional TCE control Ab1070 and a T-LITE pair (Ab1093+Ab1060) were assessed in a co-culture assay measuring TDCC of either CEPO-HB2 or CEPO-L2A2 target cells, representing high and low expression of EpCAM, respectively. The assay was performed with 10 nM antibodies (or titrated as indicated), 100 nM indinavir or DMSO vehicle, human T-cells and a variety target cells (as indicated) for 67 hours. Flow cytometry was performed using a PE-conjugated fluorescent anti-human EpCAM antibody (clone 9C4).

FIGS. 49 and 50 depict sequences for additional exemplary iCID domains.

### IV. DETAILED DESCRIPTION OF THE INVENTION

### A. Introduction

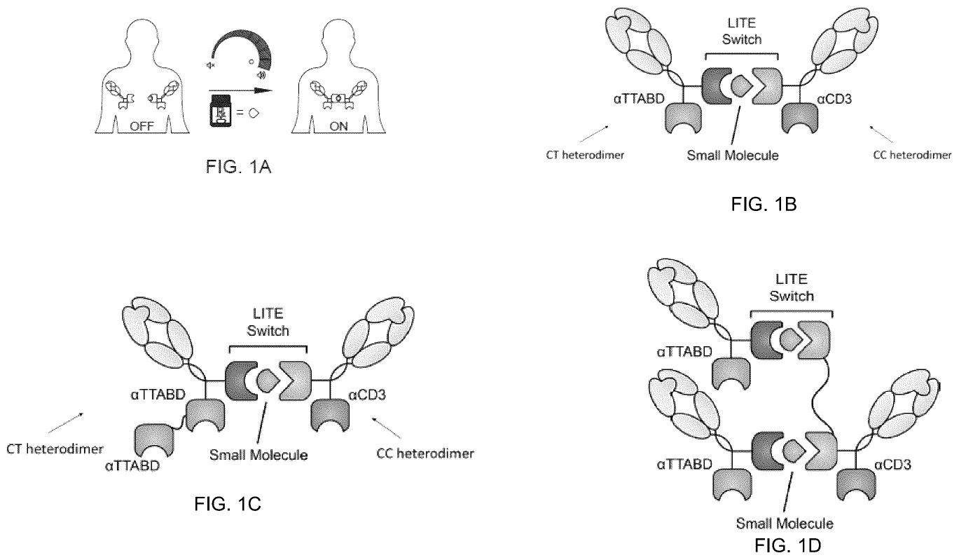

Major limitation of current T cell engaging therapeutics is their toxicity, including acute cytokine release syndrome, neuro toxicities, and/or “on-target off-tumor” toxicities (wherein the therapeutic binds to normal tissue rather than tumor tissue). The present invention may address the drawbacks of current T cell engaging therapeutics by controlling the formation of the T cell engaging complexes in such a way that both its formation and its dissociation can be controlled using small molecules as generally outlined in FIGS. 1A-1D, 2 and 3. This mechanism is generally referred to herein as “chemically induced dimerization” or “CID”. Two CID domains may not bind to each other in the absence of a third component, the small molecule. The two CID domains can be brought together by a small molecule (referred to generally herein as a “CID small molecule”, or “CID-SM”). In some embodiments, an anti-CD3 antigen binding domain (αCD3-ABD) is linked to one or more CID domains, and one or more anti-tumor targeting antigen binding domains (αTTABD) are linked to the other CID domain. Furthermore, in some embodiments, the present invention includes the use of a co-stimulatory activity by linking a T cell co-stimulatory domain directly or indirectly to the αTTABDs. In one aspect, a small molecule brings together the two CID domains, and thus brings together the αCD3-ABD, αTTABDs and co-stimulatory domain, allowing for T cell engagement and tumor killing.

However, when administration of the small molecule drug to the patient is stopped or reduced, the T cell engaging complex can then disassociate and any toxicities of the complex are decreased. That is, the use of these two CID domains thus function as a molecular “switch”, allowing control over the biological activity of the complexes. Manipulating the concentration of small molecule can enable temporal or spatial control over the formation of the functional T cell engaging complexes. Temporal control may be achieved by changing the amount of small molecule in the blood (e.g., by increasing, decreasing, or halting the administration of small molecule to the patient). In another aspect, spatial control may be achieved by injecting the small molecule at a specific site of desired drug activity (e.g., via intratumoral injection). Pulsatile dosing of the small molecule may be used to provide periods of activation, rest and re-activation to the T cells, thereby mimicking repeated exposures to a natural pathogen.

In the present case, the small molecule is indinavir. The structure of indinavir is shown in FIG. 4. Accordingly, there are two different CID domains that utilize indinavir, referred to herein as “indinavir chemically induced dimerization domains,” “iCID domains,” or “iCIDs”. One of the iCID domains binds indinavir, and is called an indinavir binding domain. The other iCID domain binds the complex of the first iCID with indinavir and is referred to herein as the “indinavir-complex binding domain”. That is, the indinavir-complex binding domain will not bind to the first iCID if indinavir is not present. In general, as outlined herein, these iCID domains are generally Fv domains, and most usually scFv domains comprising variable heavy domains and variable domains linked by a scFv linker in different orientations, as described more fully below.

There are three basic formats of the compositions of the invention as outlined herein, and all are referred to as T-cell Ligand Induced Transient Engager (“T-LITEs™”) compositions, sometimes referred to as “complexes” as shown in FIGS. 1A-1D, 2 and 3. In the first, “standard T-LITE™ format”, (“Format 1”) the compositions have two separate protein components that act in tandem, or in pairs, to give functionality when exposed to indinavir that brings the two protein components together in a complex. However, without the iCID small molecule, the T-LITE™ compositions do not have the required two functions of a T cell engager in one complex: the ability to bind CD3 (and thus activate T cell-mediated cytotoxicity) and the ability to bind a tumor cell.

As discussed more fully below, in one aspect, the iCID domains of the invention function generally in pairs. In an exemplary T-LITE™ composition, one protein component has one or more iCID domains and an αCD3-ABD (which as described more fully herein can take on a number of different forms); this protein component is referred herein as the “CC” binding protein, since it has a iCID domain and an anti-CD3 antigen binding domain. The other protein component of the T-LITE™ composition has the other iCID domain and an αTTABD, referred to generally as the “CT” binding protein, since it has a iCID and anti-Tumor targeting antigen binding domain. Additionally, in many embodiments herein, the functional domains of each protein component can be assembled using Fc domains, which spontaneously self-assemble. In many embodiments, the inventions rely on Fc domains that contain amino acid modifications that result in “heterodimerization”, wherein two non-identical Fc domains will self-assemble, thus bringing the two functionalities together into either a CT fusion protein or a CC fusion protein. The CT fusion protein and the CC fusion protein then come together in the presence of indinavir, to form an active T cell engaging complex, as generally shown in FIG. 1B.

Additionally, as is further discussed below, each protein component of the T-LITE™ complex can have a number of different formats, in terms of the order of the functional domains within the polypeptide chains. In some instances, the selected arrangement of domains within proteins employed in the T-LITE composition provides for an improvement, e.g., in synthesis, stability, affinity or effector function, over others of these structures or those known in the art.

An additional exemplary format of the present invention is called a “BrighT-LITE™” format (“Format 2”) which additionally contain one or more T cell co-stimulatory domain(s). The compositions are referred to herein as BrighT-LITEs™, because they include co-stimulatory targeting domains, that serve to “turn up” the T-LITE™s. The BrighT-LITE™ compositions, like Format 1, have two separate components that act in tandem, or in pairs, to give functionality when exposed to indinavir that brings the two components together in a complex. However, without indinavir, the BrighT-LITE™ compositions do not have the required two functions of a T cell engager in one complex: the ability to bind CD3 (and thus activate T cell-mediated cytotoxicity) and the ability to bind a tumor cell.

As discussed more fully below, and similar to Format 1, in one aspect, the iCID domains of the invention function generally in pairs. In Format 2, one protein component has one or more iCID domains and an αCD3-ABD (which as described more fully herein can take on a number of different forms); this protein component is referred herein as the “CC” binding protein, since it has an iCID domain and an anti-CD3 antigen binding domain. The other protein component of the BrighT-LITE™ composition has the other iCID domain, an αTTABD and a Co-Stimulatory domain; this protein component is referred herein as the “CTCoS” binding protein. All three formats of the invention utilize CC binding proteins.

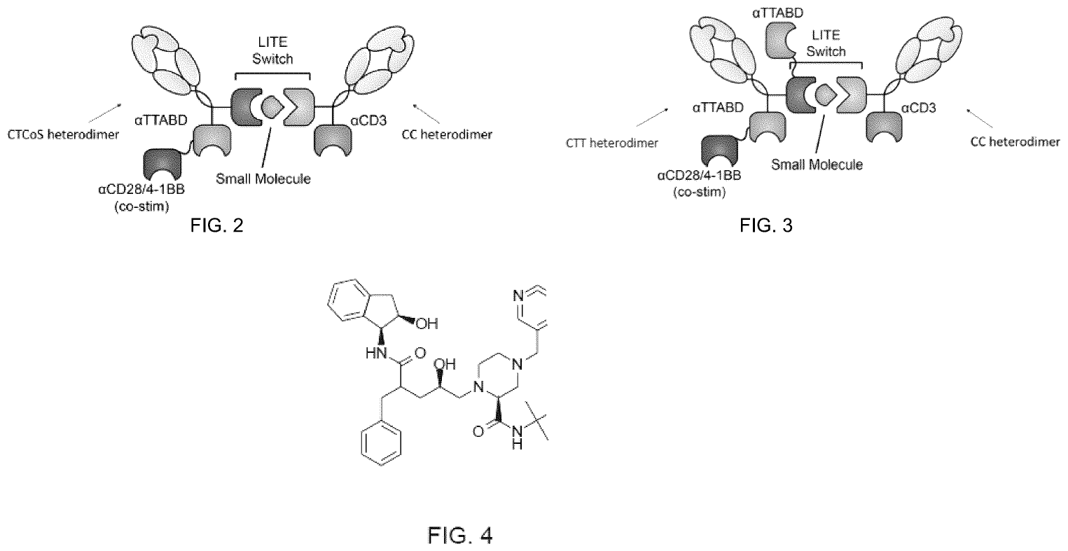

Additionally, in many embodiments herein, as is true for Format 2, the functional domains of each protein component can be assembled using Fc domains, which spontaneously self-assemble. In many embodiments, the inventions rely on Fc domains that contain amino acid modifications that result in “heterodimerization”, wherein two non-identical Fc domains will self-assemble, thus bringing the two functionalities together into either a CTCoS binding protein or a CC binding protein. The CTCoS binding protein and CC binding protein then come together in the presence of the CID small molecule, to form an active T cell engaging complex, as generally shown in FIG. 2.

Additionally, as is further discussed below, each protein component of the Format 2 complex can have a number of different formats, in terms of the order of the functional domains within the polypeptide chains as well as the number of polypeptide chains in each protein. In some instances, the selected arrangement of domains within proteins employed in a BrighT-LITE™ format composition provides for an improvement, e.g., in synthesis, stability, affinity or effector function, over the structures generally known in the art.

The third exemplary format, “Format 3”, is a dual targeting BrighT-LITEs™, because Format 3 constructs include a second targeting domain. As for Formats 1 and 2, the iCID domains of the invention function generally in pairs. In Format 3 compositions, one protein component has one iCID domain and an αCD3-ABD (which as described more fully herein can take on a number of different forms); this protein component is referred herein as the “CC” binding protein, since it has a iCID domain and an anti-CD3 antigen binding domain. The other protein component of the Format 3 composition comprises the other iCID domain, two or more αTTABDs and a Co-Stimulatory domain; this protein component is referred herein as the “CTTCoS” binding protein.

In the Format 3 compositions, engagement of the co-stimulatory domain can increase the activation state of the T-cell resulting in enhanced cytotoxicity, and enhanced cytokine profiles relative to a bispecific T-cell engager comprising an αCD3-ABD and αTTABD without a co-stimulatory domain. The two or more αTTABDs can bind to the same tumor antigen or two different tumor antigens. In one aspect, advantages of having two or more αTTABDs can include conferring increased potency to tumor targeting antigens (TTAs) due to increased avidity provided by the two tumor antigen binders. Further, in some embodiments, an αTTABD with a lower affinity can be used to increase selectivity of a CTTCoS binding protein. Use of multivalent interactions can favor association of a CTTCoS binding protein with cells expressing high levels of a TTA. Therefore, in some instances, selectivity for high-TTA expressing tumor cells can be achieved over healthy tissue expressing lower levels of the TTA.

Additionally, in many embodiments herein, the functional domains of each protein component can be assembled using Fc domains, which spontaneously self-assemble. In many embodiments, the inventions rely on Fc domains that contain amino acid modifications that result in “heterodimerization”, wherein two non-identical Fc domains will self-assemble, thus bringing the two functionalities together into either a CTTCoS binding protein or a CC binding protein. The CTTCoS binding protein and CC binding protein then come together in the presence of the iCID small molecule, to form an active T cell engaging complex, as generally shown in FIG. 3.

Additionally, as is further discussed below, each protein component of the Format 3 compositions can have a number of different formats, in terms of the order of the functional domains within the polypeptide chains as well as the number of polypeptide chains in each protein. In some instances, the selected arrangement of domains within proteins employed in a BrighT-LITE composition provides for an improvement, e.g., in synthesis, stability, affinity or effector function, over the structures generally known in the art.

### B. Definitions

In order that the application may be more completely understood, several definitions are set forth below. Such definitions are meant to encompass grammatical equivalents.

Unless explained otherwise, all technical and scientific terms used herein have the same meaning as commonly understood to one of ordinary skill in the art to which this disclosure belongs.

Accession Numbers: Reference numbers assigned to various nucleic acid and amino acid sequences in the NCBI database (National Center for Biotechnology Information) that is maintained by the National Institute of Health, U.S.A. The accession numbers listed in this specification are herein incorporated by reference as provided in the database as of the date of filing this application.

The term “antigen binding domain” or “ABD” herein is meant a set of six Complementary Determining Regions (CDRs) that, when present as part of a polypeptide sequence, specifically binds a target antigen as discussed herein. Thus, for example, an ABD that binds CD3 is referred to herein as a “αCD3-ABD”. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light chain. The CDRs are present in the variable heavy domain (VH) and variable light domain (VL), respectively, and together form an Fv region. Thus, in some cases, the six CDRs of the antigen binding domain are contributed by a variable heavy and variable light chain. For example, in a scFv format, the VH and VL domains are covalently attached, generally through the use of a linker as outlined herein, into a single polypeptide sequence, which can be either (starting from the N-terminus) VH-linker-VL or VL-linker-VH, with the former being generally preferred (including optional domain linkers on each side, depending on the format used). In some cases, the linker is a domain linker as described herein. Thus, included within the definition of “antigen binding domains” are “scFv antigen binding domains”, or scFv-ABDs.

Additionally, in some cases, an ABD used in the invention can be a single domain ABD (“sdABD”). By “single domain Fv”, “sdFv” or “sdABD” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013). These are sometimes referred to in the art as “VHH” domains.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the VHCDRs (e.g., VHCDR1, VHCDR2 and VHCDR3) and the disclosure of each variable light region is a disclosure of the VLCDRs (e.g., VLCDR1, VLCDR2 and VLCDR3).

A useful comparison of CDR numbering is as below, see Lafranc et al., Dev. Comp. Immunol. 27(1):55-77 (2003). Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., SEQUENCES OF PROTEINS OF IMMUNOLOGICAL INTEREST, 5th Ed. Public Health Service, National Institutes of Health, Bethesda, Md. (1991)).

TABLE 1

| | Kabat + | | | | | |
|---|---|---|---|---|---|---|
| | Chothia | IMGT | Kabat | AbM | Chothia | Contact |
| VHCDR1 | 26-35 | 27-38 | 31-35 | 26-35 | 26-32 | 30-35 |
| VHCDR2 | 50-65 | 56-65 | 50-65 | 50-58 | 52-56 | 47-58 |
| VHCDR3 | 95-102 | 105-117 | 95-102 | 95-102 | 95-102 | 93-101 |
| VLCDR1 | 24-34 | 27-38 | 24-34 | 24-34 | 24-34 | 30-36 |
| VLCDR2 | 50-56 | 56-65 | 50-56 | 50-56 | 50-56 | 46-55 |
| VLCDR3 | 89-97 | 105-117 | 89-97 | 89-97 | 89-97 | 89-96 |

By “domain linker” or grammatical equivalents herein is meant a linker that joins two protein domains together, such as those used in linking the different domains of a protein. As is more fully described below, generally, there are a number of suitable linkers that can be used, including traditional peptide bonds, generated by recombinant techniques that allows for recombinant attachment of the two domains with sufficient length and flexibility to allow each domain to retain its biological function.

“Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope. Epitopes may be either conformational or linear. A conformational epitope is produced by spatially juxtaposed amino acids from different segments of the linear polypeptide chain. A linear epitope is produced by adjacent amino acid residues in a polypeptide chain. Conformational and linear epitopes may be distinguished in that the binding to the former but not the latter is lost in the presence of denaturing solvents.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded by DNA, e.g., the 20 amino acids that have codons in DNA and RNA.

By “amino acid substitution” or “substitution” herein is meant the replacement of an amino acid at a particular position in a parent polypeptide sequence with a different amino acid. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 #designates a deletion of the sequence GluAspAla that begins at position 233.

By “variant”, e.g., “variant polynucleotide sequence”, “variant amino acid sequence”, “variant polypeptide”, “variant protein” or “protein variant”, as used herein is meant a composition, e.g., a polynucleotide sequence, amino acid sequence, polypeptide or protein, that differs from that of a respective parent composition, e.g., a polynucleotide, amino acid sequence, polypeptide or protein, by virtue of at least one modification, e.g., a nucleotide or an amino acid modification. For example, protein variant may refer to the protein itself, a composition comprising the protein, or the amino acid sequence that encodes it. Variant polypeptide may refer to the polypeptide itself, a composition comprising the polypeptide, or the amino acid sequence that encodes it. Variant polynucleotide may refer to the polynucleotide itself, a composition comprising the polynucleotide, or the nucleic acid sequence that encodes it. Generally, unless otherwise noted herein, the parent composition is a wild type polynucleotide, polypeptide or protein

By “wild type or WT”, e.g. “wild type nucleotide sequence”, “wild type amino acid sequence”, “wild type polypeptide, or “wild type protein”, as used herein is meant a composition, e.g., a nucleotide sequence, amino acid sequence, polypeptide, or protein, that is found in nature, including allelic variations. A WT protein has an amino acid sequence or a nucleotide sequence that has not been intentionally modified.

By non-naturally occurring, e.g., a “non-naturally occurring variant” or “non-naturally occurring modification” it is meant an amino acid modification that is not observed in nature. As one nonlimiting example, a non-naturally occurring variant IgG domain would include an IgG domain comprising an amino acid modification that is not isotypic. For example, because none of the IgGs comprise an alanine at position 234 and 235, the substitution 234A and 235A in IgG1 and IgG4 is considered a non-naturally occurring modification at position 234.

As used herein, “protein” herein is meant at least two covalently attached amino acids, which includes proteins, polypeptides, oligopeptides and peptides. A protein comprises naturally occurring amino acids and peptide bonds. In addition, a protein may include synthetic derivatization of one or more side chains or termini, glycosylation, PEGylation, circular permutation, cyclization, linkers to other molecules, fusion to proteins or protein domains, and addition of peptide tags or labels. “Polypeptide”, as a subset of “protein”, refers to a protein that is a single amino acid chain, while “protein” can refer to one or more amino acid chains.

By “residue” as used herein is meant a position in a protein and its associated amino acid identity.

By “Fab” or “Fab region” as used herein is meant the polypeptide that comprises the VH, CH1, VL, and CL immunoglobulin domains. Fab may refer to this region in isolation, or this region in the context of a full length antibody or antibody fragment. In some cases, as generally outlined below, the Fabs can be single chain Fabs (scFab), that have the VH-CH1 domains linked by a scFab linker of an appropriate length and flexibility to the VL-CL domains, wherein the scFab retains the specificity of the intact antibody from which it is derived. These domains can be in either the (N- to C-terminal) VH-CH1-scFab linker-VL-CL or VL-CL-scFab linker-VH-CH1 orientations.

By “Fv” or “Fv fragment” or “Fv region” as used herein is meant a polypeptide that comprises the VL and VH domains of a single antibody. As will be appreciated by those in the art, an Fv is made up of two domains, a variable heavy domain and a variable light domain. In the case of sdABDs, the Fv domain comprises just a VHH domain.

As used herein, “single chain variable fragment” or “scFv” refers to an antibody fragment comprising a variable heavy domain and a variable light domain, wherein the variable heavy domain and a variable light domain are contiguously linked via a short flexible polypeptide linker, and capable of being expressed as a single polypeptide chain, and wherein the scFv retains the specificity of the intact antibody from which it is derived. The variable heavy domain and a variable light domain of a scFv can be, e.g., in any of the following orientations: variable light domain-scFv linker-variable heavy domain or variable heavy domain-scFv linker-variable light domain.

By “effector function” as used herein is meant an effector function of an antibody Fc region, which is a biochemical event that results upon binding of an antibody Fc region with an Fc receptor or ligand, e.g. ADCC, ADCP, CDC, and the like.

By “Fc” or “Fc region” or “Fc domain” as used herein is meant a polypeptide comprising the constant region of an antibody, in some instances, excluding all of the first constant region immunoglobulin domain (e.g., CH1) or a portion thereof, and in some cases, optionally including all or part of the hinge domain. The Fc domain disclosed herein may be Fc domain from IgG. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminus, CH2-CH3 or hinge-CH2-CH3. In some embodiments, the Fc domain is that from IgG1, IgG2, IgG3 or IgG4, with IgG1 hinge-CH2-CH3 finding particular use in many embodiments. Additionally, in certaIn some embodiments, wherein the Fc domain is a human IgG1 Fc domain, the hinge includes a C220S amino acid substitution. Furthermore, in some embodiments where the Fc domain is a human IgG4 Fc domain, the hinge includes a S228P amino acid substitution. Although the boundaries of the Fc region may vary, the human IgG heavy chain Fc region is usually defined to include residues E216, C226, or A231 to its carboxyl-terminus, wherein the numbering is according to the EU index as in Kabat. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. In some embodiments, as is more fully described below, amino acid modifications are made to the Fc region, for example to alter binding to one or more FcγR or to the FcRn.

By “Fc gamma receptor”, “FcγR” or “FcgammaR” as used herein is meant any member of the family of proteins that bind the IgG antibody Fc region and is encoded by an FcγR gene. In human this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. In some cases, as outlined herein, binding to one or more of the FcγR receptors is reduced or ablated. In such cases, Fc effector function is reduced. For example, reducing binding to FcγRIIIa reduces ADCC, and in some cases, reducing binding to FcγRIIIa and FcγRIIb is desired.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. As discussed herein, binding to the FcRn receptor is desirable, and in some cases, Fc variants can be introduced to increase binding to the FcRn receptor.

“Fc variant” or “variant Fc” as used herein is meant a protein comprising an amino acid modification in an Fc domain. The modification can be an addition, deletion, or substitution. The Fc variants of the present invention are defined according to the amino acid modifications that compose them. Thus, for example, Fc L234A/L235A is an Fc variant with the substitution for at position relative to the parent Fc polypeptide, wherein the numbering is according to the EU index. The identity of the wildtype amino acid may be unspecified, in which case the aforementioned variant is referred to as Fc 234A/235A. It is noted that the order in which substitutions are provided is arbitrary, that is to say that, for example, is the same Fc variant as, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

By “fusion protein” or “fusion polypeptide” as used herein is meant covalent joining of at least two proteins or protein domains, to form a protein of a single amino acid chain. Fusion proteins may comprise artificial sequences, e.g. a domain linker, and a CID domain as described herein and an αCD3-ABD or an αTTABD. By “Fc fusion protein” herein is meant a protein comprising an Fc domain, generally linked (optionally through a domain linker, as described herein) to one or more different protein domains. In most instances, two Fc fusion proteins will dimerize and form a homodimeric Fc protein or a heterodimeric Fc protein. In some embodiments, a heterodimeric Fc protein includes an Fc domain alone (e.g., an “empty Fc domain”) and an Fc fusion protein. In some embodiments, a heterodimeric Fc protein includes two Fc fusion proteins. In some embodiments the Fc domain is monomeric, such as when a variant IgG4 Fc domain is used, which does not self-assemble into a dimer.

By “fused” or “covalently linked” is herein meant that the components (e.g., a CID domain and an Fc domain) are linked by peptide bonds, either directly or indirectly via domain linkers, outlined herein.

By “heavy constant region” herein is meant the CH1-hinge-CH2-CH3 portion of a IgG antibody.

By “light constant region” is meant the CL domain from kappa or lambda.

By “amino acid” as used herein is meant one of the 20 naturally occurring amino acids that are coded for by DNA and RNA.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is subsequently modified to generate a variant, and by “parent antibody” as used herein is meant an unmodified antibody that is subsequently modified to generate a variant antibody. It should be noted that “parent antibody” includes known commercial, recombinantly produced antibodies as outlined below.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. In the present case, for example, the target antigen of interest herein can be a CD3 protein or a tumor targeting antigen including a CD19 protein. Thus, an “anti-CD19 binding domain” is a target tumor antigen (TTA) binding domain where the target antigen is CD19. Additional target antigens are outlined below.

By “target cell” as used herein is meant a cell that expresses a target antigen.

By “variable domain” as used herein is meant the region of an immunoglobulin that comprises one or more Ig domains substantially encoded by any of the Vκ (V.kappa), Vλ (V.lamda), and/or VH genes that make up the kappa, lambda, and heavy chain immunoglobulin genetic loci respectively. Thus a “variable heavy domain” comprises (VH)FR1-vhCDR1-(VH)FR2-vhCDR2-(VH)FR3-vhCDR3-(VH)FR4 and a “variable light domain” comprises (VL)FR1-vlCDR1-(VL)FR2-vlCDR2-(VL)FR3-vlCDR3-(VL)FR4.

The antibodies of the present invention are generally recombinant. “Recombinant” means the antibodies are generated using recombinant nucleic acid techniques in exogenous host cells.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “K_{D}”, as used herein, is intended to refer to the dissociation constant, which is obtained from the ratio of Kd to Ka (i.e., Kd/Ka) and is expressed as a molar concentration (M). K_{D} values for antibodies can be determined using methods well established in the art. In some embodiments, the method for determining the K_{D} of an antibody is by using surface plasmon resonance, for example, by using a biosensor system such as a BIACORE® system. In some embodiments, the K_{D} of an antibody is determined by Bio-Layer Interferometry. In some embodiments, the K_{D} is measured using flow cytometry with antigen-expressing cells. In some embodiments, the K_{D} value is measured with the antigen immobilized. In other embodiments, the K_{D} value is measured with the antibody (e.g., parent mouse antibody, chimeric antibody, or humanized antibody variants) immobilized. In certaIn some embodiments, the K_{D} value is measured in a bivalent binding mode. In other embodiments, the K_{D} value is measured in a monovalent binding mode. Specific binding for a particular antigen or an epitope can be exhibited, for example, by an antibody having a K_{D} for an antigen or epitope of at least about 10⁻⁷ M, at least about 10⁻⁸ M, at least about 10⁻⁹ M, at least about 10⁻¹⁰ M, at least about 10⁻¹¹ M, at least about 10⁻¹² M, at least about 10⁻¹³ M, or at least about 10⁻¹⁴ M. Typically, an antibody that specifically binds an antigen will have a K_{D} that is 20-, 50-, 100-, 500-, 1000-, 5,000-, 10,000- or more times greater for a control molecule relative to the antigen or epitope.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

An example of an implementation of an algorithm to determine percent identity of a sequence is provided by the Genetics Computer Group (Madison, WI) in the “BestFit” utility application. The default parameters for this method are described in the Wisconsin Sequence Analysis Package Program Manual, Version 8 (1995) (available from Genetics Computer Group, Madison, WI). Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, CA). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http://in front of blast.ncbi.nlm.nih.gov/Blast.cgi.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

The terms “treatment”, “treating”, “treat”, and the like, refer to obtaining a desired pharmacologic and/or physiologic effect. The effect may be prophylactic in terms of completely or partially preventing a disease or symptom thereof or reducing the likelihood of a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, e.g., arresting its development or progression; and (c) relieving the disease, e.g., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

An “effective amount” or “therapeutically effective amount” of a composition includes that amount of the composition which is sufficient to provide a beneficial effect to the subject to which the composition is administered. An “effective amount” of a delivery vehicle includes that amount sufficient to effectively bind or deliver a composition.

The term “nucleic acid” includes RNA or DNA molecules having more than one nucleotide in any form including single-stranded, double-stranded, oligonucleotide or polynucleotide. The term “nucleotide sequence” includes the ordering of nucleotides in an oligonucleotide or polynucleotide in a single-stranded form of nucleic acid.

A “vector” is capable of transferring gene sequences to a target cell. Typically, “vector construct,” “expression vector,” and “gene transfer vector,” mean any nucleic acid construct capable of directing the expression of a gene of interest and which can transfer a gene sequence to a target cell, which can be accomplished by genomic integration of all or a portion of the vector, or transient or inheritable maintenance of the vector as an extrachromosomal element. Thus, the term includes cloning, and expression vehicles, as well as integrating vectors.

### C. T-Cell Ligand Induced Transient Engager Compositions

Accordingly, in some aspects, the present invention provides T-cell Ligand Induced Transient Engager (T-LITE™) compositions that can take on three different formats (Format 1 is depicted in FIGS. 1A-1D, Format 2 is depicted in FIG. 2, and Format 3 is generally depicted in FIG. 3. Each format relies on the use of multiple protein domains that together form fusion proteins that interact in pairs along with indinavir to form active T cell engaging complexes as more fully described below.

1. Indinavir Chemically Induced Dimerization (iCID) Domains

Chemically induced dimerization is a biological mechanism in which two proteins non-covalently associate or bind only in the presence of a dimerizing agent. In the present invention, the two proteins are referred to as indinavir Chemically Induced Dimerization (iCID) domains, and the dimerizing agent is referred to as a “Chemically Induced Dimerization small molecule” or a “CID small molecule” or “CIDSM” or “indinavir”.

In the present invention, iCID domains come in pairs that will associate in the presence of indinavir. In the present invention, the iCID pairs are made up of two different iCID domains that are brought together by indinavir: one iCID is an indinavir binding domain that binds indinavir, and the second iCID is an indinavir-complex binding domain, wherein the second iCID only binds to the first iCID when it is complexed with indinavir.

a. Indinavir Binding Domains as iCIDs

As discussed herein, one of the iCID pair is an indinavir binding domain, which are generally antigen binding domains (ABDs) that bind indinavir. In some embodiments, the indinavir binding domain is a Fab. In many embodiments, the indinavir binding domain is a scFv that binds indinavir. In some embodiments, the iCID pair comprises an indinavir binding domain comprising: a) a variable heavy (VH) domain comprising a vhCDR1, vhCDR2, and vhCDR3 sequence; b) a variable light (VL) domain comprising a vlCDR1, vlCDR2, and vlCDR3 sequence. In some embodiments, the iCID is an scFv comprising an indinavir binding domain comprising: a) a variable heavy (VH) domain comprising a vhCDR1, vhCDR2, and vhCDR3 sequence; b) a variable light (VL) domain comprising a vlCDR1, vlCDR2, and vlCDR3 sequence. In one aspect, a composition of the present disclosure comprises of an indinavir binding domain comprising: a) a variable heavy (VH) domain comprising a vhCDR1, vhCDR2, and vhCDR3 sequence; b) an optional variable light (VL) domain comprising a vlCDR1, vlCDR2, and vlCDR3 sequence. In another aspect, the composition comprises b) the variable light (VL) domain comprising a vlCDR1, vlCDR2, and vlCDR3 sequence.

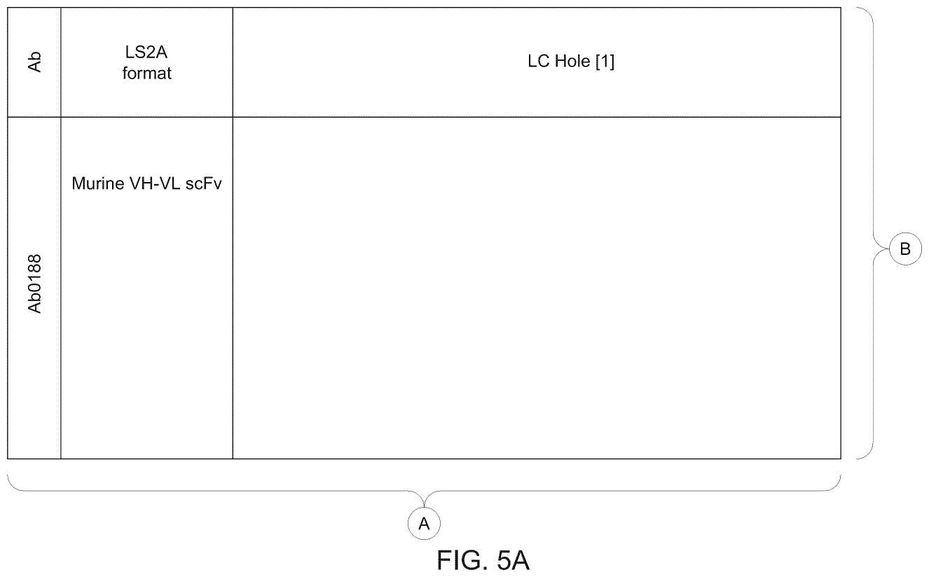

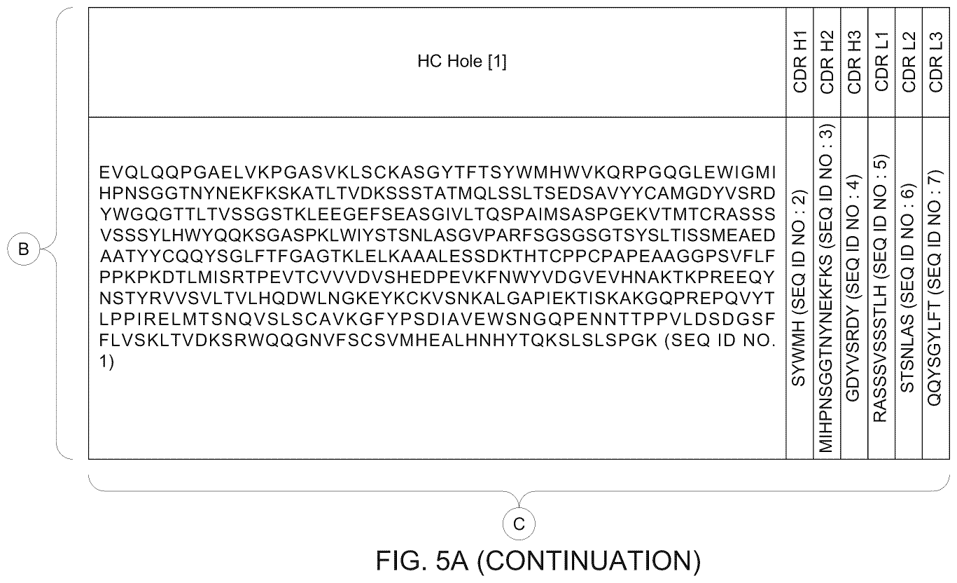

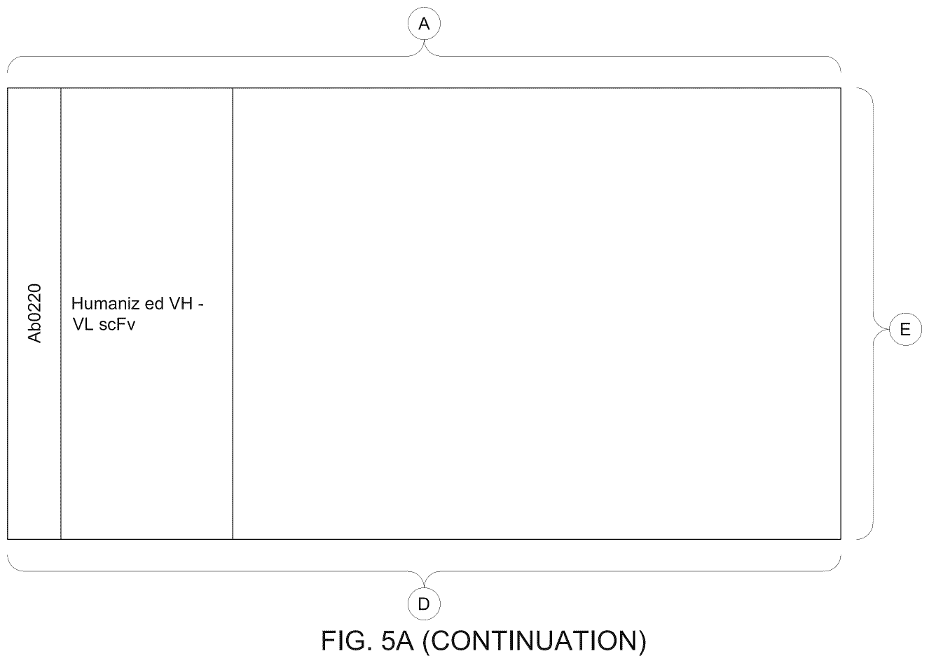

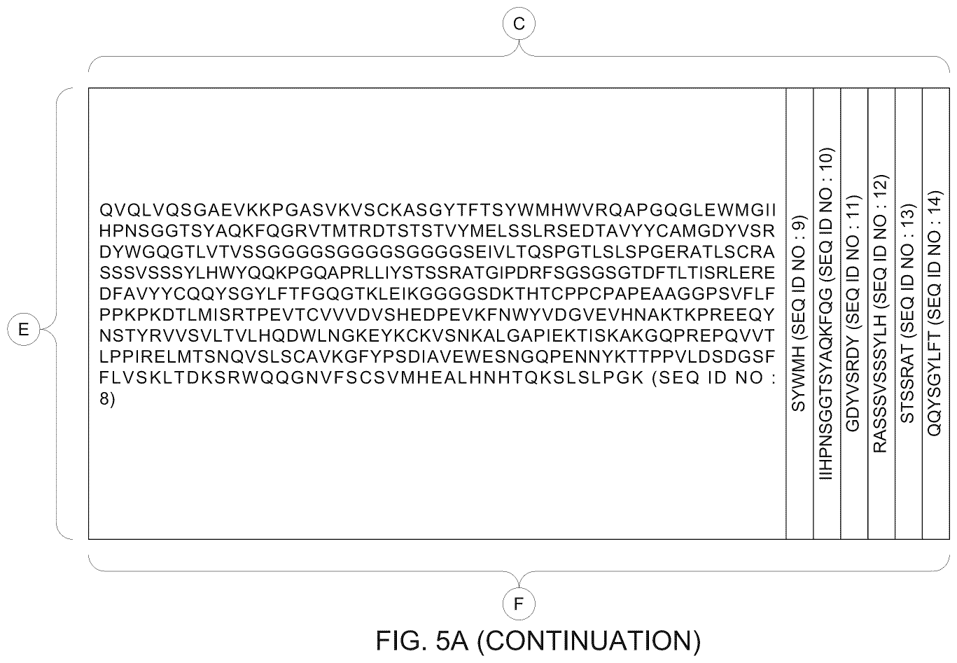

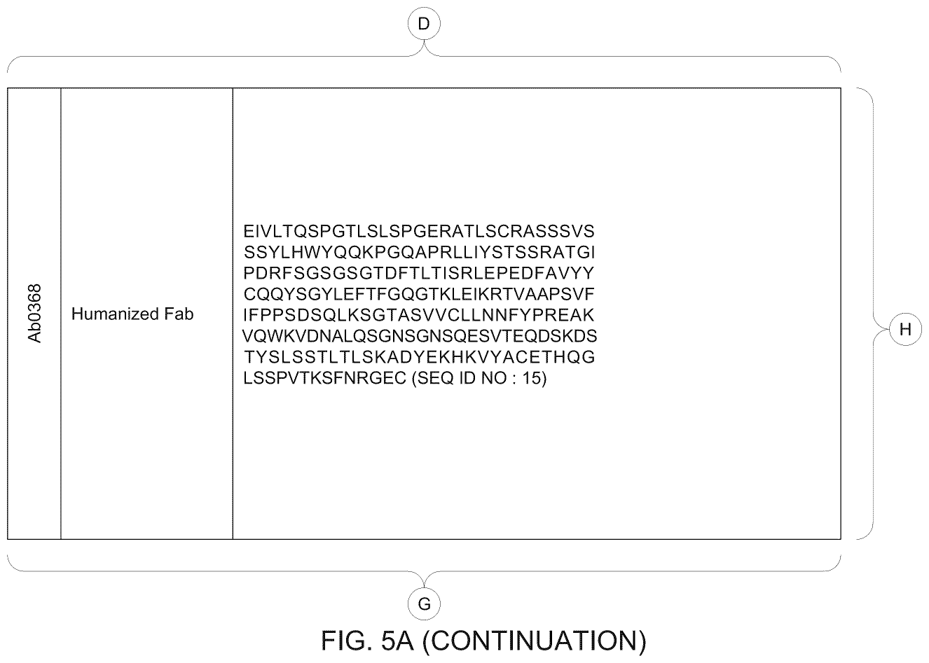

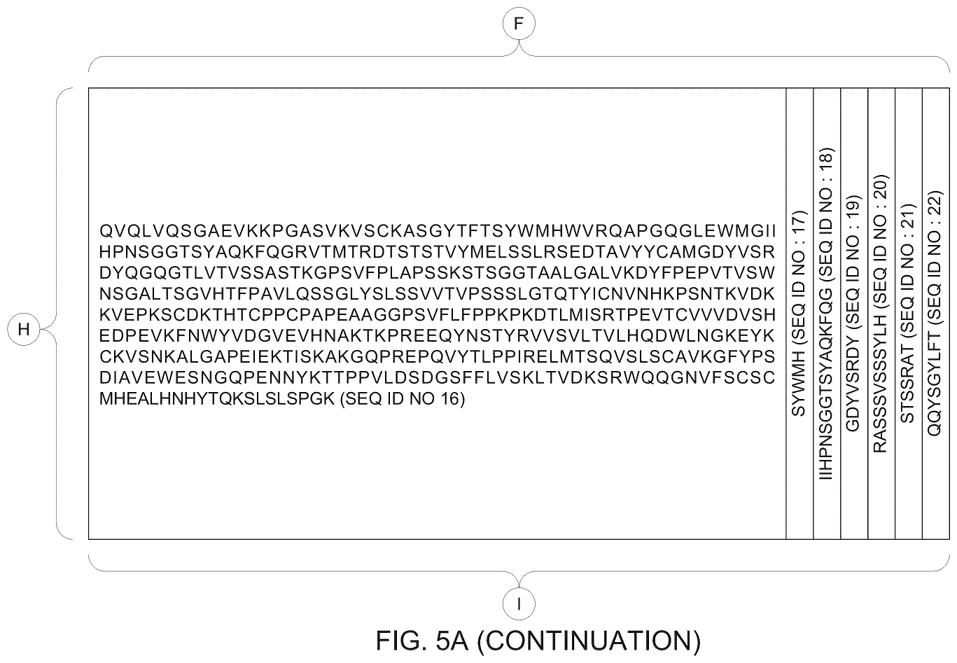

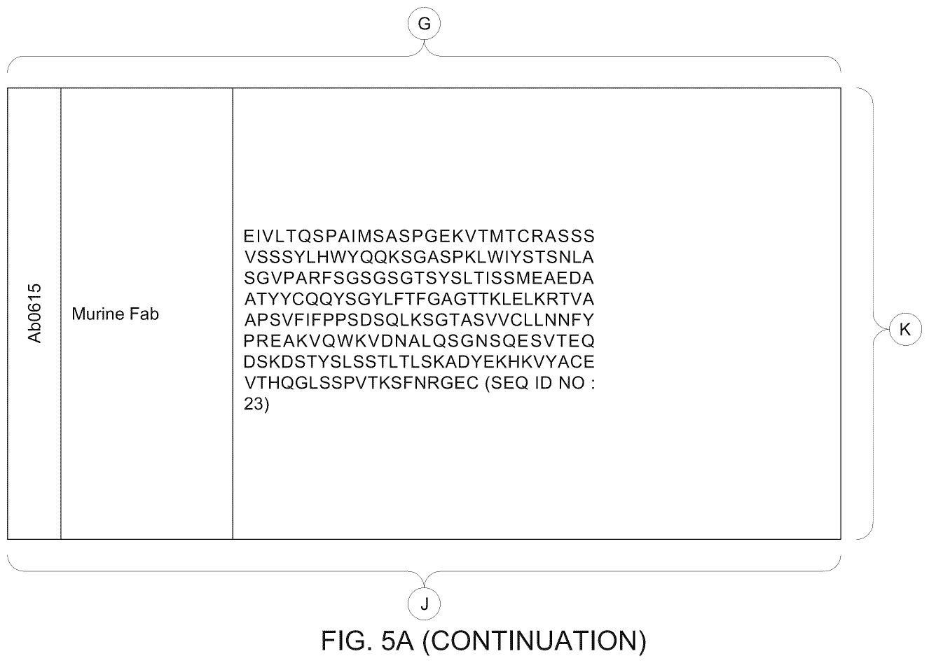

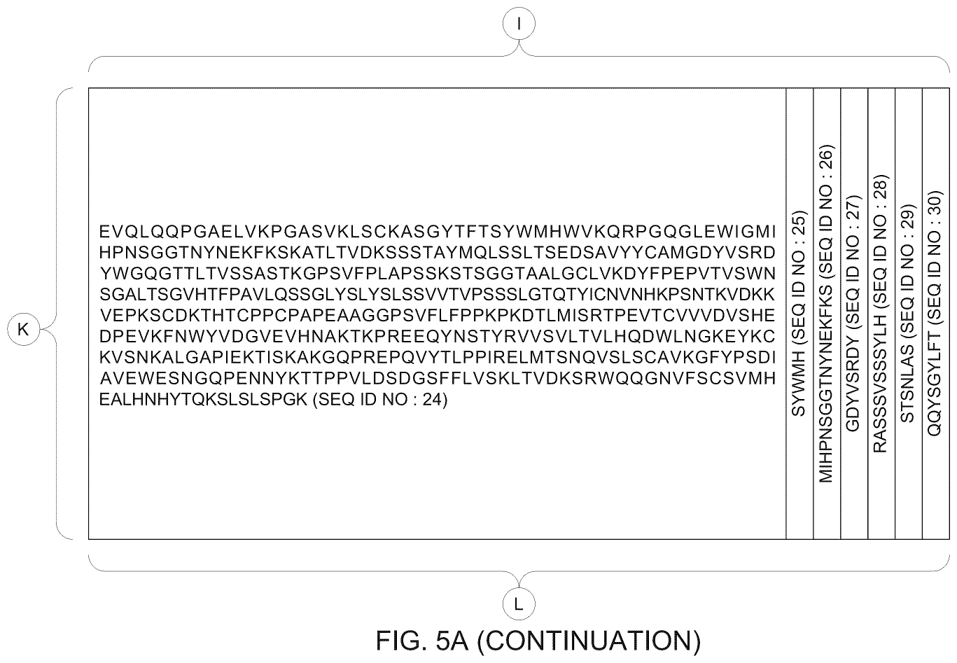

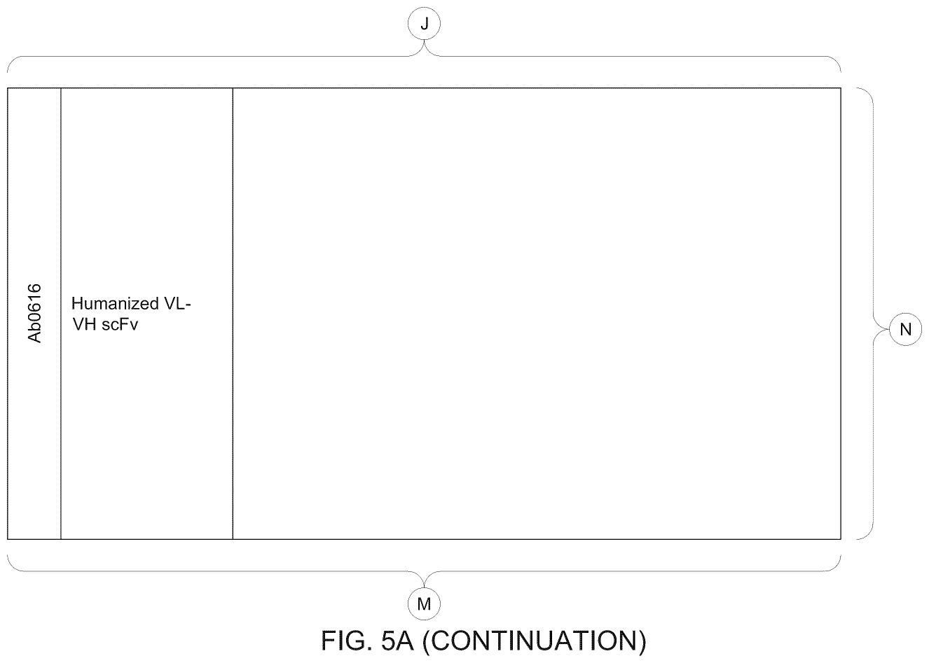

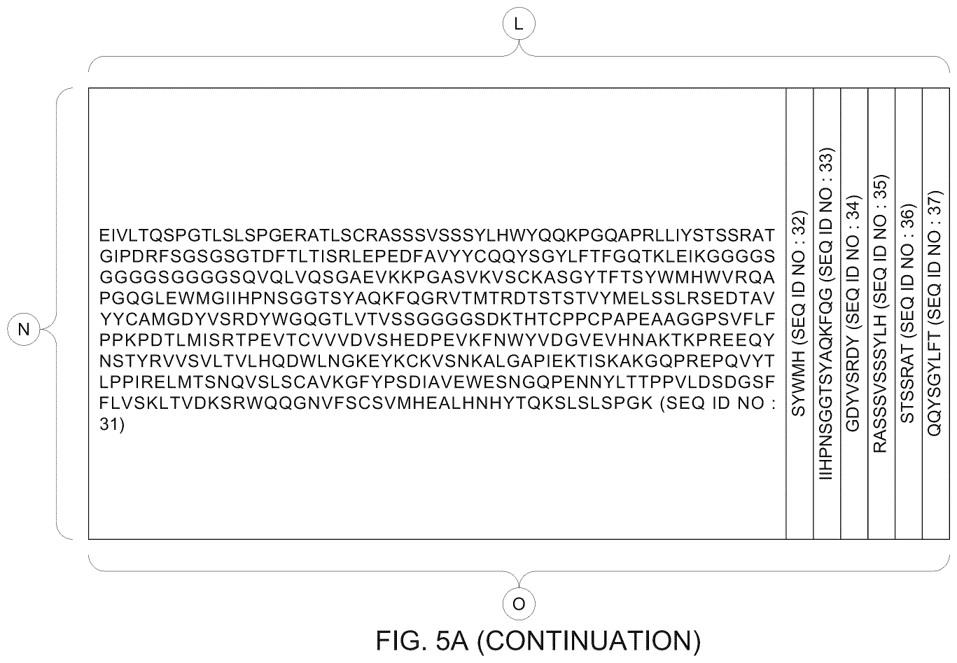

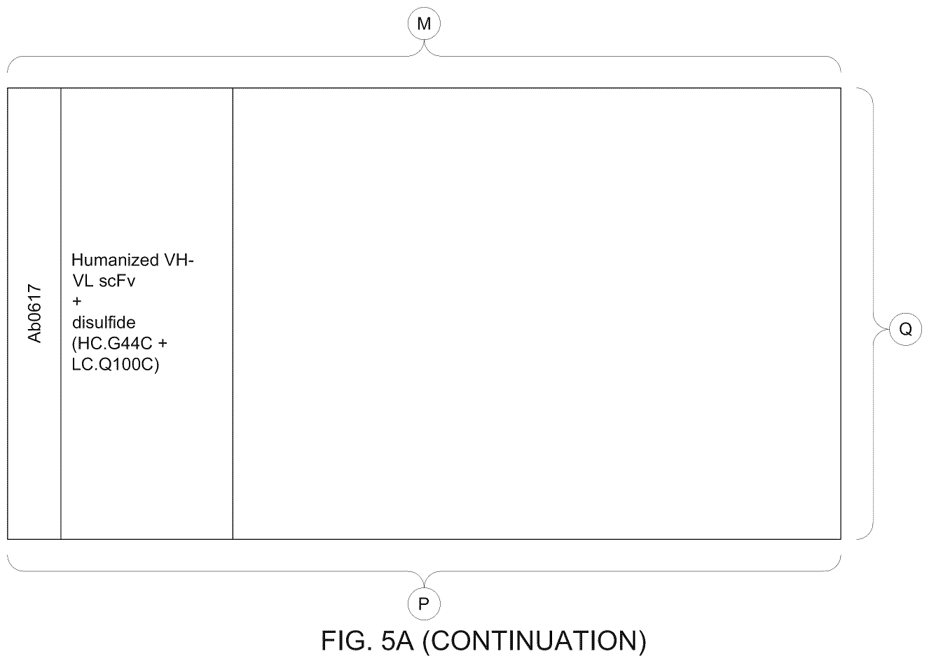

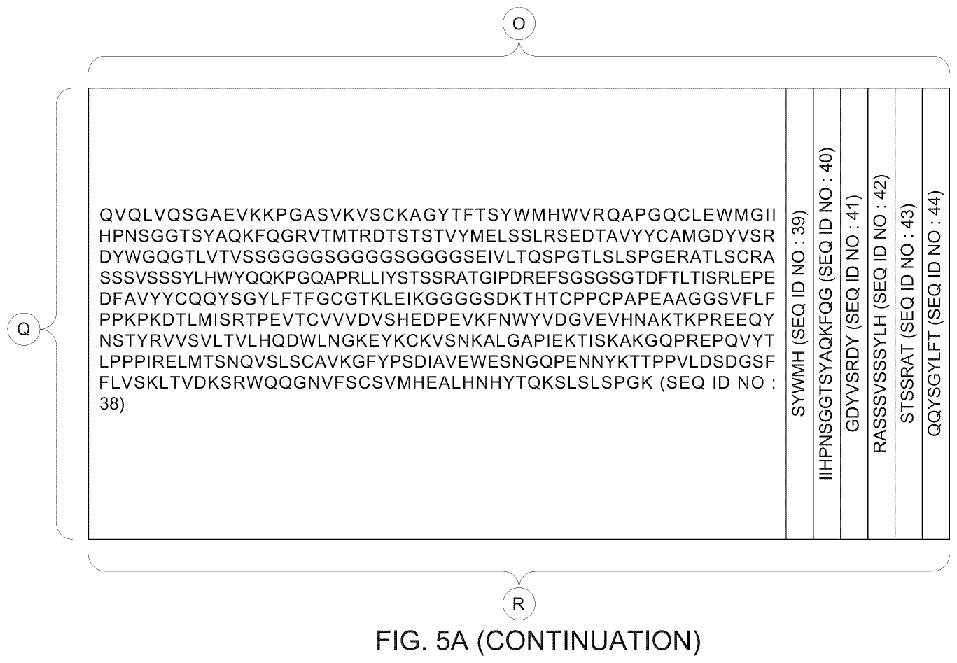

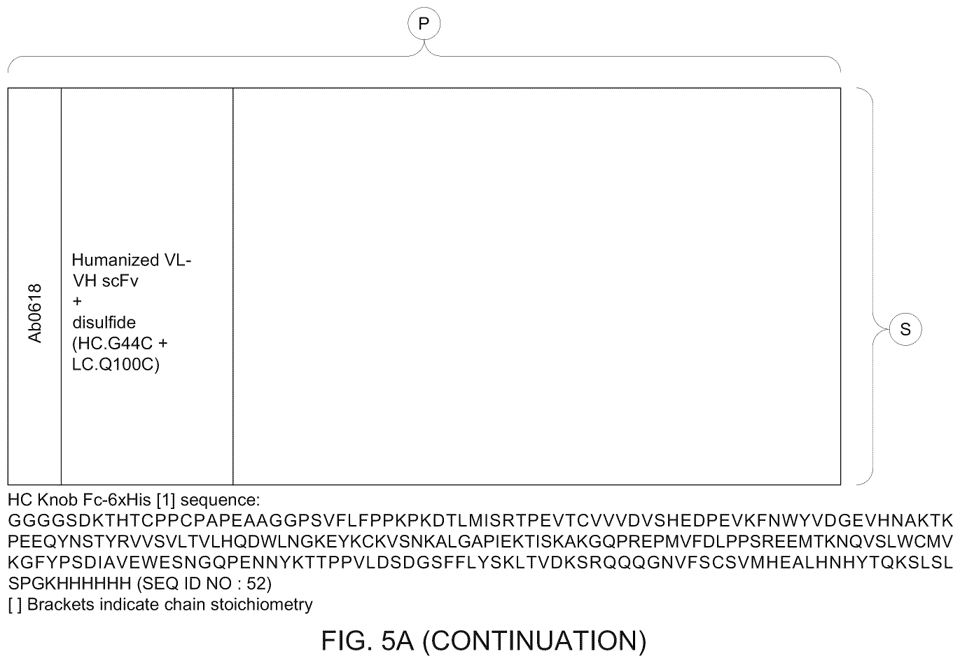

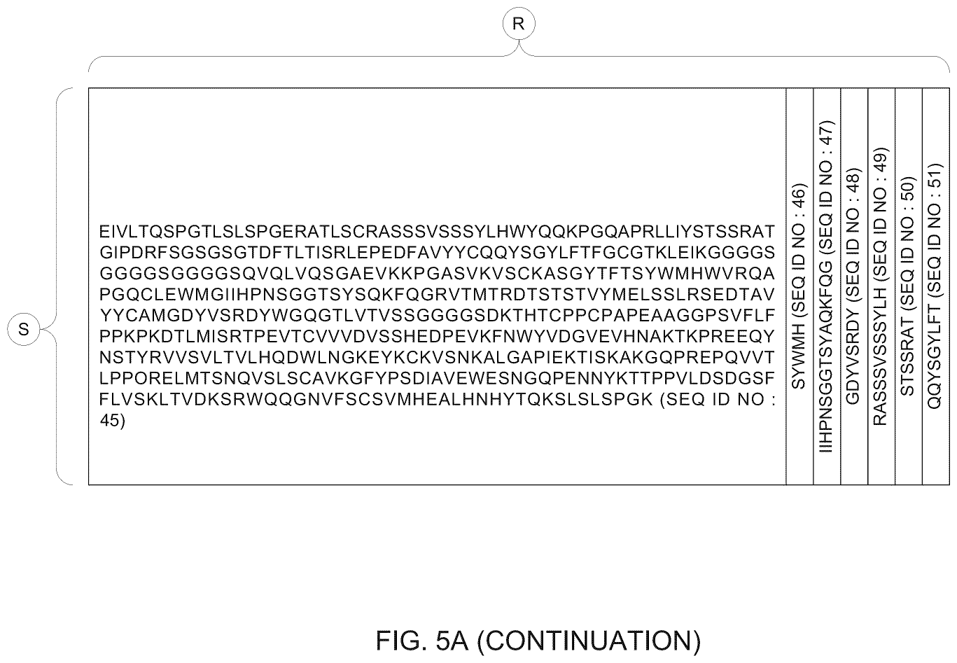

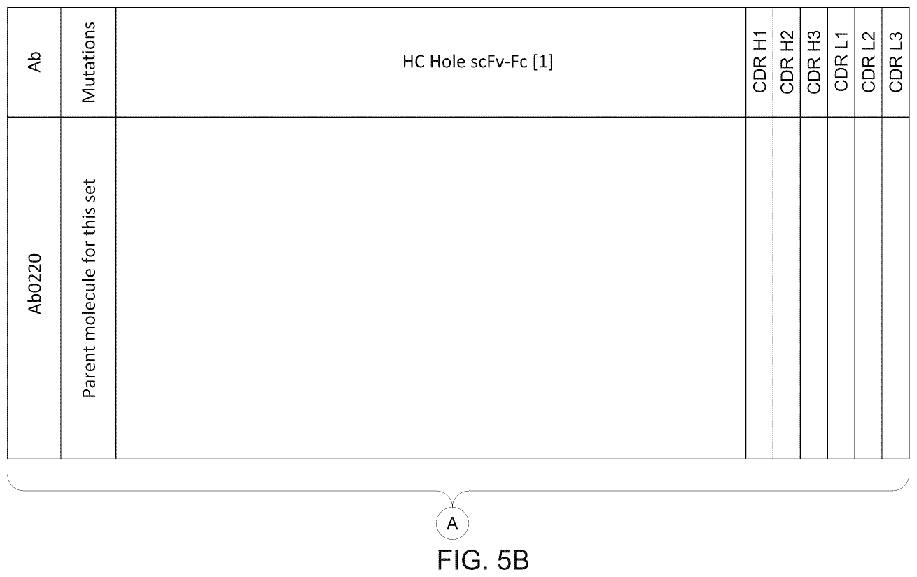

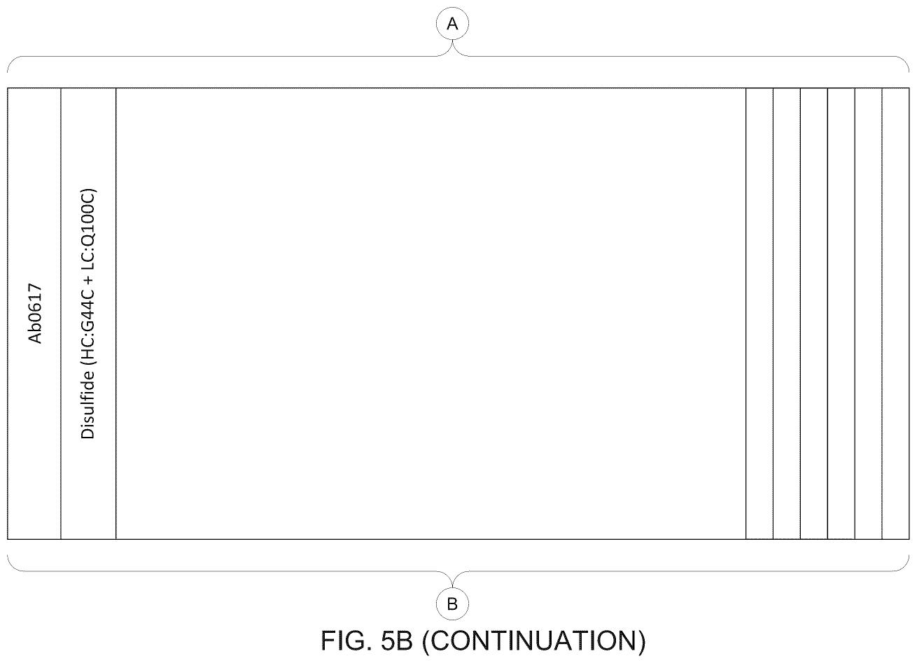

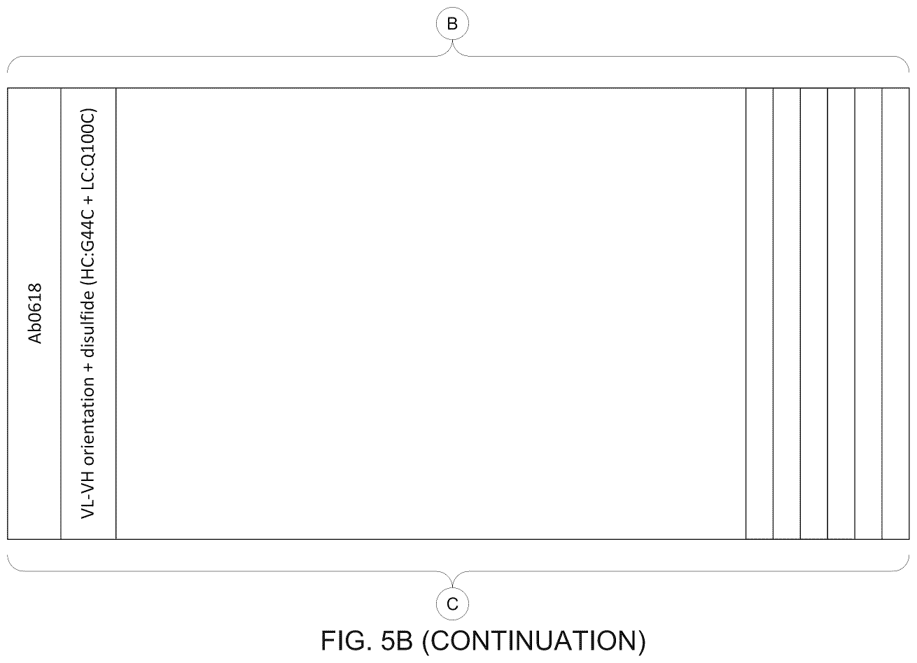

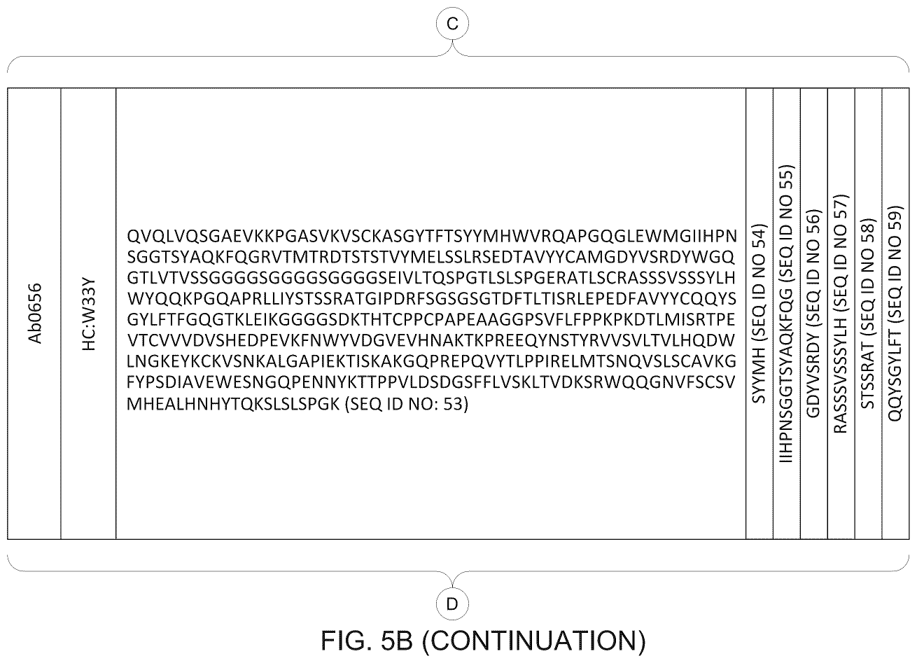

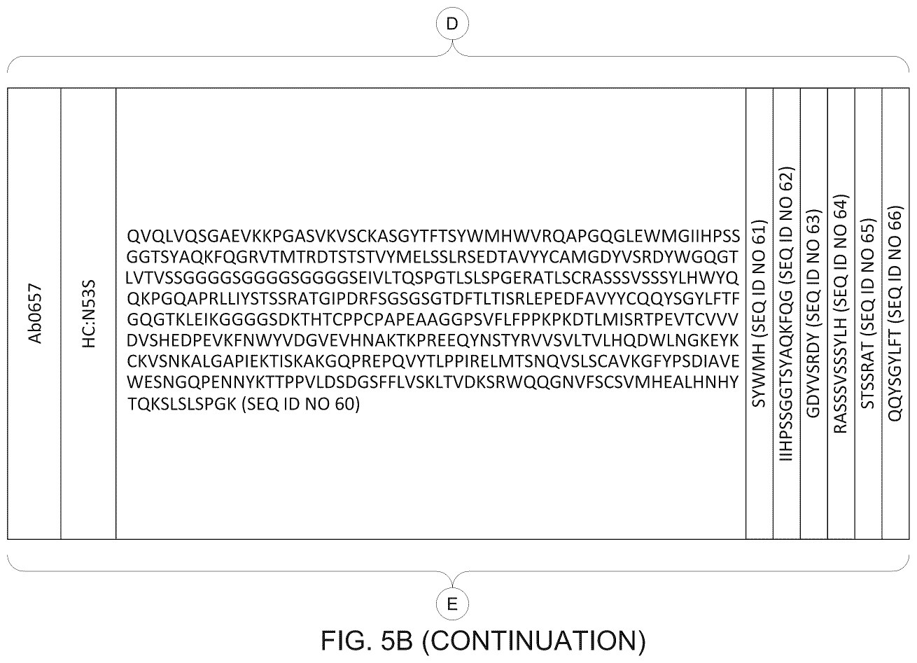

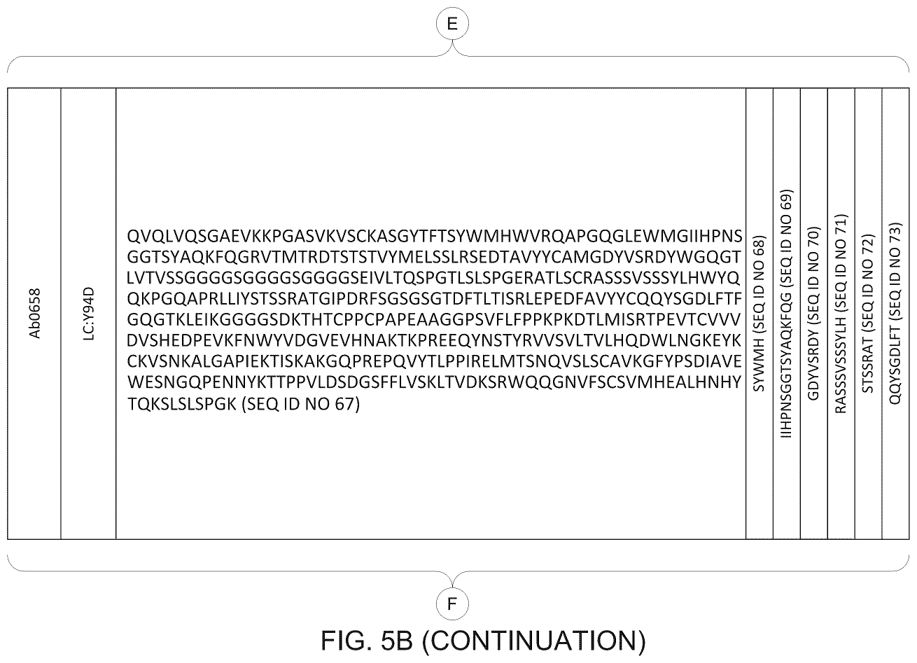

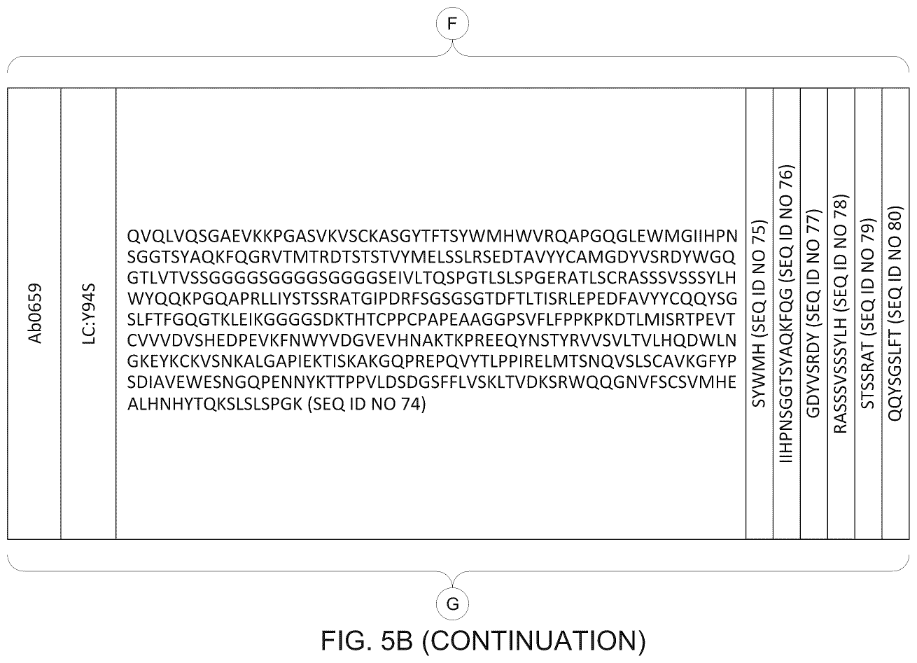

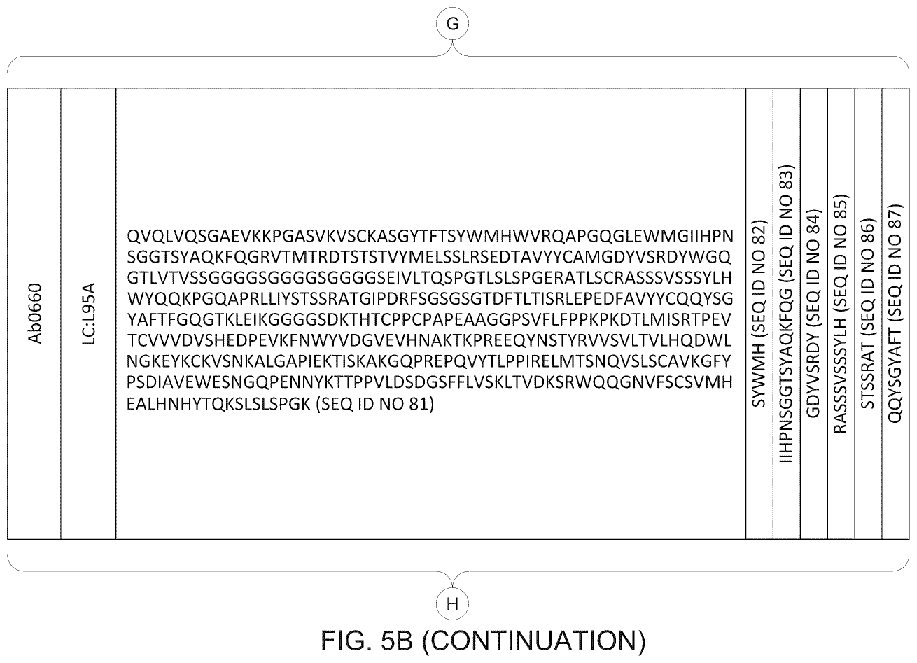

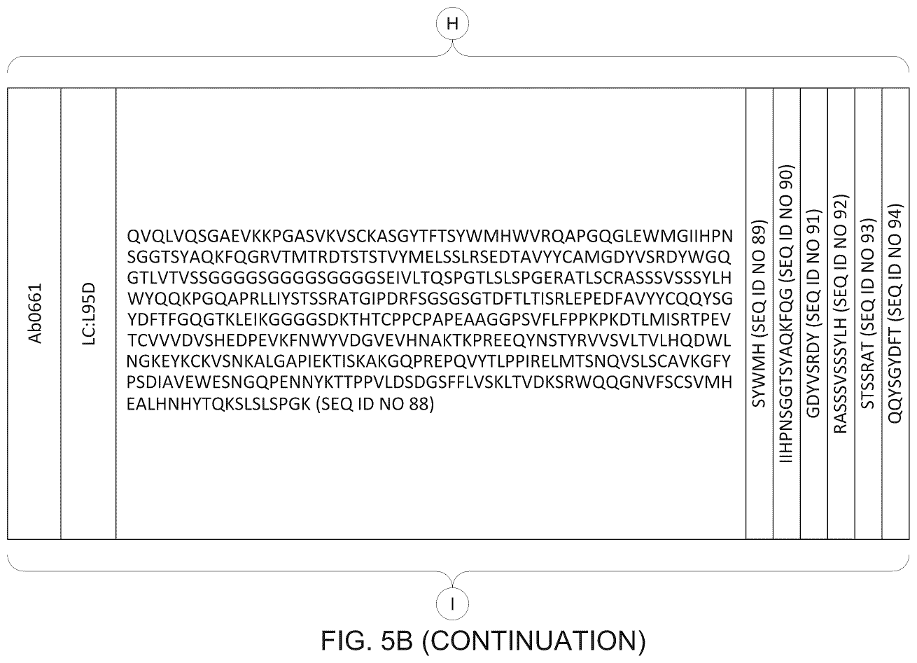

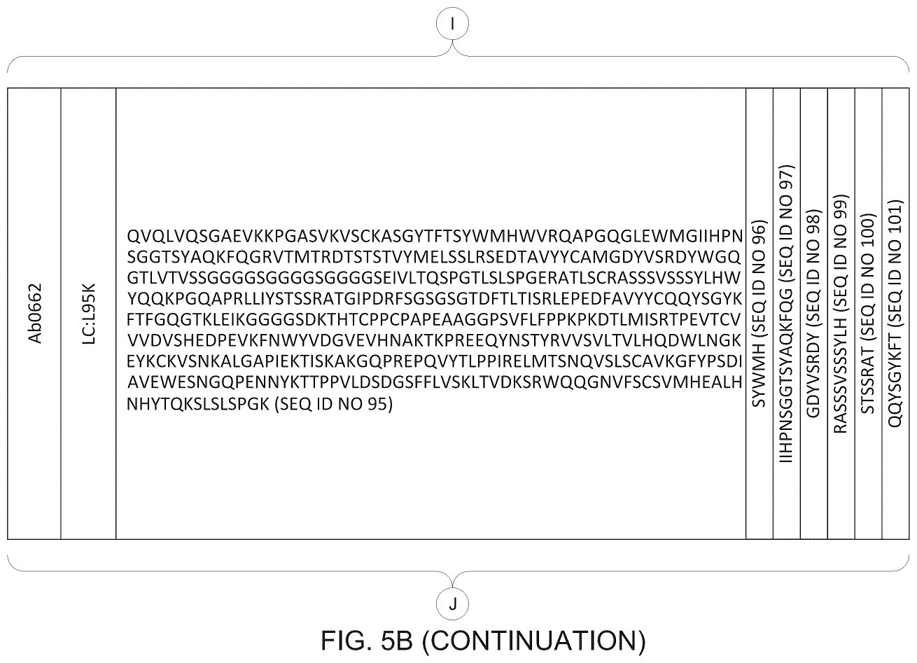

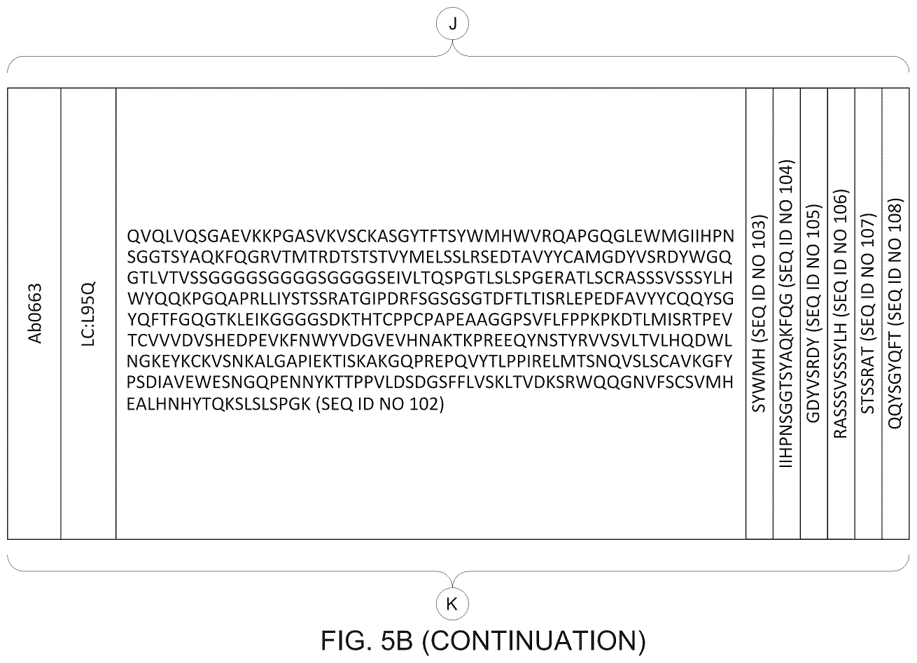

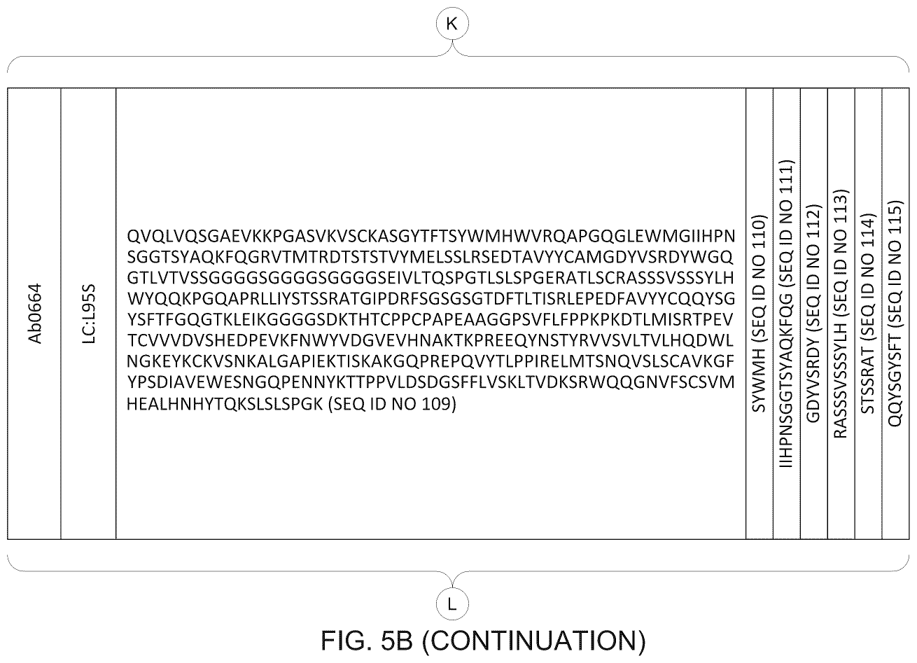

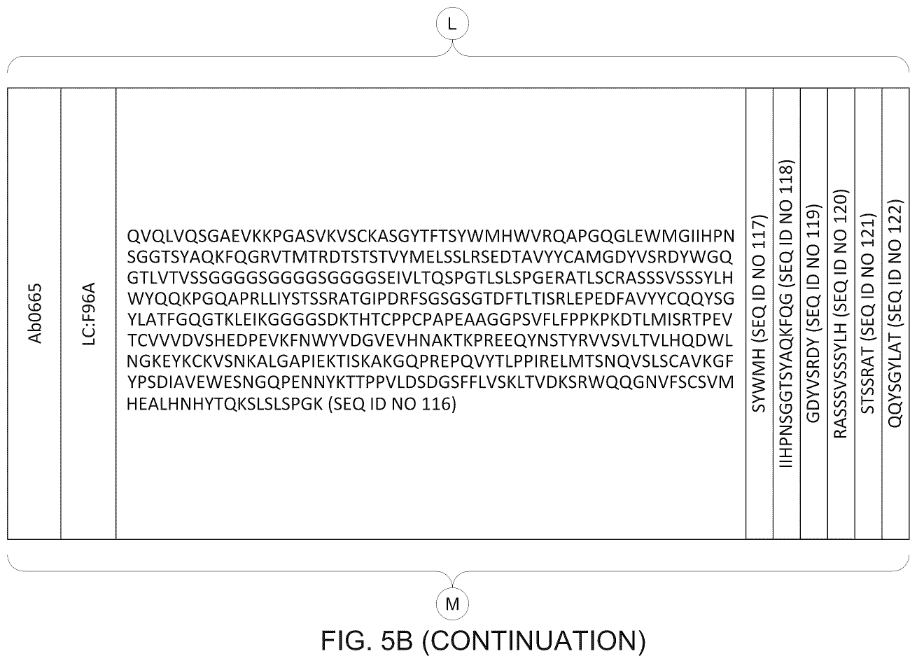

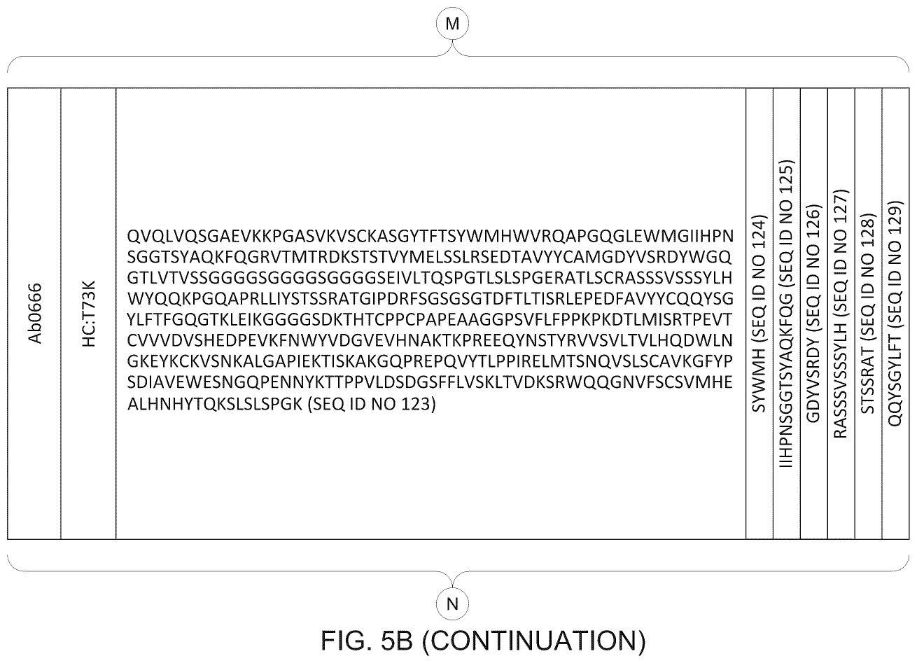

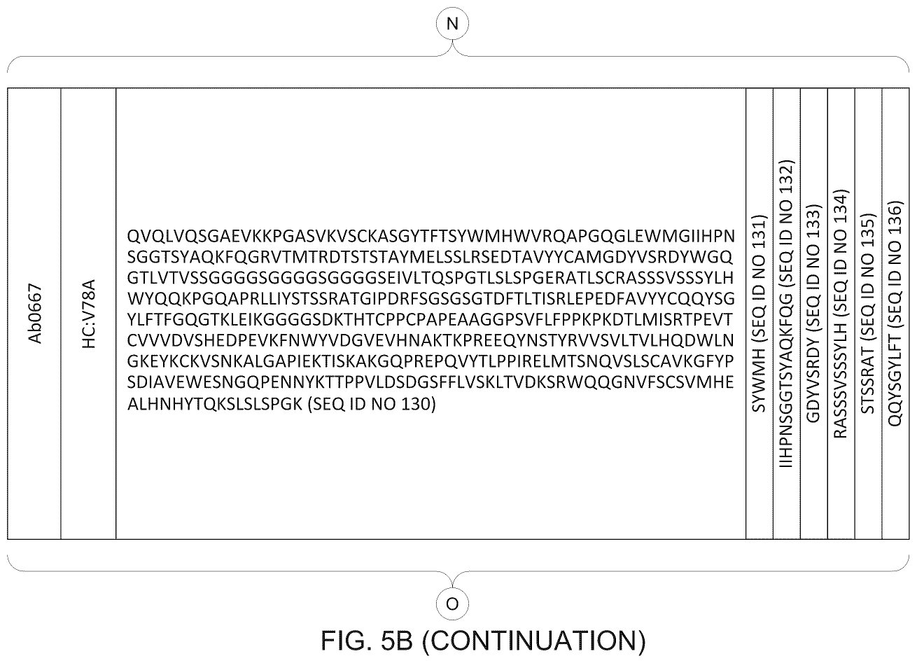

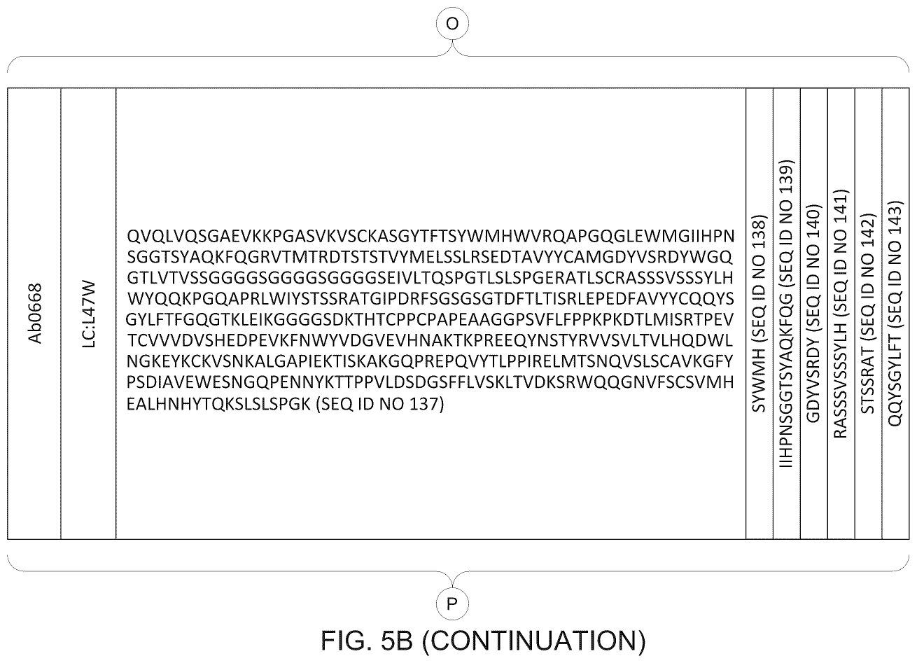

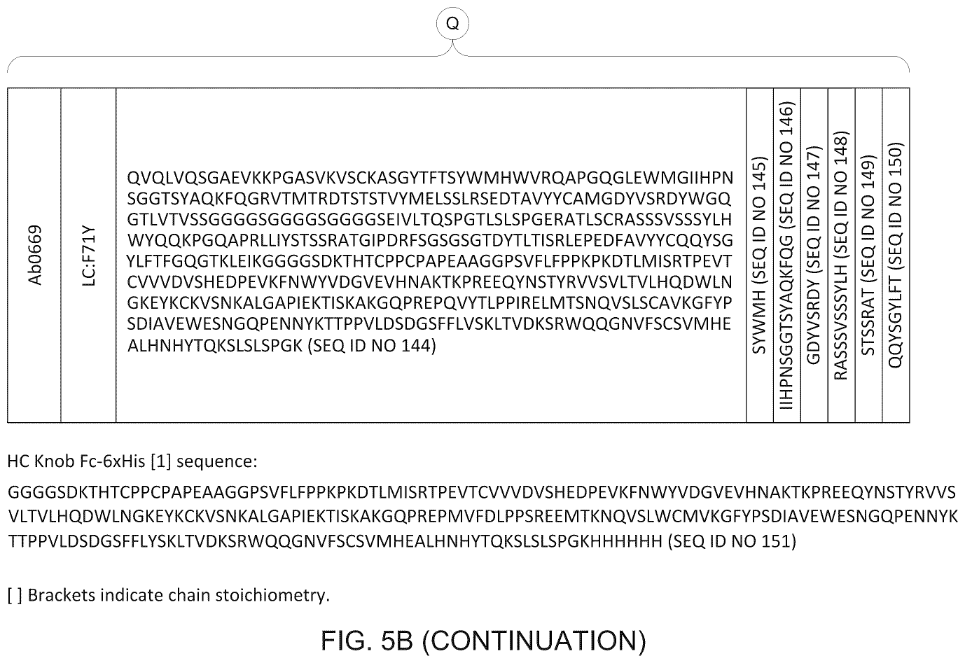

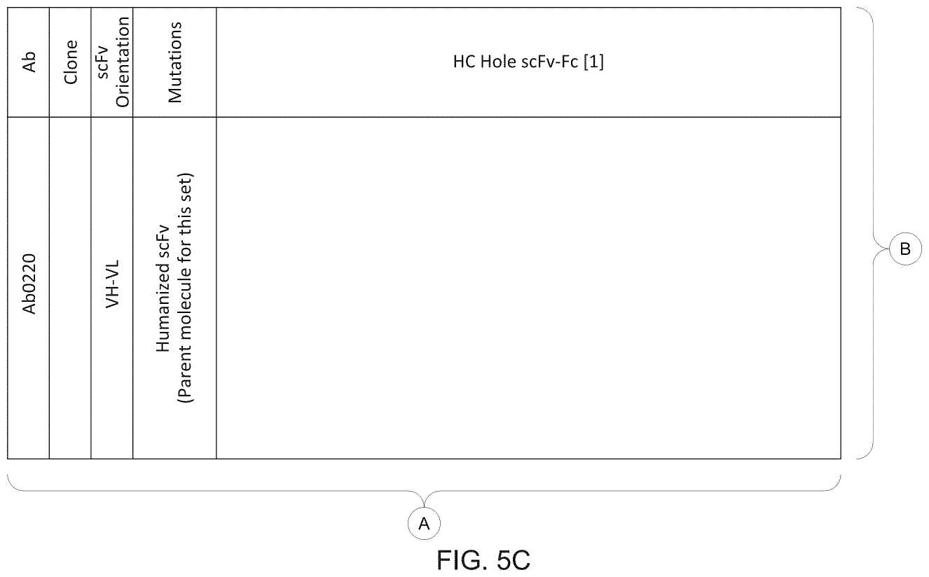

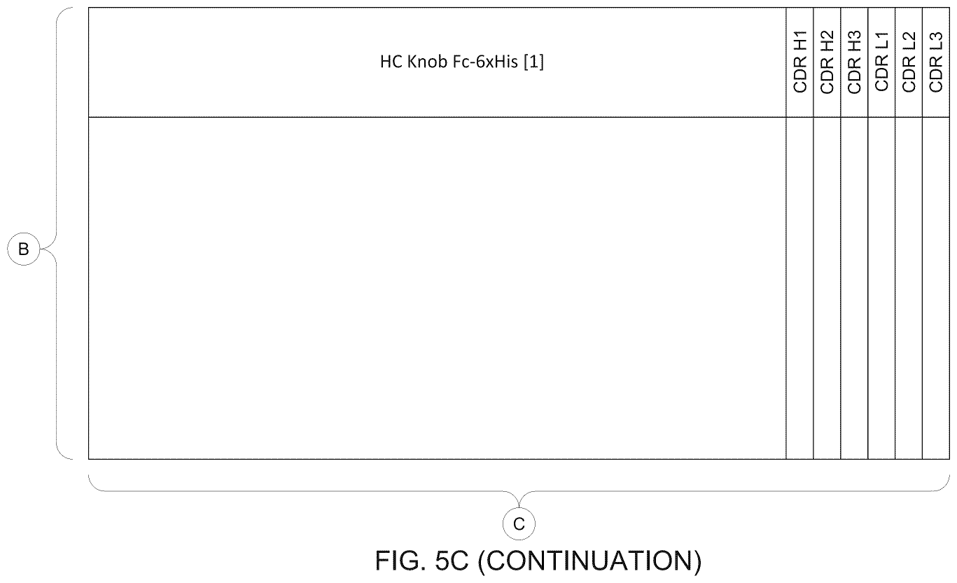

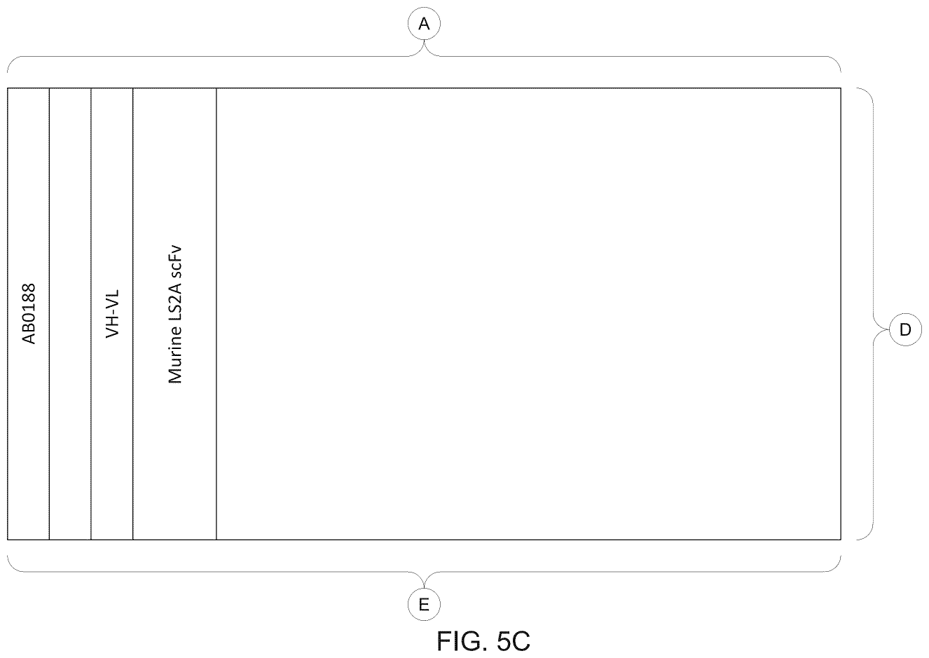

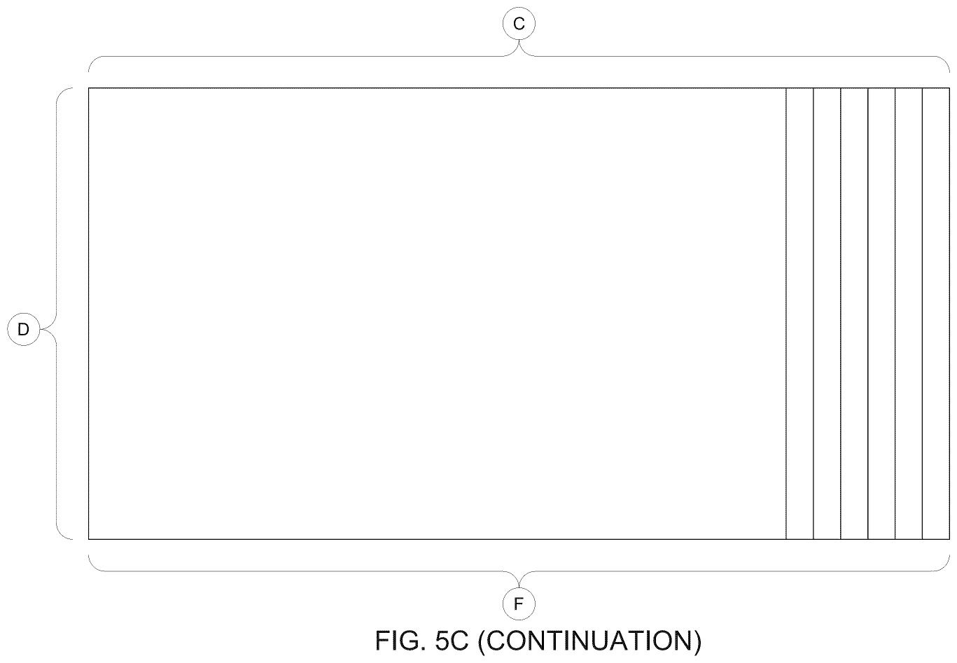

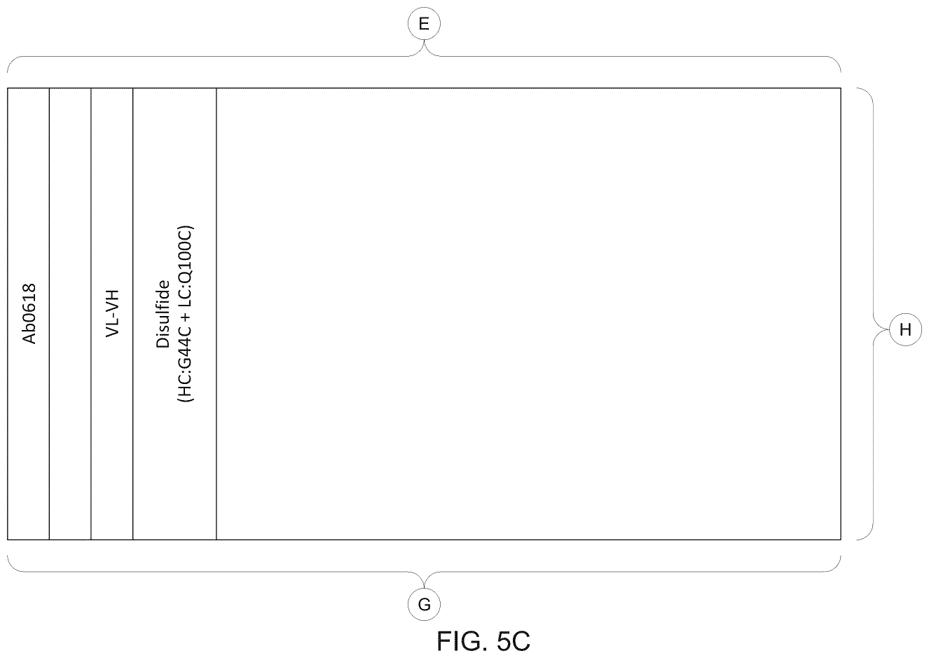

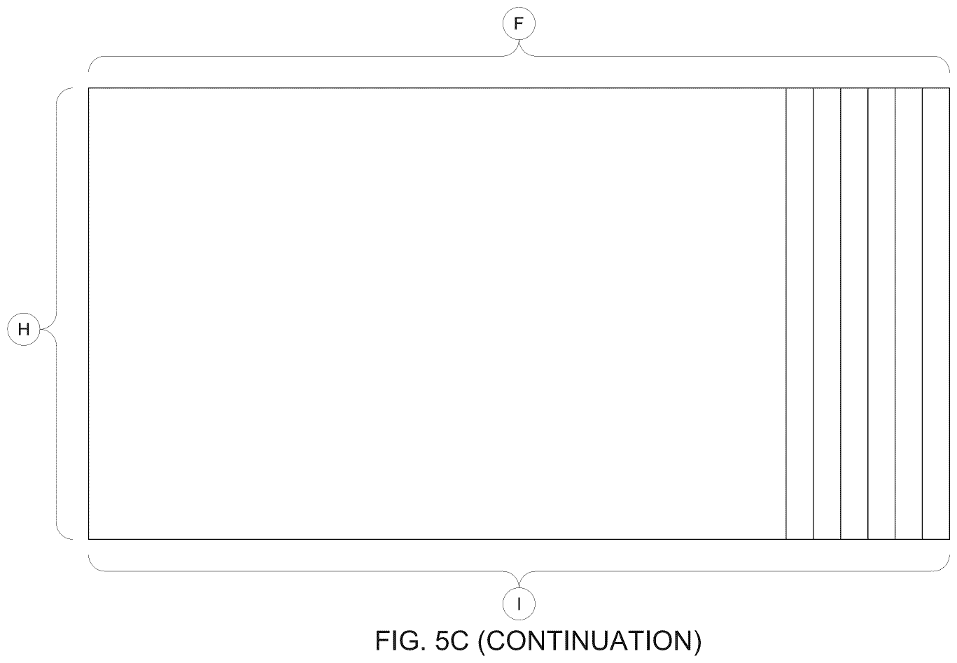

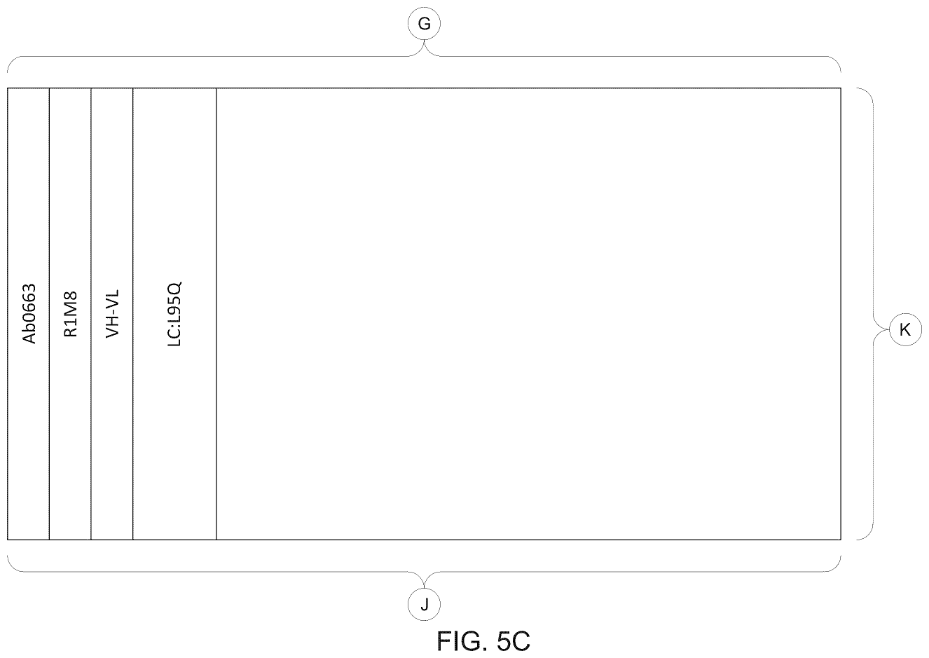

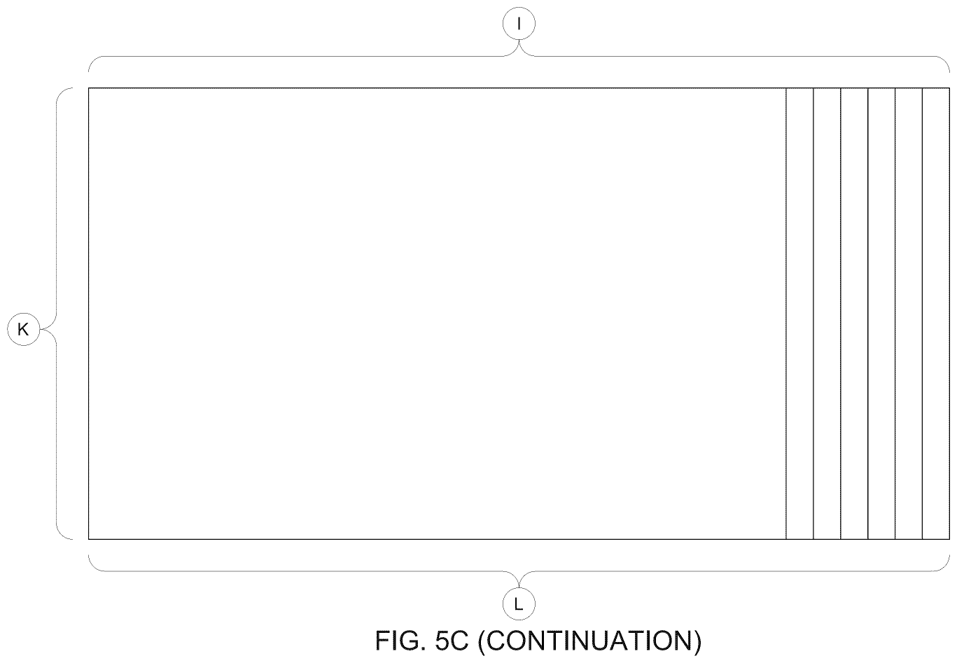

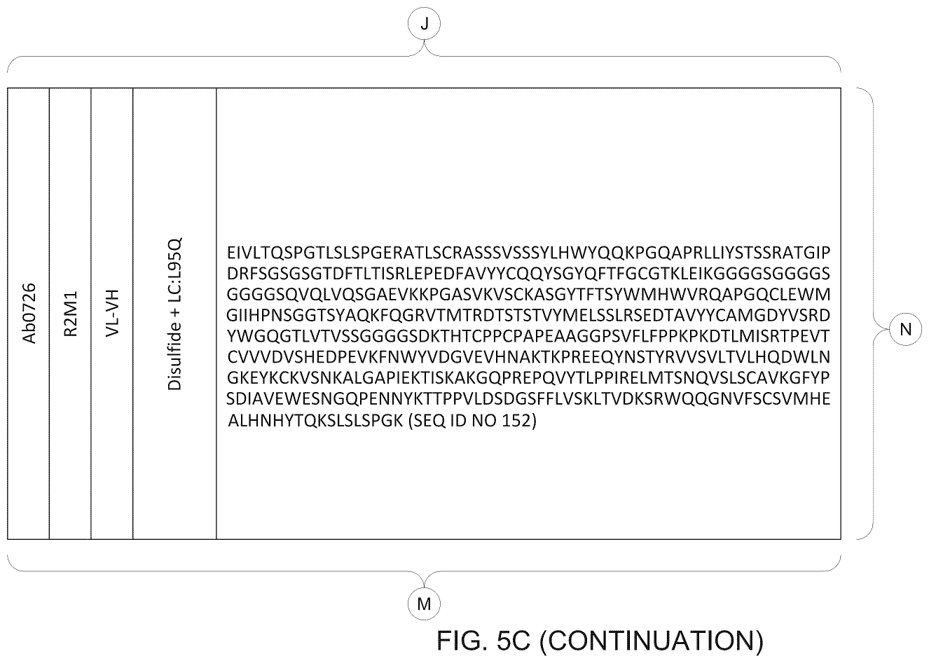

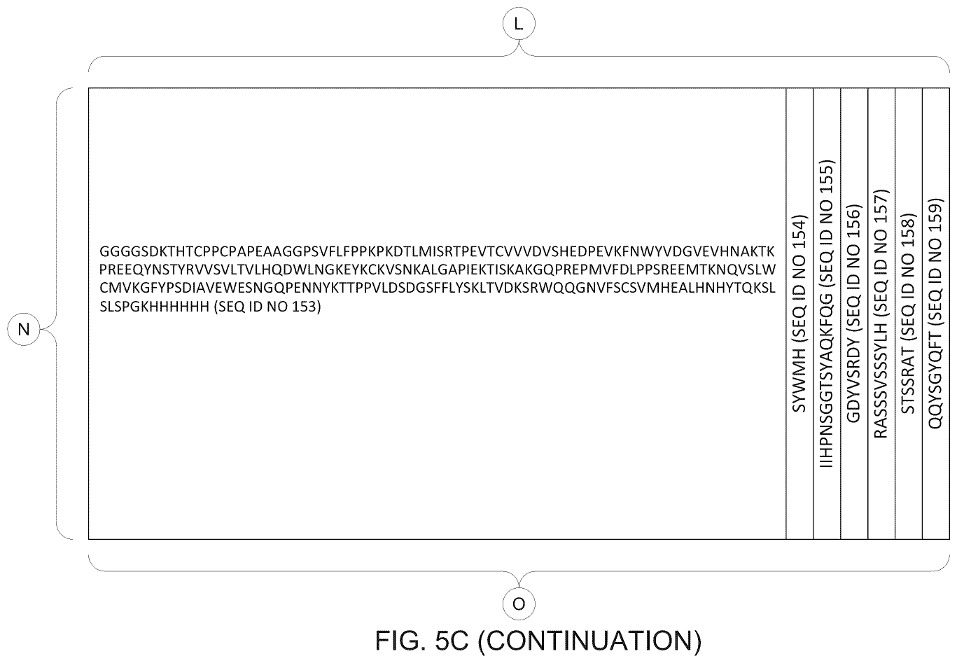

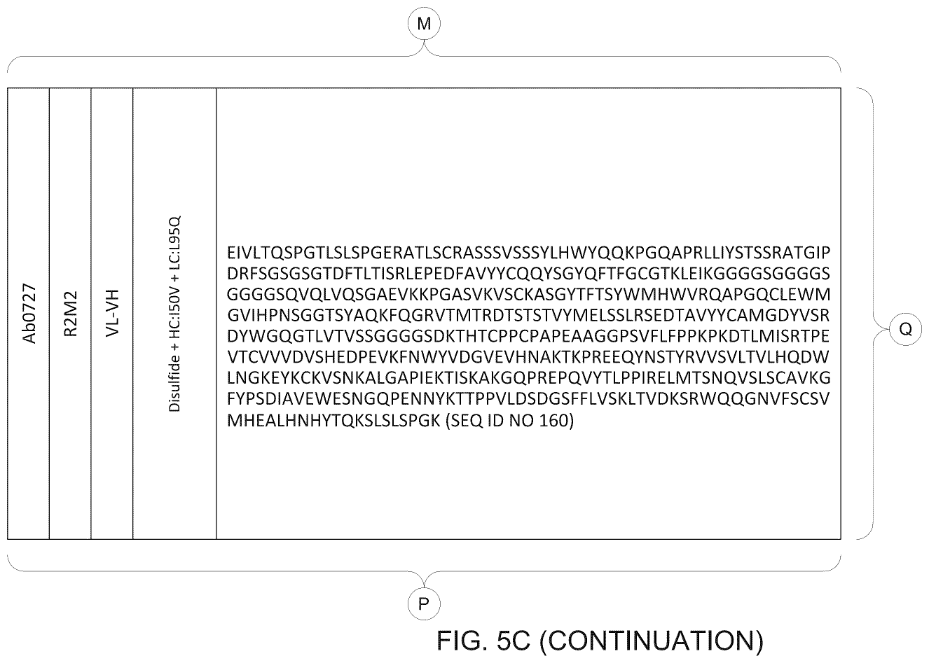

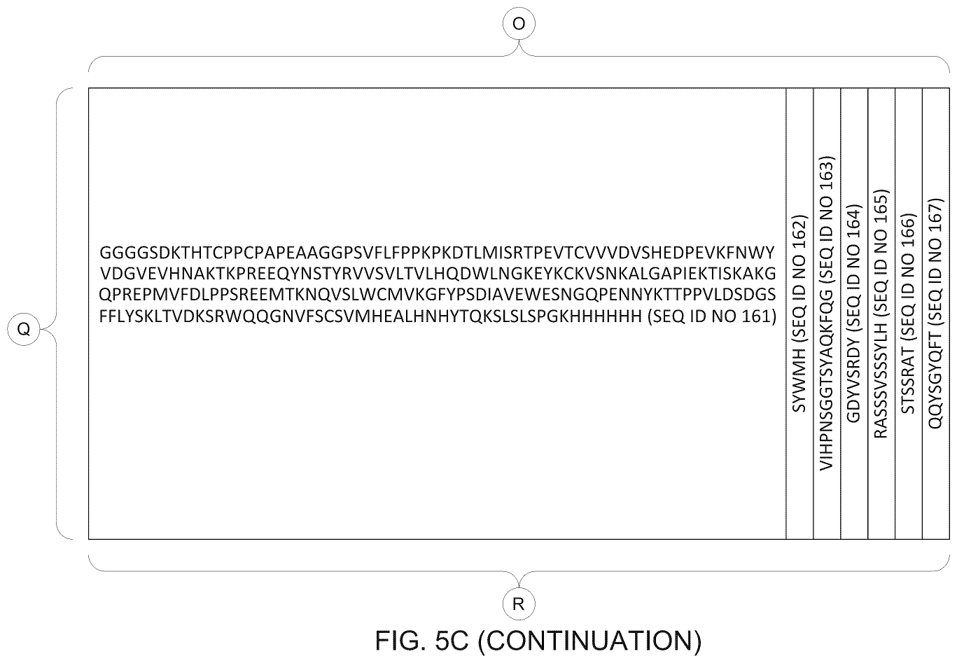

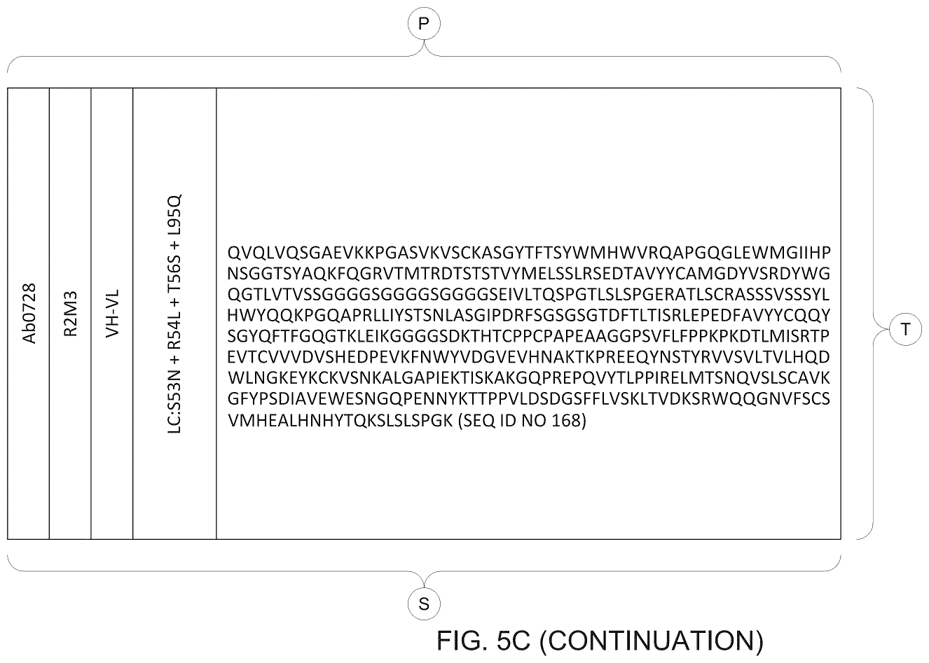

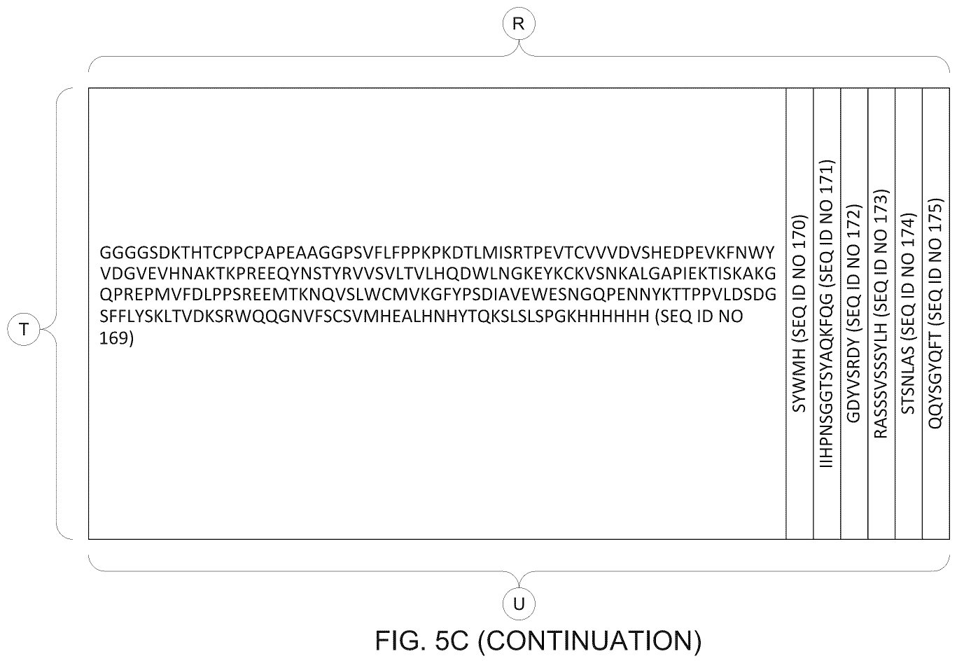

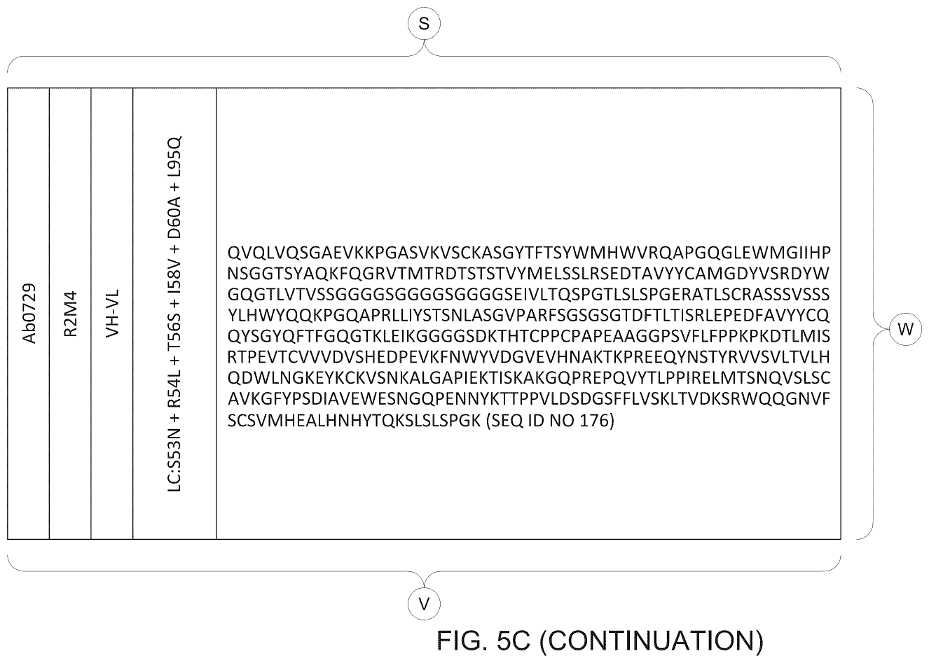

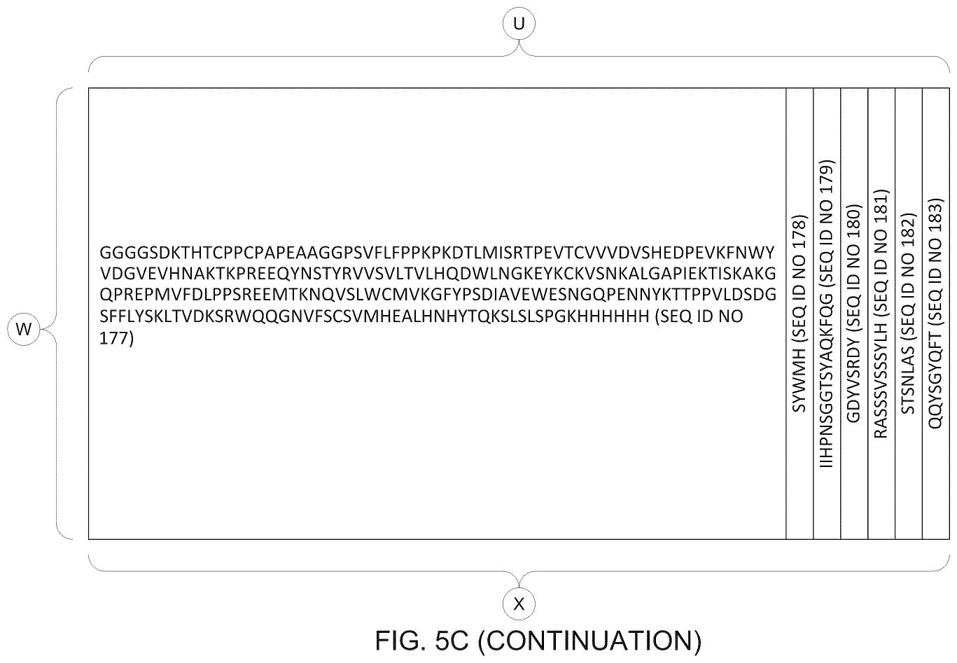

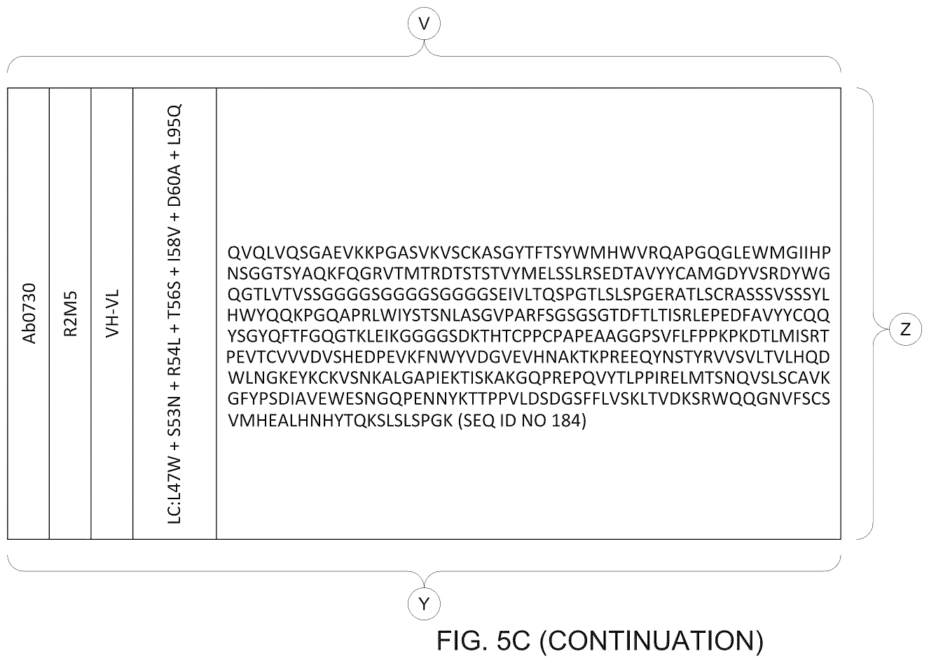

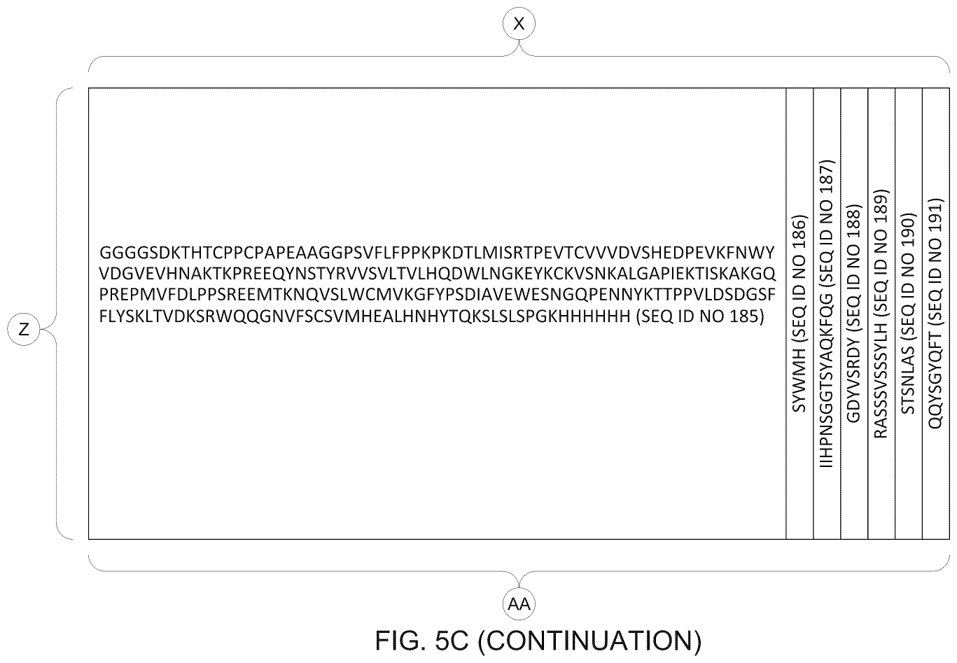

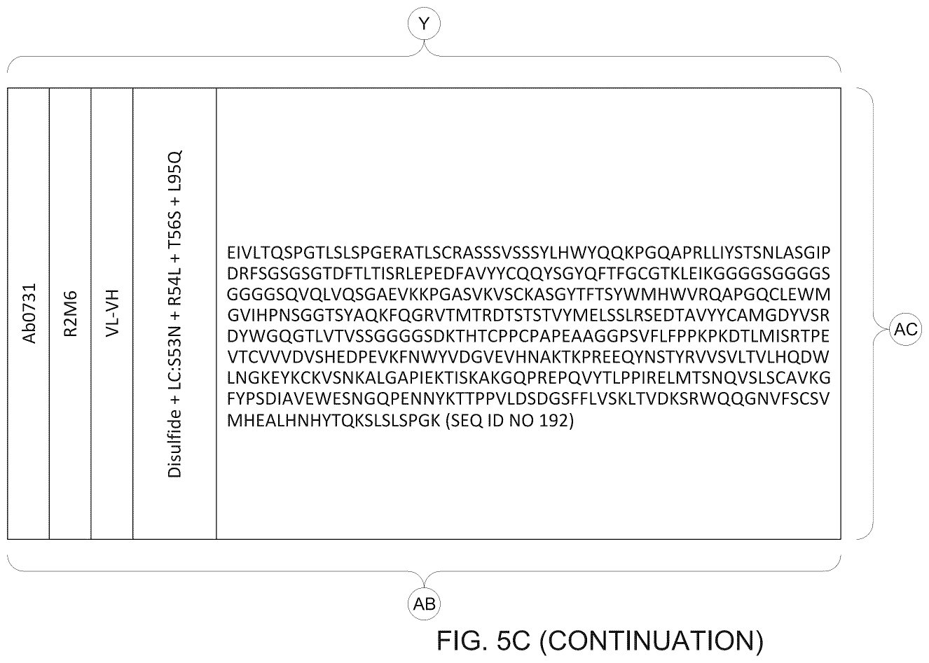

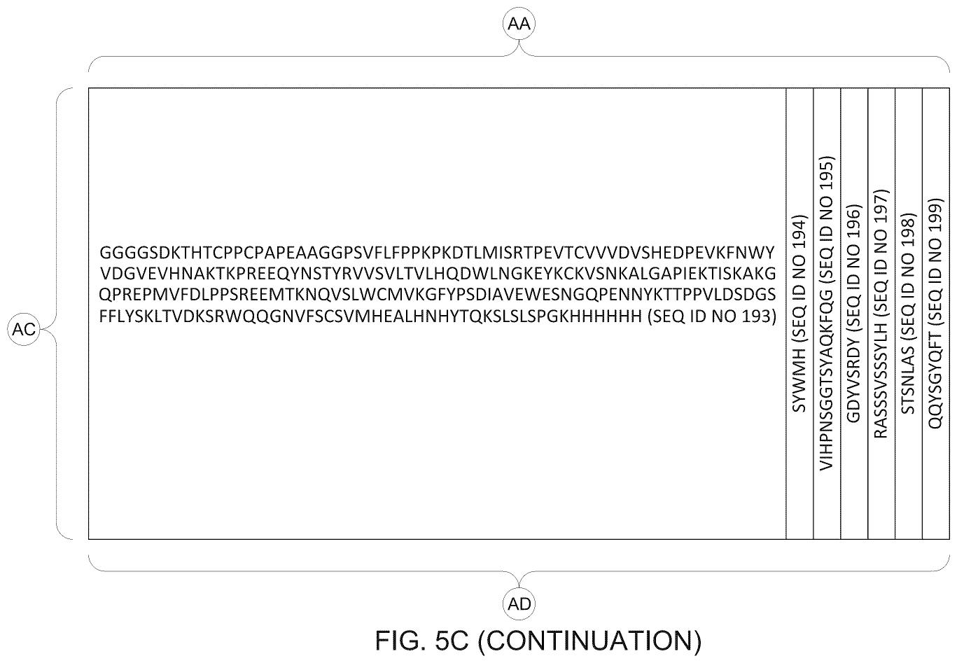

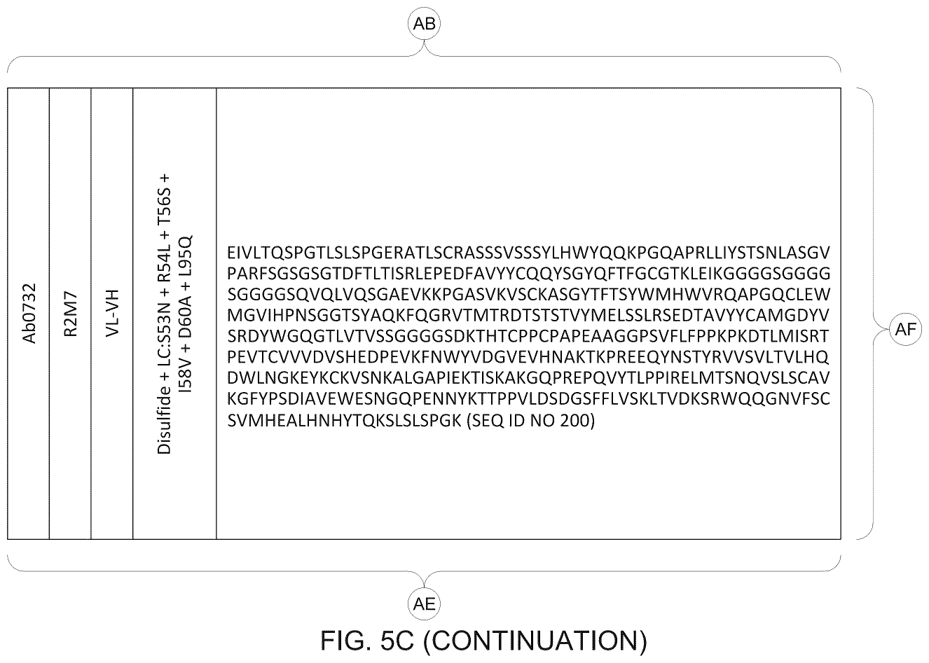

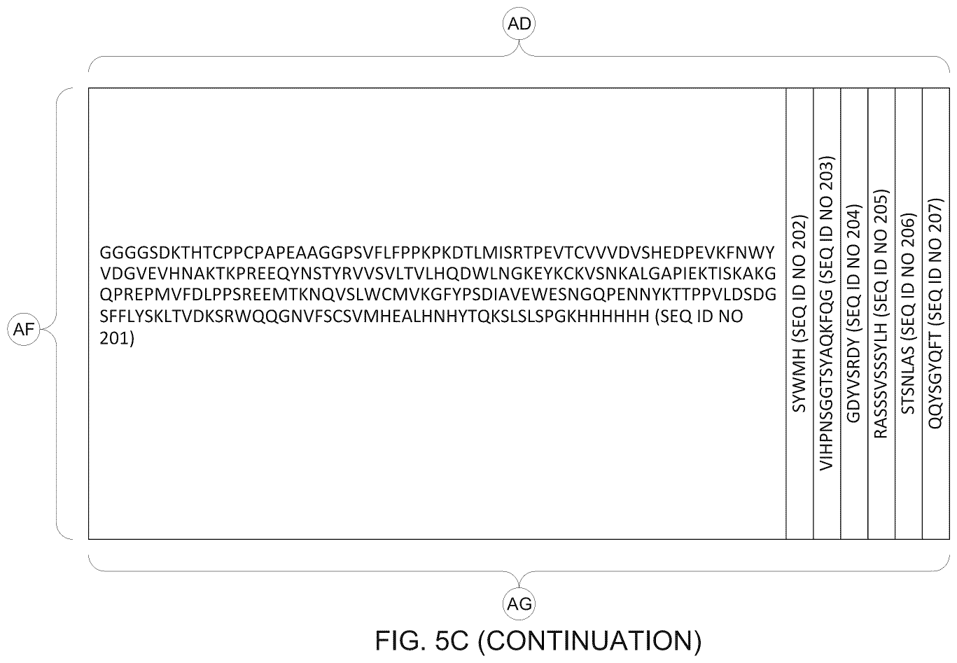

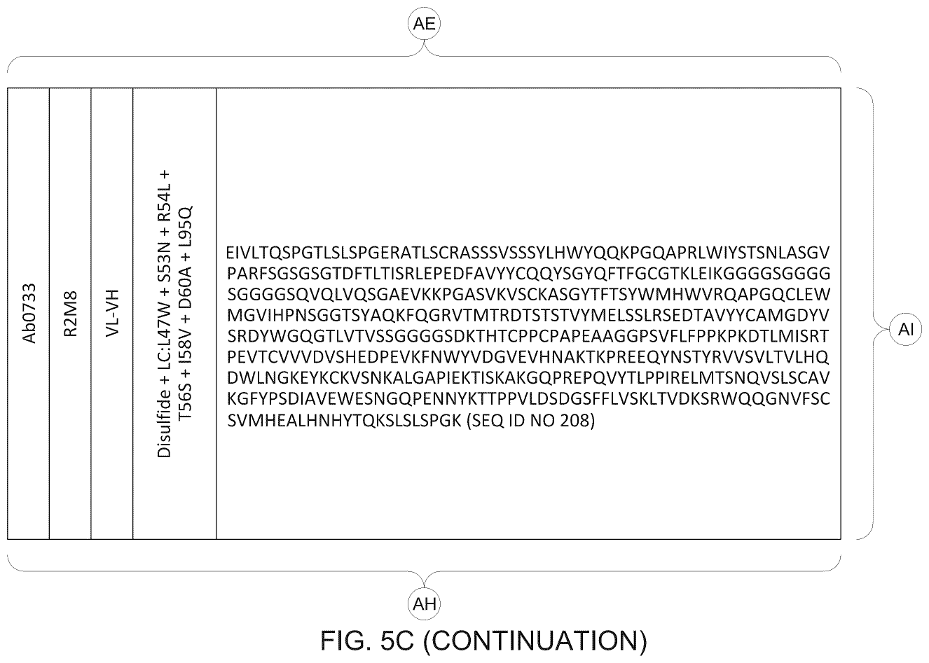

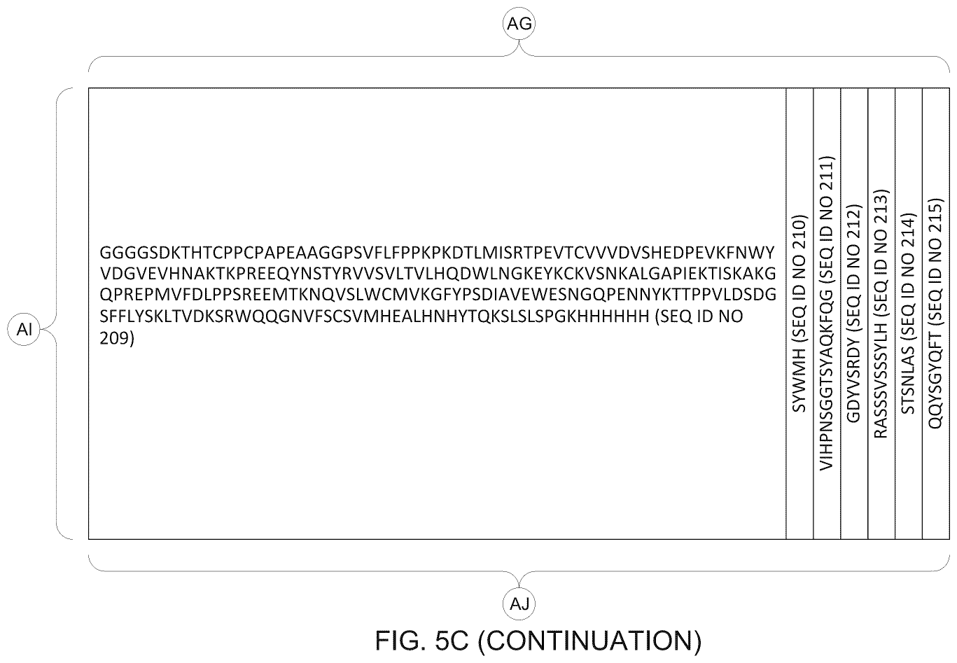

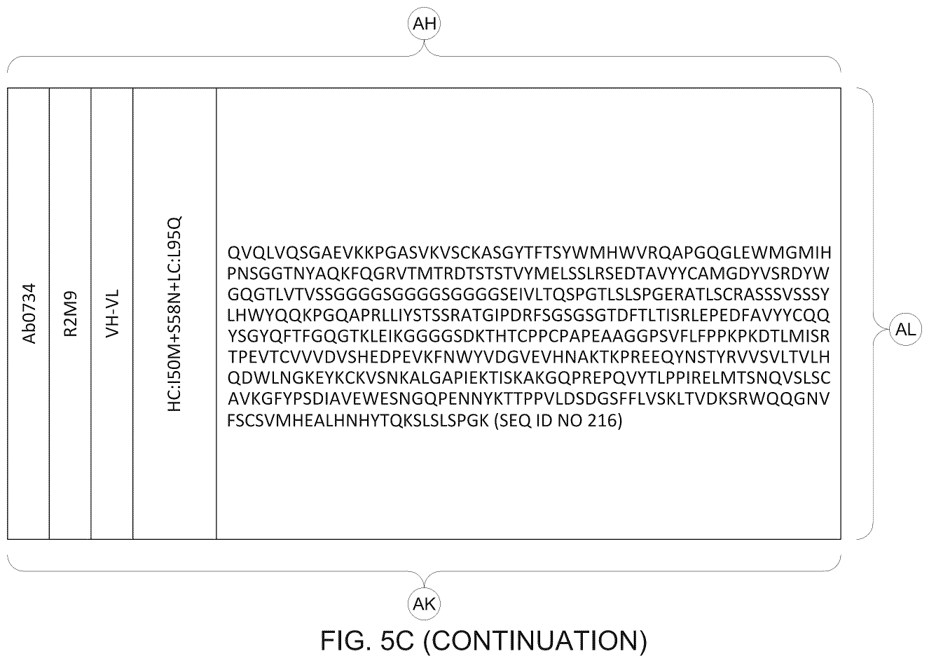

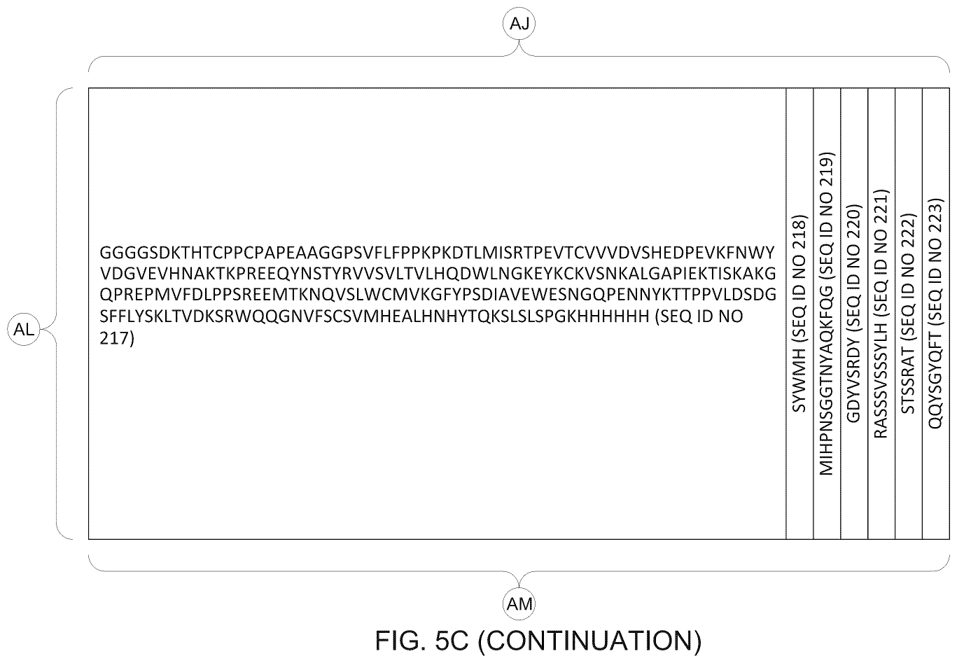

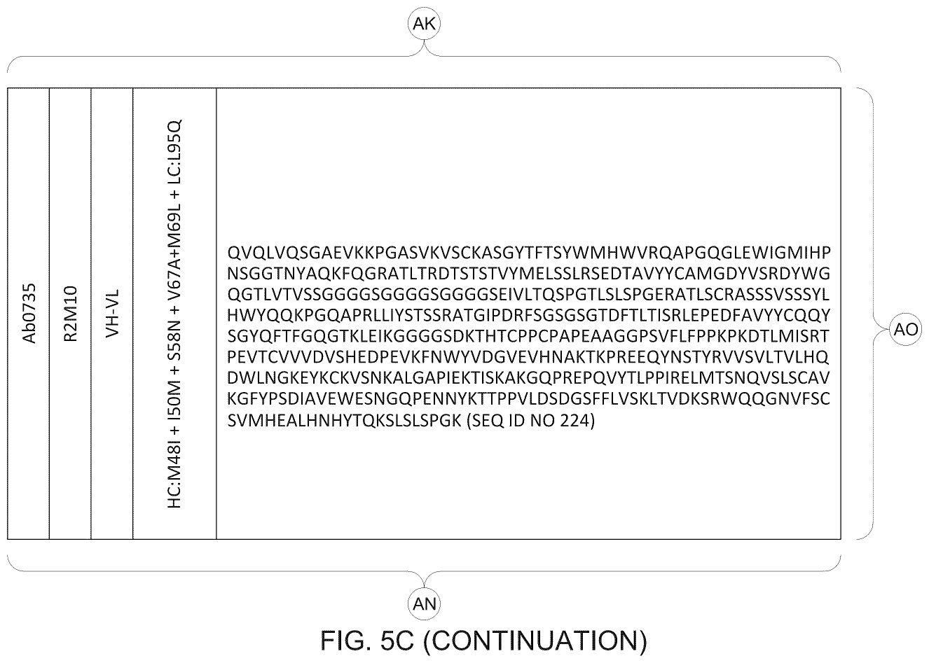

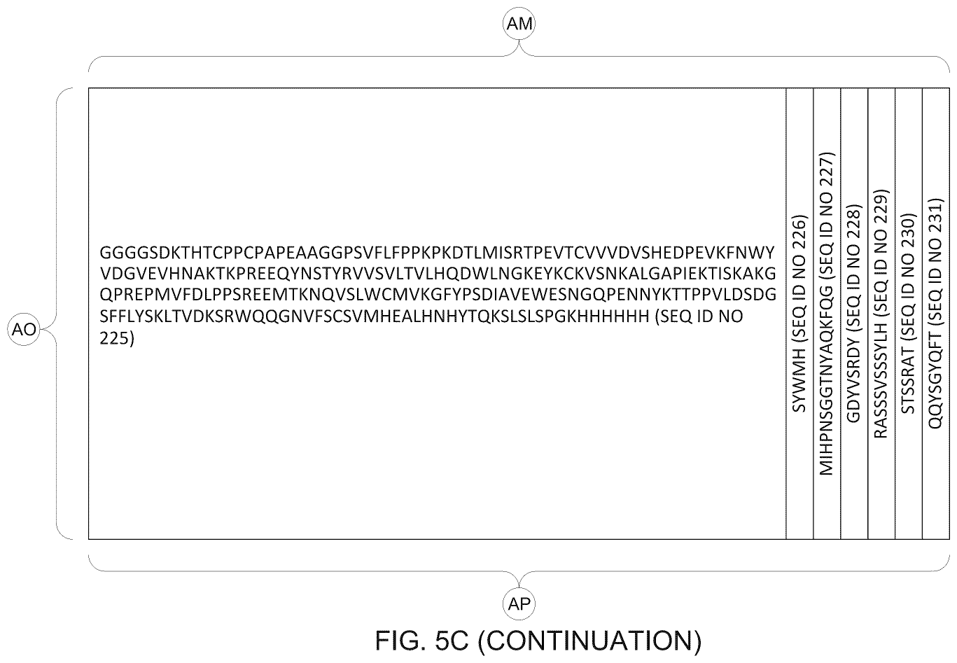

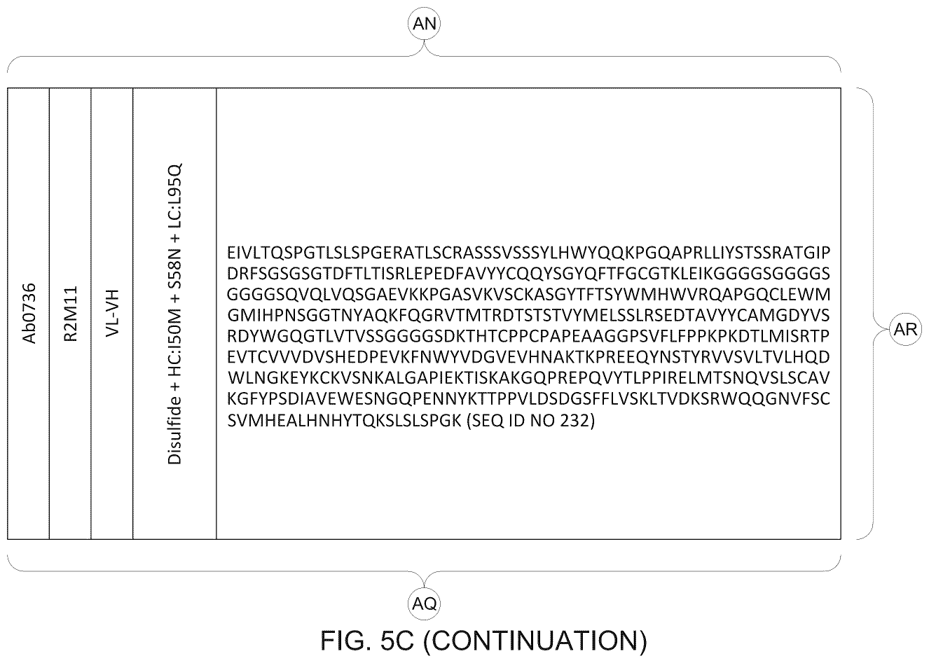

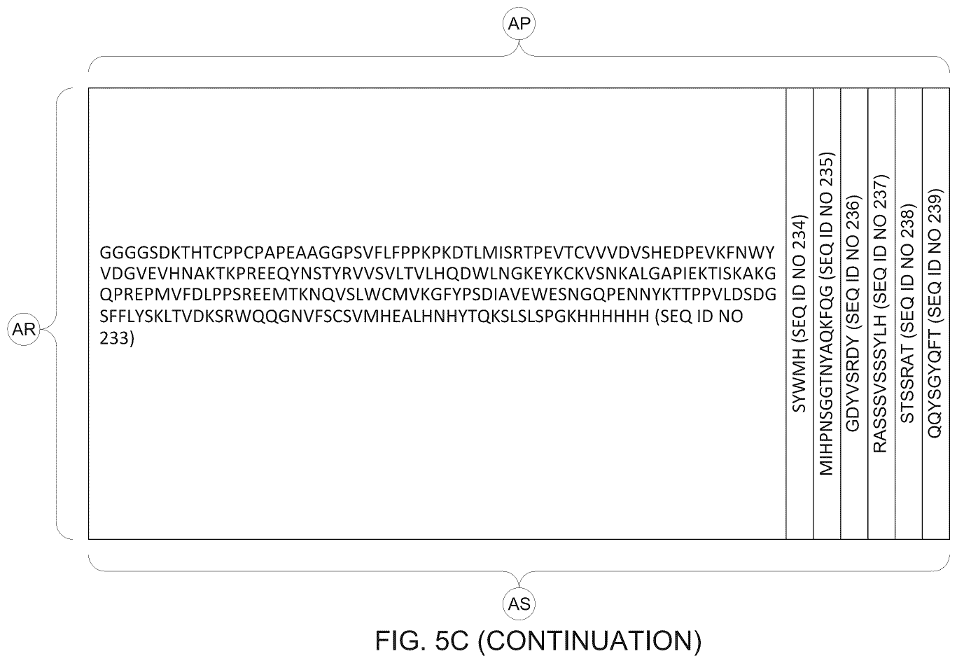

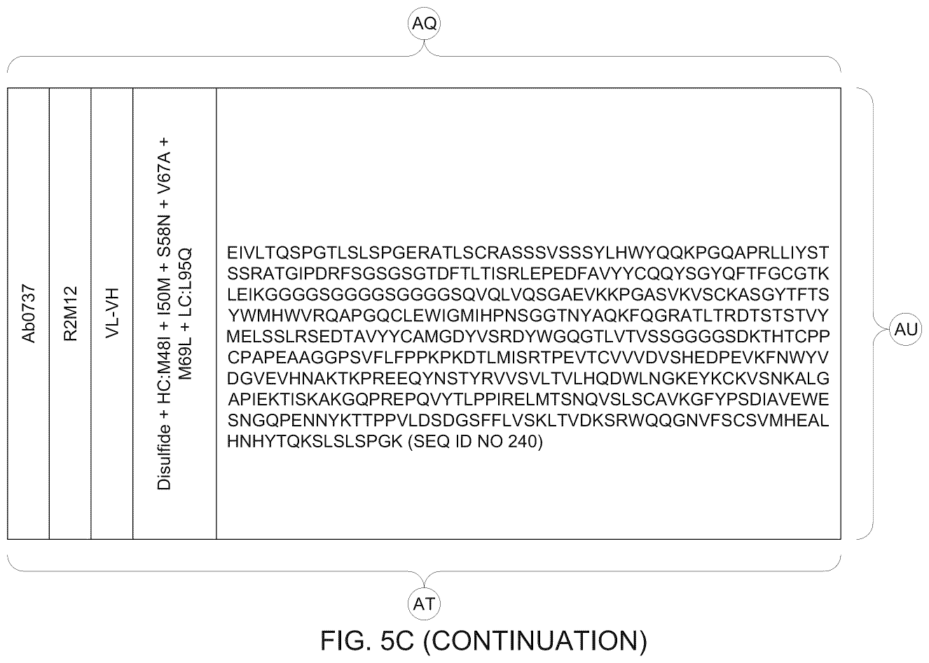

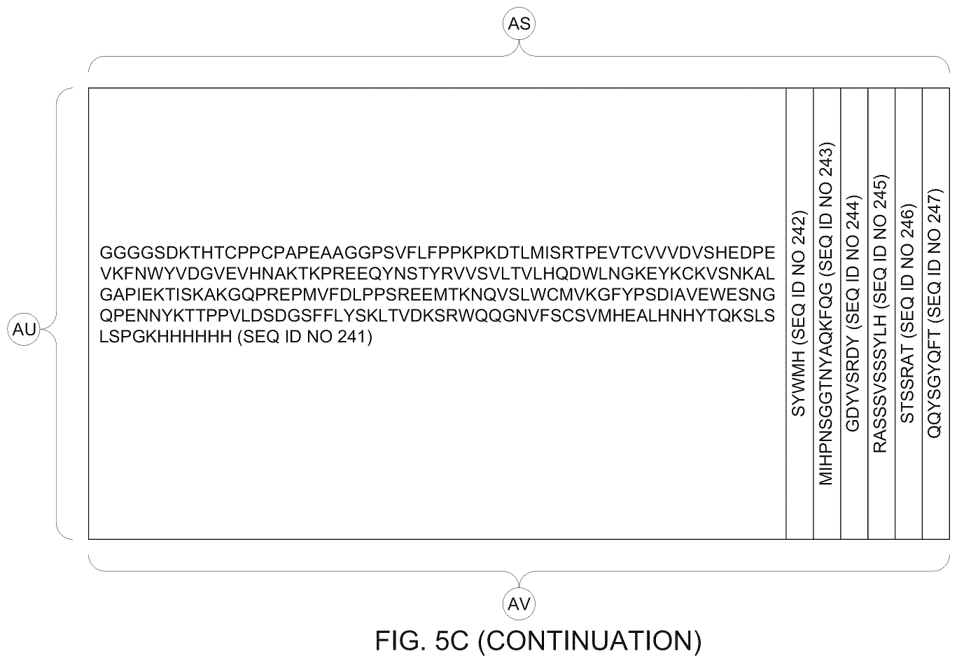

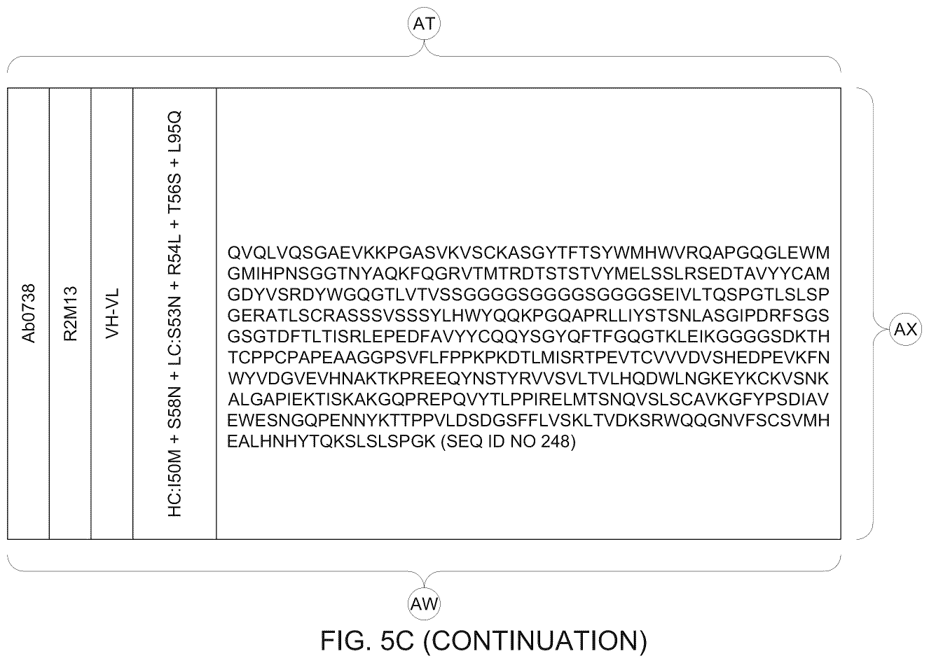

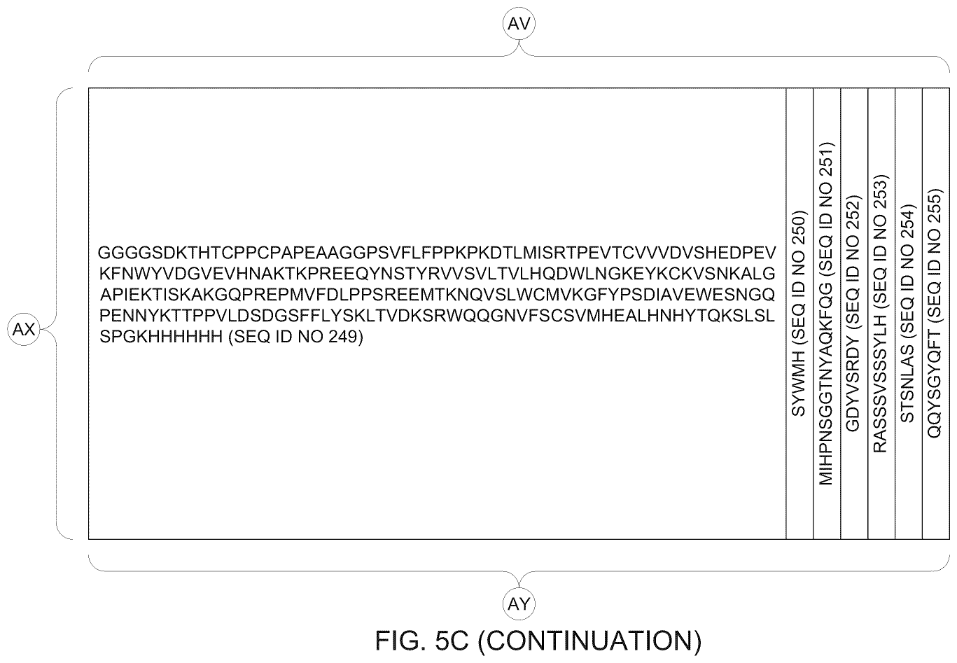

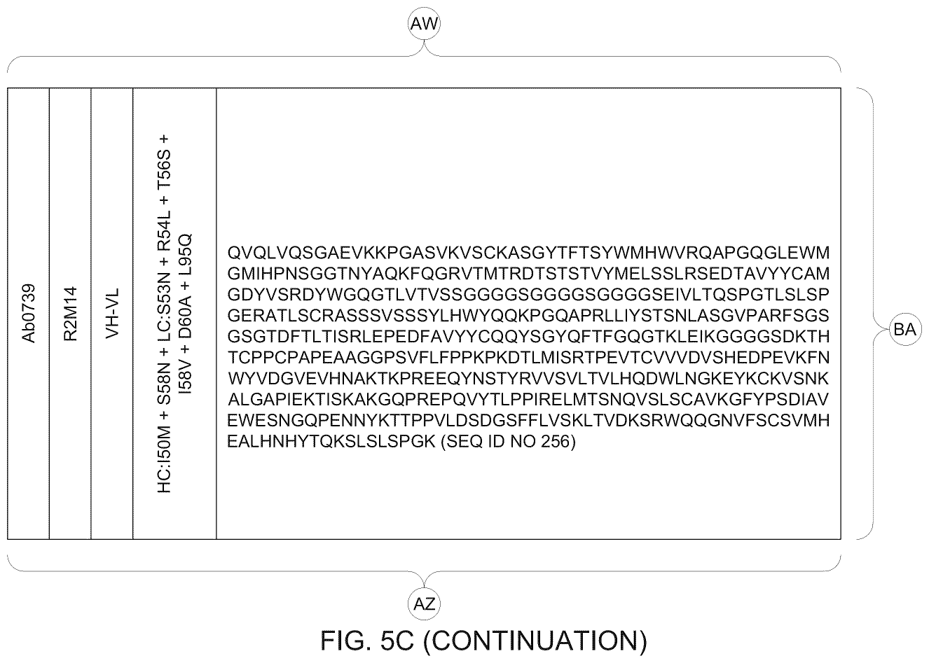

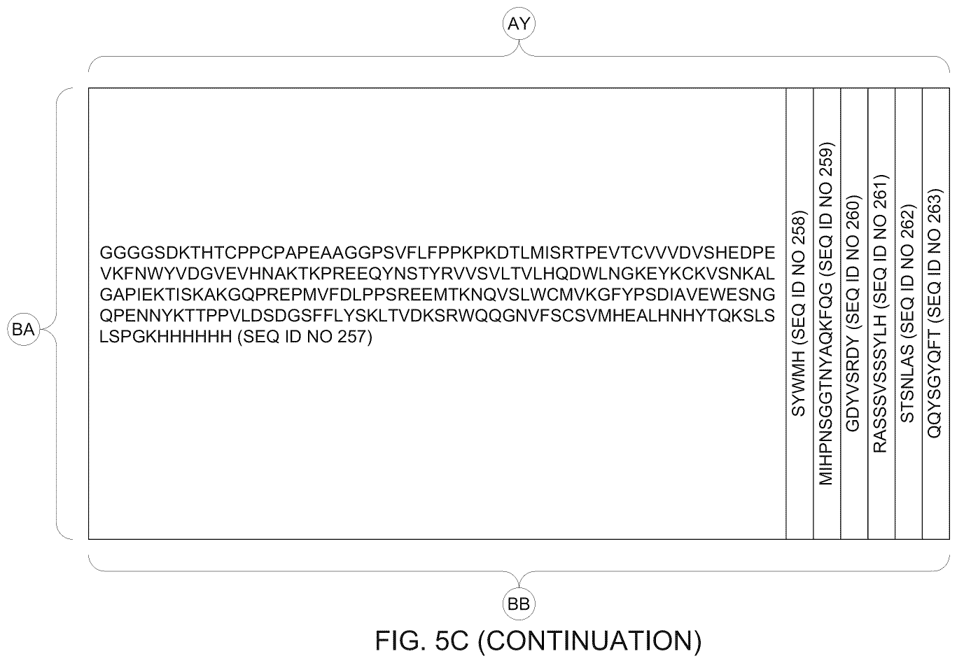

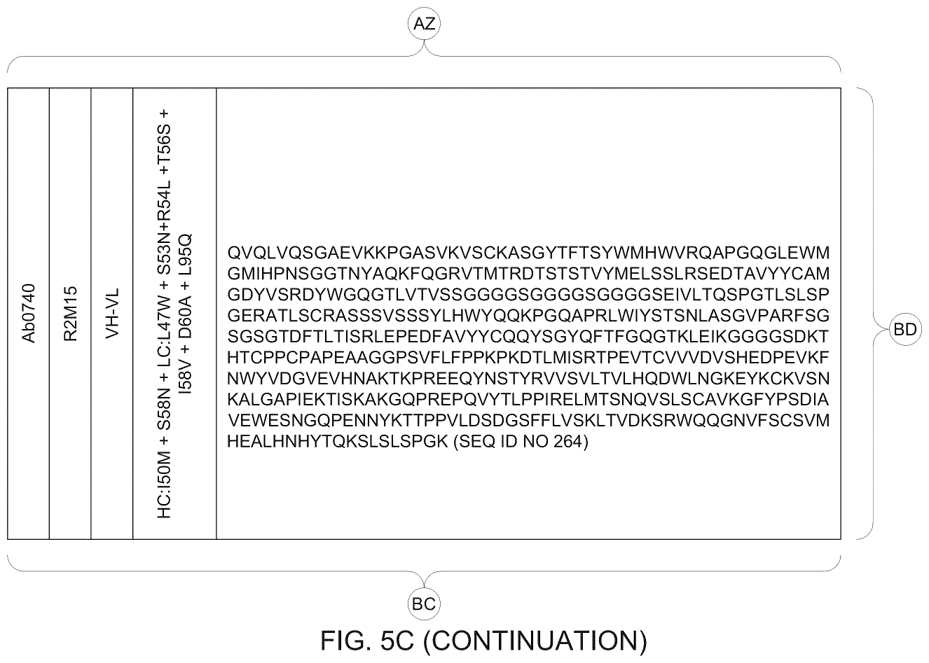

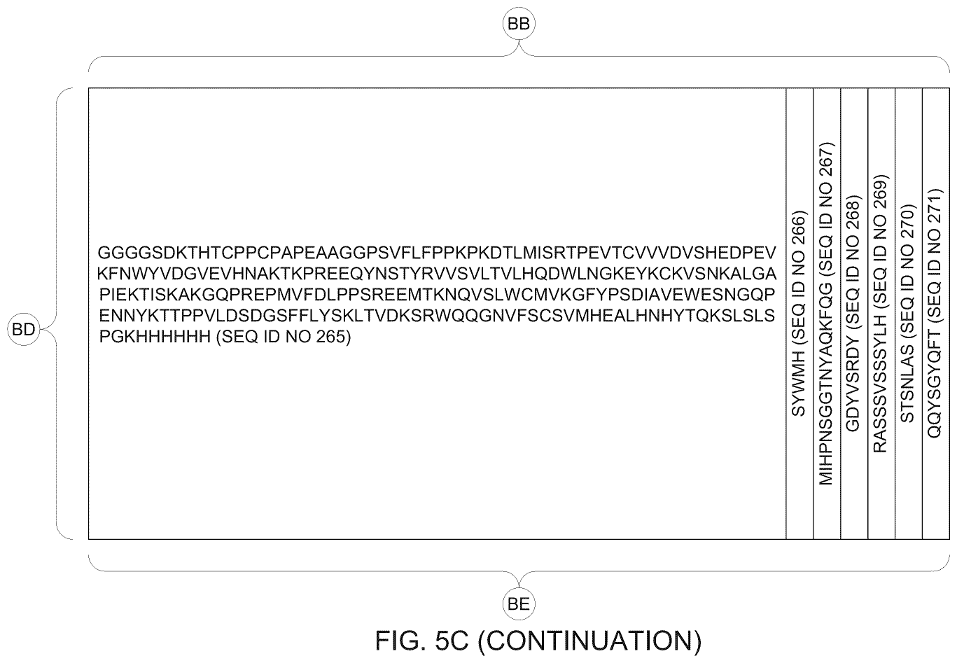

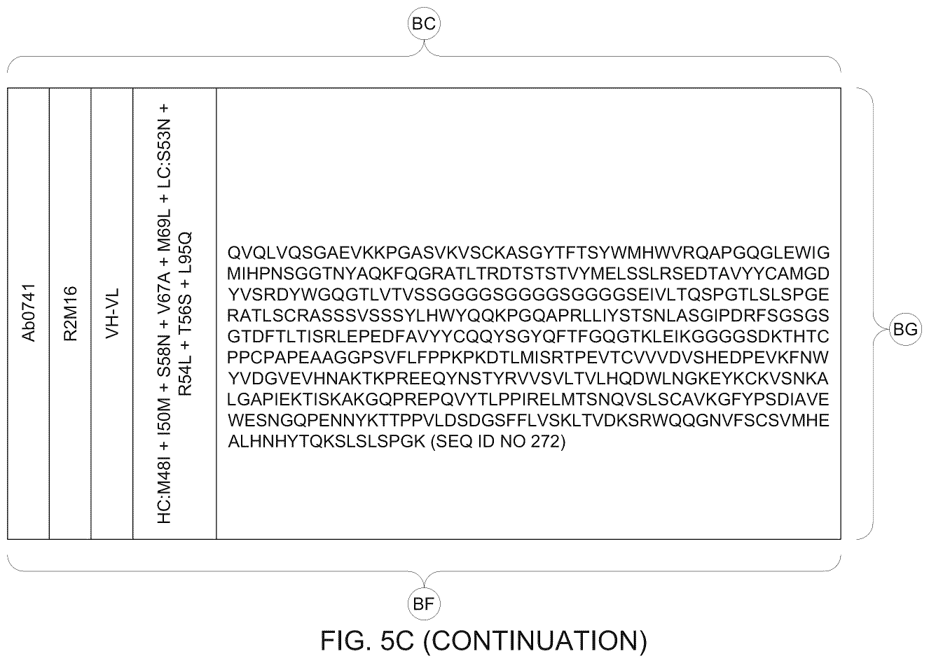

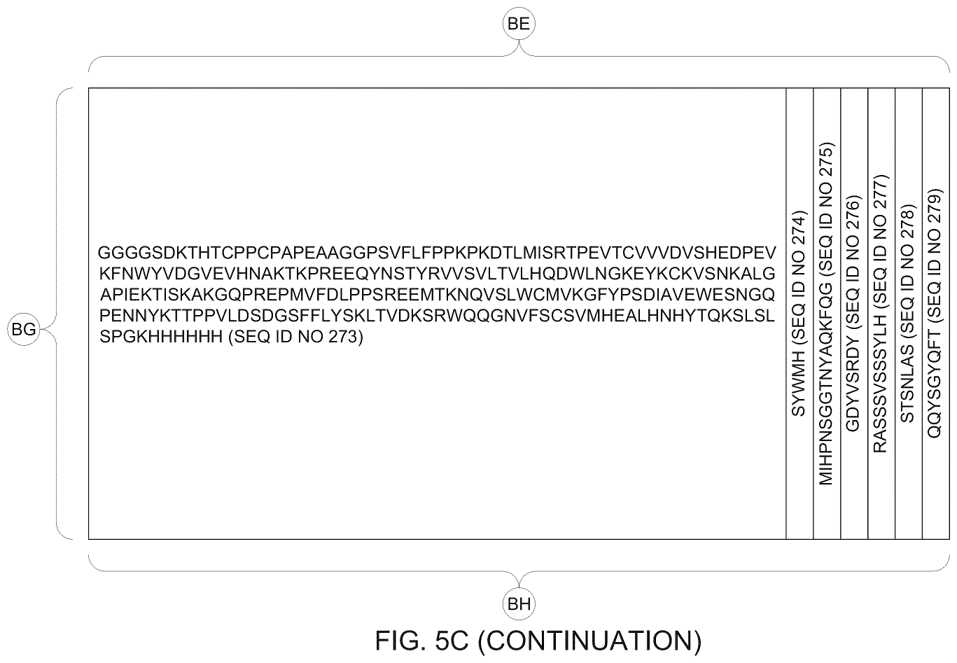

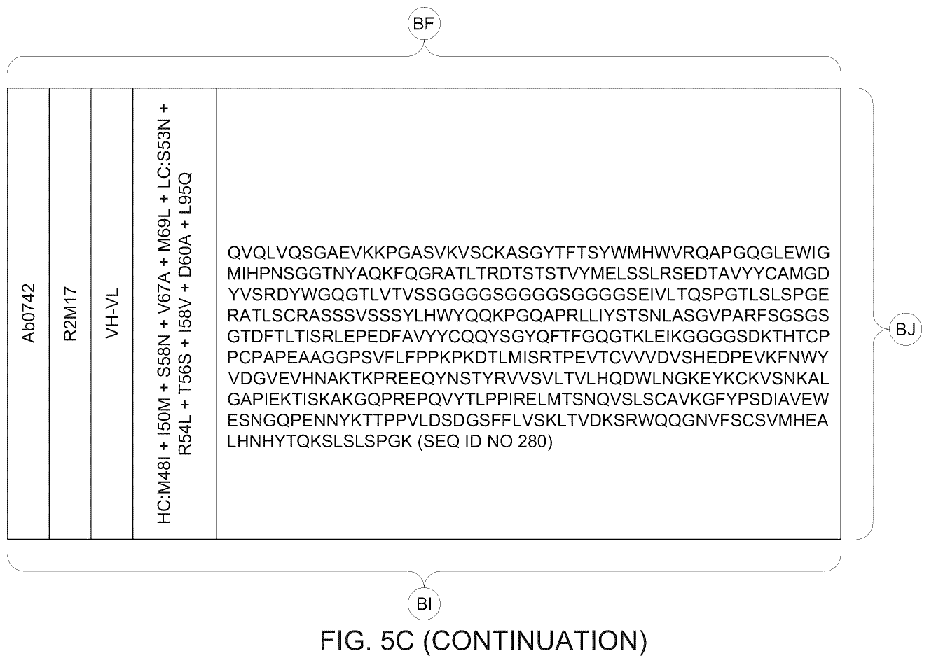

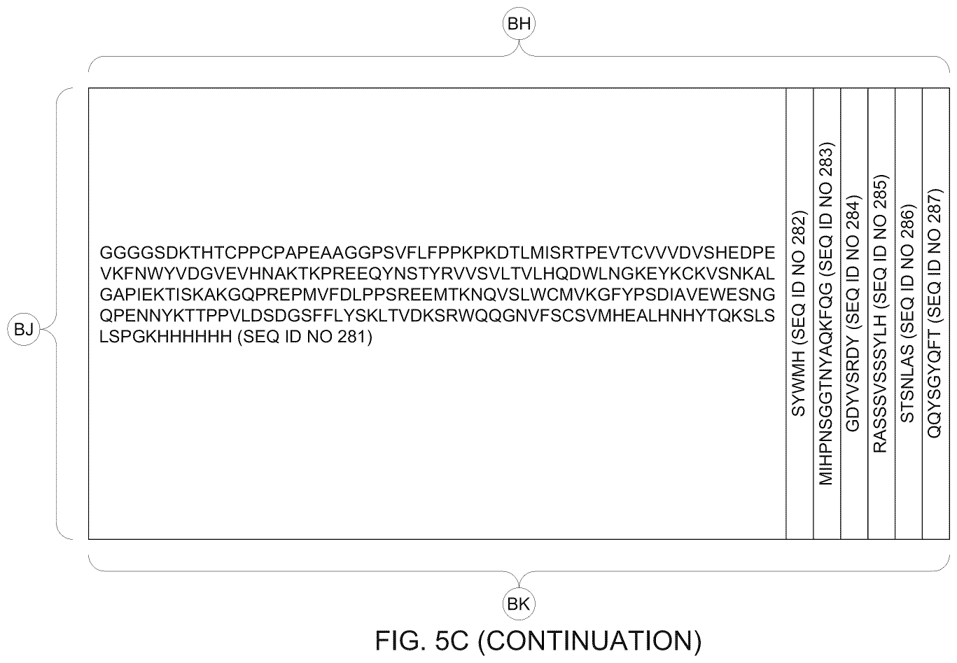

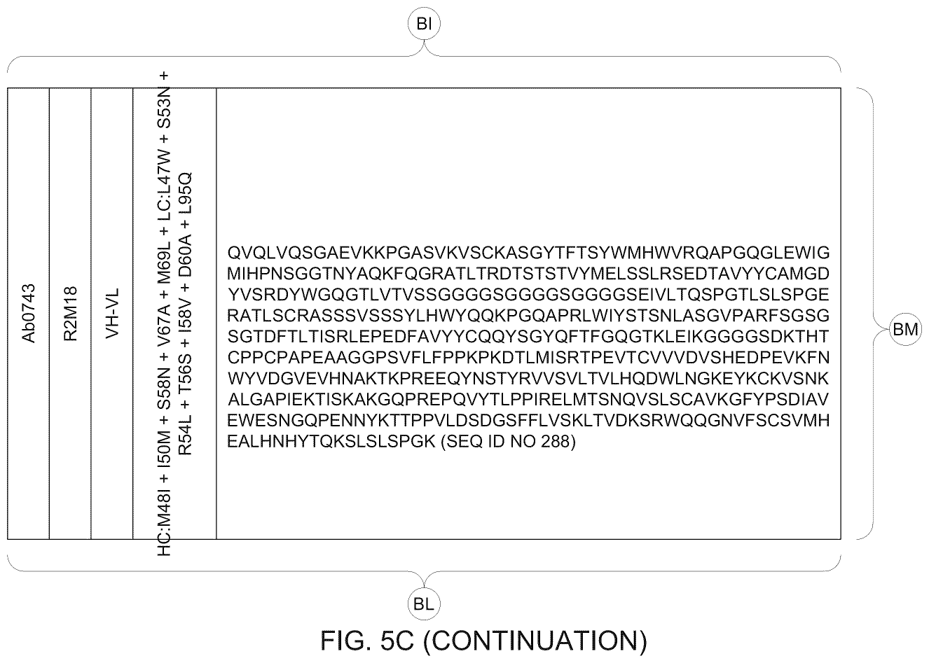

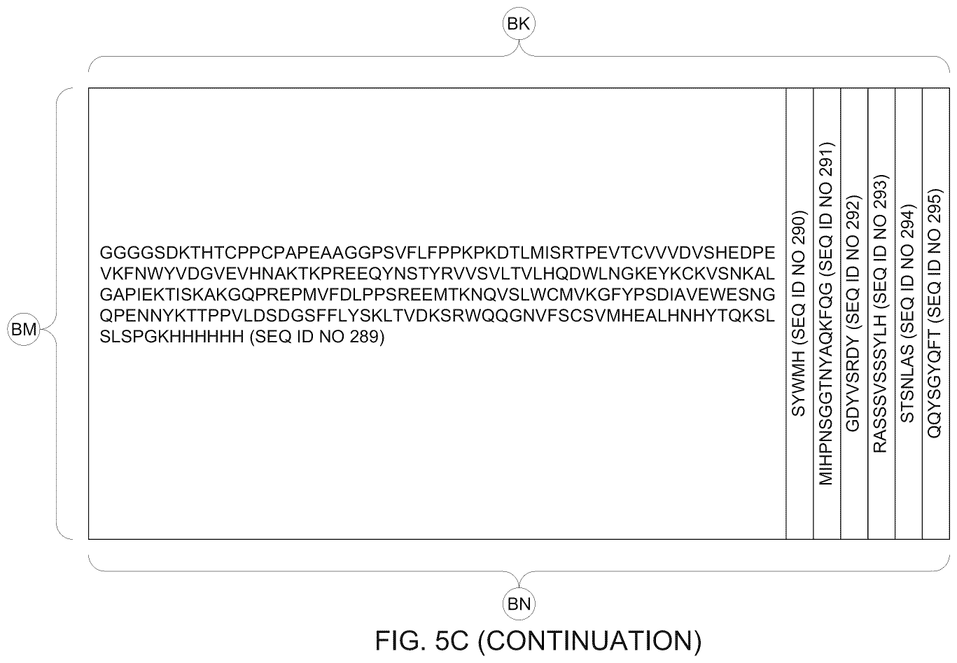

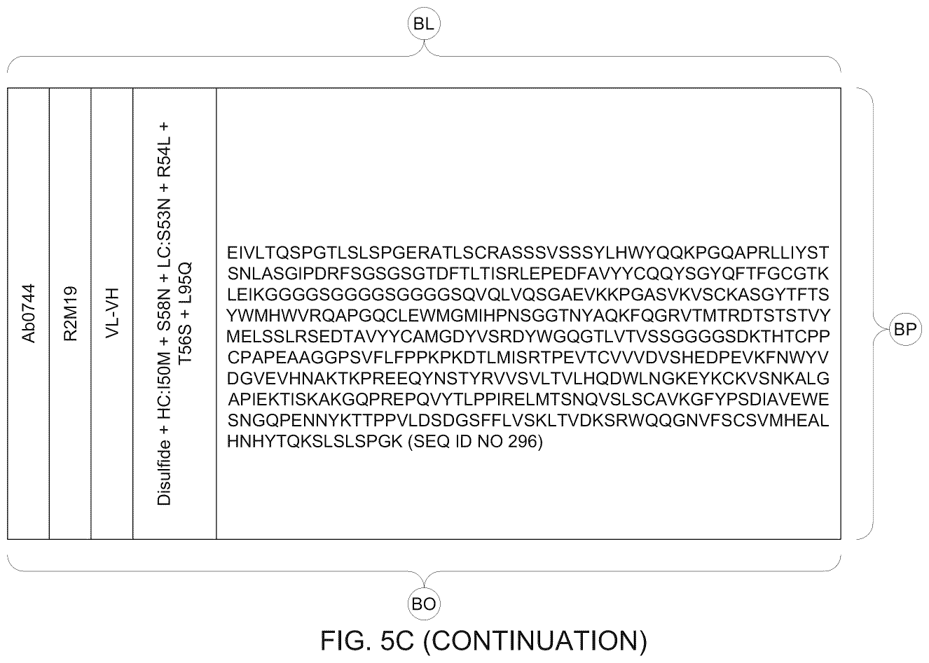

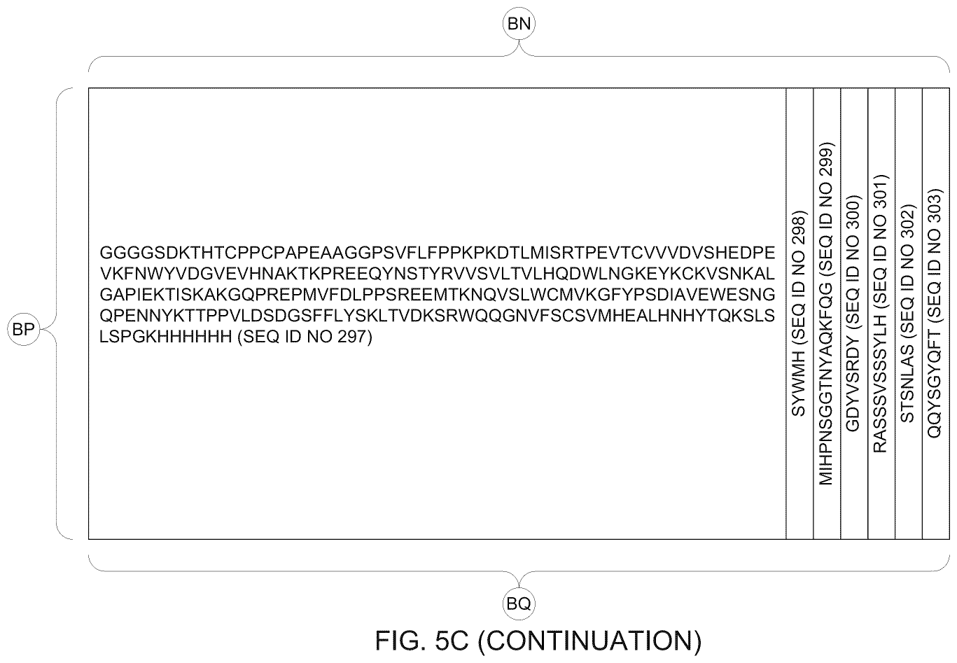

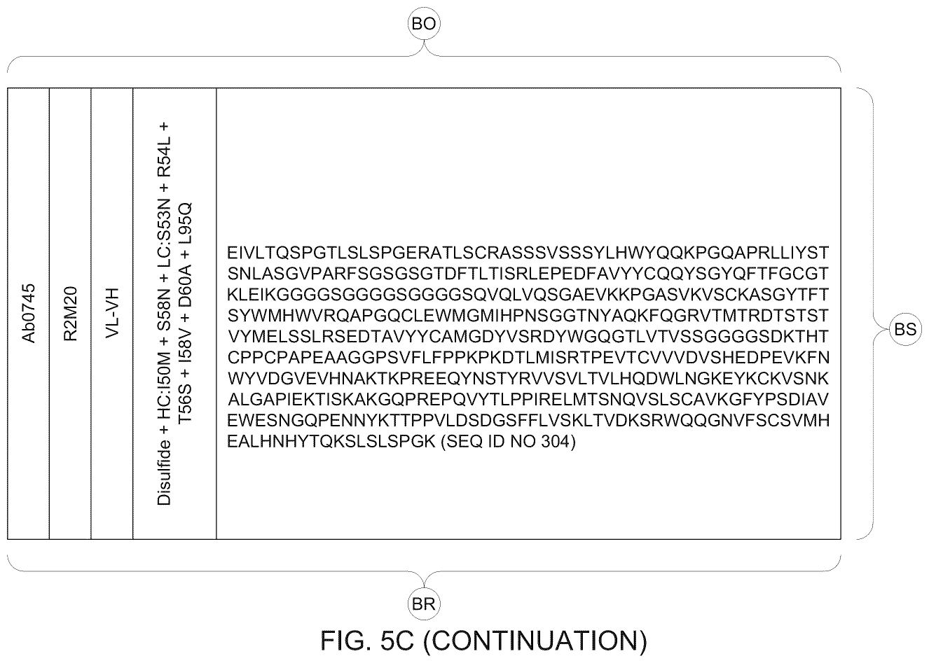

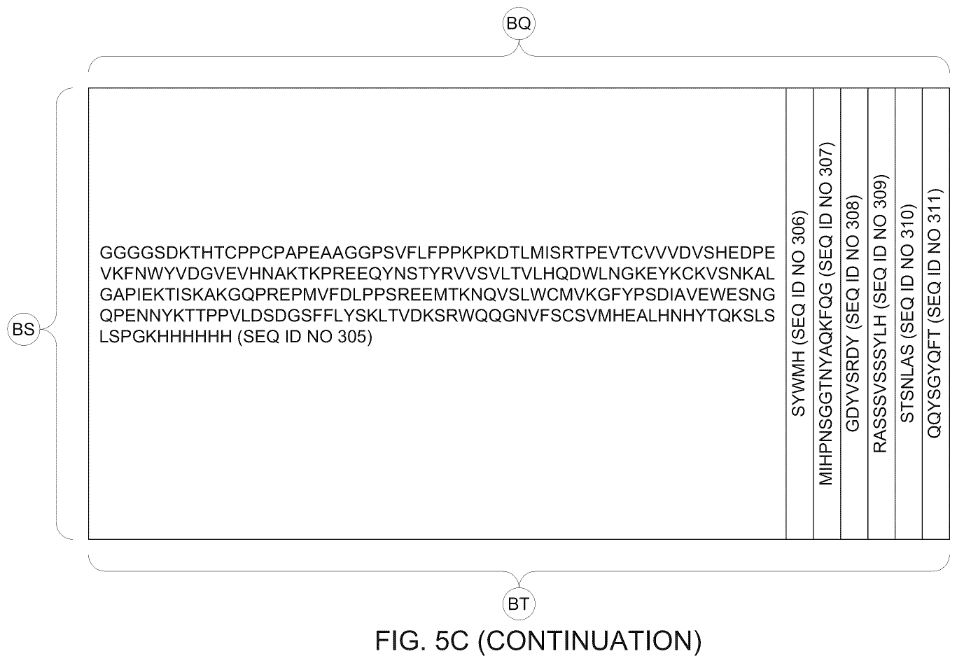

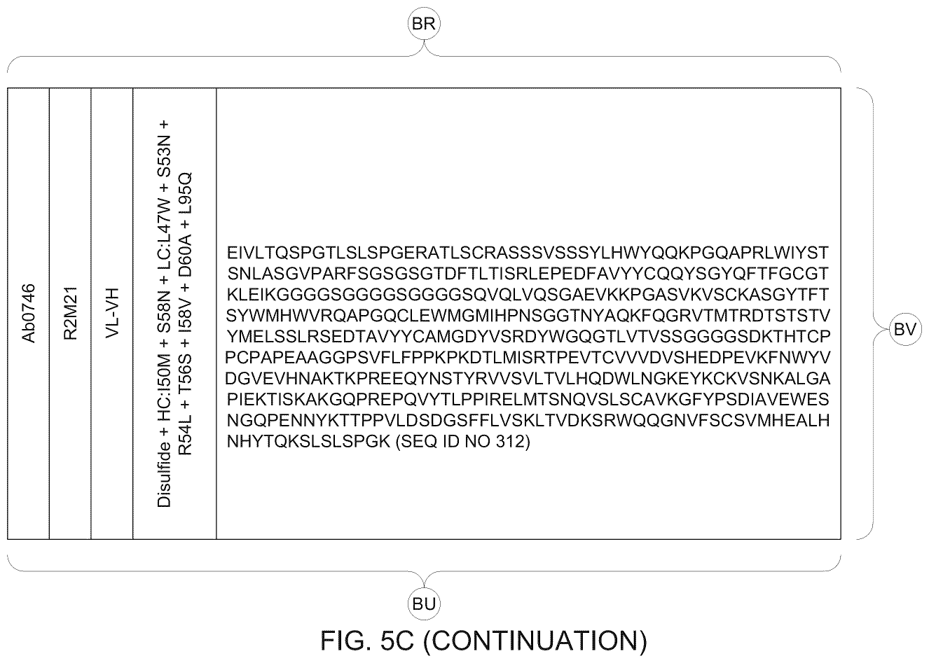

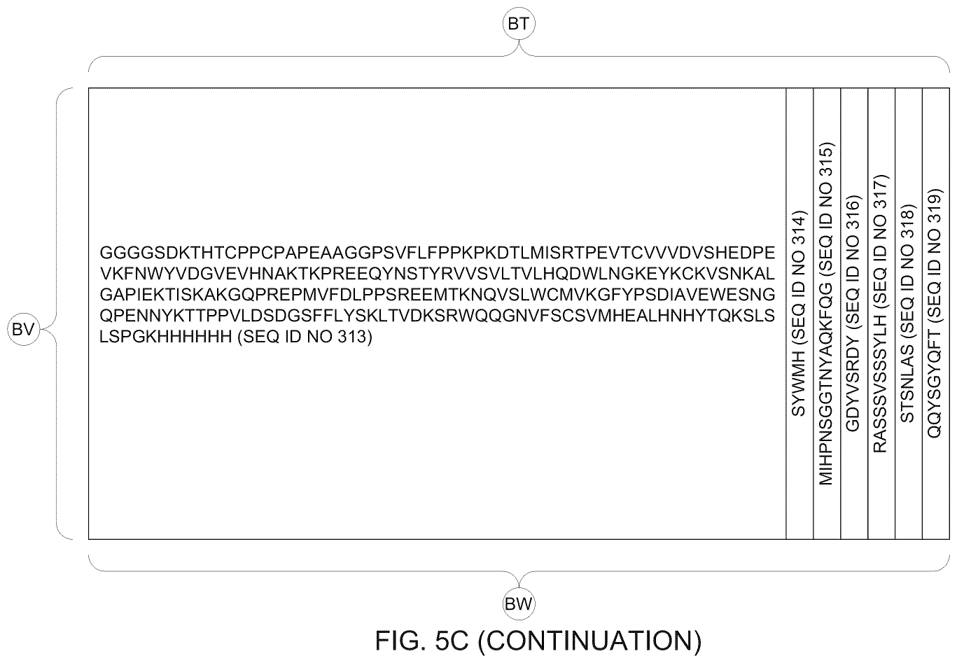

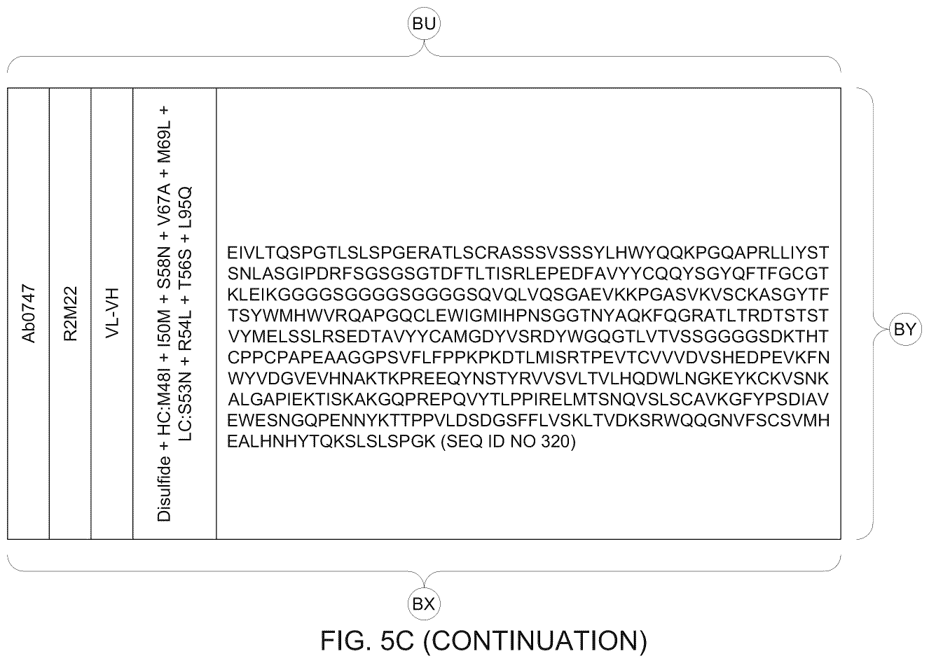

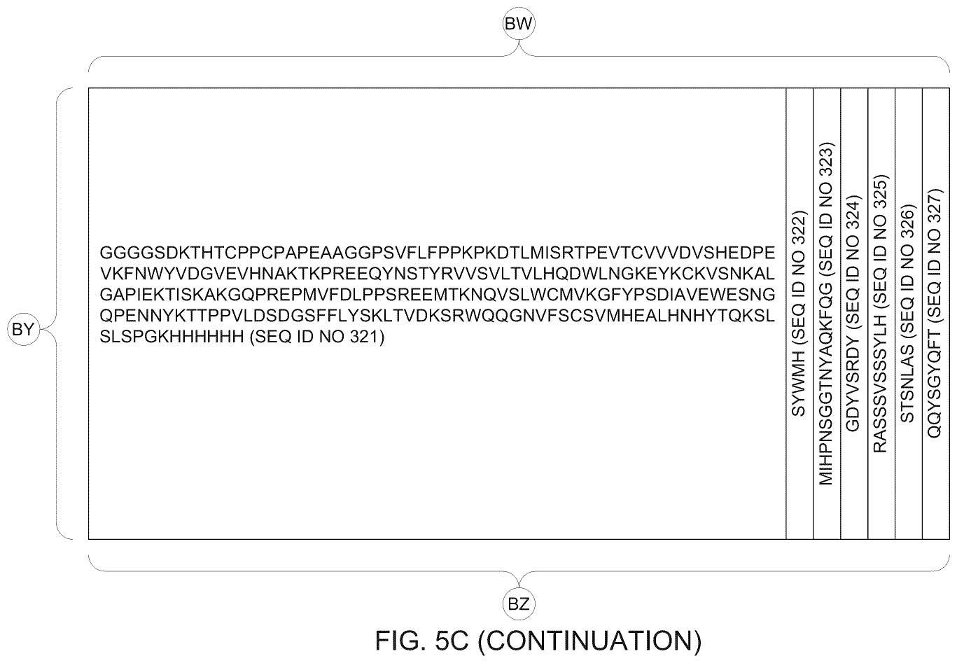

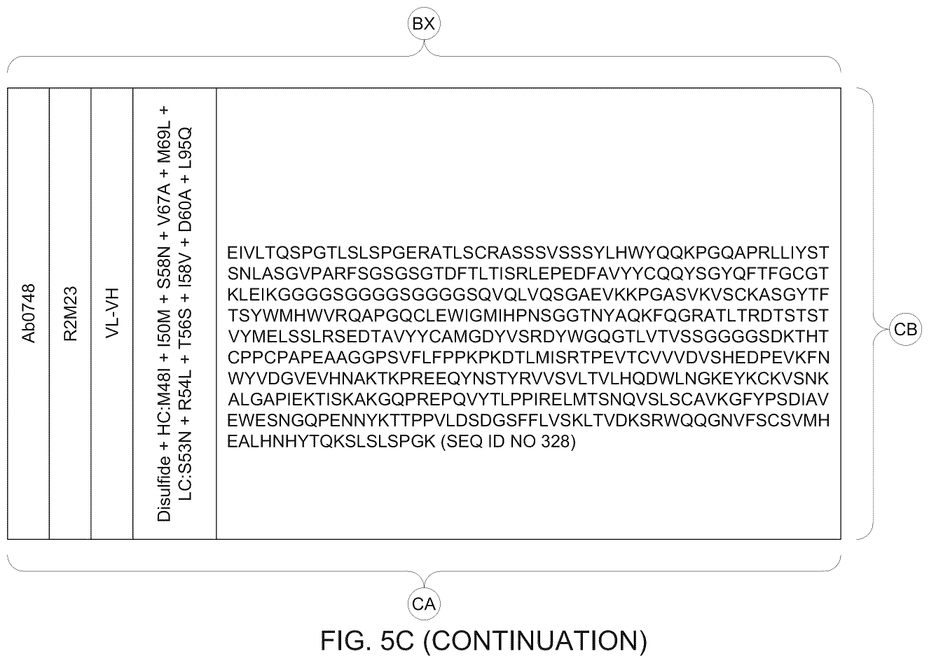

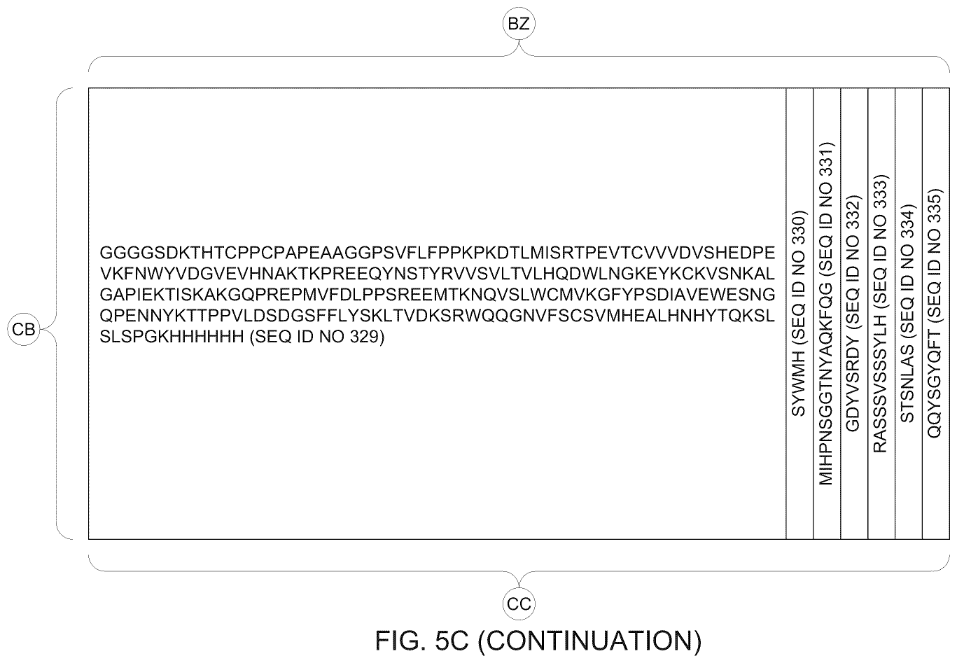

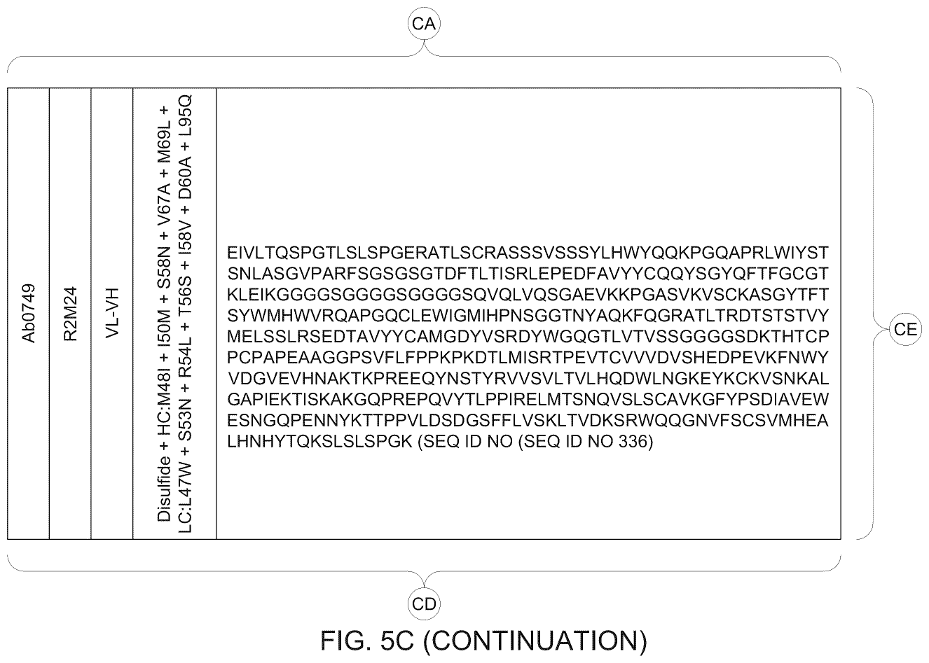

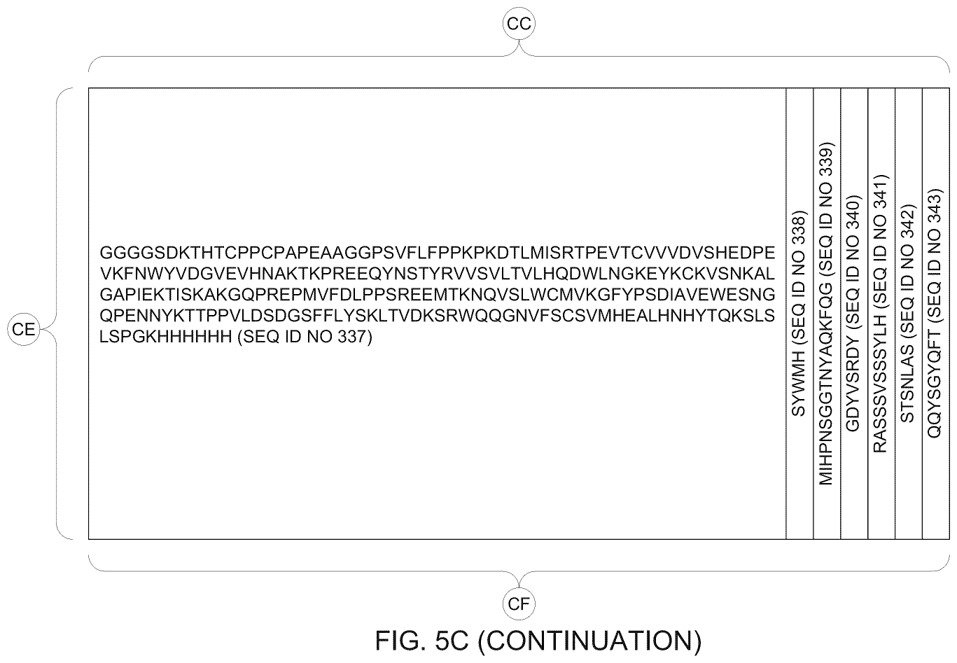

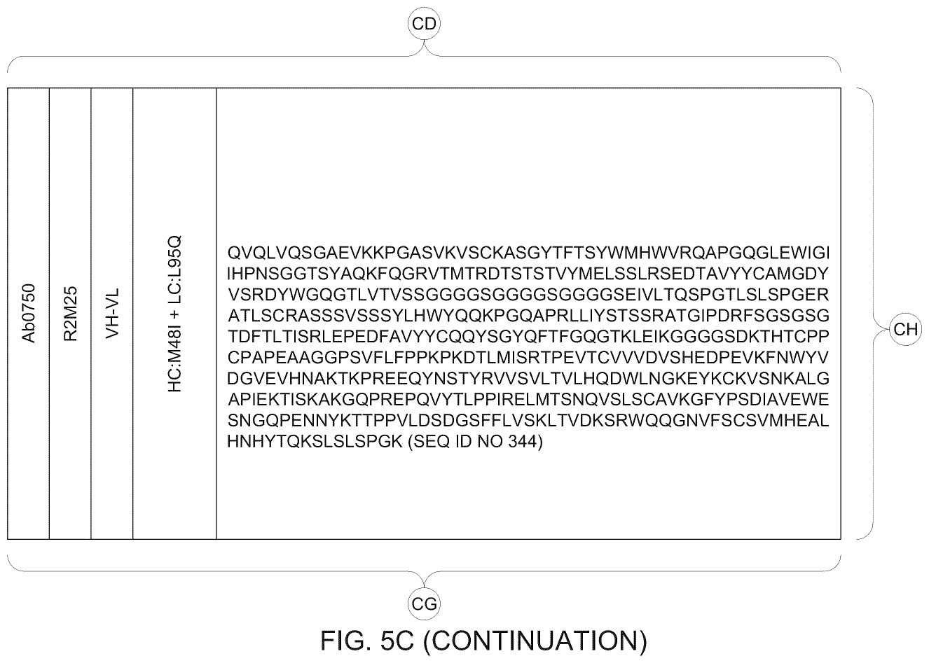

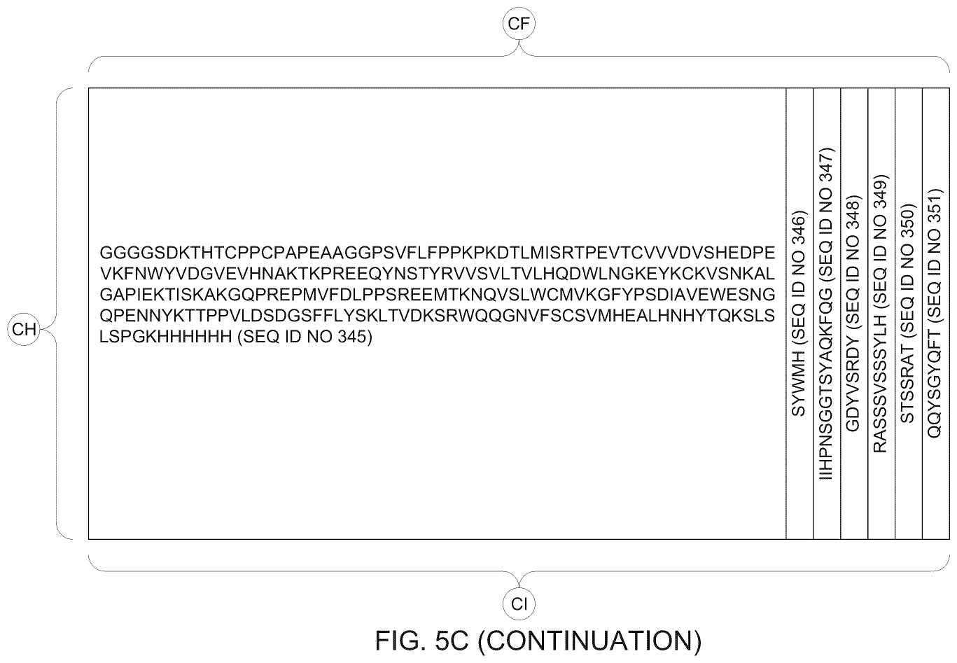

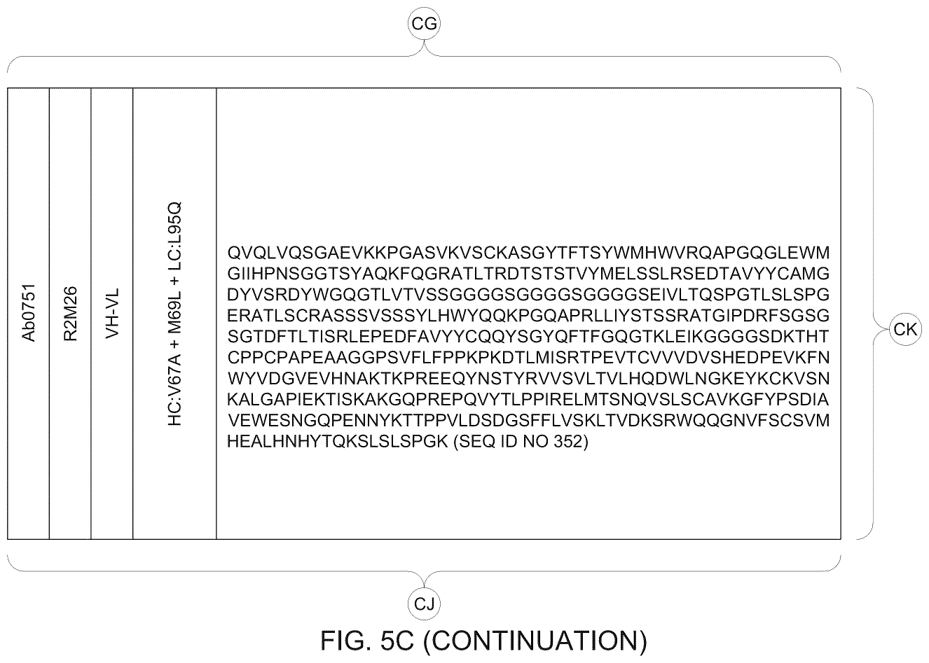

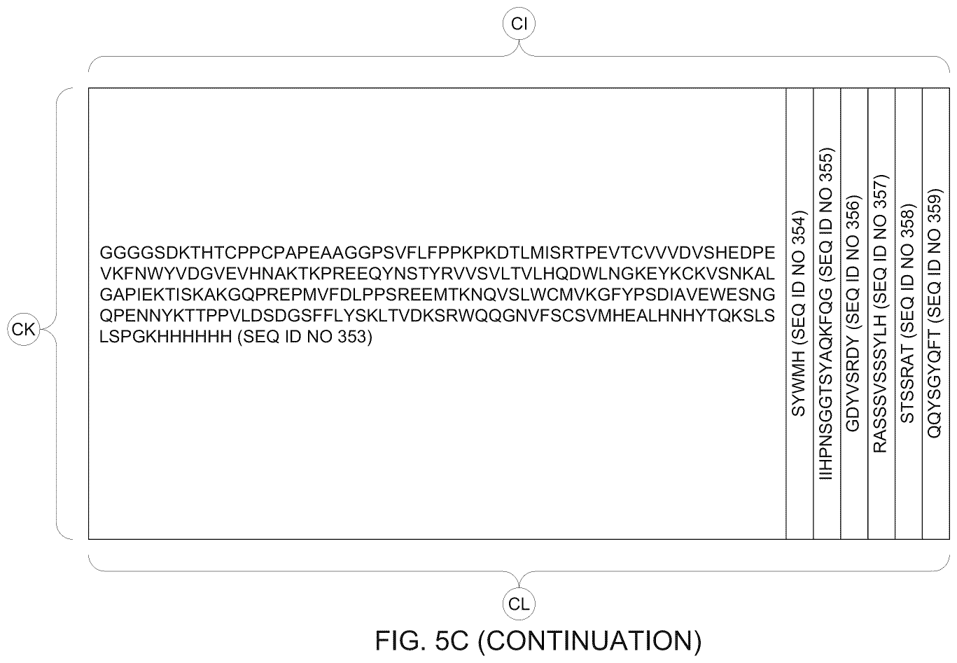

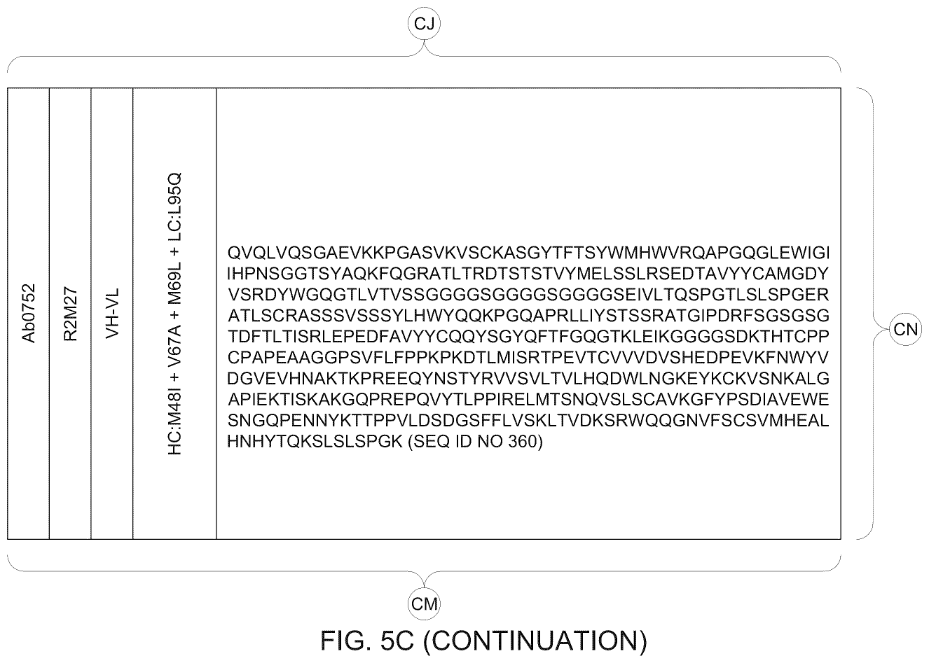

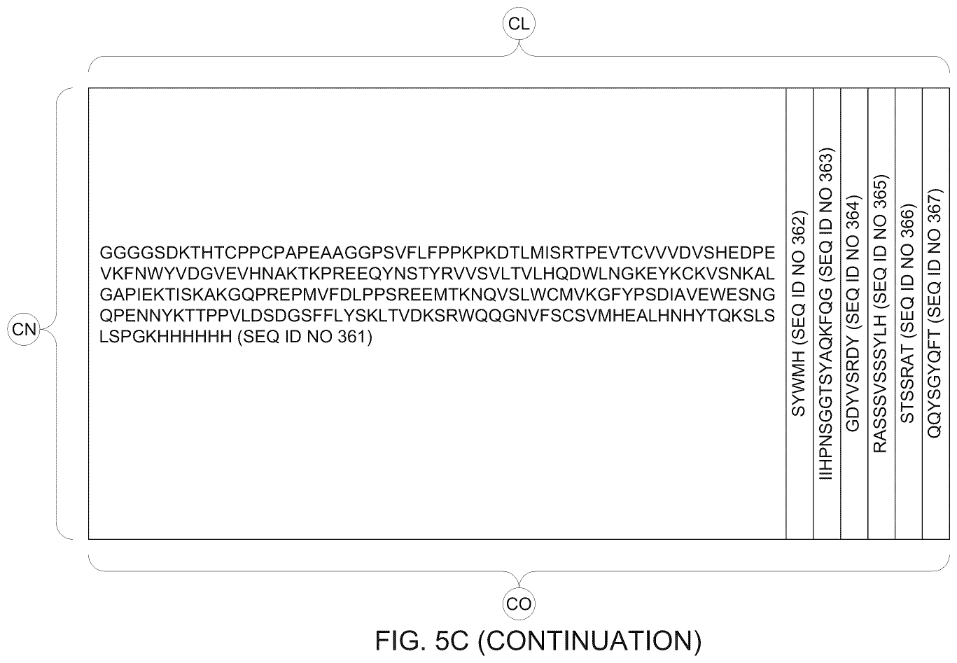

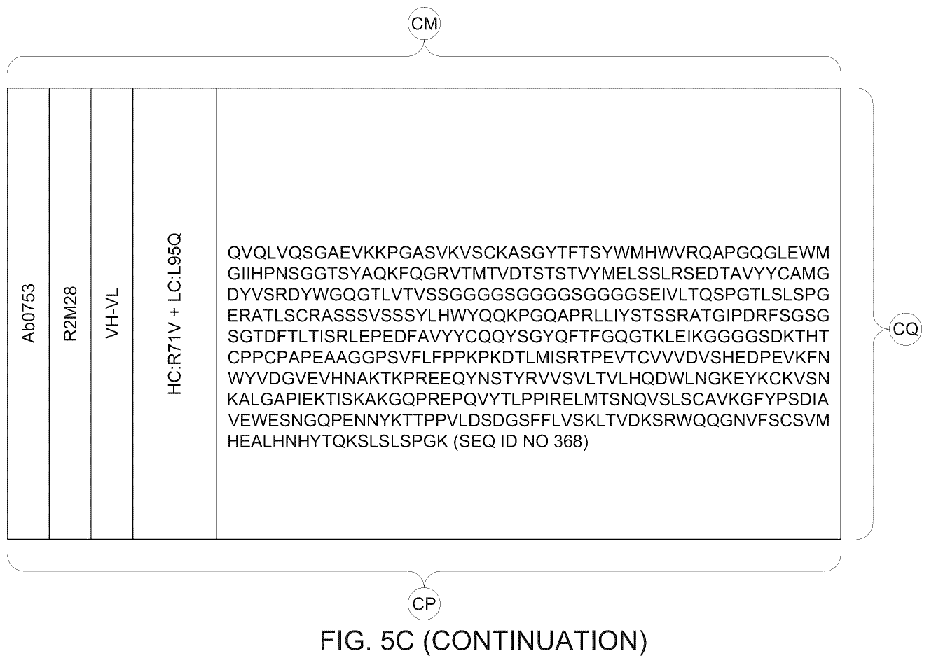

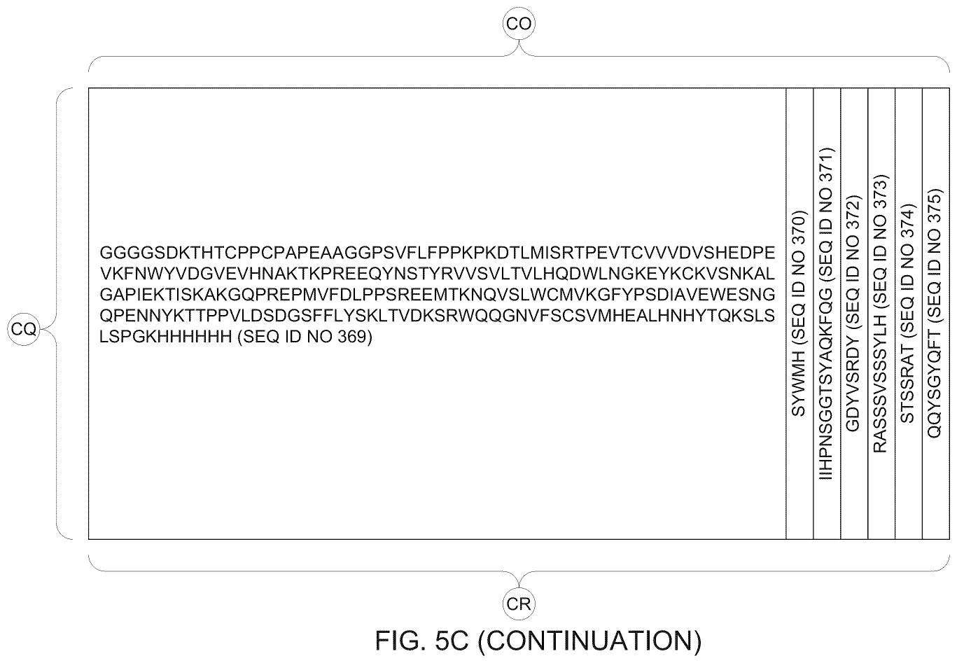

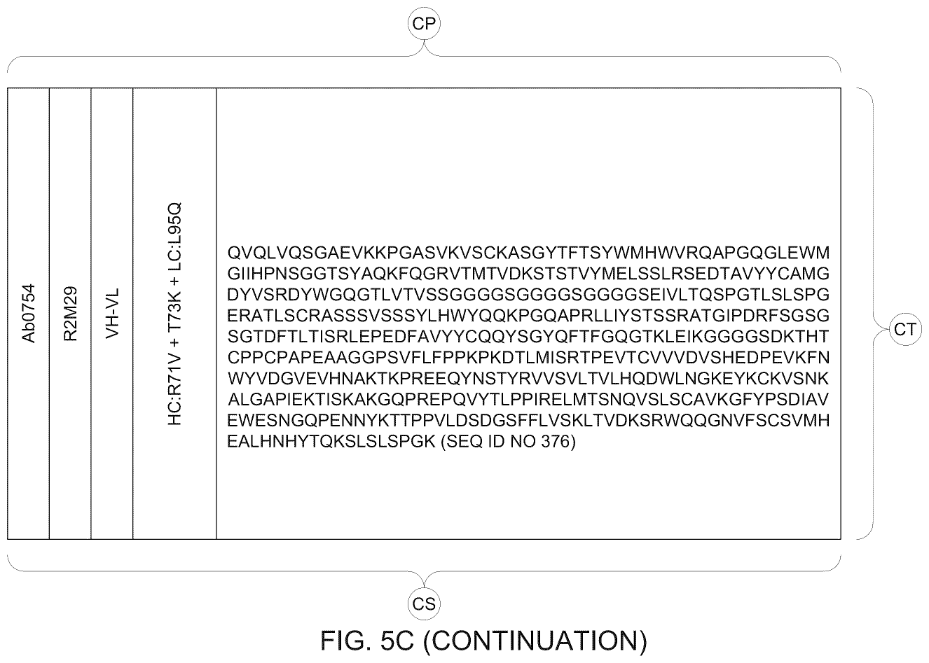

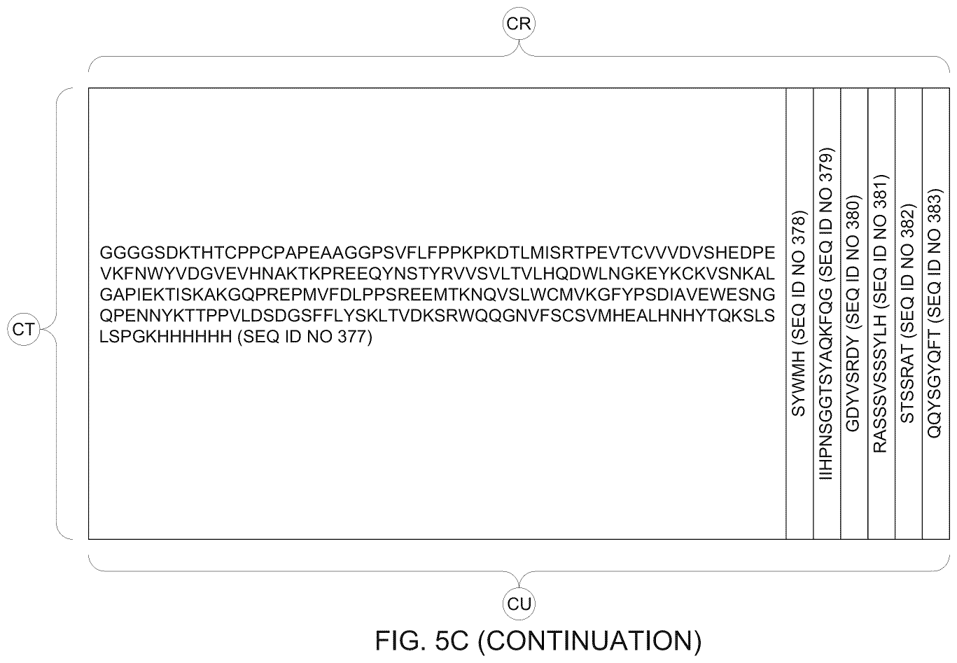

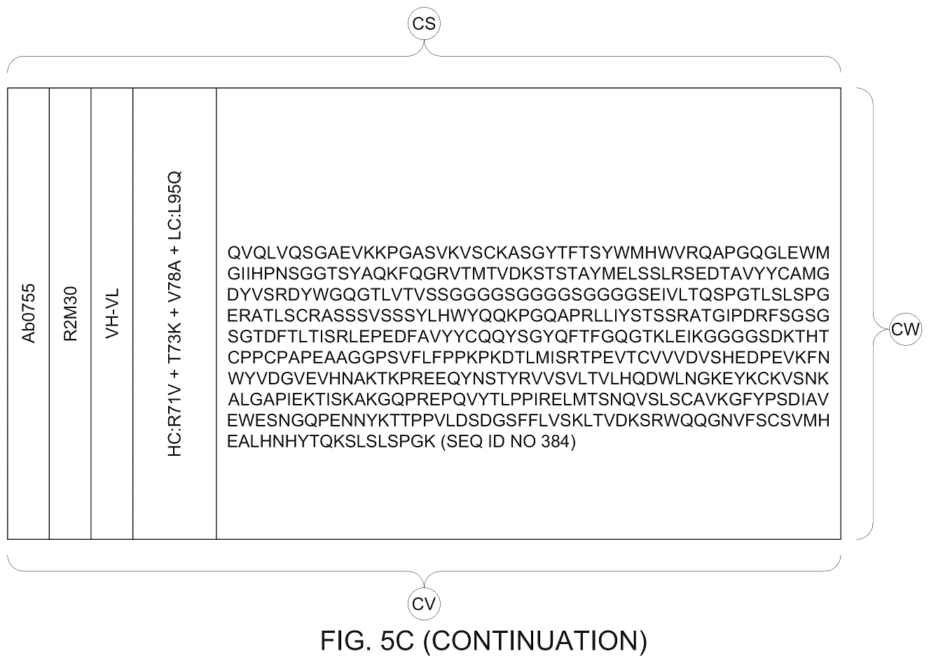

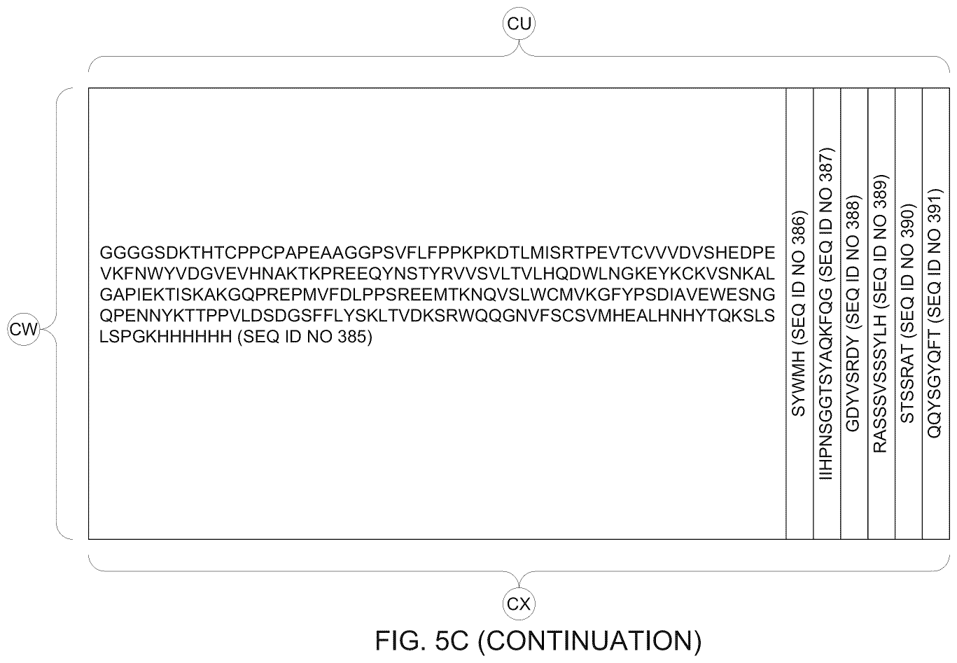

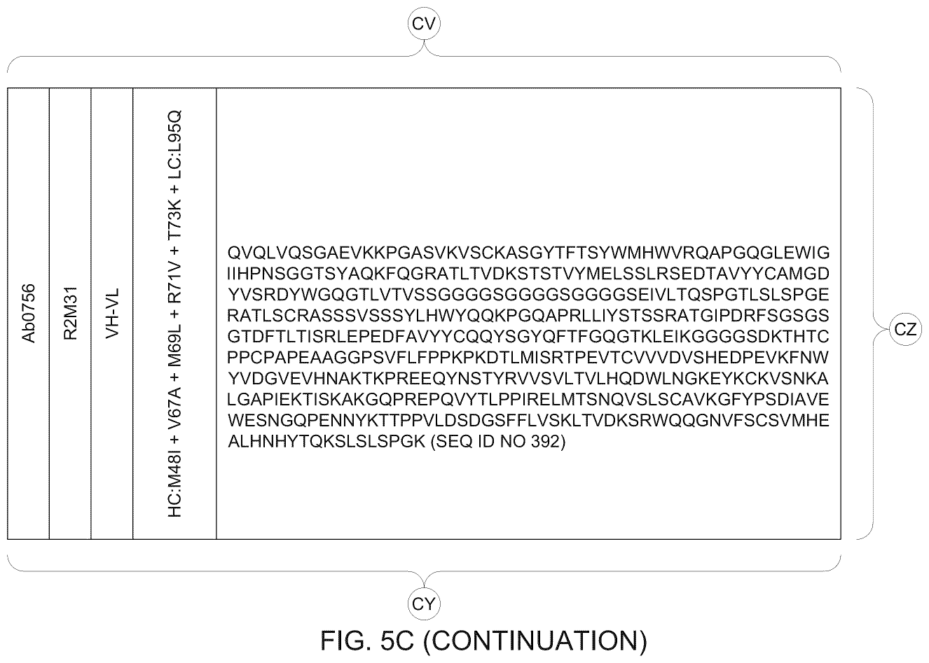

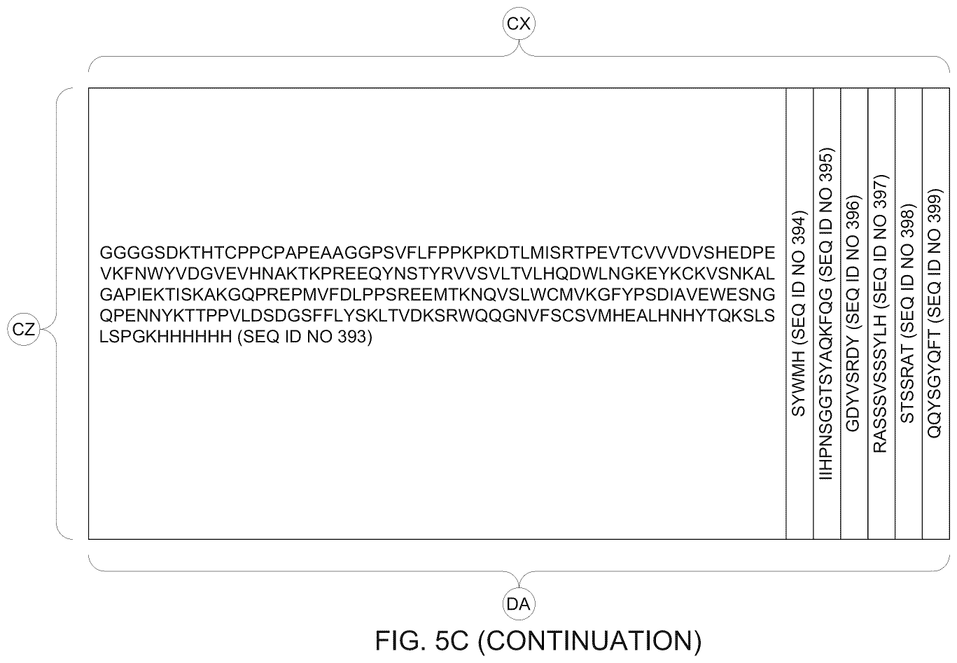

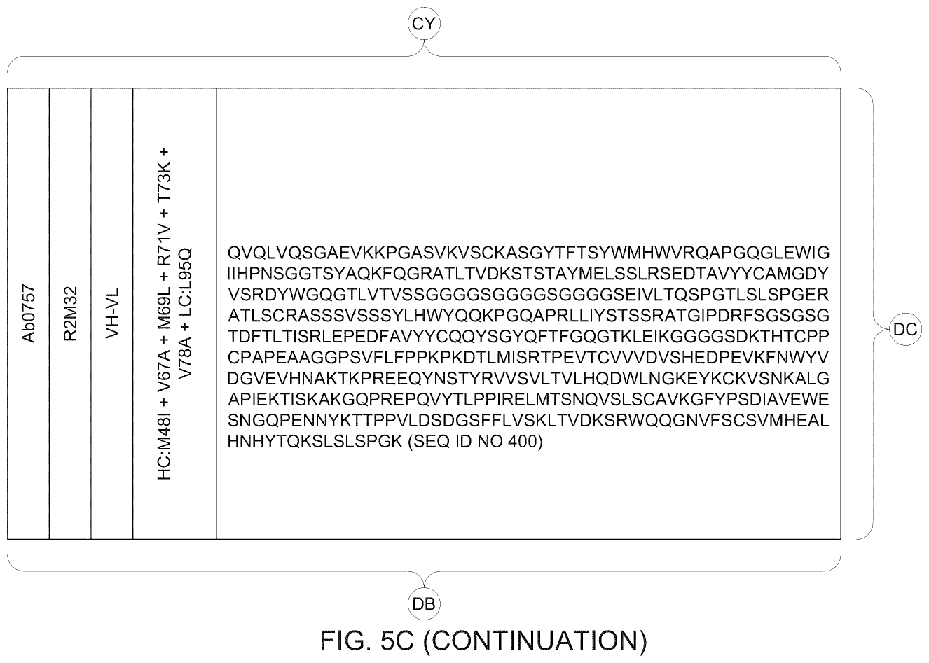

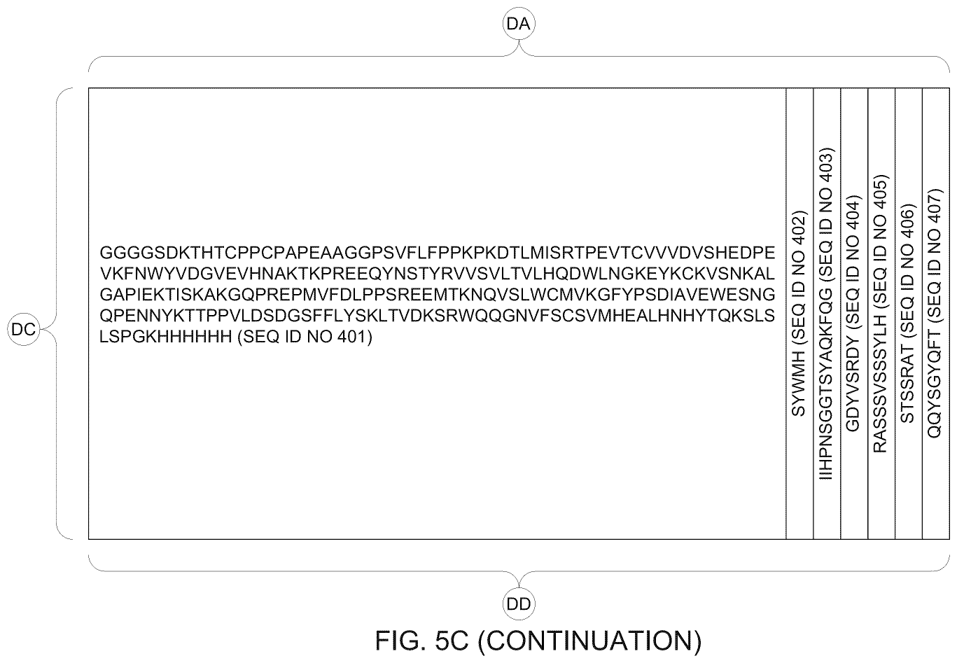

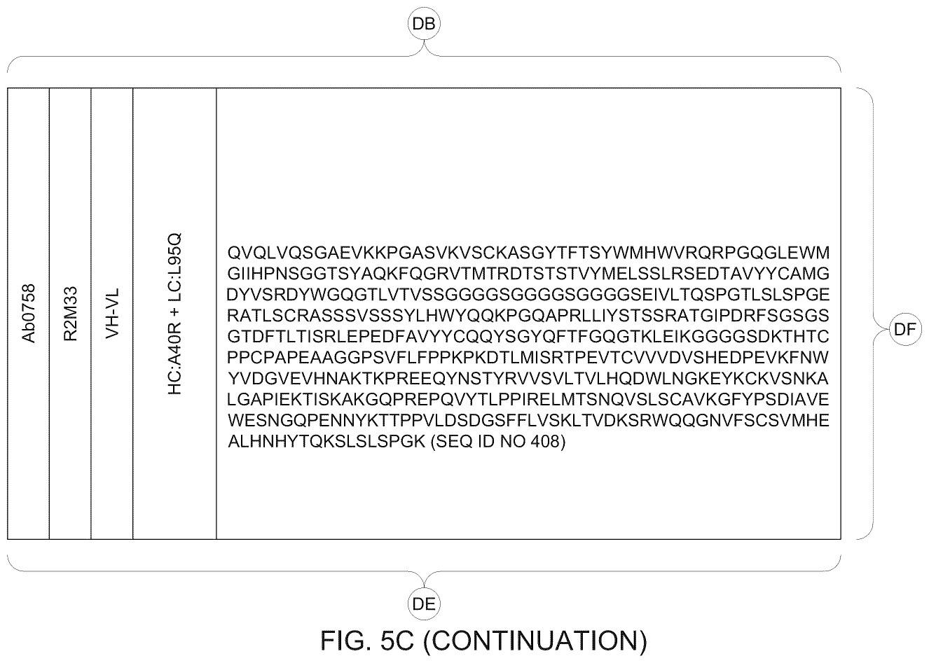

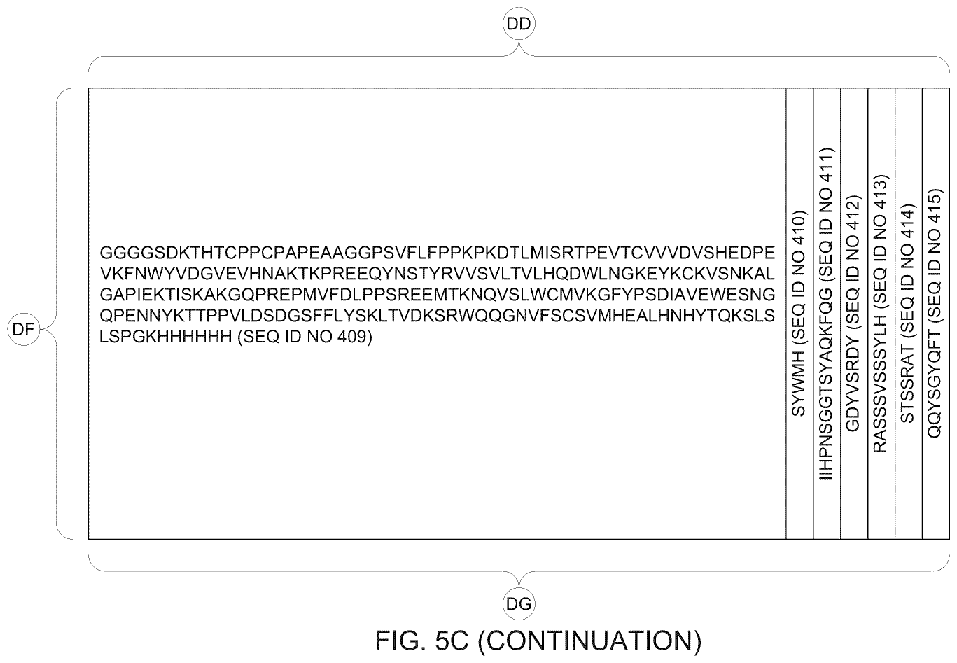

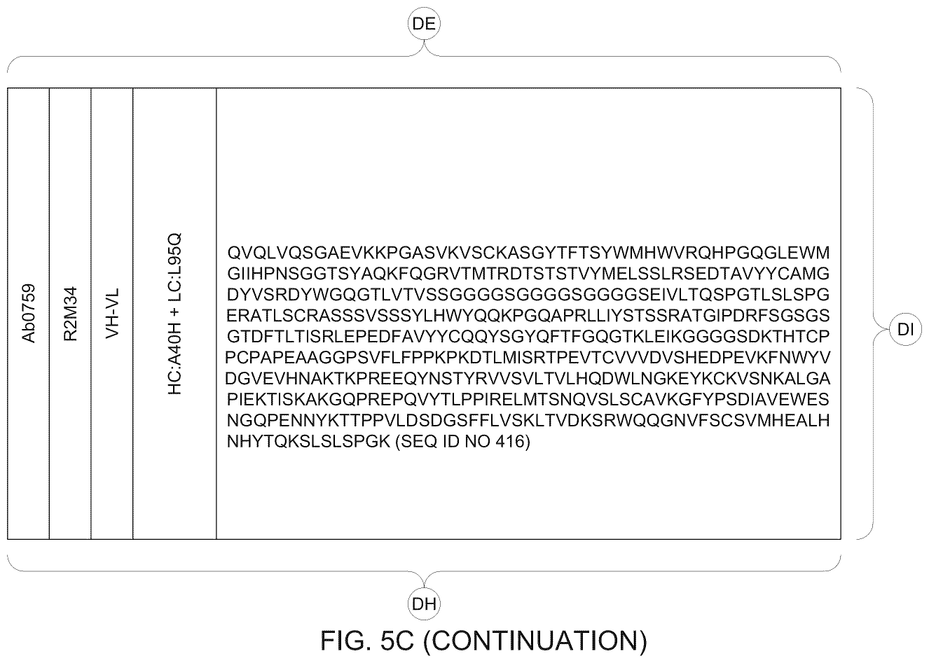

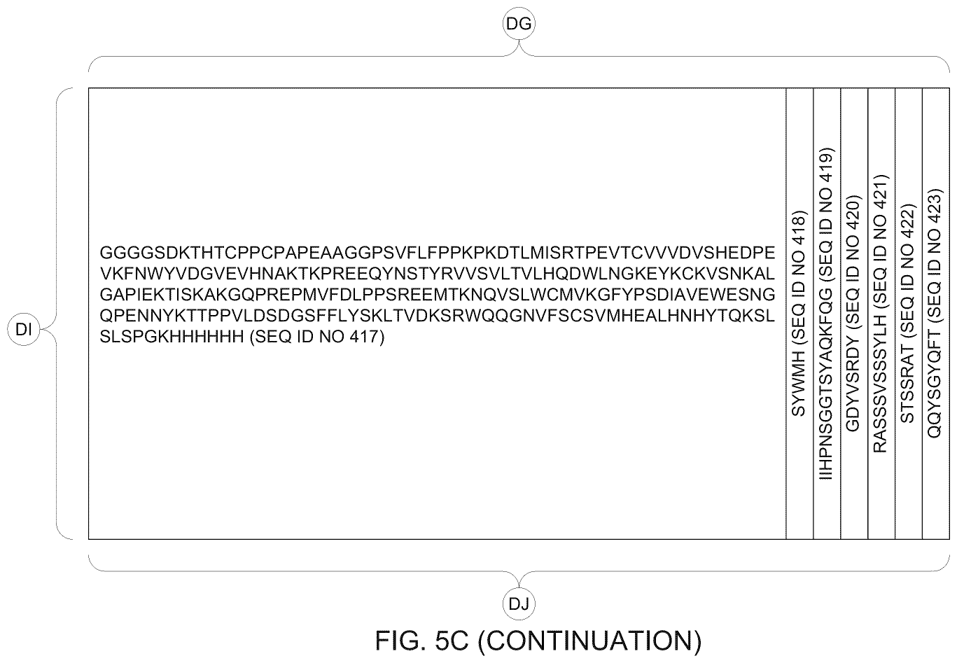

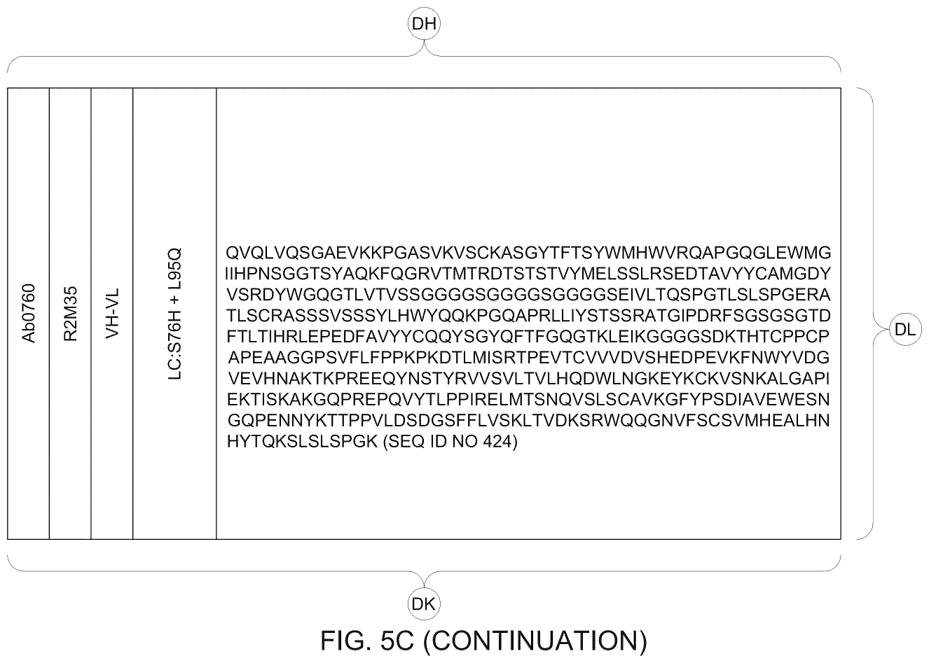

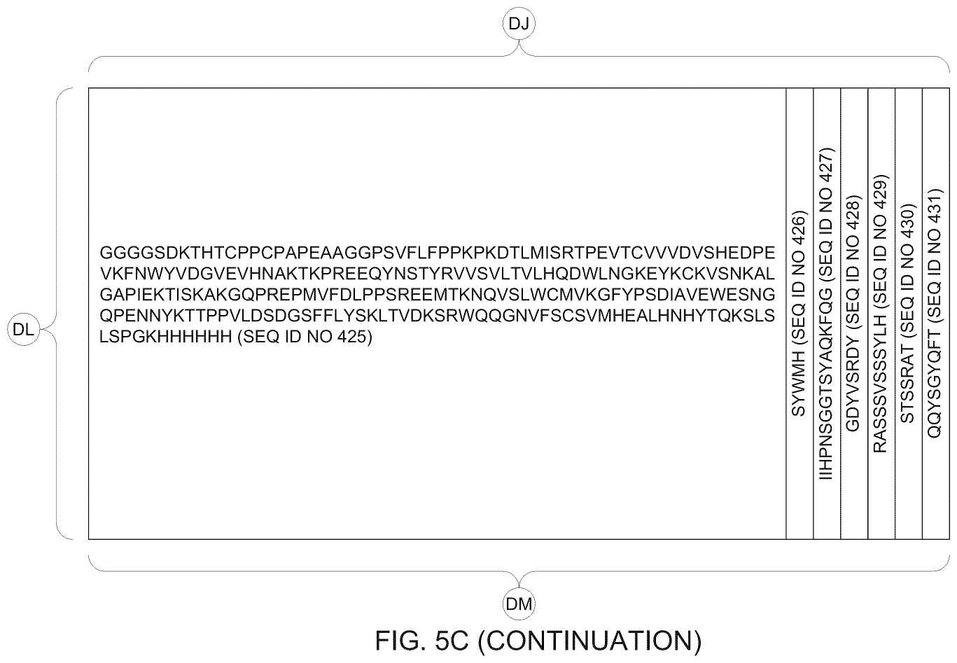

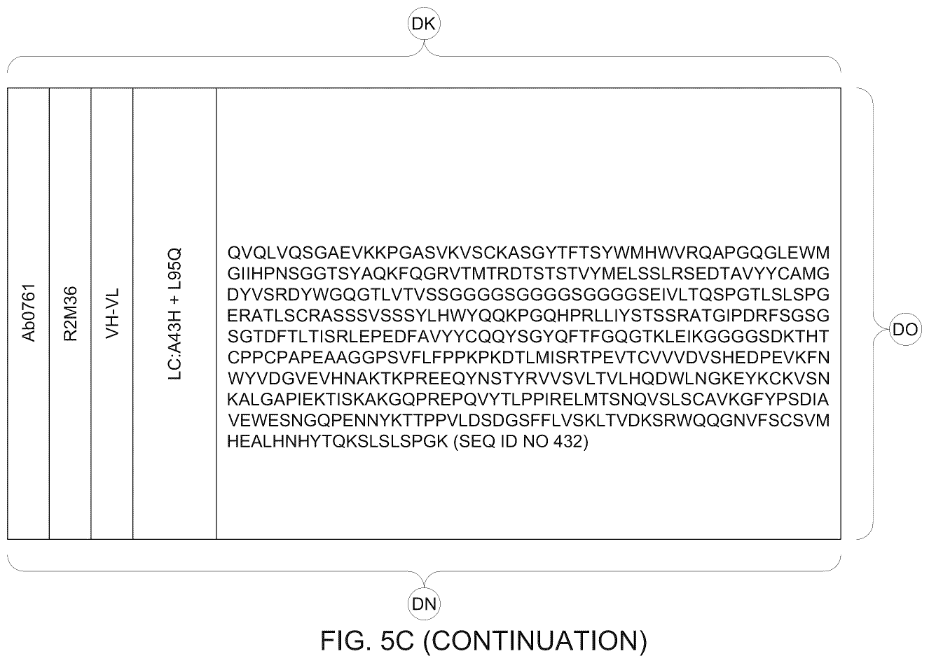

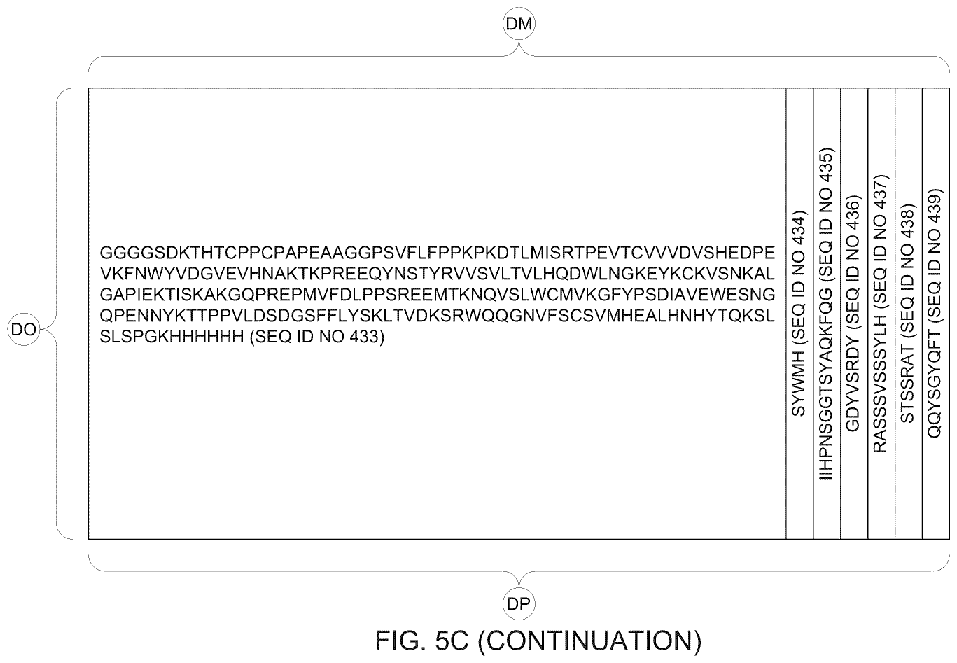

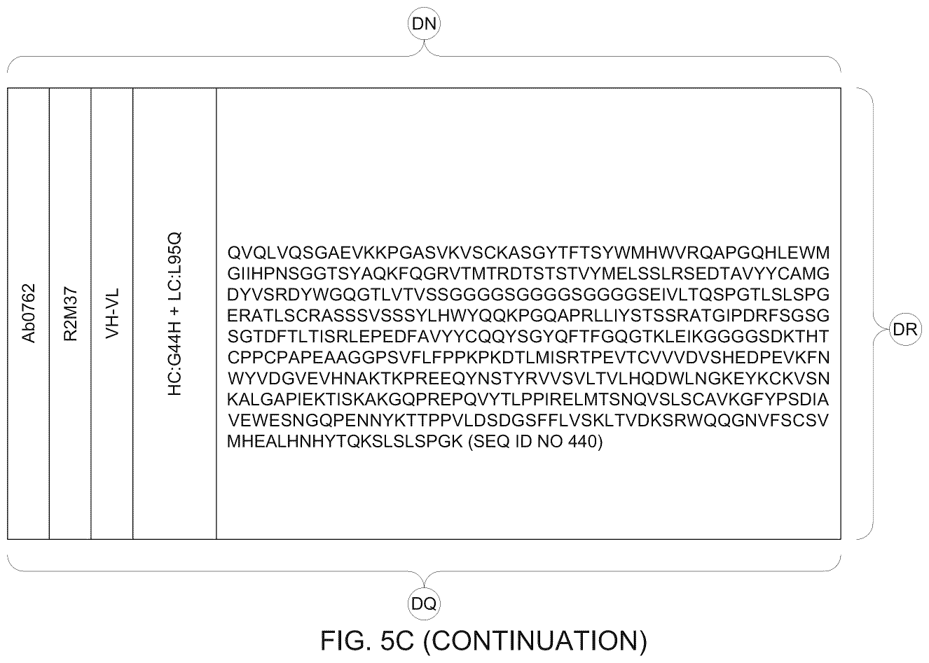

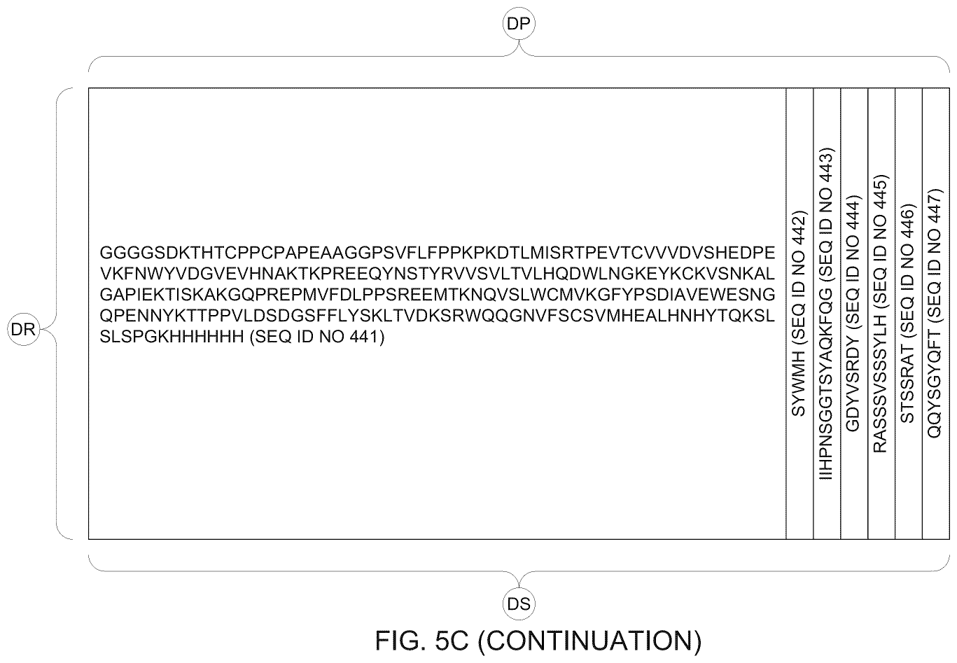

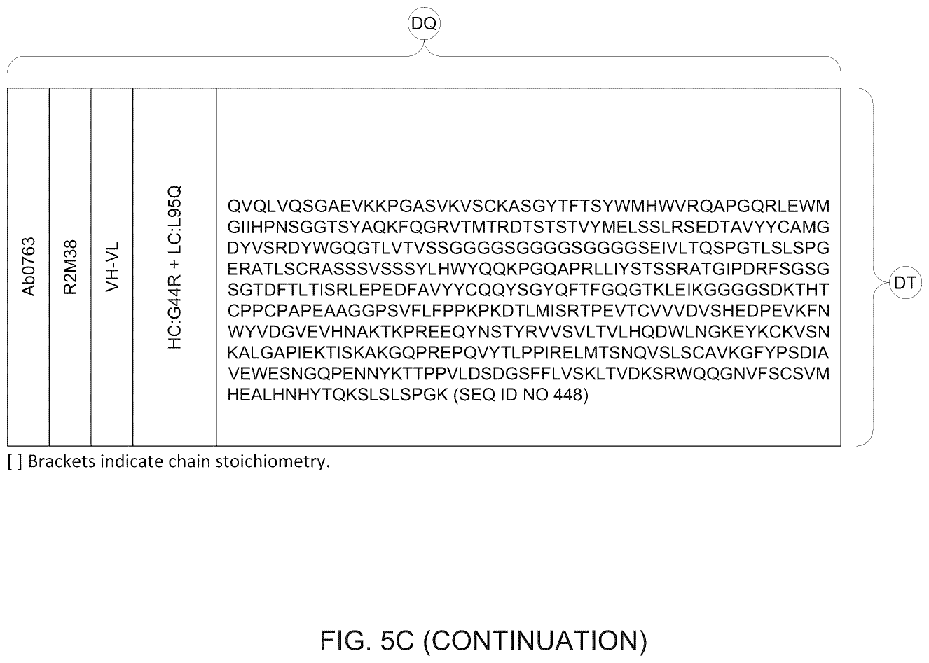

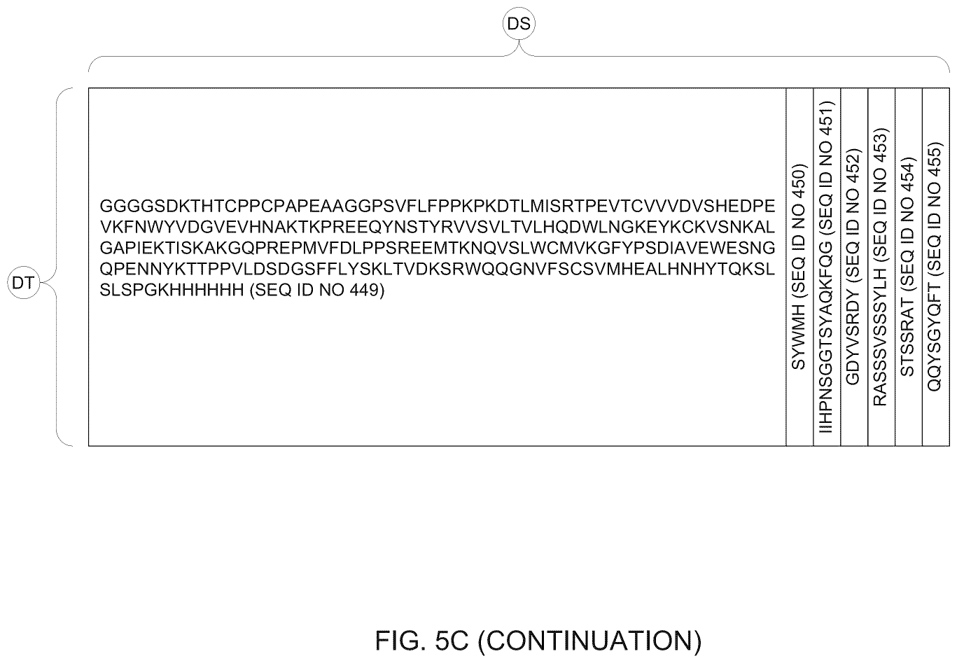

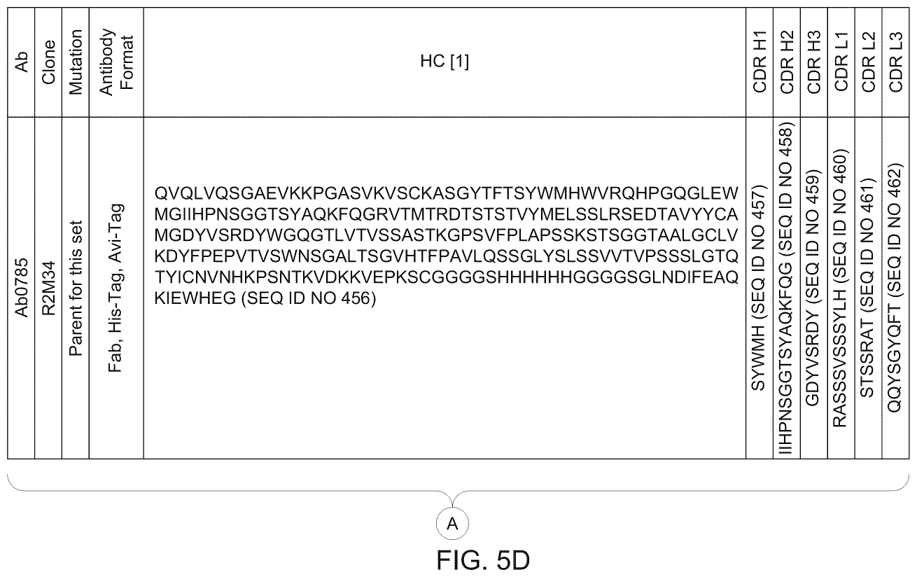

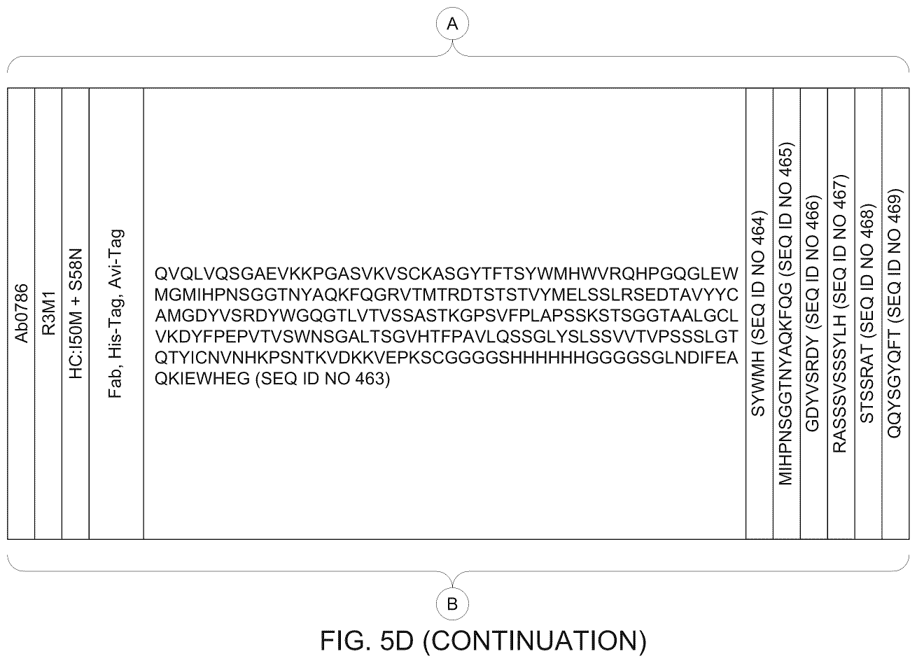

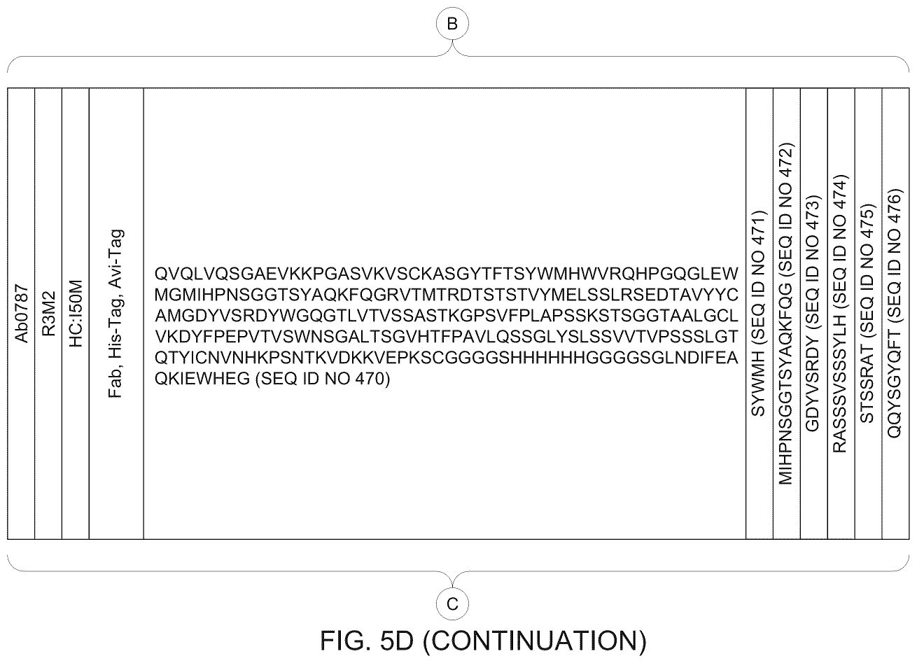

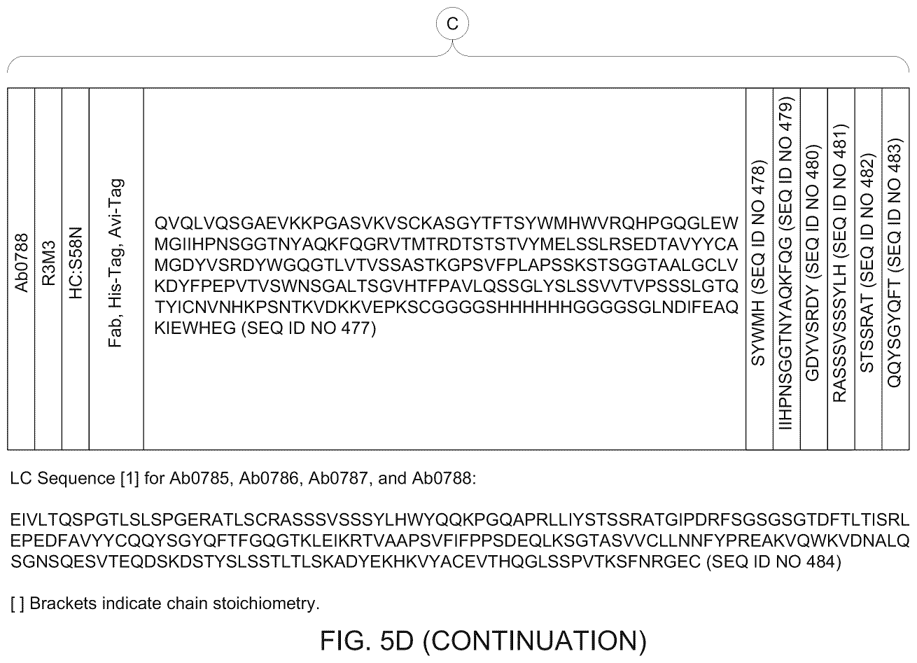

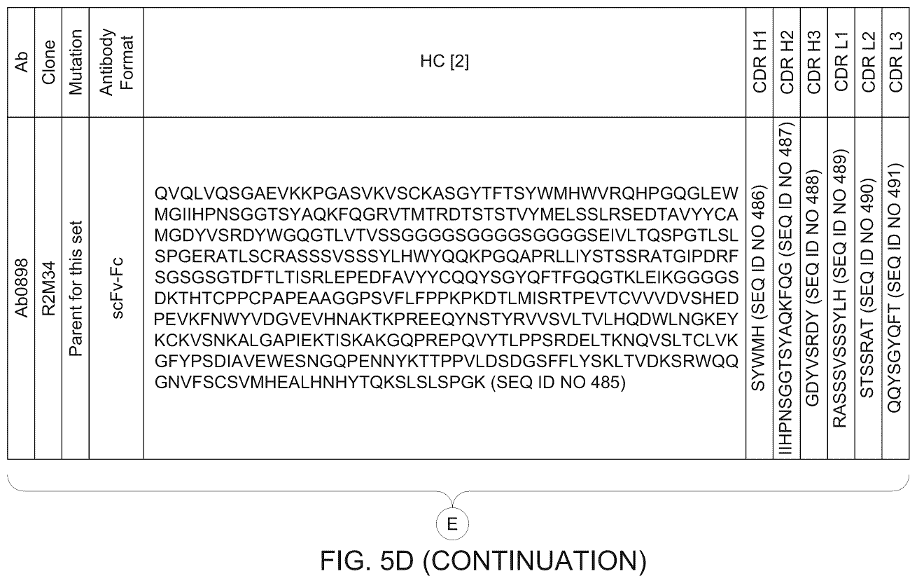

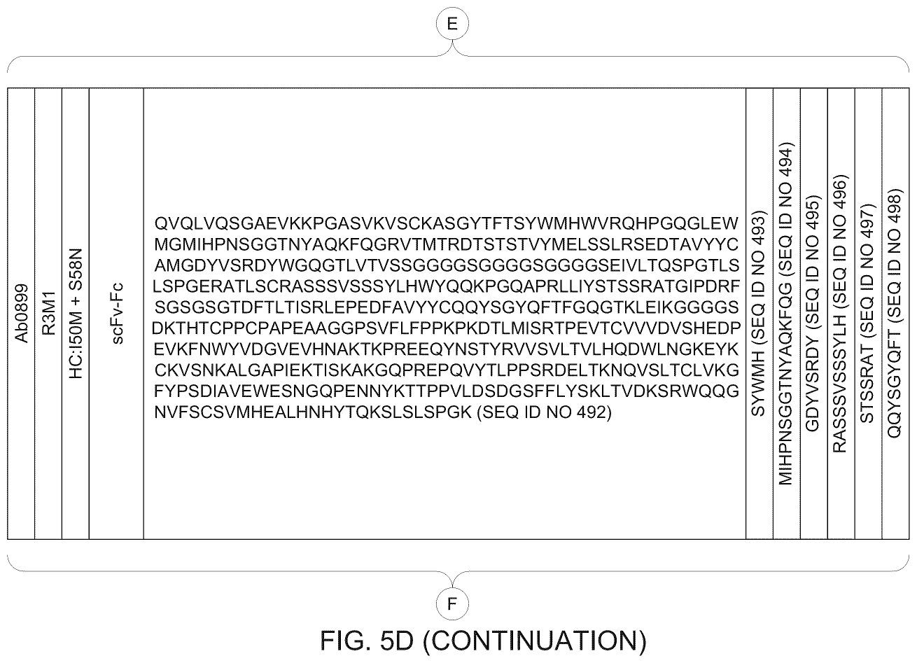

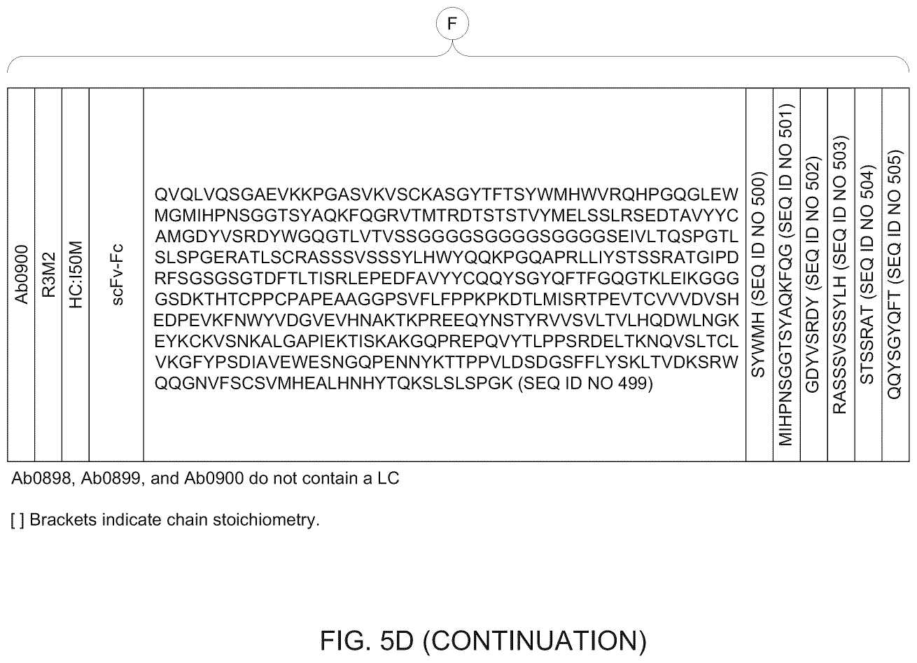

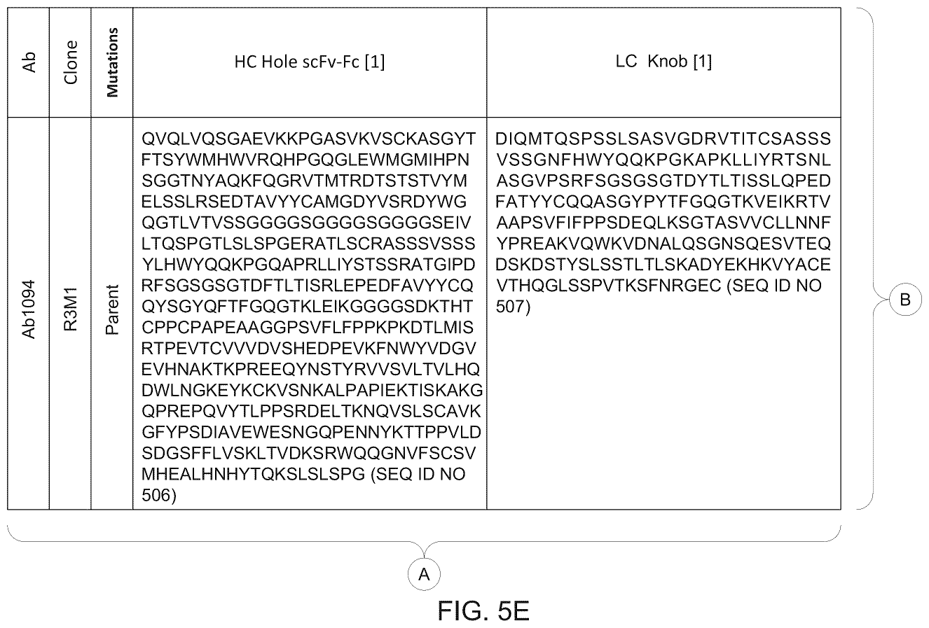

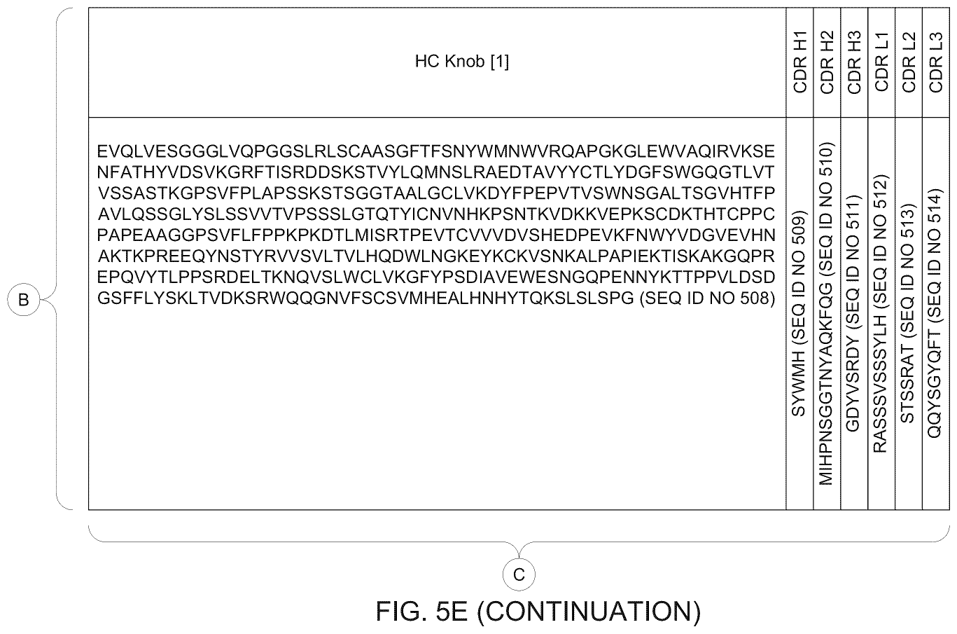

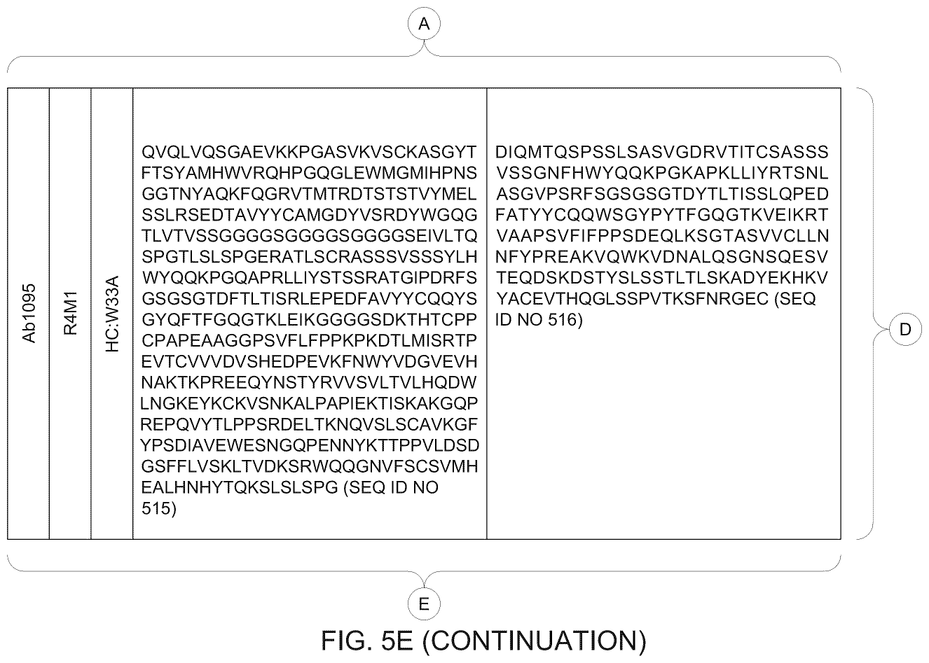

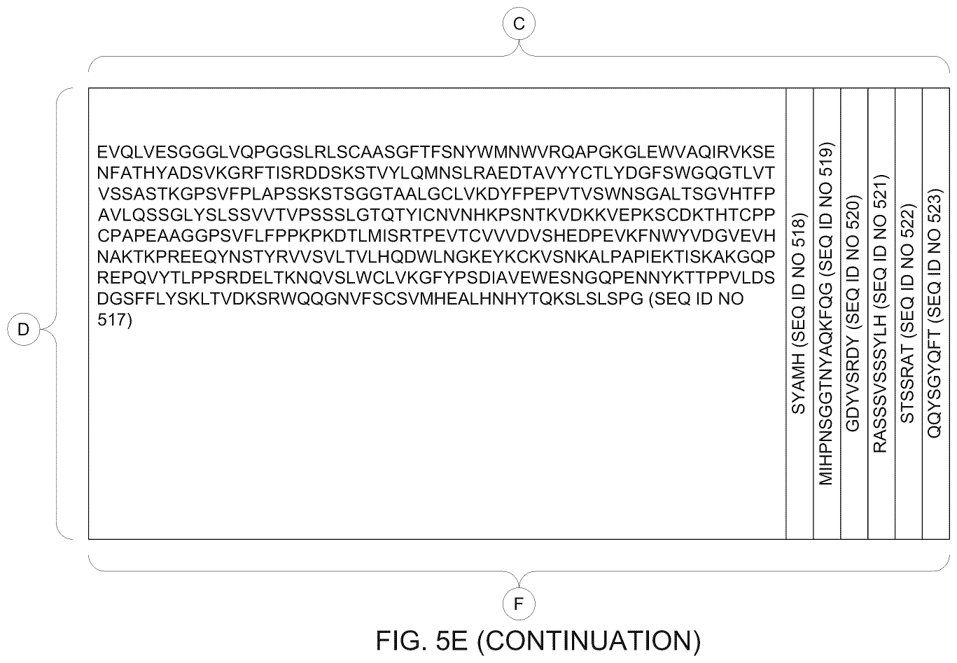

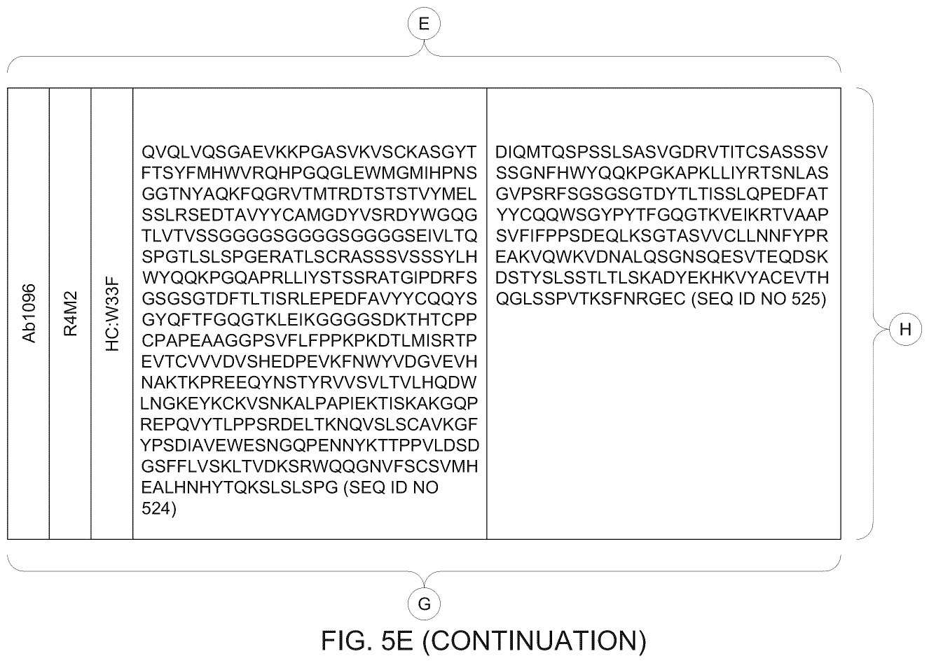

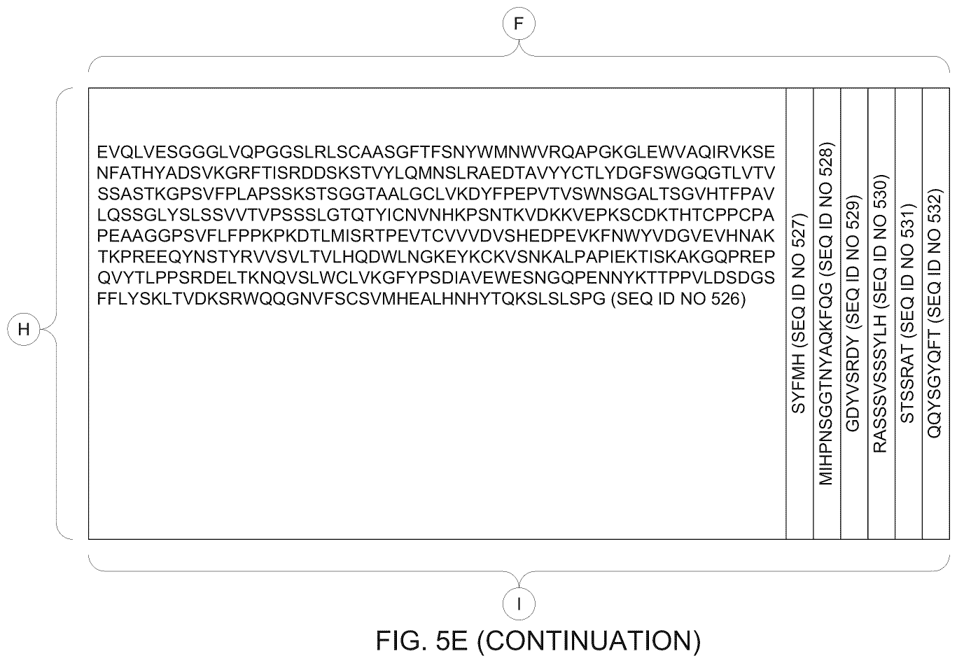

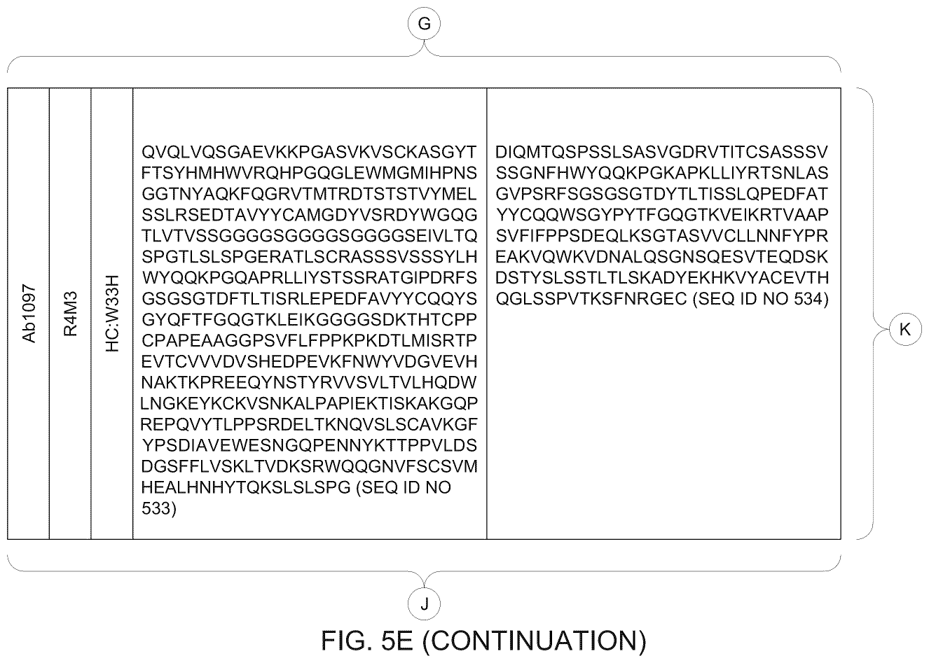

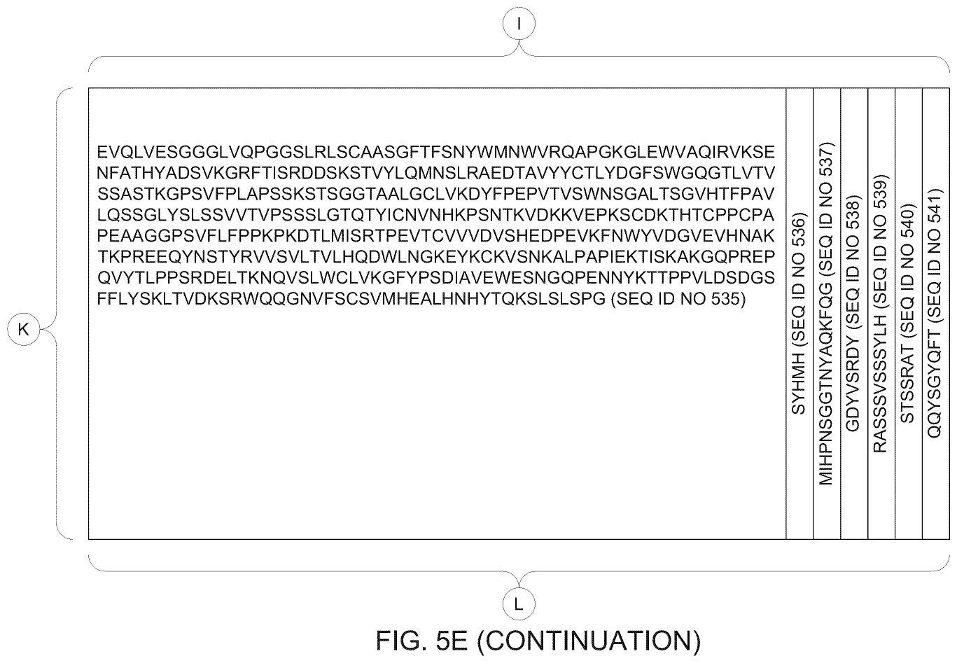

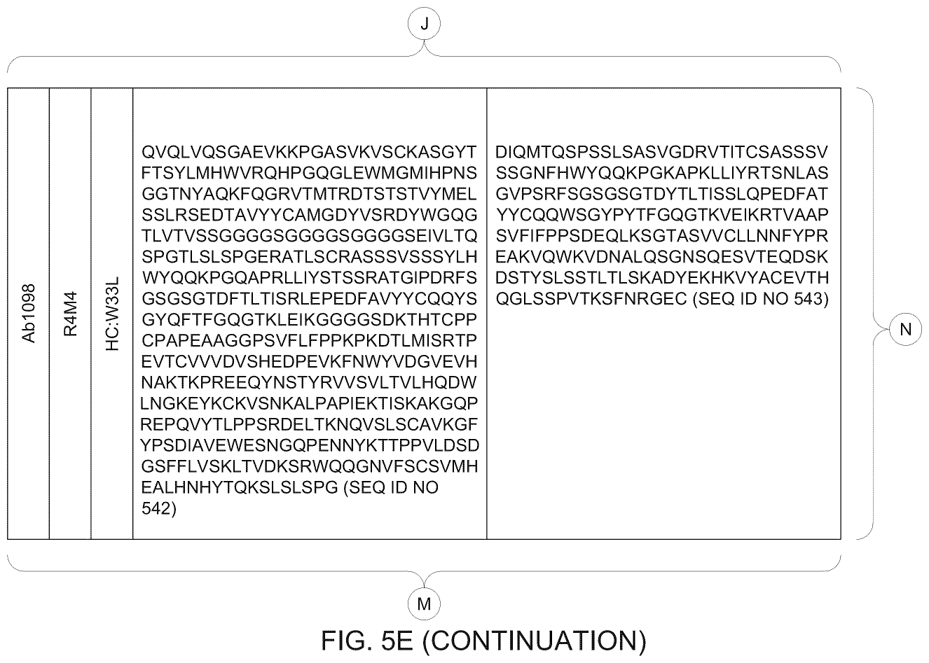

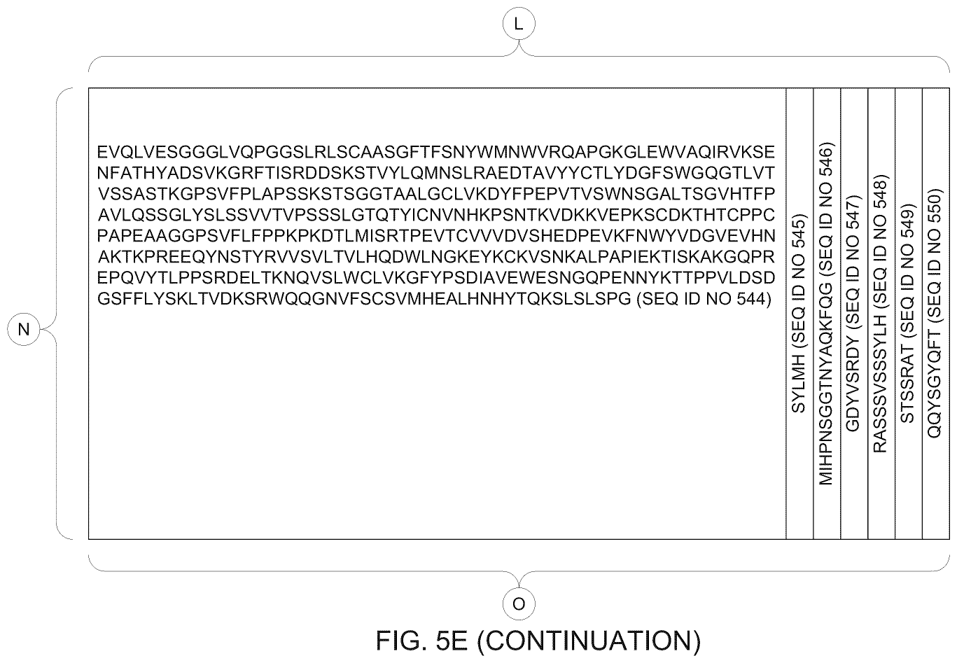

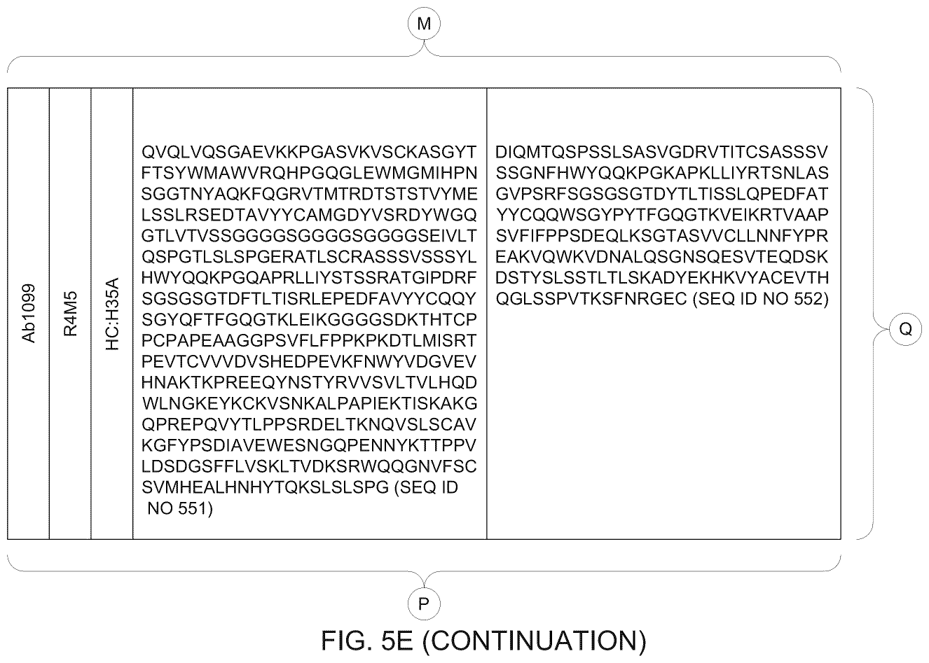

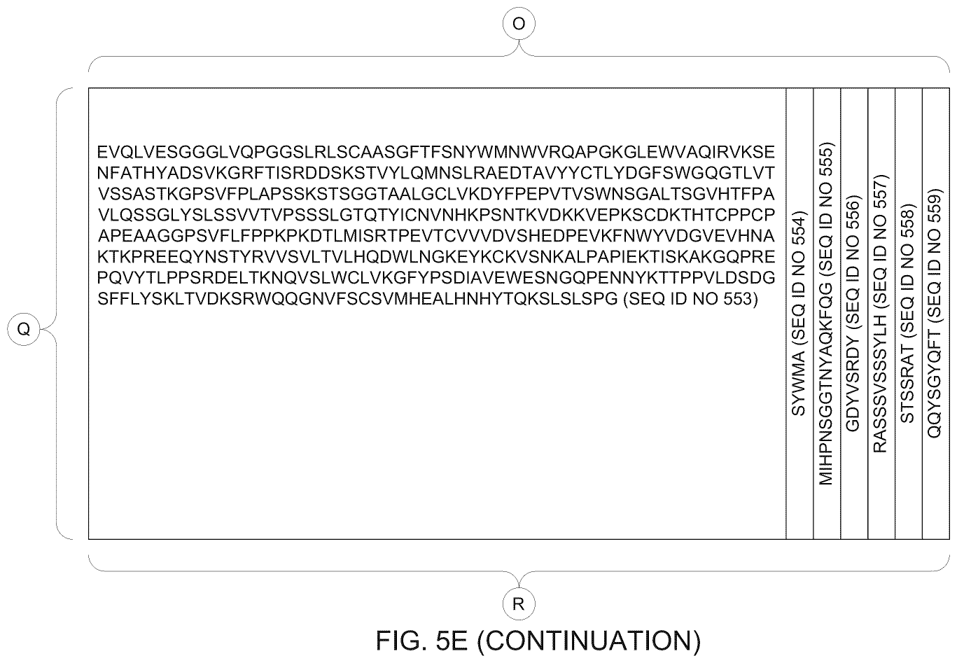

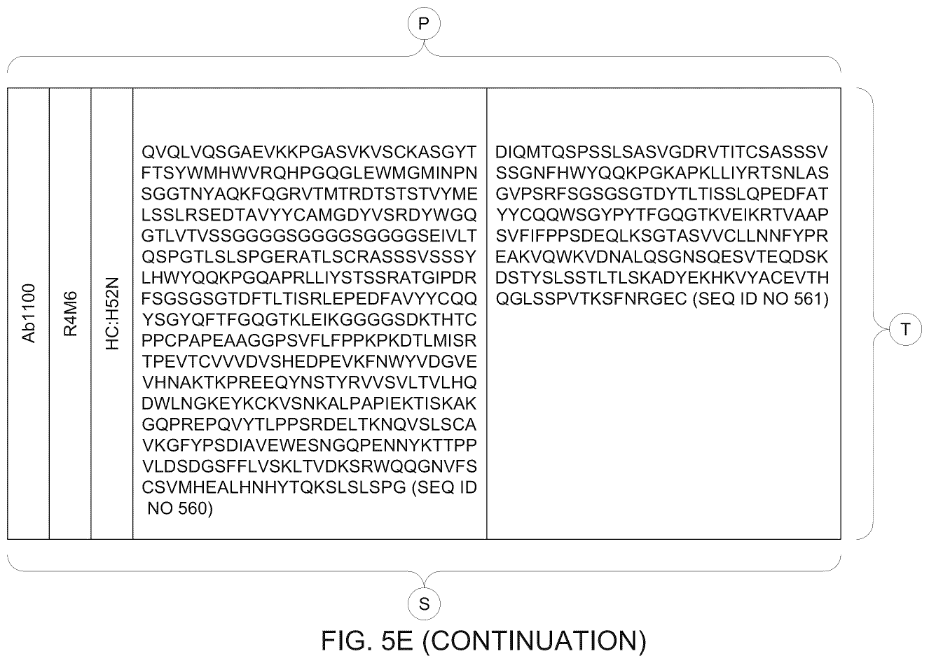

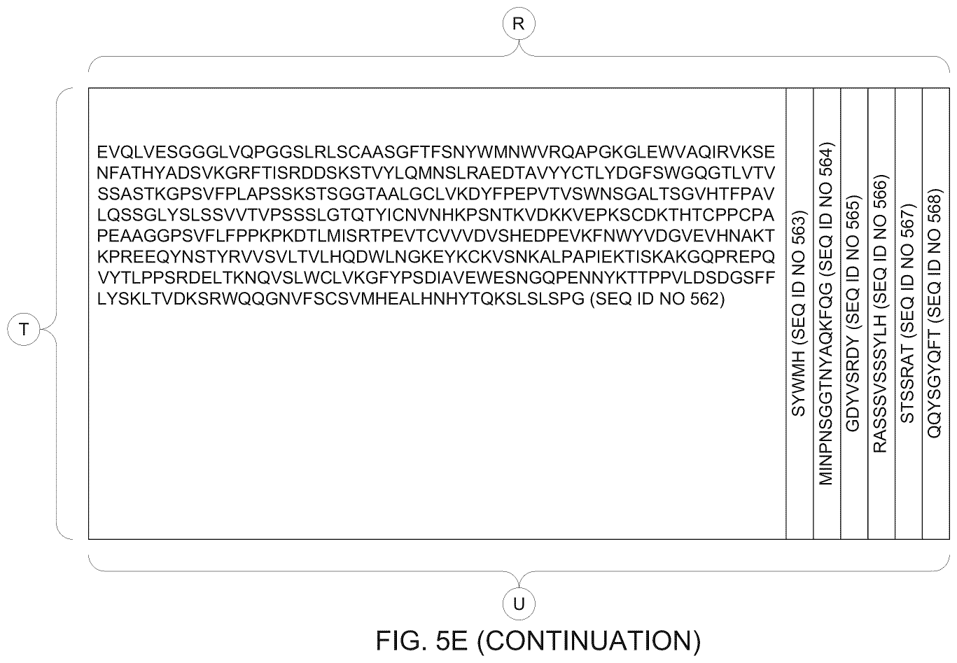

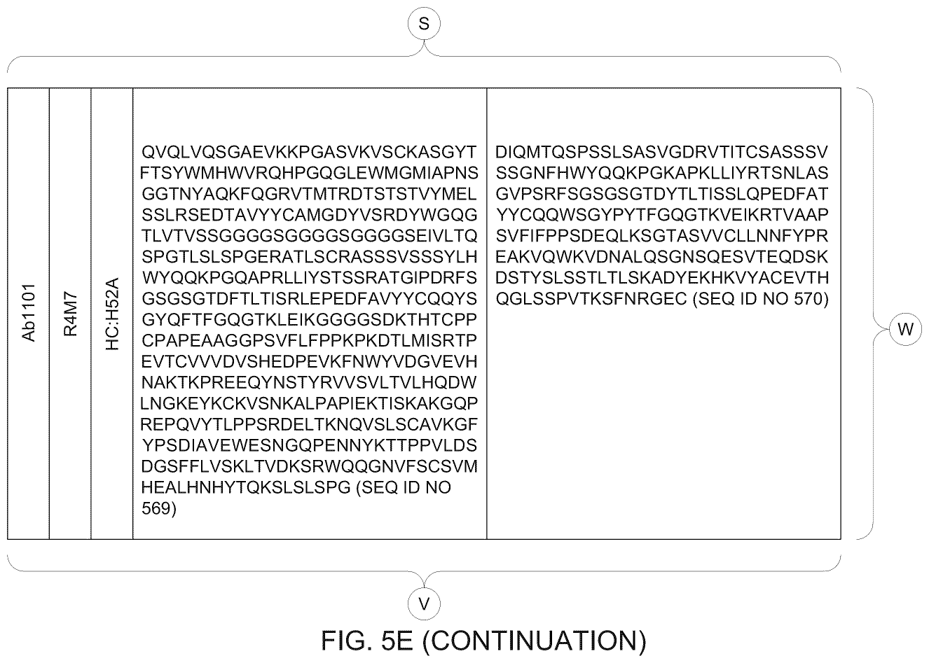

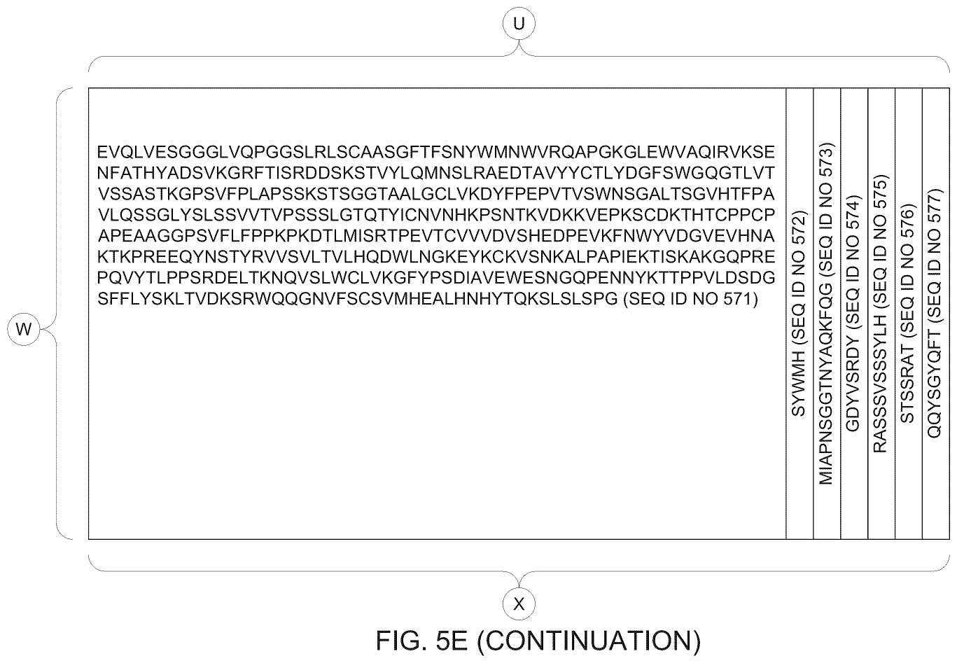

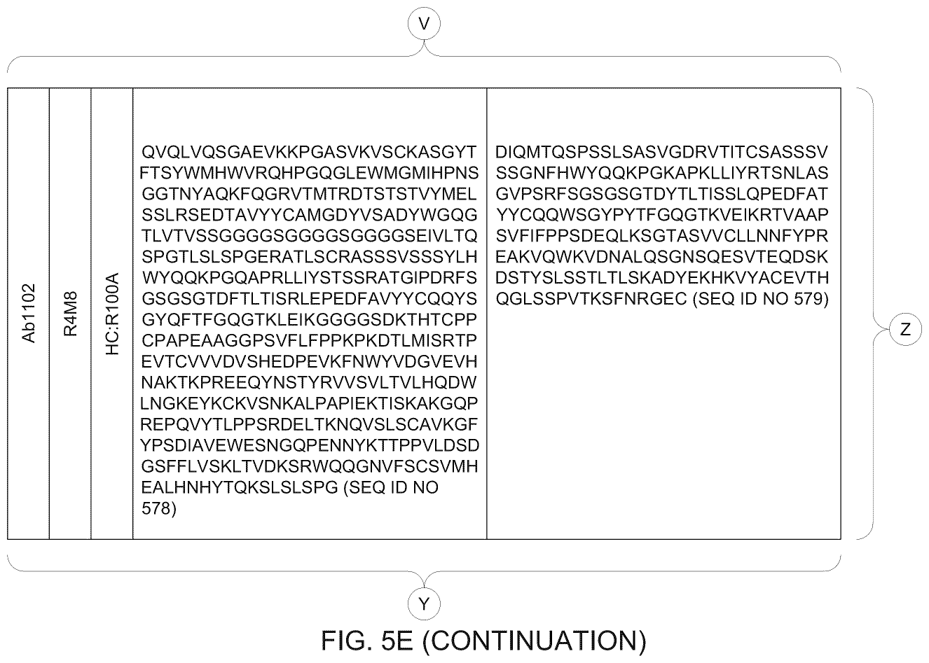

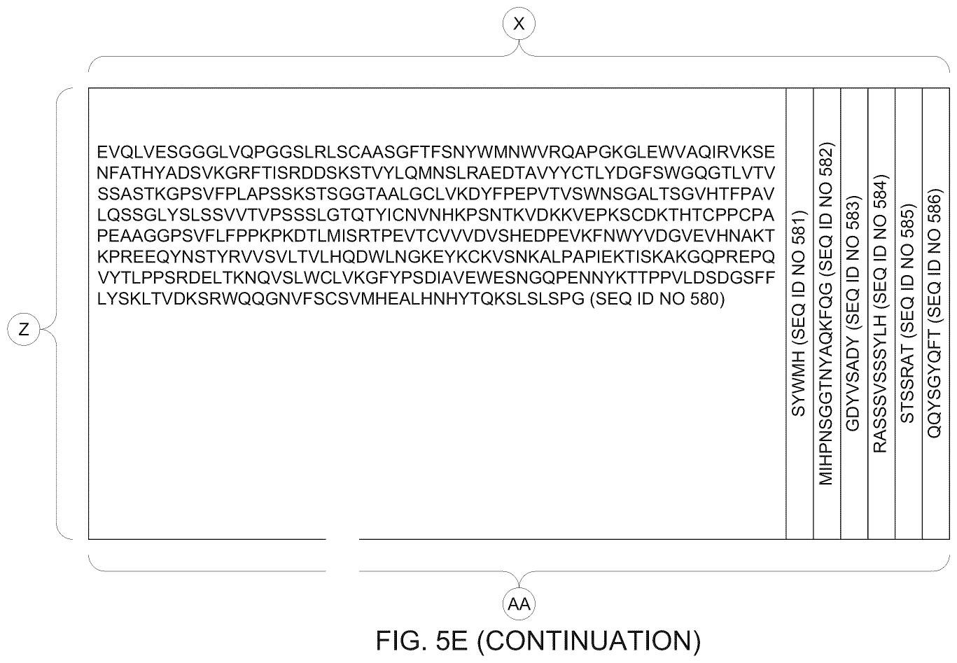

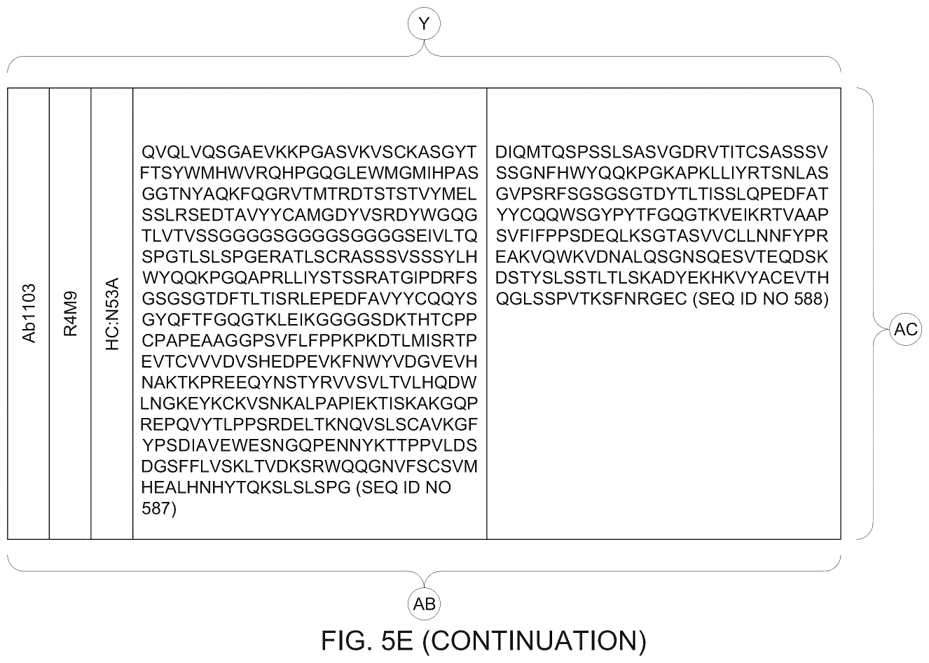

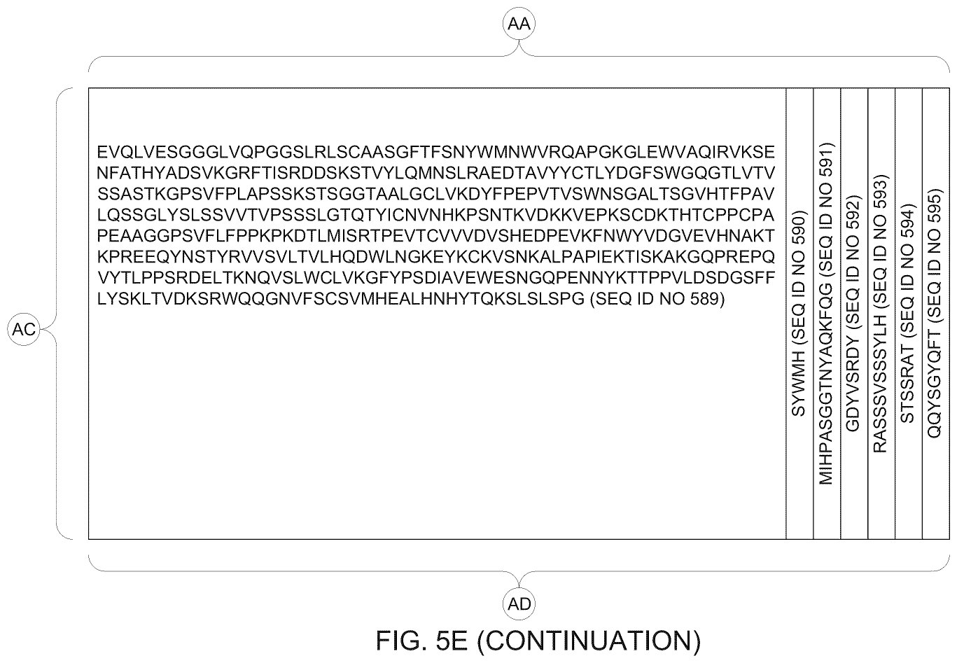

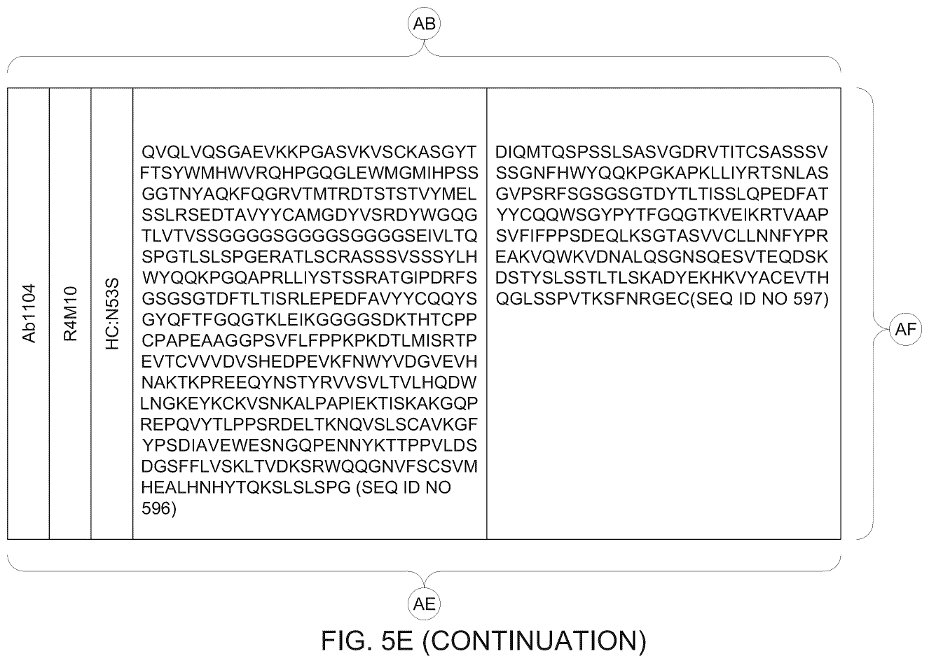

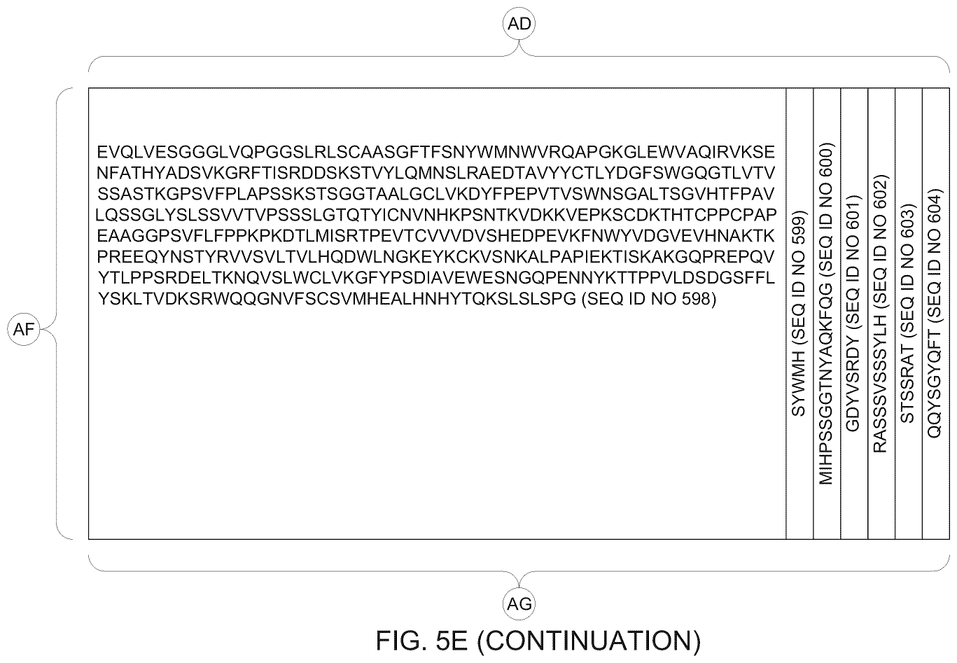

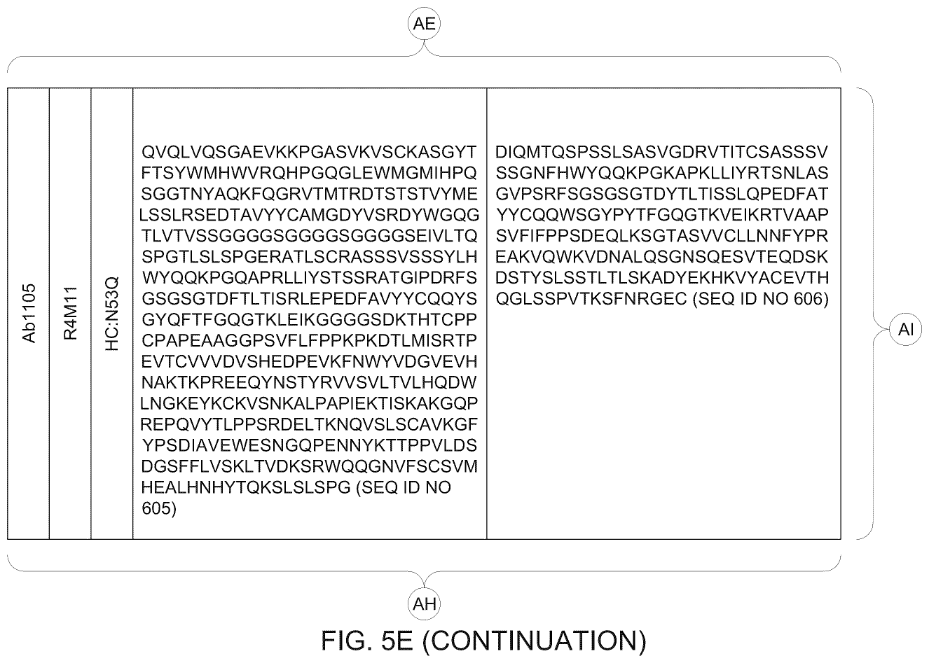

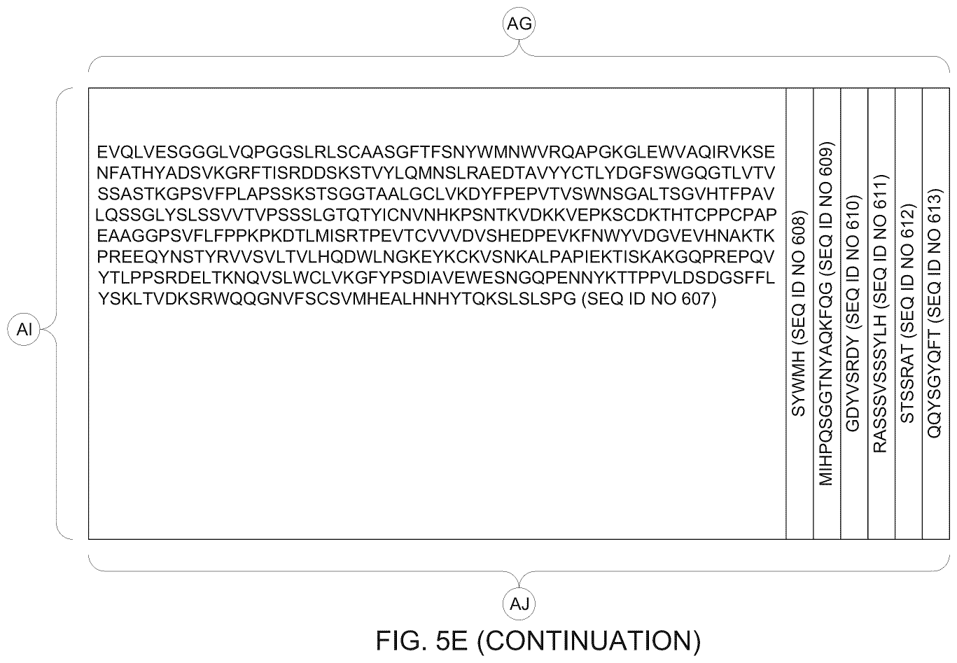

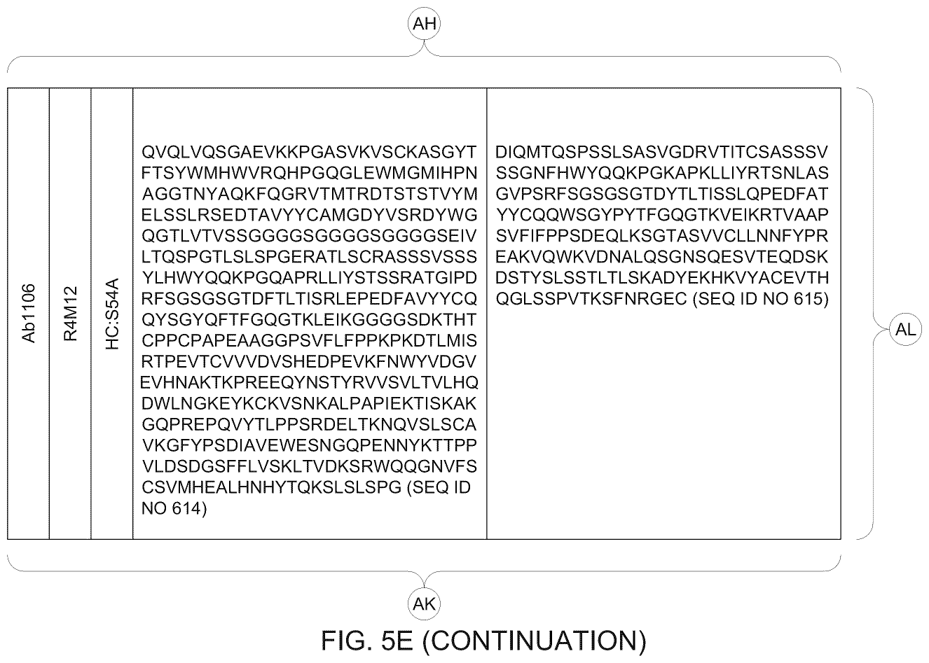

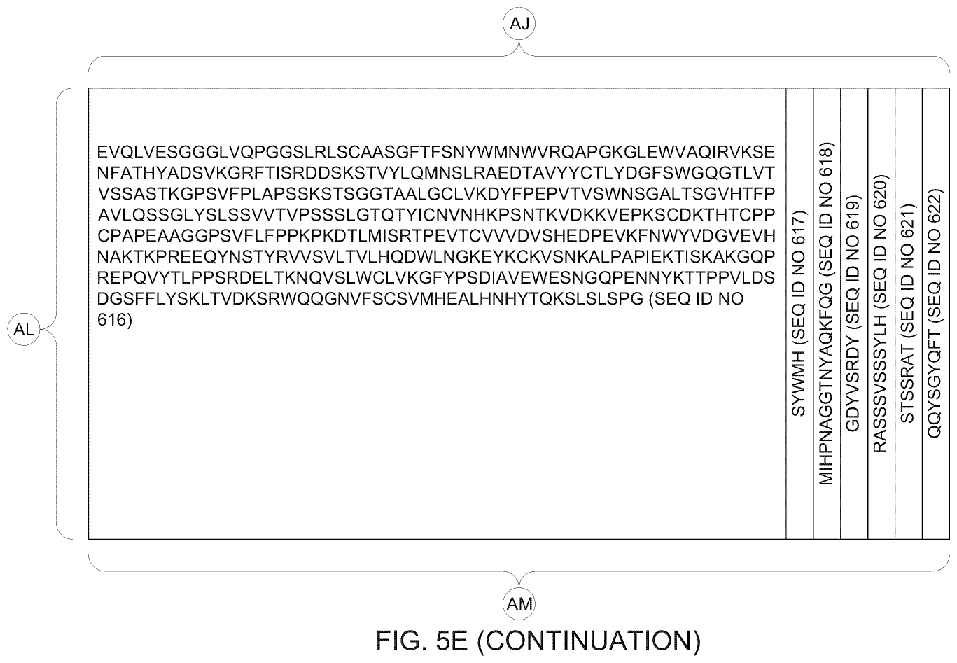

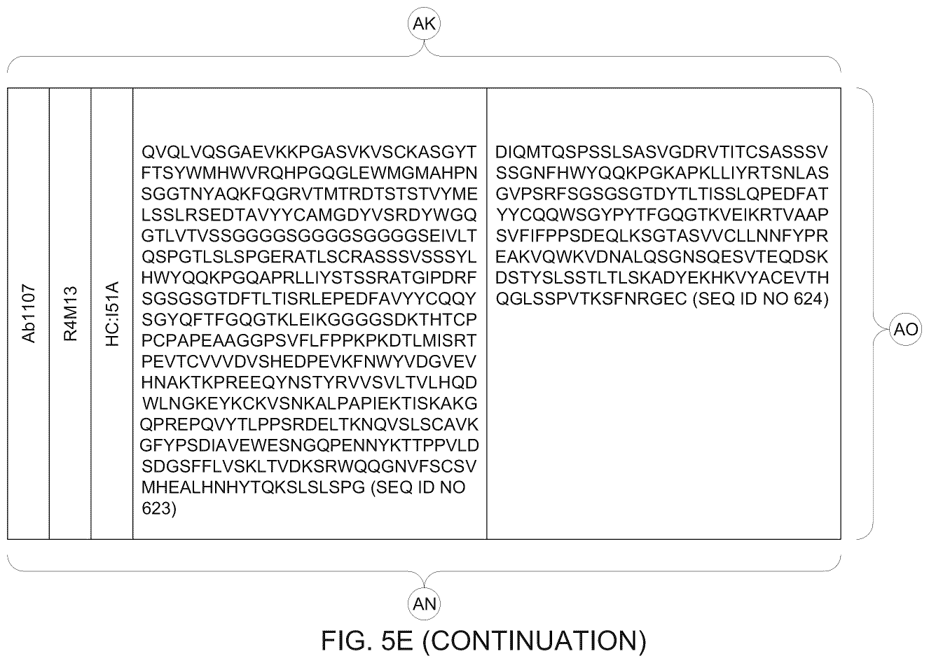

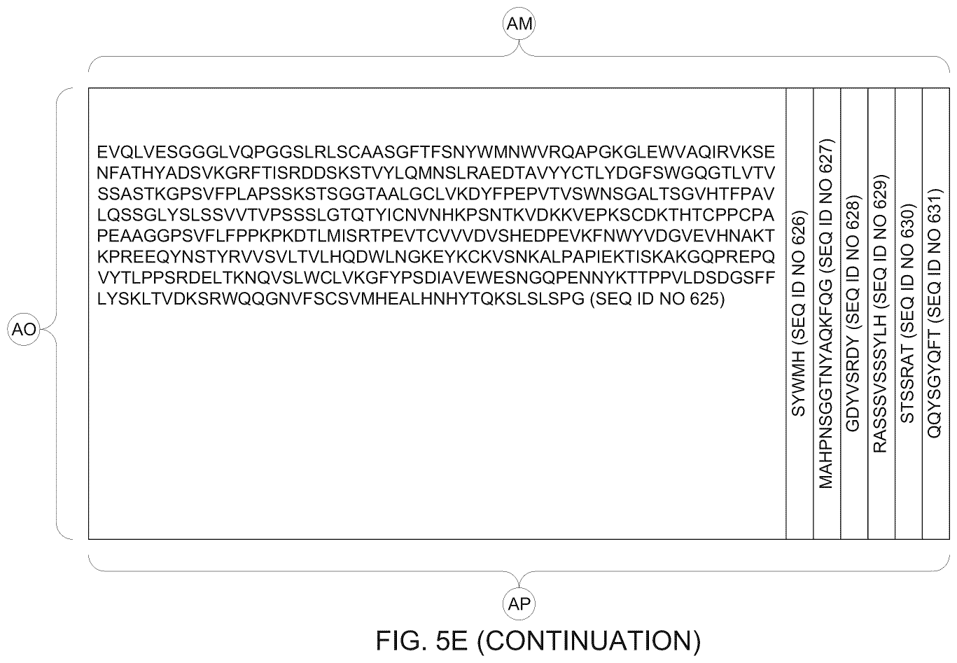

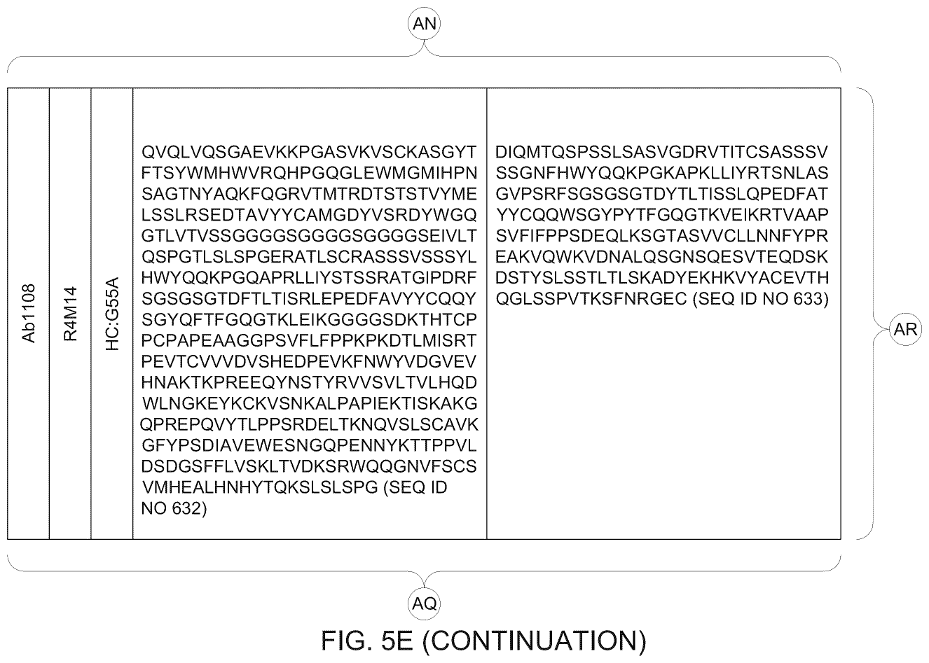

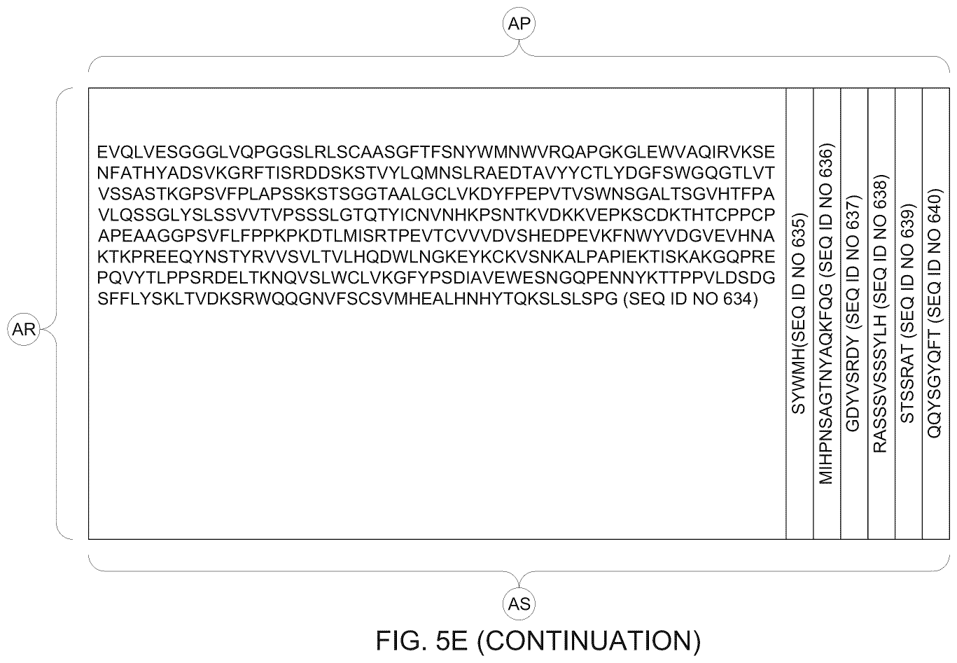

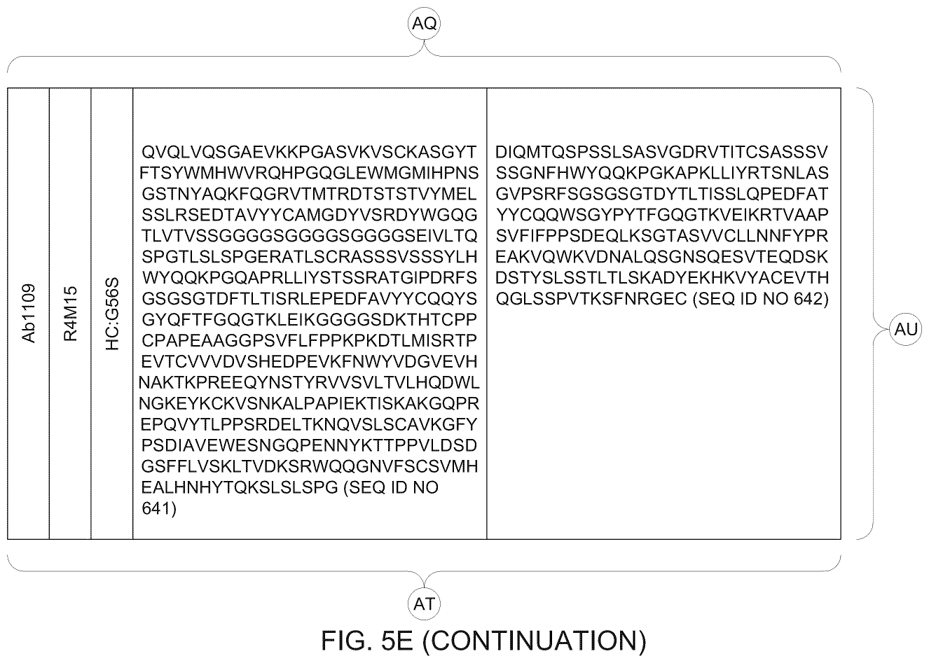

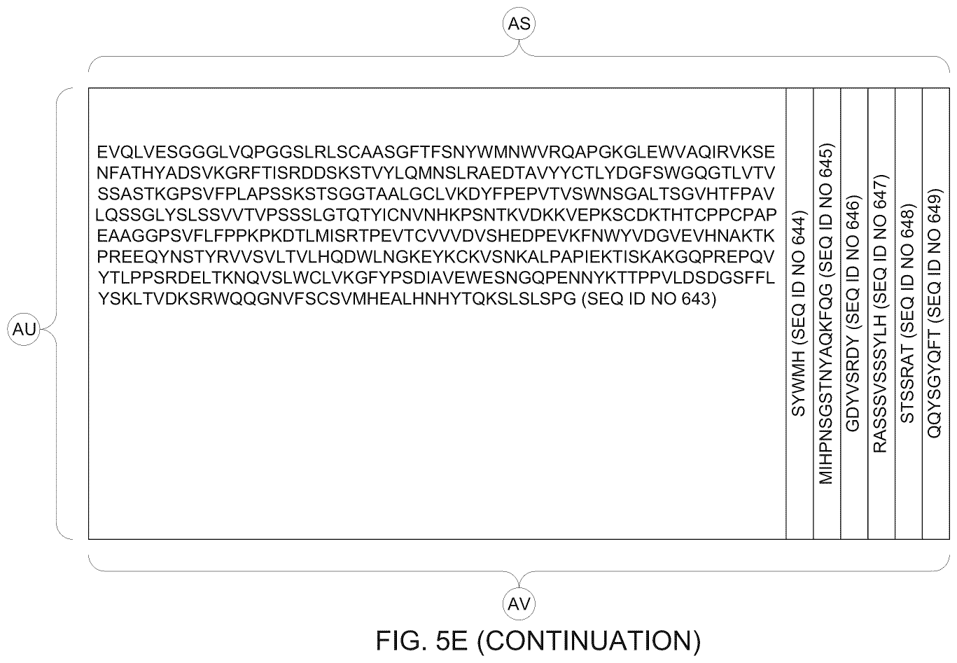

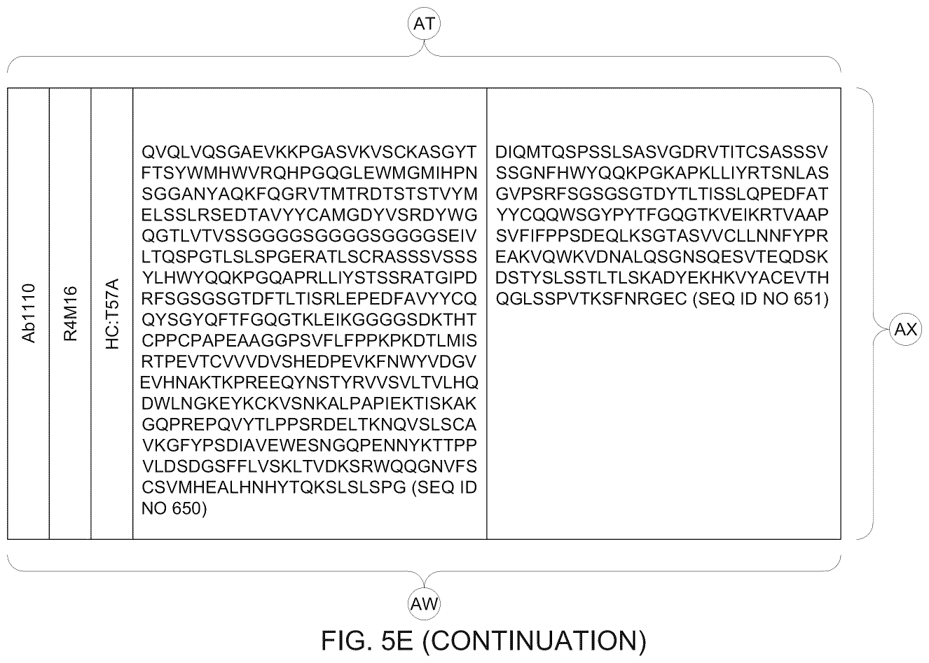

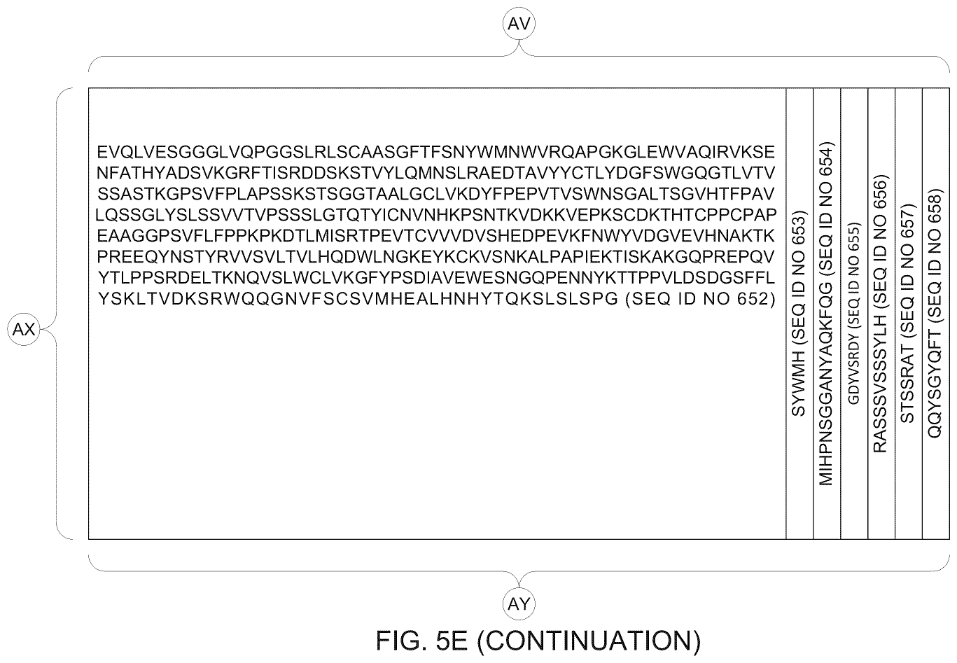

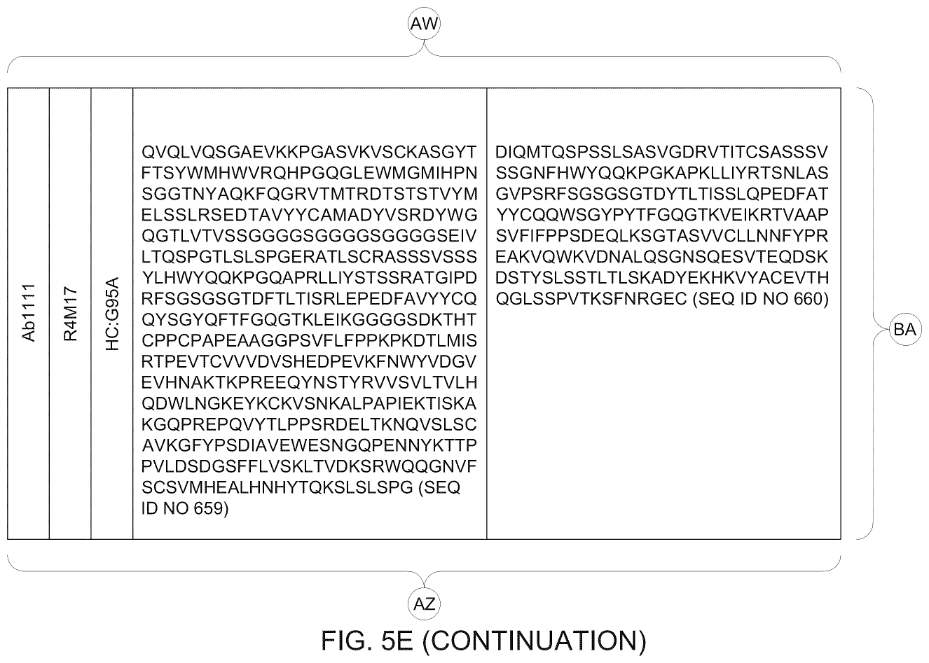

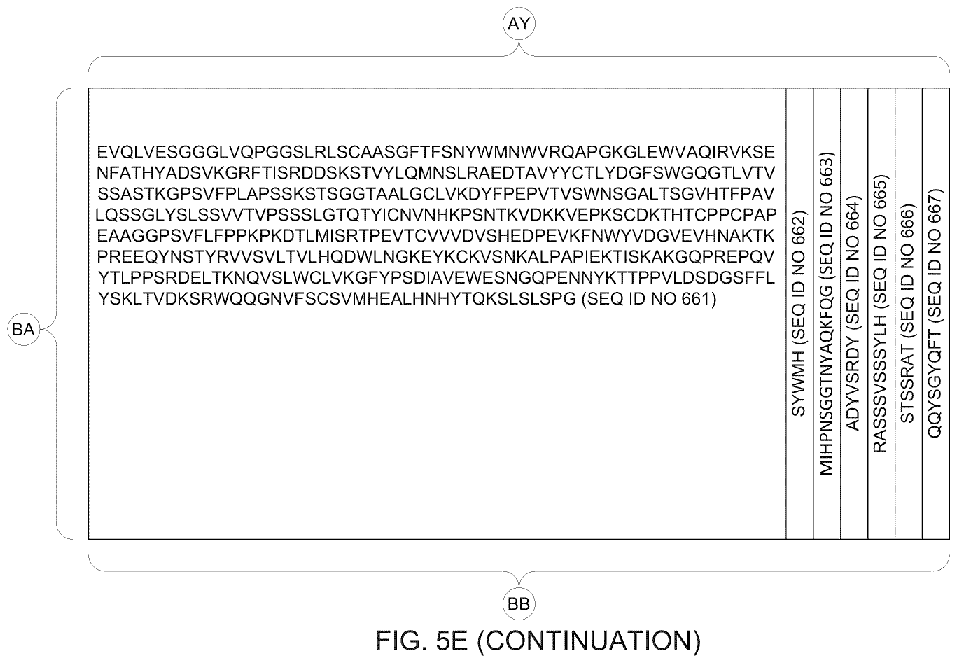

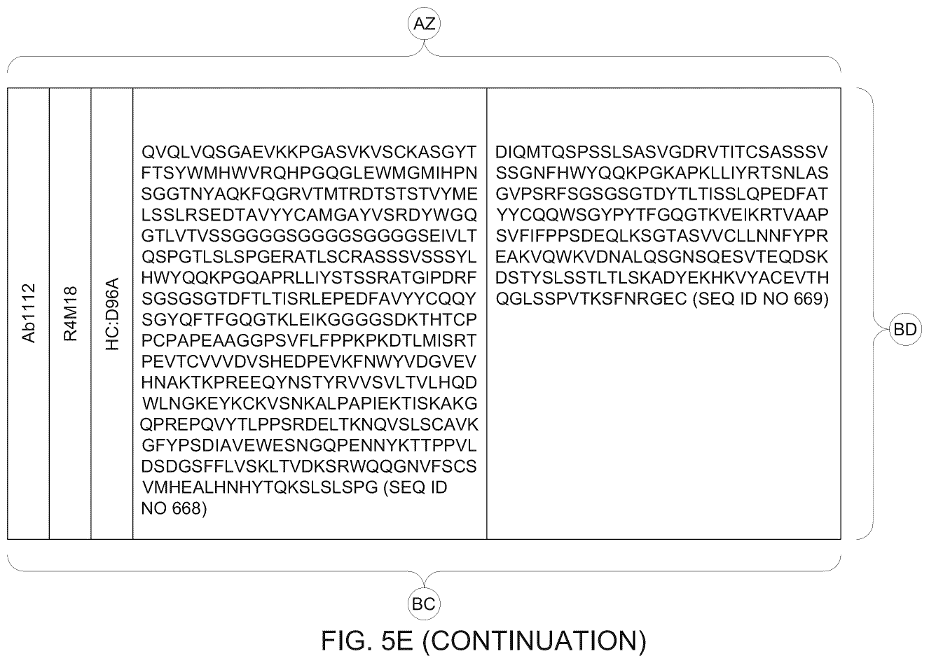

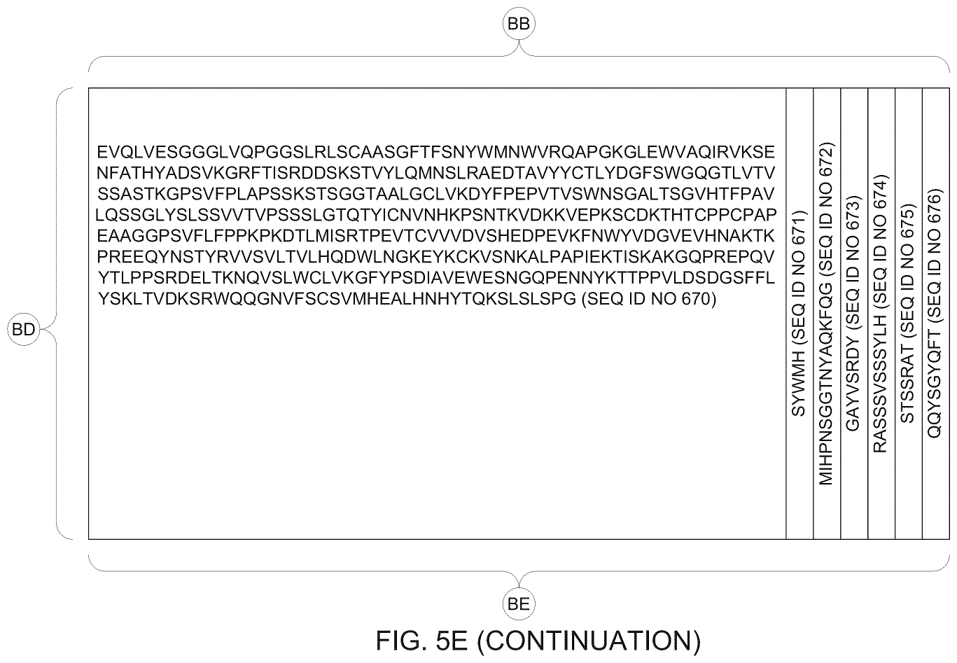

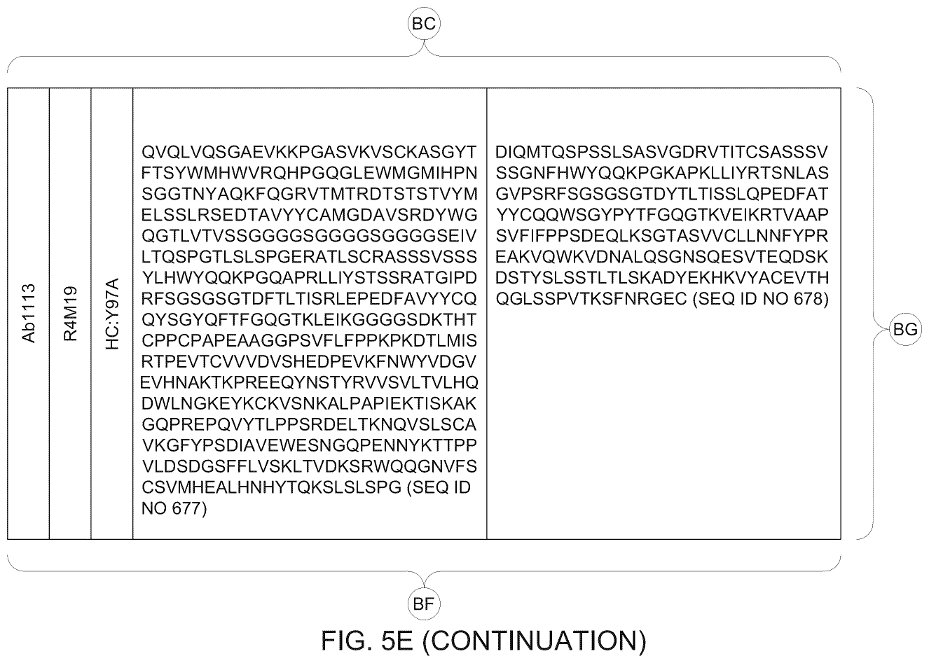

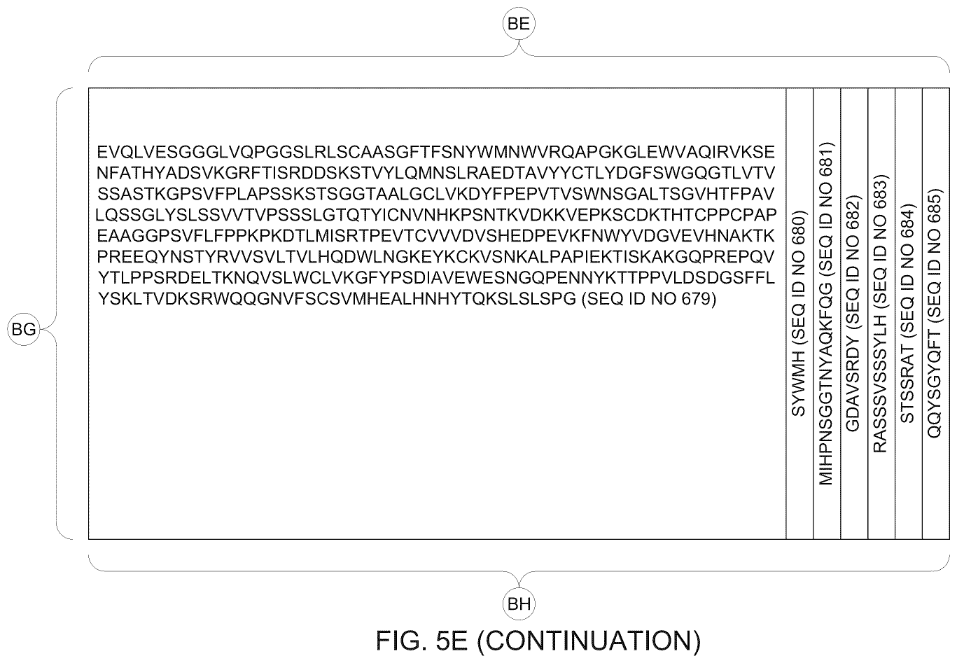

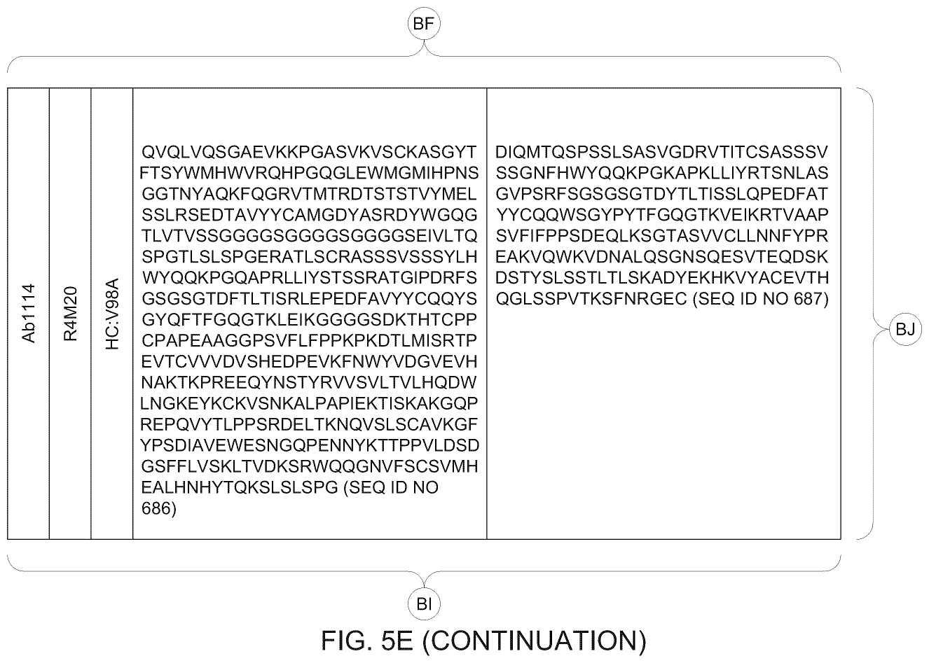

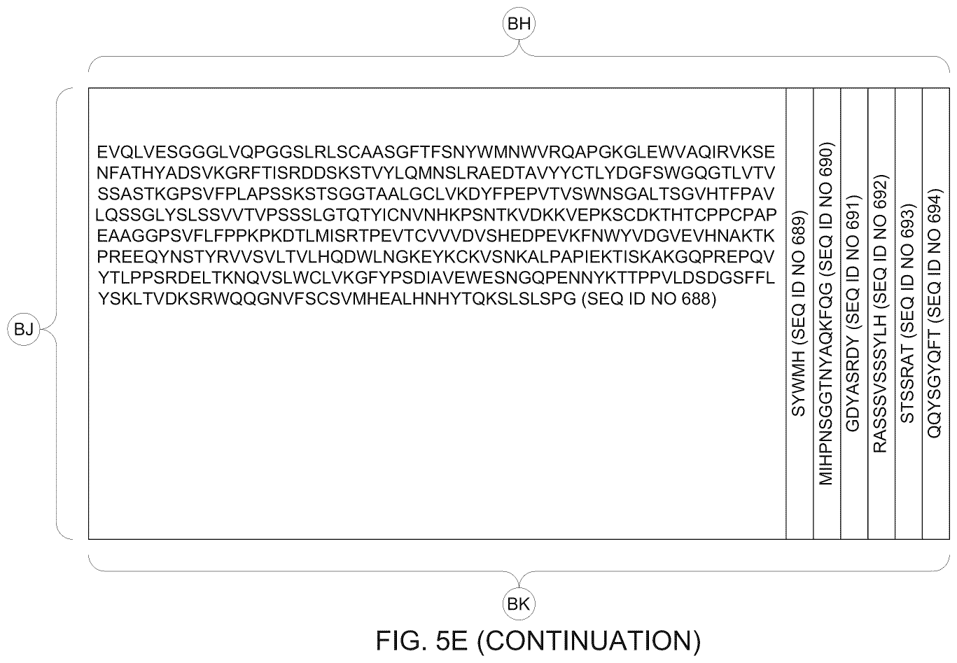

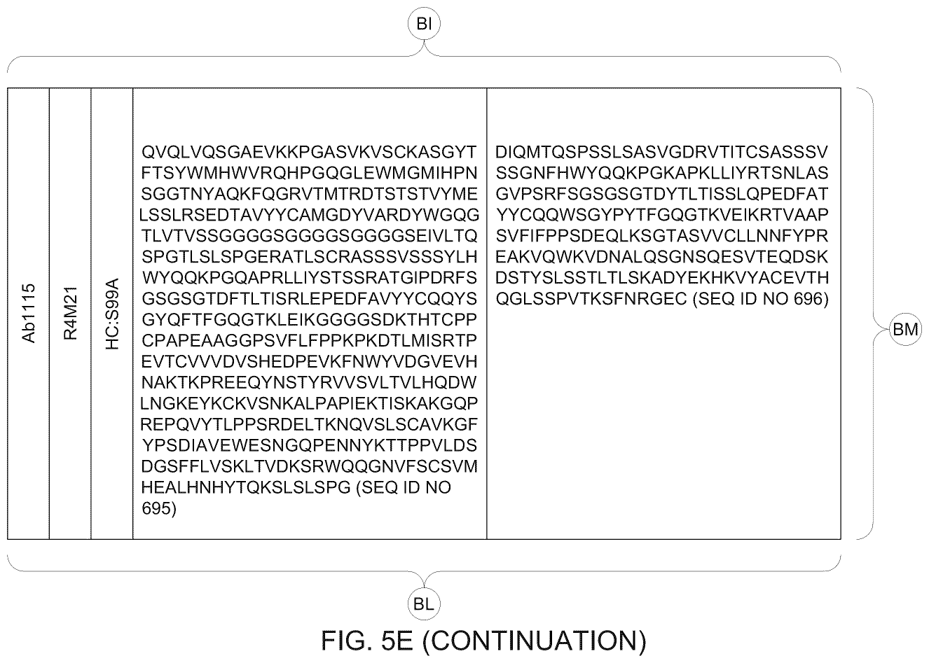

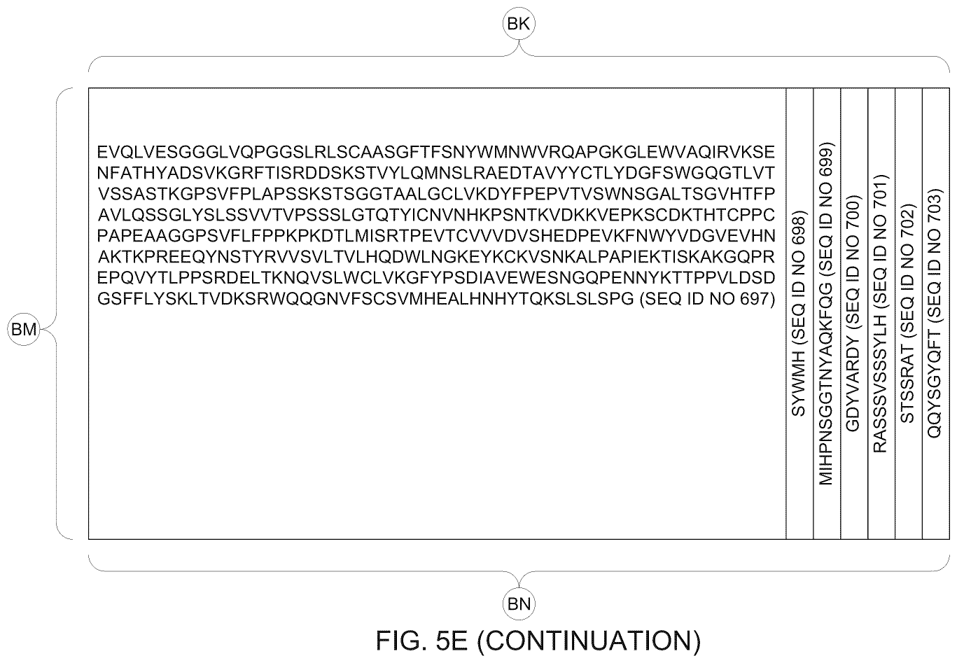

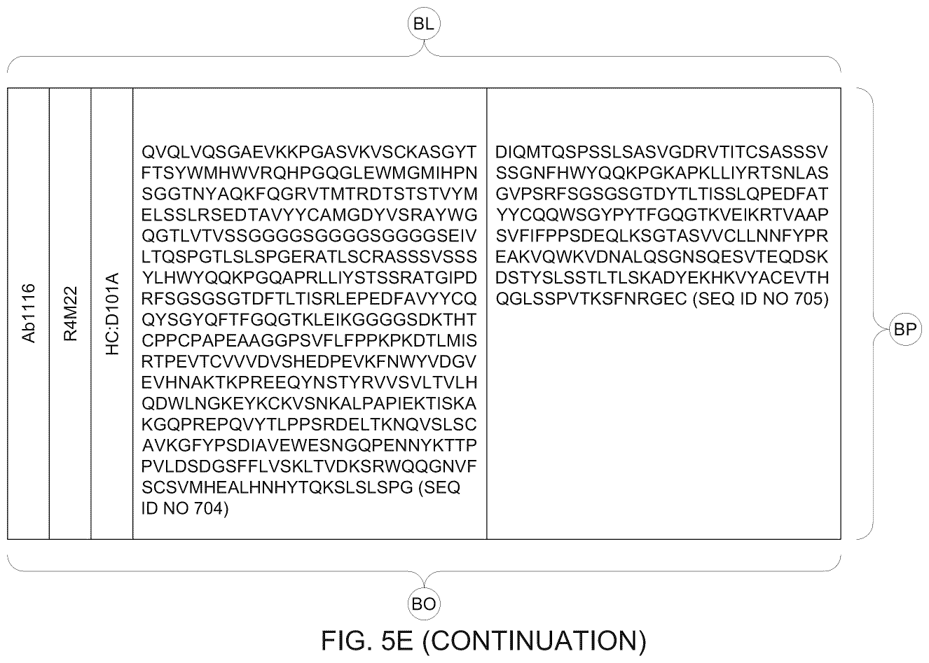

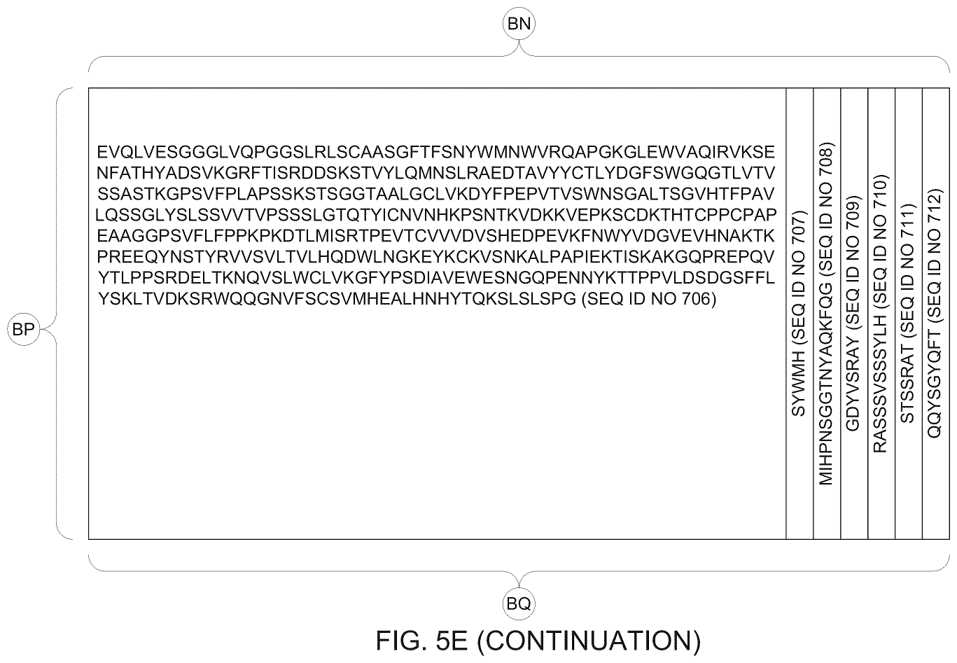

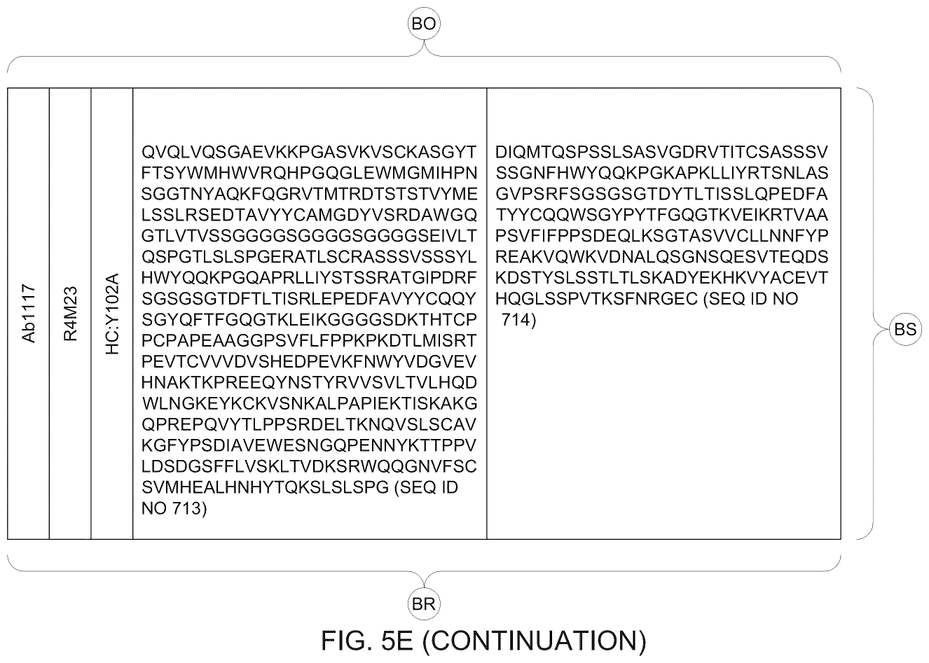

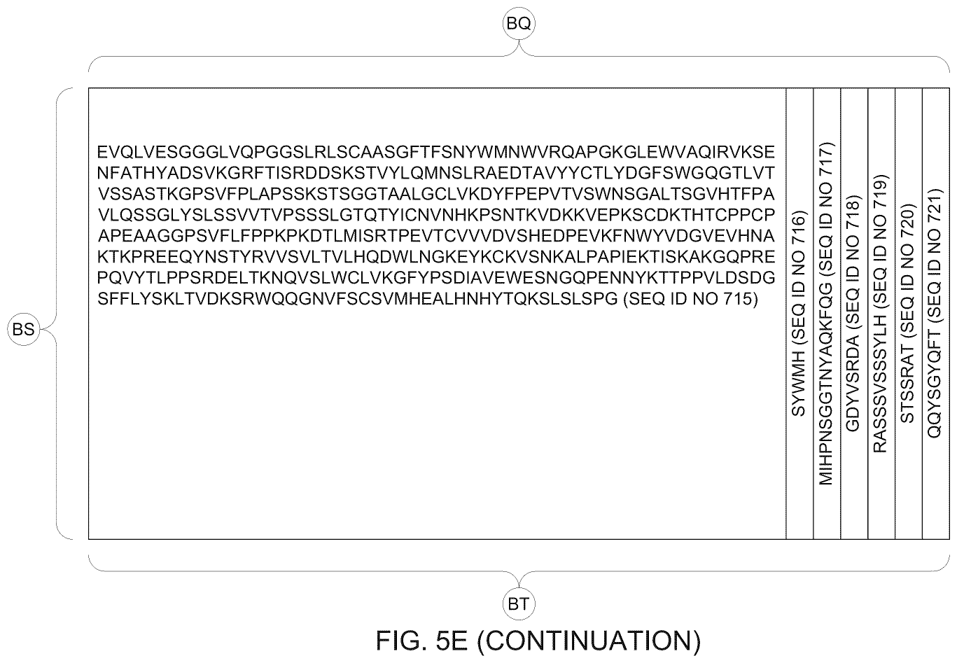

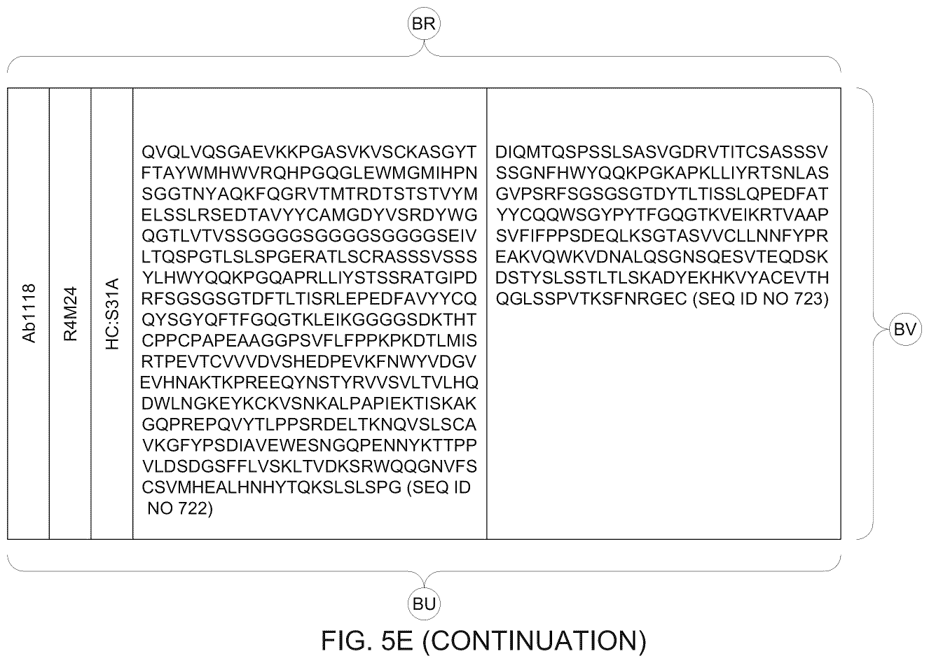

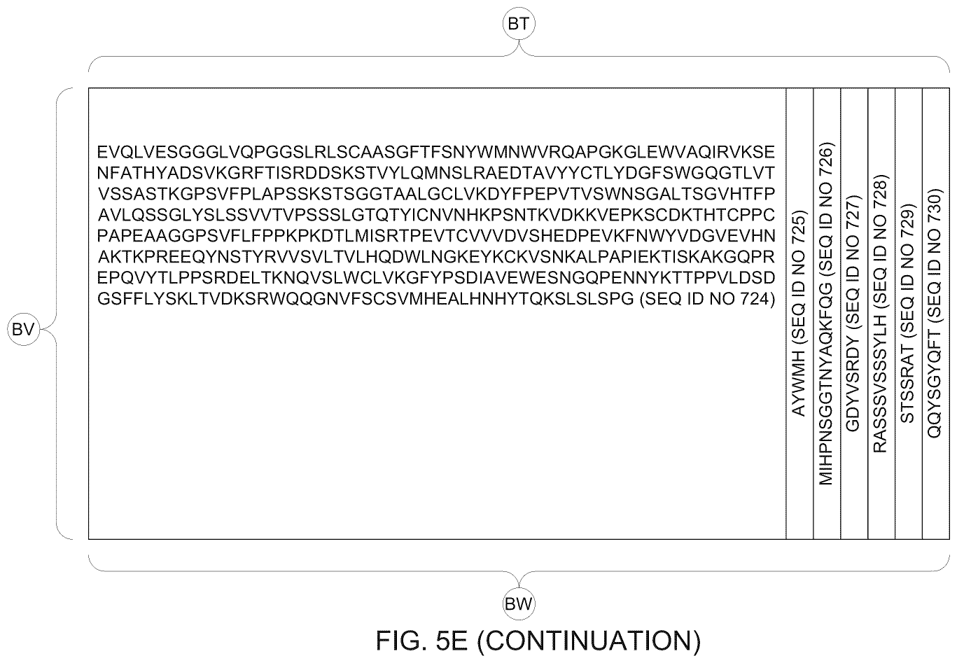

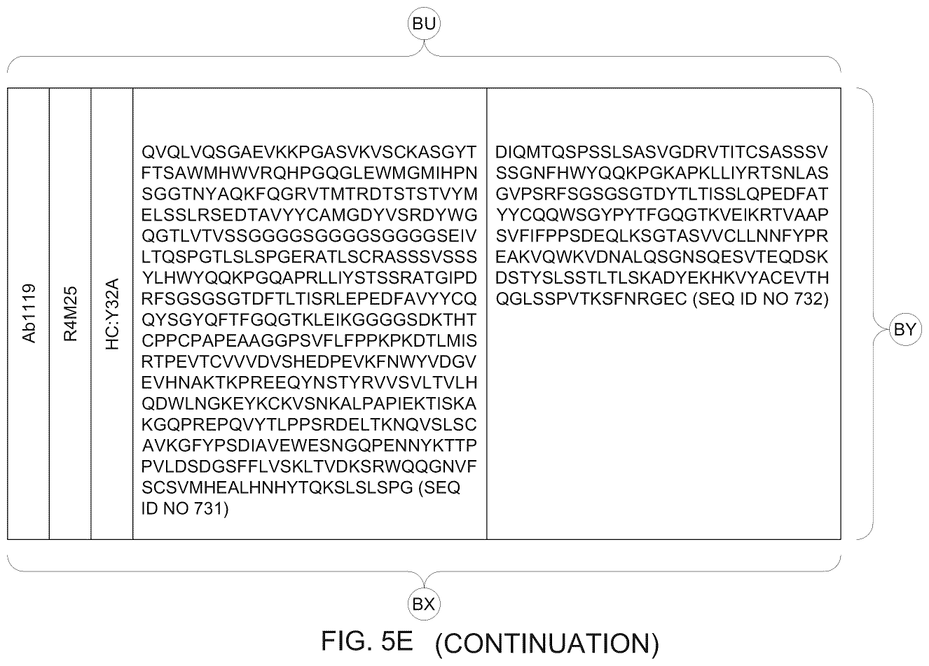

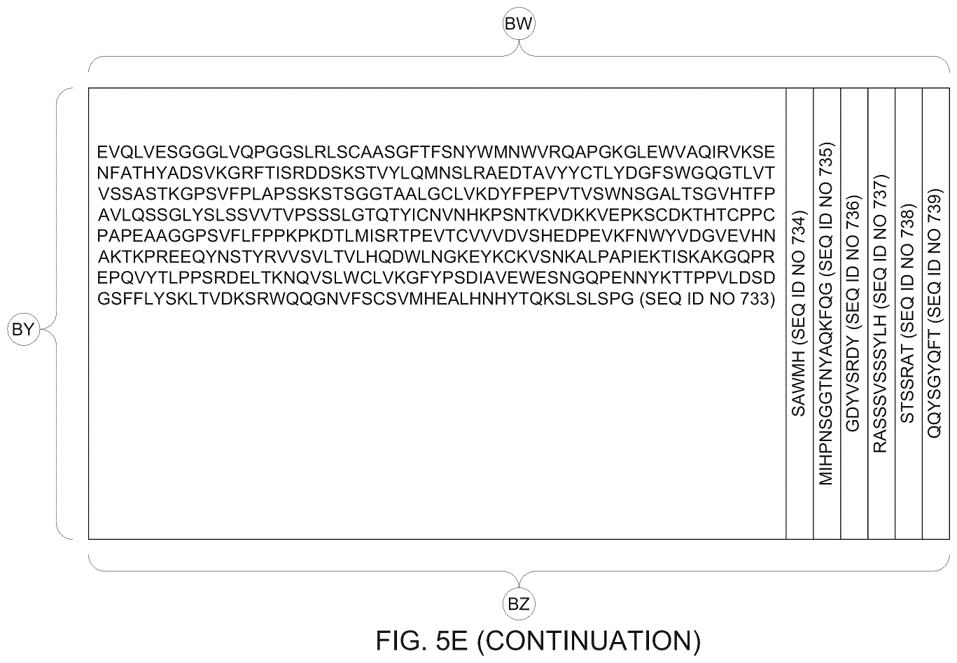

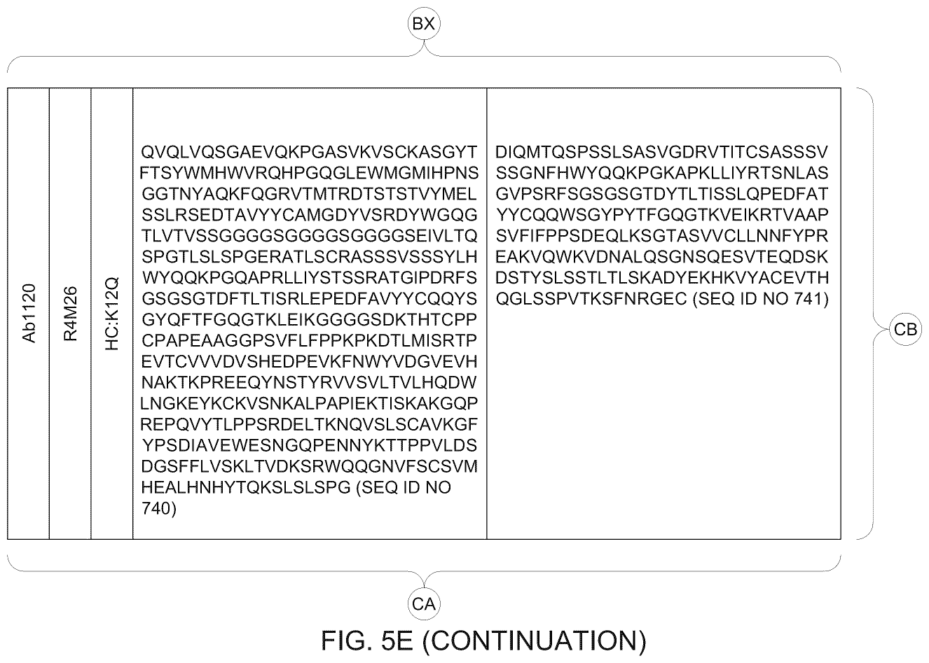

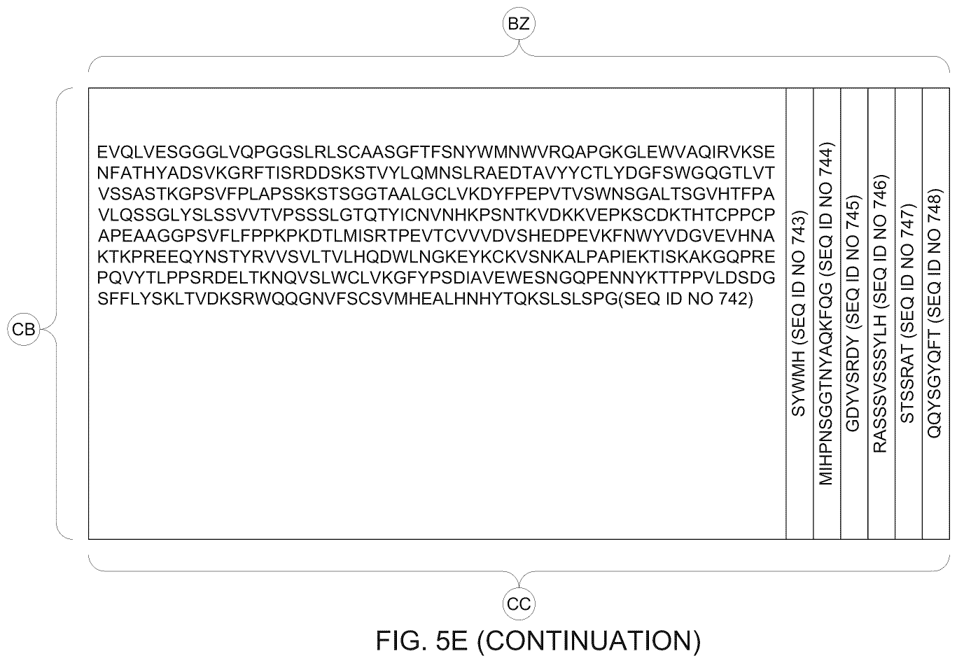

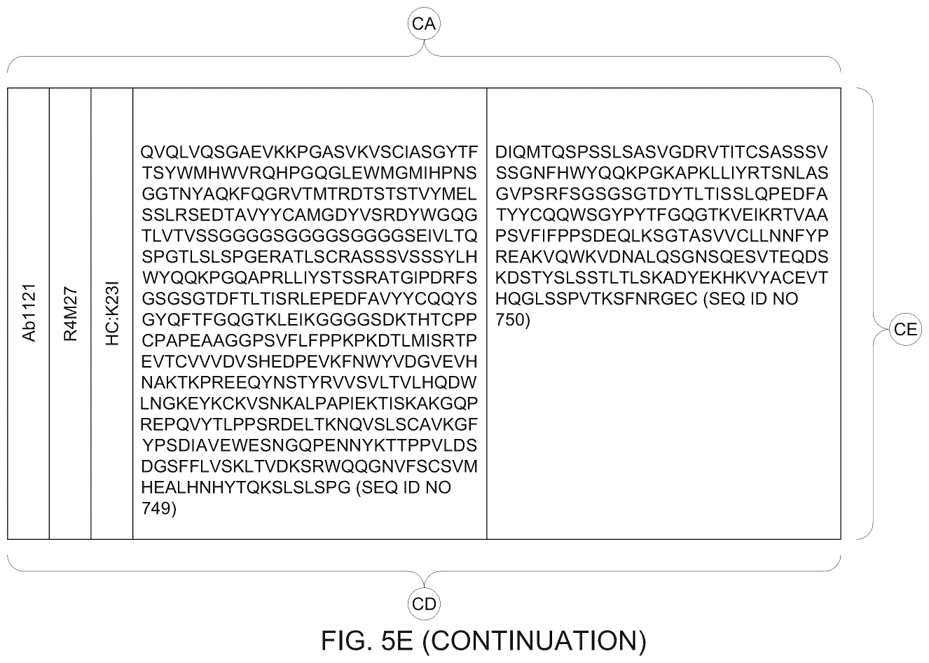

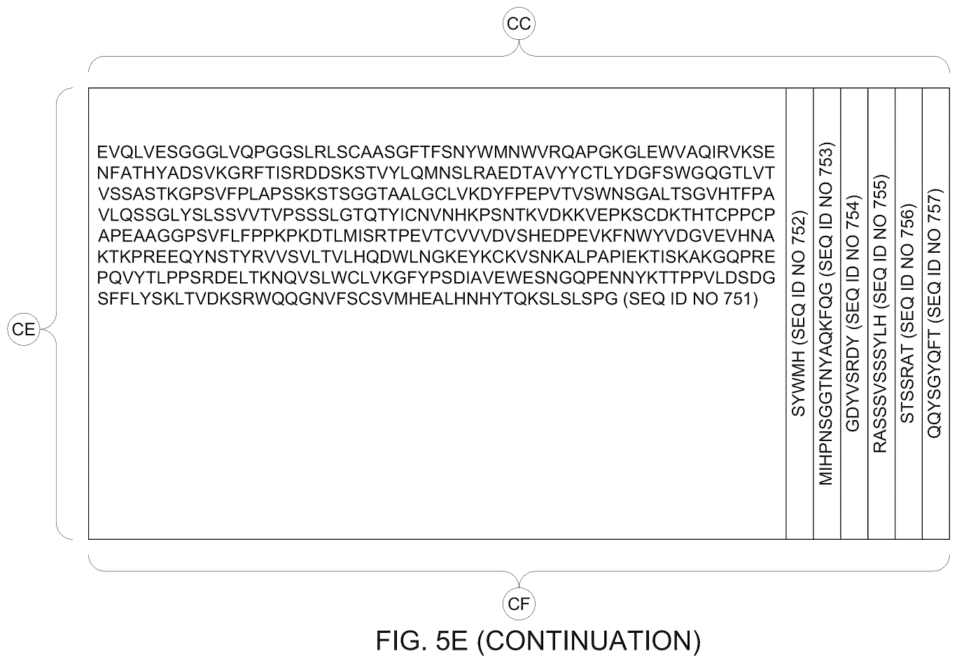

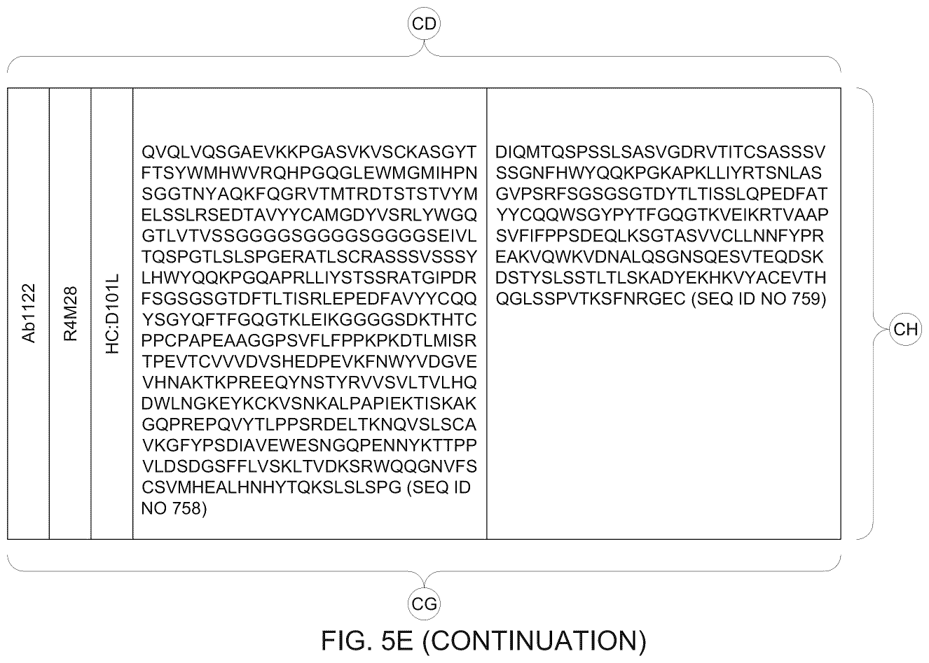

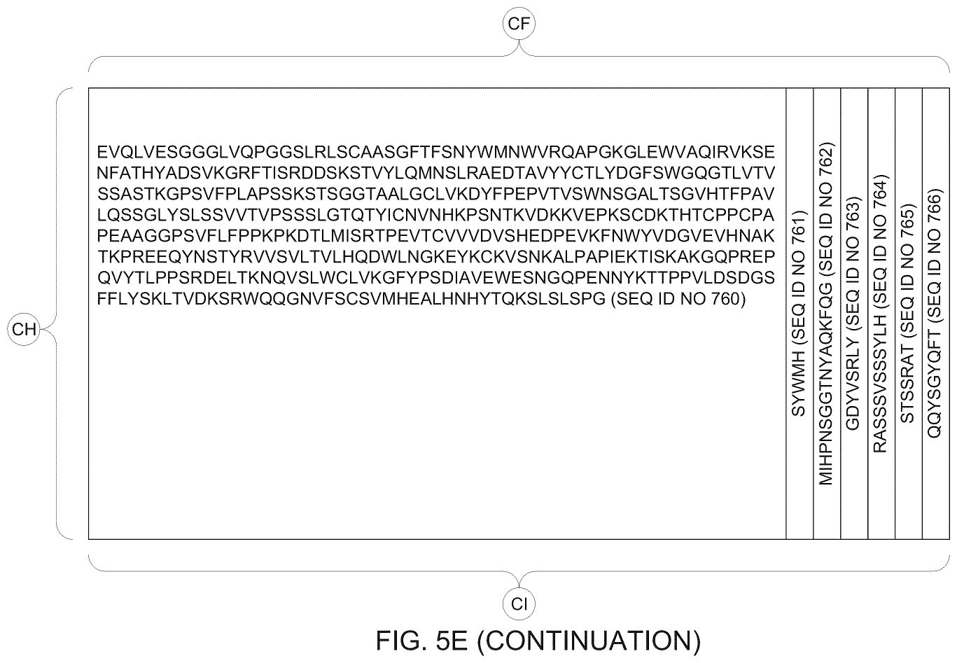

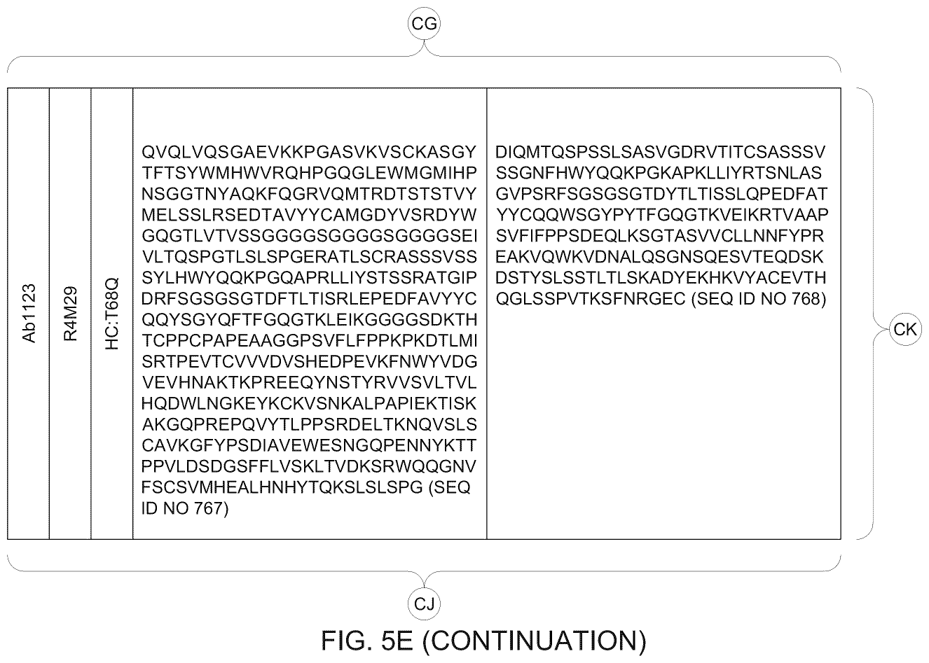

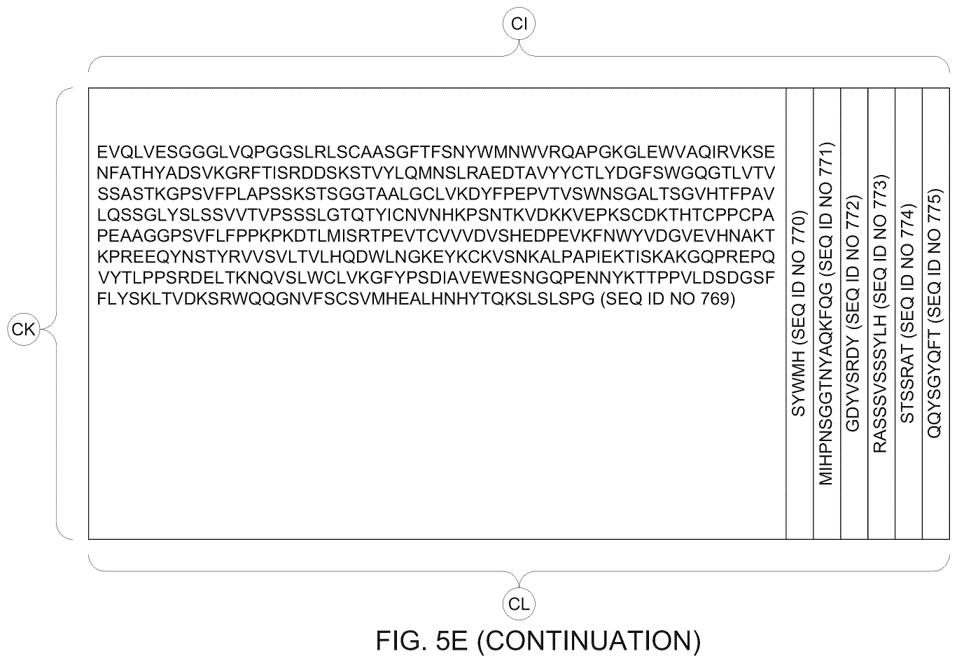

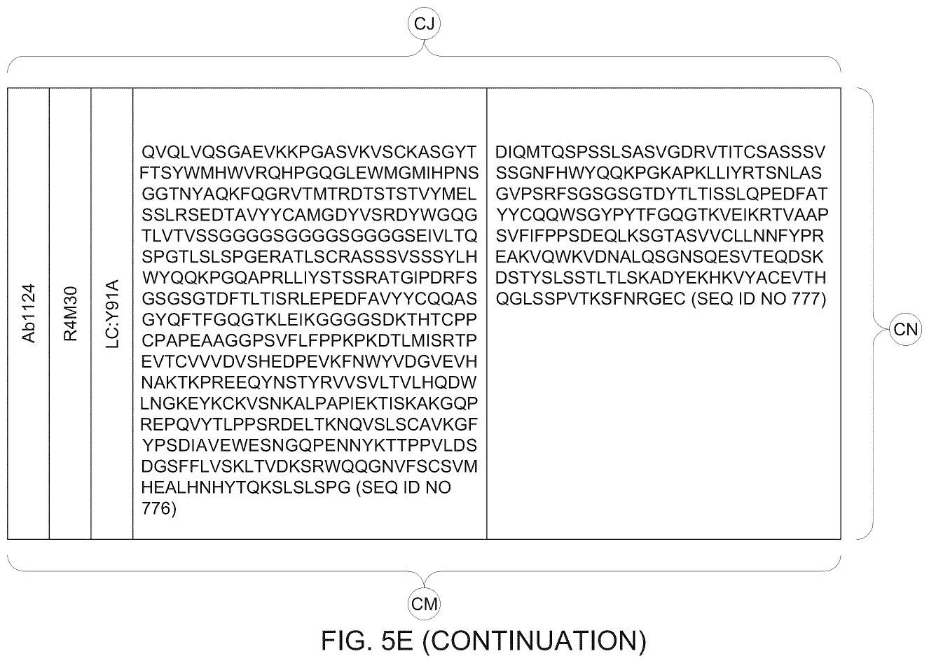

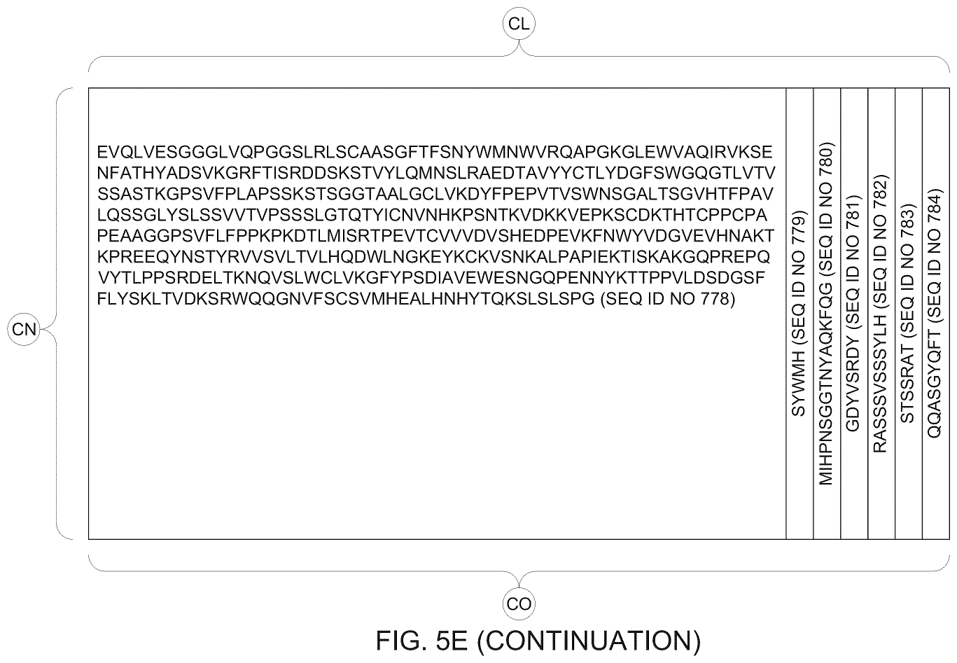

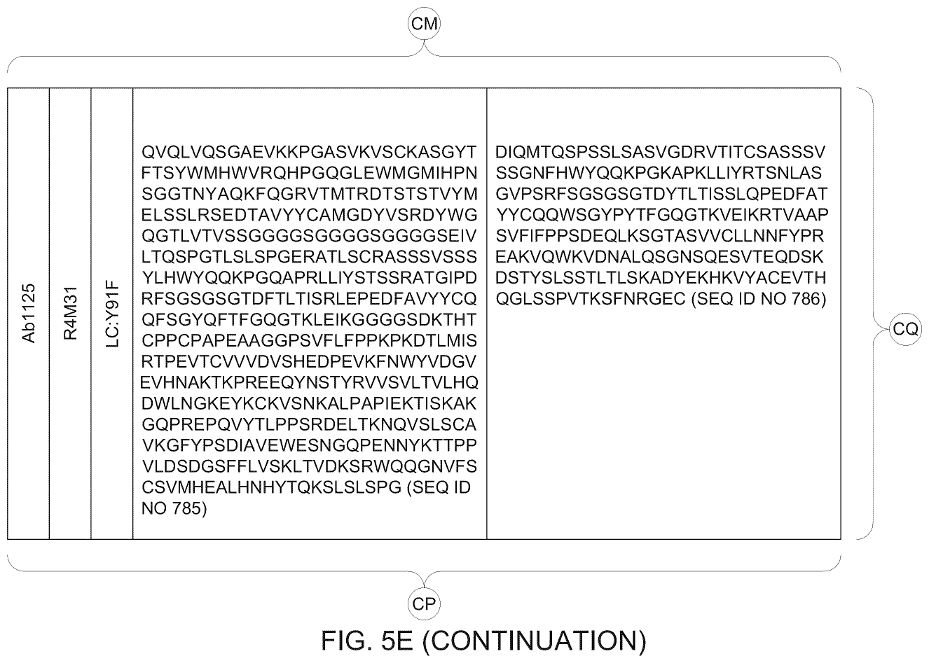

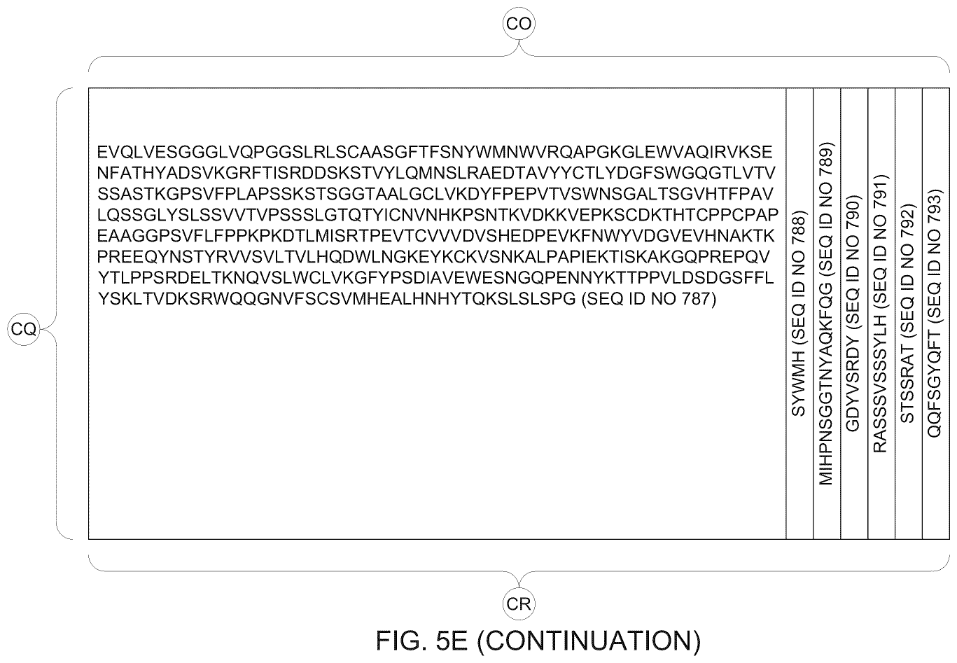

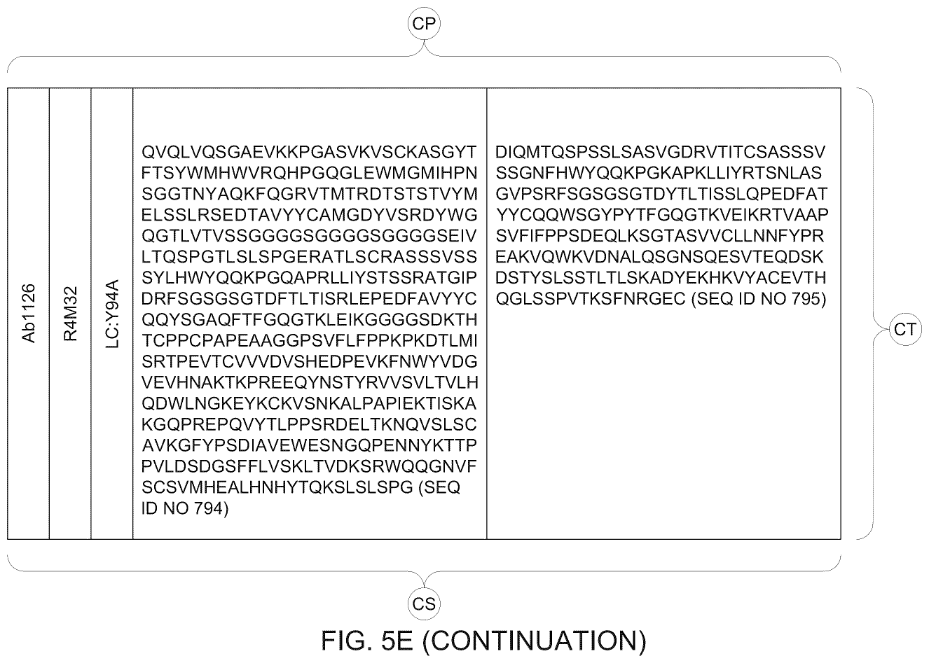

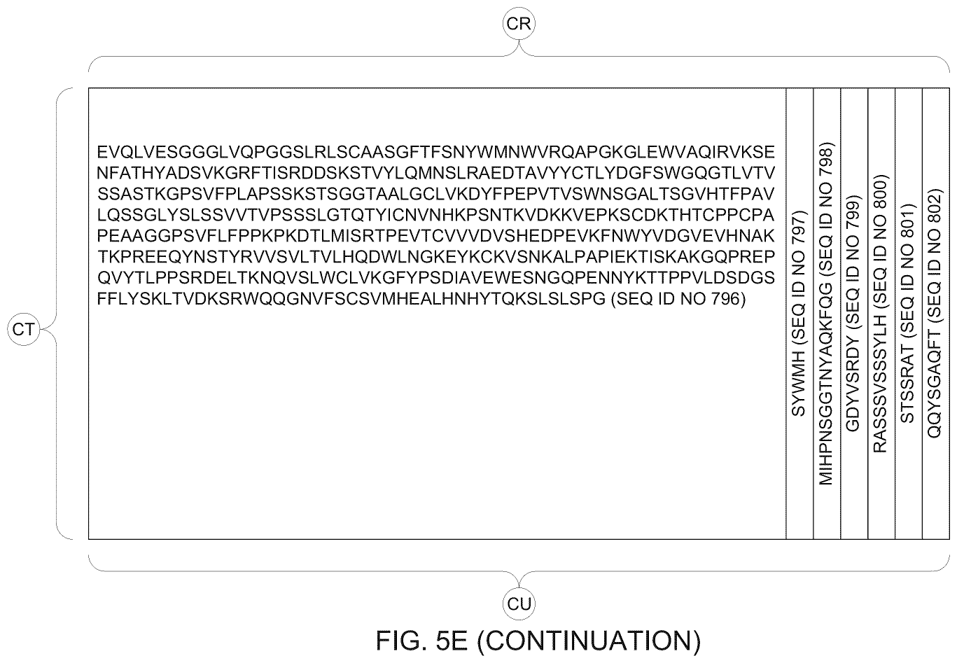

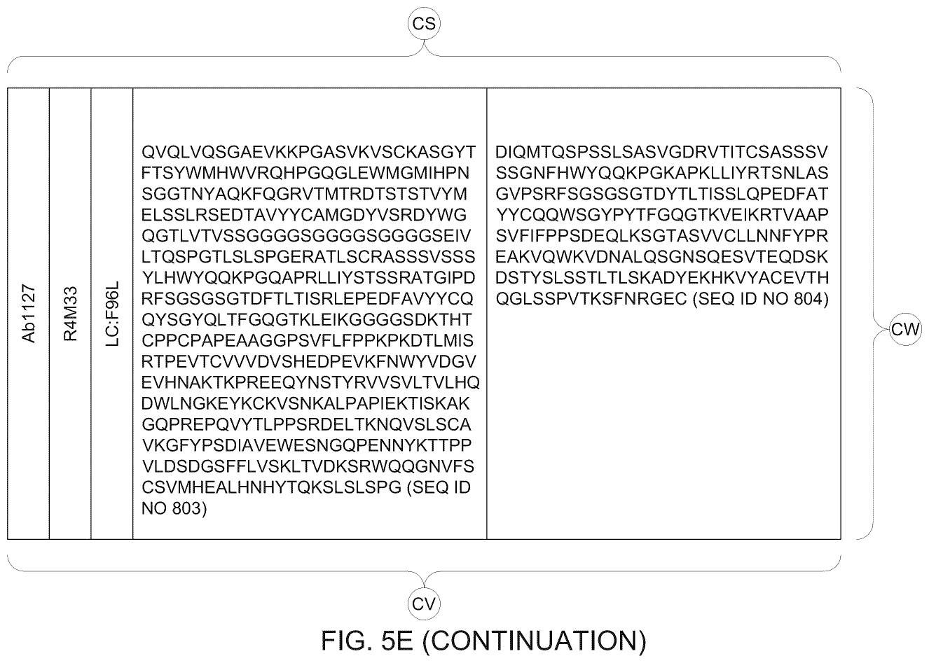

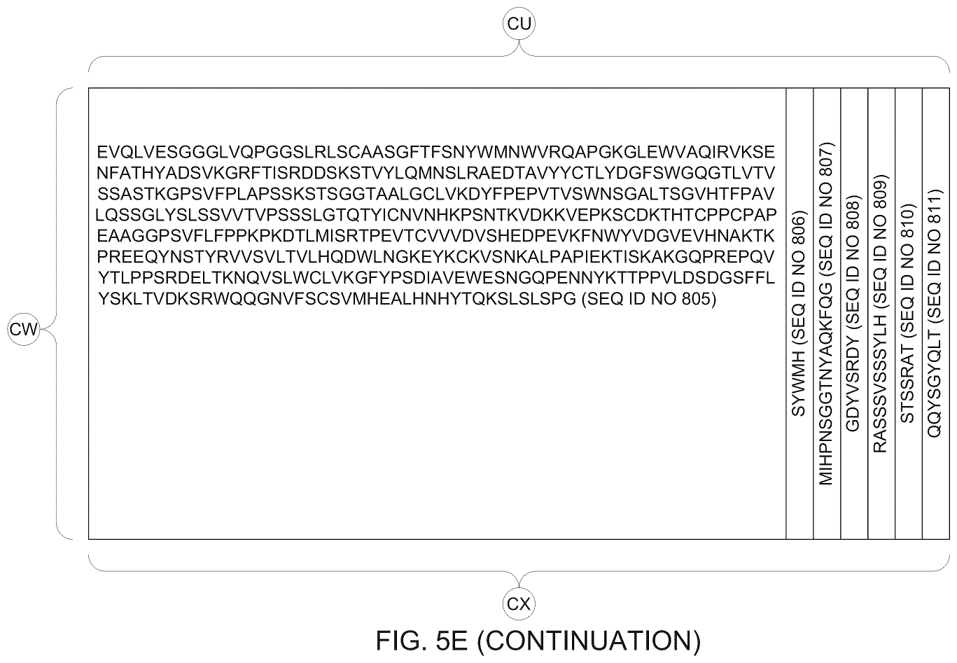

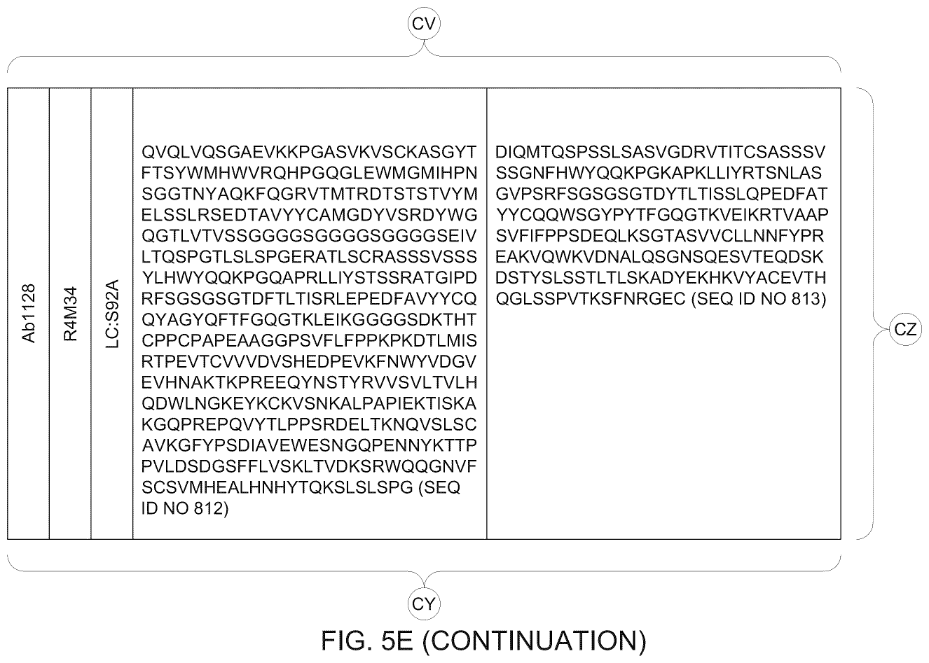

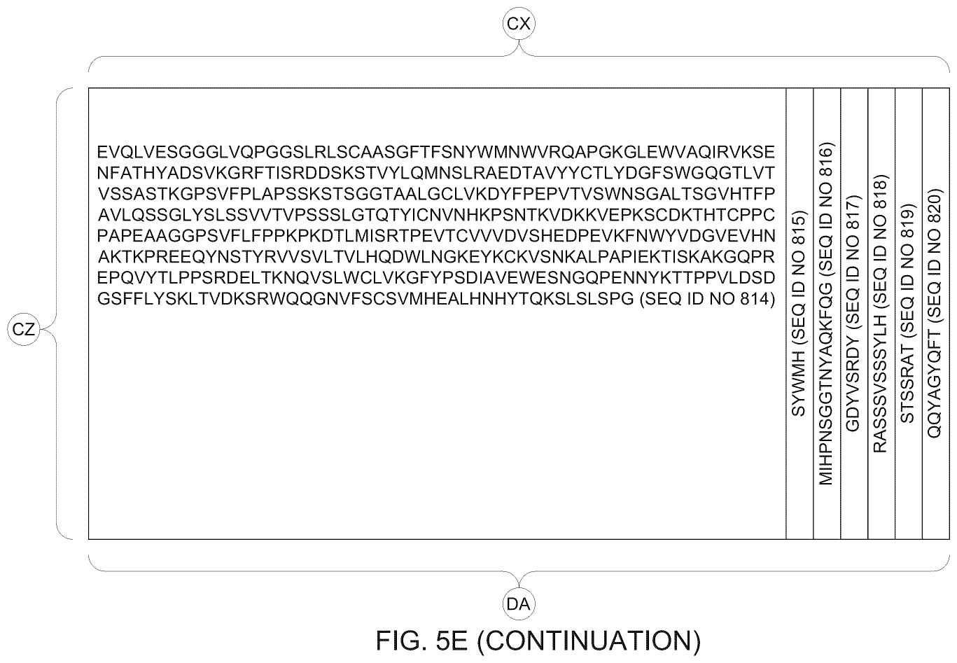

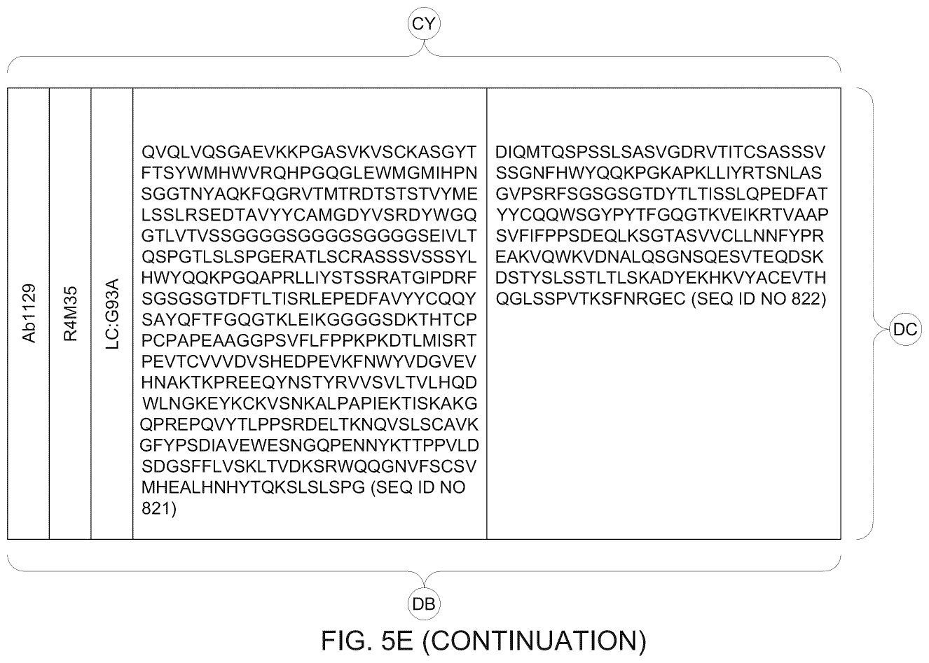

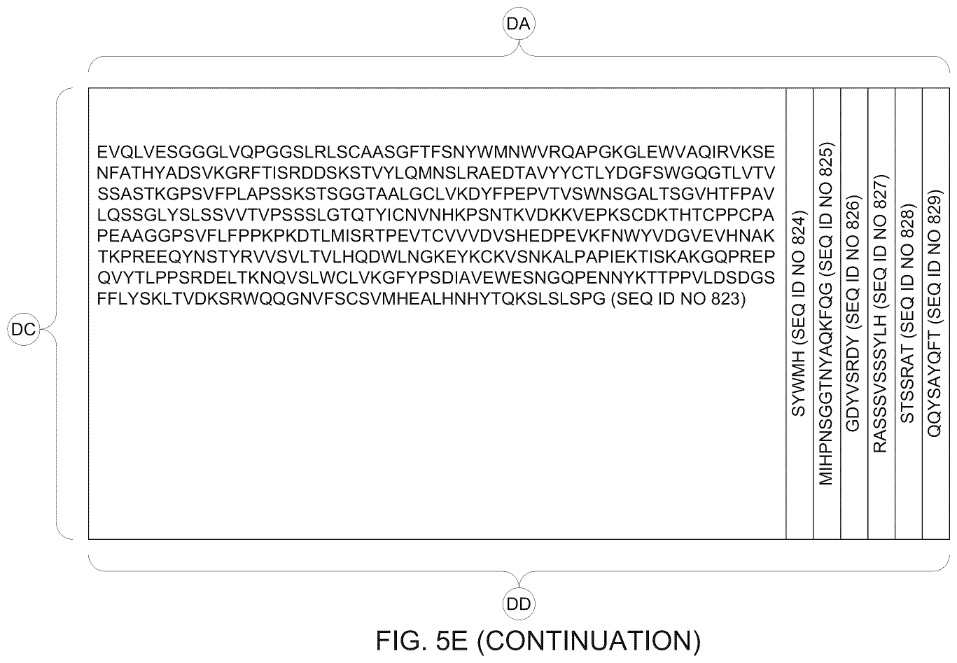

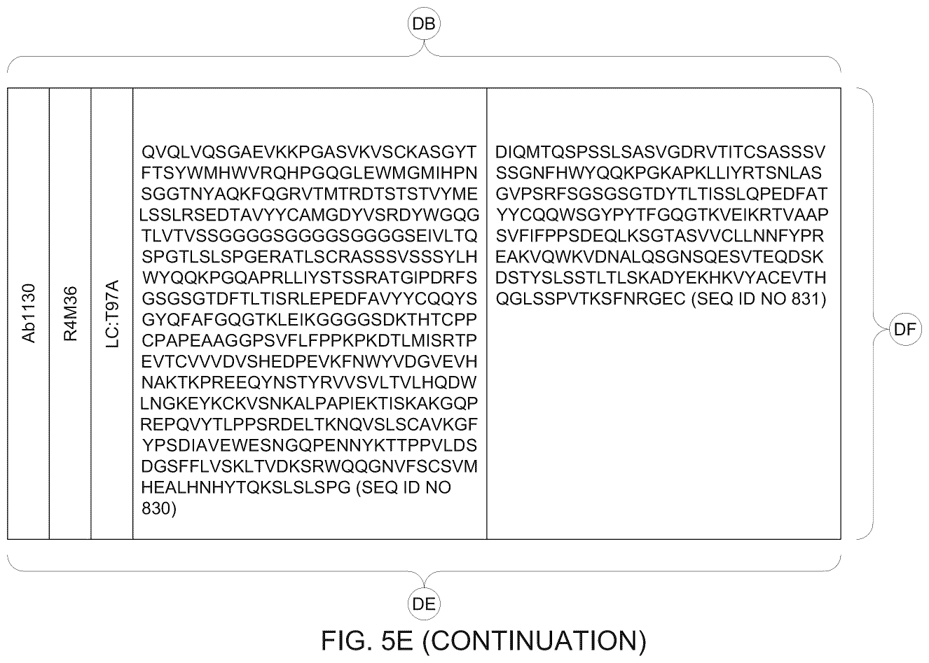

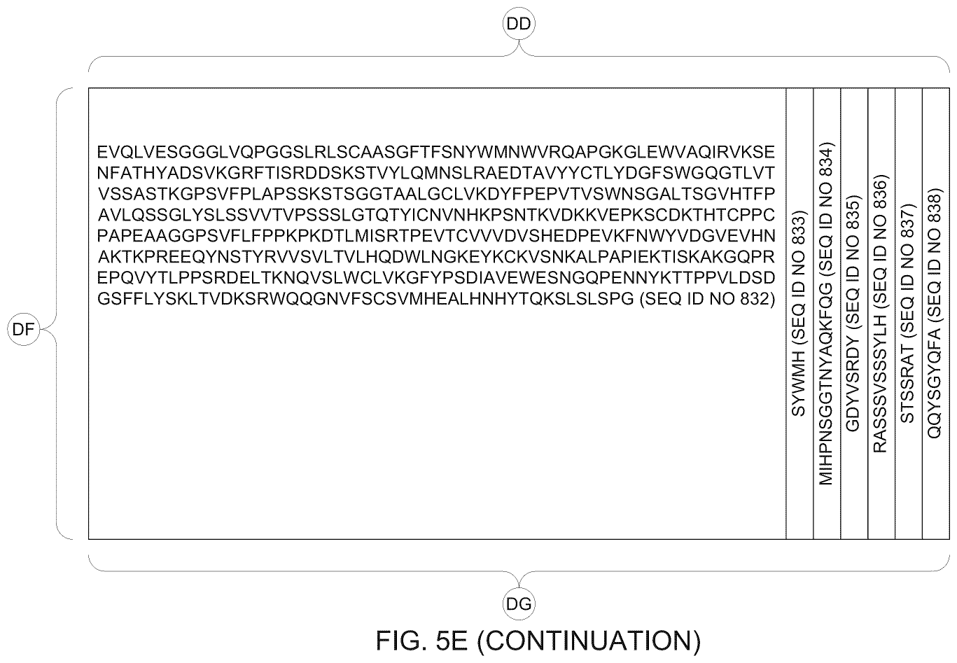

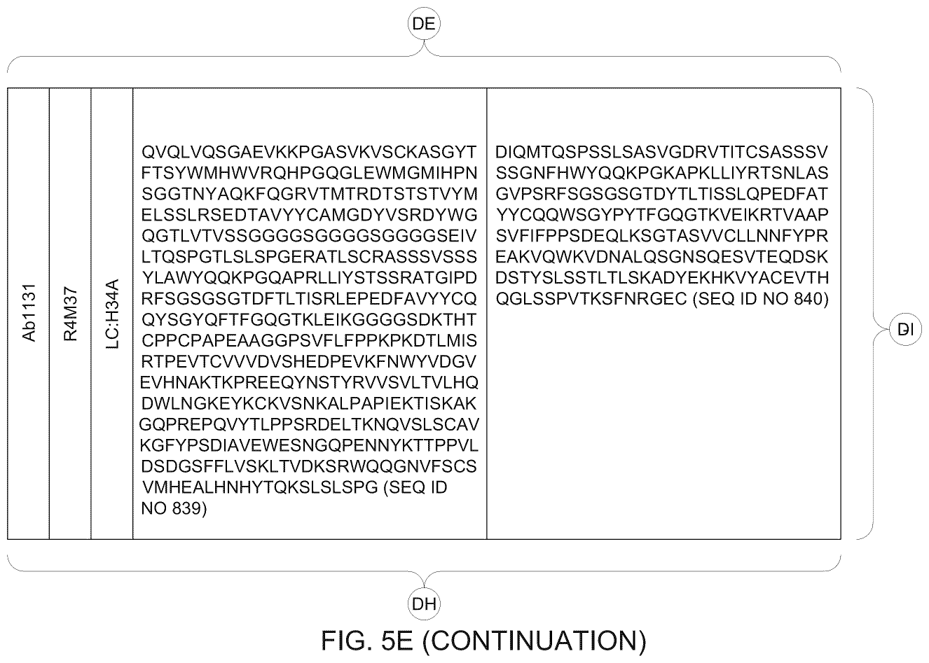

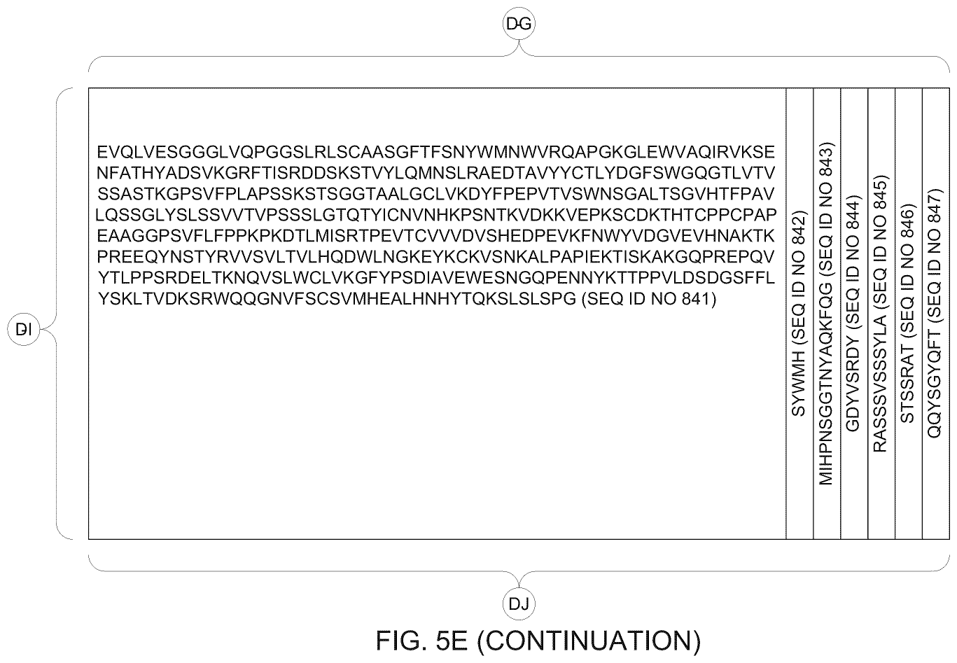

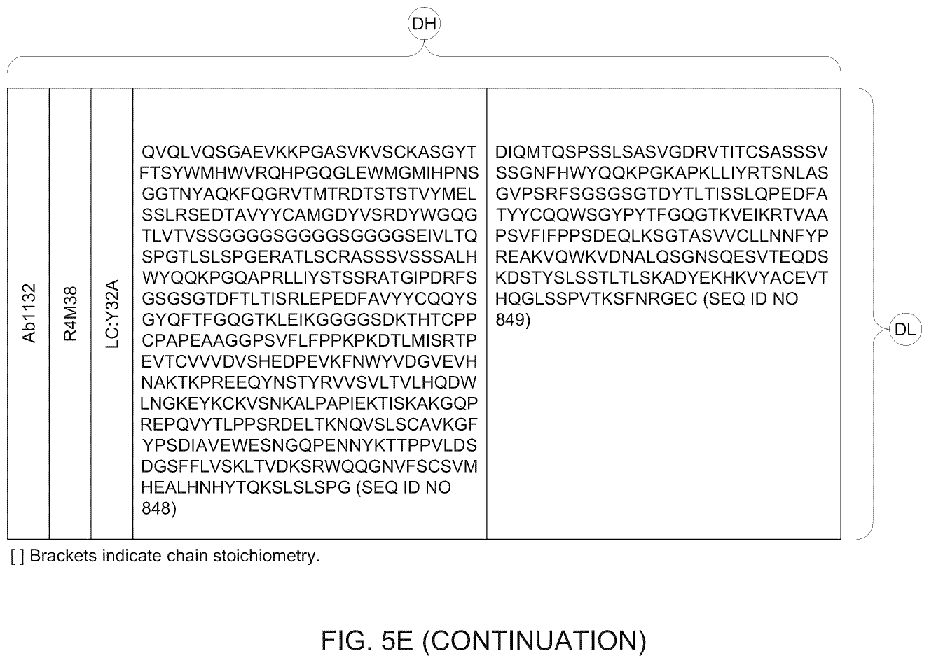

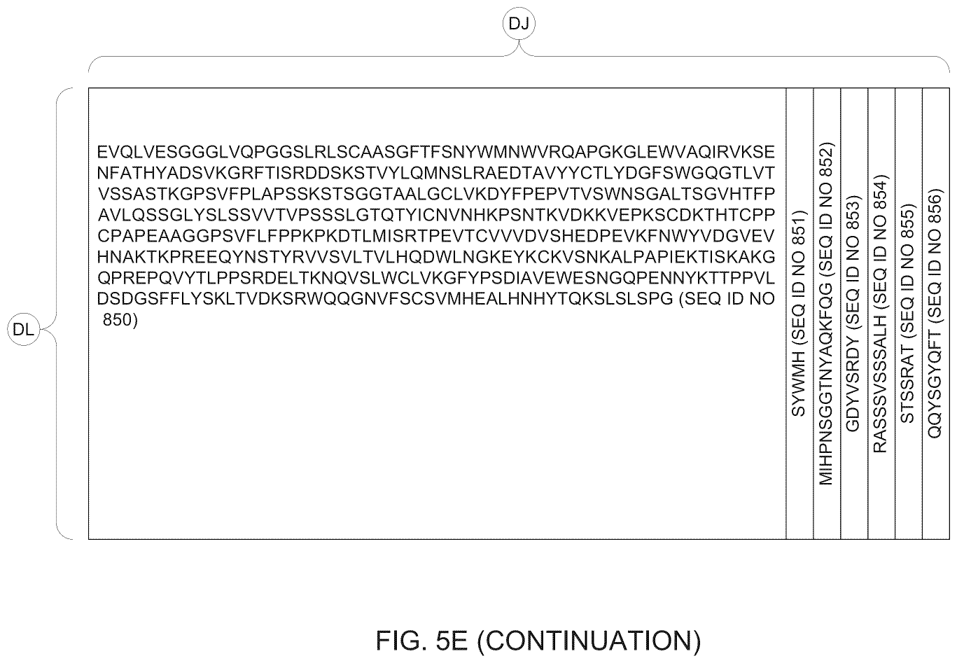

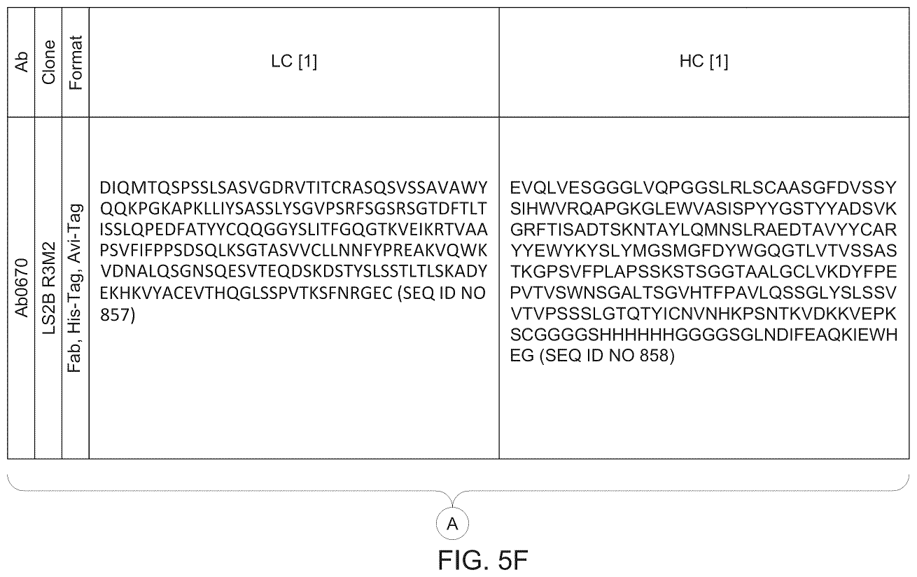

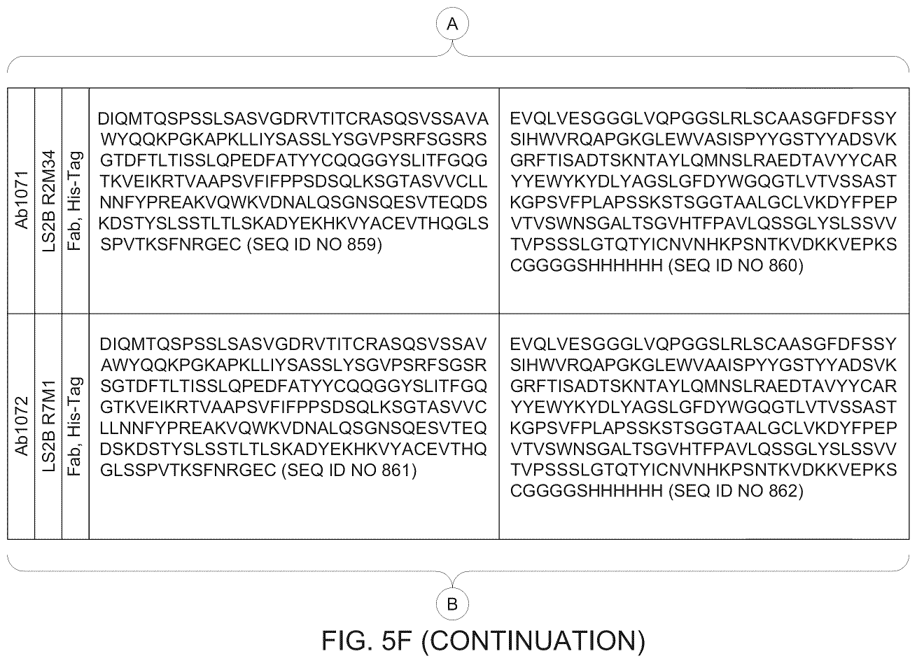

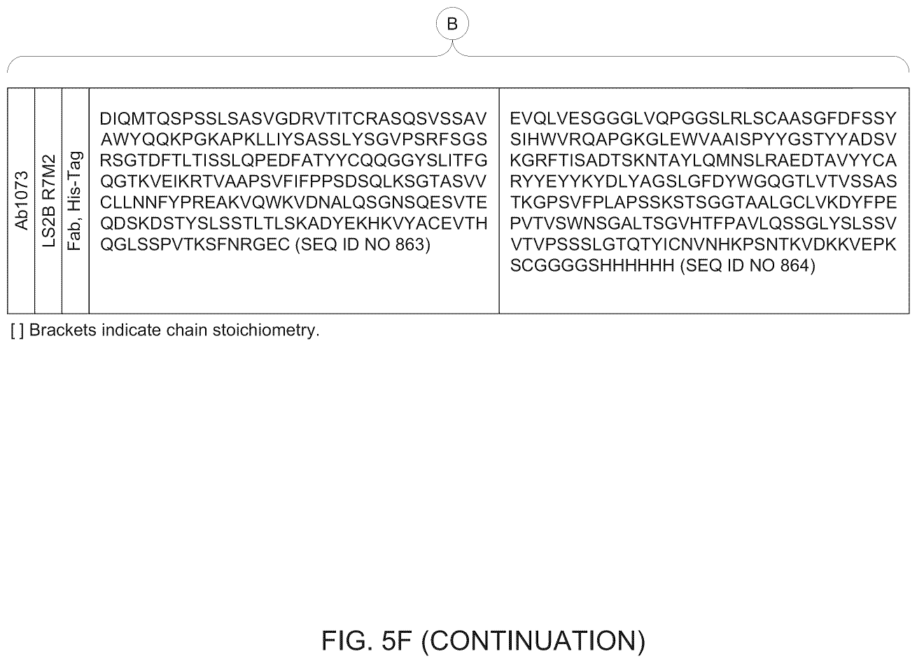

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 5A-5E optionally with one or more mutations. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 5A-5E optionally with one or more mutations at positions corresponding to one or more of positions 12, 23, 31, 33, 35, 51, 52, 53, 54, 55, 56, 57, 68, 97, 98, 99, 100, 102 in a heavy chain (HC) domain and positions 32, 34, 91, 92, 93, 94 and 97 in a light chain (LC) domain of Ab1094. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 5A-5E. In some embodiments, the vhCDR1, vhCDR2 and vhCDR3 sequences and said vlCDR1, vlCDR2, and vlCDR3 sequences have said one, two, three, four, five, fix or seven mutations described herein.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from Ab0220, Ab0617, Ab0618, Ab0658, Ab0660, Ab0661, Ab0662, Ab0663, Ab0666, Ab0667, Ab0726, Ab0727, Ab0728, Ab0729, Ab0730, Ab0731, Ab0732, Ab0733, Ab0734, Ab0735, Ab0736, Ab0738, Ab0739, Ab0740, Ab0741, Ab0742, Ab0745, Ab0746, Ab0747, Ab0748, Ab0749, Ab0750, Ab0751, Ab0753, Ab0755, Ab0756, Ab0757, Ab0758, Ab0759, Ab0760, Ab0761, Ab0762, Ab0763, Ab0785, Ab0786, Ab0787, Ab0788, Ab0898, Ab0899, Ab0900, Ab1094, Ab1095, Ab1096, Ab1097, Ab1098, Ab1099, Ab1102, Ab1103 Ab1104, Ab1105, Ab1106, Ab1107, Ab1108, Ab1109, Ab1110, Ab1111, Ab1114, Ab1115, Ab1117, Ab1118, Ab120, Ab121, Ab1123, Ab1124, Ab1126, Ab1128, Ab1129, Ab1130, Ab1131, and Ab1132 from FIGS. 5A-5E. In some embodiments, the sequences are from Ab0220. In some embodiments, the sequences are from Ab0617. In some embodiments, the sequences are from Ab0618. In some embodiments, the sequences are from Ab0658. In some embodiments, the sequences are from Ab0660. In some embodiments, the sequences are from Ab0661. In some embodiments, the sequences are from Ab0662. In some embodiments, the sequences are from Ab0663. In some embodiments, the sequences are from Ab0666. In some embodiments, the sequences are from Ab0667. In some embodiments, the sequences are from Ab0726. In some embodiments, the sequences are from Ab0727. In some embodiments, the sequences are from Ab0728. In some embodiments, the sequences are from Ab0729. In some embodiments, the sequences are from Ab0730. In some embodiments, the sequences are from Ab0731. In some embodiments, the sequences are from Ab0732. In some embodiments, the sequences are from Ab0733. In some embodiments, the sequences are from Ab0734. In some embodiments, the sequences are from Ab0735. In some embodiments, the sequences are from Ab0736. In some embodiments, the sequences are from Ab0738. In some embodiments, the sequences are from Ab0739. In some embodiments, the sequences are from Ab0740. In some embodiments, the sequences are from Ab0741. In some embodiments, the sequences are from Ab0742. In some embodiments, the sequences are from Ab0745. In some embodiments, the sequences are from Ab0746. In some embodiments, the sequences are from Ab0747. In some embodiments, the sequences are from Ab0748. In some embodiments, the sequences are from Ab0749. In some embodiments, the sequences are from Ab0750. In some embodiments, the sequences are from Ab0751. In some embodiments, the sequences are from Ab0753. In some embodiments, the sequences are from Ab0755. In some embodiments, the sequences are from Ab0756. In some embodiments, the sequences are from Ab0757. In some embodiments, the sequences are from Ab0758. In some embodiments, the sequences are from Ab0759. In some embodiments, the sequences are from Ab0760. In some embodiments, the sequences are from Ab0761. In some embodiments, the sequences are from Ab0762. In some embodiments, the sequences are from Ab0763. In some embodiments, the sequences are from Ab0785. In some embodiments, the sequences are from Ab0786. In some embodiments, the sequences are from Ab0787. In some embodiments, the sequences are from Ab0788. In some embodiments, the sequences are from Ab0898. In some embodiments, the sequences are from Ab0899. In some embodiments, the sequences are from Ab0900. In some embodiments, the sequences are from Ab1094. In some embodiments, the sequences are from Ab1095. In some embodiments, the sequences are from Ab1096. In some embodiments, the sequences are from Ab1097. In some embodiments, the sequences are from Ab1098. In some embodiments, the sequences are from Ab1099. In some embodiments, the sequences are from Ab1102. In some embodiments, the sequences are from Ab1103. In some embodiments, the sequences are from Ab1104. In some embodiments, the sequences are from Ab1105. In some embodiments, the sequences are from Ab1106. In some embodiments, the sequences are from Ab1107. In some embodiments, the sequences are from Ab1108. In some embodiments, the sequences are from Ab1109. In some embodiments, the sequences are from Ab1110. In some embodiments, the sequences are from Ab1111. In some embodiments, the sequences are from Ab1114. In some embodiments, the sequences are from Ab1115. In some embodiments, the sequences are from Ab1117. In some embodiments, the sequences are from Ab1118. In some embodiments, the sequences are from Ab1120. In some embodiments, the sequences are from Ab1121. In some embodiments, the sequences are from Ab1123. In some embodiments, the sequences are from Ab1124. In some embodiments, the sequences are from Ab1126. In some embodiments, the sequences are from Ab1128. In some embodiments, the sequences are from Ab1129. In some embodiments, the sequences are from Ab1130. In some embodiments, the sequences are from Ab1131. In some embodiments, the sequences are from Ab1132.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1094 optionally with one or more mutations at positions corresponding to one or more of positions 12, 23, 31, 33, 35, 51, 52, 53, 54, 55, 56, 57, 68, 97, 98, 99, 100, 102 in a heavy chain (HC) domain and positions 32, 34, 91, 92, 93, 94 and 97 in a light chain (LC) domain. In some embodiments, one or more of the vhCDR1, vhCDR2 and vhCDR3 sequences and the vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1094.

In some embodiments, the indinavir binding domain may comprise a VH domain and VL domain selected from the group consisting of the VH and VL domains from FIGS. 5A-5E optionally with one or more mutations. In some embodiments, the indinavir binding domain may comprise a VH domain and VL domain selected from the group consisting of the VH and VL domains from Figures SA-SE. In some embodiments, the indinavir binding domain may comprise a sequence having at least 85%, 86%, 87%, 88%, 89%, 90%, 91%, 92%, 93%, 94%, 95%, 96%, 97%, 98%, 99%, 100% sequence identity with the VH domain sequence of the Ab1094 and/or a sequence having at least 85%, 86%, 87%, 88%, 89%, 90%, 91%, 92%, 93%, 94%, 95%, 96%, 97%, 98%, 99%, 100% sequence identity with the VL domain sequence of the Ab1094. In some embodiments, the indinavir binding domain may comprise the VH and VL domain sequences of the Ab1094. In some embodiments, the indinavir binding domain may comprise the Ab1094.

In some embodiments, the vhCDR1, vhCDR2 and vhCDR3 sequences and said vlCDR1, vlCDR2, and vlCDR3 sequences may comprise one or more mutations selected from the group consisting of mutations corresponding to HC G44C+LC Q100C, LC Y94D, LC L95A, LC L95D, LC L95K, LC L95Q, HC T73K, and HC V78A of the Ab0220. In some embodiments, the vhCDR1, vhCDR2 and vhCDR3 sequences and said vlCDR1, vlCDR2, and vlCDR3 sequences may comprise one or more mutations selected from the group consisting of mutations corresponding to HC R71V+LC L95Q, HC M48I+V67A+M69L+R71V+T73K and LCL95Q, HC A40R+LC L95Q, LC A43H+L95Q, and HC G44R+LC L95Q of the Ab0220. In some embodiments, the vhCDR1, vhCDR2 and vhCDR3 sequences and said vlCDR1, vlCDR2, and vlCDR3 sequences may comprise one or more mutations selected from the group consisting of mutations corresponding to I50M+S58N, 150M, and S58N in the HC domain of the Ab00785. In some embodiments, the vhCDR1, vhCDR2 and vhCDR3 sequences and said vlCDR1, vlCDR2, and vlCDR3 sequences may comprise one or more mutations selected from the group consisting of mutations corresponding to I50M+S58N and 150M in the HC domain of the Ab0898. In some embodiments, the vhCDR1, vhCDR2 and vhCDR3 sequences and said vlCDR1, vlCDR2, and vlCDR3 sequences may comprise one or more mutations selected from the group consisting of mutations corresponding to W33A, W33F, W33H, W33L, H35A, H52N, H52A, R100A, N53A, N53S, N53Q, S54A, I51A, G55A, G56S, T57A, Y8G95A, Y97A, V98A, S99A, Y102A, S31A, K12Q, K23I, and T68Q in the heavy chain (HC) domain and Y91A, Y94A, S92A, G93A, 197A, H34A, and Y32A in the light chain (LC) domain of Ab1094. In some embodiments, the sequences have a mutation corresponding to said W33A. In some embodiments, the sequences have a mutation corresponding to said W33F. In some embodiments, the sequences have a mutation corresponding to said W33H. In some embodiments, the sequences have a mutation corresponding to said W33L. In some embodiments, the sequences have a mutation corresponding to said H35A. In some embodiments, the sequences have a mutation corresponding to said H52N. In some embodiments, the sequences have a mutation corresponding to said H52A. In some embodiments, the sequences have a mutation corresponding to said R100A. In some embodiments, the sequences have a mutation corresponding to said N53A. In some embodiments, the sequences have a mutation corresponding to said N53S. In some embodiments, the sequences have a mutation corresponding to said N53Q. In some embodiments, the sequences have a mutation corresponding to said S54A. In some embodiments, the sequences have a mutation corresponding to said I51A. In some embodiments, the sequences have a mutation corresponding to said G55A. In some embodiments, the sequences have a mutation corresponding to said G56S. In some embodiments, the sequences have a mutation corresponding to said T57A. In some embodiments, the sequences have a mutation corresponding to said Y8G95A. In some embodiments, the sequences have a mutation corresponding to said Y97A. In some embodiments, the sequences have a mutation corresponding to said V98A. In some embodiments, the sequences have a mutation corresponding to said S99A. In some embodiments, the sequences have a mutation corresponding to said Y102A. In some embodiments, the sequences have a mutation corresponding to said S31A. In some embodiments, the sequences have a mutation corresponding to said K12Q. In some embodiments, the sequences have a mutation corresponding to said K23I. In some embodiments, the sequences have a mutation corresponding to said T68Q. In some embodiments, the sequences have a mutation corresponding to said Y91A. In some embodiments, the sequences have a mutation corresponding to said Y94A. In some embodiments, the sequences have a mutation corresponding to said S92A. In some embodiments, the sequences have a mutation corresponding to said G93A. In some embodiments, the sequences have a mutation corresponding to said T97A. In some embodiments, the sequences have a mutation corresponding to said H34A. In some embodiments, the sequences have a mutation corresponding to said Y32A.

In some embodiments, the composition may be a fusion protein.

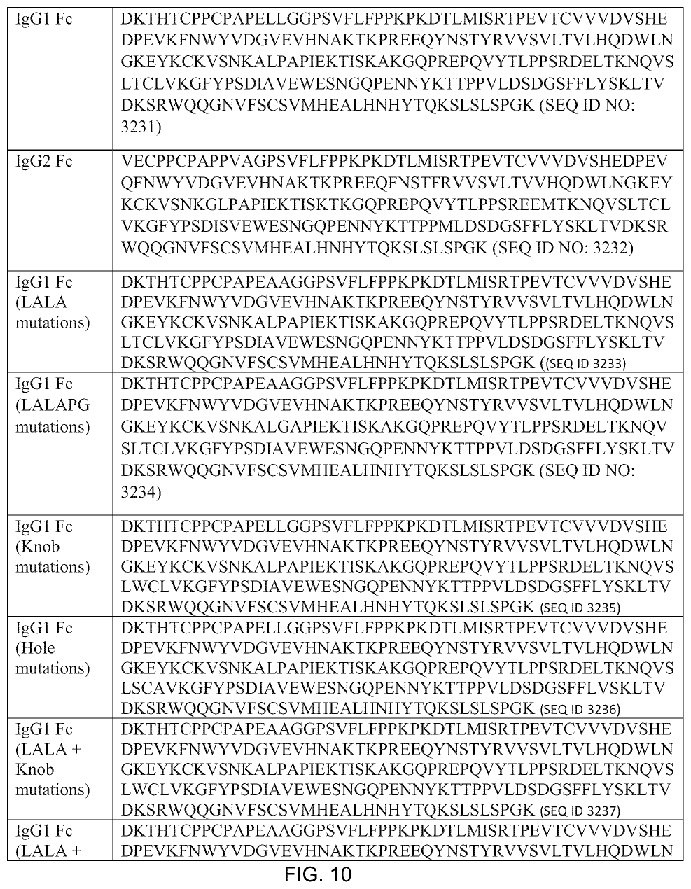

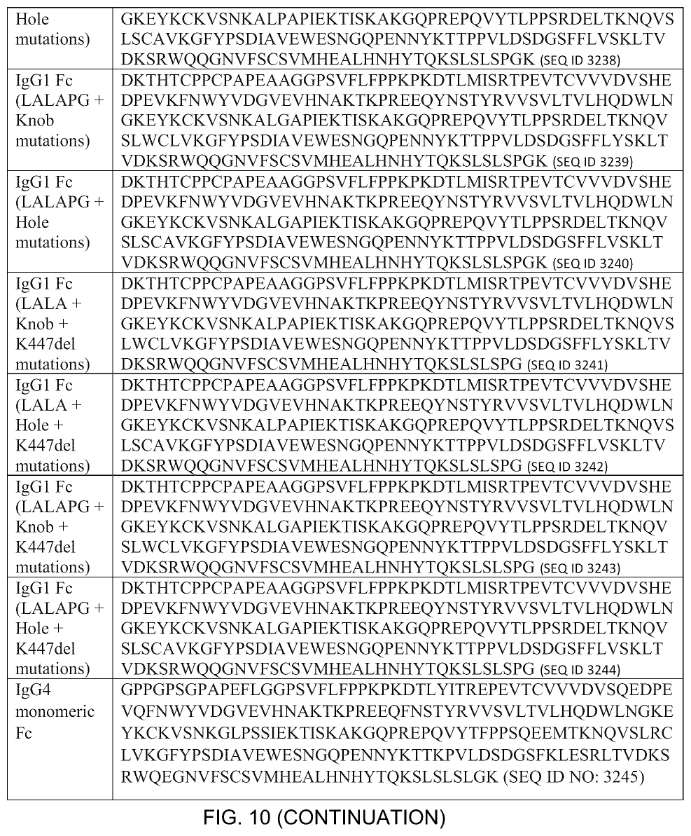

In some embodiments, said composition comprises an Fc domain with one or more knob variants. In some embodiments, said composition comprises an Fc domain with one or more hole variants. Some examples of the knob and hole Fc sequences are disclosed in FIG. 10. In some embodiments, the Fc domain described herein having a knob variant comprises a sequence having at least 85, 86, 87, 88, 89, 90, 91, 92, 93, 94, 95, 96, 97, 98, 99 and 100% identity to any one of SEQ ID NO: 3235, 3237, 3239, 3241, and 3243. In some embodiments, the Fc domain described herein having a hole variant comprises a sequence having at least 85, 86, 87, 88, 89, 90, 91, 92, 93, 94, 95, 96, 97, 98, 99 and 100% identity to any one of SEQ ID NO: 3236, 3238, 3240, 3242, and 3244. In some embodiments, the Fc domain described herein having a knob variant comprises the sequence of 3235. In some embodiments, the Fc domain described herein having a knob variant comprises the sequence of 3237. In some embodiments, the Fc domain described herein having a knob variant comprises the sequence of 3239. In some embodiments, the Fc domain described herein having a knob variant comprises the sequence of 3241. In some embodiments, the Fc domain described herein having a knob variant comprises the sequence of 3243. In some embodiments, the Fc domain described herein having a hole variant comprises the sequence of SEQ ID NO: 3236. In some embodiments, the Fc domain described herein having a hole variant comprises the sequence of SEQ ID NO: 3238. In some embodiments, the Fc domain described herein having a hole variant comprises the sequence of SEQ ID NO: 3240. In some embodiments, the Fc domain described herein having a hole variant comprises the sequence of SEQ ID NO: 3242. In some embodiments, the Fc domain described herein having a hole variant comprises the sequence of SEQ ID NO: 3244.

In some embodiments, the indinavir binding domain may comprise scFv means for binding indinavir. In some embodiments, the indinavir binding domain may comprise Fab means for binding indinavir.

In some embodiments, the composition may be a nucleic acid composition comprising a polynucleotide encoding a composition comprising an indinavir binding domain. In some embodiments, an expression vector composition comprising the nucleic acid composition. In some embodiments, a host cell may comprise the expression vector.

In some embodiments, the method of making the composition of the present disclosure may comprise an immunization campaign, validation of murine indinavir binders, humanization of the murine indinavir binders, generation of the composition and validation of the composition.

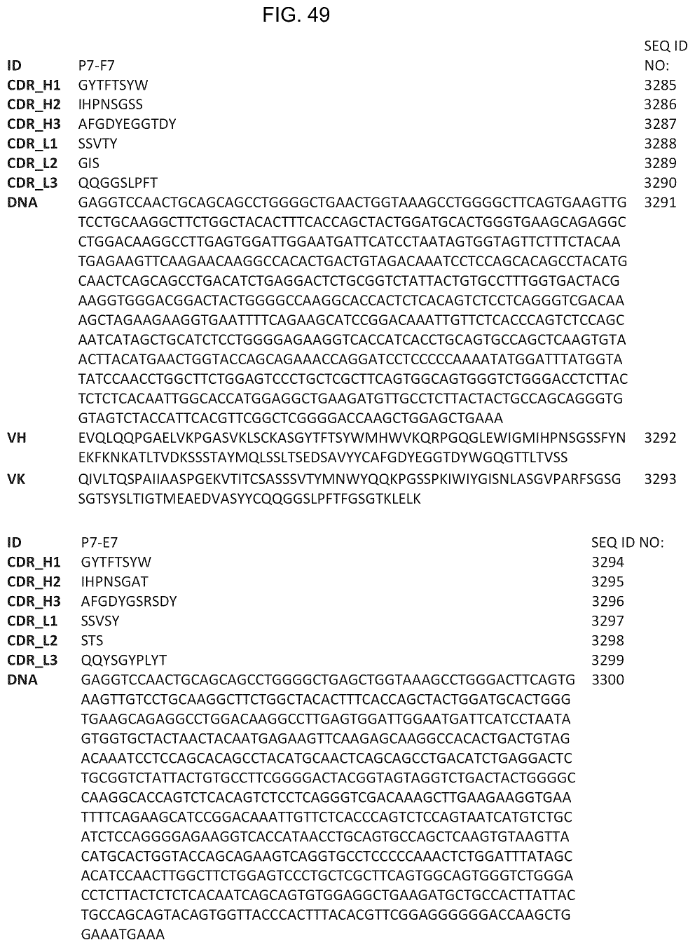

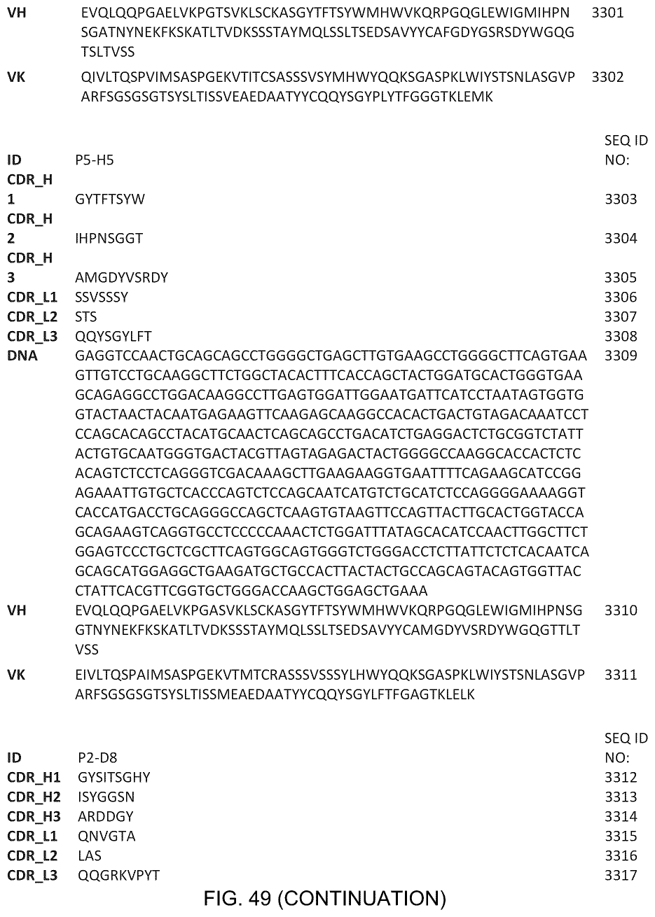

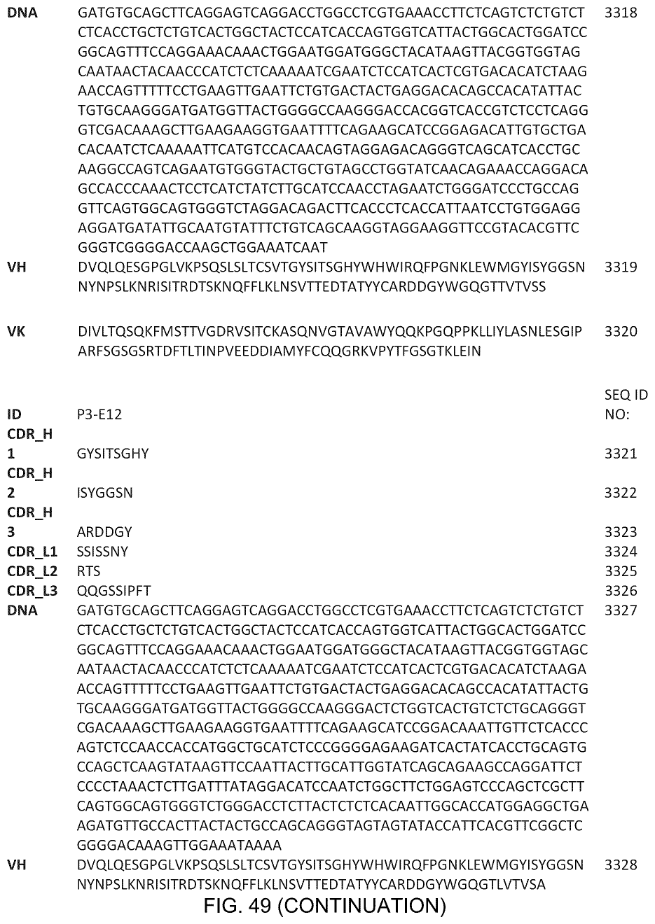

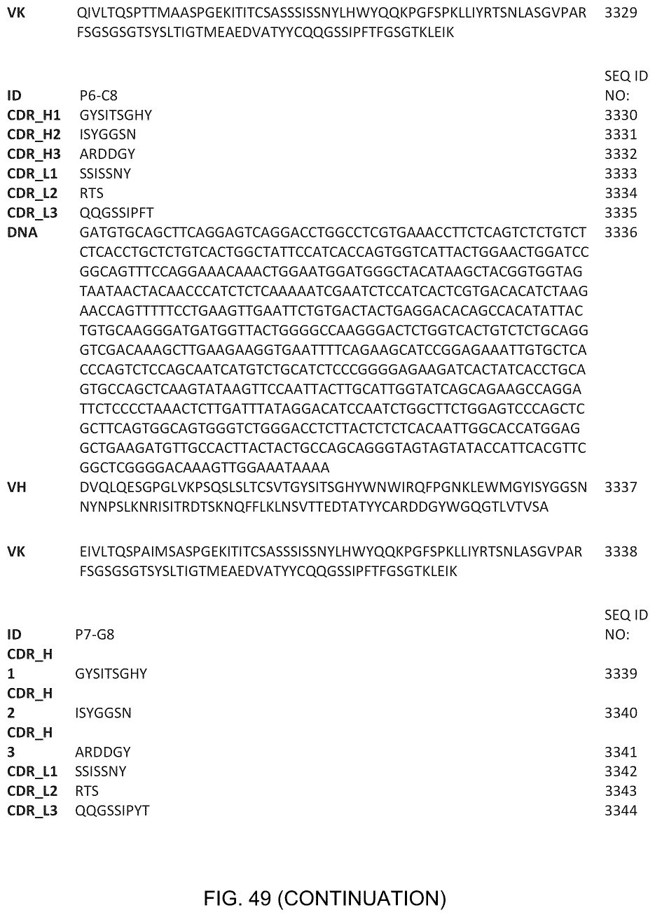

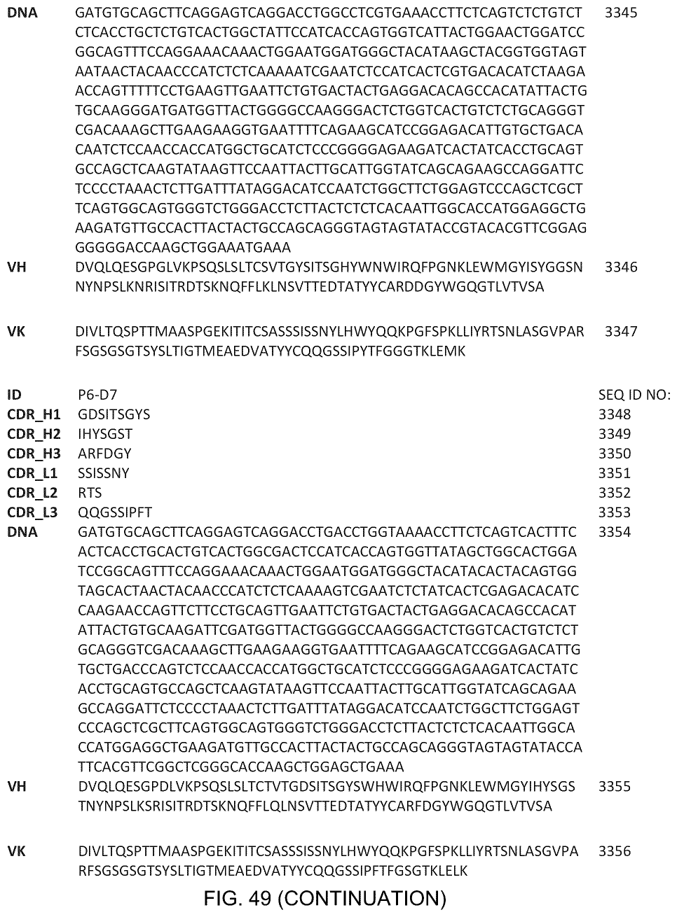

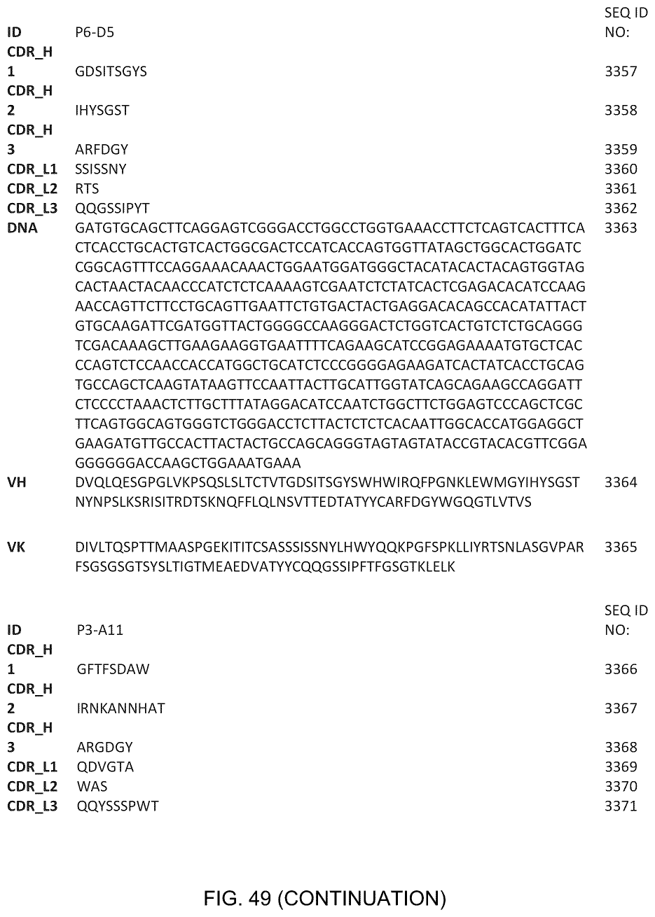

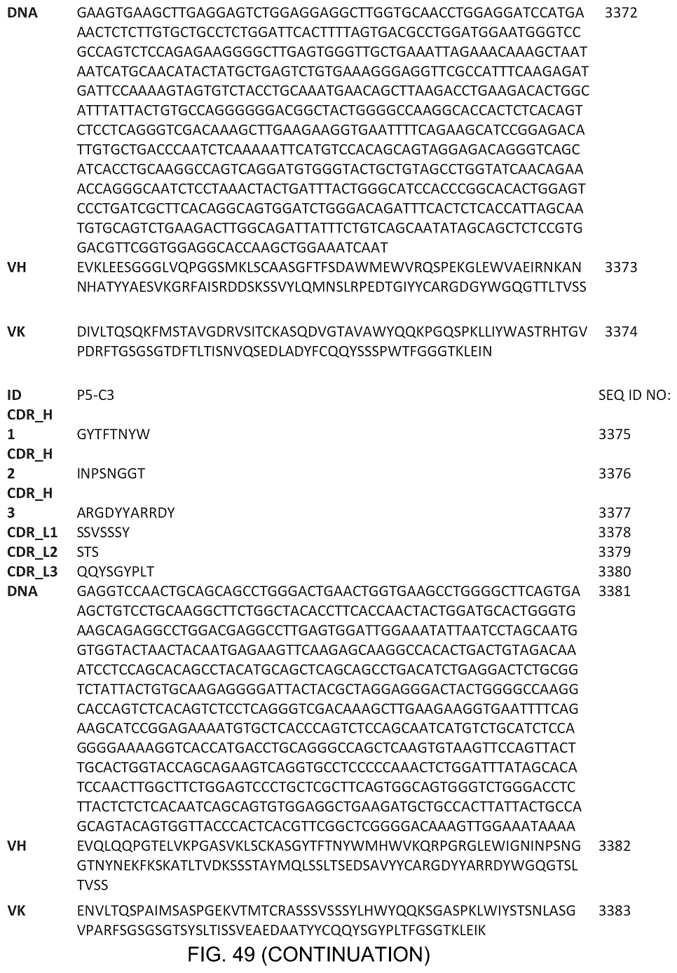

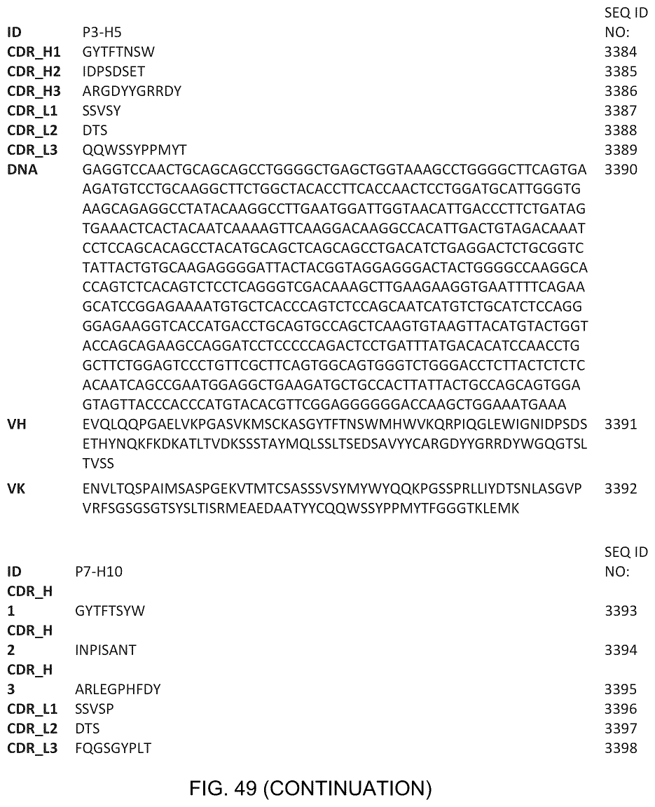

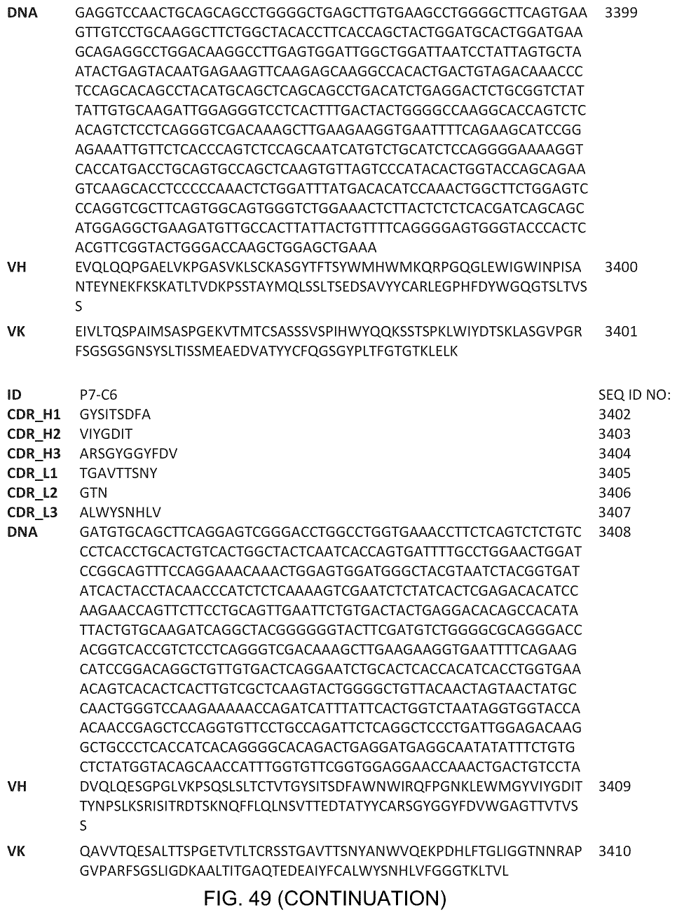

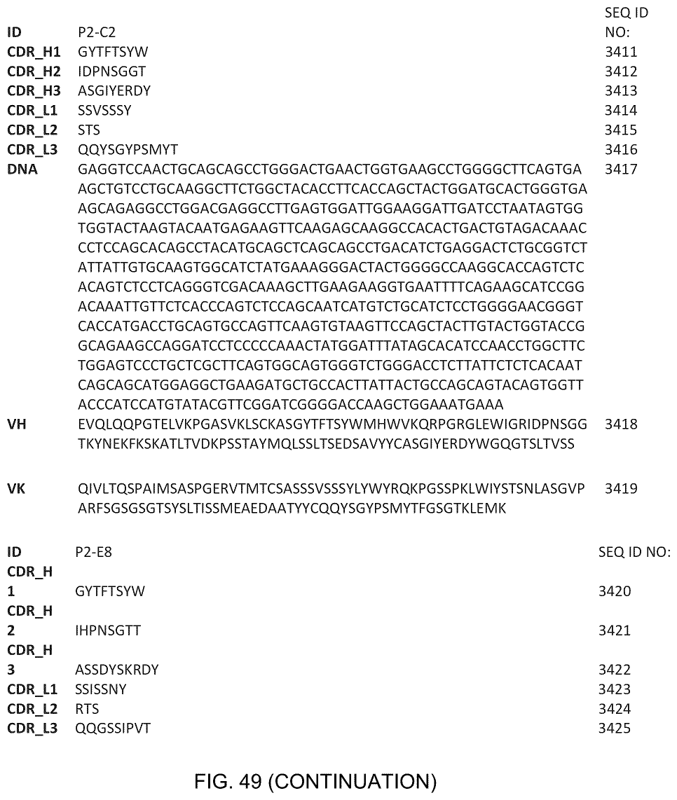

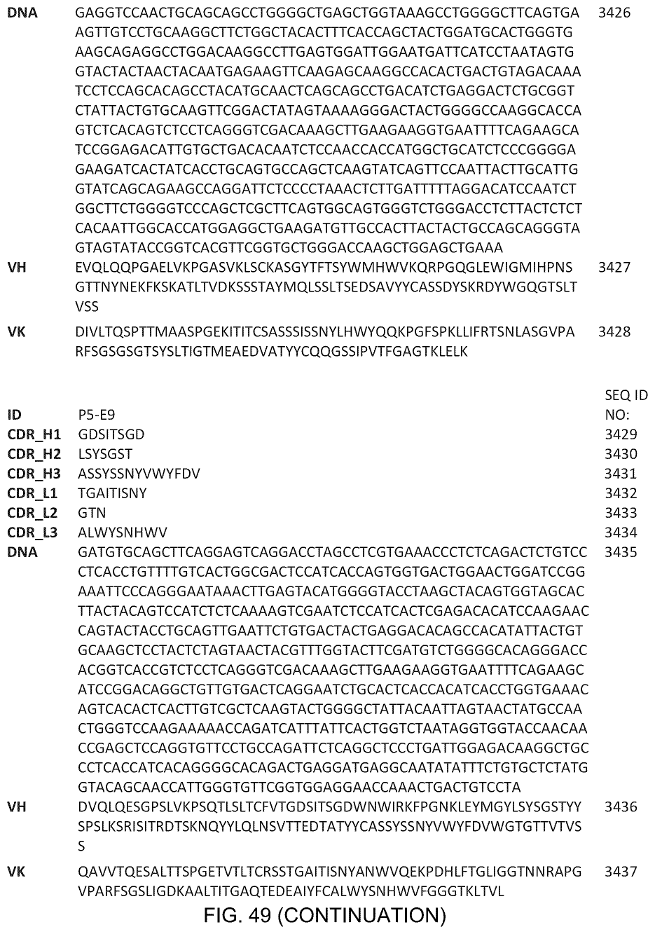

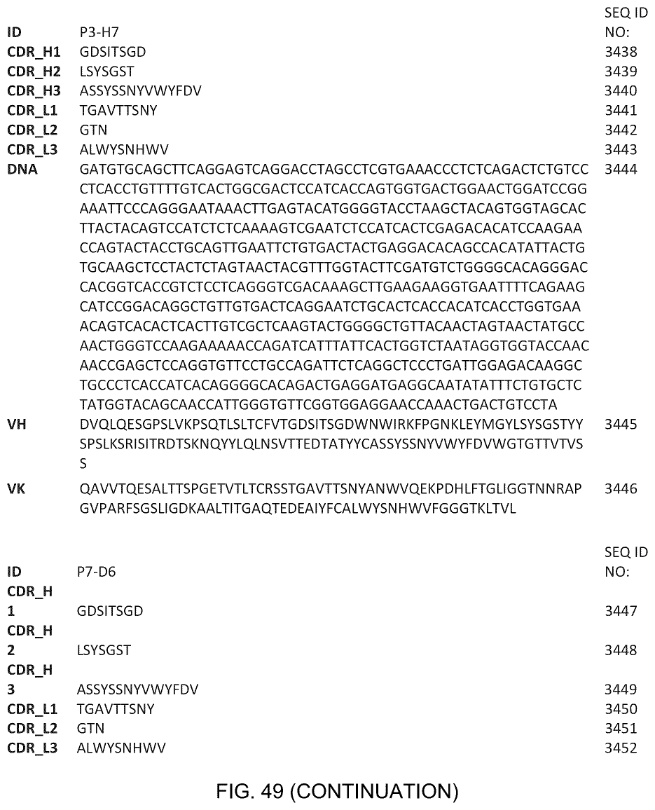

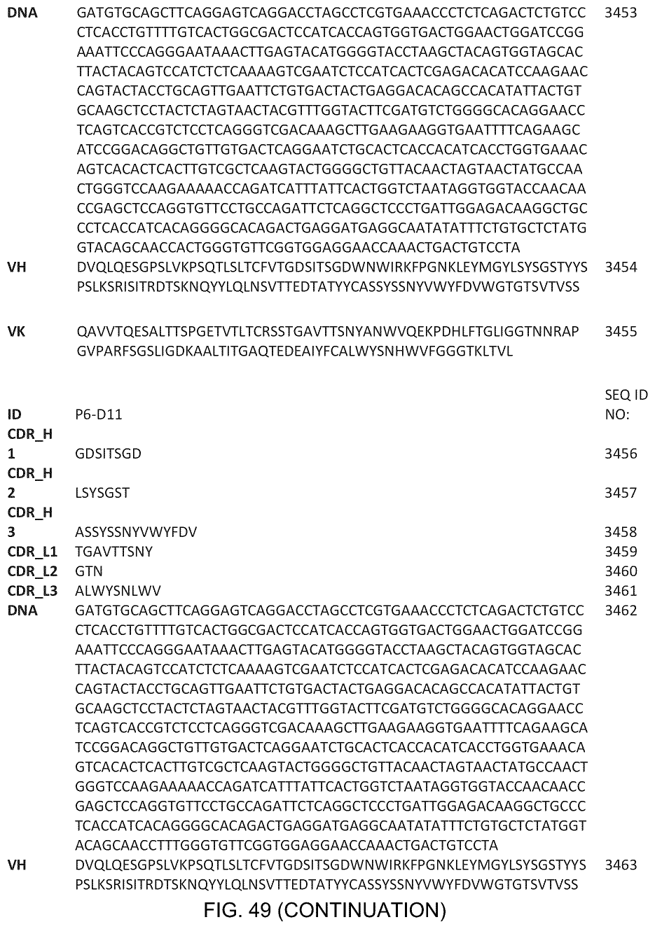

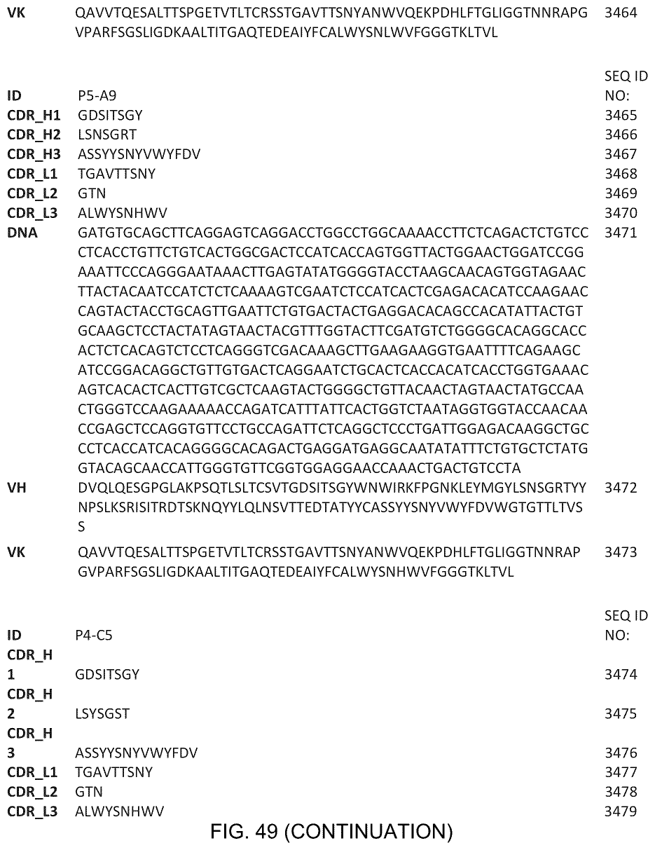

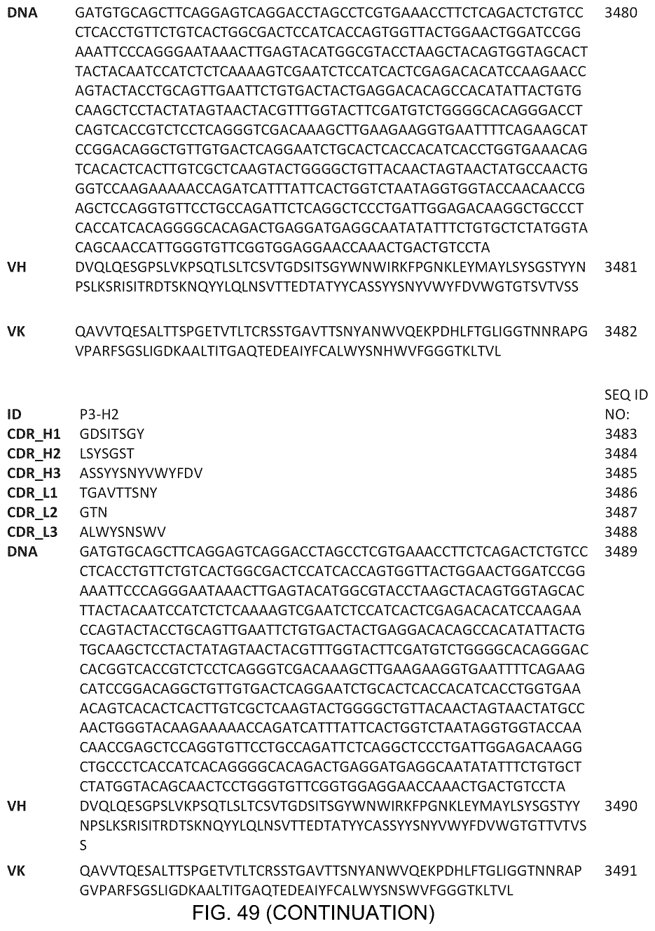

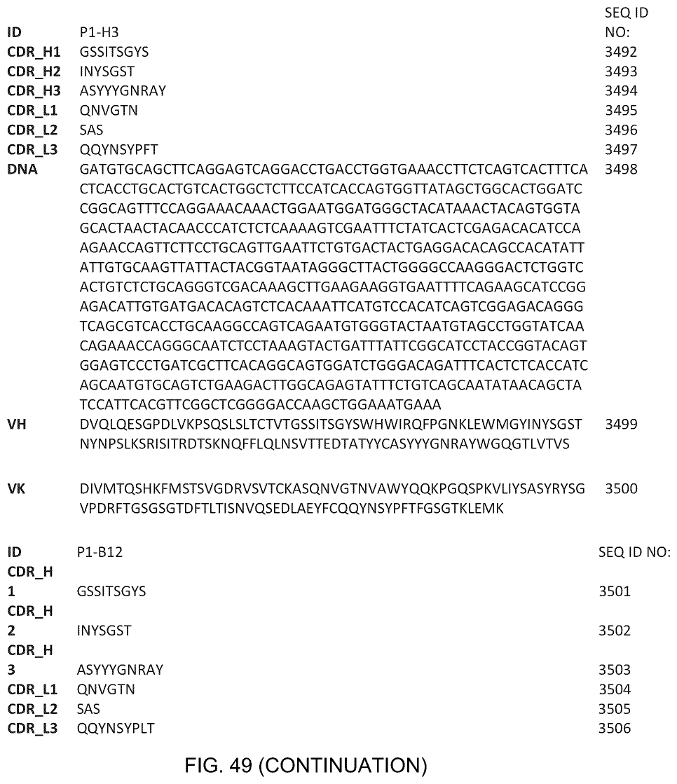

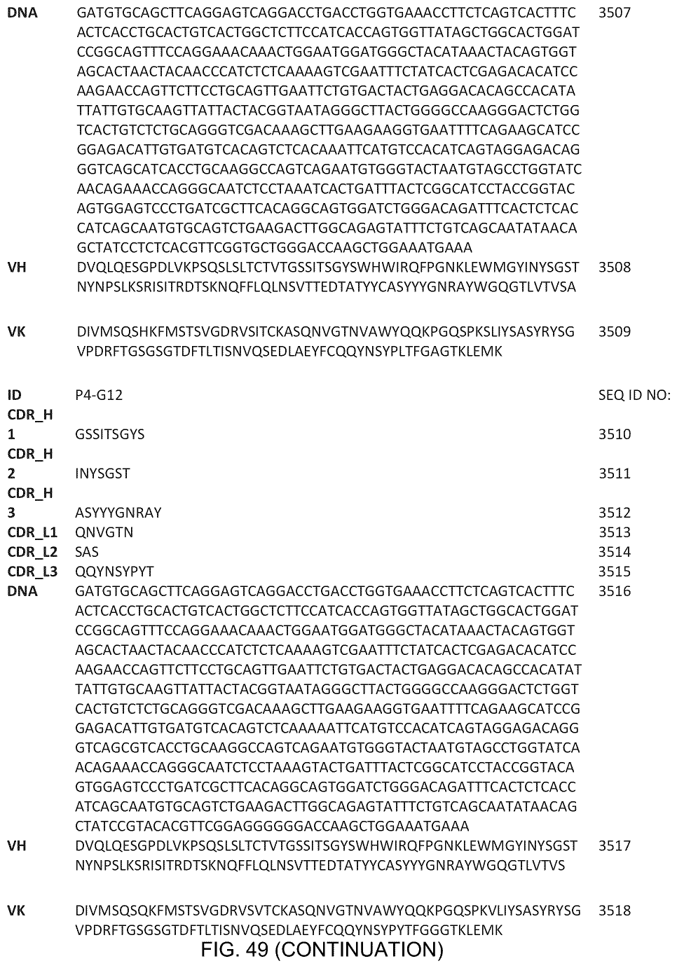

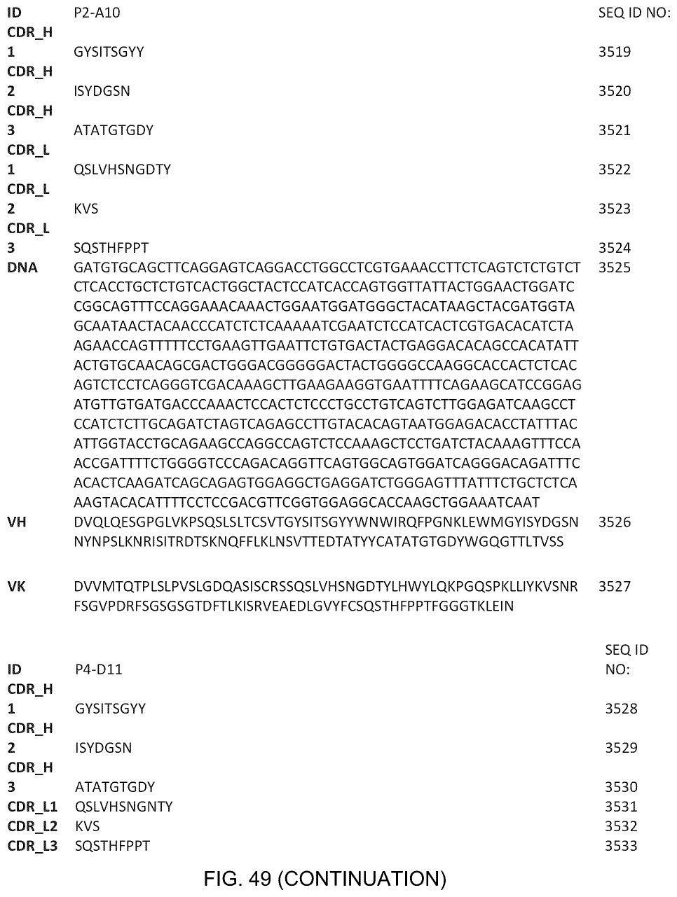

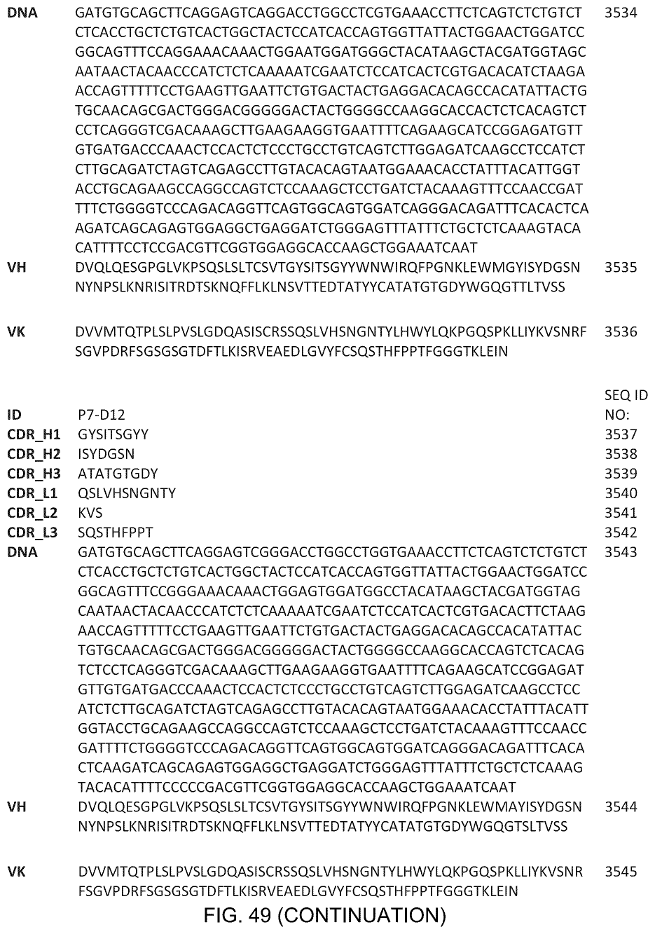

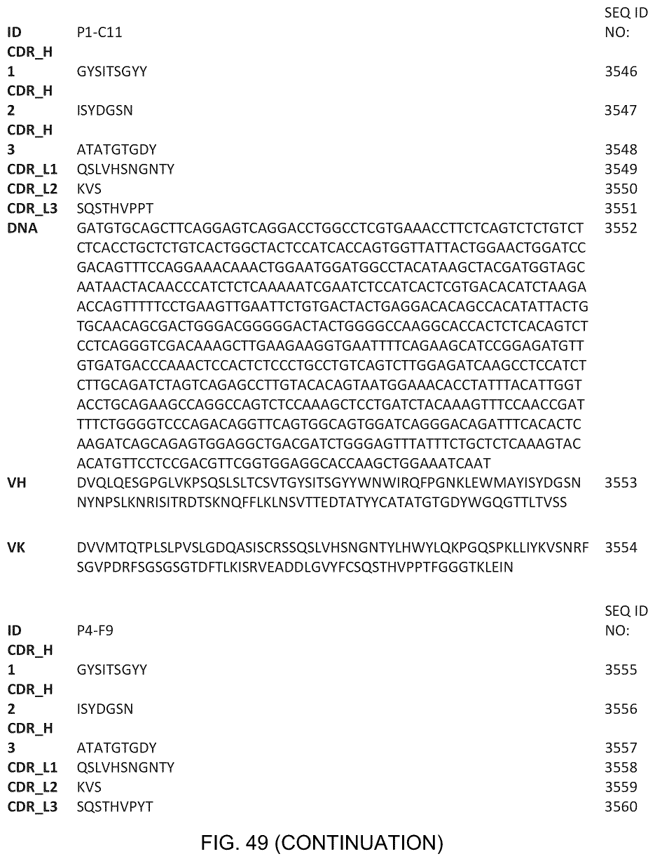

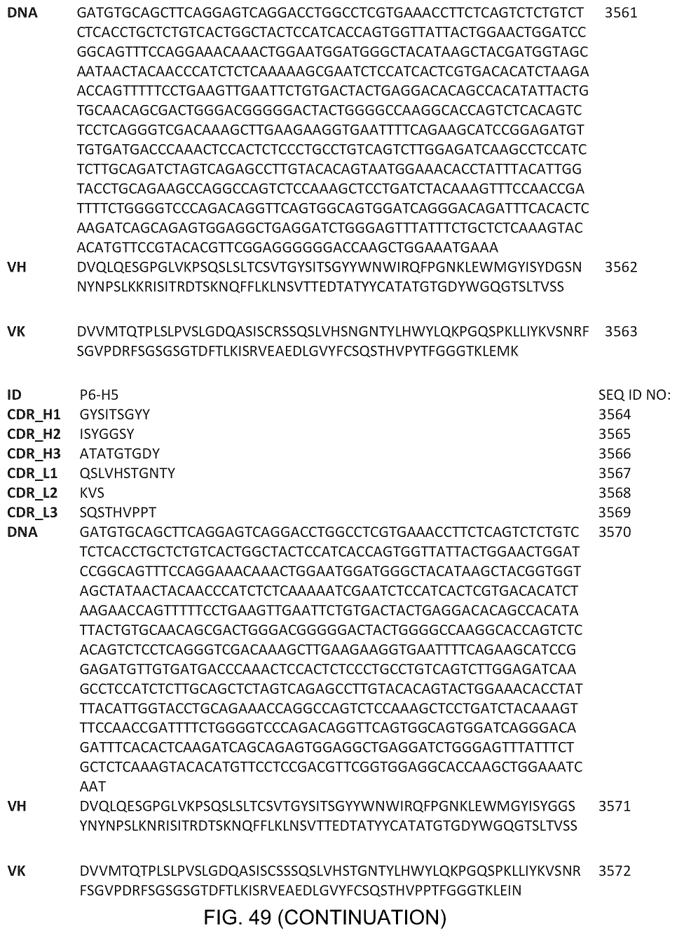

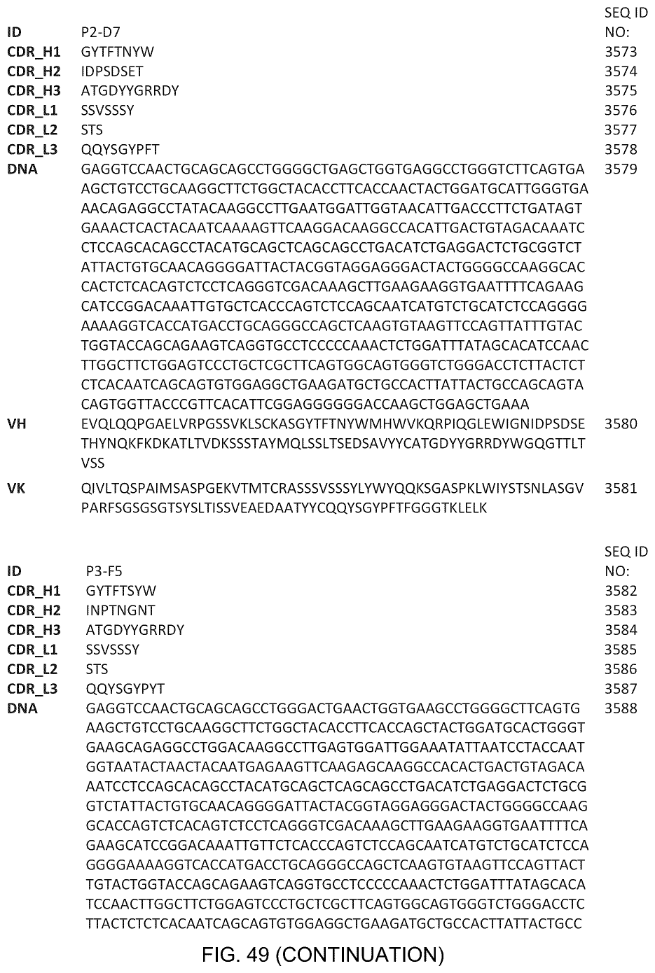

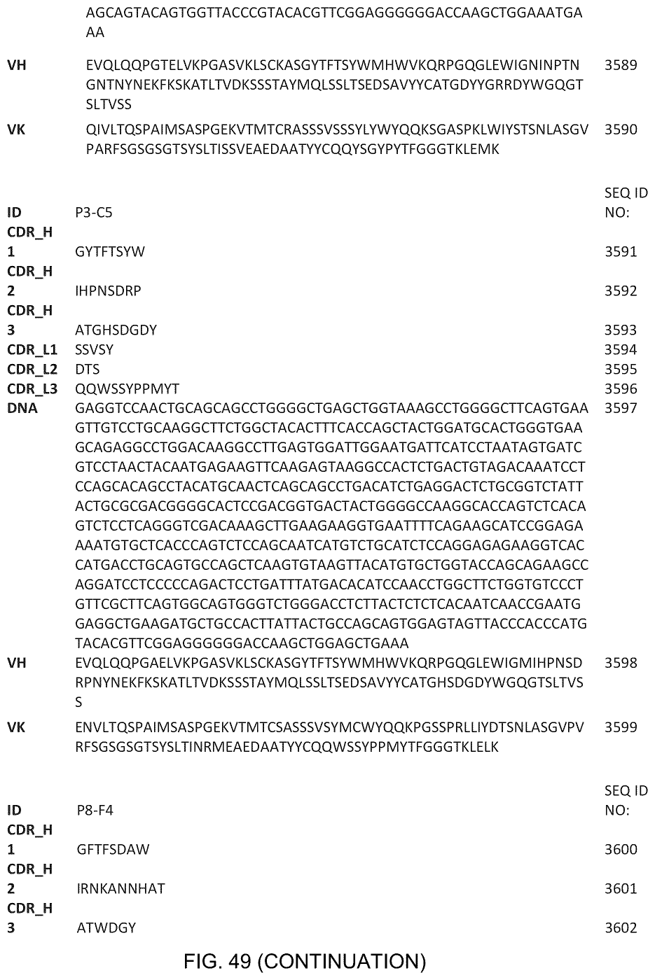

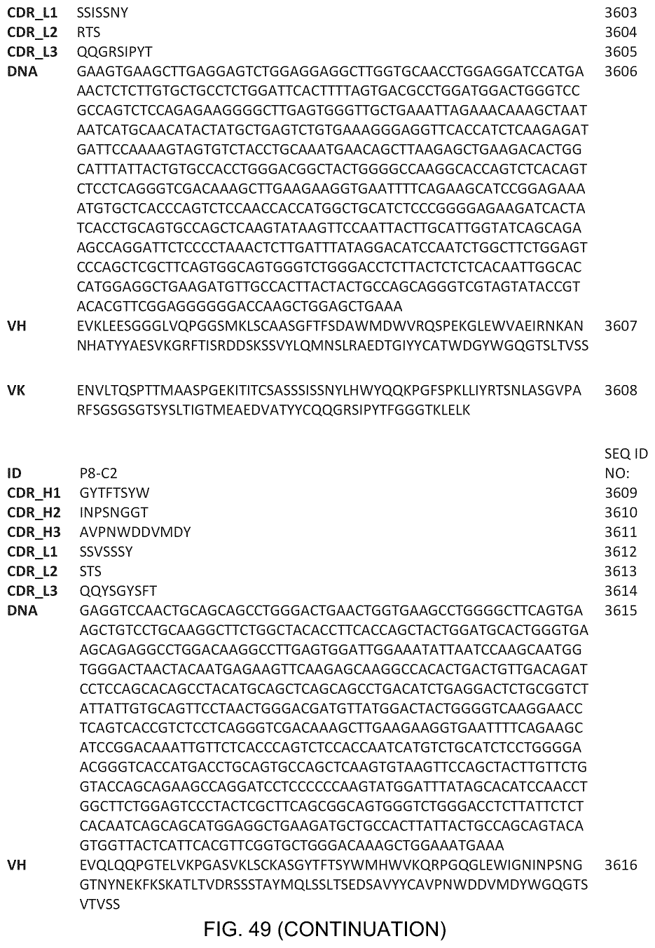

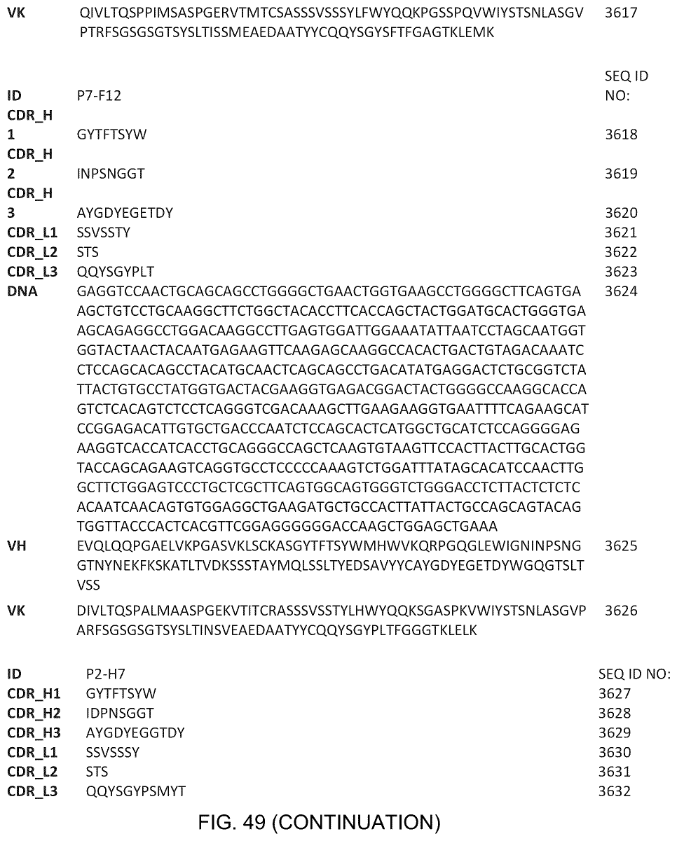

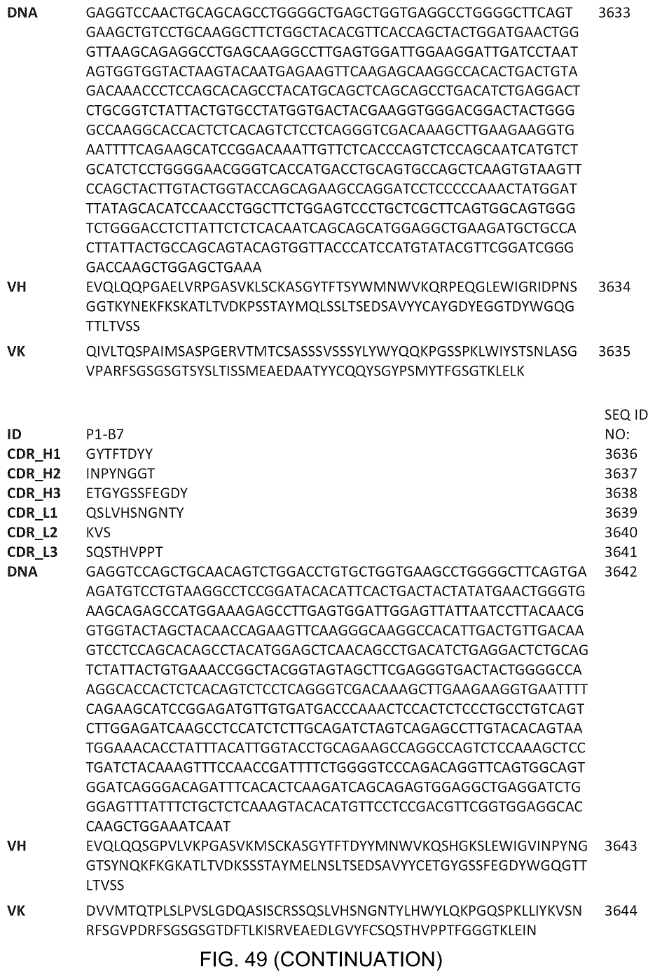

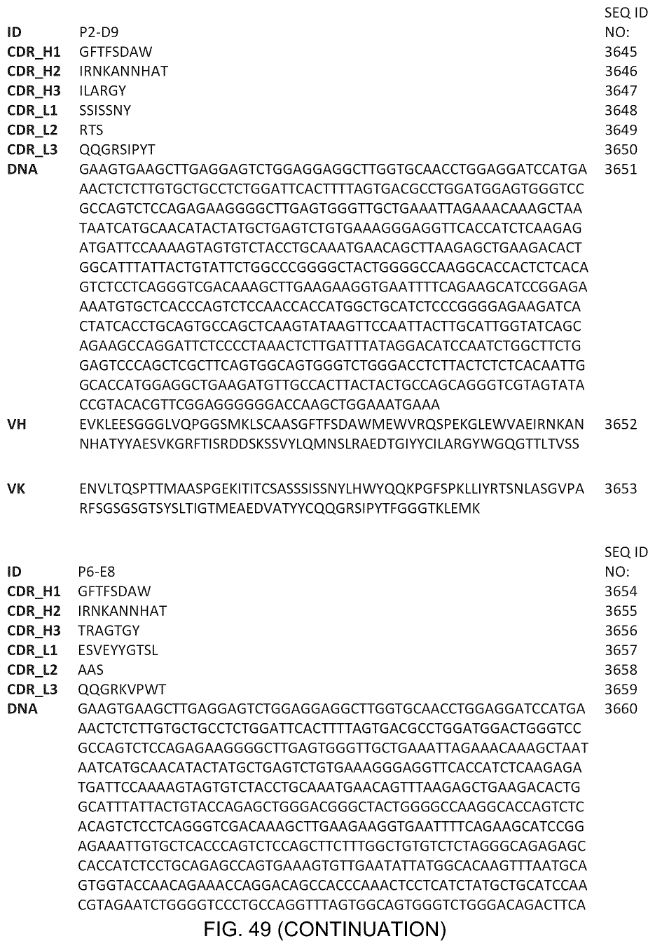

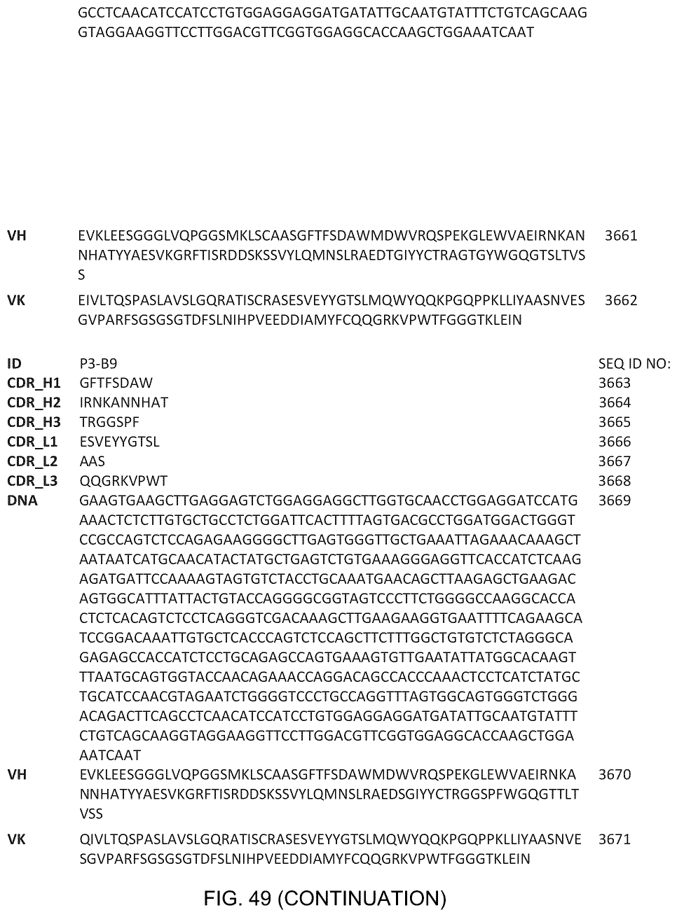

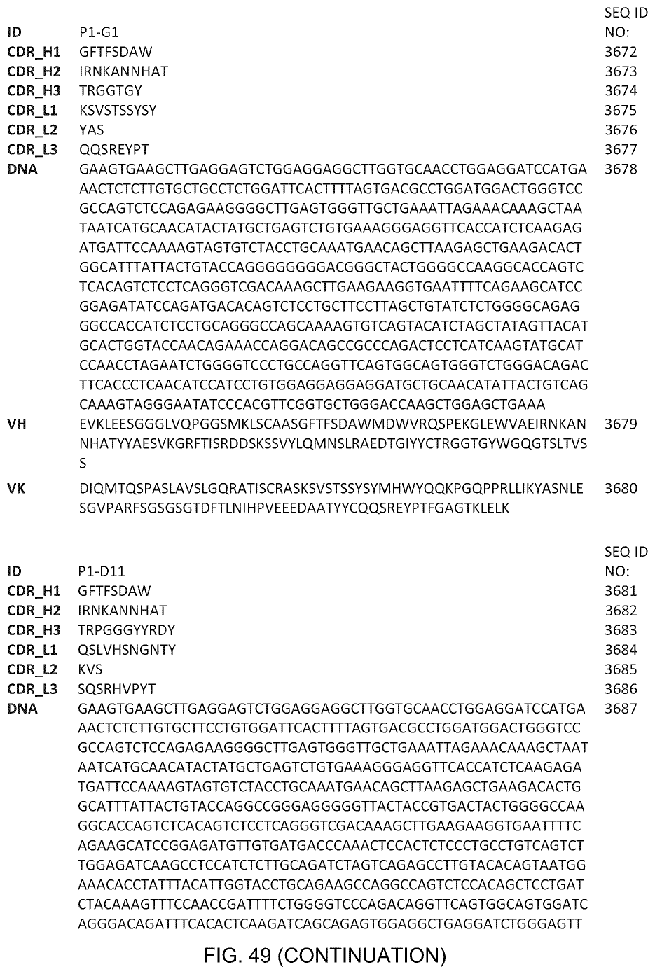

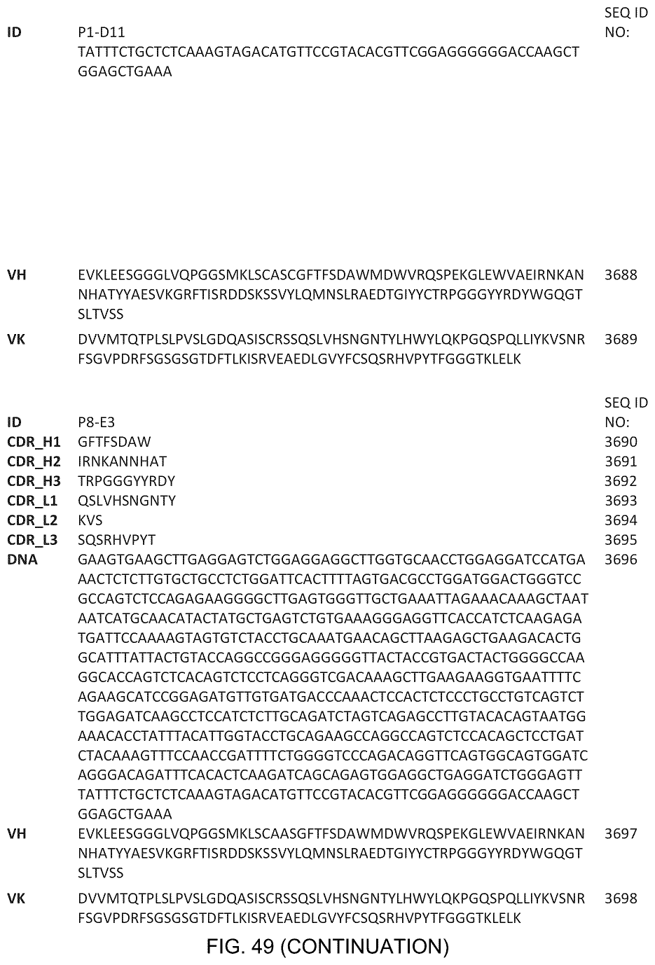

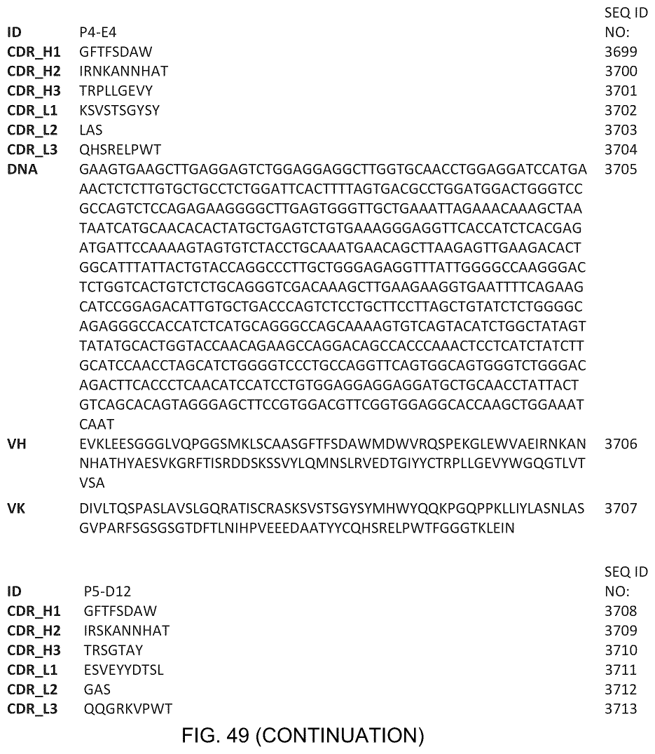

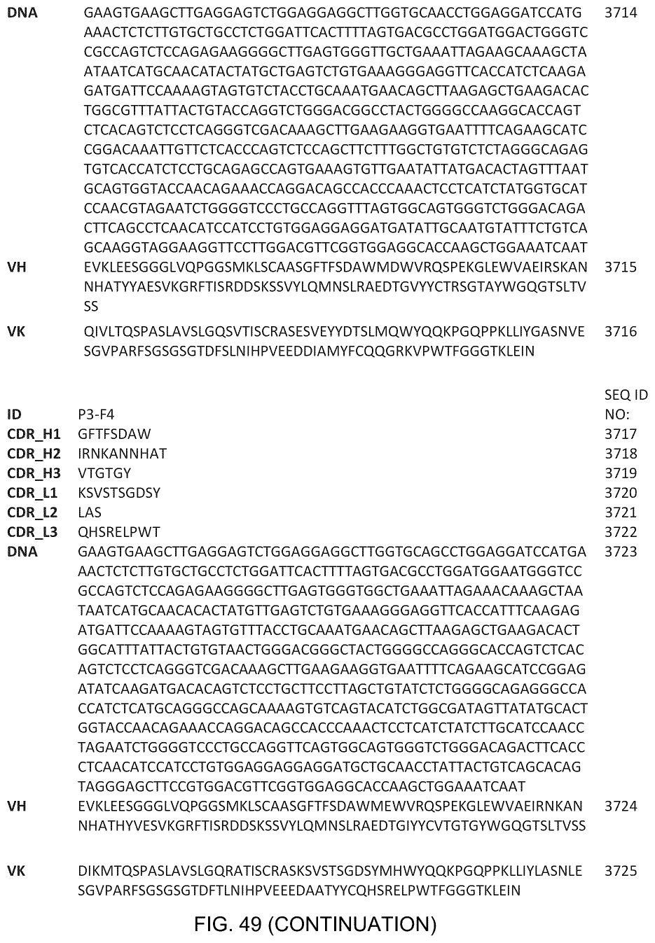

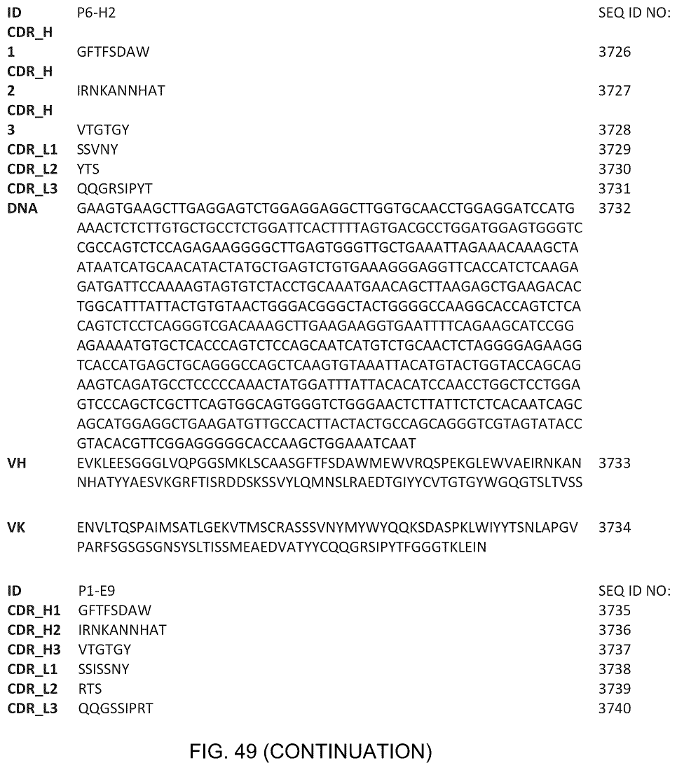

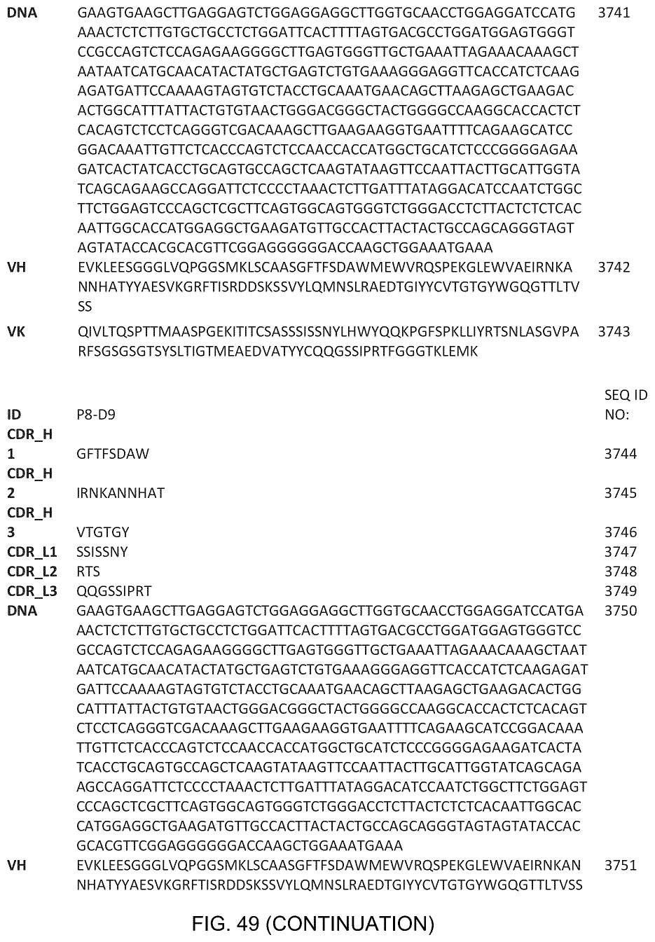

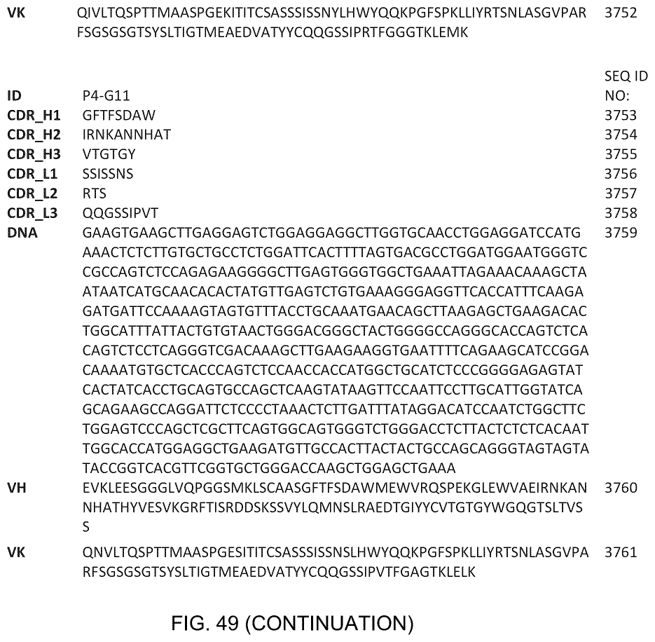

In another aspect, the present disclosure relates to a composition comprising an indinavir binding domain comprising: a) a variable heavy (VH) domain comprising a vhCDR1, vhCDR2, and vhCDR3 sequence; b) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, P5-H5, P2-D8, P3-E12, P6-C8, P7-G8, P6-D7, P6-D5, P3-A11, P5-C3, P3-H5, P7-H10, P7-C6, P2-C2, P2-E8, P5-E9, P3-H7, P7-D6, P6-D11, P5-A9, P4-C5, P3-H2, P1-H3, P1-B12, P4-G12, P2-A10, P4-D11, P7-D12, P1-C11, P4-F9, P6-H5, P2-D7, P3-F5, P3-C5, P8-F4, P8-C2, P7-F12, P2-H7, P1-B7, P2-D9, P6-E8, P3-B9, P1-G1, P1-D11, P8-E3, P4-E4, P5-D12, P3-F4, P6-H2, P1-E9, P8-D9, and P4-G11 (FIG. 49). In another aspect, the present disclosure relates to a composition according to claim A1 wherein said indinavir binding domain comprises a VH domain and VL domain selected from the group consisting of the VH and VL domains of P7-F7, P7-E7, P5-H5, P2-D8, P3-E12, P6-C8, P7-G8, P6-D7, P6-D5, P3-A11, P5-C3, P3-H5, P7-H10, P7-C6, P2-C2, P2-E8, P5-E9, P3-H7, P7-D6, P6-D11, P5-A9, P4-C5, P3-H2, P1-H3, P1-B12, P4-G12, P2-A10, P4-D11, P7-D12, P1-C11, P4-F9, P6-H5, P2-D7, P3-F5, P3-C5, P8-F4, P8-C2, P7-F12, P2-H7, P1-B7, P2-D9, P6-E8, P3-B9, P1-G1, P1-D11, P8-E3, P4-E4, P5-D12, P3-F4, P6-H2, P1-E9, P8-D9, and P4-G11 (FIG. 49). In some embodiments, said composition is a fusion protein. In some embodiments, the composition comprises an indinavir binding domain comprising scFv means for binding indinavir. In some embodiments, the composition comprises an indinavir binding domain comprising Fab means for binding indinavir. In some embodiments, the present disclosure relates to a nucleic acid composition comprising a polynucleotide encoding said composition herein. In some embodiments, the present disclosure relates to an expression vector composition comprising the nucleic acid composition herein. In some embodiments, the present disclosure relates to a host cell comprising the expression vector composition herein. In some embodiments, the present disclosure relates to a method of making the composition herein, the method comprising: an immunization campaign, validation of murine indinavir binders, humanization of the murine indinavir binders, generation of the composition herein, and validation of the composition herein.

b. Indinavir-Complex Binding Domains

As discussed herein, one of the iCID pair is an indinavir-complex binding domain, which are generally ABDs that bind the complex of the first iCID when complexed with indinavir. In some embodiments, the indinavir-complex binding domain comprises an indinavir-complex binding domain comprising: a) a variable heavy (VH) domain comprising a vhCDR1, vhCDR2, and vhCDR3 sequence; b) a variable light (VL) domain comprising a vlCDR1, vlCDR2, and vlCDR3 sequence. In some embodiments, the iCID is an scFv comprises an indinavir-complex binding domain comprising: a) a variable heavy (VH) domain comprising a vhCDR1, vhCDR2, and vhCDR3 sequence; b) a variable light (VL) domain comprising a vlCDR1, vlCDR2, and vlCDR3 sequence. In one aspect, the composition of the present disclosure comprises of an indinavir-complex binding domain comprising: a) a variable heavy (VH) domain comprising a vhCDR1, vhCDR2, and vhCDR3 sequence; b) a variable light (VL) domain comprising a vlCDR1, vlCDR2, and vlCDR3 sequence.

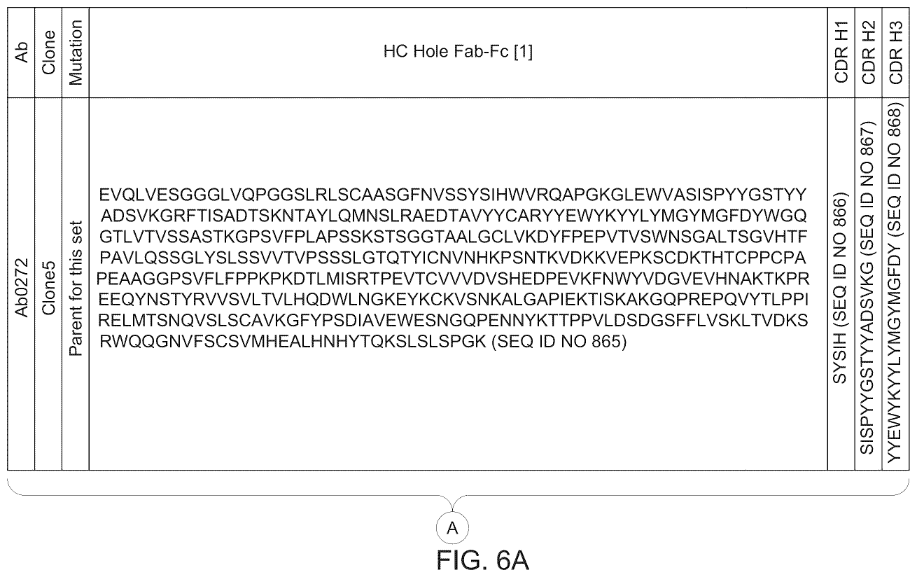

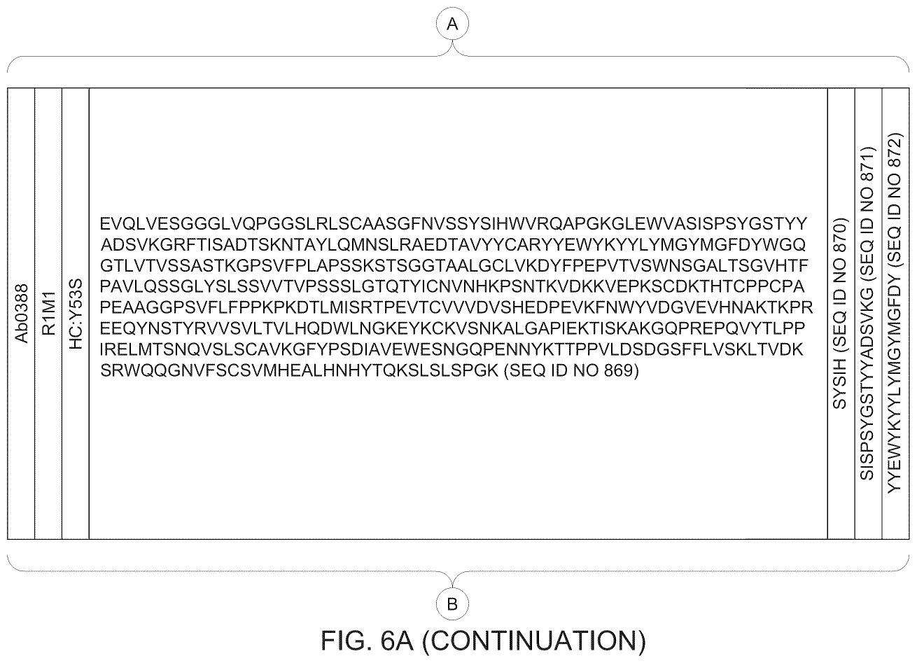

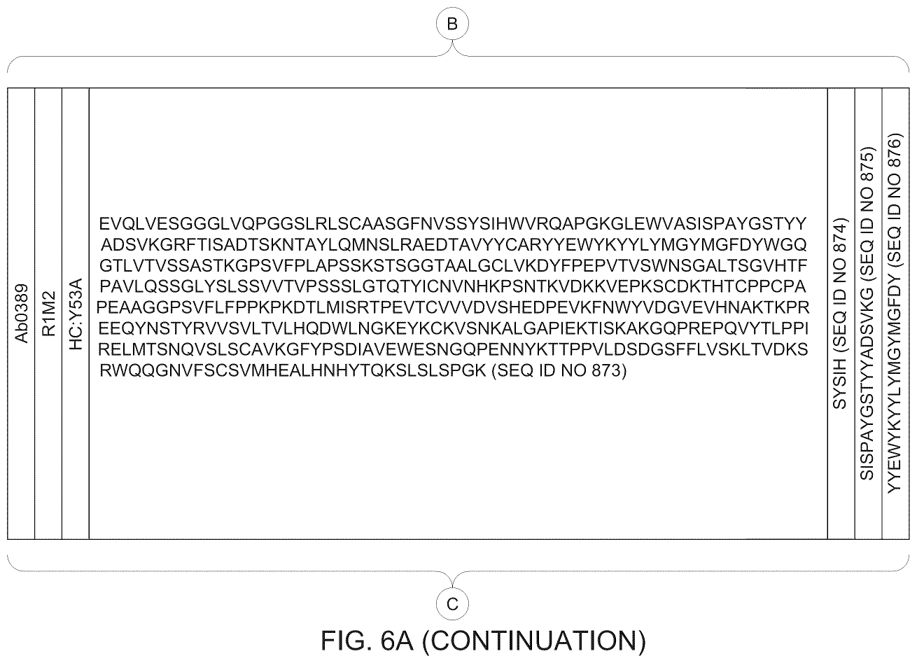

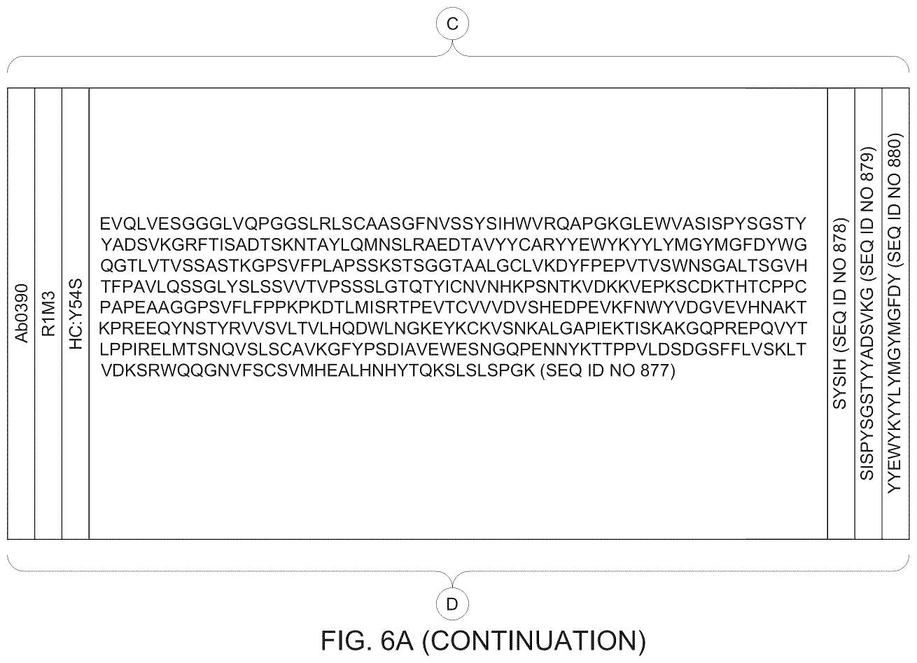

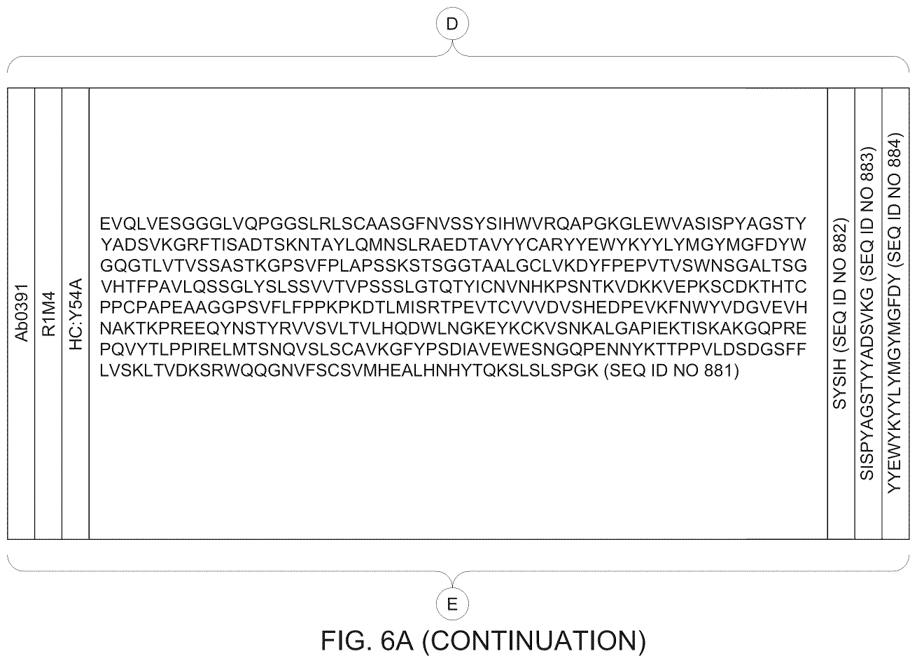

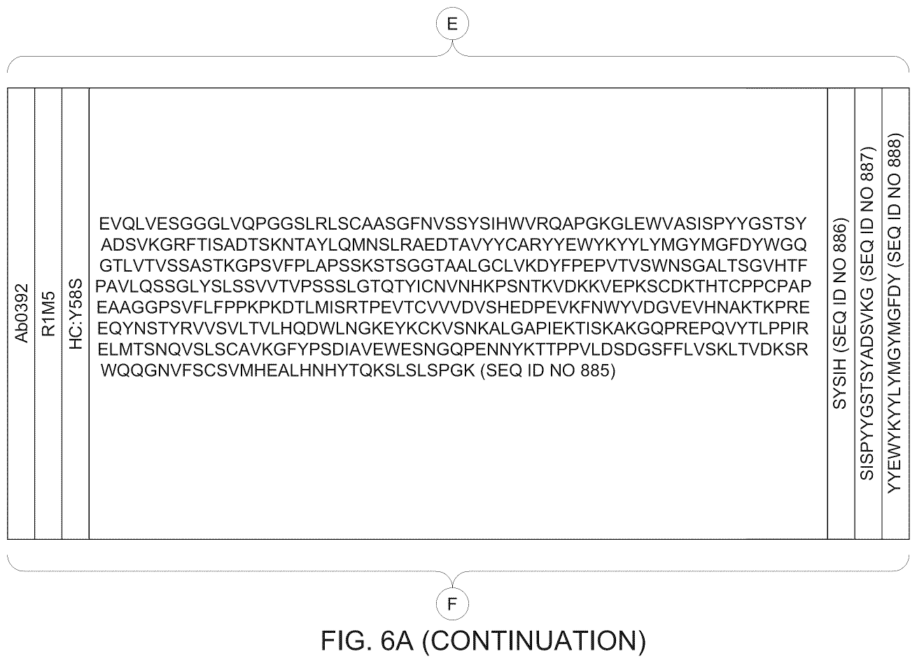

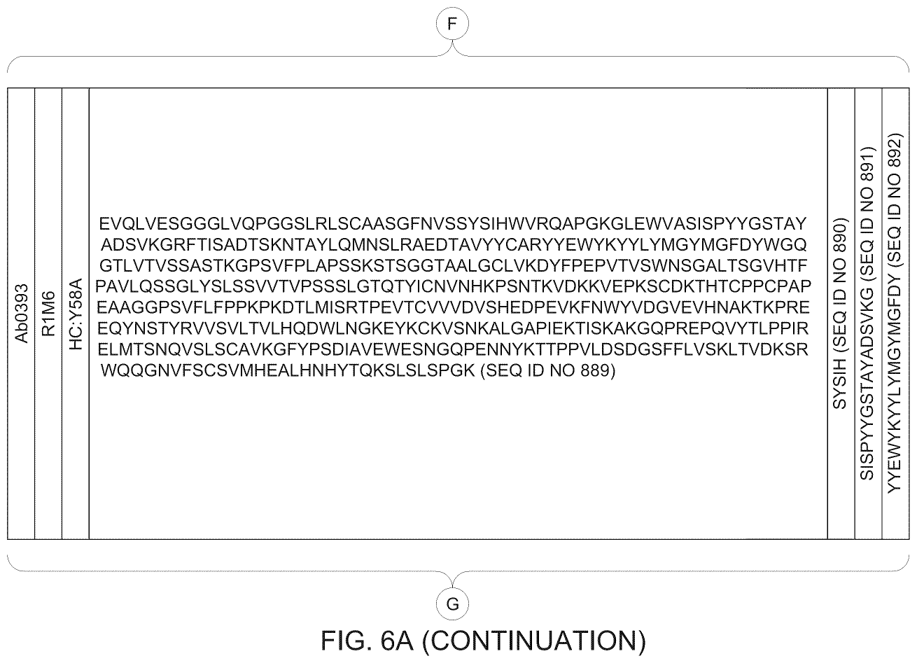

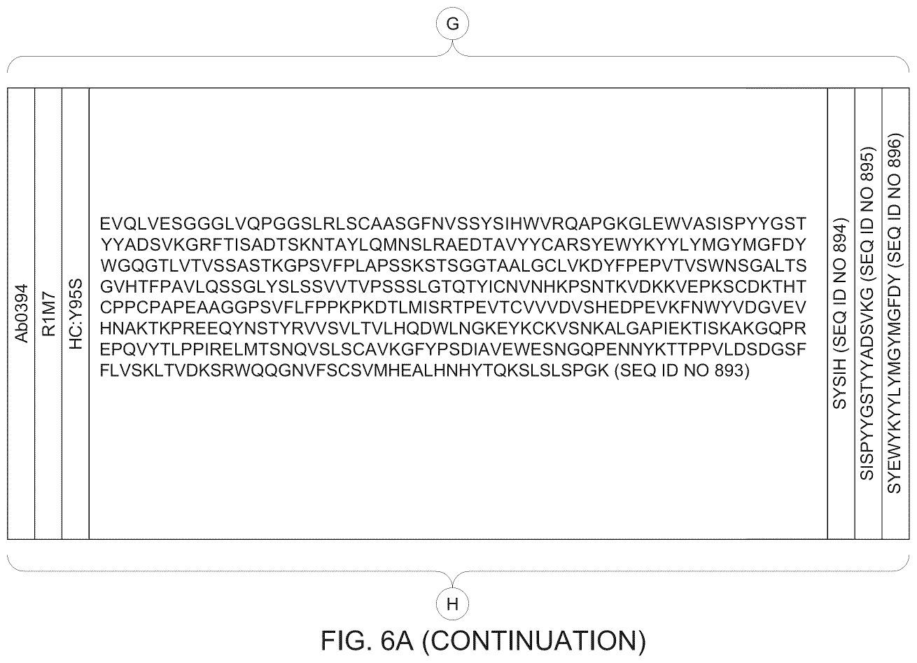

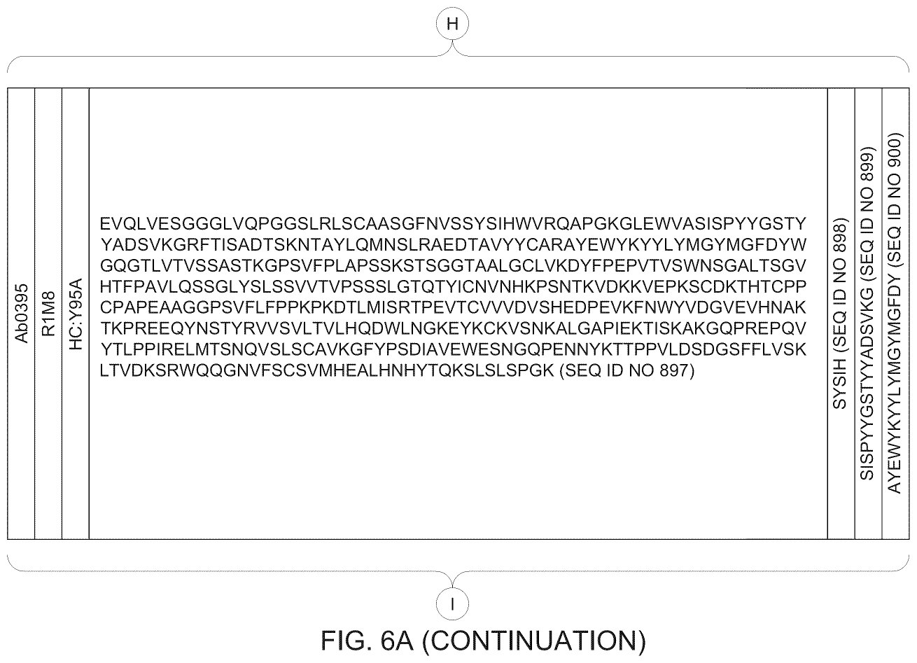

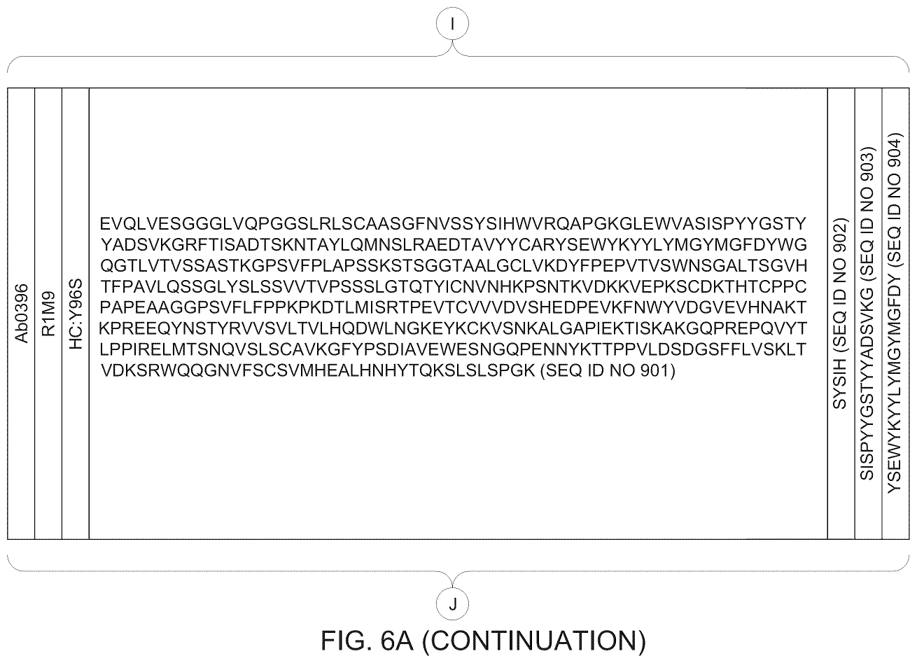

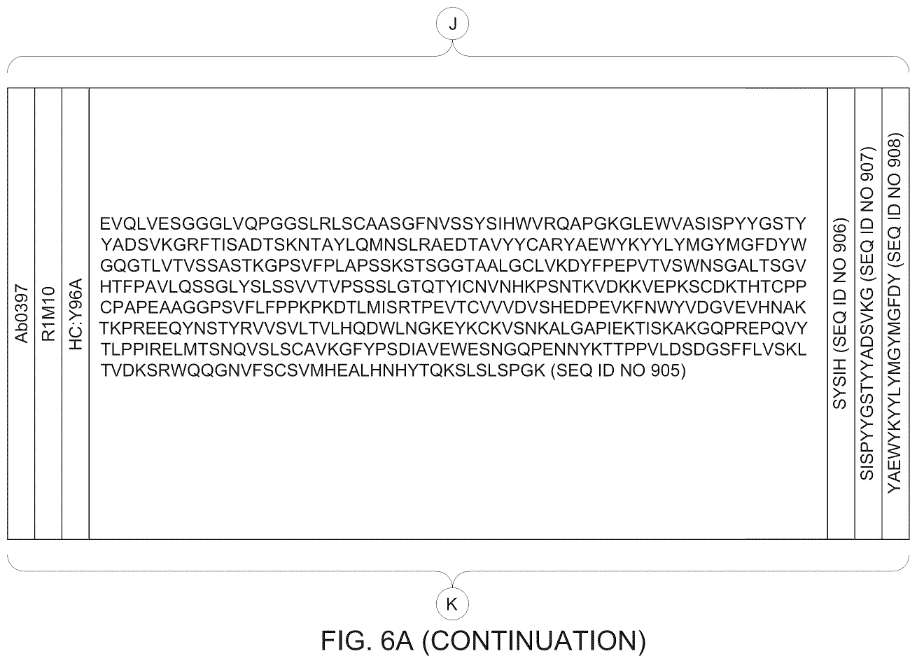

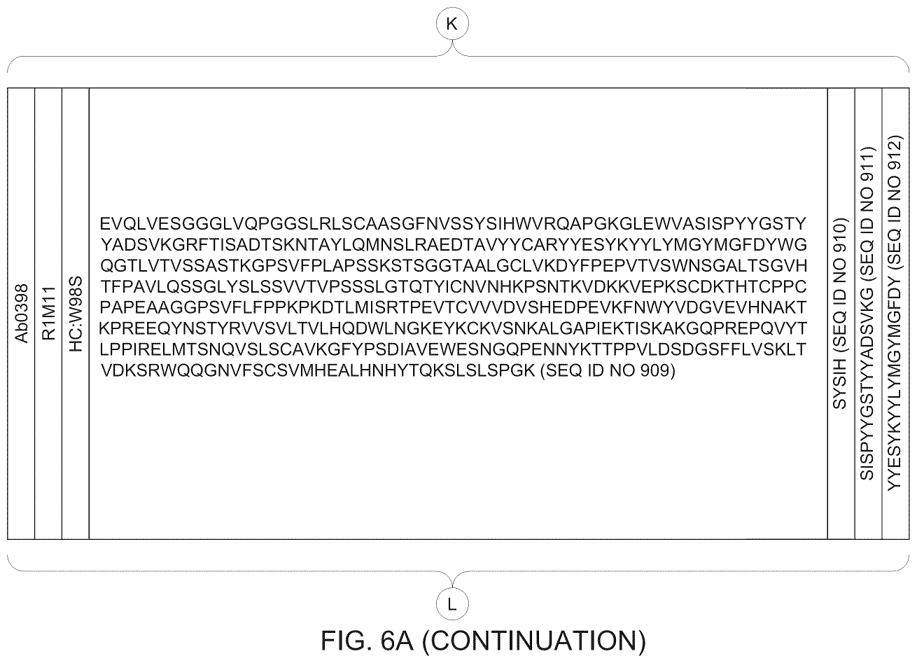

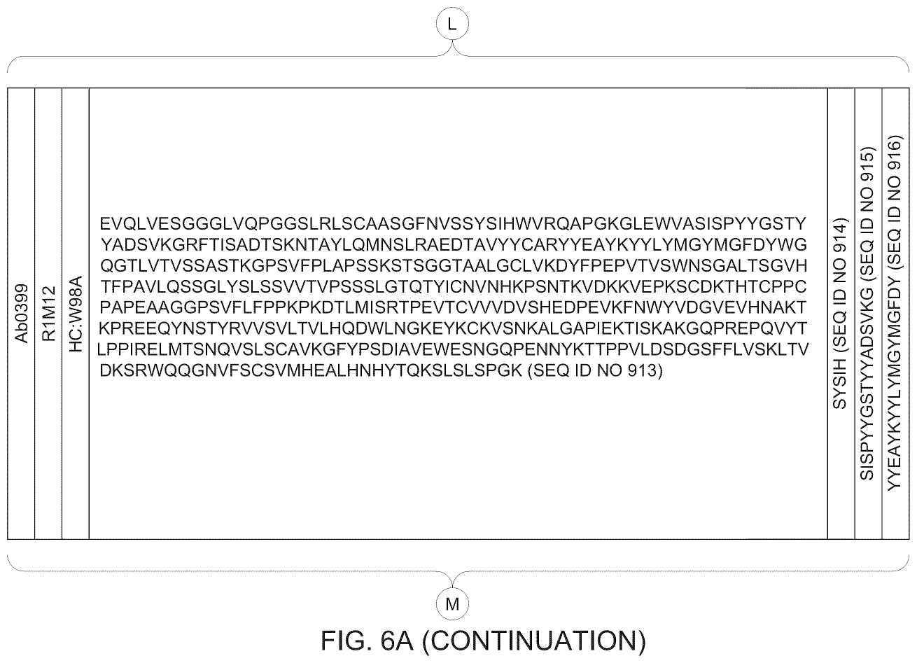

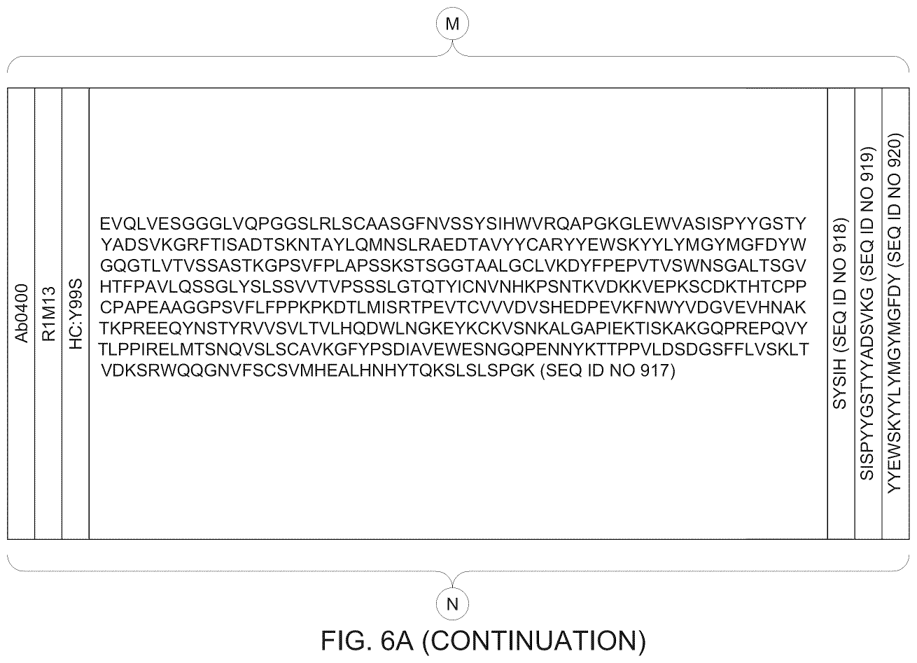

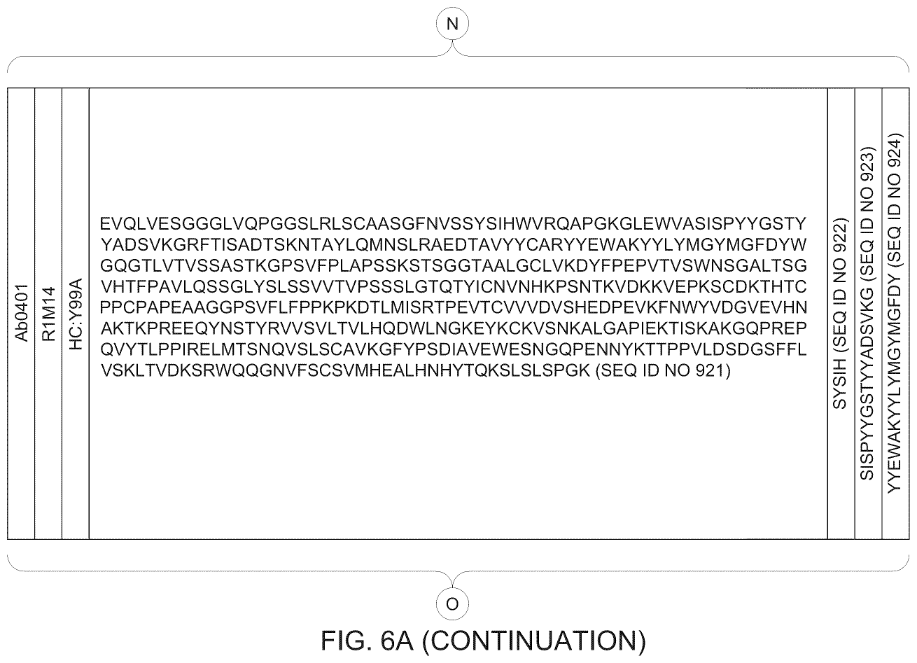

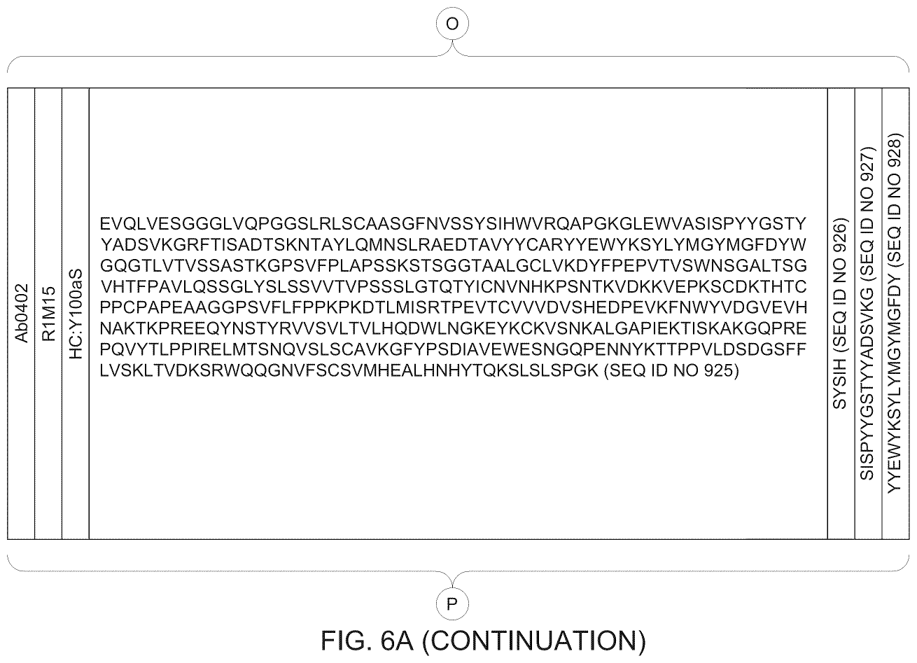

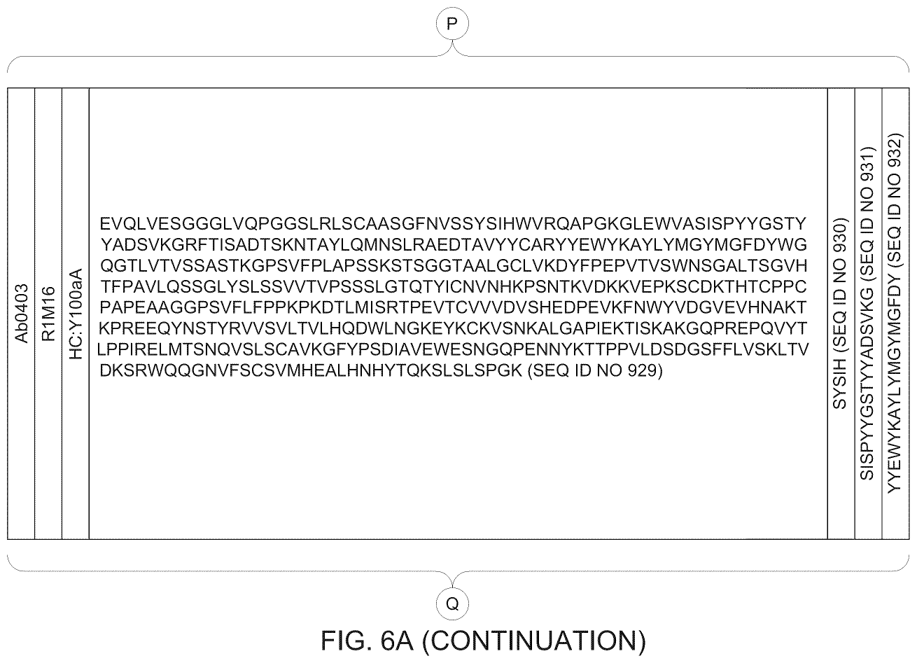

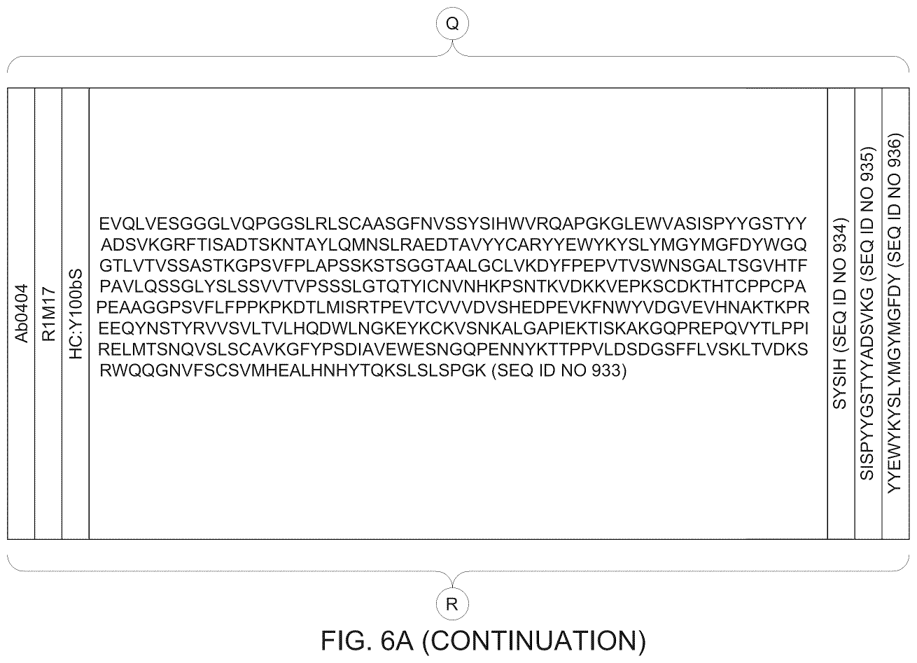

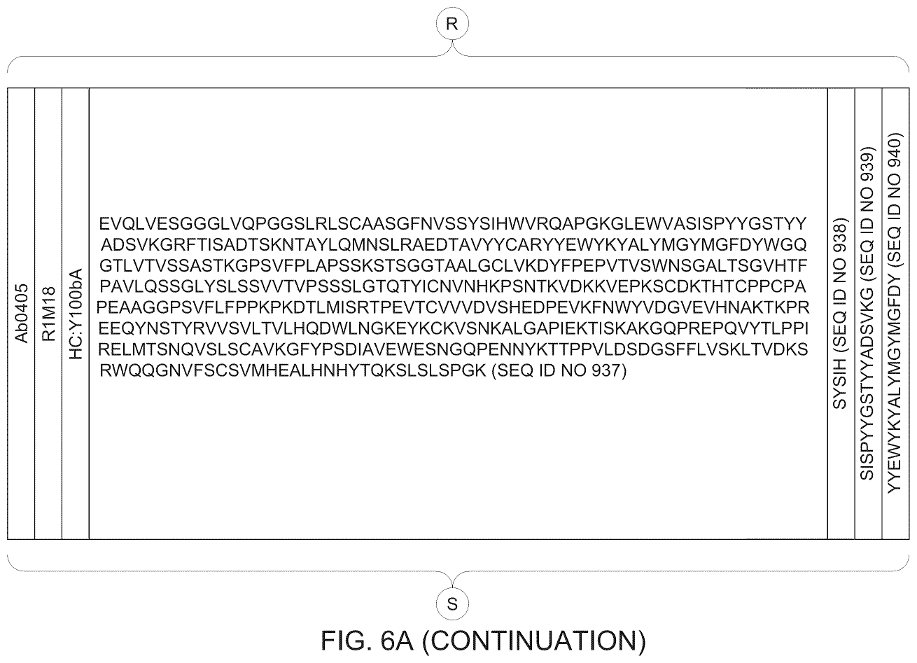

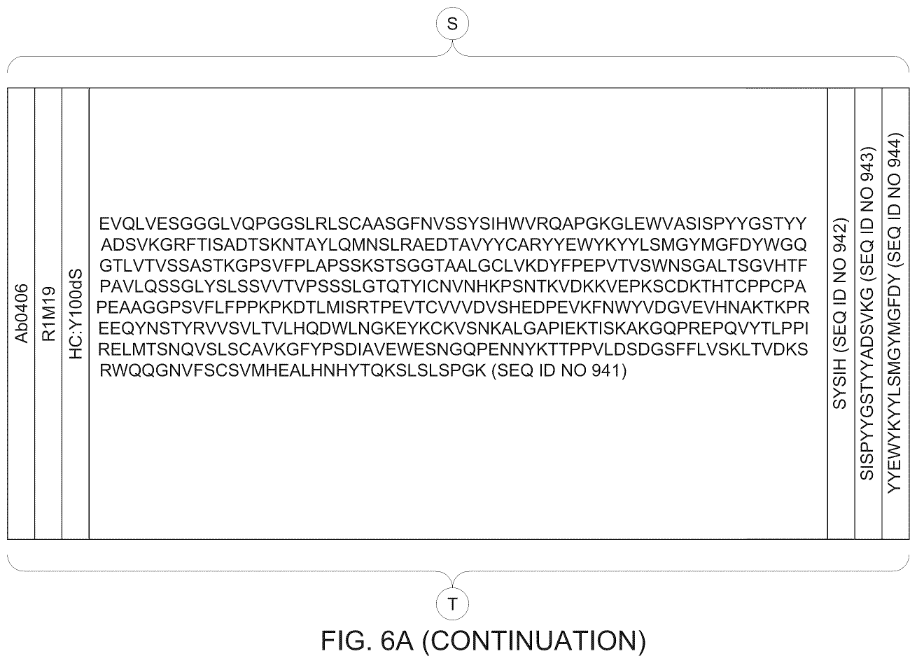

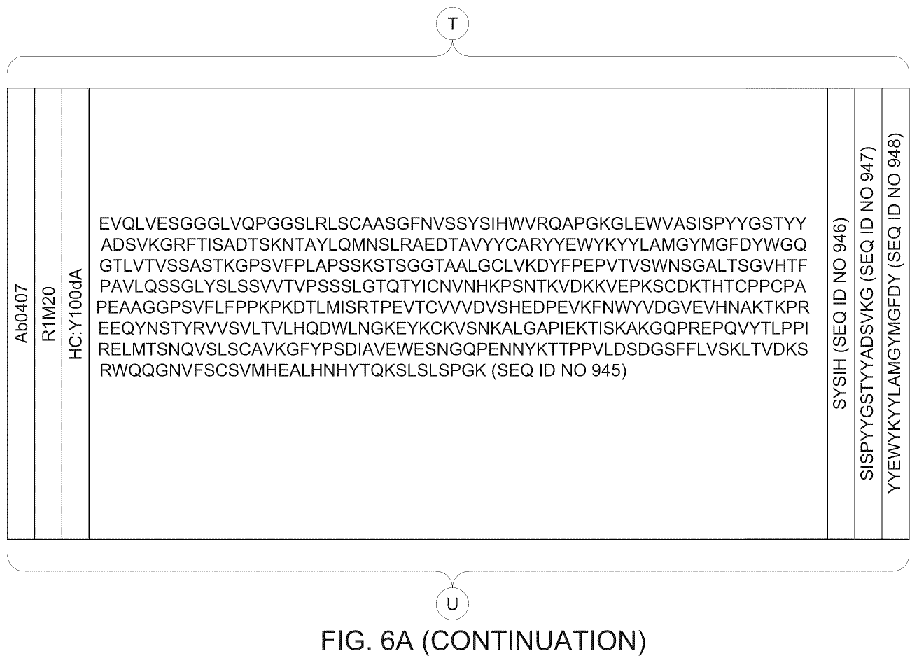

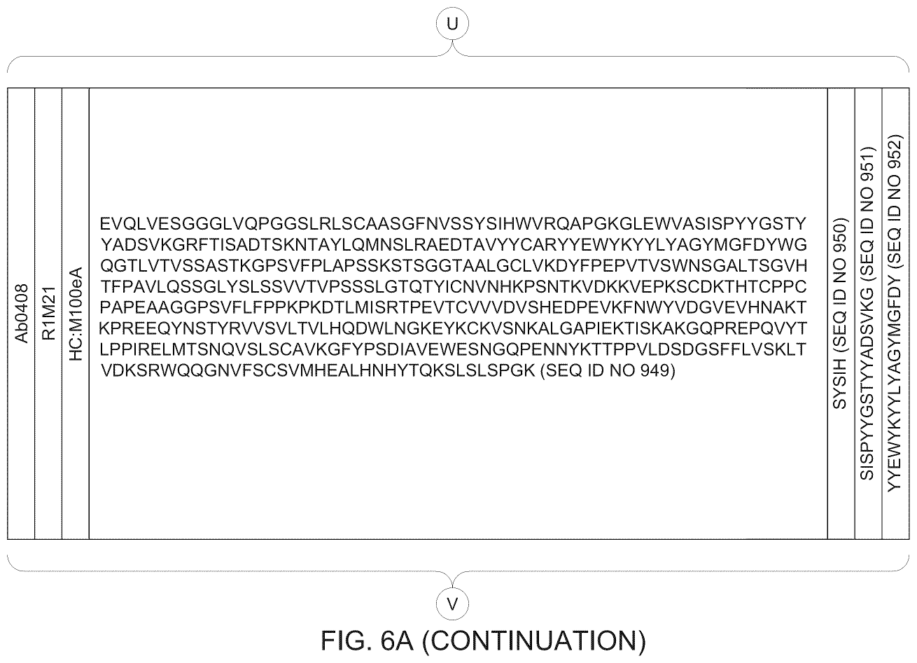

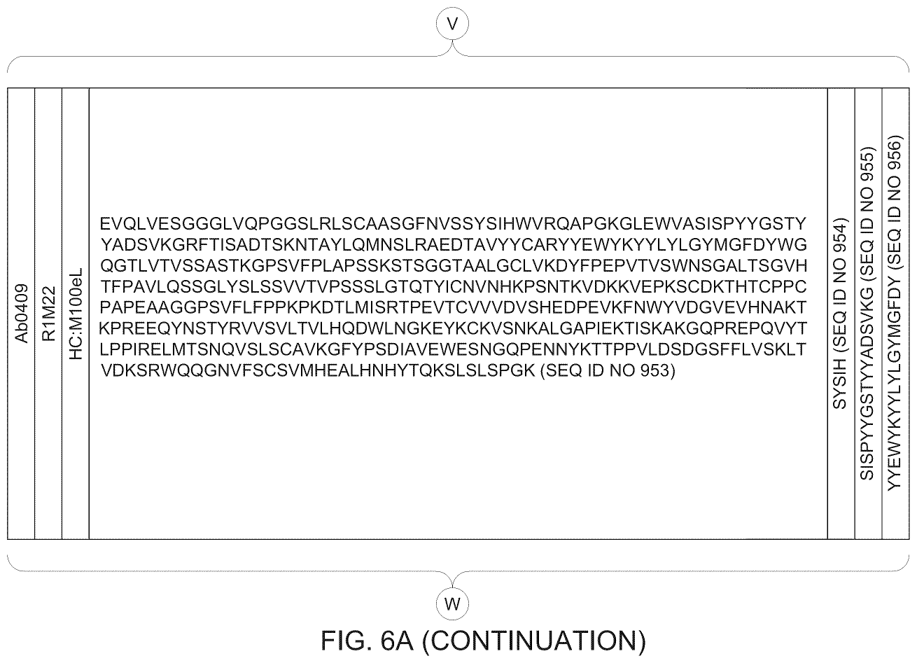

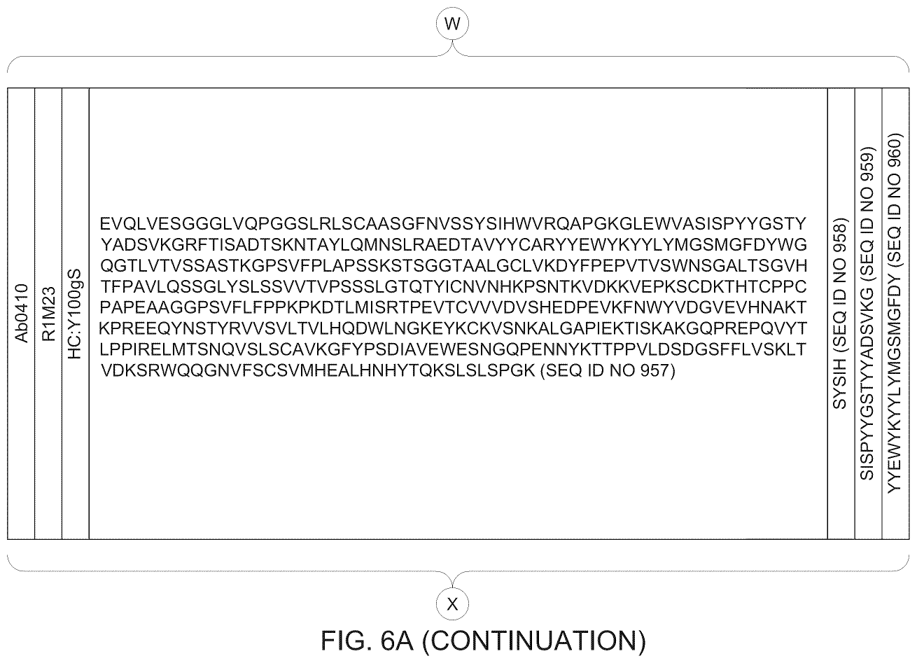

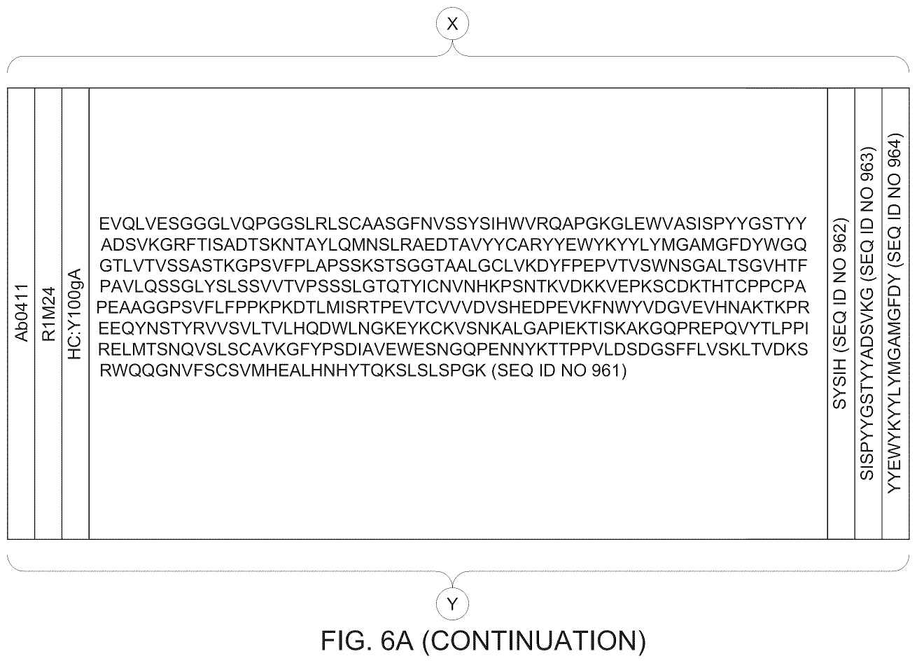

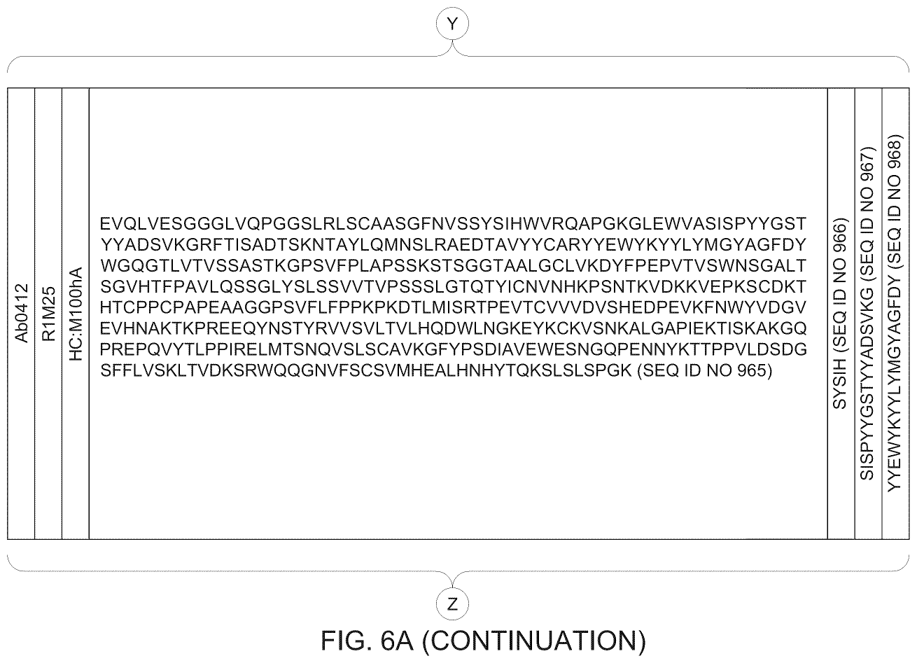

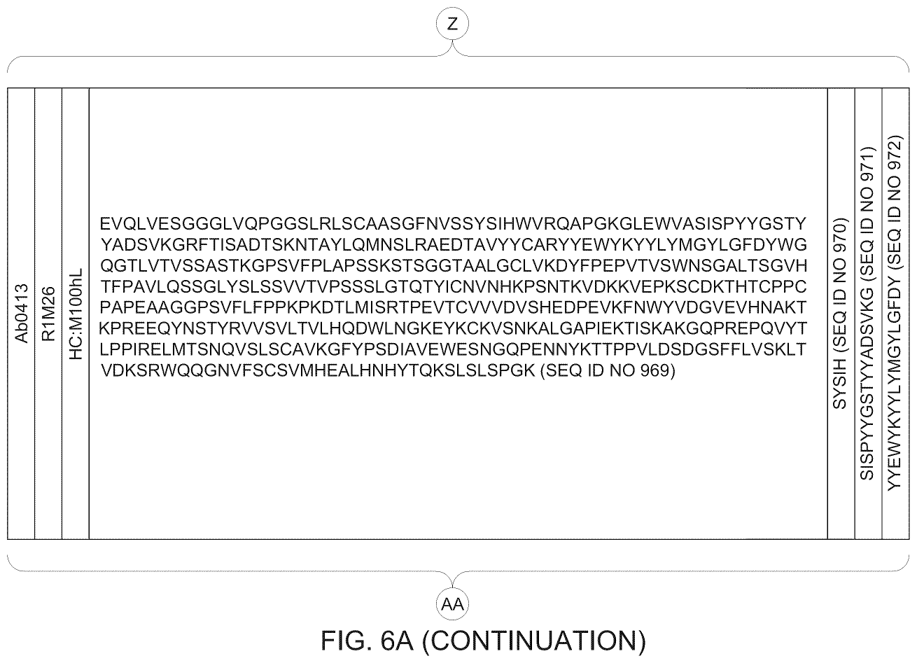

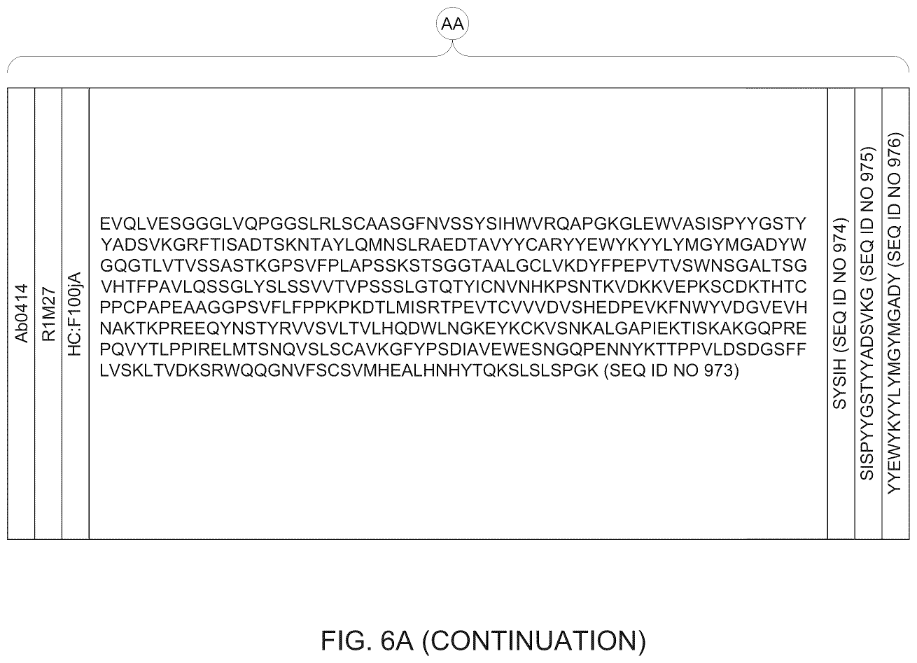

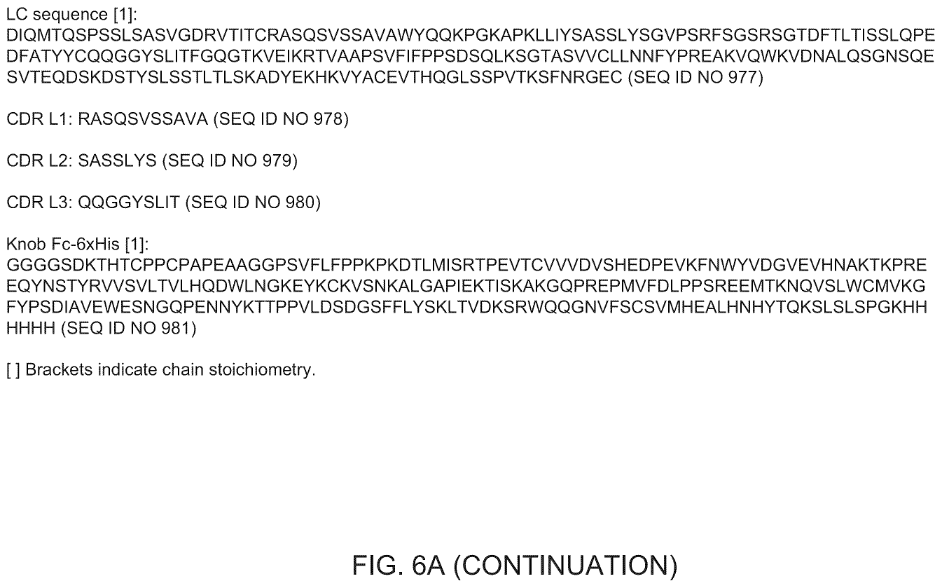

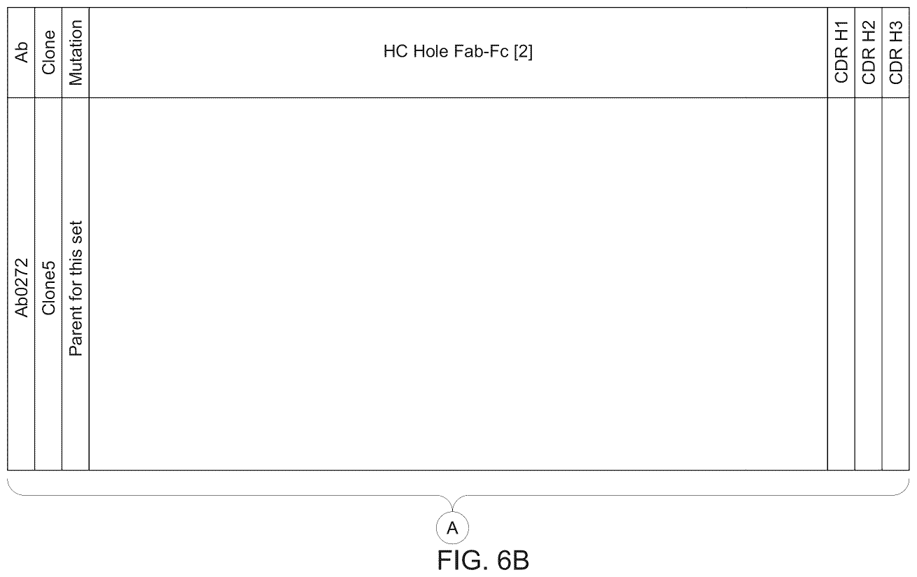

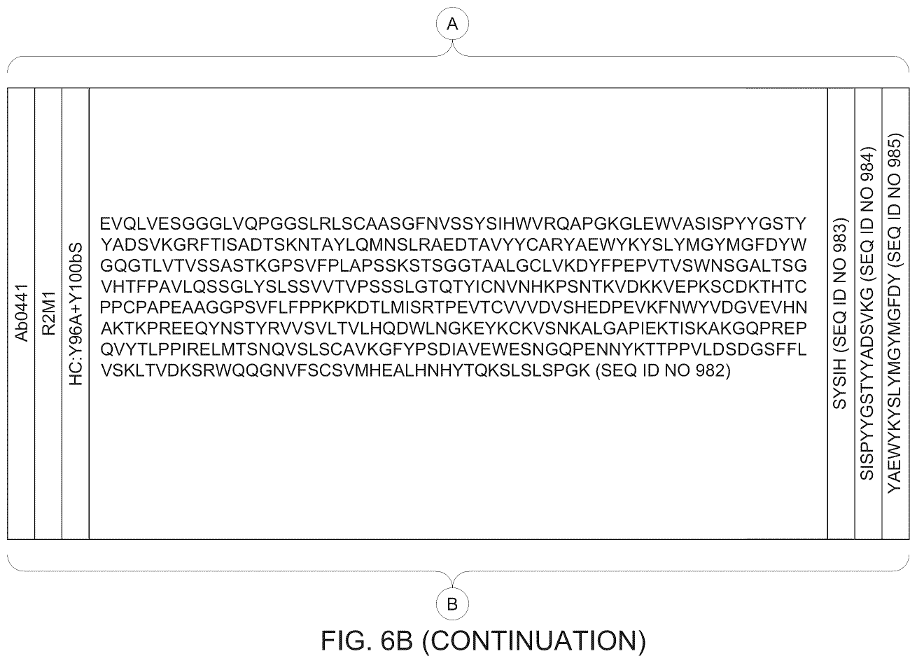

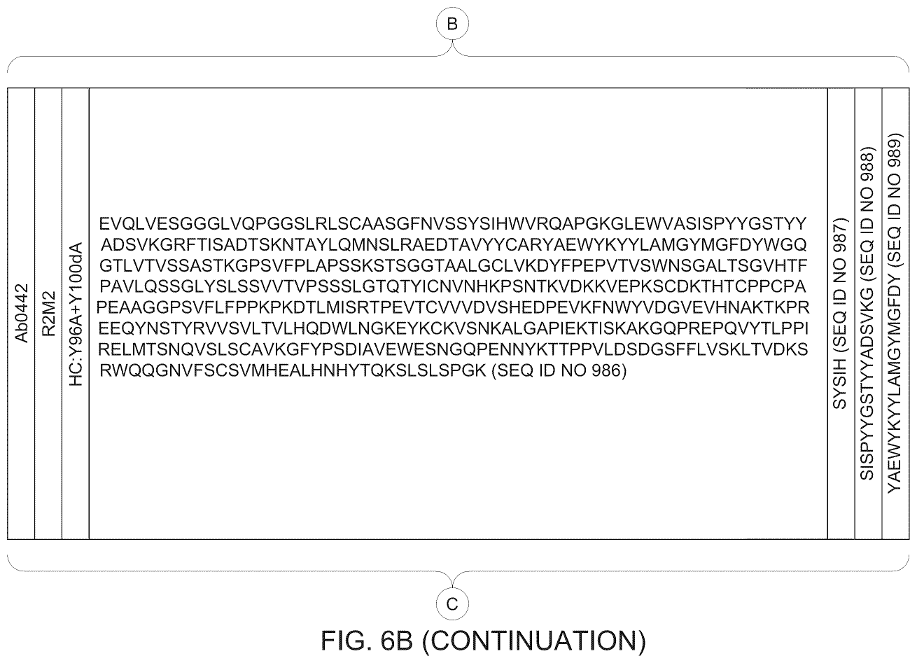

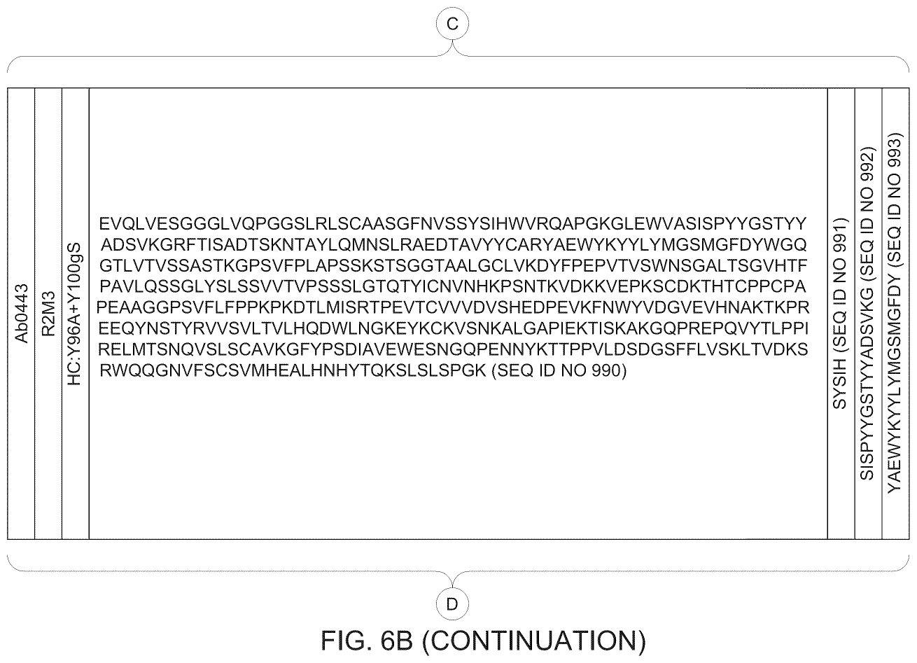

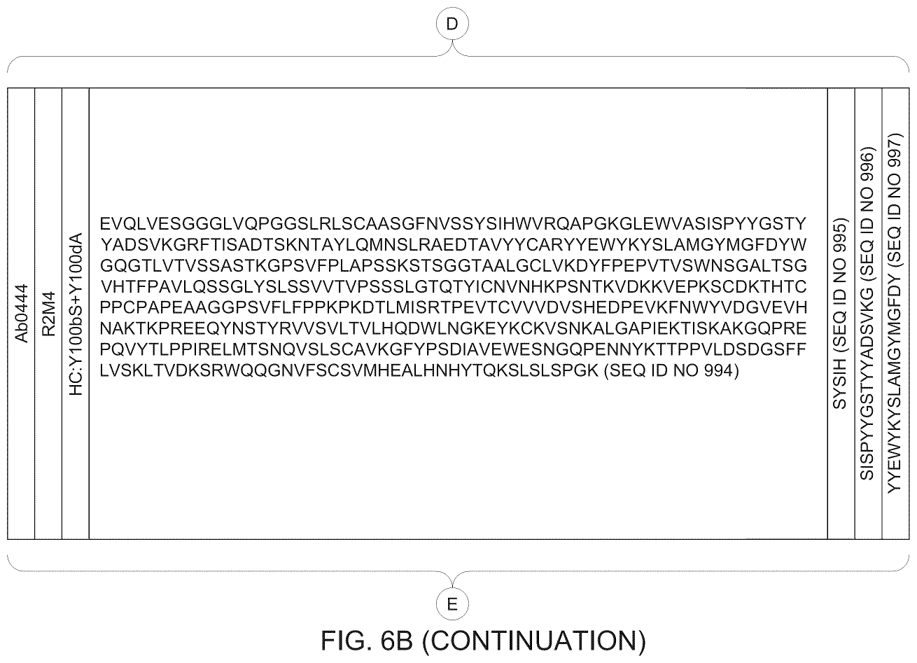

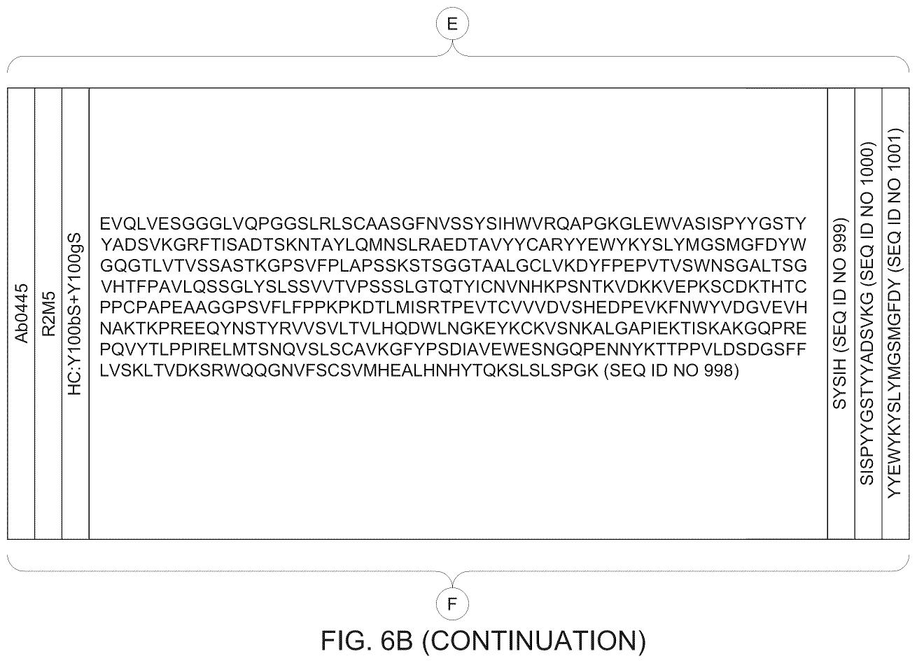

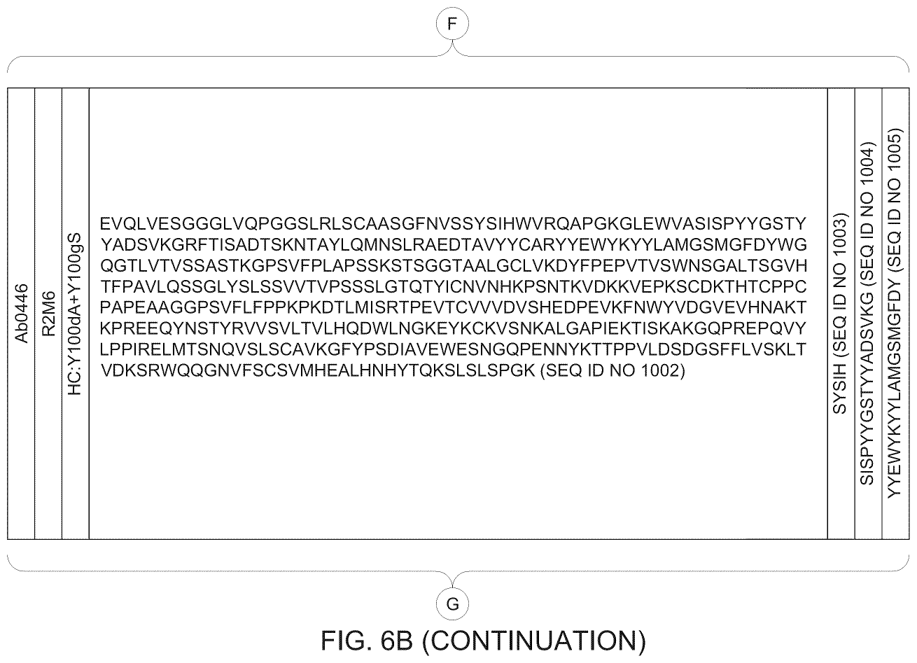

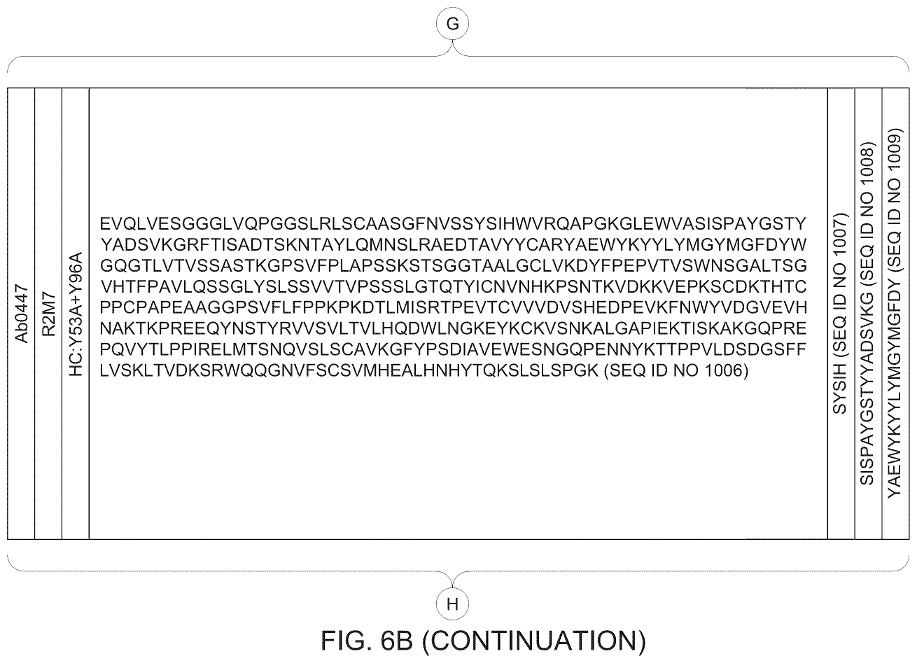

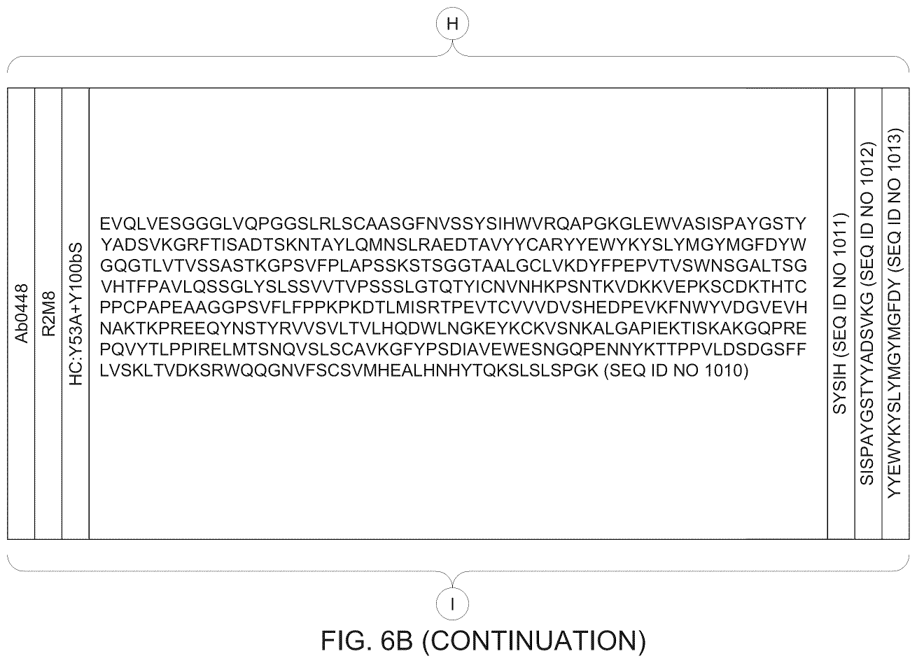

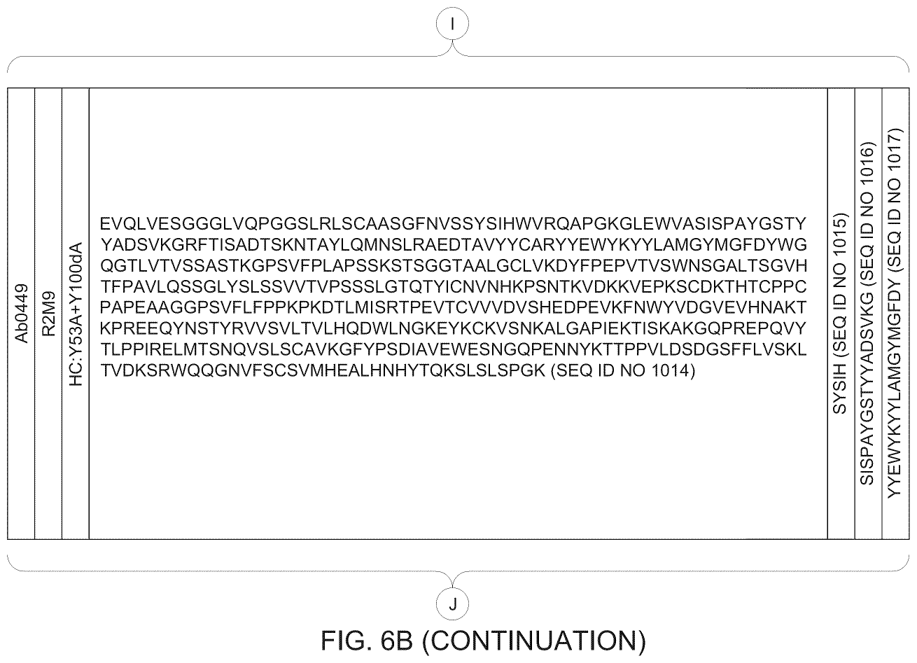

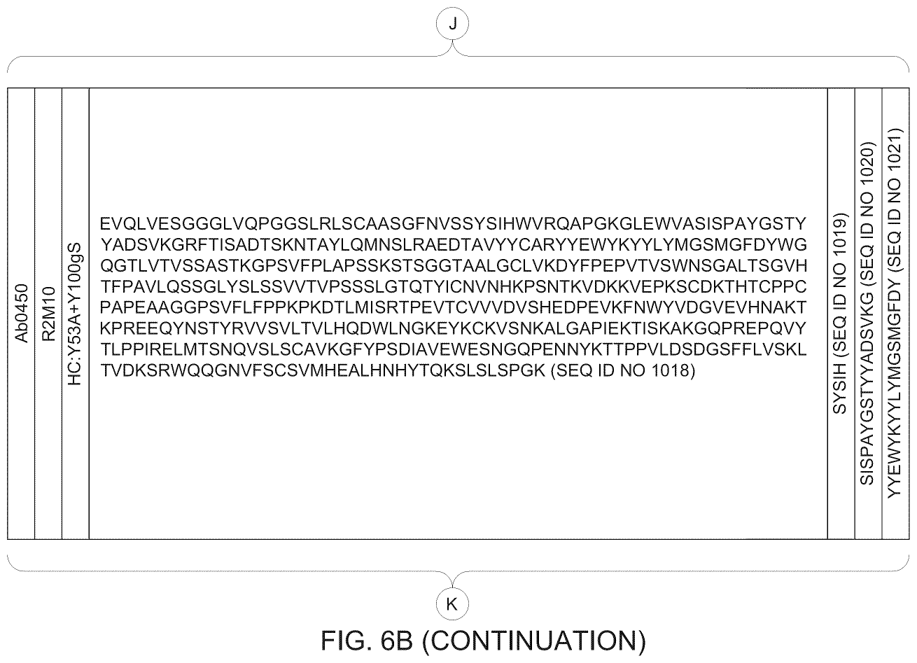

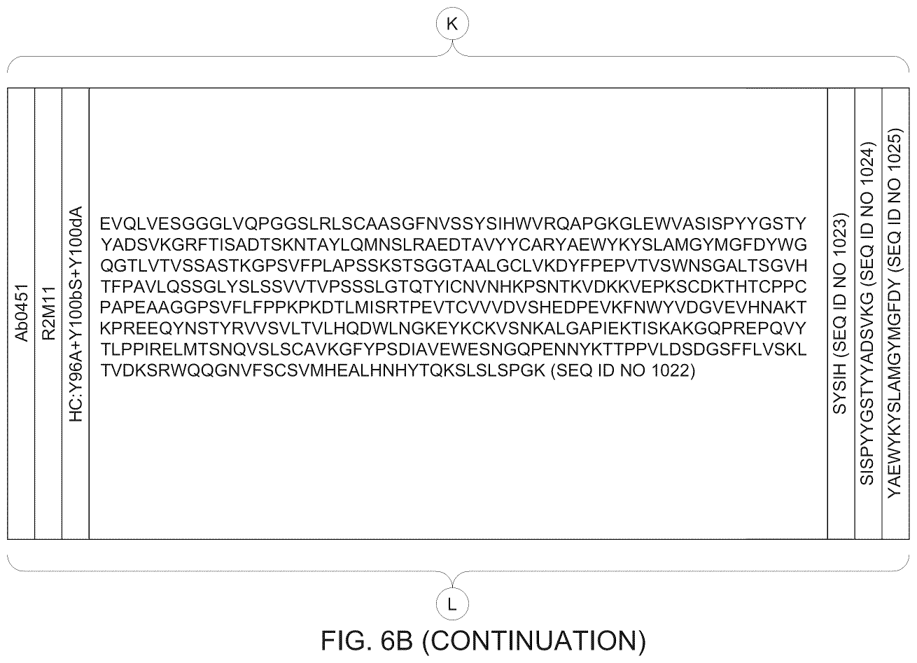

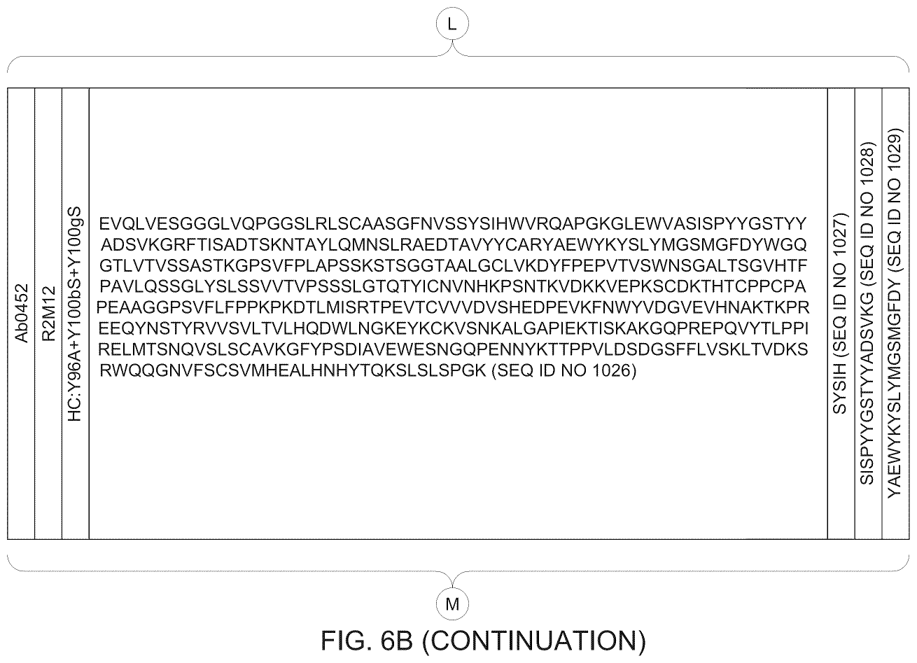

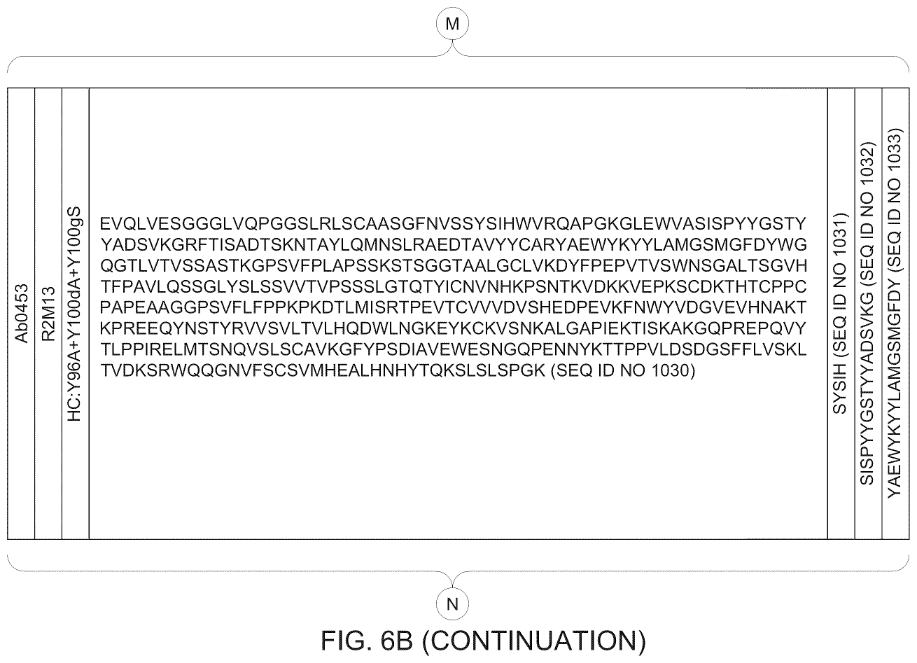

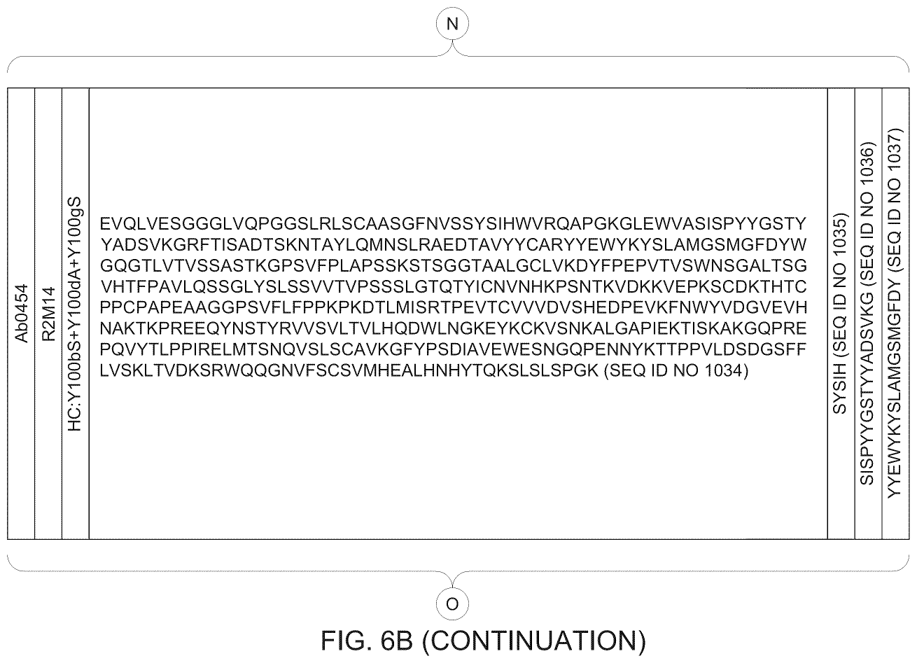

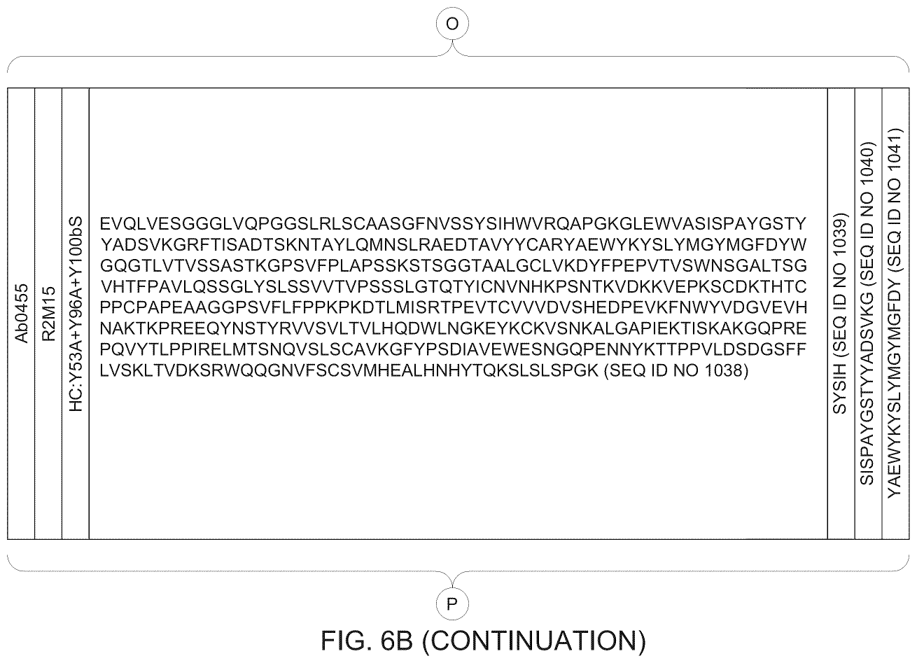

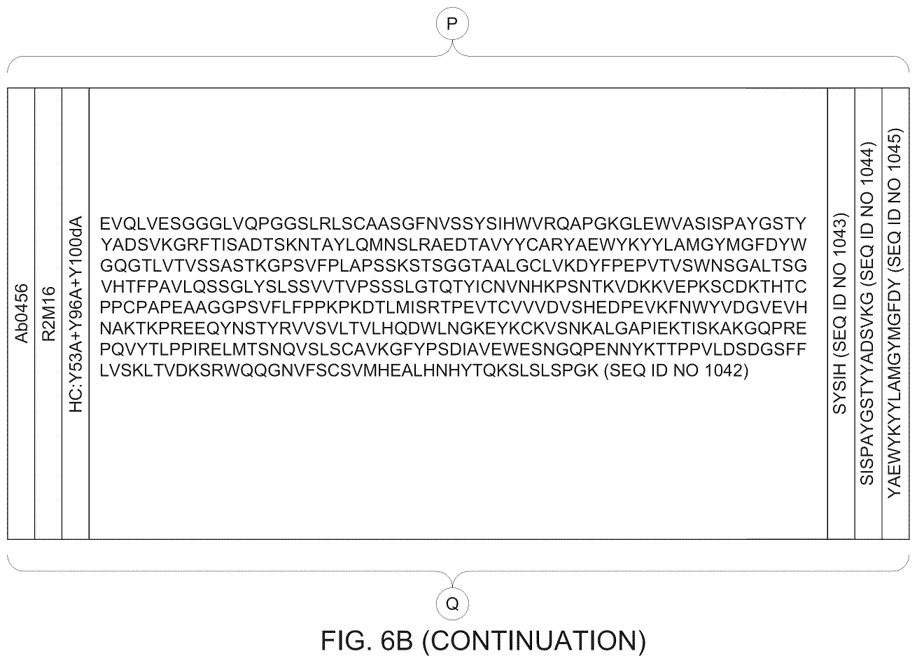

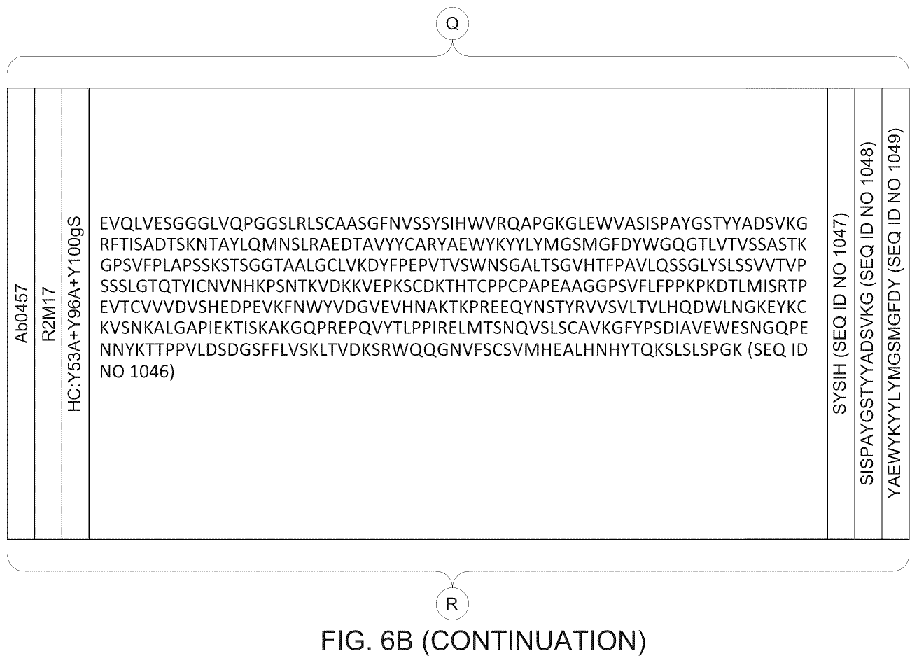

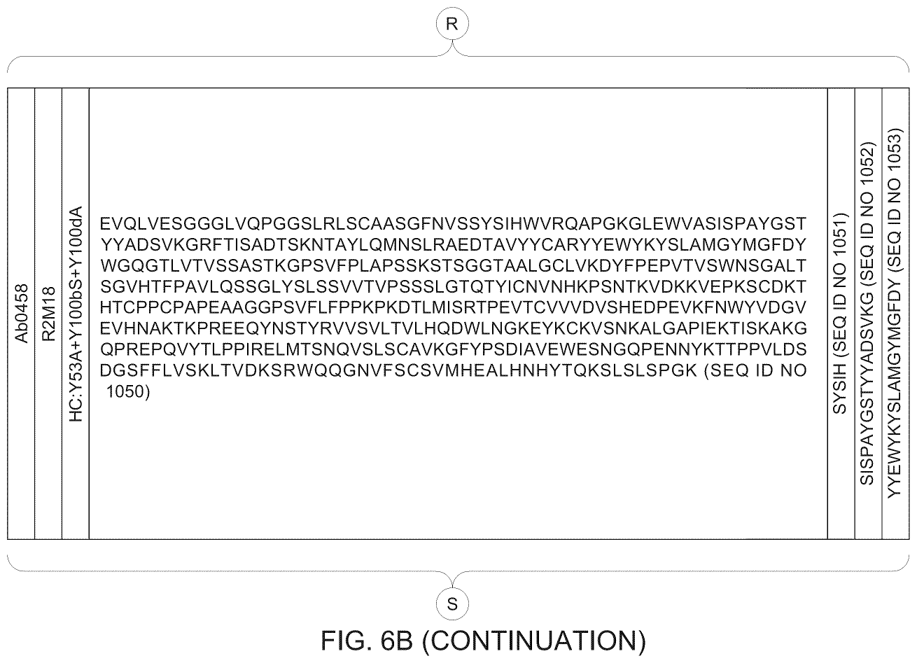

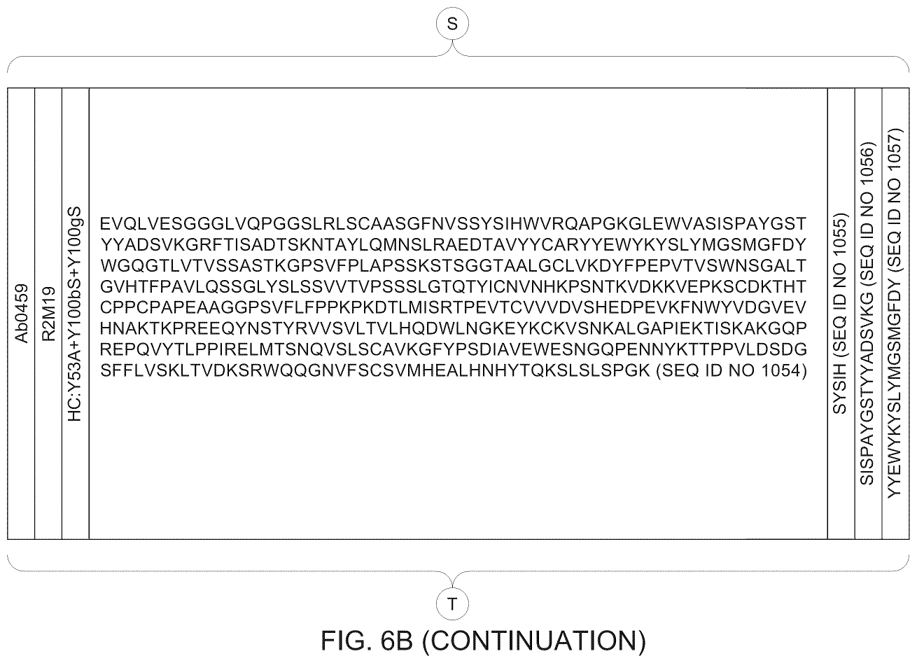

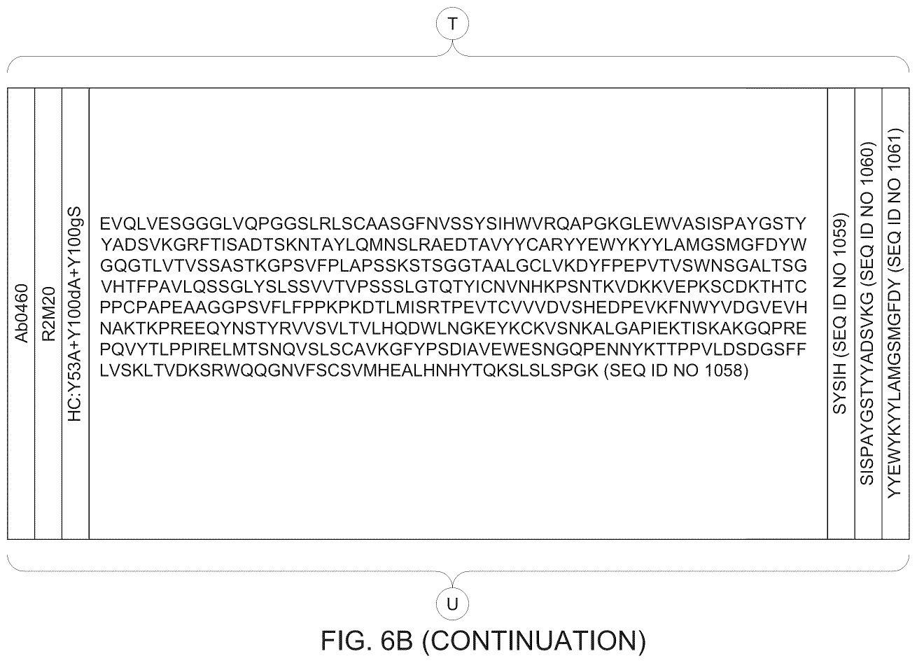

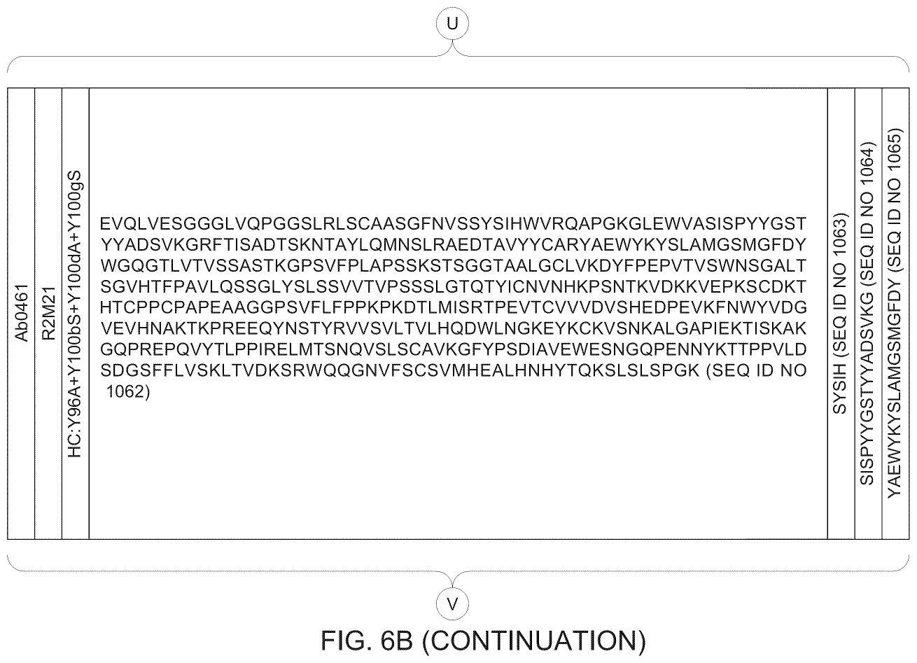

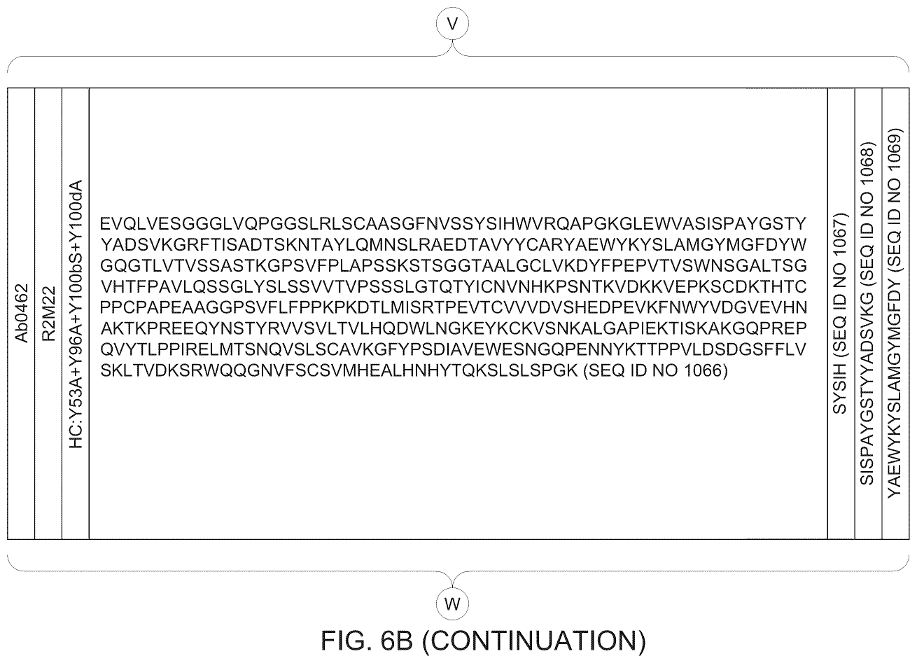

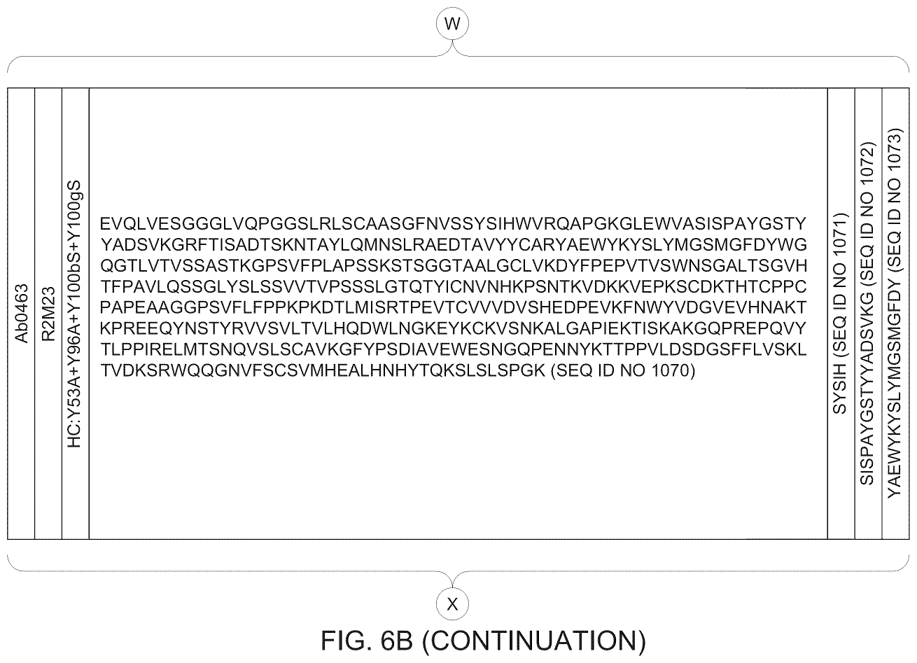

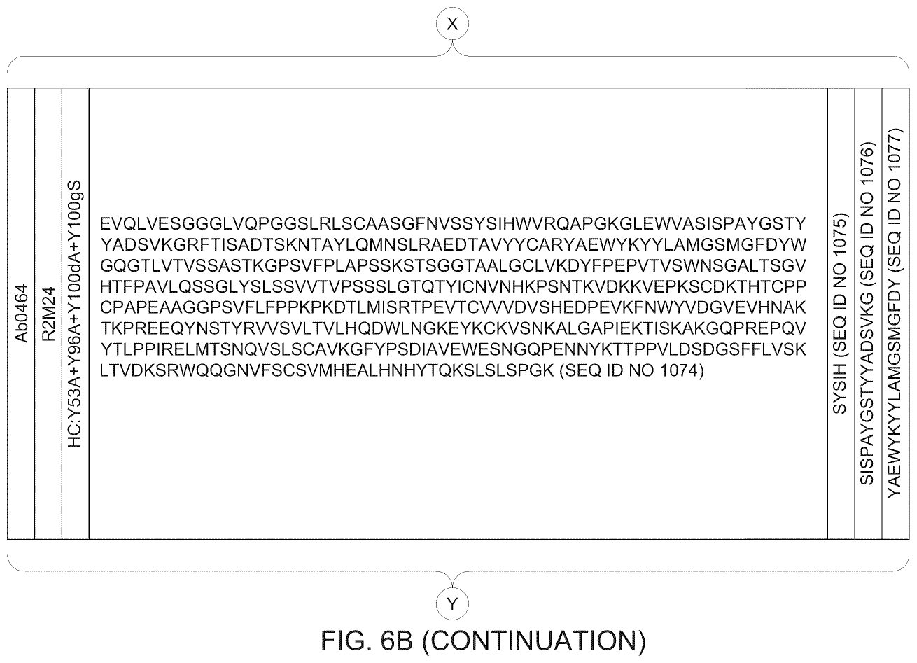

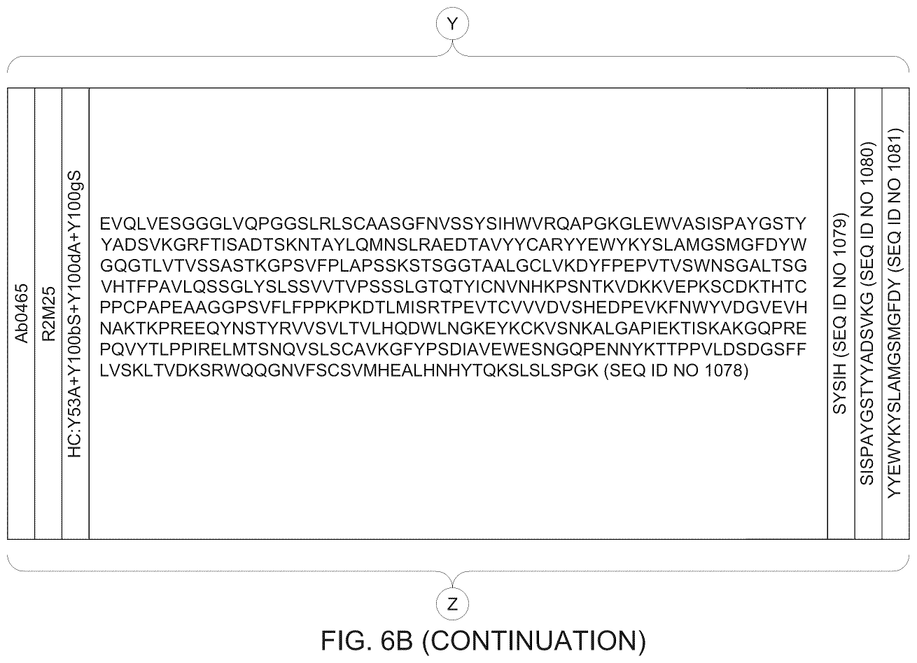

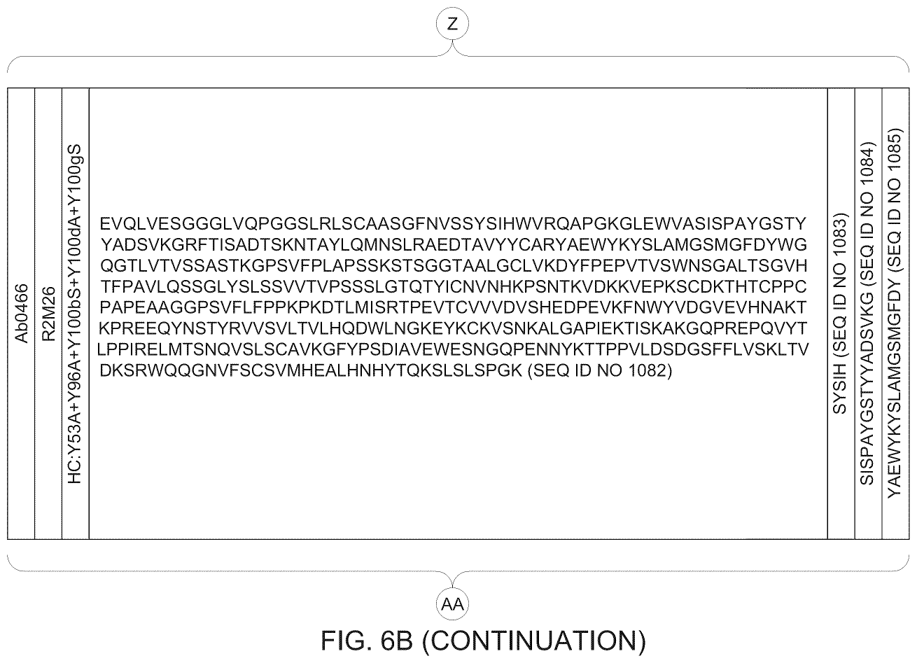

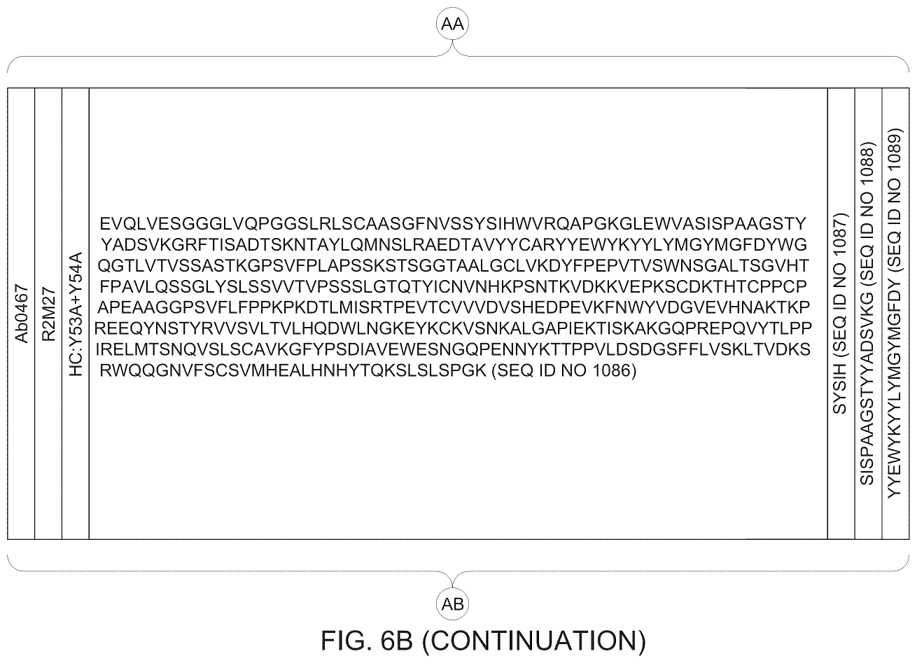

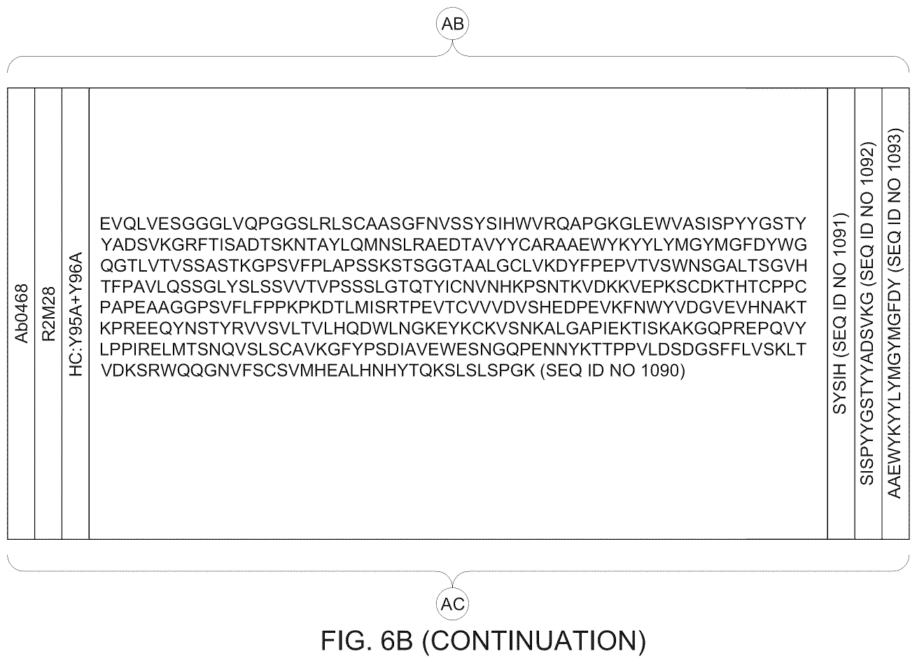

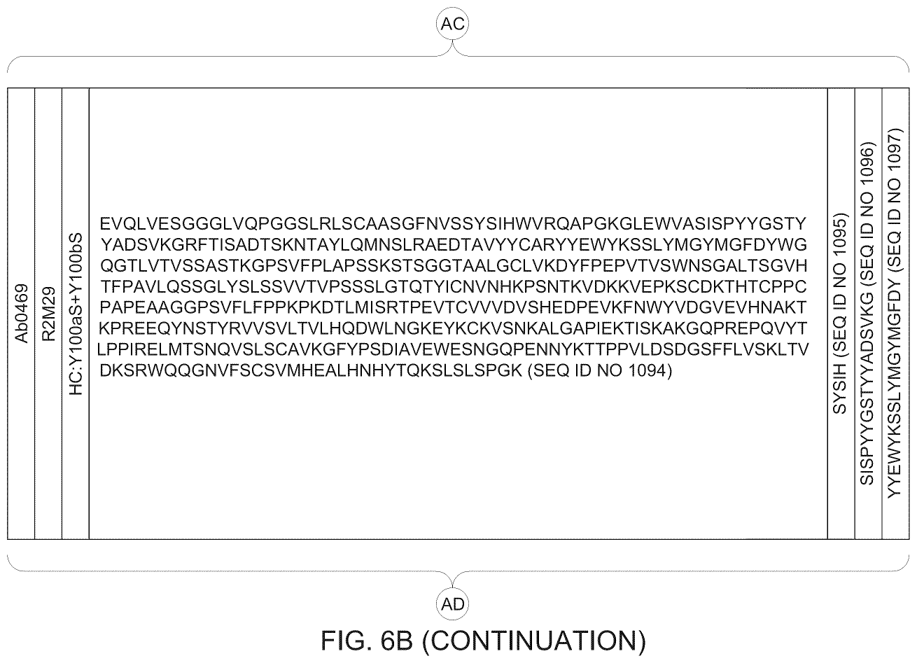

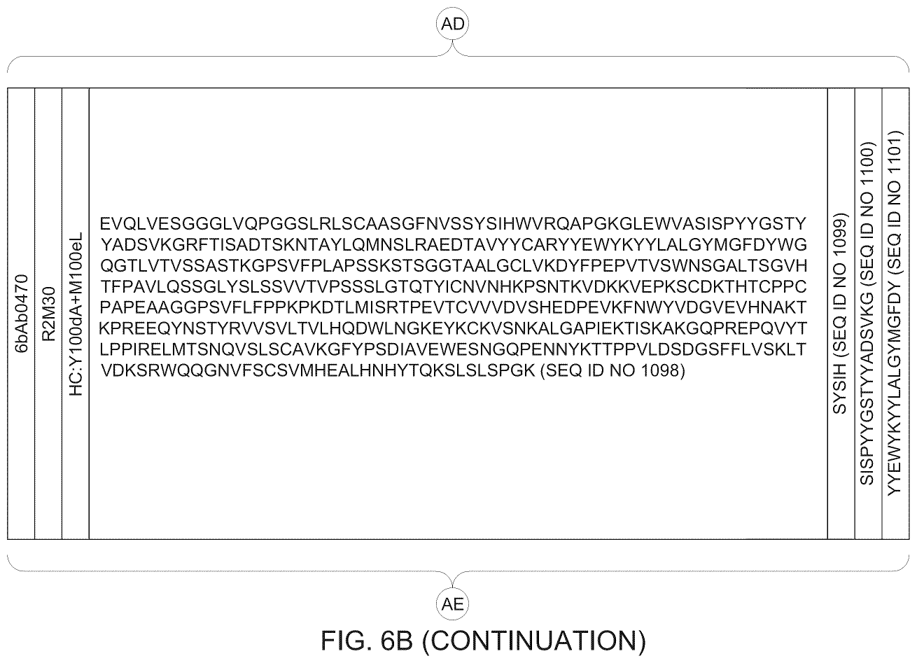

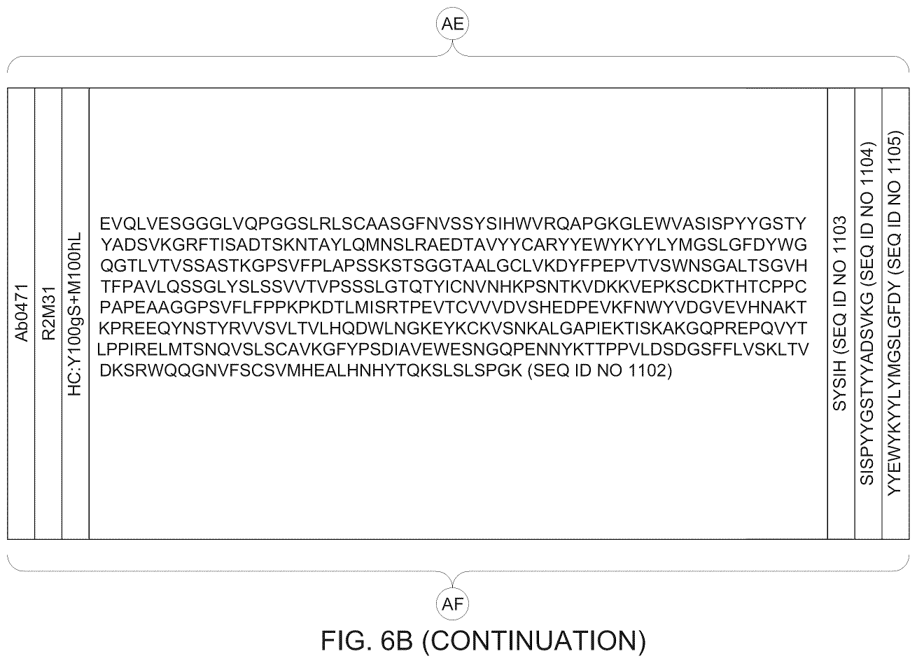

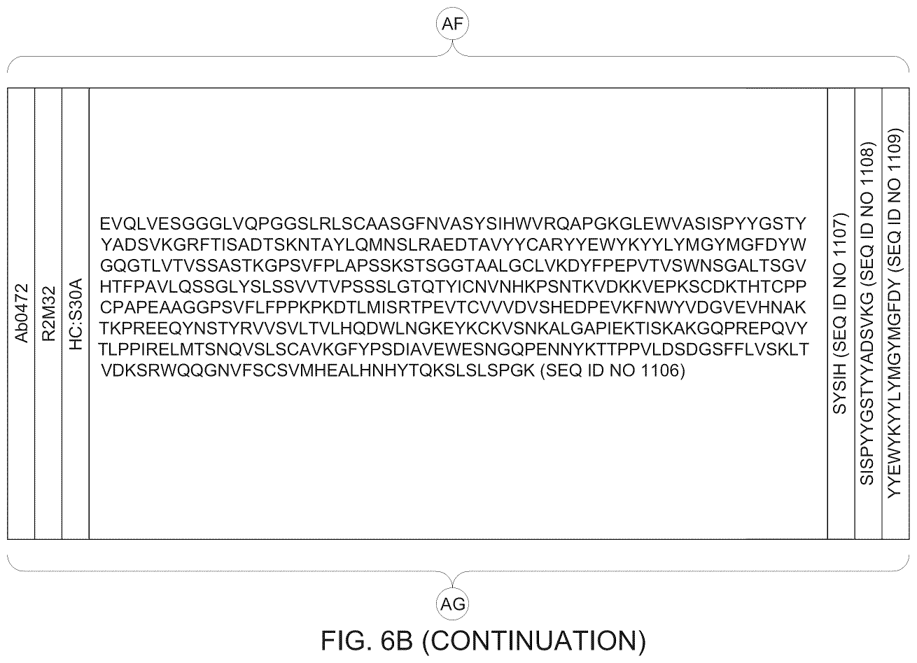

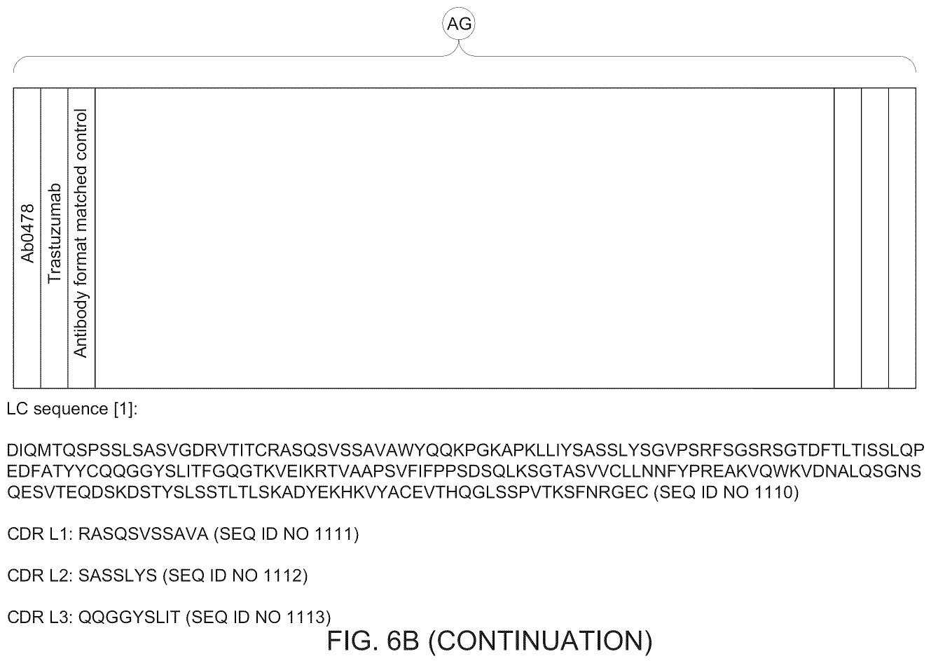

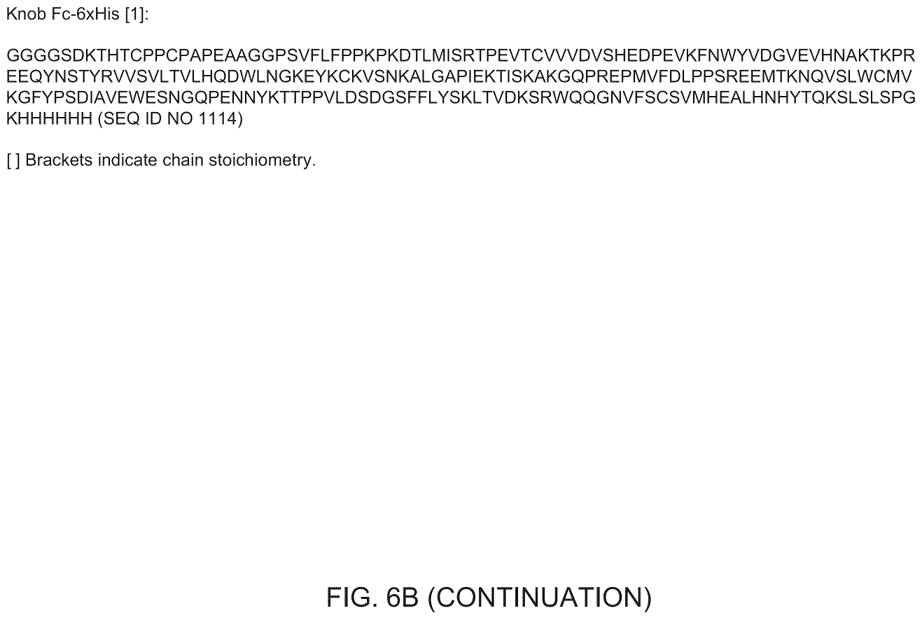

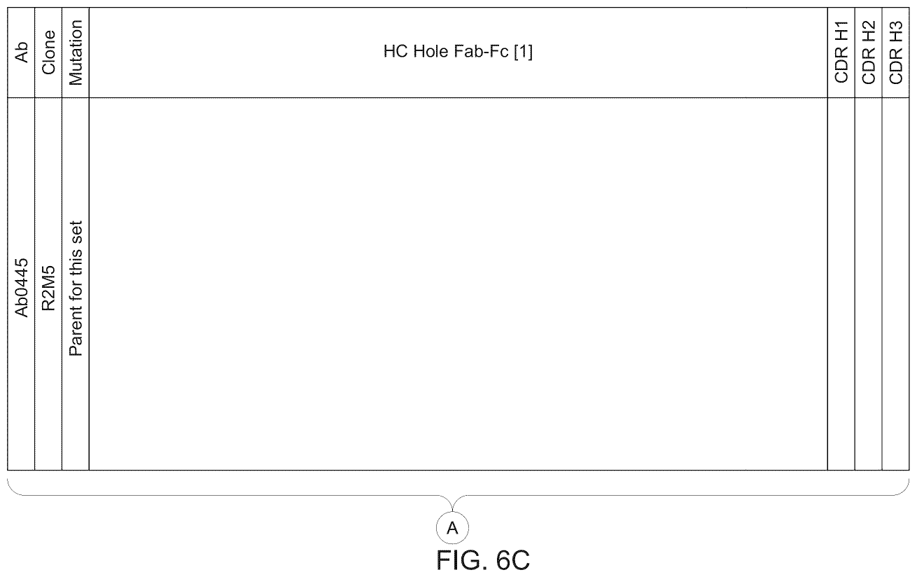

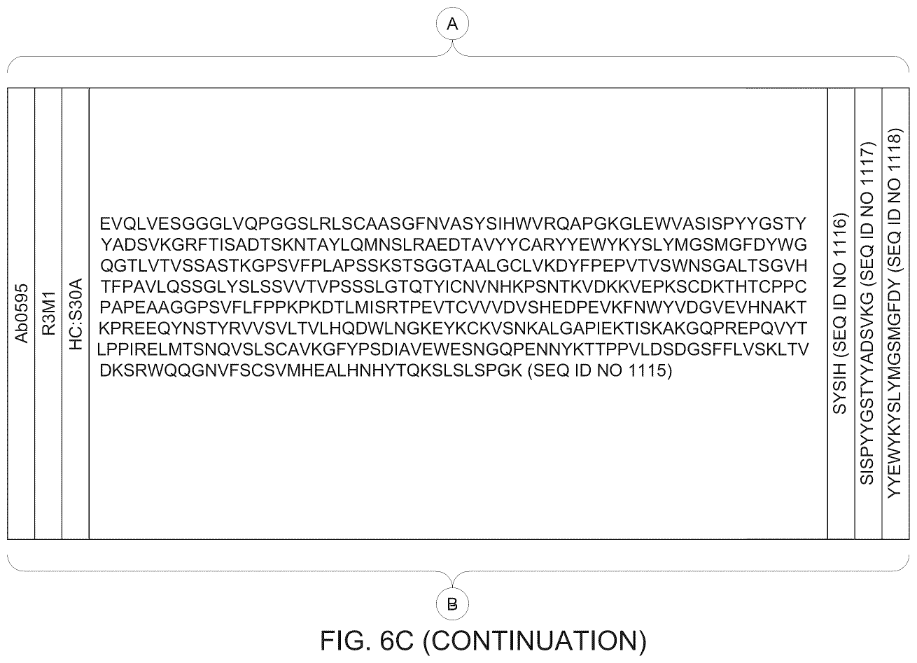

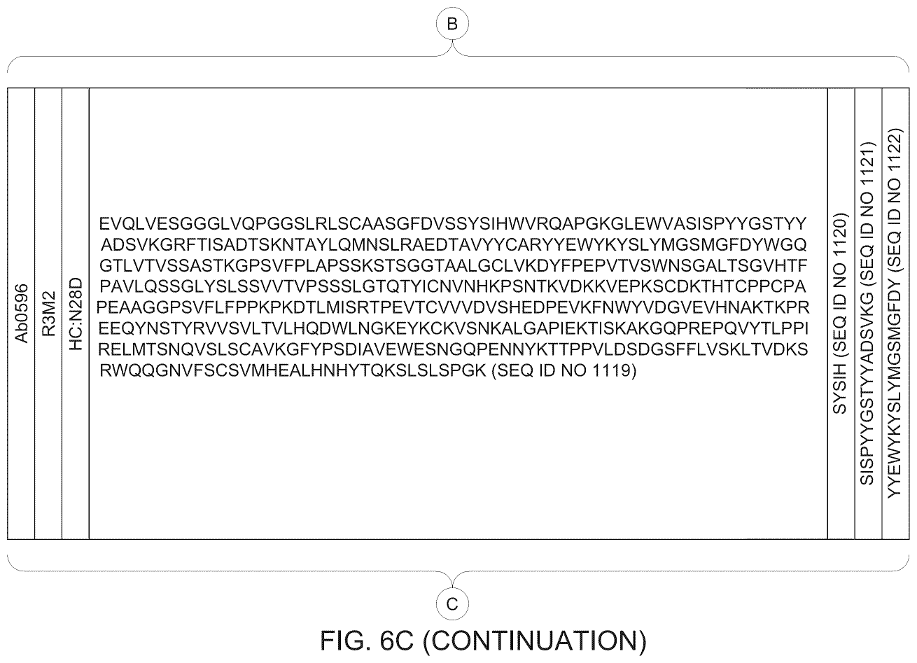

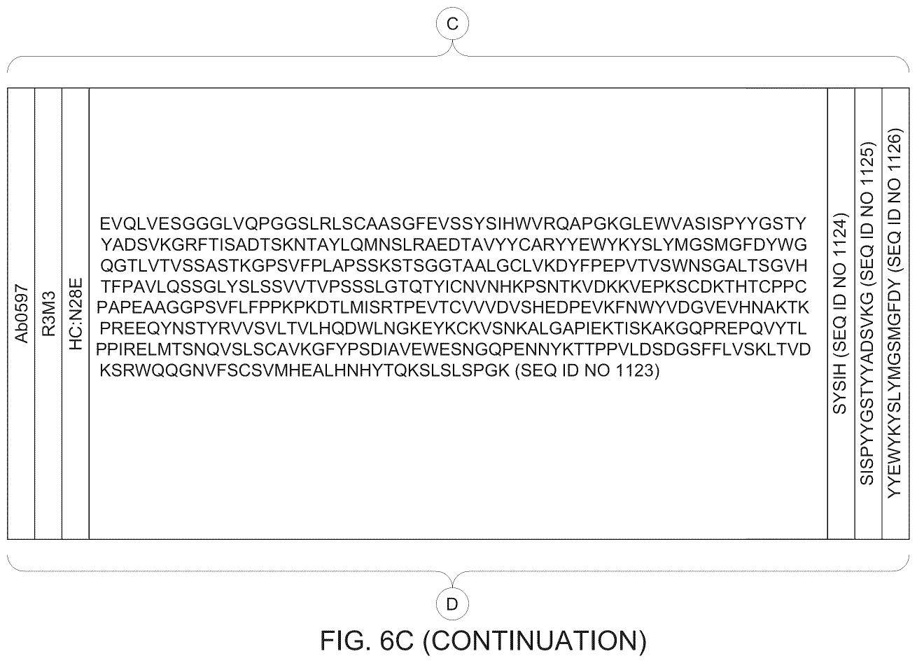

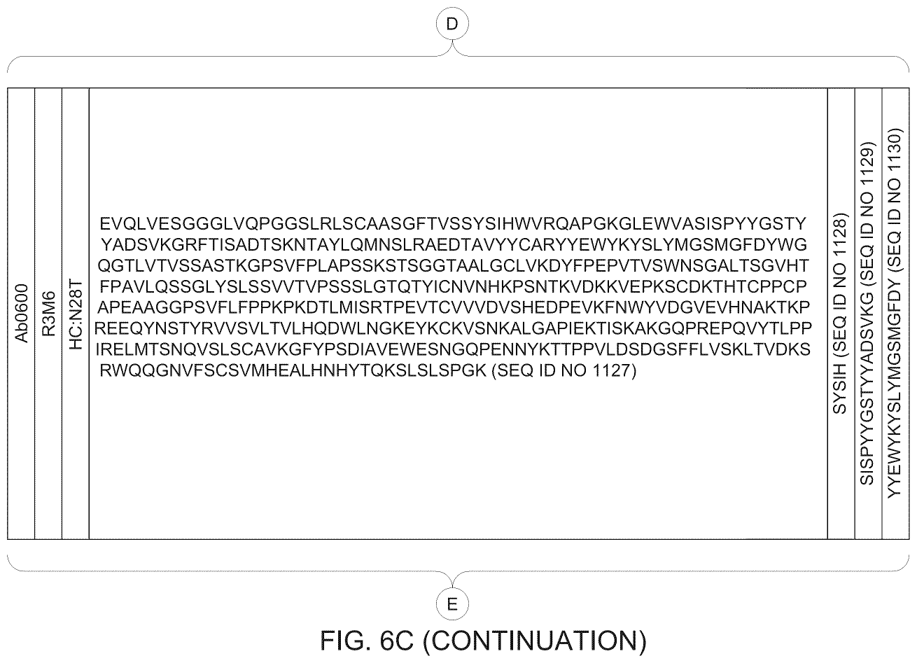

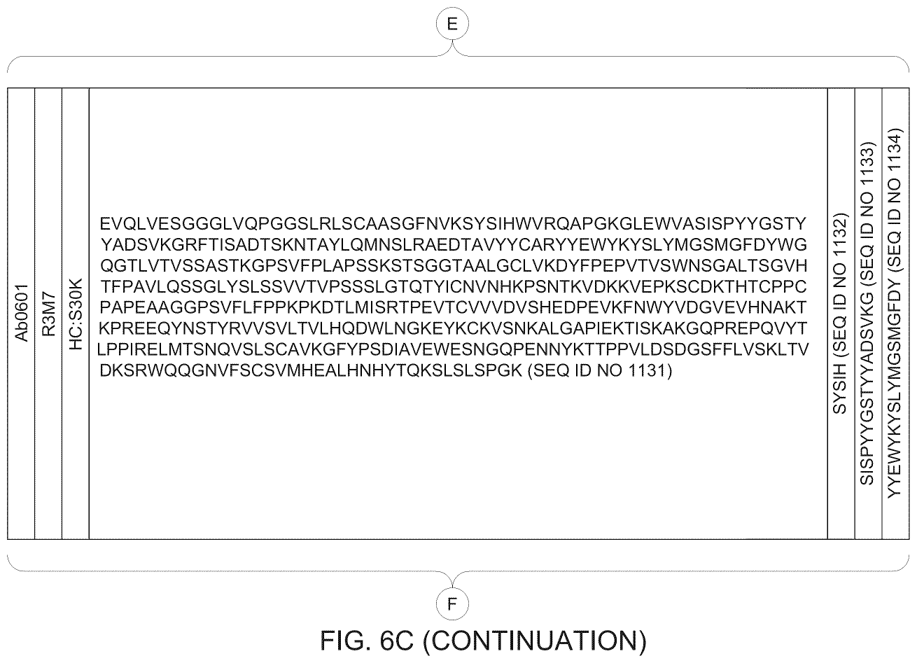

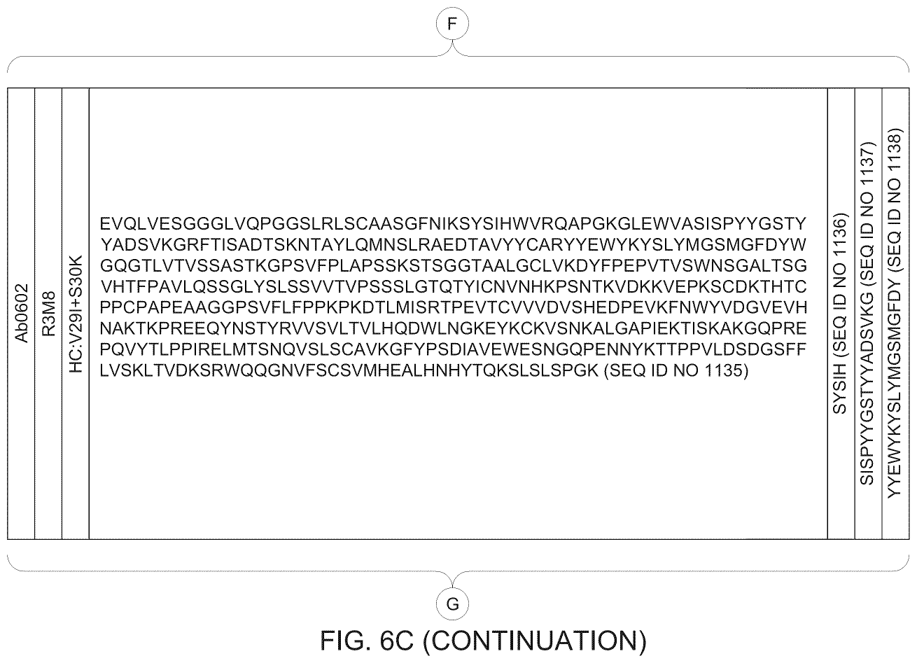

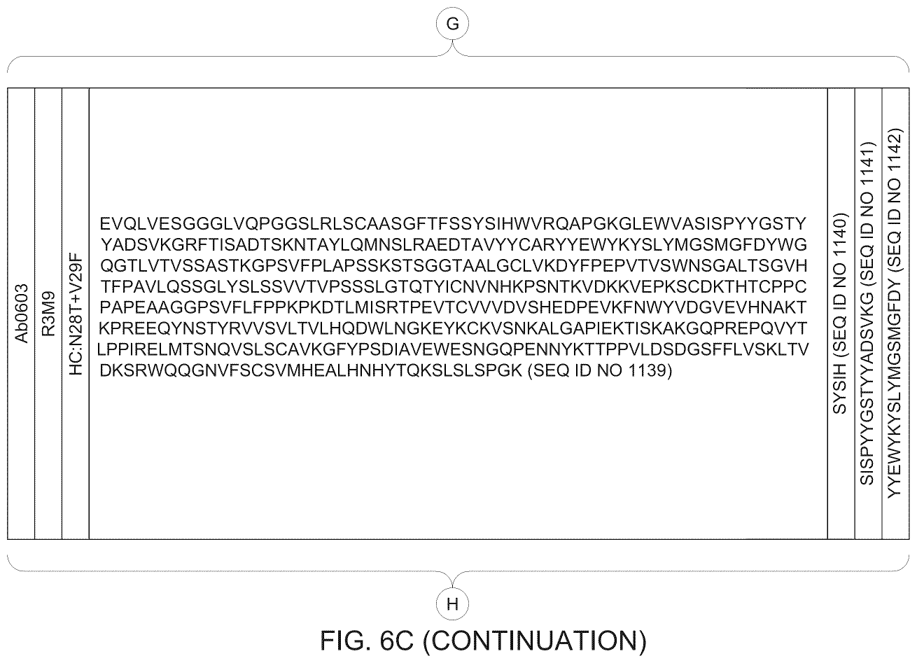

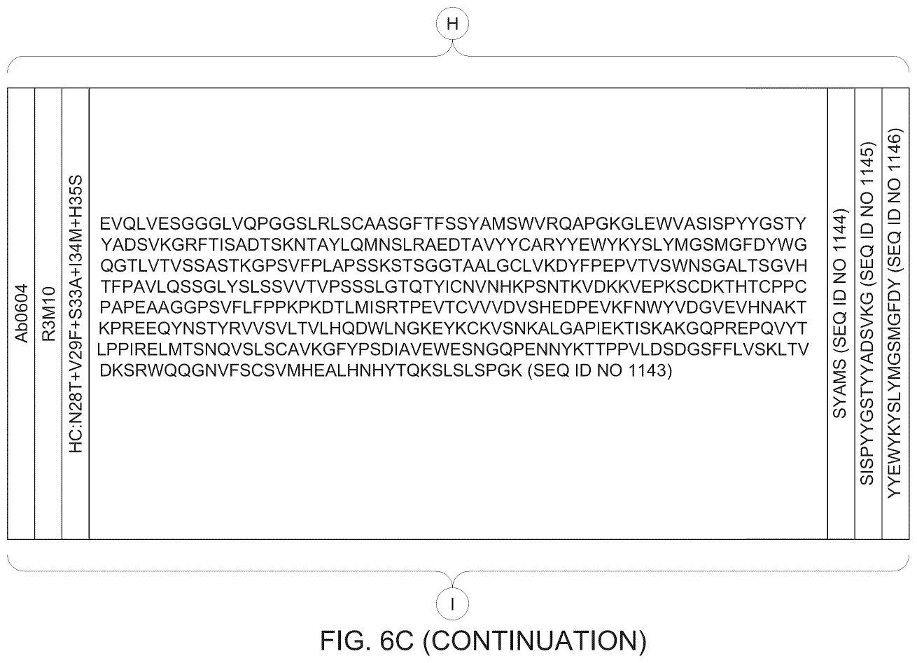

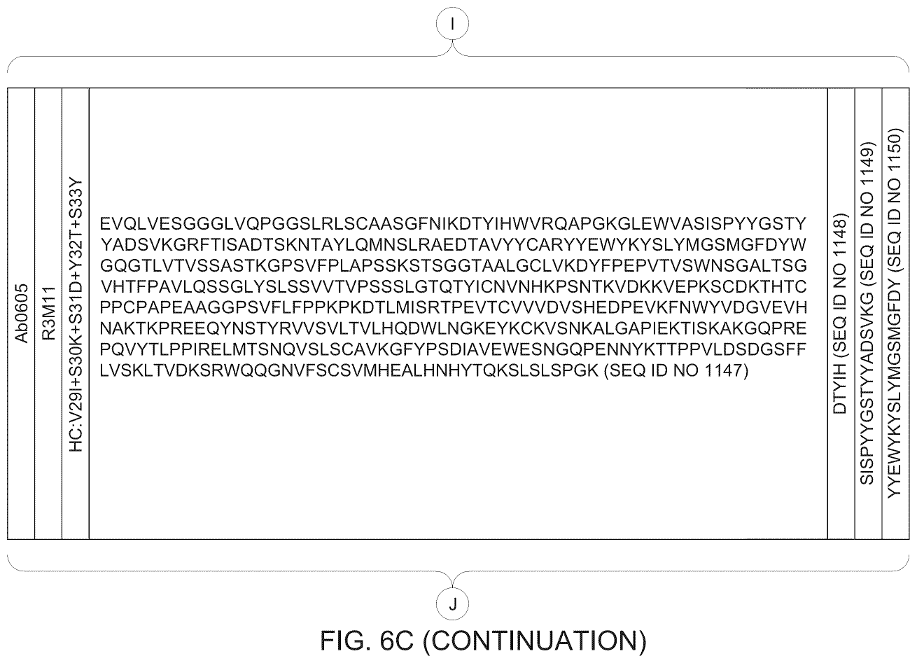

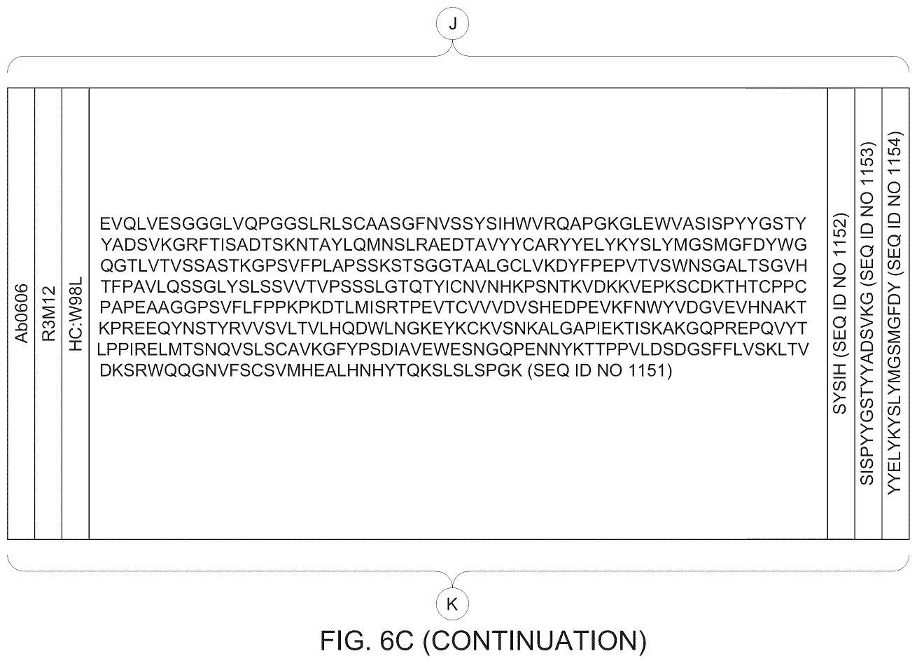

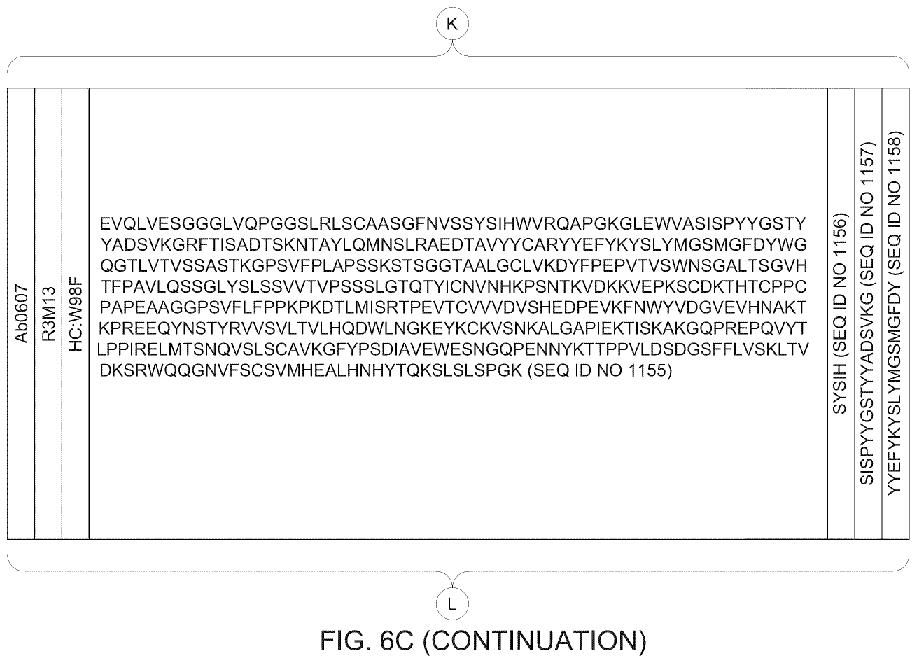

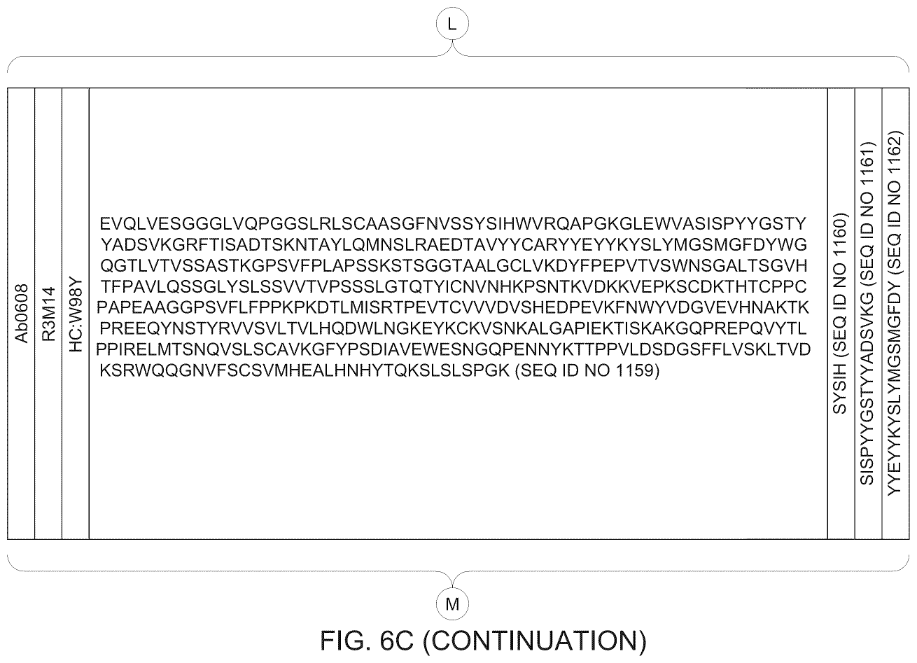

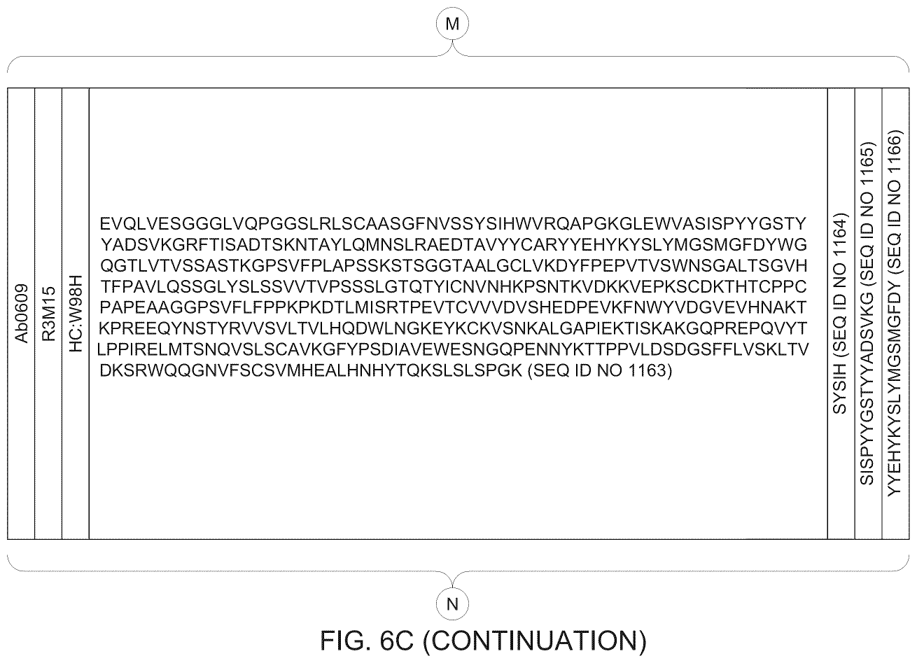

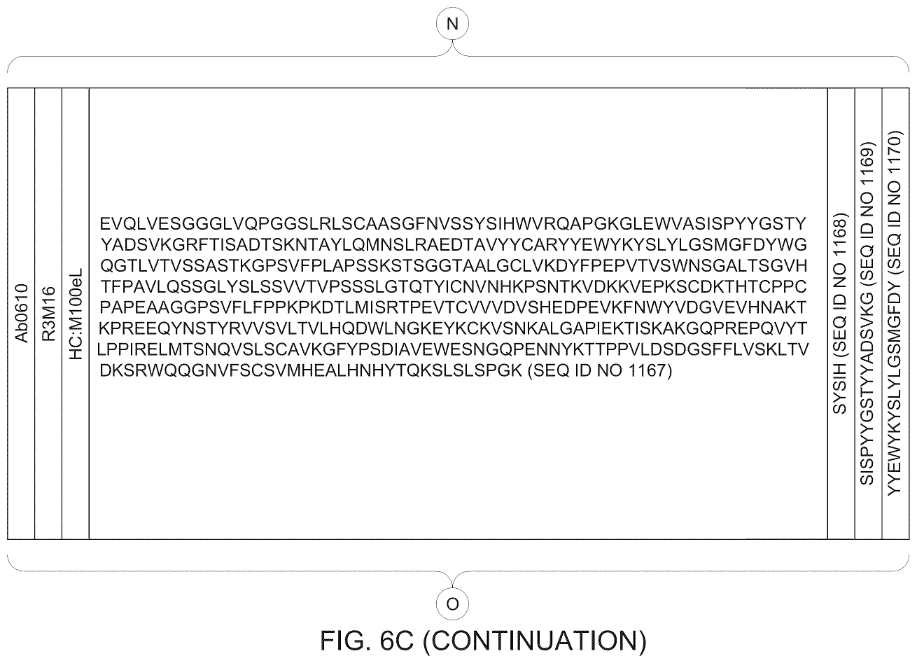

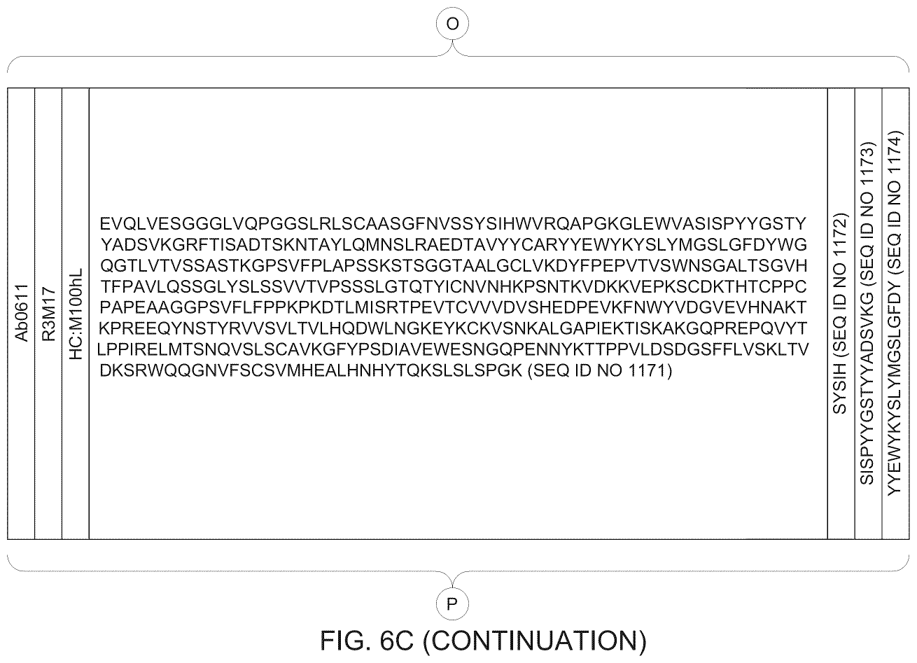

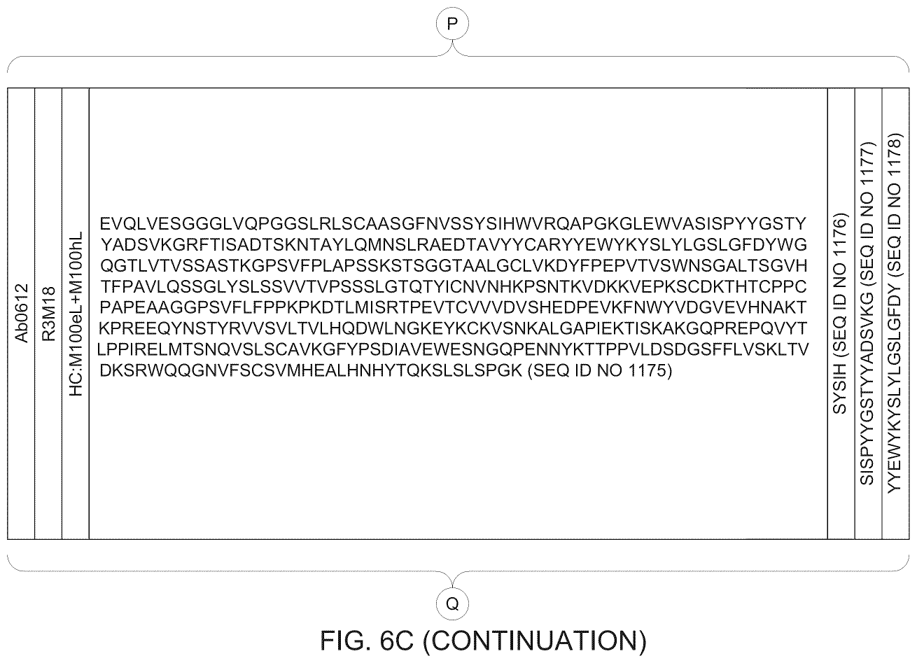

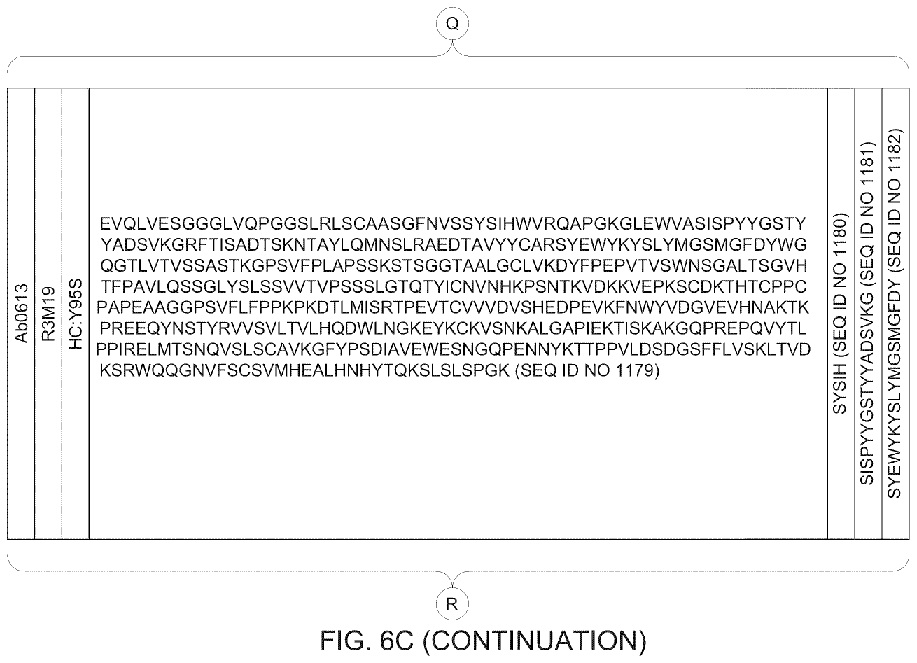

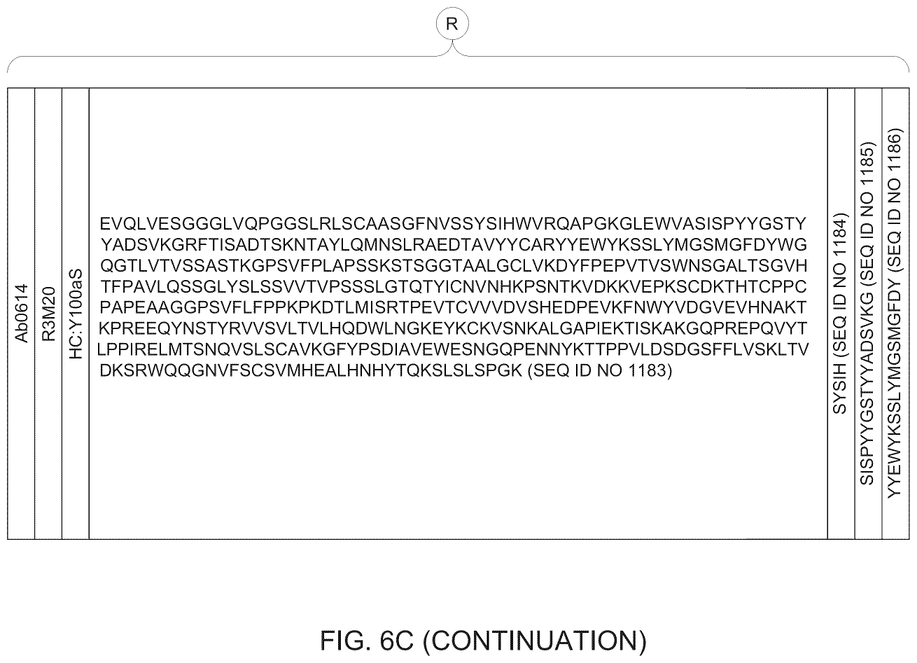

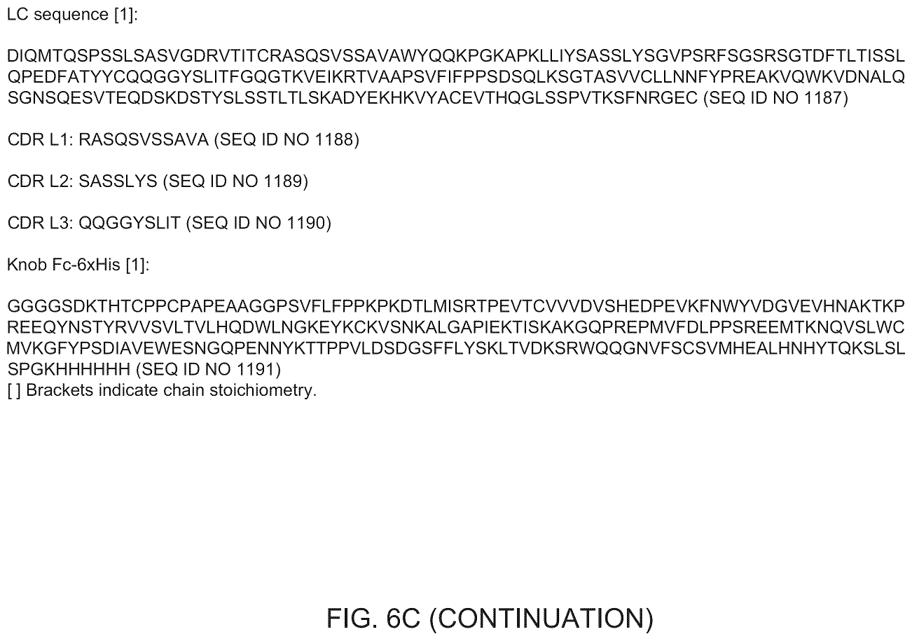

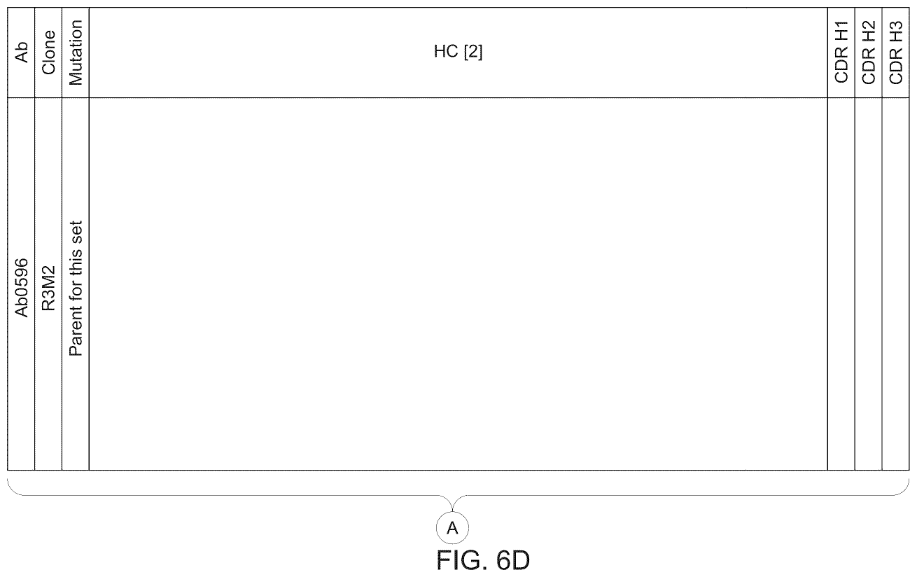

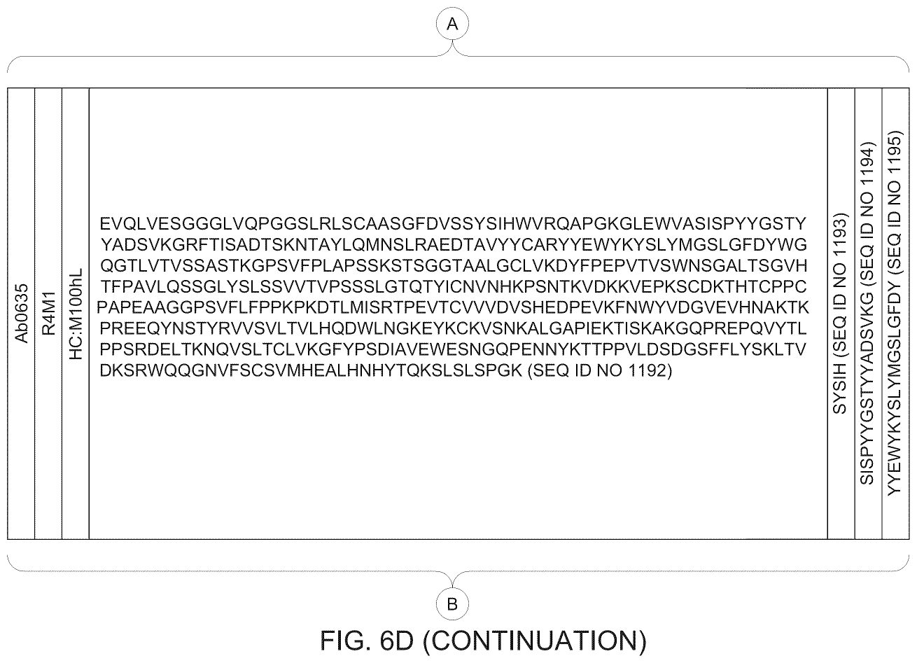

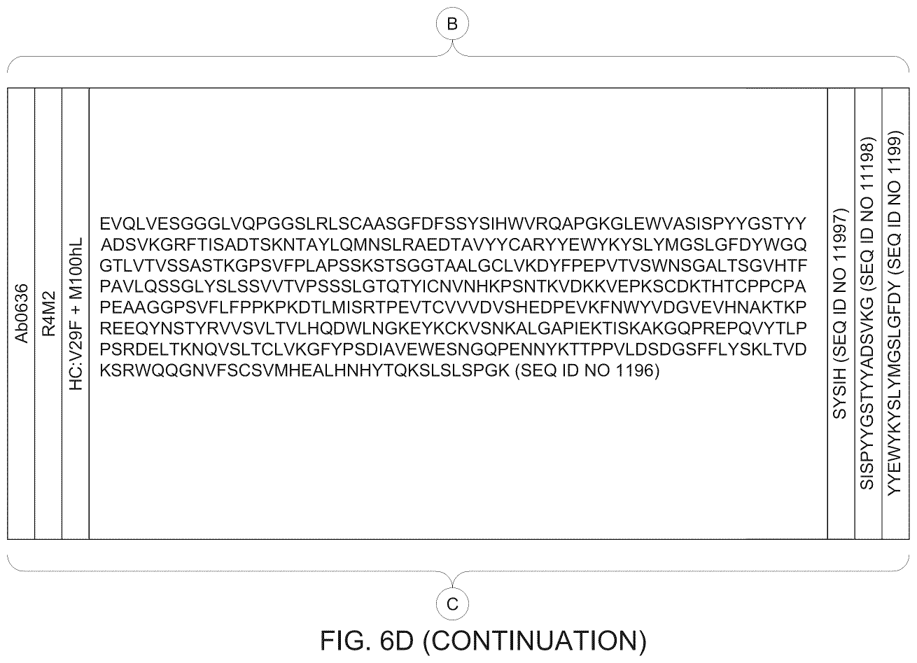

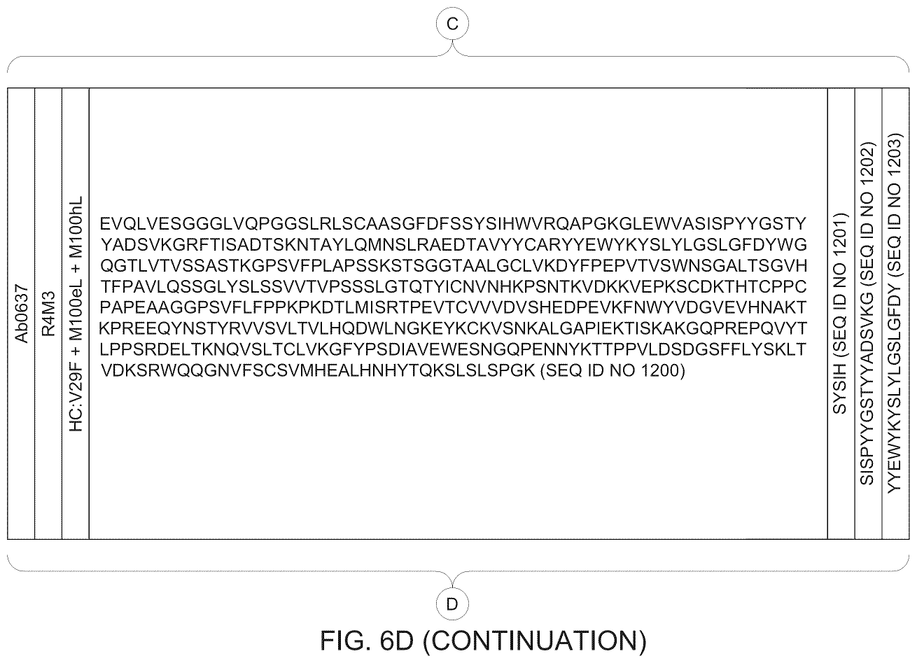

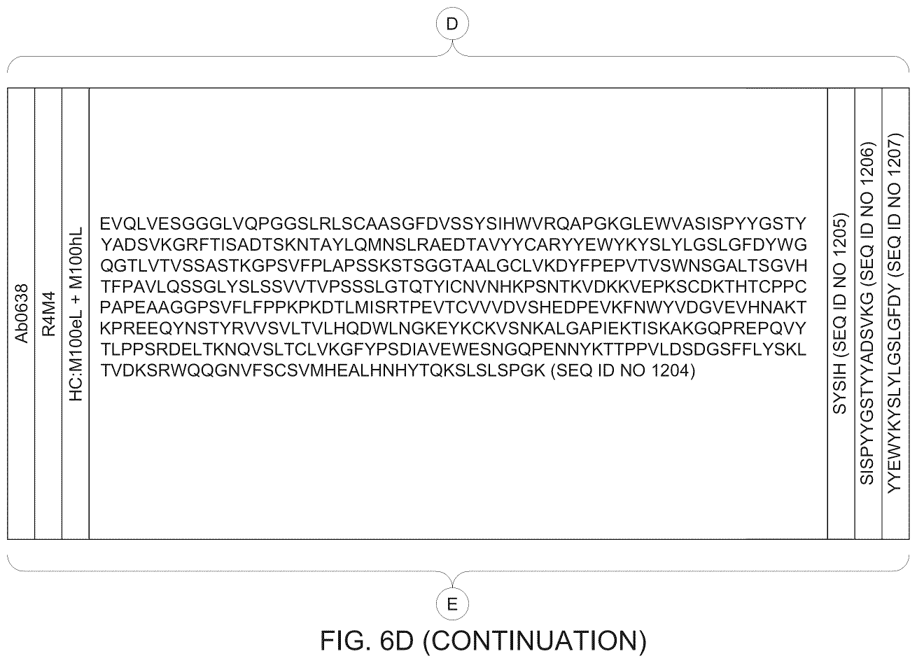

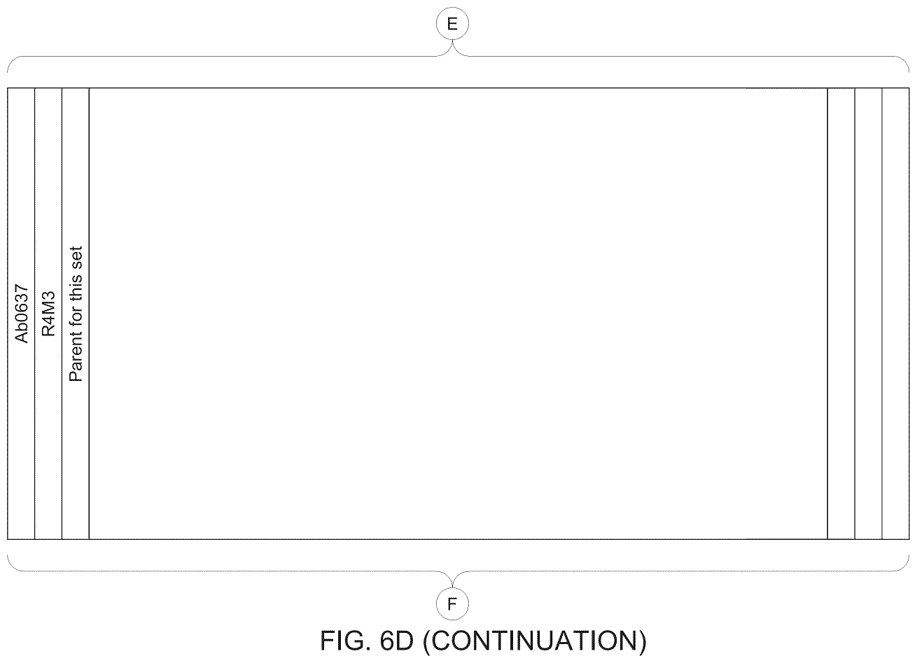

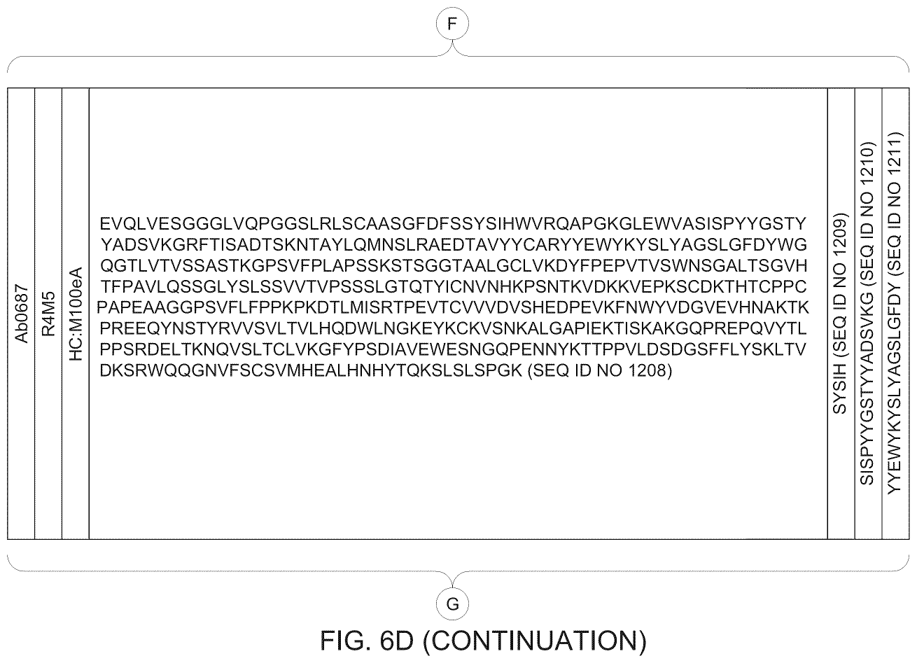

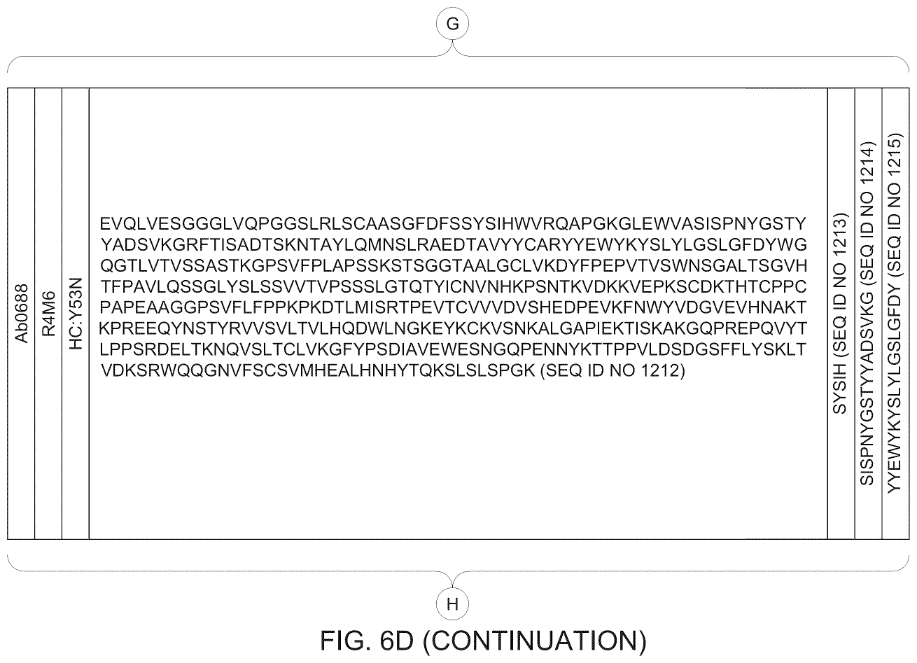

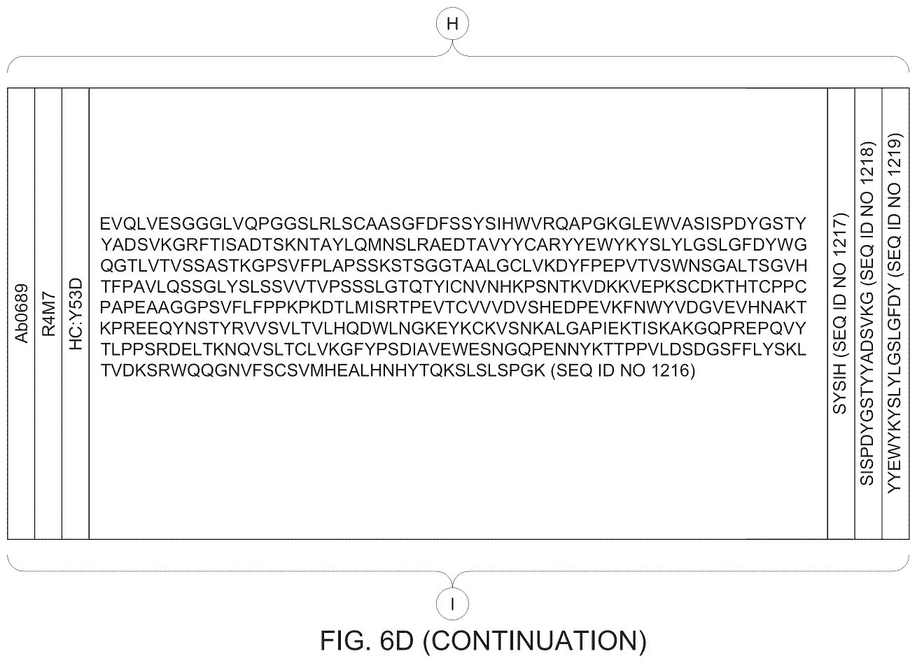

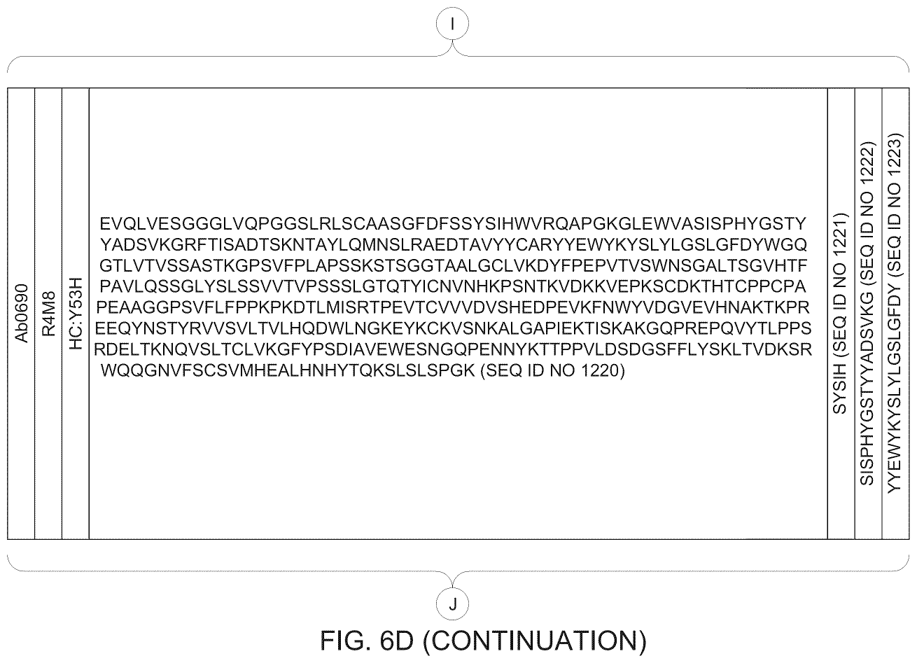

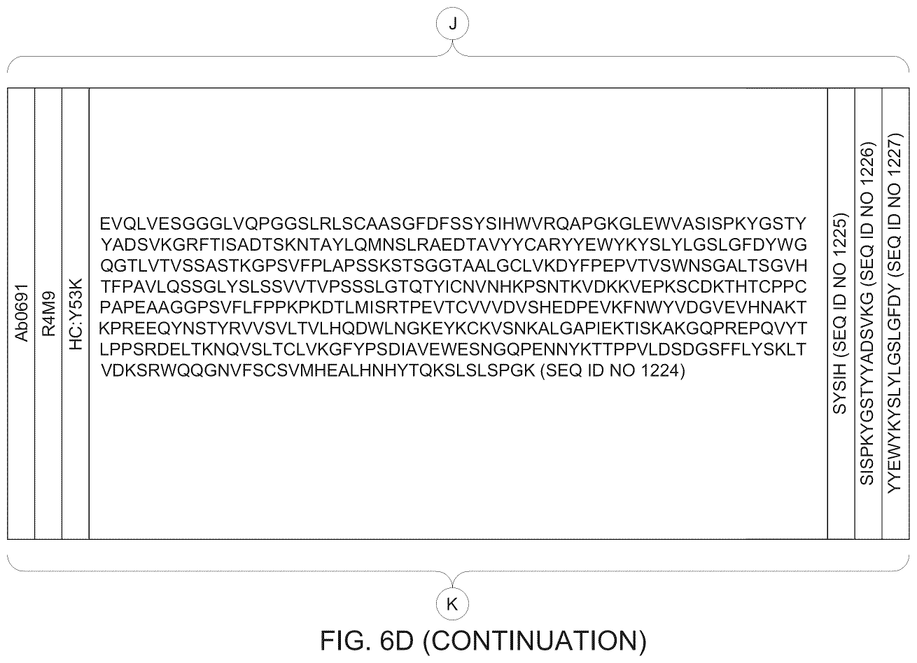

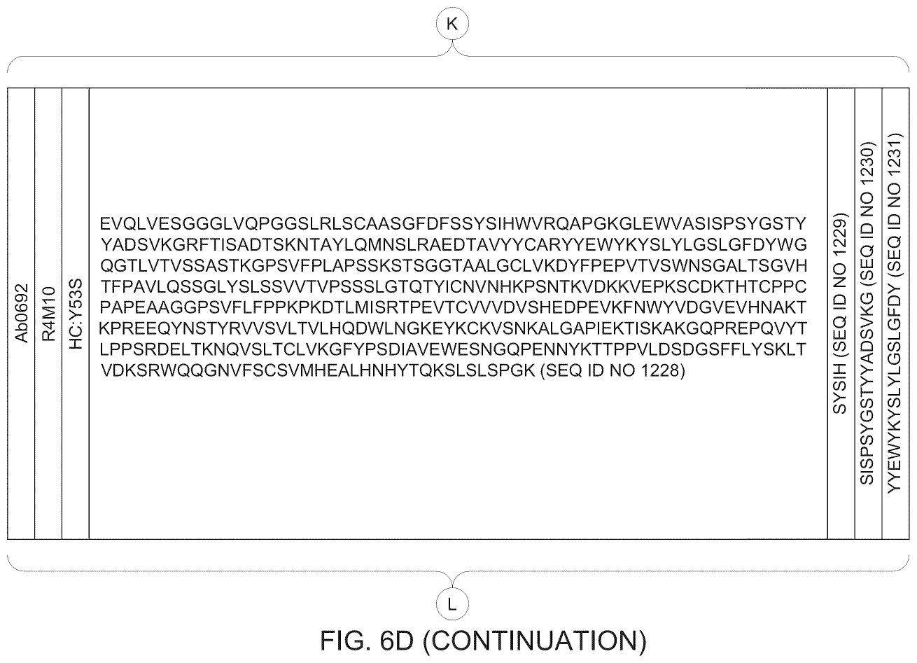

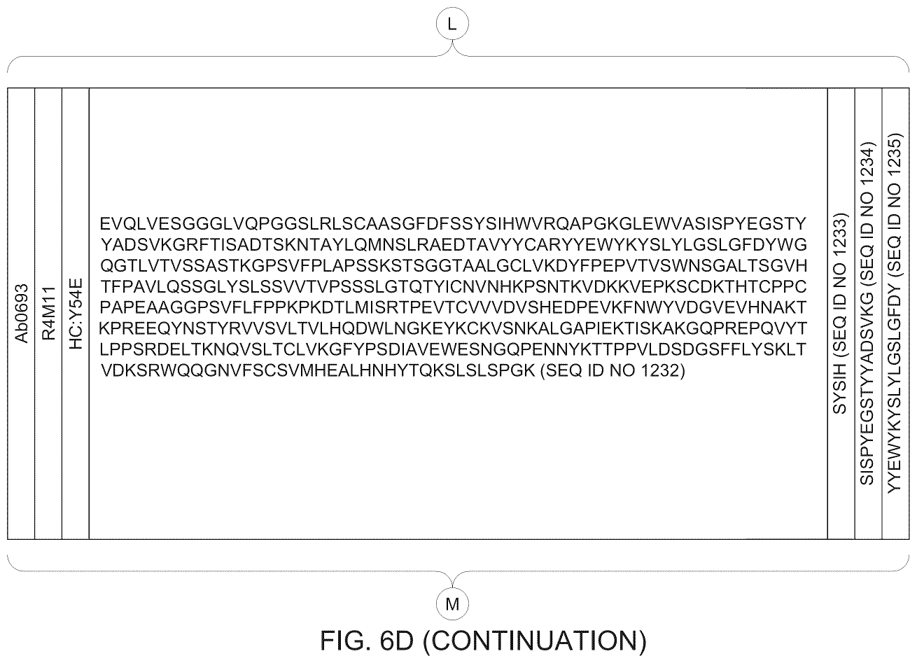

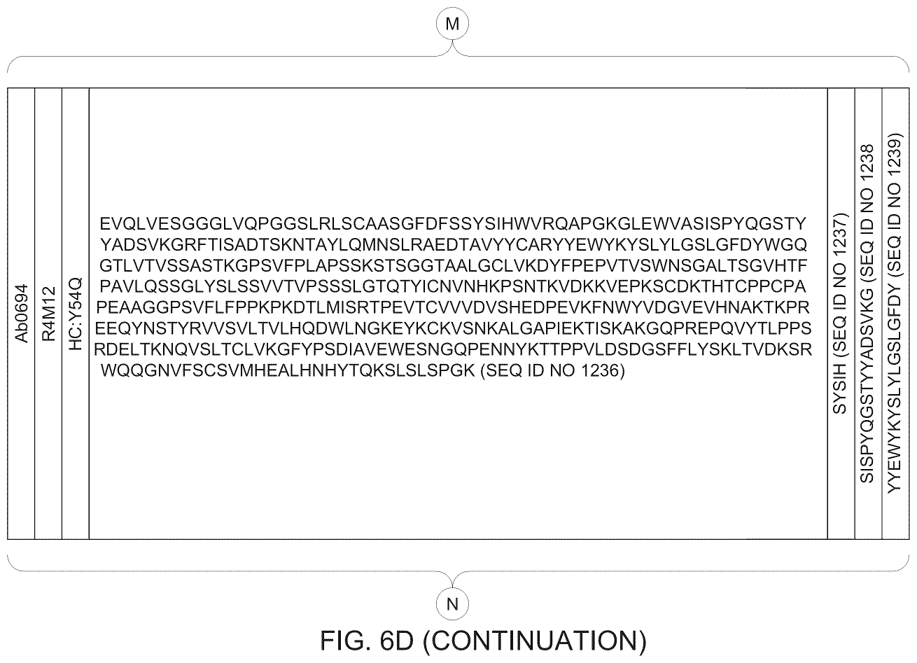

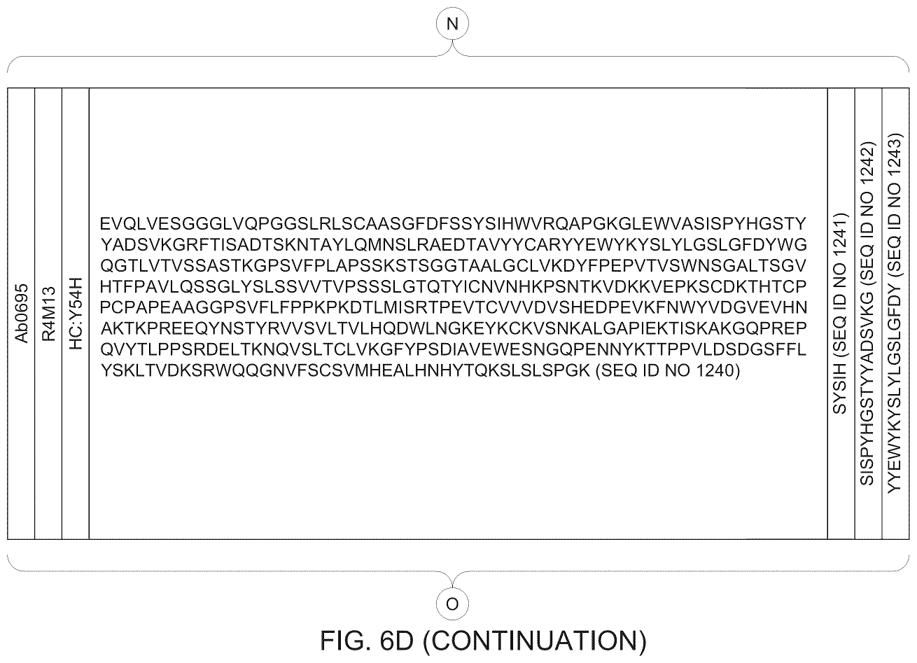

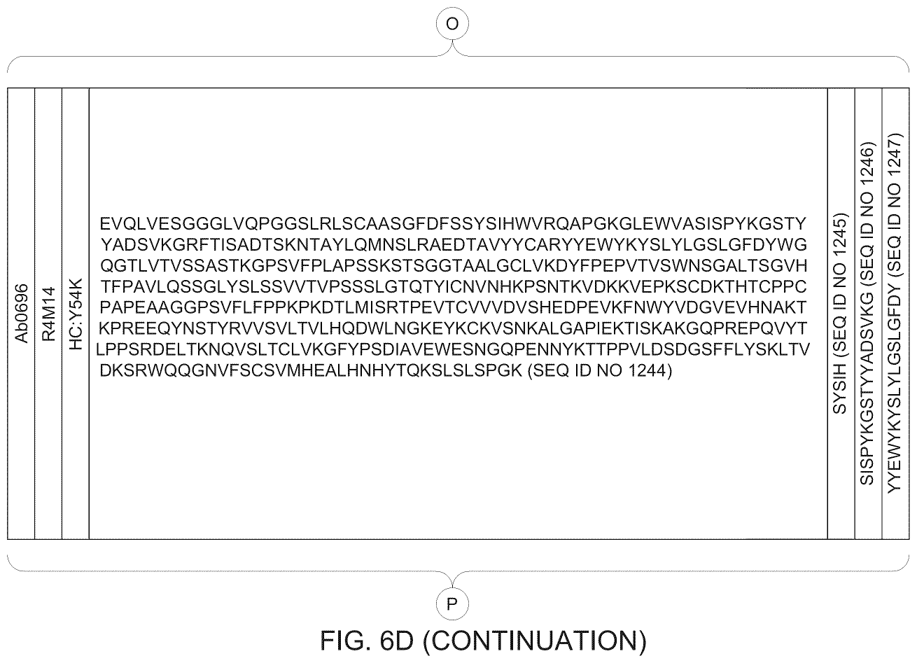

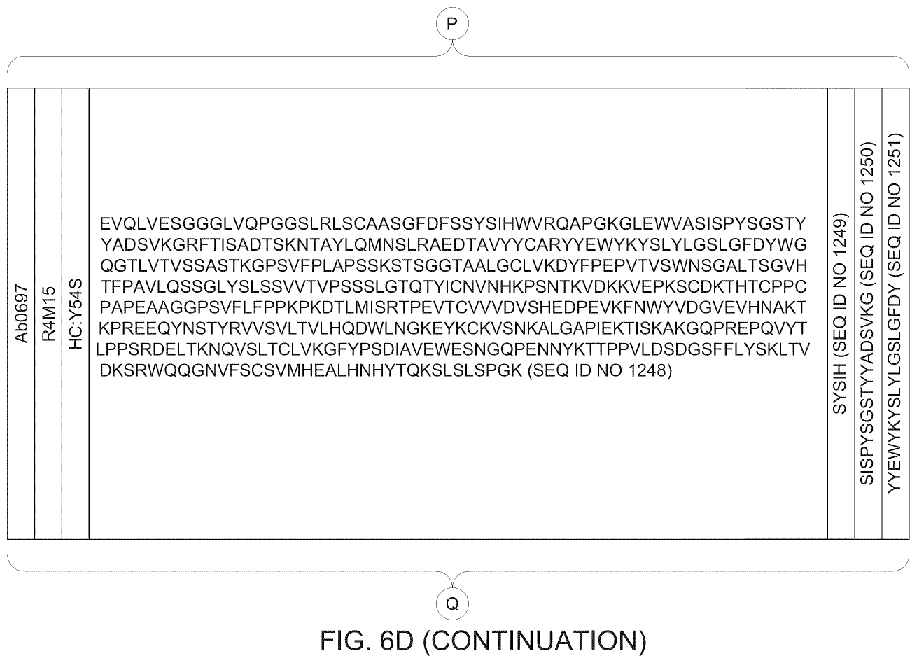

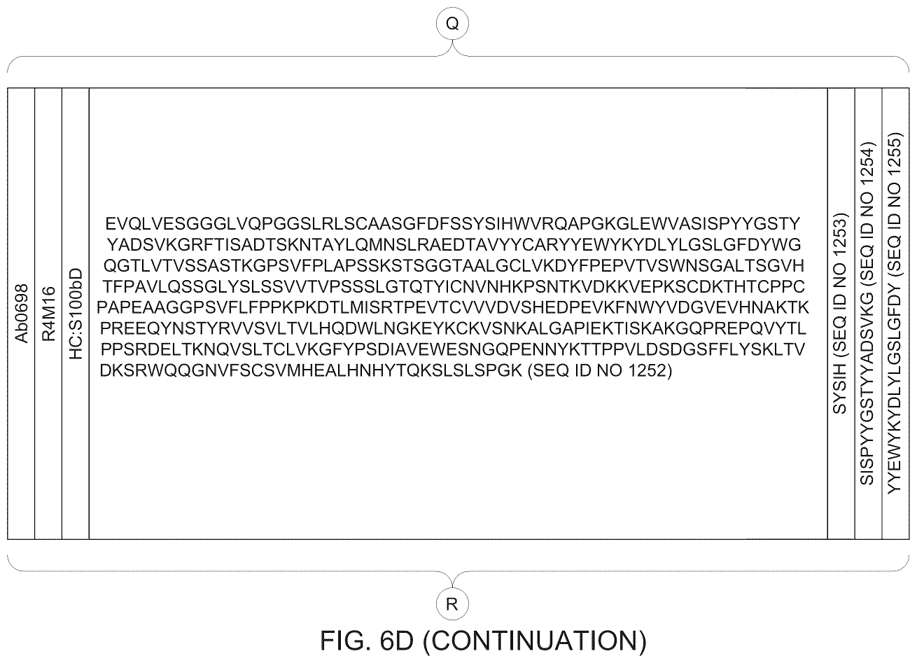

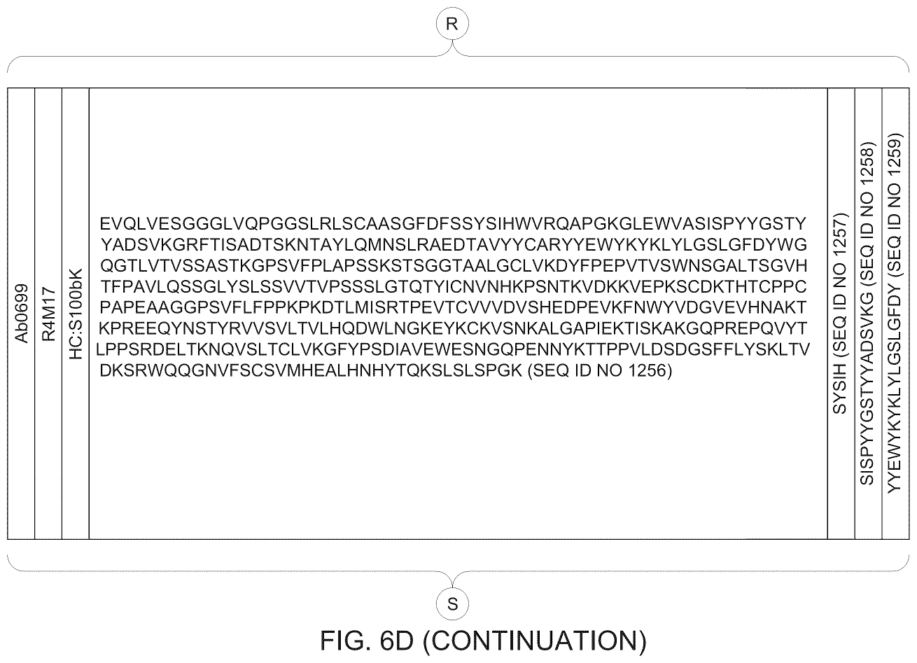

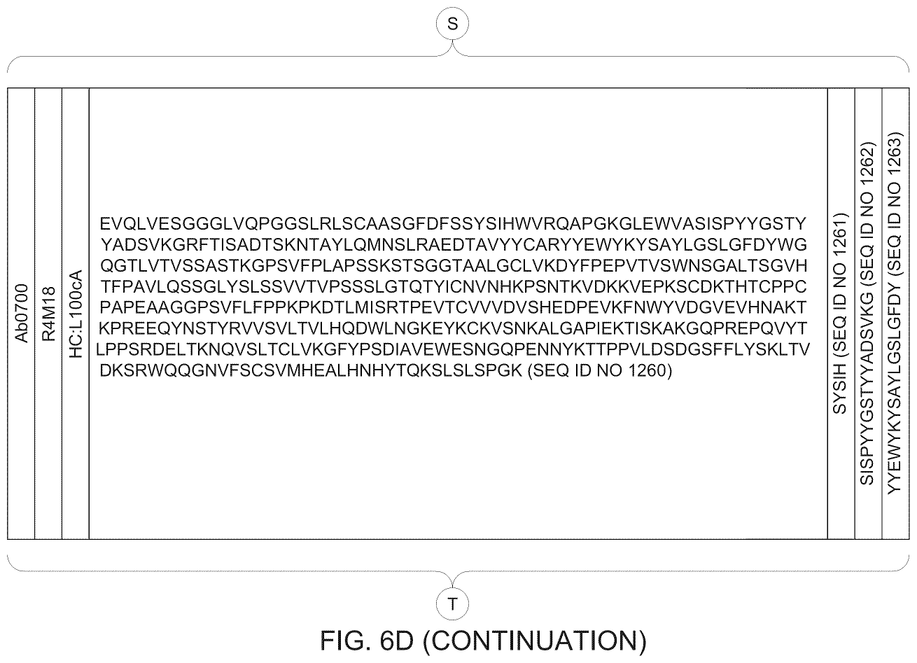

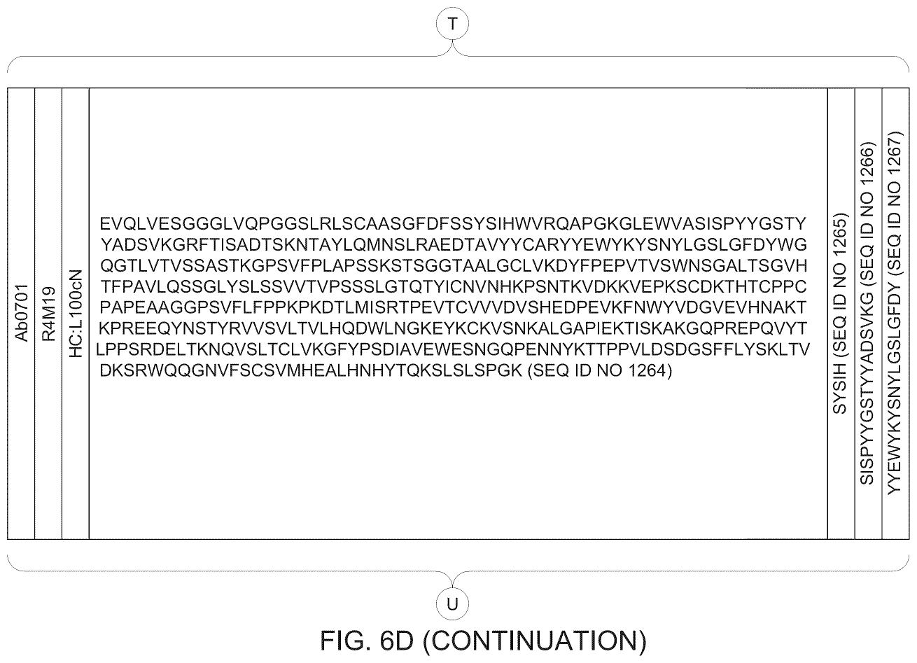

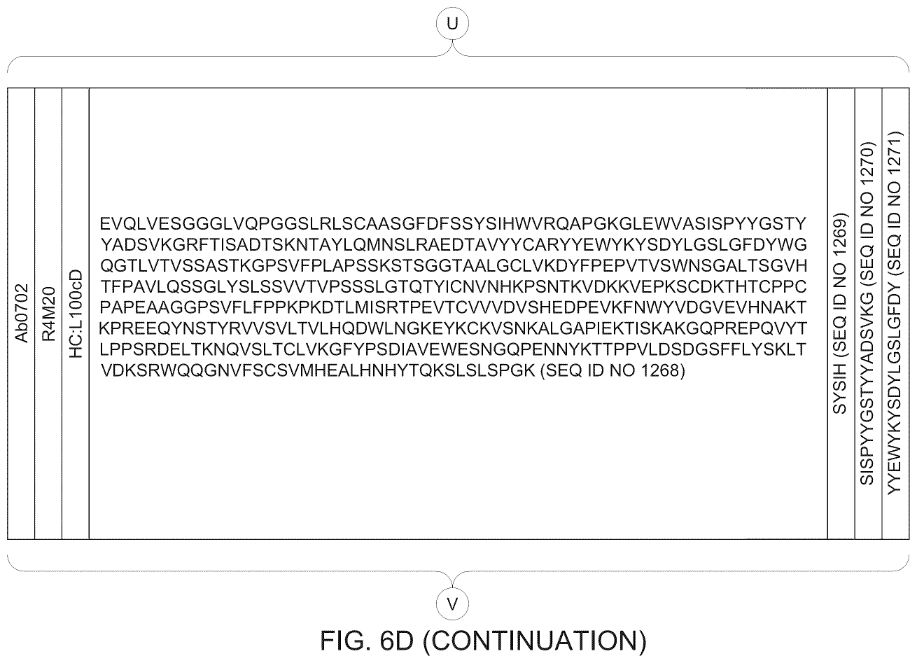

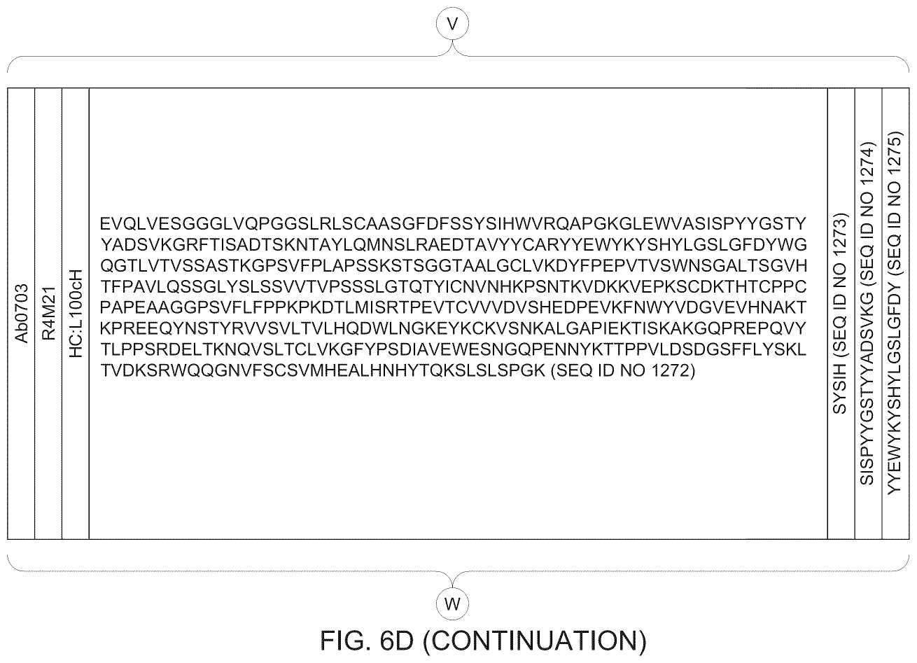

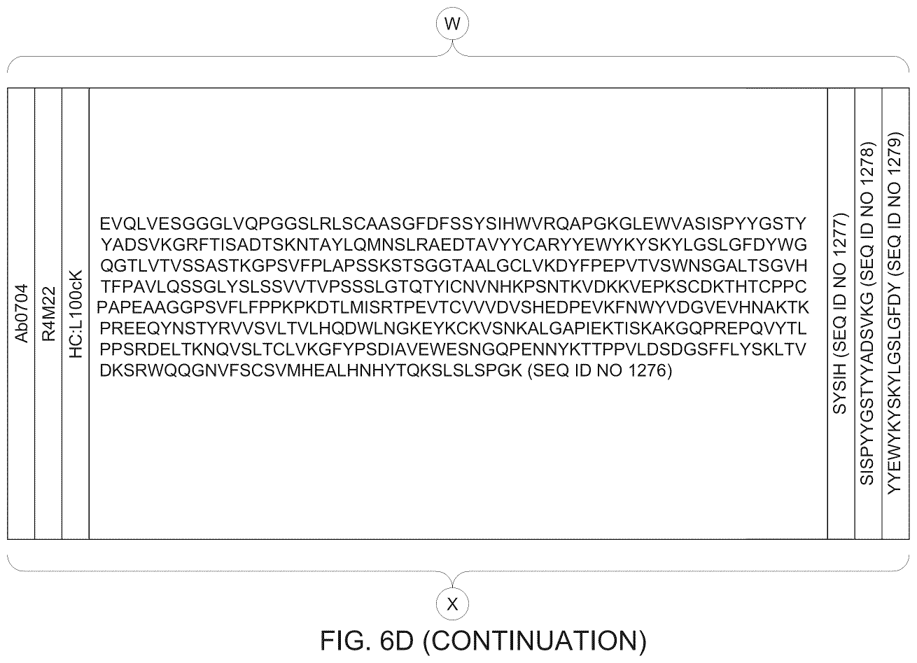

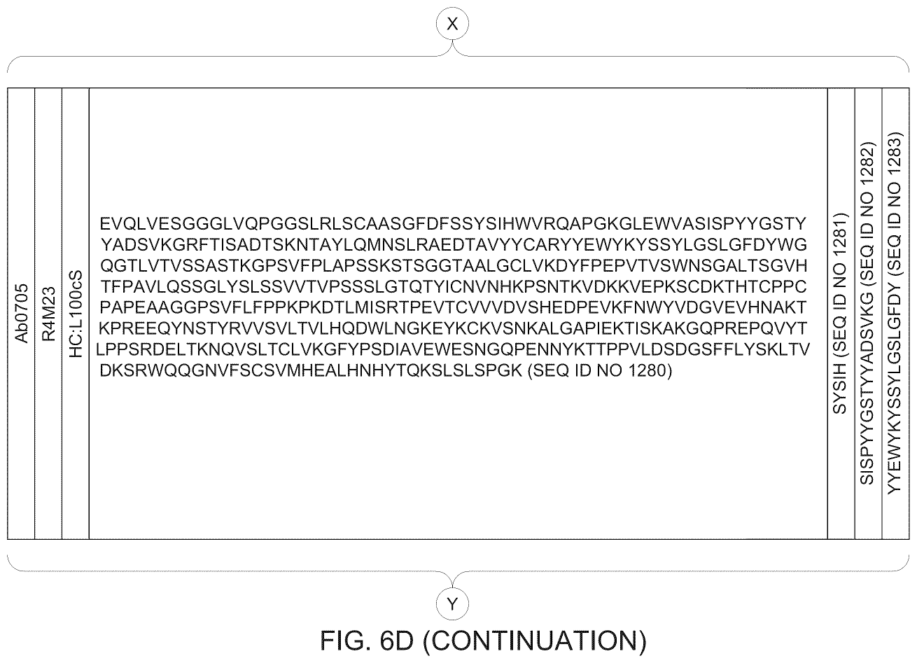

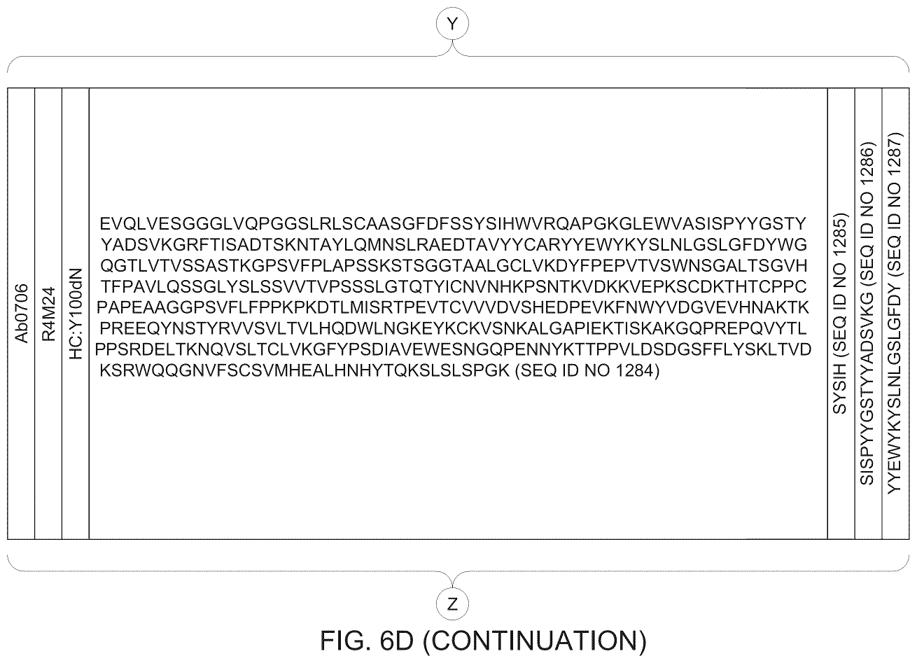

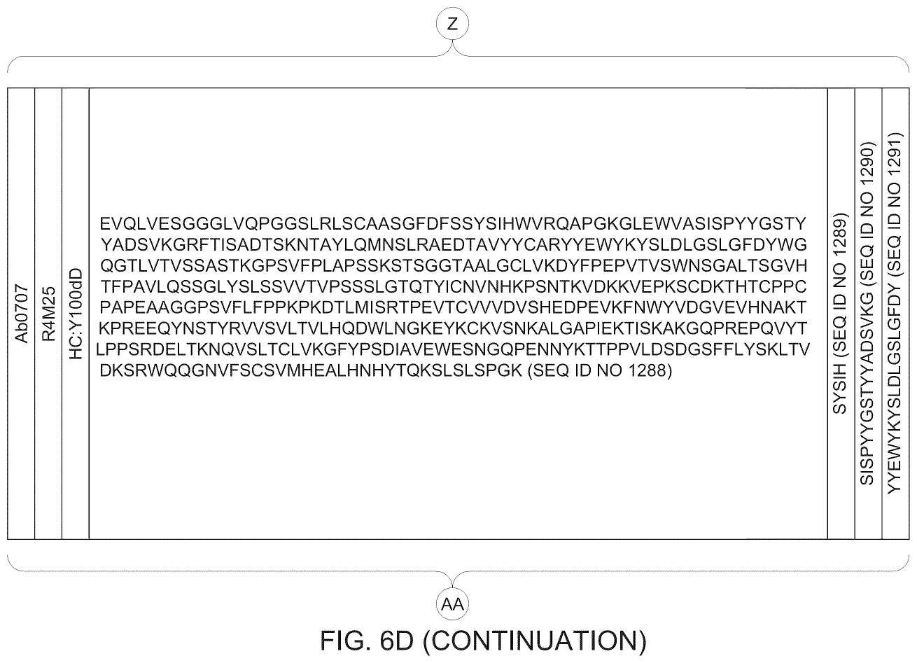

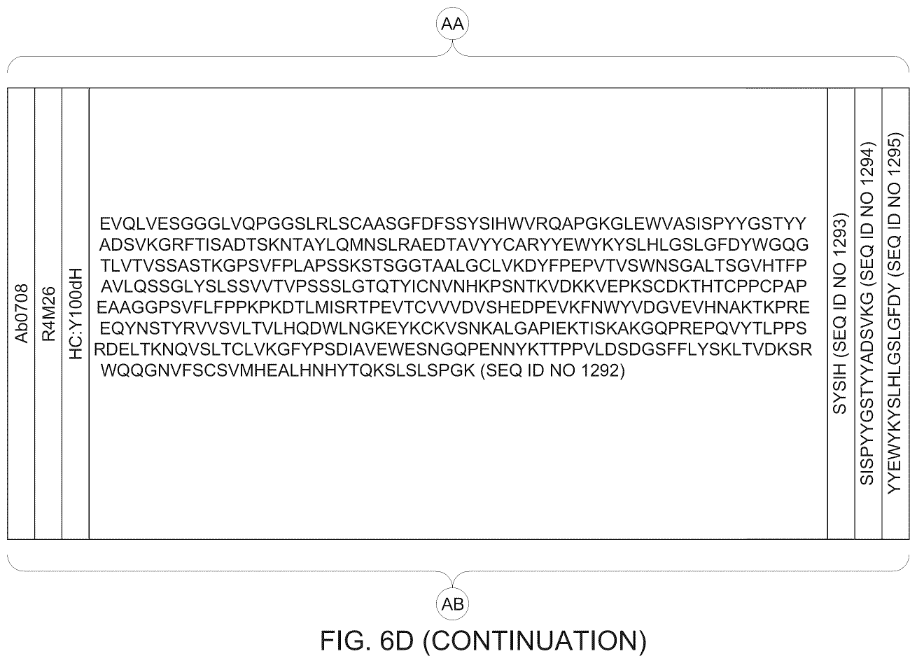

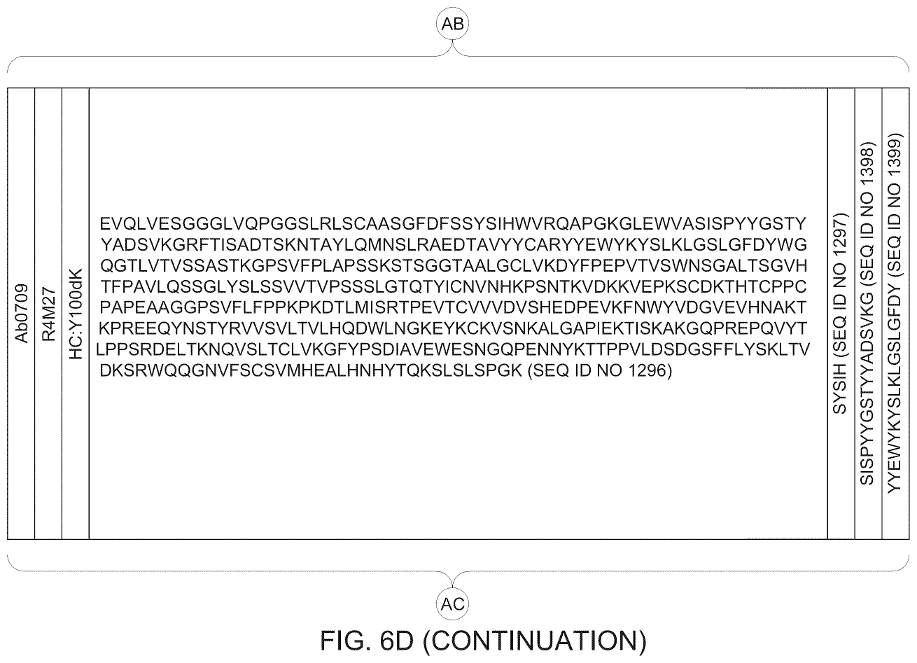

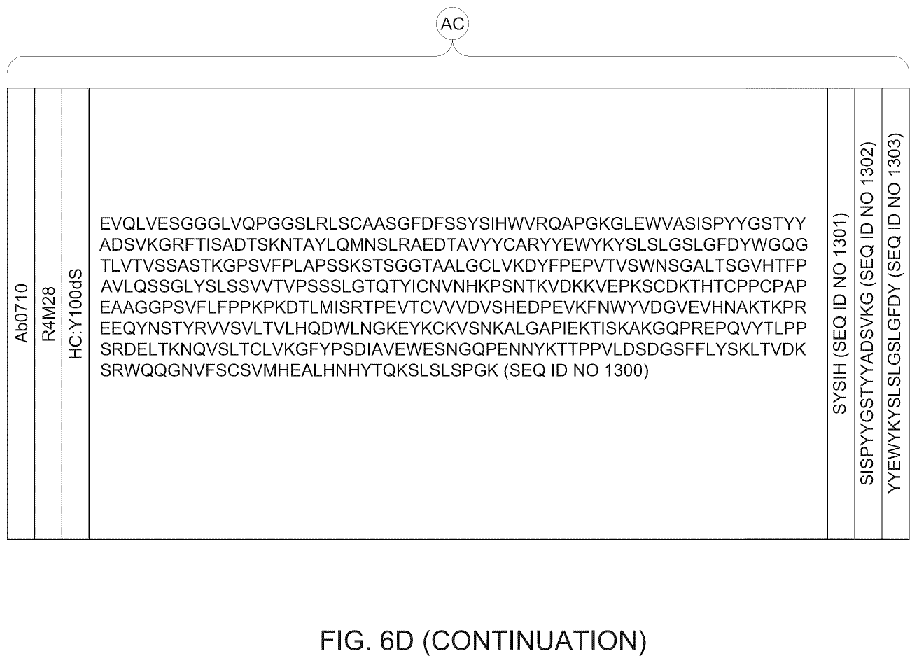

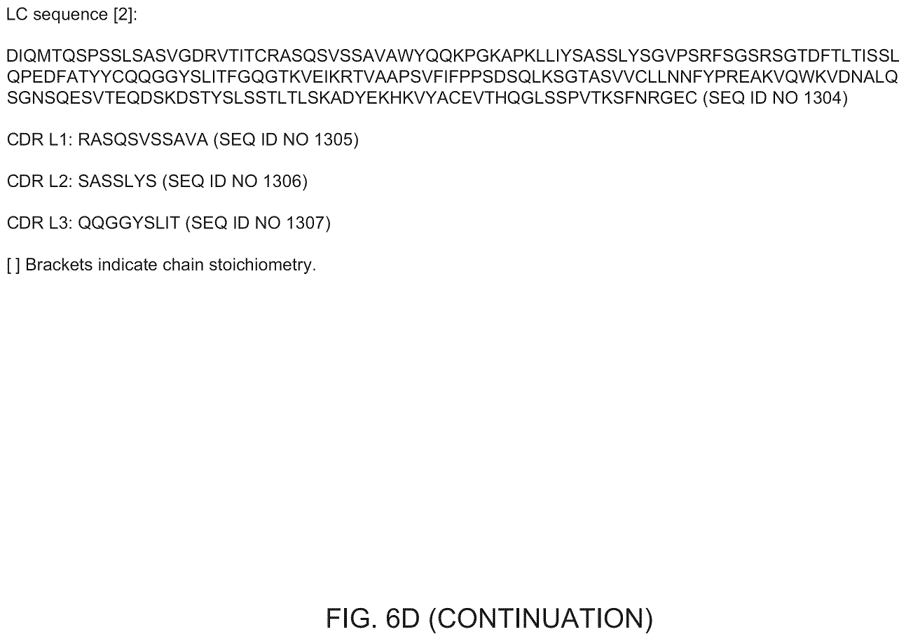

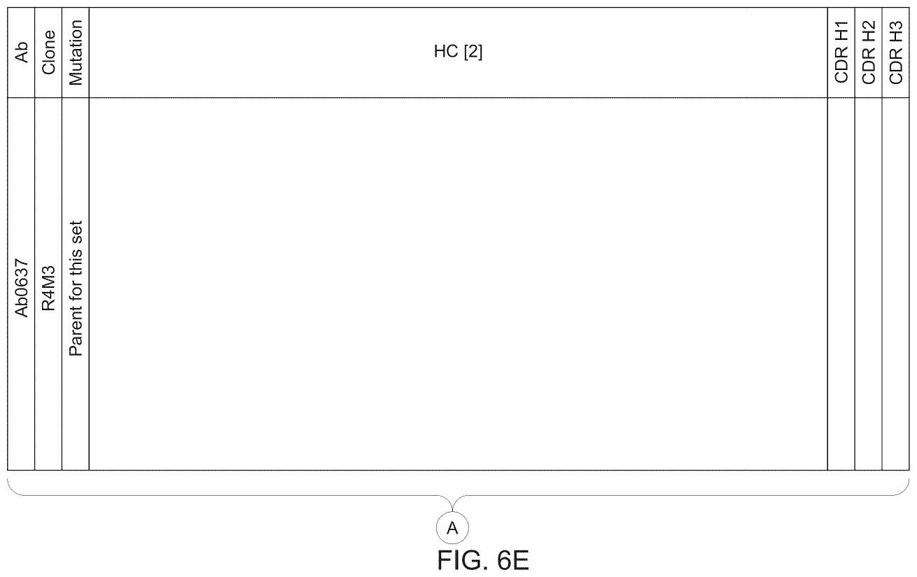

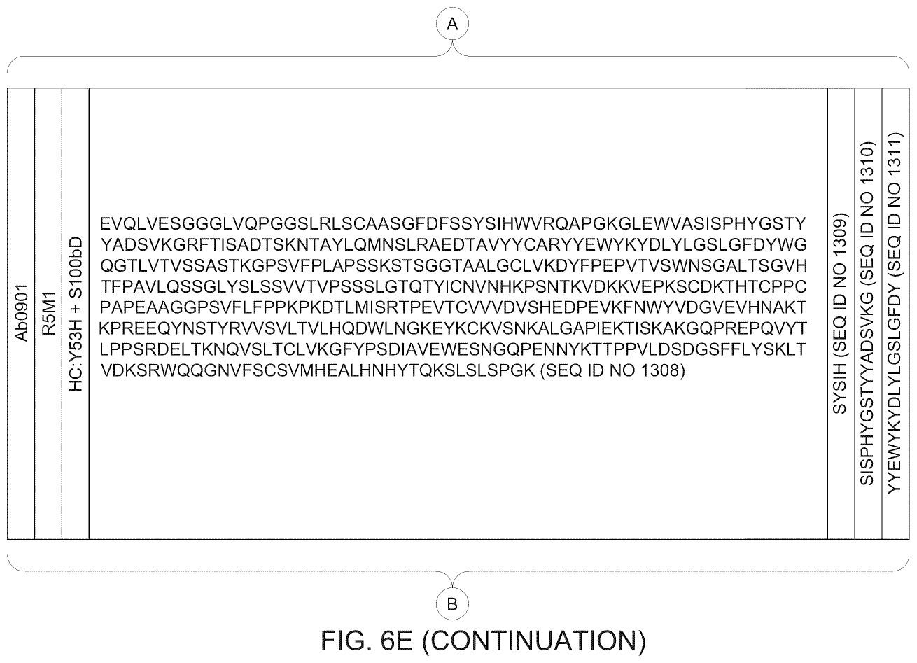

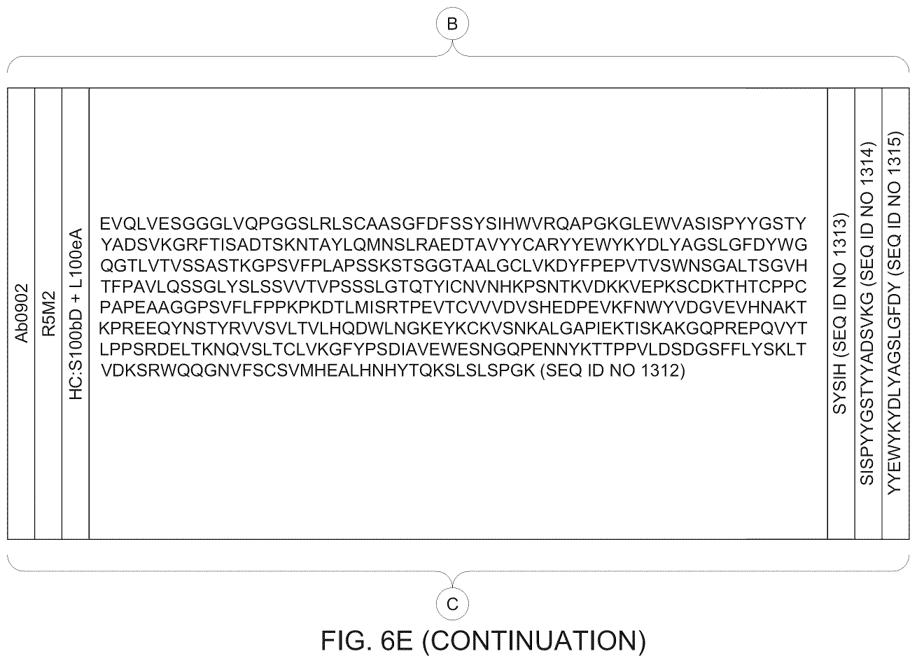

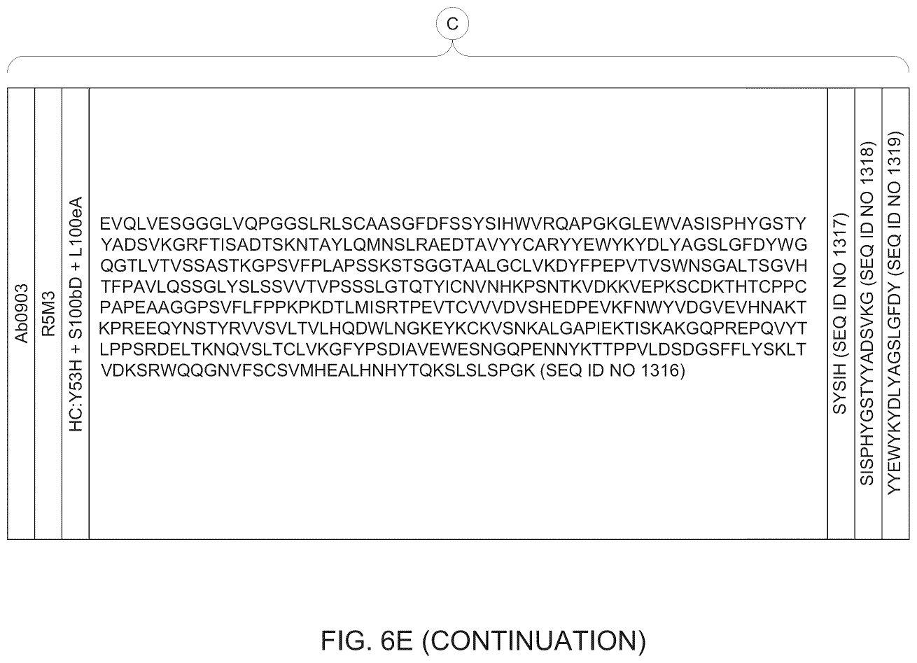

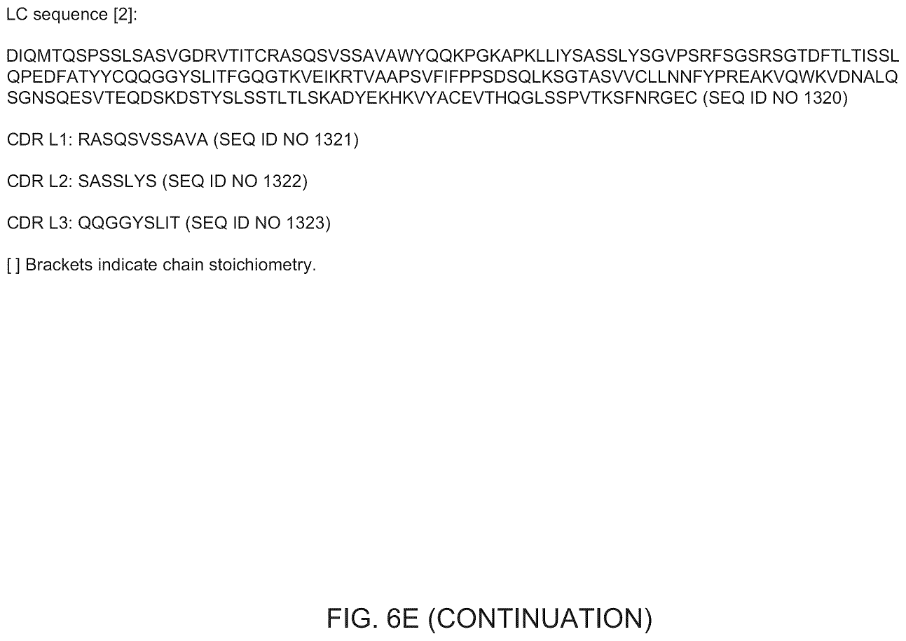

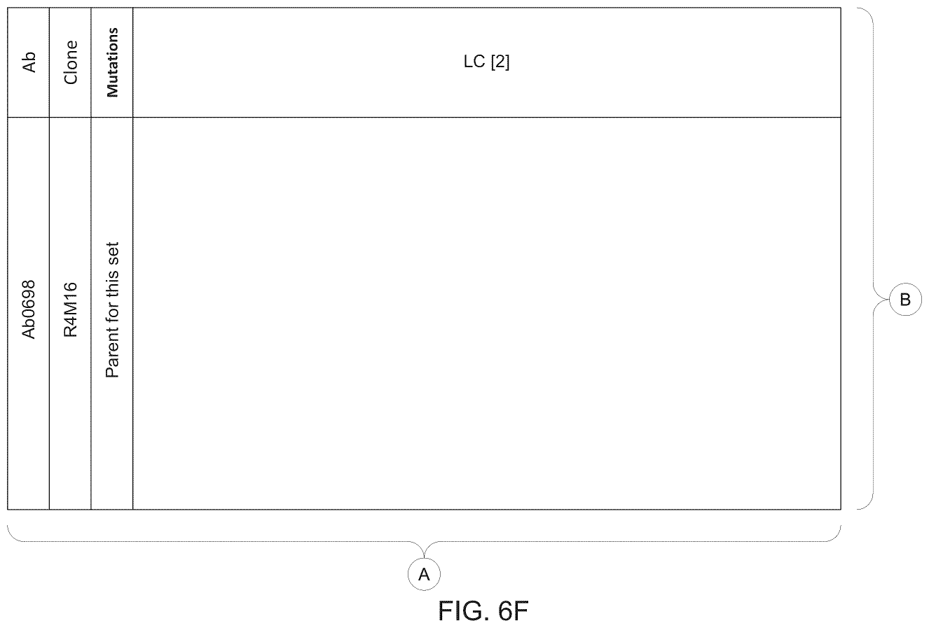

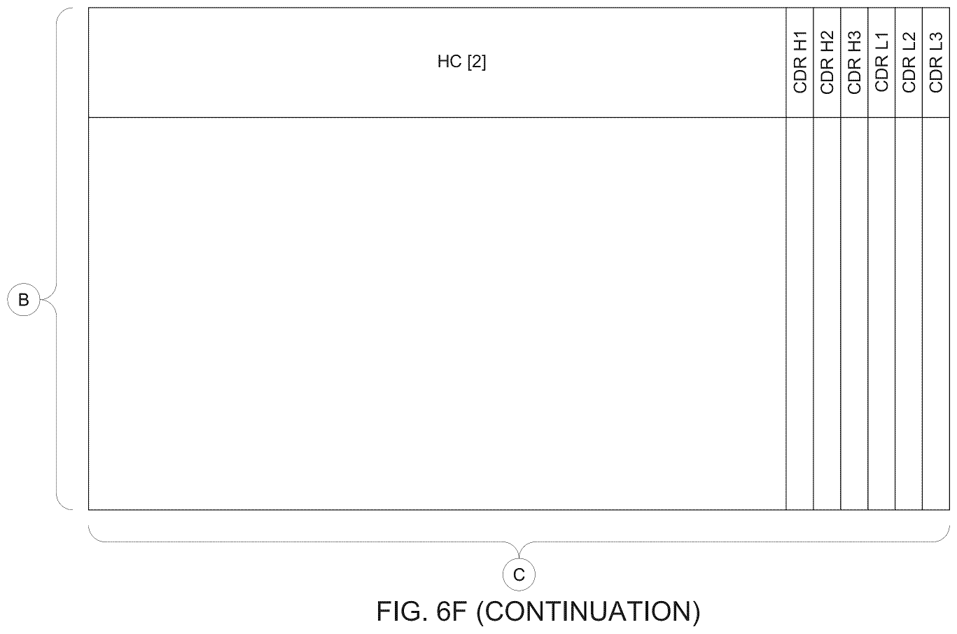

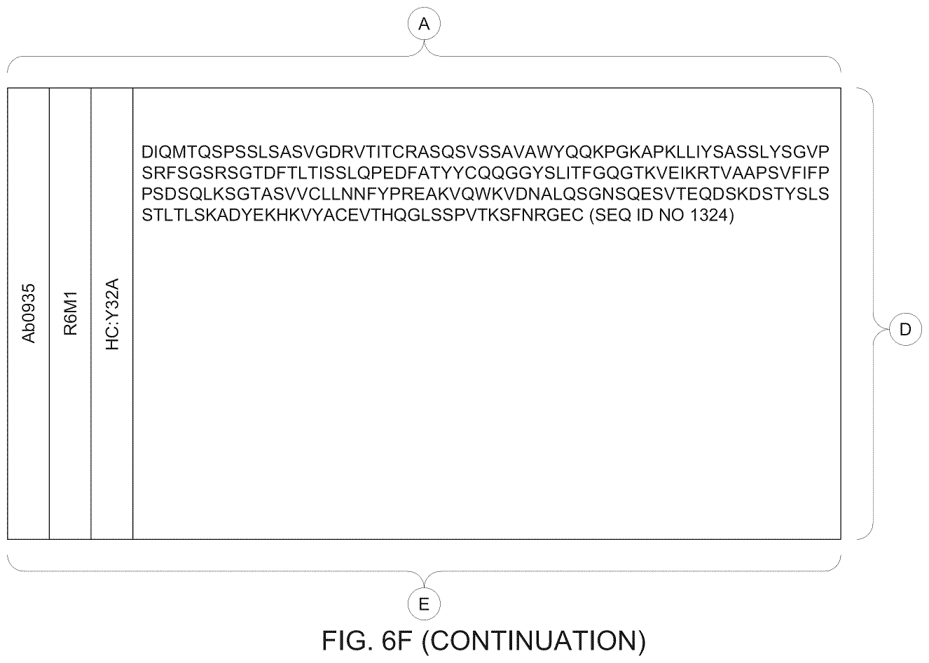

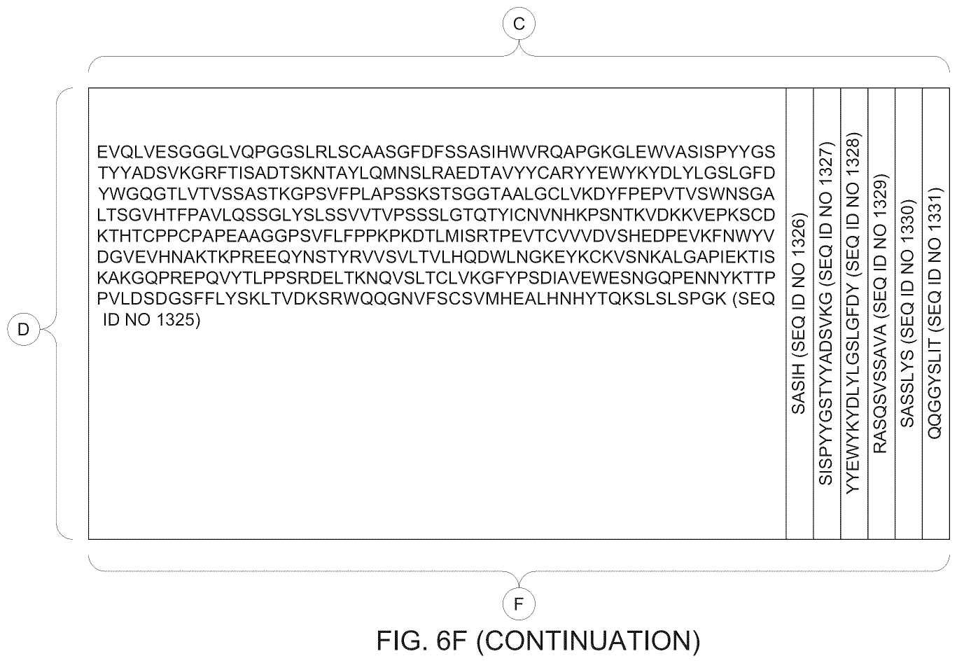

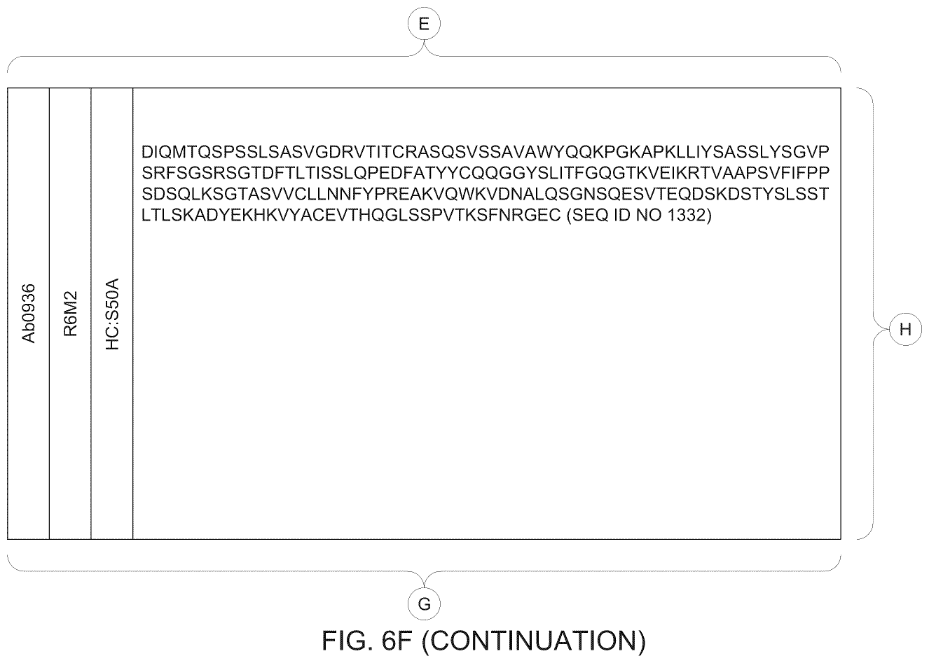

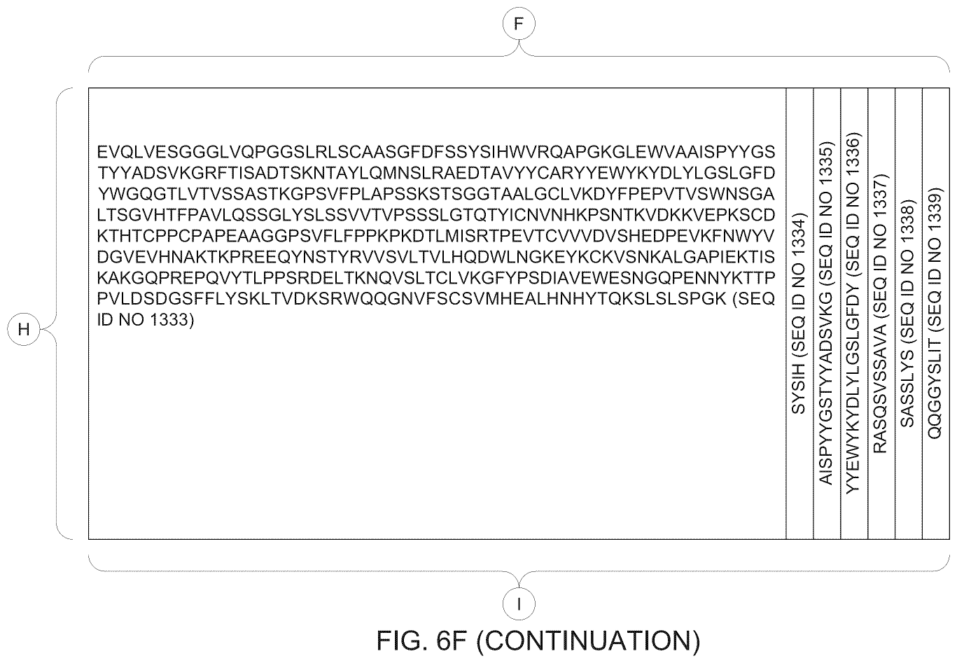

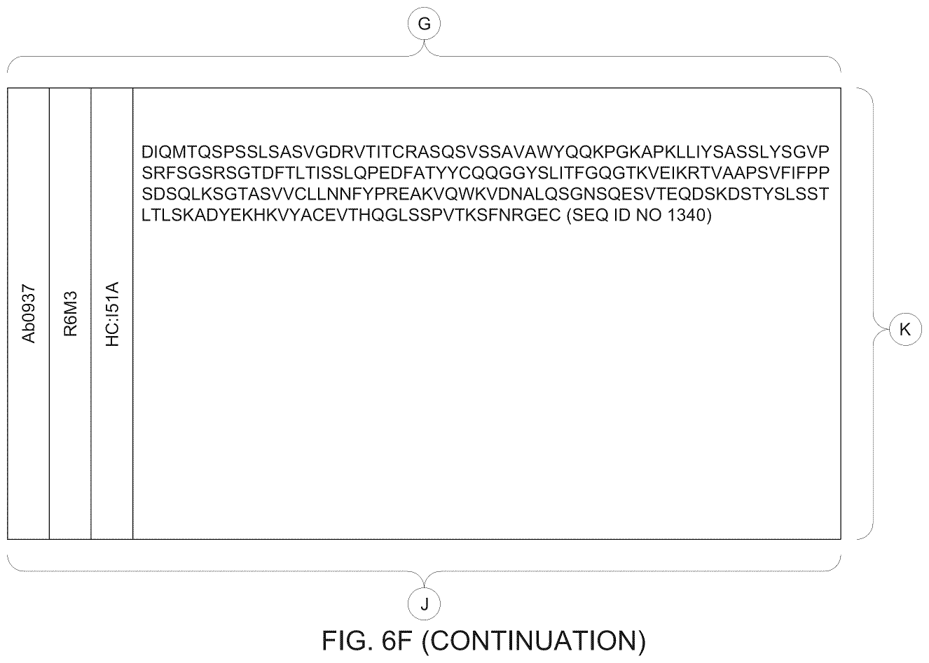

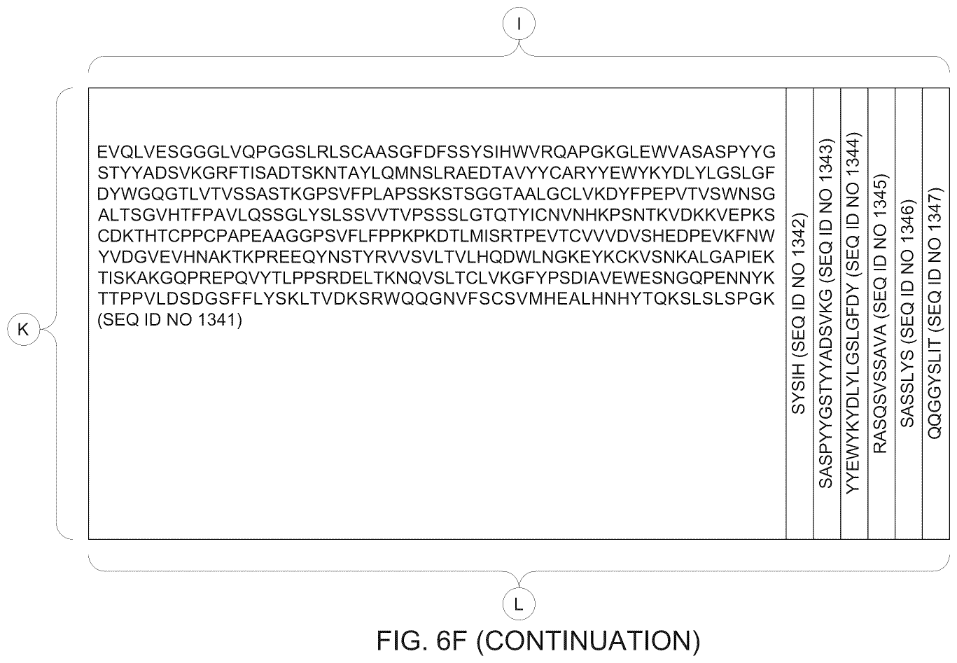

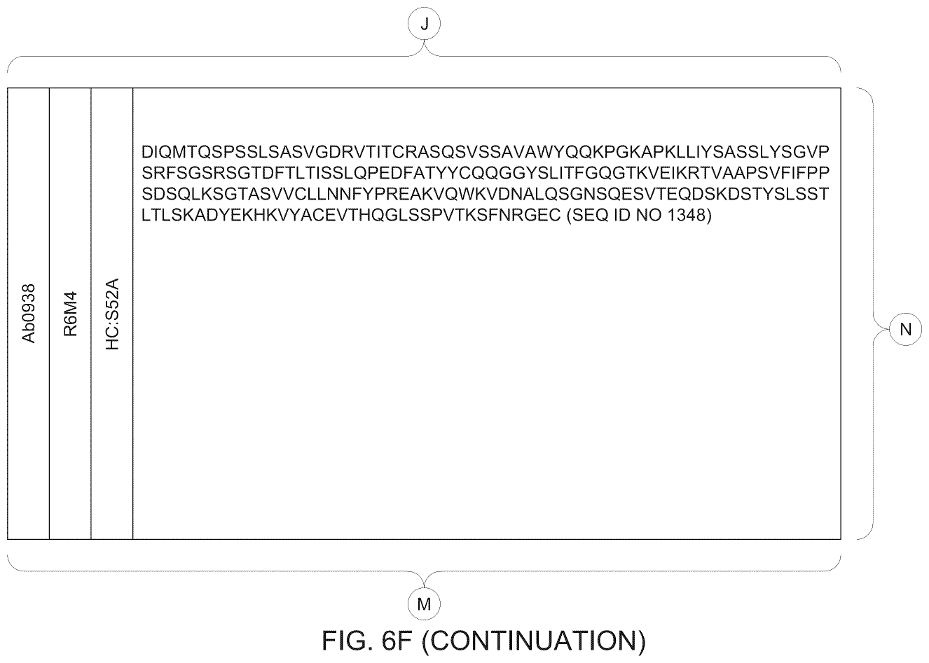

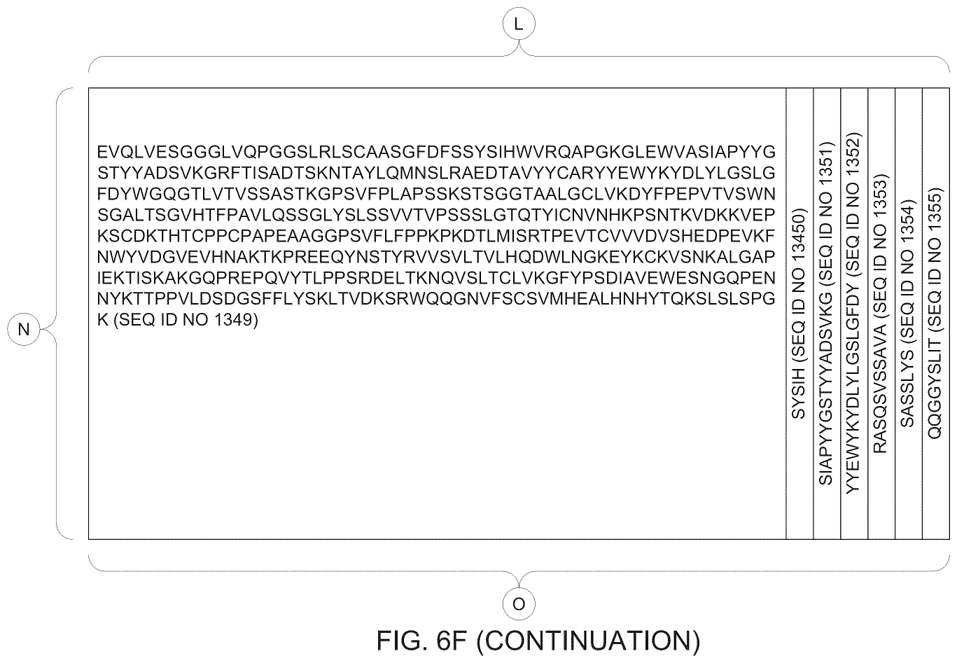

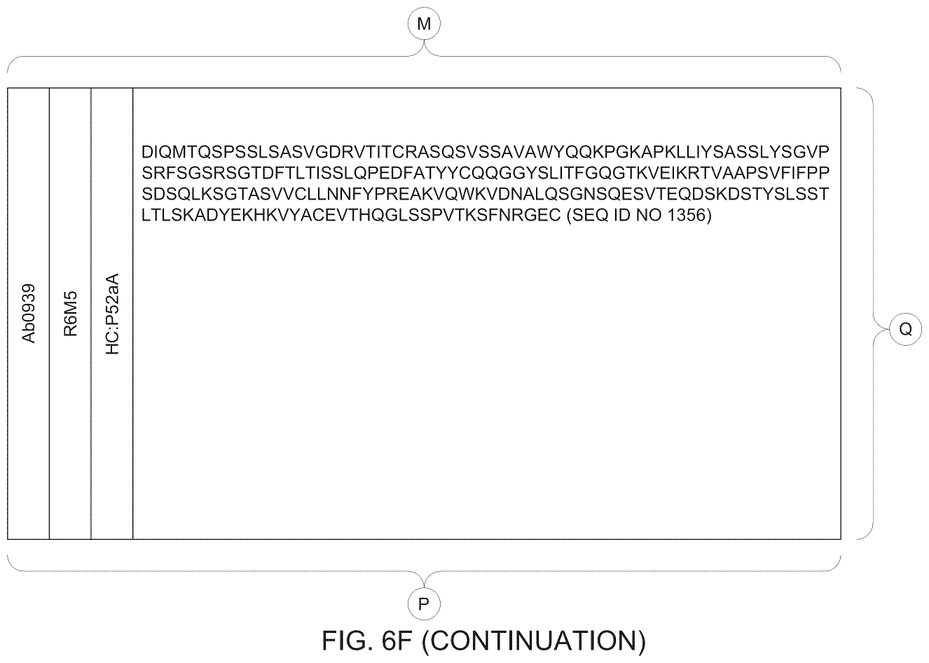

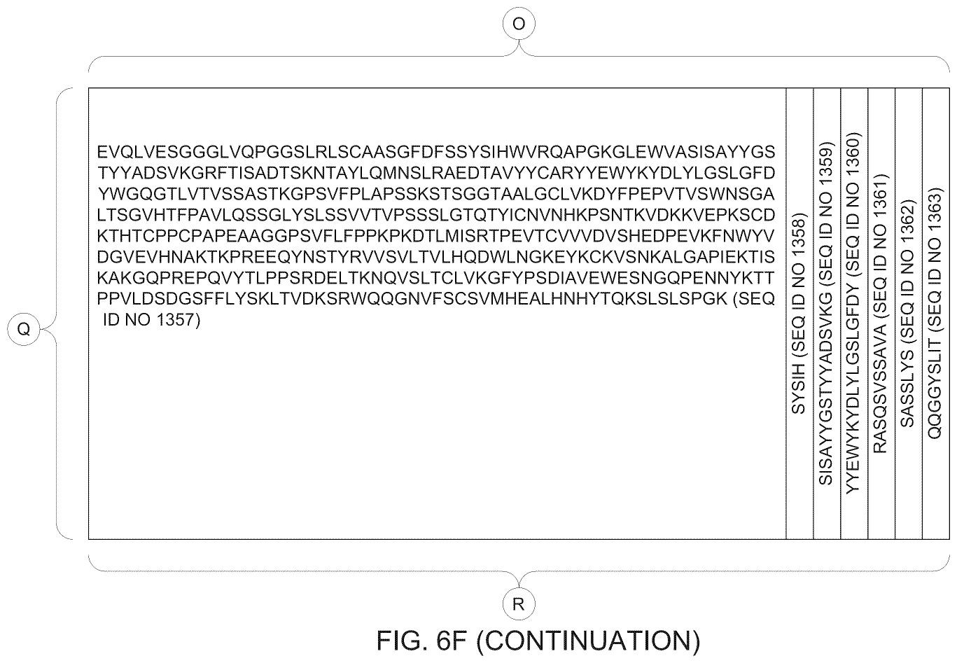

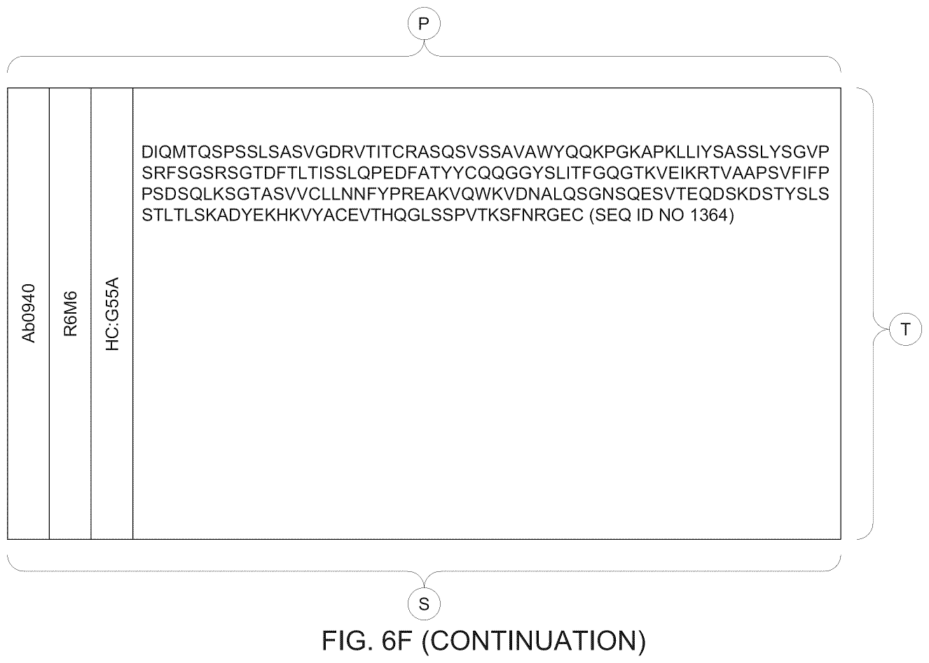

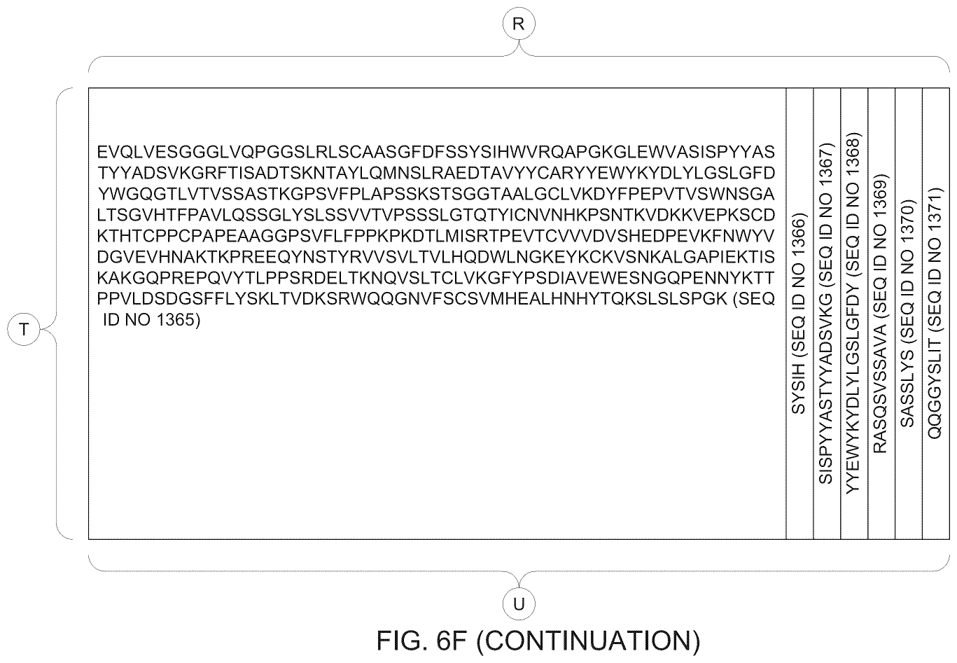

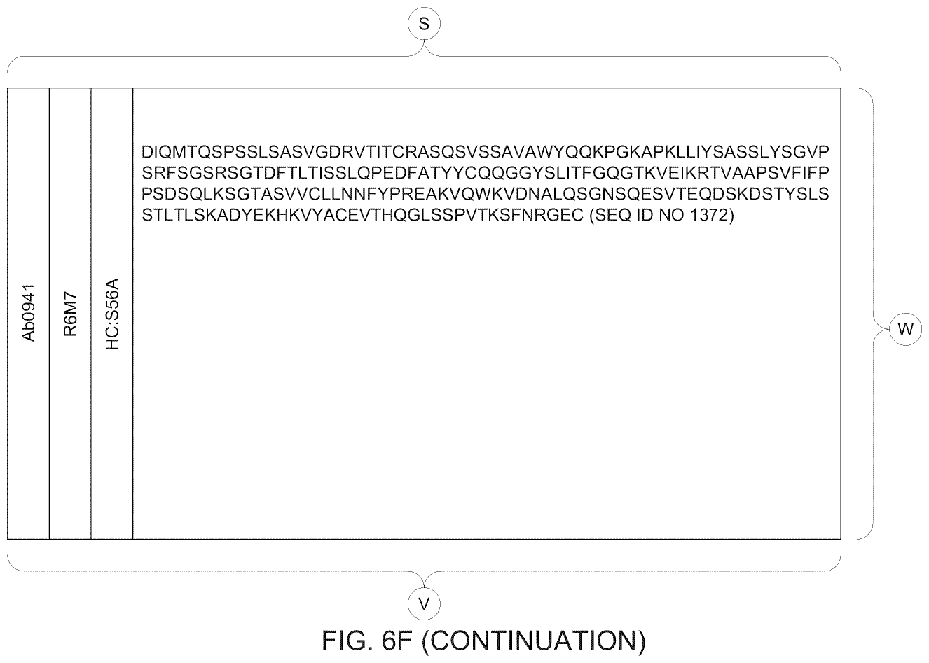

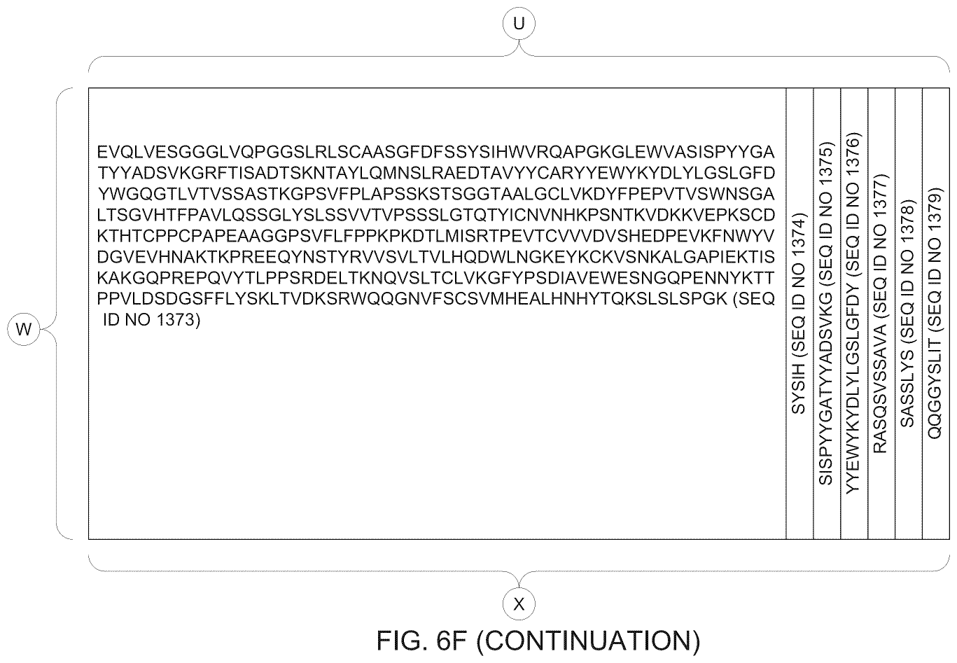

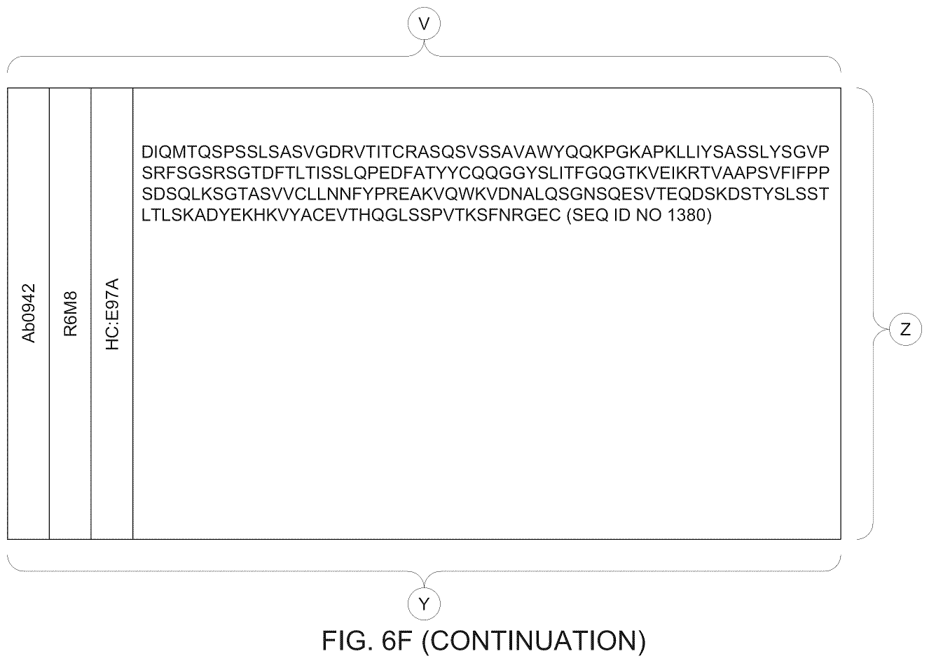

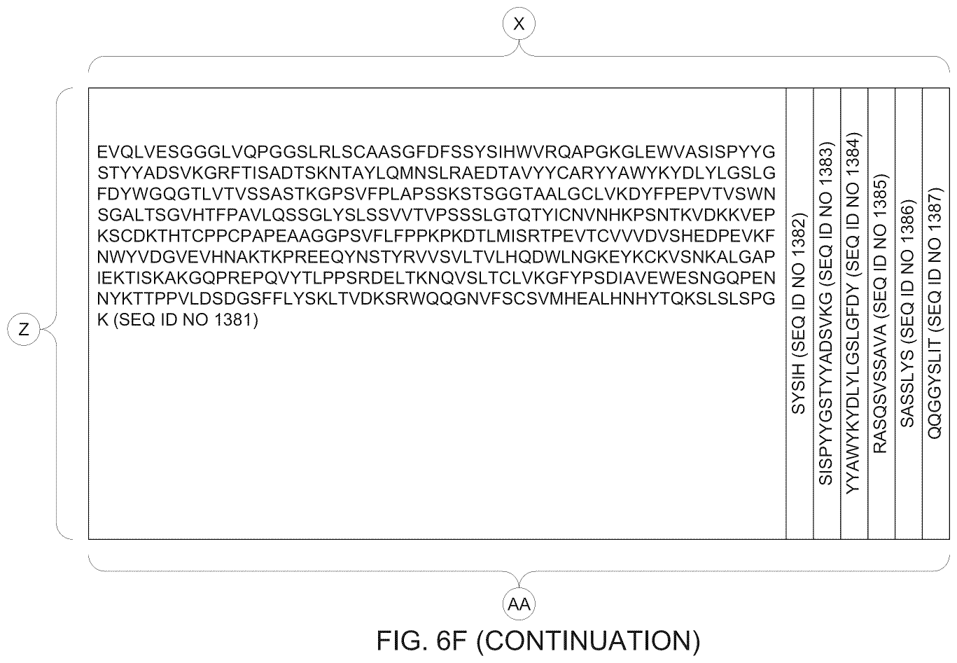

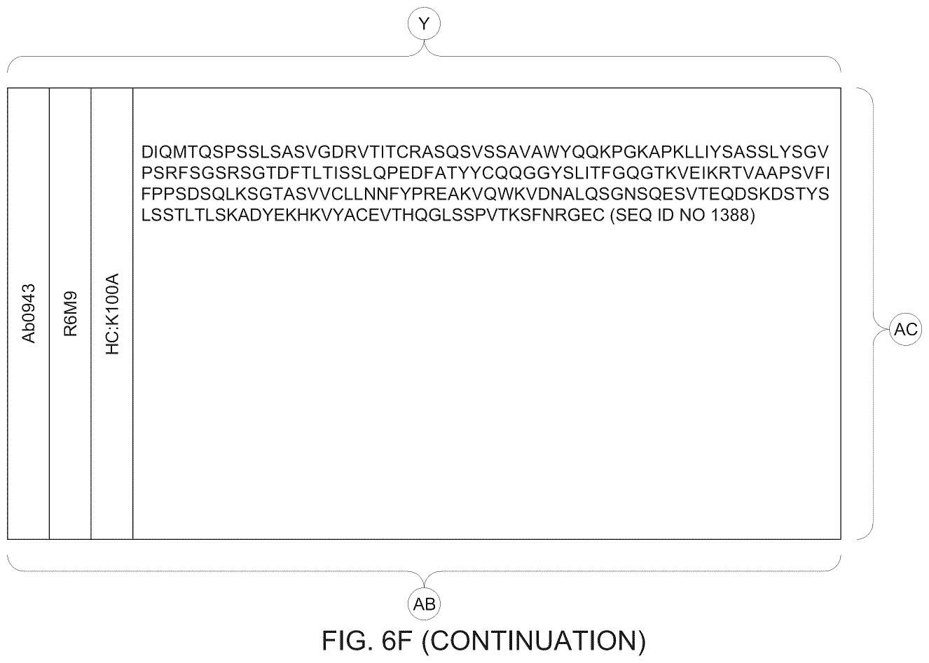

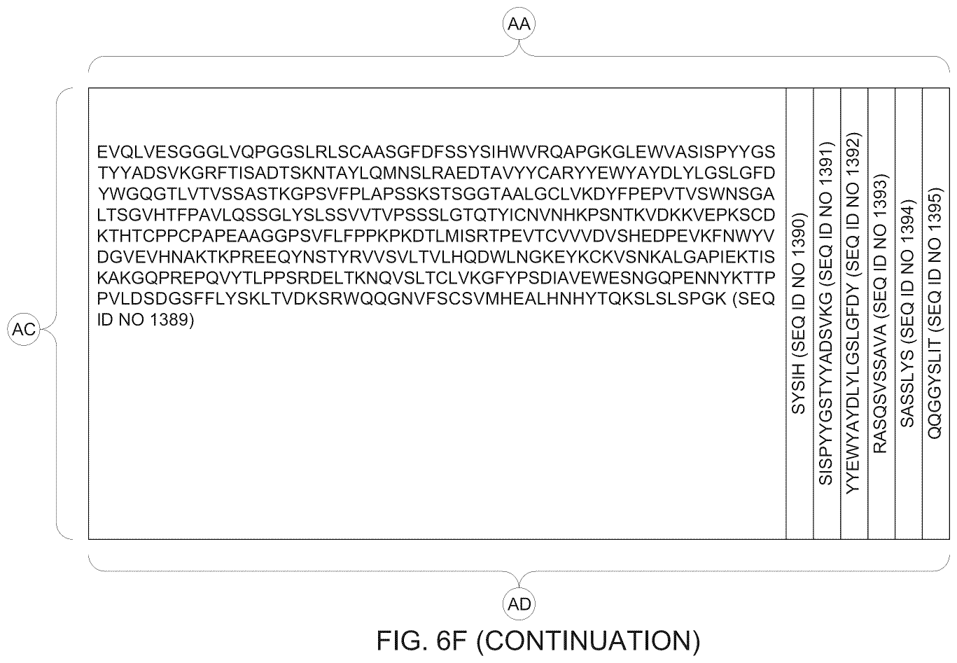

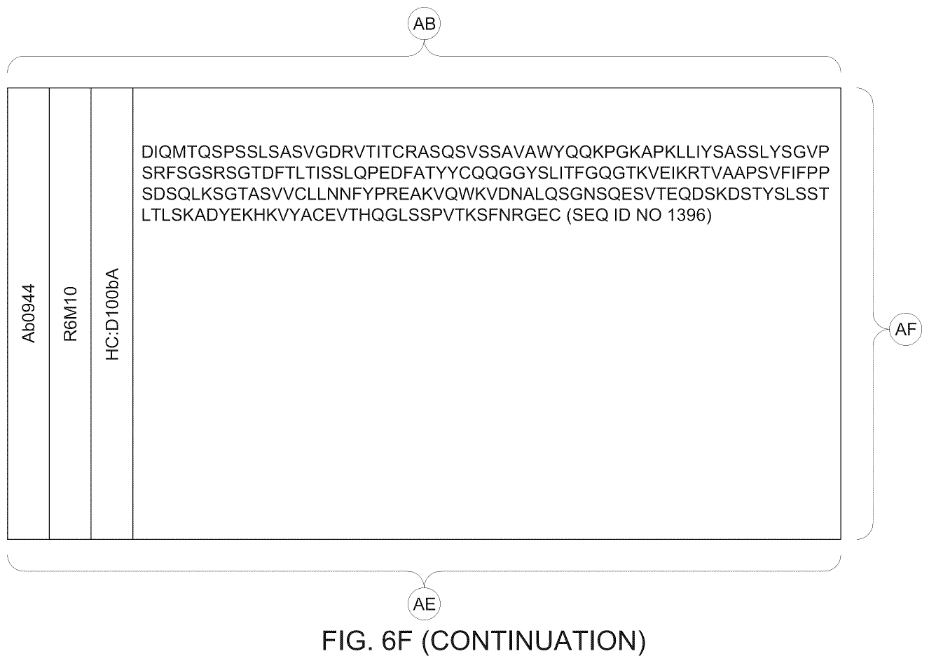

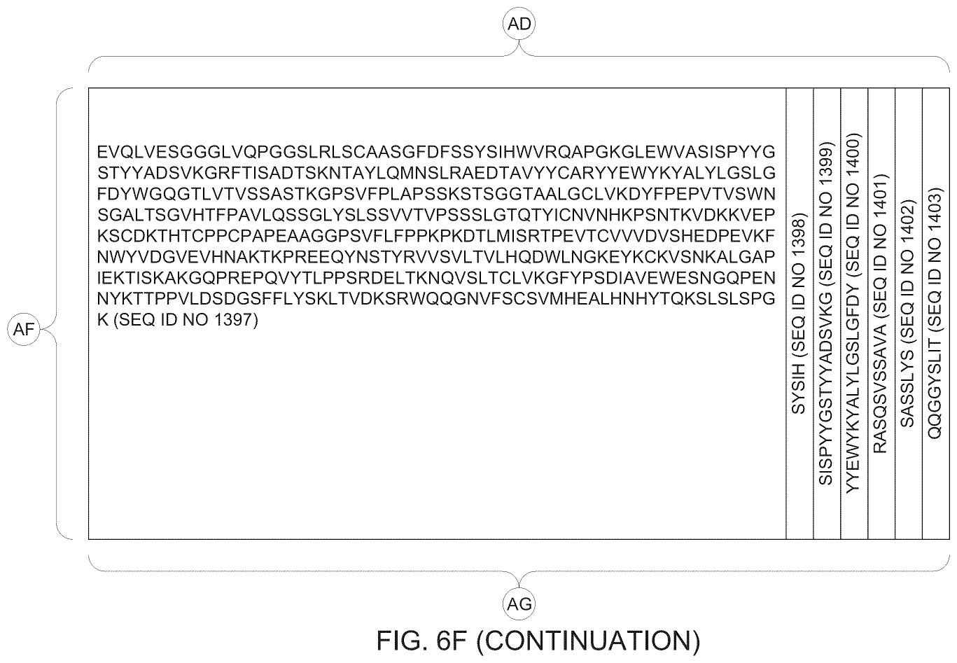

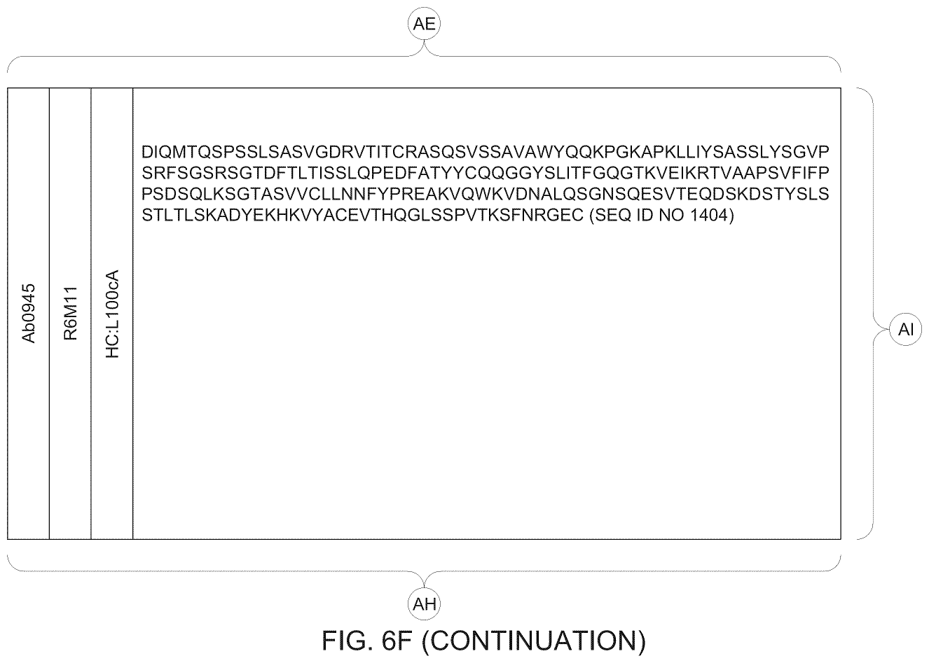

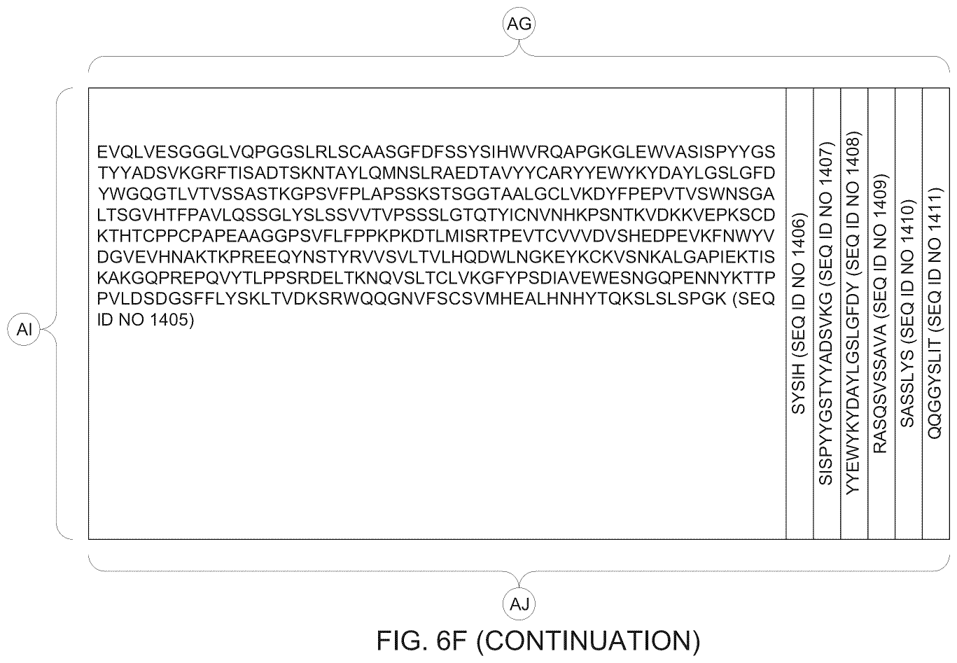

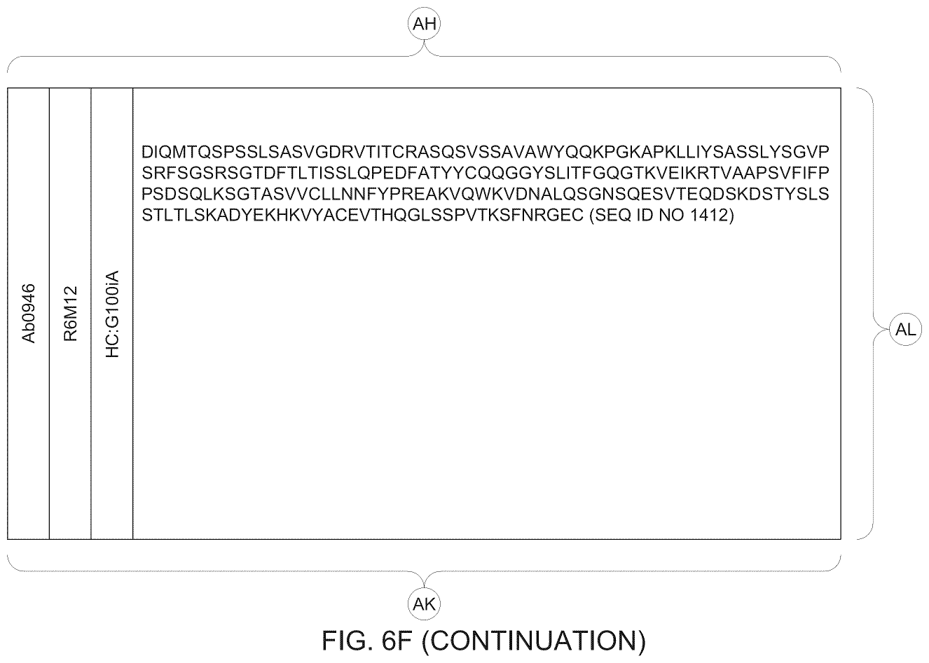

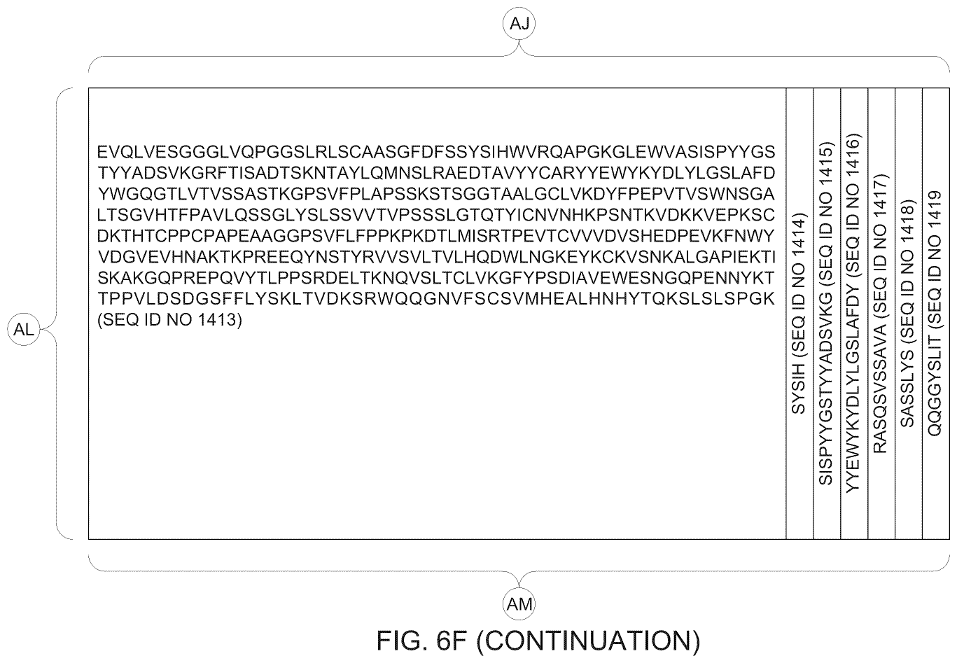

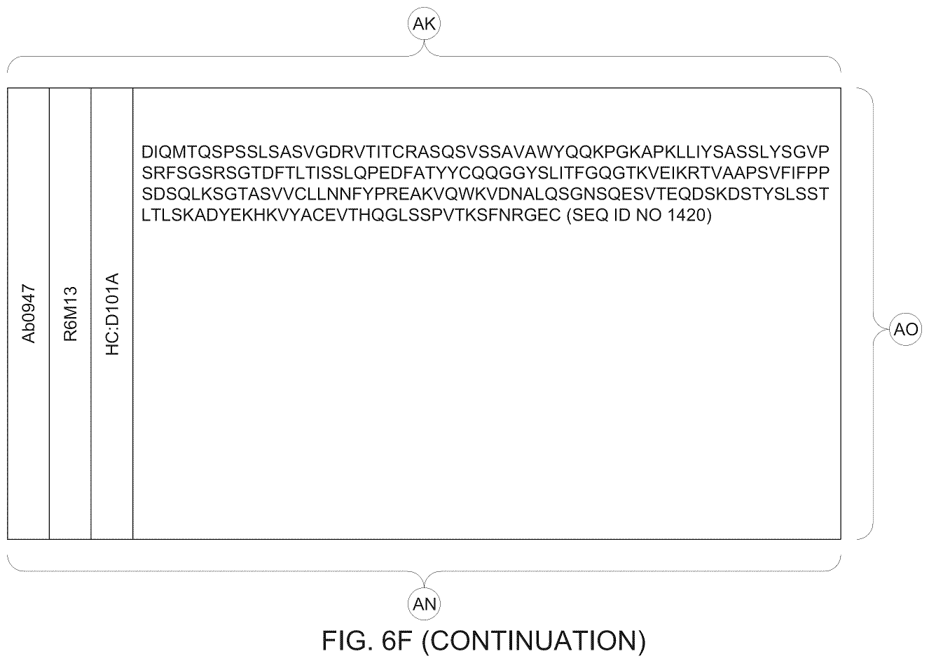

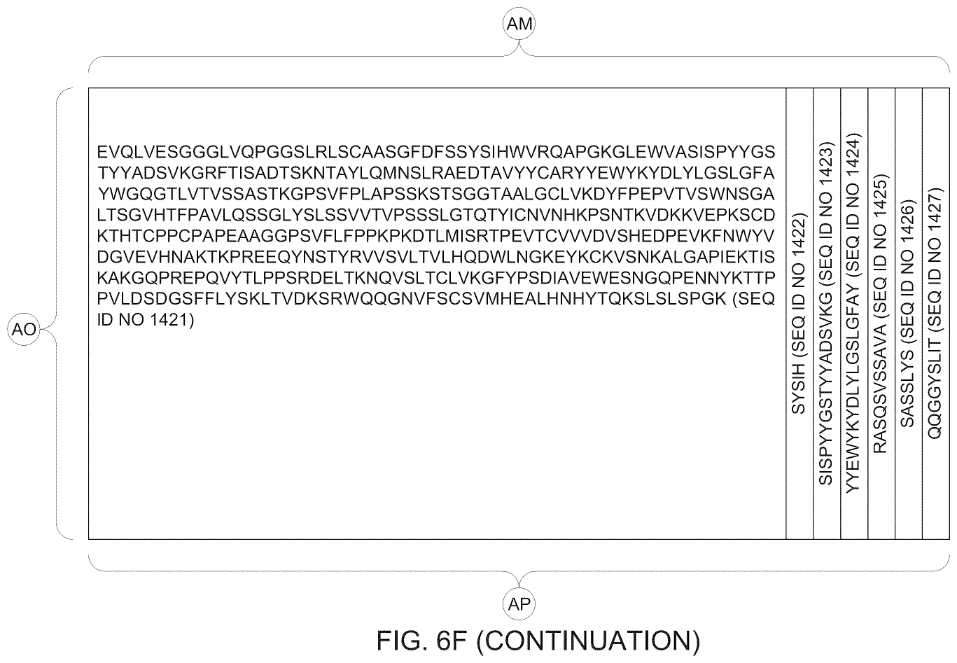

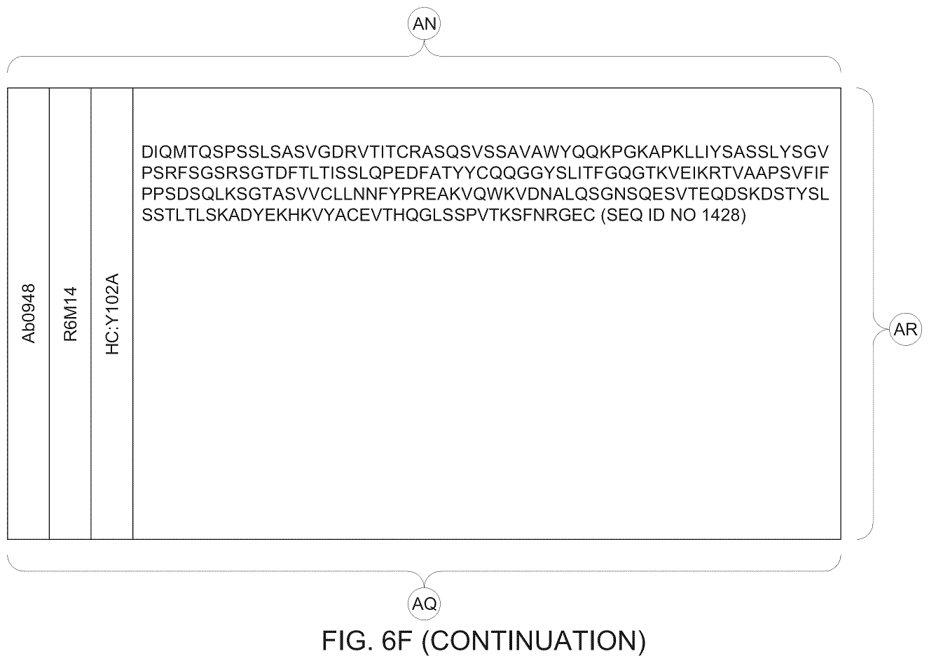

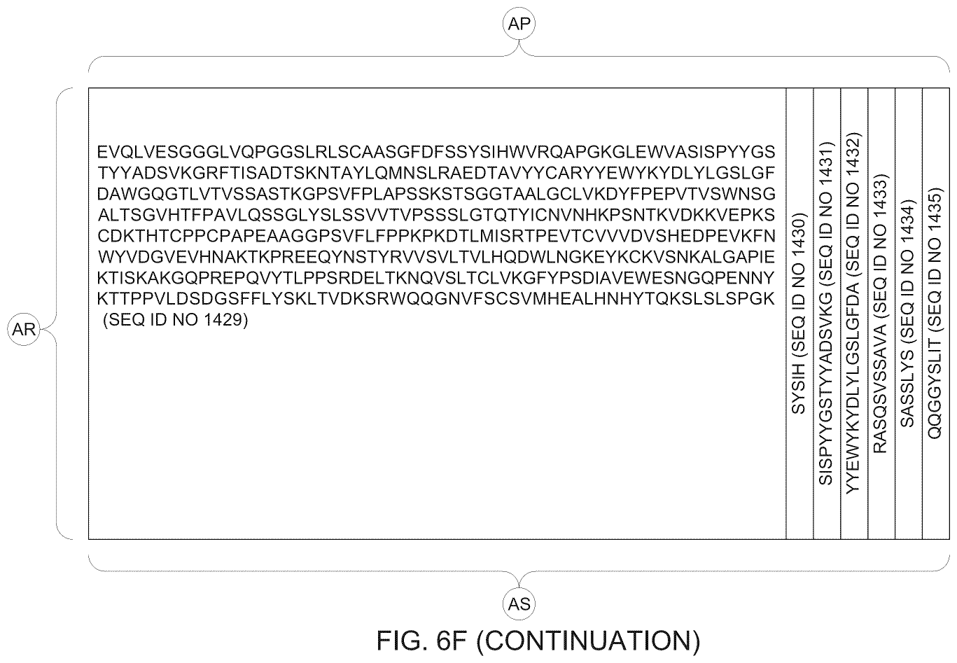

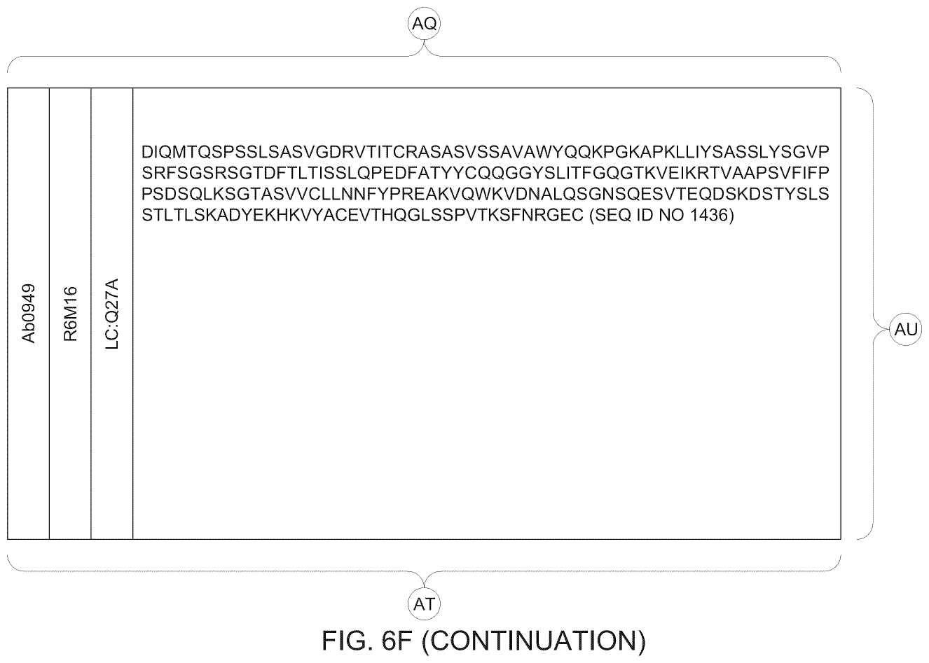

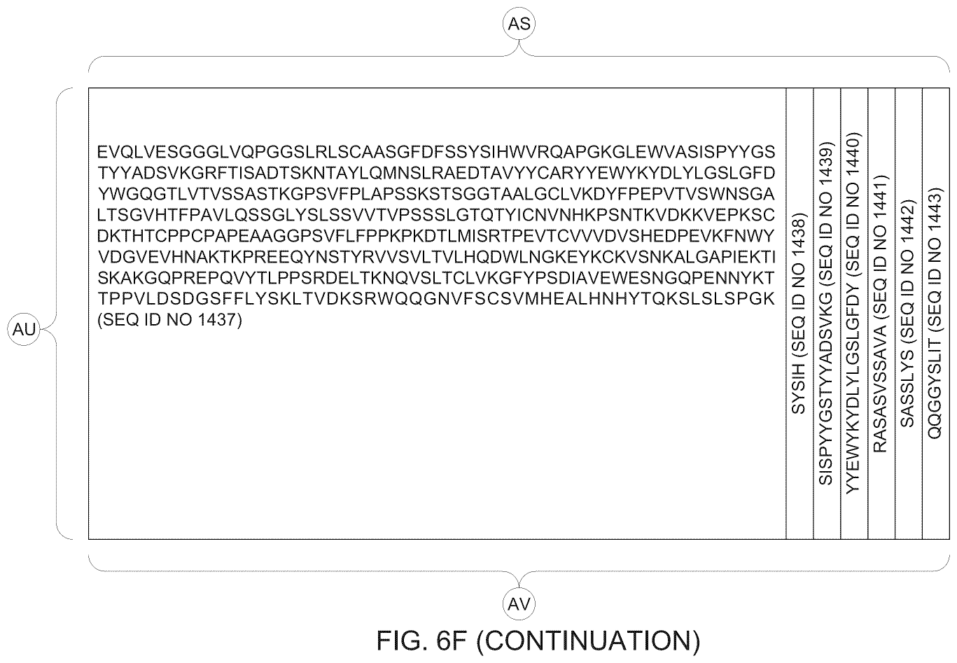

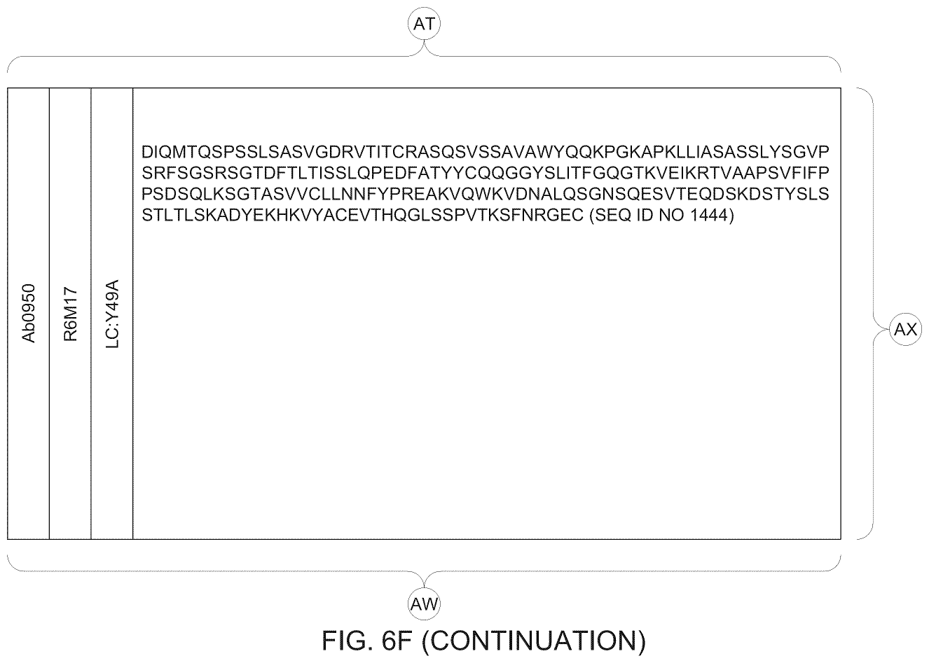

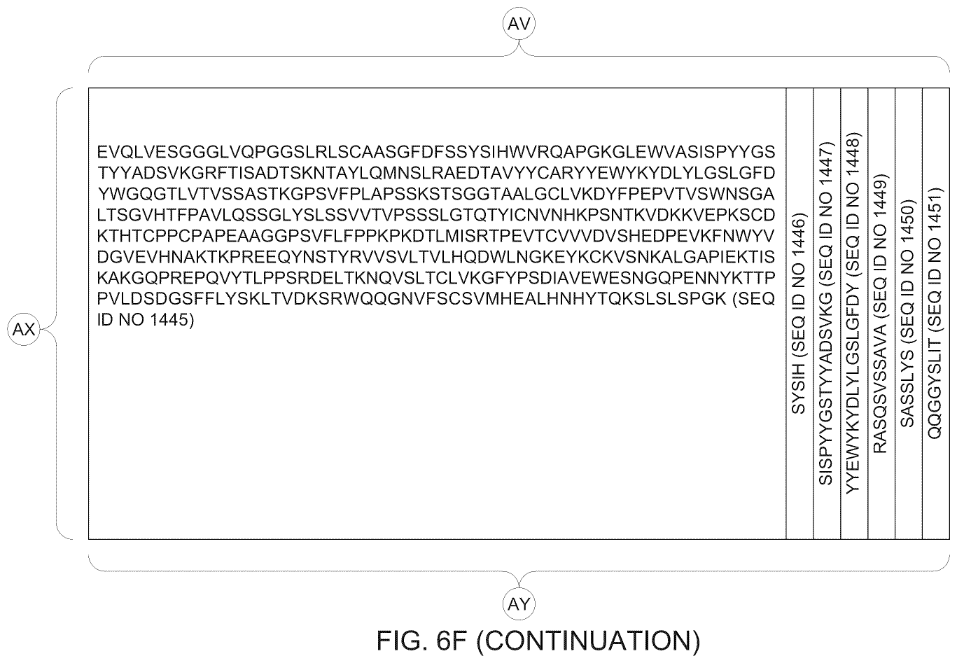

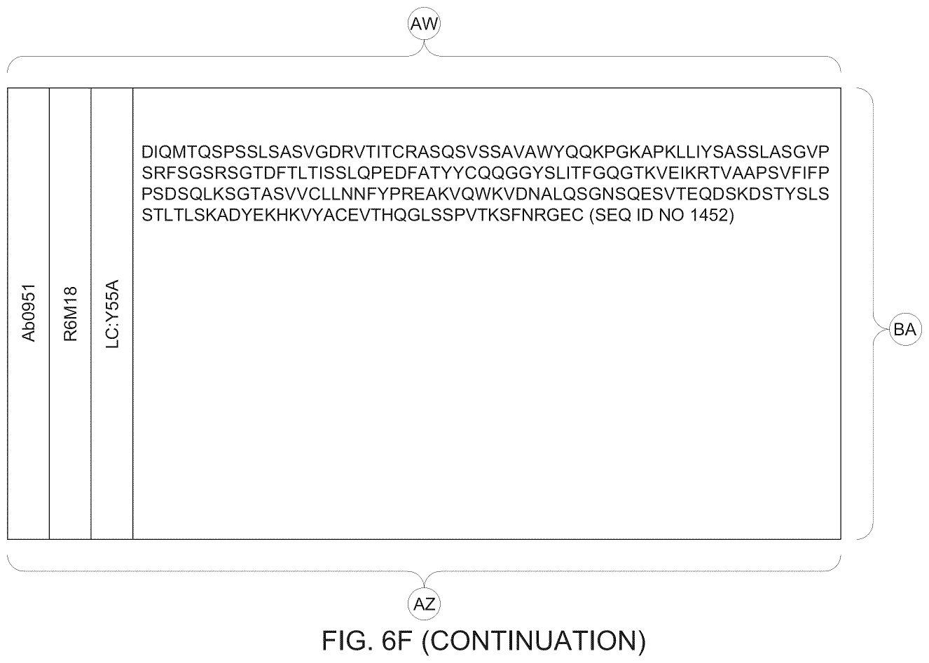

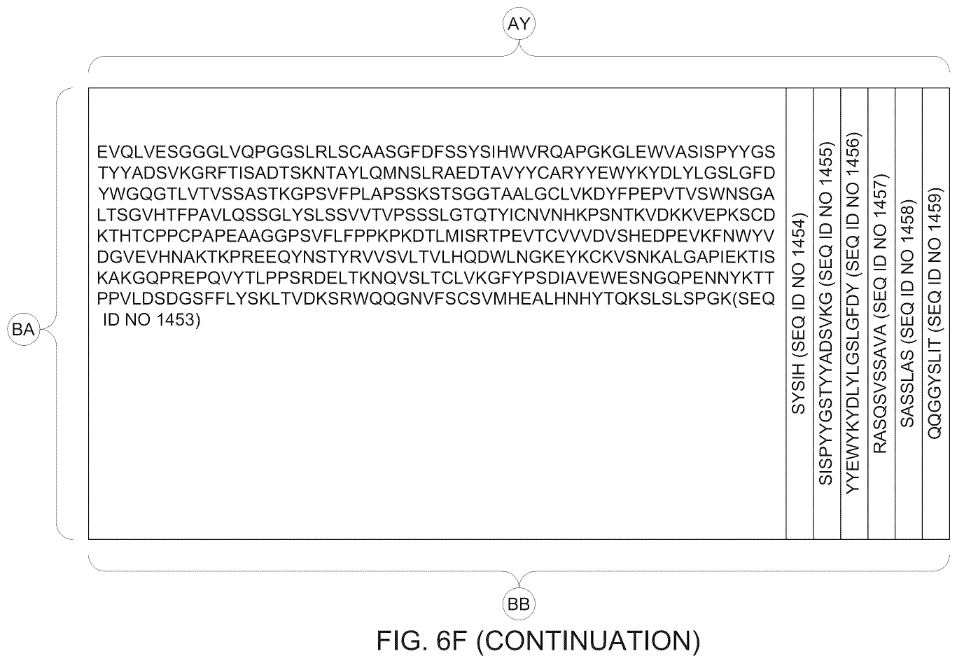

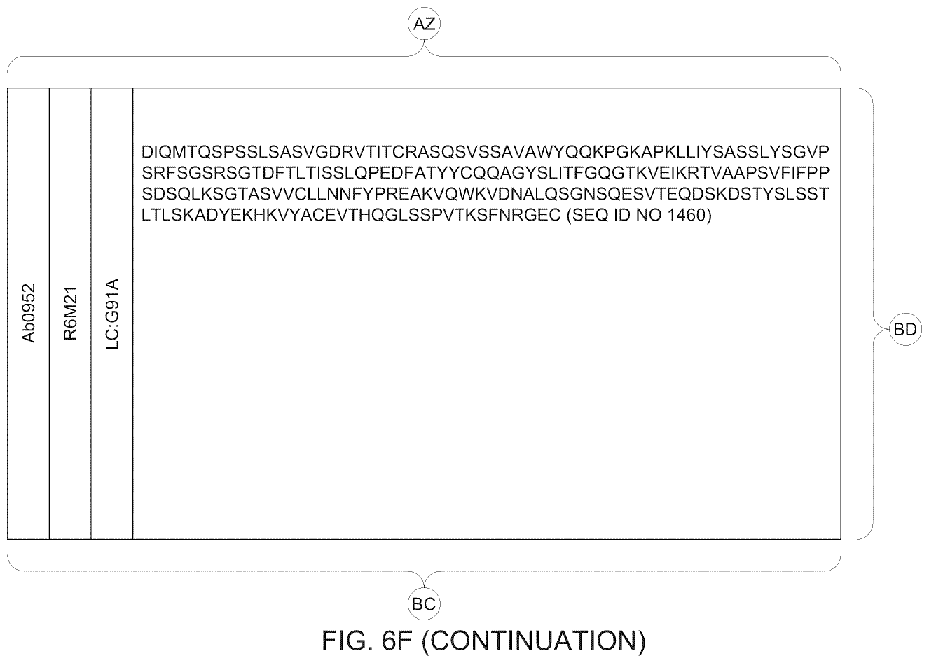

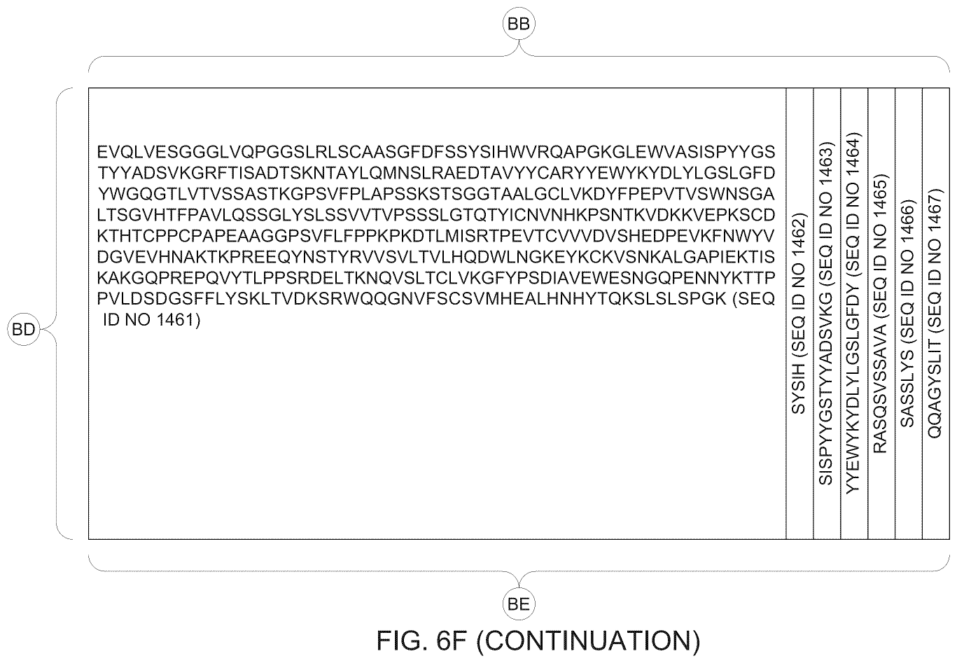

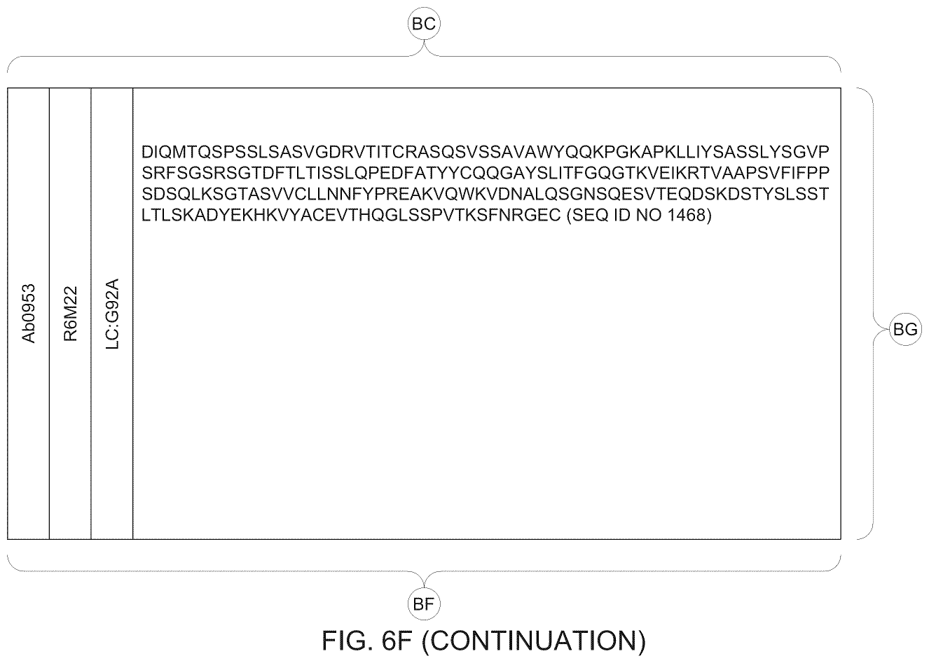

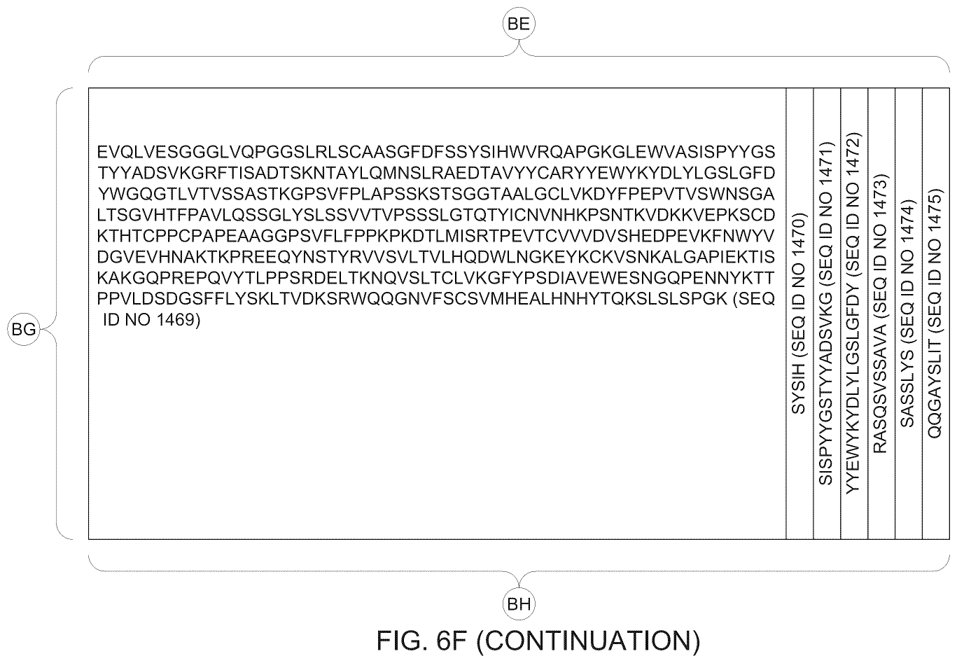

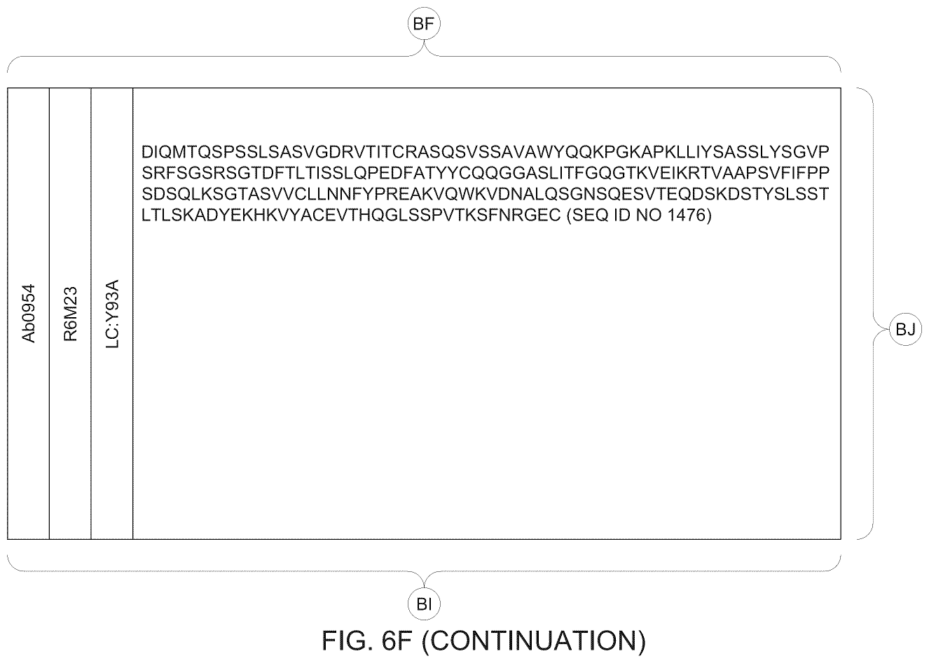

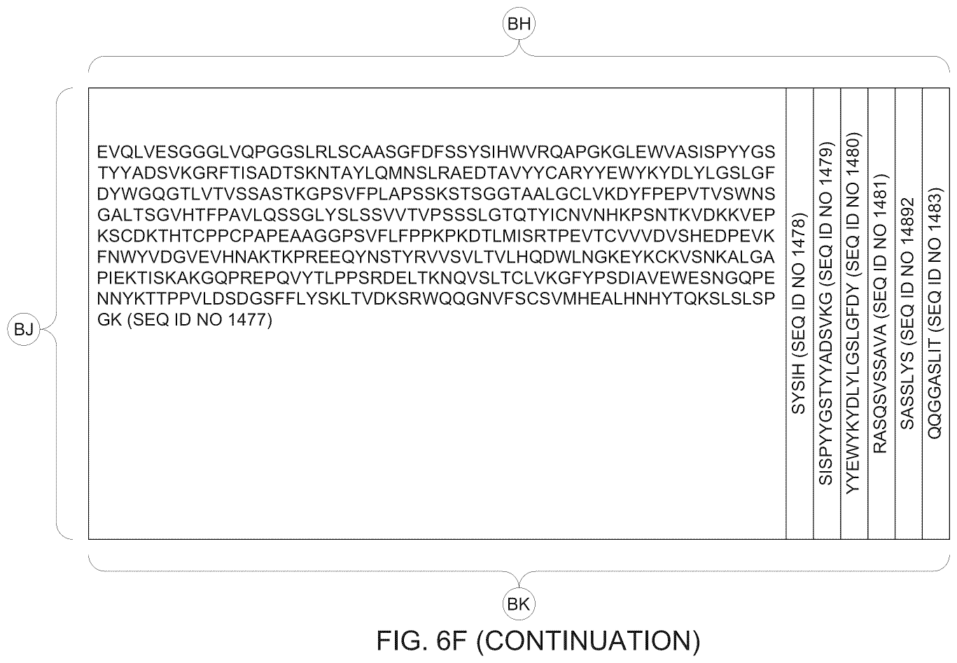

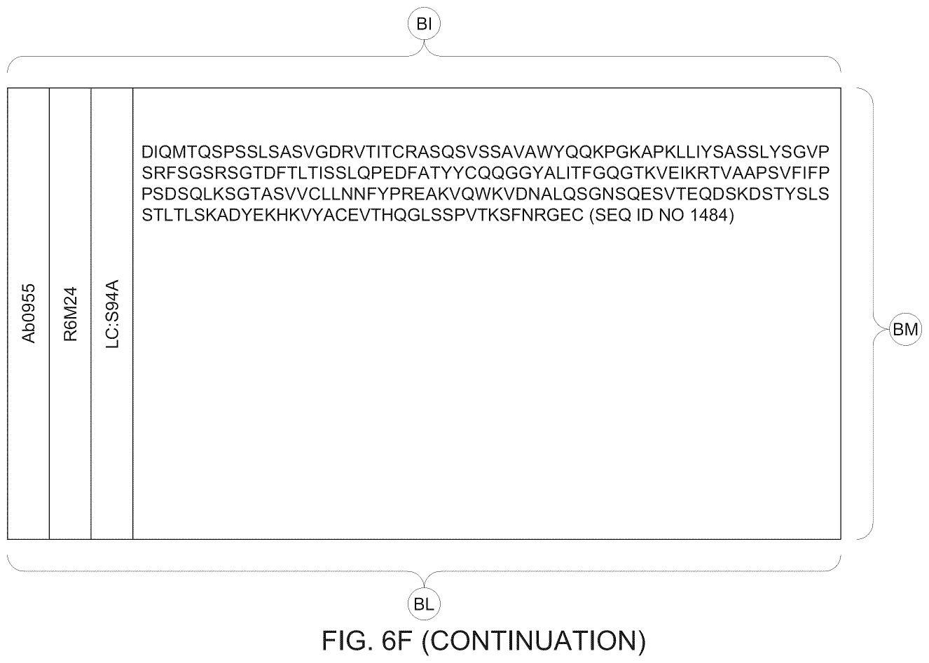

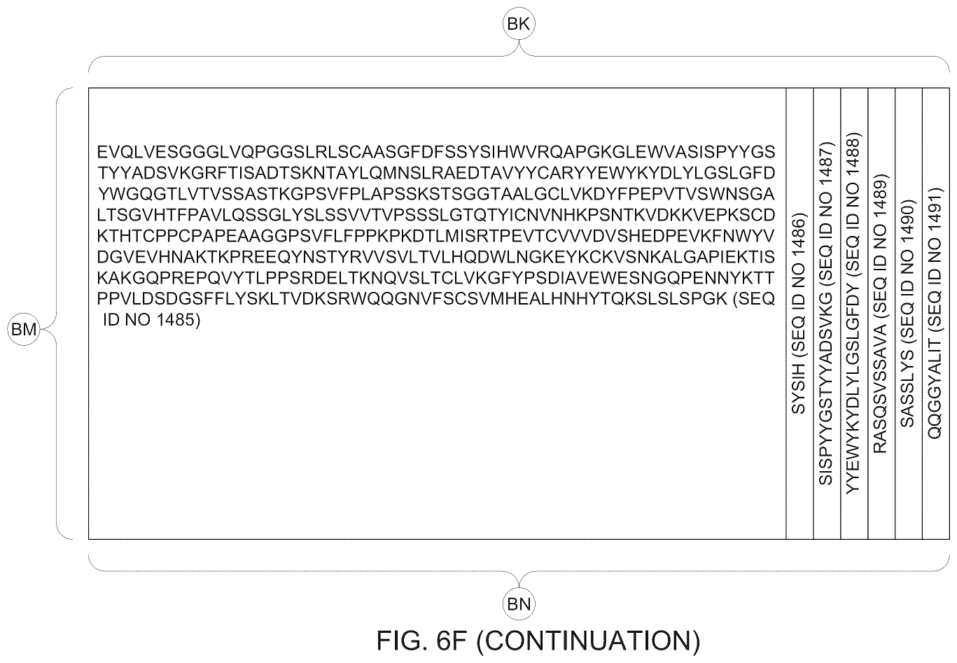

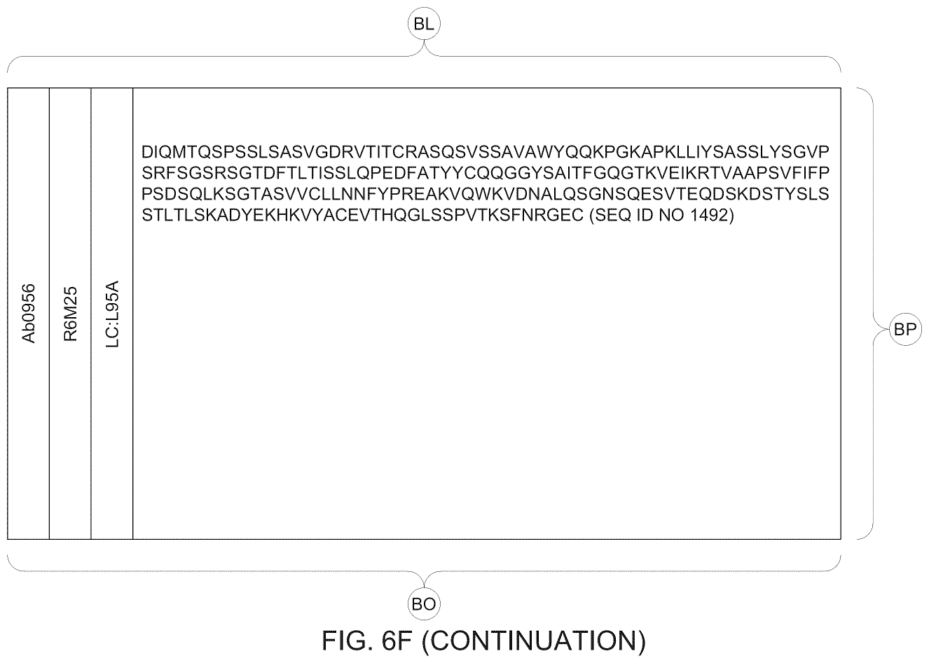

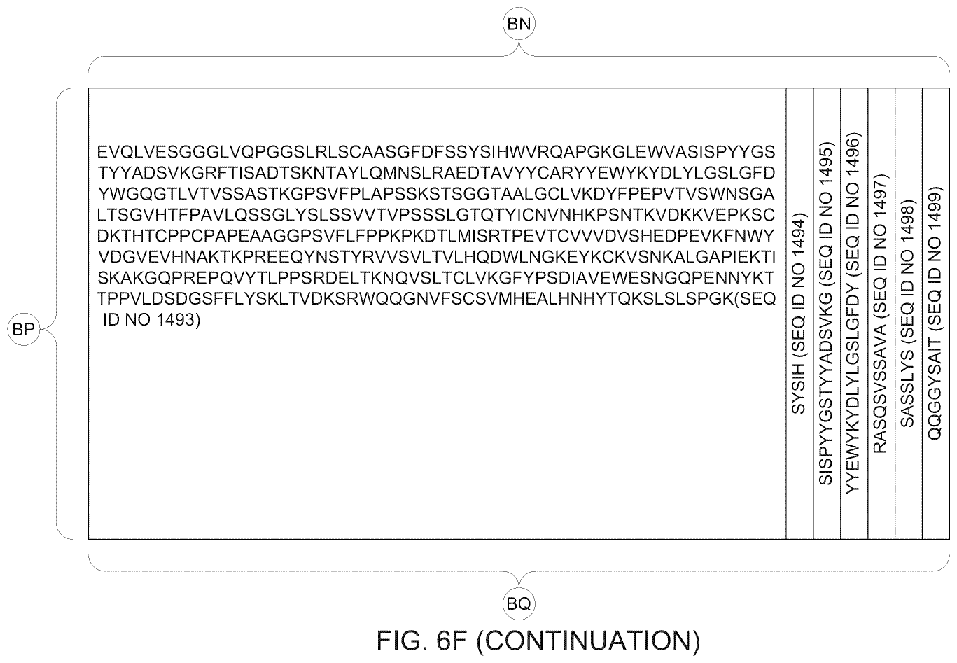

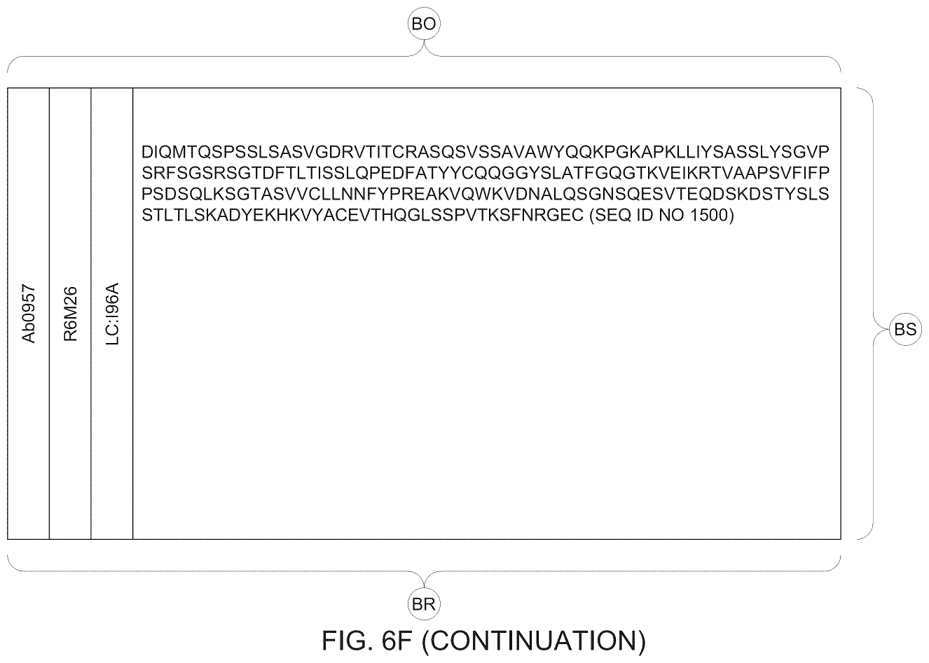

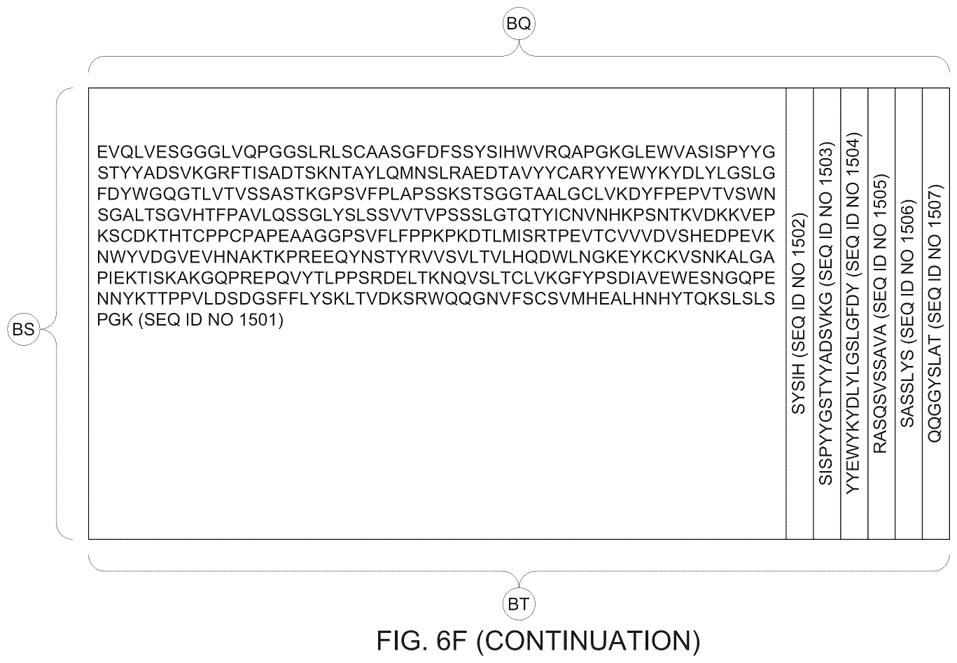

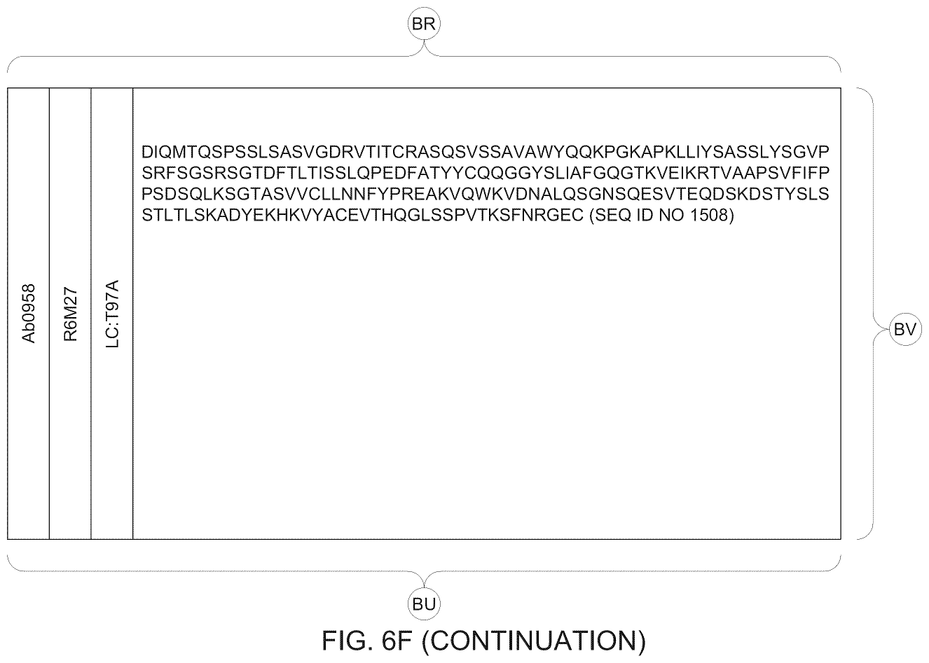

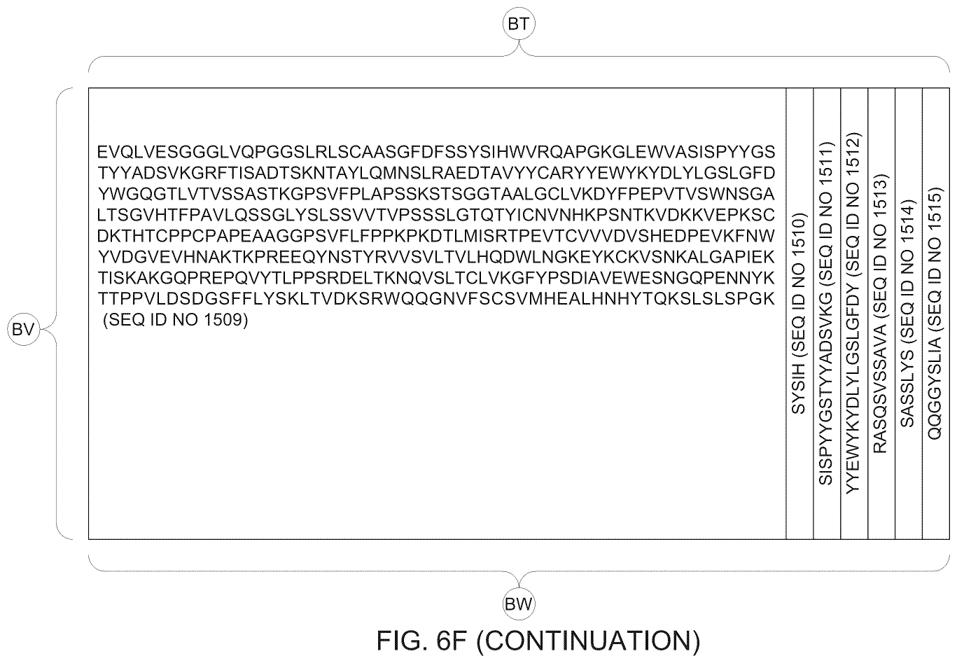

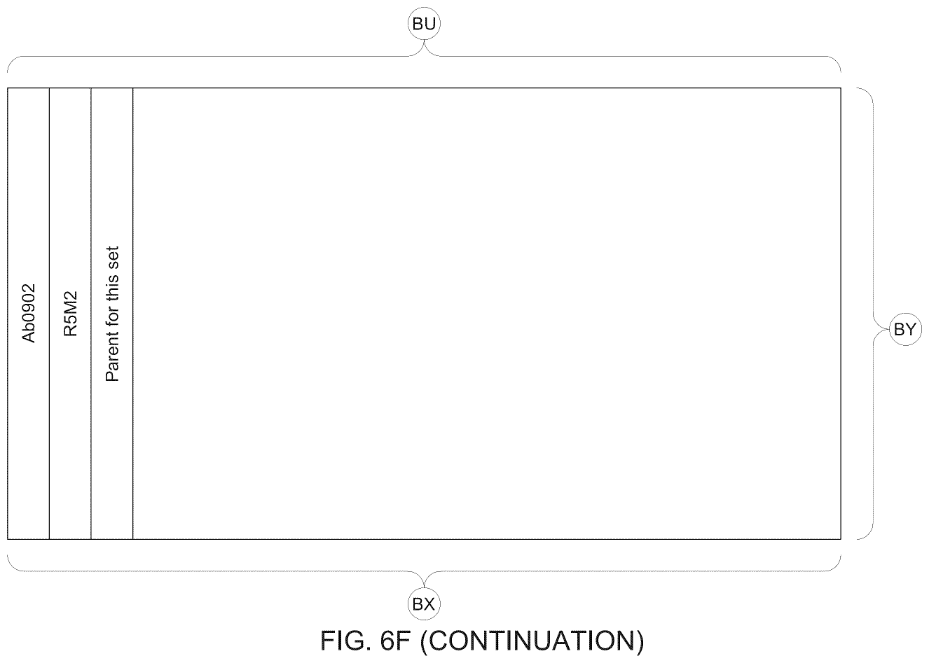

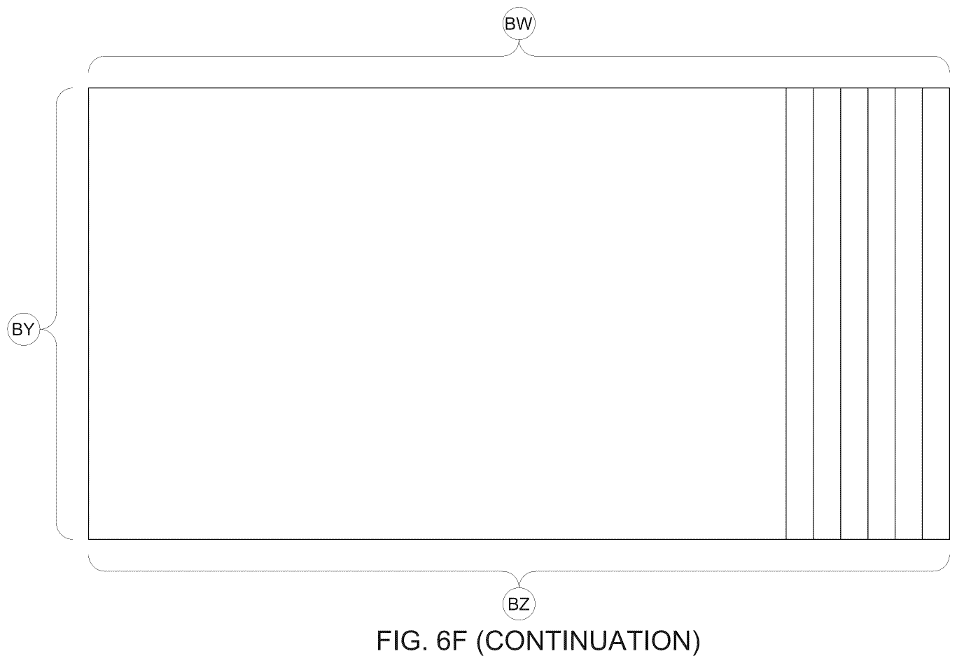

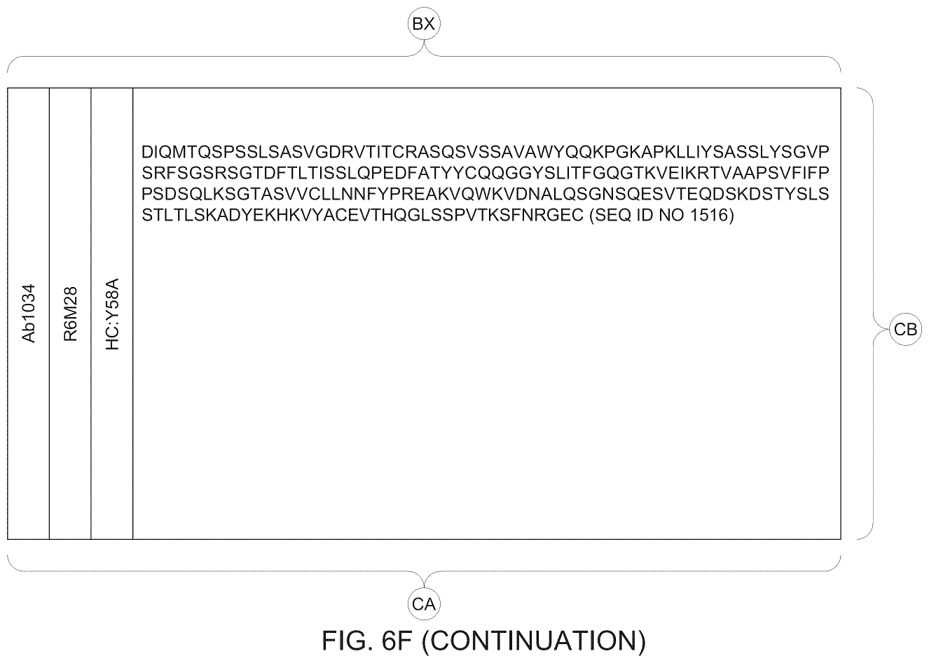

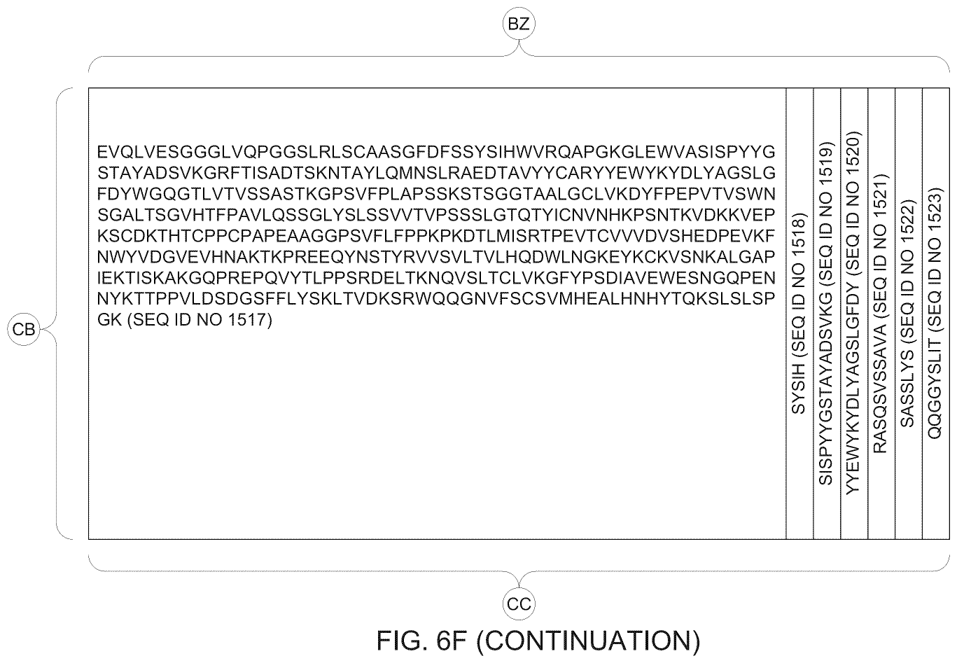

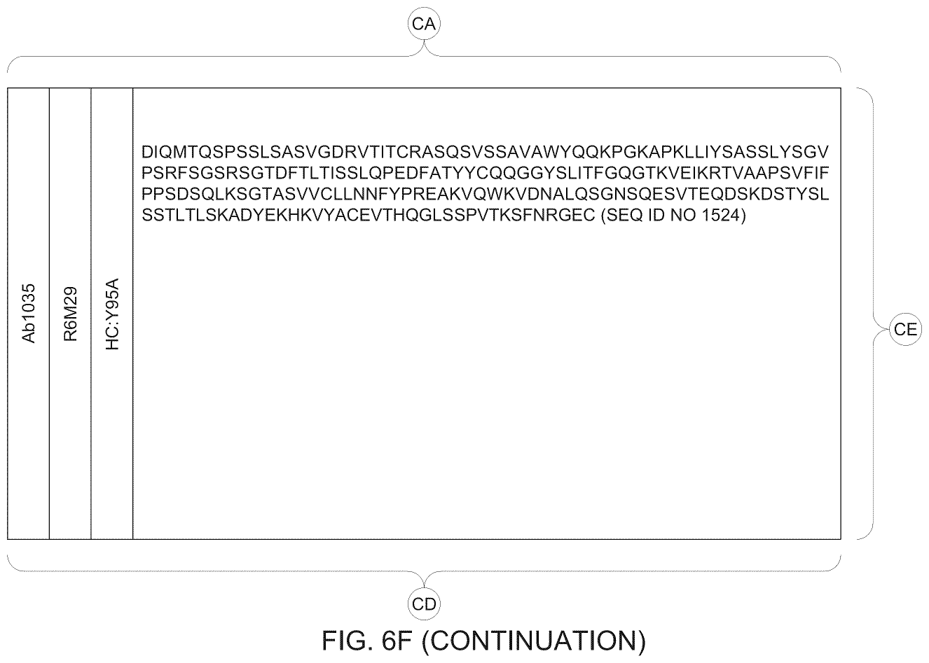

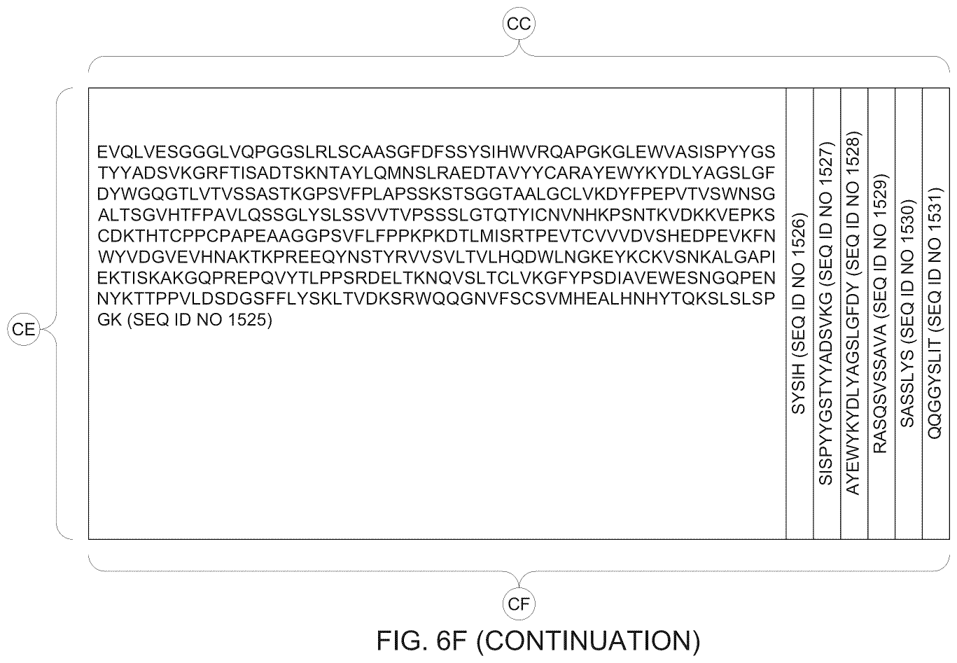

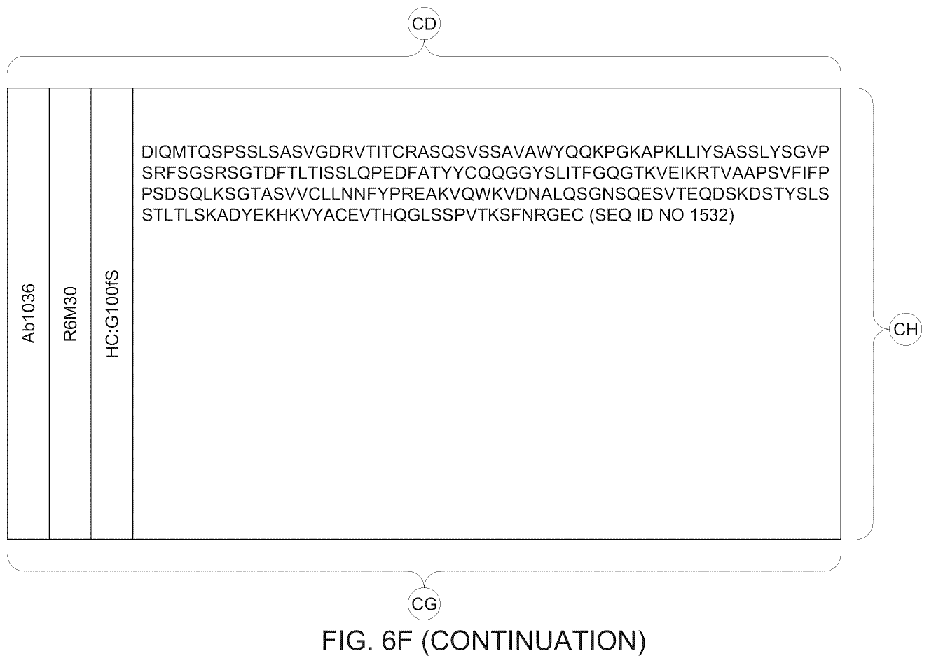

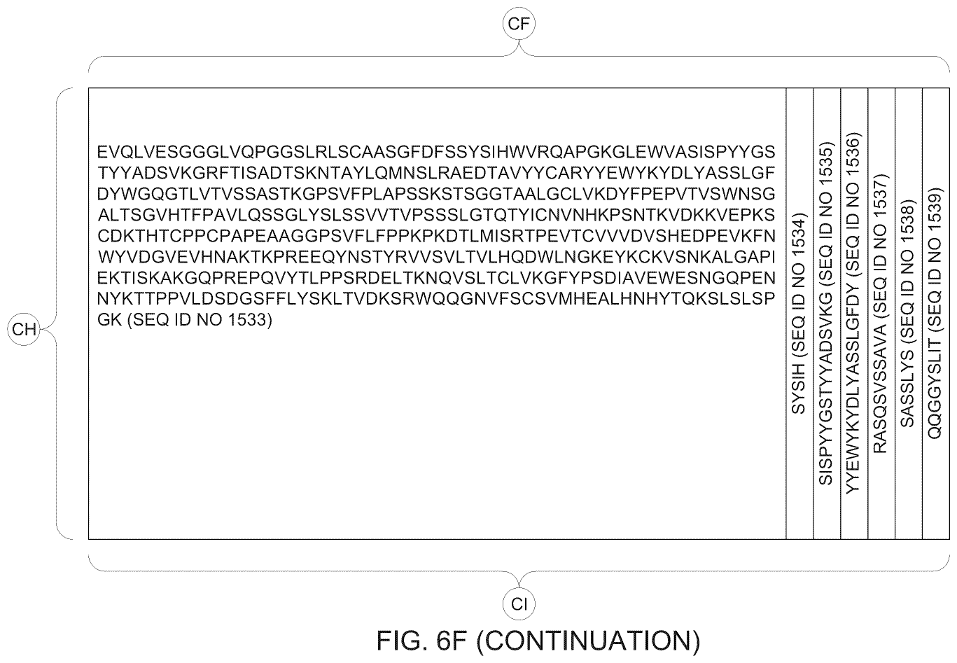

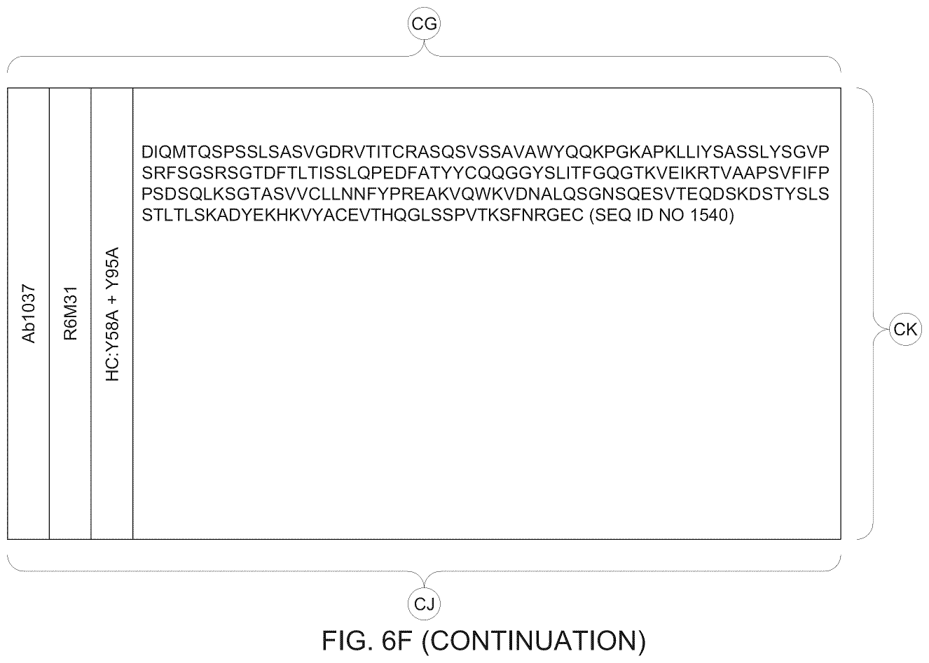

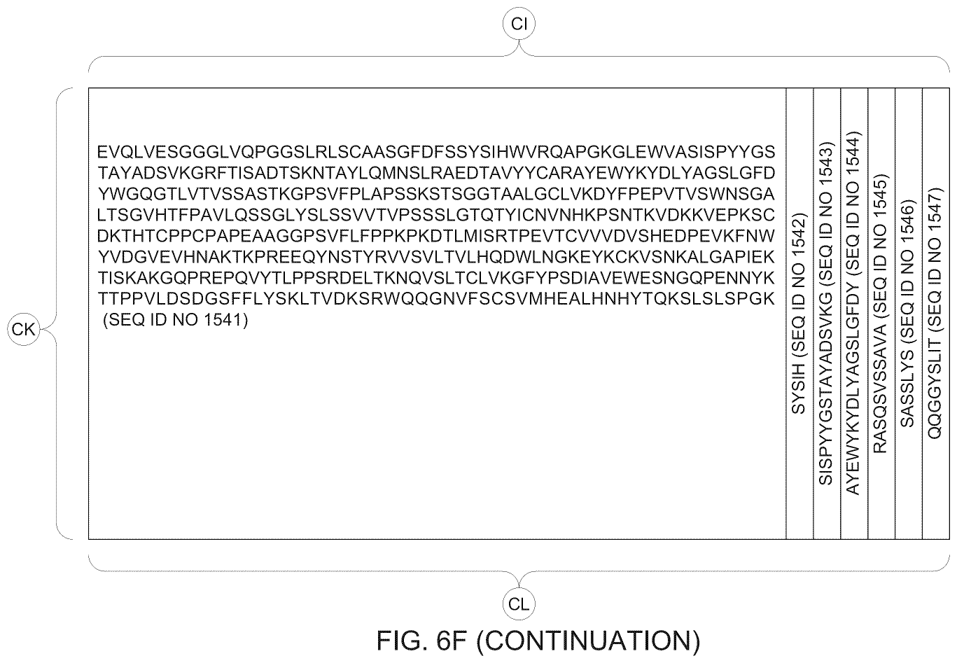

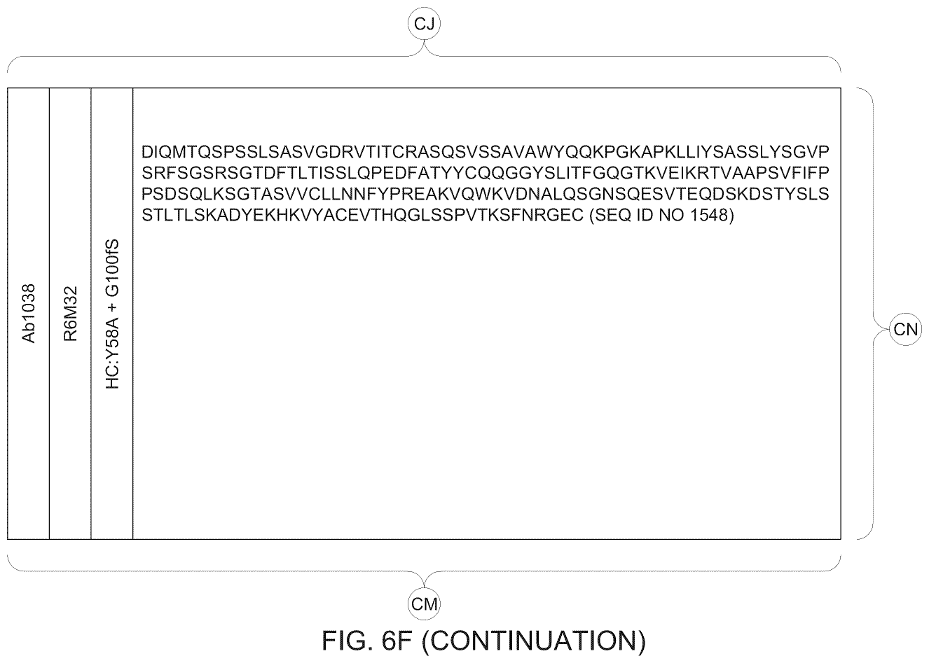

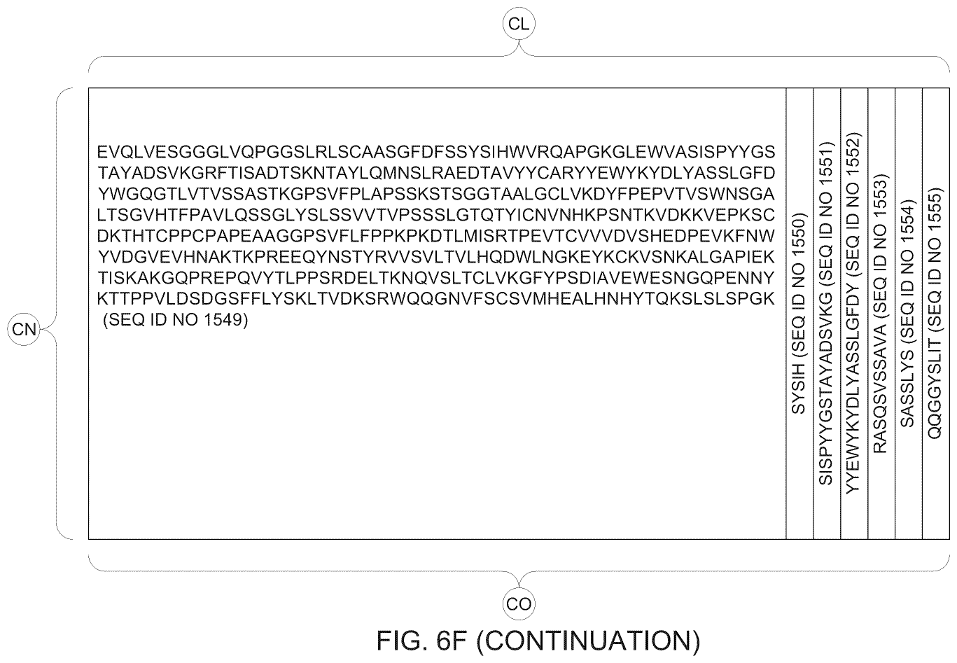

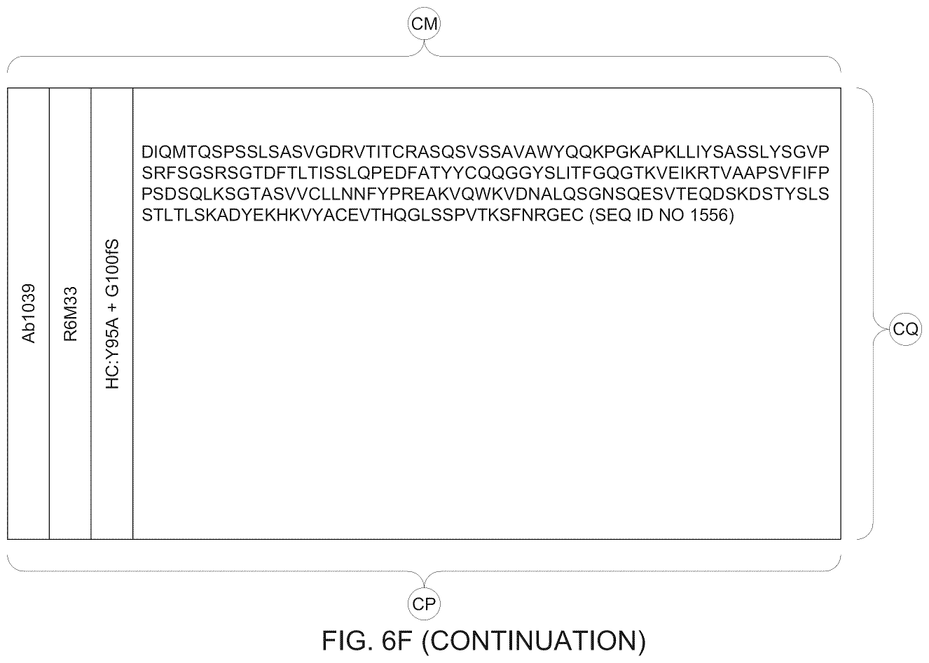

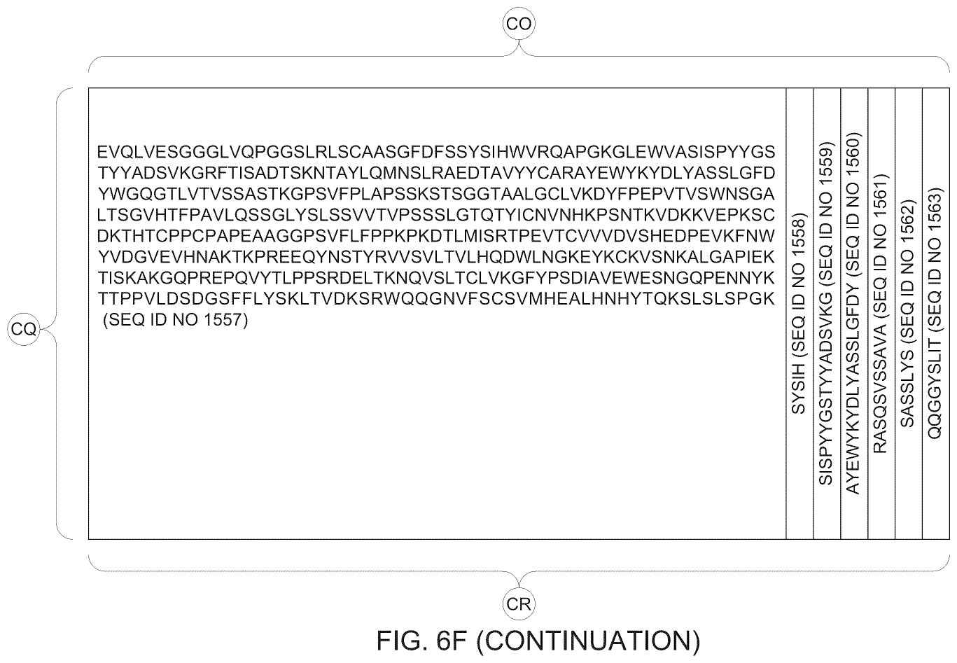

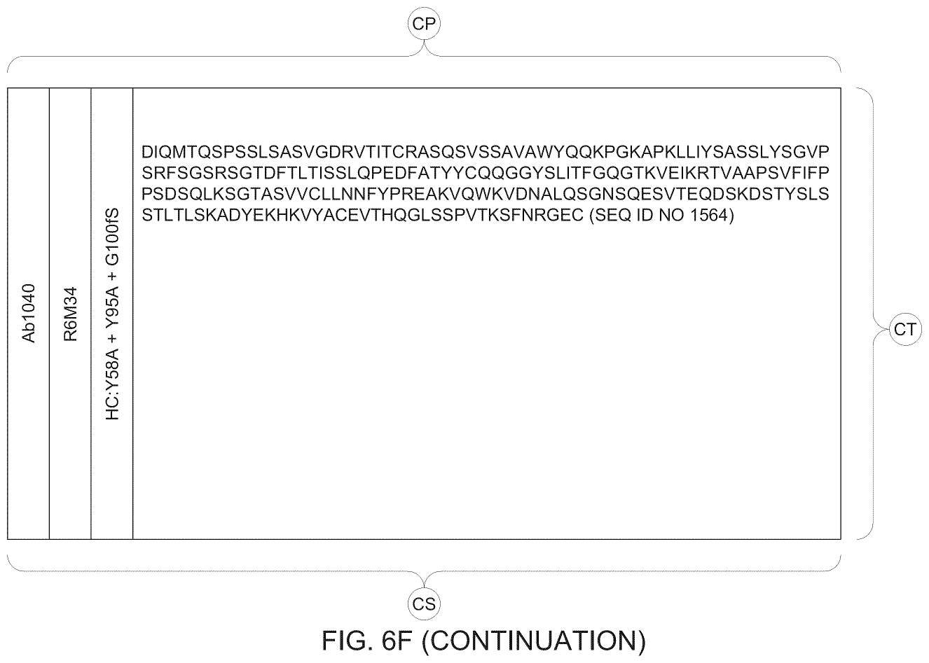

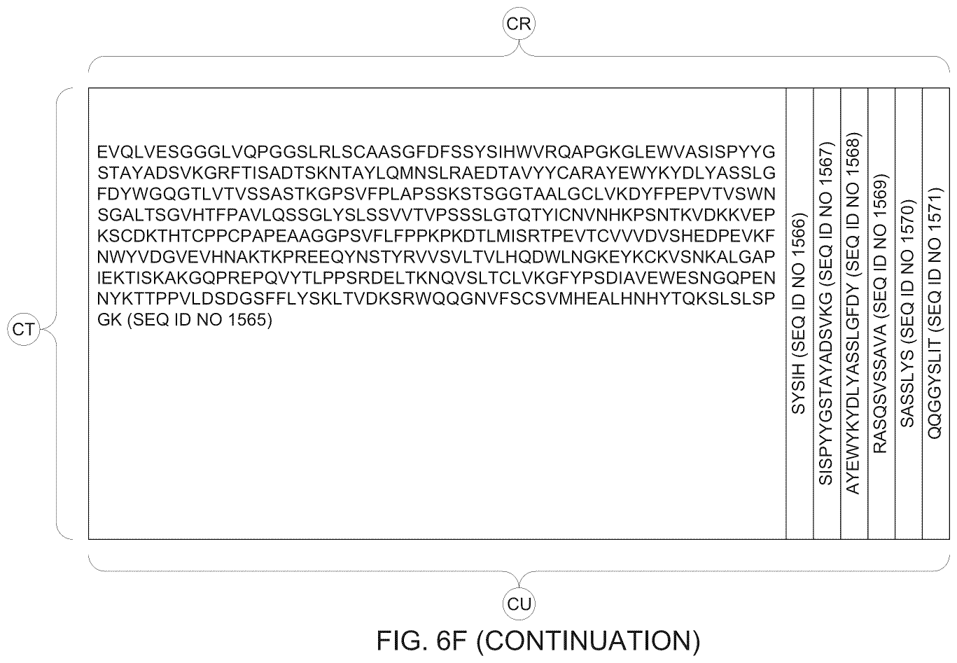

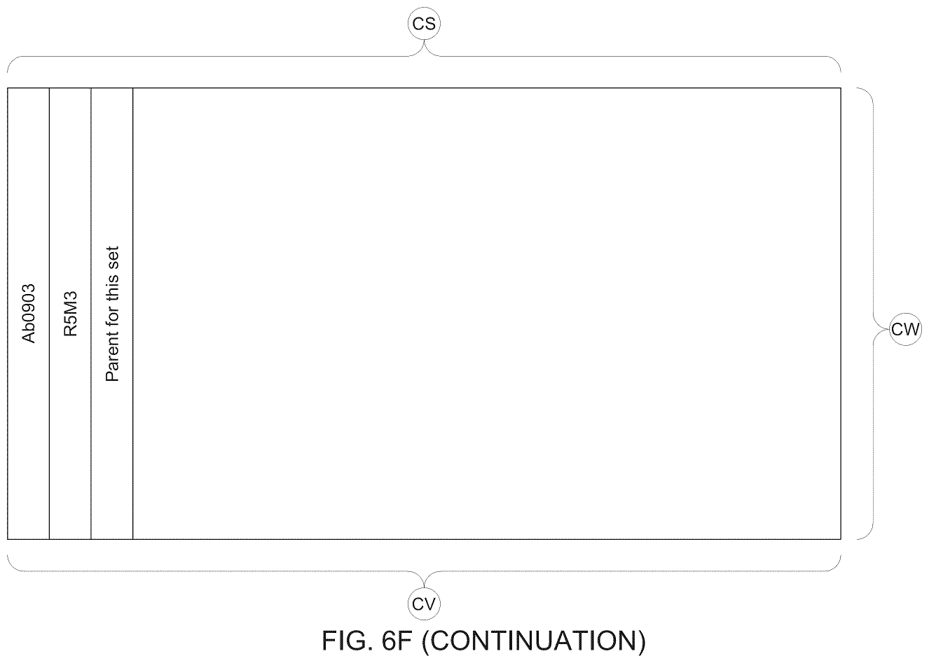

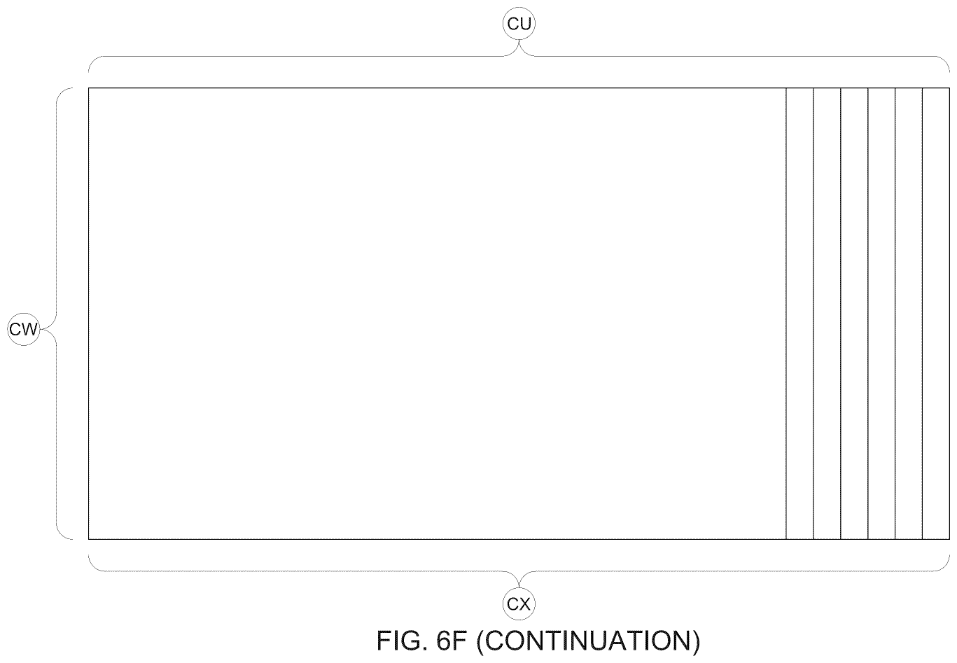

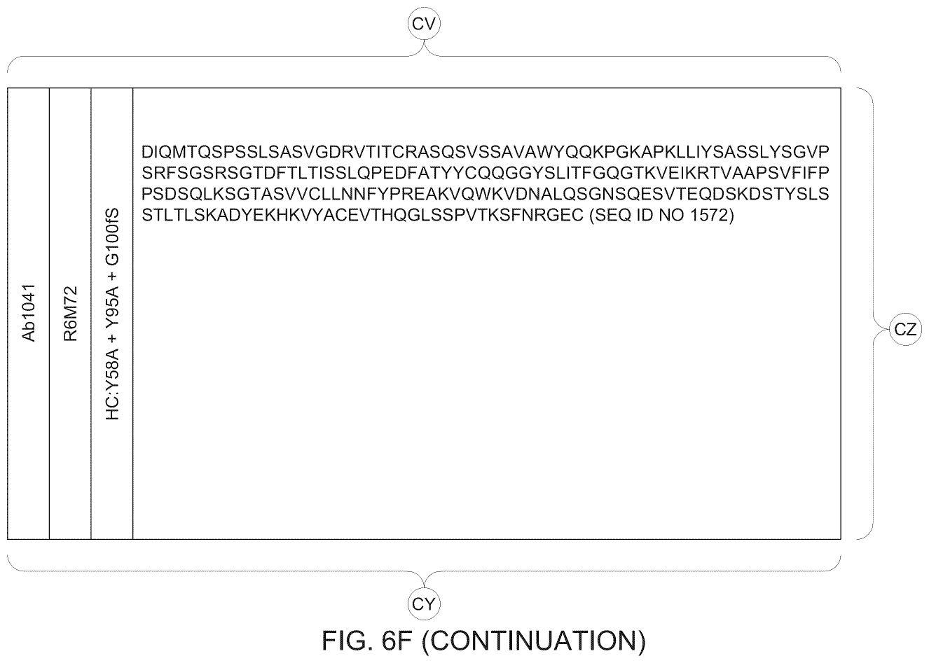

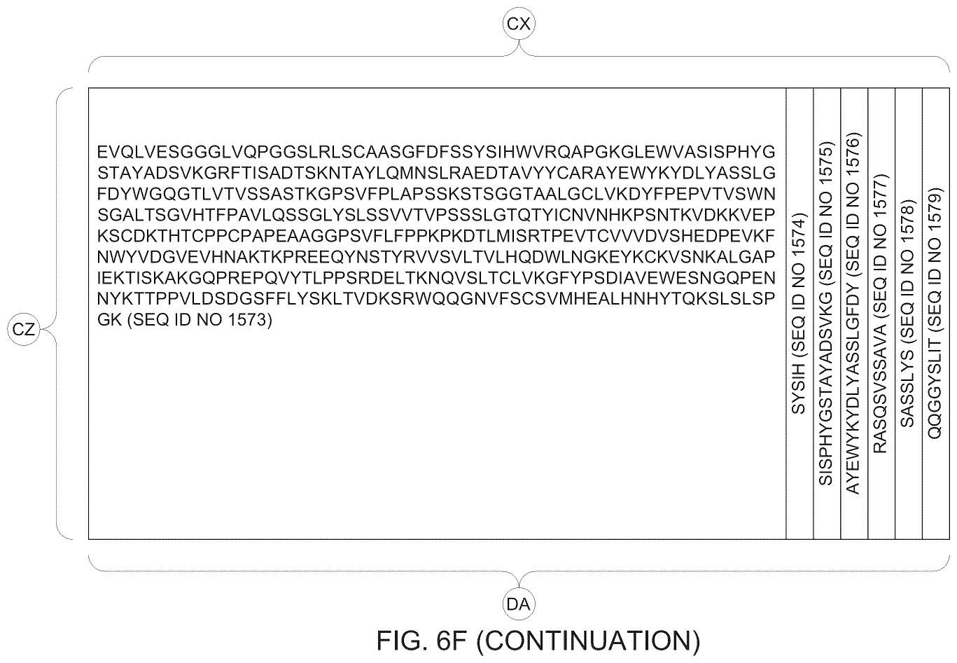

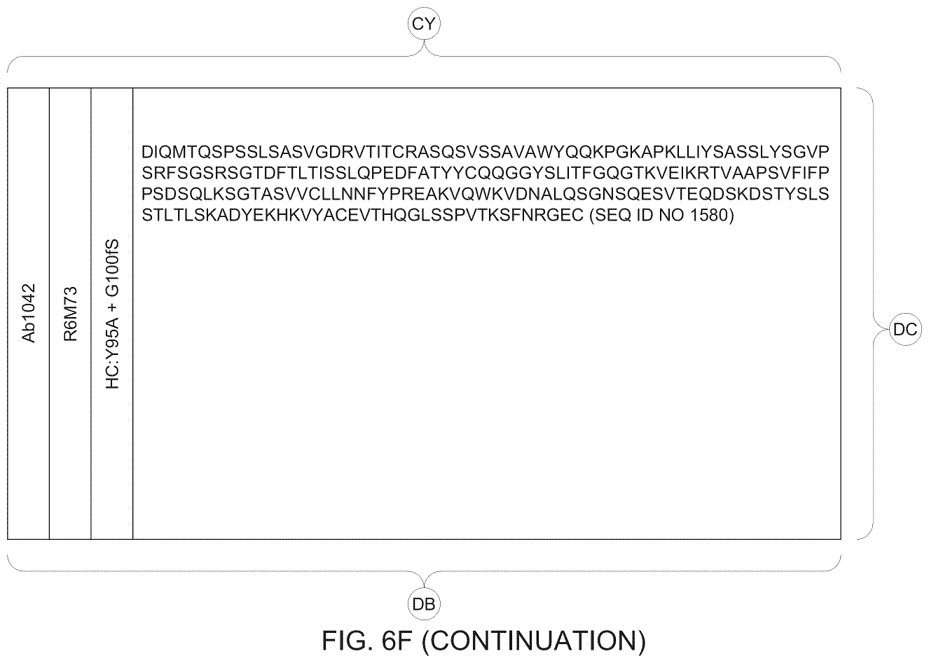

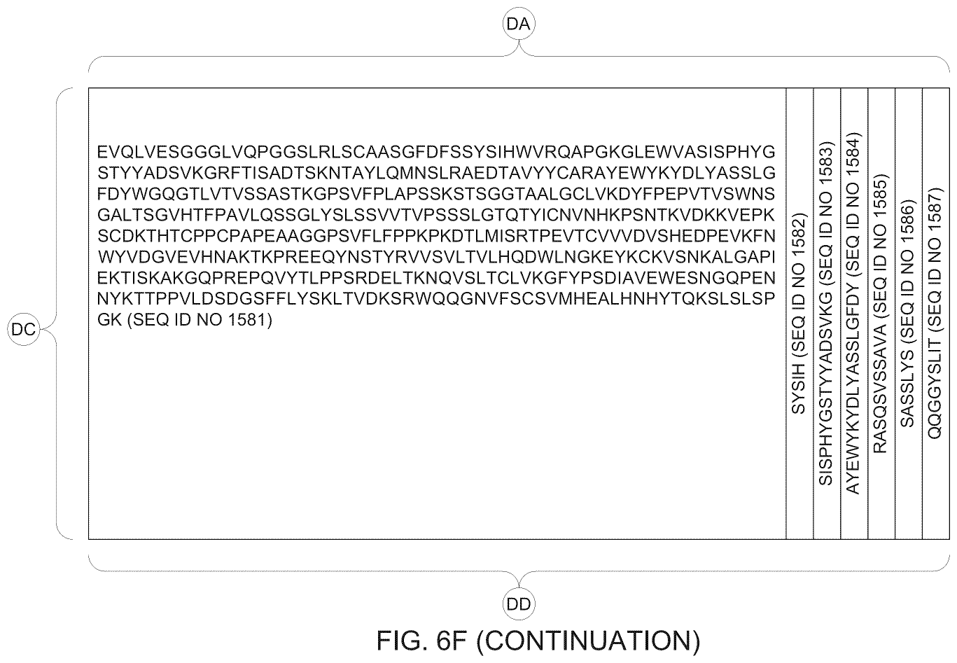

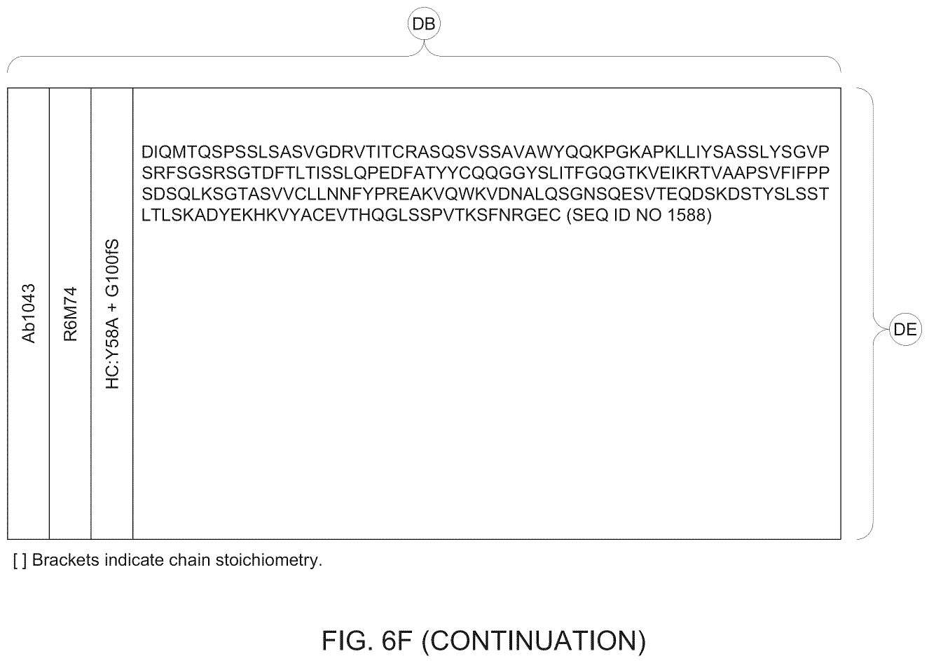

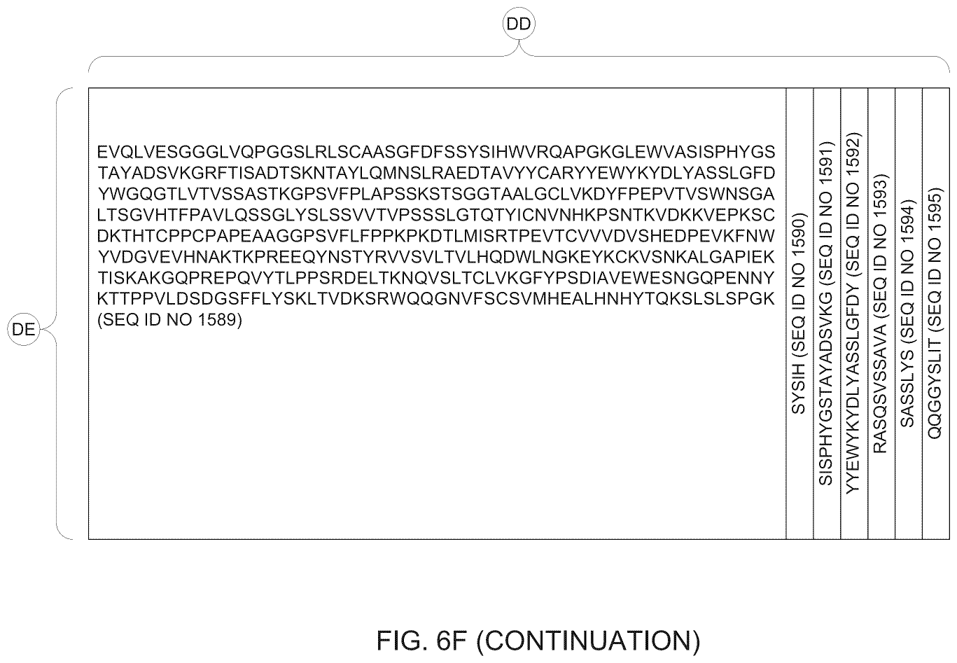

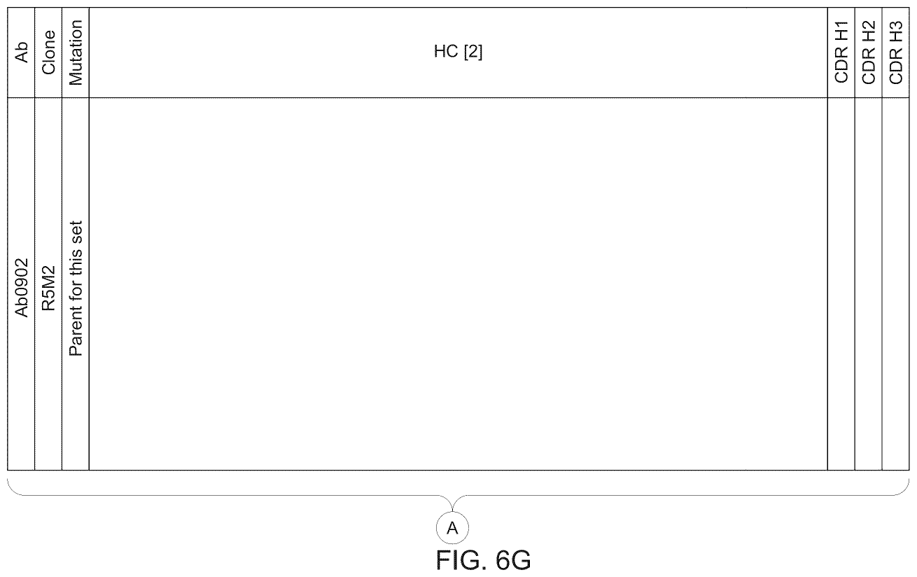

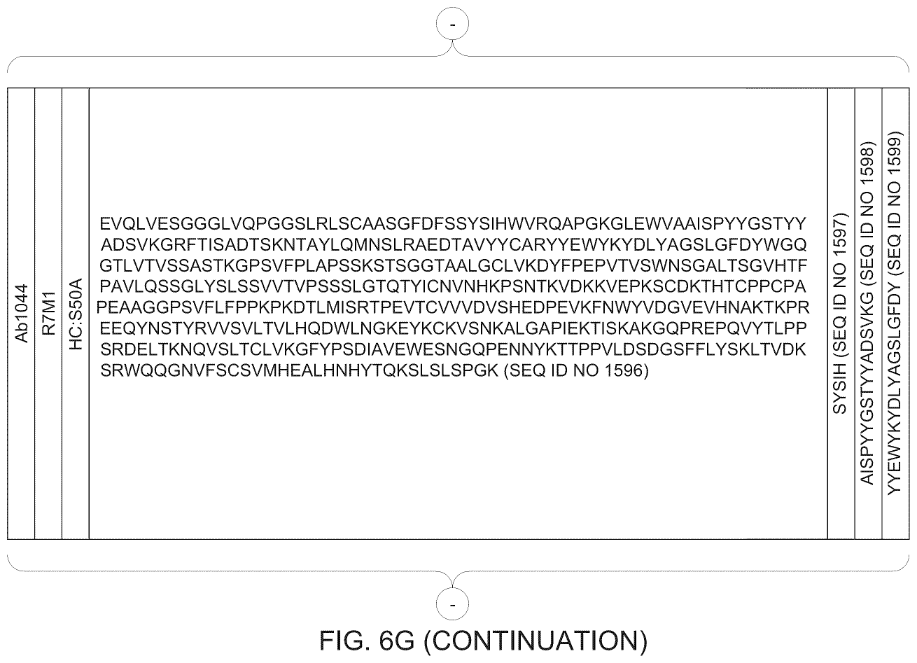

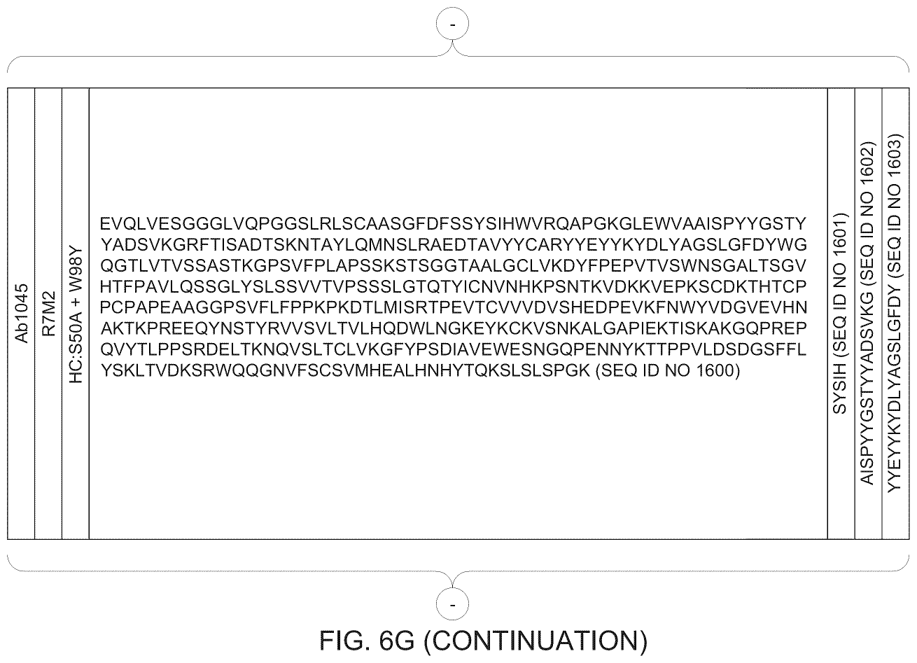

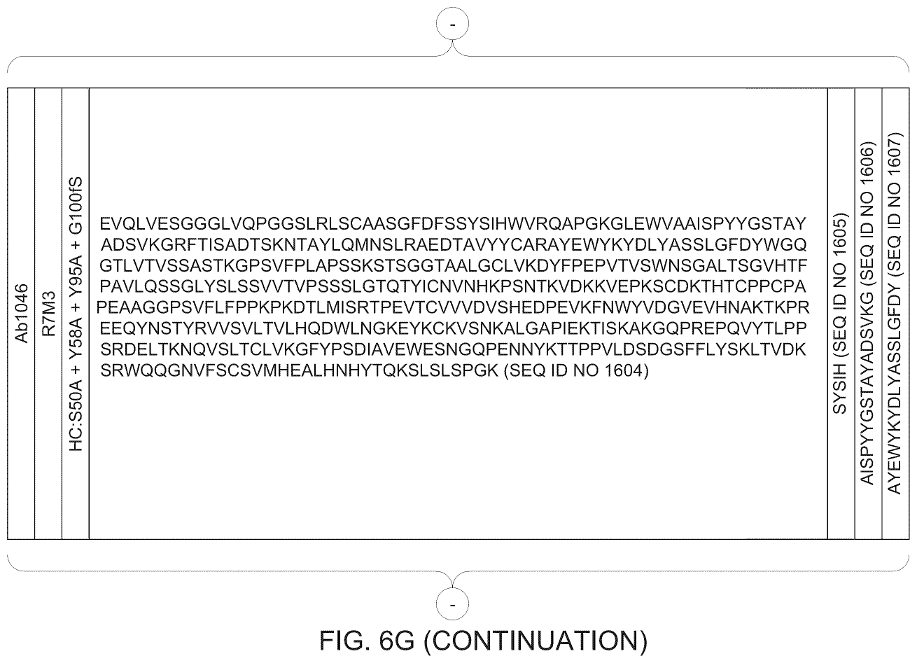

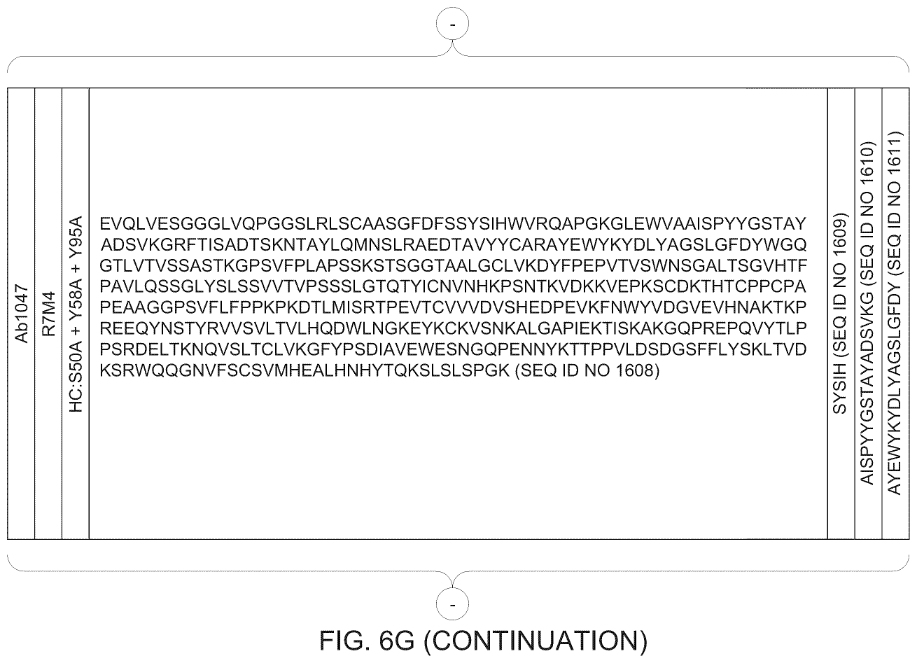

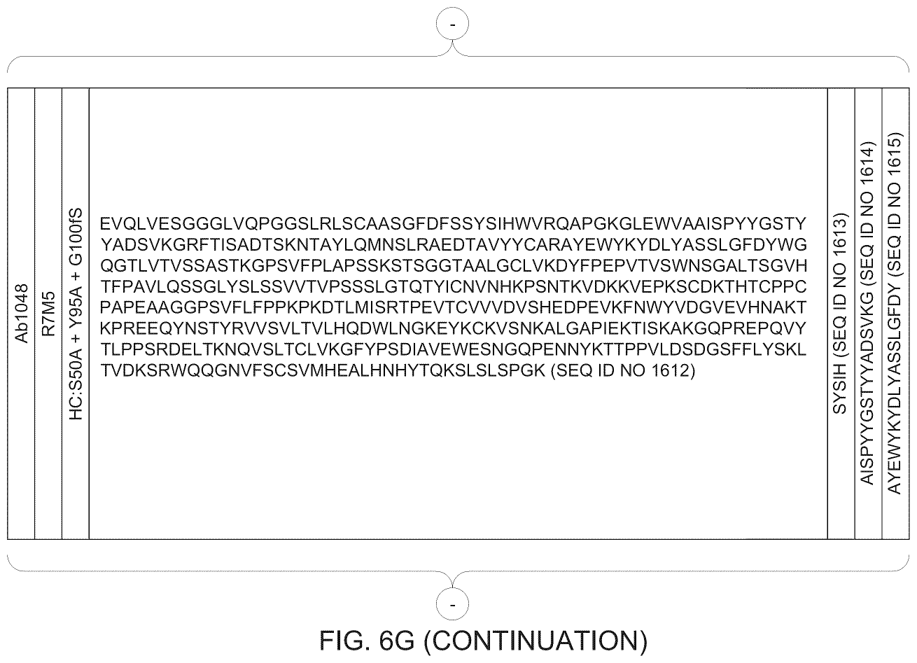

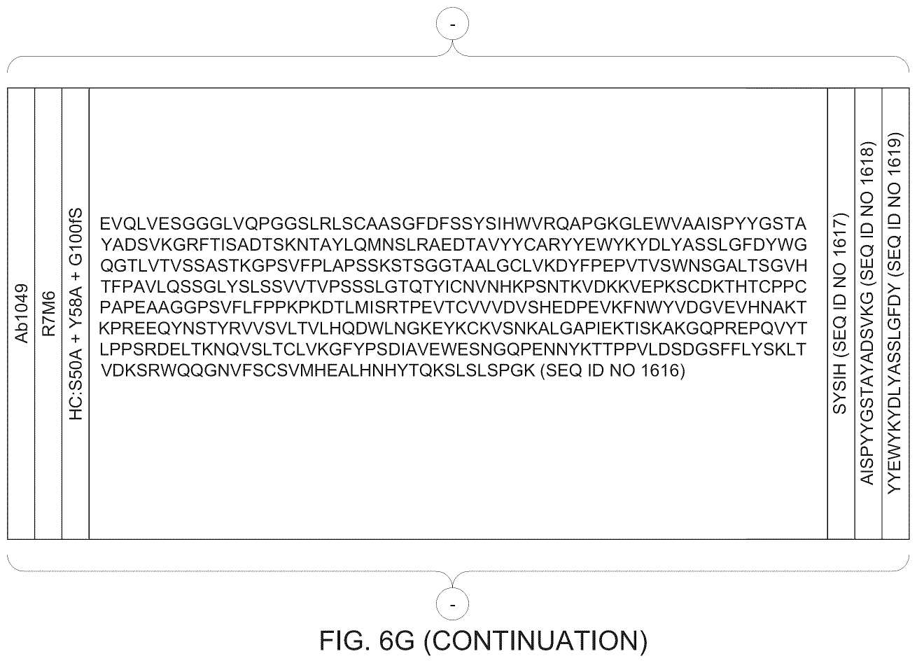

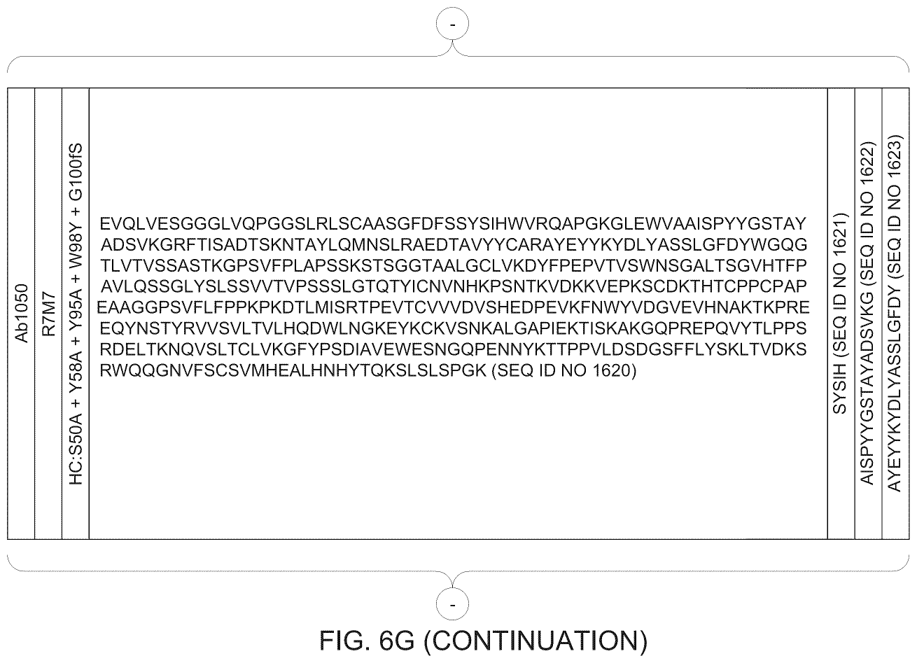

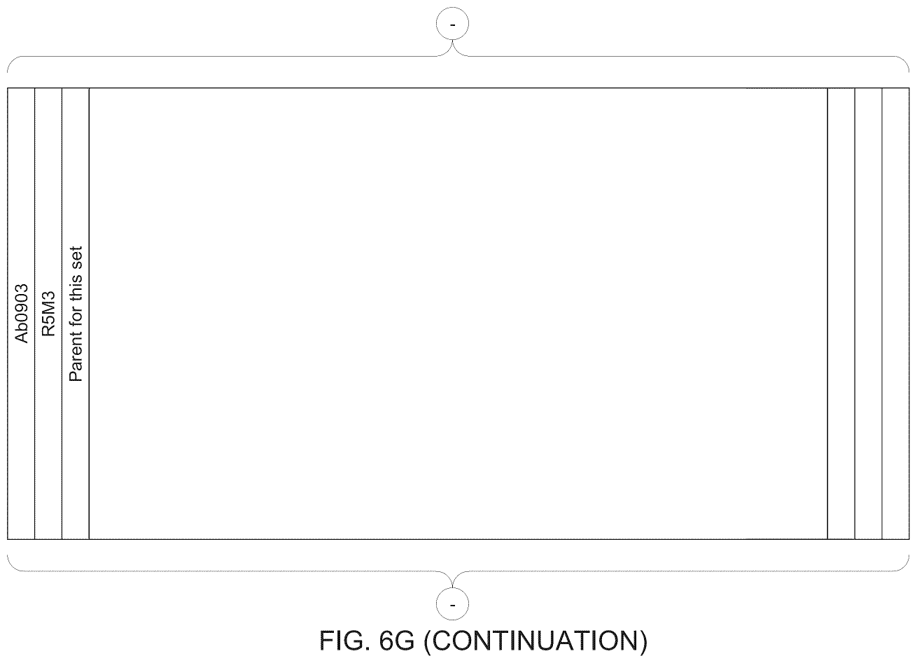

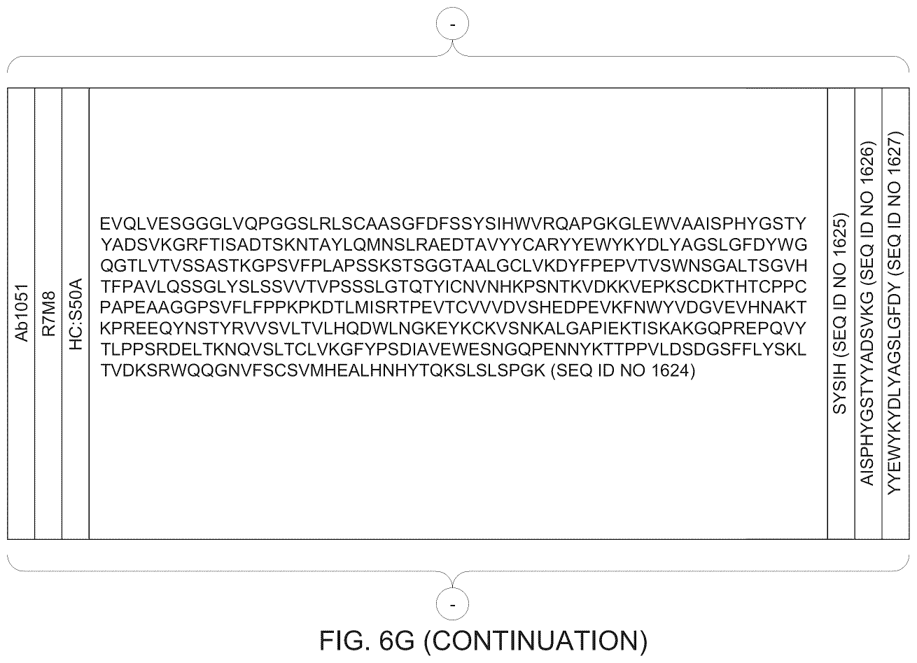

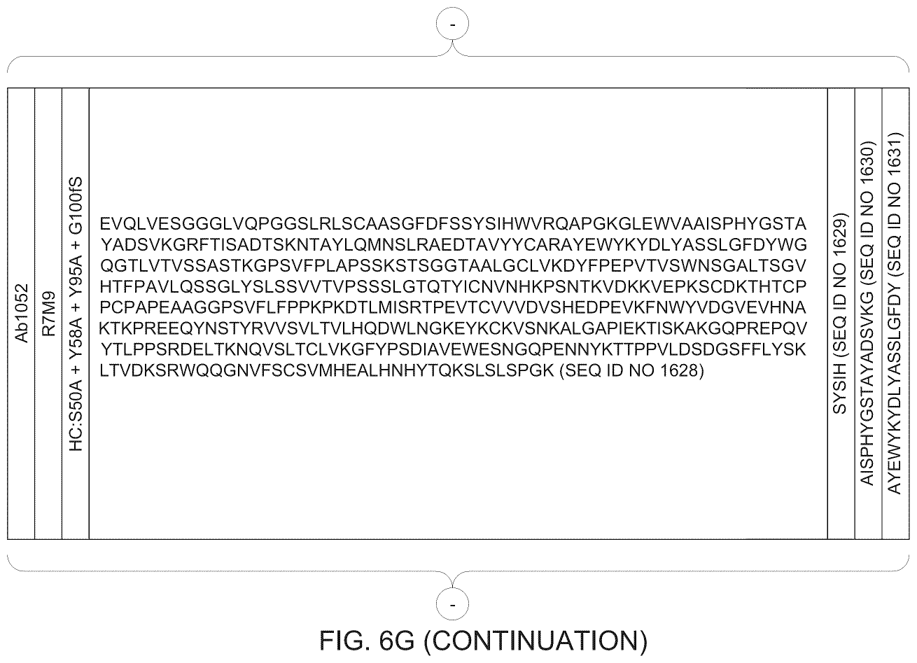

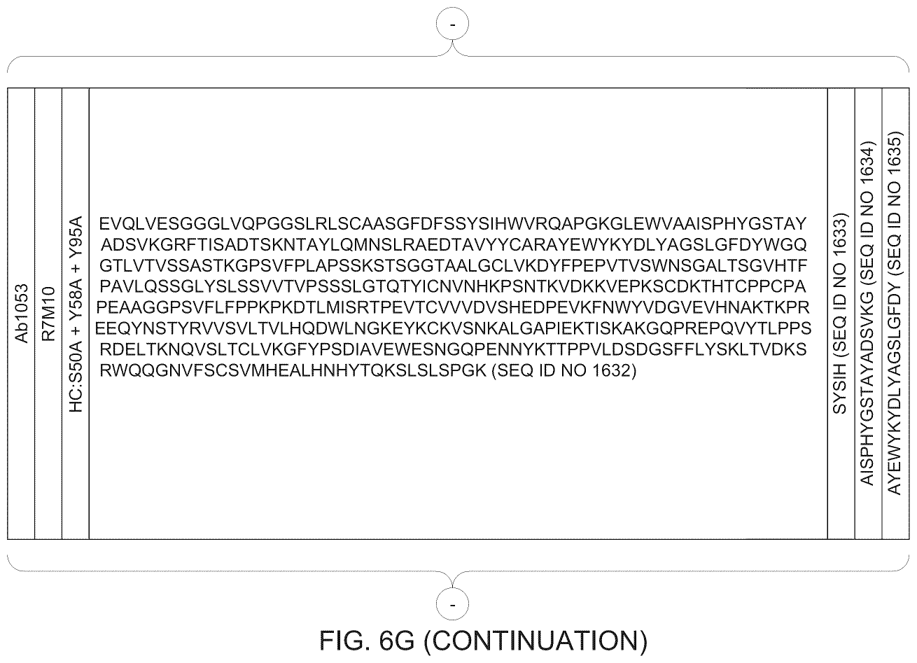

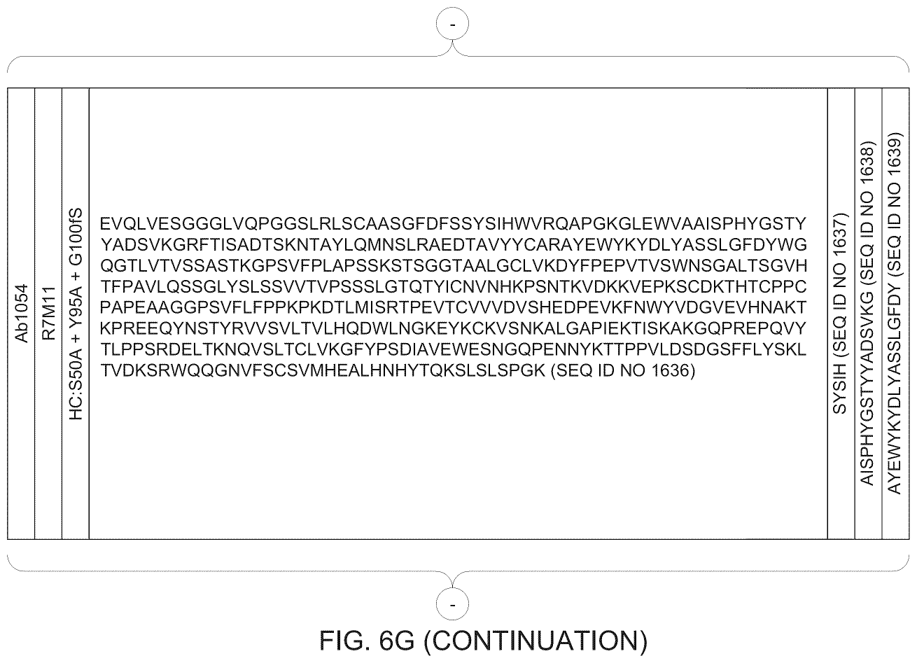

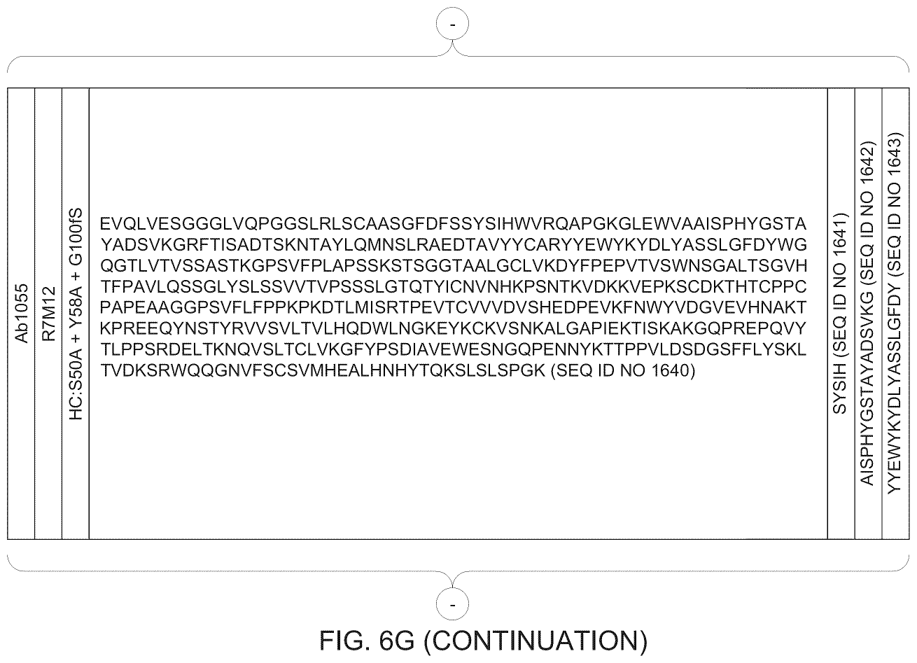

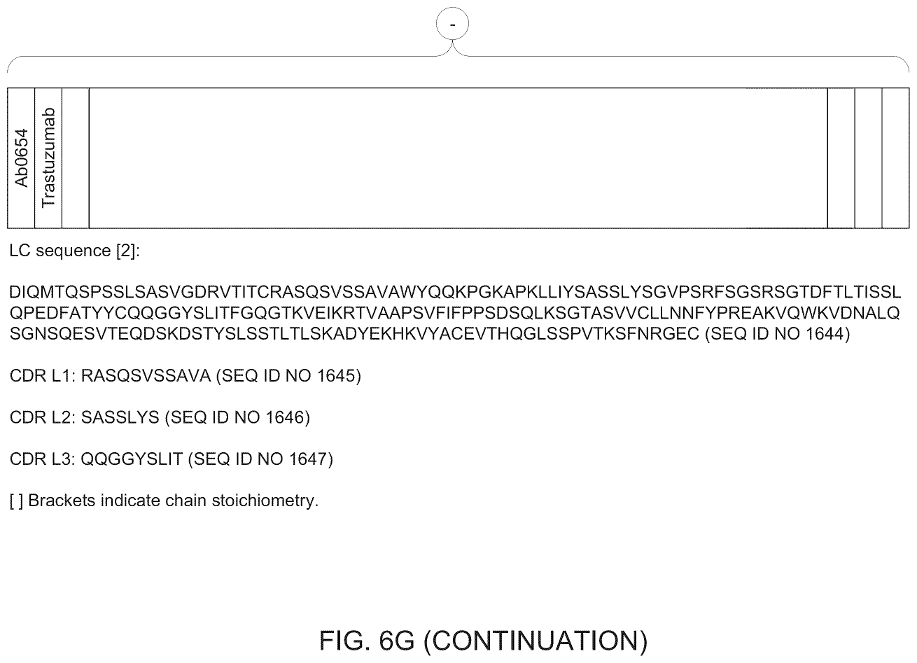

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 6A-6G optionally with one or more mutations. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 6A-6G optionally with one or more mutations selected from group consisting of mutations corresponding to one or more positions 50, 58, 95, 98 and 100 in the heavy chain domain of Ab1091 of FIGS. 6A-6G. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 6A-6G.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from Ab0272, Ab0388, Ab0389, Ab0390, Ab0391, Ab0392, Ab0393, Ab0394, Ab0395, Ab0396, Ab0397, Ab0398, Ab0399, Ab0400, Ab0401, Ab0402, Ab0403, Ab0404, Ab0405, Ab0406, Ab0407, Ab0408, Ab0409, Ab0410, Ab0411, Ab0412, Ab0413, Ab0414, Ab0443, Ab0445, Ab0446, Ab0450, Ab0452, Ab0459, Ab0468, Ab0471, Ab0472, Ab0595, Ab0596, Ab0597, Ab0600, Ab0601, Ab0602, Ab0603, Ab0604, Ab0608, Ab0609, Ab0610, Ab0611, Ab0612, Ab0635, Ab0636, Ab0637, Ab0638, Ab0687, Ab0688, Ab0689, Ab0690, Ab0692, Ab0698, Ab0699, Ab0706, Ab0707, Ab0708, Ab0709, Ab0710, Ab0901, Ab0902, Ab0903, Ab0654, Ab0936, Ab0937, Ab0939, Ab0941, Ab0944, Ab0946, Ab0947, Ab0953, Ab0956, Ab0957, Ab0958, Ab1034, Ab1037, Ab1044, Ab1045, Ab1047, Ab1051, Ab1091 of FIGS. 6A-6G. In some embodiments, the sequences are from Ab0272. In some embodiments, the sequences are from Ab0388. In some embodiments, the sequences are from Ab0389. In some embodiments, the sequences are from Ab0390. In some embodiments, the sequences are from Ab0391. In some embodiments, the sequences are from Ab0392. In some embodiments, the sequences are from Ab0393. In some embodiments, the sequences are from Ab0394. In some embodiments, the sequences are from Ab0395. In some embodiments, the sequences are from Ab0396. In some embodiments, the sequences are from Ab0397. In some embodiments, the sequences are from Ab0398. In some embodiments, the sequences are from Ab0399. In some embodiments, the sequences are from Ab0400. In some embodiments, the sequences are from Ab0401. In some embodiments, the sequences are from Ab0402. In some embodiments, the sequences are from Ab0403. In some embodiments, the sequences are from Ab0404. In some embodiments, the sequences are from Ab0405. In some embodiments, the sequences are from Ab0406. In some embodiments, the sequences are from Ab0407. In some embodiments, the sequences are from Ab0408. In some embodiments, the sequences are from Ab0409. In some embodiments, the sequences are from Ab0410. In some embodiments, the sequences are from Ab0411. In some embodiments, the sequences are from Ab0412. In some embodiments, the sequences are from Ab0413. In some embodiments, the sequences are from Ab0414. In some embodiments, the sequences are from Ab0443. In some embodiments, the sequences are from Ab0445. In some embodiments, the sequences are from Ab0446. In some embodiments, the sequences are from Ab0450. In some embodiments, the sequences are from Ab0452. In some embodiments, the sequences are from Ab0459. In some embodiments, the sequences are from Ab0468. In some embodiments, the sequences are from Ab0471. In some embodiments, the sequences are from Ab0472. In some embodiments, the sequences are from Ab0595. In some embodiments, the sequences are from Ab0596. In some embodiments, the sequences are from Ab0597. In some embodiments, the sequences are from Ab0600. In some embodiments, the sequences are from Ab0601. In some embodiments, the sequences are from Ab0602. In some embodiments, the sequences are from Ab0603. In some embodiments, the sequences are from Ab0604. In some embodiments, the sequences are from Ab0608. In some embodiments, the sequences are from Ab0609. In some embodiments, the sequences are from Ab0610. In some embodiments, the sequences are from Ab0611. In some embodiments, the sequences are from Ab0612. In some embodiments, the sequences are from Ab0635. In some embodiments, the sequences are from Ab0636. In some embodiments, the sequences are from Ab0637. In some embodiments, the sequences are from Ab0638. In some embodiments, the sequences are from Ab0687. In some embodiments, the sequences are from Ab0688. In some embodiments, the sequences are from Ab0689. In some embodiments, the sequences are from Ab0690. In some embodiments, the sequences are from Ab0692. In some embodiments, the sequences are from Ab0698. In some embodiments, the sequences are from Ab0699. In some embodiments, the sequences are from Ab0706. In some embodiments, the sequences are from Ab0707. In some embodiments, the sequences are from Ab0708. In some embodiments, the sequences are from Ab0709. In some embodiments, the sequences are from Ab0710. In some embodiments, the sequences are from Ab0901. In some embodiments, the sequences are from Ab0902. In some embodiments, the sequences are from Ab0903. In some embodiments, the sequences are from Ab0654. In some embodiments, the sequences are from Ab0936. In some embodiments, the sequences are from Ab0937. In some embodiments, the sequences are from Ab0939. In some embodiments, the sequences are from Ab0941. In some embodiments, the sequences are from Ab0944. In some embodiments, the sequences are from Ab0946. In some embodiments, the sequences are from Ab0947. In some embodiments, the sequences are from Ab0953. In some embodiments, the sequences are from Ab0956. In some embodiments, the sequences are from Ab0957. In some embodiments, the sequences are from Ab0958. In some embodiments, the sequences are from Ab1034. In some embodiments, the sequences are from Ab1037. In some embodiments, the sequences are from Ab1044. In some embodiments, the sequences are from Ab1045. In some embodiments, the sequences are from Ab1047. In some embodiments, the sequences are from Ab1051. In some embodiments, the sequences are from Ab1091.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1045 optionally with one or more mutations at positions 50, 58, 95, 98 and 100 in the HC domain. In some embodiments, one or more of the vhCDR1, vhCDR2 and vhCDR3 sequences and the vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1045.

In embodiments, the indinavir-complex binding domain may comprise a VH domain and VL domain selected from the group consisting of the VH and VL domains from FIGS. 6A-6G optionally with one or more mutations. In some embodiments, the indinavir-complex binding domain may comprise a VH domain and VL domain selected from the group consisting of the VH and VL domains from FIGS. 6A-6G.

In some embodiments, the indinavir-complex binding domain may comprise a sequence having at least 85%, 86%, 87%, 88%, 89%, 90%, 91%, 92%, 93%, 94%, 95%, 96%, 97%, 98%, 99%, 100% sequence identity with the VH domain sequence of the Ab1045 and/or a sequence having at least 85%, 86%, 87%, 88%, 89%, 90%, 91%, 92%, 93%, 94%, 95%, 96%, 97%, 98%, 99%, 100% sequence identity with the VL domain sequence of the Ab1045. In some embodiments, the indinavir-complex binding domain may comprise the VH and VL domain sequences of the Ab1045. In some embodiments, the indinavir-complex binding domain may comprise the Ab1045.

In some embodiments, the vhCDR1, vhCDR2 and vhCDR3 sequences and said vlCDR1, vlCDR2, and vlCDR3 sequences may comprise one, two, three, four, five or more mutations selected from the group consisting of mutations corresponding to Y53S, Y53A, Y54S, Y54A, Y58S, Y58A, Y95S, Y95A, Y96S, Y96A, W98S, W98A, Y99S, Y99A, Y100aS, Y100aA, Y100bS, Y100bA, Y100dS, Y100dA, M100eA, M100eL, Y100gS, Y100gA, M100hA, M100hL, F100jA, M100hL, and S30A in the HC domain of Ab0272. In some embodiments, the vhCDR1, vhCDR2 and vhCDR3 sequences and said vlCDR1, vlCDR2, and vlCDR3 sequences may comprise Y96A+Y100gS, Y100bS+Y100gS, Y100dA+Y100gS, Y53A+Y100gS, Y96A+Y100bS+Y100gS, Y53A+Y100bS+Y100gS, Y95A+Y96A, Y100gS+M100hL, or S30A in the HC domain of Ab0272. In some embodiments, the vhCDR1, vhCDR2 and vhCDR3 sequences and said vlCDR1, vlCDR2, and vlCDR3 sequences may comprise one, two, three, four, five or more mutations selected from the group consisting of mutations corresponding to S30A, N28D, N28E, N28T, S30K, V29I, S30K, N28T, V29F, S33A, I24M, H35S, W9Y, W98H, M100eL, and M100hL in the HC domain of Ab0445. In some embodiments, the vhCDR1, vhCDR2 and vhCDR3 sequences and said vlCDR1, vlCDR2, and vlCDR3 sequences may comprise one, two, three, four, five or more mutations selected from the group consisting of mutations corresponding to V29I+S30K, N28T+V29F, N28T+V29F+S33A+I34M+H35S or M100eL+M100hL in the HC domain of Ab0445. In some embodiments, the vhCDR1, vhCDR2 and vhCDR3 sequences and said vlCDR1, vlCDR2, and vlCDR3 sequences may comprise one, two, three or four mutations selected from the group consisting of mutations corresponding to N28D, V29F, M100eL, and M100hL in the HC domain of Ab0596. In some embodiments, the vhCDR1, vhCDR2 and vhCDR3 sequences and said vlCDR1, vlCDR2, and vlCDR3 sequences may comprise one, two, three or four mutations selected from the group consisting of mutations corresponding to V29F+M100eL+M100hL in the HC domain of Ab0596. In some embodiments, the vhCDR1, vhCDR2 and vhCDR3 sequences and said vlCDR1, vlCDR2, and vlCDR3 sequences may comprise one, two, three or four mutations selected from the group consisting of mutations corresponding to M100eA, Y53N, Y53D, Y53H, Y53S, S100bD, S100bK, Y100dN, Y100dD, YS100bK, Y100dN, Y100dD, Y100dH, Y100dK, and Y100dS in the HC domain of Ab00637. In some embodiments, the vhCDR1, vhCDR2 and vhCDR3 sequences and said vlCDR1, vlCDR2, and vlCDR3 sequences may comprise one, two, three or four mutations selected from the group consisting of mutations corresponding to M100eA, Y53H, S100bD, Y53H+S100bD, S100bD+L100eA, and Y53H+S100bD+L100eA in the HC domain of Ab00637. In some embodiments, the vhCDR1, vhCDR2 and vhCDR3 sequences and said vlCDR1, vlCDR2, and vlCDR3 sequences may comprise one, two, three or four mutations selected from the group consisting of mutations corresponding to S50A, I51A, P52aA, S56A, D100bA, G100iA, D101A, G92A, L95A, I96A, and T97A in the HC domain of Ab00698. In some embodiments, the vhCDR1, vhCDR2 and vhCDR3 sequences and said vlCDR1, vlCDR2, and vlCDR3 sequences may comprise one, two or three mutations selected from the group consisting of mutations corresponding to Y58A, Y95A, Y58A+Y95A, Y58A+G100fS, S50A, S50A+W98Y, and S50A+Y58A+Y95A in the HC domain of Ab0902. In some embodiments, the vhCDR1, vhCDR2 and vhCDR3 sequences and said vlCDR1, vlCDR2, and vlCDR3 sequences may comprise a mutation corresponding to Y58A+G100fS and/or S50A in the HC domain of Ab0903. In some embodiments, the vhCDR1, vhCDR2 and vhCDR3 sequences and said vlCDR1, vlCDR2, and vlCDR3 sequences may comprise one or more mutations selected from the group consisting of mutations corresponding to S50A, Y58A, Y95A, and W98Y in the HC domain of Ab0902. In some embodiments, the sequences have a mutation corresponding to said S50A. In some embodiments, the sequences have mutations corresponding to said S50A and said W98Y. In some embodiments, the sequences have mutations corresponding to said S50A, said Y58A, and Y95A.

In some embodiments, the composition of the present disclosure may be a fusion protein.

In some embodiments, the composition may comprise another VH domain and another VL domain. In some embodiments, the composition may comprise two identical VH domains and two identical VL domains.

In some embodiments, the composition may comprise an indinavir-complex binding domain comprising scFv means for binding a complex of indinavir bound to a scFv. In some embodiments, the composition may comprise an indinavir-complex binding domain comprising Fab means for binding indinavir complex with an indinavir binding domain.

In some embodiments, said composition comprises an Fc domain with one or more knob variants. In some embodiments, said composition comprises an Fc domain with one or more hole variants. Some examples of the knob and hole Fc sequences are disclosed in FIG. 10. In some embodiments, the Fc domain described herein having a knob variant comprises a sequence having at least 85, 86, 87, 88, 89, 90, 91, 92, 93, 94, 95, 96, 97, 98, 99 and 100% identity to any one of SEQ ID NO: 3235, 3237, 3239, 3241, and 3243. In some embodiments, the Fc domain described herein having a hole variant comprises a sequence having at least 85, 86, 87, 88, 89, 90, 91, 92, 93, 94, 95, 96, 97, 98, 99 and 100% identity to any one of SEQ ID NO: 3236, 3238, 3240, 3242, and 3244. In some embodiments, the Fc domain described herein having a knob variant comprises the sequence of 3235. In some embodiments, the Fc domain described herein having a knob variant comprises the sequence of 3237. In some embodiments, the Fc domain described herein having a knob variant comprises the sequence of 3239. In some embodiments, the Fc domain described herein having a knob variant comprises the sequence of 3241. In some embodiments, the Fc domain described herein having a knob variant comprises the sequence of 3243. In some embodiments, the Fc domain described herein having a hole variant comprises the sequence of SEQ ID NO: 3236. In some embodiments, the Fc domain described herein having a hole variant comprises the sequence of SEQ ID NO: 3238. In some embodiments, the Fc domain described herein having a hole variant comprises the sequence of SEQ ID NO: 3240. In some embodiments, the Fc domain described herein having a hole variant comprises the sequence of SEQ ID NO: 3242. In some embodiments, the Fc domain described herein having a hole variant comprises the sequence of SEQ ID NO: 3244.

In some embodiments, the composition may be a nucleic acid composition comprising a polynucleotide encoding a composition comprising an indinavir binding domain. In some embodiments, an expression vector composition may comprise the nucleic acid composition. In some embodiments, a host cell may comprise the expression vector.

In some embodiments, the method of making the composition of the present disclosure may comprise an immunization campaign, validation of murine indinavir binders, humanization of the murine indinavir binders, generation of the composition and validation of the composition.

For example, in some embodiments, the first CID domain is an indinavir binding domain and the CID small molecule is indinavir. The second CID domain comprises a heavy chain variable domain and light chain variable domain comprising the amino acid sequences of vhCDRs and vlCDRs as shown in FIGS. 6A-6G. The second CID domain binds specifically to the complex formed between the first CID domain and the CID small molecule, but may not bind or only weakly bind to the first CID domain without the CID small molecule and may not bind or only weakly bind to the free CID small molecule.

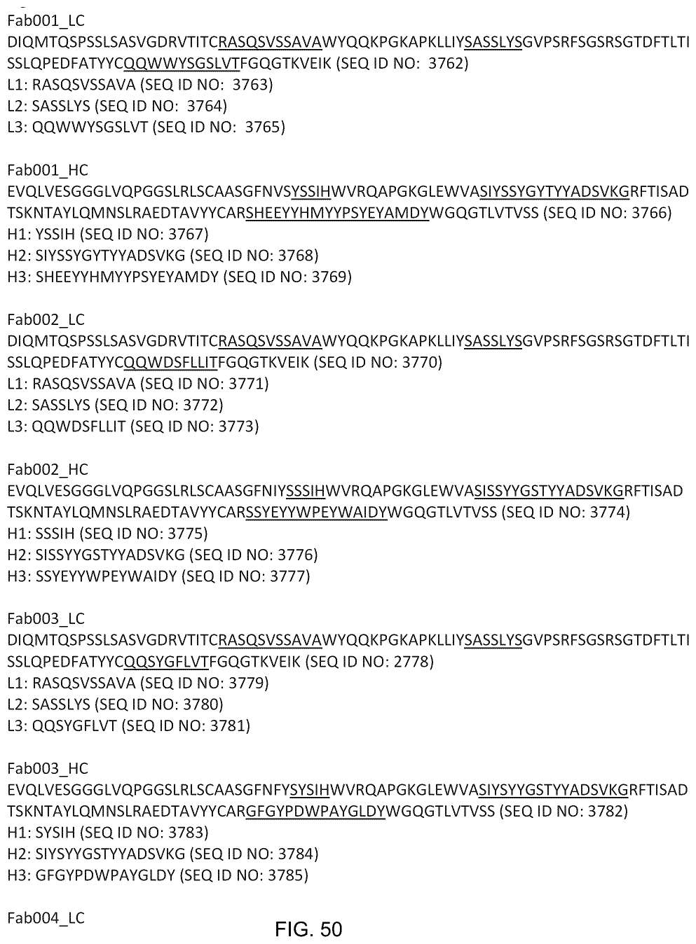

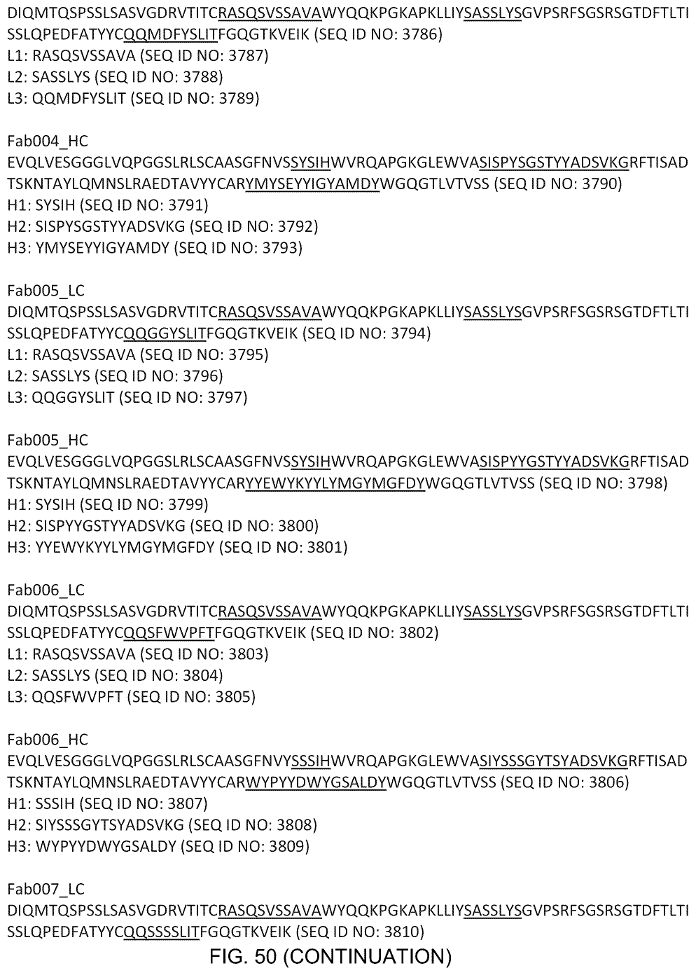

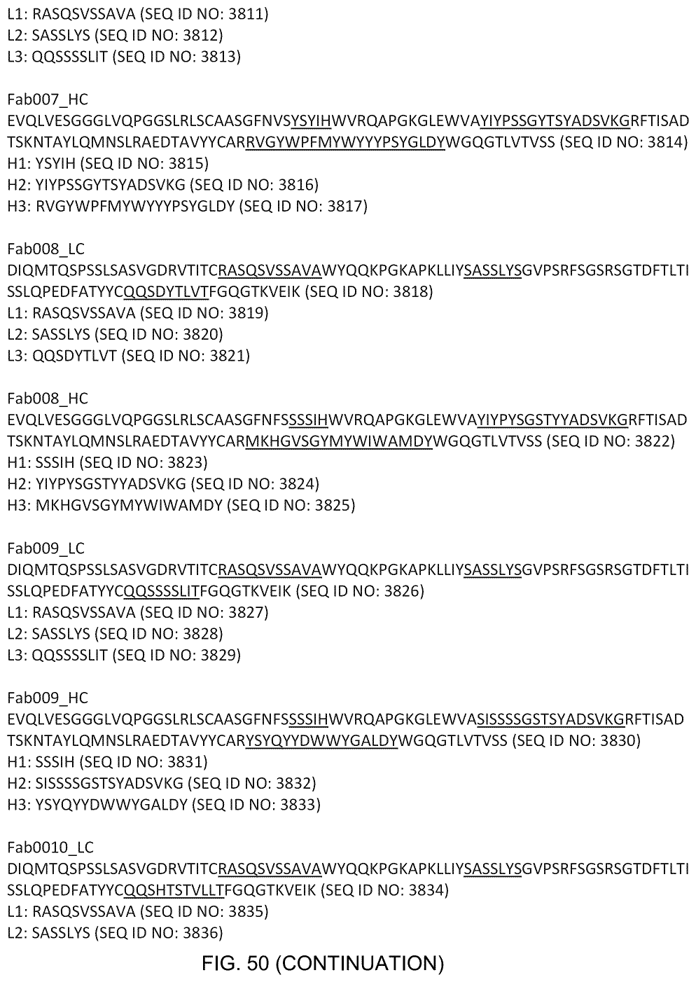

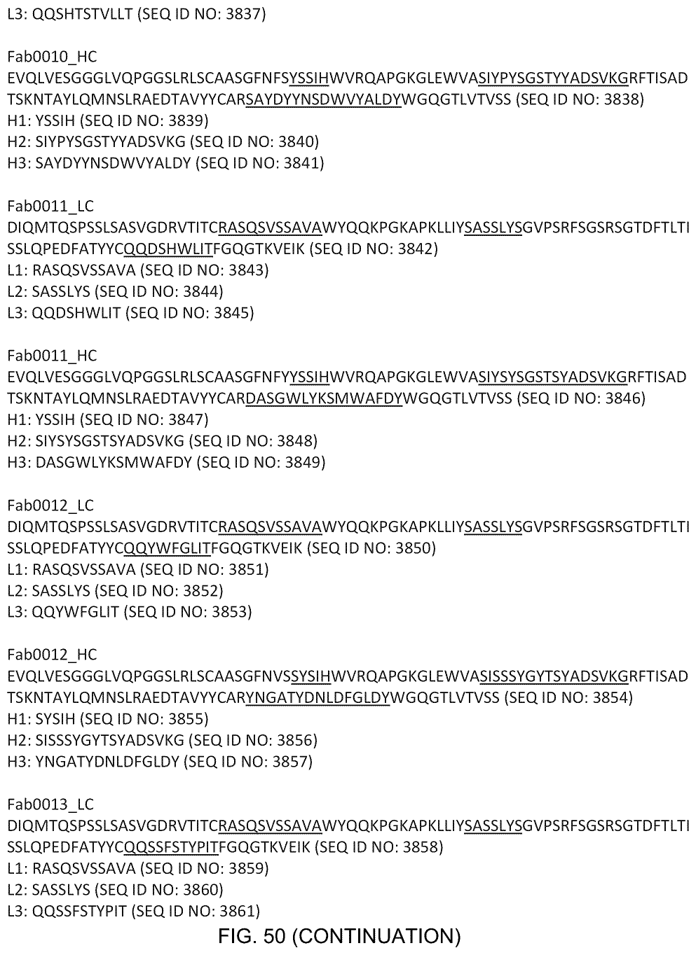

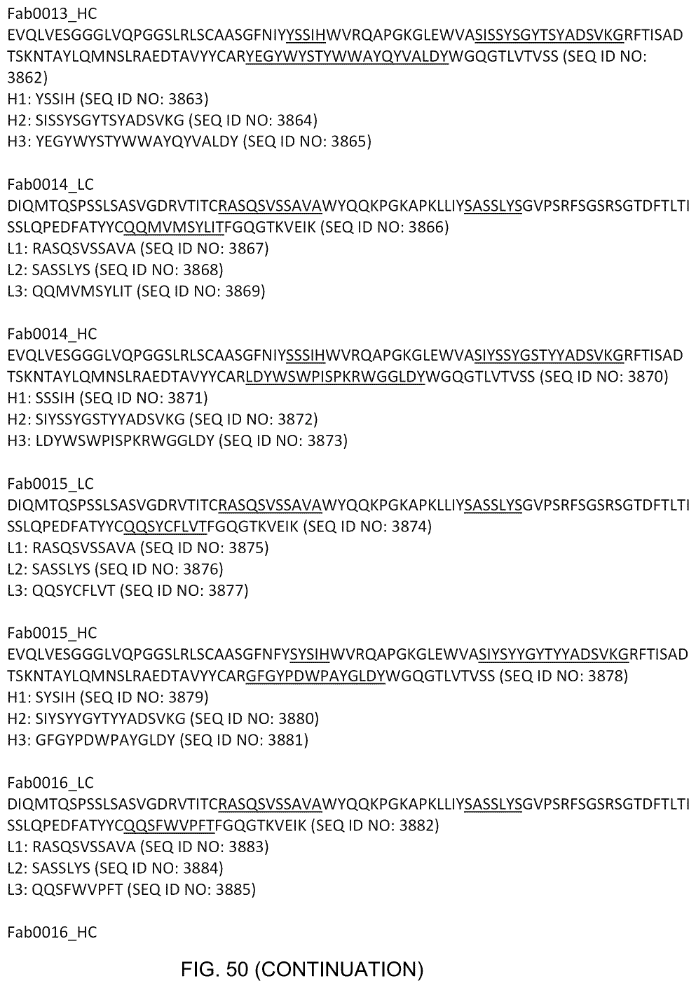

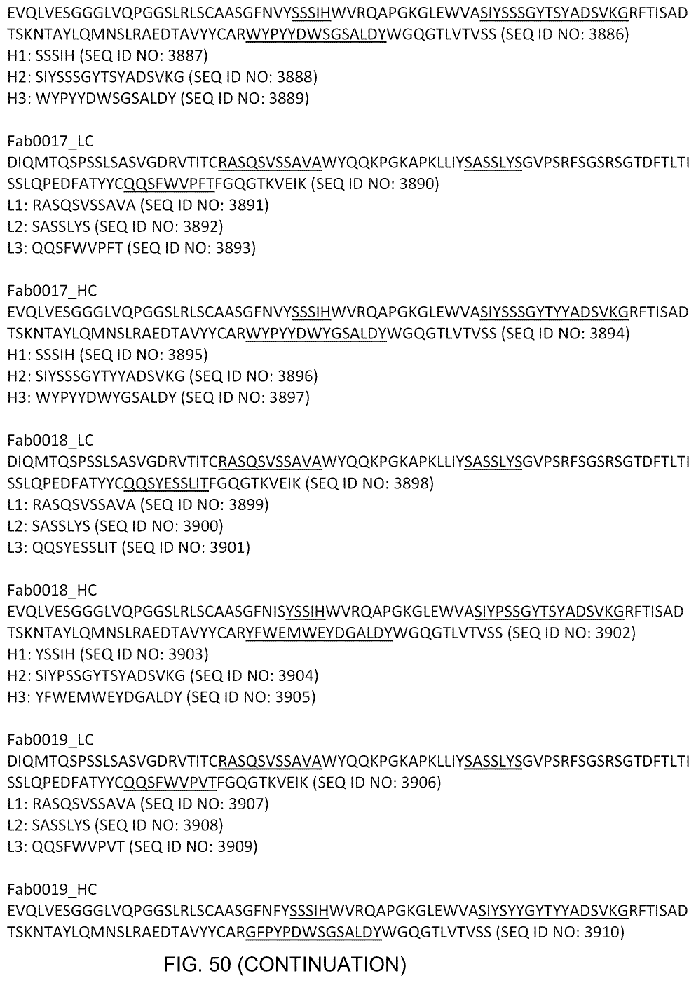

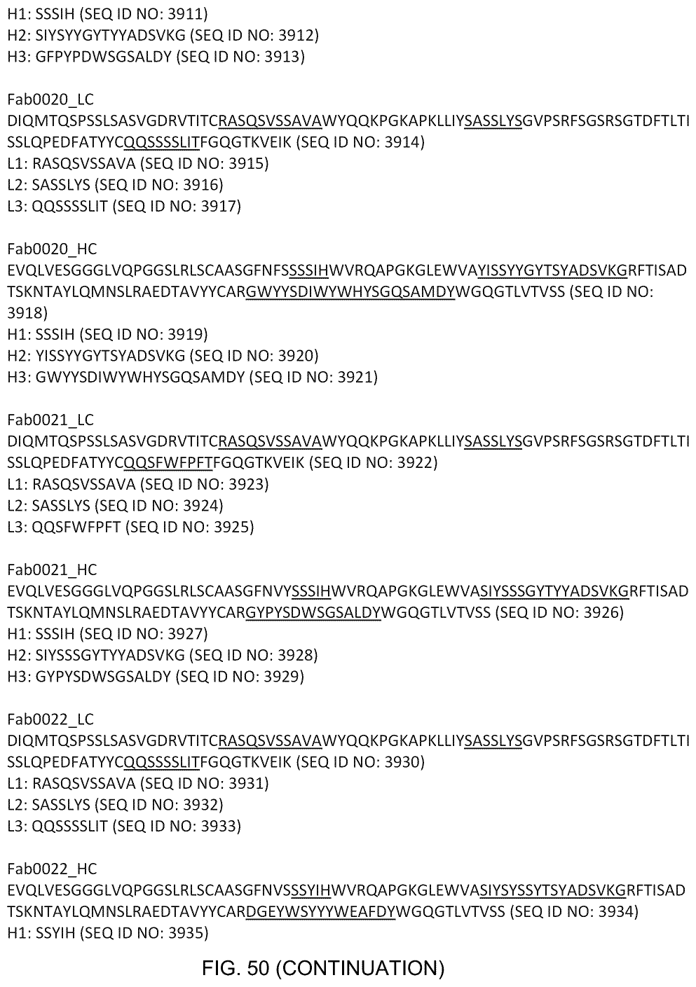

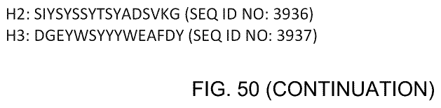

In another aspect, the present disclosure relates to a composition comprising an indinavir-complex binding domain comprising: a) a variable heavy (VH) domain comprising a vhCDR1, vhCDR2, and vhCDR3 sequence; b) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). In another aspect, the present disclosure relates to a composition according to claim B1 wherein said indinavir-complex binding domain comprises a VH domain and VL domain selected from the group consisting of the VH and VL domains of Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). In some embodiments, said composition is a fusion protein. In some embodiments, the composition comprises an indinavir-complex binding domain comprising scFv means for binding a complex of indinavir bound to a scFv. In some embodiments, the composition comprises an indinavir binding domain comprising Fab means for binding indinavir. In some embodiments, the present disclosure relates to a nucleic acid composition comprising a polynucleotide encoding said composition. In some embodiments, the present disclosure relates to an expression vector composition comprising the nucleic acid composition. In some embodiments, the present disclosure relates to a host cell comprising the expression vector composition. In some embodiments, the present disclosure relates to a method of making the composition, the method comprising: an immunization campaign, validation of murine indinavir binders, humanization of the murine indinavir binders, generation of the composition, and validation of the composition.

In one aspect, the disclosure provides a composition comprising a first protein having the composition comprising an indinavir binding domain as disclosed herein and a second protein having the composition comprising an indinavir-complex binding domain as disclosed herein.

In another aspect, the present disclosure relates to a composition comprising: a) a first protein comprising an indinavir binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4- D11, P7-D12, P1-C11, P4-F9, P6-HS, P2-D7, P3-FS, P3-C5, P8-F4, P8-C2, P7-F12, P2-H7, P1-B7, P2- D9, P6-E8, P3-B9, P1-G1, P1-D11, P8-E3, P4-E4, PS-D12, P3-F4, P6-H2, P1-E9, P8-D9, and P4-G11 (FIG. 49); and b) a second protein comprising an indinavir-complex binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). In some embodiments, said indinavir binding domain comprises a VH domain and VL domain selected from the group consisting of the VH and VL domains of P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4-D11, P7-D12, P1-C11, P4-F9, P6-HS, P2-D7, P3-FS, P3-C5, P8-F4, P8-C2, P7-F12, P2-H7, P1-B7, P2-D9, P6-E8, P3-B9, P1-G1, P1-D11, P8-E3, P4-E4, PS-D12, P3-F4, P6-H2, P1-E9, P8-D9, and P4- G11 (Figure X) and said indinavir-complex binding domain comprises a VH domain and VL domain selected from the group consisting of the VH and VL domains of Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). In some embodiments, the indinavir binding domain comprises the VH and VL domain of PS-HS. In some embodiments, the indinavir-complex binding domain comprises the VH and VL domain of Fab005.

In some embodiments, the CID small molecule is indinavir, and the first iCID domain is an indinavir ABD which comprises a heavy chain variable domain and light chain variable domain comprising the amino acid sequences of vh-CDR1, vh-CDR2, vh-CDR3, vl-CDR1, vl-CDR2, and vl-CDR3, respectively, as shown in FIGS. 5A-5E. The second iCID domain comprises an ABD capable of specifically binding to the complex between indinavir and the first iCID domain, and the second iCID domain comprises vhCDRs and vlCDRs as shown in FIGS. 6A-6G.

In some embodiments, the second half of the iCID comprises an ABD and binds to a site of the complex comprising at least a portion of the small molecule and a portion of the first half of the iCID. In some embodiments, the second half of the iCID comprises an ABD, and binds to a site of the complex of the small molecule and the first half of the iCID, wherein the second half of the iCID binds to the site comprising at least one atom of the small molecule and one atom of the first half of the iCID.

In some embodiments, the second half of the iCID binds to the complex of the first half of the iCID and the small molecule with a dissociation constant (K_{D}) no more than about 1/250 times (such as no more than about any of 1/300, 1/350, 1/400, 1/450, 1/500, 1/600, 1/700, 1/800, 1/900, 1/1000, 1/1 100, 1/1200, 1/1300, 1/1400, or 1/1500 times, or less) its K_{D} for binding to each of the free small molecule and the free first half of the iCID.

Binding moieties that specifically bind to a complex between a small molecule and a cognate binding moiety can be produced according to methods known in the art, see, for example, WO2018/213848, hereby incorporated herein by reference in its entirety and specifically for the methods for producing iCID domains. Briefly, a screening is performed from an antibody library, a DARPin library, a nanobody library, or an aptamer library or a phage displayed Fab library. For example, as the step 1, binding moieties can be selected that do not bind to the cognate binding moiety in the absence of the small molecule, thereby generating a set of counter selected binding moieties; and then, as step 2, the counter selected binding moieties can be screened for binding moieties that bind to the complex of the small molecule and the cognate binding moiety, thereby generating a set of positively selected binding moieties. Steps 1 and 2 of screening can be conducted one or more rounds, wherein each round of screening comprises the screening of step 1 and the screening of step 2, such that a set of binding moieties that specifically bind to the complex between the small molecule and the cognate binding moiety is generated. In some embodiments, two or more rounds of screening are performed, wherein the input set of binding moieties of step 1 for the first round of screening is the binding molecule library; the input set of binding moieties of step 2 for each round of screening is the set of counter selected binding moieties of step 1 from the given round of screening; the input set of binding moieties of step 1 for each round of screening following the first round of screening is the set of positively selected binding moieties of step 2 from the previous round of screening; and the set of binding moieties that specifically bind to the complex between the small molecule and the cognate binding moiety is the set of positively selected binding moieties of step 2 for the last round of screening.

Phage display screening can be done according to previously established protocols (see, Seiler, et al, Nucleic Acids Res., 42:D12531260 (2014). For example, to select antibody binding moieties for the complex of BCL-xL and ABT-737 or, as described herein, for the complex of iCID and indinavir, antibody phage library can be screen against biotinylated iCID captured with streptavidincoated magnetic beads (Promega). Prior to each selection, the phage pool can be incubated with 1 mM of iCID immobilized on streptavidin beads in the absence of indinavir in order to deplete the library of any binders to the apo form of iCID. Subsequently, the beads can be removed and indinavir can be added to the phage pool at a concentration of 1 mM. In total, four rounds of selection can be performed with decreasing amounts of iCID antigen (100 nM, 50 nM, 10 nM and 10 nM). To reduce the deleterious effects of nonspecific binding phage, specific iCID binding Fab-phage can be selectively eluted from the magnetic beads by addition of 2 g/mL TEV protease. Individual phage clones from the fourth round of selection can then be analyzed for sequencing.

### 2. Fc Domains

In addition to the iCID domains, the pairs of fusion polypeptides that make up the formats of the invention generally comprise an Fc domain.

As will be appreciated by those in the art, there are generally three types of Fc domains that find use in various embodiments of the present invention, including heterodimeric Fc domains, homodimeric Fc domains and monomeric Fc domains. Additionally, as fully described below, the CC and CT proteins can incorporate any one of the three types of Fc domains, and these can be additionally mixed and matched in the protein complexes. As will be appreciated by those in the art, Fc domains derived from human IgG1 or IgG2, for example, will self-assemble to form dimers (either homodimers or heterodimers as discussed herein), while Fc domains derived from an IgG4 Fc domain are monomeric, and won't self-assemble.

In addition to the iCID domains, the CC and CTCoS fusion polypeptides generally comprise an Fc domain. As will be appreciated by those in the art, there are generally three types of Fc domains that find use in various embodiments of the present invention, including heterodimeric Fc domains, homodimeric Fc domains and monomeric Fc domains. Additionally, as fully described below, the CC and CTCoS proteins can incorporate any one of the three types of Fc domains, and these can be additionally mixed and matched in the protein complexes. As will be appreciated by those in the art, Fc domains derived from human IgG1 or IgG2, for example, will self-assemble to form dimers (either homodimers or heterodimers as discussed herein), while Fc domains derived from an IgG4 Fc domain are monomeric, and won't self-assemble.

In addition to the iCID domains, the CC and CTTCoS fusion polypeptides generally comprise an Fc domain. As will be appreciated by those in the art, there are generally three types of Fc domains that find use in various embodiments of the present invention, including heterodimeric Fc domains, homodimeric Fc domains and monomeric Fc domains. Additionally, as fully described below, the CC and CTTCoS proteins can incorporate any one of the three types of Fc domains, and these can be additionally mixed and matched in the protein complexes. As will be appreciated by those in the art, Fc domains derived from human IgG1 or IgG2, for example, will self-assemble to form dimers (either homodimers or heterodimers as discussed herein), while Fc domains derived from an IgG4 Fc domain are monomeric, and won't self-assemble.

In some embodiments, the Fc domain used has the formula (N- to C-terminal) hinge-CH2-CH3, wherein the hinge is either a full or partial hinge sequence. In some embodiments, the Fc domain used has the formula (N- to C-terminal) CH2-CH3. In some cases, as discussed below, domain linkers can be used to link the Fc domain to the other components.

a. Heterodimeric Fc Variant Domains

As discussed herein, some embodiments of the invention utilize a CC and CT binding protein that each contains one of a pair of heterodimeric Fc domains. Accordingly, in some embodiments, the invention provides heterodimeric Fc variant domains which include modifications that facilitate the heterodimerization of two Fc domains and/or allow for ease of purification of heterodimers over homodimers, collectively referred to herein as “heterodimerization variants.” As is known in the art, there are a number of mechanisms that can be used to generate heterodimeric Fc domains. Amino acid variants that lead to the production of heterodimeric Fc domains are referred to as “heterodimerization variants”. As discussed below, heterodimerization variants can include steric variants (e.g. the “knobs and holes” variants and the “charge pairs” variants described below) that “skew” the formation of A-B Fc heterodimers over A-A and B-B Fc homodimers. Some examples of the knob and hole Fc sequences described above.

As discussed herein, some embodiments of the invention utilize a CC and CTCoS binding protein that each contains one of a pair of heterodimeric Fc domains. Accordingly, in some embodiments, the invention provides heterodimeric Fc variant domains which include modifications that facilitate the heterodimerization of two Fc domains and/or allow for ease of purification of heterodimers over homodimers, collectively referred to herein as “heterodimerization variants.” As is known in the art, there are a number of mechanisms that can be used to generate heterodimeric Fc domains. Amino acid variants that lead to the production of heterodimeric Fc domains are referred to as “heterodimerization variants”. As discussed below, heterodimerization variants can include steric variants (e.g. the “knobs and holes” variants and the “charge pairs” variants described below) that “skew” the formation of A-B Fc heterodimers over A-A and B-B Fc homodimers. Some examples of the knob and hole Fc sequences described above.

As discussed herein, some embodiments of the invention utilize a CC and CTTCoS binding protein that each contains one of a pair of heterodimeric Fc domains. Accordingly, in some embodiments, the invention provides heterodimeric Fc variant domains which include modifications that facilitate the heterodimerization of two Fc domains and/or allow for ease of purification of heterodimers over homodimers, collectively referred to herein as “heterodimerization variants.” As is known in the art, there are a number of mechanisms that can be used to generate heterodimeric Fc domains. Amino acid variants that lead to the production of heterodimeric Fc domains are referred to as “heterodimerization variants”. As discussed below, heterodimerization variants can include steric variants (e.g. the “knobs and holes” variants and the “charge pairs” variants described below) that “skew” the formation of A-B Fc heterodimers over A-A and B-B Fc homodimers.

One mechanism is generally referred to in the art as “knobs and holes”, or KIH referring to amino acid engineering that creates steric influences to favor heterodimeric formation and disfavor homodimeric formation. That is, one monomer is engineered to have a bulky amino acid (a “knob”) and the other is engineered to reduce the size of the amino acid side chain (a “hole”), that skews the formation of heterodimers over homodimers. These techniques and sequences are described in Ridgway et al., Protein Engineering 9(7):617 (1996); Atwell et al., J. Mol. Biol. 1997 270:26; U.S. Pat. No. 8,216,805, US 2012/0149876, all of which are hereby incorporated by reference in their entirety. The Figures of these references (also specifically incorporated by reference herein for the amino acid variants) identify a number of “monomer A-monomer B” pairs that rely on “knobs and holes”. In addition, as described in Merchant et al., Nature Biotech. 16:677 (1998), these “knobs and hole” mutations can be combined with disulfide bonds to skew formation to heterodimerization.

An additional mechanism that finds use in the generation of heterodimers is sometimes referred to as “electrostatic steering” or “charge pairs” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants”. These include, but are not limited to, D221E/P228E/L368E paired with D221R/P228R/K409R (e.g. these are “monomer corresponding sets) and C220E/P228E/368E paired with C220R/E224R/P228R/K409R.

Exemplary methods for introducing heterodimerization variants include symmetric-to-asymmetric steric complementarity design, e.g., introducing KiH, HA-TF, and ZW1 mutations [see, Atwell et al., J Mol Biol (1997) 270(1):26-35; Moore et al., MAbs (2011) 3(6):546-57; Von Kreudenstein et al., MAbs (2013) 5(5):646-54, all of which are expressly incorporated herein by reference in their entirety]; charge-to-charge swap (e.g., introducing DD-KK mutations)(see, Gunasekaran et al., J Biol Chem 2010; 285:19637-46 incorporated herein by reference in its entirety); charge-to-steric complementarity swap plus additional long-range electrostatic interactions (e.g., introducing EW-RVT mutations) (Choi et al., Mol Cancer Ther (2013) 12(12):2748-59 incorporated herein by reference in its entirety); and isotype strand swap, e.g., introducing strand-exchange engineered domain (SEED) (Klein et al, MAbs (2012) 4(6):653-63; Von Kreudenstein et al., MAbs (2013) 5(5):646-54, all of which are expressly incorporated herein by reference in their entirety). Exemplary mutations which can be introduced into the Fc domains for inducing heterodimerization are summarized in Table 2.

TABLE 2

| Heterodimeric | Paired mutation - | Paired mutation - |
|---|---|---|
| Fc domain name | one Fc domain | cognate Fc domain |
| KiH | T366W | T366S/L368A/Y407V |
| KiHS-S | T366W/S354C | T366S/L368A/Y407V/ |
| | | Y349C |
| HA-TF | S364H/F405A | Y349T/T394F |
| ZW1 | T350V/L351Y/F405A/ | T350V/T366L/K392L/ |
| | Y407V | T394W |
| 7.8.60 | K360D/D399M/ | E345R/Q347R/T366V/ |
| | Y407A | K409V |
| DD-KK | K409D/K392D | D399K/E356K |
| EW-RVT | K360E/K409W | Q347R/D399V/F405T |
| EW-RVTS-S | K360E/K409W/ | Q347R/D399V/F405T/ |
| | Y349C | S354C |
| SEED | IgA-derived 45 residues | IgG1-derived 57 residues |
| | on IgG1 CH3 | on IgA CH3 |
| A107 | K370E/K409W | E357N/D399V/F405T |

In some embodiments, KIH mutations are introduced in the Fc domains of IgG1, IgG2, IgG3 or IgG4. Additional exemplary KIH mutations are listed in Table 3, and can be found in U.S. Pat. No. 8,216,805, which is incorporated by reference in its entirety

TABLE 3

| Paired mutation - | Paired mutation - |
|---|---|
| one Fc domain | cognate Fc domain |
| T366Y | Y407T |
| T366W | Y407A |
| F405A | T394W |
| Y407T | T366Y |
| T366Y/F405A | T394W/Y407T |
| T366W/F405W | T394S/Y407A |
| F405W/Y407A | T366W/T394S |
| F405W | T394S |

In addition to the amino acid substitutions that confer heterodimerization, additional amino acid variations can be introduced into the Fc domain for additional functionality like altered or ablated binding to Fcγ and FcRn receptors, etc., which are described in detail herein.

b. Homodimeric Fc Domains

Alternatively, some formats of the invention rely on the inclusion of Fc domains that self-assemble to form homodimeric Fc domains. In some embodiments, the Fc domains used for forming a homodimeric Fc fusion protein can be derived from the Fc domains of an IgG, including an IgG1, IgG2, IgG3 or IgG4.

In some embodiments, the Fc domains are derived from an IgG1, IgG2, IgG3 or IgG4 Fc domain which includes a hinge or partial hinge, a CH2 domain, a CH3 domain. the Fc domains are derived from an IgG1, IgG2, IgG3 or IgG4 Fc domain which includes a CH2 domain and a CH3 domain without a hinge.

In some embodiments, the amino acid sequence of homodimeric Fc domains is at least 80%, 85%, 90%, or 95% identical to a human IgG1, IgG2, IgG3, or IgG4 Fc domain with or without the hinge.

The homodimeric Fc domains may also include modifications that affect functionality including, but not limited to, altering binding to one or more Fc receptors (e.g., FcγR and FcRn) as described herein.

In some embodiments, a human IgG1 Fc domain or variants therein find use in this invention (e.g. IgG1 Fc, see FIG. 10).

In some embodiments, the Fc domain described herein having a knob variant comprises a sequence having at least 85, 86, 87, 88, 89, 90, 91, 92, 93, 94, 95, 96, 97, 98, 99 and 100% identity to any one of SEQ ID NO: 3235, 3237, 3239, 3241, and 3243. In some embodiments, the Fc domain described herein having a hole variant comprises a sequence having at least 85, 86, 87, 88, 89, 90, 91, 92, 93, 94, 95, 96, 97, 98, 99 and 100% identity to any one of SEQ ID NO: 3236, 3238, 3240, 3242, and 3244. In some embodiments, the Fc domain described herein having a knob variant comprises the sequence of 3235. In some embodiments, the Fc domain described herein having a knob variant comprises the sequence of 3237. In some embodiments, the Fc domain described herein having a knob variant comprises the sequence of 3239. In some embodiments, the Fc domain described herein having a knob variant comprises the sequence of 3241. In some embodiments, the Fc domain described herein having a knob variant comprises the sequence of 3243. In some embodiments, the Fc domain described herein having a hole variant comprises the sequence of SEQ ID NO: 3236. In some embodiments, the Fc domain described herein having a hole variant comprises the sequence of SEQ ID NO: 3238. In some embodiments, the Fc domain described herein having a hole variant comprises the sequence of SEQ ID NO: 3240. In some embodiments, the Fc domain described herein having a hole variant comprises the sequence of SEQ ID NO: 3242. In some embodiments, the Fc domain described herein having a hole variant comprises the sequence of SEQ ID NO: 3244.

c. Monomeric IgG4 Fc Domains

In some embodiments, the CC and/or CT binding proteins comprises a variant IgG4 Fc domain which inhibits dimer formation of the Fc domains.

In some embodiments, the CC and/or CTCoS binding proteins comprises a variant IgG4 Fc domain which inhibits dimer formation of the Fc domains.

In some embodiments, the CC and/or CTTCoS binding proteins comprises a variant IgG4 Fc domain which inhibits dimer formation of the Fc domains.

One or more amino acid substitutions can be introduced into the Fc domain of human IgG4 to inhibit dimer formation of the Fc domain. These substitutions can be at one or more of the following amino acids according to the Kabat EU numbering system: 349, 351, 354, 356, 357, 364, 366, 368, 370, 392, 394, 399, 405, 407,409, 409 and 439. In some embodiments, the IgG4 Fc domain comprises one or more of the following amino acid substitutions: L351R, L351D, E357R, E357W, S364R, T366R, L368R, T394R, T394D, D399R, F405R, F405Q, Y407R, Y407D, K409W and R409W. In some other embodiments, the IgG4 Fc domain comprises one or more of the following sets of amino acid substitutions: Y349D/S354D, L351DMT394D, L351D/K409R, L351RMT394R, E356R/D399R, D356R/D399R, S364R/L368R, S364W/L368W, S364W/K409R, T366R/Y407R, T366W/L368W, L368R/K409R, T394D/K409R, D399R/K409R, D399R/K439D, F405A/Y407A, F405Q/Y407Q, L351RMT364RMT394R and T366Q/F405Q/Y407Q. In some embodiments, the IgG4 Fc domain comprises L351F, T366R, P395K, F405R and Y407E. In some embodiment, the monomeric IgG4 Fc domain is hingeless, and comprises one or more of the following sets of amino acid substitutions: L351D, L351R, S364R, T366R, L368R, T394D, D399R, F405Q, F405R, Y407R, L351DMT394D, L351DMT394R, S364R/L368R, S364W/L368W, T366R/Y407R, and T366W/L368W. More mutations that stabilize the monomeric IgG4 Fc domain can be found in US Patent Application Publication No. 20130177555, Wilkinson et al., MAbs (2013) 5:606-17, and Shan et al., PLOS ONE|DOI:10.1371/journal.pone.0160345 Aug. 1, 2016, all of which are expressly incorporated herein by reference in their entirety.

In some embodiments, the monomeric IgG4 Fc domain used in the present invention has the amino acid sequence of IgG4 monomeric Fc, see FIG. 10.

In some embodiments, the Fc domain described herein having a knob variant comprises a sequence having at least 85, 86, 87, 88, 89, 90, 91, 92, 93, 94, 95, 96, 97, 98, 99 and 100% identity to any one of SEQ ID NO: 3235, 3237, 3239, 3241, and 3243. In some embodiments, the Fc domain described herein having a hole variant comprises a sequence having at least 85, 86, 87, 88, 89, 90, 91, 92, 93, 94, 95, 96, 97, 98, 99 and 100% identity to any one of SEQ ID NO: 3236, 3238, 3240, 3242, and 3244. In some embodiments, the Fc domain described herein having a knob variant comprises the sequence of 3235. In some embodiments, the Fc domain described herein having a knob variant comprises the sequence of 3237. In some embodiments, the Fc domain described herein having a knob variant comprises the sequence of 3239. In some embodiments, the Fc domain described herein having a knob variant comprises the sequence of 3241. In some embodiments, the Fc domain described herein having a knob variant comprises the sequence of 3243. In some embodiments, the Fc domain described herein having a hole variant comprises the sequence of SEQ ID NO: 3236. In some embodiments, the Fc domain described herein having a hole variant comprises the sequence of SEQ ID NO: 3238. In some embodiments, the Fc domain described herein having a hole variant comprises the sequence of SEQ ID NO: 3240. In some embodiments, the Fc domain described herein having a hole variant comprises the sequence of SEQ ID NO: 3242. In some embodiments, the Fc domain described herein having a hole variant comprises the sequence of SEQ ID NO: 3244.

As for the other Fc domains, monomeric Fc domains can also include additional variants for functional alterations.

d. Fc Domain Variants

Fc domains used herein may independently include Fc modifications that affect functionality including, but not limited to, altering binding to one or more Fc receptors (e.g., FcγR and FcRn).

### (i) FcγR Variants

In some embodiments, Fc domains used herein include one or more amino acid modifications that affect binding to one or more Fcγ receptors (e.g., “FcγR variants”). FcγR variants (e.g., amino acid substitutions) that result in increased binding as well as decreased binding can be useful. For example, it is known that increased binding to FcγRIIIa results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. FcγR variants that reduce FcγR activation and Fc-mediated toxicity such as P329G, L234A, L235A can find use in the Fc fusion proteins in the current invention (see, Schlothauer et al. Protein Eng Des Sel. 2016; 29(10):457-466 incorporated herein for reference in its entirety). For example, IgG1 Fc domain incorporating P329G, L234A, L235A can be used in the current invention, and can be further modified to facilitate heterodimerization.

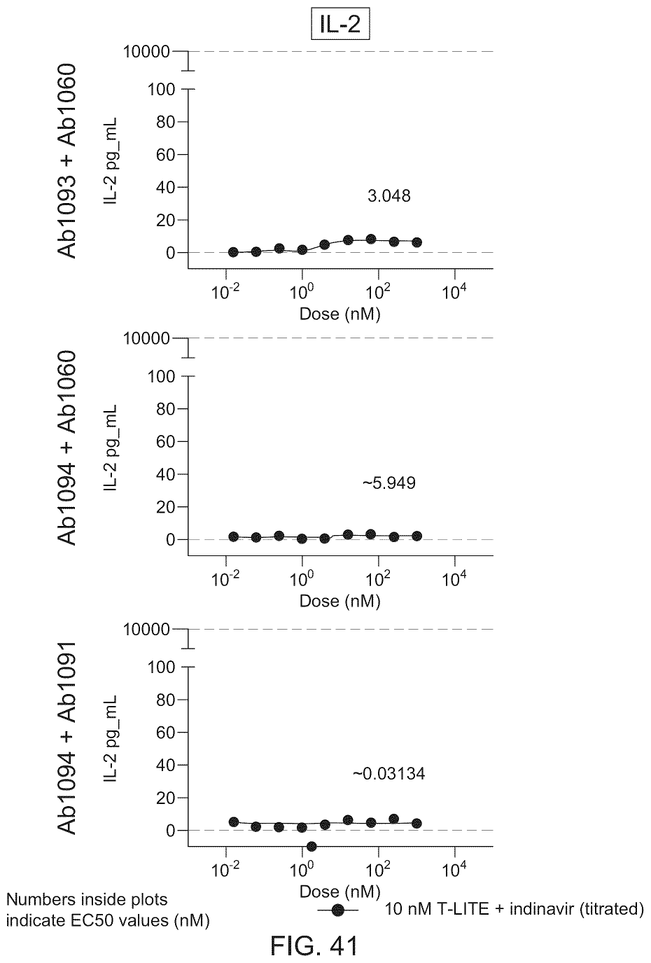

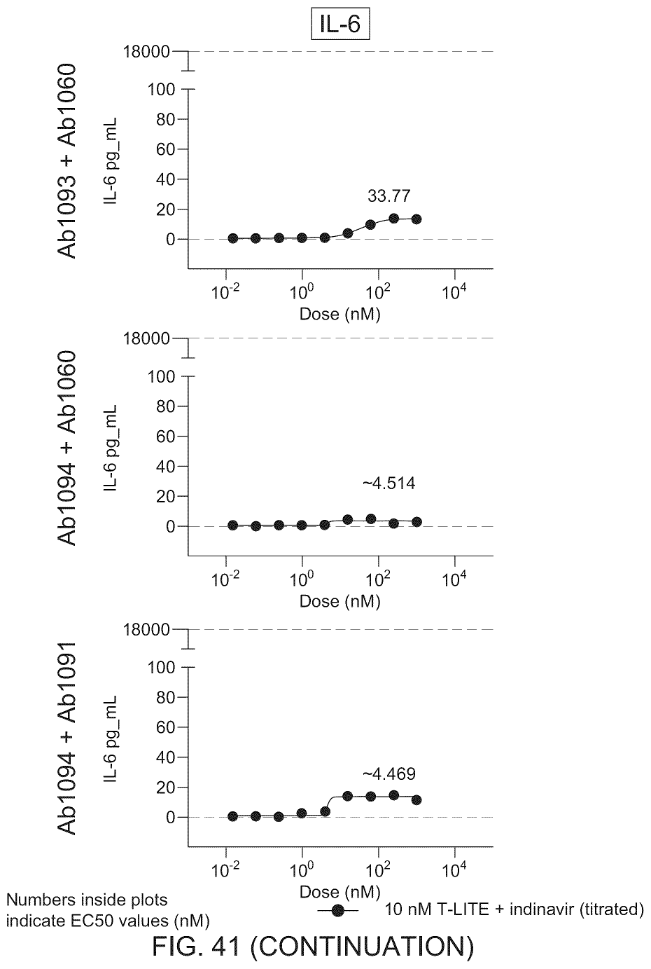

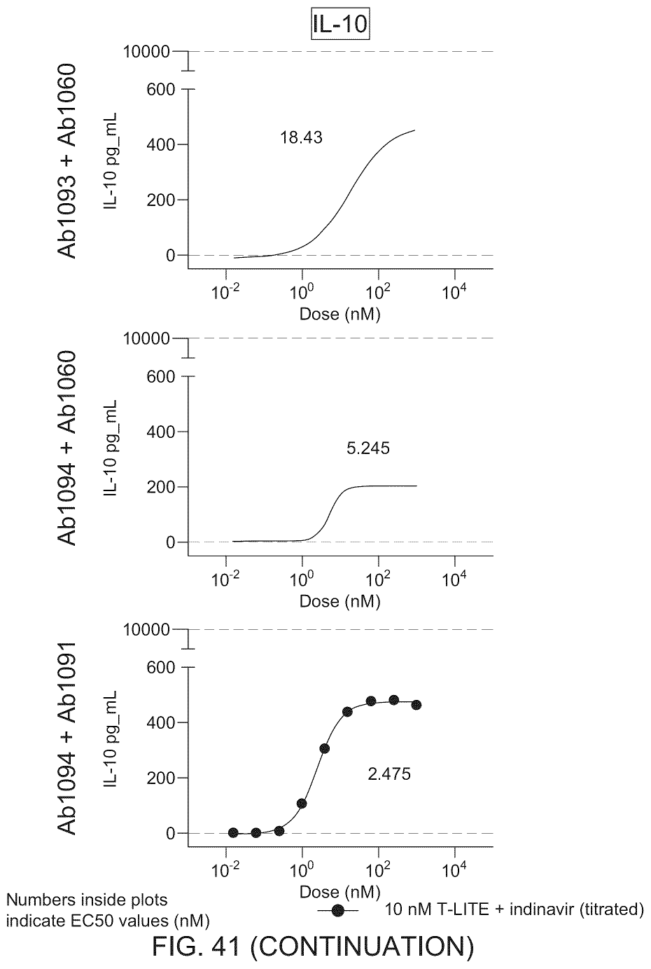

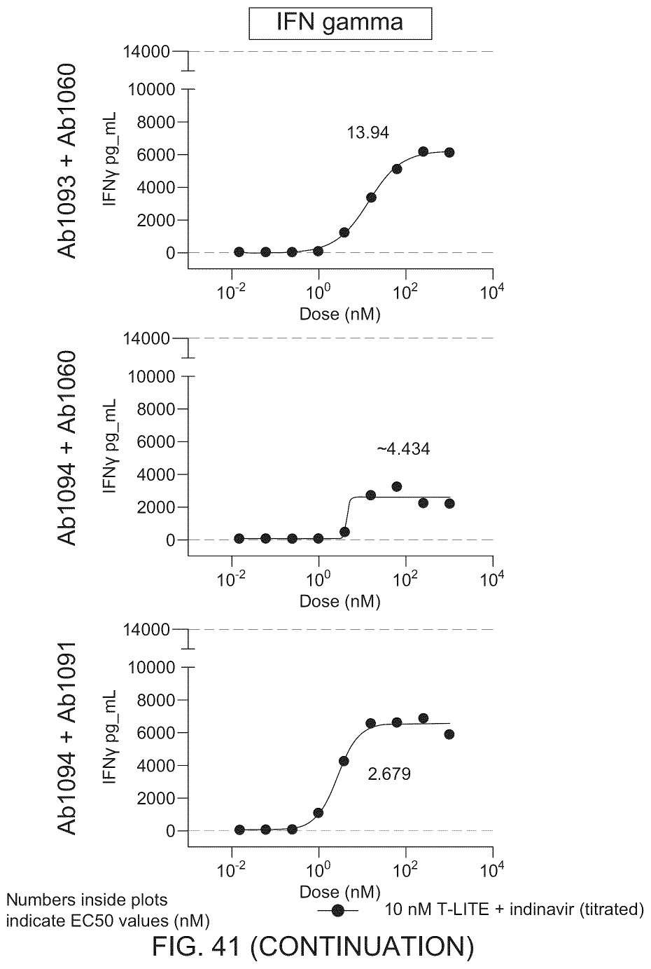

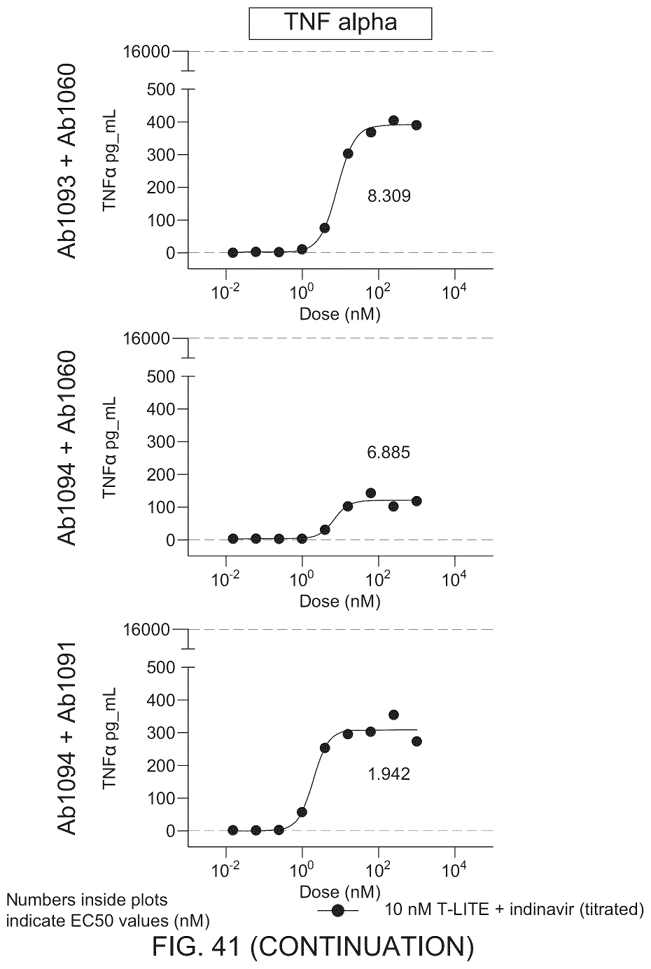

Additional FcγR variants can include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T.

### (ii) FcRn Variants

Further, Fc domains used herein can independently include Fc substitutions that confer increased binding to the FcRn and increased serum half-life. Such modifications are disclosed, for example, in U.S. Pat. No. 8,367,805, hereby incorporated by reference in its entirety, and specifically for Fc substitutions that increase binding to FcRn and increase half-life. Such modifications include, but are not limited to 434S, 434A, 428L, 308F, 259I, 428L/434S, 259I/308F, 436I/428L, 436I or V/434S, 436V/428L and 259I/308F/428L.

(iii) Ablation Variants

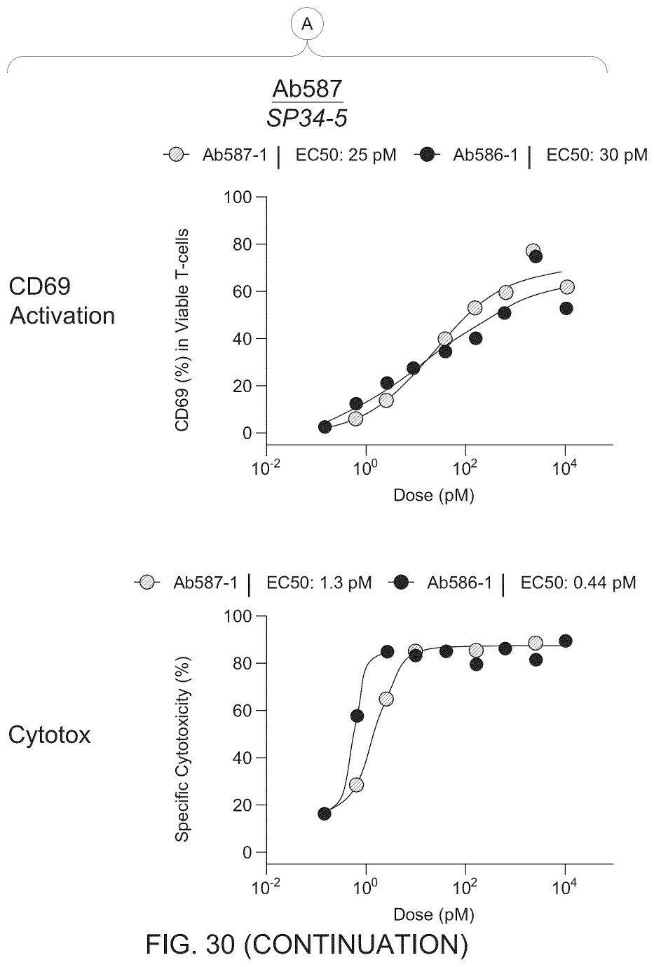

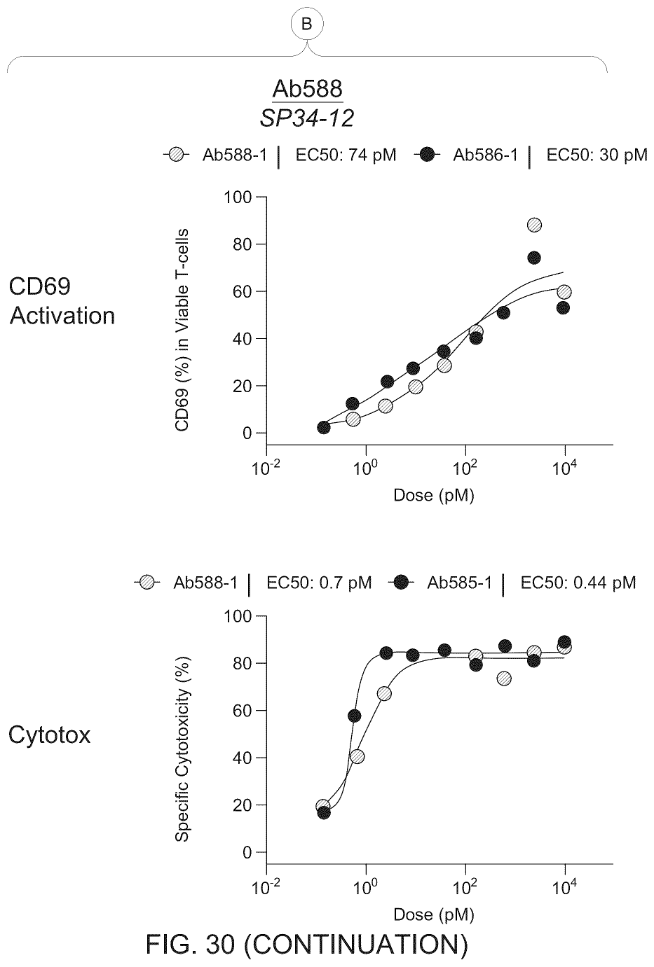

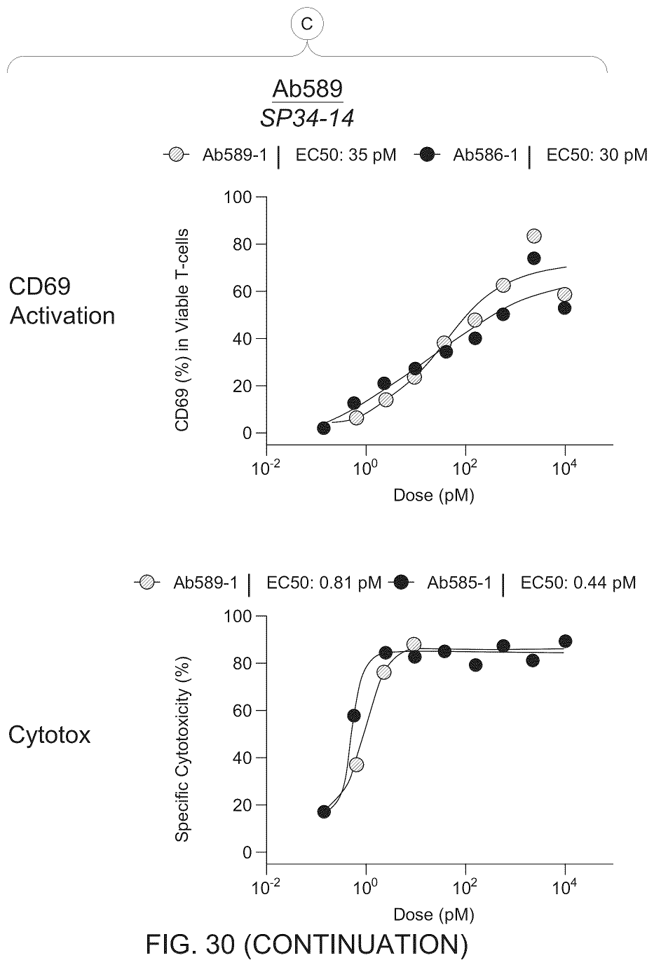

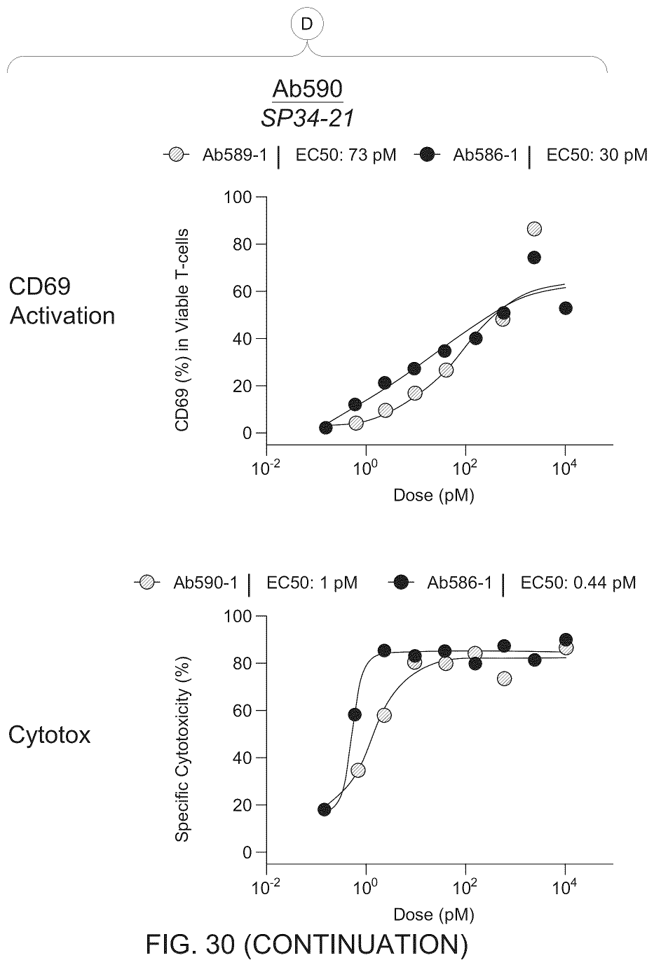

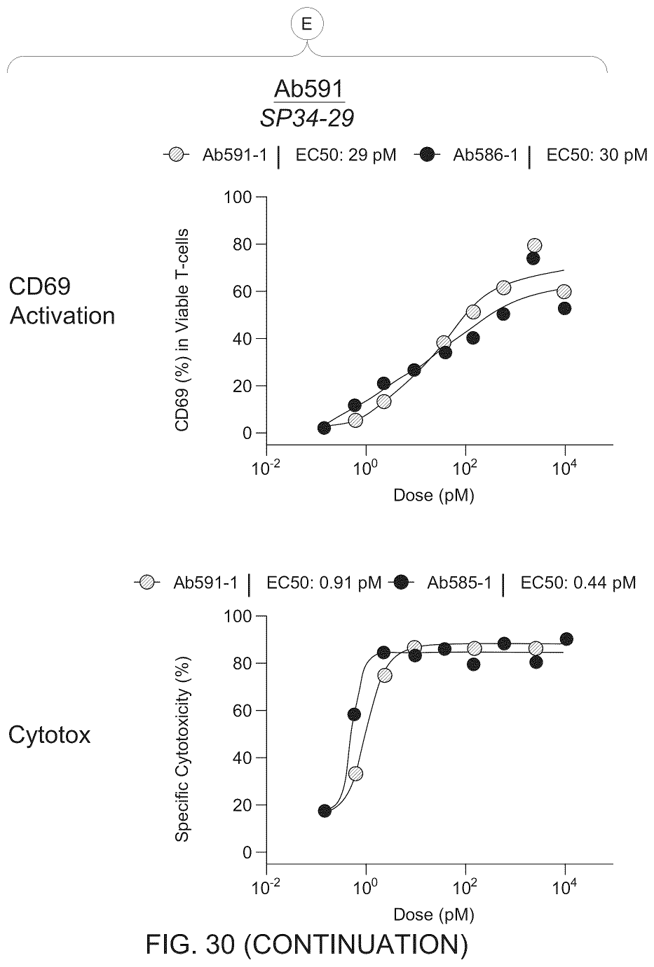

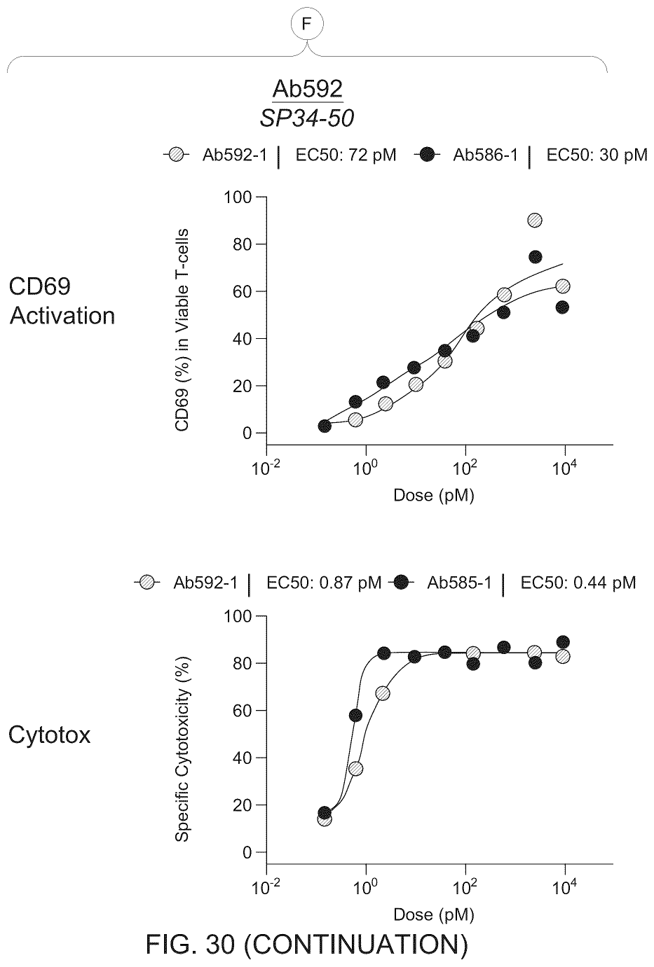

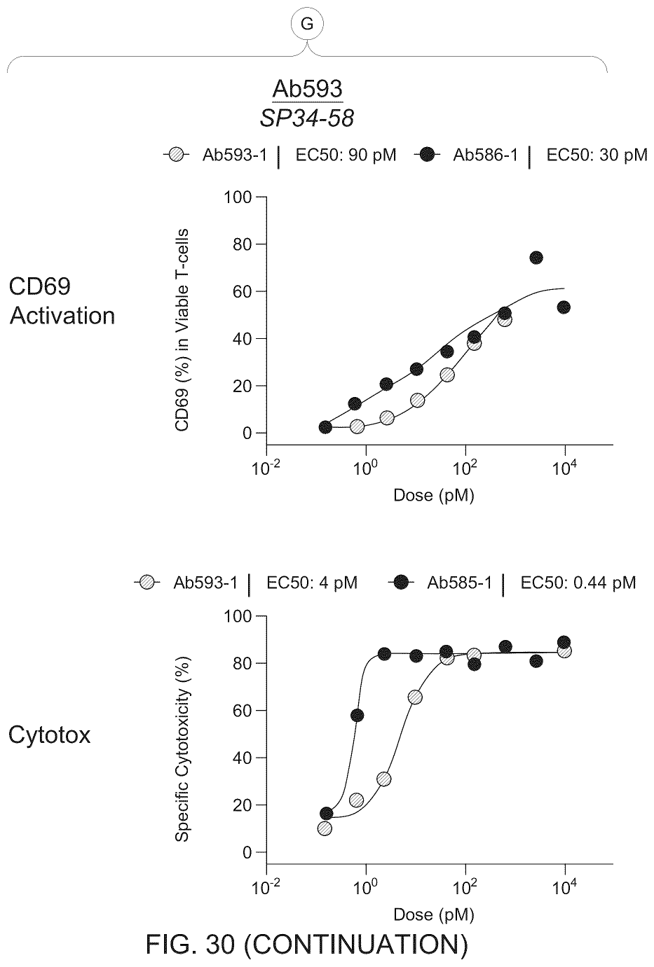

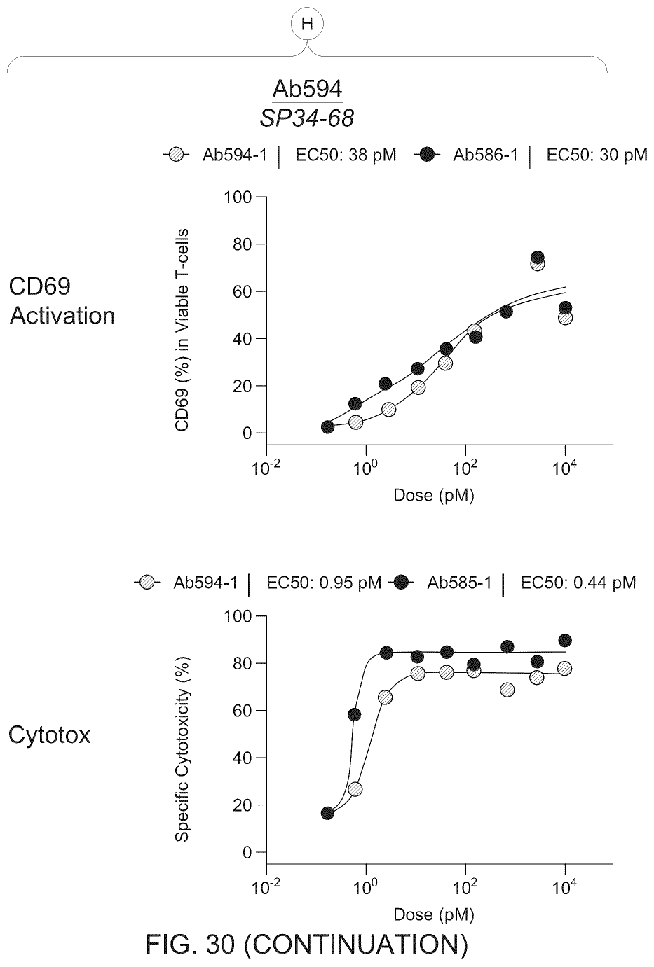

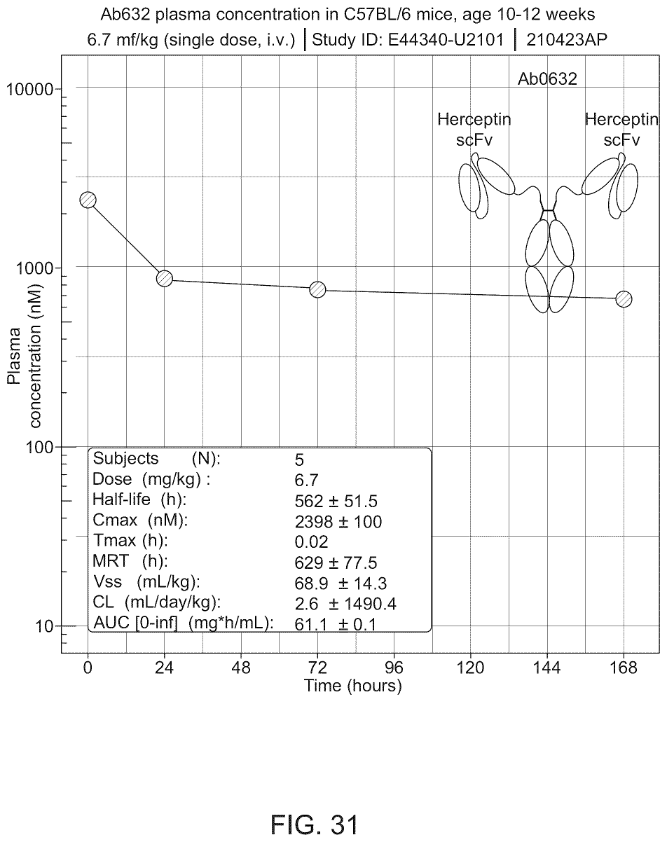

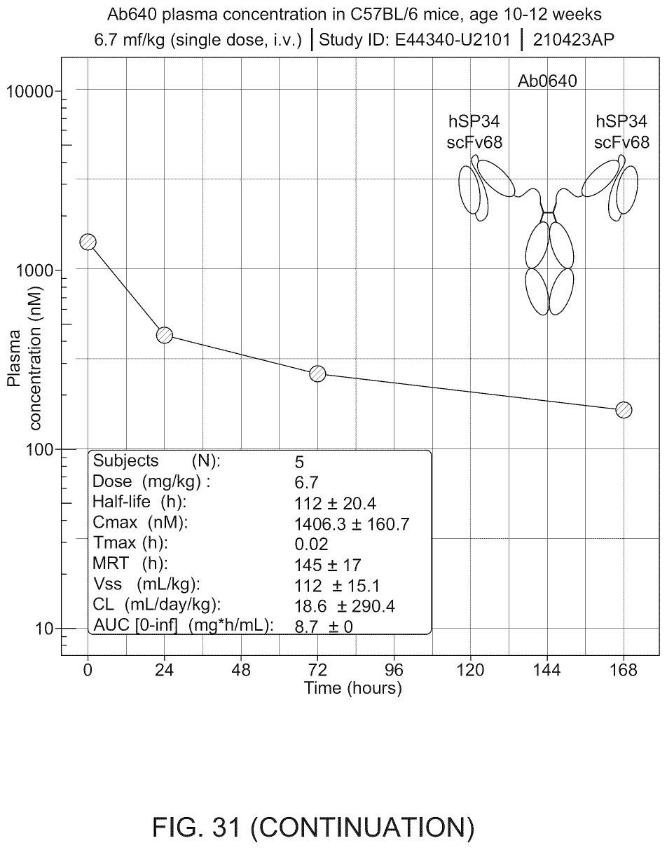

In some embodiments, Fc domains used herein include one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In some embodiments, particularly in the use of immunomodulatory proteins, it is desirable to ablate FcγRIIIa binding to eliminate or significantly reduce ADCC activity such that one of the Fc domains comprises one or more Fcγ receptor ablation variants. These ablation variants are depicted in FIG. 31 of U.S. Pat. No. 10,259,887, which is herein incorporated by reference in its entirety, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, according to the EU index. It should be noted that the ablation variants referenced herein ablate FcγR binding but generally not FcRn binding.

### 3. Domain Linkers

In many embodiments herein, domain linkers are used to link the various domains together in the CC and CT binding proteins. As will be appreciated by those in the art, the length and amino acid composition of the domain linker can vary depending on which domains are to be linked using the domain linker.

a. scFv Linkers

In some embodiments, the domain linker serves to link the VH and VL domains of an Fv together to form a scFv, and can be referred to as a “scFv linker”. In these embodiments, the scFv linker is long enough to allow the VH and VL domains to properly associate such that the VH and VL will self-assemble to form a scFv.

As is known in the art, the amino acid composition of the scFv linkers is selected to confer flexibility yet do not interfere with the variable domains to allow inter-chain folding to bring the two variable domains together to form a functional antigen binding site. In some embodiments, a scFv linker comprises glycine and serine residues. The amino acid sequence of the scFv linkers can be optimized, for example, by phage-display methods to improve the specific antigen binding and production yield of the scFv. In some embodiments, a scFv linker comprises glycine and serine residues. The amino acid sequence of the scFv linkers can be optimized, for example, by phage-display methods to improve the CD3 binding and production yield of the scFv.

Examples of peptide scFv linkers suitable for linking a variable light chain domain and a variable heavy chain domain in an scFv include but are not limited to (GS)n (SEQ ID NO: 3274), (GGS)n (SEQ ID NO: 3275), (GGGS)n (SEQ ID NO: 3276), (GGSG)n (SEQ ID NO: 3277), (GGSGG)n (SEQ ID NO: 3278), or (GGGGS)n (SEQ ID NO: 3279), wherein n is 1, 2, 3, 4, 5, 6, 7, 8, 9, or 10. In one embodiment, the scFv linker can be (GGGGS)₄ (SEQ ID NO: 3280) or (GGGGS)₃ (SEQ ID NO: 3281). Variation in the linker length may retain or enhance activity, giving rise to superior efficacy in activity studies. Accordingly, in some embodiments, the scFv linker is from 10 to 25 amino acids in length. In some embodiments, the peptide scFv linker is selected from GGGGSGGGGSGGGGS (SEQ ID NO: 3282), GGGGSGGGSGGGGSGGGGS (SEQ ID NO: 3283), GGSGGSGGSGGSGG (SEQ ID NO: 3284).

As discussed herein, the scFv domains can have either orientation, that is, from N- to C-terminal, VH-scFv linker-VL or VL-scFv linker-VH.

b. scFab Linkers

In some embodiments, domain linkers are used to link the light chain VL and CL with the VH and CH1 of the heavy chain to form a single chain Fab (scFab), referred to as a “scFab linker”. Generally, scFab linkers are selected that do not hinder antibody assembly or affect Fab binding affinity to antigens. In addition, the scFab linkers present minimal adverse effects of the linker sequence on the yield or folding of the Fab.

In some embodiments, the scFab linkers are polypeptide linkers with an amino acid sequence with a length of at least 30 amino acids, for example, between 32 to 80 amino acids, or between 34 to 60 amino acids. In one embodiment, the scFab linker is (GxS)nGm with G=glycine, S=serine, (x=3, n=8, 9 or 10 and m=0, 1, 2 or 3) or (x=4 and n=6, 7 or 8 and m=0, 1, 2 or 3), preferably with x=4, n=6 or 7 and m=0, 1, 2 or 3, more preferably with x=4, n=7 and m=2. In one embodiment, the scFab linker is (GGGGS)₆G2.

In some embodiments, the natural intermolecular disulfide bond between CL and CH1 in scFab is deleted.

In some embodiments, a disulfide bond is introduced into VH and VL to further disulfide stabilization of the scFab. In one embodiment, the optional disulfide bond introduced is between VH at position 44 and VL at position 100. In one embodiment, the optional disulfide bond introduced is between VH at position 105 and VL at position 43 (numbering always according to EU index of Kabat).

Configurations of an scFab can include VH-CH1-linker-VL-CL, VL-CL-linker-VH-CH1, VH-CL-linker-VL CH1 and VL-CH1-linker-VH-CL.

c. General Domain Linkers

Aside from scFv linkers and scFab linkers, other domain linkers are used in linking two or more domains in this invention, for example, to connect the CID domains and the CD3 or TTA antigen binding domains. A domain linker may have a length that is adequate to link two domains in such a way that they assume the correct conformation relative to one another so that they retain the desired activity. In general, a linker joining two domains can be designed to (1) allow the two domains to fold and act independently of each other, (2) not have a propensity for developing an ordered secondary structure which could interfere with the functional domains of the two domains, (3) have minimal hydrophobic or charged characteristic which could interact with the functional protein domains and/or (4) provide steric separation of the two domains.

The length and composition of a domain linker can be varied considerably provided that it can fulfill its purpose as a molecular bridge. The length and composition of the linker are generally selected taking into consideration the intended function of the linker, and optionally other factors such as ease of synthesis, stability, resistance to certain chemical and/or temperature parameters, and biocompatibility.

For example, a domain linker may be a peptide which includes the following amino acid residues: Gly, Ser, Ala, or Thr. In some embodiments, the linker peptide is from about 1 to 50 amino acids in length, about 1 to 30 amino acids in length, about 1 to 20 amino acids in length, or about 5 to about 10 amino acids in length. In some embodiments, the peptide domain linker is (GXS)n or (GXS)nGm with G-glycine, S-serine, and (x=3, n=3, 4, 5 or 6, and m=0, 1, 2 or 3) or (x=4, n=2,3,4 or 5 and m=0, 1, 2 or 3). Exemplary peptide linkers include glycine-serine polymers such as (GS)n, (GGS)n, (GGGS)n, (GGSG)n (GGSGG)n, (GSGGS)n, and (GGGGS)n, wherein n is an integer of at least one (e.g., 1, 2, 3, 4, 5, 6, 7, 8, 9, or 10); glycine-alanine polymers; alanine-serine polymers; and other flexible linkers.

Alternatively, a variety of non-proteinaceous polymers can be used as a domain linker, including but not limited to polyethylene glycol (PEG), polypropylene glycol, polyoxyalkylenes, or copolymers of polyethylene glycol and polypropylene glycol.

A domain linker may also be derived from immunoglobulin light chain, for example Cκ or Cλ. Linkers can also be derived from immunoglobulin heavy chains of any isotype, including for example Cγ1, Cγ2, Cγ3, Cγ4, Cα1, Cα2, Cδ, Cε, and Cμ. For example, domain linkers can include any sequence of any length of CL/CH1 domain but not all residues of CL/CH1 domain; for example, the first 5-12 amino acid residues of the CL/CH1 domains.

A domain linker may also be derived from other proteins such as Ig-like proteins (e.g., TCR, FcR, KIR), hinge region-derived sequences, and other natural sequences from other proteins.

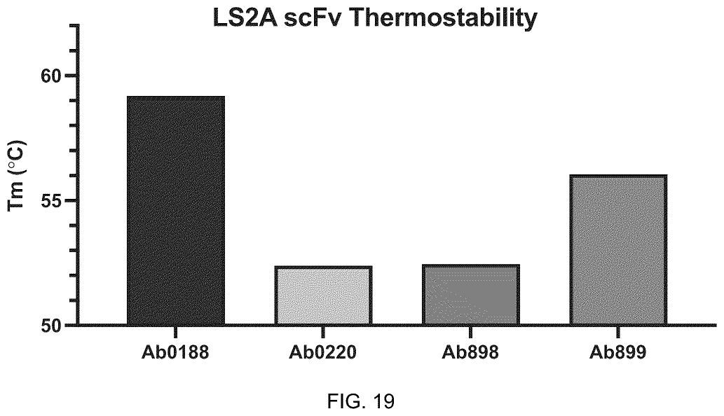

In some embodiments, the hinge domain of a human IgG antibody is used as a linker. The hinge domains of human IgG1, IgG2, IgG3 and IgG4 are shown in FIG. 19. In some cases, the hinge domain can contain amino acid substitutions as well. For example, a hinge domain from IgG4 comprising a S228P variant can be used. In some embodiments, the domain linker is a combination of a hinge domain and a flexible linker.

### 4. Anti-CD3 Antigen Binding Domains (αCD3 ABDs)

The T cell engaging activity of the CC binding proteins is achieved by incorporating an anti-CD3 antigen binding domain (αCD3 ABD) into the CC binding proteins.

As a part of the TCR, CD3 is a protein complex that includes a CD3λ (gamma) chain, a CD3δ (delta) chain, and two CD3ε (epsilon) chains which are present on the cell surface. CD3 associates with the α (alpha) and β (beta) chains of the TCR as well as CD3 (zeta) altogether to form the complete TCR. Clustering of CD3 on T cells, such as by immobilized anti-CD3 antibodies, leads to T cell activation similar to the engagement of the T cell receptor but independent of its clone typical specificity.

In some embodiments, the CC binding proteins described herein comprise an antigen binding domain which specifically binds to human CD3ε.

In some embodiments, the αCD3 ABD is derived from a monoclonal antibody, a polyclonal antibody, a recombinant antibody, a human antibody, or a humanized antibody. The αCD3 ABD can take any format, including but not limited to an Fv, an scFv, and an sdAb such as the VHH domain of a camelid derived sdAb and scFab.

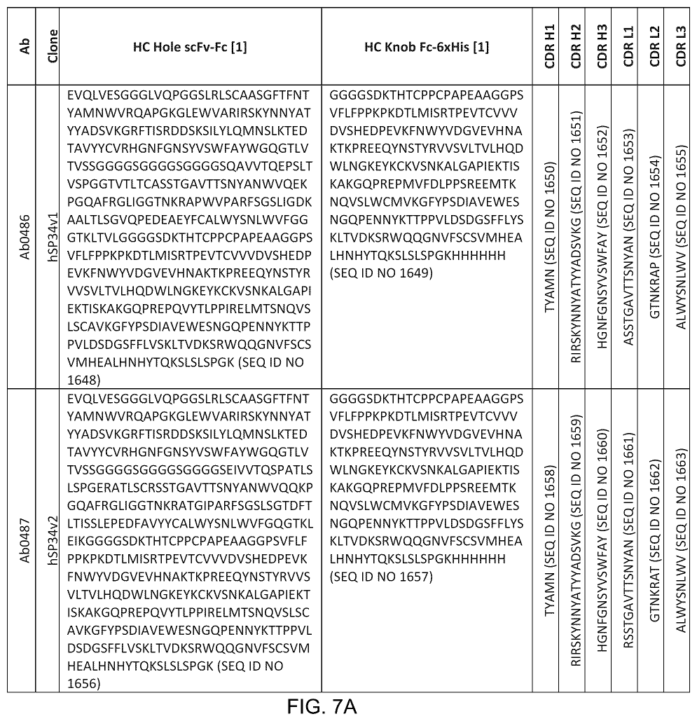

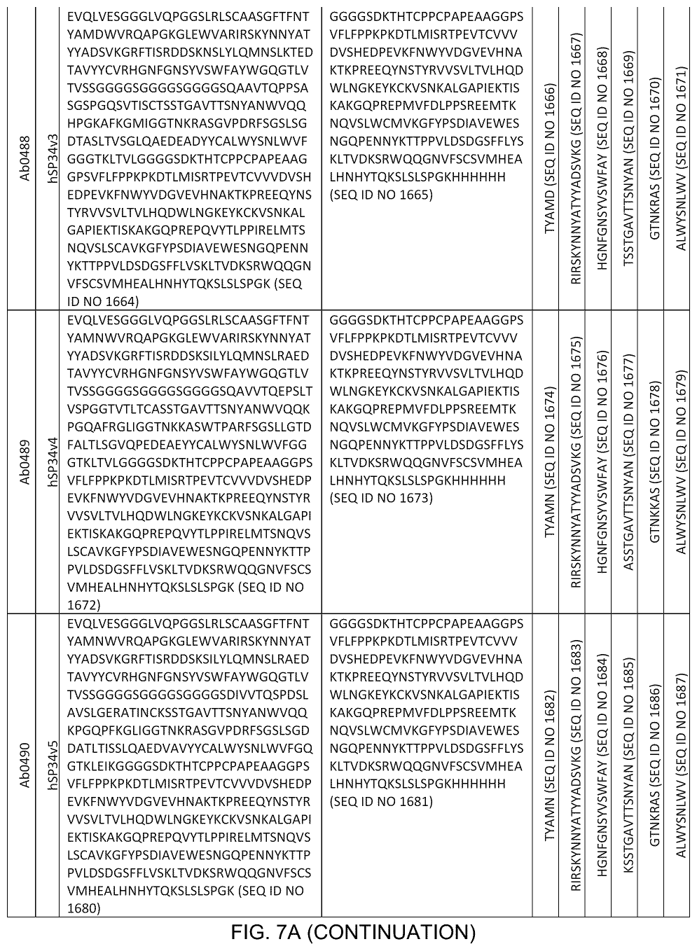

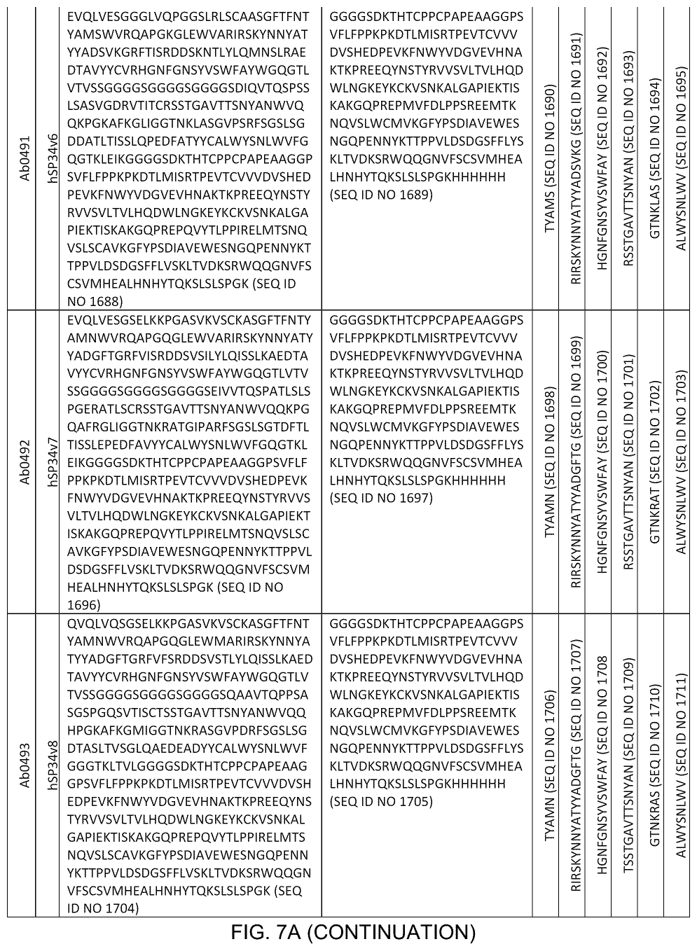

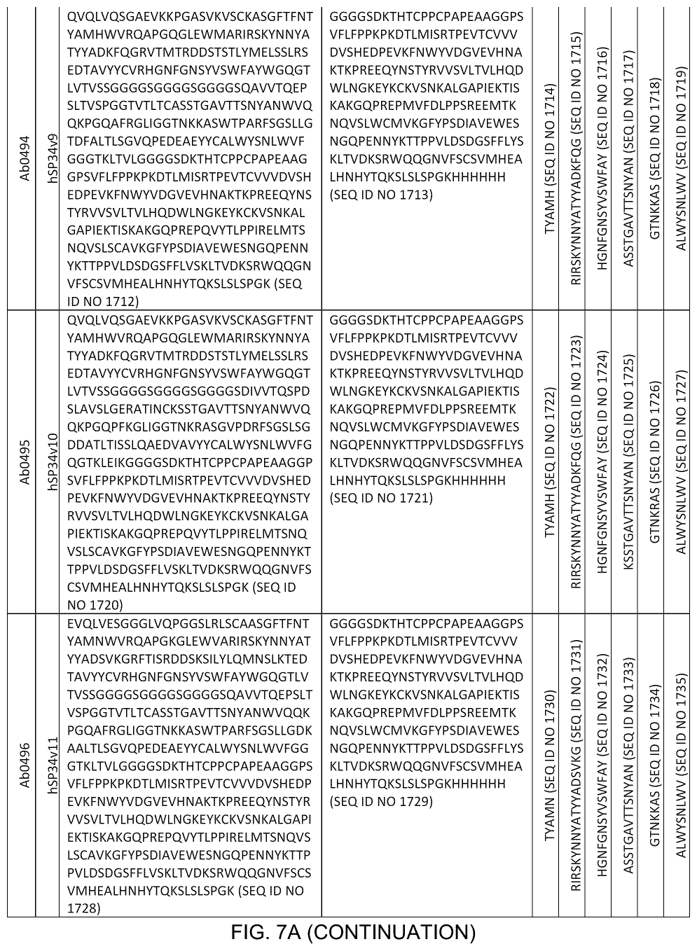

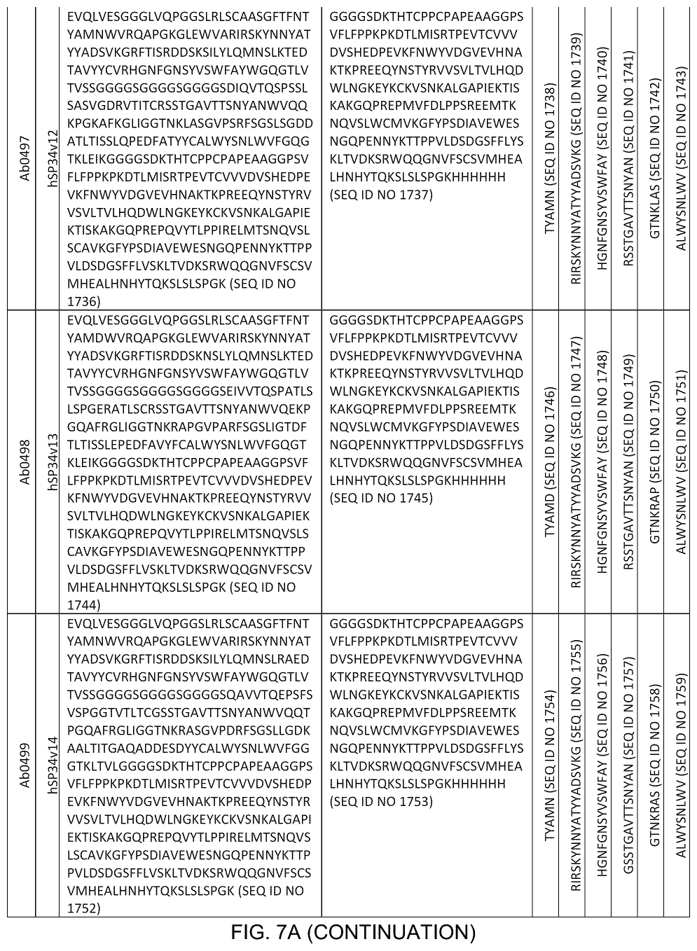

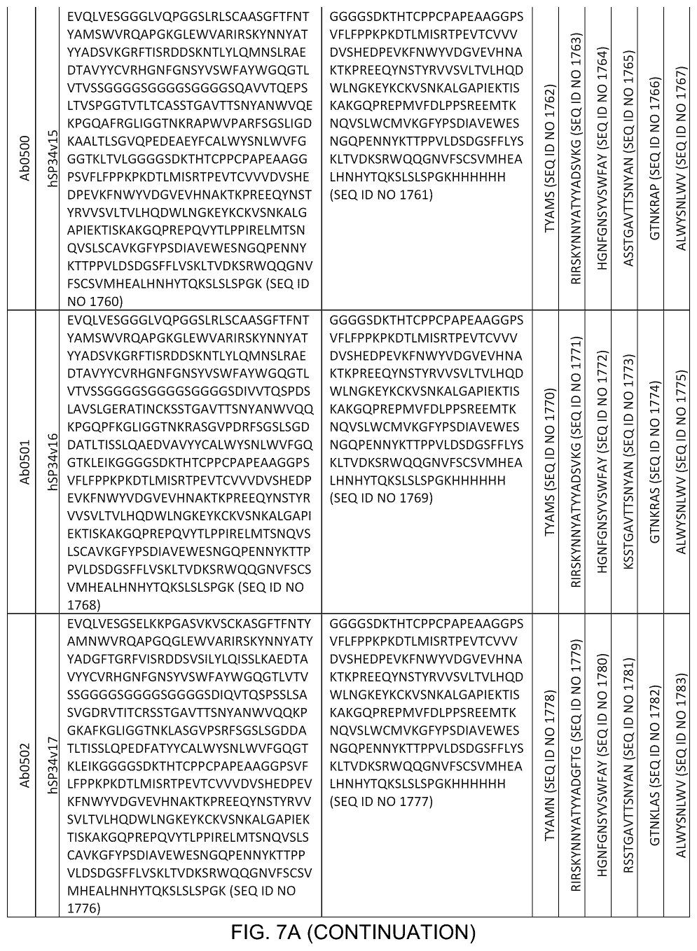

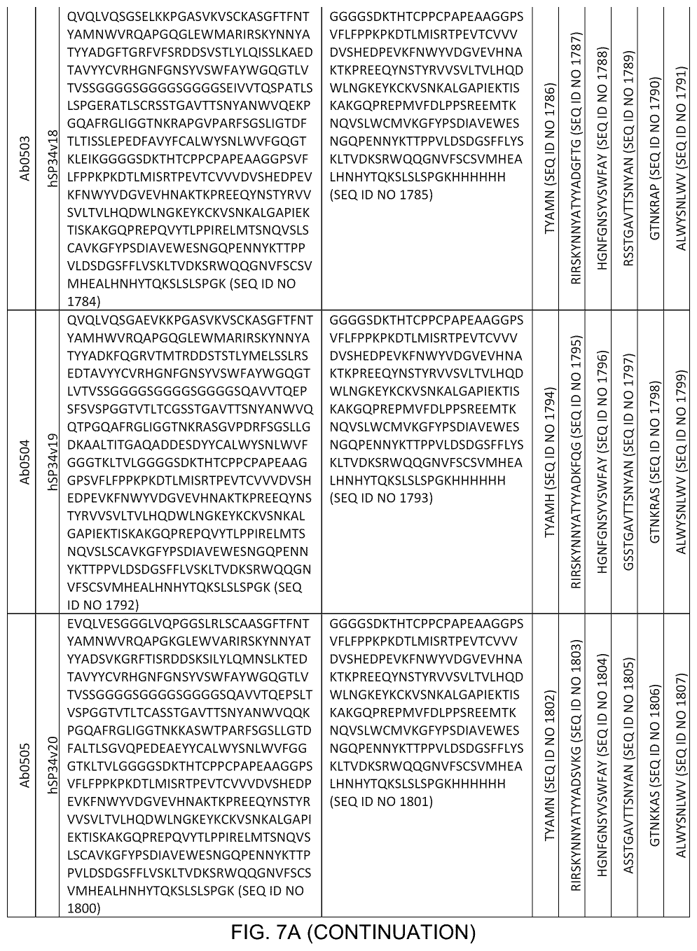

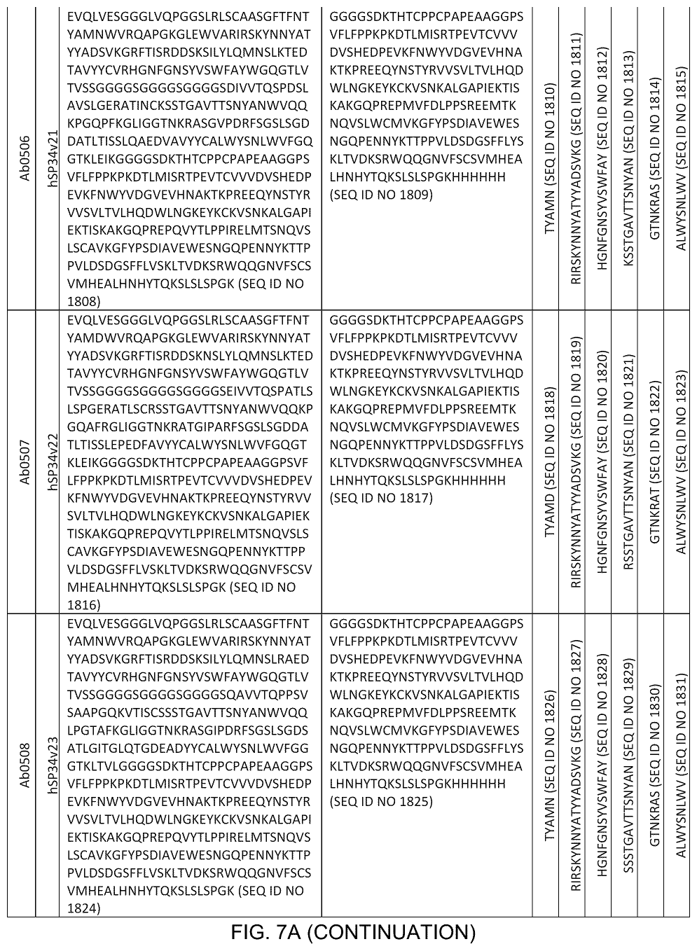

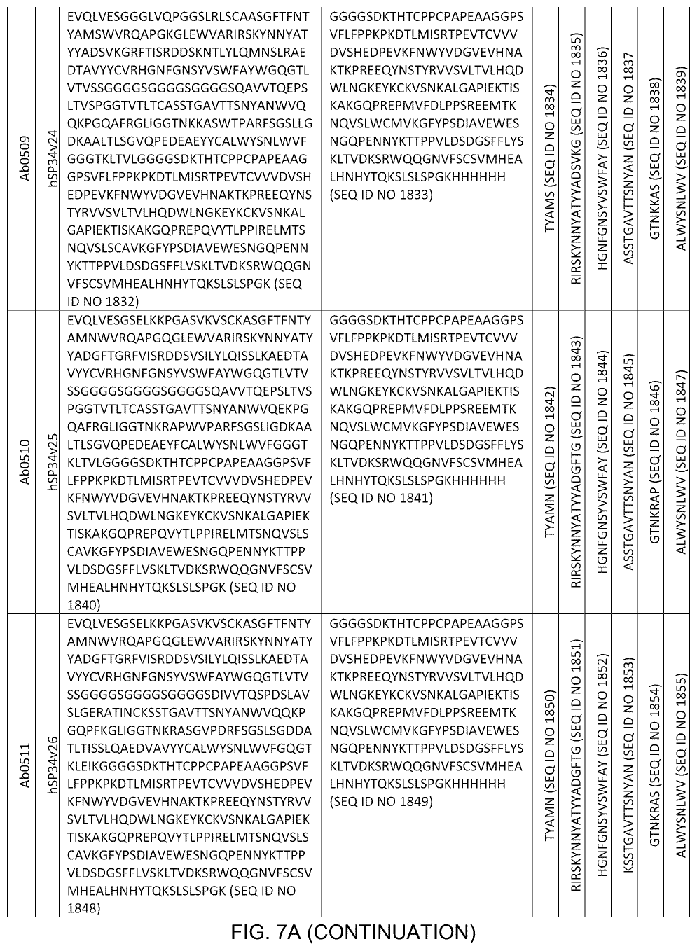

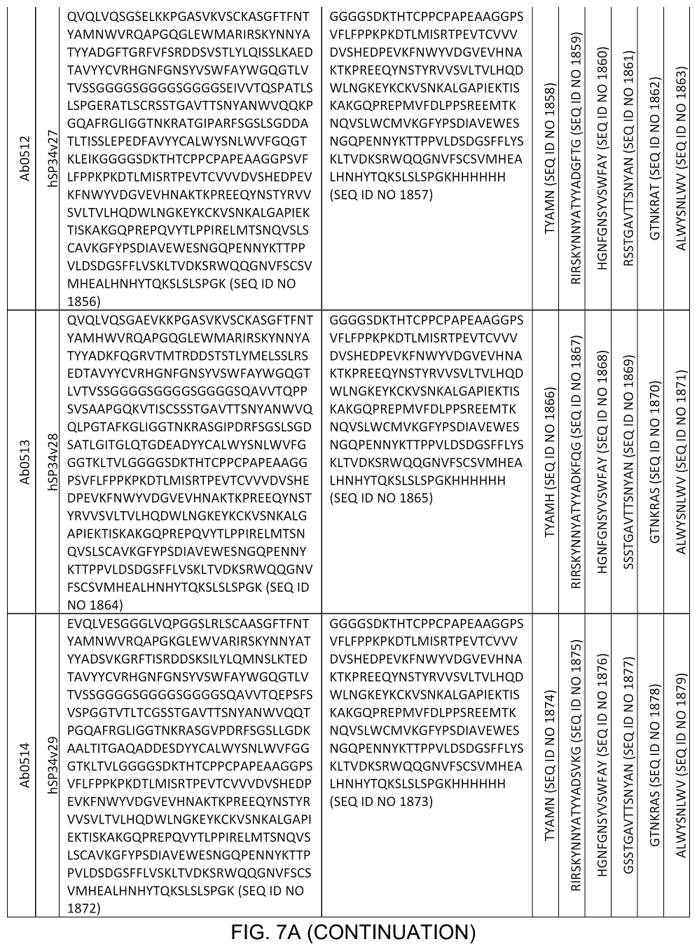

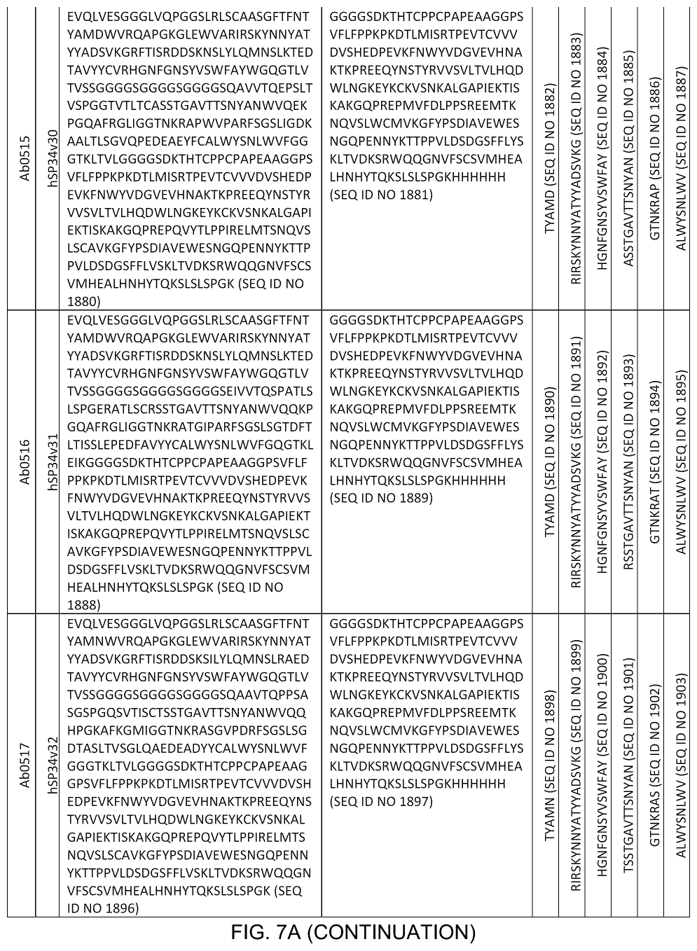

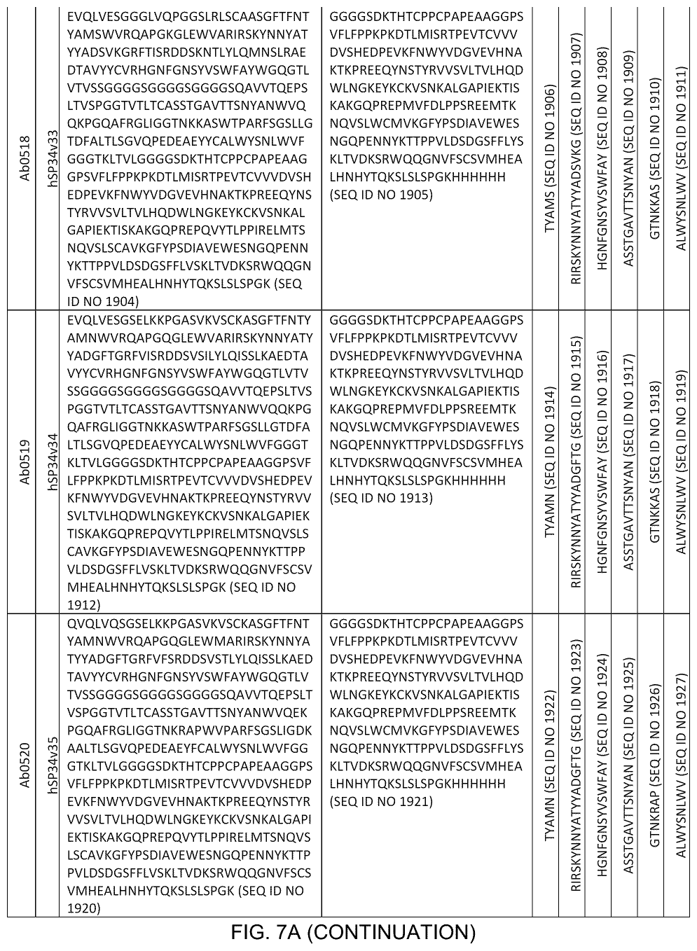

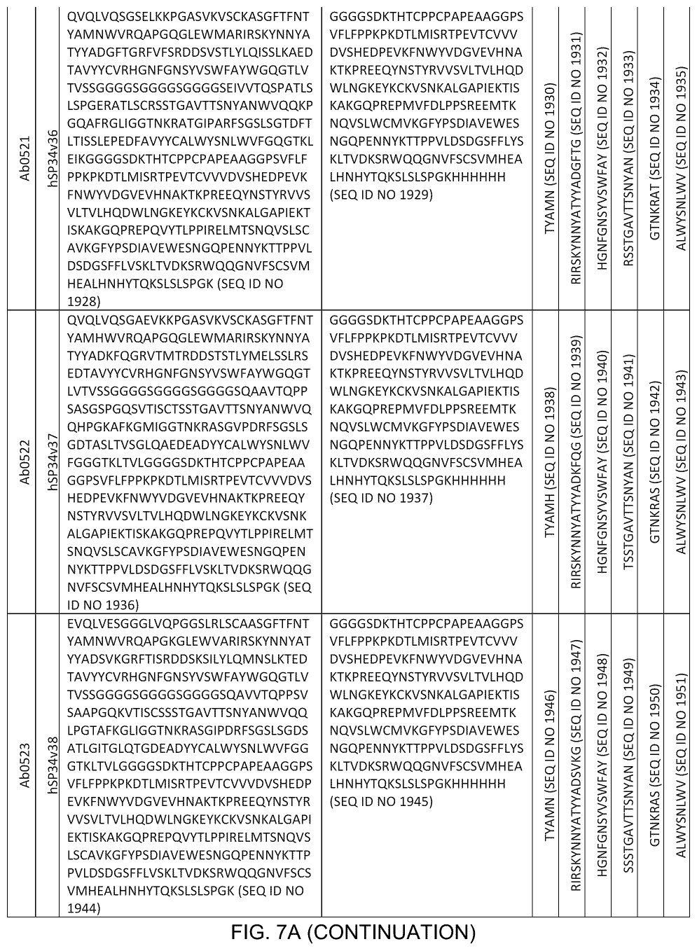

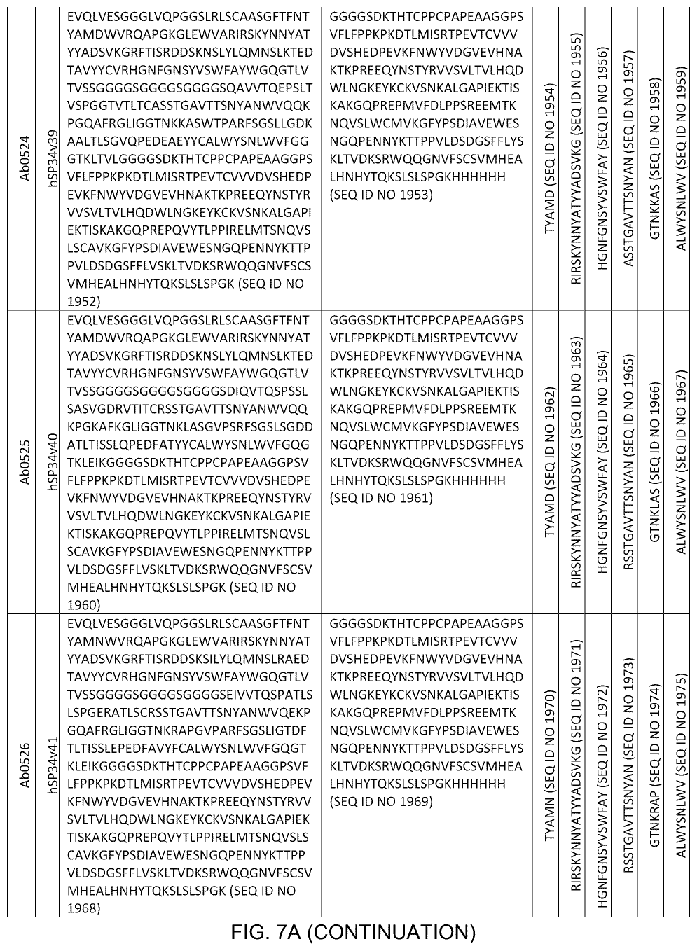

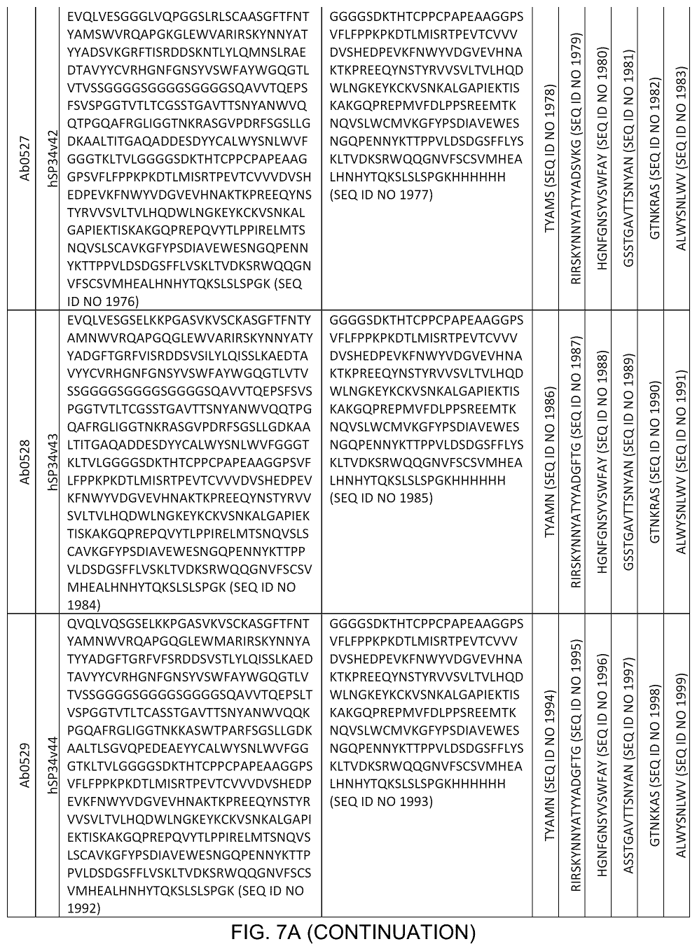

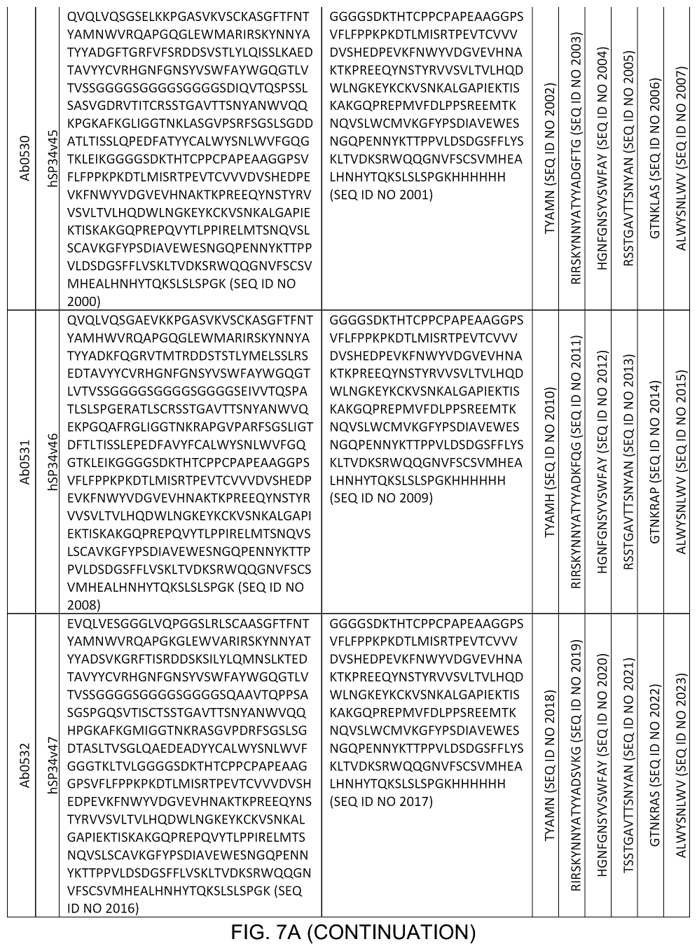

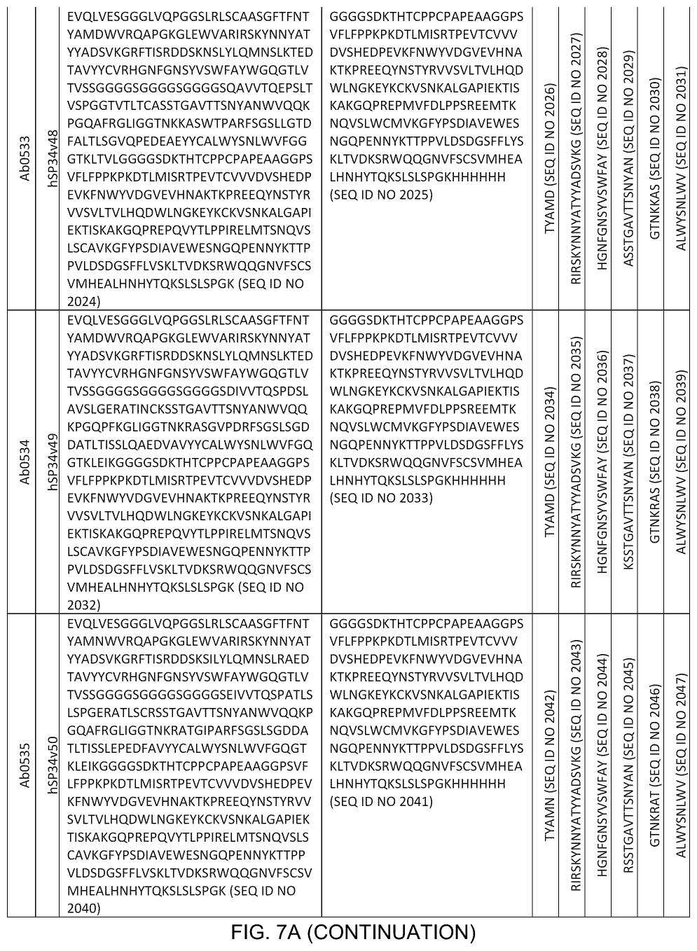

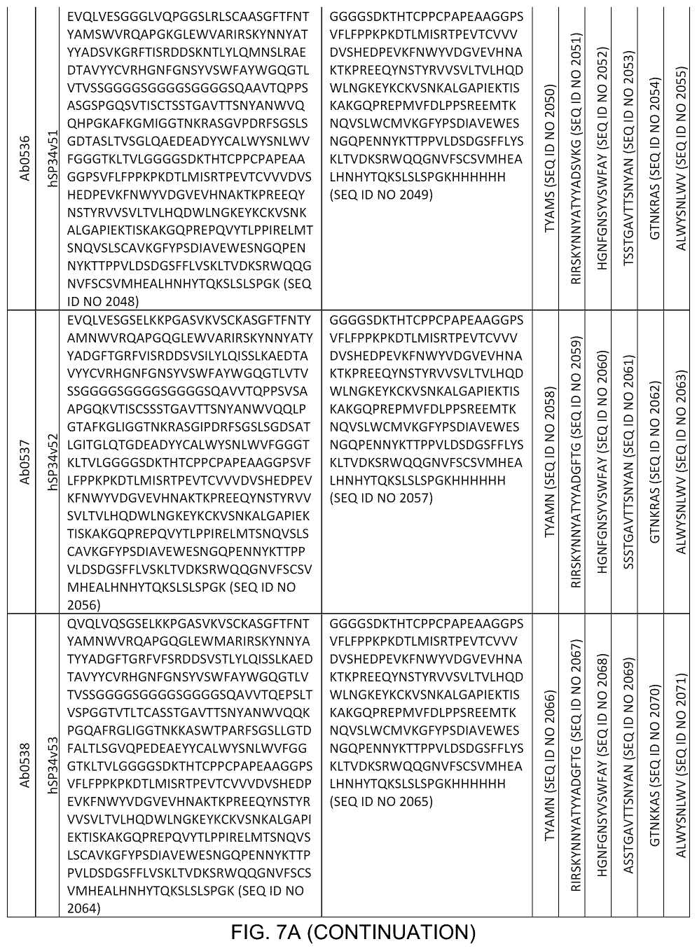

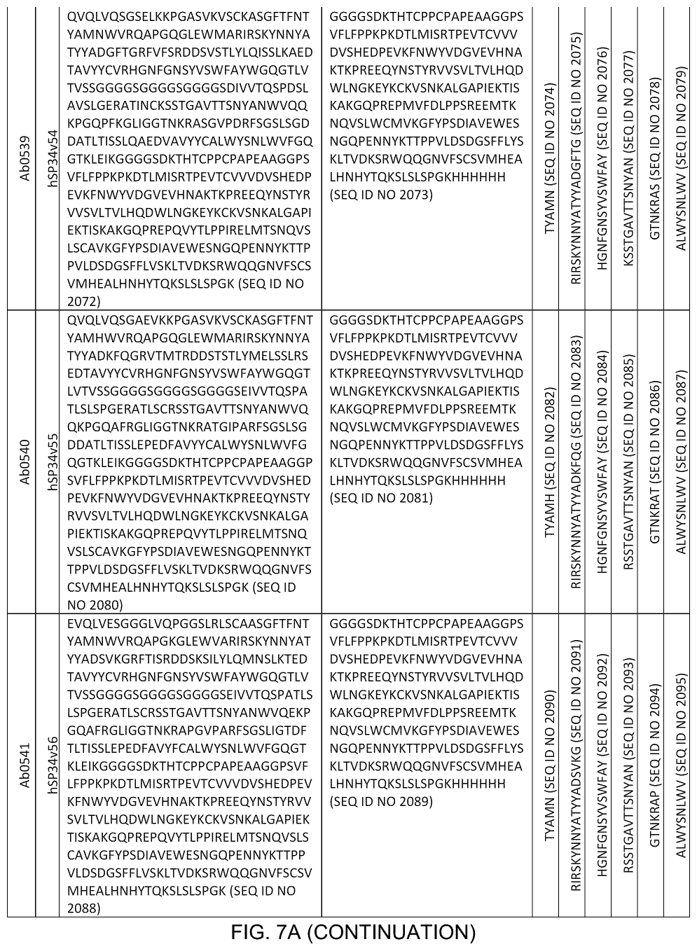

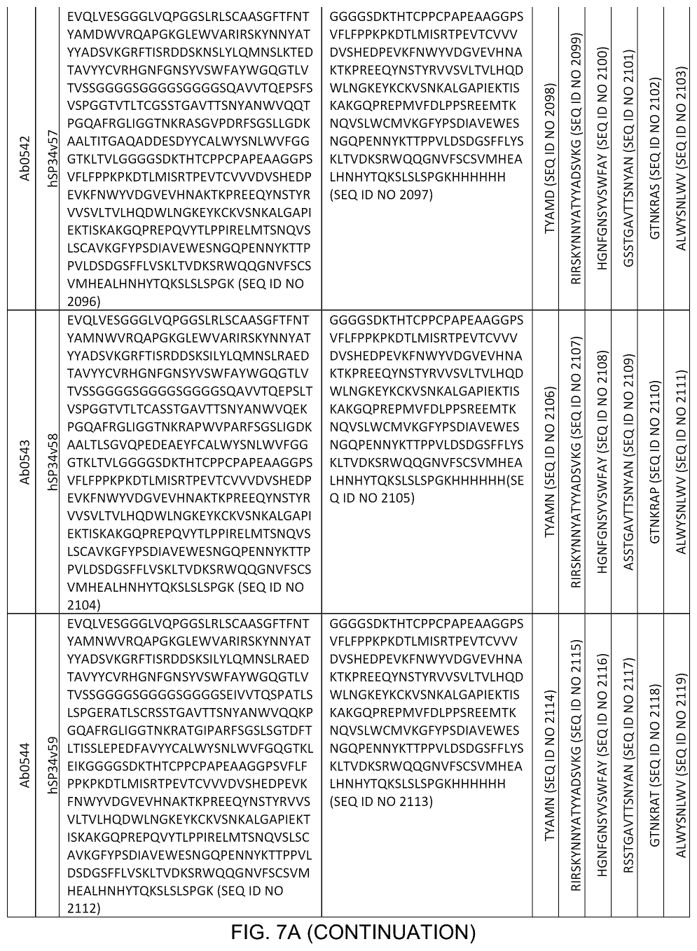

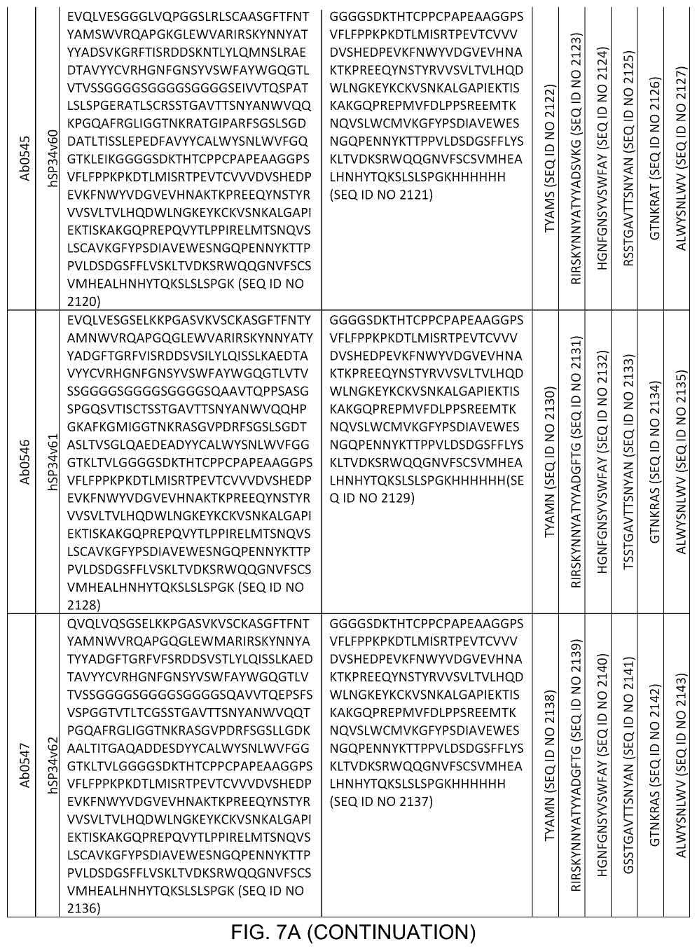

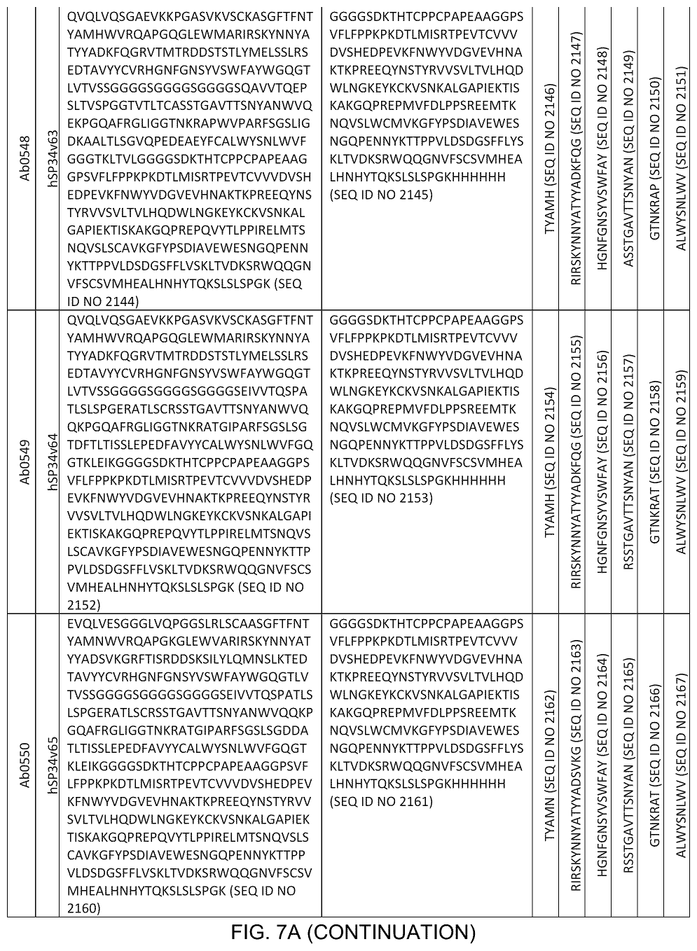

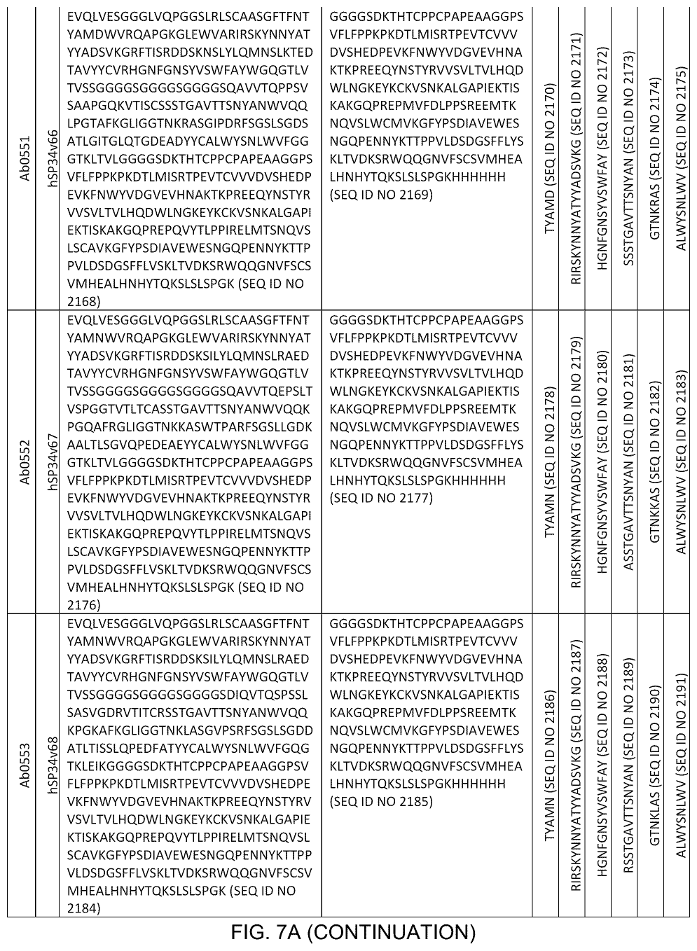

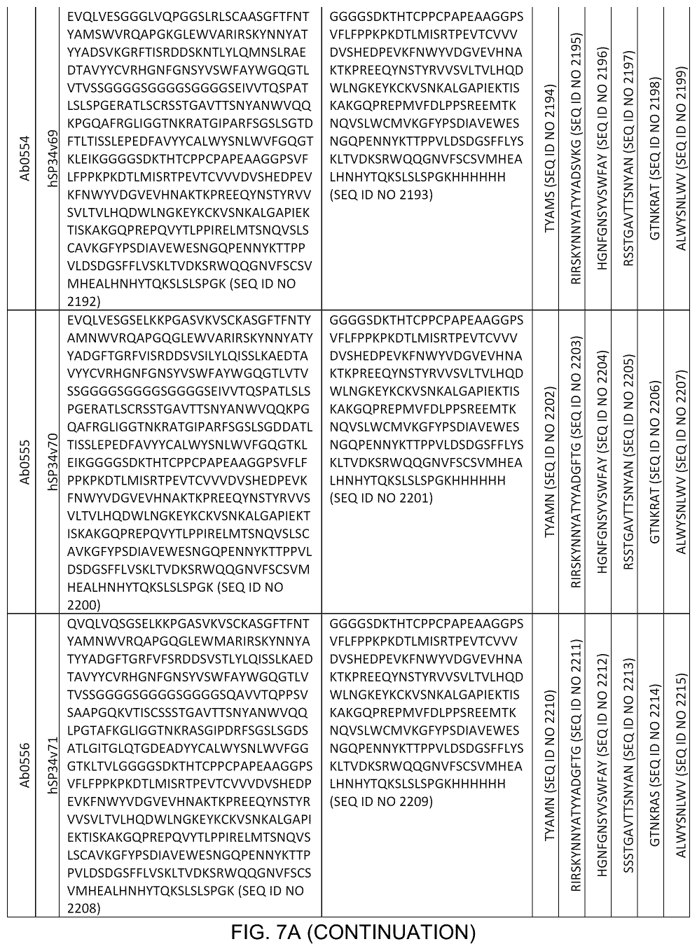

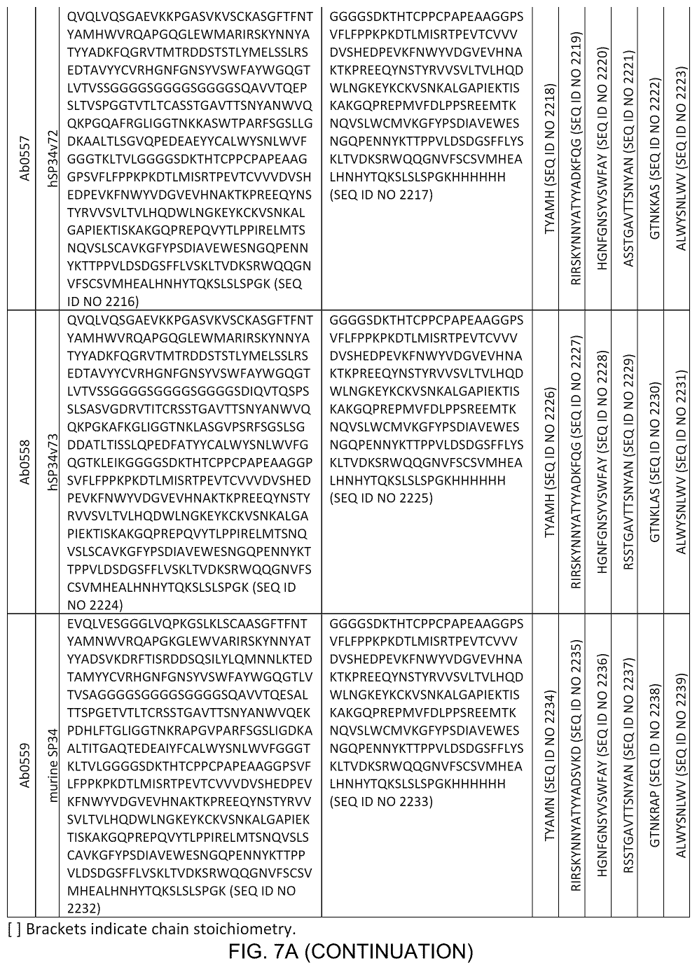

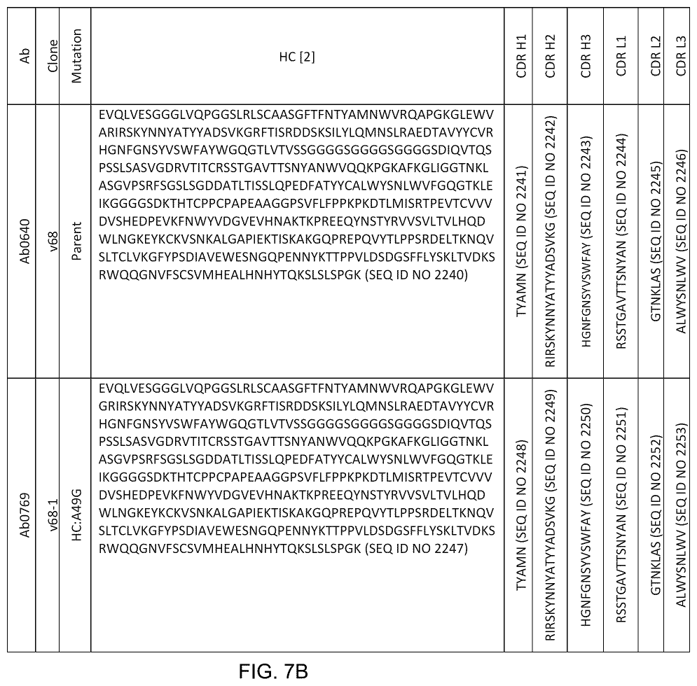

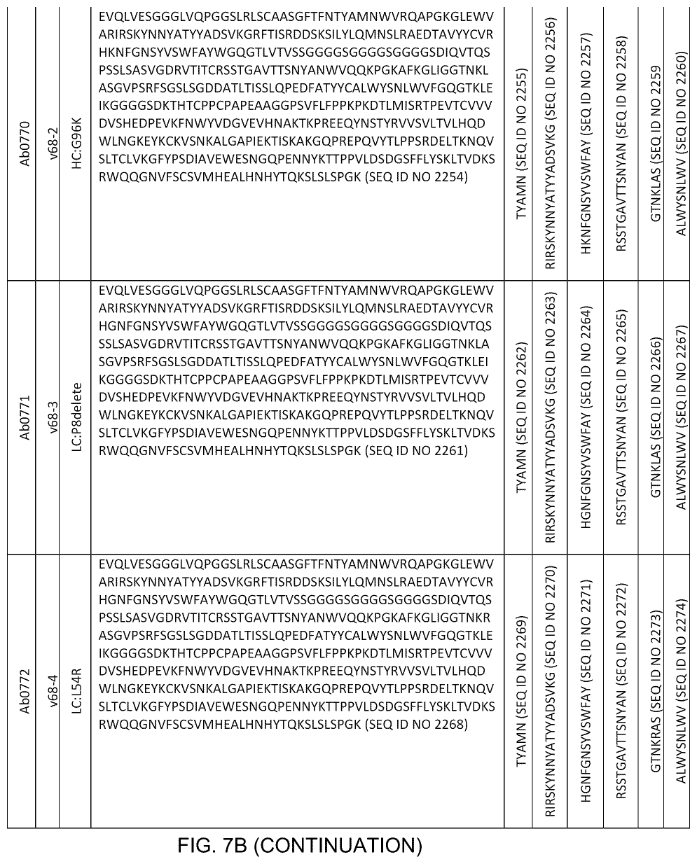

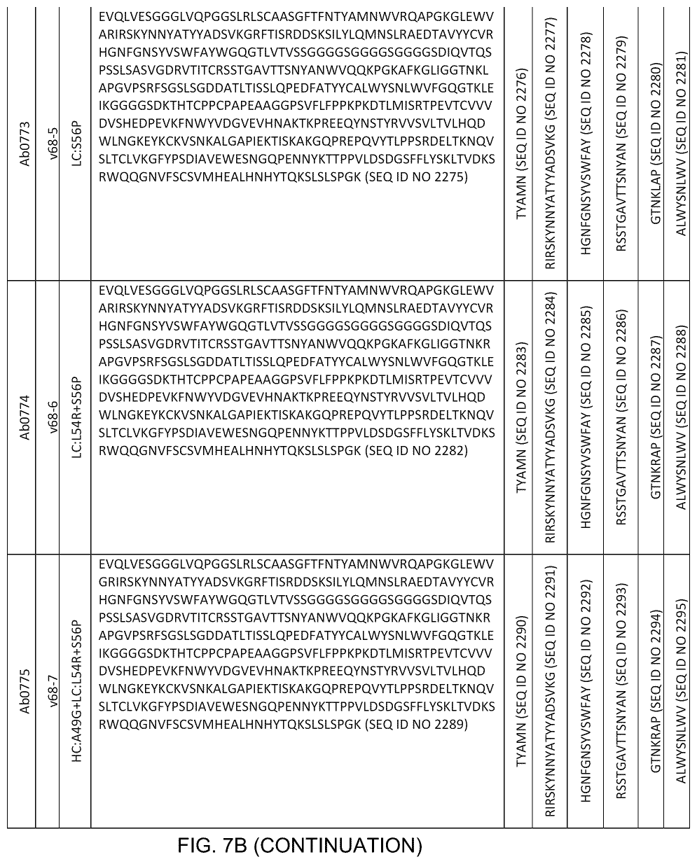

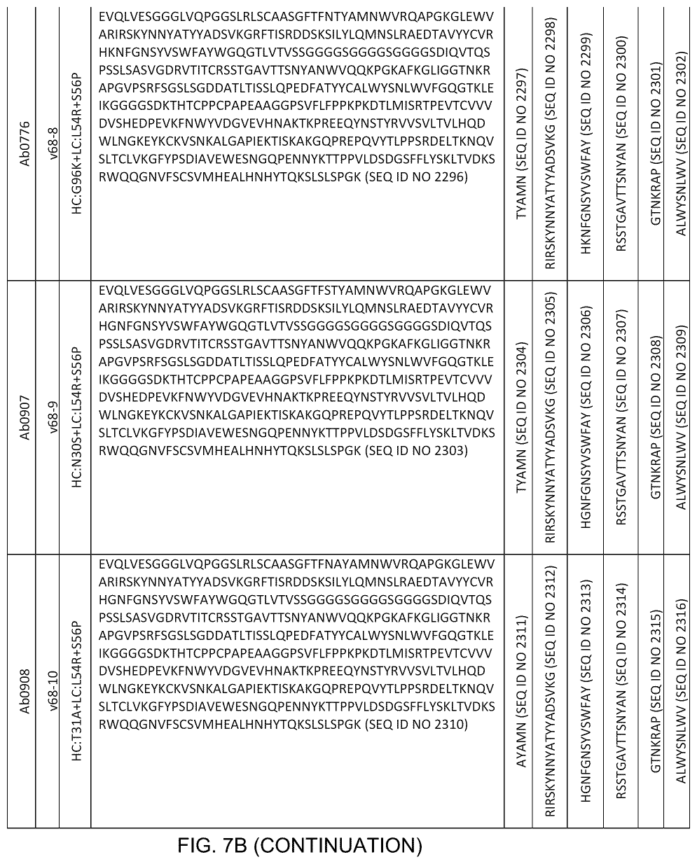

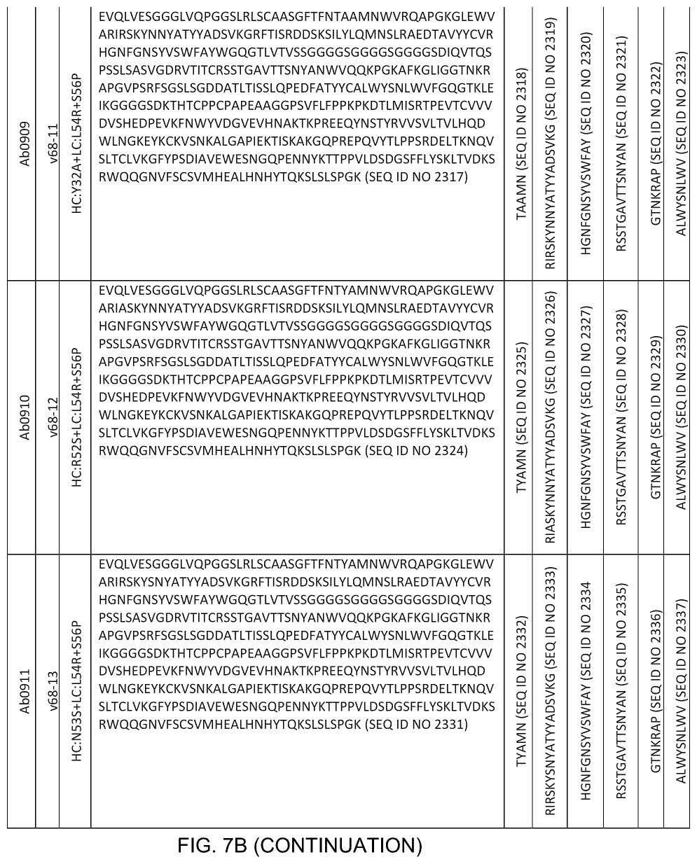

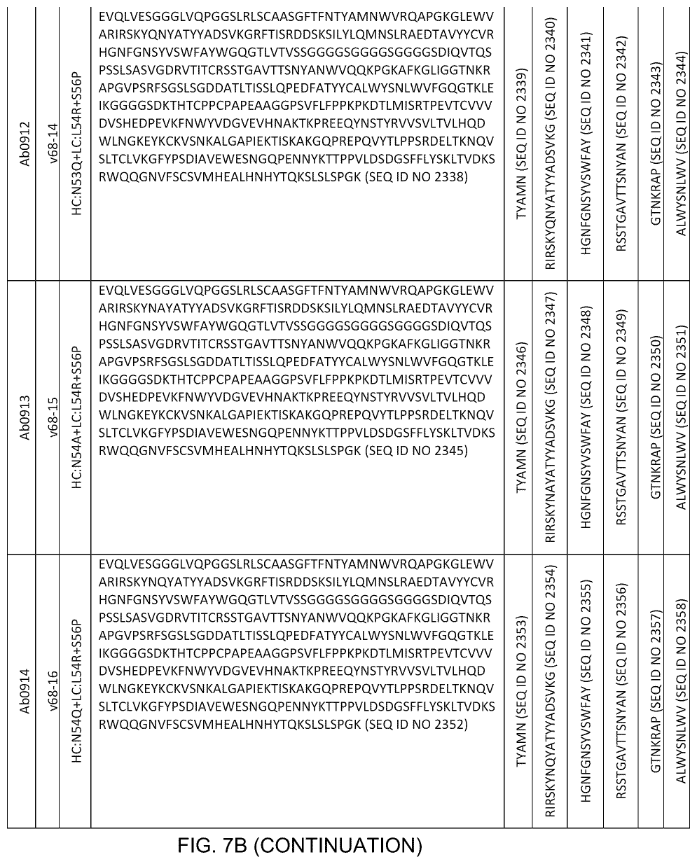

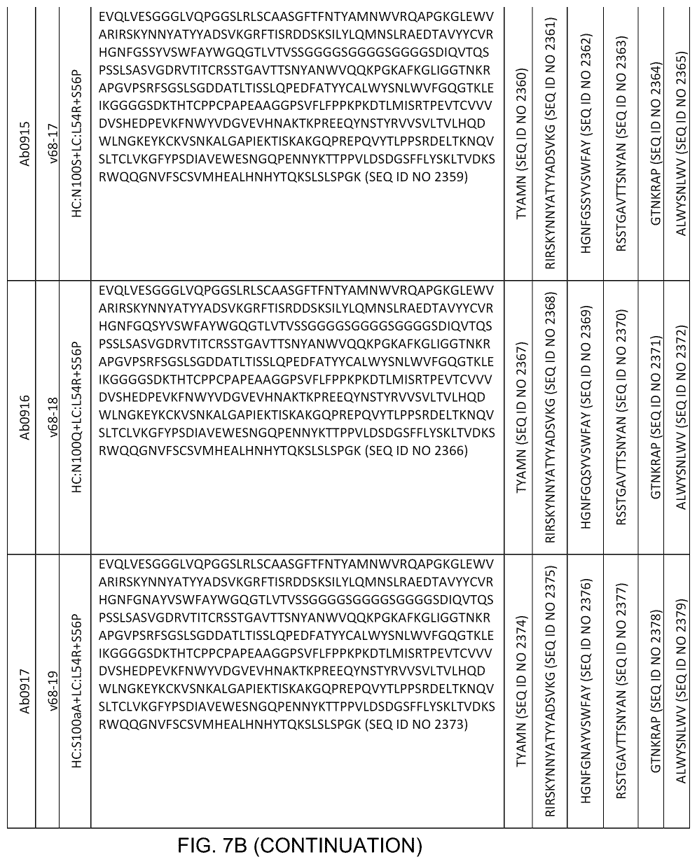

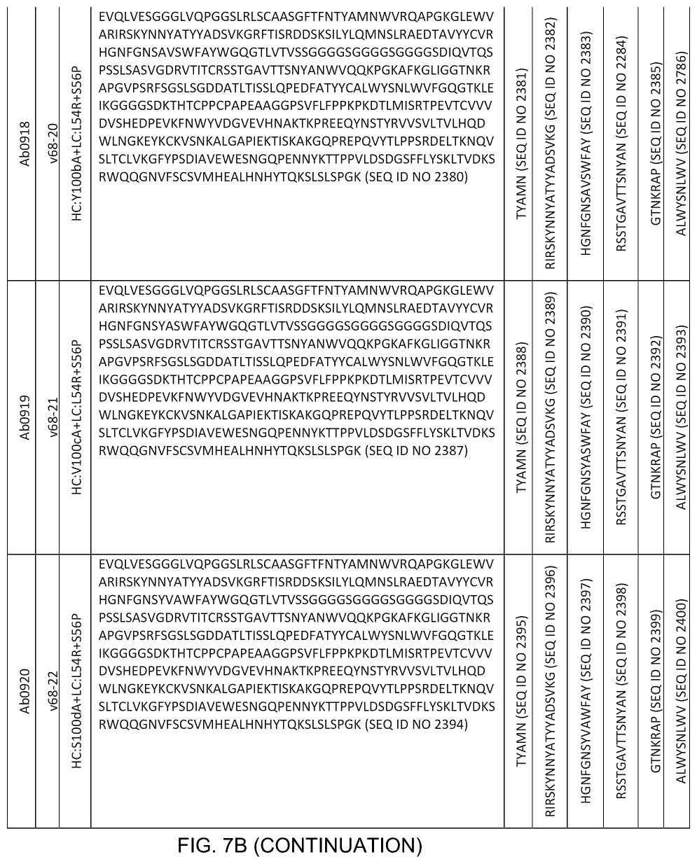

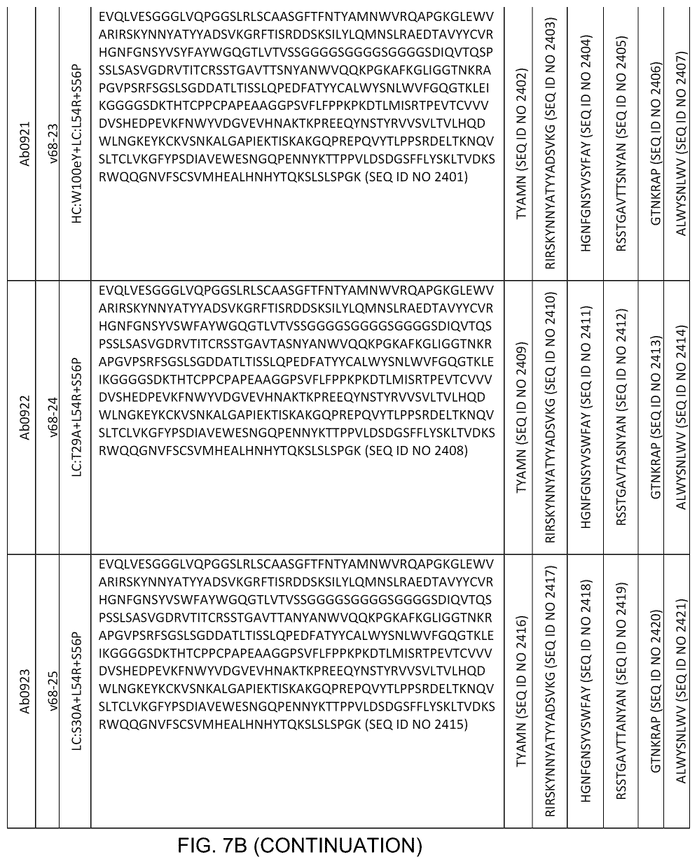

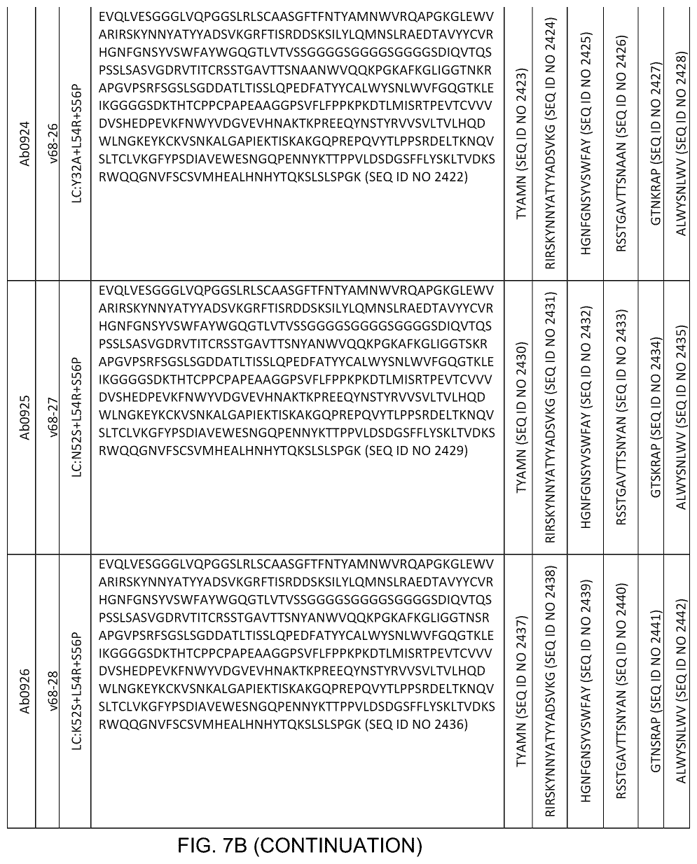

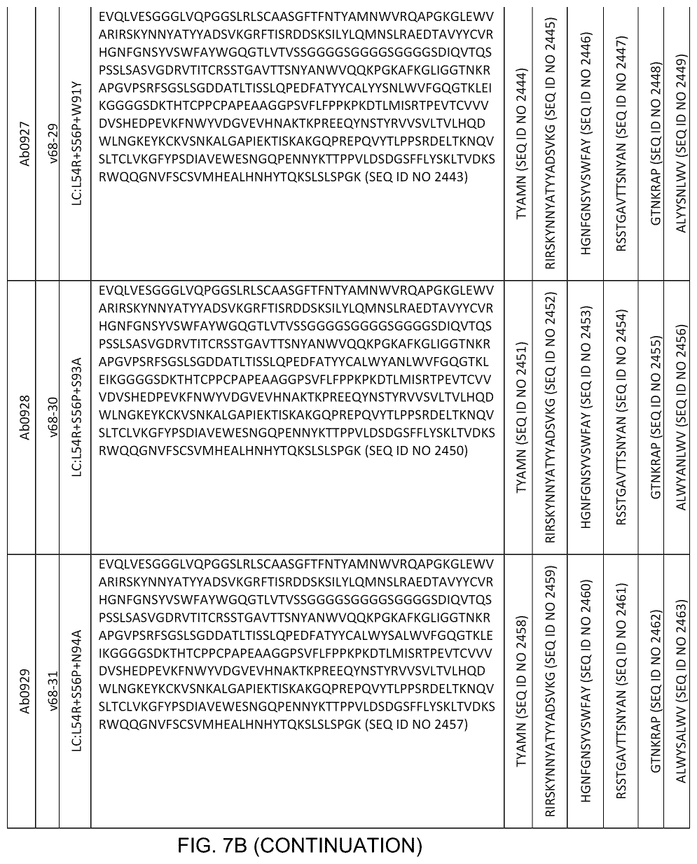

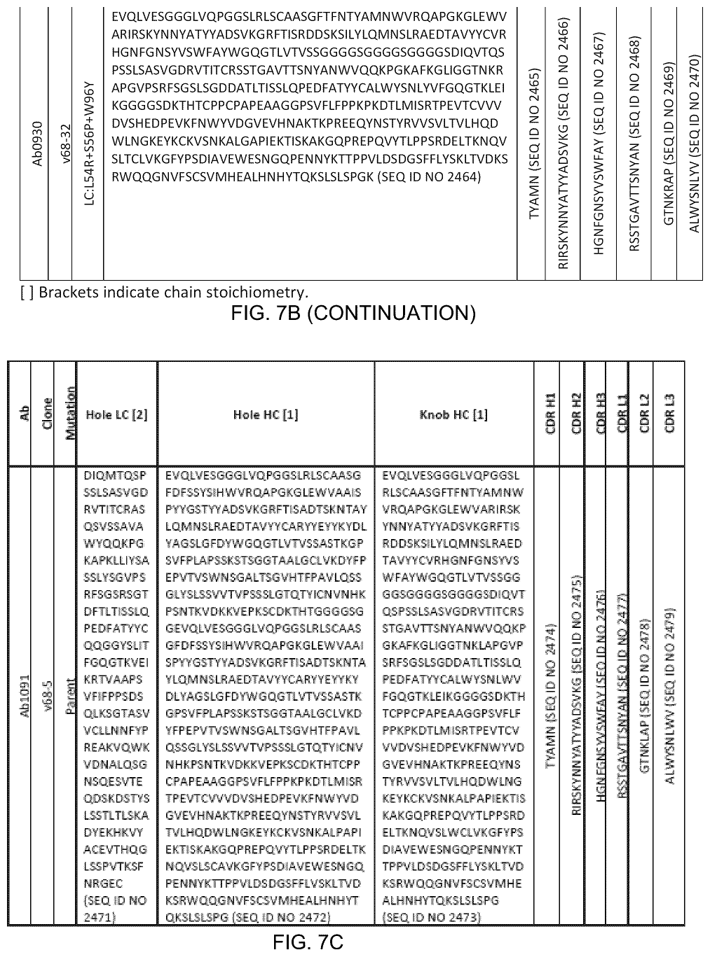

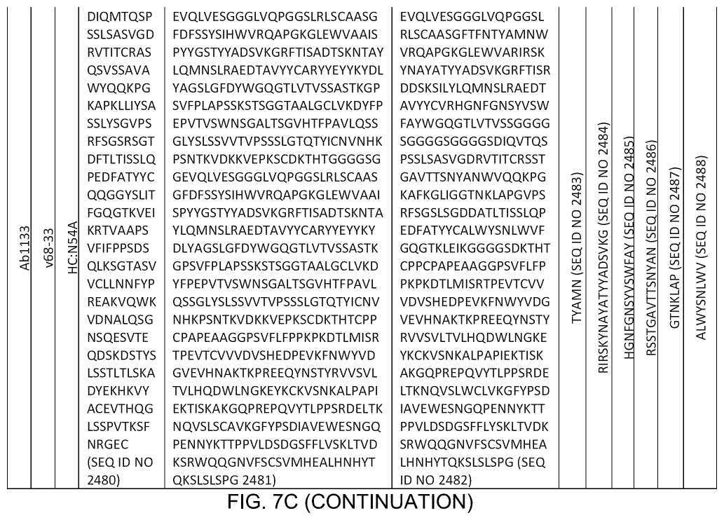

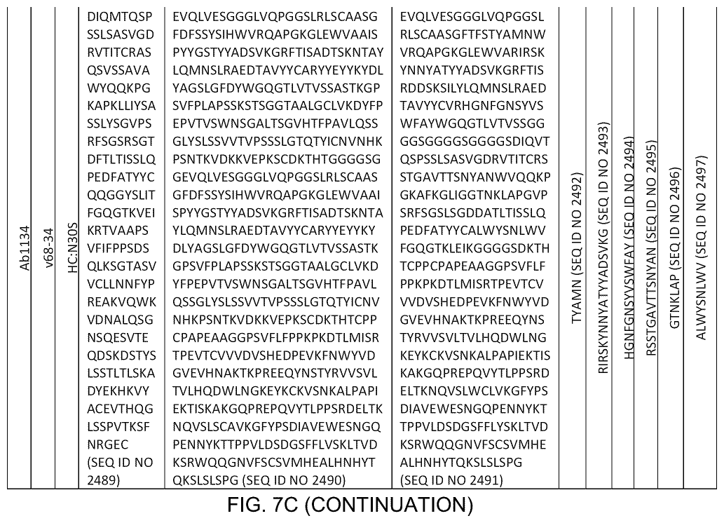

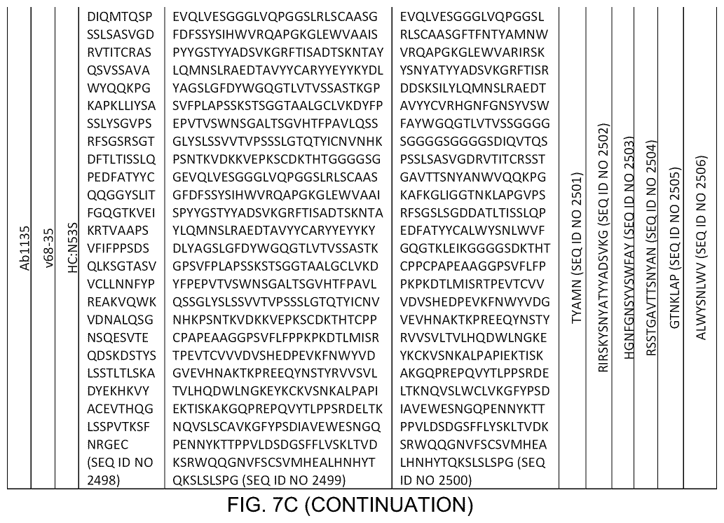

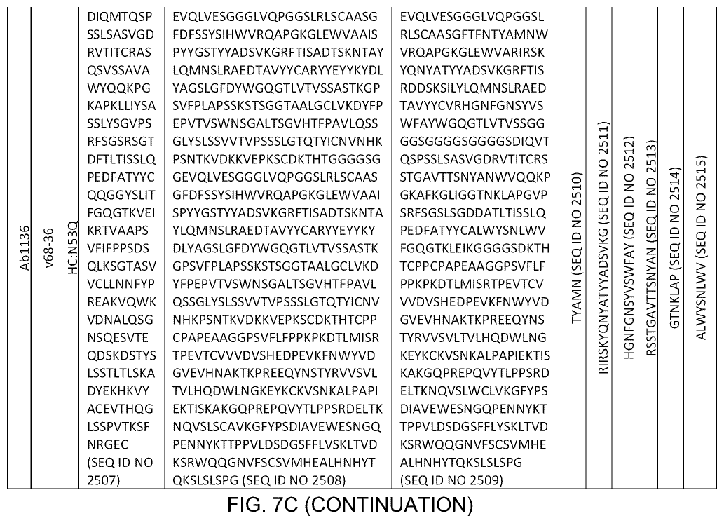

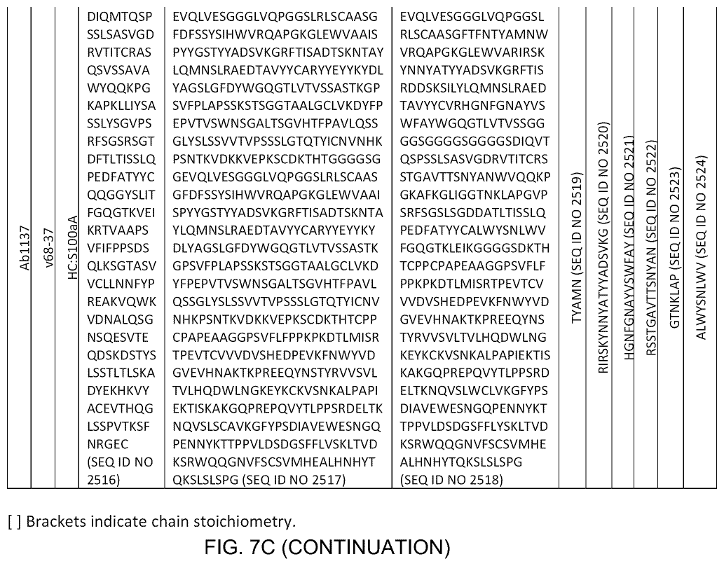

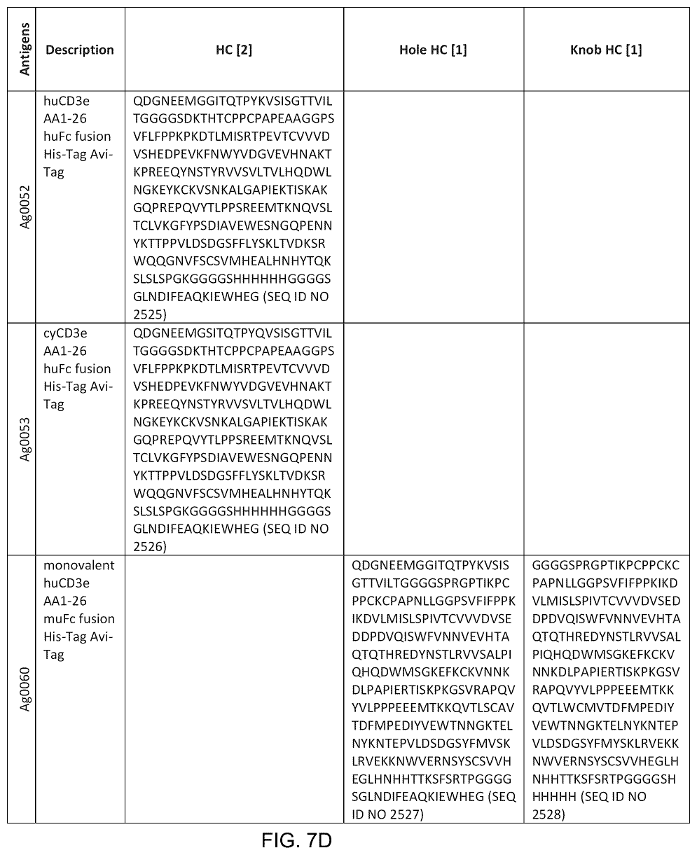

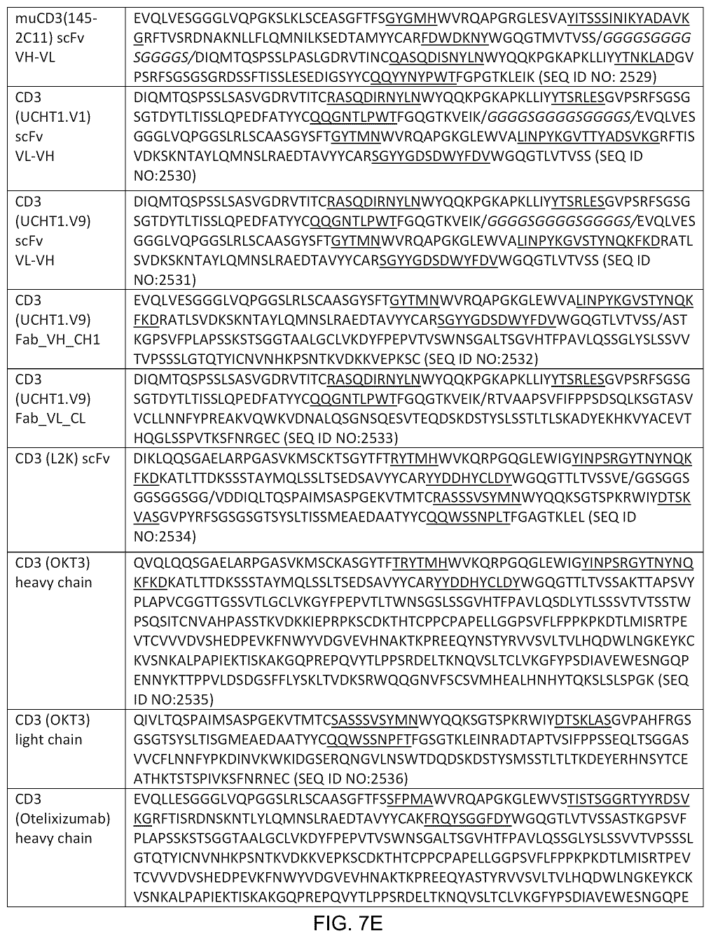

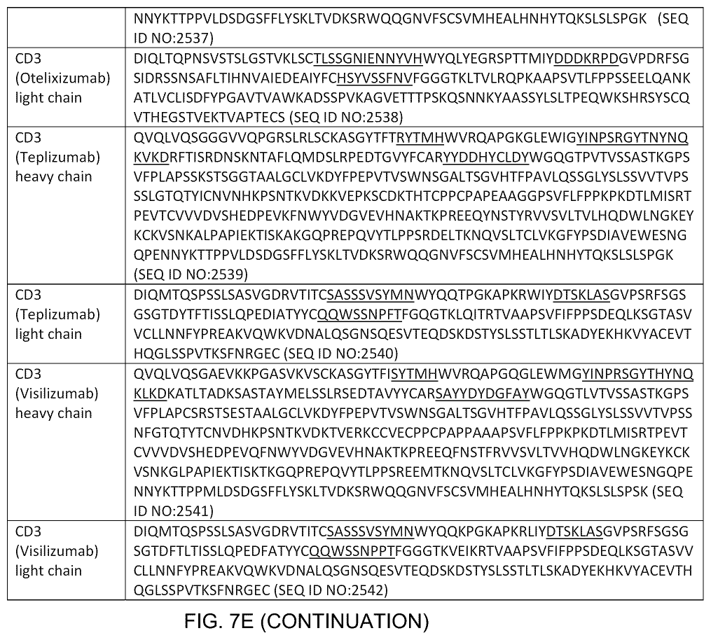

In some embodiments, the αCD3 ABDs comprise a set of three light chain CDRs (vlCDR1, vlCDR2 and vlCDR3), and three heavy chain CDRs (vhCDR1, vhCDR2 and vhCDR3) of an anti-CD3 antibody. Exemplary anti-CD3 antibodies contributing to the CDR sets, include, but are not limited to, L2K, UCHT1, variants of UCHT1 including UCHT1.v1 and UCHT1.v9, muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH12.5, F111-409, CLBT3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, and WT-31. Exemplary amino acid sequences of an αCD3 ABD or an αCD3 antibody are provided in FIGS. 7A-7C and 7E.

In some embodiments, the αCD3 ABD in this invention has from 0, 1, 2, 3, 4, 5 or 6 amino acid modifications based on the CDRs in the exemplary anti-CD3 antigen binding domains described herein (with amino acid substitutions finding particular use). That is, in some embodiments, the CDRs can be modified as long as the total number of changes in the set of 6 CDRs is less than 6 amino acid modifications, with any combination of CDRs being changed; e.g., there may be one amino acid change in vlCDR1, two in vhCDR2, none in vhCDR3, etc.

In some embodiments, the αCD3 ABD is humanized or from human. For example, the αCD3 ABD can comprise a light chain variable region comprising human CDRs or non-human light chain CDRs in a human light chain framework region; and a heavy chain variable region comprising human or non-human heavy chain CDRs in a human heavy chain framework region. In some embodiments, the light chain framework region is a lamda light chain framework. In other embodiments, the light chain framework region is a kappa light chain framework.

In some embodiments, the αCD3 ABD has an affinity to CD3 on CD3 expressing cells with a K_{D} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. The affinity to bind to CD3 can be determined, for example, by Surface Plasmon Resonance (SPR).

In one aspect, the composition of the present disclosure comprises a CD3 binding domain comprising a variable heavy (VH) domain comprising a vhCDR1, vhCDR2, and vhCDR3 sequence and a variable light (VL) domain comprising a vlCDR1, vlCDR2, and vlCDR3 sequence.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 7A-7C optionally with one or more mutations. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 7A-7C optionally with one or more mutations at positions corresponding to one or more of positions 30, 53, 54, and 100 in a heavy chain (HC) domain of Ab1091. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 7A-7C.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from Ab0640, Ab0769, Ab0770, Ab0771, Ab0772, Ab0773, Ab0774, Ab0775, Ab0776, Ab0907, Ab0908, Ab0911, Ab0912, Ab0913, Ab0914, Ab0915, Ab0916, Ab0917, Ab0918, Ab0919, Ab0920, Ab0921, Ab0922, Ab0923, Ab0924, Ab0925, Ab0926, Ab0927, Ab0928, Ab0929, Ab0930, Ab1091, Ab1133, Ab 1134, Ab 1135, Ab1136, and Ab1137 of FIGS. 7A-7C. In some embodiments, the sequences are from Ab0640. In some embodiments, the sequences are from Ab0769. In some embodiments, the sequences are from Ab0770. In some embodiments, the sequences are from Ab0771. In some embodiments, the sequences are from Ab0772. In some embodiments, the sequences are from Ab0773. In some embodiments, the sequences are from Ab0774. In some embodiments, the sequences are from Ab0775. In some embodiments, the sequences are from Ab0776. In some embodiments, the sequences are from Ab0907. In some embodiments, the sequences are from Ab0908. In some embodiments, the sequences are from Ab0911. In some embodiments, the sequences are from Ab0912. In some embodiments, the sequences are from Ab0913. In some embodiments, the sequences are from Ab0914. In some embodiments, the sequences are from Ab0915. In some embodiments, the sequences are from Ab0916. In some embodiments, the sequences are from Ab0917. In some embodiments, the sequences are from Ab0918. In some embodiments, the sequences are from Ab0919. In some embodiments, the sequences are from Ab0920. In some embodiments, the sequences are from Ab0921. In some embodiments, the sequences are from Ab0922. In some embodiments, the sequences are from Ab0923. In some embodiments, the sequences are from Ab0924. In some embodiments, the sequences are from Ab0925. In some embodiments, the sequences are from Ab0926. In some embodiments, the sequences are from Ab0927. In some embodiments, the sequences are from Ab0928. In some embodiments, the sequences are from Ab0929. In some embodiments, the sequences are from Ab0930. In some embodiments, the sequences are from Ab1091. In some embodiments, the sequences are from Ab1133. In some embodiments, the sequences are from Ab1134. In some embodiments, the sequences are from Ab1135. In some embodiments, the sequences are from Ab1136. In some embodiments, the sequences are from Ab1137.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1091 optionally with one or more mutations at positions 30, 53, 54, and 100 in the HC domain. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1091.

In some embodiments, the CD3 binding domain may comprise a VH domain and VL domain selected from the group consisting of the VH and VL domains from FIGS. 7A-7C optionally with one or more mutations. In some embodiments, the CD3 binding domain may comprise a VH domain and VL domain selected from the group consisting of the VH and VL domains from FIGS. 7A-7C.

In some embodiments, the CD3 binding domain may comprise a sequence having at least 85%, 86%, 87%, 88%, 89%, 90%, 91%, 92%, 93%, 94%, 95%, 96%, 97%, 98%, 99%, 100% sequence identity with the VH domain sequence of the Ab1091 and a sequence having at least 85%, 86%, 87%, 88%, 89%, 90%, 91%, 92%, 93%, 94%, 95%, 96%, 97%, 98%, 99%, 100% sequence identity with the VL domain sequence of the Ab1091. In some embodiments, the CD3 binding domain may comprise the VH and VL domain sequences of the Ab1091. In some embodiments, the CD3 binding domain may comprise the Ab1091.

In some embodiments, the vhCDR1, vhCDR2 and vhCDR3 sequences and said vlCDR1, vlCDR2, and vlCDR3 sequences may comprise one or more mutations selected from the group consisting of mutations corresponding to LC L54R, HC N53S+LC L54R+S56P, HC N53Q+LC L54R+S56P, HC N100S+LC L54R+S56P, HC S100aA+LC L54R+S56P, HC V100cA+LC L54R+S56P, HC S100dA+LC L54R+S56P, and LC L54R+S56P+W91Y in the HC domain of the Ab0640. In some embodiments, the vhCDR1, vhCDR2 and vhCDR3 sequences and said vlCDR1, vlCDR2, and vlCDR3 sequences may comprise one or more mutations selected from the group consisting of mutations corresponding to N30S, N53S, N53Q, N54A, and S100aA in the HC domain of the Ab1091. In some embodiments, the sequences have a mutation corresponding to said N30S. In some embodiments, the sequences have a mutation corresponding to said N53S. In some embodiments, the sequences have a mutation corresponding to said N53Q. In some embodiments, the sequences have a mutation corresponding to said N54A. In some embodiments, the sequences have a mutation corresponding to said S100aA.

In some embodiments, said composition comprises an Fc domain with one or more knob variants. In some embodiments, said composition comprises an Fc domain with one or more hole variants. Some examples of the knob and hole Fc sequences are disclosed in FIG. 10.

In some embodiments, the Fc domain described herein having a knob variant comprises a sequence having at least 85, 86, 87, 88, 89, 90, 91, 92, 93, 94, 95, 96, 97, 98, 99 and 100% identity to any one of SEQ ID NO: 3235, 3237, 3239, 3241, and 3243. In some embodiments, the Fc domain described herein having a hole variant comprises a sequence having at least 85, 86, 87, 88, 89, 90, 91, 92, 93, 94, 95, 96, 97, 98, 99 and 100% identity to any one of SEQ ID NO: 3236, 3238, 3240, 3242, and 3244. In some embodiments, the Fc domain described herein having a knob variant comprises the sequence of 3235. In some embodiments, the Fc domain described herein having a knob variant comprises the sequence of 3237. In some embodiments, the Fc domain described herein having a knob variant comprises the sequence of 3239. In some embodiments, the Fc domain described herein having a knob variant comprises the sequence of 3241. In some embodiments, the Fc domain described herein having a knob variant comprises the sequence of 3243. In some embodiments, the Fc domain described herein having a hole variant comprises the sequence of SEQ ID NO: 3236. In some embodiments, the Fc domain described herein having a hole variant comprises the sequence of SEQ ID NO: 3238. In some embodiments, the Fc domain described herein having a hole variant comprises the sequence of SEQ ID NO: 3240. In some embodiments, the Fc domain described herein having a hole variant comprises the sequence of SEQ ID NO: 3242. In some embodiments, the Fc domain described herein having a hole variant comprises the sequence of SEQ ID NO: 3244.

In some embodiments, the composition may comprise a CD3 binding domain comprising scFv means for binding CD3 to a scFv. In some embodiments, the composition may comprise CD3 binding domain comprising Fab means for binding CD3. In some embodiments, the composition may be a nucleic acid composition comprising a polynucleotide encoding a composition comprising an indinavir binding domain. In some embodiments, an expression vector composition may comprise the nucleic acid composition. In some embodiments, a host cell may comprise the expression vector.

### 5. Anti-Tumor Targeting Antigen Binding Domains (αTTABDs)

All three formats of the invention rely on the use of targeting to tumors, and thus all formats utilize one or more anti-tumor targeting antigen binding domains (αTTABDs).

CT binding proteins described in this invention comprises one or more anti-tumor targeting antigen binding domains (αTTABDs). In some embodiments, the αTTABD binds to a target antigen involved in and/or associated with a tumorous disease, disorder or condition. In some embodiments, the αTTABD binds to a tumor-associated antigen, which is a cell surface molecule such as a protein, lipid or polysaccharide. In some embodiments, the αTTABD binds to a tumor-associated antigen expressed on a tumor cell or tumor microenvironment.

CTCoS binding proteins described in this invention comprises one or more anti-tumor targeting antigen binding domains (αTTABDs). In some embodiments, the αTTABD binds to a target antigen involved in and/or associated with a tumorous disease, disorder or condition. In some embodiments, the αTTABD binds to a tumor-associated antigen, which is a cell surface molecule such as a protein, lipid or polysaccharide. In some embodiments, the αTTABD binds to a tumor-associated antigen expressed on a tumor cell or tumor microenvironment.

CTTCoS binding proteins described in this invention comprises two or more anti-tumor targeting antigen binding domains (αTTABDs). In some embodiments, the αTTABDs bind to target antigens involved in and/or associated with a tumorous disease, disorder or condition. In some embodiments, the αTTABDs bind to a tumor-associated antigen, which is a cell surface molecule such as a protein, lipid or polysaccharide. In some embodiments, the αTTABDs bind to a tumor-associated antigen expressed on a tumor cell or tumor microenvironment. In some embodiments, the two or more αTTABDs in the CTCoS binding proteins bind to the same tumor-associated antigen. In some embodiments, the two or more αTTABDs in the CTCoS binding proteins bind to different tumor-associated antigens.

The αTTABDs in this invention can take any format, including but not limited to a full antibody, a Fab, an Fv, a single chain variable fragments (scFv), a scFab, a single domain antibody such as the VHH of camelid derived single domain antibody.

In some embodiments, the αTTABDs bind to a tumor-associated antigen expressed on tumor cells. For example, the tumor-associated antigen can be CD19, and the BrighT-LITE incorporating an α-CD19 antigen binding domain (ABD) can be used to target CD19 expressing tumors, such as most B cell malignancies including but not limited to acute lymphoblastic leukemia (ALL), chronic lymphocytic leukemia (CLL) and B cell lymphomas. Exemplary α-CD19 ABDs can include one or more CDRs derived from the anti-CD19 binding domain of Blinatumomab, SAR3419, MEDI-551, or Combotox. In some other embodiments, α-CD19 ABDs can include one or more CDRs derived from an anti-CD19 antibody, such as clone FMC63 or clone HD37.

Other tumor-associated antigens include but are not limited to EpCAM, HER2, and CD20. In some embodiments, said other tumor-associated antigen is EpCAM.

In one aspect, a composition of the present disclosure comprises an EpCAM binding domain comprising, a) a variable heavy (VH) domain comprising a vhCDR1, vhCDR2, and vhCDR3 sequence; b) a variable light (VL) domain comprising a vlCDR1, vlCDR2, and vlCDR3 sequence.

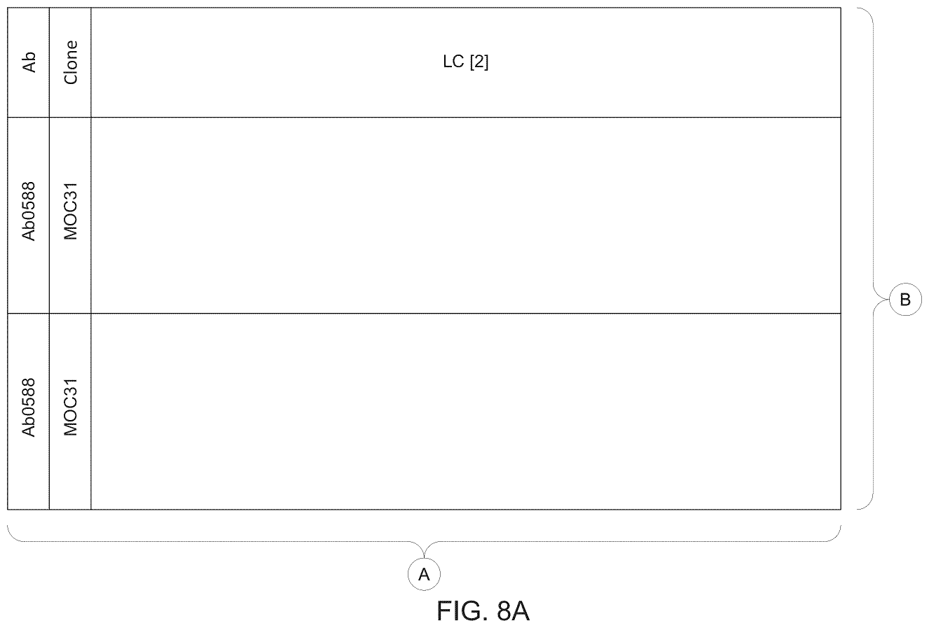

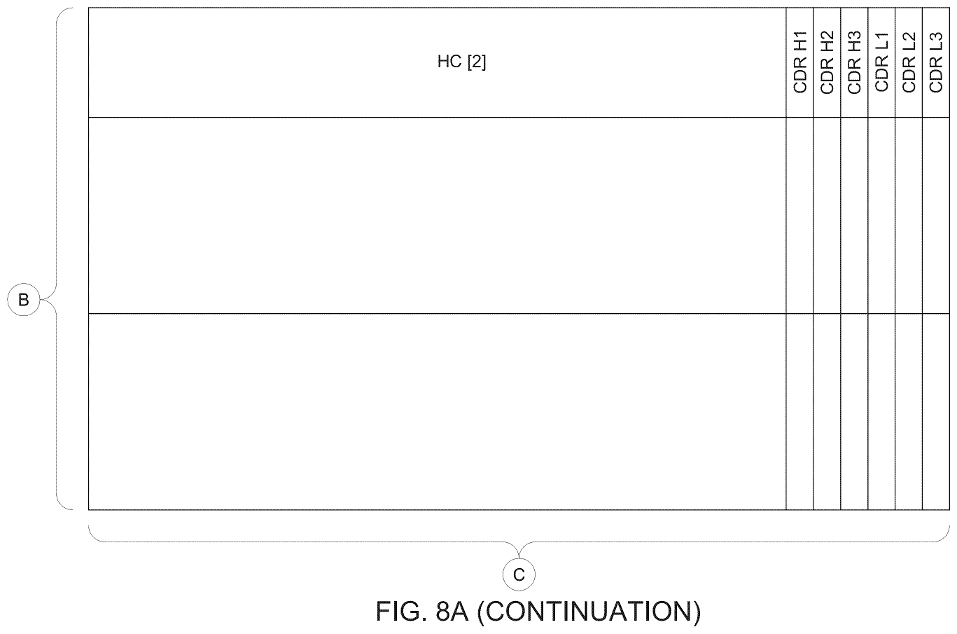

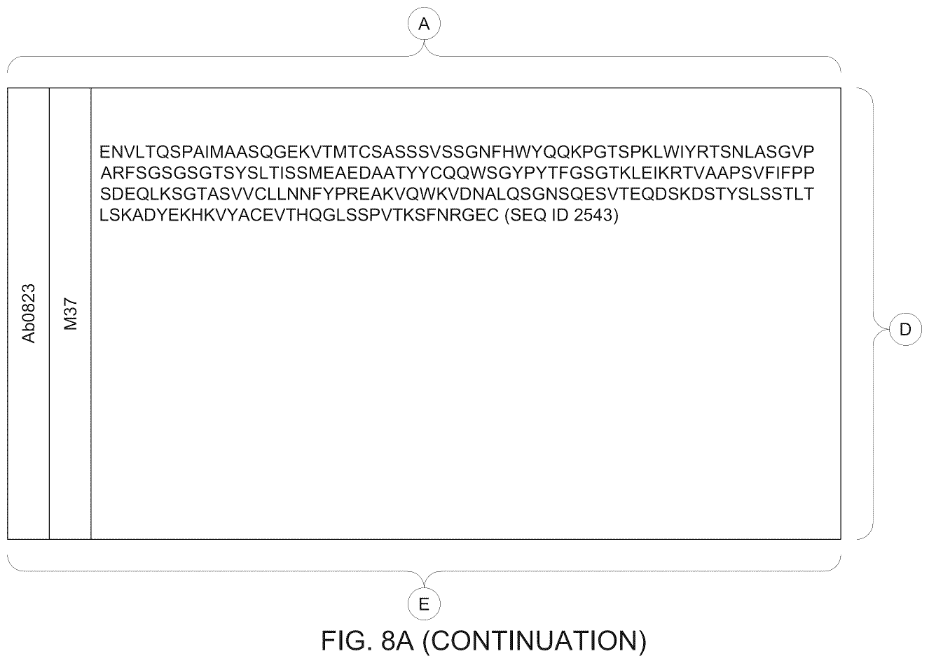

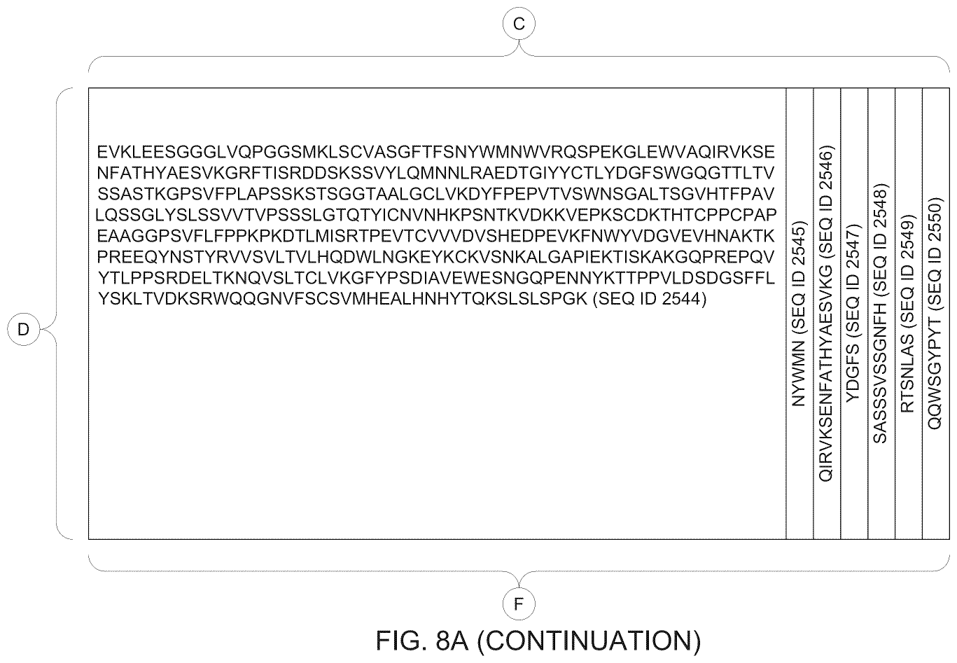

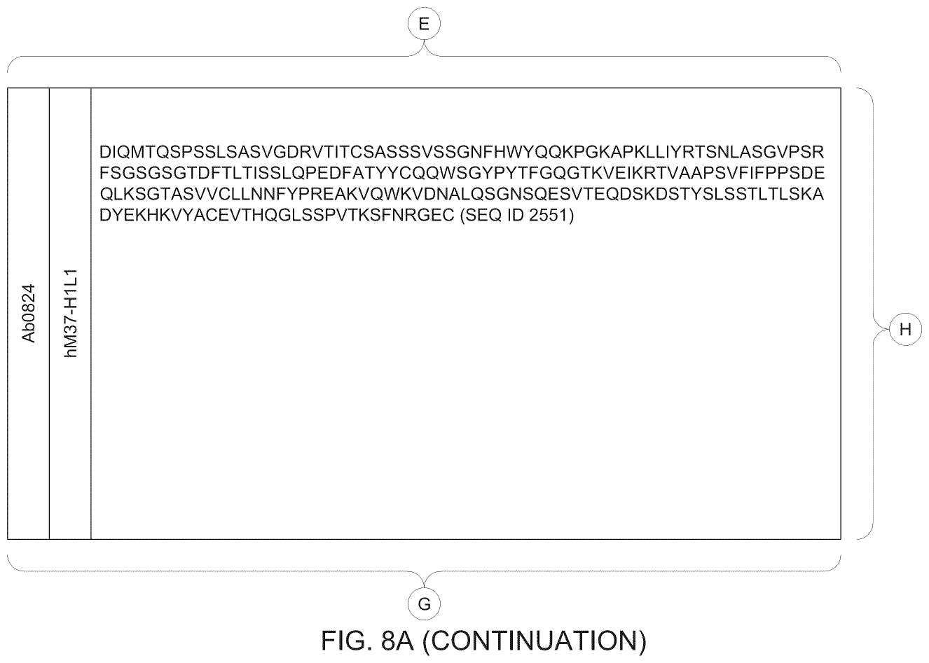

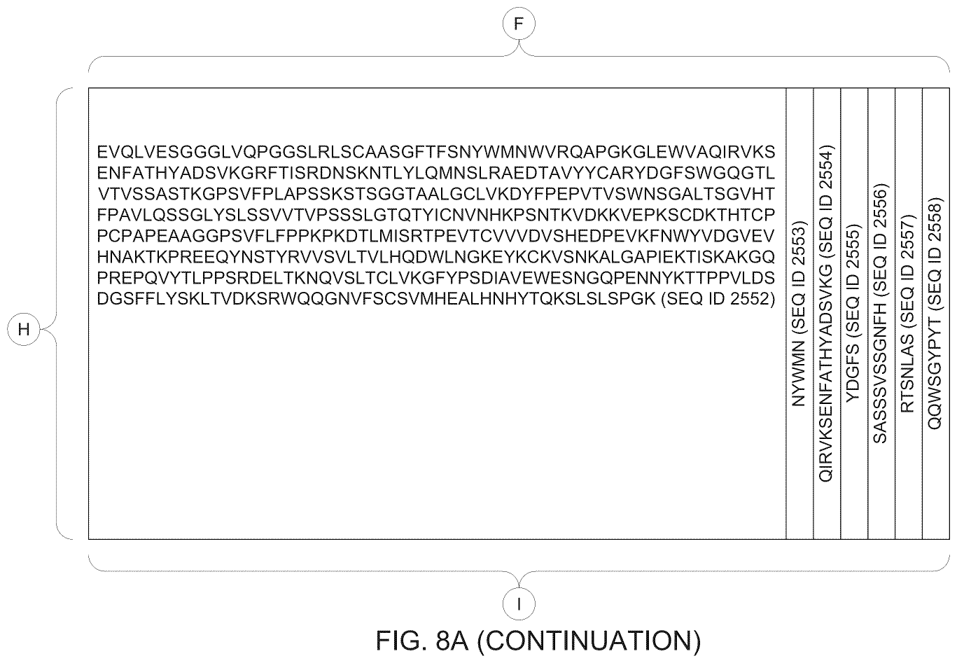

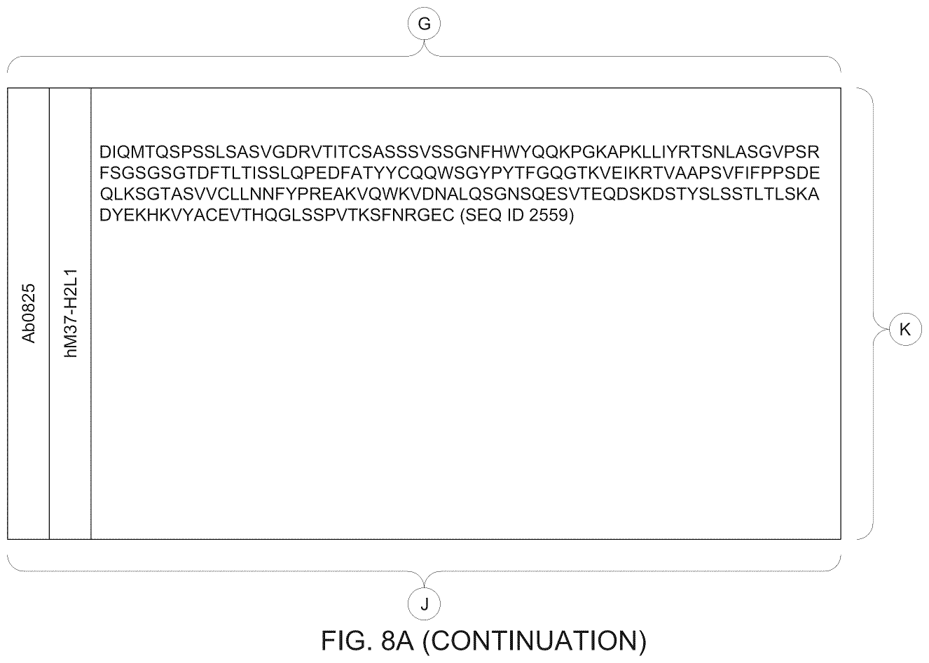

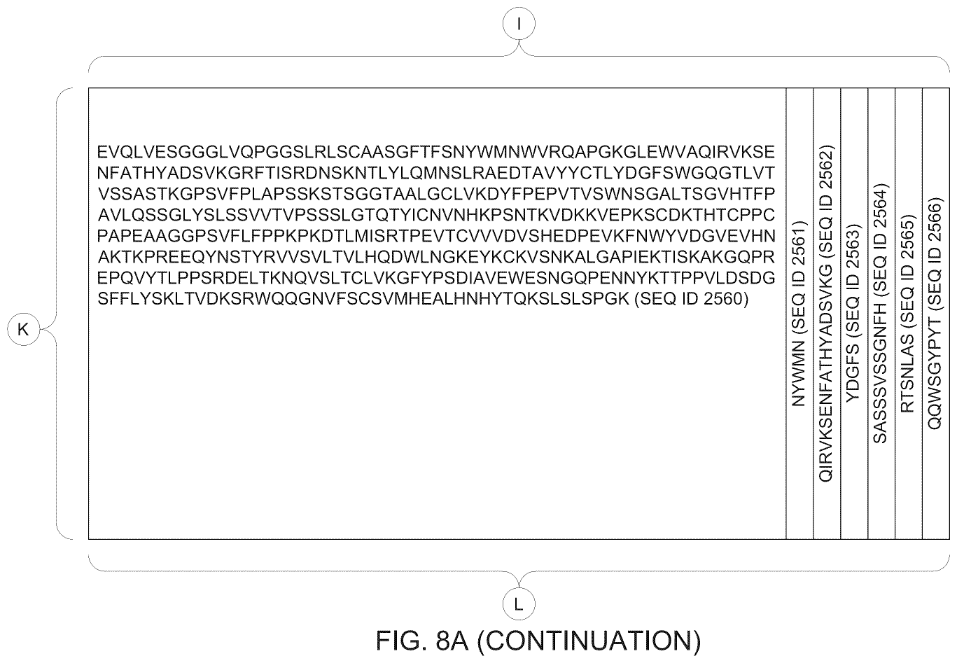

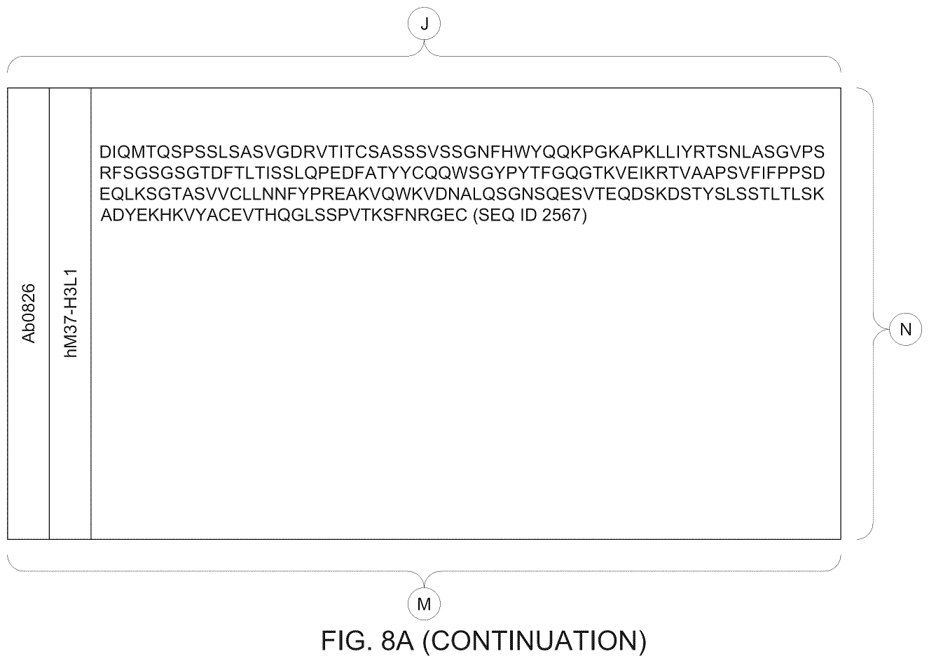

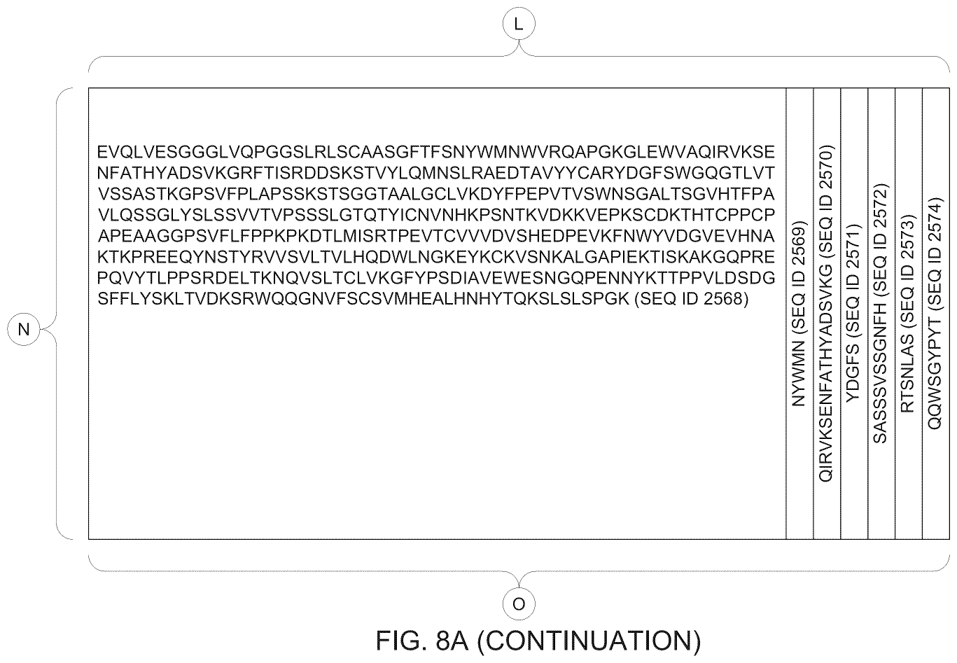

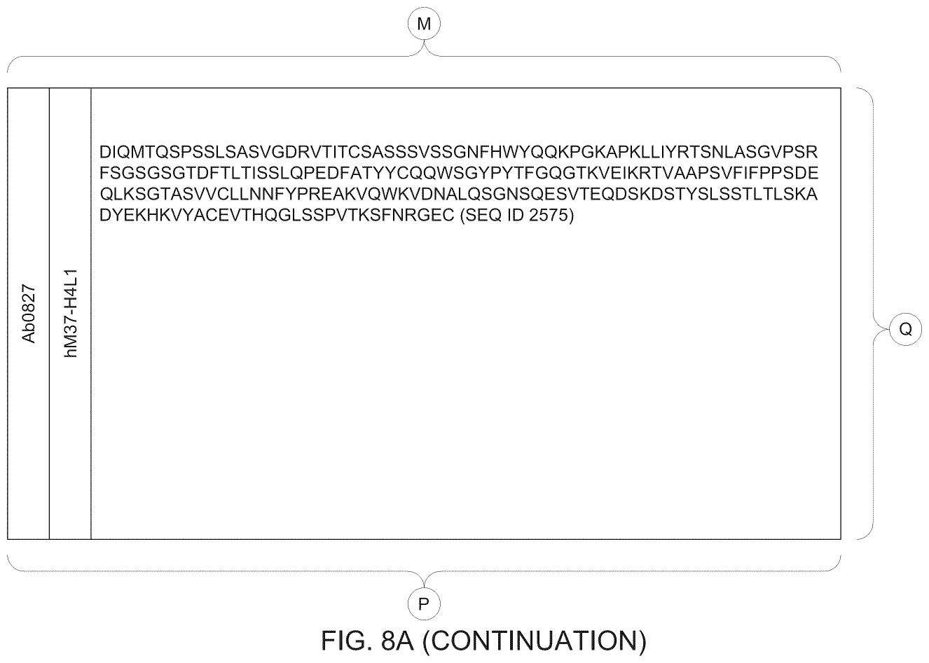

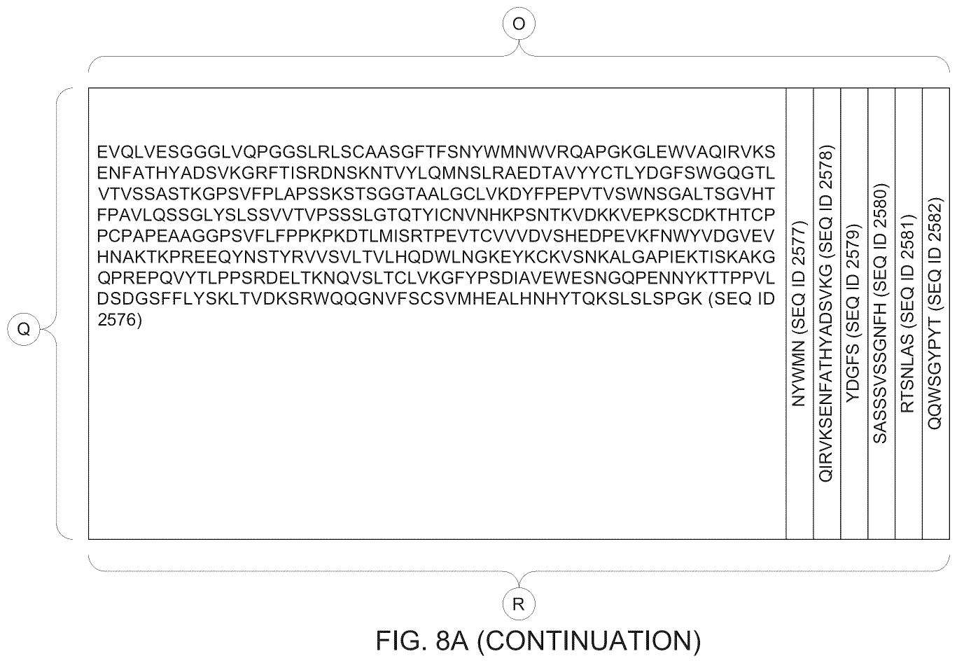

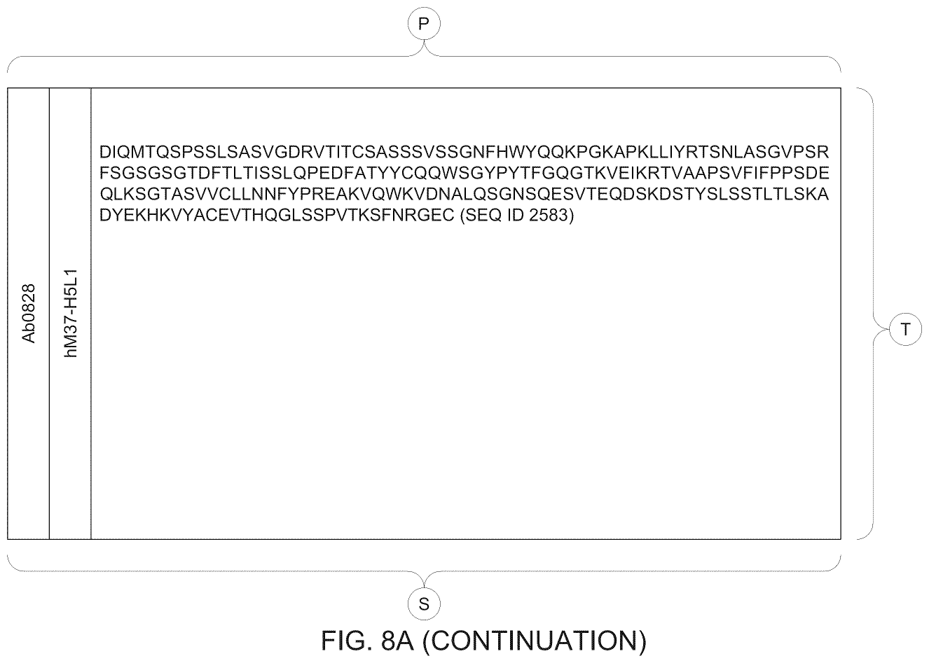

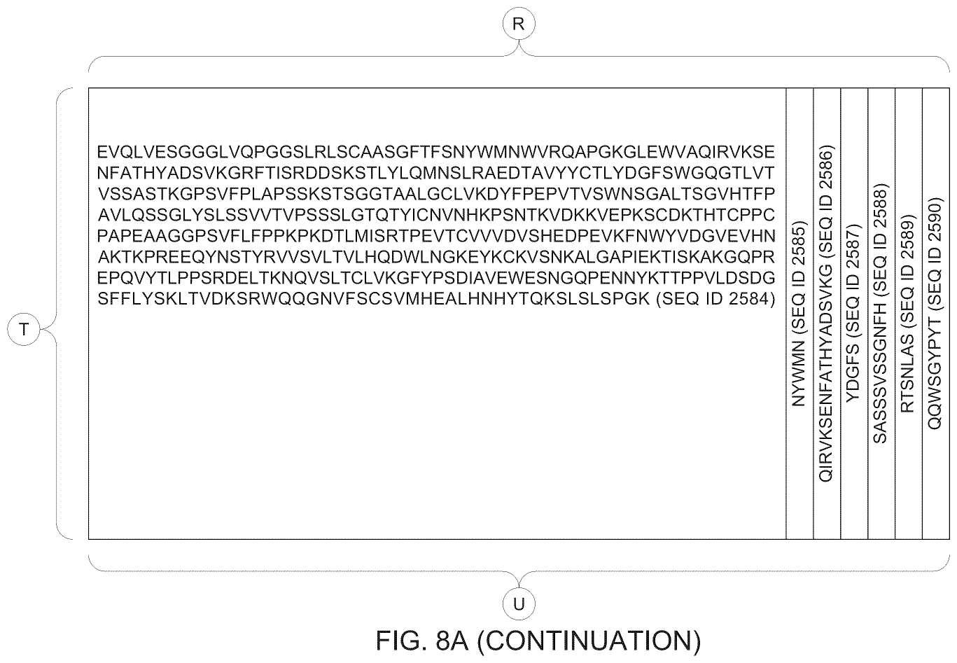

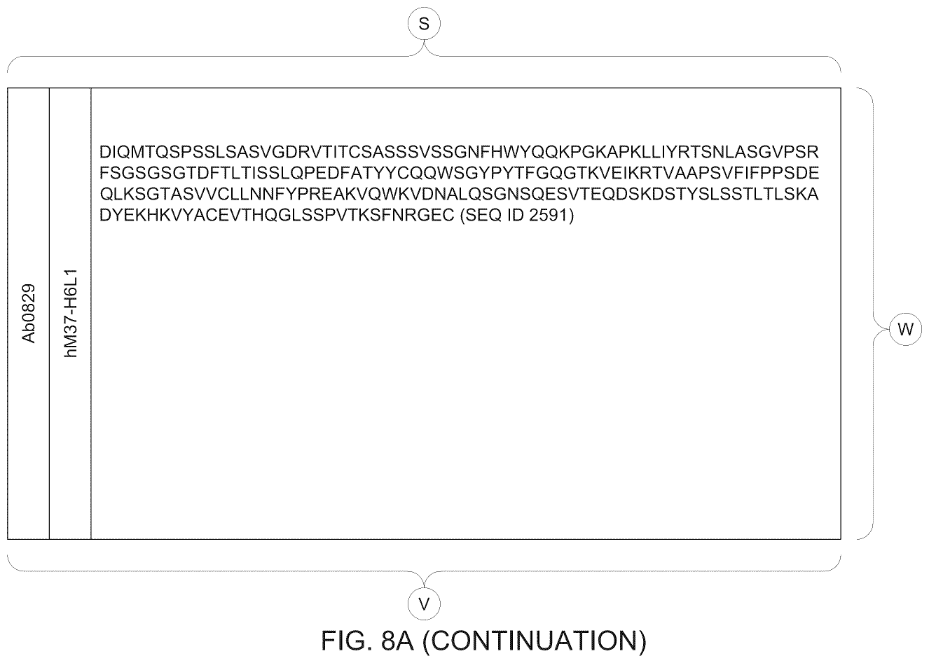

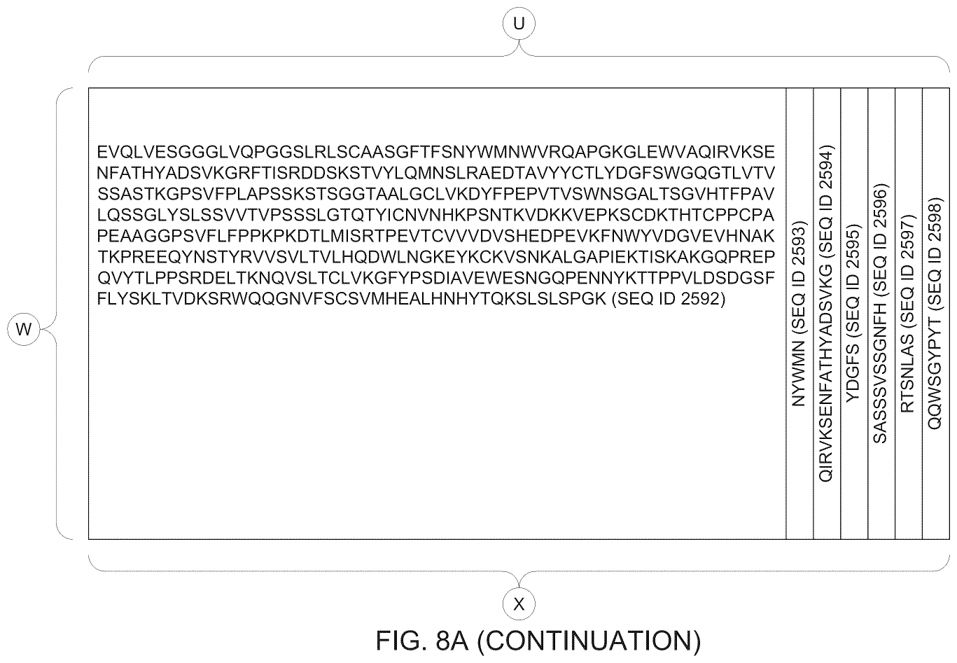

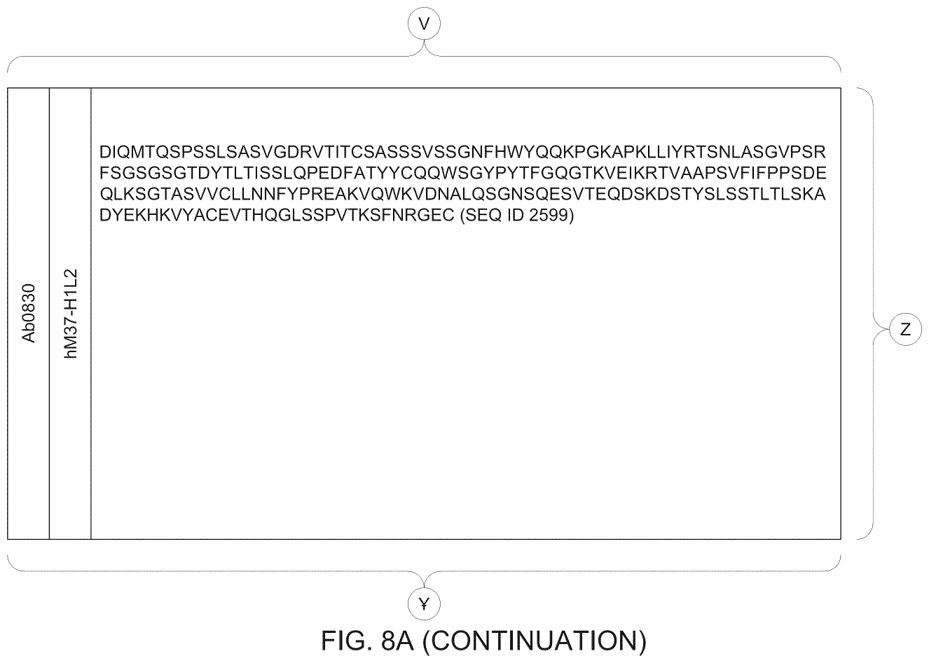

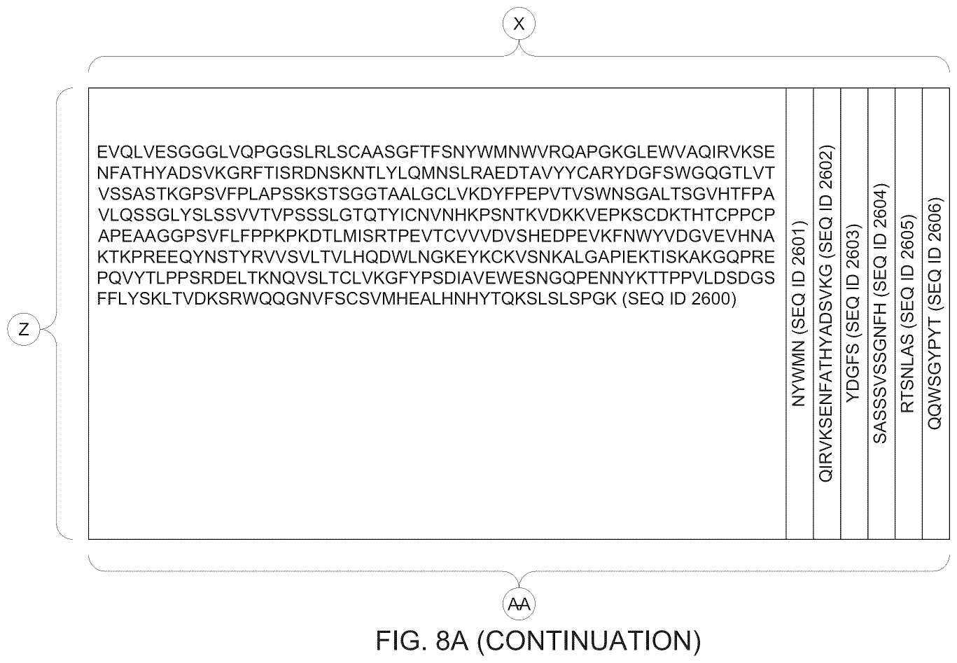

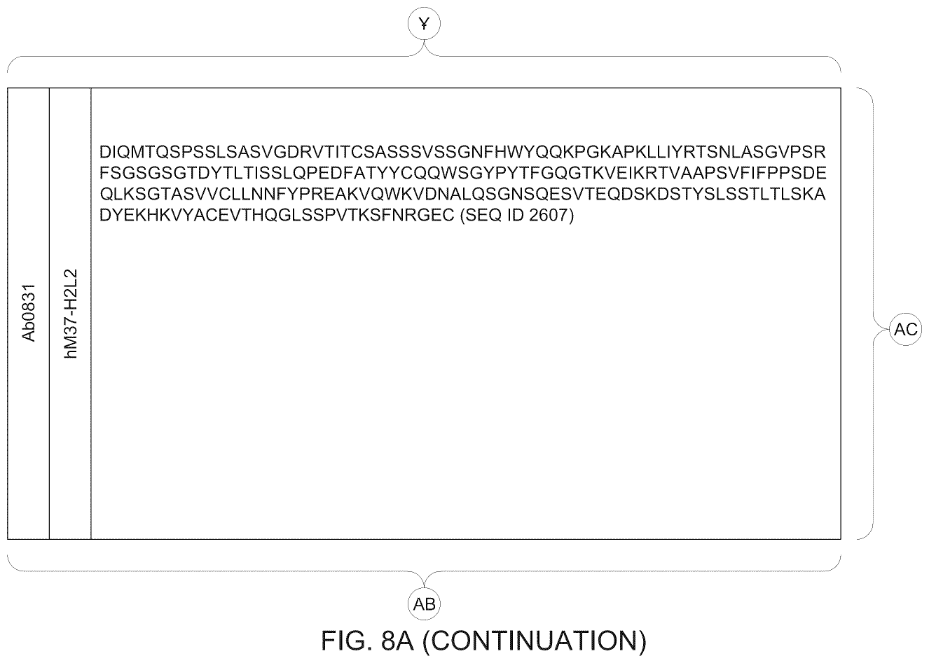

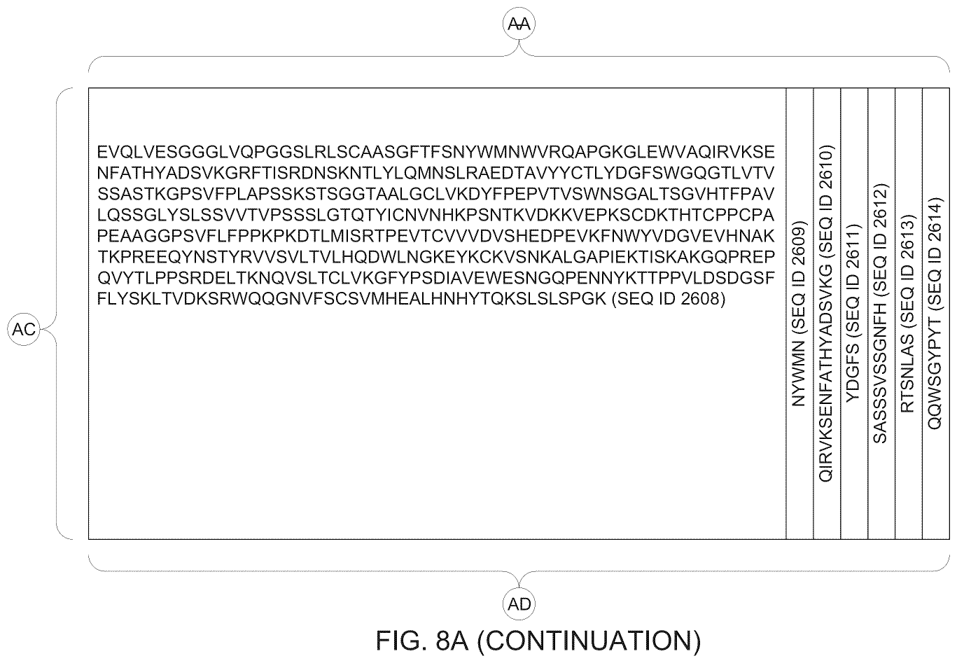

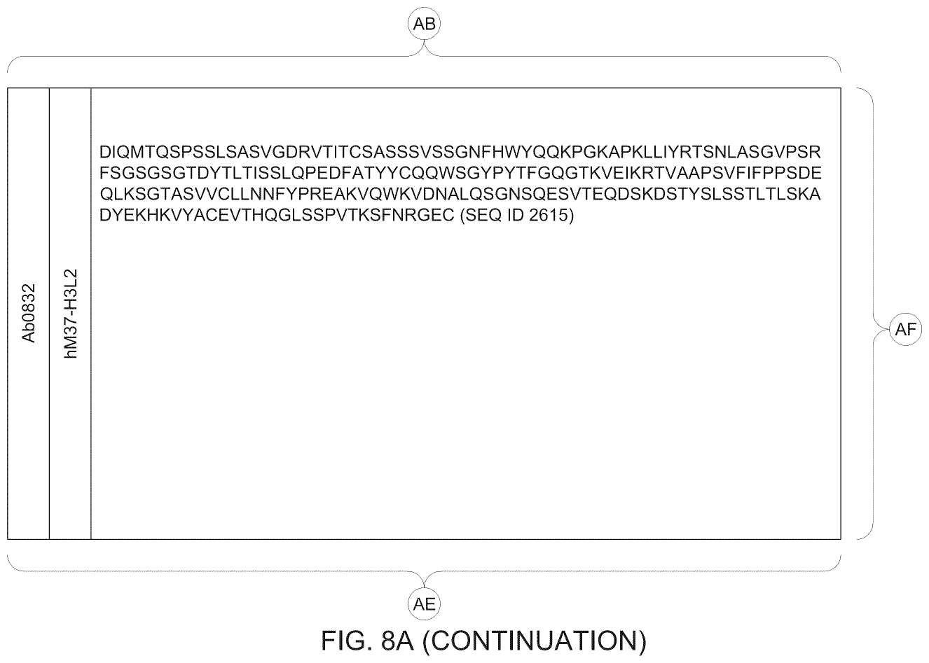

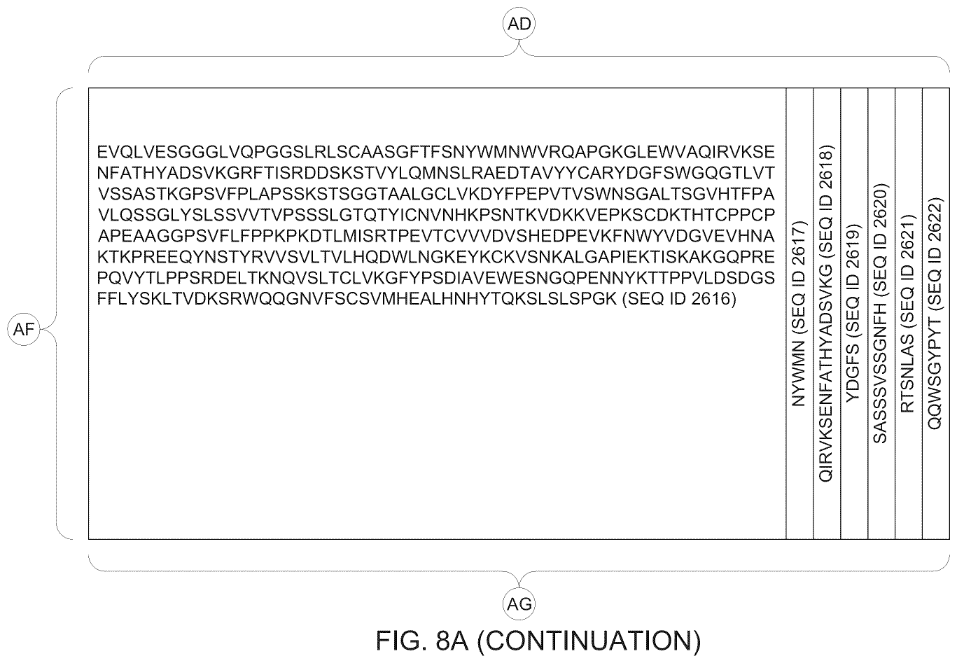

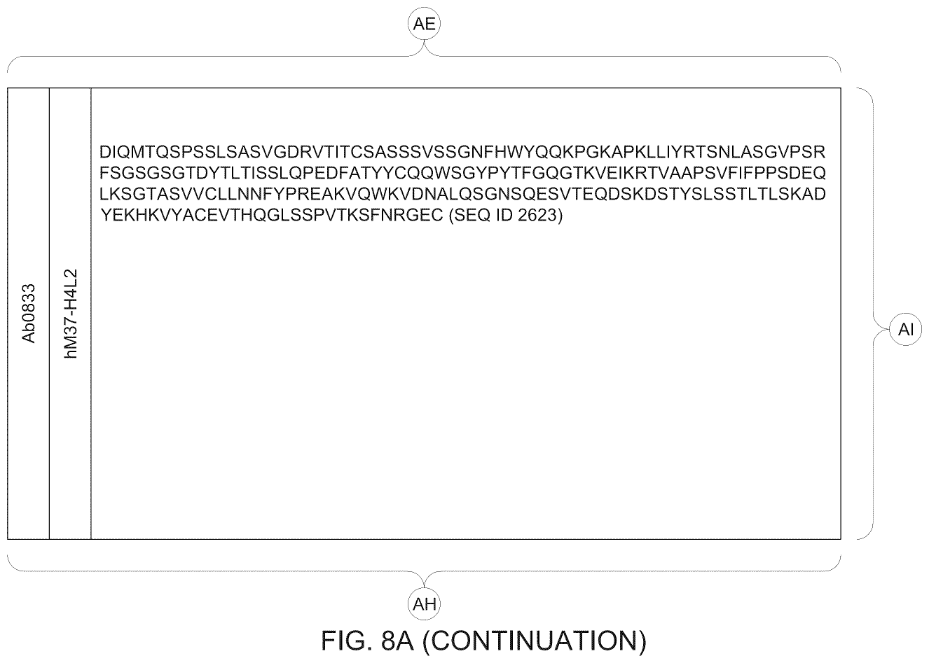

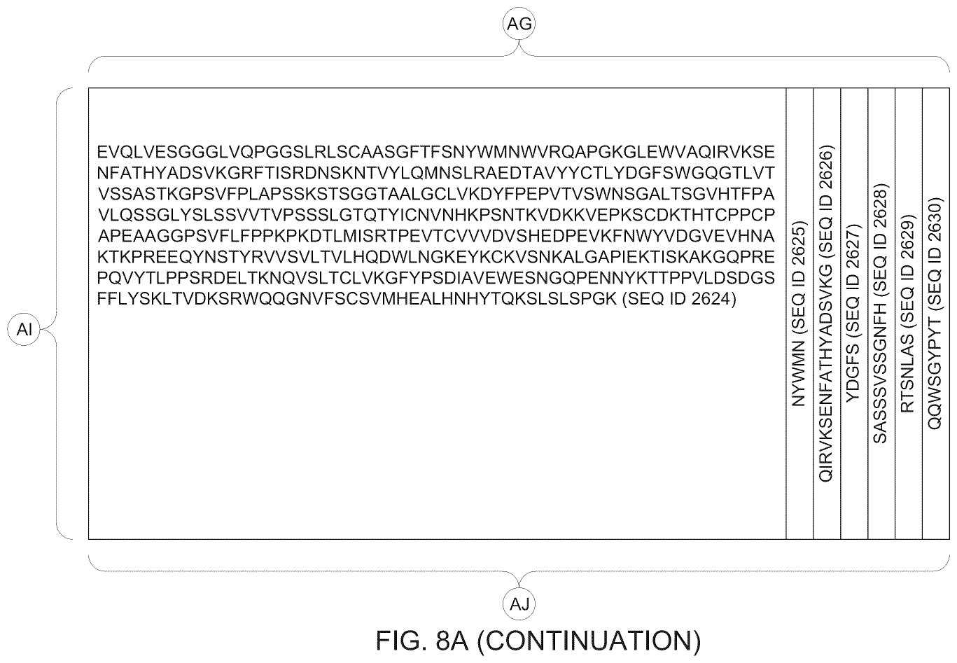

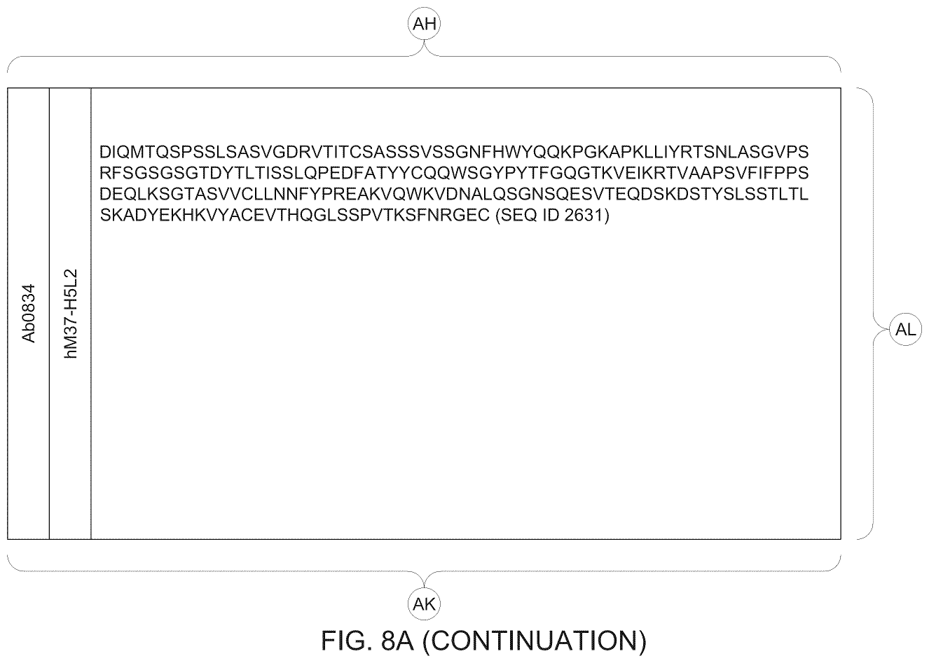

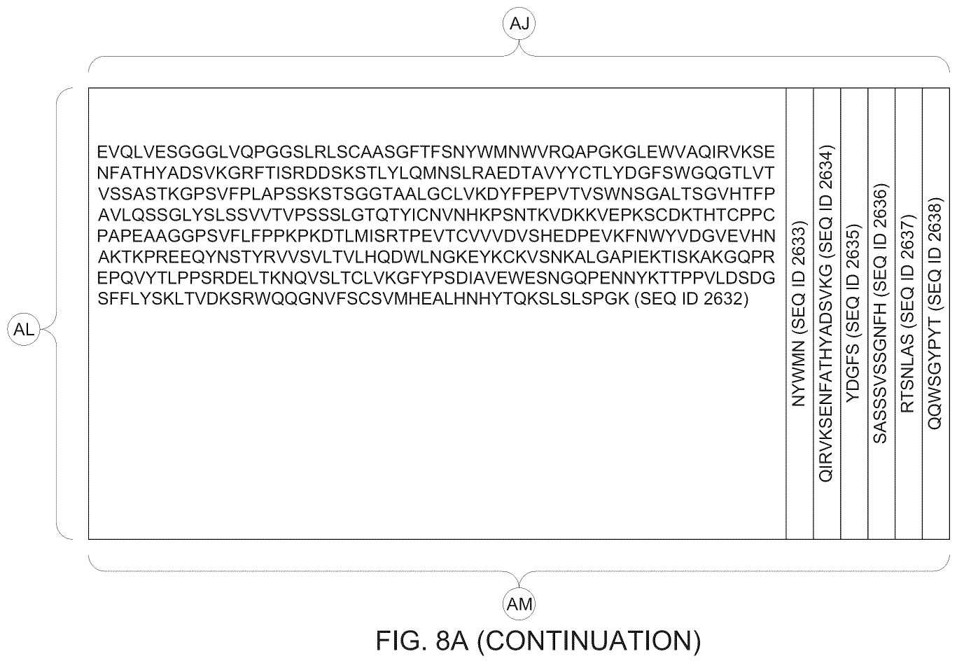

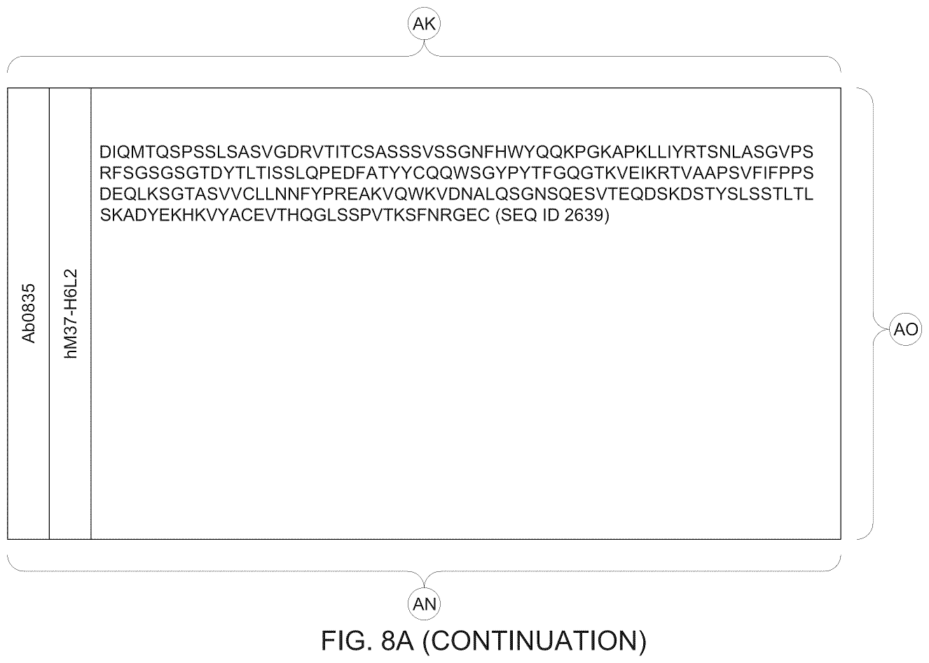

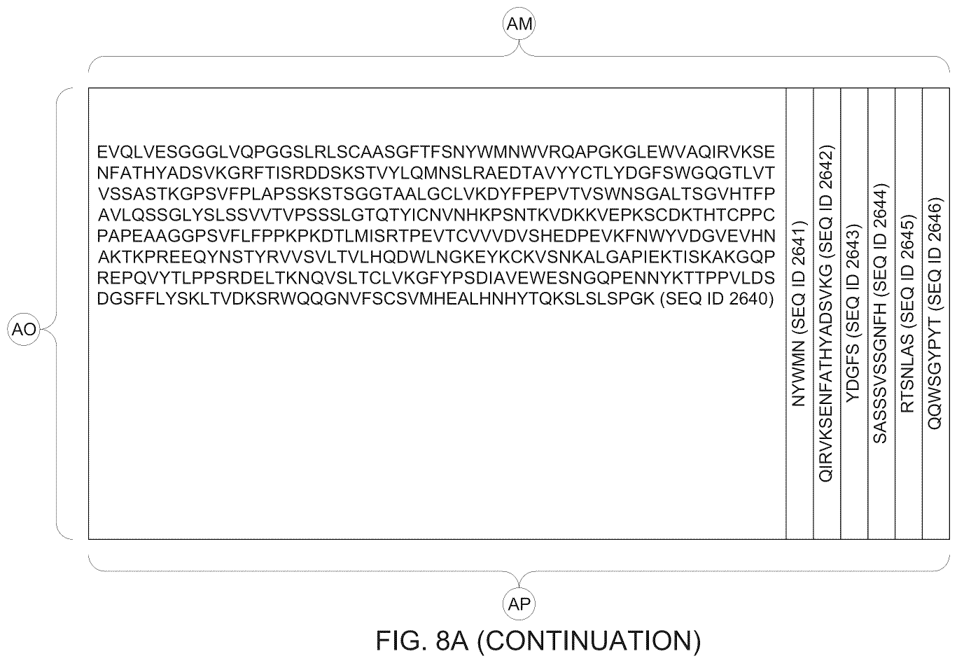

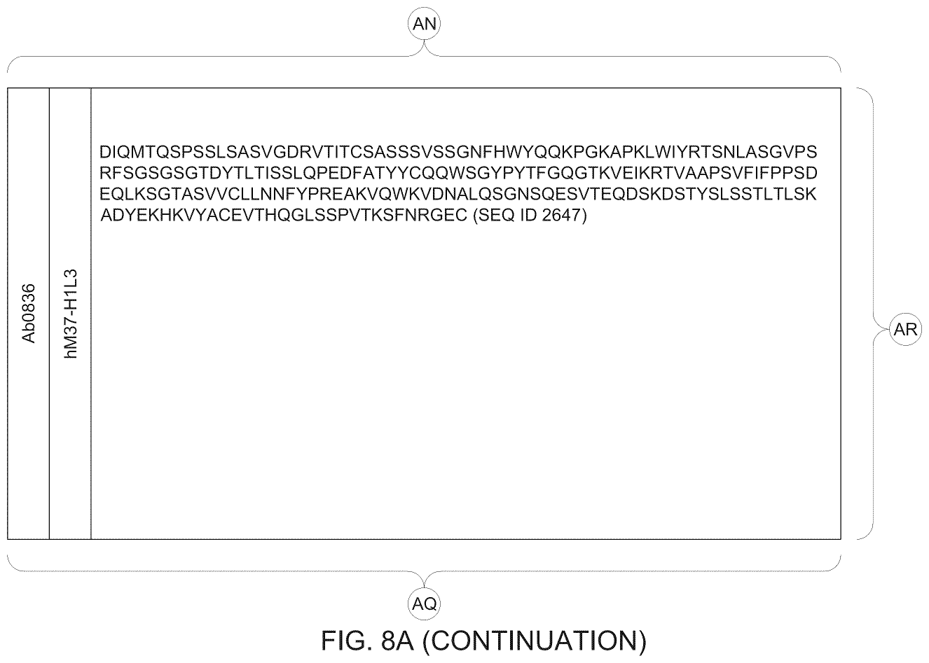

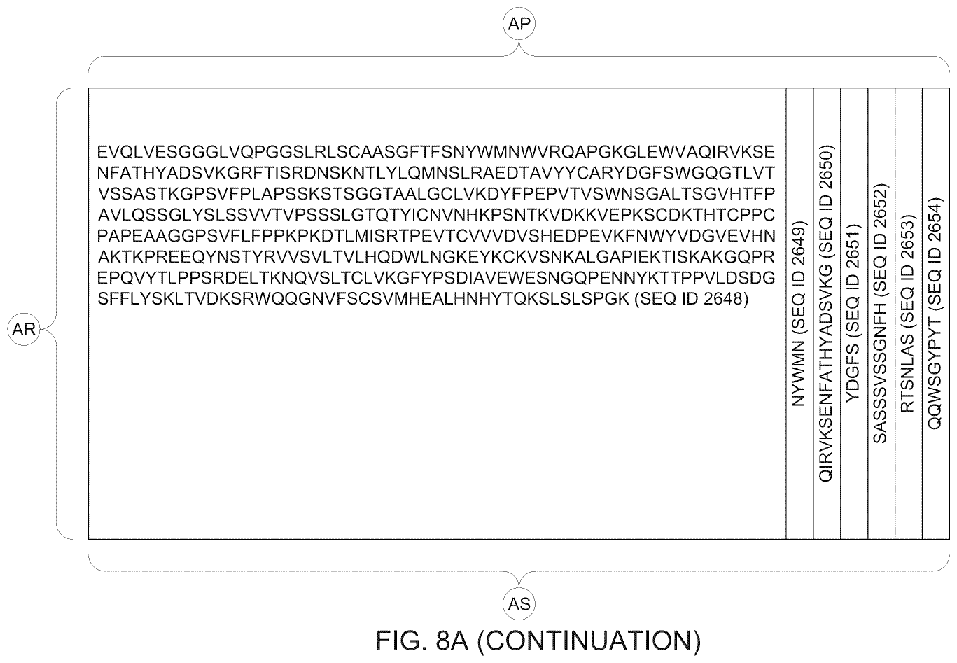

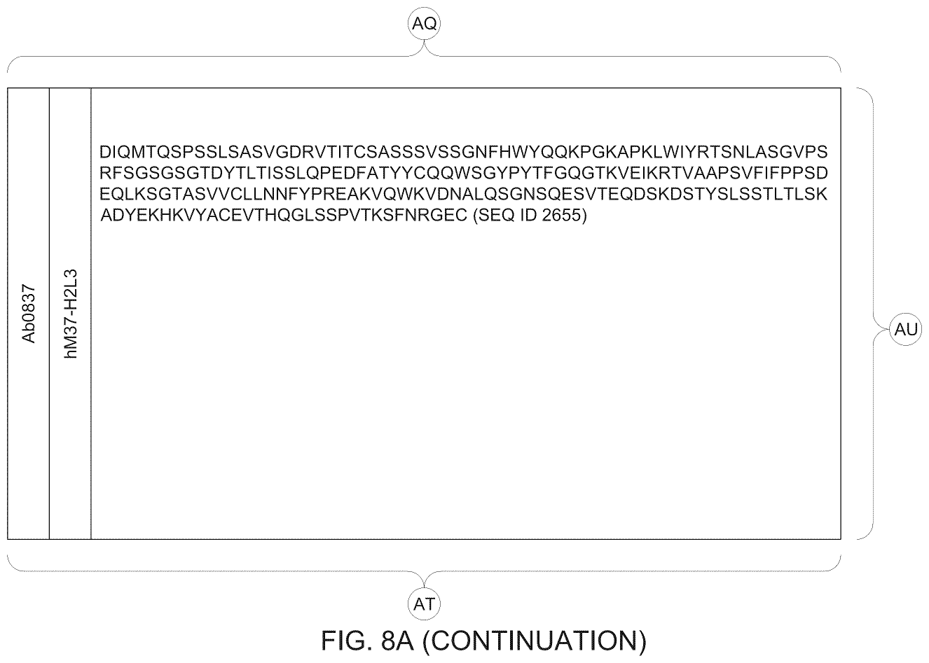

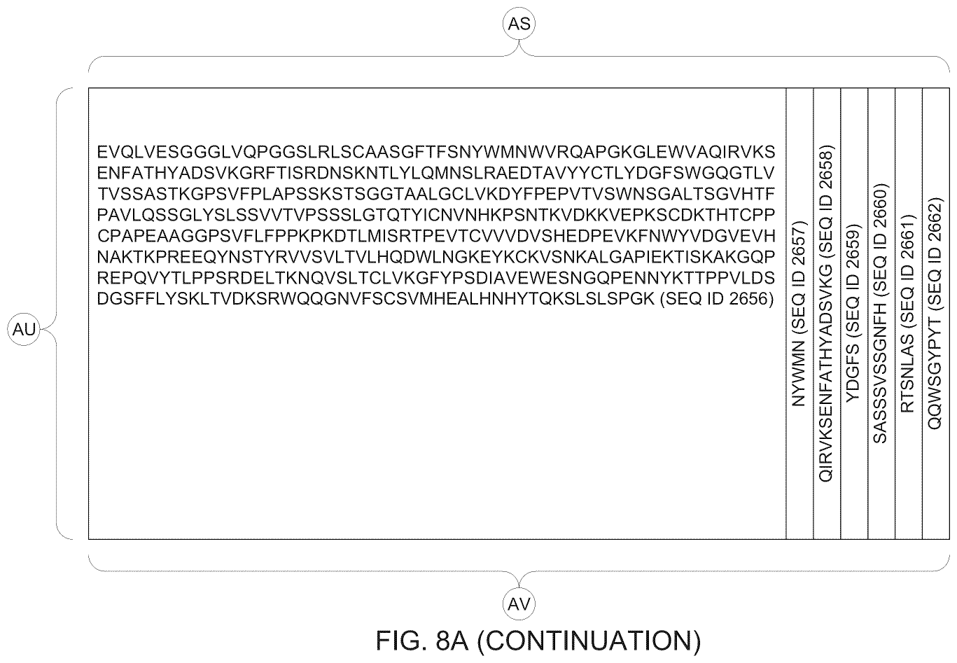

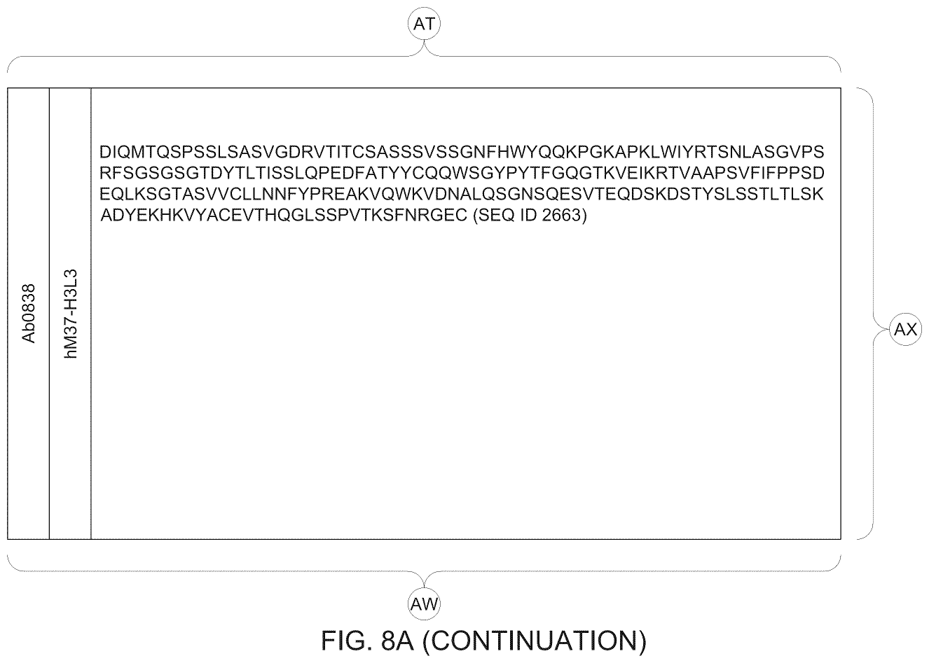

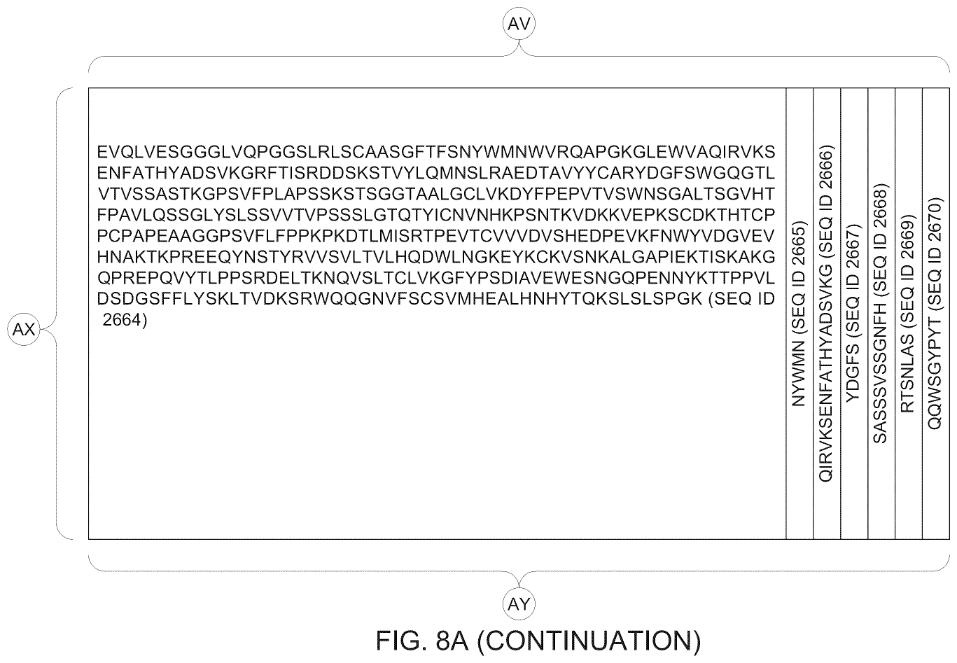

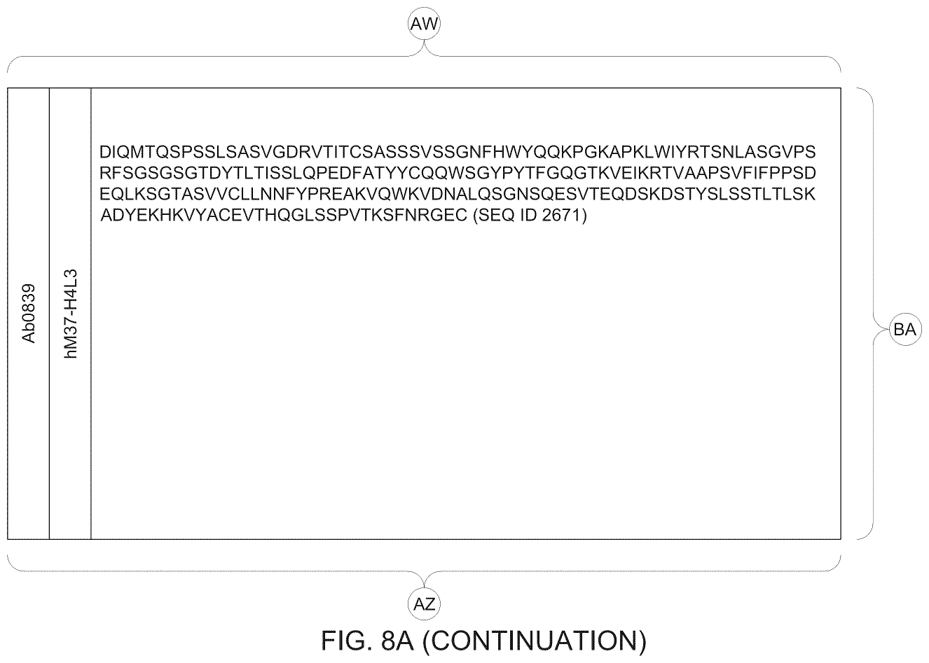

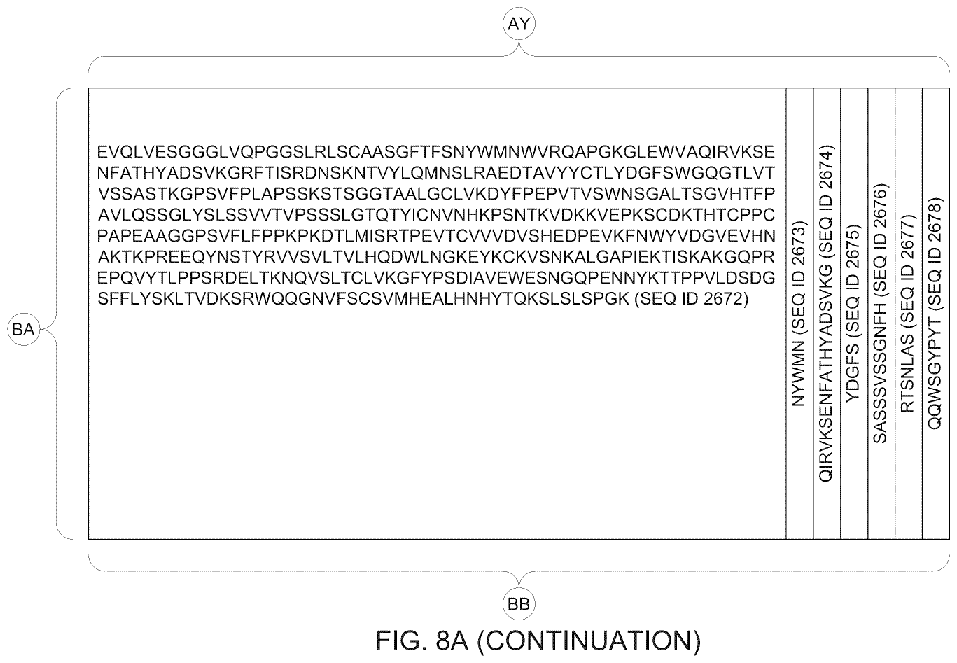

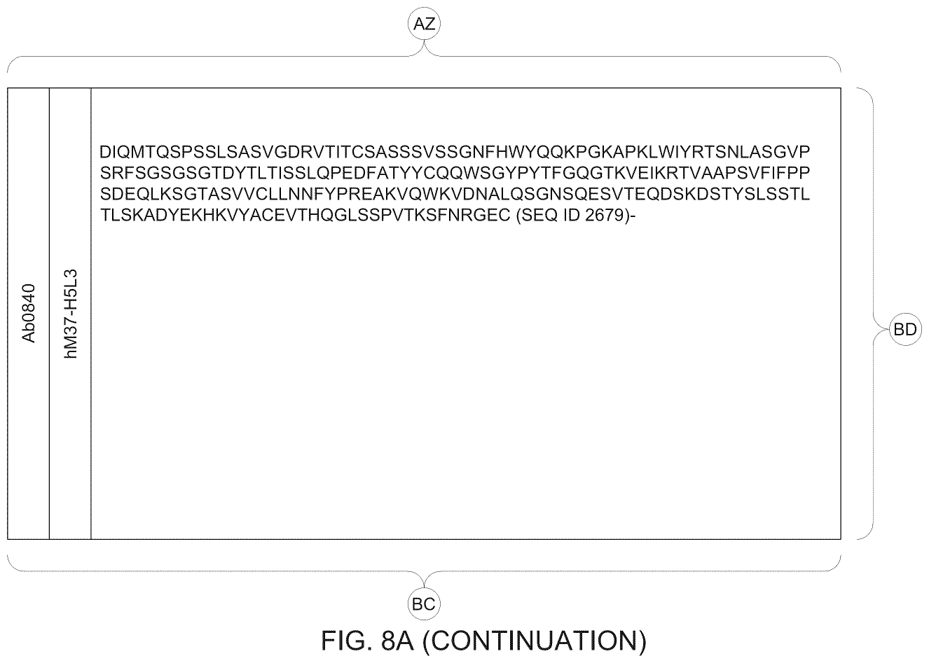

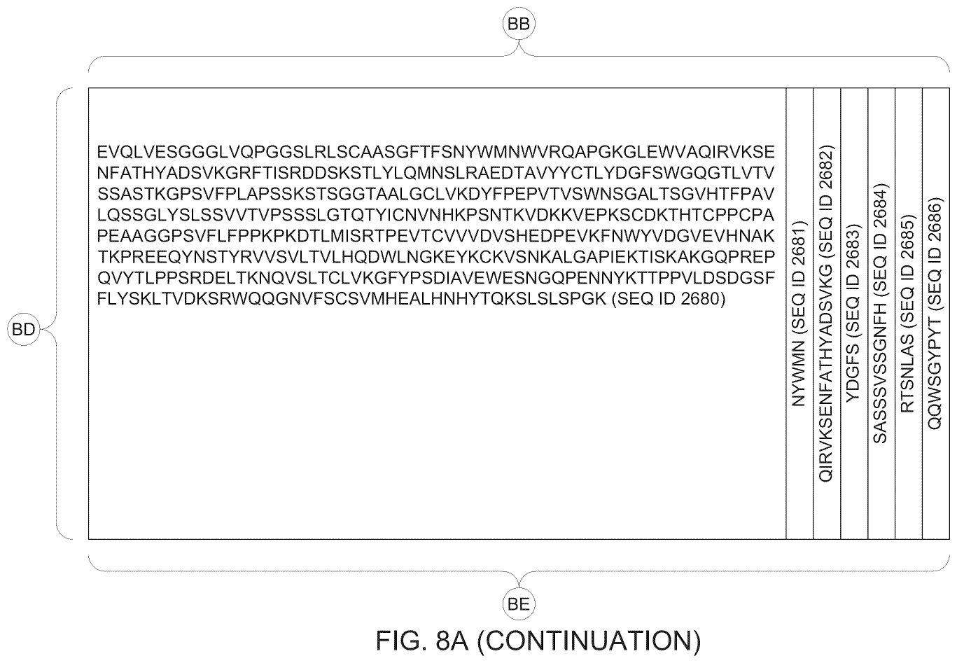

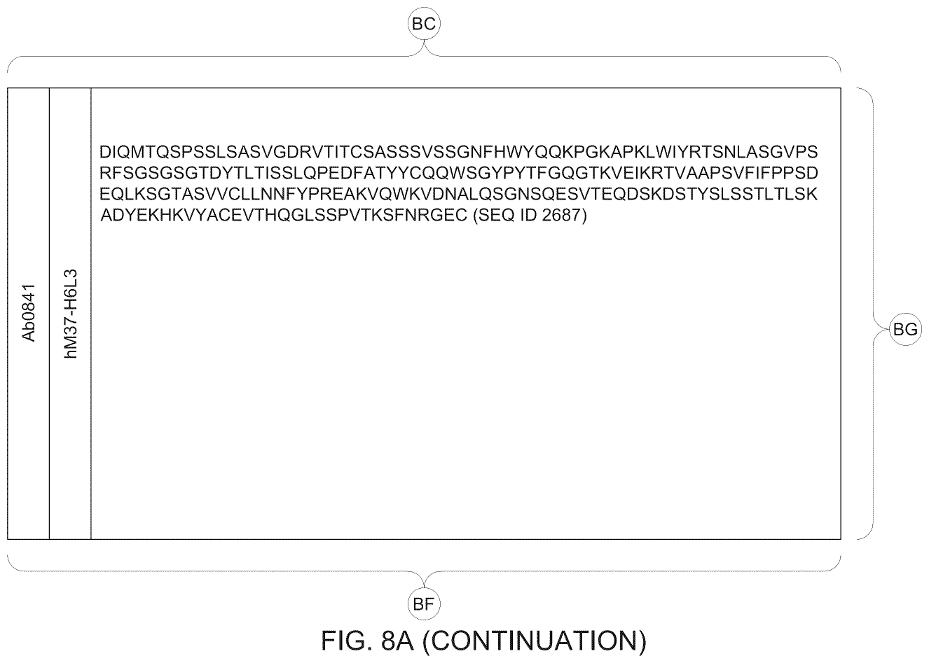

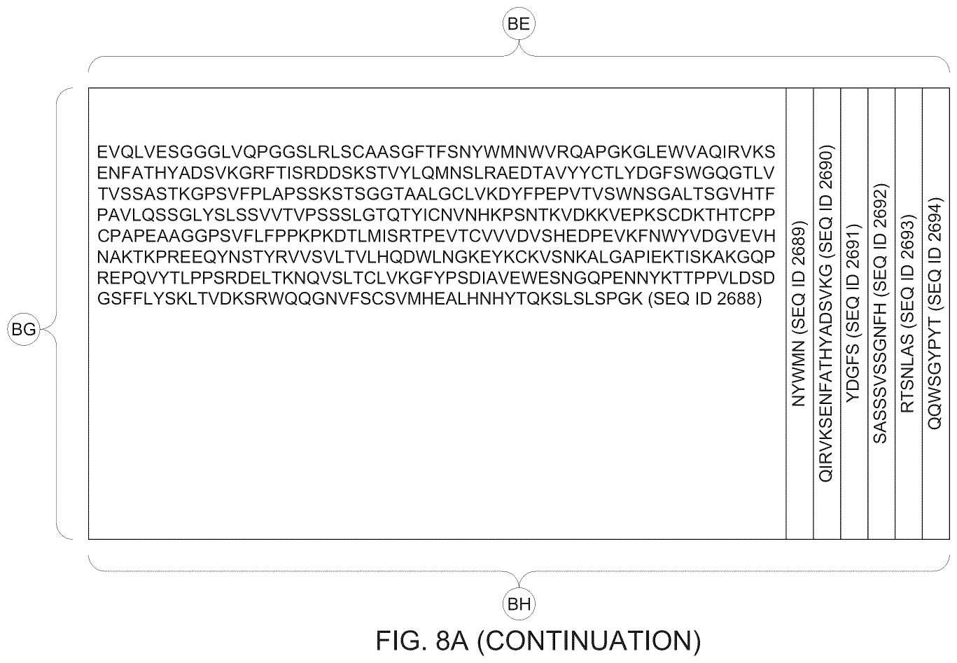

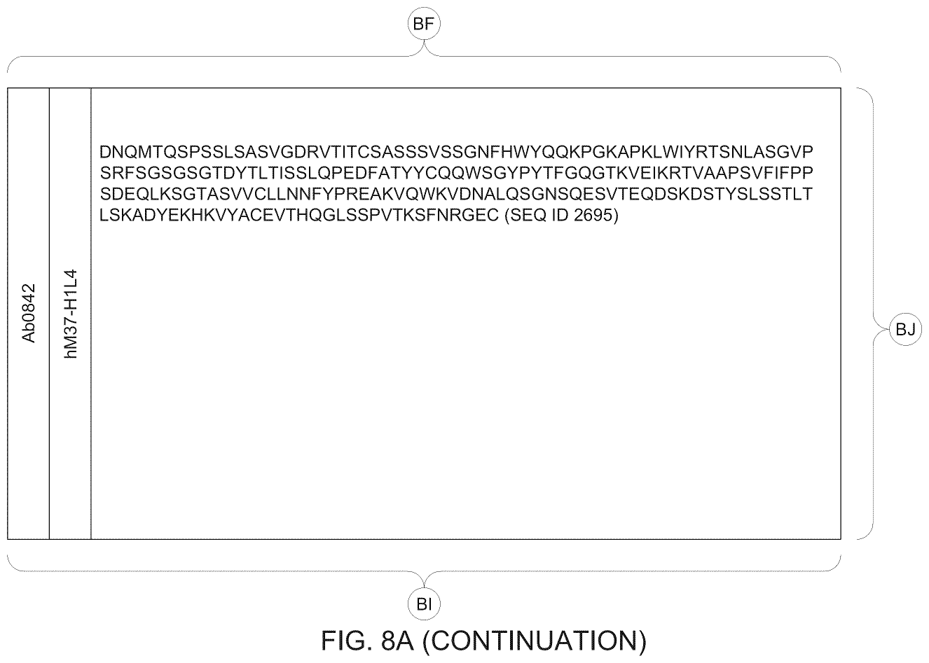

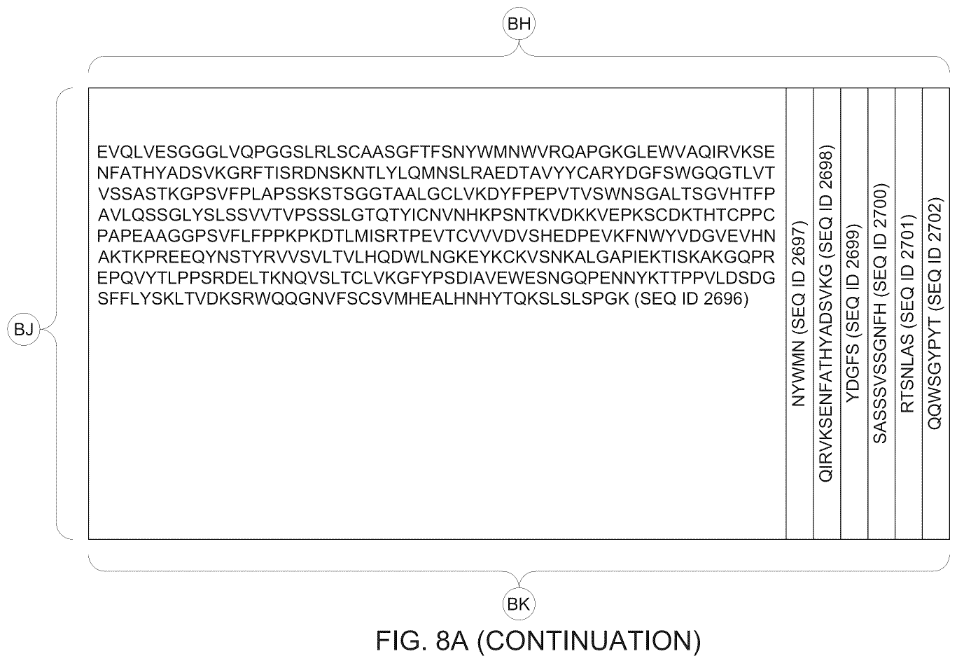

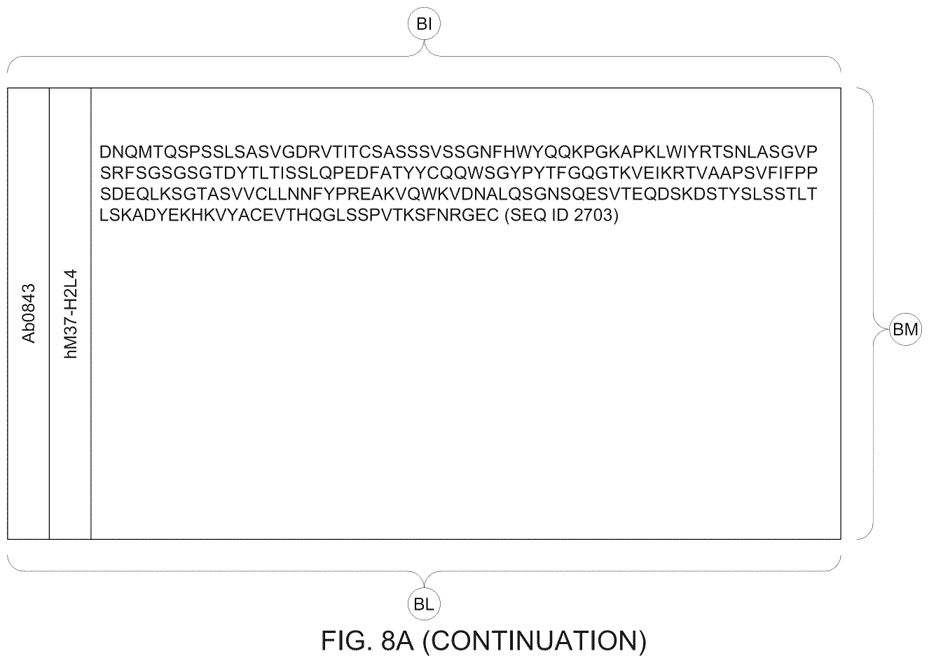

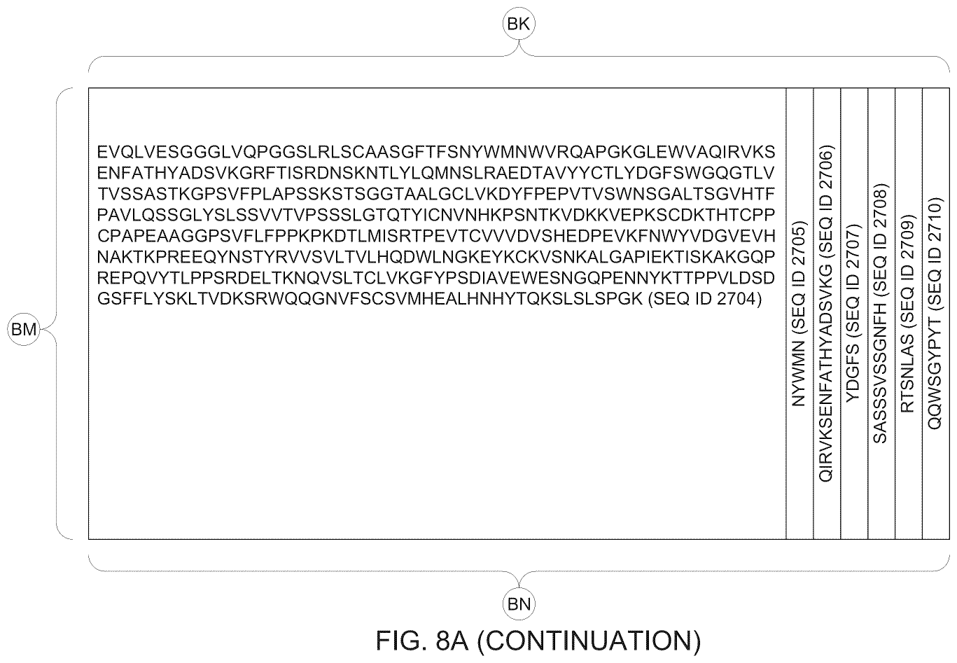

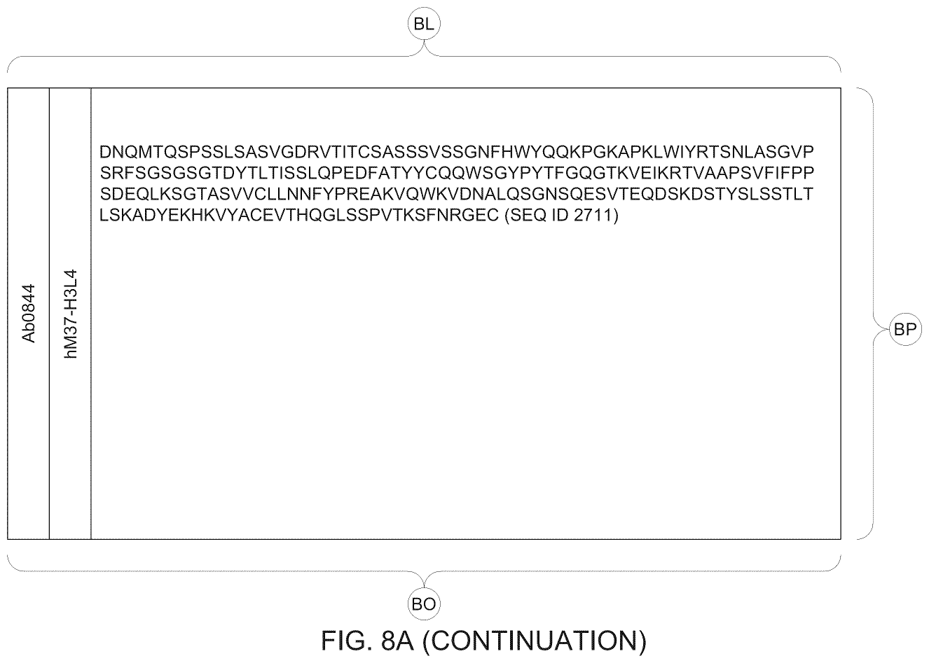

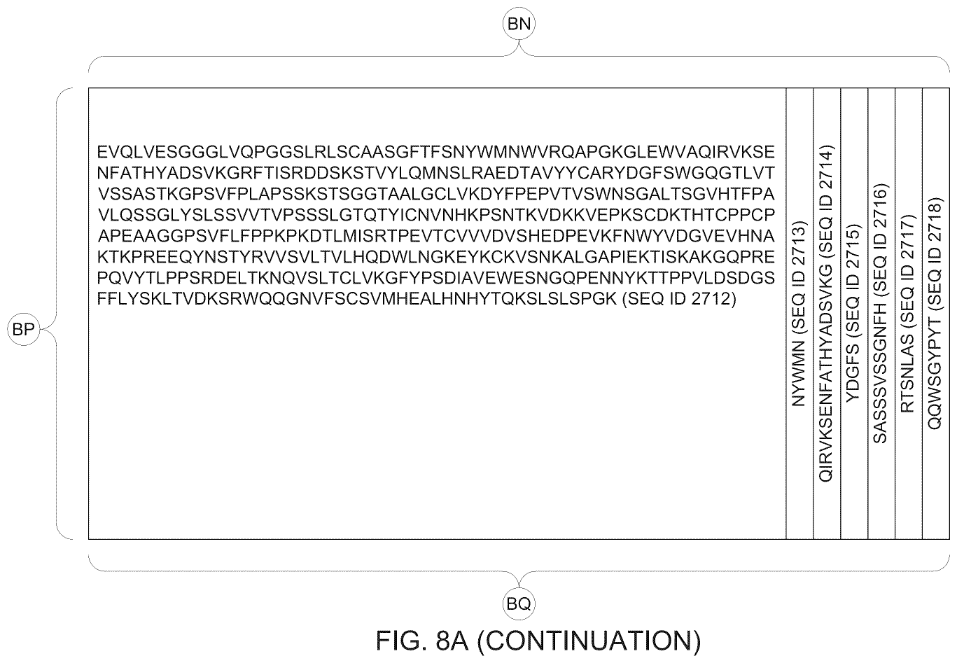

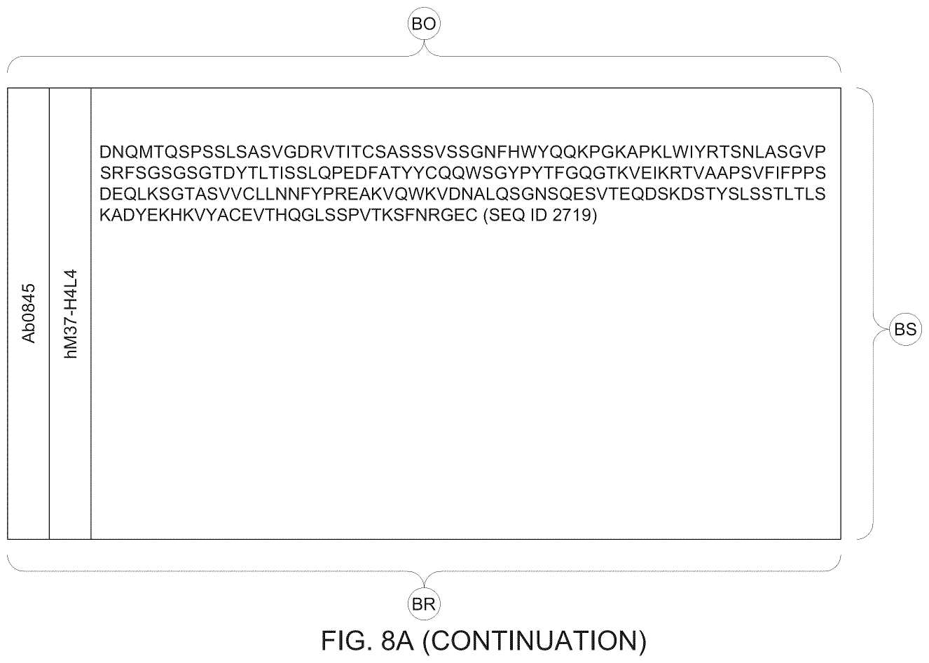

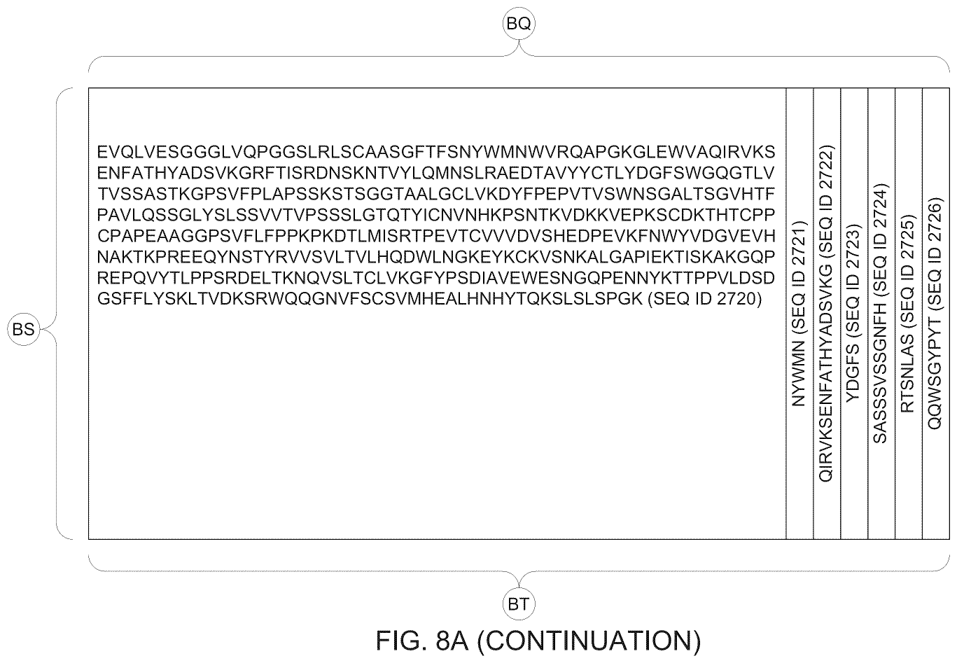

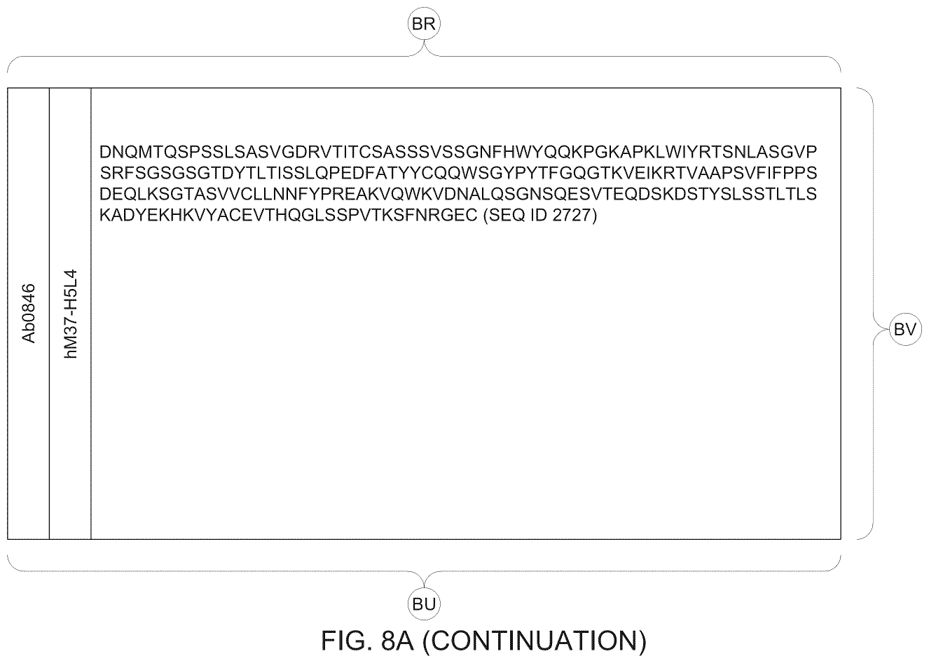

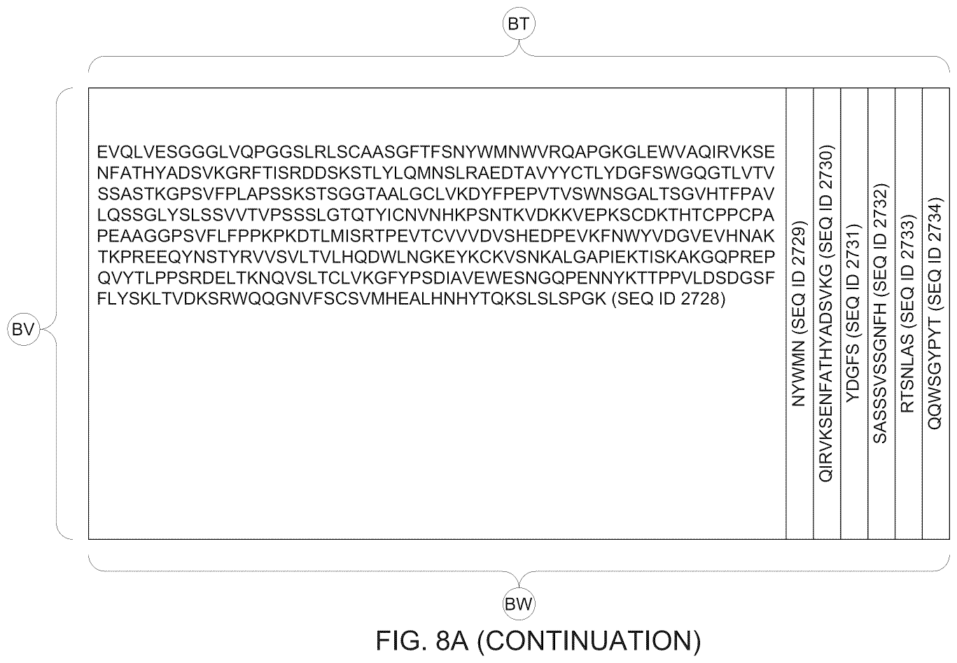

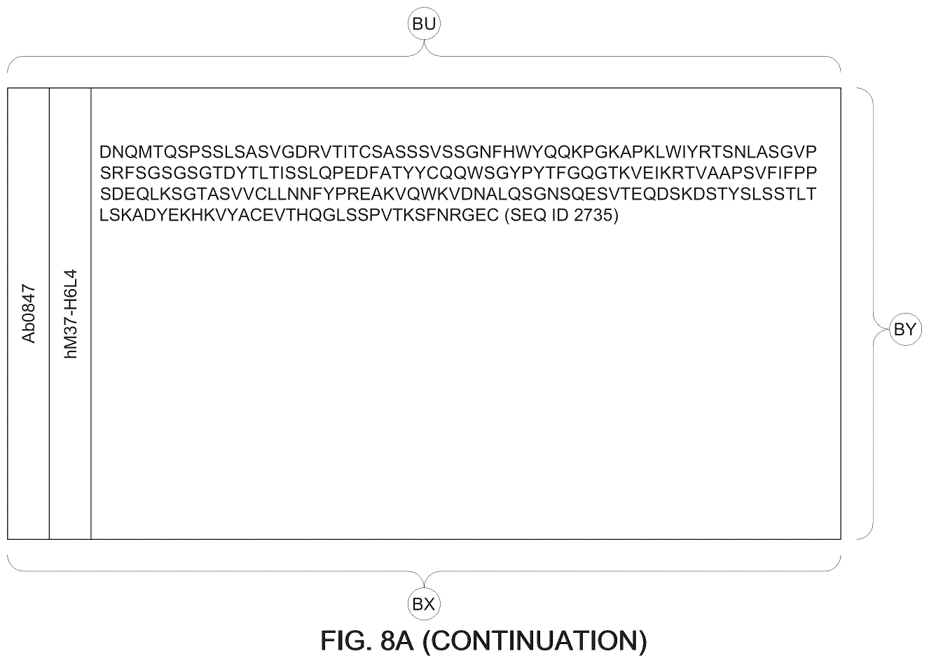

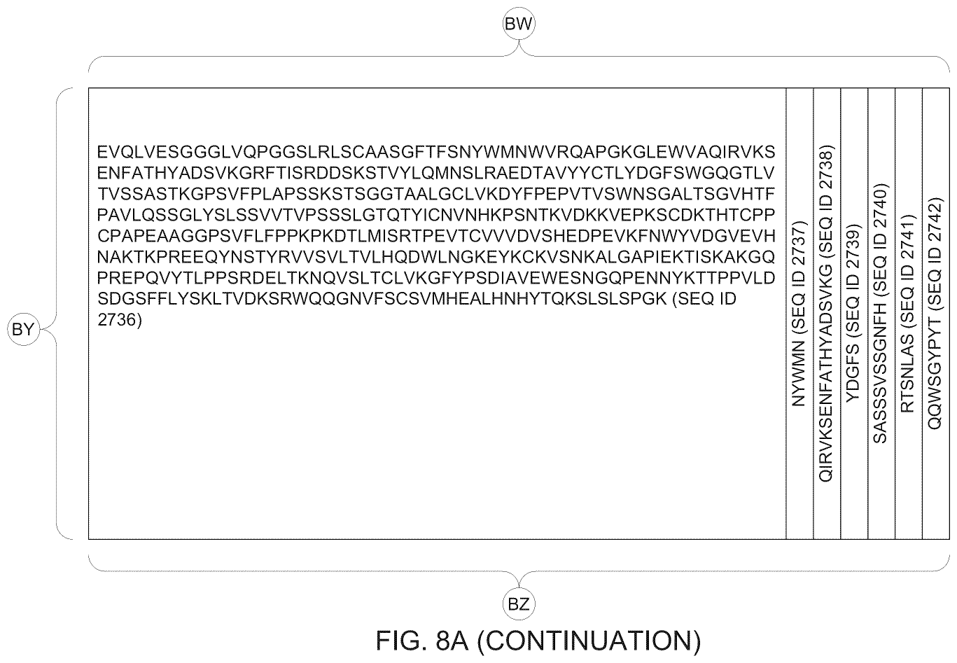

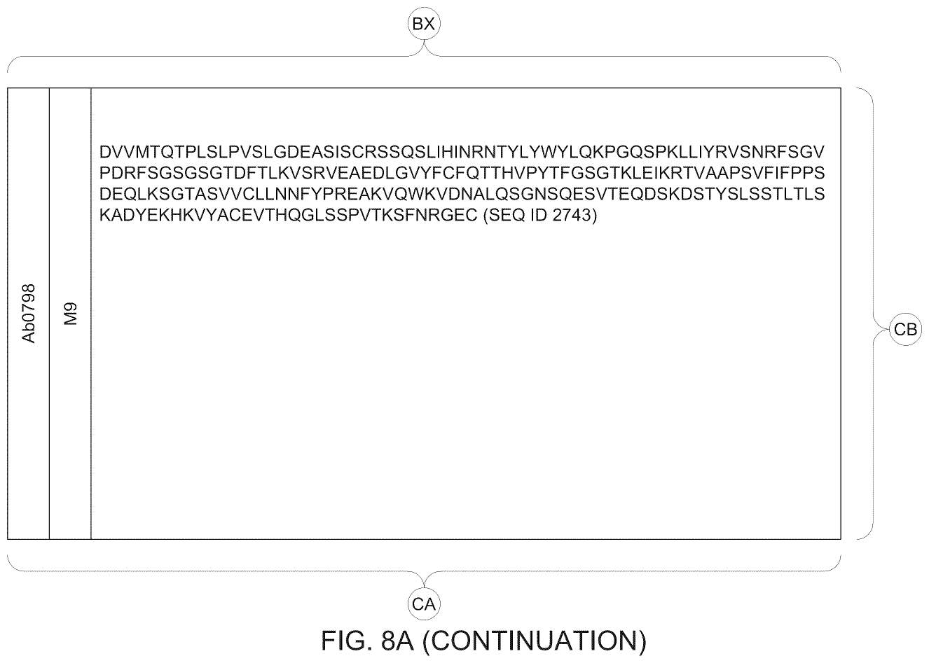

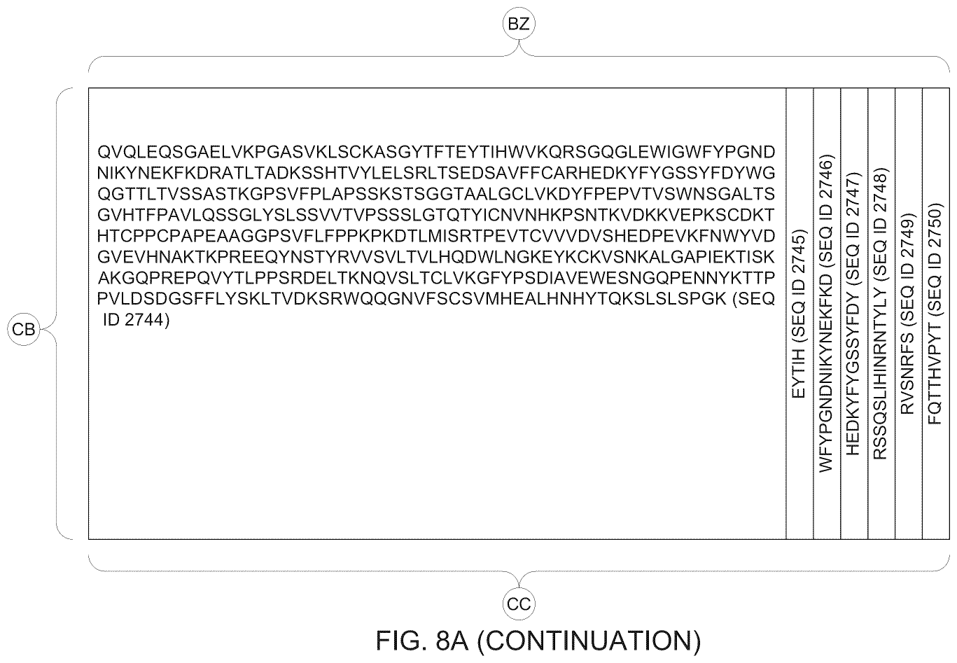

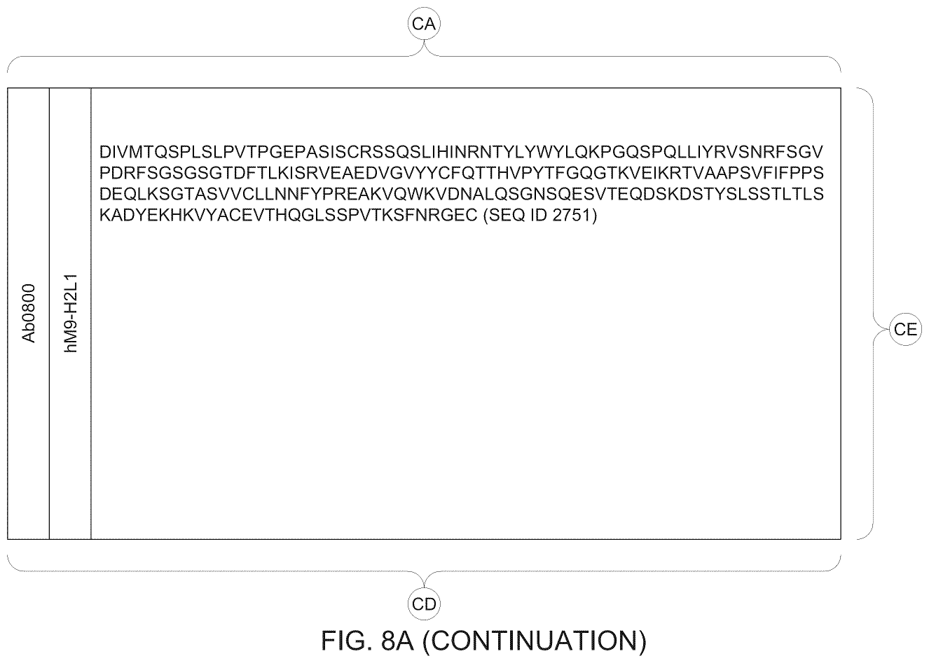

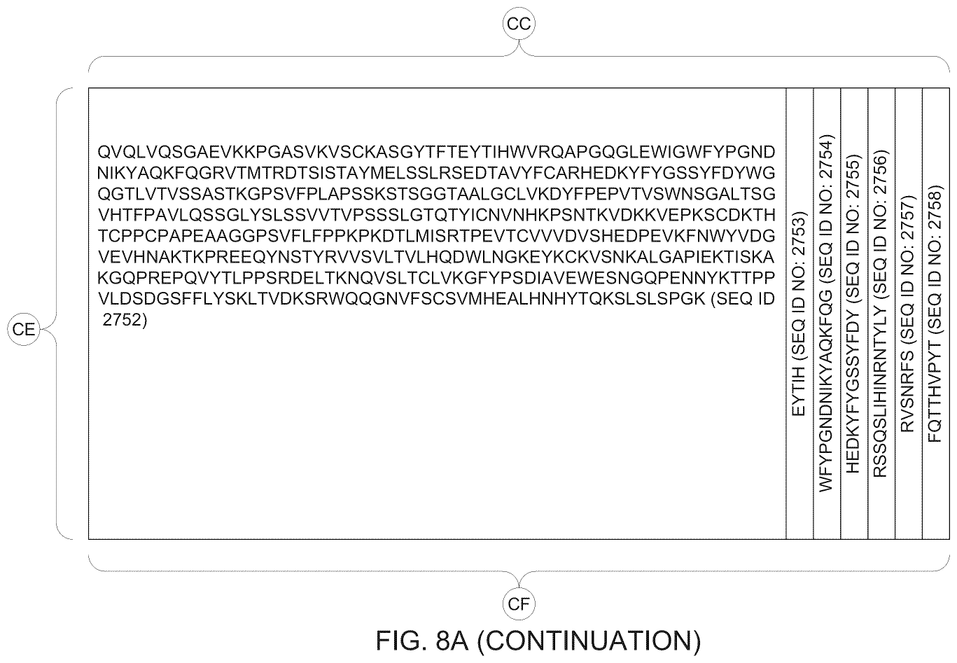

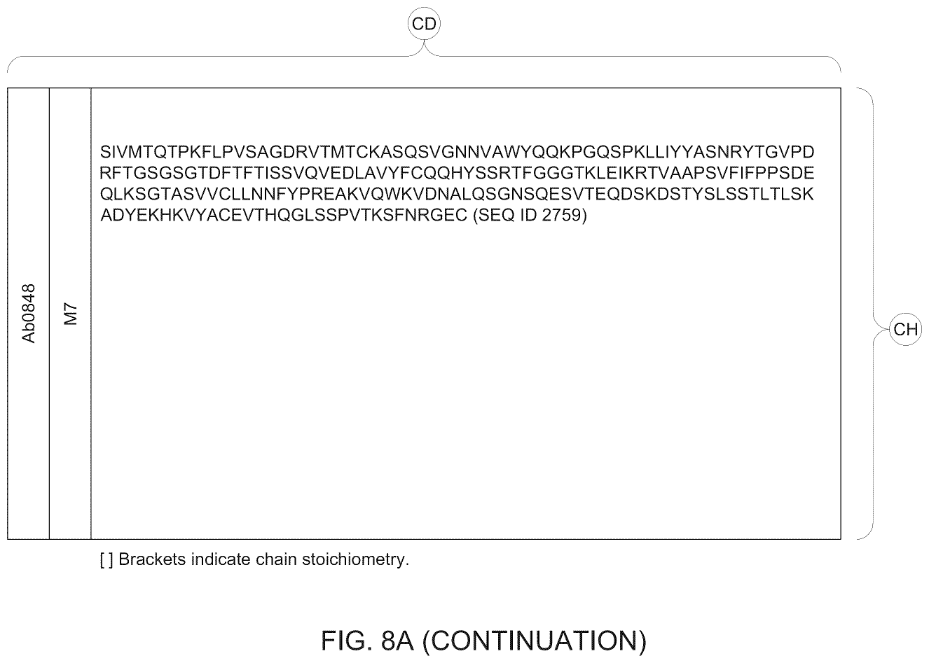

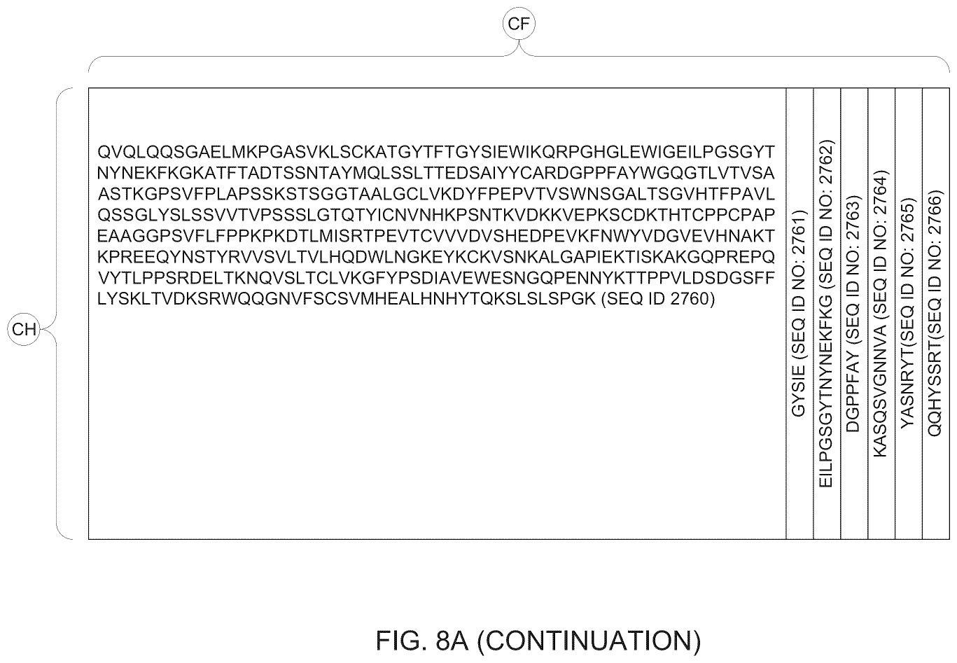

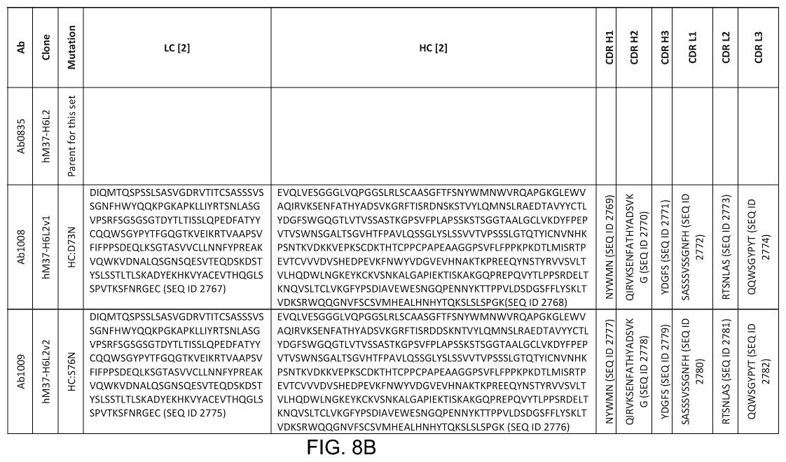

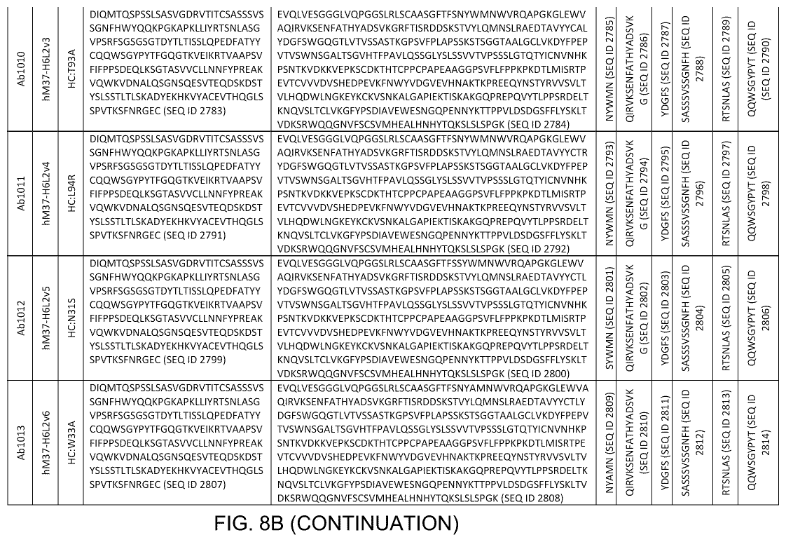

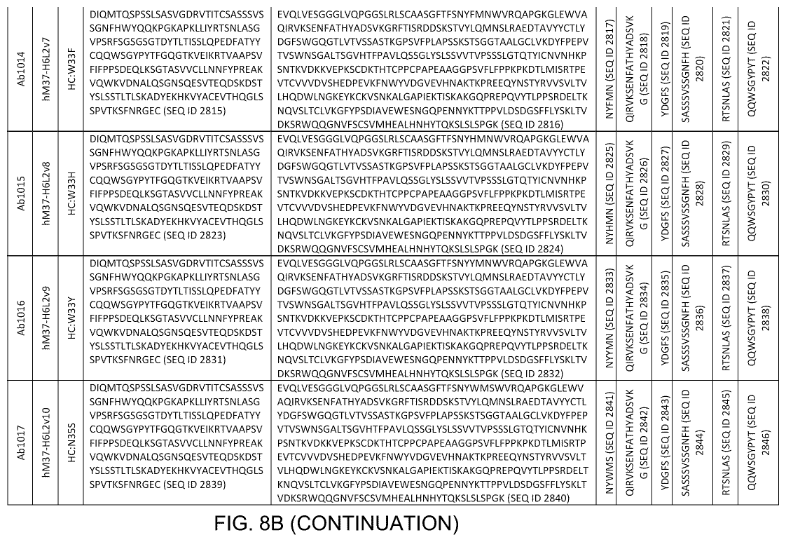

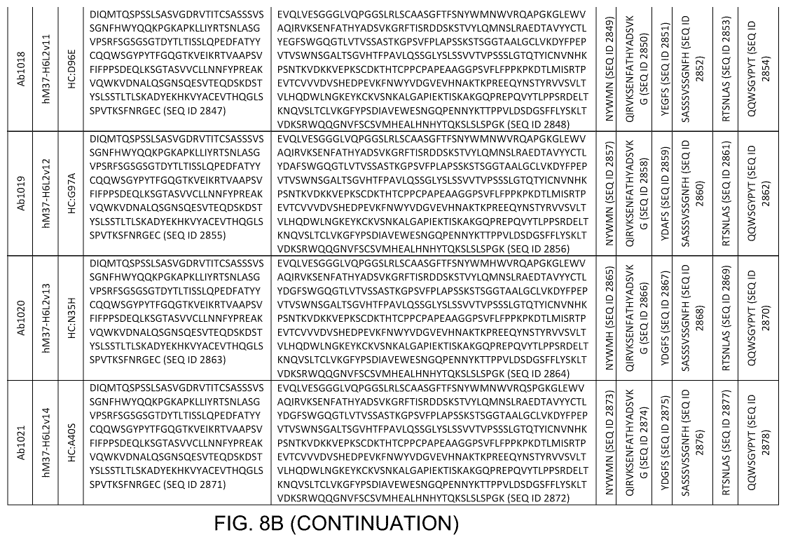

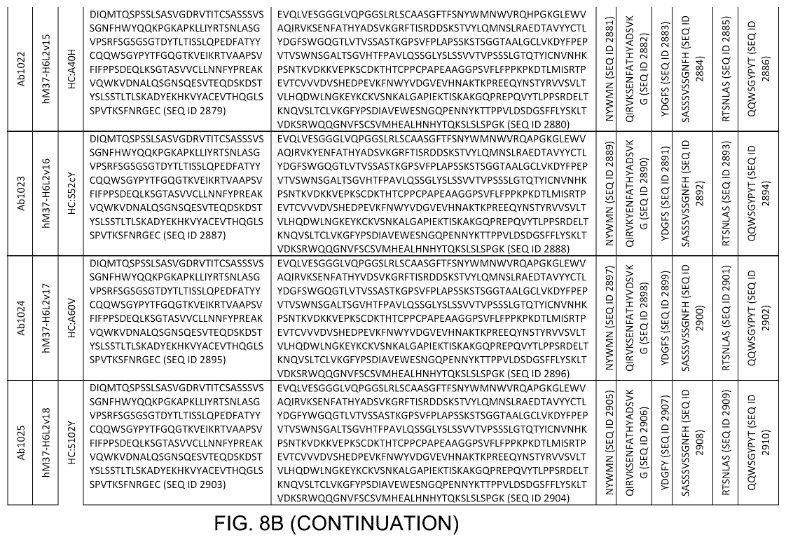

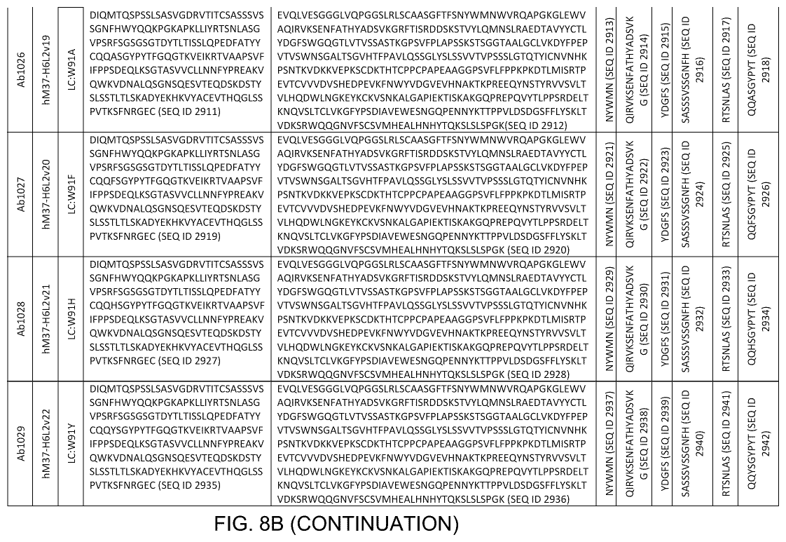

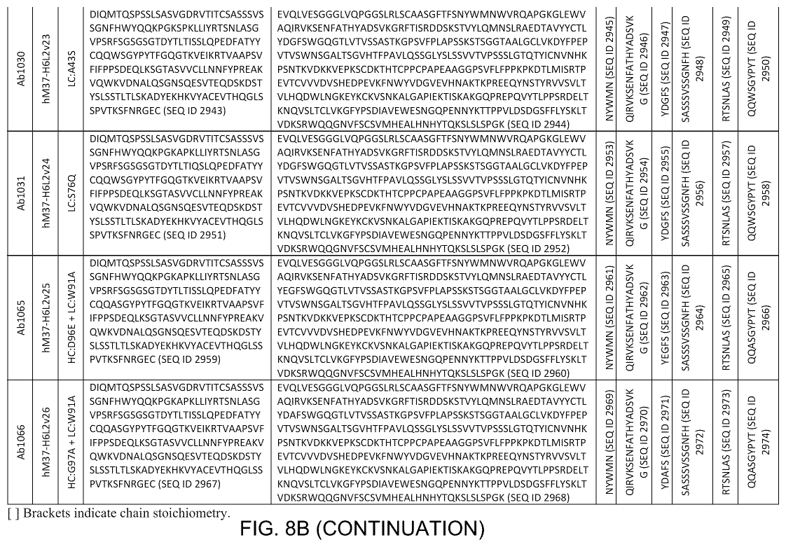

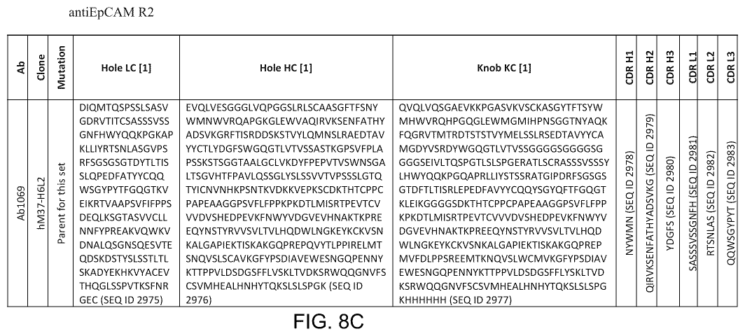

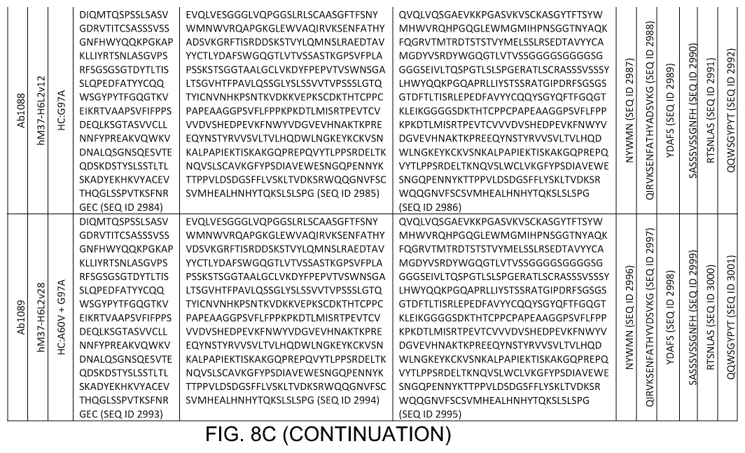

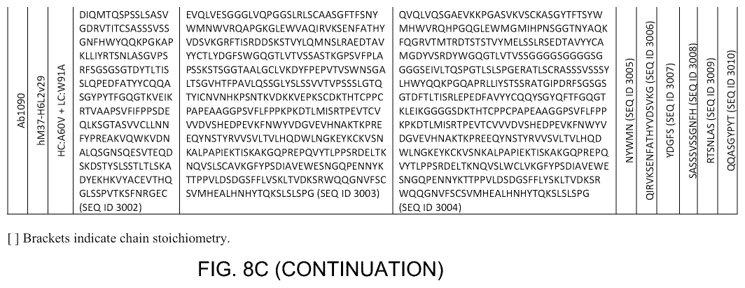

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 8A-8C optionally with one or more mutations. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 8A-8E optionally with one or more mutations at positions corresponding to one or more of positions 60 and 97 in the HC domain and 91 in the LC domain of Ab1090. In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from FIGS. 8A-8C.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from Ab0682, Ab0823, Ab0824, Ab0825, Ab0826, Ab0827, Ab0828, Ab0829, Ab0830, Ab0831, Ab0832, Ab0833, Ab0834, Ab0835, Ab0836, Ab0837, Ab0838, Ab0839, Ab0840, Ab0841, Ab0842, Ab0843, Ab0844, Ab0845, Ab0846, Ab0847, Ab1008, Ab1009, Ab1010, Ab1012, Ab1014, Ab1016, Ab1017, Ab1018, Ab1019, Ab1020, Ab1021, Ab1022, Ab1023, Ab1024, Ab1025, Ab1026, Ab1027, Ab1028, Ab1029, Ab1030, Ab1031, Ab1066, Ab1069, Ab1088, Ab1089, Ab1090 of FIGS. 8A-8C. In some embodiments, the sequences are from A0682. In some embodiments, the sequences are from Ab0823. In some embodiments, the sequences are from Ab0824. In some embodiments, the sequences are from Ab0825. In some embodiments, the sequences are from Ab0826. In some embodiments, the sequences are from Ab0827. In some embodiments, the sequences are from Ab0828. In some embodiments, the sequences are from Ab0829. In some embodiments, the sequences are from Ab0830. In some embodiments, the sequences are from Ab0831. In some embodiments, the sequences are from Ab0832. In some embodiments, the sequences are from Ab0833. In some embodiments, the sequences are from Ab0834. In some embodiments, the sequences are from Ab0835. In some embodiments, the sequences are from Ab0836. In some embodiments, the sequences are from Ab0837. In some embodiments, the sequences are from Ab0838. In some embodiments, the sequences are from Ab0839. In some embodiments, the sequences are from Ab0840. In some embodiments, the sequences are from Ab0841. In some embodiments, the sequences are from Ab0842. In some embodiments, the sequences are from Ab0843. In some embodiments, the sequences are from Ab0844. In some embodiments, the sequences are from Ab0845. In some embodiments, the sequences are from Ab0846. In some embodiments, the sequences are from Ab0847. In some embodiments, the sequences are from Ab1008. In some embodiments, the sequences are from Ab1009. In some embodiments, the sequences are from Ab1010. In some embodiments, the sequences are from Ab1012. In some embodiments, the sequences are from Ab1012. In some embodiments, the sequences are from Ab1014. In some embodiments, the sequences are from Ab1016. In some embodiments, the sequences are from Ab1017. In some embodiments, the sequences are from Ab1018. In some embodiments, the sequences are from Ab1019. In some embodiments, the sequences are from Ab1020. In some embodiments, the sequences are from Ab1021. In some embodiments, the sequences are from Ab1022. In some embodiments, the sequences are from Ab1023. In some embodiments, the sequences are from Ab1024. In some embodiments, the sequences are from Ab1025. In some embodiments, the sequences are from Ab1026. In some embodiments, the sequences are from Ab1027. In some embodiments, the sequences are from Ab1028. In some embodiments, the sequences are from Ab1029. In some embodiments, the sequences are from Ab1030. In some embodiments, the sequences are from Ab1031. In some embodiments, the sequences are from Ab1066. In some embodiments, the sequences are from Ab1069. In some embodiments, the sequences are from Ab1088. In some embodiments, the sequences are from Ab1089. In some embodiments, the sequences are from Ab1090.

In some embodiments, the vhCDR1, vhCDR2 and vhCDR3 sequences and said vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1090 optionally with one or more mutations at positions corresponding to one or more of positions 60 and 97 in the HC domain and 91 in the LC domain.

In some embodiments, one or more of the vhCDR1, vhCDR2 and vhCDR3 sequences and the vlCDR1, vlCDR2, and vlCDR3 sequences may be selected from the group consisting of the vhCDR1, vhCDR2 and vhCDR3 sequences and the vlCDR1, vlCDR2, and vlCDR3 sequences from the Ab1090. In some embodiments, the EpCAM binding domain may comprise a VH domain and VL domain selected from the group consisting of the VH and VL domains from FIGS. 8A-8C optionally with one or more mutations. In some embodiments, the EpCAM binding domain may comprise a VH domain and VL domain selected from the group consisting of the VH and VL domains from FIGS. 8A-8C.

In some embodiments, the EpCAM binding domain may comprise a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with the VH domain sequence of the Ab1090 and/or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity with the VL domain sequence of the Ab1090. In some embodiments, the EpCAM binding domain may comprise the VH and VL domain sequences of the Ab1090. In some embodiments, the EpCAM binding domain may comprise the Ab1090.

In some embodiments, the vhCDR1, vhCDR2 and vhCDR3 sequences and said vlCDR1, vlCDR2, and vlCDR3 sequences may comprise one or more mutations selected from the group consisting of mutations corresponding to HC:D73N, HC:S76N, HC:T93A, HC:N31S, HC:W33F, HC:W33H, HC:W33Y, HC:N35S, HC:D96E, HC:G97A, HC:N35H, HC:A40S, HC:A40H, HC:S52cY, HC:A60V, HC:S102Y, LC:W91A, LC:W91F, LC:W91H, LC:W91Y, LC:A43S, and LC:S76Q of the Ab0835. In some embodiments, the vhCDR1, vhCDR2 and vhCDR3 sequences and said vlCDR1, vlCDR2, and vlCDR3 sequences may comprise one or more mutations selected from the group consisting of mutations corresponding to G97A and A60V in the HC domain and W91A in the LC domain of the Ab1069. In some embodiments, the sequences have a mutation corresponding to said G97A. In some embodiments, the sequences have a mutation corresponding to said A60V. In some embodiments, the sequences have a mutation corresponding to said W91A.

In some embodiments, the EpCAM binding domain may comprise scFv means for binding indinavir. In some embodiments, the EpCAM binding domain may comprise Fab means for binding indinavir.

In some embodiments, said composition comprises an Fc domain with one or more knob variants. In some embodiments, said composition comprises an Fc domain with one or more hole variants. Some examples of the knob and hole Fc sequences are disclosed in FIG. 10.

In some embodiments, the Fc domain described herein having a knob variant comprises a sequence having at least 85, 86, 87, 88, 89, 90, 91, 92, 93, 94, 95, 96, 97, 98, 99 and 100% identity to any one of SEQ ID NO: 3235, 3237, 3239, 3241, and 3243. In some embodiments, the Fc domain described herein having a hole variant comprises a sequence having at least 85, 86, 87, 88, 89, 90, 91, 92, 93, 94, 95, 96, 97, 98, 99 and 100% identity to any one of SEQ ID NO: 3236, 3238, 3240, 3242, and 3244. In some embodiments, the Fc domain described herein having a knob variant comprises the sequence of 3235. In some embodiments, the Fc domain described herein having a knob variant comprises the sequence of 3237. In some embodiments, the Fc domain described herein having a knob variant comprises the sequence of 3239. In some embodiments, the Fc domain described herein having a knob variant comprises the sequence of 3241. In some embodiments, the Fc domain described herein having a knob variant comprises the sequence of 3243. In some embodiments, the Fc domain described herein having a hole variant comprises the sequence of SEQ ID NO: 3236. In some embodiments, the Fc domain described herein having a hole variant comprises the sequence of SEQ ID NO: 3238. In some embodiments, the Fc domain described herein having a hole variant comprises the sequence of SEQ ID NO: 3240. In some embodiments, the Fc domain described herein having a hole variant comprises the sequence of SEQ ID NO: 3242. In some embodiments, the Fc domain described herein having a hole variant comprises the sequence of SEQ ID NO: 3244.

In some embodiments, the composition may be a nucleic acid composition comprising a polynucleotide encoding a composition comprising an indinavir binding domain. In some embodiments, an expression vector composition comprising the nucleic acid composition. In some embodiments, a host cell may comprise the expression vector.

6. Co-Stimulatory Domains (CoS domain)

CTCoS binding proteins described in this invention comprise one or more T cell co-stimulatory domains (CoS domain). The CoS domains can be antigen binding domains (ABDs, generally the VH and VL domains that form an Fv) from antibodies or ligands that bind to and activate a costimulatory receptor on a T cell, and as result activate the T cell. Co-stimulatory receptors on T cells include, for example, CD28, ICOS, 4-1BB, OX40, CD27, CD40, CD40L, and GITR.

CTTCoS binding proteins described in this invention comprises one or more T cell co-stimulatory domains (CoS domain). The CoS domains can be antigen binding domains (ABDs, generally the VH and VL domains that form an Fv) from antibodies or ligands that bind to and activate a costimulatory receptor on a T cell, and as result activate the T cell. Co-stimulatory receptors on T cells include, for example, CD28, ICOS, 4-1BB, OX40, CD27, CD40, CD40L, and GITR.

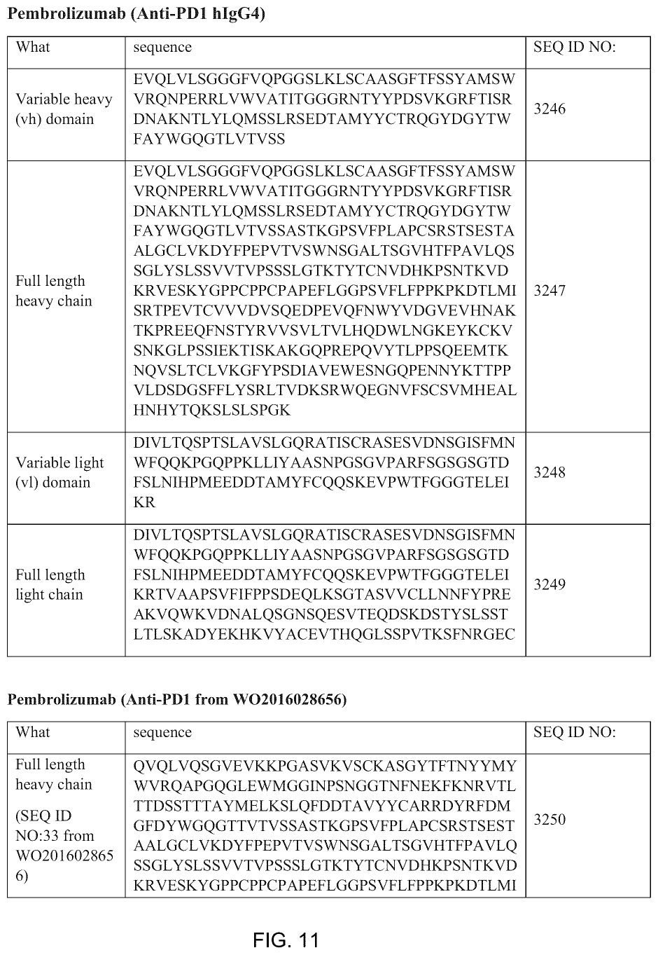

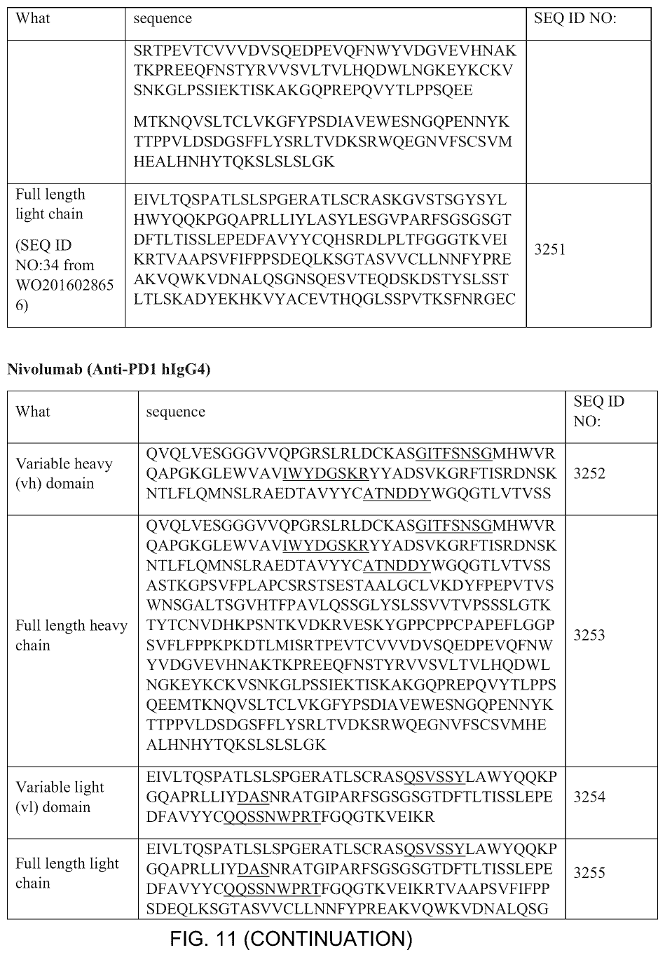

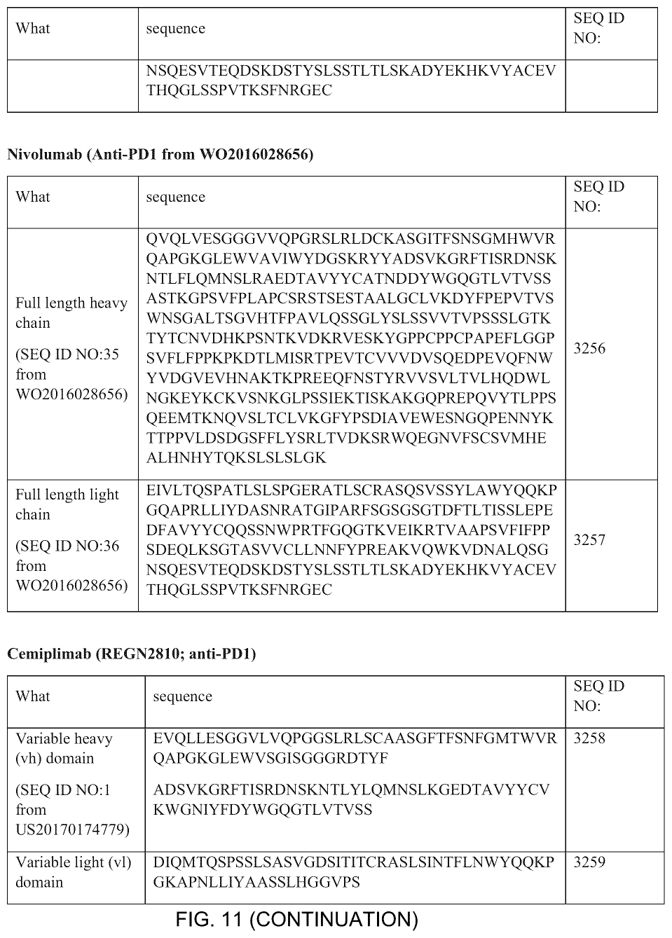

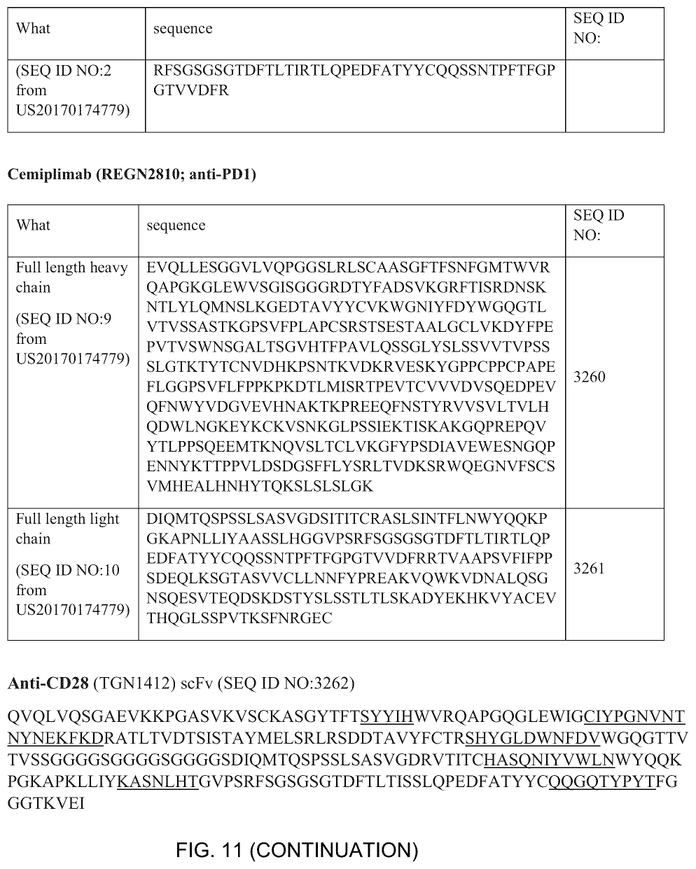

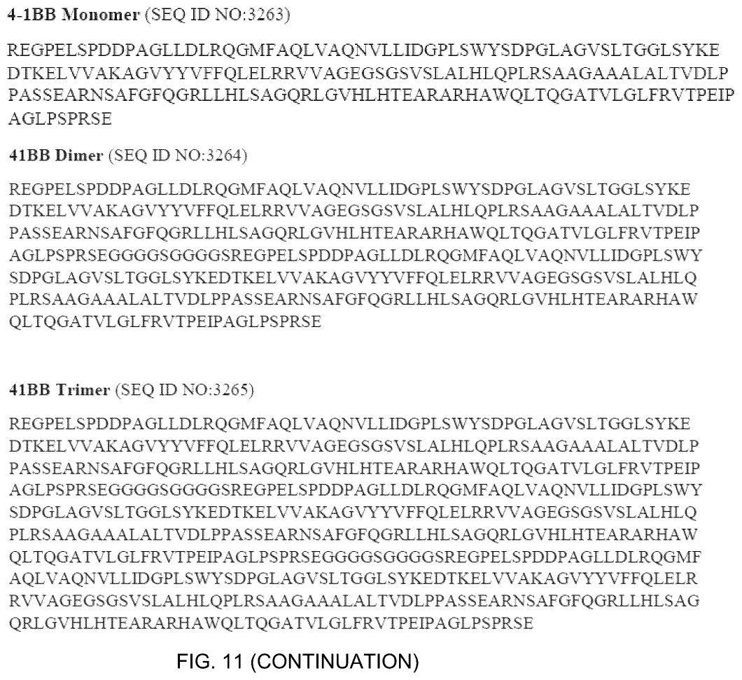

Accordingly, for example, the CoS domain can be an ABD comprising the variable heavy and variable light domains from an agonistic anti-CD28 antibody, including, for example, the sequence disclosed in FIG. 11.

In some embodiments, the CoS domain is a monomeric or trimeric 4-1BBL that binds 4-1BB. The amino acid sequence of the monomeric and trimeric 4-1BBL can be used. The CoS domain can also comprise an ABD comprising the variable heavy and variable light domains from an agonistic anti-4-1-BB antibody such as BMS-663513 urelumab. More anti-4-1BB antibodies can be found, for example, in U.S. Pat. No. 7,288,638 (incorporated by reference herein in its entirety and in particular for the anti-4-1BB variable heavy and variable light domain sequences disclosed therein).

The CoS domain can be ICOS-L (CD275) that binds ICOS. The CoS domain can also be an ABD from an anti-ICOS antibody that activates ICOS, such as the ABD comprising the variable heavy and variable light domains from MEDI-570 or JTX-2011.

In some embodiments, the CoS domain is OX40L (CD252) that binds OX40. The CoS can also include an ABD comprising the variable heavy and variable light domains from an anti-OX40 antibody that activates OX40 (see, for example, WO 2006/029879 or WO 2010/096418, incorporated by reference herein in their entireties and in particular for the anti-OX40 variable heavy and variable light domain sequences).

In some embodiments, the CoS domain includes an ABD from an anti-GITR antibody that activates GITR such as TRX518 (see, for example, U.S. Pat. No. 7,812,135, incorporated by reference herein in its entirety and in particular for the anti-OX40 variable heavy and variable light domain sequences).

In some embodiments, the CoS domain is CD70 that binds CD27. The CoS domain can also include an ABD from an anti-CD27 antibody that activates CD27, such as varlilumab CDX-1127 (see, for example, WO 2016/145085 and U.S. Patent Publication Nos. US 2011/0274685 and US 2012/0213771, incorporated by reference herein in their entireties and in particular for the anti-CD27 variable heavy and variable light domain sequences).

The CoS domain can be CD40L (CD154) that binds CD40. The CoS domain can be CD40 that binds CD40L. The CoS domain can include an ABD from an agonistic antibody targeting CD40, such as CP-870,893, lucatumumab, dacetuzumab.

The CoS domains can include antigen binding domains or ligands that bind to and inhibit a coinhibitory receptor on a T cell, and as result activating the T cell. Co-inhibitory receptors on T cells include, for example, PD-1, CTLA4, LAG3, B7-H1, B7-1, CD160, BTLA, LAIR1, TIM3, 2B4, and TIGIT.

For example, the CoS domain used herein can be an ABD from an inhibitory antibody that binds to PD-1, including, but not limited to, nivolumab, BMS-936558, MDX-1106, ONO-4538, AMP224, CT-011, and MK-3475 (pembrolizumab), cemiplimab (REGN2810), SHR-1210 (CTR20160175 and CTR20170090), SHR-1210 (CTR20170299 and CTR20170322), JS-001 (CTR20160274), IBI308 (CTR20160735), BGB-A317 (CTR20160872) and/or a PD-1 antibody as recited in U.S. Patent Publication No. 2017/0081409. There are two approved anti-PD-1 antibodies, pembrolizumab (Keytruda®; MK-3475-033) and nivolumab (Opdivo®; CheckMate078) and many more in development, the ABDs of which can be used in this invention. Exemplary anti-PD-1 antibody sequences are shown in FIG. 11.

In some embodiments, the CoS domain comprises an ABD from an anti-CTLA4 antibody, such as ipilimumab, tremelimumab. In some embodiments, the CoS domain comprises the ABD from an anti-LAG-3 antibody such as IMP-321.

In some embodiments, the CoS domain comprises an ABD from an anti-TIM-3 antibody (see, for example, WO 2013/006490 or U.S. Patent Publication No US 2016/0257758, incorporated by reference herein in their entireties, and in in particular for the anti-TIM-3 variable heavy and variable light domain sequences).

### 7. Format 1 Complexes

As generally outlined herein, the Format 1 inventions provide pairs of binding proteins (e.g. a CC binding protein and a CT binding protein) that together, in the presence of a iCID-SM, form a T cell engaging complex. Generally, each binding protein is in turn made up of either two fusion polypeptides (that together form either a CC binding protein or a CT binding protein), or a monomeric fusion polypeptide as outlined below. As will be appreciated by those in the art, the CC binding proteins and the CT binding proteins can each be independently selected from monomeric fusion polypeptides, homodimeric fusion polypeptides and heterodimeric fusion polypeptides.

a. CC Binding Proteins

Accordingly, the present invention provides CC fusion polypeptides that form the CC binding protein(s) of the invention. Each CC binding protein contains one or more first iCID domain, and an anti-CD3 ABD. In some embodiments, the CC binding protein does not contain an Fc domain, such as direct fusion of a first iCID domains and an αCD3-ABD. Both the iCID domain and the αCD3 ABD can take the format of an scFv, a Fab, an scFab or a single domain antibody such as the VHH of camelid derived single domain antibody. In some embodiments, the CC binding protein contains an Fc domain. In some cases, the CC binding protein is monomeric. It can include an iCID domain directly fused to an αCD3-ABD, or it can also rely on the use of a monomeric Fc domain, as more fully outlined below. In some embodiments, the CC binding protein comprises a first and a second CC fusion polypeptide, that come together as dimers, either heterodimeric or homodimeric, to provide the αCD3-ABD functionally coupled to an iCID domain.

### (i) Monomeric CC Fusion Polypeptides

In some embodiments, the CC binding protein is monomeric and relies on the use of a monomeric IgG4 Fc domain. In some embodiments, the CC binding proteins are monomeric proteins comprising one or more iCID domain, an αCD3-ABD, optional domain linker(s) and an IgG4 monomeric Fc domain. The CC binding polypeptide can be a fusion polypeptide with a structure selected from the group, from N- to C-terminal: CID domain-optional domain linker-αCD3 ABD-optional domain linker-Fc domain; αCD3 ABD-optional domain linker-iCID domain-optional domain linker-Fc domain; iCID domain-optional domain linker-Fc domain-optional domain linker-αCD3-ABD; αCD3 ABD-optional domain linker-Fc domain-optional domain linker-iCID domain; Fc domain-optional domain linker-αCD3-ABD-optional domain linker-iCID domain; and Fc domain-optional domain linker-iCID domain-optional domain linker-αCD3 ABD; iCID domain-optional domain linker-iCID domain-optional domain linker-αCD3 ABD-optional domain linker-Fc domain; and iCID domain-optional domain linker-iCID domain-optional domain linker-αCD3 ABD-optional domain linker-Fc domain. Either or both of the iCID and αCD3 ABD can take any one of the formats including Fab, scFv, scFab, a single domain antibody such as the VHH of camelid derived single domain antibody.

In some instances, the selected arrangements of domains of the monomeric CC binding protein employed in the T-LITE composition provide an improvement, e.g., in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

### (ii) Dimeric CC Binding Proteins

In some embodiments, the CC binding protein comprises a first and a second CC fusion polypeptide, that come together as dimers, either heterodimeric or homodimeric, to provide the αCD3-ABD functionally coupled to a iCID domain. In these embodiments, the CC binding proteins rely on the use of Fc domains that are dimers, either heterodimeric Fc domains or homodimeric Fc domains.

In some embodiments, the CC binding proteins are CC heterodimeric binding proteins that use heterodimerization variants in the Fc domains. Thus, in some embodiments, the CC binding protein comprises a first and a second CC fusion polypeptide, wherein one of the first and second CC fusion polypeptides contains the αCD3-ABD and the other the iCID domain. In some embodiments, the CC binding protein comprises a first CC fusion polypeptide which contains both the αCD3-ABD and the iCID domain, and a second CC fusion polypeptide comprising an empty Fc domain. In these embodiments, the first and second CC fusion polypeptides can have the structures (from N- to C-terminal, with “DL” standing for “domain linker”) shown in Table 4. In some embodiments, each of the CC fusion polypeptide having one iCID domains may further comprise another iCID domain linked to the iCID domain via an optional DL, for example, as shown in 37-40 in Table 4 below. The iCID domains in the same fusion polypeptide may be identical. In some embodiments, the iCID domains in the same fusion polypeptide may be different. The DLs in the same fusion polypeptide may be different. In some embodiments, the DLs in the same fusion polypeptide may be same.

TABLE 4

| | First CC fusion polypeptide | Second CC fusion polypeptide |
|---|---|---|
| | (N- to C- terminal) | (N- to C- terminal) |
| 1 | Fc domain | iCID-DL-Fc domain |
| 2 | Fc domain | αCD3-ABD-DL-Fc domain |
| 3 | Fc domain | αCD3-ABD-DL-iCID-DL-Fc domain |
| 4 | Fc domain | iCID-DL-αCD3-ABD-DL-Fc domain |
| 5 | Fc domain | Fc domain-DL-iCID |
| 6 | Fc domain | Fc domain-DL-αCD3-ABD |
| 7 | Fc domain | Fc domain-DL-αCD3-ABD-DL-iCID |
| 8 | Fc domain | Fc domain-DL-iCID-DL-αCD3-ABD |
| 9 | iCID-DL-Fc domain | αCD3-ABD-DL-Fc domain |
| 10 | iCID-DL-Fc domain | αCD3-ABD-DL-iCID-DL-Fc domain |
| 11 | iCID-DL-Fc domain | iCID-DL-αCD3-ABD-DL-Fc domain |
| 12 | iCID-DL-Fc domain | Fc domain-DL-iCID |
| 13 | iCID-DL-Fc domain | Fc domain-DL-αCD3-ABD |
| 14 | iCID-DL-Fc domain | Fc domain-DL-αCD3-ABD-DL-iCID |
| 15 | iCID-DL-Fc domain | Fc domain-DL-iCID-DL-αCD3 ABD |
| 16 | αCD3-ABD-DL-Fc domain | αCD3 ABD-DL-iCID-DL-Fc domain |
| 17 | αCD3 ABD-DL-Fc domain | iCID-DL-αCD3 ABD-DL-Fc domain |
| 18 | αCD3 ABD-DL-Fc domain | Fc domain-DL-iCID |
| 19 | αCD3 ABD-DL-Fc domain | Fc domain-DL-αCD3 ABD |
| 20 | αCD3 ABD-DL-Fc domain | Fc domain-DL-αCD3 ABD-DL-iCID |
| 21 | αCD3 ABD-DL-Fc domain | Fc domain-DL-iCID-DL-αCD3 ABD |
| 22 | αCD3 ABD-DL-iCID-DL-Fc domain | iCID-DL-αCD3 ABD-DL-Fc domain |
| 23 | αCD3 ABD-DL-iCID-DL-Fc domain | Fc domain-DL-iCID |
| 24 | αCD3 ABD-DL-iCID-DL-Fc domain | Fc domain-DL-αCD3 ABD |
| 25 | αCD3 ABD-DL-iCID-DL-Fc domain | Fc domain-DL-αCD3 ABD-DL-iCID |
| 26 | αCD3 ABD-DL-iCID-DL-Fc domain | Fc domain-DL-iCID-DL-αCD3 ABD |
| 27 | iCID-DL-αCD3 ABD-DL-Fc domain | Fc domain-DL-iCID |
| 28 | iCID-DL-αCD3 ABD-DL-Fc domain | Fc domain-DL-αCD3 ABD |
| 29 | iCID-DL-αCD3 ABD-DL-Fc domain | Fc domain-DL-αCD3 ABD-DL-iCID |
| 30 | iCID-DL-αCD3 ABD-DL-Fc domain | Fc domain-DL-iCID-DL-αCD3 ABD |
| 31 | Fc domain-DL-iCID | Fc domain-DL-αCD3 ABD |
| 32 | Fc domain-DL-iCID | Fc domain-DL-αCD3 ABD-DL-iCID |
| 33 | Fc domain-DL-iCID | Fc domain-DL-iCID-DL-αCD3 ABD |
| 34 | Fc domain-DL-αCD3 ABD | Fc domain-DL-αCD3 ABD-DL-iCID |
| 35 | Fc domain-DL-αCD3 ABD | Fc domain-DL-iCID-DL-αCD3 ABD |
| 36 | Fc domain-DL-αCD3 ABD-DL-iCID | Fc domain-DL-iCID-DL-αCD3 ABD |
| 37 | iCID-DL-iCID-DL-Fc domain | αCD3-ABD-DL-Fc domain |
| 38 | iCID-DL-iCID-DL-Fc domain | Fc domain-DL-αCD3-ABD |
| 39 | Fc domain-DL-iCID-DL-iCID | αCD3-ABD-DL-Fc domain |
| 40 | Fc domain-DL-iCID-DL-iCID | Fc domain-DL-αCD3-ABD |

In some embodiments, the first CC fusion polypeptide may be iCID-DL-iCID-DL-Fc domain. In some embodiments, the first CC fusion polypeptide may be αCD3 ABD-DL-iCID-DL-iCID-DL-Fc domain. In some embodiments, the first CC fusion polypeptide may be iCID-DL-iCID-DL-αCD3 ABD-DL-Fc domain. In some embodiments, the first CC fusion polypeptide may be Fc domain-DL-iCID-DL-iCID. In some embodiments, the first CC fusion polypeptide may be Fc domain-DL-αCD3 ABD-DL-iCID-DL-iCID.

In some embodiments, the second CC fusion polypeptide may be iCID-DL-iCID-DL-Fc domain. In some embodiments, the second CC fusion polypeptide may be αCD3-ABD-DL-iCID-DL-iCID-DL-Fc domain. In some embodiments, the second CC fusion polypeptide may be iCID-DL-iCID-DL-αCD3-ABD-DL-Fc domain. In some embodiments, the second CC fusion polypeptide may be Fc domain-DL-iCID-DL-iCID. In some embodiments, the second CC fusion polypeptide may be Fc domain-DL-αCD3-ABD-DL-iCID-DL-iCID. In some embodiments, the second CC fusion polypeptide may be Fc domain-DL-iCID-DL-iCID-DL-αCD3-ABD.

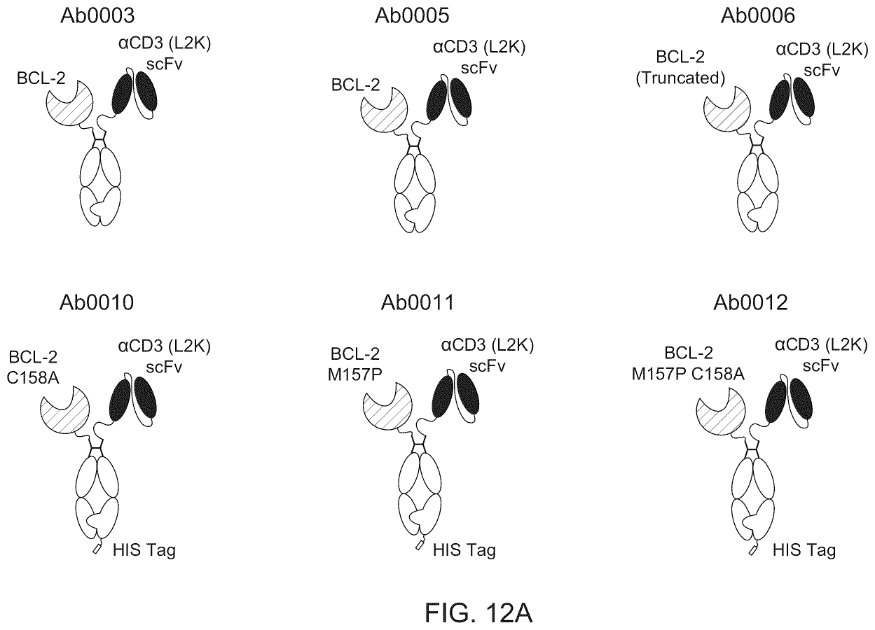

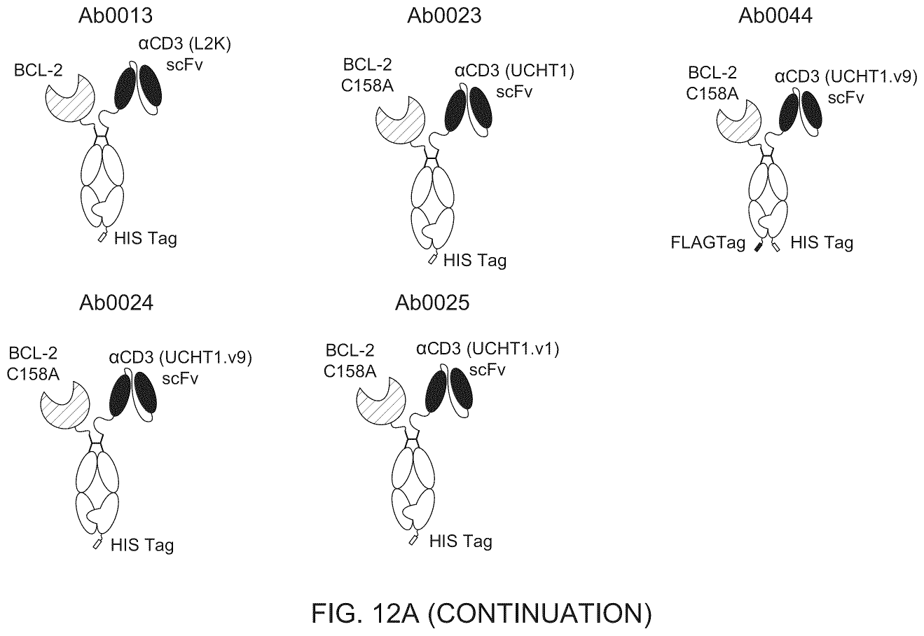

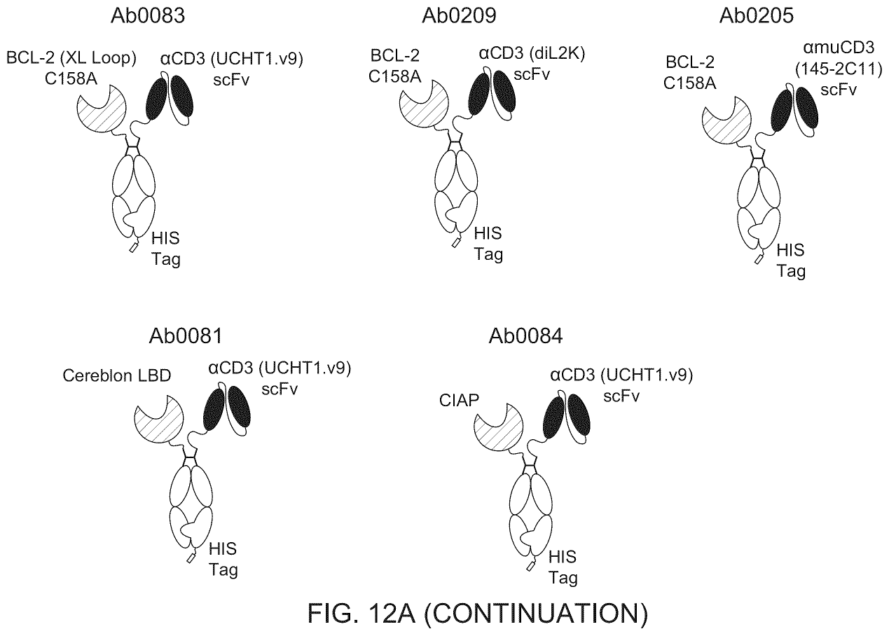

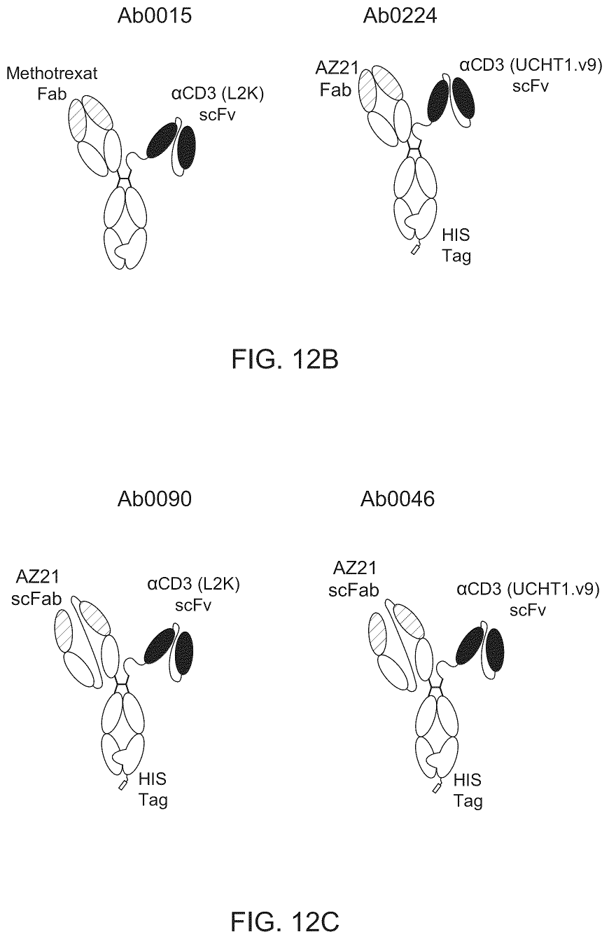

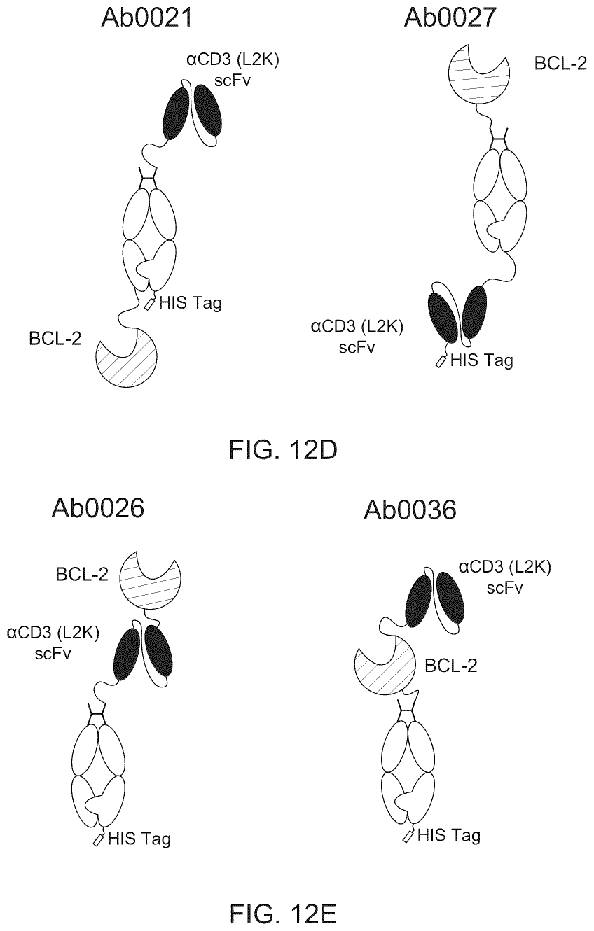

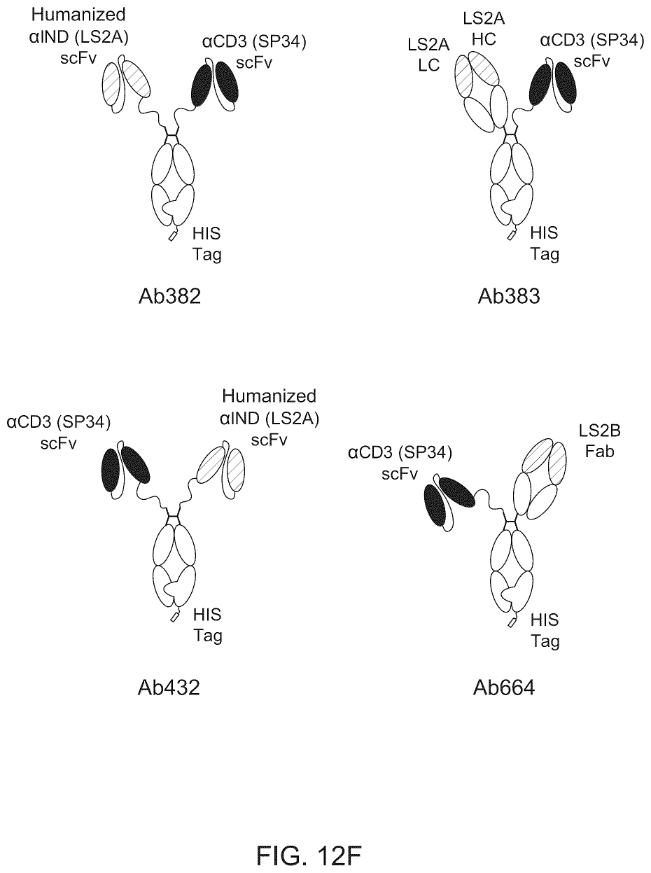

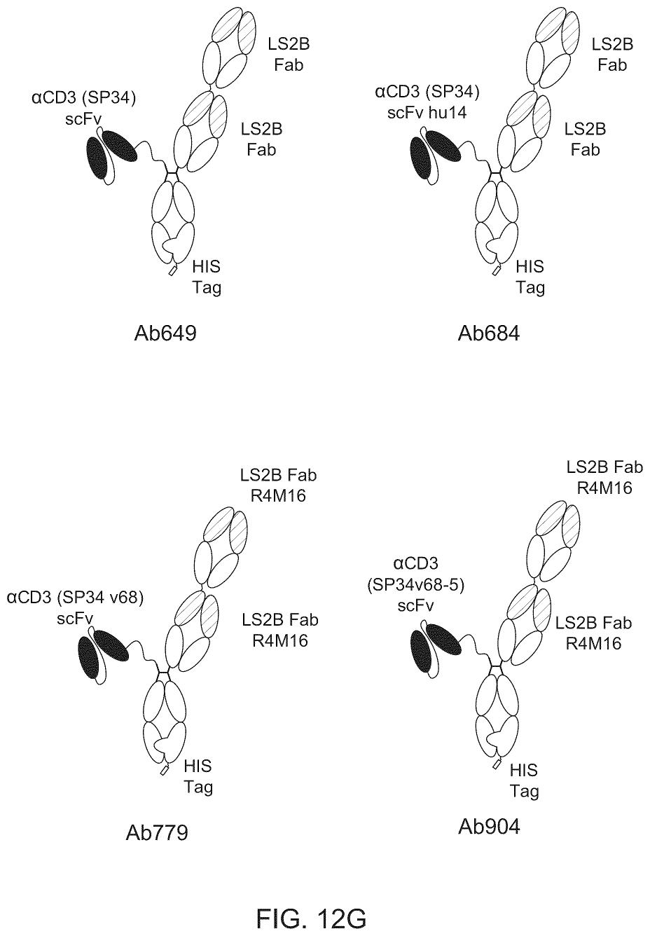

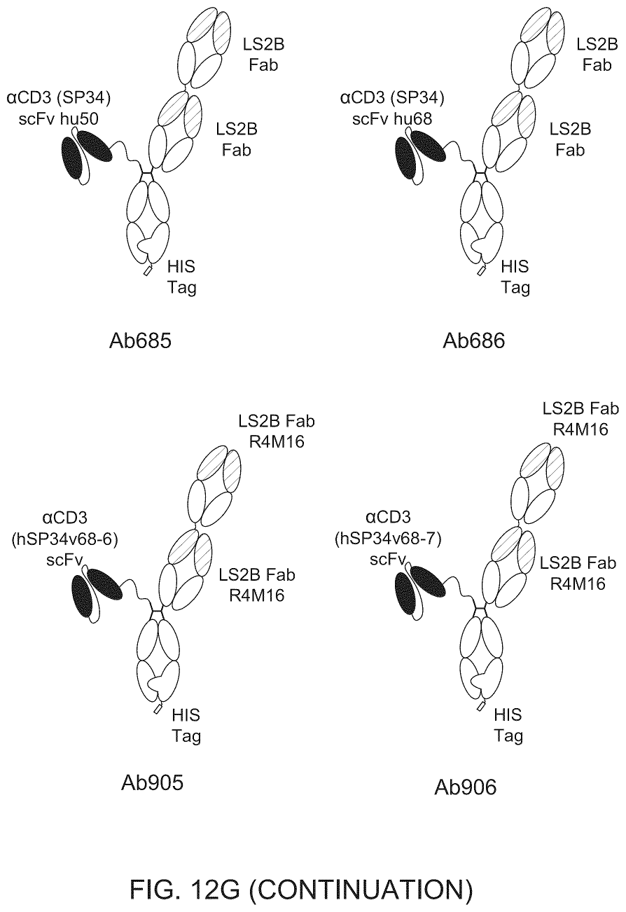

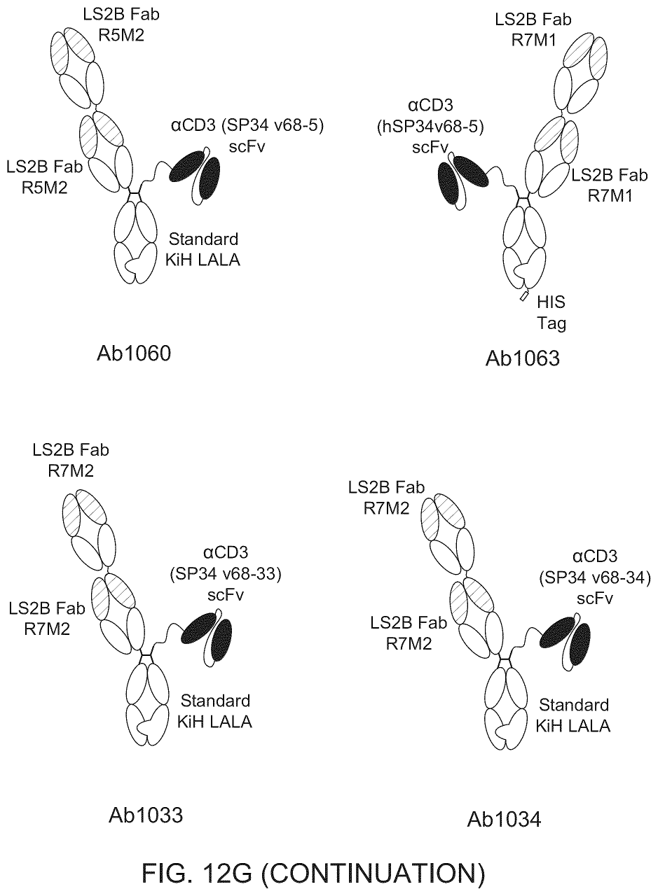

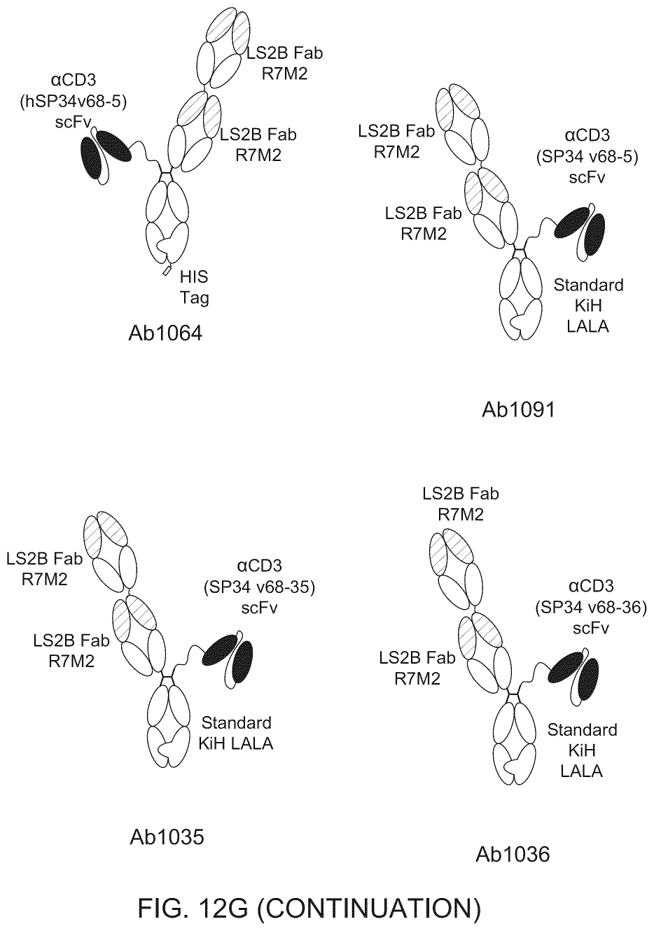

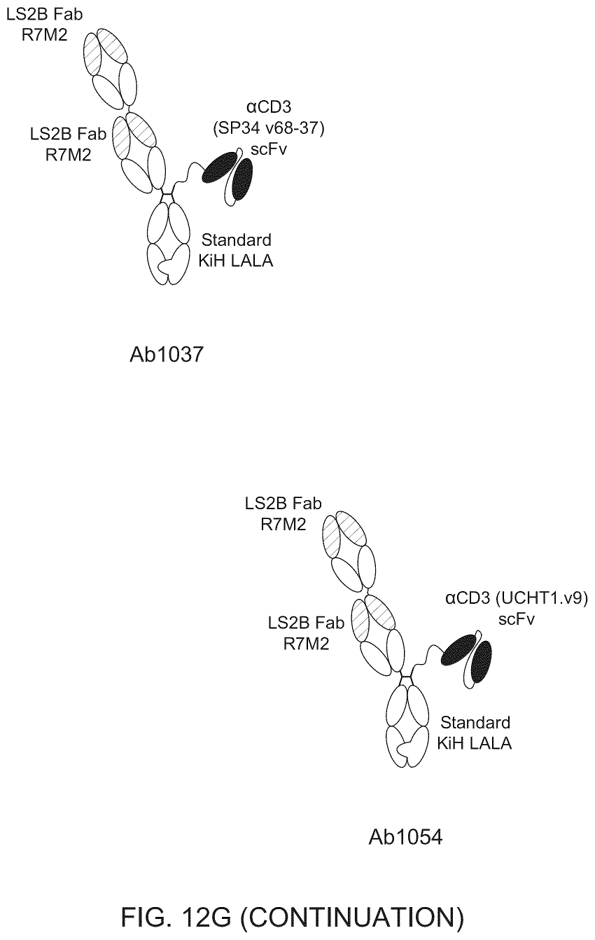

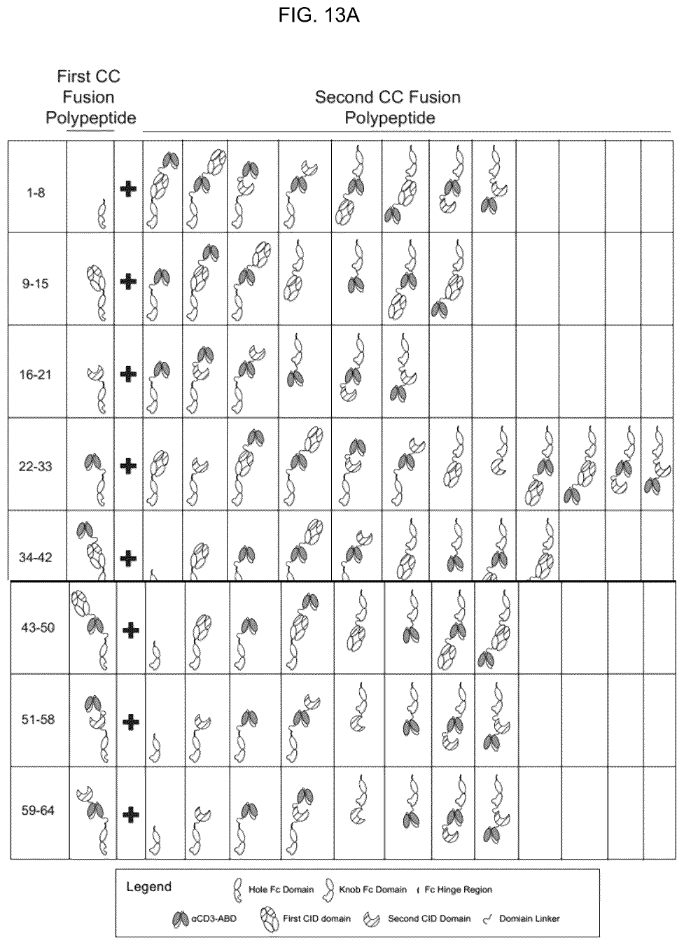

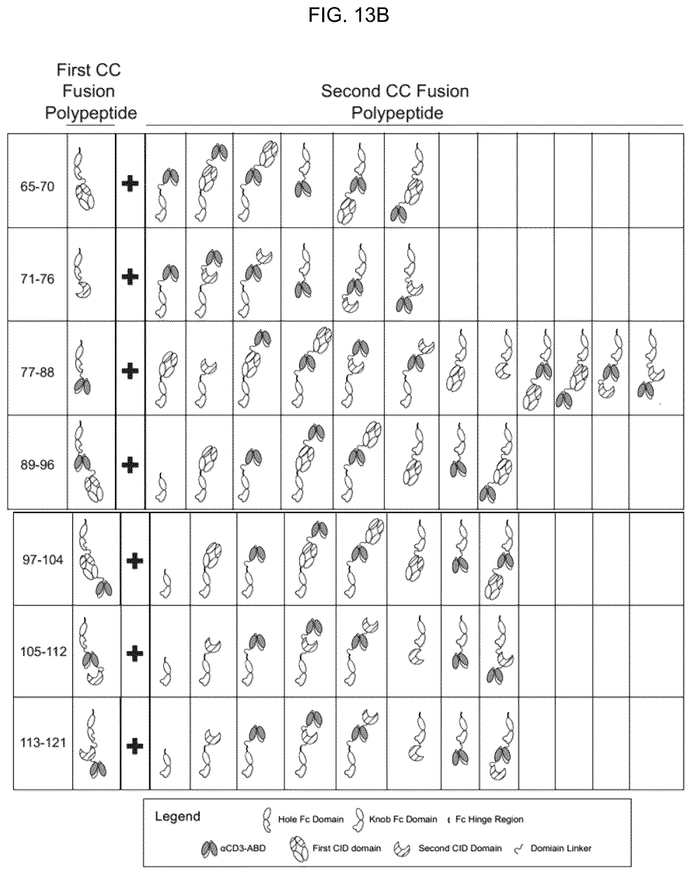

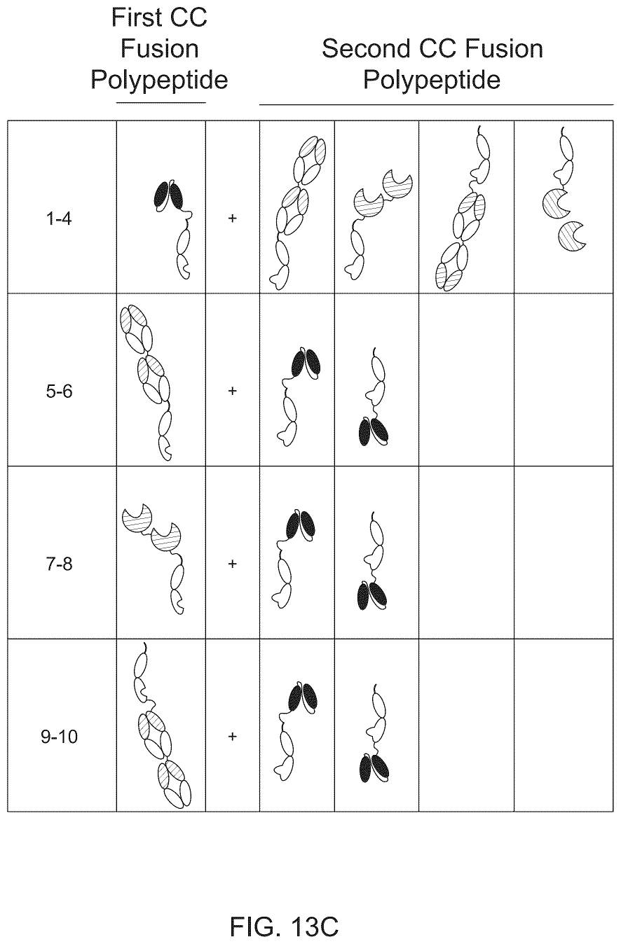

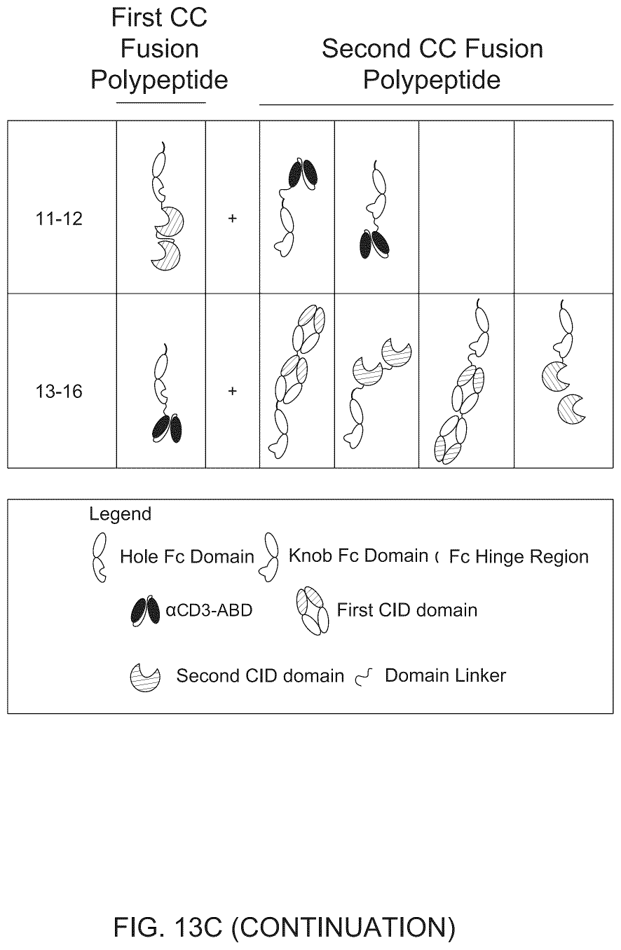

As discussed herein, each of the iCID domains and αCD3 ABD domains of Table 4 can be selected from a Fab, an scFab, an scFvs or a single domain antibody such as the VHH of camelid derived single domain antibody. The Fc domains in the first CC fusion polypeptide and second CC fusion polypeptide heterodimerize with each other. The iCID domain(s) in the first CC fusion polypeptide and/or second CC fusion polypeptide can be selected from either half of the iCID domain pairs described herein. Exemplary formats are illustrated in FIGS. 12A-12G and 13A-13C.

In some embodiments, the CC binding proteins are CC homodimeric binding proteins that use standard Fc domains that self-assemble to form homodimers. In some embodiments, one of either of the iCID domain or the αCD3 ABD is formed using the VH and VL of a traditional, tetrameric antibody, and the other is attached to either the N- or C-terminus of the light chain or the N-terminus of the heavy chain. In some embodiments, one of either of the iCID domain or the αCD3 ABD is formed using the VH and VL of a traditional, tetrameric antibody, and the other is attached to C-terminus of Fc domain. For example, the iCID domain can take a Fab format, and the αCD3 ABD can take an scFv format attached to the C-terminus of the Fc domain. Alternatively, the αCD3 ABD can take a Fab format, and the iCID domain can take an scFv format attached to the C-terminus of the Fc domain. In some embodiments, both the iCID domain and the αCD3 ABD take the format of an scFv or scFab. From N- to C-terminal, the CC binding protein comprises iCID domain-optional domain linker-αCD3 ABD-optional domain linker-homodimeric Fc domain or αCD3 ABD-optional domain linker-iCID domain-optional domain linker-homodimeric Fc domain.

In some instances, the selected arrangements of domains of the dimeric CC binding protein employed in the T-LITE composition provide for an improvement, e.g. in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

(iii) Useful CC Heterodimeric Binding Proteins

As discussed herein, useful CC heterodimeric binding proteins are generally shown in FIGS. 12A-12G and 13A-13C as discussed below. The CC heterodimeric binding protein may comprise a first CC fusion protein and a second CC fusion protein. The first CC fusion protein comprises one or more iCID domain linked to a first heterodimerization Fc domain via an optional domain linker. The second CC fusion protein comprises an αCD3 ABD linked to a second heterodimerization Fc domain via an optional domain linker. The first and second heterodimerization Fc domains heterodimerize to form the CC heterodimeric binding protein. In some embodiments, the first CC fusion protein comprises a plurality of iCIDs. In some embodiments, the first CC fusion protein comprises two iCIDs, for example, as depicted in FIG. 12G. In additional embodiments, each the two iCIDs comprises a indinavir-complex binding domain, for example, including, but not limited to, LS2B described herein.

The iCID domain and αCD3 ABD can take various formats including an Fab, an scFv, an scFab, and a single domain antibody as described herein above. In some embodiments, both the iCID domain and αCD3 ABD take the format of an scFv. In some embodiments, the iCID domain takes the format of an Fab and the αCD3 ABD take the format of an scFv as shown in FIG. 2. In some embodiments, the iCID domain takes the format of an scFab and the αCD3 ABD take the format of an scFv as shown in FIG. 3. In some embodiments, the iCID domain takes the format of a single domain antibody and the αCD3-ABD take the format of an scFv.

In some embodiments, the iCID domain takes the format of a Fab and the αCD3-ABD take the format of an Fab. In some embodiments, the iCID domain takes the format of an scFab and the αCD3 ABD take the format of an Fab. In some embodiments, the iCID domain takes the format of an scFv and the αCD3 ABD take the format of an Fab. In some embodiments, the iCID domain takes the format of a single domain antibody and the αCD3 ABD take the format of a Fab.

In some embodiments, the iCID domain takes the format of an Fab and the αCD3-ABD take the format of an scFab. In some embodiments, the iCID domain takes the format of an scFab and the αCD3 ABD take the format of an scFab. In some embodiments, the iCID domain takes the format of an scFv and the αCD3 ABD take the format of an scFab. In some embodiments, the iCID domain takes the format of a single domain antibody and the αCD3 ABD take the format of an scFab.

In some embodiments, the iCID domain takes the format of an Fab and the αCD3-ABD take the format of a single domain antibody. In some embodiments, the iCID domain takes the format of an scFab and the αCD3 ABD take the format of a single domain antibody. In some embodiments, the iCID domain takes the format of an scFv and the αCD3 ABD take the format of a single domain antibody. In some embodiments, the iCID domain takes the format of a single domain antibody and the αCD3 ABD take the format of a single domain antibody.

In another aspect, the CC heterodimeric binding proteins comprises a Fc fusion protein and an empty Fc domain. The Fc fusion protein comprises one or more iCID domain, an αCD3 ABD, a first heterodimerization Fc domain and one or more optional linkers. The empty Fc domain contains a second heterodimerization Fc domain which heterodimerizes with the first heterodimerization Fc domain. The iCID domain and αCD3 ABD can take various formats including an Fab, an scFv, an scFab, or a single domain antibody as described herein above. In some embodiments, the iCID takes the Fab format, and the αCD3 ABD takes the format of an Fab, an scFv, an scFab, or a single domain antibody. In some embodiments, the iCID takes the scFab format, and the αCD3 ABD takes the format of an Fab, an scFv, an scFab, or a single domain antibody. In some embodiments, the iCID takes the scFv format, and the αCD3 ABD takes the format of an Fab, an scFv, an scFab, or a single domain antibody. In some embodiments, the iCID takes the single domain antibody format, and the αCD3 ABD takes the format of an Fab, an scFv, an scFab, or a single domain antibody. From N to C terminus, the Fc fusion protein can have configurations, such as iCID domain-optional domain linker-αCD3 ABD-optional domain linker-Fc, first iCID domain-optional domain linker-second iCID domain-optional domain linker-αCD3 ABD-optional domain linker-Fc, αCD3 ABD-optional domain linker-iCID domain-optional domain linker-Fc, αCD3 ABD-optional domain linker-first iCID domain-optional domain linker-second iCID domain-optional domain linker-Fc, αCD3 ABD-optional domain linker-Fc-optional domain linker-iCID domain, αCD3 ABD-optional domain linker-Fc-optional domain linker-first iCID domain-optional domain linker-second iCID domain, iCID domain-optional domain linker-Fc-optional domain linker-αCD3 ABD, and first iCID domain-optional domain linker-second iCID domain-optional domain linker-Fc-optional domain linker-αCD3 ABD. Further, from N to C terminus, the Fc fusion protein can have configurations, such as Fc-linker-iCID domain-optional domain linker-CD3 ABD, Fc-optional domain linker-CD3 ABD-optional domain linker-iCID domain, iCID domain-optional domain linker-Fc-liker-CD3 ABD, Fc-optional domain linker-first iCID domain-optional linker-second iCID domain-optional linker-CD3 ABD, Fc-Linker-CD3 ABD-optional domain linker-first iCID domain-optional domain linker-second iCID domain, and first iCID domain-optional domain linker-second iCID domain-optional domain linker-Fc-liker-CD3 ABD. Additionally, from N to C terminus, the Fc fusion protein can have configurations, such as iCID domain-optional domain linker-Fc, Fc-optional domain linker-iCID domain, CD3 ABD-optional domain linker-Fc, Fc-optional domain linker-CD3 ABD, first iCID domain-optional domain linker-Fc-optional domain linker-second iCID domain, first iCID domain-optional domain linker-second iCID domain-optional domain linker-Fc, Fc-optional domain linker-first iCID domain-optional domain linker-second iCID domain, CD3 ABD-optional domain linker-Fc, Fc-optional domain linker-CD3 ABD, first iCID domain-optional domain linker-second iCID domain-optional domain linker-Fc, and Fc-optional domain linker first iCID domain-optional domain linker-second iCID domain. The first and second iCID domains may be identical.

In one aspect, a composition described herein comprises a CC heterodimeric binding protein comprising; i) a first CC fusion protein comprising: 1) a first indinavir chemically induced dimerization (iCID) domain; 2) an optional linker; and 3) a first heterodimerization Fc domain; and ii) a second CC fusion protein comprising: 1) an anti-CD3 antigen binding domain (ABD; αCD3-ABD); 2) an optional domain linker, and 3) a second heterodimerization Fc domain. In some embodiments, the first CC fusion protein may further comprise a second iCID domain linked to the first iCID domain via an optional linker. The first and second iCID domains may be identical.

In one aspect, a composition described herein comprises a monomeric CC binding protein comprising: a) a first indinavir chemically induced dimerization (iCID) domain; b) an optional domain linker; c) an IgG4 monomeric Fc domain; d) an optional domain linker; and e) an anti-CD3 antigen binding domain (ABD; αCD3-ABD). In some embodiments, the monomeric CC binding protein may further comprise a second iCID domain linked to the first iCID domain via an optional linker. The first and second iCID domains may be identical.

In one aspect, a composition described herein comprises a) a first CC fusion protein comprising: 1) a first indinavir chemically induced dimerization (iCID) domain; 2) an optional domain linker; 3) an αCD3-ABD; and 4) a first heterodimerization Fc domain; and ii) a second CC fusion protein comprising: 1) a second heterodimerization Fc domain. In some embodiments, the first CC fusion protein may further comprise a second iCID domain linked to the first iCID domain via an optional linker. The first and second iCID domains may be identical.

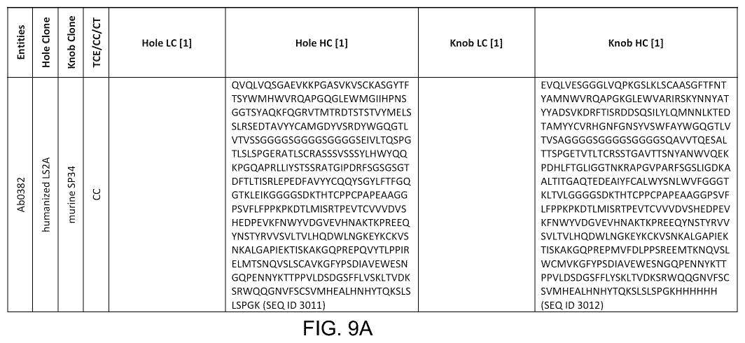

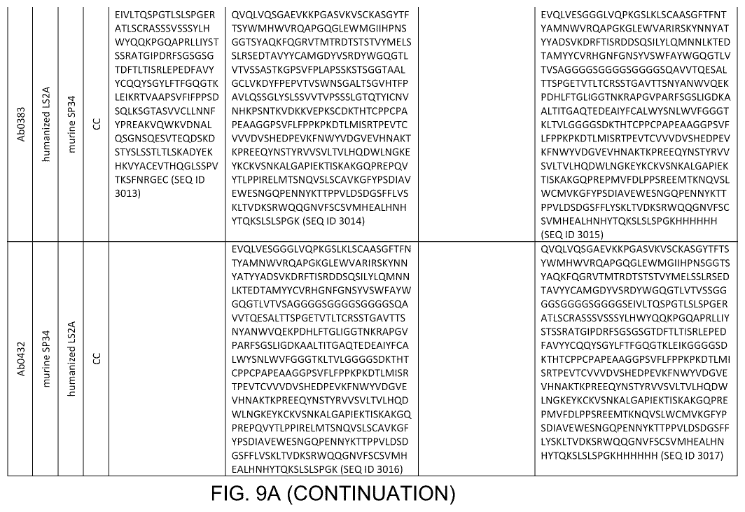

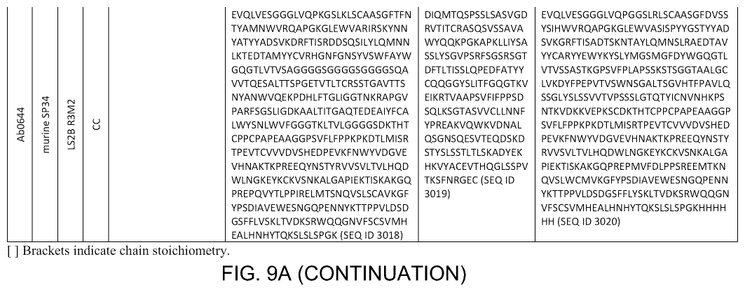

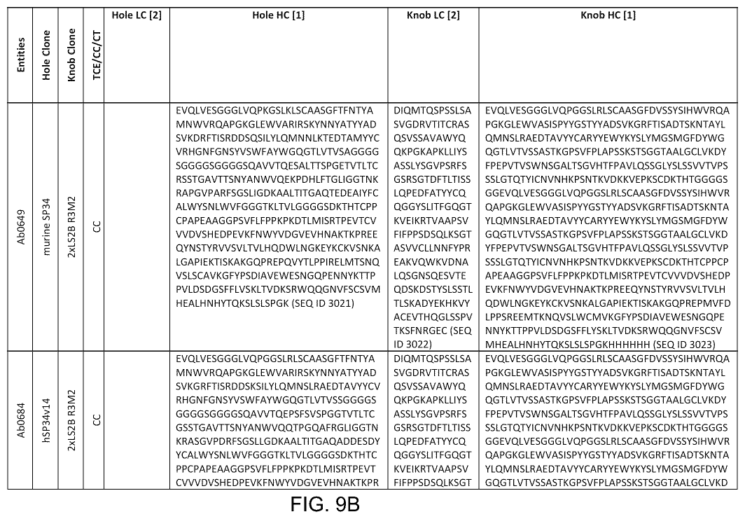

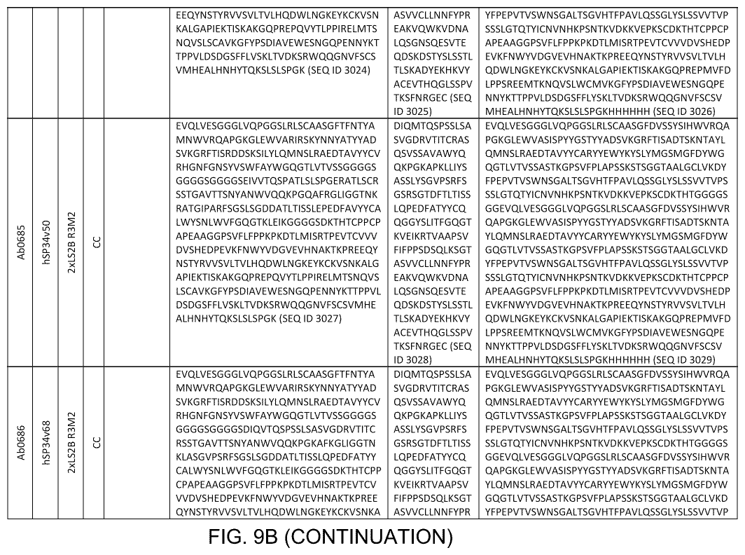

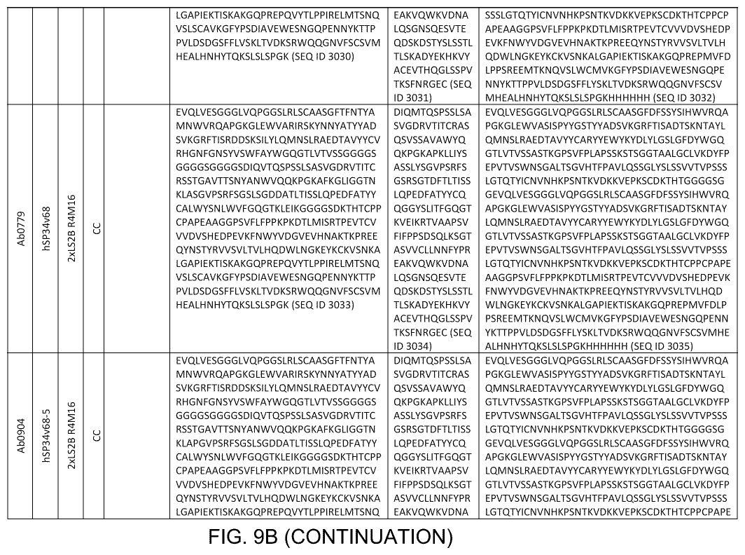

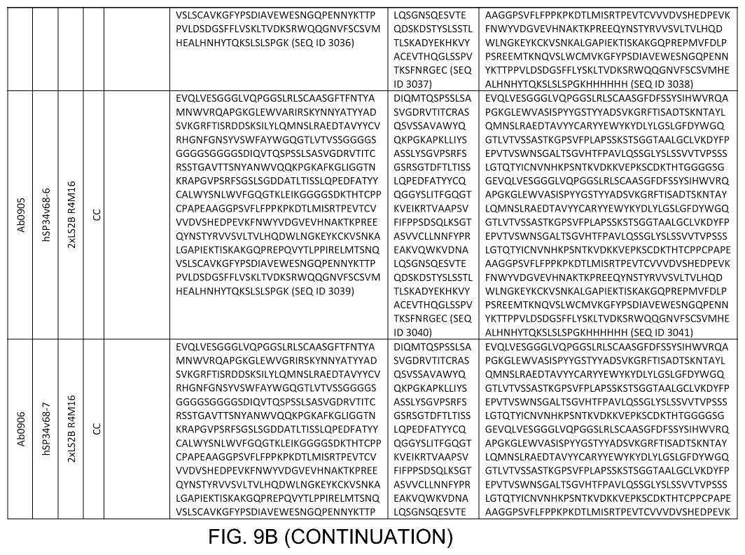

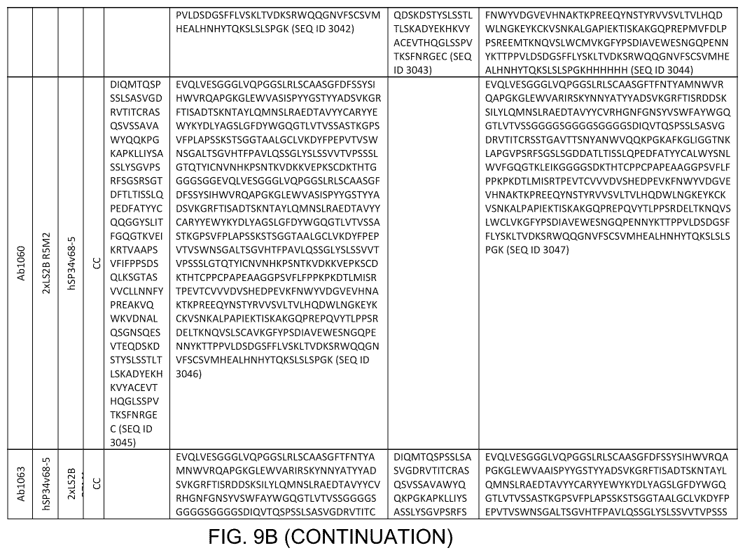

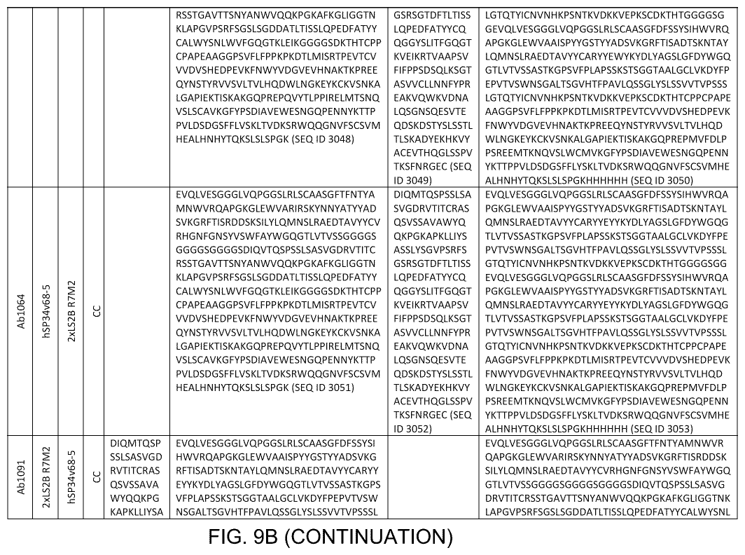

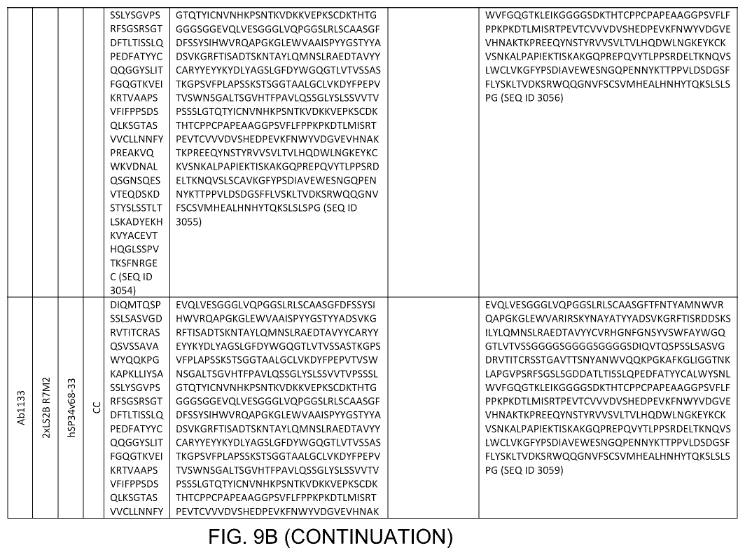

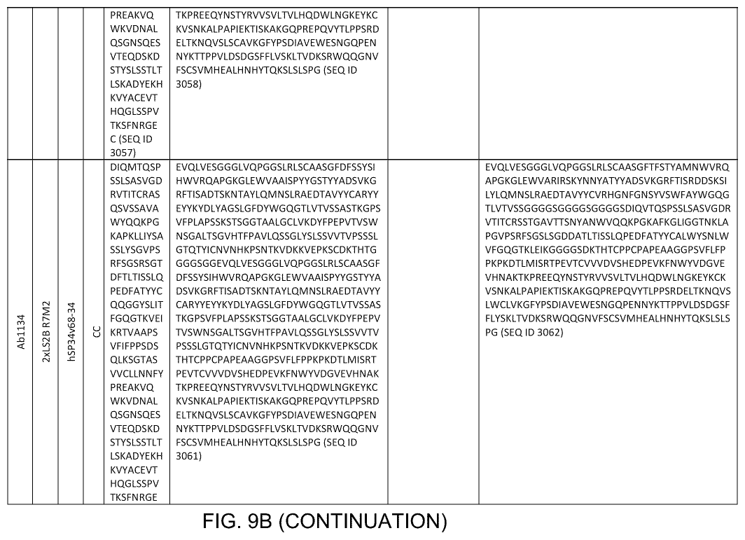

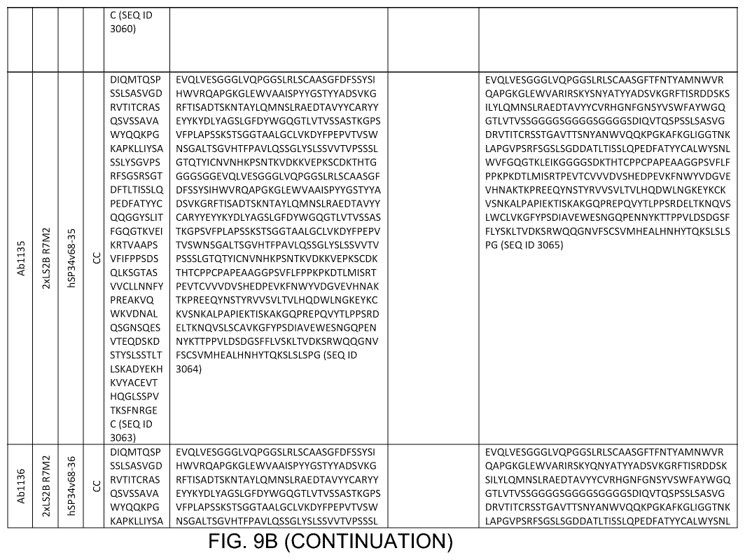

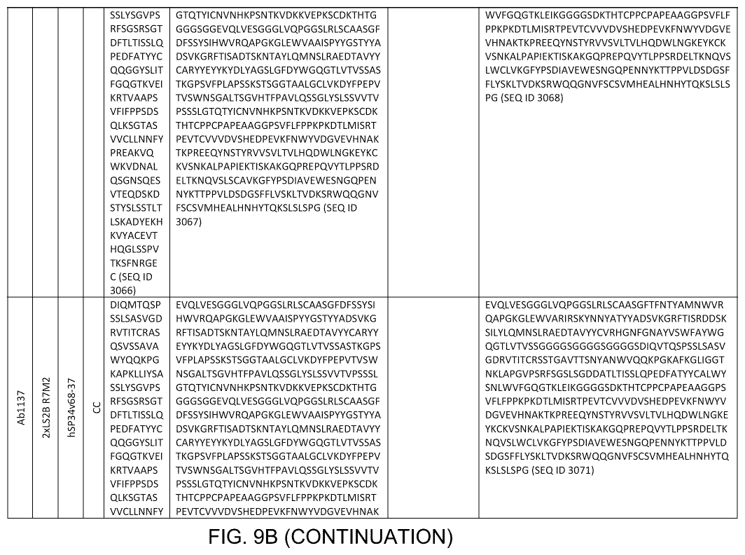

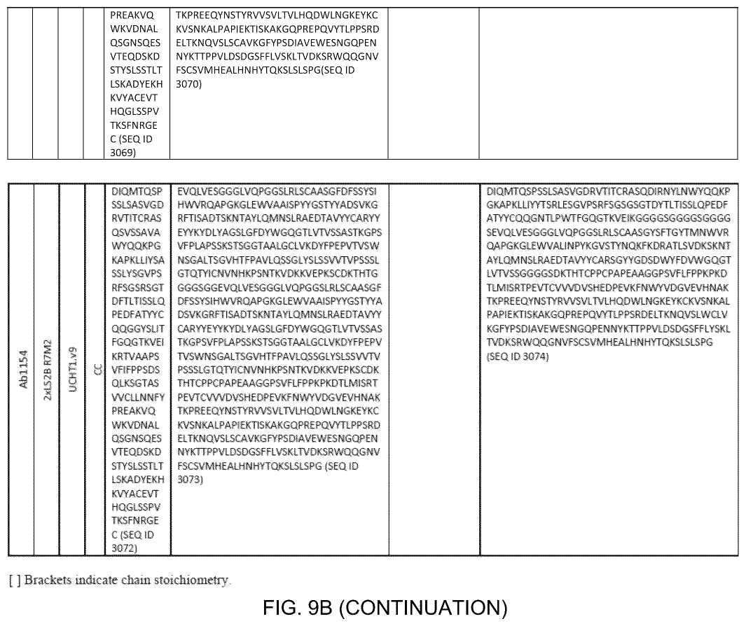

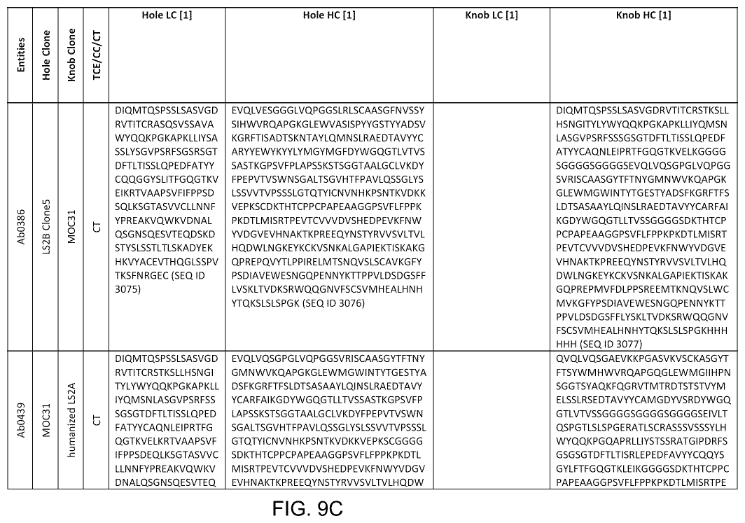

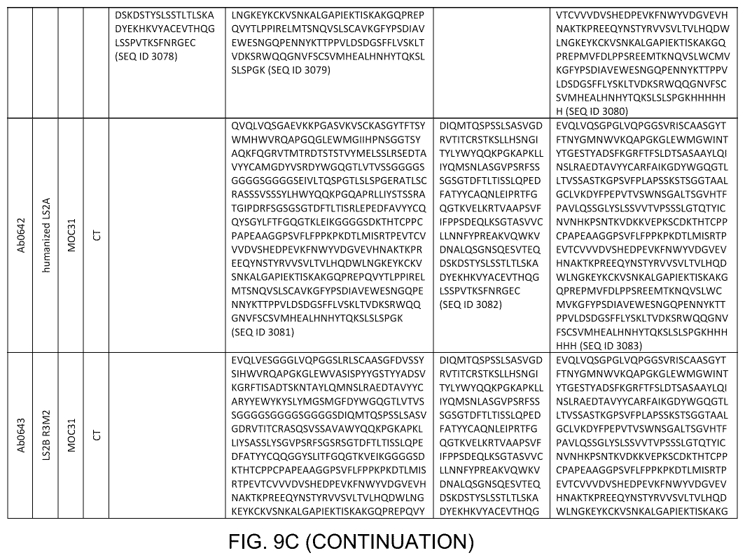

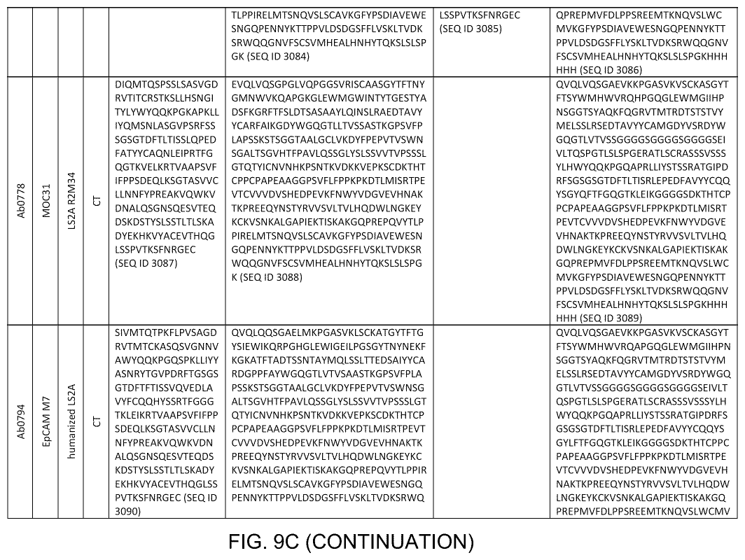

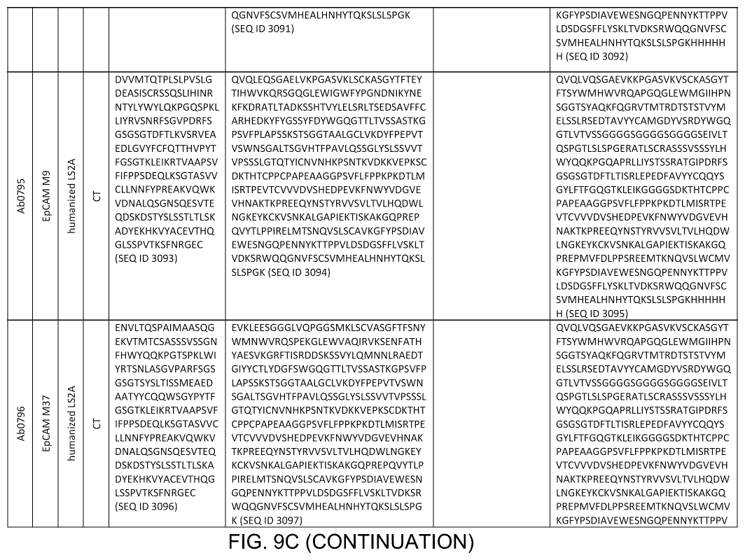

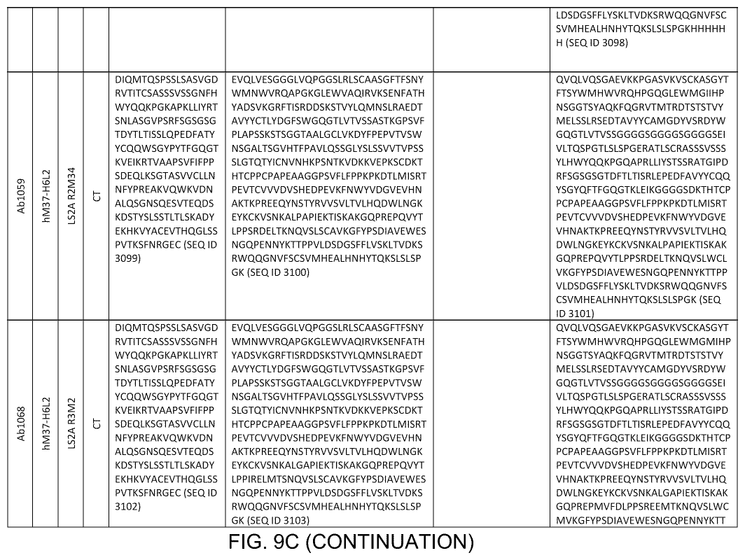

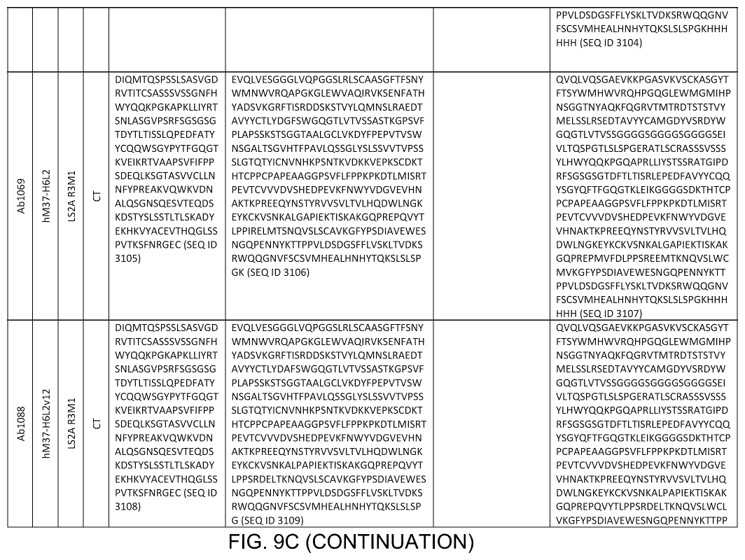

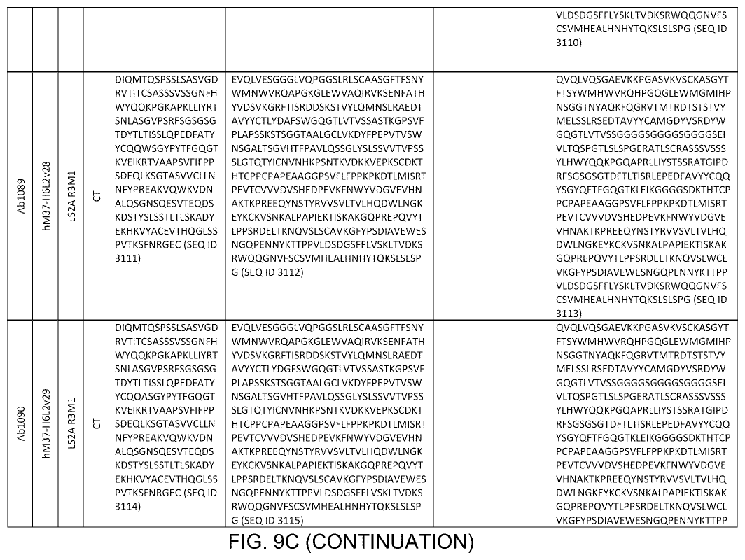

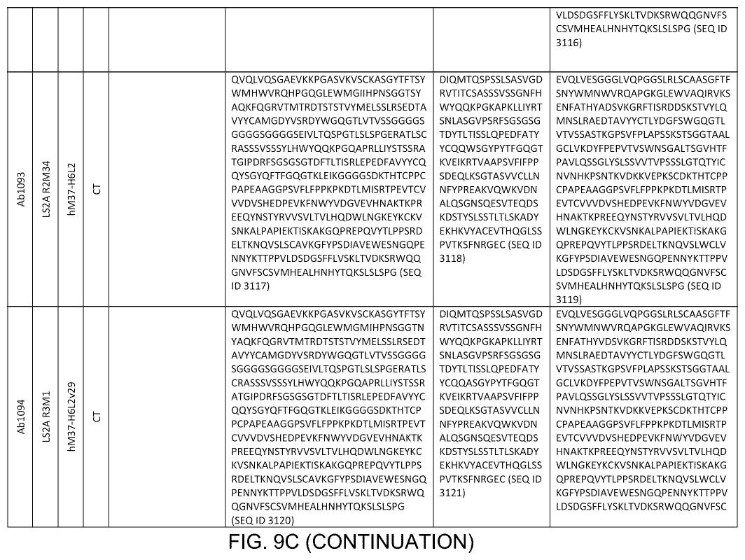

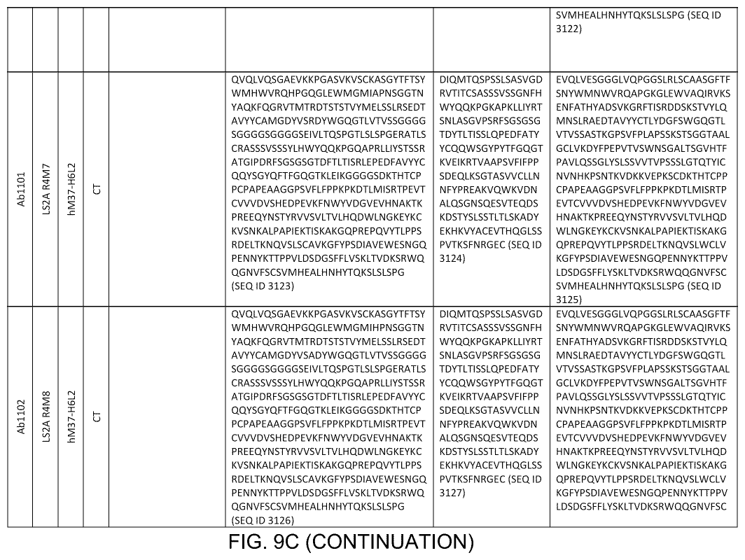

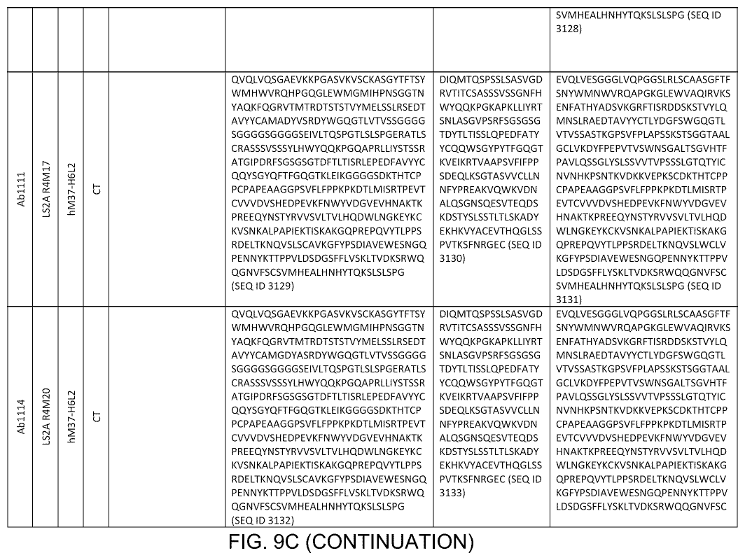

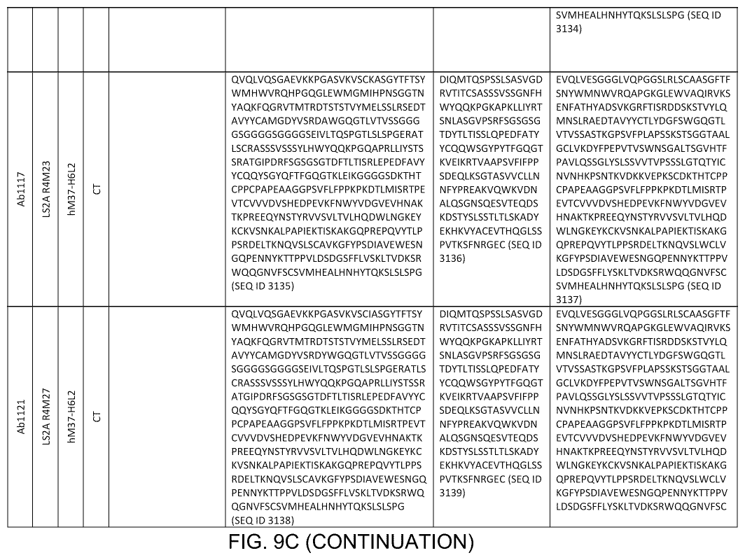

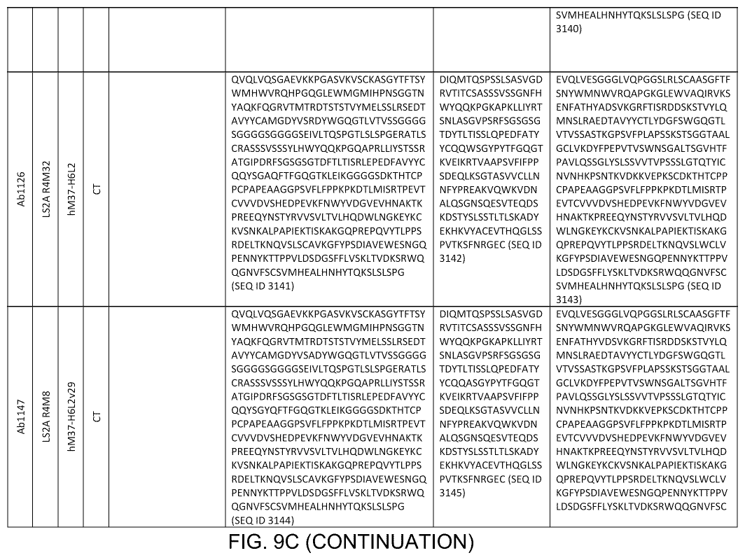

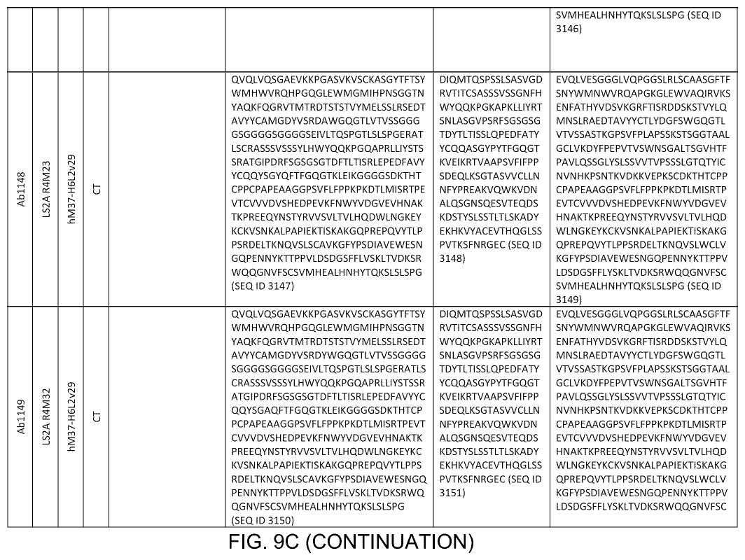

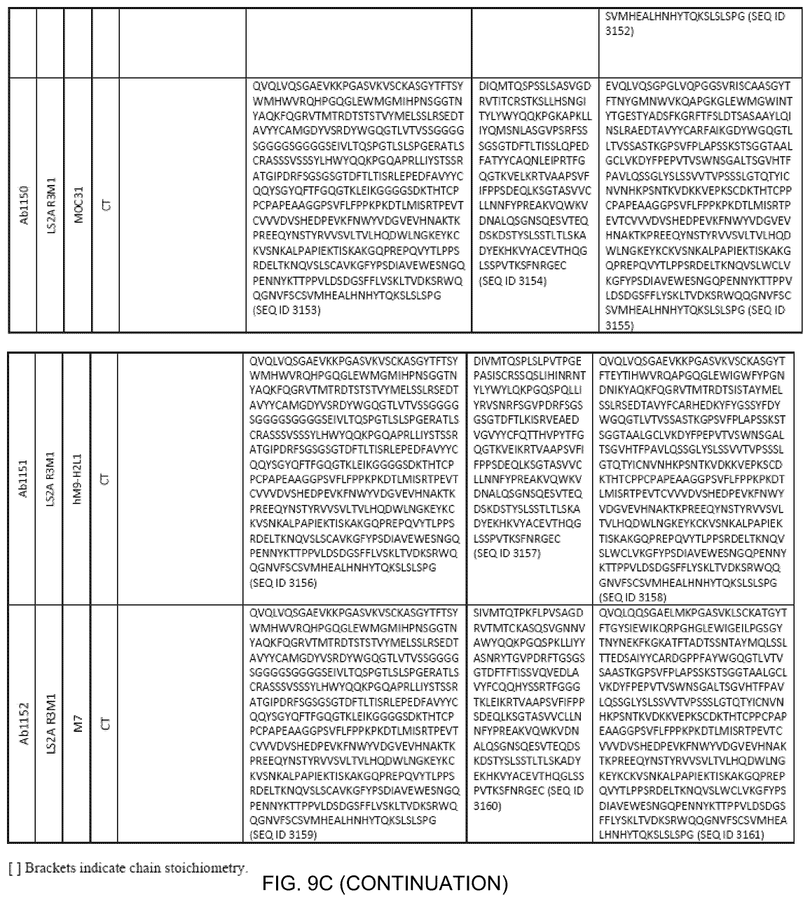

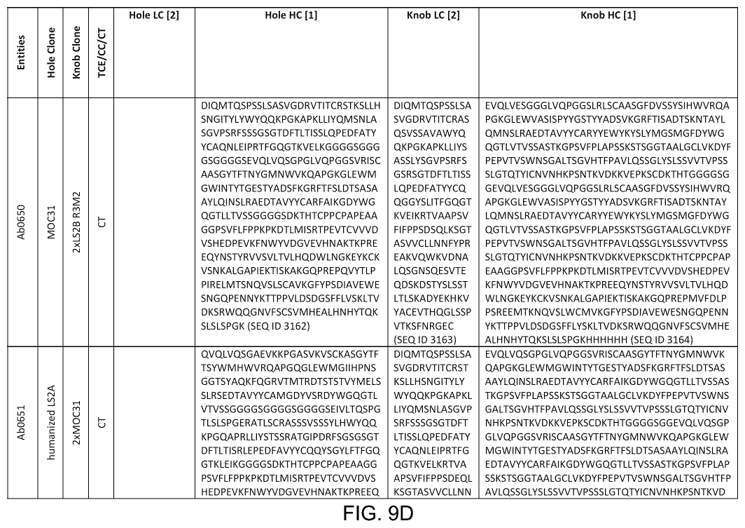

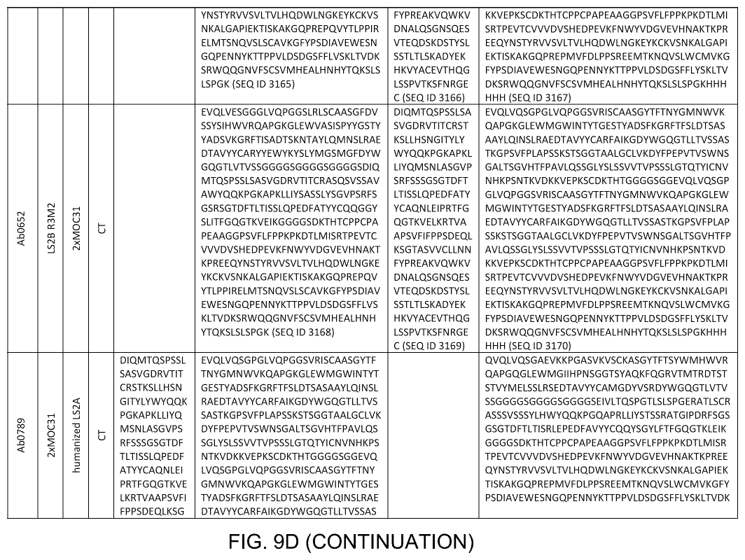

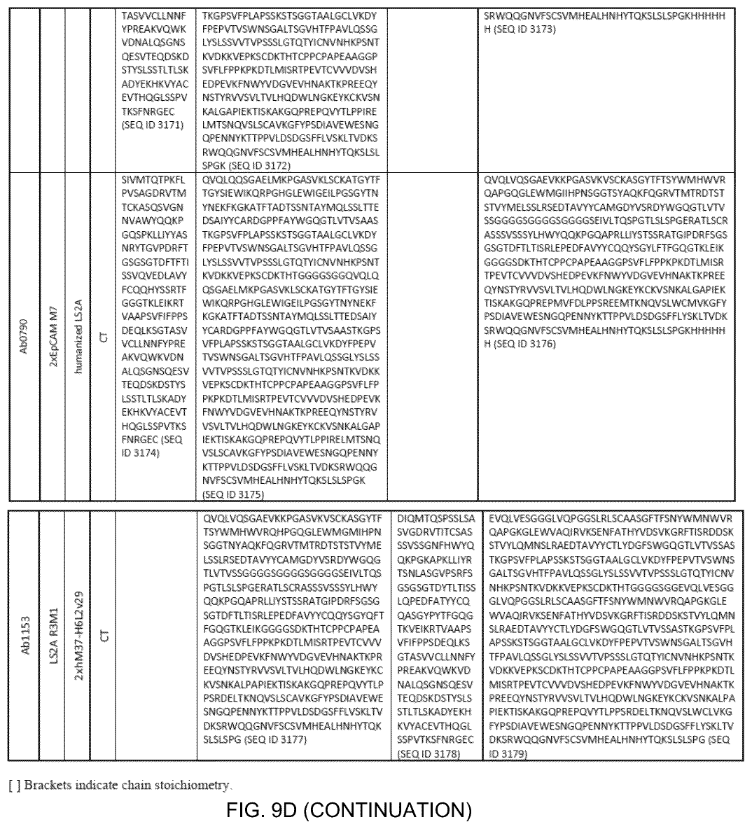

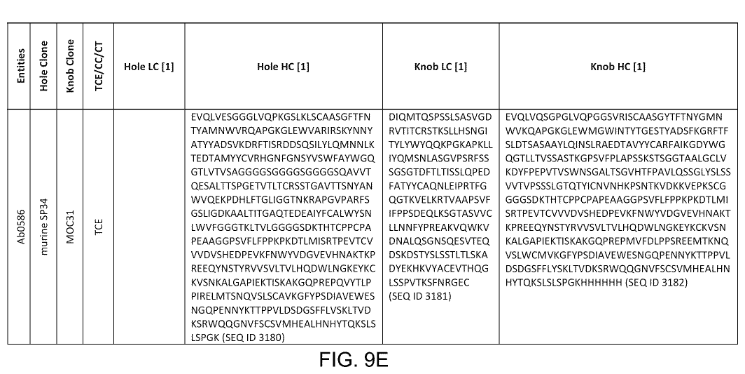

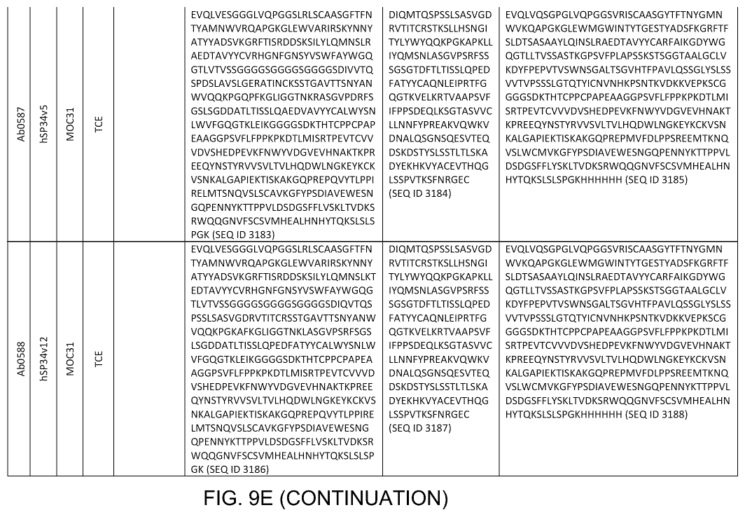

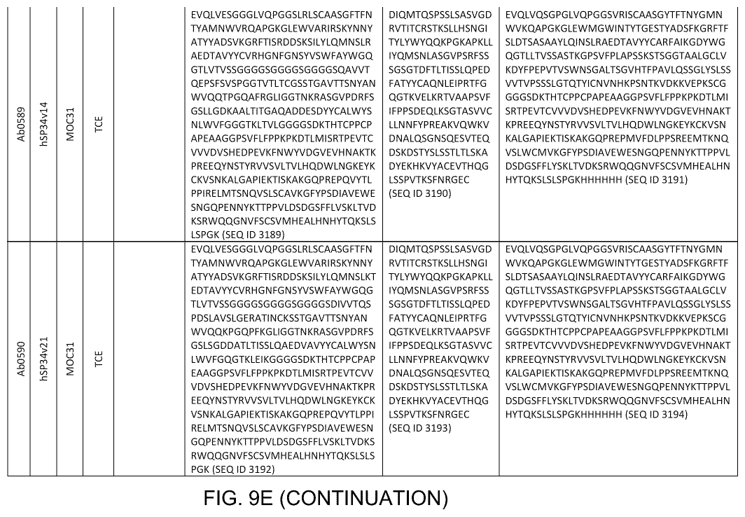

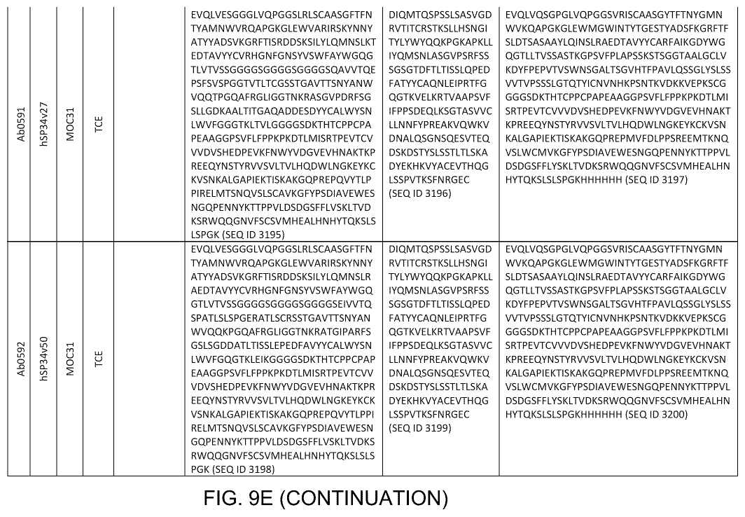

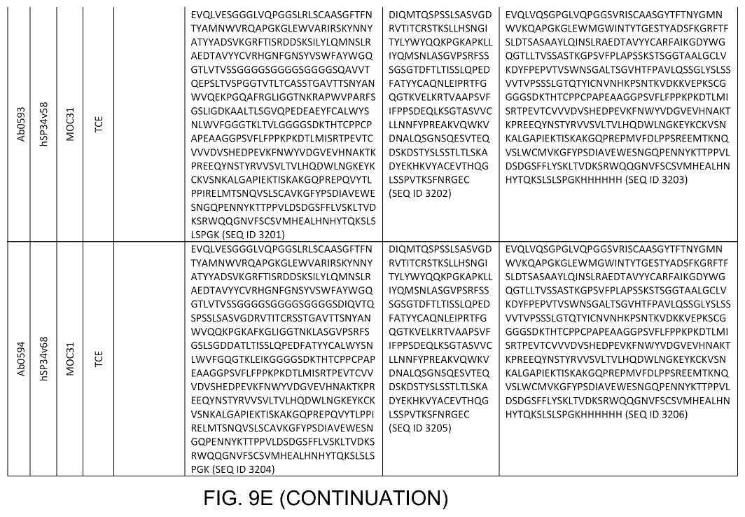

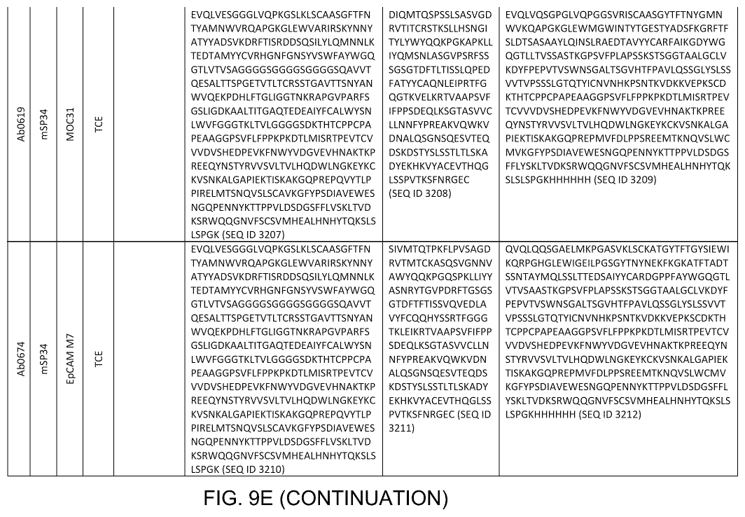

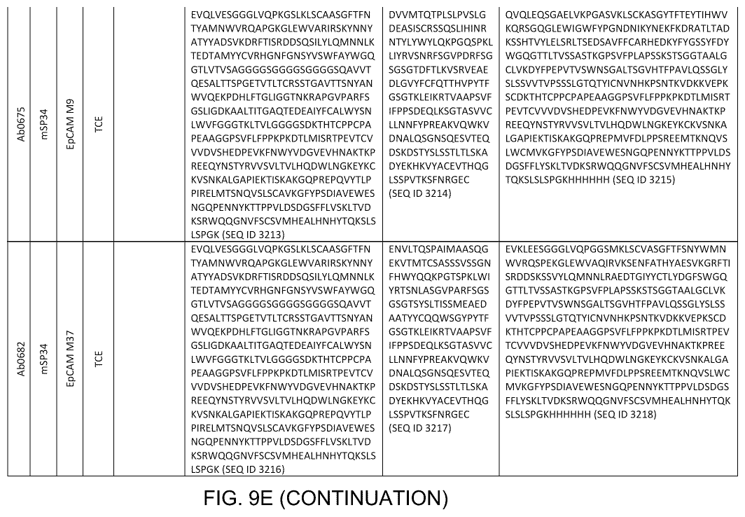

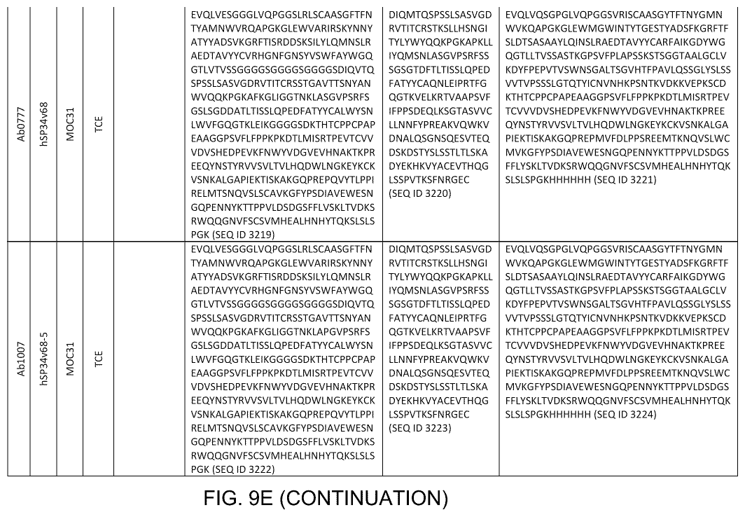

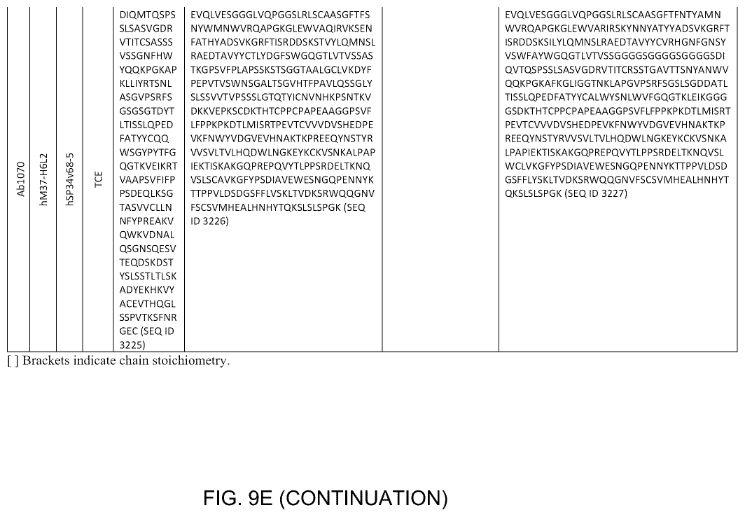

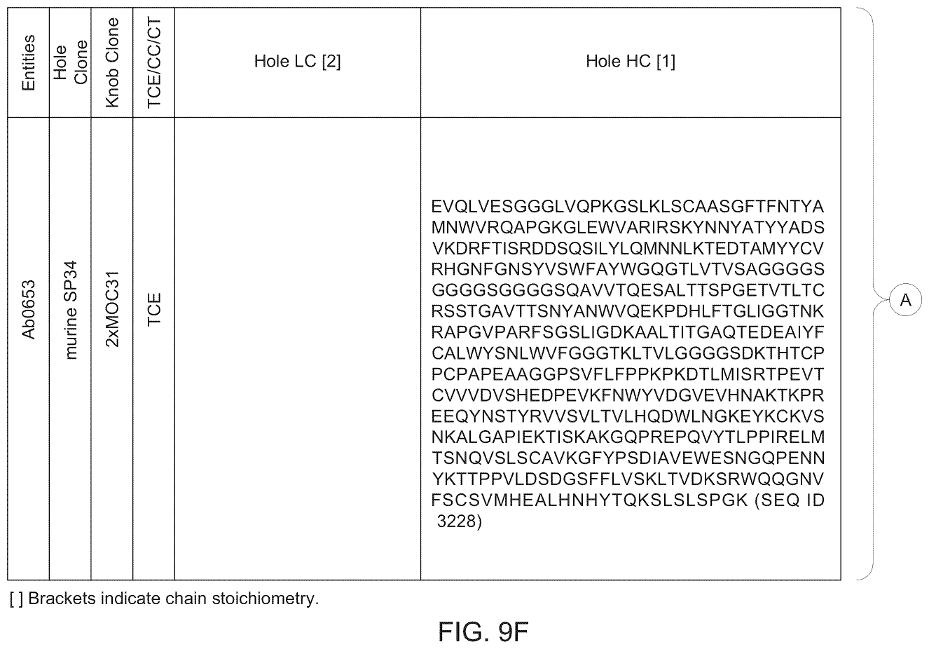

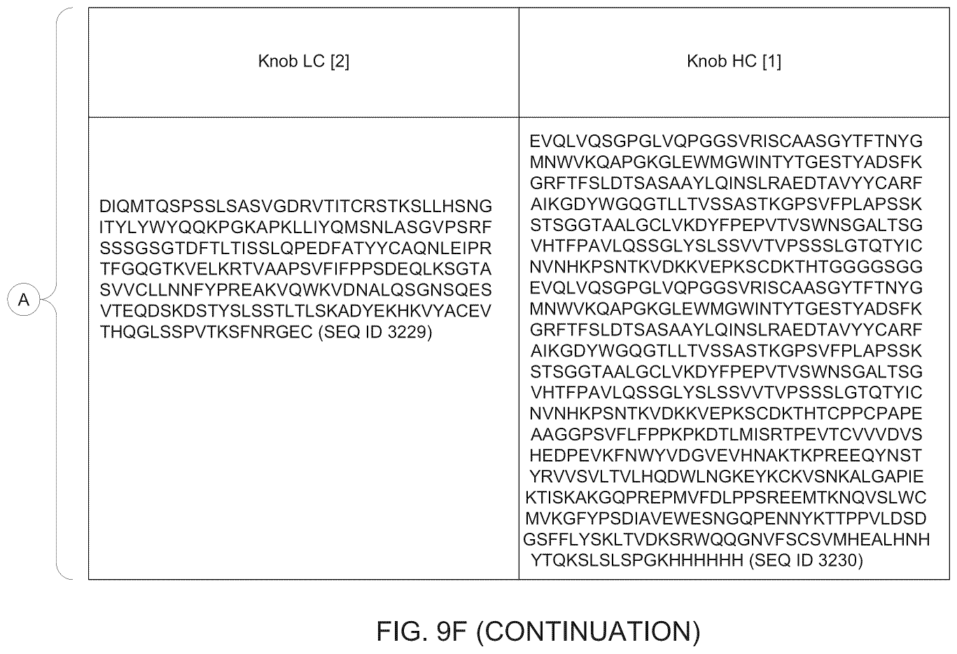

In some embodiments, the first iCID domain may comprise a composition comprising an indinavir binding domain. In some embodiments, the first iCID domain may comprise a composition comprising an indinavir-complex binding domain. In some embodiments, the composition comprises one or more heavy chain and/or light chain sequences selected from the group consisting of heavy chain and/or light chain sequences from FIGS. 9A and 9B. In some embodiments, the composition comprises any one of CC heterodimeric binding proteins of FIGS. 9A and 9B. FIG. 9A depicts exemplary CC heterodimeric binding proteins with one iCID domain. In some embodiments, the composition comprises Ab0382 of FIG. 9A. In some embodiments, the composition comprises Ab00383 of FIG. 9A. In some embodiments, the composition comprises Ab0432 of FIG. 9A. In some embodiments, the composition comprises Ab0644 of FIG. 9A. FIG. 9B depicts exemplary CC heterodimeric binding proteins with two iCID domains, each comprising an indinavir binding domain. In some embodiments, the composition comprises Ab0649 of FIG. 9B. In some embodiments, the composition comprises Ab0684 of FIG. 9B. In some embodiments, the composition comprises Ab0685 of FIG. 9B. In some embodiments, the composition comprises Ab0686 of FIG. 9B. In some embodiments, the composition comprises Ab0779 of FIG. 9B. In some embodiments, the composition comprises Ab0904 of FIG. 9B. In some embodiments, the composition comprises Ab0905 of FIG. 9B. In some embodiments, the composition comprises Ab0906 of FIG. 9B. In some embodiments, the composition comprises Ab1060 of FIG. 9B. In some embodiments, the composition comprises Ab1063 of FIG. 9B. In some embodiments, the composition comprises Ab1064 of FIG. 9B. In some embodiments, the composition comprises Ab1091 of FIG. 9B. In some embodiments, the composition comprises Ab1133 of FIG. 9B. In some embodiments, the composition comprises Ab1134 of FIG. 9B. In some embodiments, the composition comprises Ab1135 of FIG. 9B. In some embodiments, the composition comprises Ab1136 of FIG. 9B. In some embodiments, the composition comprises Ab1137 of FIG. 9B.

In another aspect, said first iCID domain is an indinavir binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4-D11, P7-D12, P1-C11, P4-F9, P6-HS, P2-D7, P3-FS, P3-C5, P8-F4, P8-C2, P7-F12, P2-H7, P1-B7, P2-D9, P6-E8, P3-B9, P1-G1, P1-D11, P8-E3, P4-E4, PS-D12, P3-F4, P6-H2, P1-E9, P8-D9, and P4-G11 (FIG. 50). In another aspect, said first iCID domain is an indinavir binding domain comprising a VH domain and VL domain selected from the group consisting of the VH and VL domains of P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1- B12, P4-G12, P2-A10, P4-D11, P7-D12, P1-C11, P4-F9, P6-HS, P2-D7, P3-FS, P3-C5, P8-F4, P8-C2, P7-F12, P2-H7, P1-B7, P2-D9, P6-E8, P3-B9, P1-G1, P1-D11, P8-E3, P4-E4, PS-D12, P3-F4, P6-H2, P1-E9, P8-D9, and P4-G11 (FIG. 50). In another aspect, said first iCID is an indinavir-complex binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). In another aspect, said first iCID is an indinavir-complex binding domain comprising a VH domain and VL domain selected from the group consisting of the VH and VL domains of Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50).

b. CT Binding Proteins

Similar to CC binding proteins, the invention provides CT binding proteins. Each CT binding protein contains an iCID domain, and an anti-TTABD (αTTABD). In some embodiments, CT binding proteins do not contain an Fc domain, such as direct fusion of an iCID domain and an αTTABD. Both the iCID domain and the αTTABD can take the format of an scFv, a Fab, an scFab or a single domain antibody such as the VHH of camelid derived single domain antibody. In some embodiments, CT binding proteins contain an Fc domain. In some cases, CT binding proteins rely on the use of a monomeric Fc domain, such that the CT binding proteins are monomeric, as more fully outlined below. In some embodiments, CT binding proteins comprise a first and a second CT fusion polypeptide, that come together as dimers, either heterodimerically or homodimerically, to provide the αTTAABD functionally coupled to an iCID domain.

### (i) Monomeric CT Fusion Polypeptides

Accordingly, when the CT binding protein relies on the use of a monomeric IgG4 Fc domain, the CT binding protein is a fusion polypeptide with a structure selected from the group, from N- to C-terminal: iCID domain-optional domain linker-αTTABD-optional domain linker-Fc domain; iCID domain-optional domain linker-first αTTABD-optional domain linker-second αTTABD-optional domain linker-Fc domain; αTTABD-optional domain linker-iCID domain-optional domain linker-Fc domain; first αTTABD-optional domain linker-second αTTABD-optional domain linker-iCID domain-optional domain linker-Fc domain; iCID domain-optional domain linker-Fc domain-optional domain linker-αTTABD; iCID domain-optional domain linker-Fc domain-optional domain linker-first αTTABD-optional domain linker-second αTTABD; αTTABD-optional domain linker-iCID domain-optional domain linker-Fc domain; first αTTABD-optional domain linker-second αTTABD-optional domain linker-iCID domain-optional domain linker-Fc domain; Fc domain-optional domain linker-αTAABD-optional domain linker-iCID domain; and Fc domain-optional domain linker-first αTAABD-optional domain linker-second αTAABD-optional domain linker-iCID domain and Fc domain-optional domain linker-iCID domain-optional domain linker-αTTABD. Either or both of the iCID and αTTABD can take any one of the formats including a Fab, an scFv, an scFab, and a single domain antibody such as the VHH of camelid derived single domain antibody. The first and second αTTABDs may be identical. In some embodiments, the first and second αTTABDs are different.

In some instances, the selected arrangements of domains of the monomeric CT binding protein employed in the T-LITE composition provide an improvement, e.g., in synthesis, stability, affinity or effector activity, over other structures disclosed herein or known in the art. In some cases, a 2-fold, 3-fold, or 4-fold increase in, e.g. synthesis, stability, affinity, or effector function is observed. In some cases, a 5-fold, 6-fold, 7-fold, 8-fold, 9-fold, 10-fold or higher increase in, e.g. stability, affinity, or effector function, is observed.

### (ii) Dimeric CT Binding Proteins

In some embodiments, the CT binding protein comprises a first and a second CT fusion polypeptide, that come together as dimers, either heterodimerically or homodimerically, to provide the αTAABD functionally coupled to a CID domain. In these embodiments, the CT binding proteins rely on the use of Fc domains that are dimers, either heterodimeric Fc domains or homodimeric Fc domains.

In some embodiments, the CT binding proteins are CT heterodimeric binding proteins that use heterodimerization variants in the Fc domains. Thus, in some embodiments, the CT binding protein comprises a first and a second CT fusion polypeptide, wherein one of the first and second CT fusion polypeptides contains the αTAABD and the other the iCID domain. In some embodiments, the CT binding protein comprises a first CT fusion polypeptide which contains both the αTAABD and the iCID domain, and a second CT fusion polypeptide comprising an empty Fc domain. In these embodiments, the first and second CT fusion polypeptides can have the structures (from N- to C-terminal, with “DL” standing for “domain linker”) as shown in Table 5. In some embodiments, each of the CT fusion polypeptide having one αTAABD may further comprise another αTAABD linked to the αTAABD via an optional DL, for example, as shown in 37-40 in Table 4 below. The iCID domains in the same fusion polypeptide may be identical. In some embodiments, the iCID domains in the same fusion polypeptide may be different. In some embodiments, each of the CT fusion polypeptide having one iCID domains may further comprise another iCID domain linked to the iCID domain via an optional DL, for example, as shown in 41-44 in Table 4 below. The iCID domains in the same fusion polypeptide may be identical. In some embodiments, the iCID domains in the same fusion polypeptide may be different. The DLs in the same fusion polypeptide may be different. In some embodiments, the DLs in the same fusion polypeptide may be same.

TABLE 5

| | First CT fusion polypeptide | Second CT fusion polypeptide |
|---|---|---|
| | (N- to C- terminal) | (N- to C- terminal) |
| 1 | Fc domain | iCID-DL-Fc domain |
| 2 | Fc domain | αTAABD-DL-Fc domain |
| 3 | Fc domain | αTAABD-DL-iCID-DL-Fc domain |
| 4 | Fc domain | iCID-DL-αTAABD-DL-Fc domain |
| 5 | Fc domain | Fc domain-DL-iCID |
| 6 | Fc domain | Fc domain-DL-αTAABD |
| 7 | Fc domain | Fc domain-DL-αTAABD-DL-iCID |
| 8 | Fc domain | Fc domain-DL-iCID-DL-αTAABD |
| 9 | iCID-DL-Fc domain | αTAABD-DL-Fc domain |
| 10 | iCID-DL-Fc domain | αTAABD-DL-iCID-DL-Fc domain |
| 11 | iCID-DL-Fc domain | iCID-DL-αTAABD-DL-Fc domain |
| 12 | iCID-DL-Fc domain | Fc domain-DL-iCID |
| 13 | iCID-DL-Fc domain | Fc domain-DL-αTAABD |
| 14 | iCID-DL-Fc domain | Fc domain-DL-αTAABD-DL-iCID |
| 15 | iCID-DL-Fc domain | Fc domain-DL-iCID-DL-αTAABD |
| 16 | αTAABD-DL-Fc domain | αTAABD-DL-iCID-DL-Fc domain |
| 17 | αTAABD-DL-Fc domain | iCID-DL-αTAABD-DL-Fc domain |
| 18 | αTAABD-DL-Fc domain | Fc domain-DL-iCID |
| 19 | αTAABD-DL-Fc domain | Fc domain-DL-αTAABD |
| 20 | αTAABD-DL-Fc domain | Fc domain-DL-αTAABD-DL-iCID |
| 21 | αTAABD-DL-Fc domain | Fc domain-DL-iCID-DL-αTAABD |
| 22 | αTAABD-DL-iCID-DL-Fc domain | iCID-DL-αTAABD-DL-Fc domain |
| 23 | αTAABD-DL-iCID-DL-Fc domain | Fc domain-DL-iCID |
| 24 | αTAABD-DL-iCID-DL-Fc domain | Fc domain-DL-αTAABD |
| 25 | αTAABD-DL-iCID-DL-Fc domain | Fc domain-DL-αTAABD-DL-iCID |
| 26 | αTAABD-DL-iCID-DL-Fc domain | Fc domain-DL-iCID-DL-αTAABD |
| 27 | iCID-DL-αTAABD-DL-Fc domain | Fc domain-DL-iCID |
| 28 | iCID-DL-αTAABD-DL-Fc domain | Fc domain-DL-αTAABD |
| 29 | iCID-DL-αTAABD-DL-Fc domain | Fc domain-DL-αTAABD-DL-iCID |
| 30 | iCID-DL-αTAABD-DL-Fc domain | Fc domain-DL-iCID-DL-αTAABD |
| 31 | Fc domain-DL-iCID | Fc domain-DL-αTAABD |
| 32 | Fc domain-DL-iCID | Fc domain-DL-αTAABD-DL-iCID |
| 33 | Fc domain-DL-iCID | Fc domain-DL-iCID-DL-αTAABD |
| 34 | Fc domain-DL-αTAABD | Fc domain-DL-αTAABD-DL-iCID |
| 35 | Fc domain-DL-αTAABD | Fc domain-DL-iCID-DL-αTAABD |
| 36 | Fc domain-DL-αTAABD-DL-iCID | Fc domain-DL-iCID-DL-αTAABD |
| 37 | iCID-DL- Fc domain | αTAABD -DL-αTAABD-DL-Fc domain |
| 38 | iCID-DL- Fc domain | Fc domain-DL- αTAABD-DL- αTAABD |
| 39 | Fc domain-DL-iCID | αTAABD -DL-αTAABD-DL-Fc domain |
| 40 | Fc domain-DL-iCID | Fc domain-DL- αTAABD-DL- αTAABD |
| 41 | iCID-DL-iCID-DL-Fc domain | αTAABD -DL-Fc domain |
| 42 | iCID-DL-iCID-DL-Fc domain | Fc domain-DL- αTAABD |
| 43 | Fc domain-DL-iCID-DL-iCID | αTAABD -DL-Fc domain |
| 44 | Fc domain-DL-iCID-DL-iCID | Fc domain-DL-αTAABD |
| 45 | iCID-DL-iCID-DL-Fc domain | αTAABD -DL-αTAABD-DL-Fc domain |
| 46 | iCID-DL-iCID-DL-Fc domain | Fc domain-DL- αTAABD-DL- αTAABD |
| 47 | Fc domain-DL-iCID-DL-iCID | αTAABD -DL-αTAABD-DL-Fc domain |
| 48 | Fc domain-DL-iCID-DL-iCID | Fc domain-DL- αTAABD-DL- αTAABD |
| 49 | iCID-DL-Fc domain | αTAABD-DL- αTAABD-DL Fc domain |
| 50 | iCID-DL-Fc domain | αTAABD-DL- αTAABD-DL iCID-DL- |
| | | Fc domain |
| 51 | iCID-DL-Fc domain | iCID-DL-αTAABD-DL- αTAABD-DL |
| | | Fc domain |
| 53 | iCID-DL-Fc domain | Fc domain-DL-αTAABD-DL-αTAABD |
| 54 | iCID-DL-Fc domain | Fc domain-DL-αTAABD-DL-αTAABD - |
| | | DL-iCID |
| 55 | iCID-DL-Fc domain | Fc domain-DL-iCID-DL-αTAABD-DL- |
| | | αTAABD |
| 56 | Fc domain-DL-iCID-DL-iCID | αTAABD -DL- αTAABD -DL-Fc |
| | | domain |
| 57 | Fc domain-DL-iCID-DL-iCID | Fc domain-DL-αTAABD-DL-αTAABD |
| 58 | Fc domain-DL-iCID | Fc domain-DL-αTAABD-DL-αTAABD |
| 59 | Fc domain-DL-iCID | Fc domain-DL-αTAABD-DL-αTAABD - |
| | | DL-iCID |
| 60 | Fc domain-DL-iCID | Fc domain-DL-iCID-DL-αTAABD-DL- |
| | | αTAABD |

In some embodiments, the first CC fusion polypeptide may be iCID-DL-iCID-DL-Fc domain. In some embodiments, the first CC fusion polypeptide may be αTAABD-DL-iCID-DL-iCID-DL-Fc domain. In some embodiments, the first CC fusion polypeptide may be iCID-DL-iCID-DL-αTAABD-DL-Fc domain. In some embodiments, the first CC fusion polypeptide may be Fc domain-DL-iCID-DL-iCID. In some embodiments, the first CC fusion polypeptide may be Fc domain-DL-αTAABD-DL-iCID-DL-iCID.

In some embodiments, the second CC fusion polypeptide may be iCID-DL-iCID-DL-Fc domain. In some embodiments, the second CC fusion polypeptide may be αCD3-ABD-DL-iCID-DL-iCID-DL-Fc domain. In some embodiments, the second CC fusion polypeptide may be iCID-DL-iCID-DL-αCD3-ABD-DL-Fc domain. In some embodiments, the second CC fusion polypeptide may be Fc domain-DL-iCID-DL-iCID. In some embodiments, the second CC fusion polypeptide may be Fc domain-DL-αCD3-ABD-DL-iCID-DL-iCID. In some embodiments, the second CC fusion polypeptide may be Fc domain-DL-iCID-DL-iCID-DL-αCD3-ABD.

As discussed herein, each of the iCID domains and αTAABD domains of Table 5 can be selected from a Fab, an scFab, an scFvs or a single domain antibody such as the VHH of camelid derived single domain antibody. The Fc domains in the first CT fusion polypeptide and second CT fusion polypeptide heterodimerize with each other. The iCID domain(s) in the first CT fusion polypeptide and/or second CT fusion polypeptide can be selected from either half of the iCID domain pairs described herein.

In some instances, the selected arrangements of domains of the dimeric CT binding protein employed in the T-LITE composition provide an improvement, e.g. in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

In some embodiments, the CT binding proteins are CT homodimeric binding proteins that use standard Fc domains that self-assemble to form homodimers. In some embodiments, one of either of the iCID domain or the αTTABD is formed using the VH and VL of a traditional, tetrameric antibody, and the other is attached to either the N- or C-terminus of the light chain or the C-terminus of the heavy chain. In some embodiments, the iCID domain takes a Fab format, and the αTTABD takes an scFv format. Alternatively, the αTTABD can take a Fab format, and the iCID domain can take an scFv format attached to either the N- or C-terminus of the light chain or the C-terminus of the heavy chain of the αTTABD.

In some embodiments, one of either of the iCID domain or the αTTABD is formed using the VH and VL of a traditional, tetrameric antibody, and the other is attached to C-terminus of Fc domain. In some embodiments, the iCID domain takes a Fab format, and the αTTABD takes an scFv format. Alternatively, the αTTABD can take a Fab format, and the iCID domain can take an scFv format attached to the C-terminus of the Fc domain.

In some embodiments, both the iCID domain and the αTTABD take the format of an scFv or scFab. From N- to C-terminal, the CT binding protein comprises iCID domain-αTTABD-homodimeric Fc domain or αTTABD-iCID domain-homodimeric Fc domain.

(iii) Useful CT Heterodimeric Binding Proteins

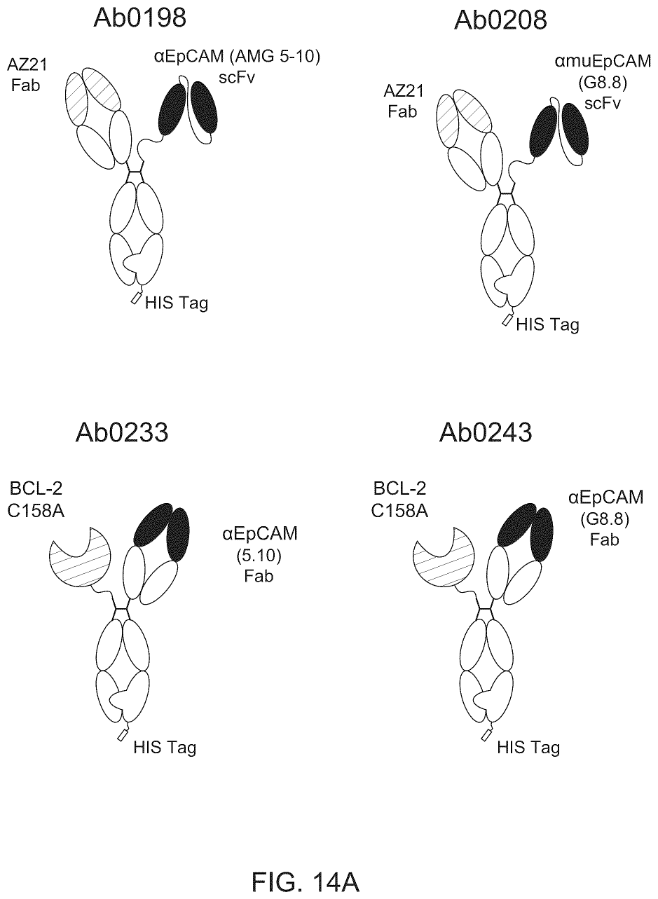

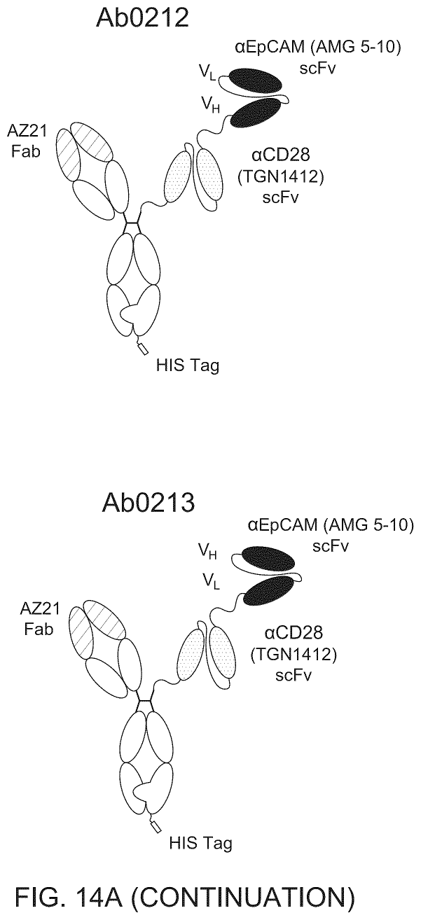

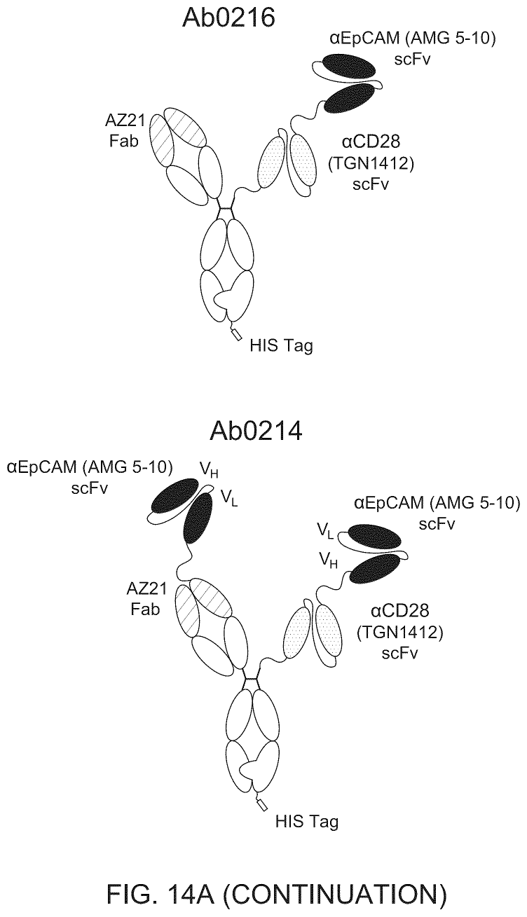

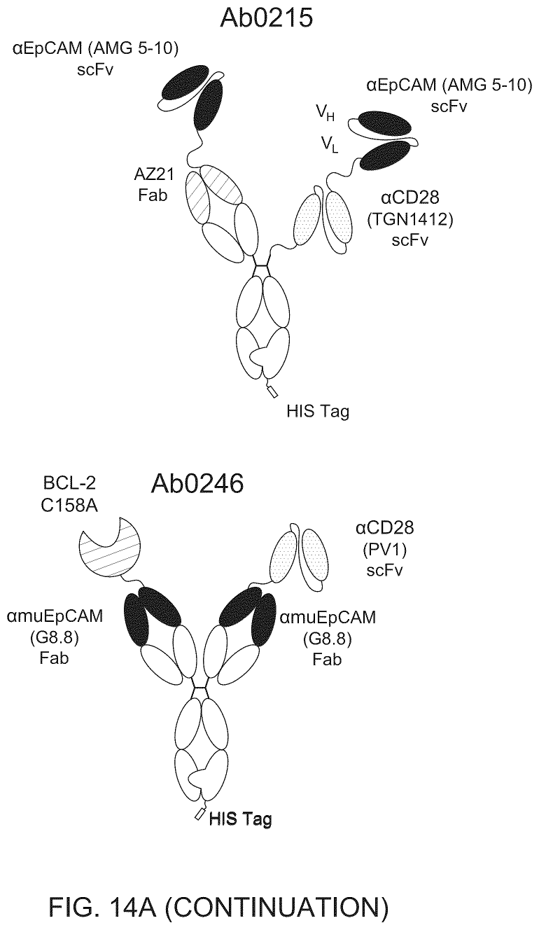

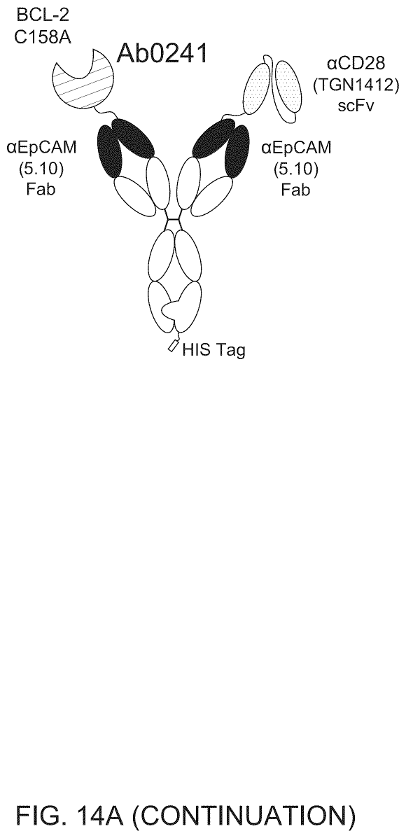

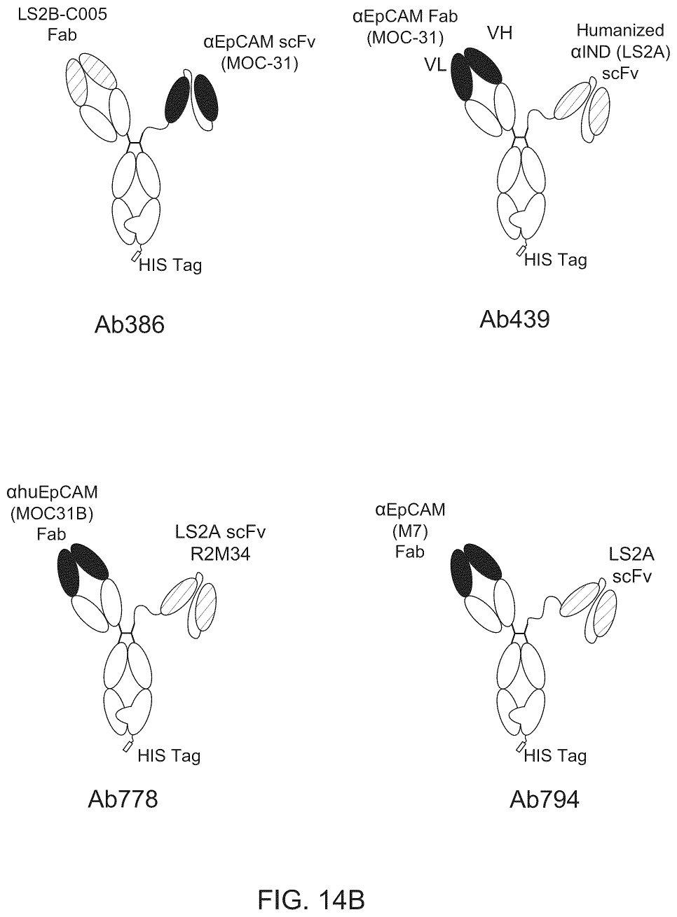

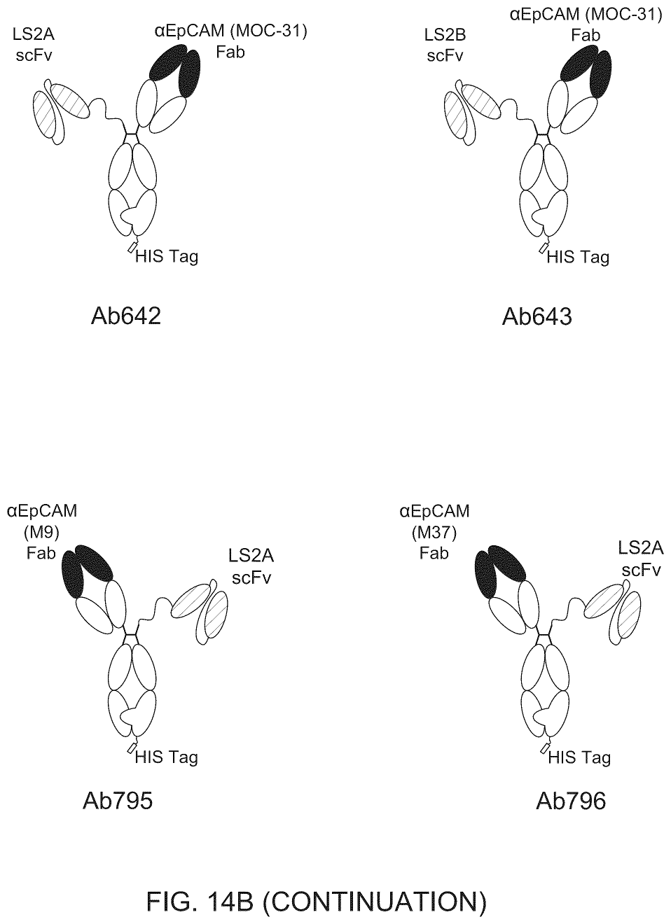

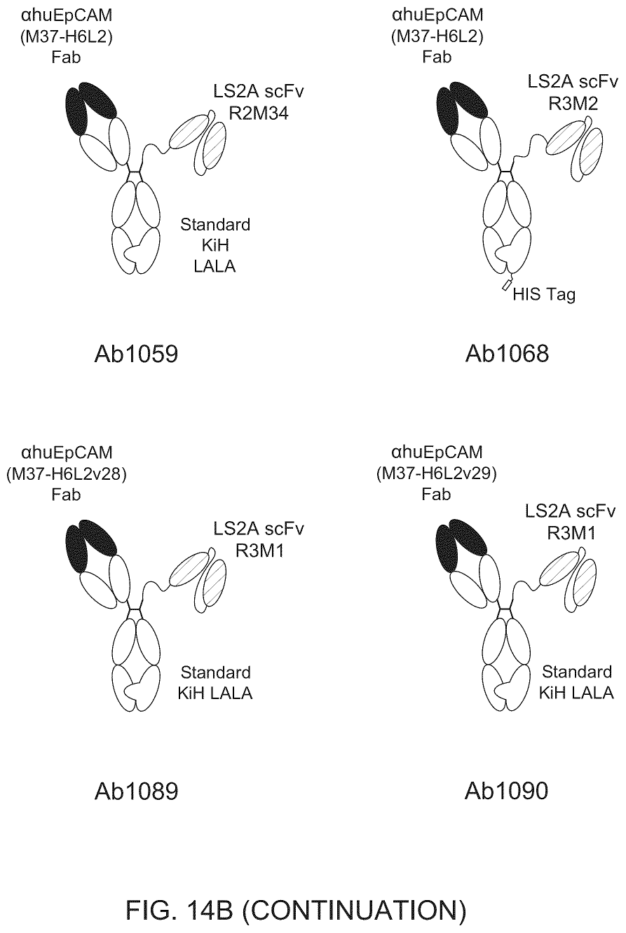

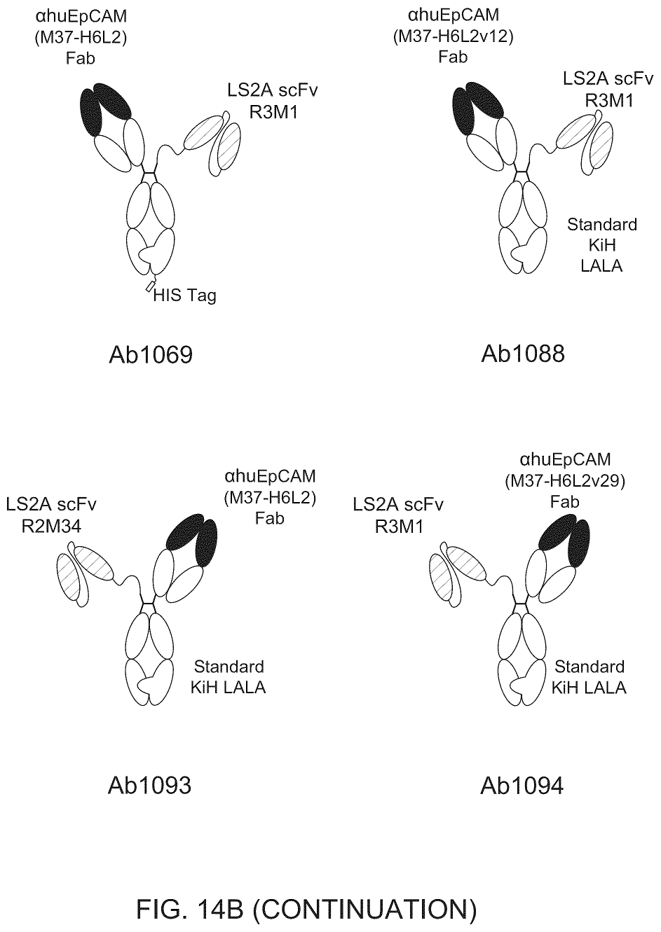

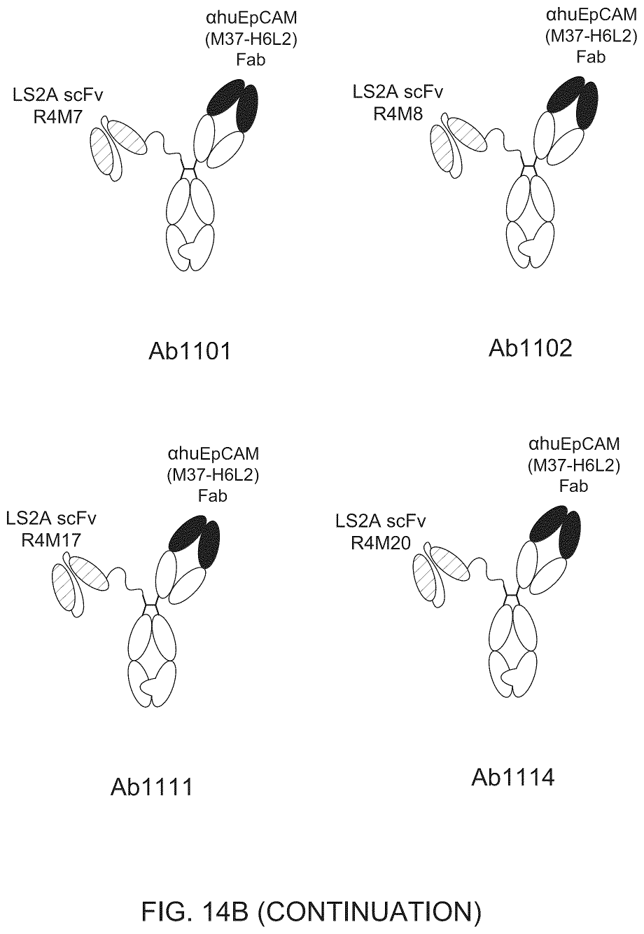

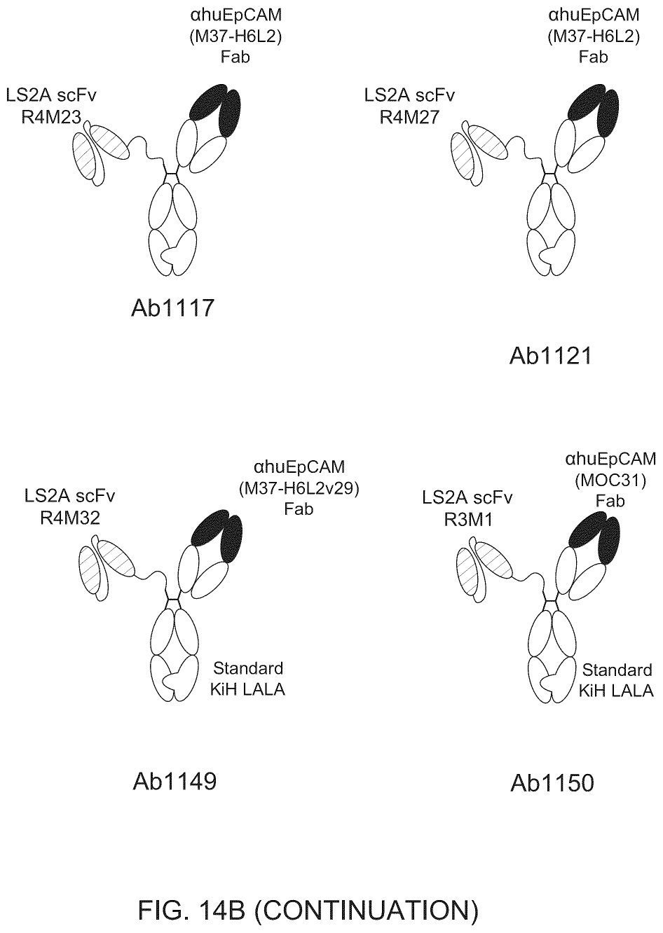

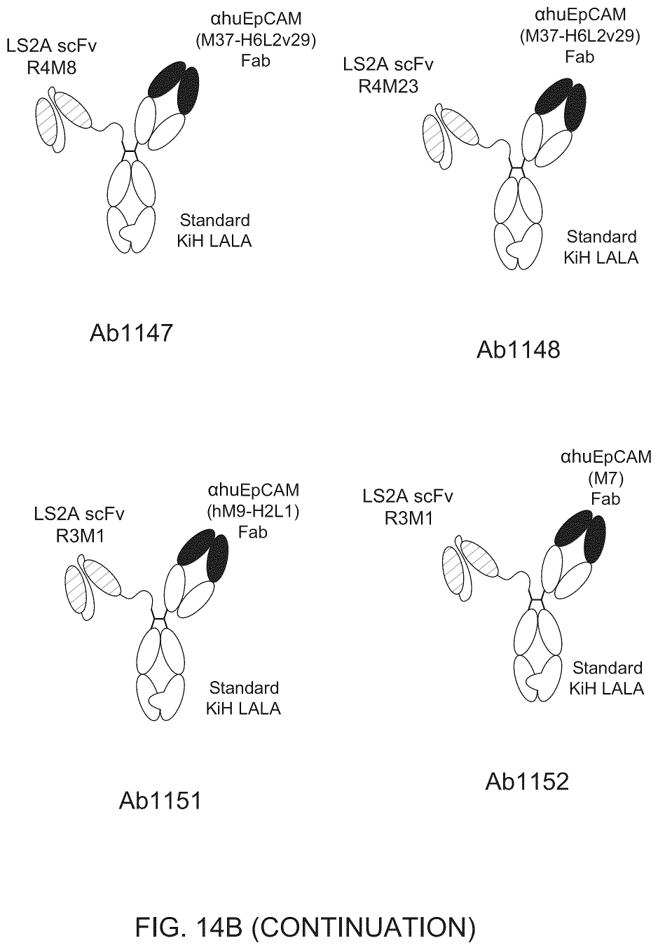

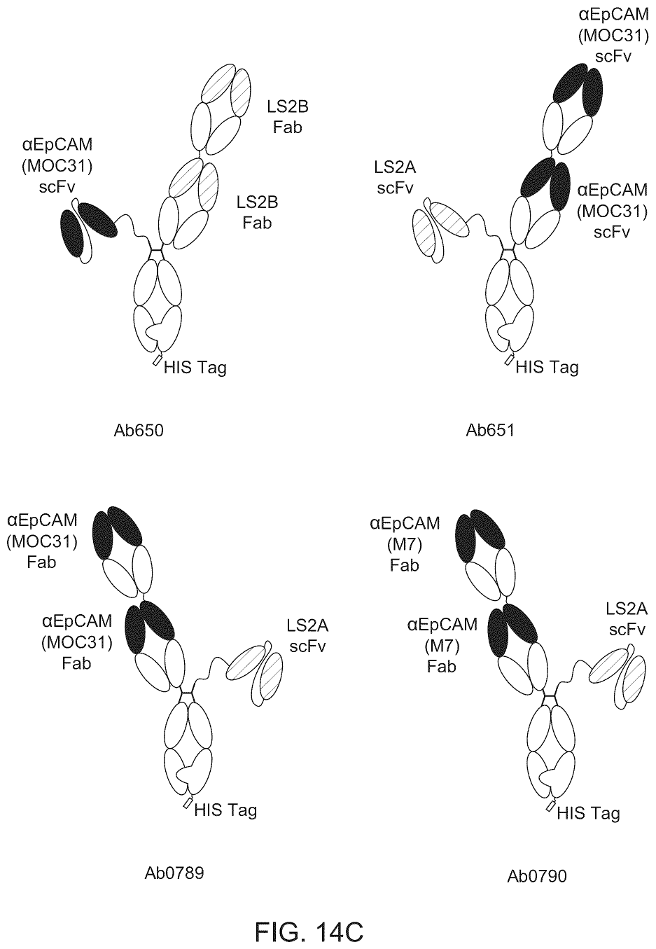

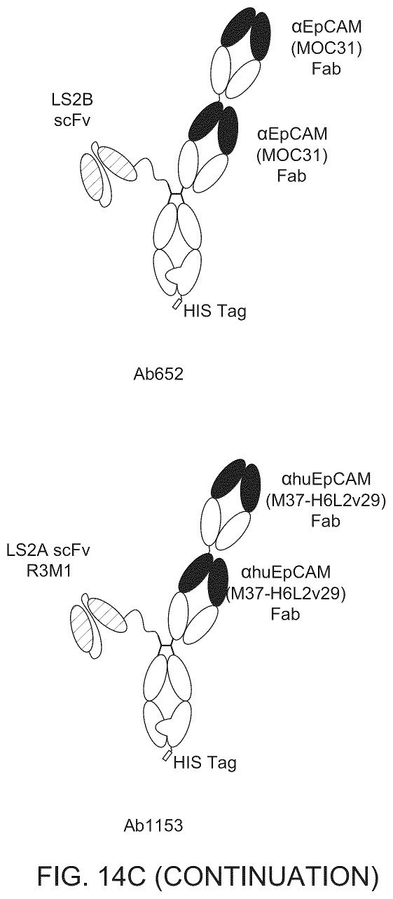

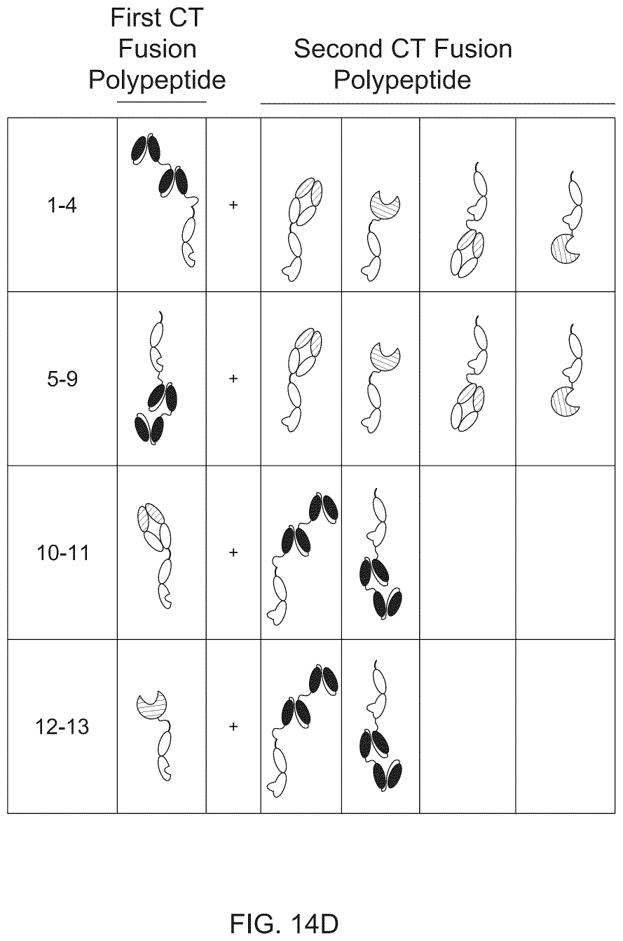

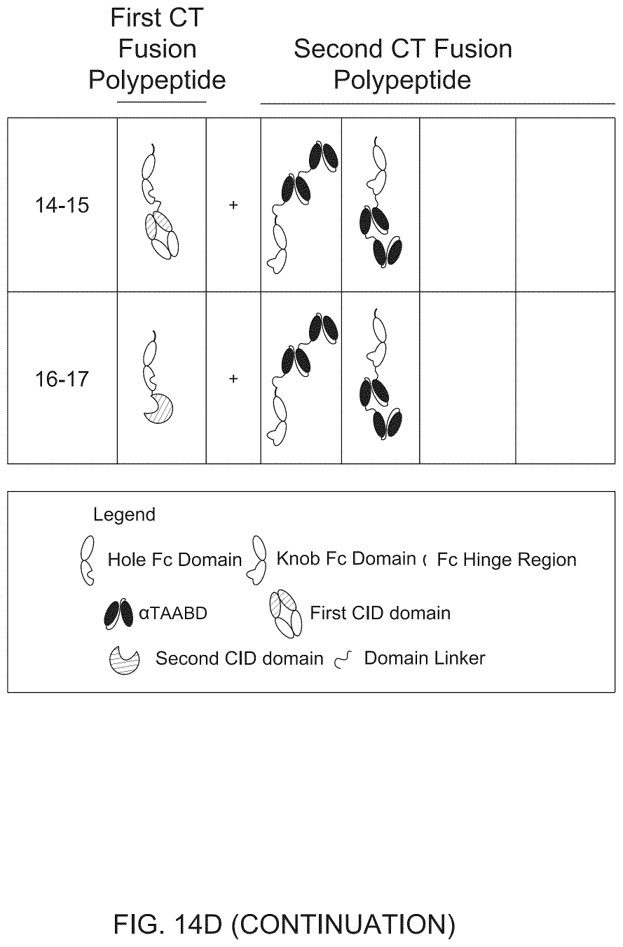

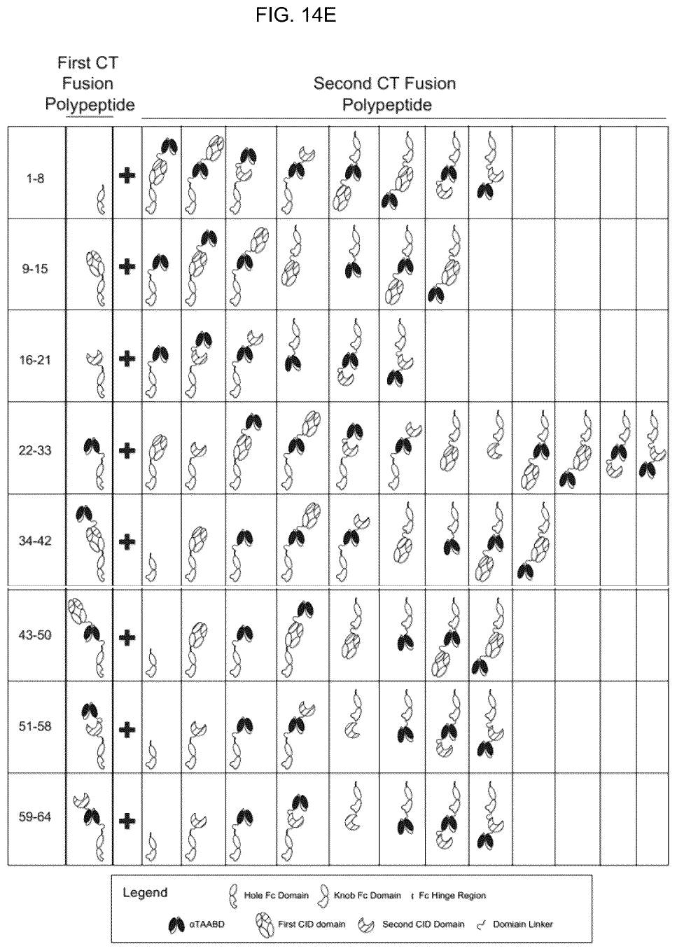

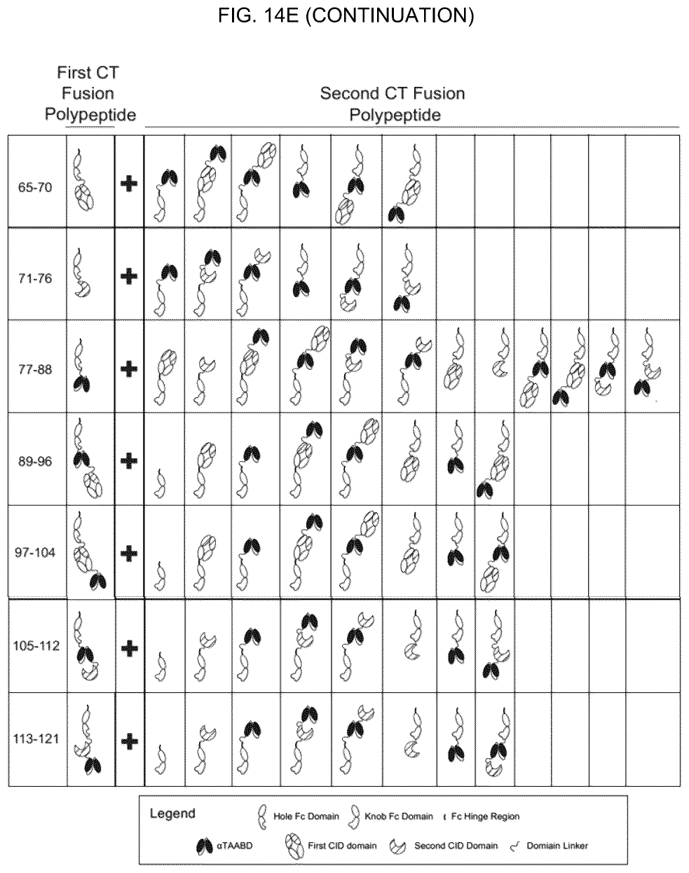

In one aspect, the CT heterodimeric binding proteins comprising a iCID domain and αTTABD comprise two fusion proteins, for example, including, but not limited to, those depicted in FIGS. 14A-14E. The first CT fusion protein comprises one or more iCID domain linked to a first heterodimerization Fc domain via an optional domain linker. The second CT fusion protein comprises an αTTABD linked to a second heterodimerization Fc domain via an optional domain linker. The first and second heterodimerization Fc domains heterodimerize to form the CT heterodimeric binding protein. In some embodiments, the first CT fusion protein comprises a plurality of iCIDs. In some embodiments, the first CT fusion protein comprises two iCIDs, for example, as depicted in FIG. 14C. In additional embodiments, each of the two iCIDs comprises an indinavir binding domain. In some embodiments, the second CT fusion protein comprises a plurality of αTTABD. In some embodiments, the second CT fusion protein comprises two αTTABD, for example, as depicted in FIGS. 14C and 14D.

In some embodiments, the iCID domain is linked to the N terminus of the first heterodimerization Fc domain and the αTTABD is linked to the N terminus of the second heterodimerization Fc domain. In some embodiments, the iCID domain is linked to the N terminus of the first heterodimerization Fc domain and the αTTABD is linked to the C terminus of the second heterodimerization Fc domain. In some embodiments, the iCID domain is linked to the C terminus of the first heterodimerization Fc domain and the αTTABD is linked to the N terminus of the second heterodimerization Fc domain.

The iCID domain and αTTABD can take various formats including a Fab, an scFv, an scFab, and a single domain antibody as described herein above. In some embodiments, both the iCID domain and αTTABD take the format of an scFv. In some embodiments, the iCID domain takes the format of a Fab and the αTTABD take the format of an scFv. In some embodiments, the iCID domain takes the format of an scFab and the αTTABD takes the format of an scFv. In some embodiments, the iCID domain takes the format of a single domain antibody and the αTTABD take the format of an scFv.

In some embodiments, the iCID domain takes the format of a Fab and the αTTABD take the format of n Fab. In some embodiments, the iCID domain takes the format of a scFab and the αTTABD take the format of a Fab. In some embodiments, the iCID domain takes the format of a scFv and the αTTABD take the format of a Fab. In some embodiments, the iCID domain takes the format of a single domain antibody and the αTTABD take the format of a Fab.

In some embodiments, the iCID domain takes the format of a Fab and the αCD3-ABD take the format of an scFab. In some embodiments, the iCID domain takes the format of an scFab and the αTTABD take the format of an scFab. In some embodiments, the iCID domain takes the format of an scFv and the αTTABD take the format of an scFab. In some embodiments, the iCID domain takes the format of a single domain antibody and the αTTABD take the format of an scFab.

In some embodiments, the iCID domain takes the format of an Fab and the αTTABD take the format of a single domain antibody. In some embodiments, the iCID domain takes the format of an scFab and the αTTABD take the format of a single domain antibody. In some embodiments, the iCID domain takes the format of an scFv and the αTTABD take the format of a single domain antibody. In some embodiments, the iCID domain takes the format of a single domain antibody and the αTTABD take the format of a single domain antibody.

In another aspect, the CT heterodimeric binding proteins comprise an iCID domain and two αTTABDs. The two αTTABDs can bind to the same tumor antigen or two different tumor antigens. Advantages of this format can include conferring increased potency to target tumor antigens (TAs) due to increased avidity provided by the two tumor antigen binders. Further, in some embodiments, a TTABD with a lower affinity can be used in this format to increase selectivity of a CT binding protein. Use of multivalent interactions can favor association of a CT binding protein with cells expressing high levels of a TA. Therefore, in some instances, selectivity for high-UA expressing tumor cells can be achieved over healthy tissue expressing lower levels of the TTA. As such, using this format can lower potential side effects of a T-LITE. The first CT fusion protein comprises an iCID domain, an αTTABD, a first heterodimerization Fc domain and optional domain linkers. The second CT fusion protein comprises the other αTTABD linked to a second heterodimerization Fc domain via an optional domain linker. From the N to C terminus, the first CT fusion protein can take various configurations such as αTTABD-optional domain linker-iCID-optional domain linker-Fc, αTTABD-optional domain linker-αTTABD-optional domain linker-iCID-optional domain linker-Fc, iCID-optional domain linker-αTTABD-optional domain linker-Fc, αTTABD-optional domain linker-Fc-optional domain linker-iCID, iCID-optional domain linker-αTTABD-optional domain linker-αTTABD-optional domain linker-Fc, αTTABD-optional domain linker-Fc-optional domain linker-iCID, CID-optional domain linker-Fc-optional domain linker-αTTABD, CID-optional domain linker-Fc-optional domain linker-αTTABD-optional domain linker-αTTABD, Fc-optional domain linker-iCID-optional domain linker-αTTABD, Fc-optional domain linker-iCID-optional domain linker-αTTABD-optional domain linker-αTTABD, Fc-optional domain linker-αTTABD-optional domain linker-iCID, and Fc-optional domain linker-αTTABD-optional domain linker-αTTABD-optional domain linker-iCID. In the second CT fusion protein, the αTTABD can be linked to the N or C terminus of the second heterodimerization Fc domain. The first and second heterodimerization Fc domains heterodimerize to form the CT heterodimeric binding protein. The iCID domain and αTTABD can take various formats including a Fab, an scFv, an scFab, and a single domain antibody as described herein above.

In another aspect, the CT heterodimeric binding proteins comprise a Fc fusion protein and an empty Fc domain. The Fc fusion protein comprises a iCID domain, an αTTABD and a first heterodimerization Fc domain. The empty Fc domain contains a second heterodimerization Fc domain which heterodimerizes with the first heterodimerization Fc domain. The iCID domain and αTTABD can take various formats including a Fab, an scFv, an scFab, and a single domain antibody as described herein above. In some embodiments, the Fc fusion protein may further comprise a second αTTABD linked to the αTTABD via an optional linker. The αTTABD domains in the fusion protein may be identical. From N terminal to C terminal, the Fc fusion protein can have configurations such as iCID-optional domain linker-αTTABD-optional domain linker-Fc, iCID-optional domain linker-αTTABD-optional domain linker-αTTABD-optional domain linker-Fc, αTTABD-optional domain linker-iCID-optional domain linker-Fc, αTTABD-optional domain linker-αTTABD-optional domain linker-iCID-optional domain linker-Fc, αTTABD-optional domain linker-Fc-optional domain linker-iCID, αTTABD-optional domain linker-αTTABD-optional domain linker-Fc-optional domain linker-iCID, iCID-optional domain linker-Fc-optional domain linker-αTTABD, and iCID-optional domain linker-Fc-optional domain linker-αTTABD-optional domain linker-αTTABD. In some embodiments, the Fc fusion protein may further comprise a third iCID domain linked to the second iCID domain via an optional linker. The iCID domains in the fusion protein may be identical.

In one aspect, a composition of the present disclosure comprises a CT heterodimeric binding protein comprising: a) a first CT fusion protein comprising: 1) a second iCID domain; 2) an optional domain linker, and 3) a first heterodimerization Fc domain; and b) a second CT fusion protein comprising: 1) a first anti-tumor targeting ABD (αTTABD); 2) an optional domain linker, and 3) a second heterodimerization Fc domain. In some embodiments, the first CT fusion protein may further comprise a third iCID domain linked to the second iCID domain via an optional linker. The iCID domains may be identical. In some embodiments, the second CT fusion protein may further comprise a second αTTABD linked to the first αTTABD via an optional linker. The αTTABDs may be identical.

In one aspect, a composition of the present disclosure comprises a monomeric CT binding polypeptide comprising: a) a second indinavir chemically induced dimerization (iCID) domain; b) an optional domain linker(s); c) an IgG4 monomeric Fc domain; and d) an anti-tumor targeting ABD (αTTABD). In some embodiments, the monomeric CT binding polypeptide may further comprise a third iCID domain linked to the second iCID domain via an optional linker. The iCID domains may be identical. In some embodiments, the monomeric CT binding polypeptide may further comprise a second αTTABD linked to the first αTTABD via an optional linker. The αTTABDs may be identical.

In some embodiments, the anti-tumor targeting antigen may be EpCAM. In some embodiments, the αTTABD may comprise the composition comprising the EpCAM binding domain described herein.

In some embodiments, the first iCID domain may comprise a composition comprising an indinavir binding domain. In some embodiments, the first iCID domain may comprise a composition comprising an indinavir-complex binding domain. In some embodiments, the composition may comprise one or more heavy chain and/or light chain sequences selected from the group consisting of heavy chain and/or light chain sequences from FIGS. 9C and 9D. In some embodiments, the composition comprises any one of CT heterodimeric binding proteins of FIGS. 9C and 9D. In some embodiments, the composition comprises Ab0386 of FIG. 9C. In some embodiments, the composition comprises Ab0439 of FIG. 9C. In some embodiments, the composition comprises Ab0642 of FIG. 9C. In some embodiments, the composition comprises Ab0643 of FIG. 9C. In some embodiments, the composition comprises Ab0778 of FIG. 9C. In some embodiments, the composition comprises Ab0794 of FIG. 9C. In some embodiments, the composition comprises Ab0795 of FIG. 9C. In some embodiments, the composition comprises Ab0796 of FIG. 9C. In some embodiments, the composition comprises Ab1059 of FIG. 9C. In some embodiments, the composition comprises Ab1068 of FIG. 9C. In some embodiments, the composition comprises Ab1069 of FIG. 9C. In some embodiments, the composition comprises Ab1088 of FIG. 9C. In some embodiments, the composition comprises Ab1089 of FIG. 9C. In some embodiments, the composition comprises Ab1090 of FIG. 9C. In some embodiments, the composition comprises Ab1093 of FIG. 9C. In some embodiments, the composition comprises Ab1094 of FIG. 9C. In some embodiments, the composition comprises Ab1101 of FIG. 9C. In some embodiments, the composition comprises Ab1102 of FIG. 9C. In some embodiments, the composition comprises Ab1111 of FIG. 9C. In some embodiments, the composition comprises Ab1114 of FIG. 9C. In some embodiments, the composition comprises Ab1117 of FIG. 9C. In some embodiments, the composition comprises Ab1121 of FIG. 9C. In some embodiments, the composition comprises Ab1126 of FIG. 9C. In some embodiments, the composition comprises Ab1147 of FIG. 9C. In some embodiments, the composition comprises Ab1148 of FIG. 9C. In some embodiments, the composition comprises Ab1149 of FIG. 9C. In some embodiments, the composition comprises Ab1150 of FIG. 9C. In some embodiments, the composition comprises Ab1151 of FIG. 9C. In some embodiments, the composition comprises Ab1152 of FIG. 9C. In some embodiments, the composition comprises Ab0650 of FIG. 9D. In some embodiments, the composition comprises Ab0651 of FIG. 9D. In some embodiments, the composition comprises Ab0652 of FIG. 9D. In some embodiments, the composition comprises Ab0789 of FIG. 9D. In some embodiments, the composition comprises Ab0790 of FIG. 9D. In some embodiments, the composition comprises Ab1153 of FIG. 9D.

In another aspect, said second iCID domain is an indinavir binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, P5-H5, P2-D8, P3-E12, P6-C8, P7-G8, P6-D7, P6-D5, P3-All, P5-C3, P3-H5, P7-H10, P7-C6, P2-C2, P2-E8, P5-E9, P3-H7, P7-D6, P6-D11, P5-A9, P4-C5, P3-H2, P1-H3, P1-B12, P4-G12, P2-A10, P4-D11, P7-D12, P1-C11, P4-F9, P6-H5, P2-D7, P3-F5, P3-C5, P8-F4, P8-C2, P7-F12, P2-H7, P1-B7, P2-D9, P6-E8, P3-B9, P1-G1, P1-D11, P8-E3, P4-E4, P5-D12, P3-F4, P6-H2, P1-E9, P8-D9, and P4-G11 (FIG. 49). In another aspect, said second iCID domain is an indinavir binding domain comprising a VH domain and VL domain selected from the group consisting of the VH and VL domains of P7-F7, P7-E7, P5-H5, P2-D8, P3-E12, P6-C8, P7-G8, P6-D7, P6-D5, P3-A11, P5-C3, P3-H5, P7-H10, P7-C6, P2-C2, P2-E8, P5-E9, P3-H7, P7-D6, P6-D11, P5-A9, P4-C5, P3-H2, P1-H3, P1-B12, P4-G12, P2-A10, P4-D11, P7-D12, P1-C11, P4-F9, P6-H5, P2-D7, P3-F5, P3-C5, P8-F4, P8-C2, P7-F12, P2-H7, P1-B7, P2-D9, P6-E8, P3-B9, P1-G1, P1-D11, P8-E3, P4-E4, P5-D12, P3-F4, P6-H2, P1-E9, P8-D9, and P4-G11 (FIG. 49). In another aspect, said second iCID is an indinavir-complex binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). In another aspect, said second iCID is an indinavir-complex binding domain comprising a VH domain and VL domain selected from the group consisting of the VH and VL domains of Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50).

### (iv) Useful CT Homodimeric Binding Proteins

In some embodiments, CT binding proteins are homodimeric proteins comprising two identical iCID domains, two identical αTTABDs, optional domain linker(s) and two homodimeric Fc domains.

The iCID domains and αTTABDs can take various formats including an Fab, an scFab, an scFv and a single domain antibody.

In some embodiments, the iCID domains take the format of an Fab comprising a VH-CH1 and VL-CL, and the αTTABDs take the format of an scFv. Accordingly, the CT binding protein comprises two heavy chains containing VH-CH1-hinge domain-Fc domain, and two light chain CT fusion proteins containing, from N to C terminus, VL-CL-domain linker-αTTABD or αTTABD-domain linker-VL-CL.

In some embodiments, the iCID domains take the format of an Fab comprising a VH-CH1 and VL-CL, and the αTTABDs take the format of an scFv. Accordingly, the CT binding protein comprises two heavy chains containing, from N to C terminus, αTTABD-domain linker-VH-CH1-hinge domain-Fc domain, and two light chains containing VL-CL. Alternatively, the CT binding protein comprises two heavy chains containing, from N to C terminus, VH-CH1-hinge domain-Fc domain-domain linker-αTTABD, and two light chains containing VL-CL.

In some embodiments, both the iCID domains and the αTTABDs take the format of an scFv. Accordingly, the CT binding protein comprises two identical fusion proteins of, from N to C terminal, αTTABD-domain linker-CID-optional domain linker-Fc domain, or iCID-domain linker-αTTABD-optional domain linker-Fc domain.

### 8. Exemplary Combinations for Format 1

As described herein, the T-LITE™ compositions are made up of two (or more) different binding proteins, at least one CC binding protein and at least one CT binding protein, which can be combined in various combinations. In the presence of indinavir, the iCID domains of the CC and CT binding proteins form a complex, such that the Format 1 compositions will bind to both CD3 and tumor, becoming active T cell engaging complexes. Any one of the CC binding proteins described herein can be combined with any one of the CT binding proteins described herein.

In one aspect, a T-cell ligand induced transient engager (T-LITE) composition comprises: a) a CC binding protein comprising a first iCID; and b) a CT binding protein comprising a second iCID; wherein one of said first iCID and said second iCID comprises the indinavir binding domain described herein and the other comprises the indinavir-complex binding domain described herein; and wherein in the presence of indinavir said first and second iCID domains form a complex of said first iCID domain-indinavir-said second iCID domain, such that said T-LITE composition will bind both CD3 and said tumor. In some embodiments, a T-cell ligand induced transient engager (T-LITE) composition comprises: a) a CC binding protein comprising two of first iCIDs; and b) two CT binding proteins, each comprising a second iCID; wherein one of said first iCID and said second iCID comprises the indinavir binding domain described herein and the other comprises the indinavir-complex binding domain described herein; and wherein in the presence of indinavir said first and second iCID domains form a complex of said first iCID domain-indinavir-said second iCID domain, such that said T-LITE composition will bind both CD3 and said tumor. In some embodiments, the first iCID comprises the indinavir binding domain described herein, and the second iCID comprises the indinavir-complex binding domain described herein. An exemplary configuration is disclosed as FIG. 1D. In some embodiments, the composition further comprises a second CT binding protein comprising the second iCID domain, wherein the CC binding protein further comprises a third iCID domain that is identical to the first iCID domain.

In one aspect, a T-cell ligand induced transient engager (T-LITE) composition comprises: a) a CC binding protein comprising two of a first iCID; and b) two of a CT binding protein comprising a second iCID; wherein one of said first iCID and said second iCID comprises the indinavir binding domain described herein and the other comprises the indinavir-complex binding domain described herein; and wherein in the presence of indinavir said first and second iCID domains form a complex of said first iCID domain-indinavir-said second iCID domain, such that said T-LITE composition will bind both CD3 and said tumor. In some embodiments, a T-cell ligand induced transient engager (T-LITE) composition comprises: a) a CC binding protein comprising two of first iCIDs; and b) two CT binding proteins, each comprising a second iCID; wherein one of said first iCID and said second iCID comprises the indinavir binding domain described herein and the other comprises the indinavir-complex binding domain described herein; and wherein in the presence of indinavir said first and second iCID domains form a complex of said first iCID domain-indinavir-said second iCID domain, such that said T-LITE composition will bind both CD3 and said tumor. In some embodiments, the first iCID comprises the indinavir binding domain described herein, and the second iCID comprises the indinavir-complex binding domain described herein. An exemplary configuration is disclosed as FIG. 1D.

### 9. Format 2 Complexes

As generally outlined herein, the invention provides pairs of binding proteins (e.g. a CC binding protein and a CTCoS binding protein) that together, in the presence of indinavir, to form a T cell engaging complex. Generally, each binding protein is in turn made up of either two fusion proteins (that together form either a CC binding protein or a CTCoS binding protein), or a monomeric fusion polypeptide as outlined below. An exemplary configuration for Format 2 is depicted in FIG. 2. As will be appreciated by those in the art, the CC binding proteins and the CTCoS binding proteins can each be independently selected from monomeric fusion polypeptides, homodimeric fusion proteins and heterodimeric fusion proteins. As described herein, the T-LITE™ compositions are made up of two (or more) different binding proteins, at least one CC binding protein and at least one CT binding protein, which can be combined in various combinations. In the presence of indinavir, the iCID domains of the CC and CT binding proteins form a complex, such that the Format 1 compositions will bind to both CD3 and tumor, becoming active T cell engaging complexes.

Any one of the CC binding proteins described herein can be combined with any one of the CTCoS binding proteins described herein.

In one aspect, a composition of the present disclosure comprises a CTCoS heterodimeric binding protein comprising: a) a first CTCoS fusion protein comprising: i) a first iCID domain; ii) optional domain linker(s); and iii) a first heterodimerization Fc domain; and b) a second CTCoS fusion protein comprising: i) the anti-tumor targeting antigen binding domain (αTTABD) described herein; ii) optional domain linker(s); and iii) a second heterodimerization Fc domain; wherein one of said first and second CTCoS fusion proteins further comprises a co-stimulatory domain.

In some embodiments, the anti-tumor targeting antigen may be EpCAM. In some embodiments, the αTTABD may comprise the composition comprising the EpCAM binding domain described herein.

In some embodiments, the first iCID domain may comprise a composition comprising an indinavir binding domain. In some embodiments, the first iCID domain may comprise a composition comprising an indinavir-complex binding domain.

In some embodiments, the composition further comprises a second CTCos binding protein comprising the second iCID domain, wherein the CC binding protein further comprises a third iCID domain that is identical to the first iCID domain.

In one aspect, a co-stimulatory T-cell ligand induced transient engager (BrighT-LITE) composition comprises: a) a CC binding protein and b) a CTCoS binding protein; wherein in the presence of indinavir said first and second iCID domains form a complex of said first iCID domain-said indinavir-said second iCID domain, such that said BrighT-LITE composition binds both CD3 and said tumor, wherein one of said first iCID and said second iCID comprises the indinavir binding domain described herein and the other comprises the indinavir-complex binding domain described herein, and wherein in the presence of indinavir said first and second iCID domains form a complex of said first iCID domain-indinavir-said second iCID domain, such that said T-LITE composition will bind both CD3 and said tumor. In some embodiments, the composition further comprises a second CTCoS binding protein comprising the second iCID domain, wherein the CC binding protein further comprises a third iCID domain that is identical to the first iCID domain.

a. CC Binding Proteins

The CC binding proteins of Format 2 may be the same as those for Format 1 (and Format 3). Thus, the invention provides CC fusion polypeptides that form the CC binding protein(s) of the invention. Each CC binding protein contains a first CID domain, and an anti-CD3 ABD. In some embodiments, the CC binding protein does not contain an Fc domain, such as direct fusion of a first iCID domain and an αCD3-ABD. Both the iCID domain and the αCD3-ABD can take the format of an scFv, a Fab, an scFab or a single domain antibody such as the VHH of camelid derived single domain antibody. In some embodiments, the CC binding protein contains an Fc domain. In some cases, the CC binding protein is monomeric. It can include an iCID domain directly fused to an αCD3-ABD, or it can also rely on the use of a monomeric Fc domain, as more fully outlined below. In some embodiments, the CC binding protein comprises a first and a second CC fusion polypeptide, that come together as dimers, either heterodimeric or homodimeric, to provide the αCD3-ABD functionally coupled to an iCID domain. In some embodiments, the iCID domain may be linked with another iCID domain via an optional domain linker as described above.

### (i) Monomeric CC Fusion Polypeptides

In some embodiments, the CC binding protein is monomeric and relies on the use of a monomeric IgG4 Fc domain. In some embodiments, the CC binding proteins are monomeric proteins comprising an iCID domain, an αCD3-ABD, optional domain linker(s) and an IgG4 monomeric Fc domain. The CC binding polypeptide can be a fusion polypeptide with a structure selected from the group, from N- to C-terminal: iCID domain-optional domain linker-αCD3-ABD-optional domain linker-Fc domain; αCD3-ABD-optional domain linker-iCID domain-optional domain linker-Fc domain; iCID domain-optional domain linker-Fc domain-optional domain linker-αCD3-ABD; αCD3-ABD-optional domain linker-Fc domain-optional domain linker-iCID domain; Fc domain-optional domain linker-αCD3-ABD-optional domain linker-iCID domain; Fc domain-optional domain linker-iCID domain-optional domain linker-αCD3-ABD; and iCID domain-optional domain linker-iCID domain-optional domain linker-αCD3 ABD-optional domain linker-Fc domain. Either or both of the CID and αCD3-ABD can take any one of the formats including Fab, scFv, scFab, a single domain antibody such as the VHH of camelid derived single domain antibody.

In some instances, the selected arrangements of domains of the monomeric CC binding protein employed in the BrighT-LITE compositions provide an improvement, e.g., in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

### (ii) Dimeric CC Binding Proteins

In some embodiments, the CC binding protein comprises a first and a second CC fusion polypeptide, that come together as dimers, either heterodimeric or homodimeric, to provide the αCD3-ABD functionally coupled to an iCID domain. In these embodiments, the CC binding proteins rely on the use of Fc domains that are dimers, either heterodimeric Fc domains or homodimeric Fc domains. In some embodiments, the CC binding proteins are CC heterodimeric binding proteins that use heterodimerization variants in the Fc domains. Thus, in some embodiments, the CC binding protein comprises a first and a second CC fusion polypeptide, wherein one of the first and second CC fusion polypeptides contains the αCD3-ABD and the other the iCID domain. In some embodiments, the CC binding protein comprises a first CC fusion polypeptide which contains both the αCD3-ABD and the iCID domain, and a second CC fusion polypeptide comprising an empty Fc domain. In these embodiments, the first and second CC fusion polypeptides can have the structures (from N- to C-terminal, with “DL” standing for “domain linker”) shown in Table 4. In some embodiments, each of the CC fusion polypeptide having one iCIDs may further comprise another iCID linked to the iCID via an optional DL, for example, as shown in 37-40 in Table 4. The iCIDs in the same fusion polypeptide may be identical. In some embodiments, the iCIDs in the same fusion polypeptide may be different. The DLs in the same fusion polypeptide may be different. In some embodiments, the DLs in the same fusion polypeptide may be same.

As discussed herein, each of the iCID domains and αCD3-ABD domains of Table 4 can be selected from a Fab, an scFab, an scFvs or a single domain antibody such as the VHH of camelid derived single domain antibody. The Fc domains in the first CC fusion polypeptide and second CC fusion polypeptide heterodimerize with each other. The iCID domain(s) in the first CC fusion polypeptide and/or second CC fusion polypeptide can be selected from either half of the iCID domain pairs described herein.

In some embodiments, the CC binding proteins are CC homodimeric binding proteins that use standard Fc domains that self-assemble to form homodimers. In some embodiments, one of either of the iCID domain or the αCD3-ABD is formed using the VH and VL of a traditional, tetrameric antibody, and the other is attached to either the N- or C-terminus of the light chain or the N-terminus of the heavy chain. In some embodiments, one of either of the iCID domain or the αCD3-ABD is formed using the VH and VL of a traditional, tetrameric antibody, and the other is attached to C-terminus of Fc domain. For example, the iCID domain can take a Fab format, and the αCD3-ABD can take an scFv format attached to the C-terminus of the Fc domain. Alternatively, the αCD3-ABD can take a Fab format, and the iCID domain can take an scFv format attached to the C-terminus of the Fc domain. In some embodiments, both the iCID domain and the αCD3-ABD take the format of an scFv or scFab. From N- to C-terminal, the CC binding protein comprises iCID domain-optional domain linker-αCD3-ABD-optional domain linker-homodimeric Fc domain or αCD3-ABD-optional domain linker-iCID domain-optional domain linker-homodimeric Fc domain.

In some instances, the selected arrangements of domains of the dimeric CC binding protein employed in the BrighT-LITE composition provide for an improvement, e.g. in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

(iii) Useful CC Heterodimeric Binding Proteins

As discussed herein, useful CC heterodimeric binding proteins are generally shown in FIGS. 2, 12A-12G and 13A-13C.

The iCID domain and αCD3-ABD can take various formats including an Fab, an scFv, an scFab, and a single domain antibody as described herein above. In some embodiments, both the iCID domain and αCD3 ABD take the format of an scFv. In some embodiments, the iCID domain takes the format of an Fab and the αCD3 ABD take the format of an scFv. In some embodiments, the iCID domain takes the format of an scFab and the αCD3 ABD take the format of an scFv. In some embodiments, the iCID domain takes the format of a single domain antibody and the αCD3-ABD take the format of an scFv.

In some embodiments, the iCID domain takes the format of a Fab and the αCD3 ABD take the format of an Fab. In some embodiments, the iCID domain takes the format of an scFab and the αCD3 ABD take the format of an Fab. In some embodiments, the iCID domain takes the format of an scFv and the αCD3 ABD take the format of an Fab. In some embodiments, the iCID domain takes the format of a single domain antibody and the αCD3-ABD take the format of a Fab.

In some embodiments, the iCID domain takes the format of an Fab and the αCD3 ABD take the format of an scFab. In some embodiments, the iCID domain takes the format of an scFab and the αCD3 ABD take the format of an scFab. In some embodiments, the iCID domain takes the format of an scFv and the αCD3 ABD take the format of an scFab. In some embodiments, the iCID domain takes the format of a single domain antibody and the αCD3 ABD take the format of an scFab.

In some embodiments, the iCID domain takes the format of an Fab and the αCD3-ABD take the format of a single domain antibody. In some embodiments, the iCID domain takes the format of an scFab and the αCD3 ABD take the format of a single domain antibody. In some embodiments, the iCID domain takes the format of an scFv and the αCD3 ABD take the format of a single domain antibody. In some embodiments, the iCID domain takes the format of a single domain antibody and the αCD3 ABD take the format of a single domain antibody.

In another aspect, the CC heterodimeric binding proteins comprises a Fc fusion protein and an empty Fc domain. The Fc fusion protein comprises a iCID domain, an αCD3 ABD, a first heterodimerization Fc domain and one or more optional linkers. The empty Fc domain contains a second heterodimerization Fc domain which heterodimerizes with the first heterodimerization Fc domain.

The iCID domain and αCD3 ABD can take various formats including an Fab, an scFv, an scFab, or a single domain antibody as described herein above. In some embodiments, the iCID takes the Fab format, and the αCD3 ABD takes the format of an Fab, an scFv, an scFab, or a single domain antibody. In some embodiments, the iCID takes the scFab format, and the αCD3 ABD takes the format of an Fab, an scFv, an scFab, or a single domain antibody. In some embodiments, the iCID takes the scFv format, and the αCD3 ABD takes the format of an Fab, an scFv, an scFab, or a single domain antibody. In some embodiments, the iCID takes the single domain antibody format, and the αCD3 ABD takes the format of an Fab, an scFv, an scFab, or a single domain antibody.

From N to C terminus, the Fc fusion protein can have configurations, such as iCID domain-optional domain linker-αCD3 ABD-optional domain linker-Fc, first iCID domain-optional domain linker-second iCID domain-optional domain linker-αCD3 ABD-optional domain linker-Fc, αCD3 ABD-optional domain linker-iCID domain-optional domain linker-Fc, αCD3 ABD-optional domain linker-first iCID domain-optional domain linker-second iCID domain-optional domain linker-Fc, αCD3 ABD-optional domain linker-Fc-optional domain linker-iCID domain, αCD3 ABD-optional domain linker-Fc-optional domain linker-first iCID domain-optional domain linker-second iCID domain, iCID domain-optional domain linker-Fc-optional domain linker-αCD3 ABD, and first iCID domain-optional domain linker-second iCID domain-optional domain linker-Fc-optional domain linker-αCD3 ABD. Further, from N to C terminus, the Fc fusion protein can have configurations, such as Fc-linker-iCID domain-optional domain linker-CD3 ABD, Fc-optional domain linker-CD3 ABD-optional domain linker-iCID domain, iCID domain-optional domain linker-Fc-liker-CD3 ABD, Fc-optional domain linker-first iCID domain-optional linker-second iCID domain-optional linker-CD3 ABD, Fc-Linker-CD3 ABD-optional domain linker-first iCID domain-optional domain linker-second iCID domain, and first iCID domain-optional domain linker-second iCID domain-optional domain linker-Fc-liker-CD3 ABD. Additionally, from N to C terminus, the Fc fusion protein can have configurations, such as iCID domain-optional domain linker-Fc, Fc-optional domain linker-iCID domain, CD3 ABD-optional domain linker-Fc, Fc-optional domain linker-CD3 ABD, first iCID domain-optional domain linker-Fc-optional domain linker-second iCID domain, first iCID domain-optional domain linker-second iCID domain-optional domain linker-Fc, Fc-optional domain linker-first iCID domain-optional domain linker-second iCID domain, CD3 ABD-optional domain linker-Fc, Fc-optional domain linker-CD3 ABD, first iCID domain-optional domain linker-second iCID domain-optional domain linker-Fc, and Fc-optional domain linker first iCID domain-optional domain linker-second iCID domain. The first and second iCID domains may be identical.

In one aspect, a composition described herein comprises a CC heterodimeric binding protein comprising; i) a first CC fusion protein comprising: 1) a first indinavir chemically induced dimerization (iCID) domain; 2) an optional linker; and 3) a first heterodimerization Fc domain; and ii) a second CC fusion protein comprising: 1) an anti-CD3 antigen binding domain (ABD; αCD3-ABD); 2) an optional domain linker, and 3) a second heterodimerization Fc domain. In some embodiments, the first CC fusion protein may further comprise a second iCID domain linked to the first iCID domain via an optional linker. The first and second iCID domains may be identical.

b. CTCoS Binding Proteins

Similar to CC binding proteins, the invention provides CTCoS binding proteins. Each CTCoS binding protein comprises an iCID domain, an anti-TTABD (αTTABD), and one or more co-stimulatory domains. In some embodiments, CTCoS binding proteins do not contain an Fc domain, such as direct fusion of an iCID domain, an αTTABD and one or more co-stimulatory domains. In some embodiments, CTCoS binding proteins contain an Fc domain. In some embodiments, CTCoS binding proteins comprise a first and a second CTCoS fusion polypeptide, that come together as dimers, either heterodimerically or homodimerically, to provide functional coupling of an αTTABD, one or more co-stimulatory domains and an iCID domain. Any of the iCID domain, the αTTABD and the co-stimulatory domain(s) can take the format of an scFv, a Fab, an scFab or a single domain antibody such as the VHH of camelid derived single domain antibody.

### (i) Dimeric CTCoS Binding Proteins

In some embodiments, the CTCoS binding protein comprises a first and a second CTCoS fusion polypeptide, that come together heterodimerically to provide the functional coupling of an αTTABD, one or more co-stimulatory domains and an iCID domain. In these embodiments, the CTCoS binding proteins rely on the use of heterodimerization variants in the Fc domains. Thus, in some embodiments, the CTCoS binding protein comprises a first and a second CTCoS fusion protein, wherein one of the first and second CTCoS fusion protein contains the αTTABD and the other the iCID domain, and wherein either or both the first and second CTCoS fusion proteins also contains one or more of the co-stimulatory domains.

Accordingly, in some embodiments, the first CTCoS fusion protein comprises an iCID domain, and the second CTCoS fusion protein comprises an αTTABD and a co-stimulatory domain. In some embodiments, the first CTCoS fusion protein comprises an iCID domain and a co-stimulatory domain, and the second CTCoS fusion protein comprises an αTTABD. In some embodiments, the first CTCoS fusion protein comprises an iCID domain and a first co-stimulatory domain, and the second CTCoS fusion protein comprises an αTTABD and a second co-stimulatory domain. In some embodiments, the first CTCoS fusion protein comprises an iCID domain, and the second CTCoS fusion protein comprises an αTTABD, a first co-stimulatory domain and a second co-stimulatory domain. In some embodiments, the first CTCoS fusion protein comprises an iCID domain, a first co-stimulatory domain and a second co-stimulatory domain; and the second CTCoS fusion protein comprises an (TABD. In some instances, the first and second co-stimulatory domains are identical. In some instances, the first and second co-stimulatory domains are different molecules.

Table 6 provides exemplary formats of the first and second CTCoS fusion protein that can be coupled to form a BrighT-LITE in the presence of an iCID small molecule (“DL” standing for “optional domain linker” and “CoS” standing for “co-stimulatory domain”). In some embodiments, the CTCoS fusion protein comprising an iCID domain may further comprise another iCID domain linked to the iCID domain via an optional domain linker as described above for Format 1 above. In some embodiments, the CTCoS fusion protein comprising a αTTABD may further comprise another αTTABD linked to the αTTABD via an optional domain linker as described above for Format 1 above.

TABLE 6

| | First CTCoS fusion protein | Second CTCoS fusion protein |
|---|---|---|
| | (N- to C- terminal) | (N- to C- terminal) |
| 1 | iCID-DL-Fc domain | Cos-DL-αTTABD-DL-Fc domain |
| 2 | iCID-DL-Fc domain | αTTABD-DL-CoS-DL-Fc domain |
| 3 | iCID-DL-Fc domain | Fc domain-DL-αTTABD-DL-CoS |
| 4 | iCID-DL-Fc domain | Fc domain-DL-CoS-DL-αTTABD-DL |
| 5 | αTTABD-DL-Fc domain | CoS-DL-iCID-DL-Fc domain |
| 6 | αTTABD-DL-Fc domain | iCID-DL-CoS-DL-Fc domain |
| 7 | αTTABD-DL-Fc domain | Fc domain-DL-CoS-DL-iCID |
| 8 | αTTABD-DL-Fc domain | Fc domain-DL-iCID-DL-CoS-DL |
| 9 | αTTABD-DL-iCID-DL-Fc domain | CoS-DL-Fc domain |
| 10 | αTTABD-DL-iCID-DL-Fc domain | Fc domain-DL-CoS |
| 11 | αTTABD-DL-iCID-DL-Fc domain | CoS-DL-CID-DL-Fc domain |
| 12 | αTTABD-DL-iCID-DL-Fc domain | iCID-DL-CoS-DL-Fc domain |
| 13 | αTTABD-DL-iCID-DL-Fc domain | Fc domain-DL-CoS-DL-iCID |
| 14 | αTTABD-DL-iCID-DL-Fc domain | Fc domain-DL-iCID-DL-CoS-DL |
| 15 | iCID-DL-αTTABD-DL-Fc domain | CoS-DL-Fc domain |
| 16 | iCID-DL-αTTABD-DL-Fc domain | Fc domain-DL-CoS |
| 17 | iCID-DL-αTTABD-DL-Fc domain | CoS-DL-iCID-DL-Fc domain |
| 18 | iCID-DL-αTTABD-DL-Fc domain | iCID-DL-CoS-DL-Fc domain |
| 19 | iCID-DL-αTTABD-DL-Fc domain | Fc domain-DL-CoS-DL-iCID |
| 20 | iCID-DL-αTTABD-DL-Fc domain | Fc domain-DL-iCID-DL-CoS-DL |
| 21 | Fc domain-DL-iCID | Cos-DL-αTTABD-DL-Fc domain |
| 22 | Fc domain-DL-iCID | αTTABD-DL-CoS-DL-Fc domain |
| 23 | Fc domain-DL-iCID | Fc domain-DL-αTTABD-DL-CoS |
| 24 | Fc domain-DL-iCID | Fc domain-DL-CoS-DL-αTTABD-DL |
| 25 | Fc domain-DL-αTTABD | CoS-DL-Fc domain |
| 26 | Fc domain-DL-αTTABD | Fc domain-DL-CoS |
| 27 | Fc domain-DL-αTTABD | CoS-DL-iCID-DL-Fc domain |
| 28 | Fc domain-DL-αTTABD | iCID-DL-CoS-DL-Fc domain |
| 29 | Fc domain-DL-αTTABD | Fc domain-DL-CoS-DL-iCID |
| 30 | Fc domain-DL-αTTABD | Fc domain-DL-iCID-DL-CoS-DL |
| 31 | Fc domain-DL-αTTABD-DL-iCID | CoS-DL-Fc domain |
| 32 | Fc domain-DL-αTTABD-DL-iCID | Fc domain-DL-CoS |
| 33 | Fc domain-DL-αTTABD-DL-iCID | CoS-DL-iCID-DL-Fc domain |
| 34 | Fc domain-DL-αTTABD-DL-iCID | iCID-DL-CoS-DL-Fc domain |
| 35 | Fc domain-DL-αTTABD-DL-iCID | Fc domain-DL-CoS-DL-iCID |
| 36 | Fc domain-DL-αTTABD-DL-iCID | Fc domain-DL-iCID-DL-CoS-DL |
| 37 | Fc domain-DL-iCID-DL-αTTABD-DL | CoS-DL-Fc domain |
| 38 | Fc domain-DL-iCID-DL-αTTABD-DL | Fc domain-DL-CoS |
| 39 | Fc domain-DL-iCID-DL-αTTABD-DL | CoS-DL-CID-DL-Fc domain |
| 40 | Fc domain-DL-iCID-DL-αTTABD-DL | iCID-DL-CoS-DL-Fc domain |
| 41 | Fc domain-DL-iCID-DL-αTTABD-DL | Fc domain-DL-CoS-DL-iCID |
| 42 | Fc domain-DL-iCID-DL-αTTABD-DL | Fc domain-DL-iCID-DL-CoS-DL |

As discussed herein, each of the iCID domains, αTTABD, and co-stimulatory domains of Table 6 can be selected from a Fab, an scFab, an scFvs or a single domain antibody such as the VHH of camelid derived single domain antibody. The Fc domains in the first CTCoS fusion protein and second CTCoS fusion protein heterodimerize with each other. The iCID domain in the first CTCoS fusion protein can be selected from one half of the iCID domain pairs described herein. The iCID domain in the second CTCoS fusion protein can be selected from the other half of the iCID domain pairs.

In some instances, the selected arrangements of domains of the dimeric CTCoS binding proteins employed in the BrighT-LITE compositions provide an improvement, e.g. in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-old, 8-fold, 9-fold, 10-fold or higher increase in, e.g. stability, affinity, or effector activity, is observed.

### (ii) Useful CTCoS Heterodimeric Binding Proteins

In one aspect, the CTCoS heterodimeric binding proteins comprising an iCID domain, an αTTABD, and a co-stimulatory domain comprise a first CTCoS fusion protein and a second CTCoS fusion protein. The first CTCoS fusion protein comprises an iCID domain linked to a first heterodimerization Fc domain via an optional domain linker. The second CTCoS fusion protein comprises an αTTABD linked to a second heterodimerization Fc domain via an optional domain linker. The co-stimulatory domain is either included in the first CTCoS fusion protein or the second CTCoS fusion protein. The first and the second heterodimerization Fc domains heterodimerize to form the CTCoS heterodimeric binding protein.

In some embodiments, the first CTCoS fusion protein comprises an iCID domain and a first heterodimerization Fc domain, and the second CTCoS fusion protein comprises an αTTABD, a co-stimulatory domain and a second heterodimerization Fc domain. The iCID domain can be linked to the N terminus or the C terminus of the first heterodimerization Fc domain in the first CTCoS fusion protein. In the second CTCoS fusion protein, the αTTABD or the co-stimulatory domain can be linked to the N terminus or the C terminus of the second heterodimerization Fc domain. In some embodiments, both the αTTABD and the co-stimulatory domain are linked to the N terminus of the second heterodimerization Fc domain, e.g., from the N to the C terminal, in the format of αTTABD-CoS-Fc domain or CoS-αTTABD-Fc domain. In some embodiments, the αTTABD is linked to the N terminus of the second heterodimerization Fc domain, and the co-stimulatory domain is linked to the C terminus of the second heterodimerization Fc domain. In some embodiments, the co-stimulatory domain is linked to the N terminus of the second heterodimerization Fc domain, and the αTTABD is linked to the C terminus of the second heterodimerization Fc domain. In some embodiments, the composition further comprises a second CTCos binding protein comprising the second iCID domain, wherein the CC binding protein further comprises a third iCID domain that is identical to the first iCID domain.

The iCID domain and the αTTABD can take various formats including a Fab, an scFv, an scFab, and a single domain antibody as described herein. The co-stimulatory domain can be antibody fragment or a ligand as described herein. When the co-stimulatory domain is an antibody fragment, it can take various formats including a Fab, an scFv, an scFab, and a single domain antibody as described herein. For example, in some embodiments, the iCID domain, αTTABD, and co-stimulatory domain take the format of an scFv. In some embodiments, the iCID domain takes the format of a Fab and the αTTABD and co-stimulatory domain take the format of an scFv. In some embodiments, the iCID domain takes the format of an scFab and the αTTABD and co-stimulatory domain take the format of an scFv. In some embodiments, the iCID domain takes the format of a Fab, an scFv, an scFab, or a single domain antibody; the αTTABD takes the format of a Fab composed of VH-CH1 and VL-CL; and the co-stimulatory domain take the format of an scFv. The αTTABD is linked to the second heterodimerization Fc domain via an optional domain linker, and the co-stimulatory domain is linked to the N terminus of VH-CH1, the N or C terminus of VL-CL, or the C terminus of the second heterodimerization Fc domain. In some embodiments, the iCID domain takes the format of a Fab, an scFv, an scFab, or a single domain antibody; the co-stimulatory domain takes the format of a Fab composed of VH-CH1 and VL-CL; and the αTTABD take the format of an scFv. The co-stimulatory domain is linked to the second heterodimerization Fc domain via an optional domain linker, and the αTTABD is linked to the N terminus of VH-CH1, the N or C terminus of VL-CL, or the C terminus of the second heterodimerization Fc domain. In some embodiments, the iCID domain takes the format of a Fab, an scFv, an scFab, or a single domain antibody linked to the first heterodimerization Fc domain. The αTTABD takes the format of a Fab, an scFv, an scFab, or a single domain antibody and is linked to the second heterodimerization Fc domain. The co-stimulatory domain is a ligand, and is linked to either the N terminus of the αTTABD or the C terminus of the second heterodimerization Fc domain.

In some embodiments, the first CTCoS fusion protein comprises an iCID domain, a co-stimulatory domain and a first heterodimerization Fc domain; and the second CTCoS fusion protein comprises an αTTABD and a second heterodimerization Fc domain. The αTTABD can be linked to the N terminus or the C terminus of the second heterodimerization Fc domain in the second CTCoS fusion protein. In the first CTCoS fusion protein, the iCID domain or the co-stimulatory domain can be linked to the N terminus or the C terminus of the first heterodimerization Fc domain. In some embodiments, both the iCID domain and the co-stimulatory domain are linked to the N terminus of the first heterodimerization Fc domain, e.g., from the N to the C terminal, in the format of iCID-CoS-Fc domain or CoS-iCID-Fc domain. In some embodiments, the iCID domain is linked to the N terminus of the first heterodimerization Fc domain, and the co-stimulatory domain is linked to the C terminus of the first heterodimerization Fc domain. In some embodiments, the co-stimulatory domain is linked to the N terminus of the first heterodimerization Fc domain, and the iCID domain is linked to the C terminus of the first heterodimerization Fc domain.

The iCID domain, and αTTABD can take various formats including a Fab, an scFv, an scFab, and a single domain antibody as described herein. The co-stimulatory domain can be antibody fragment or a ligand as described herein. When the co-stimulatory domain is an antibody fragment, it can take various formats including a Fab, an scFv, an scFab, and a single domain antibody as described herein. For example, in some embodiments, both the iCID domain, αTTABD, and co-stimulatory domain take the format of an scFv. In some embodiments, the αTTABD takes the format of a Fab and the iCID domain and co-stimulatory domain take the format of an scFv. In some embodiments, the αTTABD takes the format of an scFab, and the iCID domain and co-stimulatory domain take the format of an scFv. In some embodiments, the αTTABD takes the format of a Fab, an scFv, an scFab, or a single domain antibody; the iCID domain takes the format of a Fab composed of VH-CH1 and VL-CL; and the co-stimulatory domain take the format of an scFv. The αTTABD is linked to the first heterodimerization Fc domain via an optional domain linker, and the co-stimulatory domain is linked to the N terminus of VH-CH1 of the iCID domain, the N or C terminus of VL-CL of the iCID domain, or the C terminus of the first heterodimerization Fc domain. In some embodiments, the αTTABD takes the format of a Fab, an scFv, an scFab, or a single domain antibody; the co-stimulatory domain takes the format of a Fab composed of VH-CH1 and VL-CL; and the iCID domain take the format of an scFv. The co-stimulatory domain is linked to the first heterodimerization Fc domain via an optional domain linker, and the iCID domain is linked to the N terminus of VH-CH1 of the αTTABD, the N or C terminus of VL-CL of the αTTABD, or the C terminus of the first heterodimerization Fc domain. In some embodiments, the αTTABD takes the format of a Fab, an scFv, an scFab, or a single domain antibody linked to the second heterodimerization Fc domain. The iCID takes the format of a Fab, an scFv, an scFab, or a single domain antibody and is linked to the first heterodimerization Fc domain. The co-stimulatory domain is a ligand, and is linked to either the N terminus of the iCID domain or the C terminus of the first heterodimerization Fc domain.

In another aspect, the CTCoS heterodimeric binding proteins comprising an iCID domain, an αTTABD, and two co-stimulatory domain comprise a first CTCoS fusion protein and a second CTCoS fusion protein. In some embodiments, the first CTCoS fusion protein comprises an iCID domain, a first co-stimulatory domain, and a first heterodimerization Fc domain. The second CTCoS fusion protein comprises an αTTABD, a second co-stimulatory domain, and a second heterodimerization Fc domain. In some embodiments, the first CTCoS fusion protein comprises an iCID domain, and a first heterodimerization Fc domain. The second CTCoS fusion protein comprises an αTTABD, a first and a second co-stimulatory domains, and a second heterodimerization Fc domain. In some embodiments, the first CTCoS fusion protein comprises an iCID domain, a first and a second co-stimulatory domains, and a first heterodimerization Fc domain. The second CTCoS fusion protein comprises an αTTABD and a second heterodimerization Fc domain. In some instances, the first and second co-stimulatory domains are identical. In some instances, the first and second co-stimulatory domains are different molecules.

In some embodiments, the first CTCoS fusion protein comprises an iCID domain, a first co-stimulatory domain (referred to as “CoS1”), and a first heterodimerization Fc domain. Exemplary formats include, from the N terminal to C terminal, iCID-DL-CoS1-DL-Fc domain, CoS1-DL-iCID-DL-domain, CoS1-DL-Fc domain-DL-iCID, iCID-DL-Fc domain-DL-CoS1, Fc domain-DL-CoS1-DL-iCID, or Fc domain-DL-iCID-DL-CoS1. The second CTCoS fusion protein comprises an αTTABD, a second co-stimulatory domain (referred to as “CoS2”), and a second heterodimerization Fc domain. Optional domain linkers (referred to as “DL”) are used to link the various domains. Exemplary formats include, from the N terminal to C terminal, αTTABD-DL-CoS2-DL-Fc domain, CoS2-DL-αTTABD-DL-Fc domain, CoS2-DL-Fc domain-DL-αTTABD, αTTABD-DL-Fc domain-DL-CoS2, Fc domain-DL-CoS2-DL-αTTABD, or Fc domain-DL-αTTABD-DL-CoS2.

In some embodiments, the first CTCoS fusion protein comprises an iCID domain and a first heterodimerization Fc domain. The second CTCoS fusion protein comprises an αTTABD, a first co-stimulatory domain (referred to as CoS1), a second co-stimulatory domain (referred to as CoS2), and a second heterodimerization Fc domain. Exemplary formats include, from the N terminal to C terminal, αTTABD-DL-CoS1-DL-CoS2-DL-Fc domain, CoS1-DL-CoS2-DL-αTTABD-DL-Fc domain, CoS1-DL-αTTABD-DL-CoS2-DL-Fc domain, CoS1-DL-Fc domain-DL-αTTABD-DL-CoS2, CoS1-DL-Fc domain-DL-CoS2-DL-αTTABD, CoS1-DL-αTTABD-DL-Fc domain-DL-CoS2, αTTABD-DL-CoS1-DL-Fc domain-DL-CoS2, αTTABD-DL-Fc domain-DL-CoS1-DL-CoS2, Fc domain-DL-CoS1-DL-αTTABD-DL-CoS2, Fc domain-DL-αTTABD-DL-CoS1-DL-CoS2, and Fc domain-DL-CoS1-DL-CoS2-DL-αTTABD. In these embodiments, CoS1 and CoS2 can exchange positions in the second CTCoS fusion proteins.

In some embodiments, the first CTCoS fusion protein comprises an iCID domain, a first co-stimulatory domain (referred to as CoS1), a second co-stimulatory domain (referred to as CoS2), and a first heterodimerization Fc domain. The second CTCoS fusion protein comprises an αTTABD and a second heterodimerization Fc domain. Exemplary formats include, from the N terminal to C terminal, iCID-DL-CoS1-DL-CoS2-DL-Fc domain, CoS1-DL-CoS2-DL-iCID-DL-Fc domain, CoS1-DL-iCID-DL-CoS2-DL-Fc domain, CoS1-DL-Fc domain-DL-iCID-DL-CoS2, CoS1-DL-Fc domain-DL-CoS2-DL-iCID, CoS1-DL-iCID-DL-Fc domain-DL-CoS2, iCID-DL-CoS1-DL-Fc domain-DL-CoS2, iCID-DL-Fc domain-DL-CoS1-DL-CoS2, Fc domain-DL-CoS1-DL-iCID-DL-CoS2, Fc domain-DL-iCID-DL-CoS1-DL-CoS2, and Fc domain-DL-CoS1-DL-CoS2-DL-iCID. In these embodiments, CoS1 and CoS2 can exchange positions in the first CTCoS fusion proteins.

The iCID domain, and αTTABD can take various formats including a Fab, an scFv, an scFab, and a single domain antibody as described herein. Either the first or the second co-stimulatory domains can be an antibody fragment or a ligand as described herein. In some embodiments, the first co-stimulatory domains is an antibody fragment, and the second co-stimulatory domain is a ligand. In some embodiments, the first co-stimulatory domains is a ligand, and the second co-stimulatory domain is an antibody fragment. In some embodiments, the first and second co-stimulatory domains are ligands. In some embodiments, the first and second co-stimulatory domains are antibody fragments. When the co-stimulatory domains are antibody fragments, they can take various formats including a Fab, an scFv, an scFab, and a single domain antibody as described herein.

For example, in some embodiments, the first CTCoS fusion protein comprises an iCID domain linked to a first heterodimerization Fc domain, wherein the iCID domain is in the format of an Fab comprising VH-CH1 and VL-CL. The second CTCoS fusion protein comprises, from the N terminal to the C terminal, the αTTABD-DL-CoS1-DL-Fc-DL-CoS2, wherein the αTTABD and the first co-stimulatory domains take the format of an scFv. The second co-stimulatory domain is a ligand linked to the C terminus of the second heterodimerization Fc domain.

In some embodiments, the first CTCoS fusion protein comprises, from the N terminal to the C terminal, CoS1-DL-iCID-Fc domain, wherein the iCID domain is in the format of an Fab comprising VH-CH1 and VL-CL, and the first co-stimulatory domain is a ligand (such as 41BBL trimer) linked to the N terminus of VH-CH1 and VL-CL. The second CTCoS fusion protein comprises, from the N terminal to the C terminal, the αTTABD-DL-CoS2-DL-Fc or CoS-DL-αTTABD-DL-Fc, wherein the αTTABD and the second co-stimulatory domains take the format of an scFv.

### 10. Formats of CC and CTTCoS Components of the BrighT-LITE Complexes

As generally outlined herein, the invention provides pairs of binding proteins (e.g. a CC binding protein and a CTTCoS binding protein) that together, in the presence of a CID-SM, form a T cell engaging complex. An exemplary configuration for Format 3 is depicted in FIG. 3. Generally, each binding protein is in turn made up of either two fusion proteins (that together form either a CC binding protein or a CTTCoS binding protein), or a monomeric fusion polypeptide as outlined below. As will be appreciated by those in the art, the CC binding proteins and the CTTCoS binding proteins can each be independently selected from monomeric fusion polypeptides, homodimeric fusion proteins and heterodimeric fusion proteins.

In one aspect, a composition comprises a CTTCoS heterodimeric binding protein comprising: a) a first CTCoS fusion protein comprising: i) a first iCID domain; ii) optional domain linker(s); iii) a first anti-tumor targeting antigen binding domain (αTTABD); iv) a first heterodimerization Fc domain; and b) a second CTCoS fusion protein comprising: i) a T-cell co-stimulatory receptor binding domain (CoS); ii) optional domain linker(s); iii) a second αTTABD; and iv) a second heterodimerization Fc domain.

In some embodiments, the first and/or antigen tumor targeting agent may be EpCAM. In some embodiments, the αTTABD may comprise the composition comprising the EpCAM binding domain described herein.

In some embodiments, the first iCID domain may comprise a composition comprising an indinavir binding domain. In some embodiments, the first iCID domain may comprise a composition comprising an indinavir-complex binding domain.

In some embodiments, the composition further comprises a second CT binding protein comprising the second iCID domain, wherein the CC binding protein further comprises a third iCID domain that is identical to the first iCID domain.

In one aspect, a co-stimulatory dual targeting T-cell ligand induced transient engager (dual BrighT-LITE) composition comprises: a) a CC binding protein; and b) a CTTCoS binding protein; wherein in the presence of indinavir said first and second iCID domains form a complex of said first iCID domain-said indinavir-said second iCID domain, such that said BrighT-LITE composition binds both CD3 and said tumor, wherein one of said first iCID and said second iCID is the indinavir binding domain described herein and the other is the indinavir-complex binding domain described herein, and wherein in the presence of indinavir said first and second iCID domains form a complex of said first iCID domain-indinavir-said second iCID domain, such that said T-LITE composition will bind both CD3 and said tumor. In some embodiments, the composition further comprises a second CT binding protein comprising the second iCID domain, wherein the CC binding protein further comprises a third iCID domain that is identical to the first iCID domain.

a. CC Binding Proteins

Accordingly, the present invention provides CC fusion polypeptides that form the CC binding protein(s) of the invention. The CC binding proteins of this format are the same as those for Format 1 or 2. Each CC binding protein contains a first CID domain, and an anti-CD3 ABD. In some embodiments, the CC binding protein does not contain an Fc domain, such as direct fusion of a first iCID domain and an αCD3-ABD. Both the iCID domain and the αCD3-ABD can take the format of an scFv, a Fab, an scFab or a single domain antibody such as the VHH of a camelid derived single domain antibody. In some embodiments, the CC binding protein contains an Fc domain. In some cases, the CC binding protein is monomeric. It can include an iCID domain directly fused to an αCD3-ABD, or it can also rely on the use of a monomeric Fc domain, as more fully outlined below. In some embodiments, the CC binding protein comprises a first and a second CC fusion polypeptide, that come together as dimers, either heterodimeric or homodimeric, to provide the αCD3-ABD functionally coupled to an iCID domain. In some embodiments, the iCID domain may be linked with another iCID domain via an optional domain linker as described above.

### (i) Monomeric CC Fusion Polypeptides

In some embodiments, the CC binding protein is monomeric and relies on the use of a monomeric IgG4 Fc domain. In some embodiments, the CC binding proteins are monomeric proteins comprising an iCID domain, an αCD3-ABD, optional domain linker(s) and an IgG4 monomeric Fc domain. The CC binding polypeptide can be a fusion polypeptide with a structure selected from the group, from N- to C-terminal: iCID domain-optional domain linker-αCD3-ABD-optional domain linker-Fc domain; αCD3-ABD-optional domain linker-iCID domain-optional domain linker-Fc domain; iCID domain-optional domain linker-Fc domain-optional domain linker-αCD3-ABD; αCD3-ABD-optional domain linker-Fc domain-optional domain linker-iCID domain; Fc domain-optional domain linker-αCD3-ABD-optional domain linker-iCID domain; Fc domain-optional domain linker-iCID domain-optional domain linker-αCD3-ABD; and iCID domain-optional domain linker-iCID domain-optional domain linker-αCD3 ABD-optional domain linker-Fc domain. Either or both of the iCID and αCD3-ABD can take any one of the formats including Fab, scFv, scFab, a single domain antibody such as the VHH of camelid derived single domain antibody.

In some instances, the selected arrangements of domains of the monomeric CC binding protein employed in the T-LITE composition provide an improvement, e.g., in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

### (ii) Dimeric CC Binding Proteins

In some embodiments, the CC binding protein comprises a first and a second CC fusion polypeptide, that come together as dimers, either heterodimeric or homodimeric, to provide the αCD3-ABD functionally coupled to an iCID domain. In these embodiments, the CC binding proteins rely on the use of Fc domains that are dimers, either heterodimeric Fc domains or homodimeric Fc domains.

In some embodiments, the CC binding proteins are CC heterodimeric binding proteins that use heterodimerization variants in the Fc domains. Thus, in some embodiments, the CC binding protein comprises a first and a second CC fusion polypeptide, wherein one of the first and second CC fusion polypeptides contains the αCD3-ABD and the other the iCID domain. In some embodiments, the CC binding protein comprises a first CC fusion polypeptide which contains both the αCD3-ABD and the iCID domain, and a second CC fusion polypeptide comprising an empty Fc domain. In these embodiments, the first and second CC fusion polypeptides can have the structures (from N- to C-terminal, with “DL” standing for “domain linker”) shown in Table 4. In some embodiments, each of the CC fusion polypeptide having one iCIDs may further comprise another iCID linked to the iCID via an optional DL, for example, as shown in 37-40 in Table 4. The iCIDs in the same fusion polypeptide may be identical. In some embodiments, the iCIDs in the same fusion polypeptide may be different. The DLs in the same fusion polypeptide may be different. In some embodiments, the DLs in the same fusion polypeptide may be same.

As discussed herein, each of the iCID domains and αCD3-ABD domains of Table 4 can be selected from a Fab, an scFab, an scFvs or a single domain antibody such as the VHH of camelid derived single domain antibody. The Fc domains in the first CC fusion polypeptide and second CC fusion polypeptide heterodimerize with each other. The iCID domain(s) in the first CC fusion polypeptide and/or second CC fusion polypeptide can be selected from either half of the iCID domain pairs described herein. Exemplary formats are illustrated in FIGS. 12 and 13A-13B.

In some embodiments, the CC binding proteins are CC homodimeric binding proteins that use standard Fc domains that self-assemble to form homodimers. In some embodiments, one of either of the iCID domain or the αCD3-ABD is formed using the VH and VL of a traditional, tetrameric antibody, and the other is attached to either the N- or C-terminus of the light chain or the N-terminus of the heavy chain.

In some embodiments, one of either of the iCID domain or the αCD3-ABD is formed using the VH and VL of a traditional, tetrameric antibody, and the other is attached to C-terminus of Fc domain. For example, the iCID domain can take a Fab format, and the αCD3-ABD can take an scFv format attached to the C-terminus of the Fc domain. Alternatively, the αCD3-ABD can take a Fab format, and the iCID domain can take an scFv format attached to the C-terminus of the Fc domain. In some embodiments, both the iCID domain and the αCD3-ABD take the format of an scFv or scFab. From N- to C-terminal, the CC binding protein comprises iCID domain-optional domain linker-αCD3-ABD-optional domain linker-homodimeric Fc domain or αCD3-ABD-optional domain linker-iCID domain-optional domain linker-homodimeric Fc domain.

In some instances, the selected arrangements of domains of the dimeric CC binding protein employed in the BrighT-LITE composition provide for an improvement, e.g. in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

(iii) Useful CC Heterodimeric Binding Proteins

As discussed herein, useful CC heterodimeric binding proteins are generally shown in FIGS. 3, 12A-12G and 13A-13C as discussed below.

The CID domain and αCD3 ABD can take various formats including an Fab, an scFv, an scFab, and a single domain antibody as described herein above. In some embodiments, both the iCID domain and αCD3 ABD take the format of an scFv. In some embodiments, the iCID domain takes the format of an Fab and the αCD3 ABD take the format of an scFv as shown in FIGS. 12 and 13A-13B. In some embodiments, the iCID domain takes the format of an scFab and the αCD3 ABD take the format of an scFv as shown in FIGS. 12 and 13A-13B. In some embodiments, the iCID domain takes the format of a single domain antibody and the αCD3 ABD take the format of an scFv.

In some embodiments, the iCID domain takes the format of a Fab and the αCD3 ABD take the format of an Fab. In some embodiments, the iCID domain takes the format of an scFab and the αCD3 ABD take the format of an Fab. In some embodiments, the iCID domain takes the format of an scFv and the αCD3 ABD take the format of an Fab. In some embodiments, the iCID domain takes the format of a single domain antibody and the αCD3 ABD take the format of a Fab.

In some embodiments, the iCID domain takes the format of an Fab and the αCD3 ABD take the format of an scFab. In some embodiments, the iCID domain takes the format of an scFab and the αCD3 ABD take the format of an scFab. In some embodiments, the iCID domain takes the format of an scFv and the αCD3 ABD take the format of an scFab. In some embodiments, the iCID domain takes the format of a single domain antibody and the αCD3 ABD take the format of an scFab.

In some embodiments, the iCID domain takes the format of an Fab and the αCD3 ABD take the format of a single domain antibody. In some embodiments, the iCID domain takes the format of an scFab and the αCD3 ABD take the format of a single domain antibody. In some embodiments, the iCID domain takes the format of an scFv and the αCD3 ABD take the format of a single domain antibody. In some embodiments, the iCID domain takes the format of a single domain antibody and the αCD3 ABD take the format of a single domain antibody.

In another aspect, the CC heterodimeric binding proteins comprises a Fc fusion protein and an empty Fc domain. The Fc fusion protein comprises an iCID domain, an αCD3 ABD, a first heterodimerization Fc domain and one or more optional linkers. The empty Fc domain contains a second heterodimerization Fc domain which heterodimerizes with the first heterodimerization Fc domain.

The iCID domain and αCD3 ABD can take various formats including an Fab, an scFv, an scFab, or a single domain antibody as described herein above. In some embodiments, the iCID takes the Fab format, and the αCD3 ABD takes the format of an Fab, an scFv, an scFab, or a single domain antibody. In some embodiments, the iCID takes the scFab format, and the αCD3 ABD takes the format of an Fab, an scFv, an scFab, or a single domain antibody. In some embodiments, the iCID takes the scFv format, and the αCD3 ABD takes the format of an Fab, an scFv, an scFab, or a single domain antibody. In some embodiments, the iCID takes the single domain antibody format, and the αCD3 ABD takes the format of an Fab, an scFv, an scFab, or a single domain antibody.

From N to C terminus, the Fc fusion protein can have configurations, such as iCID domain-optional domain linker-αCD3 ABD-optional domain linker-Fc, first iCID domain-optional domain linker-second iCID domain-optional domain linker-αCD3 ABD-optional domain linker-Fc, αCD3 ABD-optional domain linker-iCID domain-optional domain linker-Fc, αCD3 ABD-optional domain linker-first iCID domain-optional domain linker-second iCID domain-optional domain linker-Fc, αCD3 ABD-optional domain linker-Fc-optional domain linker-iCID domain, αCD3 ABD-optional domain linker-Fc-optional domain linker-first iCID domain-optional domain linker-second iCID domain, iCID domain-optional domain linker-Fc-optional domain linker-αCD3 ABD, and first iCID domain-optional domain linker-second iCID domain-optional domain linker-Fc-optional domain linker-αCD3 ABD. Further, from N to C terminus, the Fc fusion protein can have configurations, such as Fc-linker-iCID domain-optional domain linker-CD3 ABD, Fc-optional domain linker-CD3 ABD-optional domain linker-iCID domain, iCID domain-optional domain linker-Fc-liker-CD3 ABD, Fc-optional domain linker-first iCID domain-optional linker-second iCID domain-optional linker-CD3 ABD, Fc-Linker-CD3 ABD-optional domain linker-first iCID domain-optional domain linker-second iCID domain, and first iCID domain-optional domain linker-second iCID domain-optional domain linker-Fc-liker-CD3 ABD. Additionally, from N to C terminus, the Fc fusion protein can have configurations, such as iCID domain-optional domain linker-Fc, Fc-optional domain linker-iCID domain, CD3 ABD-optional domain linker-Fc, Fc-optional domain linker-CD3 ABD, first iCID domain-optional domain linker-Fc-optional domain linker-second iCID domain, first iCID domain-optional domain linker-second iCID domain-optional domain linker-Fc, Fc-optional domain linker-first iCID domain-optional domain linker-second iCID domain, CD3 ABD-optional domain linker-Fc, Fc-optional domain linker-CD3 ABD, first iCID domain-optional domain linker-second iCID domain-optional domain linker-Fc, and Fc-optional domain linker first iCID domain-optional domain linker-second iCID domain. The first and second iCID domains may be identical.

In one aspect, a composition described herein comprises a CC heterodimeric binding protein comprising; i) a first CC fusion protein comprising: 1) a first indinavir chemically induced dimerization (iCID) domain; 2) an optional linker; and 3) a first heterodimerization Fc domain; and ii) a second CC fusion protein comprising: 1) an anti-CD3 antigen binding domain (ABD; αCD3-ABD); 2) an optional domain linker, and 3) a second heterodimerization Fc domain. In some embodiments, the first CC fusion protein may further comprise a second iCID domain linked to the first iCID domain via an optional linker. The first and second iCID domains may be identical.

b. CTTCoS Binding Proteins

The invention provides CTTCoS binding proteins. Each CTCoS binding protein comprises an iCID domain, two or more anti-TTABD (αTTABD), and a T cell co-stimulatory domain. In some embodiments, the CTTCoS binding proteins comprises an Fc domain. In some embodiments, CTCoS binding proteins comprise a first and a second Fc fusion proteins, that come together as dimers, for example, heterodimerically to provide functional coupling of the two or more αTTABD, co-stimulatory domain and iCID domain. Any of the iCID domain, the αTTABDs and the co-stimulatory domain can take the format of an scFv, a Fab, an scFab or a single domain antibody such as the VHH of camelid derived single domain antibody.

### (i) Dimeric CTCoS Binding Proteins

In some embodiments, the CTCoS binding protein comprises a first and a second CTCoS fusion proteins, that come together heterodimerically to provide the functional coupling of the two or more αTTABDs, co-stimulatory domain and CID domain. In these embodiments, the CTCoS binding proteins rely on the use of heterodimerization variants in the Fc domains. Thus, in some embodiments, the CTCoS binding protein comprises a first and a second CTCoS fusion protein, wherein one of the first and second CTTCoS fusion protein contains the CID domain and at least one of the two or more αTTABDs, and the other CTCoS fusion protein contains the co-stimulatory domain and at least one of the two or more αTTABDs. In some instances, the first and second co-stimulatory domains are identical. In some instances, the two or more αTTABDs bind to the same tumor targeting antigen. In some instances, each of the two or more αTTABDs binds to different tumor targeting antigens.

Table 7 provides exemplary formats of the first and second CTTCoS fusion protein that can be coupled to form a BrighT-LITE in the presence of an iCID small molecule (“DL” standing for “optional domain linker” and “CoS” standing for “co-stimulatory domain”). In some embodiments, the CTCoS fusion protein comprising an iCID domain may further comprise another iCID domain linked to the iCID domain via an optional domain linker as described above for Format 1 above. In some embodiments, the CTCoS fusion protein comprising a αTTABD may further comprise another αTTABD linked to the αTTABD via an optional domain linker as described above for Format 1 or 2 above.

TABLE 7

| | First CTTCoS fusion protein | Second CTTCoS fusion protein |
|---|---|---|
| | (N- to C- terminal) | (N- to C- terminal) |
| 1 | αTTABD-DL-iCID-DL-Fc domain | αTTABD-DL-COS-DL-Fc domain |
| 2 | αTTABD-DL-iCID-DL-Fc domain | CoS-DL- αTTABD-DL-Fc domain |
| 3 | iCID-DL-αTTABD-DL-Fc domain | αTTABD-DL-CoS-DL-Fc domain |
| 4 | iCID-DL-αTTABD-DL-Fc domain | CoS-DL- αTTABD-DL-Fc domain |
| 5 | αTTABD-DL-iCID-DL-Fc domain | Fc domain-DL-αTTABD-DL-CoS |
| 6 | αTTABD-DL-iCID-DL-Fc domain | Fc domain-DL-CoS-DL-αTTABD-DL |
| 7 | iCID-DL-αTTABD-DL-Fc domain | Fc domain-DL-αTTABD-DL-CoS |
| 8 | iCID-DL-αTTABD-DL-Fc domain | Fc domain-DL-CoS-DL-αTTABD-DL |
| 9 | Fc domain-DL-αTTABD-DL-iCID | αTTABD-DL-CoS-DL-Fc domain |
| 10 | Fc domain-DL-αTTABD-DL-iCID | CoS-DL- αTTABD-DL-Fc domain |
| 11 | Fc domain-DL-iCID-DL-αTTABD-DL | αTTABD-DL-CoS-DL-Fc domain |
| 12 | Fc domain-DL-iCID-DL-αTTABD-DL | CoS-DL- αTTABD-DL-Fc domain |
| 13 | Fc domain-DL-αTTABD-DL-iCID | Fc domain-DL-αTTABD-DL-CoS |
| 14 | Fc domain-DL-αTTABD-DL-iCID | Fc domain-DL-CoS-DL-αTTABD-DL |
| 15 | Fc domain-DL-iCID-DL-αTTABD-DL | Fc domain-DL-αTTABD-DL-CoS |
| 16 | Fc domain-DL-iCID-DL-αTTABD-DL | Fc domain-DL-CoS-DL-αTTABD-DL |

As discussed herein, each of the iCID domains, αTTABDs, and co-stimulatory domain of Table 7 can be selected from a Fab, an scFab, an scFvs or a single domain antibody such as the VHH of camelid derived single domain antibody. The Fc domains in the first CTTCoS fusion protein and second CTTCoS fusion protein heterodimerize with each other. The iCID domain in the first CTTCoS fusion protein can be selected from one half of the iCID domain pairs described herein. The iCID domain in the second CTTCoS fusion protein can be selected from the other half of the CID domain pairs.

In some instances, the selected arrangements of domains of the dimeric CTTCoS binding proteins employed in the BrighT-LITE composition provide an improvement, e.g. in synthesis, stability, affinity or effector function, over other structures disclosed herein or known in the art. In some cases, a 2-fold, 3-fold, or 4-fold increase in, e.g. synthesis, stability, affinity, or effector activity is observed. In some cases, a 5-fold, 6-fold, 7-fold, 8-fold, 9-fold, 10-fold or higher increase in, e.g. stability, affinity, or effector activity, is observed.

### (ii) Useful CTTCoS Heterodimeric Binding Proteins

In some embodiments, the CTTCoS heterodimeric binding proteins comprising a first CTTCoS fusion protein and a second CTTCoS fusion protein. The first CTTCoS fusion protein comprises, an iCID domain, a first αTTABD, and a first heterodimerization Fc domain. The second CTTCoS fusion protein comprises, a CoS, a second αTTABD, and a second heterodimerization Fc domain. In some embodiments, the first CTTCoS fusion protein comprises, from the N terminal to C terminal, αTTABD-optional domain linker-iCID-optional domain linker-Fc domain. The second CTTCoS fusion protein comprises, from the N terminal to C terminal, αTTABD-optional domain linker-CoS-optional domain linker-Fc domain. The iCID domain and the αTTABD in these embodiments can take various formats including a Fab, an scFv, an scFab, and a single domain antibody as described herein. The CoS in these embodiments can be antibody fragment or a ligand as described herein. When the CoS is an antibody fragment, it can take various formats including a Fab, an scFv, an scFab, and a single domain antibody as described herein. For example, in some embodiments, the iCID domain, αTTABDs, and CoS take the format of an scFv. In some embodiments, the iCID domain takes the format of a Fab, and the αTTABDs and CoS take the format of an scFv as shown in FIG. 14. In some embodiments, the iCID domain takes the format of an scFab and the αTTABDs and CoS take the format of an scFv. In some embodiments, the iCID domain and CoS take the format of a Fab, and the αTTABDs take the format of an scFv. In some embodiments, the iCID domain and CoS take the format of an scFab, and the αTTABDs take the format of an scFv. In some embodiments, the iCID is a single domain molecule (e.g., an antibody domain that binds to indinavir or recognizes the complex of indinavir or the variants thereof), and the αTTABDs and CoS take the format of an scFv. In some embodiments, the iCID is a single domain molecule (e.g., BCl-2 or the variants thereof), and the αTTABDs and CoS take the format of an scFab. In some embodiments, the iCID is a single domain molecule, the CoS take the format of a Fab, and the αTTABDs take the format of an scFv. In some embodiments, the iCID is a single domain molecule, the CoS taked the format of an scFab, and the αTTABDs take the format of an scFv.

In some embodiments, the CTTCoS heterodimeric binding proteins comprising a first CTTCoS fusion protein and a second CTTCoS fusion protein. The first CTTCoS fusion protein comprises, an iCID domain, a first αTTABD, and a first heterodimerization Fc domain. The second CTTCoS fusion protein comprises, a CoS, a second αTTABD, and a second heterodimerization Fc domain. In some embodiments, the first CTTCoS fusion protein comprises, from the N terminal to C terminal, iCID-optional domain linker-αTTABD-optional domain linker-Fc domain. The second CTTCoS fusion protein comprises, from the N terminal to C terminal, CoS-optional domain linker-αTTABD-optional domain linker-Fc domain. The iCID domain and the αTTABD in these embodiments can take various formats including a Fab, an scFv, an scFab, and a single domain antibody as described herein. The CoS in these embodiments can be an antibody fragment or a ligand as described herein. When the CoS is an antibody fragment, it can take various formats including a Fab, an scFv, an scFab, and a single domain antibody as described herein. For example, in some embodiments, the iCID domain, αTTABDs, and CoS take the format of an scFv. In some embodiments, the αTTABDs take the format of a Fab, and the iCID and CoS take the format of an scFv. In some embodiments, the αTTABDs takes the format of an scFab, and the iCID and CoS take the format of an scFv. In some embodiments, the αTTABDs takes the format of an scFab, and the iCID and CoS take the format of an scFab. In some embodiments, the αTTABDs takes the format of an scFv, and the iCID and CoS take the format of an scFab. In some embodiments, the iCID is a single domain molecule, and the αTTABDs and CoS take the format of an scFv. In some embodiments, the iCID is a single domain molecule, the αTTABDs take the format of a Fab, and the CoS takes the format of an scFv as shown in FIG. 14. In some embodiments, the iCID is a single domain molecule, the αTTABDs take the format of an scFab, and the CoS takes the format of an scFv. In some embodiments, the iCID is a single domain molecule, the αTTABDs take the format of an scFab, and the CoS takes the format of an scFab. In some embodiments, the iCID is a single domain molecule, the αTTABDs take the format of an scFv, and the CoS take the format of an scFab.

### 11. Other Formats of the BrighT-LITEs of the Invention

As outlined herein, the BrighT-LITEs of the invention are made up of two (or more) different binding proteins, at least one CTTCoS binding protein and at least one CC binding protein, which can be combined in various combinations. In the presence of an iCID small molecule, the iCID domains of the CC and CTCoS binding proteins form a complex, such that the BrighT-LITE™ compositions will bind to both CD3 and tumor targeting antigen(s), becoming active T cell engaging complexes.

Any one of the CC binding proteins described herein can be combined with any one of the CTCoS binding proteins described herein.

### 12. Improving Switchable Activation of the T-LITEs and BrighT-LITEs of the Invention

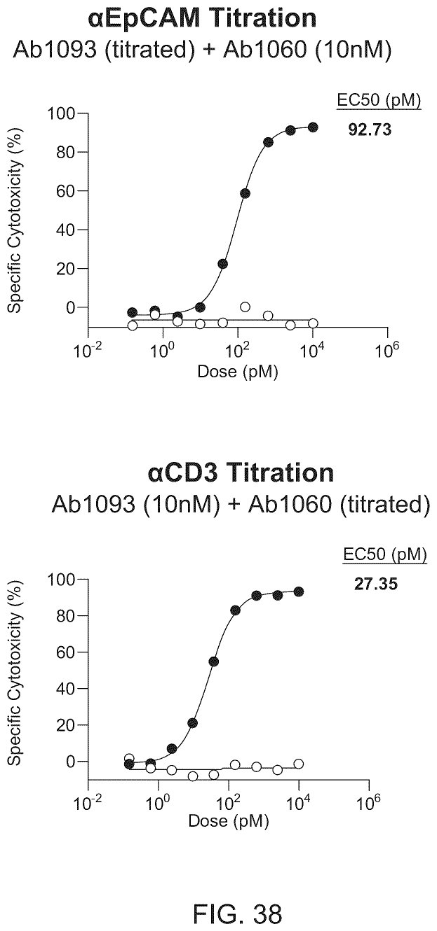

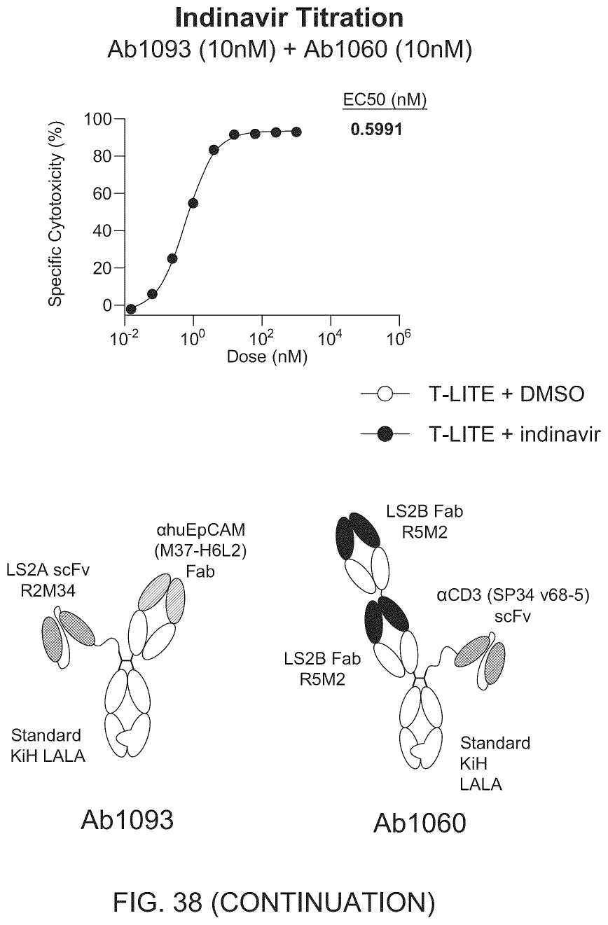

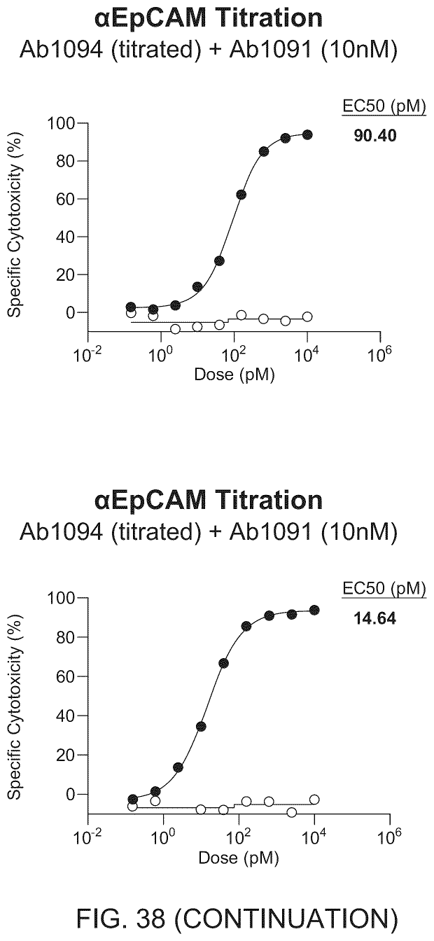

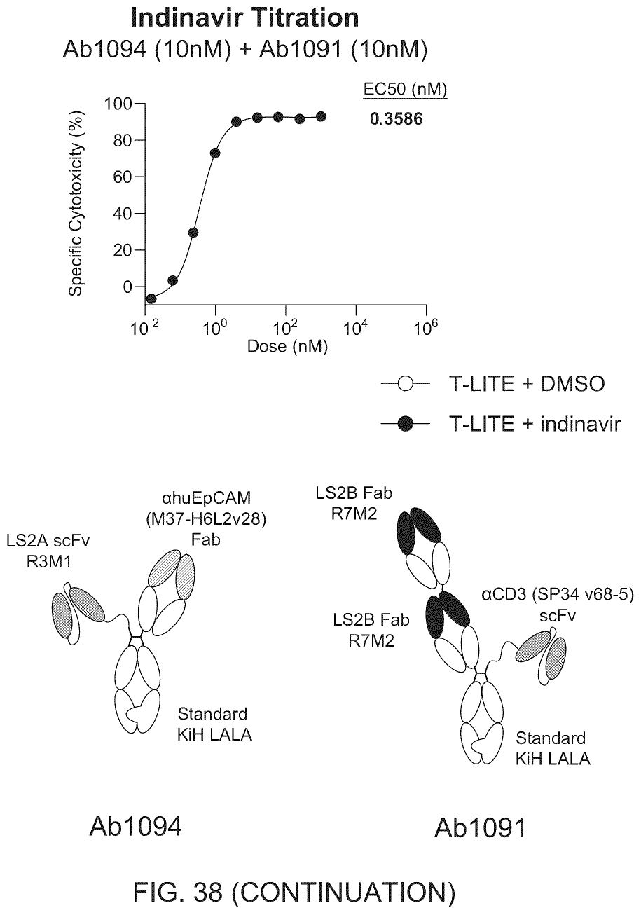

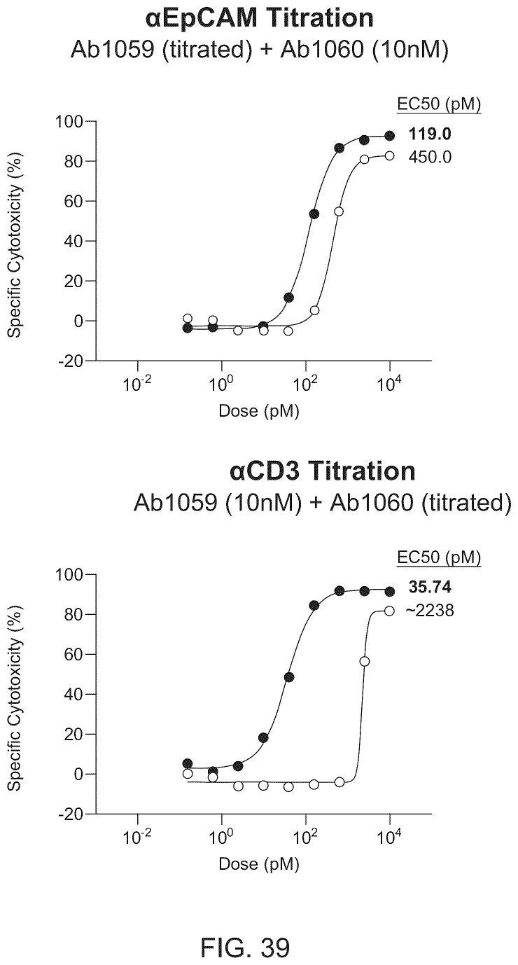

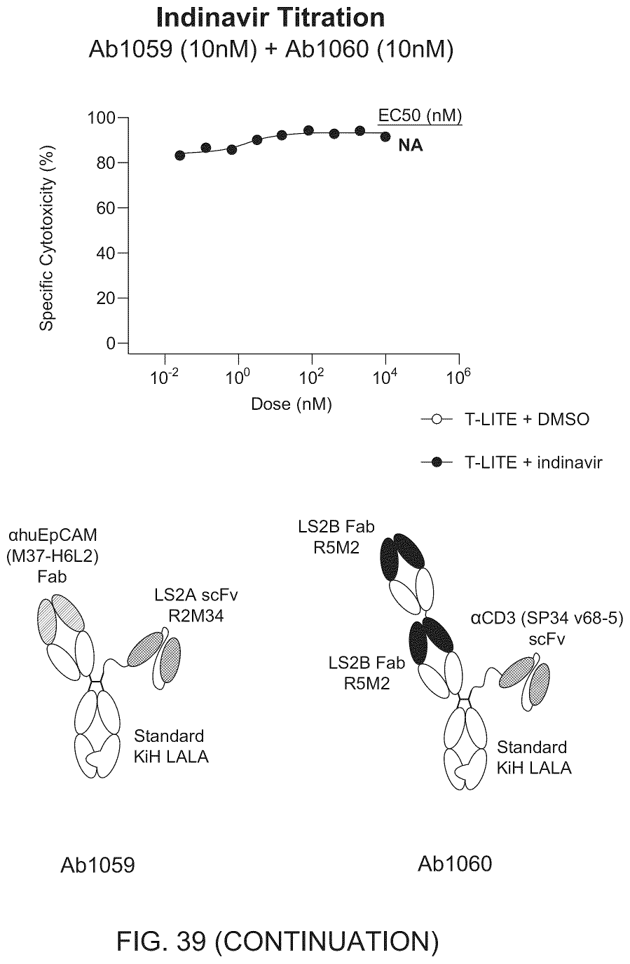

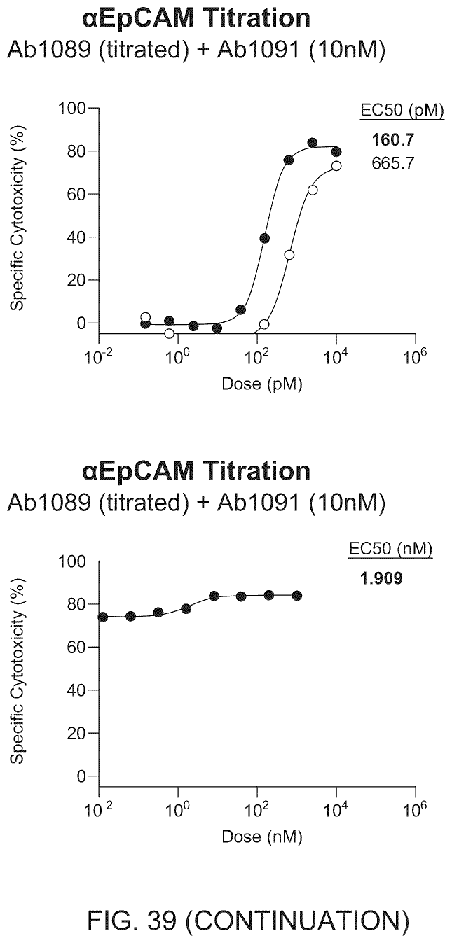

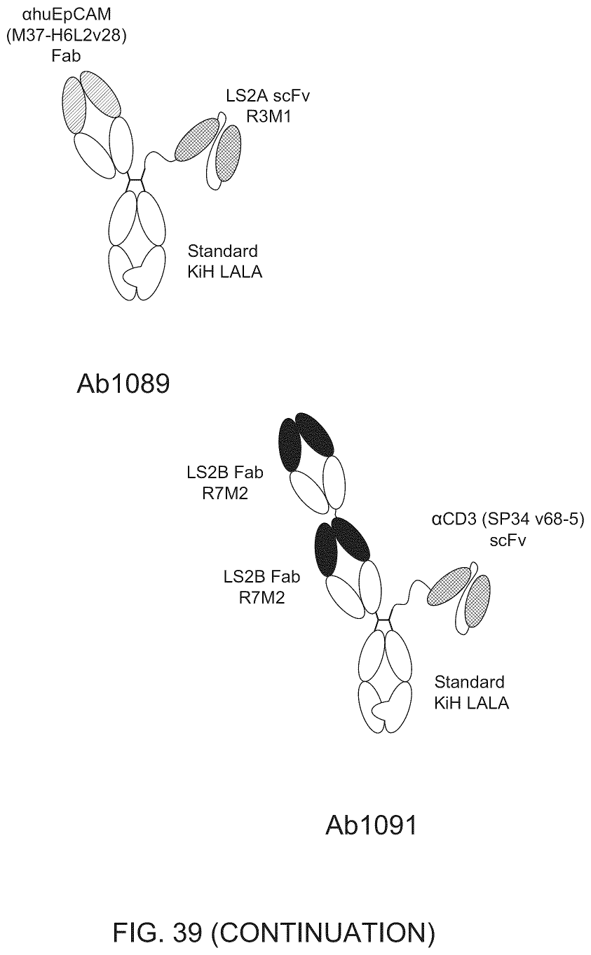

In another aspect, each of the CC and CT heterodimeric binding proteins having the iCID domain(s) as described herein comprises a first heterodimerization variant, and each of the CC and CT heterodimeric binding proteins having αCD3-ABD and αTTABD, respectively, as described herein comprises a second heterodimerization variant that corresponds to the first heterodimerization variant. In another aspect, each of the CC and CT heterodimeric binding proteins having the iCID domain(s) as described herein comprises a first Fc chain comprising a first heterodimerization variant, and each of the CC and CT heterodimeric binding proteins having αCD3-ABD and αTTABD, respectively, as described herein comprises an second Fc chain comprising a second heterodimerization variant that corresponds to the first heterodimerization variant. As shown in FIGS. 38 and 39, having Fc chains containing the same or similar heterodimerization variants in the CC and CT heterodimeric binding proteins comprising the iCID domain(s) may improve the switchable activation of the T-LITE, BrighT-LITE, dual targeting BrightT-LITES, or variants thereof as described herein. In some embodiments, the first heterodimerization variant is a hole variant, and the second heterodimerization variant is a knob variant.

In some embodiments, the first heterodimerization variant is a knob variant, and the second heterodimerization variant is a hole variant. In some embodiments, both CC and CT heterodimeric binding proteins having the iCID domain(s) have the same first heterodimerization variant. In some embodiments, both CC and CT heterodimeric binding proteins having αCD3-ABD and αTTABD, respectively, have the same second heterodimerization variant. The CT heterodimeric binding protein may be the CT Cos or CTTCos heterodimeric binding protein described herein.

In some embodiments, each of the CC and CT heterodimeric binding proteins having the iCID domain(s) as described herein comprises an Fc chain comprising a knob variant, and each of the corresponding CC and CT heterodimeric binding proteins having αCD3-ABD and αTTABD, respectively, comprises an Fc chain comprising a hole variant corresponding to the knob variant. In some embodiments, each of the CC and CT heterodimeric binding proteins having the iCID domain(s) as described herein comprises an Fc chain comprising a hole variant, and each of the corresponding CC and CT heterodimeric binding proteins having αCD3-ABD and αTTABD, respectively, comprises an Fc chain comprising a knob variant corresponding to the hole variant. Some examples of the knob and hole Fc sequences are disclosed in FIG. 10.

In some embodiments, the Fc domain described herein having a knob variant comprises a sequence having at least 85, 86, 87, 88, 89, 90, 91, 92, 93, 94, 95, 96, 97, 98, 99 and 100% identity to any one of SEQ ID NO: 3235, 3237, 3239, 3241, and 3243. In some embodiments, the Fc domain described herein having a hole variant comprises a sequence having at least 85, 86, 87, 88, 89, 90, 91, 92, 93, 94, 95, 96, 97, 98, 99 and 100% identity to any one of SEQ ID NO: 3236, 3238, 3240, 3242, and 3244. In some embodiments, the Fc domain described herein having a knob variant comprises the sequence of 3235. In some embodiments, the Fc domain described herein having a knob variant comprises the sequence of 3237. In some embodiments, the Fc domain described herein having a knob variant comprises the sequence of 3239. In some embodiments, the Fc domain described herein having a knob variant comprises the sequence of 3241. In some embodiments, the Fc domain described herein having a knob variant comprises the sequence of 3243. In some embodiments, the Fc domain described herein having a hole variant comprises the sequence of SEQ ID NO: 3236. In some embodiments, the Fc domain described herein having a hole variant comprises the sequence of SEQ ID NO: 3238. In some embodiments, the Fc domain described herein having a hole variant comprises the sequence of SEQ ID NO: 3240. In some embodiments, the Fc domain described herein having a hole variant comprises the sequence of SEQ ID NO: 3242. In some embodiments, the Fc domain described herein having a hole variant comprises the sequence of SEQ ID NO: 3244.

### C. Nucleic Acids, Expression Vectors, Host Cells

Nucleic acid compositions encoding the T-LITE™ and BrighT-LITE™ compositions described herein are provided, including polynucleotide molecules encoding each component of the CC and CTTCoS binding proteins described herein.

Expression vectors containing the nucleic acids, and host cells transformed with the nucleic acids and/or expression vectors are also provided. As will be appreciated by those in the art, the protein sequences depicted herein can be encoded by any number of possible nucleic acid sequences, due to the degeneracy of the genetic code.

In some embodiments, the polynucleotide molecules are provided as DNA constructs.

In some embodiments, the polynucleotide molecules encoding each monomeric fusion protein of the CC and CT binding protein are placed into different expression vectors. In some embodiments, the polynucleotide molecules encoding each monomeric fusion protein of the CC and CT binding protein are placed into a single expression vector.

In some embodiments, the polynucleotide molecules encoding each monomeric fusion protein of the CC binding protein are placed into a first single expression vector, and the polynucleotide molecules encoding each monomeric fusion protein of the CT binding protein are placed into a second single expression vector.

In some embodiments, the polynucleotide molecules encoding each fusion protein of the CC and CTCoS binding protein are placed into different expression vectors. In some embodiments, the polynucleotide molecules encoding each fusion protein of the CC and CTCoS binding protein are placed into a single expression vector.

In some embodiments, the polynucleotide molecules encoding each fusion protein of the CC binding protein are placed into a first single expression vector, and the polynucleotide molecules encoding each fusion protein of the CTCoS binding protein are placed into a second single expression vector.

In some embodiments, the polynucleotide molecules encoding each fusion protein of the CC and CTTCoS binding protein are placed into different expression vectors. In some embodiments, the polynucleotide molecules encoding each fusion protein of the CC and CTTCoS binding protein are placed into a single expression vector.

In some embodiments, the polynucleotide molecules encoding each fusion protein of the CC binding protein are placed into a first single expression vector, and the polynucleotide molecules encoding each fusion protein of the CTTCoS binding protein are placed into a second single expression vector.

Expression vectors, as is known in the art, can contain the appropriate transcriptional and translational control sequences, including, but not limited to, signal and secretion sequences, regulatory sequences, promoters, origins of replication, selection genes, etc.

Expression vectors can be transformed into host cells, where they are expressed to form the composition described herein. An appropriate host cell expression system includes but is not limited to bacteria, an insect cell, and a mammalian cell. Preferred mammalian host cells for expressing the recombinant antibodies according to at least some embodiments of the invention include Chinese Hamster Ovary (CHO cells), PER.C6, HEK293 and others as is known in the art.

In some embodiments, the CC and CT binding proteins are produced and isolated separately. The expression vector(s) comprising polynucleotide molecules encoding each monomeric fusion protein of the CC binding protein can be transformed into one host cell. Components of the CC binding proteins are expressed by the host cell, isolated and, optionally, further purified. The expression vector(s) comprising polynucleotide molecules encoding each monomeric fusion protein of the CT binding protein can be transformed into another host cell. Components of the CT binding proteins are expressed by the host cell, isolated and, optionally, further purified. In some embodiments, the CC and CT binding proteins are produced and isolated together. Accordingly, the expression vector(s) comprising polynucleotide molecules encoding each monomeric fusion protein of the CC binding protein and CT binding proteins can be transformed into a single host cell for protein expression and further isolation.

In some embodiments, the CC and CTCoS binding proteins are produced and isolated separately. The expression vector(s) comprising polynucleotide molecules encoding each fusion protein of the CC binding protein can be transformed into one host cell. Components of the CC binding proteins are expressed by the host cell, isolated and, optionally, further purified. The expression vector(s) comprising polynucleotide molecules encoding each fusion protein of the CTCoS binding protein can be transformed into another host cell. Components of the CTCoS binding proteins are expressed by the host cell, isolated and, optionally, further purified.

In some embodiments, the CC and CTCoS binding proteins are produced and isolated together. Accordingly, the expression vector(s) comprising polynucleotide molecules encoding each fusion protein of the CC binding protein and CTCoS binding protein can be transformed into a single host cell for protein expression and further isolation.

In some embodiments, the CC and CTTCoS binding proteins are produced and isolated separately. The expression vector(s) comprising polynucleotide molecules encoding each fusion protein of the CC binding protein can be transformed into one host cell. Components of the CC binding proteins are expressed by the host cell, isolated and, optionally, further purified. The expression vector(s) comprising polynucleotide molecules encoding each fusion protein of the CTTCoS binding protein can be transformed into another host cell. Components of the CTTCoS binding proteins are expressed by the host cell, isolated and, optionally, further purified.

In some embodiments, the CC and CTTCoS binding proteins are produced and isolated together. Accordingly, the expression vector(s) comprising polynucleotide molecules encoding each fusion protein of the CC binding protein and CTTCoS binding protein can be transformed into a single host cell for protein expression and further isolation.

In another aspect, the present disclosure relates to a T-cell ligand induced transient engager (T-LITE) composition comprising: a) a CC binding protein according to any of claims D1 to D7; and b) a CT binding protein according to any of claims E1 to E6; wherein one of said first iCID and said second iCID is an indinavir binding domain and the other is an indinavir-complex binding domain, wherein said indinavir binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4-D11, P7-D12, P1-C11, P4-F9, P6-HS, P2-D7, P3-FS, P3-C5, P8-F4, P8-C2, P7-F12, P2-H7, P1-B7, P2-D9, P6-E8, P3-B9, P1-G1, P1-D11, P8-E3, P4-E4, PS-D12, P3-F4, P6-H2, P1-E9, P8-D9, and P4-G11 (FIG. 49) and said indinavir-complex binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50); and wherein in the presence of indinavir said first and second iCID domains form a complex of said first iCID domain-indinavir-said second iCID domain, such that said T-LITE composition will bind both CD3 and said tumor. In another aspect, the present disclosure relates to a composition comprising a CTCoS heterodimeric binding protein comprising: a) a first CTCoS fusion protein comprising: i) a first iCID domain; ii) optional domain linker(s); and iii) a first heterodimerization Fc domain; and b) a second CTCoS fusion protein comprising: i) an anti-tumor target antigen binding domain (αTTABD); ii) optional domain linker(s); and iii) a second heterodimerization Fc domain; wherein one of said first and second CTCoS fusion proteins further comprises a co-stimulatory domain. In some embodiments, said first iCID domain is an indinavir binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4- D11, P7-D12, P1-C11, P4-F9, P6-HS, P2-D7, P3-FS, P3-C5, P8-F4, P8-C2, P7-F12, P2-H7, P1-B7, P2- D9, P6-E8, P3-B9, P1-G1, P1-D11, P8-E3, P4-E4, PS-D12, P3-F4, P6-H2, P1-E9, P8-D9, and P4-G11 (FIG. 49). In some embodiments, said first iCID domain is an indinavir binding domain comprising a VH domain and VL domain selected from the group consisting of the VH and VL domains of P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4- D11, P7-D12, P1-C11, P4-F9, P6-HS, P2-D7, P3-FS, P3-C5, P8-F4, P8-C2, P7-F12, P2-H7, P1-B7, P2- D9, P6-E8, P3-B9, P1-G1, P1-D11, P8-E3, P4-E4, PS-D12, P3-F4, P6-H2, P1-E9, P8-D9, and P4-G11 (FIG. 49). In some embodiments, the first iCID is an indinavir-complex binding domain comprising: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). In some embodiments, second iCID is an indinavir-complex binding domain comprising a VH domain and VL domain selected from the group consisting of the VH and VL domains of Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50). The present disclosure relates to a co-stimulatory T-cell ligand induced transient engager (BrighT-LITE) composition comprising: a) a CC binding protein according to any of claims D1 to D7; and the CTCoS binding protein; wherein in the presence of indinavir said first and second iCID domains form a complex of said first iCID domain-said indinavir-said second iCID domain, such that said BrighT-LITE composition binds both CD3 and said tumor, wherein one of said first iCID and said second iCID is an indinavir binding domain and the other is an indinavir-complex binding domain, wherein said indinavir binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4- D11, P7-D12, P1-C11, P4-F9, P6-HS, P2-D7, P3-FS, P3-C5, P8-F4, P8-C2, P7-F12, P2-H7, P1-B7, P2- D9, P6-E8, P3-B9, P1-G1, P1-D11, P8-E3, P4-E4, PS-D12, P3-F4, P6-H2, P1-E9, P8-D9, and P4-G11 (FIG. 49) and said indinavir-complex binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50); and wherein in the presence of indinavir said first and second iCID domains form a complex of said first iCID domain-indinavir-said second iCID domain, such that said T-LITE composition will bind both CD3 and said tumor. The present disclosure relates to a composition comprising a CTTCoS heterodimeric binding protein comprising: a) a first CTTCoS fusion protein comprising: i) a first iCID domain; ii) optional domain linker(s); iii) a first anti-tumor targeting antigen binding domain (αTTABD); iv) a first heterodimerization Fc domain; and b) a second CTTCoS fusion protein comprising: i) a T-cell co-stimulatory receptor binding domain (CoS); ii) optional domain linker(s); iii) a second αTTABD; and iv) a second heterodimerization Fc domain; wherein one of said first iCID and said second iCID is an indinavir binding domain and the other is an indinavir-complex binding domain, wherein said indinavir binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4-D11, P7-D12, P1-C11, P4-F9, P6-HS, P2-D7, P3-FS, P3-C5, P8-F4, P8-C2, P7-F12, P2-H7, P1-B7, P2-D9, P6-E8, P3-B9, P1-G1, P1-D11, P8-E3, P4-E4, PS-D12, P3-F4, P6-H2, P1-E9, P8-D9, and P4-G11 (FIG. 49) and said indinavir-complex binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50); and wherein in the presence of indinavir said first and second iCID domains form a complex of said first iCID domain-indinavir-said second iCID domain, such that said T-LITE composition will bind both CD3 and said tumor. The present disclosure relates to a co-stimulatory dual targeting T-cell ligand induced transient engager (dual BrighT-LITE) composition comprising: a) the CC binding protein; and the CTCoS binding protein; wherein in the presence of indinavir said first and second iCID domains form a complex of said first iCID domain-said indinavir-said second iCID domain, such that said BrighT-LITE composition binds both CD3 and said tumor, wherein one of said first iCID and said second iCID is an indinavir binding domain and the other is an indinavir-complex binding domain, wherein said indinavir binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from P7-F7, P7-E7, PS-HS, P2-D8, P3-E12, P6-C8, P7-G8, P6-D7, P6-DS, P3-A11, PS-C3, P3-HS, P7-H10, P7-C6, P2-C2, P2-E8, PS-E9, P3-H7, P7-D6, P6-D11, PS-A9, P4-C5, P3-H2, P1-H3, P1-B12, P4-G12, P2-A10, P4-D11, P7-D12, P1-C11, P4-F9, P6-HS, P2-D7, P3- FS, P3-C5, P8-F4, P8-C2, P7-F12, P2-H7, P1-B7, P2-D9, P6-E8, P3-B9, P1-G1, P1-D11, P8-E3, P4-E4, PS-D12, P3-F4, P6-H2, P1-E9, P8-D9, and P4-G11 (FIG. 49) and said indinavir-complex binding domain comprises: i) a variable heavy (VH) domain comprising a vhCDR1, vhCDR2, and vhCDR3 sequence; ii) a variable light (VL) domain comprising a vlCDR1, vlCDR2, and vlCDR3 sequence; wherein said vhCDR1, vhCDR2 and vhCDR3 sequences and said vlCDR1, vlCDR2, and vlCDR3 sequences are selected from the group consisting of the vhCDR1, vhCDR2 and vhCDR3 sequences and the vlCDR1, vlCDR2, and vlCDR3 sequences from Fab001, Fab002, Fab003, Fab004, Fab005, Fab006, Fab007, Fab008, Fab009, Fab0010, Fab0011, Fab0012, Fab0013, Fab0014, Fab0015, Fab0016, Fab0017, Fab0018, Fab0019, Fab0020, Fab0021, and Fab0022 (FIG. 50); and wherein in the presence of indinavir said first and second iCID domains form a complex of said first iCID domain-indinavir-said second iCID domain, such that said T-LITE composition will bind both CD3 and said tumor.

### D. Formulations

The T-LITE™ and BrighT-LITE™ compositions used in the practice of the foregoing methods can be formulated into pharmaceutical compositions comprising a carrier suitable for the desired delivery method. Suitable carriers include any material that when combined with the therapeutic composition retains the therapeutic function of the therapeutic composition and is generally non-reactive with the patient's immune system. Examples include, but are not limited to, any of a number of standard pharmaceutical carriers such as sterile phosphate buffered saline solutions, bacteriostatic water, and the like (see, generally, Remington's Pharmaceutical Sciences 16^{th} Edition, A. Osal., Ed., 1980). Acceptable carriers, excipients, or stabilizers are nontoxic to recipients at the dosages and concentrations employed and may include buffers.

In some embodiments, the CC and CT binding proteins are formulated together in the same container. In some embodiments, the CC and CT binding proteins are formulated separately in different containers.

In some embodiments, the iCID small molecule is formulated separately from the CC and CT binding proteins. The iCID small molecule can be incorporated into a variety of formulations for therapeutic administration, for example, by combination with appropriate, pharmaceutically acceptable carriers or diluents to achieve the desired state in the subject being treated.

In some embodiments, the CC and CTCoS binding proteins are formulated together in the same container. In some embodiments, the CC and CTCoS binding proteins are formulated separately in different containers.

In some embodiments, the iCID small molecule is formulated separately from the CC and CTCoS binding proteins. The iCID small molecule can be incorporated into a variety of formulations for therapeutic administration, for example, by combination with appropriate, pharmaceutically acceptable carriers or diluents to achieve the desired state in the subject being treated.

In some embodiments, the CC and CTTCoS binding proteins are formulated together in the same container. In some embodiments, the CC and CTTCoS binding proteins are formulated separately in different containers.

In some embodiments, the iCID small molecule is formulated separately from the CC and CTTCoS binding proteins. The iCID small molecule can be incorporated into a variety of formulations for therapeutic administration, for example, by combination with appropriate, pharmaceutically acceptable carriers or diluents to achieve the desired state in the subject being treated.

### E. Methods of Making and Using the Compositions

The T-LITE™ and BrighT-LITE™ compositions described herein can find use in a number of therapeutic applications. Usually, a patient is a human, but non-human mammals including transgenic mammals can also be treated.

A method of making compositions described herein comprises the following steps: 1) an immunization campaign, 2) validation of murine indinavir binders, 3) humanization of the murine indinavir binders, 4) generation of a composition described herein and 5) validation of a composition described herein.

In some embodiments, the CC and CT binding proteins are formulated and administered together to a patient. In some embodiments, the CC and CT binding proteins are formulated separately and administered together to a patient after pre-administration mixing of the two. In some embodiments, the CC and CT binding proteins are formulated separately and administered sequentially to a patient. The route of administration can be, for example, intravenous.

Administration of an iCID small molecule to the same patient induces association of the CC and CT binding proteins, bringing together the tumor targeting antigen binding domain with the T cell engaging domain and forming an active T cell engaging complex. The iCID small molecule can be administered before, simultaneously with, or after the administration of the T-LITE™ compositions. The iCID small molecule may be administered multiple times or at varying doses to modulate the activity of the T cell engaging complex. To maintain the activity of the T cell engaging complexes (i.e., the association of the tumor targeting antigen domain with the T cell engaging domain), the patient can be dosed regularly with the iCID small molecule. The frequency of dosing depends on the iCID small molecule's serum half-life. The route of administration of an iCID small molecule can be, for example, oral, intravenous, subcutaneous, or intratumoral.

In the event that the patient needs to stop the activity of the T cell engaging complexes quickly, for example, due to safety concerns, the patient would stop being dosed with the iCID small molecule. This leads to clearance of the iCID small molecule, disassociation of the CC binding protein from the CT binding protein, and decoupling of the T cell from the target cell.

In some embodiments, the CC and CTCoS binding proteins are formulated and administered together to a patient. In some embodiments, the CC and CTCoS binding proteins are formulated separately and administered together to a patient after pre-administration mixing of the two. In some embodiments, the CC and CTCoS binding proteins are formulated separately and administered sequentially to a patient. The route of administration can be, for example, intravenous.

Administration of an iCID small molecule to the same patient induces association of the CC and CTCoS binding proteins, bringing together the tumor targeting antigen binding domain with the T cell engaging domain and forming an active T cell engaging complex. The iCID small molecule can be administered before, simultaneously with, or after the administration of the BrighT-LITE™ compositions. The iCID small molecule may be administered multiple times or at varying doses to modulate the activity of the T cell engaging complex. To maintain the activity of the T cell engaging complexes (i.e., the association of the tumor targeting antigen domain with the T cell engaging domain), the patient can be dosed regularly with the iCID small molecule. The frequency of dosing depends on the iCID small molecule's serum half-life. The route of administration of an iCID small molecule can be, for example, oral, intravenous, subcutaneous, or intratumoral.

In the event that the patient needs to stop the activity of the T cell engaging complexes quickly, for example, due to safety concerns, the patient would stop being dosed with the iCID small molecule. This leads to clearance of the iCID small molecule, disassociation of the CC binding protein from the CTCoS binding protein, and decoupling of the T cell from the target cell.

In some embodiments, the CC and CTTCoS binding proteins are formulated and administered together to a patient. In some embodiments, the CC and CTTCoS binding proteins are formulated separately and administered together to a patient after pre-administration mixing of the two. In some embodiments, the CC and CTTCoS binding proteins are formulated separately and administered sequentially to a patient. The route of administration can be, for example, intravenous.

Administration of an iCID small molecule to the same patient induces association of the CC and CTTCoS binding proteins, bringing together the tumor targeting antigen binding domains with the T cell engaging domain and forming an active T cell engaging complex. The iCID small molecule can be administered before, simultaneously with, or after the administration of the BrighT-LITE™ compositions. The iCID small molecule may be administered multiple times or at varying doses to modulate the activity of the T cell engaging complex. To maintain the activity of the T cell engaging complexes (i.e., the association of the tumor targeting antigen domain with the T cell engaging domain), the patient can be dosed regularly with the iCID small molecule. The frequency of dosing depends on the iCID small molecule's serum half-life. The route of administration of an iCID small molecule can be, for example, oral, intravenous, subcutaneous, or intratumoral.

In the event that the patient needs to stop the activity of the T cell engaging complexes quickly, for example, due to safety concerns, the patient would stop being dosed with the iCID small molecule. This leads to clearance of the iCID small molecule, disassociation of the CC binding protein from the CTTCoS binding protein, and decoupling of the T cell from the target cell.

The methods described above enable a precise temporal control of the activity of a T cell engaging complex in a patient, and the method is applicable to treat patients suffering from a variety of tumorous diseases or conditions. For example, a T-LITE or a BrighT-LITE comprising an αCD19-ABD can be used to treat patients suffering from CD19 expressing tumors, for example, most of B cell malignancies including but not limited to acute lymphoblastic leukemia (ALL), chronic lymphocytic leukemia (CLL) and B cell lymphomas.

Similarly, a T-LITE or a BrighT-LITE comprising an αEpCAM-ABD can be used to treat patients suffering from EpCAM expressing tumors. A T-LITE or a BrighT-LITE comprising an αHER2-ABD can be used to treat patients suffering from HER2 expressing tumors. A T-LITE or a BrighT-LITE comprising an α CD20-ABD can be used to treat patients suffering from CD20 expressing tumors.

In one aspect, the present disclosure relates to a method of treating cancer in a subject, comprising administering, to the subject, any one of the compositions described herein. For example, the composition described herein, including, but not limited to, a T-LITE or a BrighT-LITE comprising an αCD3-ABD, can be used to treat patients suffering from EpCAM expressing tumors.

Administration of the compositions described herein may be done in a variety of ways, including, but not limited to intravenously or locally.

The dosing amounts and frequencies of administration are, in a preferred embodiment, selected to be therapeutically or prophylactically effective. As is known in the art, dosages for any one patient depends on many factors, the age, body weight, general health, sex, diet, time and route of administration, drug interaction and the severity of the condition may be necessary.

In one aspect, the present disclosure relates to use of any one of the compositions described herein for treating cancer. In another aspect, the present disclosure relates to a kit comprising any one of the compositions described herein. In some embodiments, the kit is for treating cancer. In another aspect, the cancer described herein is a EpCAM expressing tumor.

### Examples

### LS2B Engineering

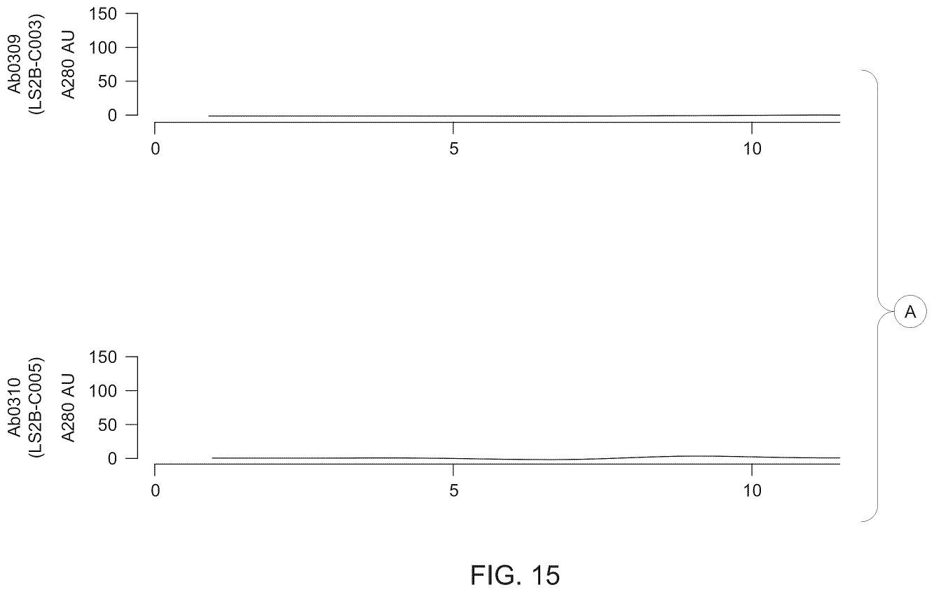

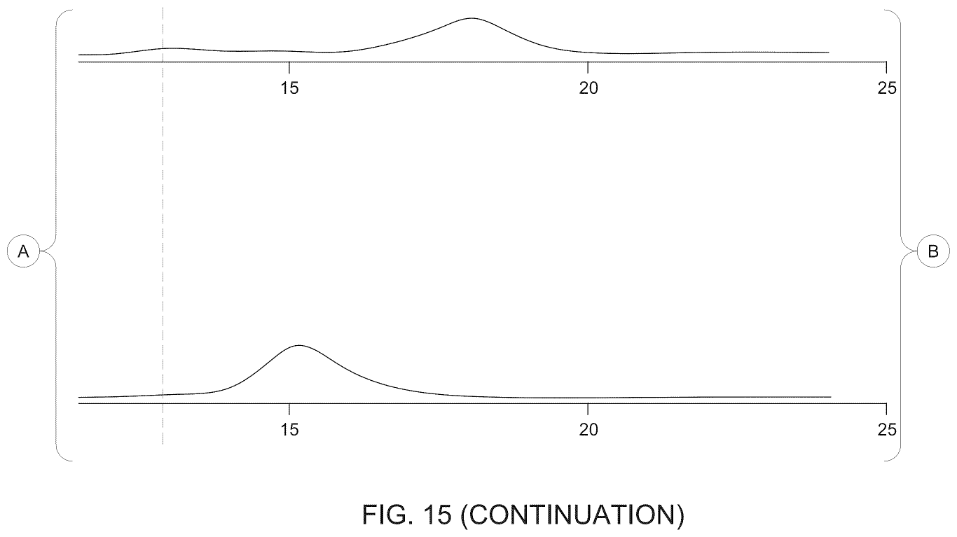

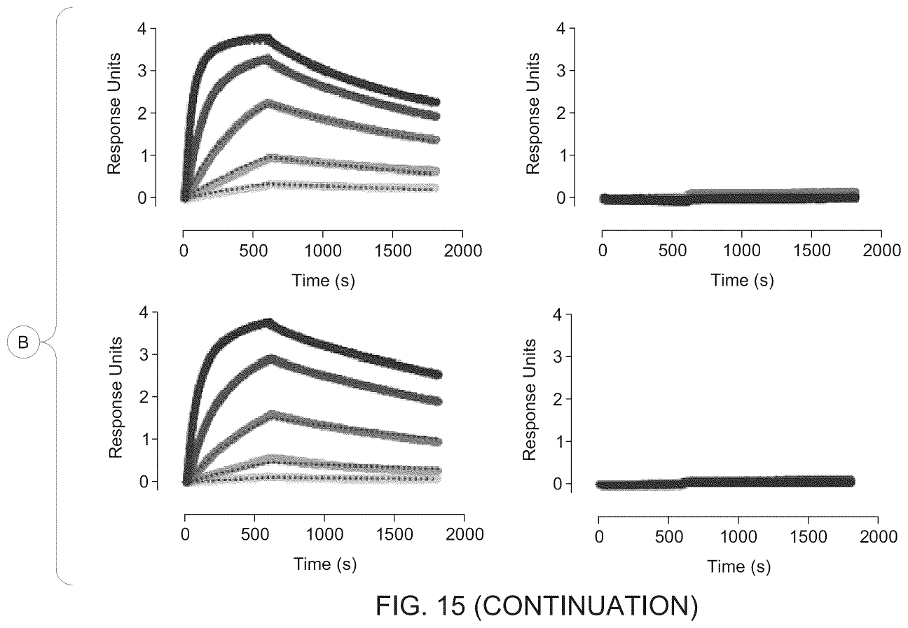

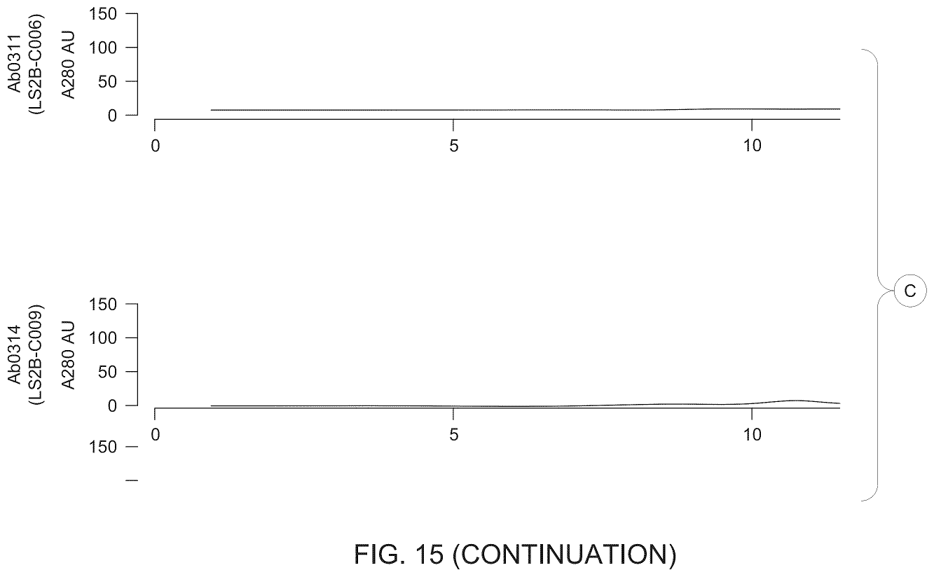

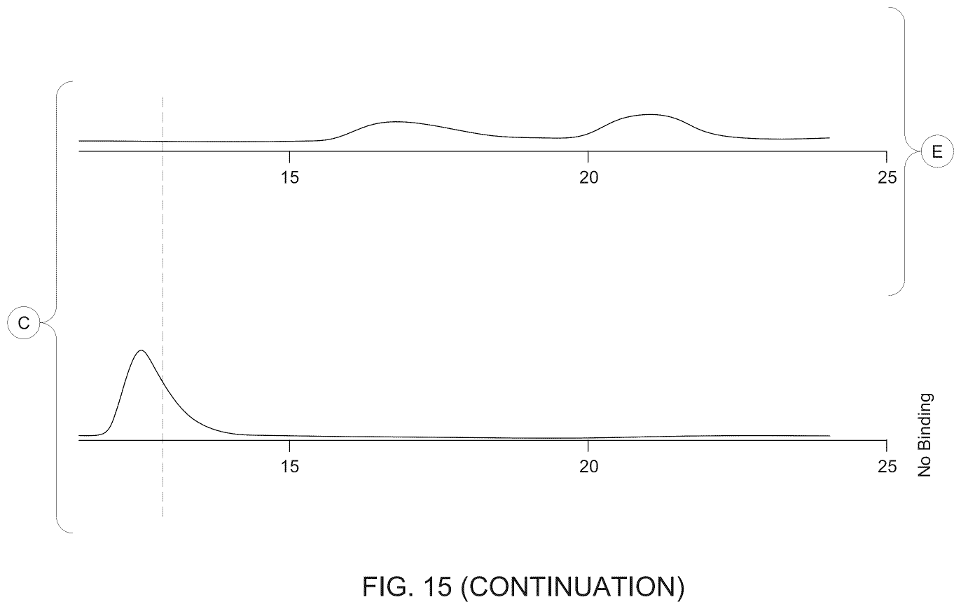

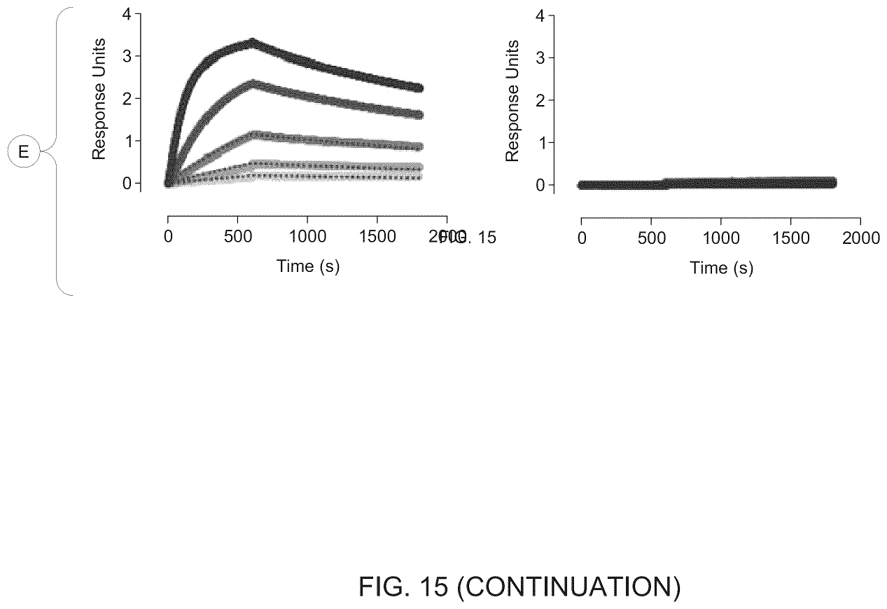

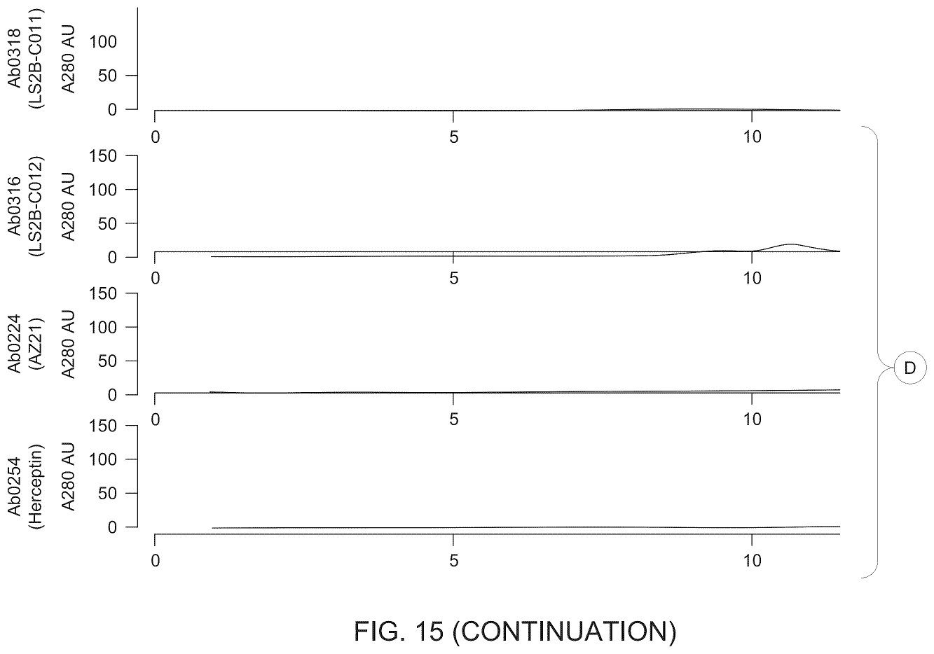

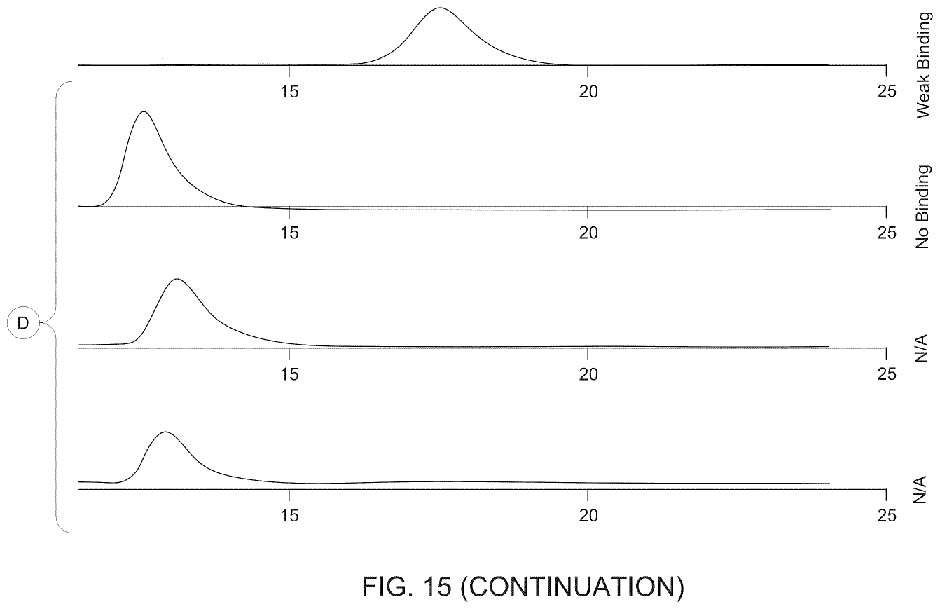

LS2B antibody clones derived from the phage display campaign were profiled by analytical UPLC-SEC using a Thermo Vanquish Flex HPLC with a MabPAC 4.6×150 mm SEC column equilibrated in 1×PBS-HCl at pH 7.0. Retention time of each clone was compared to Ab0254 to assess overall developability. Ab0310 (LS2B-C005) showed the best combination of binding and retention time (FIG. 15). However, the retention time was later than expected, suggesting that the hydrophobicity of the clone could be leading to interaction with the SEC column. This motivated protein engineering to ameliorate this liability.

In R1 of engineering, all Tyrosines and Tryptophans were individually mutated to Alanine or Serine to remove hydrophobicity as shown in Table 8 below.

TABLE 8

| | | | LS2A Ab0223 |
|---|---|---|---|
| Ab | Clone | Mutations | binding screen |
| Ab0272 | Clone5 | Parent for this set | ++++ |
| Ab0388 | R1M1 | HC: Y53S | ++++ |
| Ab0389 | R1M2 | HC: Y53A | ++++ |
| Ab0390 | R1M3 | HC: Y54S | ++ |
| Ab0391 | R1M4 | HC: Y54A | ++ |
| Ab0392 | R1M5 | HC: Y58S | ++++ |
| Ab0393 | R1M6 | HC: Y58A | ++++ |
| Ab0394 | R1M7 | HC: Y95S | +++ |
| Ab0395 | R1M8 | HC: Y95A | +++ |
| Ab0396 | R1M9 | HC: Y96S | ++ |
| Ab0397 | R1M10 | HC: Y96A | +++ |
| Ab0398 | R1M11 | HC: W98S | ++ |
| Ab0399 | R1M12 | HC: W98A | +++ |
| Ab0400 | R1M13 | HC: Y99S | ++ |
| Ab0401 | R1M14 | HC: Y99A | ++ |
| Ab0402 | R1M15 | HC: Y100aS | +++ |
| Ab0403 | R1M16 | HC: Y100aA | +++ |
| Ab0404 | RIM17 | HC: Y100bS | ++++ |
| Ab0405 | R1M18 | HC: Y100bA | ++++ |
| Ab0406 | R1M19 | HC: Y100dS | +++ |
| Ab0407 | R1M20 | HC: Y100dA | +++ |
| Ab0408 | R1M21 | HC: M100eA | +++ |
| Ab0409 | R1M22 | HC: M100eL | +++ |
| Ab0410 | R1M23 | HC: Y100gS | +++ |
| Ab0411 | R1M24 | HC: Y100gA | ++ |
| Ab0412 | R1M25 | HC: M100hA | ++ |
| Ab0413 | R1M26 | HC: M100hL | ++ |
| Ab0414 | R1M27 | HC: F100jA | ++ |

“++++ = Equivalent binding to parental”
“+++ = Slightly weaker binding than parental”

In R2 of engineering, all mutations that had negligible impact on binding were combined. Those mutational combinations that retained binding were then tested for retention time by UPLC-SEC as shown in Table 9 below.

TABLE 9

| | | | LS2A | | |
|---|---|---|---|---|---|
| | | | Ab0223 | SEC | |
| | | | binding | RT | TM |
| Ab | Clone | Mutations | screen | (min) | (° C.) |
| Ab0272 | Clone5 | Parent for this set | ++++ | 16.385 | 75.4 |
| Ab0441 | R2M1 | HC: Y96A + Y100bS | − | | |
| Ab0442 | R2M2 | HC: Y96A + Y100dA | − | | |
| Ab0443 | R2M3 | HC: Y96A + Y100gS | ++ | 16.327 | 77 |
| Ab0444 | R2M4 | HC: Y100bS + Y100dA | − | | |
| Ab0445 | R2M5 | HC: Y100bS + Y100gS | +++ | 14.737 | 78.3 |
| Ab0446 | R2M6 | HC: Y100dA + Y100gS | + | 15.137 | 76.8 |
| Ab0447 | R2M7 | HC: Y53A + Y96A | − | | |
| Ab0448 | R2M8 | HC: Y53A + Y100bS | − | | |
| Ab0449 | R2M9 | HC: Y53A + Y100dA | − | | |
| Ab0450 | R2M10 | HC: Y53A + Y100gS | +++ | 15.787 | 76.6 |
| Ab0451 | R2M11 | HC: Y96A + Y100bS + Y100dA | − | | |
| Ab0452 | R2M12 | HC: Y96A + Y100bS + Y100gS | + | 14.902 | 78.5 |
| Ab0453 | R2M13 | HC: Y96A + Y100dA + Y100gS | − | | |
| Ab0454 | R2M14 | HC: Y100bS + Y100dA + Y100gS | − | | |
| Ab0455 | R2M15 | HC: Y53A + Y96A + Y100bS | − | | |
| Ab0456 | R2M16 | HC: Y53A + Y96A + Y100dA | − | | |
| Ab0457 | R2M17 | HC: Y53A + Y96A + Y100gS | − | | |
| Ab0458 | R2M18 | HC: Y53A + Y100bS + Y100dA | − | | |
| Ab0459 | R2M19 | HC: Y53A + Y100bS + Y100gS | + | 14.662 | 78.1 |
| Ab0460 | R2M20 | HC: Y53A + Y100dA + Y100gS | − | | |
| Ab0461 | R2M21 | HC: Y96A + Y100bS + Y100dA + Y100gS | − | | |
| Ab0462 | R2M22 | HC: Y53A + Y96A + Y100bS + Y100dA | − | | |
| Ab0463 | R2M23 | HC: Y53A + Y96A + Y100bS + Y100gS | − | | |
| Ab0464 | R2M24 | HC: Y53A + Y96A + Y100dA + Y100gS | − | | |
| Ab0465 | R2M25 | HC: Y53A + Y100bS + Y100dA + Y100gS | − | | |
| Ab0466 | R2M26 | HC: Y53A + Y96A + Y100bS + Y100dA + Y100gS | − | | |
| Ab0467 | R2M27 | HC: Y53A + Y54A | − | | |
| Ab0468 | R2M28 | HC: Y95A + Y96A | +++ | 15.995 | 76.4 |
| Ab0469 | R2M29 | HC: Y100aS + Y100bS | − | | |
| Ab0470 | R2M30 | HC: Y100dA + M100eL | − | | |
| Ab0471 | R2M31 | HC: Y100gS + M100hL | ++++ | 16.095 | 75.6 |
| Ab0472 | R2M32 | HC: S30A | ++++ | 23.415 | 73.9 |
| Ab0478 | Trastuzumab | Antibody format matched control | N/A | 14.563 | 80.5 |

“++++ = Equivalent binding to parental”
“+++ = Slightly weaker binding than parental”
“++ = Much weaker binding than parental”
“+ = Very weak binding than parental”
“− = No binding”

Of those antibodies tested, Ab0445 was identified as having the best combination of binding retention time and thermal stability. In R3 of engineering, Ab0445 was used as the parental sequence and mutations were made to remove a putative glycosylation site in CDR H1 and remove potential tryptophan and methionine oxidation liabilities in CDR H3. A single point kinetic screen was performed against LS2A Ab0223+IDV, followed by 5-pt kinetics on a subset. SEC RT and melting temperatures were collected as shown in Tables 10.

TABLE 10

| | | | LS2A | LS2A | | |
|---|---|---|---|---|---|---|
| | | | Ab0223 | Ab0223 | | |
| | | | KD (nM) | KD (nM) | | |
| | | | 1-pt | 5-pt | SEC RT | TM |
| Ab | Clone | Mutations | kinetics | kinetics | (min) | (° C.) |
| Ab0272 | Clone5 | | 24 | 22 | | |
| Ab0445 | R2M5 | Parent for this set | 45 | 27 | 15.02 | 78.2 |
| Ab0595 | R3M1 | HC: S30A | 27 | NT | 17.65 | 77.1 |
| Ab0596 | R3M2 | HC: N28D | 15 | 14 | 16.53 | 77.9 |
| Ab0597 | R3M3 | HC: N28E | 15 | NT | 16.52 | 77.3 |
| Ab0600 | R3M6 | HC: N28T | 15 | NT | 17.32 | 76.7 |
| Ab0601 | R3M7 | HC: S30K | 18 | NT | 17.33 | 77.2 |
| Ab0602 | R3M8 | HC: V29I + S30K | 19 | NT | 17.71 | 78.3 |
| Ab0603 | R3M9 | HC: N28T + V29F | 7 | 7.6 | 17.06 | 77.8 |
| Ab0604 | R3M10 | HC: N28T + V29F + | 18 | NT | 19.55 | 80.2 |
| | | S33A + I34M + H35S | | | | |
| Ab0605 | R3M11 | HC: V29I + S30K + | NB | NT | 18.64 | 76 |
| | | S31D + Y32T + S33Y | | | | |
| Ab0606 | R3M12 | HC: W98L | NB | NT | 15.02 | 78.3 |
| Ab0607 | R3M13 | HC: W98F | poor fit | NT | 15.03 | 79.3 |
| Ab0608 | R3M14 | HC: W98Y | 220 | NT | 14.93 | 78.8 |
| Ab0609 | R3M15 | HC: W98H | 280 | NT | 14.84 | 78.5 |
| Ab0610 | R3M16 | HC: M100eL | 26 | 31 | 15.22 | 78.3 |
| Ab0611 | R3M17 | HC: M100hL | 14 | 22 | 14.96 | 77.3 |
| Ab0612 | R3M18 | HC: M100eL + M100hL | 44 | 31 | 15.20 | 77.5 |
| Ab0613 | R3M19 | HC: Y95S | NB | NT | 15.71 | 81.5 |
| Ab0614 | R3M20 | HC: Y100aS | NB | NT | 14.57 | 79.9 |

In R4a engineering, mutations combining promising findings from R3 were interrogated using Ab0596 as the parental sequence. Combining HC N28D+V29F+M100eL+M100hL (Ab0637) resulted in a molecule with a good kinetic profile and improved SEC RT as shown in Table 11 below.

TABLE 11

| | | | LS2A Ab0223 KD |
|---|---|---|---|
| Ab | Clone | Mutations | (nM) 5-pt kinetics |
| Ab0596 | R3M2 | Parent for this set | 14 |
| Ab0635 | R4M1 | HC: M100hL | NT |
| Ab0636 | R4M2 | HC: V29F + M100hL | NT |
| Ab0637 | R4M3 | HC: V29F + M100eL + | 5.9 |
| | | M100hL | |
| Ab0638 | R4M4 | HC: M100eL + M100hL | NT |

In R4b engineering, mutations were designed on top of the Ab1637 clone R4M3 to further improve hydrophobicity. Mutations were guided by AggScore calculations in the Schrodinger BioLuminate software. Ab698 clone R4M16 showed the most improved affinity and SEC profile as shown in Table 12 below.

TABLE 12

| | | | LS2A Ab0223 KD |
|---|---|---|---|
| Ab | Clone | Mutations | (nM) 3-pt kinetics |
| Ab0637 | R4M3 | Parent for this set | 5.4 |
| Ab0687 | R4M5 | HC: M100eA | 5.5 |
| Ab0688 | R4M6 | HC: Y53N | 29 |
| Ab0689 | R4M7 | HC: Y53D | 42 |
| Ab0690 | R4M8 | HC: Y53H | 11 |
| Ab0691 | R4M9 | HC: Y53K | NB |
| Ab0692 | R4M10 | HC: Y53S | 41 |
| Ab0693 | R4M11 | HC: Y54E | NB |
| Ab0694 | R4M12 | HC: Y54Q | NB |
| Ab0695 | R4M13 | HC: Y54H | NB |
| Ab0696 | R4M14 | HC: Y54K | NB |
| Ab0697 | R4M15 | HC: Y54S | NB |
| Ab0698 | R4M16 | HC: S100bD | 2.9 |
| Ab0699 | R4M17 | HC: S100bK | 33 |
| Ab0700 | R4M18 | HC: L100cA | NB |
| Ab0701 | R4M19 | HC: L100cN | NB |
| Ab0702 | R4M20 | HC: L100cD | NB |
| Ab0703 | R4M21 | HC: L100cH | NB |
| Ab0704 | R4M22 | HC: L100cK | NB |
| Ab0705 | R4M23 | HC: L100cS | NB |
| Ab0706 | R4M24 | HC: Y100dN | 89 |
| Ab0707 | R4M25 | HC: Y100dD | 72 |
| Ab0708 | R4M26 | HC: Y100dH | 37 |
| Ab0709 | R4M27 | HC: Y100dK | 30 |
| Ab0710 | R4M28 | HC: Y100dS | 60 |

TABLE 13

| | | | LS2A | | |
|---|---|---|---|---|---|
| | | | Ab0223 | SEC | |
| | | | KD (nM) | RT | TM |
| Ab | Clone | Mutations | 5-pt kinetics | (min) | (° C.) |
| Ab0637 | R4M3 | Parent for this set | 3.7 | 17.205 | 78.3 |
| Ab0687 | R4M5 | HC: M100eA | 6.4 | 16.188 | 79 |
| Ab0690 | R4M8 | HC: Y53H | 13 | 16.382 | 78.1 |
| Ab0698 | R4M16 | HC: S100bD | 1.7 | 15.095 | 79.1 |
| Ab0901 | R5M1 | HC: Y53H + | 6.6 | 14.813 | 78.9 |
| | | S100bD | | | |
| Ab0902 | R5M2 | HC: S100bD + | 1.6 | 14.798 | 79.4 |
| | | L100eA | | | |
| Ab0903 | R5M3 | HC: Y53H + | 8.1 | 14.548 | 79.1 |
| | | S100bD + | | | |
| | | L100eA | | | |
| Ab0654 | Trastuzumab | | N/A | 14.253 | 81.9 |

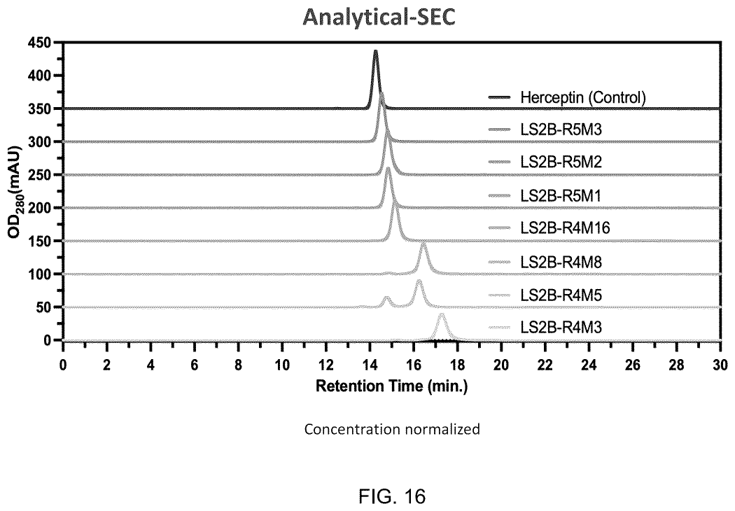

The culmination of these 5 rounds of engineering dramatically improved SEC retention times for LS2BL as observed in UPLC-SEC experiments (FIG. 16).

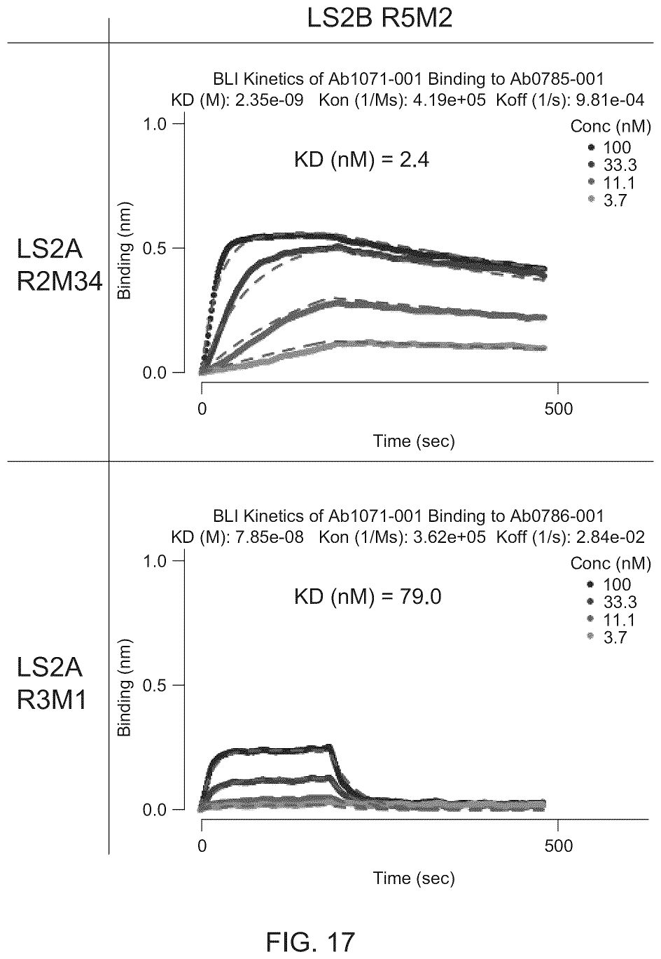

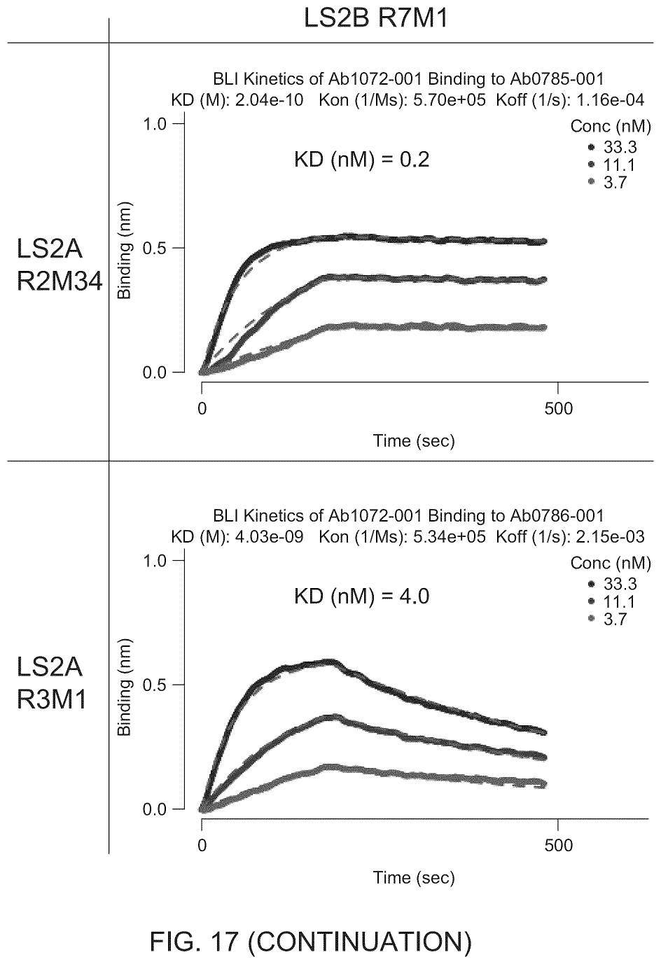

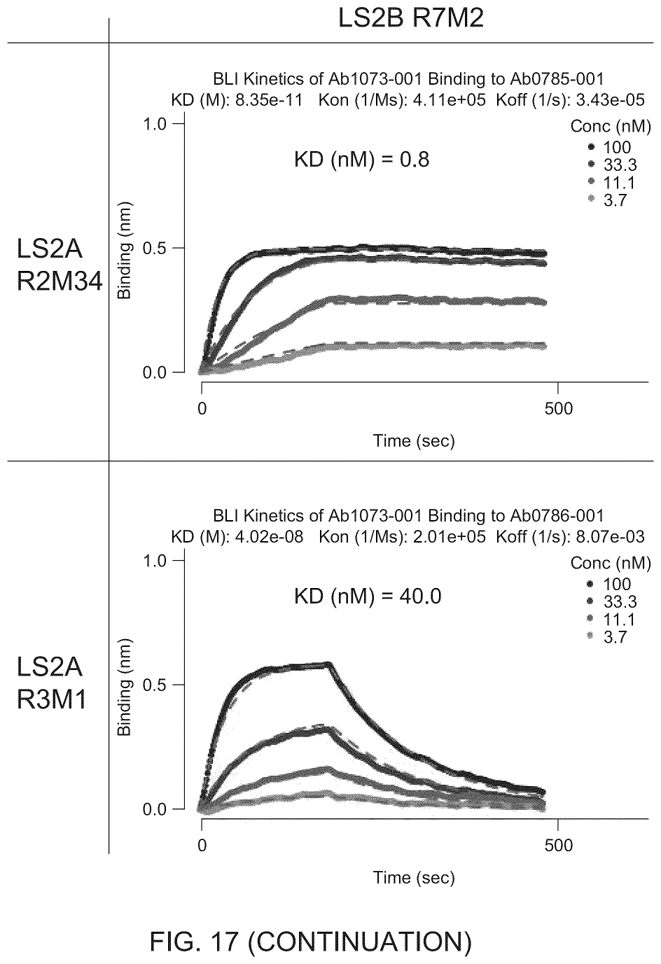

In the parallel optimization of the LS2A component, the preferred LS2A variant R3M1 (described below) showed loss of binding to LS2B3 R5M2 when compared to LS2A R2M34 (FIG. 17), with measured Kds of 2.4 nM and 79 nM respectfully by BLI. In R6 of engineering, alanine scanning of clone R4M16 were performed to find improved binding to LS2A R3M2 (Ab0787) and LS2A R3M1 (Ab0786). Variants were screened for binding to LS2A (Ab0223) and LS2A R3M2 (Ab0787) with a single-point kinetic screen. HC S50A mutation (Ab0936) was found to significantly improve binding affinity to Ab787 as shown in Table 14 below.

TABLE 14

| | | | LS2A | LS2A | LS2A | LS2A |
|---|---|---|---|---|---|---|
| | | | Ab0223 | Ab0787 | Ab0786 | Ab0787 |
| | | | KD (nM) | KD (nM) | KD (nM) | KD (nM) |
| | | | 1-pt | 1-pt | 1-pt | 5-pt |
| Ab | Clone | Mutations | kinetics | kinetics | kinetics | kinetics |
| Ab0698 | R4M16 | Parent for this set | 4 | poor fit | NT | 19 |
| Ab0935 | R6M1 | HC: Y32A | 230 | NB | NT | NT |
| Ab0936 | R6M2 | HC: S50A | <1.0 | 5.7 | NT | 1 |
| Ab0937 | R6M3 | HC: I51A | 2.2 | 42 | NT | NT |
| Ab0938 | R6M4 | HC: S52A | 310 | NB | NT | NT |
| Ab0939 | R6M5 | HC: P52aA | 3.7 | 40 | NT | NT |
| Ab0940 | R6M6 | HC: G55A | 10 | poor fit | NT | NT |
| Ab0941 | R6M7 | HC: S56A | 3.7 | 49 | NT | NT |
| Ab0942 | R6M8 | HC: E97A | 210 | NB | NT | NT |
| Ab0943 | R6M9 | HC: K100A | 130 | NB | NT | NT |
| Ab0944 | R6M10 | HC: D100bA | 7.1 | 100 | NT | NT |
| Ab0945 | R6M11 | HC: L100cA | NB | NB | NT | NT |
| Ab0946 | R6M12 | HC: G100iA | 50 | weak | NT | NT |
| Ab0947 | R6M13 | HC: D101A | 52 | weak | NT | NT |
| Ab0948 | R6M14 | HC: Y102A | 7.3 | poor fit | NT | NT |
| Ab0949 | R6M16 | LC: Q27A | 4.6 | poor fit | NT | NT |
| Ab0950 | R6M17 | LC: Y49A | 19 | poor fit | NT | NT |
| Ab0951 | R6M18 | LC: Y55A | 4 | poor fit | NT | NT |
| Ab0952 | R6M21 | LC: G91A | 6 | poor fit | NT | NT |
| Ab0953 | R6M22 | LC: G92A | 5.6 | 50 | NT | NT |
| Ab0954 | R6M23 | LC: Y93A | 23 | poor fit | NT | NT |
| Ab0955 | R6M24 | LC: S94A | 9.4 | poor fit | NT | NT |
| Ab0956 | R6M25 | LC: L95A | 64 | weak | NT | NT |
| Ab0957 | R6M26 | LC: I96A | 54 | weak | NT | NT |
| Ab0958 | R6M27 | LC: T97A | 4 | 42 | NT | NT |
| Ab0902 | R5M2 | Parent for this set | 5.9 | 33 | weak | NT |
| Ab1034 | R6M28 | HC: Y58A | NT | 16 | 93 | NT |
| Ab1035 | R6M29 | HC: Y95A | NT | weak | NB | NT |
| Ab1036 | R6M30 | HC: G100fS | NT | NB | NB | NT |
| Ab1037 | R6M31 | HC: Y58A + | NT | 38 | weak | NT |
| | | Y95A | | | | |
| Ab1038 | R6M32 | HC: Y58A + | NT | weak | NB | NT |
| | | G100fS | | | | |
| Ab1039 | R6M33 | HC: Y95A + | NT | NB | NB | NT |
| | | G100fS | | | | |
| Ab1040 | R6M34 | HC: Y58A + | NT | NB | NB | NT |
| | | Y95A + G100fS | | | | |
| Ab0903 | R5M3 | Parent for this set | 21 | weak | NB | NT |
| Ab1041 | R6M72 | HC: Y58A + | NT | NB | NB | NT |
| | | Y95A + G100fS | | | | |
| Ab1042 | R6M73 | HC: Y95A + | NT | NB | NB | NT |
| | | G100fS | | | | |
| Ab1043 | R6M74 | HC: Y58A + | NT | weak | NB | NT |
| | | G100fS | | | | |

In R7, combination mutants from R6 were designed to generate further affinity variants. Variants were screened for binding to LS2A Ab0787 and Ab0786 with a single-point kinetic screen. Five-point kinetics was measured for a subset of molecules. Clone LS2B3 R7M 1 and LS2B3 R7M2 demonstrated a K_{D} of 8.6 nM and 40 nM, respectively as shown in Table 15 below and (FIG. 17).

TABLE 15

| | | | LS2A | LS2A | LS2A | LS2A | |
|---|---|---|---|---|---|---|---|
| | | | Ab0787 | Ab0786 | Ab0787 | Ab0786 | |
| | | | KD (nM) | KD (nM) | KD (nM) | KD (nM) | |
| | | | 1-pt | 1-pt | 5-pt | 5-pt | TM |
| Ab | Clone | Mutations | kinetics | kinetics | kinetics | kinetics | (° C.) |
| Ab0902 | R5M2 | Parent for this set | 33 | weak | NT | NT | 77.2 |
| Ab1044 | R7M1 | HC: S50A | 5.1 | 8.2 | 2.3 | 8.6 | 78.3 |
| Ab1045 | R7M2 | HC: S50A + | 18 | 24 | 16 | 40 | 77.7 |
| | | W98Y | | | | | |
| Ab1046 | R7M3 | HC: S50A + | NB | NB | NT | NT | NT |
| | | Y58A + Y95A + | | | | | |
| | | G100fS | | | | | |
| Ab1047 | R7M4 | HC: S50A + | 47 | weak | NT | NT | NT |
| | | Y58A + Y95A | | | | | |
| Ab1048 | R7M5 | HC: S50A + | weak | NB | NT | NT | NT |
| | | Y95A + G100fS | | | | | |
| Ab1049 | R7M6 | HC: S50A + | 31 | NB | NT | NT | NT |
| | | Y58A + G100fS | | | | | |
| Ab1050 | R7M7 | HC: S50A + | NB | NB | NT | NT | NT |
| | | Y58A + Y95A + | | | | | |
| | | W98Y + G100fS | | | | | |
| Ab0903 | R5M3 | Parent for this set | weak | NB | NT | NT | NT |
| Ab1051 | R7M8 | HC: S50A | 18 | 160 | 11 | 55 | NT |
| Ab1052 | R7M9 | HC: S50A + | NB | NB | NT | NT | NT |
| | | Y58A + Y95A + | | | | | |
| | | G100fS | | | | | |
| Ab1053 | R7M10 | HC: S50A + | weak | NB | NT | NT | NT |
| | | Y58A + Y95A | | | | | |
| Ab1054 | R7M11 | HC: S50A + | NB | NB | NT | NT | NT |
| | | Y95A + G100fS | | | | | |
| Ab1055 | R7M12 | HC: S50A + | weak | NB | NT | NT | NT |
| | | Y58A + G100fS | | | | | |
| Ab0654 | Trastuzumab | | N/A | N/A | N/A | N/A | 78.6 |

### LS2A Engineering

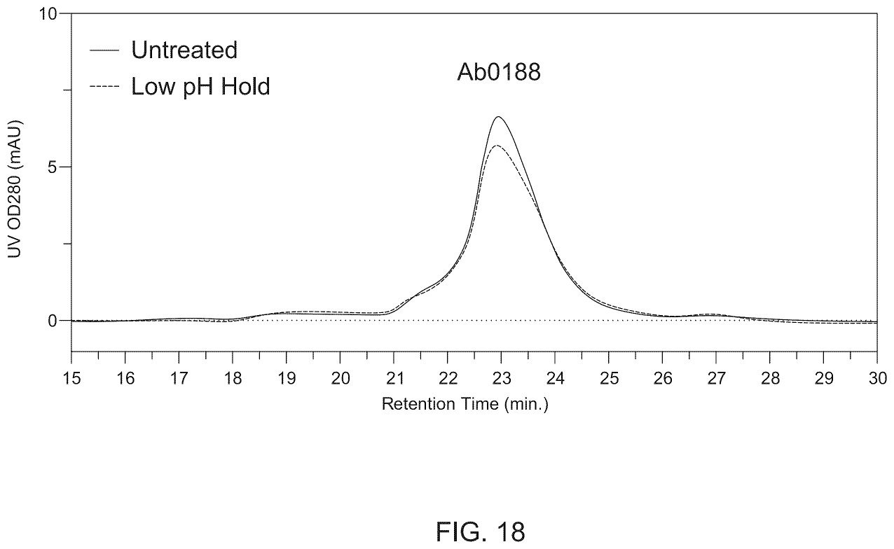

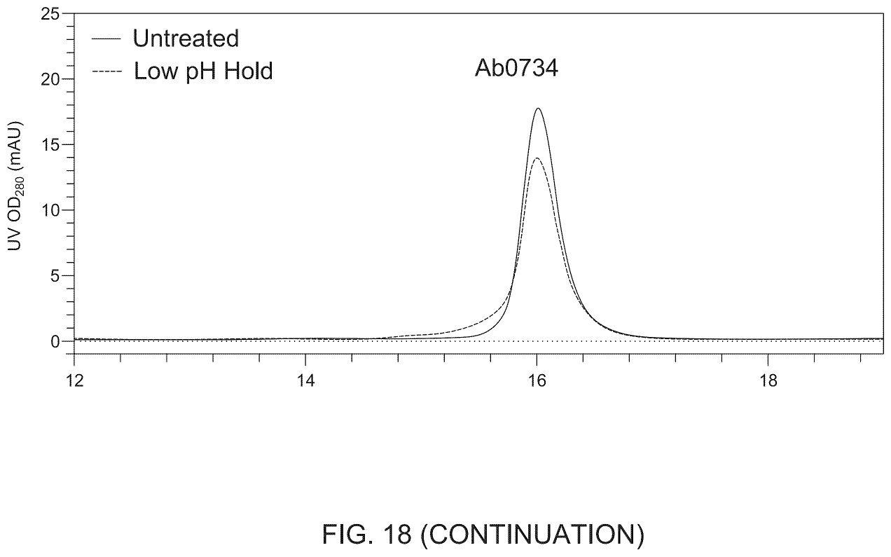

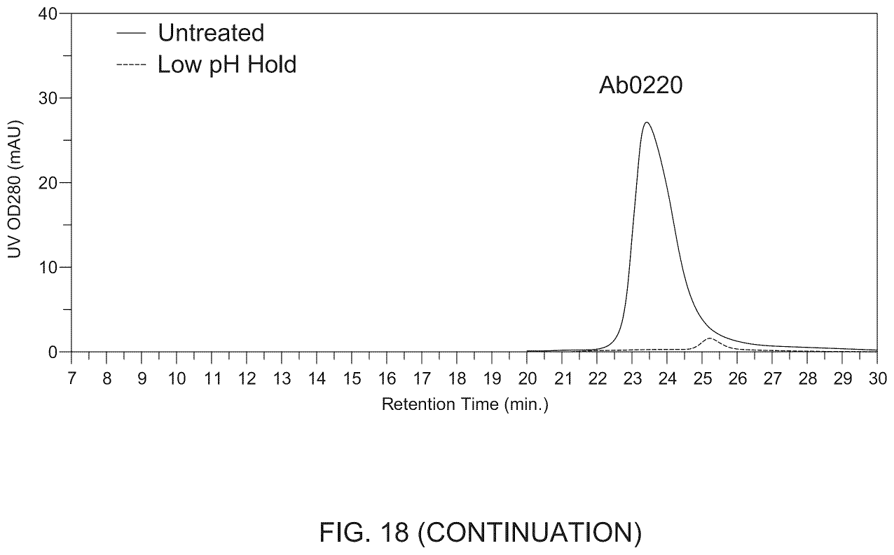

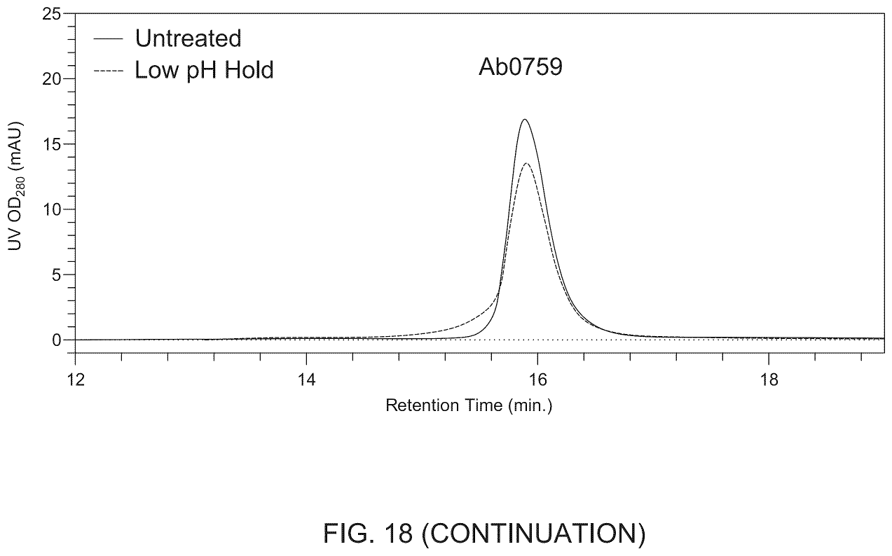

The stability of LS2A scFv constructs was assessed prior and subsequent to initial humanization efforts with a low pH hold UPLC assay and DSF. While the murine parental Ab0188 showed expected resistance to low pH treatment, a significant amount of material was lost with Ab220 (FIG. 18). Ab0188 had a measured Tm of 59.2° C. while Ab0220 had a Tm of 51.4° C. (FIG. 19). These results motivated engineering efforts to improve the stability of humanized LS2A. The first round of engineering sought to improve stability through reformatting of the scFv. A Fab formatted construct was compared to an scFv with VH-VL or VL-VH orientation plus or minus a stabilizing disulfide (HC:G44C+LC:Q100C). Ab0220 remained the most optimal construct based on SEC RT and TM as shown in Table 16 below.

TABLE 16

| | | | SEC | |
|---|---|---|---|---|
| | | | RT | TM |
| Ab | LS2A format | Antibody Format | (min) | (° C.) |
| Ab0188 | Murine VH-VL scFv | monovalent scFv-Fc | 16.043 | 59.2 |
| Ab0220 | Humanized VH-VL scFv | monovalent scFv-Fc | 16.03 | 51.4 |
| Ab0368 | Humanized Fab | monovalent Fab-Fc | 17.523 | 66.1 |
| Ab0615 | Murine Fab | monovalent Fab-Fc | 16.385 | 73.6 |
| Ab0616 | Humanized VL-VH scFv | monovalent scFv-Fc | 17.392 | 52.2 |
| Ab0617 | Humanized VH-VL | monovalent scFv-Fc | 17.064 | 49.2 |
| | scFv + disulfide | | | |
| | (HC: G44C + | | | |
| | LC: Q100C) | | | |
| Ab0618 | Humanized VL-VH | monovalent scFv-Fc | 16.135 | 51.4 |
| | scFv + disulfide | | | |
| | (HC: G44C + | | | |
| | LC: Q100C) | | | |

In R1 of engineering, point mutations were made to remove an oxidation site, deamidation sites, or a hydrophobic residue. In addition, several murine back mutations were tested to improve humanization. Binding kinetics were measured to biotin-IDV and LS2B Ab0637. Ab0663 R1M8 demonstrated the best combination of kinetics and TM as shown in Table 17 below.

TABLE 17

| | | | biotin-IDV KD | LS2B Ab0637 KD | |
|---|---|---|---|---|---|
| | | | (nM) 3-pt | (nM) 3-pt | TM |
| Ab | Clone | Mutations | kinetics | kinetics | (° C.) |
| Ab0220 | | Parent molecule for this set | 69 | 8.9 | 52.4 |
| Ab0617 | | Disulfide (HC: G44C + LC: Q100C) | 24 | 59 | 50.5 |
| Ab0618 | | VL-VH orientation + disulfide | 35 | 33 | 55.2 |
| | | (HC: G44C + LC: Q100C) | | | |
| Ab0656 | R1M1 | HC: W33Y | poor fit | NB | 53.9 |
| Ab0657 | R1M2 | HC: N53S | poor fit | 31 | 53.5 |
| Ab0658 | R1M3 | LC: Y94D | weak | 14 | 53.8 |
| Ab0659 | R1M4 | LC: Y94S | poor fit | 120 | 54 |
| Ab0660 | R1M5 | LC: L95A | 93 | 12 | 55.3 |
| Ab0661 | R1M6 | LC: L95D | 130 | 35 | 54.4 |
| Ab0662 | R1M7 | LC: L95K | 100 | 6.8 | 55 |
| Ab0663 | R1M8 | LC: L95Q | 91 | 9.6 | 55.2 |
| Ab0664 | R1M9 | LC: L95S | poor fit | 16 | 57.6 |
| Ab0665 | R1M10 | LC: F96A | NB | 150 | 59.7 |
| Ab0666 | R1M11 | HC: T73K | 49 | 14 | 52.8 |
| Ab0667 | R1M12 | HC: V78A | weak | 9.8 | 51.7 |
| Ab0668 | R1M13 | LC: L47W | NT | NT | 49.7 |
| Ab0669 | R1M14 | LC: F71Y | poor fit | 12 | 51.3 |

In R2 of engineering, combinations of murine back mutations were made plus and minus disulfide stabilization mutations with an VH-VL to VL-VH orientation swap. A computational residue scanning approach was performed with the Schrodinger BioLuminate software. Several point mutations predicted to improve stability were also selected for evaluation. Kinetics and DSF experiments were performed as previously described. A Low pH hold assay was performed on a subset. Ab0759 R2M34 showed the best overall combination of kinetics, thermostability, and acid stability as shown in Table 18 below and FIG. 18.

TABLE 18

| | | | | | biotin- | LS2B | | |
|---|---|---|---|---|---|---|---|---|
| | | | | Yield | IDV | Ab0637 | | |
| | | | | from | KD (nM) | KD (nM) | | Acid |
| | | scFv | | 50 mL | 3-pt | 3-pt | TM | stability |
| Ab | Clone | Orientation | Mutations | (mg) | kinetics | kinetics | (° C.) | (% MP) |
| Ab0220 | | VH-VL | Humanized scFv (Parent molecule for this set) | | 200 | 5.3 | 52.4 | 33 |
| AB0188 | | VH-VL | Murine LS2A scFv | | 2 | NB | 59.2 | 86 |
| Ab0618 | | VL-VH | Disulfide (HC: G44C + LC: Q100C) | | 160 | 19 | 55.2 | 97 |
| Ab0663 | R1M8 | VH-VL | LC: L95Q | | 77 | 6 | 55.2 | 47 |
| Ab0726 | R2M1 | VL-VH | Disulfide + LC: L95Q | 0.18 | 42 | 15 | 57.8 | 69 |
| Ab0727 | R2M2 | VL-VH | Disulfide + HC: I50V + LC: L95Q | 0.18 | 170 | 150 | 50.0 | NT |
| Ab0728 | R2M3 | VH-VL | LC: S53N + R54L + T56S + L95Q | 0.26 | 73 | 9 | 55.9 | NT |
| Ab0729 | R2M4 | VH-VL | LC: S53N + R54L + T56S + I58V + D60A + | 0.29 | 74 | 9.1 | 55.9 | NT |
| | | | L95Q | | | | | |
| Ab0730 | R2M5 | VH-VL | LC: L47W + S53N + R54L + T56S + I58V + | 0.23 | 41 | 11 | 50.7 | NT |
| | | | D60A + L95Q | | | | | |
| Ab0731 | R2M6 | VL-VH | Disulfide + LC: S53N + R54L + T56S + | 0.24 | 40 | 50 | 60.0 | 84 |
| | | | L95Q | | | | | |
| Ab0732 | R2M7 | VL-VH | Disulfide + LC: S53N + R54L + T56S + | 0.24 | 42 | 90 | 60.2 | NT |
| | | | I58V + D60A + L95Q | | | | | |
| Ab0733 | R2M8 | VL-VH | Disulfide + LC: L47W + S53N + R54L + | 0.15 | 41 | 110 | 59.4 | NT |
| | | | T56S + I58V + D60A + L95Q | | | | | |
| Ab0734 | R2M9 | VH-VL | HC: I50M + S58N + LC: L95Q | 0.30 | 11 | weak | 59.5 | 90 |
| Ab0735 | R2M10 | VH-VL | HC: M48I + I50M + S58N + V67A + M69L + | 0.32 | 9.4 | weak | 59.7 | NT |
| | | | LC: L95Q | | | | | |
| Ab0736 | R2M11 | VL-VH | Disulfide + HC: I50M + S58N + LC: L95Q | 0.27 | 5.4 | weak | 60.7 | 91 |
| Ab0737 | R2M12 | VL-VH | Disulfide + HC: M48I + I50M + S58N + | | NT | NT | NT | NT |
| | | | V67A + M69L + LC: L95Q | | | | | |
| Ab0738 | R2M13 | VH-VL | HC: I50M + S58N + LC: S53N + R54L + | 0.34 | 9 | weak | 59.2 | 68 |
| | | | T56S + L95Q | | | | | |
| Ab0739 | R2M14 | VH-VL | HC: I50M + S58N + LC: S53N + R54L + | 0.30 | 11 | weak | 59.3 | NT |
| | | | T56S + I58V + D60A + L95Q | | | | | |
| Ab0740 | R2M15 | VH-VL | HC: I50M + S58N + LC: L47W + S53N + | 0.22 | 14 | weak | 56.8 | NT |
| | | | R54L + T56S + I58V + D60A + L95Q | | | | | |
| Ab0741 | R2M16 | VH-VL | HC: M48I + I50M + S58N + V67A + M69L + | 0.20 | 8.3 | weak | 59.4 | 98 |
| | | | LC: S53N + R54L + T56S + L95Q | | | | | |
| Ab0742 | R2M17 | VH-VL | HC: M48I + I50M + S58N + V67A + M69L + | 0.21 | 17 | weak | 59.6 | NT |
| | | | LC: S53N + R54L + T56S + I58V + D60A + | | | | | |
| | | | L95Q | | | | | |
| Ab0743 | R2M18 | VH-VL | HC: M48I + I50M + S58N + V67A + M69L + | 0.12 | 19 | NB | 49.3 | NT |
| | | | LC: L47W + S53N + R54L + T56S + I58V + | | | | | |
| | | | D60A + L95Q | | | | | |
| Ab0744 | R2M19 | VL-VH | Disulfide + HC: I50M + S58N + LC: S53N + | 0.13 | 21 | NB | 49.2 | NT |
| | | | R54L + T56S + L95Q | | | | | |
| Ab0745 | R2M20 | VL-VH | Disulfide + HC: I50M + S58N + LC: S53N + | 0.21 | 6.9 | weak | 60.9 | NT |
| | | | R54L + T56S + I58V + D60A + L95Q | | | | | |
| Ab0746 | R2M21 | VL-VH | Disulfide + HC: I50M + S58N + LC: L47W + | 0.12 | 6 | weak | 60.0 | NT |
| | | | S53N + R54L + T56S + I58V + D60A + L95Q | | | | | |
| Ab0747 | R2M22 | VL-VH | Disulfide + HC: M48I + I50M + S58N + | 0.17 | 7.7 | weak | 60.3 | 89 |
| | | | V67A + M69L + LC: S53N + R54L + T56S + | | | | | |
| | | | L95Q | | | | | |
| Ab0748 | R2M23 | VL-VH | Disulfide + HC: M48I + I50M + S58N + | 0.19 | 8.1 | weak | 60.5 | NT |
| | | | V67A + M69L + LC: S53N + R54L + T56S + | | | | | |
| | | | I58V + D60A + L95Q | | | | | |
| Ab0749 | R2M24 | VL-VH | Disulfide + HC: M48I + I50M + S58N + | 0.13 | 9.7 | weak | 59.8 | NT |
| | | | V67A + M69L + LC: L47W + S53N + R54L + | | | | | |
| | | | T56S + I58V + D60A + L95Q | | | | | |
| Ab0750 | R2M25 | VH-VL | HC: M48I + LC: L95Q | 0.29 | 83 | 8 | 55.7 | NT |
| Ab0751 | R2M26 | VH-VL | HC: V67A + M69L + LC: L95Q | 0.16 | 91 | 9.6 | 51.1 | NT |
| Ab0752 | R2M27 | VH-VL | HC: M48I + V67A + M69L + LC: L95Q | | NT | NT | NT | NT |
| Ab0753 | R2M28 | VH-VL | HC: R71V + LC: L95Q | 0.22 | 270 | 12 | 55.4 | NT |
| Ab0754 | R2M29 | VH-VL | HC: R71V + T73K + LC: L95Q | | NT | NT | NT | NT |
| Ab0755 | R2M30 | VH-VL | HC: R71V + T73K + V78A + LC: L95Q | 0.26 | 58 | 10 | 56.1 | NT |
| Ab0756 | R2M31 | VH-VL | HC: M48I + V67A + M69L + R71V + | 0.36 | 290 | 19 | 56.8 | NT |
| | | | T73K + LC: L95Q | | | | | |
| Ab0757 | R2M32 | VH-VL | HC: M48I + V67A + M69L + R71V + | 0.39 | weak | 12 | 57.9 | NT |
| | | | T73K + V78A + LC: L95Q | | | | | |
| Ab0758 | R2M33 | VH-VL | HC: A40R + LC: L95Q | 0.34 | 280 | 5.7 | 57.1 | NT |
| Ab0759 | R2M34 | VH-VL | HC: A40H + LC: L95Q | 0.33 | 140 | 6.1 | 60.4 | 92 |
| Ab0760 | R2M35 | VH-VL | LC: S76H + L95Q | 0.30 | 100 | 9 | 52.7 | NT |
| Ab0761 | R2M36 | VH-VL | LC: A43H + L95Q | 0.29 | 220 | 10 | 51.9 | NT |
| Ab0762 | R2M37 | VH-VL | HC: G44H + LC: L95Q | 0.32 | 110 | 9.1 | 53.1 | NT |
| Ab0763 | R2M38 | VH-VL | HC: G44R + LC: L95Q | 0.27 | 220 | 13 | 52.9 | NT |

In R3 of engineering HC I50M and/or S58N mutations were combined onto clone LS2A R2M34 to improve stability and affinity. Thermostability was improved for Clones R3M1 and R3M2 containing the I50M mutation as shown in Tables 19 and 20 below, and FIG. 19.

TABLE 19

| | | | | LS2B Ab0670 KD | TM |
|---|---|---|---|---|---|
| Ab | Clone | Mutation | Antibody Format | (nM) 5-pt kinetics | (° C.) |
| Ab0785 | R2M34 | Parent for this set | Fab, His-Tag, Avi-Tag | 29 | 70.9 |
| Ab0786 | R3M1 | HC: I50M + S58N | Fab, His-Tag, Avi-Tag | weak | 74.2 |
| Ab0787 | R3M2 | HC: I50M | Fab, His-Tag, Avi-Tag | weak | 74.6 |
| Ab0788 | R3M3 | HC: S58N | Fab, His-Tag, Avi-Tag | 86 | 70.1 |

TABLE 20

| | | | | Free IDV | LS2B Ab1073 | |
|---|---|---|---|---|---|---|
| | | | Antibody | KD (nM) | KD (nM) | TM |
| Ab | Clone | Mutation | Format | 5-pt kinetics | 5-pt kinetics | (° C.) |
| Ab0898 | R2M34 | Parent for this set | scFv-Fc | NT | NT | 52.5 |
| Ab0899 | R3M1 | HC: I50M + S58N | scFv-Fc | 4.6 | 12 | 56.1 |
| Ab0900 | R3M2 | HC: I50M | scFv-Fc | NT | NT | 57.1 |

Affinity of Ab0899 clone R3M 1 to free Indinavir was measured by SPR to be 4.6 nM. Clone R3M1 demonstrated weak binding to Ab0670 LS2B clone R3M2 (TABLE 19), but improved 12 nM K_{D} to Ab1073 LS2B clone R7M2 (TABLE 20 and FIG. 17). In R4 of engineering, mutations were made to weaken the affinity to Indinavir or to improve the thermostability of LS2A. Stability mutations were again guided by in silico residue scanning with the Schrodinger BioLuminate software. Ab1126 R4M32 showed a particularly interesting combination of weakened Indinavir binding with an unaltered LS2B binding and TM relative to the parental molecule Ab 1094 as shown in Table 21 below.

TABLE 21

| | | | | LS2B | |
|---|---|---|---|---|---|
| | | | biotin-IDV | Ab1073 | |
| | | | KD | KD | TM |
| Ab | Clone | Mutations | (nM) | (nM) | (° C.) |
| Ab1094 | R3M1 | Parent | 4.4 | 16 | 58.9 |
| Ab1095 | R4M1 | HC: W33A | 14 | weak | 55.9 |
| Ab1096 | R4M2 | HC: W33F | 15 | NT | 54.8 |
| Ab1097 | R4M3 | HC: W33H | 16 | NT | 54.8 |
| Ab1098 | R4M4 | HC: W33L | 16 | NT | NT |
| Ab1099 | R4M5 | HC: H35A | 140 | NT | NT |
| Ab1100 | R4M6 | HC: H52N | 74 | NB | NT |
| Ab1101 | R4M7 | HC: H52A | 74 | NB | 54.9 |
| Ab1102 | R4M8 | HC: R100A | weak | 20 | 65.4 |
| Ab1103 | R4M9 | HC: N53A | 11 | NT | NT |
| Ab1104 | R4M10 | HC: N53S | 9.1 | 93 | 59.2 |
| Ab1105 | R4M11 | HC: N53Q | 11 | NT | 59.6 |
| Ab1106 | R4M12 | HC: S54A | 6.5 | NT | 54.1 |
| Ab1107 | R4M13 | HC: I51A | weak | NT | NT |
| Ab1108 | R4M14 | HC: G55A | 11 | NT | NT |
| Ab1109 | R4M15 | HC: G56S | 6.3 | NT | NT |
| Ab1110 | R4M16 | HC: T57A | 4.9 | NT | NT |
| Ab1111 | R4M17 | HC: G95A | 27 | 19 | 56.9 |
| Ab1112 | R4M18 | HC: D96A | poor fit | NT | NT |
| Ab1113 | R4M19 | HC: Y97A | 66 | NB | NT |
| Ab1114 | R4M20 | HC: V98A | 50 | weak | 59.9 |
| Ab1115 | R4M21 | HC: S99A | 19 | NT | NT |
| Ab1116 | R4M22 | HC: D101A | NB | NB | NT |
| Ab1117 | R4M23 | HC: Y102A | 57 | 13 | 57.5 |
| Ab1118 | R4M24 | HC: S31A | 20 | NT | NT |
| Ab1119 | R4M25 | HC: Y32A | 120 | NB | NT |
| Ab1120 | R4M26 | HC: K12Q | 34 | NT | NT |
| Ab1121 | R4M27 | HC: K23I | 43 | 11 | 54.7 |
| Ab1122 | R4M28 | HC: D101L | NB | NB | NT |
| Ab1123 | R4M29 | HC: T68Q | 13 | NT | NT |
| Ab1124 | R4M30 | LC: Y91A | 22 | NT | NT |
| Ab1125 | R4M31 | LC: Y91F | 44 | poor fit | 58 |
| Ab1126 | R4M32 | LC: Y94A | 64 | 26 | 59.1 |
| Ab1127 | R4M33 | LC: F96L | NB | NT | NT |
| Ab1128 | R4M34 | LC: S92A | 37 | NT | NT |
| Ab1129 | R4M35 | LC: G93A | 30 | NT | NT |
| Ab1130 | R4M36 | LC: T97A | 36 | NT | NT |
| Ab1131 | R4M37 | LC: H34A | 12 | NT | NT |
| Ab1132 | R4M38 | LC: Y32A | 17 | NT | NT |

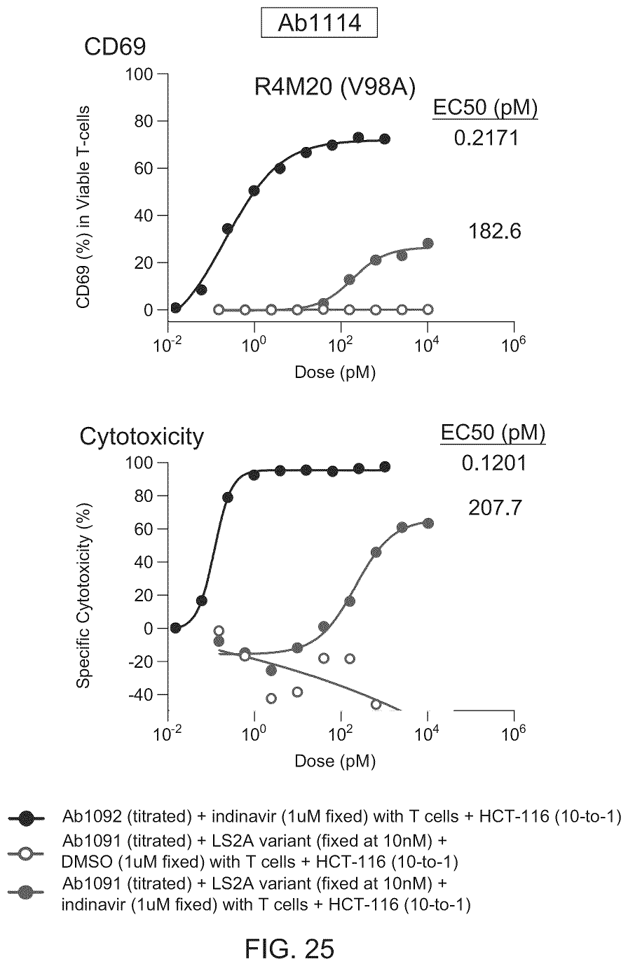

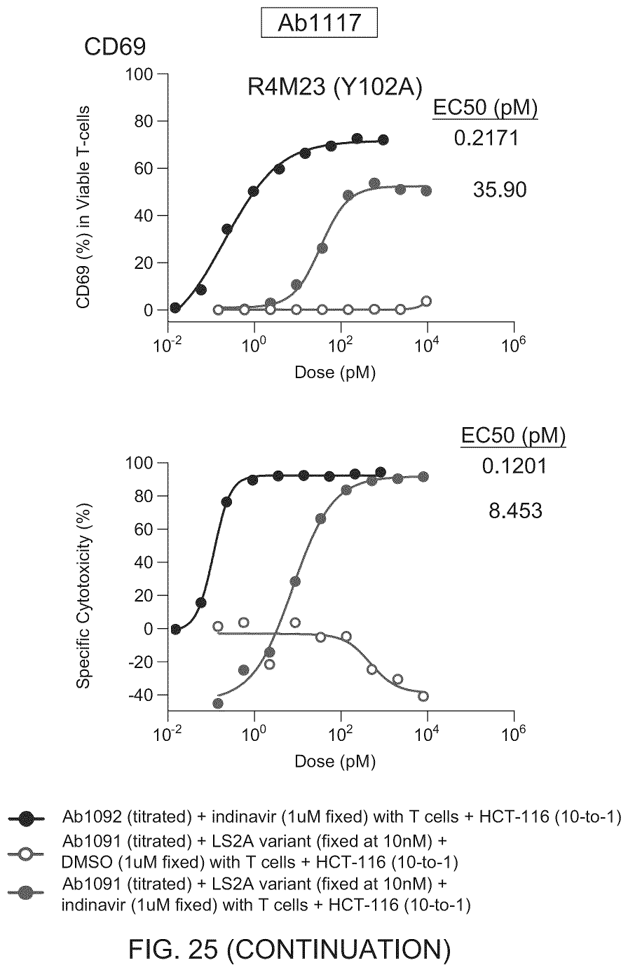

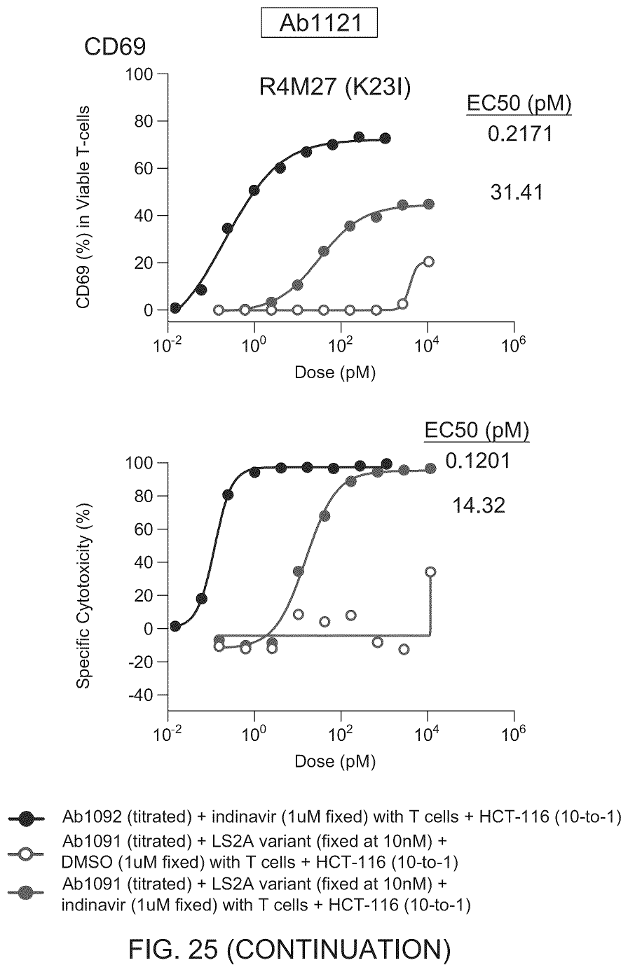

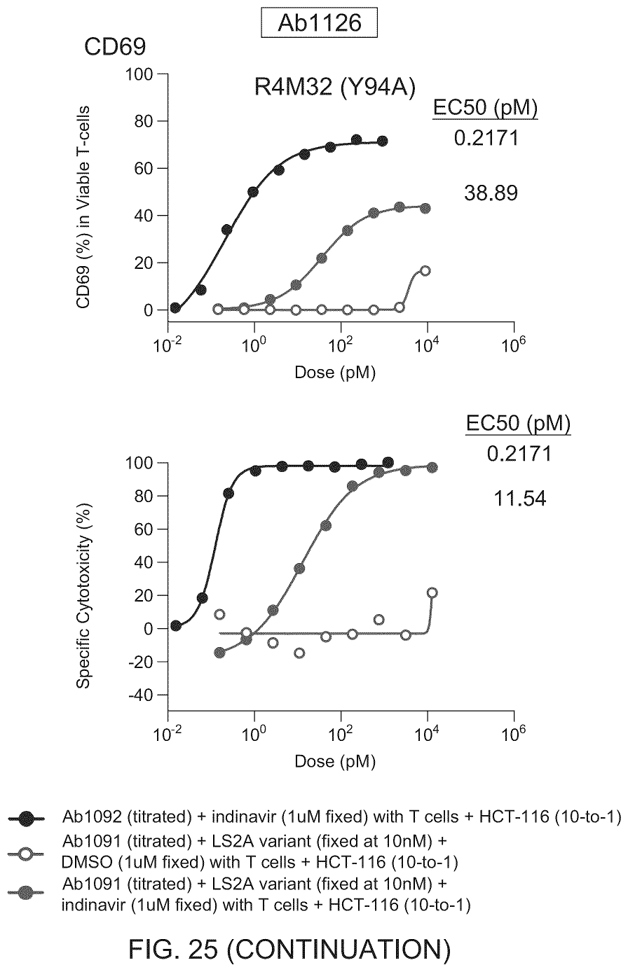

Ab1126 R4M32 also showed comparable T-cell activation and T-cell mediated cellular cytotoxicity in an in vitro co-culture assay (FIG. 25).

### LITE Switch Characterization

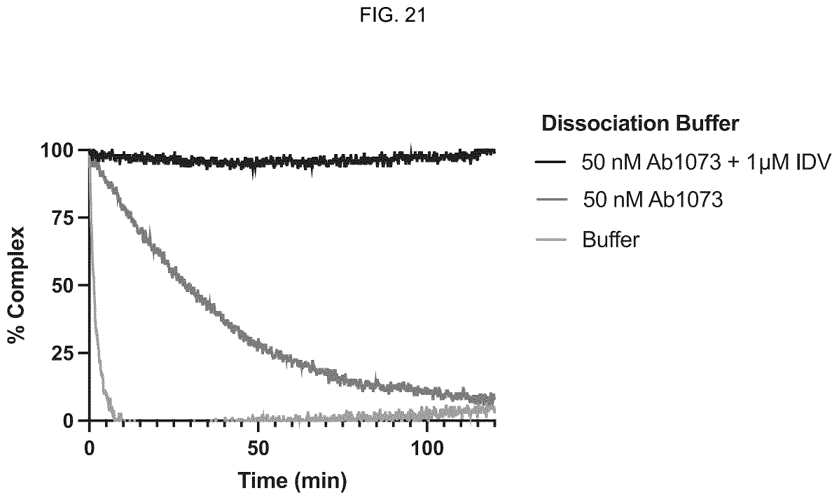

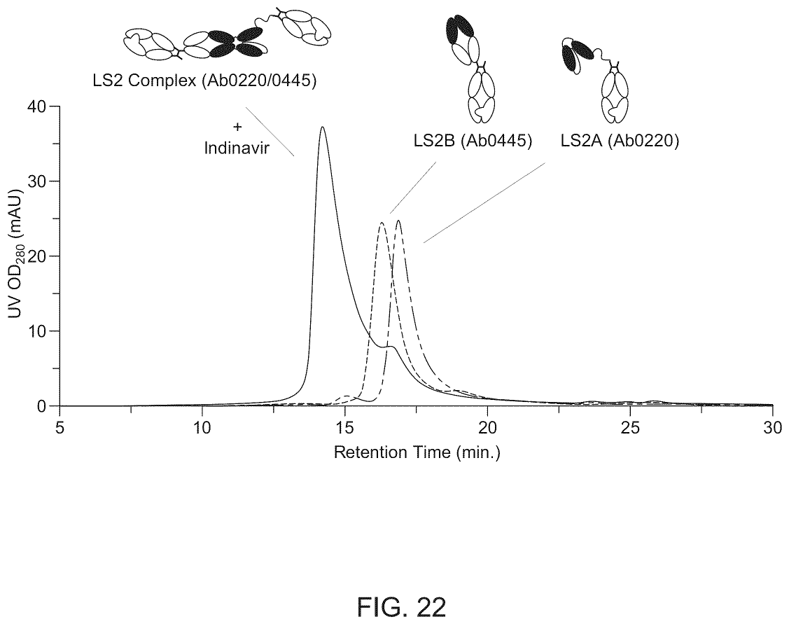

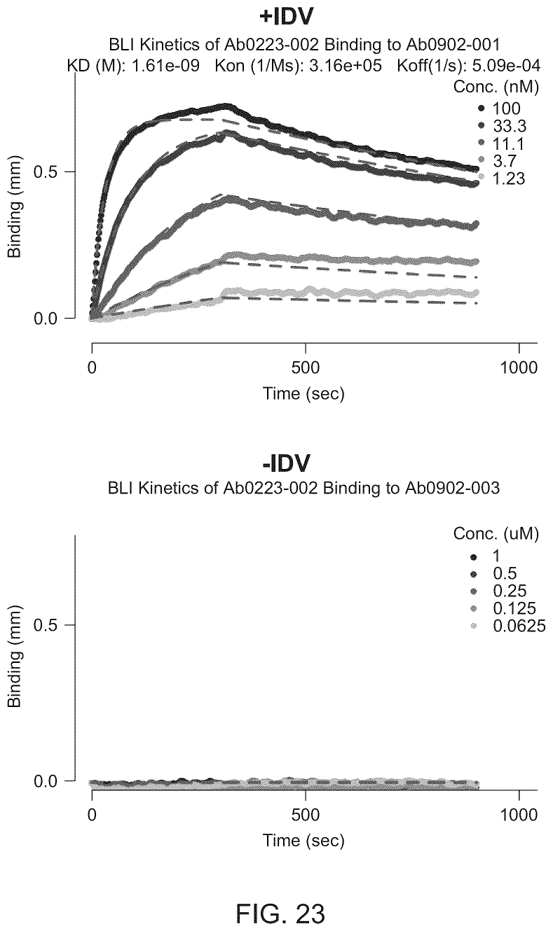

A series of biophysical experiments was conducted to further elucidate a mechanism for Indinavir switch assembly. Biolayer interferometry experiments were performed to measure LS2A and LS21B assembly in the presence and absence of saturating concentrations of Indinavir. Ab0223 demonstrated an affinity of 1.6 nM for binding to Ab0902 in the presence of 10 μM IDV but showed no detectable interaction in the absence of Indinavir (FIG. 23). To estimate the size and relative stoichiometries of the assembled LS2A/Indinavir/LS2B complex, Ab0220 and Ab0445 were analyzed separately or in combination by UPLC-SEC using a MabPAC 4.6×150 mm column pre-equilibrated with 1×PBS-HCl at pH 7.0 and 10 μM Indinavir. Relative to the proteins analyzed separately, the combination of Ab0220/Ab0445 eluted at an earlier retention time consistent with a heterodimeric complex (FIG. 22). A BLI kinetics assay was performed to assess the reversibility kinetics of the LS2A/LS2B complex upon Indinavir washout. Data showed that when concentrations of Ab1073 and IDV remained constant, no LITE Switch reversibility was observed. In 2 hours of IDV washout, the LITE Switch dissociated to nearly 0% complex, whereas complete complex dissociation was observed in 10 min upon removal of both Ab1073 and IDV (FIG. 21).

### SP34 Engineering

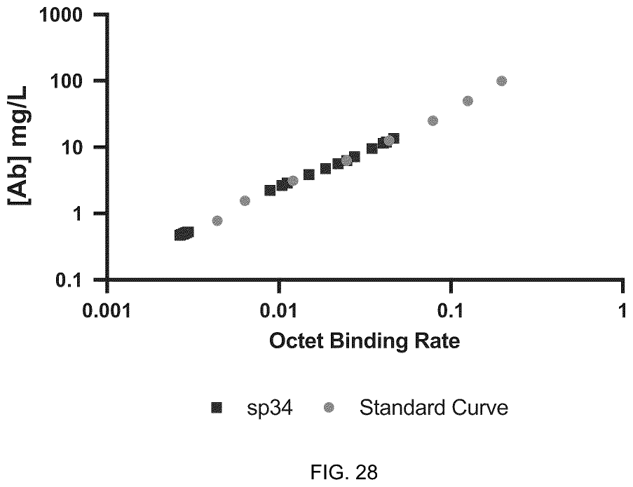

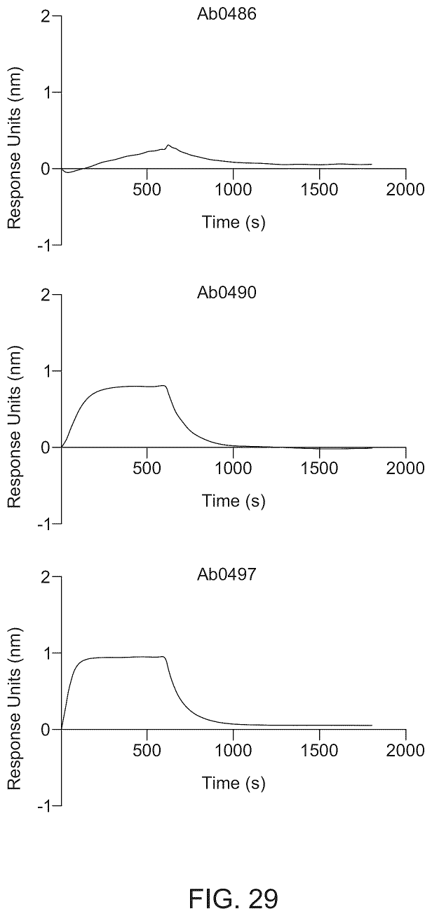

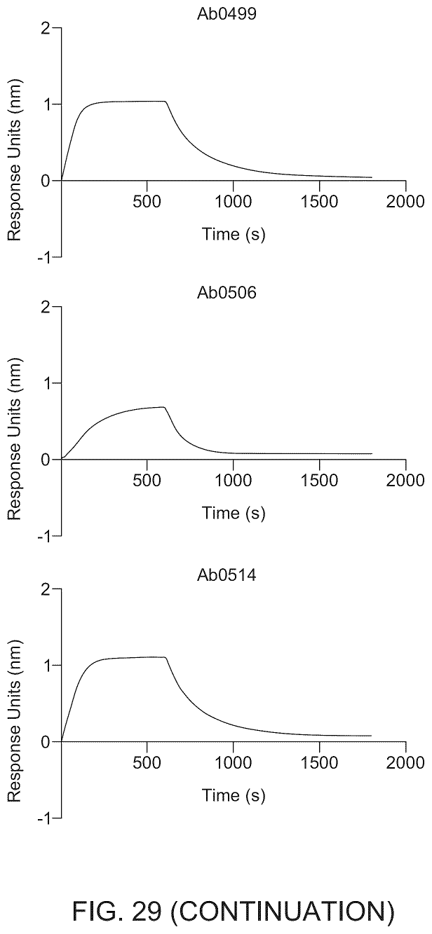

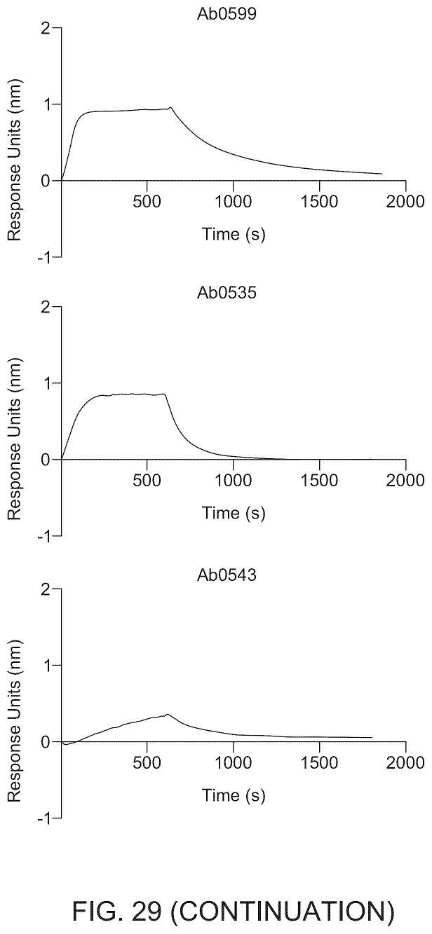

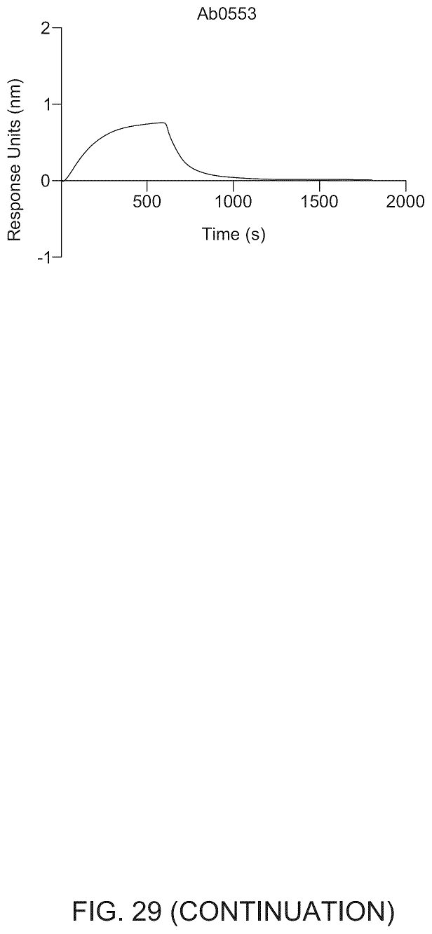

The previously described murine CD3 binding clone, SP34, was humanized. SP34 was humanized by grafting CDR sequences onto various human frameworks including IGHV3-73*01, IGHV3-23*03, IGHV7-4-1*04, IGHV1-46*02, IGLV7-43*01, IGLV8-61*01, IGLV1-51*01, IGLV2-8*01, IGKV3D-11*03, IGKV1-6*01, and IGKV4-1*01. Back mutations on these frameworks were also explored. 73 designs were first tested for expression in Expi293F cells using a BLI supernatant quantitation assay. 18 out of the 73 designs showed detectable expression levels (FIG. 28). 9 of those 18 designs showed detectable binding to CD3E CD3e ECD by BLI (FIG. 29). Five-point kinetics to the human (Ag0052) and cyno (Ag0052) CD3e N-terminal peptide AA1-26 fused to a human IgG1 Fc were measured for a subset of the variants as shown in Table 22 below.

TABLE 22

| | | huCD3e | huCD3e | cyCD3e |
|---|---|---|---|---|
| | | ECD BLI | peptide-Fc | peptide-Fc |
| | | binding | Ag0052 KD (nM) | Ag0052 KD (nM) |
| Ab | Clone | screen | 5-pt kinetics | 5-pt kinetics |
| Ab0486 | hSP34v1 | Yes | NT | NT |
| Ab0487 | hSP34v2 | No | NT | NT |
| Ab0488 | hSP34v3 | No | NT | NT |
| Ab0489 | hSP34v4 | No | NT | NT |
| Ab0490 | hSP34v5 | Yes | 330 | 320 |
| Ab0491 | hSP34v6 | No | NT | NT |
| Ab0492 | hSP34v7 | No | NT | NT |
| Ab0493 | hSP34v8 | No | NT | NT |
| Ab0494 | hSP34v9 | No | NT | NT |
| Ab0495 | hSP34v10 | No | NT | NT |
| Ab0496 | hSP34v11 | No | NT | NT |
| Ab0497 | hSP34v12 | Yes | 180 | 170 |
| Ab0498 | hSP34v13 | No | NT | NT |
| Ab0499 | hSP34v14 | Yes | 53 | 50 |
| Ab0500 | hSP34v15 | No | NT | NT |
| Ab0501 | hSP34v16 | No | NT | NT |
| Ab0502 | hSP34v17 | No | NT | NT |
| Ab0503 | hSP34v18 | No | NT | NT |
| Ab0504 | hSP34v19 | No | NT | NT |
| Ab0505 | hSP34v20 | No | NT | NT |
| Ab0506 | hSP34v21 | Yes | 420 | 540 |
| Ab0507 | hSP34v22 | No | NT | NT |
| Ab0508 | hSP34v23 | No | NT | NT |
| Ab0509 | hSP34v24 | No | NT | NT |
| Ab0510 | hSP34v25 | No | NT | NT |
| Ab0511 | hSP34v26 | No | NT | NT |
| Ab0512 | hSP34v27 | No | NT | NT |
| Ab0513 | hSP34v28 | No | NT | NT |
| Ab0514 | hSP34v29 | Yes | 55 | 50 |
| Ab0515 | hSP34v30 | No | NT | NT |
| Ab0516 | hSP34v31 | No | NT | NT |
| Ab0517 | hSP34v32 | No | NT | NT |
| Ab0518 | hSP34v33 | No | NT | NT |
| Ab0519 | hSP34v34 | No | NT | NT |
| Ab0520 | hSP34v35 | No | NT | NT |
| Ab0521 | hSP34v36 | No | NT | NT |
| Ab0522 | hSP34v37 | No | NT | NT |
| Ab0523 | hSP34v38 | No | NT | NT |
| Ab0524 | hSP34v39 | No | NT | NT |
| Ab0525 | hSP34v40 | No | NT | NT |
| Ab0526 | hSP34v41 | No | NT | NT |
| Ab0527 | hSP34v42 | No | NT | NT |
| Ab0528 | hSP34v43 | No | NT | NT |
| Ab0529 | hSP34v44 | No | NT | NT |
| Ab0530 | hSP34v45 | No | NT | NT |
| Ab0531 | hSP34v46 | No | NT | NT |
| Ab0532 | hSP34v47 | No | NT | NT |
| Ab0533 | hSP34v48 | No | NT | NT |
| Ab0534 | hSP34v49 | No | NT | NT |
| Ab0535 | hSP34v50 | Yes | 230 | 240 |
| Ab0536 | hSP34v51 | No | NT | NT |
| Ab0537 | hSP34v52 | No | NT | NT |
| Ab0538 | hSP34v53 | No | NT | NT |
| Ab0539 | hSP34v54 | No | NT | NT |
| Ab0540 | hSP34v55 | No | NT | NT |
| Ab0541 | hSP34v56 | No | NT | NT |
| Ab0542 | hSP34v57 | No | NT | NT |
| Ab0543 | hSP34v58 | Yes | 560 | 270 |
| Ab0544 | hSP34v59 | No | NT | NT |
| Ab0545 | hSP34v60 | No | NT | NT |
| Ab0546 | hSP34v61 | No | NT | NT |
| Ab0547 | hSP34v62 | No | NT | NT |
| Ab0548 | hSP34v63 | No | NT | NT |
| Ab0549 | hSP34v64 | No | NT | NT |
| Ab0550 | hSP34v65 | No | NT | NT |
| Ab0551 | hSP34v66 | No | NT | NT |
| Ab0552 | hSP34v67 | No | NT | NT |
| Ab0553 | hSP34v68 | Yes | 200 | 180 |
| Ab0554 | hSP34v69 | No | NT | NT |
| Ab0555 | hSP34v70 | No | NT | NT |
| Ab0556 | hSP34v71 | No | NT | NT |
| Ab0557 | hSP34v72 | No | NT | NT |
| Ab0558 | hSP34v73 | No | NT | NT |
| Ab0559 | murine | Yes | 18 | 21 |
| | SP34 | | | |

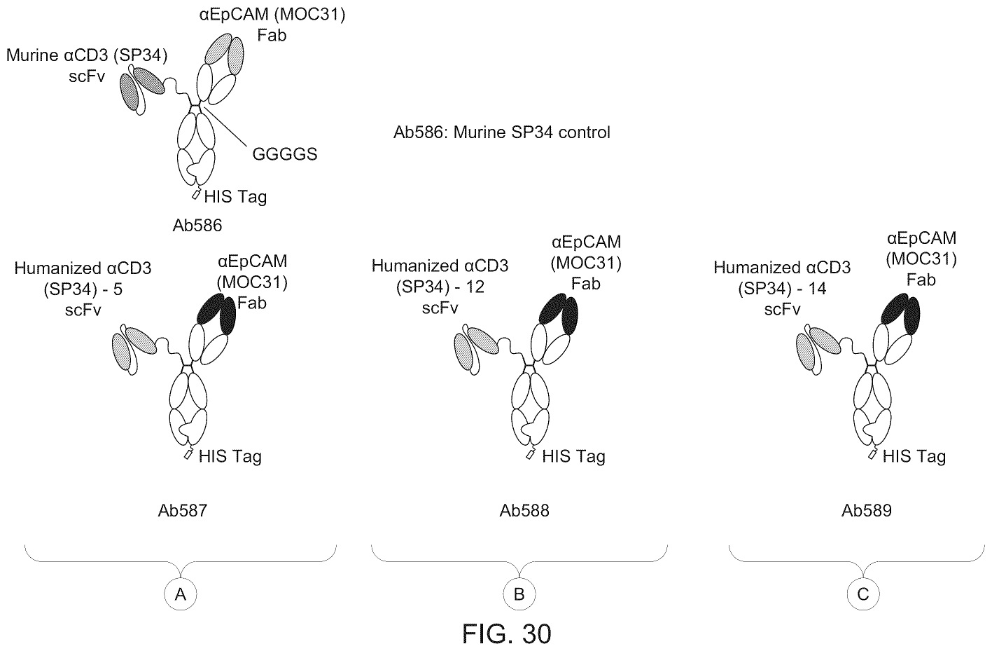

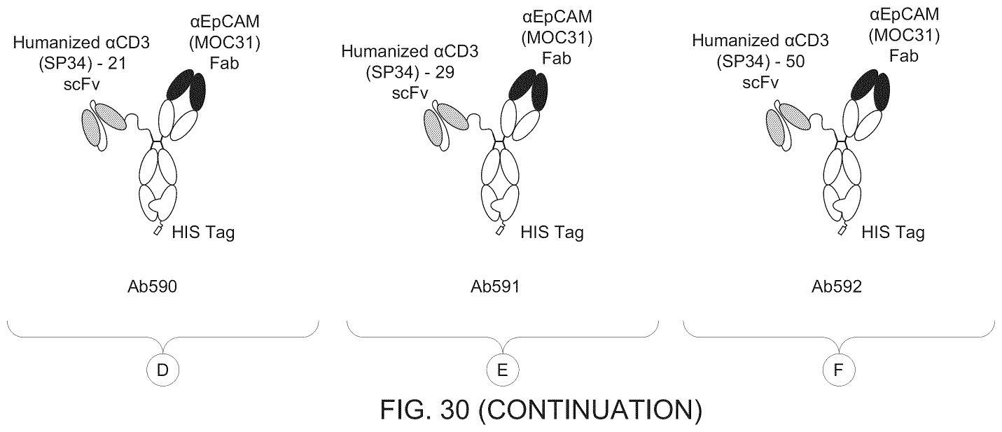

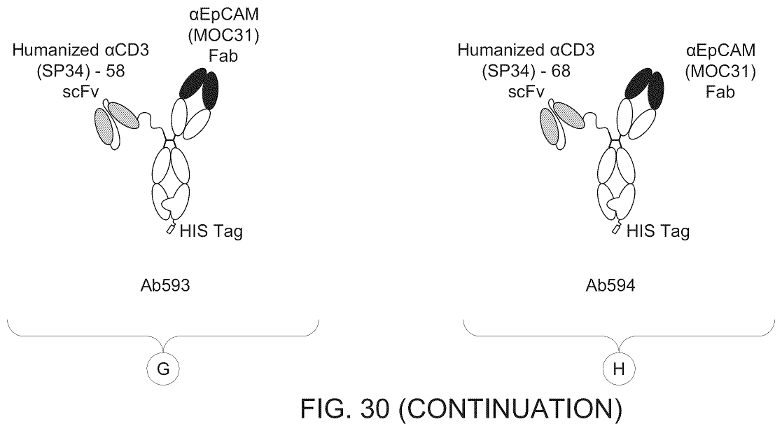

A subset of these confirmed binders were re-formatted into an EpCAM T-cell Engager format and tested for T-cell activation and T-cell mediated cellular cytotoxicity in an in vitro co-culture assay. All variants were tested in a T-cell dependent cellular cytotoxicity (TDCC) co-culture assay to measure the ability to redirect T cells to kill target cells, and the ability to activate T cells. MCF-7, a human breast cancer cell line, endogenously expresses high levels of EpCAM (Casaletto et al 2019; doi: 10.1073/pnas. 1819085116), was used as the target cells. All SP34 variants showed potent T-cell activation and TDCC at low pM concentrations (FIG. 30).

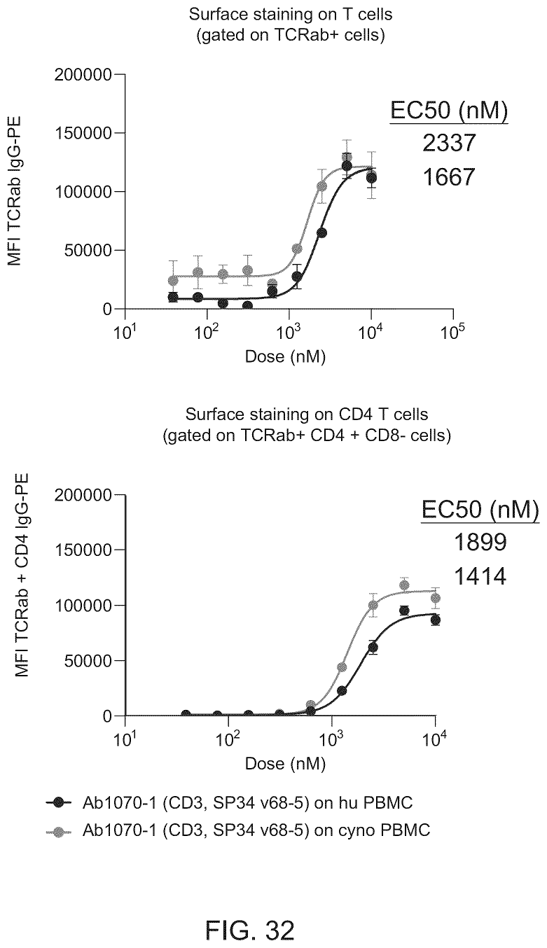

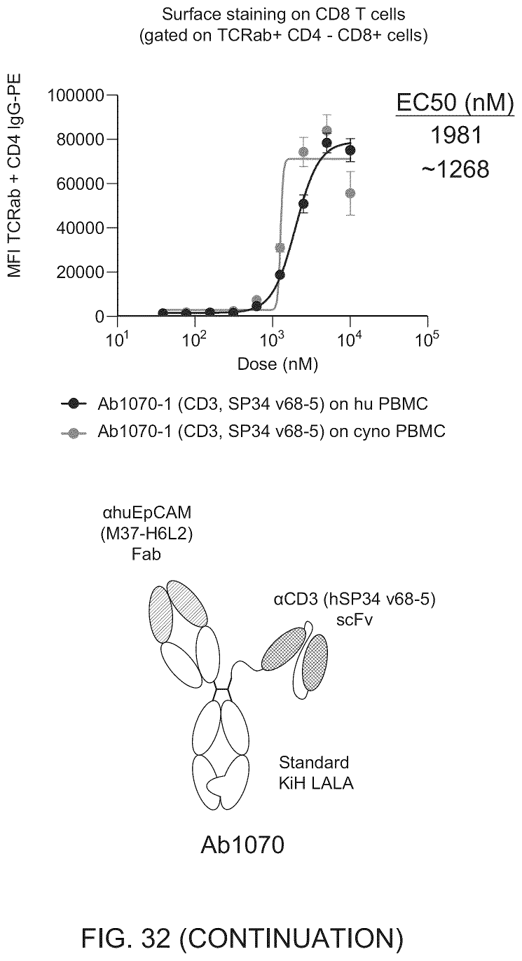

The binding of the preferred clone hSP34v68 was assessed for binding to human and cyno PBMCs by flow-cytometry-based cell-surface staining. Detectable and saturable binding to human and cyno TCRab+ cells, TCRab+ CD4+ CD8− cells, and TCRab+ CD34− CD8+ cells was observed (FIG. 32). Pharmacokinetic properties of Ab640 hSP34v68 in mice were also assessed and compared to an isotype matched control with the Ab632 Herceptin clone. hSP34v68 at a dose of 6.7 mg/kg demonstrated a half-life of 112 h and a CL of 18.6 mL/kg/day (FIG. 31).

Using hSP34v68 as a starting point for R1 of engineering, mutations were designed to improve the humanization, modulate the affinity, and remove potential liabilities in clone hSP34v68. Affinity to the human CD3e N-terminal peptide AA1-26 fused to a human IgG1 Fc (Ag0060) was measured by SPR and thermostability was measured for a subset by DSF as shown in Table 23 below.

TABLE 23

| | | | huCD3e peptide-Fc | |
|---|---|---|---|---|
| | | | Ag0060 KD (nM) | TM |
| Ab | Clone | Mutation | 5-pt kinetics | (° C.) |
| Ab0640 | v68 | Parent | 250 | 60.7 |
| Ab0769 | v68-1 | HC: A49G | NT | 61.7 |
| Ab0770 | v68-2 | HC: G96K | NT | 61.0 |
| Ab0771 | v68-3 | LC: P8delete | NT | 55.8 |
| Ab0772 | v68-4 | LC: L54R | 250 | 62.4 |
| Ab0773 | v68-5 | LC: S56P | 85 | 63.4 |
| Ab0774 | v68-6 | LC: L54R + S56P | 98 | 63.9 |
| Ab0775 | v68-7 | HC: A49G + LC: L54R + S56P | 63 | 64.1 |
| Ab0776 | v68-8 | HC: G96K + LC: L54R + S56P | 110 | 63.8 |
| Ab0907 | v68-9 | HC: N30S + LC: L54R + S56P | 230 | NT |
| Ab0908 | v68-10 | HC: T31A + LC: L54R + S56P | NT | NT |
| Ab0909 | v68-11 | HC: Y32A + LC: L54R + S56P | NB | NT |
| Ab0910 | v68-12 | HC: R52S + LC: L54R + S56P | NB | NT |
| Ab0911 | v68-13 | HC: N53S + LC: L54R + S56P | 480 | NT |
| Ab0912 | v68-14 | HC: N53Q + LC: L54R + S56P | 660 | NT |
| Ab0913 | v68-15 | HC: N54A + LC: L54R + S56P | 130 | NT |
| Ab0914 | v68-16 | HC: N54Q + LC: L54R + S56P | 140 | NT |
| Ab0915 | v68-17 | HC: N100S + LC: L54R + S56P | 250 | NT |
| Ab0916 | v68-18 | HC: N100Q + LC: L54R + S56P | 240 | NT |
| Ab0917 | v68-19 | HC: S100aA + LC: L54R + S56P | 1300 | NT |
| Ab0918 | v68-20 | HC: Y100bA + LC: L54R + S56P | 220 | NT |
| Ab0919 | v68-21 | HC: V100cA + LC: L54R + S56P | 860 | NT |
| Ab0920 | v68-22 | HC: S100dA + LC: L54R + S56P | 2700 | NT |
| Ab0921 | v68-23 | HC: W100eY + LC: L54R + S56P | 100 | NT |
| Ab0922 | v68-24 | LC: T29A + L54R + S56P | NT | NT |
| Ab0923 | v68-25 | LC: S30A + L54R + S56P | NT | NT |
| Ab0924 | v68-26 | LC: Y32A + L54R + S56P | Weak | NT |
| Ab0925 | v68-27 | LC: N52S + L54R + S56P | 100 | NT |
| Ab0926 | v68-28 | LC: K52S + L54R + S56P | 200 | NT |
| Ab0927 | v68-29 | LC: L54R + S56P + W91Y | 2200 | NT |
| Ab0928 | v68-30 | LC: L54R + S56P + S93A | 180 | NT |
| Ab0929 | v68-31 | LC: L54R + S56P + N94A | 240 | NT |
| Ab0930 | v68-32 | LC: L54R + S56P + W96Y | NT | NT |

In R2 of engineering, mutations were combined to modulate the affinity and remove potential deamidation sites. Kinetics for binding CD3E of the variants was measured by SPR. Thermostability was also measured as shown in Table 24 below.

TABLE 24

| | | | huCD3e peptide-Fc | |
|---|---|---|---|---|
| | | | Ag0060 KD (nM) 5- | TM |
| Ab | Clone | Mutation | pt kinetics | (° C.) |
| Ab1091 | v68-5 | Parent | 190 | 62.2 |
| Ab1133 | v68-33 | HC: N54A | 190 | 62.1 |
| Ab1134 | v68-34 | HC: N30S | 300 | 63.7 |
| Ab1135 | v68-35 | HC: N53S | 520 | 61.5 |
| Ab1136 | v68-36 | HC: N53Q | 610 | 62.5 |
| Ab1137 | v68-37 | HC: S100aA | 660 | 63 |

### EpCAM Engineering

Through a hybridoma campaign where mice were immunized with an EpCAM ECD Fc-fusion, the murine EpCAM binding antibody clone M37 was produced. Clone M37 was humanized by grafting CDR sequences onto human consensus VH3 and VK1 frameworks. Various combinations of back mutations (HC: S49A N73D N76S L78V A93T R94L and LC: I2N L47W F71Y) were tested to find optimal binding kinetics and stability. Of these designs, clone hM37-H6L2 (Ab0835) demonstrated the highest affinity to

human (Ag0065;
QEECVCENYKLAVNCFVNNNRQCQCTSVGAQNTVICSKLAAKCLVMKAE
MNGSKLGRRAKPEGALQNNDGLYDPDCDESGLFKAKQCNGTSTCWCVNT
AGVRRTDKDTEITCSERVRTYWIIIELKHKAREKPYDSKSLRTALQKEI
TTRYQLDPKFITSILYENNVITIDLVQNSSQKTQNDVDIADVAYYFEKD
VKGESLFHSKKMDLTVNGEQLDLDPGQTLIYYVDEKAPEFSMQGLKHHH
HHH; SEQ ID NO: 3838)
and
cyno (Ag0066;
QKECVCENYKLAVNCFLNDNGQCQCTSIGAQNTVLCSKLAAKCLVMKAE
MNGSKLGRRAKPEGALQNNDGLYDPDCDESGLFKAKQCNGTSTCWCVNT
AGVRRTDKDTEITCSERVRTYWIIIELKHKAREKPYDVQSLRTALEEAI
KTRYQLDPKFITNILYEDNVITIDLVQNSSQKTQNDVDIADVAYYFEKD
VKGESLFHSKKMDLRVNGEQLDLDPGQTLIYYVDEKAPEFSMQGLKHHH
HHH; SEQ ID NO: 3839)

EpCAM ECD as shown in Table 25 below.

TABLE 25

| | | huEpCAM | cyEpCAM | huEpCAM | cyEpCAM | |
|---|---|---|---|---|---|---|
| | | ECD | ECD | ECD | ECD | |
| | | Ag0065 | Ag0066 | Ag0065 | Ag0066 | |
| | | KD (nM) | KD (nM) | KD (nM) | KD (nM) | |
| | | 5-pt | 5-pt | 5-pt | 5-pt | |
| | | single- | single- | multi- | multi- | |
| | | cycle | cycle | cycle | cycle | TM |
| Ab | Clone | kinetics | kinetics | kinetics | kinetics | (° C.) |
| Ab0588 | MOC31 | 3.5 | NB | NT | NT | NT |
| Ab0682 | M37 | 5.7 | 7.3 | NT | NT | NT |
| Ab0823 | M37 | NT | NT | 11.0 | 7.6 | 68.5 |
| Ab0824 | hM37-H1L1 | Weak | Weak | NT | NT | NT |
| Ab0825 | hM37-H2L1 | 6.4 | 6.9 | NT | NT | NT |
| Ab0826 | hM37-H3L1 | Weak | Weak | NT | NT | NT |
| Ab0827 | hM37-H4L1 | 11.4 | 6.0 | NT | NT | NT |
| Ab0828 | hM37-H5L1 | 4.8 | 2.5 | NT | NT | NT |
| Ab0829 | hM37-H6L1 | NT | NT | NT | NT | NT |
| Ab0830 | hM37-H1L2 | Weak | Weak | NT | NT | NT |
| Ab0831 | hM37-H2L2 | 6.4 | 6.1 | NT | NT | NT |
| Ab0832 | hM37-H3L2 | Weak | Weak | NT | NT | NT |
| Ab0833 | hM37-H4L2 | 6.4 | 6.3 | NT | NT | NT |
| Ab0834 | hM37-H5L2 | 4.4 | 2.2 | NT | NT | NT |
| Ab0835 | hM37-H6L2 | 2.7 | 1.5 | 3.0 | 2.2 | 74.0 |
| Ab0836 | hM37-H1L3 | Weak | Weak | NT | NT | NT |
| Ab0837 | hM37-H2L3 | 6.6 | 7.5 | NT | NT | NT |
| Ab0838 | hM37-H3L3 | Weak | Weak | NT | NT | NT |
| Ab0839 | hM37-H4L3 | 6.7 | 8.8 | NT | NT | NT |
| Ab0840 | hM37-H5L3 | 5.5 | 3.8 | NT | NT | NT |
| Ab0841 | hM37-H6L3 | 4.1 | 2.5 | NT | NT | NT |
| Ab0842 | hM37-H1L4 | Weak | Weak | NT | NT | NT |
| Ab0843 | hM37-H2L4 | 9.9 | 8.7 | NT | NT | NT |
| Ab0844 | hM37-H3L4 | Weak | Weak | NT | NT | NT |
| Ab0845 | hM37-H4L4 | 8.9 | 8.8 | NT | NT | NT |
| Ab0846 | hM37-H5L4 | 6.2 | 6.3 | NT | NT | NT |
| Ab0847 | hM37-H6L4 | 5.7 | 4.3 | NT | NT | NT |

Once M37 was successfully humanized, additional engineering was conducted to further improve the construct. In R1 of engineering, mutations were designed to remove potential oxidation or isomerization liabilities. Stability mutants were also included based on in silico residue scanning results in the Schrodinger BioLuminate software. The affinity of hM37-H6L2 variants to Ag0065 and Ag0066 were measured by SPR and thermostability was measured for a subset by DSF as shown in Table 26 below.

TABLE 26

| | | | huEpCAM | cyEpCAM | huEpCAM | cyEpCAM | |
|---|---|---|---|---|---|---|---|
| | | | ECD | ECD | ECD | ECD | |
| | | | Ag0065 | Ag0066 | Ag0065 | Ag0066 | |
| | | | KD (nM) | KD (nM) | KD (nM) | KD (nM) | |
| | | | 5-pt | 5-pt | 5-pt | 5-pt | |
| | | | single- | single- | multi- | multi- | |
| | | | cycle | cycle | cycle | cycle | TM |
| Ab | Clone | Mutation | kinetics | kinetics | kinetics | kinetics | (° C.) |
| Ab0835 | hM37-H6L2 | Parent for | 3.0 | poor fit | 2.3 | 1.7 | 70.2 |
| | | this set | | | | | |
| Ab1008 | hM37-H6L2v1 | HC: D73N | 11.0 | 8.0 | NT | NT | NT |
| Ab1009 | hM37-H6L2v2 | HC: S76N | 3.1 | 2.2 | 2.3 | 1.4 | 70.7 |
| Ab1010 | hM37-H6L2v3 | HC: T93A | 8.5 | 2.0 | NT | NT | NT |
| Ab1011 | hM37-H6L2v4 | HC: L94R | poor fit | 9.9 | NT | NT | NT |
| Ab1012 | hM37-H6L2v5 | HC: N31S | 9.7 | 6.2 | NT | NT | NT |
| Ab1013 | hM37-H6L2v6 | HC: W33A | poor fit | poor fit | NT | NT | NT |
| Ab1014 | hM37-H6L2v7 | HC: W33F | NT | NT | NT | NT | NT |
| Ab1015 | hM37-H6L2v8 | HC: W33H | poor fit | poor fit | NT | NT | NT |
| Ab1016 | hM37-H6L2v9 | HC: W33Y | 7.2 | 7.7 | NT | NT | NT |
| Ab1017 | hM37-H6L2v10 | HC: N35S | 7.6 | 4.5 | NT | NT | NT |
| Ab1018 | hM37-H6L2v11 | HC: D96E | 3.0 | 2.1 | 2.8 | 1.6 | 69.9 |
| Ab1019 | hM37-H6L2v12 | HC: G97A | 2.5 | 2.0 | 1.8 | 1.1 | NT |
| Ab1020 | hM37-H6L2v13 | HC: N35H | 9.1 | 17.0 | NT | NT | 68 |
| Ab1021 | hM37-H6L2v14 | HC: A40S | 3.1 | 2.3 | NT | NT | NT |
| Ab1022 | hM37-H6L2v15 | HC: A40H | 2.8 | 2.2 | NT | NT | 69.6 |
| Ab1023 | hM37-H6L2v16 | HC: S52cY | 2.0 | 0.9 | NT | NT | NT |
| Ab1024 | hM37-H6L2v17 | HC: A60V | 2.3 | 2.0 | 1.6 | 0.94 | 73.2 |
| Ab1025 | hM37-H6L2v18 | HC: S102Y | 2.2 | 2.5 | NT | NT | NT |
| Ab1026 | hM37-H6L2v19 | LC: W91A | 1.7 | 1.4 | 0.93 | 0.95 | 69.1 |
| Ab1027 | hM37-H6L2v20 | LC: W91F | 9.1 | 10.0 | NT | NT | NT |
| Ab1028 | hM37-H6L2v21 | LC: W91H | 8.8 | 6.7 | NT | NT | NT |
| Ab1029 | hM37-H6L2v22 | LC: W91Y | 7.1 | 4.1 | NT | NT | NT |
| Ab1030 | hM37-H6L2v23 | LC: A43S | 3.3 | 3.1 | NT | NT | NT |
| Ab1031 | hM37-H6L2v24 | LC: S76Q | 2.8 | 2.2 | NT | NT | NT |
| Ab1065 | hM37-H6L2v25 | HC: D96E + | NT | NT | poor fit | poor fit | 72.3 |
| | | LC: W91A | | | | | |
| Ab1066 | hM37-H6L2v26 | HC: G97A + | NT | NT | 5.7 | 3 | 70.4 |
| | | LC: W91A | | | | | |

In R2 of engineering, combinations of mutations were generated based on improved variants from R1. The affinities to Ag0065 and Ag0066 were measured by SPR and thermostability was measured by DSF as shown in Table 27 below.

TABLE 27

| | | | huEpCAM ECD | cyEpCAM ECD | |
|---|---|---|---|---|---|
| | | | Ag0065 KD (nM) 5- | Ag0066 KD (nM) | |
| | | | pt multi-cycle | 5-pt multi-cycle | TM |
| Ab | Clone | Mutation | kinetics | kinetics | (° C.) |
| Ab1069 | hM37-H6L2 | Parent for this set | 3.6 | 1.4 | 72.0 |
| Ab1088 | hM37-H6L2v12 | HC: G97A | 2.7 | 1.1 | 70.1 |
| Ab1089 | hM37-H6L2v28 | HC: A60V + G97A | 2.2 | 1.2 | 71 |
| Ab1090 | hM37-H6L2v29 | HC: A60V + | 2.1 | 1.2 | 75.7 |
| | | LC: W91A | | | |

### Specificity of the M37 Clone in TDCC Assays

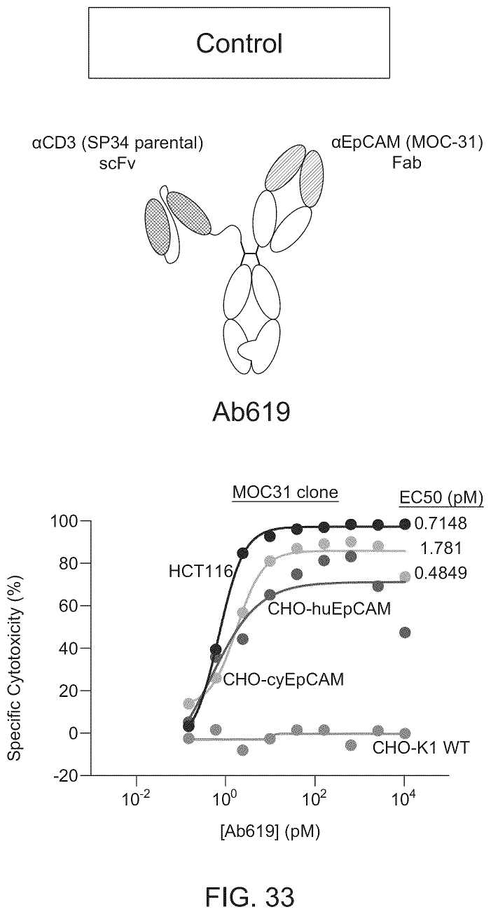

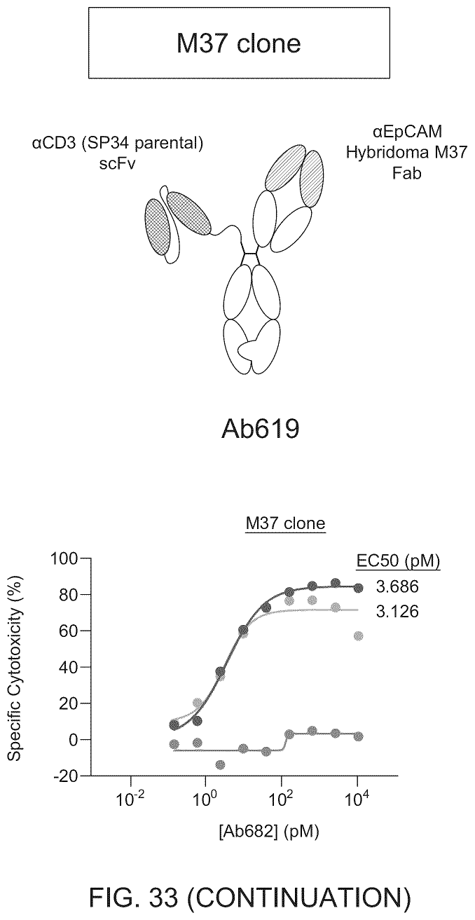

The M37 clone was expressed as a conventional T cell engager (TCE) with anti-CD3 and anti-EpCAM binding domains on the same heterodimeric Fc domain (Ab682). A historical anti-EpCAM clone, MOC31, was used as a benchmark and cloned into another TCE (Ab619). This TCE format was used to assess the potency, specificity and species reactivity of M37 for in a T cell mediated cellular cytotoxicity (TDCC) co-culture assay (FIG. 33). Four different target cell lines were used for the assay. HCT-116, a human colorectal cancer cell line, endogenously expresses high levels of EpCAM (Casaletto et al 2019; doi: 10.1073/pnas. 1819085116). Chinese hamster ovary (CHO-K1) cells do not express EpCAM and the wild-type (WT) cells were used as a negative control (CHO-K1 WT). Two CHO-derived cell lines were transduced with expression vectors driving the expression of either human EpCAM (CHO-huEpCAM) or cynomolgus macaque EpCAM (CHO-cyEpCAM). The MOC31 clone induced TDCC of HCT-116, CHO-huEpCAM and CHO-cyEpCAM target cells, but not CHO-K1 WT. The M37 clone induced TDCC of huEpCAM and CHO-cyEpCAM target cells, but not CHO-K1 WT (HCT-116 was not tested with this clone in this experiment). Altogether, this data demonstrates that human or cynomolgus macaque EpCAM expression is required for target cell killing with MOC31 or M37.

### EpCAM Expression is Required for Binding of the M37 Clone as Measured by Flow Cytometry

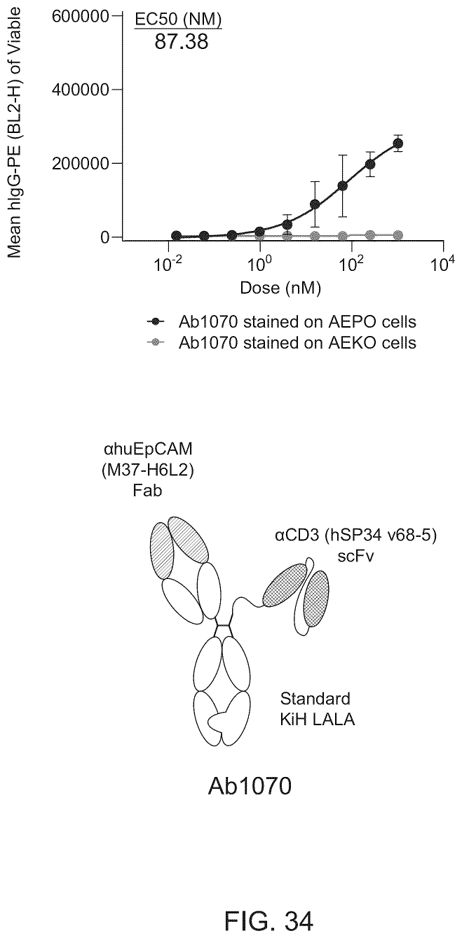

Surface binding was measured on an isogenic panel of EpCAM-knockout and EpCAM-overexpressing cell lines derived from A-431, a human epidermoid carcinoma cell line (FIG. 34). AEKO and AEPO are isogenic control cell lines in which there is zero EpCAM expression (AEKO) or high EpCAM expression (AEKO-E4). AEKO is a commercially available subclone of A-431 in which human EpCAM expression is “knocked out” (disrupted) by a frameshift mutation (catalog number ab261902; Abcam, Cambridge, UK). AEPO was developed using the AEKO cell line as a starting point and restoring expression of human EpCAM by transducing with a lentiviral vector containing human EpCAM downstream of an EF1α promoter. Cells were stained with Ab1070, a conventional TCE containing the M37-H6L2 variant of the M37 clone, at a range of concentrations from 0.015 nM-1 μM. Surface binding was observed on the AEKO-E4 cells but not the AEKO cells, therefore demonstrating that EpCAM expression is both necessary and sufficient for binding of the M37 clone to target cells.

### EpCAM Expression is Required for TDCC Mediated by the M37 Clone

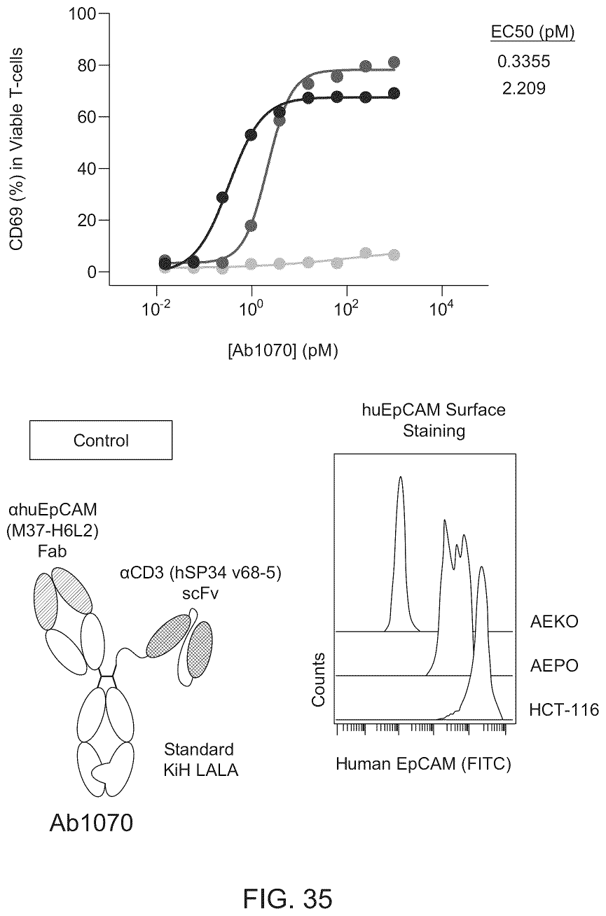

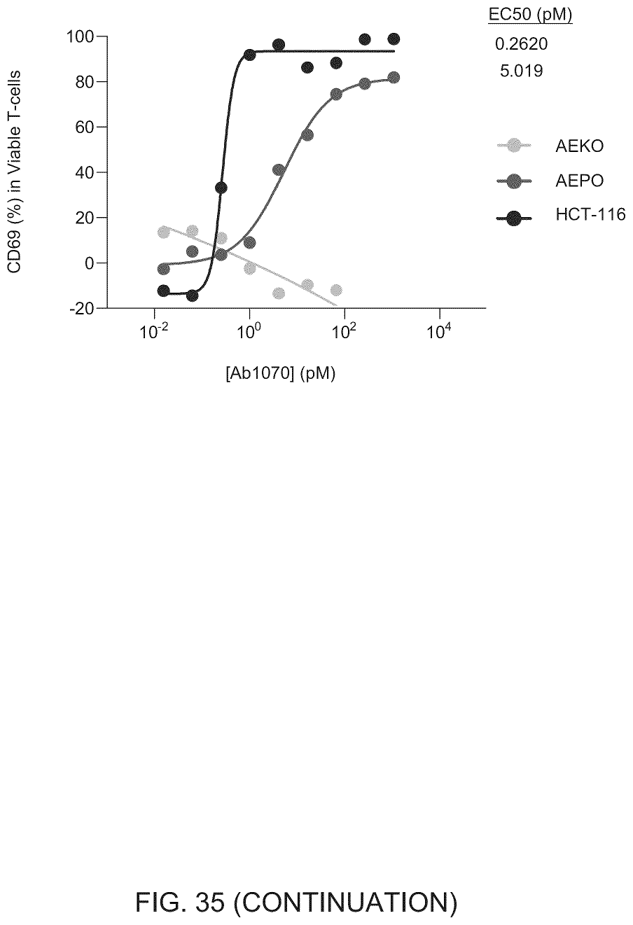

The HCT-116, AEKO and AEPO cell lines described above were used as target cells in a TDCC co-culture assay with Ab1070 (FIG. 35). Only the HCT-116 and AEPO cell lines, which express EpCAM, were killed by TDCC or induced activation of T-cells (as measured by CD69 upregulation). The AEKO cell line, which lacks EpCAM, was spared from TDCC and did not induce T-cell activation. Taken together, this data indicates that EpCAM expression on a target cell is both necessary and sufficient to activate T-cells and redirect them to kill the target cell. EpCAM structure-activity relationship (SAR) campaign

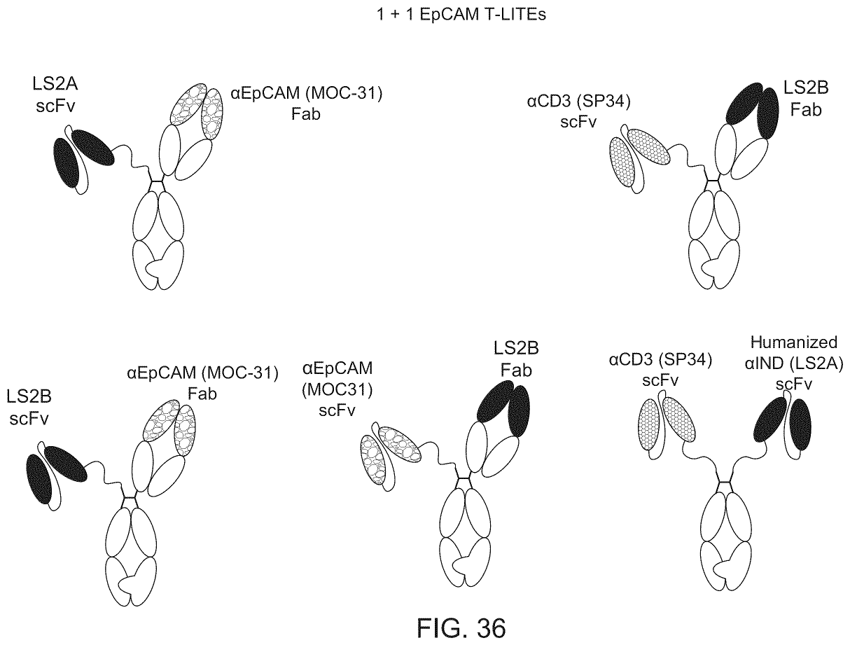

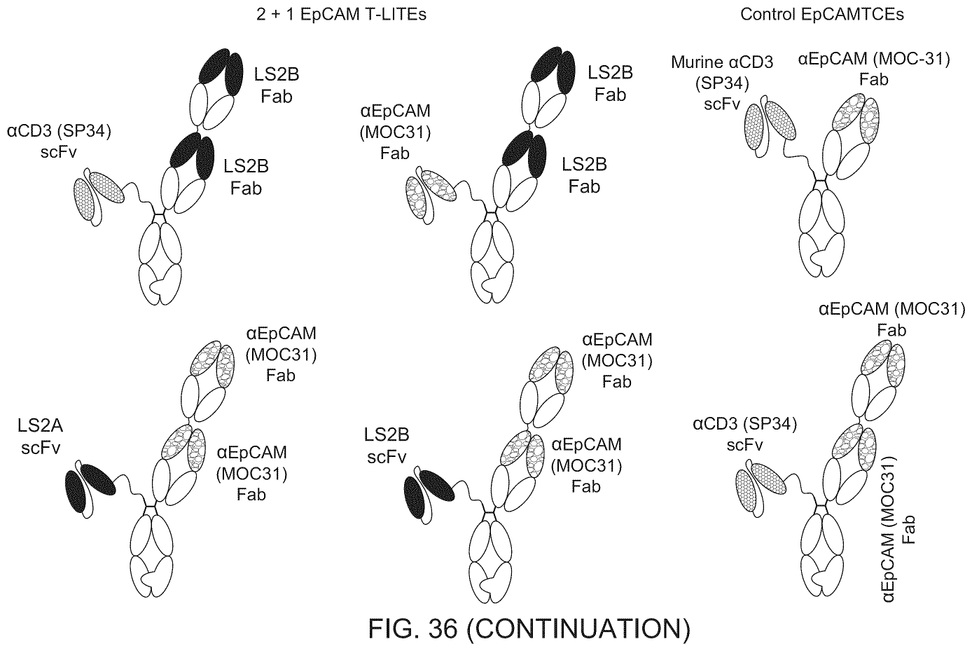

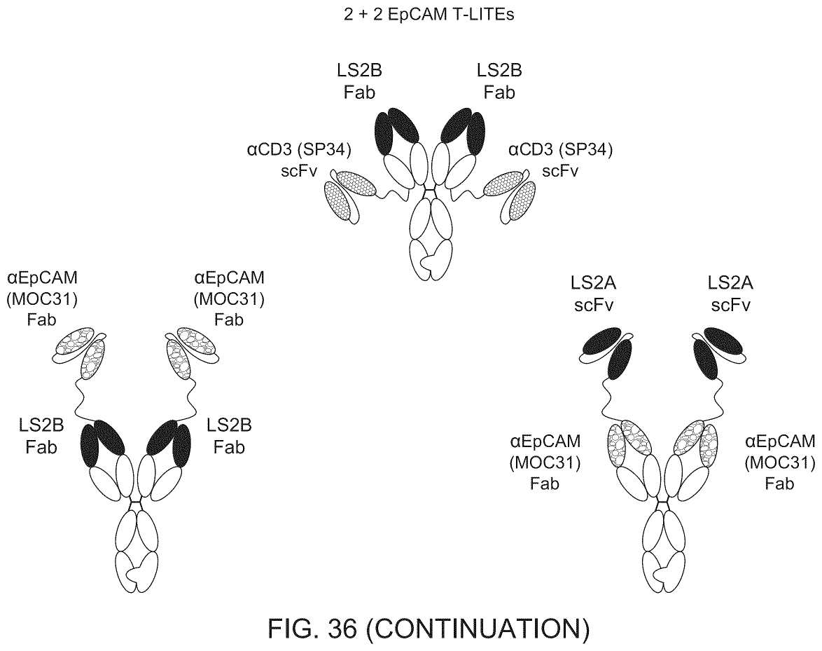

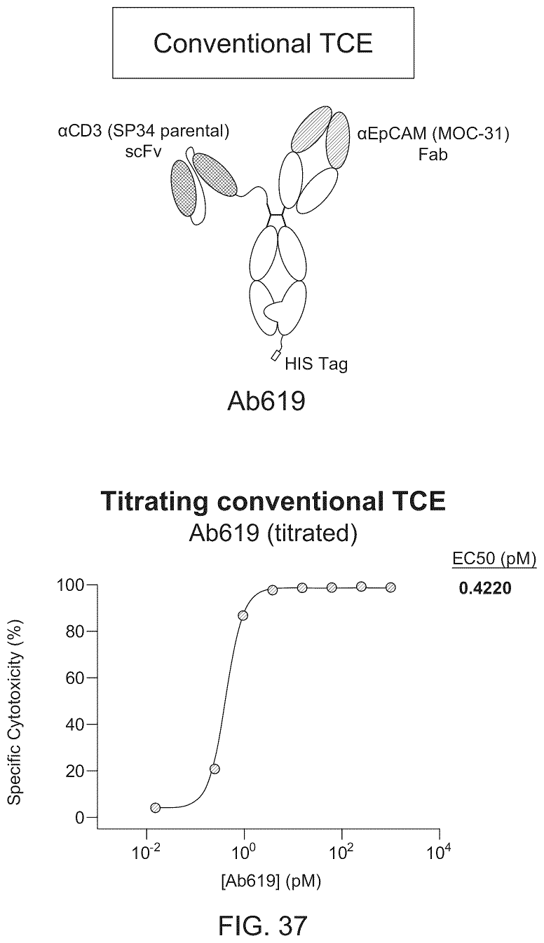

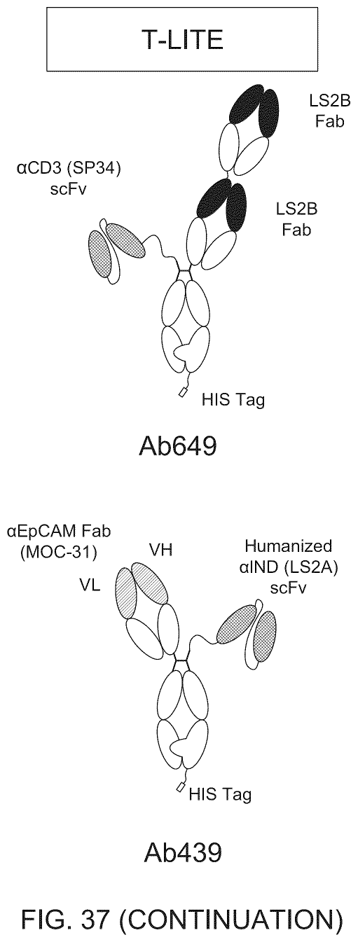

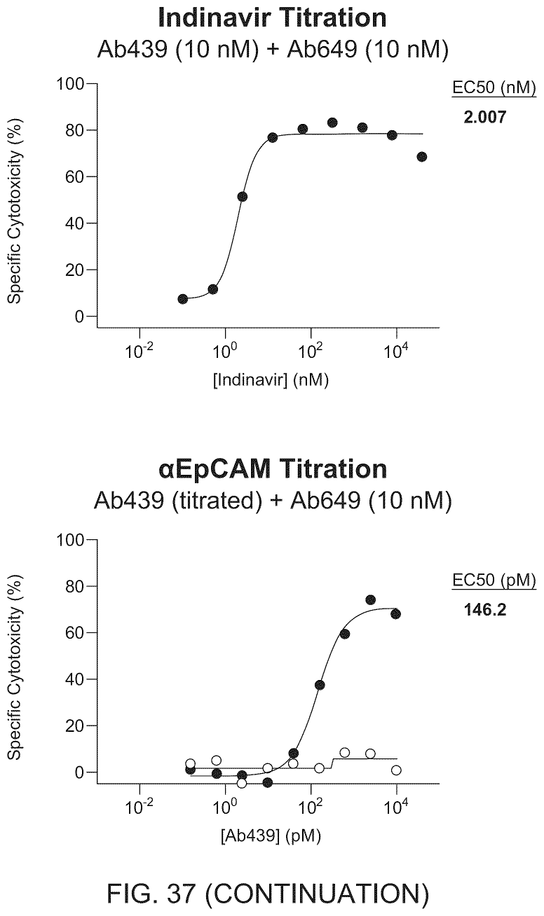

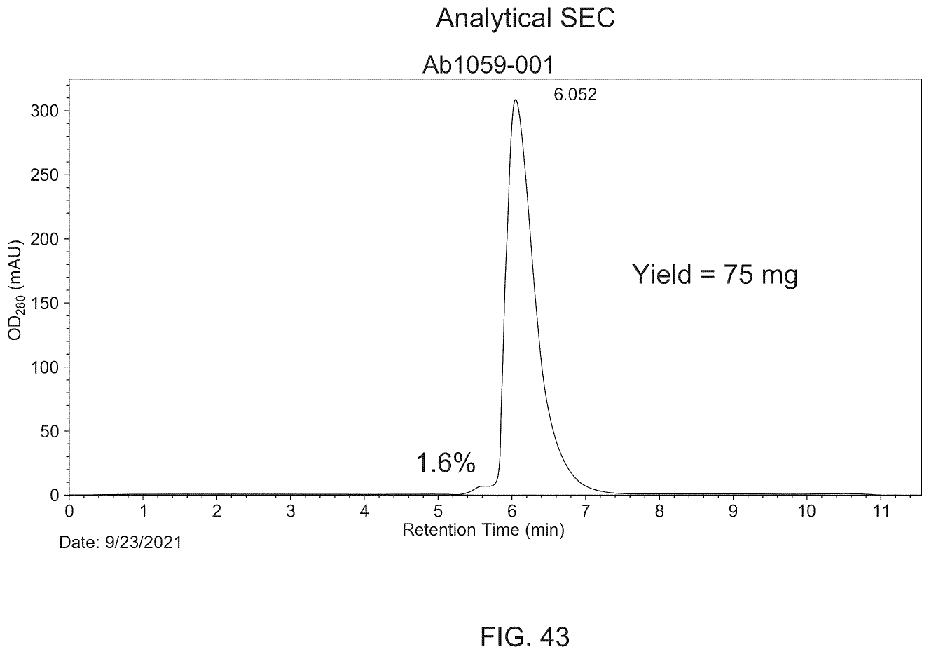

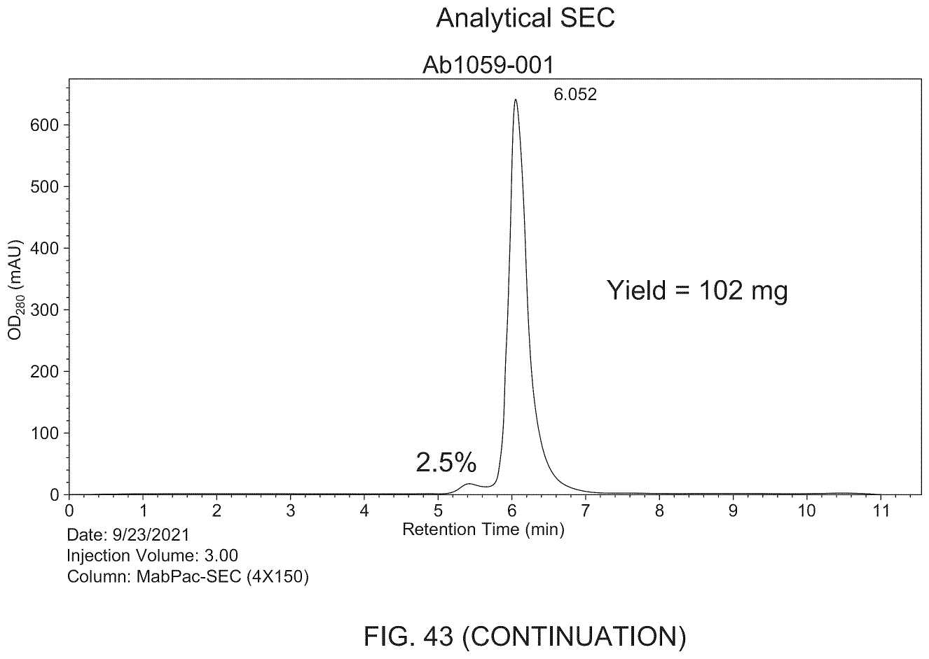

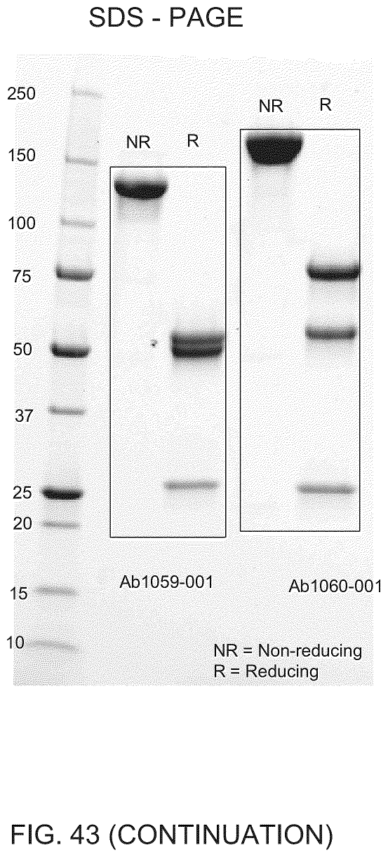

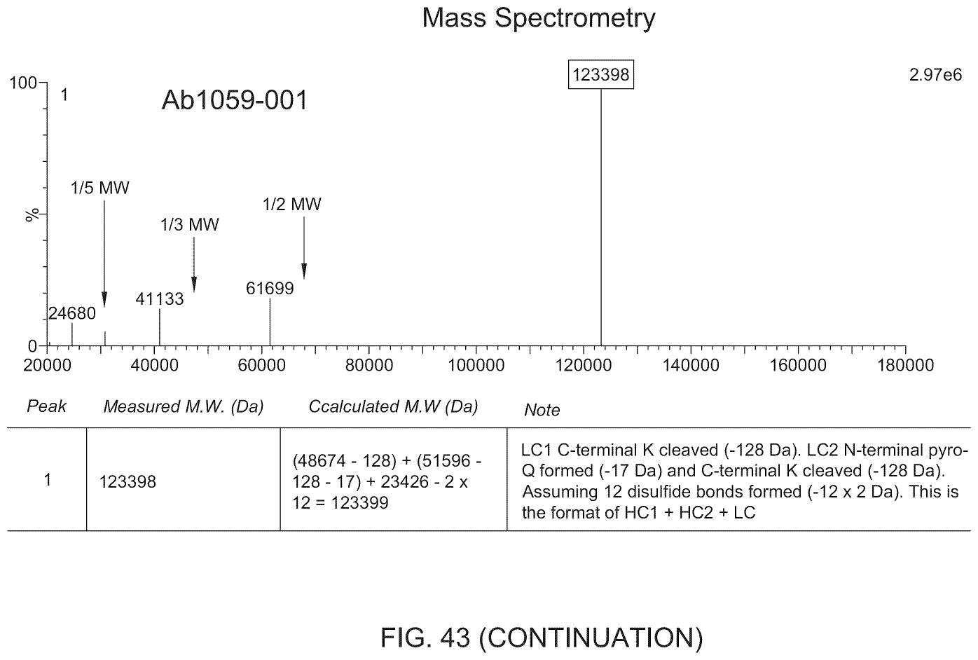

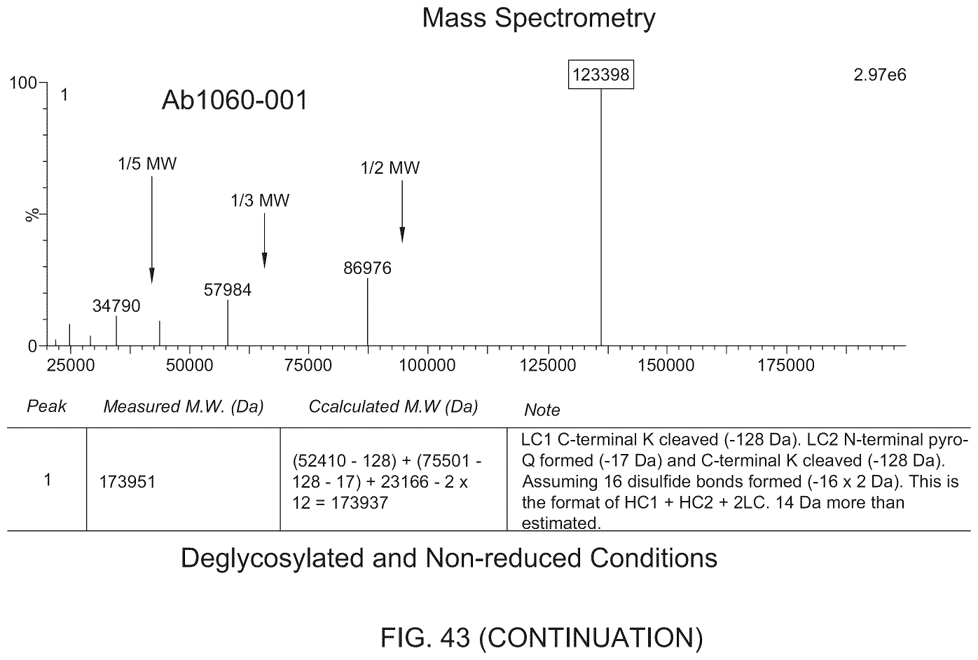

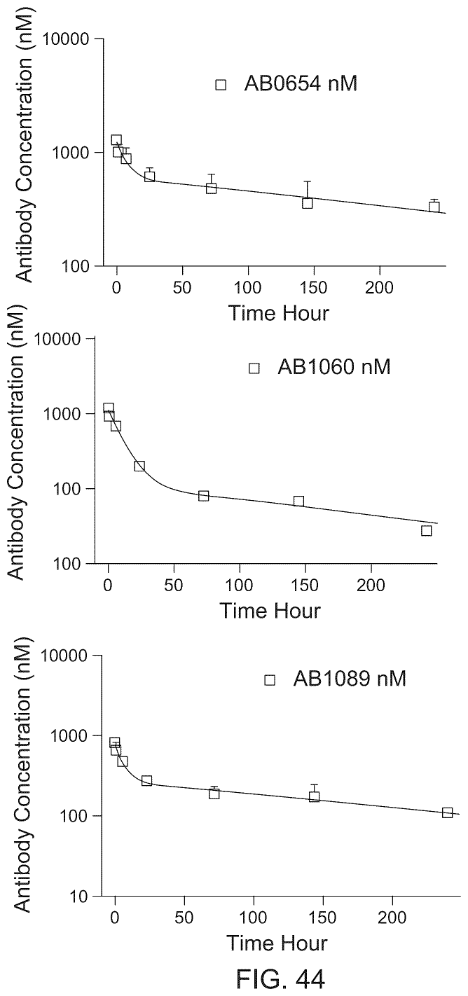

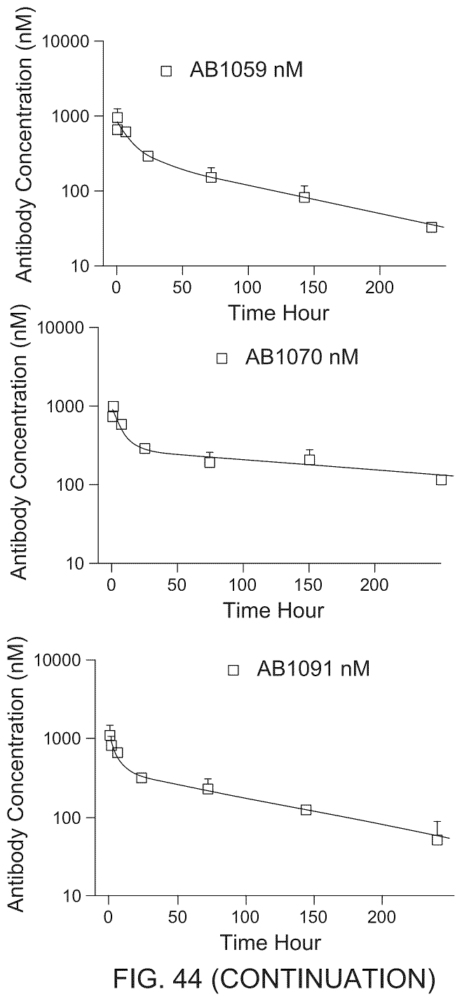

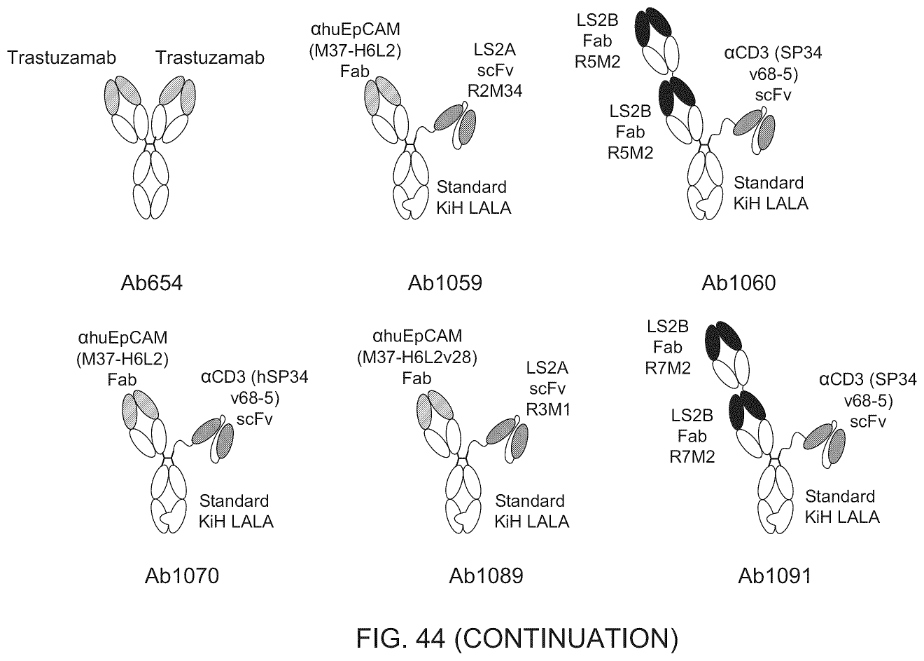

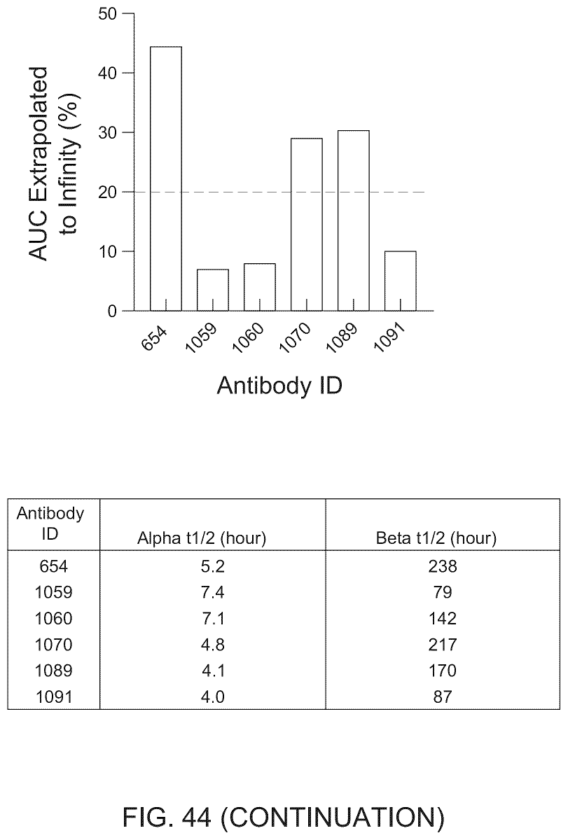

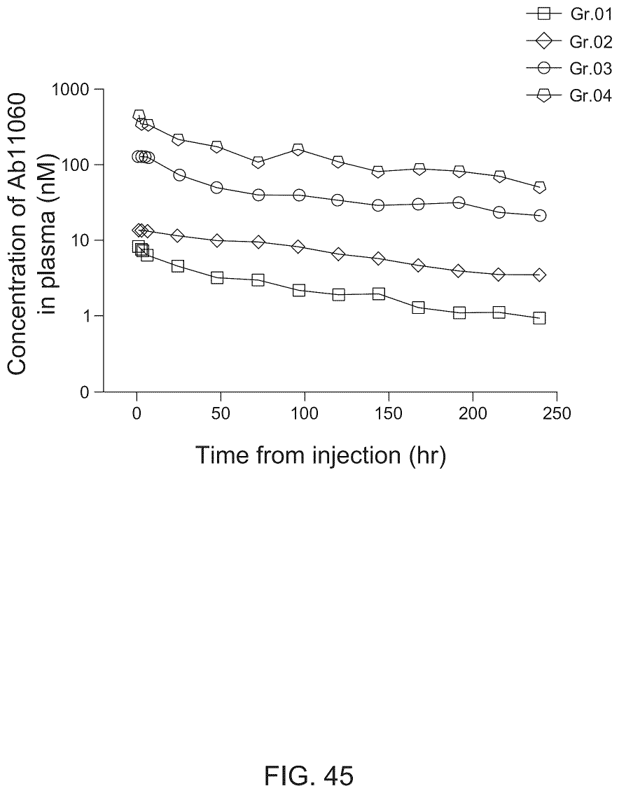

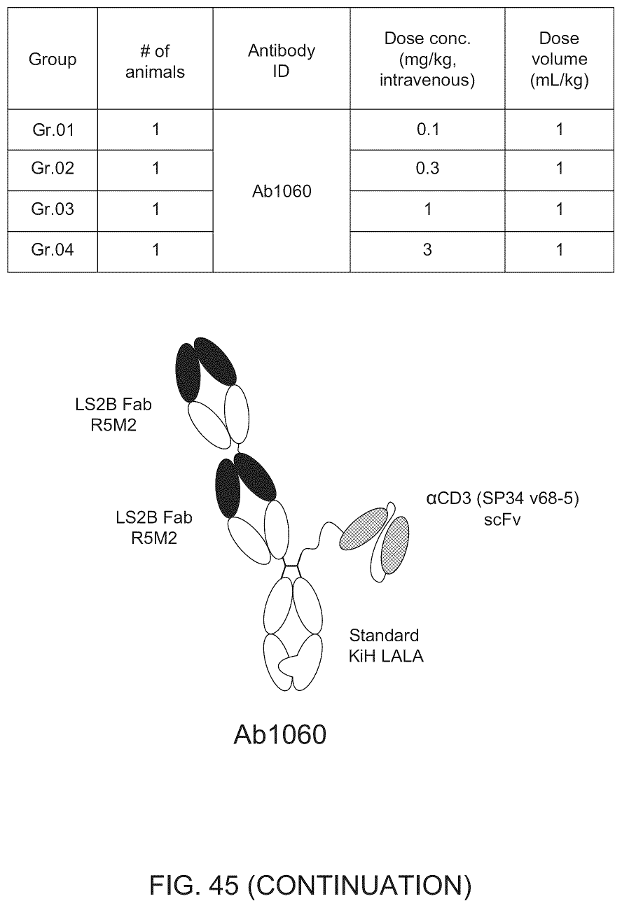

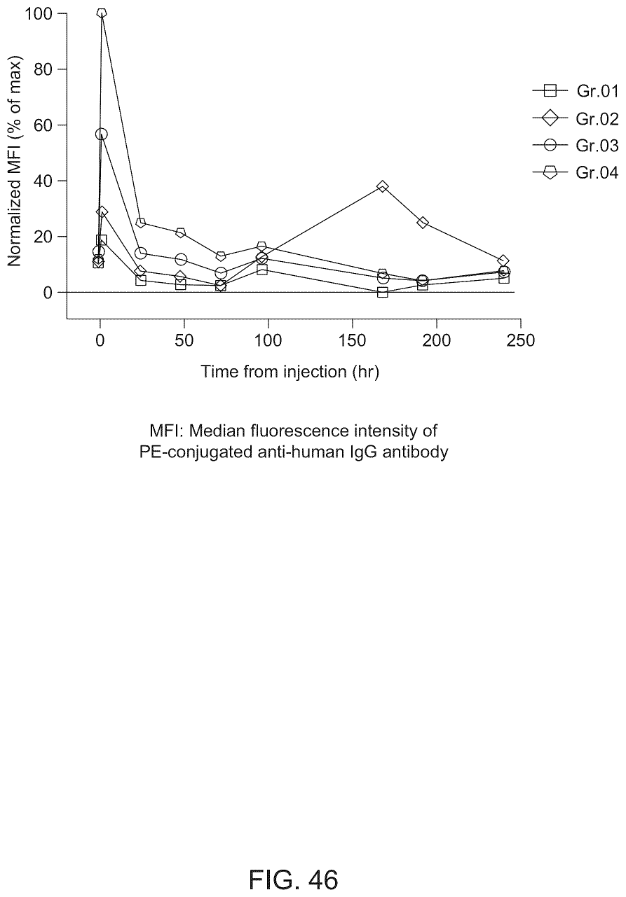

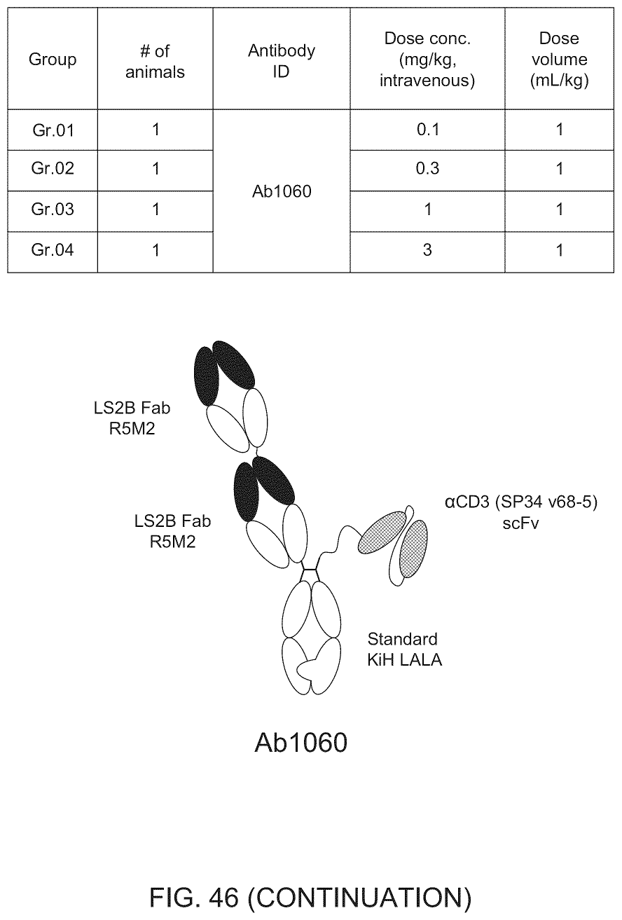

A panel of antibodies, including the 19 molecules depicted in (FIG. 36), was expressed and used to assess the effect of antibody format and geometry on the potency and switchable activity of EpCAM T-LITEs. Permutations explored in this panel of antibody formats included swapping whether the CC or CT antibody contained the LS2A or LS2B components of the LITE Switch. The panel also screened a range of valencies including 1+1 (CD3×EpCAM), 2+1 (CD3×EpCAM+EpCAM), and 2+2 (CD3+CD3×EpCAM+EpCAM) formats. The panel of antibodies was assessed in a series of TDCC experiments using MCF-7 target cells in a co-culture assay (FIG. 37). From this series of experiments, a pair of antibodies (Ab0439 and Ab0649) emerged with preferred combination of properties: Potent TDCC (146 pM EC50) of target cells in the presence of indinavir, no detectable TDCC in the absence of indinavir, and switchable TDCC requiring nanomolar concentrations of indinavir (2 nM EC50). This pair was a 2+1 format in which the first half of the T-LITE (the CC T-LITE) contained an anti-EpCAM Fab and an anti-indinavir scFv (LS2A), and the second half of the T-LITE (the CT T-LITE) contained an anti-CD3 scFv and two copies of the LS2B clone in a tandem Fab configuration. Example antibodies in this preferred format, Ab1059 and 1060, were well expressed (75 mg/L and 102 mg/L respectively), showed monodisperse peaks by UHPLC-SEC, and showed expected MW by SDS-PAGE and mass spectrometry (FIG. 43). Antibodies with this preferred format were explored in mice to determine their pharmacokinetic properties. Ab1059, Ab0160, Ab1070, Ab0189, and Ab1091 exhibited long half-lives (within 3-fold of the Herceptin control Ab0654) consistent with their format as IgG1 Fc domain containing antibodies (FIG. 44). The pharmacokinetic properties of Ab1060 were further assessed in cynomolgus macaques and exhibited a plasma half-life of 4-6 days (FIG. 45). Binding of Ab1060 to T-cells in the peripheral blood of cynomolgous macaques at various time points after dosing was confirmed by flow cytometry (FIG. 46).

### The Format of EpCAM T-LITEs Affects Indinavir-Dependent Killing

Further iterations of the 2+1 format were explored using later versions of the anti-CD3, anti-EpCAM, LS2A and LS2B clones, and these were tested in a TDCC co-culture assay with HCT-116 target cells. Two exemplary pairs (Ab1093+Ab1091 and Ab1094+Ab1060) were developed which exhibited potent TDCC (<100 pM EC50) in the presence of indinavir, no TDCC in the absence of indinavir, and switchable TDCC when indinavir was titrated (0.3-0.5 nM EC50) (FIG. 38). Notably, a significant amount of indinavir-independent killing was observed when the antibody clones in the CC T-LITE were swapped to the other heterodimeric Fc heavy chain (e.g. Ab1059 instead of Ab1093; Ab1089 instead of Ab1094), despite being paired with the same CT T-LITE antibodies (FIG. 39). Taken together the data supports that the αTTABD domain of the CT and the αCD3 domain of the CC need to either both be on the Fc polypeptide chain containing the “knob” point mutation, or both be on the chain containing the “hole” mutation of their respective heterodimers. This mismatch helps to prevent spontaneous formation of active conventional TCE when CC and CT are mixed in the absence of small molecule, in this case indinavir. This format enables the most switchable activity with the largest off/on dynamic range.

### Cytokine Production by EpCAM T-LITEs in Co-Culture Assays

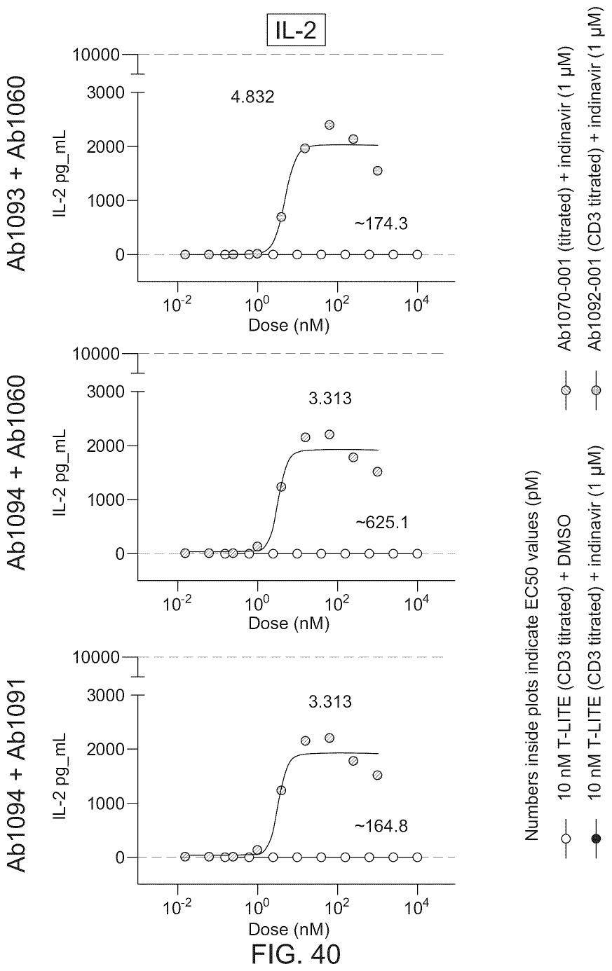

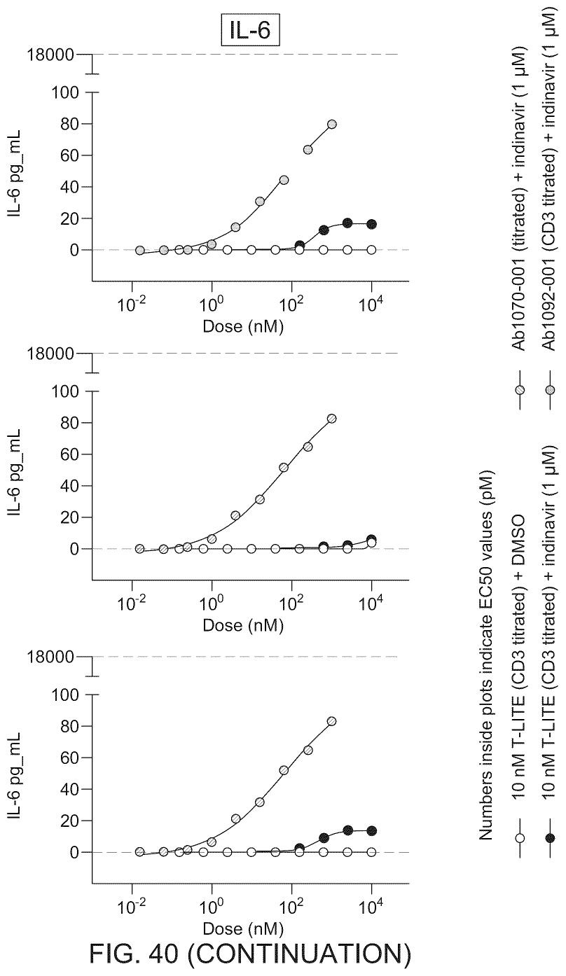

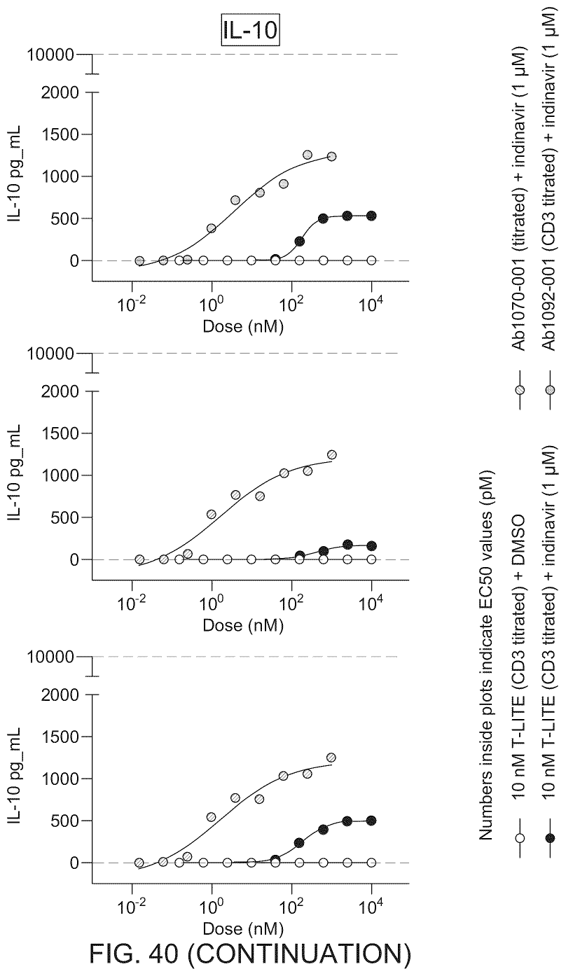

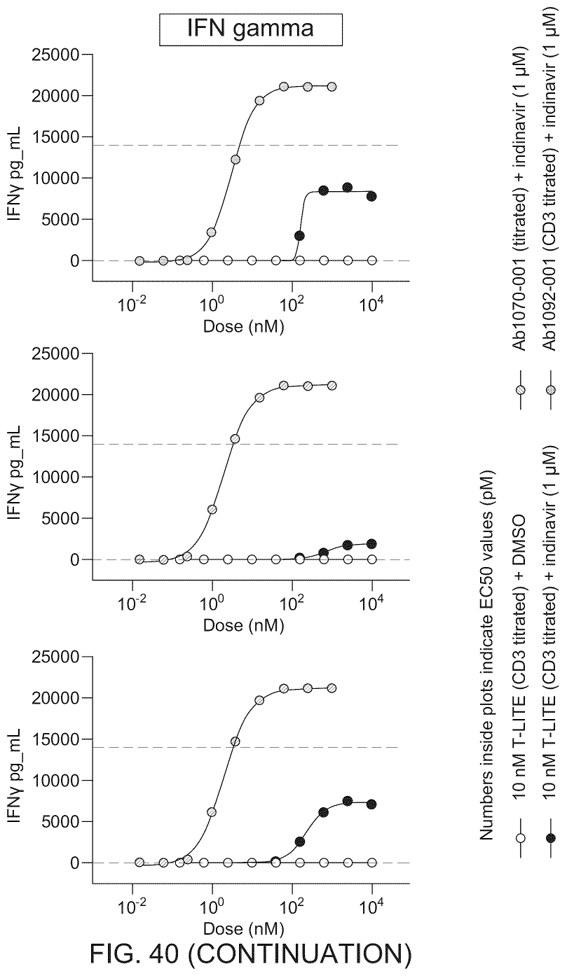

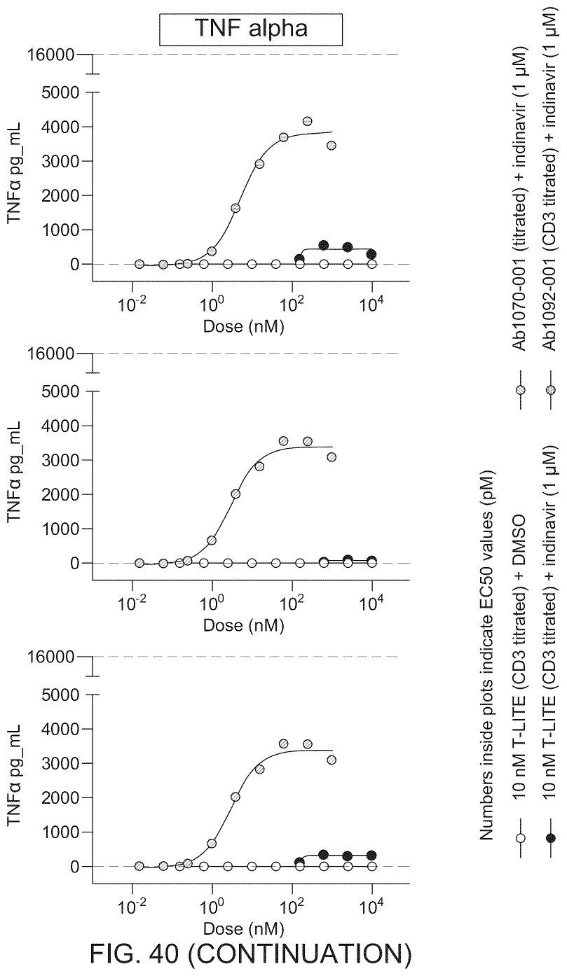

A bead-based multiplexed cytokine quantification system (LegendPlex) was used to measure the amount of cytokines released into the growth media by T-cells during TDCC co-culture assays. The supernatants in this experiment were taken from the same experiment as (FIG. 38). In a conventional TCE format, the M37 and hSP34 clones induced high levels of IL-2, IL-10, interferon (IFN) gamma, and TNF alpha (FIG. 40 and FIG. 41). The T-LITE pairs containing these same clones produced much lower levels of cytokines, even at doses that were sufficient to drive TDCC. Interestingly, the levels of cytokines varied depending on the chosen pair of CT and CC domains. Taken together, these data demonstrate that T-LITEs drive less cytokine production than conventional TCEs and the degree of cytokine production is decoupled from the degree of killing.

### Antigen Density Sensing by EpCAM T-LITEs in Co-Culture Assays

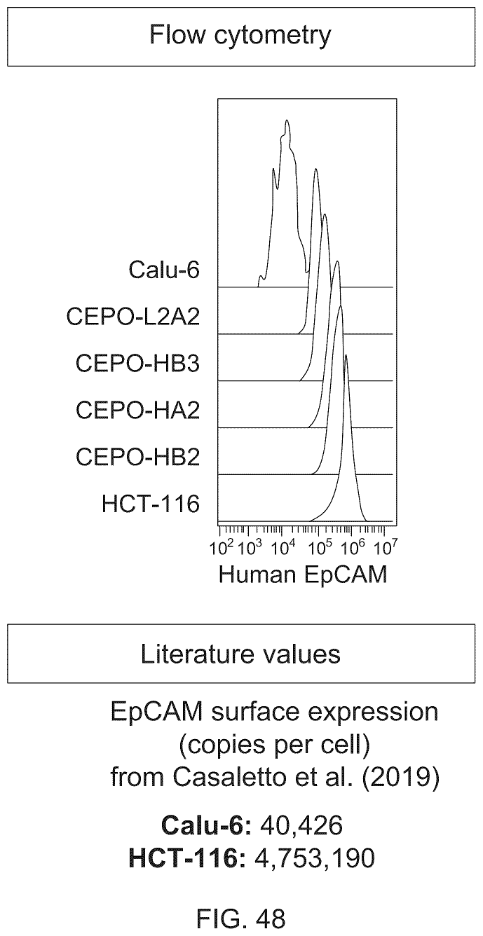

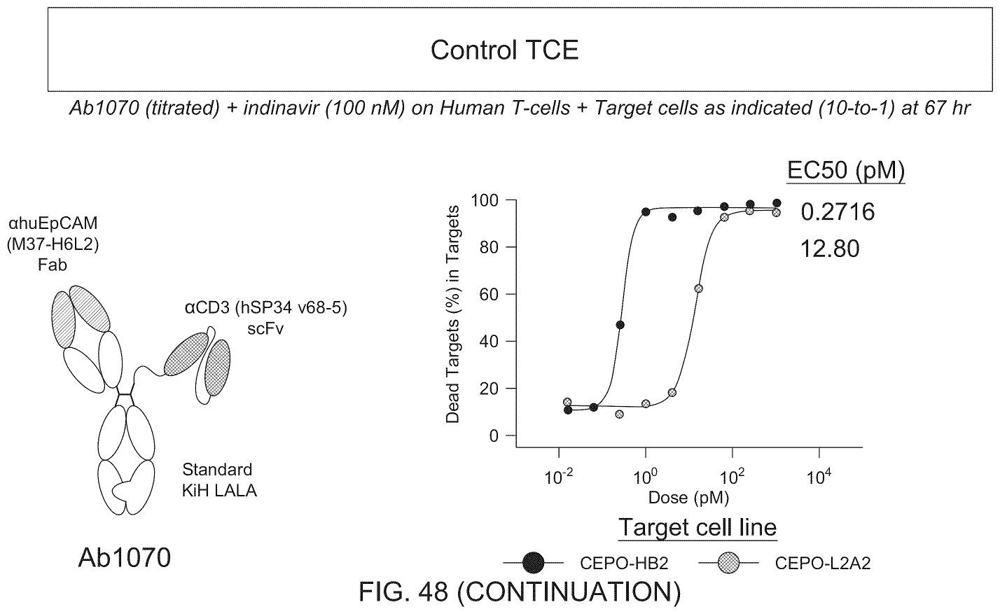

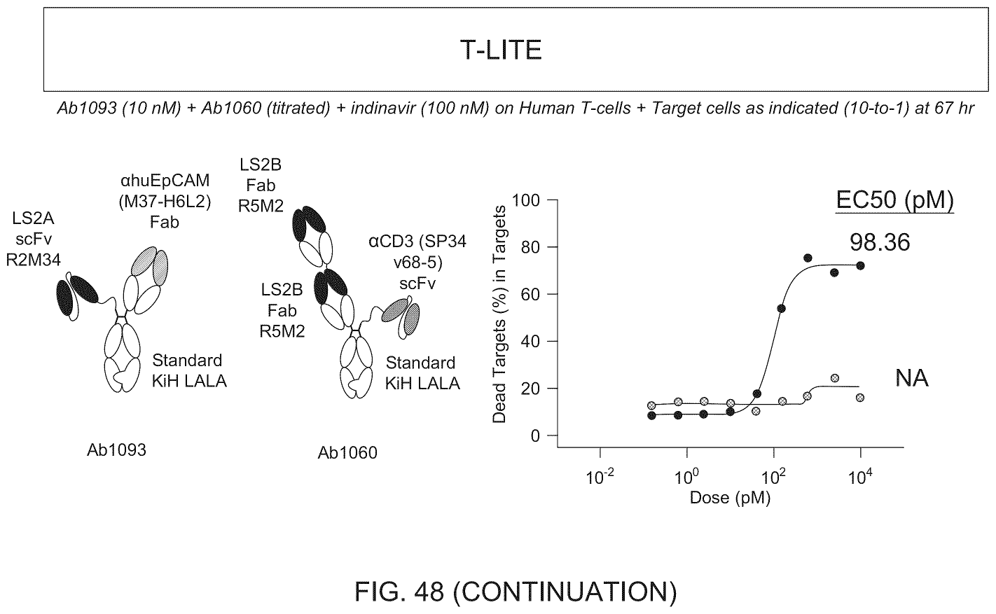

The engineered ability of a therapeutic modality to preferentially target high-expressing target cells while sparing low-expressing cells has been termed “antigen density sensing” in the literature and is a desirable property in some scenarios, especially cancer targets that are expressed at low levels on normal tissues (Choe et al., Ann Rev Cancer Biol 2020; doi: 10.1146/annurev-cancerbio-030419-033657). An exemplary T-LITE pair (Ab1060+Ab1093) and a control TCE with the same anti-EpCAM and anti-CD3 clones (Ab1070) were tested in a TDCC co-culture assay to explore how in vitro potency relates to antigen density on the surface of target cells (FIG. 48). The Ab1070 potently killed both target cell lines, with a window of 47-fold between the EC50 for the high- and low-expressing lines. The T-LITE exhibited dose-dependent killing of the CEPO-HB2 line (EC50: 98.36 pM). Notably, the T-LITE did not kill the CEPO-L2A2 line, even at the highest dose tested (10 nM). Overall, this data demonstrates that activated T-LITEs exhibit antigen density sensing capabilities, and preferentially kill high-expressing target cells.

### Tolerability of a CD3 T-LITE in Transgenic B-hCD3e Mice

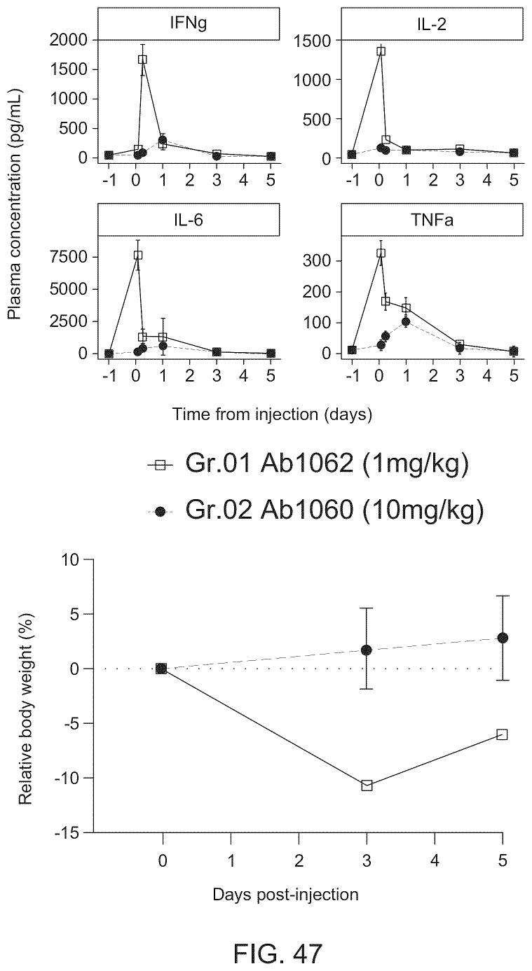

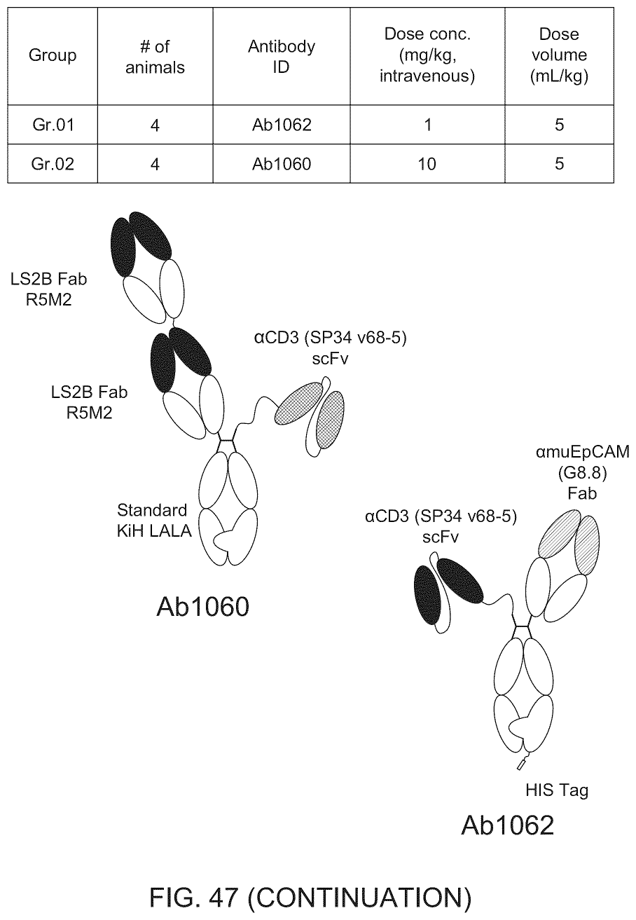

Transgenic mice expressing the extracellular domain of the human CD3E gene were used to assess the tolerability of a CD3 T-LITE incorporating the SP34v68-5 clone and LS2B Fab R5M2 (Ab1060) (FIG. 47). A separate group of mice was injected with a surrogate conventional TCE incorporating the anti-mouse the SP34v68-5 clone and the anti-mouse EpCAM clone G8.8 (Ab1062) as a positive control for T-cell activation. This positive control was expected to be poorly tolerated because it has been previously shown that repeated administration of a surrogate EpCAM TCE incorporating clone G8.8 was toxic in mice (Amann et al., J Immunother. 2009; doi: 10.1097/CJI.0b013e3181a1c097). After a single dose of Ab1062 (1 mg/kg), mice exhibited an average of 10% body weight loss by day 3 post-injection, whereas a single dose of Ab1060 (10 mg/kg) was well tolerated with no evidence of body weight loss within 5 days post-injection. The body weight loss associated with Ab1062 was correlated with a spike in cytokine release into the peripheral blood, as measured by a multiplexed cytokine assay. IFNg, IL-2, IL-6 and TNFa were all elevated by 6 hours post-injection in mice treated with Ab1062, whereas cytokine levels following Ab1060 injection either showed smaller changes or were unchanged, depending on the cytokine. Overall, this data demonstrates that the CD3 T-LITE Ab1060 did not cause widespread T cell activation in vivo in transgenic B-hCD3e mice and it was well tolerated as reflected by the lack of any overt clinical symptoms and the maintenance of body weight.

Blocking Indinavir Binding by LS2A with a Quencher Prevents Killing of Target Cells

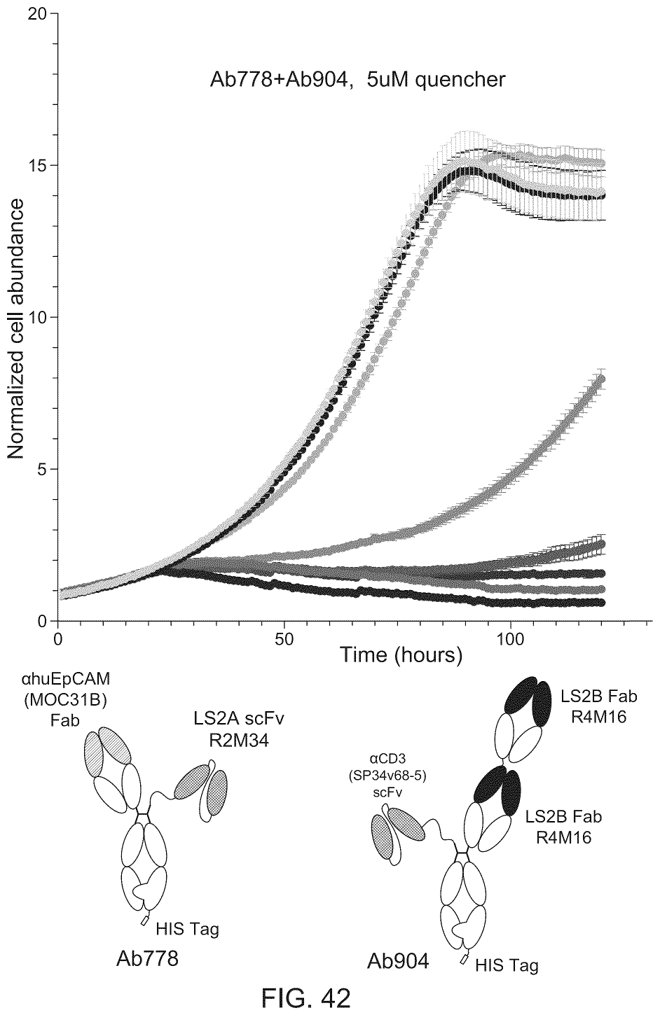

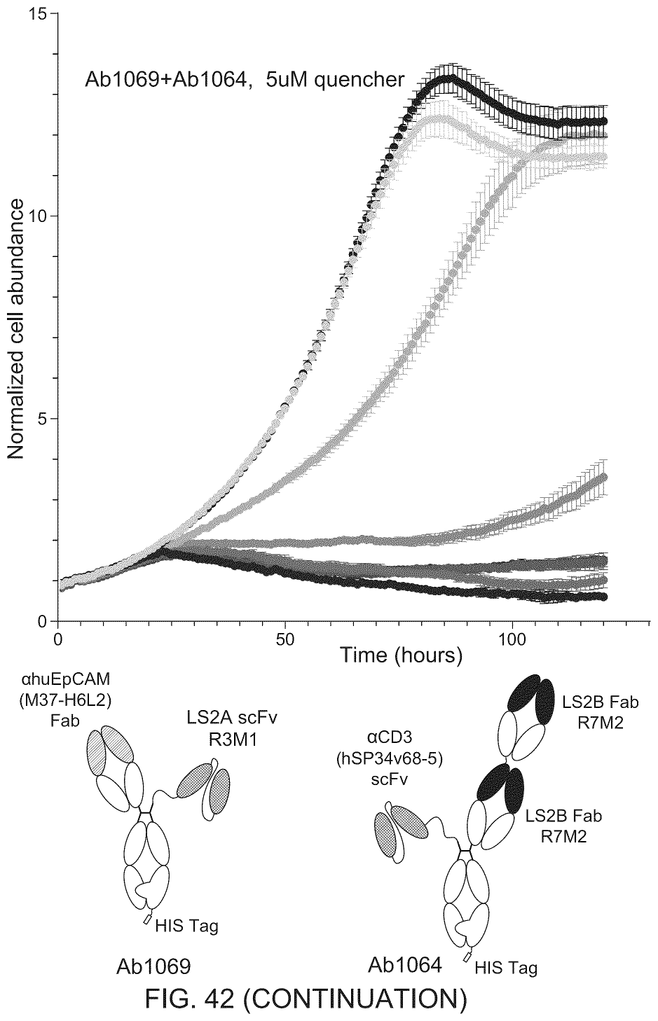

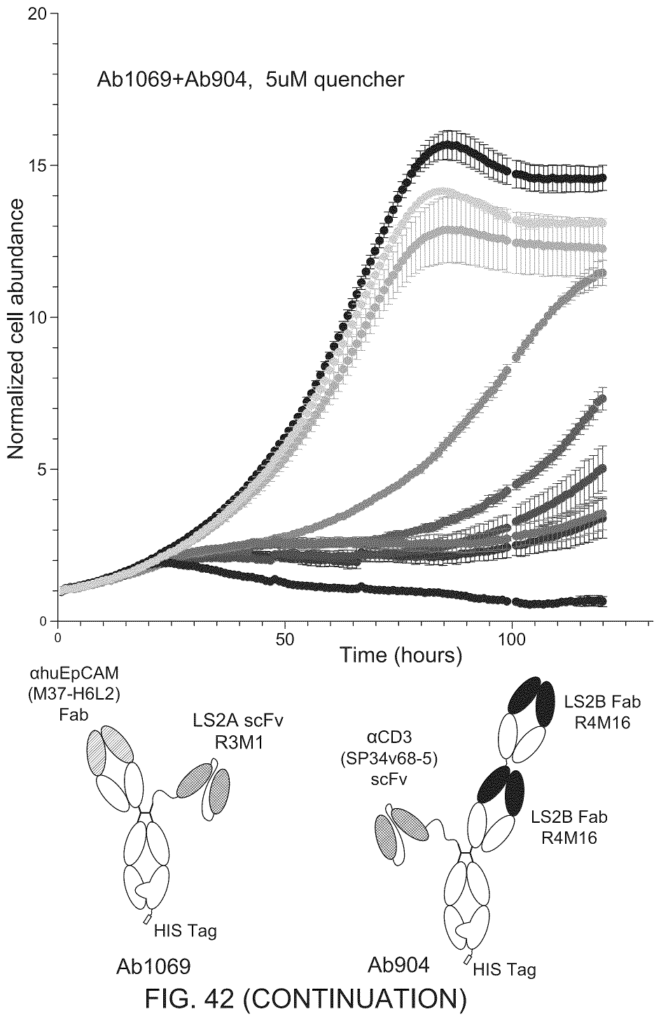

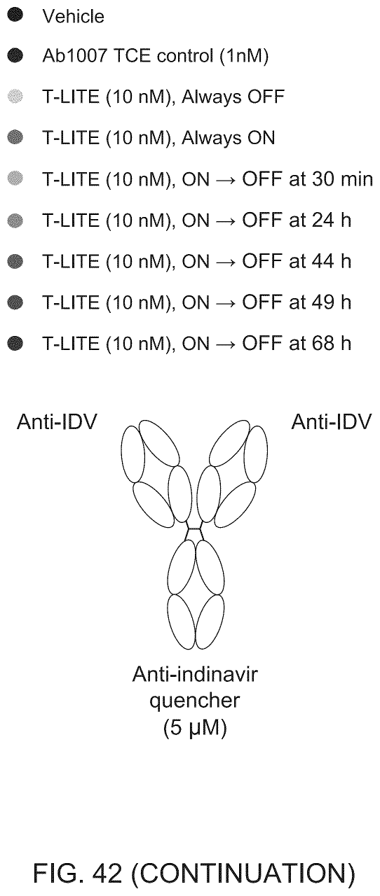

The kinetics of TDCC in a co-culture assay were measured using time-lapse microscopy (FIG. 42). The HCT-116-NR target cells exhibited unchecked growth in wells with the vehicle or T-LITEs in the absence of indinavir (“Always OFF”), consistent with the observation in other assays that T-LITEs require indinavir to induce T-cell activation. When indinavir was added at the earliest time point of T=0 min (“Always ON”), the plate was depleted of target cells, in a similar fashion as the conventional TCE (Ab1007), therefore demonstrating that the addition of indinavir can activate the T-LITE. To test how T-LITE-activated T-cells respond to the removal of indinavir, an “anti-indinavir quencher” was used. The anti-indinavir clone in the quencher is distinct from LS2A but binds to indinavir with a higher affinity than LS2A does, and it competes for the same binding epitope. Therefore, when the quencher is added in molar excess of LS2A (in this experiment, 5 μM quencher vs. 10 nM LS2A), it sequesters free indinavir and prevents its incorporation into new T-LITE complexes (“ON→OFF”). When the T-LITE was turned OFF at 30 min, nearly all TDCC was blocked, with the curve resembling the “Always OFF” condition. As the T-LITE was turned off at later time points, the tumor growth curves more closely resembled the “Always ON” condition. Taken together, these results demonstrate that the TDCC mediated by T-LITEs is switchable (it requires indinavir) and reversible (it can be disrupted by removing available indinavir from the system).

### Methods

### Vector Generation for Antibody Expression:

Plasmids for antibody expression were constructed by standard molecular biology methods. All DNA fragments were synthesized by IDT Technologies or Twist Biosciences and subcloned into either the pFUSE vector (InvivoGen) or pcDNA3.4 vector (Thermo Scientific) with Gibson assembly.

### Antibody Expression and Purification (FIG. 43):

All antibodies were expressed and purified from Expi293F (Thermo Fisher Scientific) or modified Expi293 BirA-KDEL (PMID: 29359686) cells according to an established protocol from the manufacturer (Thermo Fisher Scientific). Briefly, pFUSE (InvivoGen) vector encoding the protein of interest was transiently transfected into Expi293F cells at a fixed density of 3M/mL using the Expifectamine transfection kit and manufacturer's protocol (Thermo Fisher Scientific). Expression volumes varied, but a fixed vector:transfection reagent ratio (1 mg:2.8 ml) was used. For multi-chain proteins, a stoichiometric equivalent of each respective vector was used. Culturing media for BirA cells was additionally supplemented with 100 μM of biotin prior to transfection for in vivo biotinylation. Enhancing supplements from the Expifectamine kit were added 20 hours post-transfection. Cells were incubated for a total of 4 d at 37° C. in an 8% CO₂ environment with orbital shaking before the supernatants were harvested by centrifugation. Fc-fusion proteins were purified by Protein A (MabSelect PrismA) affinity chromatography and His-tagged proteins were purified by Ni-NTA (Roche cOmplete™ His-Tag Resin) affinity chromatography. Eluted proteins from affinity purification were further separated by Size-exclusion chromatography with a Superdex 200 increase 10/300 GL column (Cytiva) in storage buffer (1×HBS+5% Glycerol) as an aqueous phase. Samples were injected manually and fractionated via an AKTA Pure System with Software UNICORN 7.3 (Cytiva). Purity and integrity were assessed by SDS-PAGE with 4-12% BIS-TRIS precast gels (Thermo Fisher Scientific). Samples were stored at either 4° C. or −80° C. with storage buffer in aliquots.

### Differential Scanning Fluorimetry (DSF) (FIG. 19):

DSF was performed on a LC480 Lightcycler Instrument II (Roche). Purified recombinant protein was diluted to 5 μM in DSF buffer (PBS, pH 7.4, Sypro Orange 5×) with small molecule (10 μM VTX) or vehicle (0.05% DMSO) and then heated with a temperature gradient (0.01° C./s) from 25 to 95° C. Data were continuously acquired at ˜465 nm (excitation) and ˜580 nm (emission). Data was analyzed to generate first derivative curves in which the curve maximum was reported as the melting temperature of the protein.

### Ultrahigh Pressure Liquid Chromatography—Size Exclusion Chromatography (UPLC-SEC) (FIG. 15, FIG. 16, FIG. 18, FIG. 43):

UPLC-SEC was performed on a Thermo Scientific Vanquish Flex UHPLC system. Protein was diluted to 1-5 μM in HBS buffer and then 1.5-10 μL of sample was injected onto a MabPAC 4.6×150 mm in 1×PBS-HCl at pH 7.0. Data were continuously acquired at ˜280 nm and processed on the Chromeleon 7 software (Thermo Scientific). For the low pH hold assay, prior to injection on the UHPLC, samples were held at pH 3.5 for 1 hour followed by neutralization. For the complex assembly assay, the MabPAC 4.6×150 mm column was pre-equilibrated with 1×PBS-HCl at pH 7.0 and 10 μM Indinavir.

### Mass Spectrometry (FIG. 43):

Sample was denatured with or without reducing by guanidine and DTT and then deglycosylated by PNGaseF (MEDNA Bio E1041). The sample was analyzed by Waters ACQUITY UPLC coupled to XevoG2-XS QTOF mass spectrometer using an ACQUITY UPLC Protein BEH SEC column.

### Binding Kinetics Analysis (FIG. 17 and FIG. 23):

Bio-layer interferometry (BLI) data were measured using an Octet RED384 (ForteBio) instrument. Antigens of interest were immobilized on an AHC biosensor and loaded until a 0.4-0.6-nm signal was achieved. Purified antibodies were used as the analyte premixed with 10 μM Indinavir or 0.05% DMSO vehicle. PBSTB (PBS, pH 7.4, 0.05% Tween-20, 0.2% BSA) was used for all buffers for BLI. Data were analyzed using ForteBio Octet analysis software and kinetic parameters were determined using a 1:1 monovalent binding model.

Supertant titer quantitation on SP34 clones (FIG. 28) was performed accordingly. Briefly, supernatents from 24-well plates were harvested, centrifuged at 4100 g for 20 minutes to pellet expression cells, and clarified supernatents were transferred to an Octet 96-well plate. AHQ biosensors were used with standard manufactures protocol for sample quantitation to measure antibody concentrations. Sample concentrations were extrapolated from a standard curve generated with an IgG reference molecule diluted in Expi293F media. For detection of CD3E binding in supernatents, biotinylated CDE antigen CD3E ECD (AcroBio) was immobilized on a streptavidin biosensor and loaded until ˜1 nm signal was achieved. Biosensors were then dipped in clarified cell supernatant to detect binding.

LITE Switch indinivir (IDV) washout/reversibility kinetics (FIG. 21) were measured by biolayer interferometry (BLI) on an Octet® Qke (Sartorius) instrument. Biotinylated scFv-Fc Ag67, in the presence of 1 μM IDV or 0.05% DMSO vehicle, was immobilized on streptavidin biosensors and loaded to a signal of 1.2-1.8 nm. After loading, biosensors were blocked with 10 μM biotin containing 1 μM IDV or vehicle. Association steps were performed for 10 minutes in the presence of 50 nM Ab1073 Fabs and 1 μM IDV or vehicle. At steady-state binding of Ab1073 to Ag67, LITE Switch complexes dissociated for 2 hours in buffer containing (1) 50 nM Ab1073 and 1 μM IDV, (2) 50 nM Ab1073 and vehicle (3) vehicle only. Data were analyzed using ForteBio Data Analysis HT software version 12.0.2.59. Dissociation constants were determined using a 1:1 monovalent binding model, local fitting, and with dissociation baseline to zero. Data were additionally analyzed using GraphPad Prism 9 software version 9.1.2 (225) with a one-phase exponential decay nonlinear regression model. Parameters included NS (binding at infinite times) set to zero and K (inverse rate constant)>0.

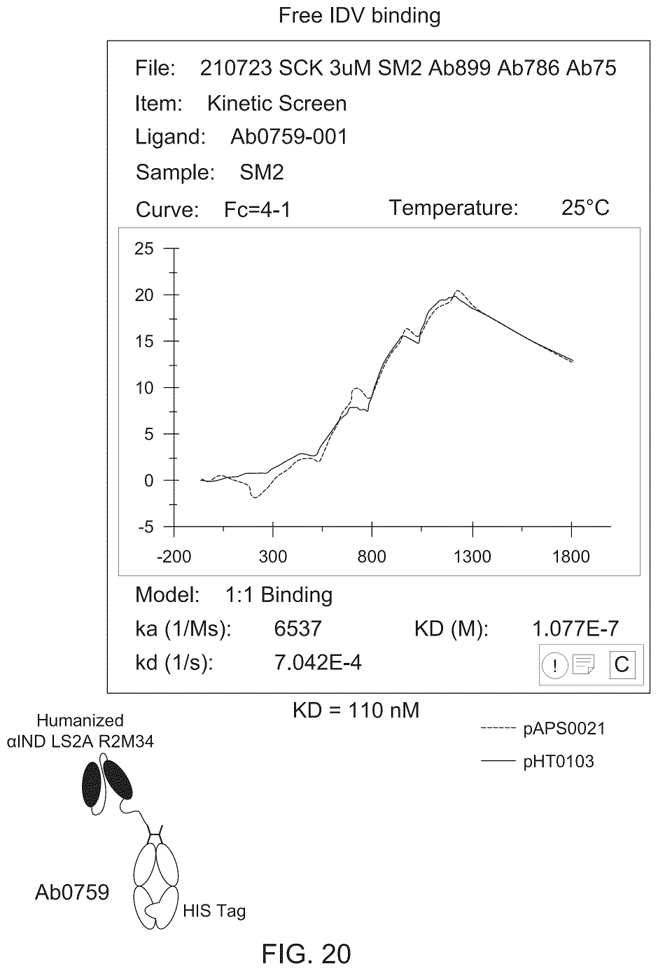

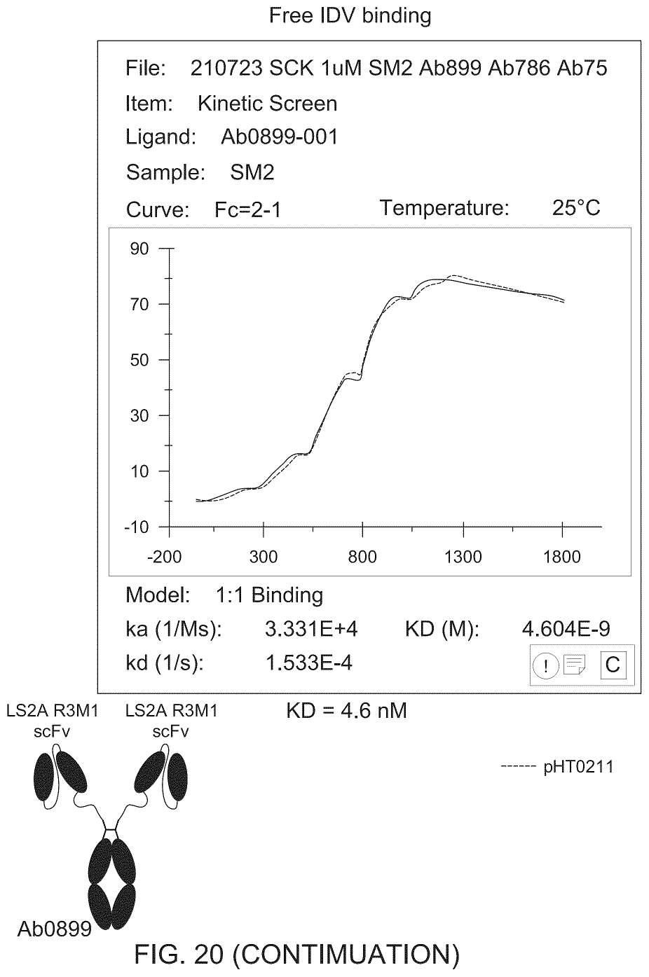

### Biacore (FIG. 20):

The affinities of anti-EpCAM antibodies to human or cyno EpCAM ECD were measured by surface plasmon resonance (SPR) with a Biacore T200. All assays were performed at 25° C. in 1×HBS-EP+ buffer made from a 10× buffer stock (Cytiva BR100669). Anti-EpCAM antibodies were captured on a Series S Sensor Chip CM5 (Cytiva 29149603) using the Human Antibody Capture Kit (Cytiva BR100839). For single-cycle kinetics experiments, five sequential injections of 3-fold serial diluted (1.23, 3.70, 11.1, 33.3, 100 nM) human (Ag0065) or cyno (Ag0066) EpCAM ECD antigen at 30 μL/min for 180 seconds each was performed, and dissociation was monitored for 600 seconds. The data was double referenced subtracted to the reference lane and five sequential injections of 0 nM antigen. For multi-cycle kinetics experiments, 0 nM antigen was injected for 300 seconds at 30 L/min followed by a dissociation step for 600 seconds. Five additional cycles were performed with increasing antigen concentrations of 1.23, 3.70, 11.1, 33.3, and 100 nM. The data was double referenced subtracted to the reference lane and the 0 nM sensogram. The sensor chip was regenerated with an injection of 3 M magnesium chloride for 45 seconds at 30 μL/min after each cycle. Single-cycle and multi-cycle data was fit to a 1:1 Langmuir model to determine the association and dissociation rate constants.

The affinities of anti-CD3e antibodies to monovalent human CD3e N-terminal peptide AA1-26 fused to a murine Fc domain (Ag0060) were measured by surface plasmon resonance (SPR) with a Biacore T200. All assays were performed at 25° C. in 1×HBS-EP+ buffer made from a 10× buffer stock (Cytiva BR100669). Anti-CD3e antibodies were captured on a Series S Sensor Chip CM5 (Cytiva 29149603) using the Human Antibody Capture Kit (Cytiva BR100839). For single-cycle kinetics experiments, five sequential injections of 3-fold serial diluted (3.70, 11.1, 33.3, 100, 300 nM) Ag0060 at 30 μL/min for 120 seconds each was performed, and dissociation was monitored for 420 seconds. For weakened affinity variants, the single-cycle experiment was modified to have five sequential injections of 3-fold serial diluted (24.7, 74.1, 222, 667, 2000 nM) Ag0060 at 30 L/min for 180 seconds each and dissociation was monitored for 600 seconds. The data was double referenced subtracted to the reference lane and five sequential injections of 0 nM antigen. The sensor chip was regenerated with an injection of 3 M magnesium chloride for 45 seconds at 30 μL/min after each cycle. The kinetic data was fit to a 1:1 Langmuir model to determine the association and dissociation rate constants.

The affinities of LS2A clones R2M34 (Ab0759) and R3M1 (Ab0899) to free indinavir were measured by surface plasmon resonance (SPR) with a Biacore T200. All assays were performed at 25° C. in 1×PBS-P+ buffer made from a 10× buffer stock (Cytiva 28995084). Ab0759 and Ab0899 were amine coupled to a Series S Sensor Chip CM5 (Cytiva 29149603). Trastuzumab, a non-binding control, was amine coupled to the reference lane. For Ab0759, a single-cycle kinetics experiment was performed with five sequential injections of 3-fold serial diluted (37, 111, 333, 1000, 3000 nM) indinavir at 30 μL/min for 240 seconds each and dissociation was monitored for 600 seconds. For Ab0899, a single-cycle kinetics experiment was performed with five sequential injections of 3-fold serial diluted (12.3, 37, 111, 333, 1000 nM) indinavir at 30 L/min for 240 seconds each and dissociation was monitored for 600 seconds. The data was double referenced subtracted to the reference lane and five sequential injections of 0 nM indinavir. The kinetic data was fit to a 1:1 Langmuir model to determine the association and dissociation rate constants.

### Measurement of Ab0640 Pharmacokinetics in Mice (FIG. 31):

Female C56BL/6 mice (age 10-12 weeks) were distributed into 2 groups (n=5 per group). Antibodies were diluted in sterile PBS and injected intravenously at 6.7 mg/kg. Blood (55 μL from the retroorbital sinus) was sampled serially at 4 time points (30 min, 1 day, 3 days, 7 days), processed to plasma, diluted 1:10 in 50% glycerol+PBS, then cryopreserved. Plasma concentrations were quantified using an anti-human IgG sandwich ELISA that was developed and qualified in-house. The capture antibody for the ELISA was goat anti-human IgG (Cat. No. 2049-01; Southern Biotech, Birmingham, AL) coated on 96-well Nunc-Immuno Maxisorp plates (Thermo Fisher Scientific) at 1 pg/mL for 1 hr at 37° C. The detection antibody was mouse anti-human IgG Fc-HRP (Cat. No. 9042-05; Southern Biotech, Birmingham, AL) at 1:32,000 dilution of the manufacturer stock. ELISA coating, blocking, wash, and TMB substrate buffers from BioLegend were used according to the manufacturer's instructions. Non-compartmental pharmacokinetic parameters were calculated using the PKNCA package for R (doi: 10.1007/s10928-015-9432-2).

### Measurement of Ab1059, Ab1060, Ab1070, Ab1089, and Ab1091 Pharmacokinetics in Mice (FIG. 44)

Female C56BL/6 mice (age 11-12 weeks) were distributed to 6 groups (n=5 per group). Antibodies were diluted in sterile PBS and injected intravenously at the molar equivalent of 10 mg/kg IgG (adjusted for the actual molecular weight of the antibody, and assuming a 150 kDa molecular weight for a typical IgG). Blood (25 μL from the tail vein) was sampled serially at 7 time points (15 min, 1 hr, 6 hr, 24 hr, 72 hr, 6 days, 10 days), processed to plasma, diluted 1:10 in 50% glycerol+PBS, then cryopreserved. Plasma concentrations were quantified using an anti-human IgG sandwich ELISA that was developed and qualified in-house as described above. Two-compartment pharmacokinetic parameters were calculated using the Phoenix WinNonLin software package (Certara, Princeton, NJ).

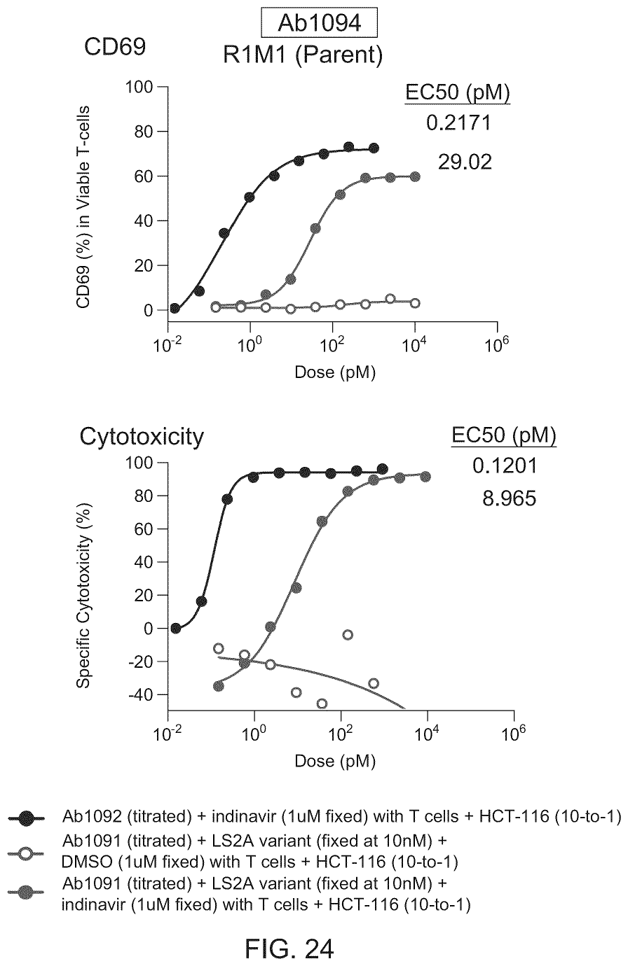

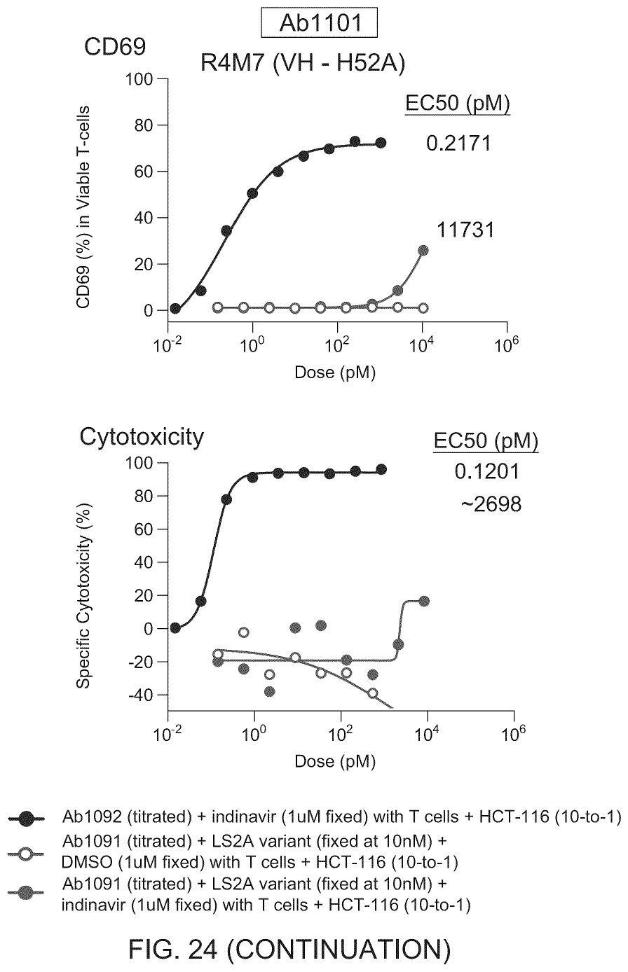

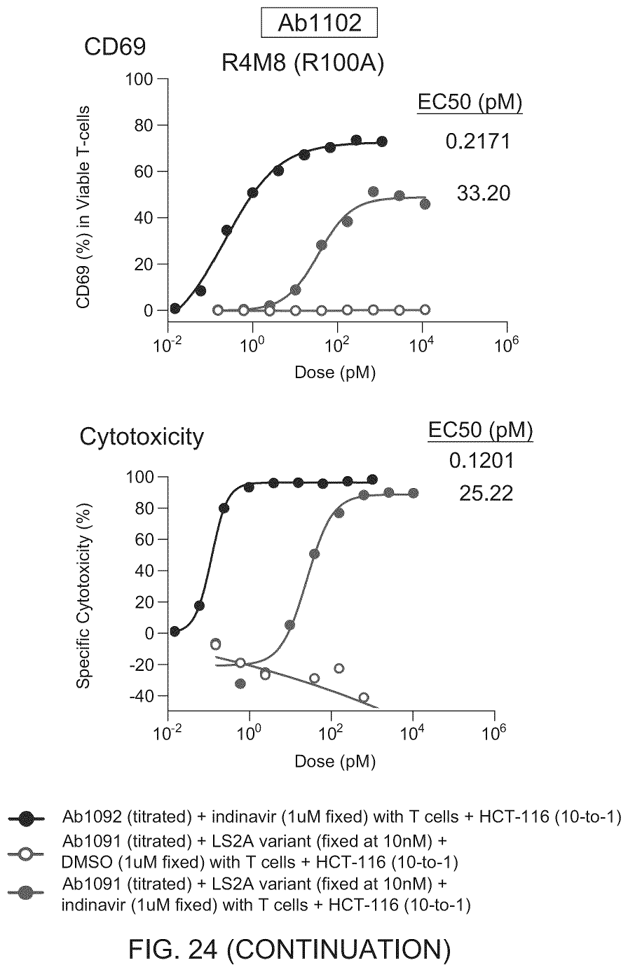

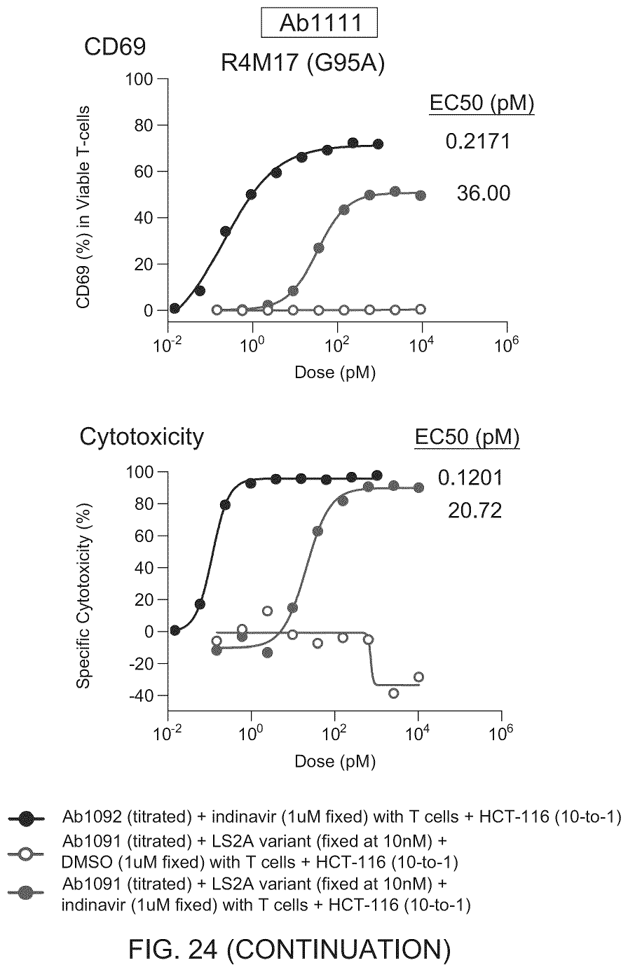

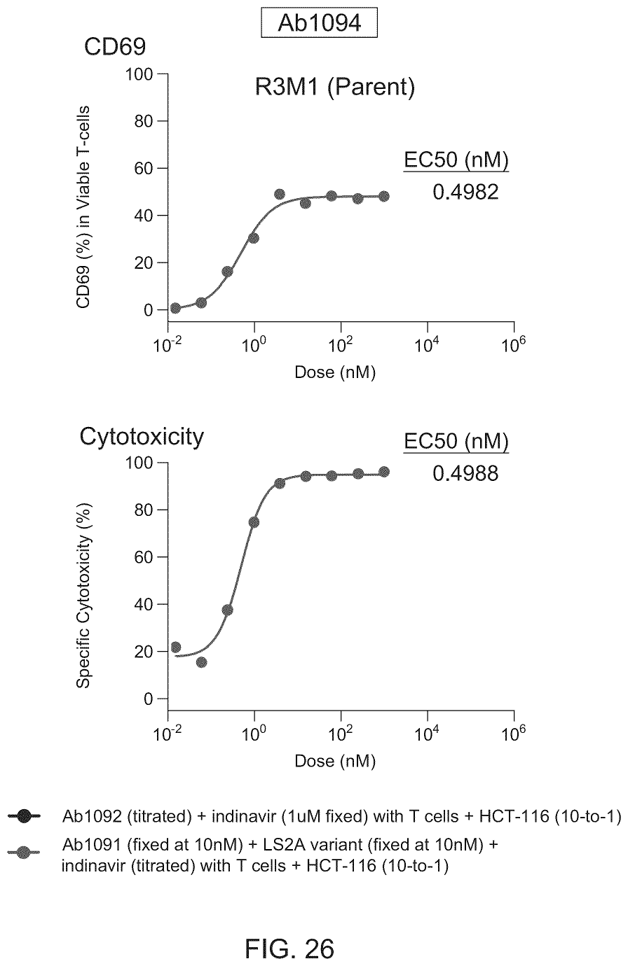

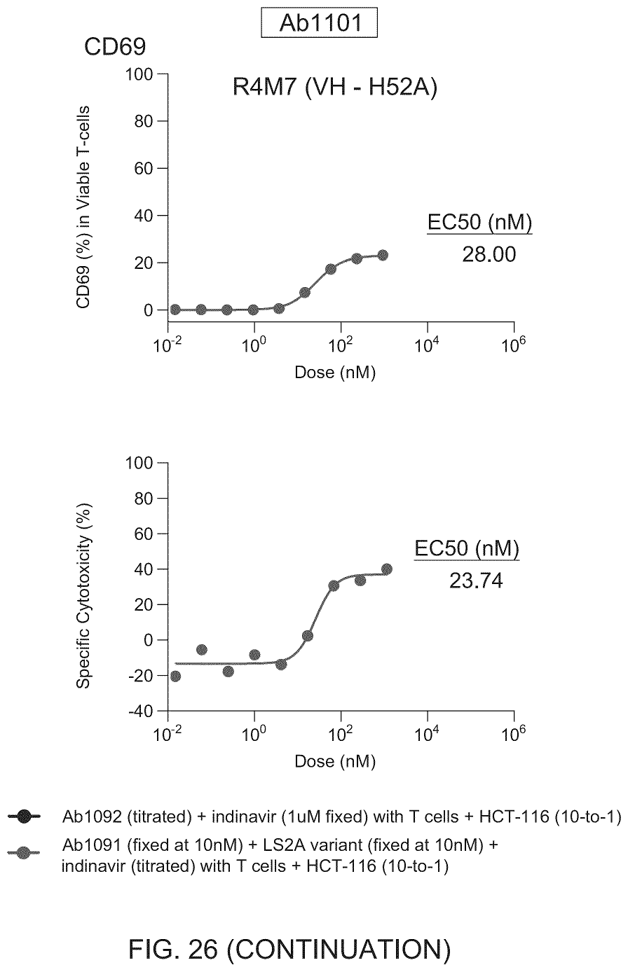

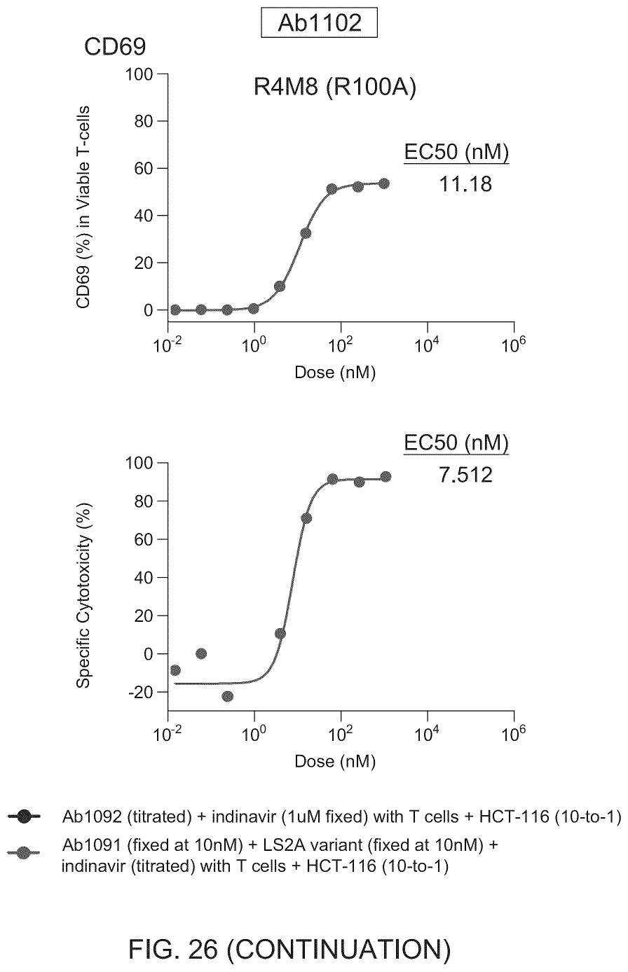

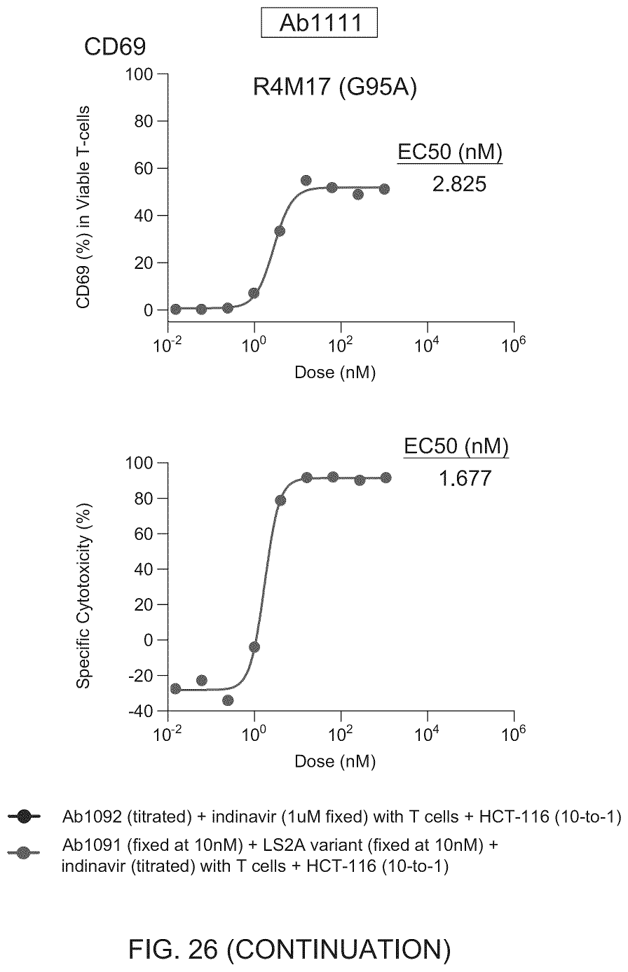

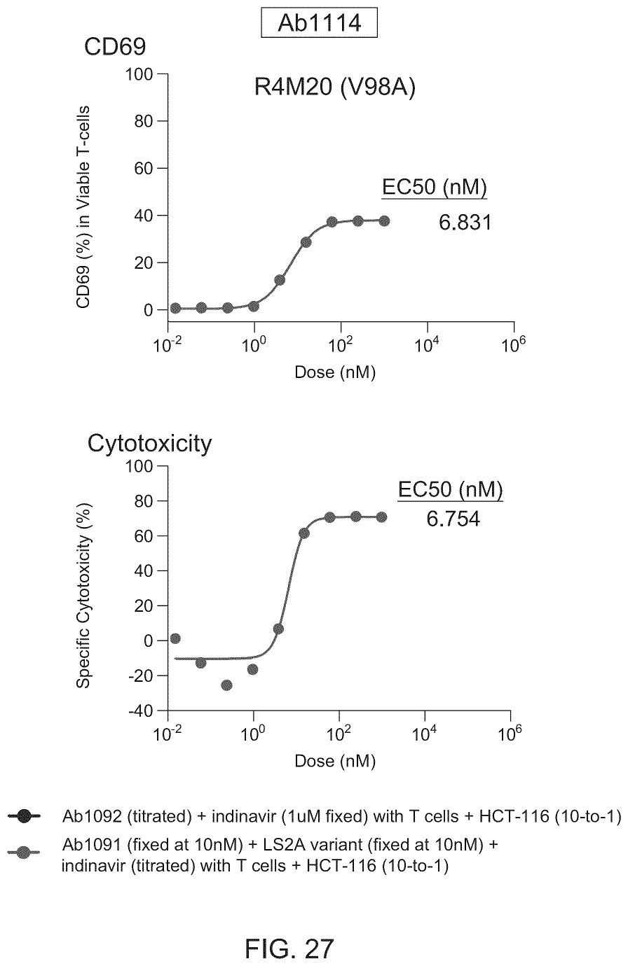

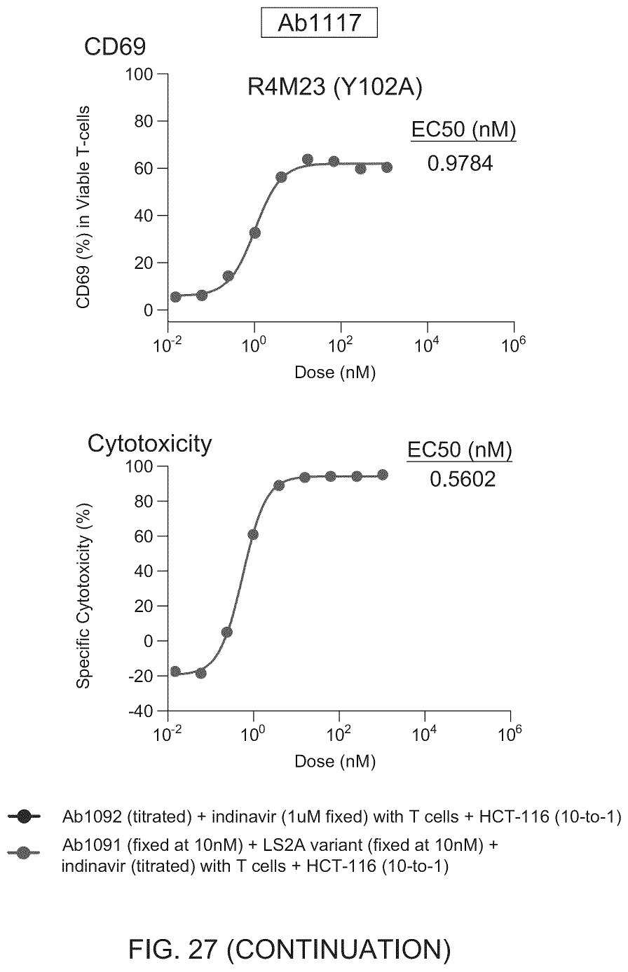

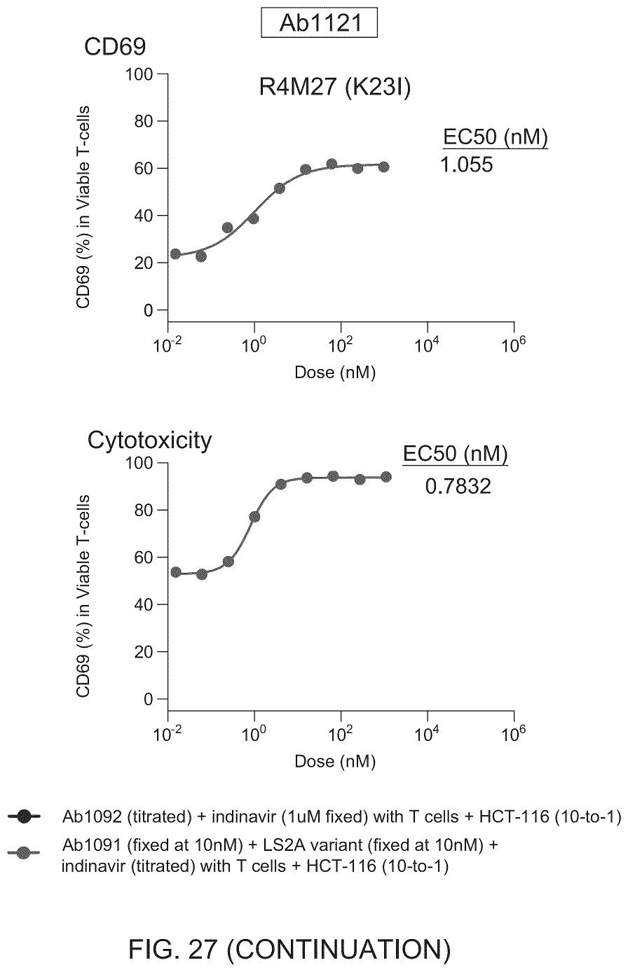

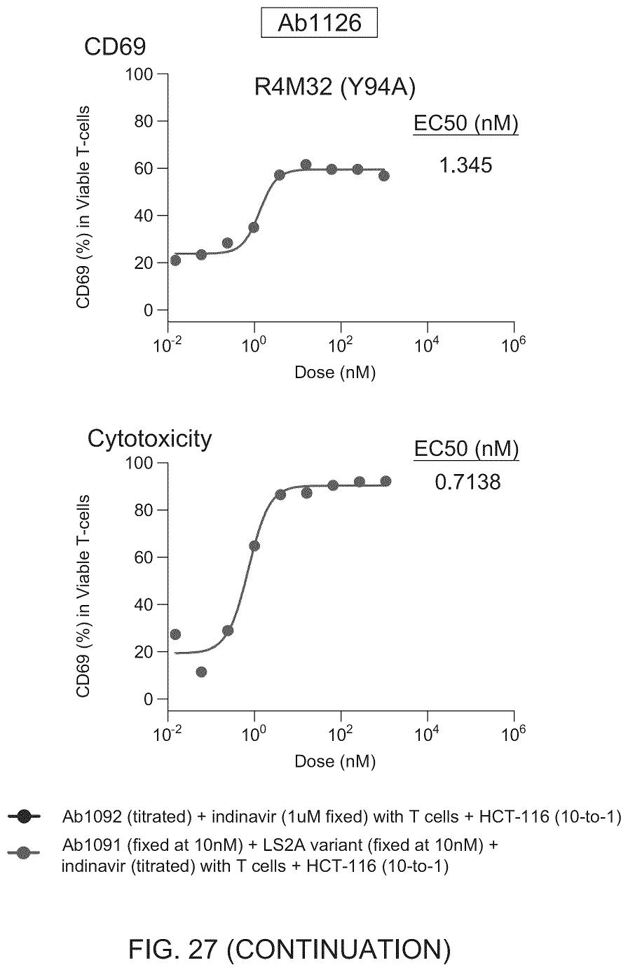

### Isolation of T Cells for In Vitro Assays (FIG. 24, FIG. 25, FIG. 26, FIG. 27, FIG. 30, FIG. 33, FIG. 35, FIG. 37, FIG. 38, FIG. 39, FIG. 40, FIG. 41, FIG. 42, FIG. 48)

Leukapheresis packs were obtained from deidentified healthy adult donors (StemCell Technologies, Vancouver, BC). Peripheral blood mononuclear cells (PBMCs) were first isolated using the EasySep Direct Human PBMC Kit (StemCell Technologies). Human T cells were magnetically isolated from the PBMCs using the EasySep Human T cell Isolation Kit (StemCell Technologies). T cells were cryopreserved in DMEM with 10% FBS and 10% DMSO and thawed the day of experiments.

### In Vitro Measurement of T-Cell Activation and TDCC (FIG. 24, FIG. 25, FIG. 26, FIG. 27, FIG. 30, FIG. 33, FIG. 35, FIG. 37, FIG. 38, FIG. 39, FIG. 40, FIG. 41, FIG. 48)

Co-culture assays were used to assess T-cell activation (CD69 upregulation), cytokine production or T-cell-dependent cellular cytotoxicity (TDCC) of target cells. Flat-bottom 96-well plates were seeded with 5,000 target cells in complete growth media (DMEM+10% FBS+1% penicillin/streptomycin) 16-24 hrs before the assay. Antibody drugs were added in 10 μL growth media and incubated for at least 10 min at 37° C. Indinavir or vehicle control was added in 10 μL growth media. Magnetically isolated human T-cells were thawed, washed, and resuspended in complete growth media, then added to target cells (50,000 T cells per well) in 80 μL growth media for a final assay well volume of 100 μL. The final effector:target (E:T) ratio was 10:1. Assay plates were incubated at 37° C. with 5% CO₂ for 66-70 hr depending on the experiment. T-cells were transferred to a U-bottom 384-well plate and spun (600 rcf×5 min). Supernatants were stored at −20° C. for later analysis of cytokines. T-cells were resuspended in CF405M viability dye (0.5 μM, Biotium, Fremont, CA) in PBS for 10 min at 37° C. At the end of viability staining, cells were fixed by adding methanol-free paraformaldehyde (Electron Microscopy Sciences, Hatfield, PA) to a final concentration of 1% for 10 min at room temperature. T-cells were spun (600 rcf×5 min) and supernatant was aspirated. T-cells were stained with FITC-conjugated anti-human CD45 (BioLegend, San Diego, CA) at 1 pg/mL final dilution and PE-conjugated anti-human CD69 (BioLegend, San Diego, CA) at 400 ng/mL final dilution for 30 minutes at room temperature in the dark, then immediately run an IntelliCyt® iQue3 flow cytometer (Sartorius AG, Göttingen, Germany). While T cells were being processed, target cells were incubated with CellTiter-Glo 2.0 (Promega, Madison, WI) for 15 min at room temperature with shaking then read for luciferase light emission on a GloMax Explorer plate reader using default settings. Specific Cytotoxicity (%) was calculated from the relative luminescence unit values (RLU) using the formula: [1−(experimental RLU/vehicle-treated RLU))×100]. The frequency of CD69+ T-cells (%) was calculated as the percentage of CD69-positive events within the CF405M-negative population. Dose-response curves and EC₅₀ values were calculated in GraphPad Prism 9 software (GraphPad Software San Diego, CA).

### Cytokine Measurement (FIG. 40 and FIG. 41)

At the conclusion of TDCC co-culture assays, the T-cells were separated from the target cells, centrifuged, and the growth media was transferred to a storage plate to be frozen at −20° C. until later analysis. Cytokine production was measured using the LEGENDplex™ Human Th1 Panel (5-plex) (BioLegend, San Diego, CA) according to the manufacturer's instructions and data acquisition on the iQue3 cytometer.

### Surface Staining of Human and Cyno PBMCs (FIG. 32):

Peripheral blood mononuclear cells (PBMCs) from healthy cynomolgus macaques (Chinese origin) were obtained commercially (iQ Biosciences, Berkeley, CA). Peripheral blood mononuclear cells (PBMCs) from healthy human donors were isolated from leukapheresis packs (leukopaks) from deidentified healthy adult donors (StemCell Technologies, Vancouver, BC). Human PBMCs were isolated using the EasySep Direct Human PBMC Kit (StemCell Technologies) then cryopreserved in DMEM with 10% FBS and 10% DMSO. Frozen PBMC cryovials were thawed on the day of experiments and washed complete growth media (DMEM+10% FBS+1% penicillin/streptomycin). Cells were resuspended in growth media and transferred to a 96-well U-bottom plate at 55,000 cell/well. Human TruStain FcX (Fc Receptor Blocking Solution; Catalog number 422302; BioLegend, San Diego, CA) was added at a 1:20 final dilution of the manufacturer's stock and incubated for 10 minutes at room temperature in a volume of 25 μL. Experimental antibodies diluted in CSM were added to the cells and incubated for 30 minutes at room temperature, in a total volume of 100 μL. Triplicate wells were prepared for each staining concentration. Plates were washed by adding 160 μL CSM, spinning 600 rcf×5 minutes and aspirating supernatant. The wash was repeated. Fluorescent conjugated antibodies (Brilliant Violet 650 anti-CD8 clone RPA-T8; PE conjugated anti-human IgG clone M1310G05; PE-Cγ7 anti-CD4 clone L200; Alexa 647 anti-TCRalpha/beta clone R73 or clone IP26) were obtained commercially from BioLegend (San Diego, CA) or BD Bioscience (San Jose, CA) and added at the manufacturer's recommended concentrations in a final volume of 100 μL then stained for 30 minutes at room temperature. Two washes were performed as above. Cells were resuspended in 60 μL DAPI Staining Solution (Catalog number GTX16206; GeneTex, Hsinchu City 300, Taiwan, China) at a final concentration of 1:10 of the manufacturer's stock solution. Cells were run immediately on an IntelliCyt® iQue3 flow cytometer (Sartorius AG, Göttingen, Germany).

### Surface Staining of Tumor Cell Lines (FIG. 34):

A single-cell suspension of adherent tumor cell lines was obtained by incubating 5 minutes with TrypLE Express (Gibco, Waltham, MA) and quenching with growth media (DMEM+10% FBS+1% penicillin/streptomycin), centrifuging at 400 rcf×5 minutes, aspirating the supernatant, and resuspending the cell pellet in CSM (AutoMACS Running Buffer, Miltenyi Biotec, Bergisch Gladbach, Germany).

Single-cell suspensions were transferred to a 96-well U-bottom plate at 30,000 cell/well and incubated with experimental antibodies diluted in CSM for 30 minutes at room temperature, in a total volume of 40 μL. Triplicate wells were prepared for each staining concentration. Plates were washed by adding 160 μL CSM, spinning 600 rcf×5 minutes and aspirating supernatant. The wash was repeated. Secondary antibody (PE conjugated anti-human Fc, catalog number 410708, BioLegend, San Diego, CA) was added at a final concentration of 5 pg/mL in a final volume of 30 μL and stained for 30 minutes at room temperature. Two washes were performed as above. Cells were resuspended in 60 μL DAPI Staining Solution (Catalog number GTX16206; GeneTex, Hsinchu City 300, Taiwan, China) at a final concentration of 1:10 of the manufacturer's stock solution. Cells were run immediately on an IntelliCyt® iQue3 flow cytometer (Sartorius AG, Göttingen, Germany).

### Real-Time Measurement of TDCC by Time-Lapse Microscopy (FIG. 42):

Co-cultures of magnetically isolated human T-cells and target cells were prepared as described above for TDCC assays, with some modifications. The target cells were HCT-116-NR cells, which are HCT-116 cells that were stably transduced with a nuclear-localized NucLight Red fluorophore (Sartorius AG, Göttingen, Germany). Co-cultures with appropriately diluted drugs were set up in flat-bottom 96-well plates in 150 μL final volume. Plates were imaged on an IncuCyte® S3 live-cell analysis system (Sartorius AG, Göttingen, Germany) once per hour starting when indinavir was added. Some wells were treated with a “quencher”, an IgG containing an anti-indinavir clone that blocks binding by LS2A but does not bind to LS2B. The IncuCyte software was used to quantify Normalized target cell abundance (red object area per image, normalized to T=0 h).

### Measurement of Ab1060 Pharmacokinetics in Cynomolgus Macaques (FIG. 45)

Two male and two female cynomolgus macaques (Mauritius origin) were distributed to 4 groups (n=1 per group). Animals were fasted overnight prior to dosing. Antibodies were dosed in alert animals at Time=0 hr via intravenous infusion using an infusion pump over 15 minutes. A postdose flush of saline was administered at a volume of approximately 1 mL. Blood (400 μL) was sampled serially at 13 time points post-infusion (1 hr, 3 hr, 6 hr, 1 d, 2 d, 3 d, 4 d, 5 d, 6 d, 7 d, 8 d, 9 d, and 10 d) with potassium EDTA as the anticoagulant, then processed to plasma and frozen. Plasma concentrations were quantified using an anti-human IgG sandwich ELISA that was developed and qualified in-house as described above. Pharmacokinetic parameters were calculated using the Phoenix WinNonLin software package (Certara, Princeton, NJ).

### Measurement of Ab1060 Cell Surface Binding in Cynomolgus Macaques (FIG. 46)

Two male and two female cynomolgus macaques (Mauritius origin) were distributed to 4 groups (n=1 per group). Animals were fasted overnight prior to dosing. Antibodies were dosed in alert animals at Time=0 hr via intravenous infusion using an infusion pump over 15 minutes. A postdose flush of saline was administered at a volume of approximately 1 mL. Blood (200 μL) was sampled serially at 8 time points post-infusion (1, 24, 48, 72, 96, 168, 192 and 240 hr) with potassium EDTA as the anticoagulant, then washed by centrifugation, and blocked with TruStain FcX (BioLegend, San Diego, CA) prior to antibody staining. Cells were stained for 30 minutes at room temperature with a panel of fluorescent conjugated antibodies (Brilliant Violet 650 anti-CD8 [BioLegend clone RPA-T8]; Brilliant Violet 605 anti-CD4 [BD Biosciences clone L200], AlexaFluor 700 anti-CD3 [BD Biosciences clone SP34-2], PE anti-human IgG Fc [BioLegend clone M1310G05]) in Brilliant Stain Buffer Plus (BD Biosciences, San Jose, CA). After staining, erythrocytes were lysed by adding ACK Lysis Solution, repeating lysis, and washing. Cells were run on a FACSAria Fusion Flow Cytometer (BD Biosciences). Data was analyzed with the FlowJo software package (BD Bioscience). CD4+ T cells were identified by gating on CD3+CD4+CD8-events. Median fluorescence intensity (MFI) of the PE channel in each sample was calculated and expressed as a normalized value relative to the sample with the highest MFI.

Measurement of Cytokines in Plasma from Transgenic B-hCD3e Mice (FIG. 47)

Transgenic B-hCDE mice on the C57BL/6 strain background were obtained from Biocytogen (Wakefield, MA). These mice were homozygous for a knock-in mutation in which exons 2-6 of the mouse Cd3e gene were replaced by exons 2-7 of the human CD3E gene. Mice (n=4 per group, female, age 7-10 weeks) were injected intravenously in the lateral tail vein with the indicated antibodies and doses. Serial blood draws from the tail vein were processed to EDTA plasma, diluted 10-fold with a cryoprotectant buffer (50% glycerol in PBS), then frozen at −80° C. until later analysis. Cytokine levels were measured using the MILLIPLEX® Mouse High Sensitivity T Cell Panel multiplex bead-based assay (MilliporeSigma) using Luminex technology.

### Generation of EpCAM-Expressing Cell Lines (FIG. 33, FIG. 34, FIG. 35 and FIG. 48)

For some experiments, immortalized cell lines were genetically engineered to express EpCAM. Lentiviral vectors were cloned with the coding region of human EpCAM or cynomolgus macaque EpCAM driven by a mammalian promoter (UBC, SV40, EF1-alpha, MinTK, TATA) and an antibiotic selection marker driven by a separate mammalian promoter. Cells were transduced, selected with the appropriate antibiotic, then expanded under antibiotic selection prior to characterizing surface expression by flow cytometry. Flow cytometry staining was performed with the anti-human EpCAM clone 9C4 conjugated to either PE or FITC fluorophores (BD Biosciences, San Jose, CA). For some cell lines, FACS was used to isolate single-cell clones which were subsequently expanded and characterized by surface staining.

What is claimed is:

1. (canceled)

2. An EpCAM binding antibody or fragment thereof that comprises:
(a) a variable heavy chain (vh) that comprises:
(i) a vh complementary determining region 1 (vhCDR1) having an amino acid sequence that comprises: SEQ ID NO: 2545, SEQ ID NO: 2745, SEQ ID NO: 2801, SEQ ID NO: 2809, SEQ ID NO: 2817, SEQ ID NO: 2825, SEQ ID NO: 2833, SEQ ID NO: 2841, SEQ ID NO: 2865, or any of these amino acid sequences having 1 to 3 mutations and that is capable of binding EpCAM;
(ii) a vh complementary determining region 2 (vhCDR2) having an amino acid sequence that comprises: SEQ ID NO: 2746, SEQ ID NO: 2810, SEQ ID NO: 2554, SEQ ID NO: 2546, SEQ ID NO: 2898, SEQ ID NO: 2890, or any of these amino acid sequences having 1 to 3 mutations and that is capable of binding EpCAM;
(iii) a vh complementary determining region 3 (vhCDR3) having an amino acid sequence that comprises: SEQ ID NO: 2547, SEQ ID NO: 2747, SEQ ID NO: 2851, SEQ ID NO: 2859, SEQ ID NO: 2907, or any of these amino acid sequences having 1 to 3 mutations and that is capable of binding EpCAM; and
(b) a variable light chain (vl) that comprises:
(i) a vl complementary determining region 1 (vlCDR1) having an amino acid sequence that comprises SEQ ID NO: 2548, SEQ ID NO: 2748 or either of these amino acid sequences having 1 to 3 mutations and that is capable of binding EpCAM;
(ii) a vl complementary determining region 2 (vlCDR2) having an amino acid sequence that comprises SEQ ID NO: 2549, SEQ ID NO: 2749 or either of these amino acid sequences having 1 to 3 mutations and that is capable of binding EpCAM; and
(iii) a vl complementary determining region 3 (vlCDR3) having an amino acid sequence that comprises: SEQ ID NO: 2550, SEQ ID NO: 2750, SEQ ID NO: 2918, SEQ ID NO: 2926, SEQ ID NO: 2934, SEQ ID NO: 2942, or any of these amino acid sequences having 1 to 3 mutations and that is capable of binding EpCAM.

3. The EpCAM binding antibody or fragment thereof of claim 2, wherein:
(a) the vhCDR1 has an amino acid sequence that comprises SEQ ID NO: 2745 or SEQ ID NO: 2745 having 1 to 3 mutations and that is capable of binding EpCAM;
(b) the vhCDR2 has an amino acid sequence that comprises SEQ ID NO: 2746 or SEQ ID NO: 2746 having 1 to 3 mutations and that is capable of binding EpCAM;
(c) the vhCDR3 has an amino acid sequence that comprises SEQ ID NO: 2747 or SEQ ID NO: 2747 having 1 to 3 mutations and that is capable of binding EpCAM;
(d) the vlCDR1 has an amino acid sequence that comprises SEQ ID NO: 2748 or SEQ ID NO: 2748 having 1 to 3 mutations and that is capable of binding EpCAM,
(e) the vlCDR2 has an amino acid sequence that comprises SEQ ID NO: 2749 or SEQ ID NO: 2749 having 1 to 3 mutations and that is capable of binding EpCAM, and
(f) the vlCDR3 has an amino acid sequence that comprises SEQ ID NO: 2750 or SEQ ID NO: 2750 having 1 to 3 mutations and that is capable of binding EpCAM.

4. The EpCAM binding antibody or fragment thereof of claim 2, wherein the antibody or the fragment thereof is humanized.

5. The EpCAM binding antibody or fragment thereof of claim 2, wherein the fragment of the EpCam binding antibody is a scFv.

6. The EpCAM binding antibody or fragment thereof of claim 2, wherein the antibody or fragment thereof further comprises a tumor-associated antigen binding domain, wherein the tumor-associated antigen binding domain binds to HER2 or CD20.

7. The EpCAM binding antibody or fragment thereof of claim 6, wherein the antibody or fragment thereof comprises an Fc domain that comprises one or more knob variants or one or more hole variants.

8. The EpCAM binding antibody or fragment thereof of claim 2, wherein the antibody or fragment thereof exhibits antibody dependent cell-mediated cytotoxicity.

9. An EpCAM binding domain or fragment thereof that comprises:
(a) a variable heavy chain (vh) that comprises:
(i) a vh complementary determining region 1 (vhCDR1) having an amino acid sequence that comprises SEQ ID NO: 2761 or SEQ ID NO: 2761 having 1 to 3 mutations and that is capable of binding EpCAM;
(ii) a vh complementary determining region 2 (vhCDR2) having an amino acid sequence that comprises SEQ ID NO: 2762 and that is capable of binding EpCAM;
(iii) a vh complementary determining region 3 (vhCDR3) having an amino acid sequence that comprises SEQ ID NO: 2763 or SEQ ID NO: 2763 having 1 to 3 mutations and that is capable of binding EpCAM;
(b) a variable light chain (vl) that comprises:
(i) a vl complementary determining region 1 (vlCDR1) having an amino acid sequence that comprises SEQ ID NO: 2764 or SEQ ID NO: 2764 having 1 to 3 mutations and that is capable of binding EpCAM,
(ii) a vl complementary determining region 2 (vlCDR2) having an amino acid sequence that comprises SEQ ID NO: 2765 or SEQ ID NO: 2765 having 1 to 3 mutations and that is capable of binding EpCAM, and
(iii) a vl complementary determining region 3 (vlCDR3) having an amino acid sequence that comprises SEQ ID NO: 2766 or SEQ ID NO: 2766 having 1 to 3 mutations and that is capable of binding EpCAM.

10. A method of treating cancer in a subject, the method comprising administering to the subject the EpCAM binding domain or fragment thereof of claim 9.

11. The method of claim 10, wherein the EpCAM binding domain or fragment thereof is humanized.

12. The method of claim 10, wherein the fragment of the EpCam binding domain is a scFv.

13. The method of claim 10, wherein the EpCAM binding domain or fragment thereof further comprises a tumor-associated antigen binding domain.

14. The method of claim 13, wherein the tumor-associated antigen binding domain binds to HER2 or CD20.

15. The method of claim 10, wherein the EpCAM binding domain or fragment thereof exhibits antibody dependent cell-mediated cytotoxicity.

16. The method of claim 10, wherein the administering is oral, intravenous, subcutaneous, or intratumorally.

17. A method of treating cancer in a subject, the method comprising administering to the subject an EpCAM binding antibody or fragment thereof that comprises:
(a) a variable heavy chain (vh) that comprises:
(i) a vh complementary determining region 1 (vhCDR1) having an amino acid sequence that comprises: SEQ ID NO: 2545, SEQ ID NO: 2745, SEQ ID NO: 2801, SEQ ID NO: 2809, SEQ ID NO: 2817, SEQ ID NO: 2825, SEQ ID NO: 2833, SEQ ID NO: 2841, SEQ ID NO: 2865, or any of these amino acid sequences having 1 to 3 mutations and that is capable of binding EpCAM;
(ii) a vh complementary determining region 2 (vhCDR2) having an amino acid sequence that comprises: SEQ ID NO: 2746, SEQ ID NO: 2810, SEQ ID NO: 2554, SEQ ID NO: 2546, SEQ ID NO: 2898, SEQ ID NO: 2890, or any of these amino acid sequences having 1 to 3 mutations and that is capable of binding EpCAM;
(iii) a vh complementary determining region 3 (vhCDR3) having an amino acid sequence that comprises: SEQ ID NO: 2547, SEQ ID NO: 2747, SEQ ID NO: 2851, SEQ ID NO: 2859, SEQ ID NO: 2907, or any of these amino acid sequences having 1 to 3 mutations and that is capable of binding EpCAM; and
(b) a variable light chain (vl) that comprises:
(i) a vl complementary determining region 1 (vlCDR1) having an amino acid sequence that comprises SEQ ID NO: 2548, SEQ ID NO: 2748 or either of these amino acid sequences having 1 to 3 mutations and that is capable of binding EpCAM;
(ii) a vl complementary determining region 2 (vlCDR2) having an amino acid sequence that comprises SEQ ID NO: 2549, SEQ ID NO: 2749 or either of these amino acid sequences having 1 to 3 mutations and that is capable of binding EpCAM; and
(iii) a vl complementary determining region 3 (vlCDR3) having an amino acid sequence that comprises: SEQ ID NO: 2550, SEQ ID NO: 2750, SEQ ID NO: 2918, SEQ ID NO: 2926, SEQ ID NO: 2934, SEQ ID NO: 2942, or any of these amino acid sequences having 1 to 3 mutations and that is capable of binding EpCAM.

18. The method of claim 17, wherein the EpCAM binding antibody or the fragment thereof is humanized.

19. The method of claim 17, wherein the fragment of the EpCam binding antibody is a scFv.

20. The method of claim 17, wherein the EpCAM binding antibody or fragment thereof further comprises a tumor-associated antigen binding domain.

21. The method of claim 20, wherein the tumor-associated antigen binding domain binds to HER2 or CD20.

22. The method of claim 17, wherein the EpCAM binding antibody or fragment thereof exhibits antibody dependent cell-mediated cytotoxicity.

23. The method of claim 17, wherein:
(a) the vhCDR1 has an amino acid sequence that comprises SEQ ID NO: 2745 or SEQ ID NO: 2745 having 1 to 3 mutations and that is capable of binding EpCAM;
(b) the vhCDR2 has an amino acid sequence that comprises SEQ ID NO: 2746 or SEQ ID NO: 2746 having 1 to 3 mutations and that is capable of binding EpCAM;
(c) the vhCDR3 has an amino acid sequence that comprises SEQ ID NO: 2747 or SEQ ID NO: 2747 having 1 to 3 mutations and that is capable of binding EpCAM;
(d) the vlCDR1 has an amino acid sequence that comprises SEQ ID NO: 2748 or SEQ ID NO: 2748 having 1 to 3 mutations and that is capable of binding EpCAM,
(e) the vlCDR2 has an amino acid sequence that comprises SEQ ID NO: 2749 or SEQ ID NO: 2749 having 1 to 3 mutations and that is capable of binding EpCAM, and
(f) the vlCDR3 has an amino acid sequence that comprises SEQ ID NO: 2750 or SEQ ID NO: 2750 having 1 to 3 mutations and that is capable of binding EpCAM.

24. The method of claim 17, wherein the administering is oral, intravenous, subcutaneous, or intratumorally.
